(19) United States
(12) Patent Application Publication
(10) Pub. No.: US 2026/0325630 A1
(43) Pub. Date: Oct. 22, 2026

(54) COMPOSITIONS AND METHODS FOR TREATING METASTATIC DISEASES

(71) Applicants: ImmunEdge, Inc., Redwood City, CA (US); The Board of Trustees of the Leland Stanford Junior University, Stanford, CA (US)

(72) Inventors: Bo YU, Mountain View, CA (US); Edgar G. ENGLEMAN, Atherton, CA (US); David Kung-Chun CHIU, Mountain View, CA (US); Kazukuni HAYASHI, Mountain View, CA (US); Xiangyue ZHANG, Sunnyvale, CA (US); Karin JOOSS, San Diego, CA (US); Shelley Erin ACKERMAN, Redwood City, CA (US); Alessandro PALUMBO, San Francisco, CA (US)

(21) Appl. No.: 19/578,576

(22) Filed: Mar. 25, 2026

Related U.S. Application Data
(63) Continuation of application No. PCT/US2026/020637, filed on Mar. 24, 2026.
(60) Provisional application No. 63/776,795, filed on Mar. 24, 2025.

(51) Int. Cl. A61K 39/395; A61K 9/127; A61K 31/7105; A61K 38/17; A61K 39/00; A61P 35/04; C07K 16/28

(52) U.S. Cl. CPC A61K 39/3955; A61K 9/127; A61K 31/7105; A61K 38/179; A61P 35/04; C07K 16/2863; A61K 2039/505; C07K 2317/565

(57) ABSTRACT

The present disclosure provides anti-EPOR antibodies, anti-EPO antibodies, and fragments thereof that specifically bind to an EPOR complex with high affinity, engineered EPOs, and engineered EPOR fusion proteins for treating metastatic diseases. The anti-EPOR antibodies, anti-EPO antibodies, engineered EPOs, and/or engineered EPOR fusion proteins can be used to treat patients.

### CROSS-REFERENCE

This application is a continuation of International Application No. PCT/US2026/020637, filed on Mar. 24, 2026, which claims the benefit of U.S. Provisional Application No. 63/776,795 filed on Mar. 24, 2025, each of which is incorporated by reference herein in its entirety.

### STATEMENT AS TO FEDERALLY SPONSORED RESEARCH

This invention was made with Government support under contract CA274511 awarded by the National Institutes of Health. The Government has certain rights in the invention.

### SEQUENCE LISTING

The instant application contains a Sequence Listing which has been submitted in electronically in XML file format and is hereby incorporated by reference in its entirety. Said XML copy, created on May 1, 2026, is named 62379-707_301_SL.xml and is 5,526,702 bytes in size.

### BACKGROUND OF THE DISCLOSURE

Erythropoietin (EPO) induces hematopoiesis by dimerizing EPO receptor (EPOR) molecules, which leads to the activation of the EPO receptor-associated Janus tyrosine kinase 2 (Jak2) and secondary signaling molecules such as signal transducer and activator of transcription 5 (Stat5; Brines and Cerami, Nat Rev Neurosci, 2005; 6:484-94). EPO acts by binding to EPOR which is expressed on erythroid progenitor cells to inhibit apoptosis and promote cell survival, proliferation, and differentiation in production of mature red blood cells. However, EPOR expression is not restricted to erythroid tissue. EPOR is also expressed in a number of non-hematopoietic tissues and elicits tissue protective effects in ischemic injury and promotes wound healing, cardiovascular protection, angiogenesis, neuroprotection, regulation of metabolic homeostasis, and bone remodeling.

Metastasis is the leading cause of cancer-related deaths globally, with the liver being one of the most frequent and detrimental sites of metastatic spread. Moreover, multiple studies have highlighted the pivotal role of the liver in immune tolerance-mediated disease progression, in both primary hepatocellular carcinoma (HCC) and as a metastatic site for other solid tumors.

### SUMMARY OF THE DISCLOSURE

The present disclosure provides compositions and methods for preventing cancer metastasis by preventing the expression, formation, activation, and circulation of an erythropoietin (EPO) protein-hetero-EPO receptor complex in a subject.

In an aspect, the present disclosure provides a method for treating or inhibiting cancer metastasis in a subject, comprising administering a pharmaceutically effective amount of a composition that i) inhibits or reduces binding of an erythropoietin (EPO) protein to an EPO receptor (EPOR) complex, wherein said EPO receptor complex comprises (a) at least two EPOR subunits or (b) at least one EPOR subunit and at least one CD131 subunit; ii) inhibits or reduces formation of an EPO receptor (EPOR) complex, wherein said EPO receptor complex comprises (a) at least two EPOR subunits or (b) at least one EPOR subunit and at least one CD131 subunit; iii) inhibits or reduces activation of an EPO receptor (EPOR) complex on a plurality of macrophages, wherein said EPO receptor complex comprises (a) at least two EPOR subunits or (b) at least one EPOR subunit and at least one CD131 subunit; iv) reduces an amount of circulating EPO protein in said subject; and/or v) reduces an expression level of an EPO receptor (EPOR) complex on a plurality of macrophages, wherein said EPO receptor complex comprises (a) at least two EPOR subunits or (b) at least one EPOR subunit and at least one CD131 subunit; thereby a) treating the cancer metastasis, b) inhibiting formation of a pre-metastatic niche in a distant site that is different from a location of a primary tumor, or c) inhibiting a metastatic nodule growth.

In some embodiments, said EPO receptor complex is a homo-EPO receptor complex that comprises at least two EPOR subunits. In some embodiments, said homo-EPO receptor complex comprises or consists of two EPOR subunits. In some embodiments, said EPO receptor complex is a hetero-EPO receptor complex that comprises at least one EPOR subunit and at least one CD131 subunit. In some embodiments, said hetero-EPO receptor complex comprises or consists of an EPOR subunit and a CD131 subunit. In some embodiments, said subject has an existing metastatic cancer. In some embodiments, said subject does not have an existing metastatic cancer. In some embodiments, the method further comprises inhibiting or reducing exacerbating said primary tumor.

In some embodiments, the method further comprises increasing CD8+ T cell/regulatory T cell ratio. In some embodiments, the method further comprises inhibiting development of regulator T cells (Tregs). In some embodiments, CD8+ T cells express Cluster of Differentiation 45 (CD45), CD3, CD8, Perforin, Interferon gamma (IFNγ), Granzyme B, or tumor necrosis factor alpha (TNFα). In some embodiments, Tregs express Cluster of Differentiation 4 (CD4), CD25, CD127, Forkhead Box P3 (FoxP3), CD39, protein tyrosine phosphatase receptor type C (CD45RA), Interleukin-2 (IL-2), or a Cytotoxic T-Lymphocyte Associated Protein 4 (CTLA-4).

In some embodiments, the method further comprises rendering said cancer metastasis sensitive to an immune checkpoint inhibitor. In some embodiments, said immune checkpoint inhibitor comprises a Cytotoxic T-Lymphocyte Associated Protein 4 (CTLA-4) inhibitor, a Programmed Death 1 (PD-1) inhibitor, or a Programmed Death Ligand 1 (PD-L1) inhibitor.

In some embodiments, said distant site is different from a location of said primary tumor or said distant site is a tissue that is distinct from a tissue where said primary tumor is located. In some embodiments, said distant site is located in liver. In some embodiments, said primary tumor is located in colon. In some embodiments, said primary tumor is located in breast. In some embodiments, said primary tumor is located in pancreas. In some embodiments, said primary tumor is located in ovary.

In some embodiments, said composition comprises an antibody or a functional fragment thereof. In some embodiments, said antibody or said functional fragment thereof selectively binds to an EPO receptor subunit, a CD131 subunit, or a combination thereof. In some embodiments, said composition comprises a compound or a salt thereof, wherein said compound is an inhibitor of hypoxia-inducible factor (HIF), IL-1α, IL-1β, TNF-α, IL-6, estrogen receptors, phospholipase C-γ1, or Cb1/p85/Episin-1 pathway. In some embodiments, said composition comprises a plurality of lipid nanoparticles (LNPs) comprising siRNA directed to hetero-EPO receptor on macrophages. In some embodiments, said composition comprises an engineered EPO receptor comprising an extracellular domain (ECD) of said EPO receptor subunit. In some embodiments, said composition further comprises a second antibody or a functional fragment thereof that selectively binds to an immune checkpoint protein. In some embodiments, said immune checkpoint protein comprises PD-1, PD-L1, or CTLA-4. In some embodiments, said immune checkpoint protein is PD-1.

In some embodiments, inhibiting or reducing said formation of said EPO protein-homo-EPO receptor complex, inhibiting or reducing said formation of said homo-EPO receptor between two EPO receptor subunits, inhibiting or reducing said activation of said homo-EPO receptor on said plurality of macrophages, reducing said circulating EPO protein amount, and/or reducing said expression level of homo-EPO receptor in said plurality of macrophages can inhibit formation of a pre-metastatic niche in a distant site that is different from a location of a primary tumor. In some embodiments, inhibiting or reducing said formation of said EPO protein-homo-EPO receptor complex, inhibiting or reducing said formation of said homo-EPO receptor between two EPO receptor subunits, inhibiting or reducing said activation of said homo-EPO receptor on said plurality of macrophages, reducing said circulating EPO protein amount, and/or reducing said expression level of homo-EPO receptor in said plurality of macrophages can inhibit a metastatic nodule growth.

In some aspects, provided herein is a method for treating or inhibiting cancer metastasis in a subject, comprising: administering a pharmaceutically effective amount of a composition that comprises (i) an antibody or a functional fragment thereof, or (ii) an engineered fusion protein or a functional fragment thereof, wherein said antibody or said functional fragment thereof selectively binds to a first target and a second target, wherein the first target comprises an erythropoietin (EPO) protein, an EPO receptor subunit, a CD131 subunit, or a combination thereof, wherein the second target comprises an immune checkpoint protein, and wherein said antibody or said functional fragment thereof comprises a first antigen-binding domain and a second antigen-binding domain.

In some embodiments, said binding of said antibody or said functional fragment thereof to said first target inhibits or reduces (i) binding of an erythropoietin (EPO) protein to an EPO receptor (EPOR) complex, wherein said EPO receptor complex comprises (a) at least two EPOR subunits or (b) at least one EPOR subunit and at least one CD131 subunit; (ii) formation of an EPO receptor (EPOR) complex, wherein said EPO receptor complex comprises (a) at least two EPOR subunits or (b) at least one EPOR subunit and at least one CD131 subunit; (iii) activation of an EPO receptor (EPOR) complex on a plurality of macrophages, wherein said EPO receptor complex comprises (a) at least two EPOR subunits or (b) at least one EPOR subunit and at least one CD131 subunit.

In some embodiments, said inhibiting or reducing (i) binding of an erythropoietin (EPO) protein to an EPO receptor (EPOR) complex, wherein said EPO receptor complex comprises (a) at least two EPOR subunits or (b) at least one EPOR subunit and at least one CD131 subunit; or (ii) formation of an EPO receptor (EPOR) complex, wherein said EPO receptor complex comprises (a) at least two EPOR subunits or (b) at least one EPOR subunit and at least one CD131 subunit inhibits immune tolerance. In some embodiments, said binding of said antibody or said functional fragment thereof to said second target inhibits immune checkpoint. In some embodiments, said inhibiting immune checkpoint inhibits immune tolerance.

In some embodiments, said first antigen-binding domain comprises: a heavy chain variable region (VH) comprising a VH complementarity determining region 1 (VH-CDR1) sequence, a VH-CDR2 sequence, and a VH-CDR3 sequence; and a light chain variable region (VL) comprising a VL-CDR1 sequence, a VL-CDR2 sequence, and a VL-CDR3 sequence; a VH and a kappa chain variable regions (VK); or a VH and a lambda chain variable regions. In some embodiments, said first antigen-binding domain and/or said second antigen-binding domain comprises a Fab, a Fab′, a (Fab′)2, a variable fragment (Fv), a single chain variable fragment (scFv), a scFv-Fc, a Fab-Fc, a VHH, a non-antibody scaffold, or a combination thereof. In some embodiments, said second antigen-binding domain comprises a VHH.

In some embodiments, said immune checkpoint protein comprises PD-1, PD-L1, or CTLA-4. In some embodiments, said immune checkpoint protein is PD-1.

In some embodiments, said antibody or said functional fragment thereof is an IgG, an IgM, an IgE, an IgA, an IgD, is derived therefrom, or a combination thereof. In some embodiments, said antibody or said functional fragment thereof comprises a monoclonal antibody, a grafted antibody, a chimeric antibody, a human antibody, a humanized antibody, or a combination thereof. In some embodiments, said first antigen-binding domain and/or said second antigen-binding domain is isolated, recombinant, synthetic, or a combination thereof. In some embodiments, said antibody or a functional variant thereof further comprise a peptide linker.

In some embodiments, said engineered fusion protein or said functional fragment thereof comprises an engineered EPO receptor comprising an extracellular domain (ECD) of said EPO receptor subunit. In some embodiments, said first target comprises an EPO receptor subunit, a CD131 subunit, or a combination thereof. In some embodiments, said subject has an existing metastatic cancer. In some embodiments, said subject does not have an existing metastatic cancer.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

A better understanding of features and advantages of the present disclosure will be obtained by reference to the following detailed description, which sets forth illustrative embodiments of the disclosure, and the accompanying drawings.

FIG. 1 shows the novel concept that immunoregulatory macrophages can be reprogrammed into co-stimulatory macrophages by restricting EPO-EPOR signaling, offering a potential strategy for treating and preventing liver metastasis. The EPO-EPOR signaling is investigated with a primary focus on macrophages in the contexts of (i) early events that enable liver metastasis, (ii) late events that lead to immune tolerance induction, and (iii) as a druggable target for treating and preventing metastatic diseases. Beyond the conceptual innovation of EPO-EPOR as a key immunoregulatory signaling axis in solid tumor liver metastases and tumor-specific immune tolerance induction, this disclosure introduces innovative model systems, technologies, concepts, and therapeutics.

FIGS. 2A-2F show the role of EPOR⁺ macrophages in modulating immune responses and therapeutic outcomes in HCC. FIG. 2A shows immune profiling of murine spontaneous HCC models with inflamed and noninflamed TME. FIG. 2B shows a comparison of plasma EPO levels between normal and HCC-bearing mice. FIG. 2C shows overall survival analysis of wild-type (WT) mice bearing non-inflamed HCC with or without EPO deficiency. FIG. 2D illustrates macrophages that represent the dominant EPOR⁺ cell population in both mouse and human HCC. FIG. 2E shows overall survival analysis for wild-type (WT) and EPOR^{ΔLysM} mice in non-inflamed HCC models. FIG. 2F illustrates two weeks post-tumor induction, WT and EPOR^{ΔLysM} mice were treated intraperitoneally with 2 mg/kg of αPD-1 or IgG serotype control every three days (5 doses total). Tumor growth was monitored using luciferase-based bioluminescence imaging, and both tumor growth and kinetics and overall survival were evaluated.

FIGS. 3A-3E show how ablation of EPOR in macrophages reduces liver metastatic seeding and liver metastasis (LM)-mediated immune tolerance. FIG. 3A shows a schematic representation of the intrasplenic injection model for studying liver metastasis. FIG. 3B shows liver metastasis and incidence rate in wild-type (WT) and EPOR^{ΔLysM} mice in the MC38 intrasplenic injection model. FIG. 3C shows schematic representation of the two-site injection model for studying liver metastasis. FIG. 3D shows a comparison of subcutaneous MC38 tumor growth between WT and EPOR^{ΔLysM} mice, with and without liver metastasis (LM). FIG. 3E shows the CD8+ T cell to CD4+ Treg ratio was assessed in WT and EPOR^{ΔLysM} mice with LM.

FIGS. 4A-4 F show a development and validation of a spontaneous liver metastasis model. FIG. 4A shows trichrome staining of primary PDAC tumors in mice. FIG. 4B shows a schematic representation of the generation of PLM lines, along with liver metastasis incidence rate for each line. FIG. 4C shows H&E histological analysis and bioluminescence imaging (IVIS) of liver metastases. FIG. 4D shows H&E histological analysis showing how a liver metastasis (LM) module forms adjacent to the portal vein. FIG. 4E shows flow cytometry analysis of dynamic changes in macrophage populations during different stages of liver metastasis (LM). FIG. 4F shows immunofluorescence staining of F4/80 (macrophages) in PLM1 liver metastases.

FIGS. 5A-5D show how ablation of EPOR in macrophages reduces liver metastasis incidence. FIG. 5A shows a schematic representation of the spontaneous liver metastasis model. FIGS. 5B-5C show the liver metastasis burden and incidence rate in wild-type (WT) and EPOR^{ΔLysM} mice with liver metastasis (LM). FIG. 5D shows immunohistochemistry for EPOR in human liver metastases.

FIG. 6 shows the increase in Tregs during the progression of liver metastases. Evaluation of the frequency of Tregs in the spontaneous PLM model at various timepoints.

FIGS. 7A-7D illustrates siEPOR-NPs selectively target macrophages and reduce the tumor burden of HCC-bearing mice. FIG. 7A shows the physical properties of the liposomes that were used for packaging siRNA. FIG. 7B illustrates flow cytometry analysis used to determine which myeloid cells in the liver took up the FITC-conjugated siNTC (non-target control)-NPs. FIG. 7C shows the knockdown efficiency of EPOR in liver macrophages determined by qPCR. FIG. 7D shows the effect of siEPOR-NPs on the tumor burden of non-inflamed HCC-bearing mice.

FIG. 8A illustrates generation of a spontaneous HCC model by in vivo delivery of plasmids pCMV-SB13, pT3-EF1a-Myc and pX330-sgRNA targeting Trp53, Pten, Keap1 to mouse liver via HDTV.

FIG. 8B illustrates overall survival C57BL/6 WT mice injected intraperitoneally (IP) with 2 mg/kg of αPD-1 (RMP1-14) or IgG Isotype control every 3 days, 2 weeks after HDTV (n=7-9/group).

FIG. 8C illustrates spleen weight and plasma EPO concentration of tumor-bearing mice 5 weeks after HDTV (n=8-10/group).

FIG. 8D illustrates data from blood samples collected from tumor-bearing mice with comparable tumor burdens and analyzed for complete blood count, 4 weeks after HDTV (n=5/group).

FIGS. 8E-8F illustrate correlation of EPO mRNA expression (EPO^{high}, upper quartile; EPO^{low}, lower quartile). FIG. 8E illustrates 5-year overall survival and FIG. 8F illustrates immune composition in HCC patients (TCGA and LIRI-JR).

FIG. 8G illustrates data from blood samples collected from tumor-bearing mice with comparable tumor burdens and measured for plasma EPO concentration (n=6/group).

FIG. 8H illustrates size measurements of tumors harvested 5 weeks after HDTV of Trp53^{KO}/Myc^{OE} and Trp53^{KO}/Epo^{KO}/Myc^{OE} HCC.

FIG. 8I illustrates intratumoral immune cell profiling of tumors harvested 5 weeks after HDTV for Trp53^{KO}/Myc^{OE} and Trp53^{KO}/Epo^{KO}/Myc^{OE} HCC mice (n=7-8/group).

FIG. 8J illustrates determination of tumor size and regression rate (CR: complete regression; PR: partial regression; NR: no regression) for Hepa1-6_EV (empty vector) and Hepa1-6_Epo^{OE} (EPO-overexpressing) tumors.

FIGS. 8K-8M illustrate intratumoral immune cell profiling for Hepa1-6_EV (empty vector) and Hepa1-6_Epo^{OE} (EPO-overexpressing) tumors. FIG. 8K illustrates intratumoral immune cell profiling for Hepa1-6_EV (empty vector) and Hepa1-6_EpoOE (EPO-overexpressing) tumors. FIG. 8L illustrates intratumoral immune cell profiling for Hepa1-6_EV (empty vector) and Hepa1-6_EpoOE (EPO-overexpressing) tumors. FIG. 8M illustrates intratumoral immune cell profiling for Hepa1-6_EV (empty vector) and Hepa1-6_EpoOE (EPO-overexpressing) tumors.

FIG. 8N shows tumor size and immune cell profiling of Hepa1-6_Epo^{OE} mice injected IP with αCD25, αCTLA-4, αCCR8 or IgG control.

FIG. 9A illustrates the major EPOR+ populations in Hepa1-6 tumors: F4/80⁺EPOR⁺ macrophages, which consisted of CD11b^{hi} MDMs, CD11b^{lo} MDMs and TIM-4⁺ KCs.

FIG. 9B illustrates EPOR and MHCII levels in Hepa1-6 tumors.

FIG. 9C illustrates the percentage of each F4/80⁺EPOR⁺ macrophage subset in Hepa1-6 tumors.

FIG. 9D illustrates EPOR and MHCII expression in Hepa1-6 tumors.

FIG. 9E illustrates the proportion of EGFP⁺ cells in liver KCs two weeks after orthotopic implantation of Hepa1-6_EV or Hepa1-6_Epo^{OE} in Ms4a3-EGFP reporter mice.

FIG. 9F illustrates the percentage of EPOR⁺CD68⁺ human macrophages and their CD14/CD163 levels in tumors (T) and adjacent non-tumor liver tissues (NT).

FIG. 9G illustrates the EPOR expression of EPOR⁺CD68⁺ human macrophages in T/NT pairs.

FIG. 9H illustrates representative immunofluorescent images showing overlapping staining pattern of EPOR and CD68 in human HCC.

FIG. 9I illustrates the correlation of EPO and EPOR mRNA expression in HCC patients (TCGA).

FIG. 9J illustrates the correlation of the signature enrichment (SE) scores of two liver macrophage subsets with EPOR mRNA expression, in human HCC-NT (TCGA).

FIG. 9K illustrates the top five upregulated pathways (from 72 genes) enriched in EPOR⁺ macrophages relative to EPOR macrophages (left), and the correlation between EPOR⁺ macrophage signature enrichment and cell type signature for five KC subsets (right).

FIG. 10A illustrates survival of HCC-bearing WT and Epor^{ΔLysM} mice (n=8/group).

FIG. 10B illustrates tumor growth kinetics measured by luciferin-based bioluminescence imaging (n=7/group) in HCC-bearing WT and Epor^{ΔLysM} mice.

FIG. 10C illustrates tumor growth kinetics 2 weeks after HDTV of Keap1^{KO}/Myc^{OE}-Luc⁺ mice injected (IP) with either PBS or 50 IU of rHuEPO daily for 3 weeks (n=8/group).

FIG. 10D illustrates overall survival in WT mice and Epor^{ΔLysM} mice after HDTV (Trp53^{KO}/Myc^{OE}-Luc⁺; Epo^{WT}) or (Trp53^{KO}/Epo^{KO}/Myc^{OE}-Luc⁺; Epo^{KO}) (n=8/group).

FIG. 10E illustrates tumor size and CR rate measurement for orthotopically implanted Hepa1-6_Epo^{OE} tumors from WT and Epor^{ΔLysM} mice harvested on day 21 (n=8-12/group).

FIG. 10F illustrates tumor burden in Hepa1-6_Epo^{OE} (n=6/group) HCC bearing mice after administration of liposomes containing 50 μg of siEpor or siNTC (non-targeted control).

FIG. 10G illustrates tumor burden in Trp53^{KO}/MycOE (n=8/group) HCC bearing mice after administration of liposomes containing 50 μg of siEpor or siNTC (non-targeted control).

FIG. 11A illustrates total leukocyte infiltration per gram of tumor, frequency of different immune populations and functional status of TAMs (CD11b⁺F4/80⁺CD64⁺) in Trp53^{KO}/Myc^{OE} tumors of WT and Epor^{ΔLysM} harvested 5 weeks after HDTV (n=6/group).

FIG. 11B illustrates overall survival of C57BL/6 WT or Epor^{ΔLysM} mice 2 weeks after HDTV (Trp53^{KO}/Myc^{OE}) injected (IP) with anti-CD8 mAb (YTS 169.4) or IgG control twice during the first week and then once weekly (total 6 doses).

FIG. 11C illustrates a UMAP plot displaying the distribution of CD8⁺ T cells in WT and Epor^{ΔLysM} groups (n=6/group; 2500 CD8⁺ T cells/sample).

FIG. 11D illustrates distribution percentage of each cluster along with the expression levels of 8 DEMs in WT and Epor^{ΔLysM} groups (n=6/group; 2500 CD8⁺ T cells/sample).

FIG. 11E illustrates tumor growth kinetics and overall survival in C57BL/6 WT and Epor^{ΔLysM} mice 2 weeks after HDTV (Trp53^{KO}/Myc^{OE}-Luc⁺), injected (IP) with 2 mg/kg of αPD-1 (RMP1-14) or IgG Isotype control every 3 days (total 5 doses). Gray area refers to the threshold level of background noise.

FIG. 11F illustrates overall survival (1.5 weeks after HDTV (Trp53^{KO}/Myc^{OE})) in C57BL/6 Epor^{ΔLYsM-ERT2} mice injected (IP) with 75 mg/kg tamoxifen or corn oil every 3 days (total 8 doses), in addition to the αPD-1 regimen.

FIG. 11G illustrates overall survival (2 weeks after HDTV (Trp53^{KO}/Myc^{OE})) in C57BL/6 WT mice injected (IP) with 20 mg/kg mEPOR-Fc or PBS weekly (total 4 doses), in addition to the αPD-1 regimen.

FIGS. 12A-12E illustrate flow cytometry and bulk-RNAseq data from KCs isolated by FACS from adjacent liver tissues of Hepa1-6_EV (KC_{Ev}) and Hepa1-6_Epo^{OE} (KC_{Epo}) tumors at day 18 post-implantation. FIG. 12A illustrates protein levels of functional markers in KC_{Ev} and KC_{Epo}. FIG. 12B illustrates principal component analysis of gene expression profiles of KC_{Ev} and KC_{Epo}. FIG. 12C illustrates Geneset enrichment analysis for inflammatory response gene signature and heatmap for core proinflammatory cytokines enriched in KC_{Ev}. FIG. 12D KC_{Ev} and KC_{Epo} volcano plots of differentially expressed genes in KC_{Ev} and KC_{Epo}. FIG. 12E illustrates the top five upregulated and downregulated pathways enriched in KC_{Epo} relative to KC_{Ev}.

FIG. 12F illustrates correlation between EPO and KC_{Epo} signatures in human HCC (TCGA).

FIGS. 12G-12I illustrates gene set enrichment analysis comparing the gene expression profiles of KC_{Ev} and KC_{Epo}. FIG. 12G illustrates that the KC_{Epo} profile was enriched for the human LILRB5⁺ immunoregulatory KC gene signature (GenesetILIRB5_KCs). FIG. 12H illustrates core genes enriched in KC_{Epo} for the GenesetILIRB5_KCs. FIG. 12I illustrates transcriptional factors that regulate the core genes (analyzed by ChEA3).

FIG. 13A illustrates the nucleus-to-cytoplasm ratio of NRF2 signal in KC_{EV} and KC_{Epo}.

FIGS. 13B-13E illustrates KCs and MDMs isolated from Hepa1-6_EV and Hepa1-6_Epo^{OE} HCC-bearing WT and Epor-deficient mice. FIGS. 13B-13C illustrates mRNA expression of iron metabolism-associated genes and antioxidant genes in KC_{EV} and KC_{Epo}. FIG. 13D illustrates mRNA expression of iron metabolism-associated genes and antioxidant genes in MDM_{EV} and MDM_{Epo}. FIG. 13E illustrates illustrates mRNA expression of iron metabolism-associated genes and antioxidant genes in KC_{Epo} with Epor deficiency.

FIG. 13F illustrates intracellular heme level in KCs isolated from Hepa1-6 tumors.

FIG. 13G illustrates intracellular heme level in TAMs (both KCs and MDMs) isolated from cold HCC tumors of WT and Epor^{ΔLysM} mice.

FIG. 13H illustrates tumor size and CR rate in orthotopically implanted Hepa1-6_Epo^{OE} tumors in WT and LysM^{Cre}, Nrf2^{fl/fl} (Nrf2^{ΔLysM}) mice harvested on Day 18 (n=6/group).

FIG. 13I illustrates representative H&E and CD8-stained images of partially regressed tumors in Nrf2^{ΔLysM} mice.

FIG. 13J illustrates survival of HCC bearing WT and Nrf2^{ΔLysM} mice after HDTV (Trp53^{KO}/Myc^{OE}) (n=8/group).

FIG. 13K illustrates the percentages of T cell populations in HCC tumors of WT and Nrf2^{ΔLysM} mice 5 weeks after HDTV (Trp53^{KO}/Myc^{OE}) (n=4/group).

FIGS. 14A-14B illustrates generation of spontaneous HCC models by in vivo delivery of plasmids pCMV=SB13, pT3=EF1a=Myc and pX330-sgRNA targeting Trp53, Pten, Keap1 to mouse liver via HDTV. FIG. 14A illustrates plasmid uptake measured by luciferin-based bioluminescence imaging in livers, lungs and spleens harvested 2 days after HDTV. FIG. 14B illustrates total leukocyte infiltration per gram of tumor, and frequency and functional status of different immune cell types analyzed by flow cytometry (n=8-10/group) from HCC tumors harvested 5 weeks after HDTV.

FIG. 15A illustrates complete blood count (CBC) of mice bearing inflamed or non-inflamed HCC 4 weeks after HDTV (n=5/group). FIG. 15B illustrates complete blood count (CBC) of mice bearing inflamed or non-inflamed HCC4 weeks after HDTV (n=5/group).

FIG. 16A illustrates correlations between EPO mRNA expression and vascular invasion and Edmondson-Steiner grade.

FIG. 16B illustrates correlations between EPO mRNA expression and various hypoxic markers in human HCC (TCGA), stratified by EPO expression quartiles (EPO^{high}: upper quartile, EPO^{low}: lower quartile).

FIG. 16C illustrates correlations between EPO mRNA expression and 5-year overall survival in human cancers (TCGA; ACC: adrenocortical carcinoma; KIRC: kidney renal clear cell carcinoma; SKCM: skin cutaneous melanoma; BRCA: breast invasive carcinoma; COADREAD: colorectal adenocarcinoma).

FIG. 17 illustrates establishment of a spontaneous non-inflamed HCC model with EPO-deficiency using a pX333 vector, as described herein.

FIG. 18A illustrates representative immunohistochemistry images of CD8⁺ T cells in Hepa1-6 tumors from wild-type C57BL/6 mice orthotopically implanted with allogeneic Hepa1-6 cells.

FIG. 18B illustrates size of Hepa1-6 tumors (day 21) in C57BL/6 wild-type (WT) and Rag2^{-/-} mice (n=9/group) orthotopically implanted with allogeneic Hepa1-6 cells.

FIG. 19A-1 illustrates characterization of Tregs in Hepa1-6-EV and Hepa1-6-Epo^{OE} tumors. FIG. 19A-2 illustrates characterization of Tregs in Hepa1-6-EV and Hepa1-6-Epo^{OE} tumors.

FIG. 19B illustrates the effects of EPO on in vitro Treg polarization in the presence or absence of TGFβ in Hepa1-6-EV and Hepa1-6-Epo^{OE} tumors.

FIG. 19C illustrates a comparison of CD25, CCR8, and CTLA-4 expression levels between Tregs and non-Tregs from Hepa1-6-EV and Hepa1-6-Epo^{OE} tumors.

FIG. 19D illustrates tumor size and regression rate measurements of Hepa1-6.Epo^{OE} on days 14, 17 and 20 after orthotopic implantation after IP injection with 2 mg/kg of αCD25, αCTLA-4, αCCR8 or IgG control. Tumors were harvested on day 21 (n=5/group).

FIG. 19E illustrates flow cytometry analysis of splenic Tregs.

FIG. 19F illustrates a representative Foxp3 immunohistochemistry image of αCTLA-4-treated tumors.

FIG. 19G illustrates a quantification of intratumoral Tregs and CD8⁺ T cells.

FIG. 20A illustrates the proportion of Ter119+CD71+ cells within the CD11b+Gr1+ population in tumors from various spontaneous HCC models.

FIG. 20B illustrates the proportion of Ter119+CD71+ cells within the CD11b+Gr1+ population in tumors from spontaneous non-inflamed HCC models with EPO deficiency.

FIG. 20C illustrates the proportion of Ter119+CD71+ cells within the CD11b+Gr1+ population in tumors from Hepa1-6 orthotopic implantation models.

FIG. 20D illustrates overall survival of mice 2 weeks after HDTV (Trp53^{KO}/Myc^{OE}) in C57BL/6 WT mice injected (IP) with 2 mg/kg of αPD-1 (RMP1-14), αTer-119 (TER-119) or IgG Isotype control every 3 days (total 9 doses).

FIG. 21A illustrates expression levels of EPOR and MHCII in macrophages from non-inflamed HCC, with and without EPO deficiency.

FIG. 21B illustrates F4/80⁺EPOR⁺ macrophages comprising CD11b^{hi} MDMs and CD11b^{lo} MDMs in HCC tumors of Epor-tdTomato 5 weeks after HDTV (Keap1^{KO}/Myc^{OE}).

FIG. 21C illustrates expression levels of CLEC4F and VSIG4 in different macrophage subsets from liver tissues of HCC-bearing mice (Hepa1-6 orthotopic implantation models).

FIG. 21D illustrates the proportion of EGFP⁺ cells in the KC population of liver tissues from Ms4a3-EGFP reporter mice 2 weeks after orthotopic implantation (Hepa1-6-EV or Hepa1-6-Epo^{OE}).

FIG. 22A illustrates immune profiling of EPOR⁺ cells in liver tissues from HCC patients, and FIG. 22B illustrates the corresponding gating strategy.

FIG. 22C illustrates representative immunofluorescence images of human HCC tissues stained for EPOR and CD68.

FIG. 23A illustrates correlation of core markers of two liver macrophage subsets with EPOR mRNA expression in human HCC-NT (TCGA). FIG. 23B illustrates correlation of core markers of two liver macrophage subsets with EPOR mRNA expression in human HCC-NT (TCGA).

FIG. 24 illustrates differential gene expression analysis in human tumor associated EPOR⁺ and EPOR macrophages.

FIG. 25A illustrates differentially expressed genes EPOR⁺ and EPOR⁻ macrophages in human HCC. FIG. 25B illustrates differentially expressed genes EPOR+ and EPOR− macrophages in human HCC. FIG. 25C illustrates differentially expressed genes EPOR+ and EPOR− macrophages in human HCC. FIG. 25D illustrates differentially expressed genes EPOR+ and EPOR− macrophages in human HCC.

FIG. 26 illustrates representative CD8 immunohistochemistry images of Hepa1-6_Epo^{OE} tumors in Epor^{fl/fl} or Epor^{ΔLysM} mice, harvested on Day 14.

FIG. 27 illustrates the proportion of Tregs in livers of Epor^{fl/fl} or Epor^{ΔlysM} mice injected (IP) with either PBS or 50 IU of rHuEPO daily for 7 days.

FIG. 28A illustrates the physical properties of the liposomes that were used for packaging siRNA, as described herein.

FIG. 28B illustrates immune cell uptake of liposomes (FITC⁺ population) in livers of wild-type mice injected with liposomes containing 50 μg of FITC-conjugated siNTC (non-target control) after 48 hours.

FIG. 28C illustrates the knockdown efficiency of Epor in macrophages in livers, as determined by qPCR, in wild-type mice I. V. injected with liposomes containing siNTC or siEpor.

FIG. 29 illustrates overall survival of C57BL/6 Epor^{ΔLysM} mice 2 weeks after HDTV (Trp53^{KO}/Myc^{OB}), injected (IP) with 4 mg/kg of αCD8 (YTS 169.4), αCD4 (GK1.5) or IgG control twice for the first week and then once weekly (total 6 doses) (n=8/group).

FIG. 30A illustrates a UMAP plot of CD8⁺ T cell populations from HCC-bearing wild-type and HCC-bearing Epor^{ΔLysM} mice.

FIG. 30B illustrates PhenoGraph and heatmap expression analysis of CD8⁺ T cell populations from HCC-bearing wild-type and HCC-bearing Epor^{ΔLysM} mice.

FIG. 31 illustrates UMAP plots and PhenoGraph analysis of the CD8⁺ T cell population identified clusters from HCC-bearing wild-type and HCC-bearing Epor^{ΔLysM} mice.

FIG. 32 illustrates UMAP plots of the CD8⁺ T cell populations from HCC-bearing wild-type and HCC-bearing Epor^{ΔLysM} mice (n=6/group; 2500 CD8⁺ T cells/sample). Heat mapping was used to visualize the relative expression of each marker in UMAP.

FIG. 33A illustrates differential gene expression analysis among KC_{EV} and KC_{Epo} cells isolated from adjacent liver tissues of Hepa1-6_EV (KC_{EV}) and Hepa1-6_Epo^{OE} (KC_{Epo}) on day 18 post-implantation.

FIG. 33B illustrates a heatmap for all differentially expressed genes (DEGs) in KC_{EV} and KC_{Epo} from adjacent liver tissues of Hepa1-6_EV (KC_{EV}) and Hepa1-6_Epo^{OE} (KC_{Epo}) on day 18 post-implantation.

FIG. 33C-1 and FIG. 33C-2 illustrates a heatmap for the top 50 differentially expressed genes (DEGs) in KC_{EV} and KC_{Epo} from adjacent liver tissues of Hepa1-6_EV (KC_{EV}) and Hepa1-6_Epo^{OE} (KC_{Epo}) on day 18 post-implantation.

FIG. 34A illustrates mRNA expression of DEGs identified by bulk-RNAseq in KCs isolated from Hepa1-6_EV and Hepa1-6_Epo^{OE} tumors in wild-type and Epor-deficient mice.

FIG. 34B illustrates mRNA expression of DEGs identified by bulk-RNAseq in MDMs isolated from Hepa1-6_EV and Hepa1-6_Epo^{OE} tumors in wild-type and Epor-deficient mice.

FIG. 35 illustrates the KC_{EV} profile is enriched for the human CDIC⁺ antigen-presenting KC gene signature.

FIG. 36A and FIG. 36B illustrates signaling pathway analysis of EPO-EPOR signaling in KCs isolated from adjacent liver tissues of Hepa1-6_EV (KC_{EV}) and Hepa1-6_Epo^{OE} (KC_{Epo}) tumors at day 18 post-implantation. Gene set enrichment analysis (Database: KEGG_MEICUS) was used to compare the gene expression profiles of KC_{EV} and KC_{Epo}. Twenty-nine out of 226 gene sets were upregulated in KC_{Epo}, and two gene sets were significantly enriched at nominal p-value <5%.

FIG. 37 illustrates heme depletion in human macrophages by EPO treatment.

FIG. 38A illustrates intracellular free iron levels in bone-marrow-derived macrophages in vitro after EPO treatment in wild-type and Epor^{ΔLysM} mice.

FIG. 38B illustrates intracellular free iron levels in macrophages isolated from cold HCC tumors of wild-type and Epor^{ΔLysM} mice.

FIGS. 39A-39D illustrate the amino acid sequence and nucleic acid sequence of human EPOR extracellular domain (ECD) or human CD131 ECD, human CD131 D3D4 domains, and human EPOR (F93A) domains, including the signal peptide sequences (underlined). FIG. 39A shows the amino acid sequence (SEQ ID NO: 65) and nucleic acid sequence (SEQ ID NO: 66) of human EPOR ECD in IME020 and IME061. FIG. 39B shows the amino acid sequence (SEQ ID NO: 67) and nucleic acid sequence (SEQ ID NO: 68) of human CD131 ECD in IME062. FIG. 39C shows the amino acid sequence (SEQ ID NO: 69) and nucleic acid sequence (SEQ ID NO: 70) of human CD131 D3D4 domain in IME063. FIG. 39D shows the amino acid sequence (SEQ ID NO: 71) and nucleic acid sequence (SEQ ID NO: 72) of human EPOR (F93A) domains in IME083 and IME034.

FIG. 40 shows that ablation of EPOR in macrophages leads to reduced tumor burden in mouse liver metastasis model. A schematic representation of the intrasplenic injection model for studying liver metastasis is shown (top). LysMCre; Eporfl/fl (Epor^{ΔLysM}) mice that develop EPOR-deficiency in the mature myeloid cells (primarily macrophages) were generated. Wild-type (WT) or EPOR^{ΔLysM} C57BL/6 mice were injected intrasplenically with 5×10⁵ MC38 cells (Conventional (intrasplenic) liver metastasis model), followed by splenectomy. Four weeks after intrasplenic injection, liver tissues were harvested to evaluate and compare tumor burden and the incidence of liver metastasis in WT and EPOR^{ΔLysM} mice (bottom).

FIG. 41 shows that ablation of EPOR in macrophages leads to reduced tumor burden in mouse pancreatic cancer model with liver metastasis. A schematic representation of the spontaneous liver metastasis model is shown (top). LysMCre; Eporfl/fl (Epor^{ΔLysM}) mice that develop EPOR-deficiency in the mature myeloid cells (primarily macrophages) were generated. Wild-type (WT) or EPOR^{ΔLysM} C57BL/6 mice were orthotopically injected in the pancreas with 1×10⁵ PLM1 cells (spontaneous liver metastasis model). Three weeks after injection, pancreatic and liver tissues were harvested to evaluate and compare primary pancreatic ductal adenocarcinoma (PDAC) and liver metastasis burden in WT and EPOR^{ΔLysM} mice (bottom).

FIG. 42 shows that ablation of EPOR in macrophages leads to reduced tumor burden in mouse colorectal cancer model with liver metastasis. A schematic representation of the spontaneous liver metastasis model is shown (top). LysMCre; Eporfl/fl (Epor^{ΔLysM}) mice that develop EPOR-deficiency in the mature myeloid cells (primarily macrophages) were generated. Wild-type (WT) or EPOR^{ΔLysM} C57BL/6 mice were orthotopically injected in the colon (or cecum) with 1.5×10⁵ CLM1 cells (spontaneous liver metastasis model). 28 days after injection, cecum and liver tissues were harvested to evaluate primary colorectal cancer (CRC) and liver metastasis burden in WT and Epor^{ΔLysM} mice (bottom).

FIG. 43 shows the treatment with anti-EPOR antibody leads to significant reduction in liver metastases in pancreatic metastasis mouse model. PLM1 cells were implanted into the pancreas of WT C57BL/6 male mice. Animals were treated with either vehicle or anti-EPOR mouse antibody weekly at dose 1, and liver metastatic burden was measured ex vivo 21 days post tumor implantation. n=9-10 animals per group (one animal in vehicle succumbed to tumor burden prior to endpoint). * indicates p value<0.05 by one-tailed unpaired T test. EpoR mAb 1: anti-EPOR mouse antibody 1.

FIG. 44 shows the treatment with anti-EPOR antibody leads to significant reduction in liver metastases in an ovarian metastasis tumor model. Tumor cells were implanted into the peritoneal cavity of female WT mice. Animals were dosed via intraperitoneal injection with vehicle or anti-EPOR mouse antibody weekly at dose 1. Total tumor burden was measured as total tumor weight. n=9-10 animals per group. * indicates p value<0.05 by one-tailed unpaired T test. EpoR mAb 2: anti-EPOR mouse antibody 2.

FIG. 45 shows the treatment with anti-EPOR antibody demonstrates significant long-term survival benefit in liver cancer mouse model. Hydrodynamic tail vein injection (HDTV) was performed using sterile saline and plasmid mixture with a total volume corresponding to 10% of total body weight of the mice over 8-10 seconds per animal. Animals were dosed with the indicated test article at dose 1 weekly for anti-EPOR antibody 1 or anti-EPOR antibody 2. The EPO trap was administered at dose 2, once a week and anti-PD-1 antibody was administered at dose 2, twice a week. Efficacy was measured by overall survival of animals. mAb 1 and mAb 2: anti-EPOR mouse antibody 1 and 2, respectively. KO: knock out. OE: overexpression. **=P<0.01. ***=P<0.0005.

### DETAILED DESCRIPTION OF THE DISCLOSURE

While various embodiments of the present disclosure are described herein, it will be obvious to those skilled in the art that such embodiments are provided by way of example only. Numerous modifications and changes to, and variations and substitutions of, the embodiments described herein will be apparent to those skilled in the art without departing from the disclosure. It is understood that various alternatives to the embodiments described herein can be employed in practicing the disclosure. It is also understood that every embodiment of the disclosure can optionally be combined with any one or more of the other embodiments described herein which are consistent with that embodiment.

Where elements are presented in list format (e.g., in a Markush group), it is understood that each possible subgroup of the elements is also disclosed, and any one or more elements can be removed from the list or group.

It is also understood that, unless clearly indicated to the contrary, in any method described or claimed herein that includes more than one act or step, the order of the acts or steps of the method is not necessarily limited to the order in which the acts or steps of the method are recited, but the disclosure encompasses embodiments in which the order is so limited.

It is further understood that, in general, where an embodiment in the description or the claims is referred to as comprising one or more features, the disclosure also encompasses embodiments that consist of, or consist essentially of, such feature(s).

It is also understood that any embodiment of the disclosure, e.g., any embodiment found within the prior art, can be explicitly excluded from the claims, regardless of whether or not the specific exclusion is recited in the specification.

It is further understood that reference to a peptide, a polypeptide or a protein herein, such as an antibody or a fragment thereof, includes pharmaceutically acceptable salts thereof unless specifically stated otherwise or the context clearly indicates otherwise. Such salts can have a positive net charge, a negative net charge or no net charge.

Headings are included herein for reference and to aid in locating certain sections. Headings are not intended to limit the scope of the embodiments and concepts described in the sections under those headings, and those embodiments and concepts may have applicability in other sections throughout the entire disclosure.

All patent literature and all non-patent literature cited herein are incorporated herein by reference in their entirety to the same extent as if each patent literature or non-patent literature were specifically and individually indicated to be incorporated herein by reference in its entirety.

Beyond erythroid progenitors, a growing body of evidence suggests broad EPOR expression in non-erythroid cells, such as hematopoietic stem cells (HSCs), megakaryocytes, B cells, T cells, macrophages (MΦs), endothelial cells, and neurons (Broxmeyer, J Exp Med 2013:210:205-208). Notably, the immune-modulatory role of EPO is increasingly recognized (Cantarelli et al., Am J Transplant 2019:19:2407-2414; Peng et al., Cell Death Dis 2020:11:79). The engagement of EPO signaling suppresses inflammatory responses by inhibiting the NFκB inducible immune pathway (Nairz et al., Immunity 2011:34:61-74). Moreover, EPO primes MPs for effective efferocytosis thereby preventing autoimmunity (Luo et al., Immunity 2016:44:287-302).

EPO is cardioprotective in ischemia reperfusion injury and myocardial infarction. EPO improves cardiac function linked to neovascularization mediated by stimulating coronary endothelial cells to activate endothelial nitric oxide (NO) synthase (eNOS) and NO production (Teng et al., Basic Res. Cardiol. 2011:106:343-354).

EPO stimulates neovascularization and angiogenesis by activating endothelial cells (ECs) and endothelial progenitor cells (EPCs) in physiological conditions and pathological conditions, e.g., ischemia cardio-vascular diseases and tumors. Activation of EPOR leads to mobilization, proliferation, migration, and differentiation of ECs and EPCs (Annese et al., Experimental Cell Research, 2019: 374(2):266-273).

In the central nervous system, EPO and EPOR are expressed by neurons, glial cells and cerebrovasculature endothelium. EPO was shown to be neurotrophic and neuroprotective in vitro and in animal models of neuronal injury associated with trauma, stroke, ischemia, inflammation and epileptic seizures. The beneficial effects of EPO were also demonstrated in clinical studies of stroke, schizophrenia and progressive multiple sclerosis. EPO protects neurons both directly, by preventing apoptosis, and indirectly, by modulating inflammatory processes and stimulating neurogenesis and angiogenesis (Wang et al., Stroke 2004:35:1732-7).

EPO regulation of metabolism extends beyond oxygen delivery and contributes to maintenance of white adipose tissue and metabolic homeostasis. EPO is protective in diet-induced obesity, improves glucose tolerance, reduces insulin resistance and regulates fat mass accumulation, particularly in male mice (Alnaeeli and Noguchi, Adipocyte 2015:4:153-157). EPO modulates the proinflammatory response of macrophage infiltration in white adipose tissue and promotes an anti-inflammatory phenotype by inhibiting expression of proinflammatory cytokines and reducing macrophage infiltration (Alnaeeli et al., Diabetes Metab. Res. Rev. 2014:63:2415-2431).

It has been shown that some of the cytoprotective effects of EPO are mediated through its binding to heterodimers containing a canonical EPOR and a common beta receptor (βcR or CD131; Brines et al., Proc Natl Acad Sci USA 2004; 101:14 907-14 912). Interestingly, carbamylated EPO binds to these heteroreceptors and exerts tissue-protective effects, whereas it does not bind to the classical EPOR (e.g., EPOR homodimers comprising two EPOR subunits) and does not stimulate erythropoiesis. βcR is not required for erythropoiesis. It is assumed that βcR in combination with the EPOR expressed by nonhematopoietic cells constitutes a tissue-protective receptor, thus creating a tissue-protective heteroreceptor.

The expression levels of EPO and EPOR are regulated. EPO production is induced under hypoxic conditions mediated by HIF (Semenza, Blood 2009:114(10):2015-9). Expression of EPOR is regulated by transcription factors Sp1, GATA1, and TAL1. Binding of EPO to EPOR on erythroid progenitor cells increases expression of transcription factors GATA1 and TAL1, that in turn transactivate EPOR expression (Suresh et al., Front Physiol. 2020:10:1534). EPOR is also regulated at the protein level. P85 promotes EPOR endocytosis and degradation. Prolyl hydroxylase D3 (PHD3) mediates proline hydroxylation of EPOR leading to proteasomal degradation. TFR2 and Scribble facilitate recycling of EPOR recycling (Bhoopalan et al., F1000Res. 2020; 9: F1000 Faculty Rev-1153).

It was recently found that EPOR plays a critical role in the induction of tumor immune tolerance by myeloid cells, including dendritic cells (DCs) and macrophages (MΦs in a wide range of primary and metastatic tumors, including liver metastasis-induced systemic antigen-specific immune tolerance. Moreover, EPOR is indispensable in myeloid cell-mediated tolerance in transplantation of allogeneic organs such as kidney, liver, lung, heart, etc.

### Definitions

Unless defined otherwise or clearly indicated otherwise by their use herein, all technical and scientific terms used herein have the same meaning as commonly understood by those of ordinary skill in the art to which this application belongs.

As used in the specification and the appended claims, the indefinite articles “a” and “an” and the definite article “the” can include plural referents as well as singular referents unless specifically stated otherwise or the context clearly indicates otherwise.

The term “about” or “approximately” means an acceptable error for a particular value as determined by one of ordinary skill in the art, which depends in part on how the value is measured or determined. In certain embodiments, the term “about” or “approximately” means within one standard deviation. In some embodiments, when no particular margin of error (e.g., a standard deviation to a mean value given in a chart or table of data) is recited, the term “about” or “approximately” means that range which would encompass the recited value and the range which would be included by rounding up or down to the recited value as well, taking into account significant figures. In certain embodiments, the term “about” or “approximately” means within ±10%, 5%, 4%, 3%, 2% or 1% of the specified value. Whenever the term “about” or “approximately” precedes the first numerical value in a series of two or more numerical values or in a series of two or more ranges of numerical values, the term “about” or “approximately” applies to each one of the numerical values in that series of numerical values or in that series of ranges of numerical values.

The term “antibody” can refer to a protein functionally defined as a binding protein and structurally defined as comprising an amino acid sequence that is recognized as being derived from the framework region of an immunoglobulin (Ig) encoding gene. An antibody can comprise one or more polypeptides substantially encoded by immunoglobulin genes or fragments of immunoglobulin genes. The recognized immunoglobulin genes can include the kappa, lambda, alpha, gamma, delta, epsilon and mu constant region genes, as well as myriad immunoglobulin variable region genes. Light chains can be classified as either kappa or lambda. Heavy chains can be classified as gamma, mu, alpha, delta, or epsilon, which in turn define the immunoglobulin classes, IgG, IgM, IgA, IgD and IgE, respectively. In some embodiments, these may be further divided into subclasses (isotypes), e.g., IgG1, IgG2, IgG3, IgG4, IgAQ1 and IgA2.

A typical gamma immunoglobulin (antibody) structural unit is known to comprise a tetramer. Each tetramer can be composed of two identical pairs of polypeptide chains, each pair having one “light” (about 25 kD) and one “heavy” chain (about 50-70 kD). The N-terminus of each chain can define a variable region of about 100 to 110 or more amino acids primarily responsible for antigen recognition. The terms variable light chain (V_{L}) and variable heavy chain (V_{H}) can refer to these light and heavy chains respectively.

Antibodies can exist as intact immunoglobulins or as a number of well-characterized fragments. Thus, for example, pepsin can digest an antibody below the disulfide linkages in the hinge region to produce F(ab)′₂, a dimer of Fab′ which itself is naturally a light chain joined to VH-CH1-Hinge by a disulfide bond. The F(ab)′₂ may be reduced under mild conditions to break the disulfide linkage/s in the hinge region thereby converting the (Fab′)₂ dimer into an Fab′ monomer. The Fab′ monomer is essentially a Fab with part of the hinge region (see, Fundamental Immunology, W. E. Paul, ed., Raven Press, N.Y. (1993), for a more detailed description of other antibody fragments). While various antibody fragments are defined in terms of the digestion of an intact antibody, one of skill in the art will appreciate that fragments can be synthesized de novo either chemically or by utilizing recombinant DNA methods. Thus, the term antibody, as used herein can also include antibody fragments either produced by the modification of whole antibodies or synthesized using recombinant DNA methodologies. Preferred antibodies can include V_{H}-V_{L} dimers, including single chain antibodies (antibodies that exist as a single polypeptide chain), such as single chain Fv antibodies (sFv or scFv) in which a variable heavy and a variable light region are joined together (directly or through a peptide linker) to form a continuous polypeptide. The single chain Fv antibody is a covalently linked V_{H}-V_{L} heterodimer which may be expressed from a nucleic acid including V_{H}- and V_{L}-encoding sequences either joined directly or joined by a peptide-encoding linker (e.g., Huston, et al. Proc. Nat. Acad. Sci. USA, 85:5879-5883, 1988, which is hereby incorporated by reference in its entirety). While the V_{H} and V_{L} are connected to each as a single polypeptide chain, the V_{H} and V_{L} domains associate non-covalently. Alternatively, the antibody can be another fragment. Other fragments can also be generated, including using recombinant techniques. For example Fab molecules can be displayed on phage if one of the chains (heavy or light) is fused to g3 capsid protein and the complementary chain exported to the periplasm as a soluble molecule. The two chains can be encoded on the same or on different replicons; the two antibody chains in each Fab molecule assemble post-translationally and the dimer is incorporated into the phage particle via linkage to one of the chains of g3p (see, e.g., U.S. Pat. No. 5,733,743, which is hereby incorporated by reference in its entirety). The scFv antibodies and a number of other structures converting the naturally aggregated, but chemically separated light and heavy polypeptide chains from an antibody V region into a molecule that folds into a three dimensional structure substantially similar to the structure of an antigen-binding site are known to those of skill in the art (see, e.g., U.S. Pat. Nos. 5,091,513, 5,132,405, and 4,956,778, all of which are hereby incorporated by reference in their entirety). Particularly preferred antibodies can include all those that have been displayed on phage or generated by recombinant technology using vectors where the chains are secreted as soluble proteins, e.g., scFv, Fv, Fab, (Fab′)₂. Antibodies can also include diabodies and minibodies.

Antibodies can also include heavy chain dimers, such as antibodies from camelids. Since the V_{H} region of a heavy chain dimer IgG in a camelid does not have to make hydrophobic interactions with a light chain, the region in the heavy chain that normally contacts a light chain is changed to hydrophilic amino acid residues in a camelid. V_{H} domains of heavy-chain dimer IgGs are called V_{HH} domains.

In camelids, the diversity of antibody repertoire can be determined by the complementary determining regions (CDR) 1, 2, and 3 in the V_{H} or V_{HH} regions. The CDR3 in the camel V_{HH} region can be characterized by its relatively long length averaging 16 amino acids (Muyldermans et al., 1994, Protein Engineering 7(9): 1129, which is hereby incorporated by reference in its entirety). This is in contrast to CDR3 regions of antibodies of many other species. For example, the CDR3 of mouse V_{H} can have an average of 9 amino acids.

Libraries of camelid-derived antibody variable regions, which maintain the in vivo diversity of the variable regions of a camelid, can be made by, for example, the methods disclosed in U.S. Patent Application publication No. US20050037421, published Feb. 17, 2005, which is hereby incorporated by reference in its entirety.

The terms “functional fragments,” “antigen-binding portions,” “antigen-binding fragments,” “antigen-binding domains,” or “antibody fragments” can be used interchangeably herein to refer to one or more fragments of an antibody that retain the ability to specifically bind to an antigen. Representative antigen-binding fragments can include, but are not limited to, a Fab, a Fab′, a (Fab′)₂, a Fv, a scFv, a dsFv, a variable heavy domain, a variable light domain, a variable NAR domain, bi-specific scFv, a bi-specific Fab₂, a tri-specific Fab₃, an AVIMER®, a minibody, a diabody, a maxibody, a camelid, a VHH, an intrabody, fusion proteins comprising an antibody portion (e.g., a domain antibody), a single chain binding polypeptide, a scFv-Fc, or a Fab-Fc.

In some instances, an antibody or functional fragment thereof can comprise an isolated antibody or functional fragment thereof, a purified antibody or functional fragment thereof, a recombinant antibody or functional fragment thereof, a modified antibody or functional fragment thereof, or a synthetic antibody or functional fragment thereof. It would be understood that the antibodies described herein can be modified as described herein or as known in the art. In some instances, antibodies and functional fragments thereof described herein can be partly or wholly synthetically produced. An antibody or functional fragment thereof can be a polypeptide or protein having a binding domain which can be or can be homologous to an antigen-binding domain. In some instances, an antibody or functional fragment thereof can be produced in an appropriate in vivo animal model and then isolated and/or purified.

The term “Fc region” can be used to define a C-terminal region of an immunoglobulin heavy chain. The “Fc region” can be a native sequence Fc region or a variant Fc region. The Fc region of an immunoglobulin generally can comprise two constant domains, CH2 and CH3.

In some instances, the Fc region of an immunoglobulin is important for many important antibody functions (e.g. effector functions), such as antigen-dependent cellular cytotoxicity (ADCC), complement dependent cytotoxicity (CDC), and antibody-dependent cell-mediated phagocytosis (ADCP), result in killing of target cells, albeit by different mechanisms. Accordingly, in some embodiments, the antibodies described herein comprise the variable domains of the invention combined with constant domains comprising different Fc regions, selected based on the biological activities of the antibody for the intended use. In certain instances, Human IgGs, for example, can be classified into four subclasses, IgG1, IgG2, IgG3, and IgG4, and each these of these comprises an Fc region having a unique profile for binding to one or more of Fcγ receptors (activating receptors FcγRI (CD64), FcγRIIA, FcγRIIC (CD32); FcγRIIIA and FcγRIIIB (CD16) and inhibiting receptor FcγRIIB), and for the first component of complement (C1q). Human IgG1 and IgG3 bind to all Fcγ receptors; IgG2 binds to FcγRIIA_{H131}, and with lower affinity to FcγRIIA_{R131} FcγRIIIA_{V158}; IgG4 binds to FcγRI, FcγRIIA, FcγRIIB, FcγRIIC, and FcγRIIIA_{V158}; and the inhibitory receptor FcγRIIB has a lower affinity for IgG1, IgG2 and IgG3 than all other Fcγ receptors. Studies have shown that FcγRI does not bind to IgG2, and FcγRIIIB does not bind to IgG2 or IgG4. Id. In general, with regard to ADCC activity, human IgG1≥IgG3≥IgG4≥IgG2.

In some embodiments, the antibodies of this disclosure are variants that possess reduced effector functions, which make it a desirable candidate for applications in which certain effector functions (such as complement fixation and ADCC) are unnecessary or deleterious. Such antibodies can have decreased complement-dependent cytotoxicity (CDC), antibody-dependent cell cytotoxicity (ADCC), or antibody dependent cellular phagocytosis (ADCP). In some embodiments, the antibodies of this disclosure are variants that possess increased effector functions for applications in which increased immunogenicity would be beneficial. Such antibodies can have increased CDC, ADCC, or ADCP, or a combination thereof. Non-limiting examples of in vitro assays to assess ADCC activity of a molecule of interest is described in U.S. Pat. Nos. 5,500,362 and 5,821,337. Alternatively, non-radioactive assays methods may be employed (e.g., ACTI™ and CytoTox 96® non-radioactive cytotoxicity assays). Useful effector cells for such assays include peripheral blood mononuclear cells (PBMC), monocytes, macrophages, and Natural Killer (NK) cells.

Antibodies can have increased half-lives and improved binding to the neonatal Fc receptor (FcRn) (See e.g., US 2005/0014934). Such antibodies can comprise an Fc region with one or more substitutions therein which improve binding of the Fc region to FcRn, and include those with substitutions at one or more of Fc region residues: 238, 256, 265, 272, 286, 303, 305, 307, 311, 312, 317, 340, 356, 360, 362, 376, 378, 380, 382, 413, 424 or 434 according to the EU numbering system (See e.g., U.S. Pat. No. 7,371,826). Other examples of Fc region variants are also contemplated (See e.g., Duncan & Winter, Nature 322:738-40 (1988); U.S. Pat. Nos. 5,648,260 and 5,624,821; and WO94/29351).

“Antibodies” can include, but are not limited to, monoclonal antibodies, polyclonal antibodies, chimeric antibodies, bispecific antibodies, multispecific antibodies, heteroconjugate antibodies, humanized antibodies, human antibodies, deimmunized antibodies, mutants thereof, fusions thereof, immunoconjugates thereof, antigen-binding fragments thereof, functional fragments thereof, and/or any other modified configuration of the immunoglobulin molecule that comprises an antigen recognition site of the required specificity, including glycosylation variants of antibodies, amino acid sequence variants of antibodies, and/or covalently modified antibodies.

An antibody can be a human antibody. A human antibody can be an antibody having an amino acid sequence corresponding to that of an antibody produced by a human and/or has been made using any of the techniques for making human antibodies known in the art or disclosed herein. This definition of a human antibody includes antibodies comprising at least one human heavy chain polypeptide or at least one human light chain polypeptide. One such example is an antibody comprising murine light chain and human heavy chain polypeptides. Human antibodies can be produced using various techniques known in the art. In one embodiment, the human antibody is selected from a phage library, where that phage library expresses human antibodies (Vaughan et al., 1996, Nature Biotechnology, 14:309-314; Sheets et al., 1998, PNAS USA, 95:6157-6162; Hoogenboom and Winter, 1991, J. Mol. Biol., 227:381; Marks et al., 1991, J. Mol. Biol., 222:581). Human antibodies can also be made by introducing human immunoglobulin loci into transgenic animals, e.g., mice in which the endogenous immunoglobulin genes have been partially or completely inactivated. This approach is described in U.S. Pat. Nos. 5,545,807; 5,545,806; 5,569,825; 5,625,126; 5,633,425; and 5,661,016. Alternatively, the human antibody may be prepared by immortalizing human B lymphocytes that produce an antibody directed against a target antigen (such B lymphocytes may be recovered from an subject or may have been immunized in vitro). See, e.g., Cole et al., Monoclonal Antibodies and Cancer Therapy, Alan R. Liss, p. 77 (1985); Boerner et al., 1991, J. Immunol., 147 (1):86-95; and U.S. Pat. No. 5,750,373.

As used herein, the term “binding specificity” of an antibody or “antibody specificity” can refer to the identity of the antigen to which the antibody binds, preferably to the identity of the epitope to which the antibody binds.

As used herein, the term “chimeric polynucleotide” can mean that the polynucleotide comprises regions which are wild-type and regions which are mutated. It may also mean that the polynucleotide comprises wild-type regions from one polynucleotide and wild-type regions from another related polynucleotide.

As used herein, the term “complementarity-determining region” or “CDR” can refer to the art-recognized term as exemplified by Kabat and Chothia. CDRs are also generally known as hypervariable regions or hypervariable loops (Chothia and Lesk (1987) J Mol. Biol. 196:901; Chothia et al. (1989) Nature 342:877; E. A. Kabat et al., Sequences of Proteins of Immunological Interest (National Institutes of Health, Bethesda, Md.) (1987); and Tramontano et al. (1990) J Mol. Biol. 215:175, all of which are hereby incorporated by reference in their entirety). “Framework region” or “FR” can refer to the region of the V domain that flank the CDRs. The positions of the CDRs and framework regions can be determined using various well known definitions in the art, e.g., Kabat, Chothia, international ImMunoGene Tics database (IMGT), and AbM (see, e.g., Johnson et al., supra; Chothia & Lesk, 1987, Canonical structures for the hypervariable regions of immunoglobulins. J. Mol. Biol. 196, 901-917; Chothia C. et al., 1989, Conformations of immunoglobulin hypervariable regions. Nature 342, 877-883; Chothia C. et al., 1992, structural repertoire of the human VH segments J. Mol. Biol. 227, 799-817; Al-Lazikani et al., J. Mol. Biol 1997, 273(4)). Definitions of antigen combining sites are also described in the following: Ruiz et al., IMGT, the international ImMunoGeneTics database. Nucleic Acids Res., 28, 219-221 (2000); and Lefranc, M.-P. IMGT, the international ImMunoGeneTics database. Nucleic Acids Res. January 1; 29(1):207-9 (2001); MacCallum et al, Antibody-antigen interactions: Contact analysis and binding site topography, J. Mol. Biol., 262 (5), 732-745 (1996); and Martin et al, Proc. Natl Acad. Sci. USA, 86, 9268-9272 (1989); Martin, et al, Methods Enzymol., 203, 121-153, (1991); Pedersen et al, Immunomethods, 1, 126, (1992); and Rees et al, In Sternberg M. J. E. (ed.), Protein Structure Prediction. Oxford University Press, Oxford, 141-172 1996, all of which are hereby incorporated by reference in their entirety).

As used herein, the term “affinity” can refer to the equilibrium constant for the reversible binding of two agents and is expressed as binding affinity (K_{D}). In some cases, K_{D} can be represented as a ratio of k_{off}, which can refer to the rate constant for dissociation of an antibody from the antibody or antigen-binding fragment/antigen complex, to kₒₙ, which can refer to the rate constant for association of an antibody, an antigen-binding domain, or an antigen-binding fragment to an antigen. Binding affinity may be determined using methods known in the art including, for example, surface plasmon resonance (SPR; Biacore™, real time molecular interaction monitoring system for analysis of affinity and/or kinetics), KinExA™ Biosensor (system for measuring binding affinity K_{D}), scintillation proximity assays, enzyme-linked immunosorbent assay (ELISA), ORIGEN immunoassay (IGEN), fluorescence quenching, fluorescence transfer, yeast display, or any combination thereof. Binding affinity may also be screened using a suitable bioassay. The binding affinity (K_{D}) of an antibody, antigen-binding domain, or antigen-binding fragment herein can be less than 600 nM, 590 nM, 580 nM, 570 nM, 560 nM, 550 nM, 540 nM, 530 nM, 520 nM, 510 nM, 500 nM, 490 nM, 480 nM, 470 nM, 460 nM, 450 nM, 440 nM, 430 nM, 420 nM, 410 nM, 400 nM, 390 nM, 380 nM, 370 nM, 360 nM, 350 nM, 340 nM, 330 nM, 320 nM, 310 nM, 300 nM, 290 nM, 280 nM, 270 nM, 260 nM, 250 nM, 240 nM, 230 nM, 220 nM, 210 nM, 200 nM, 190 nM, 180 nM, 170 nM, 160 nM, 150 nM, 140 nM, 130 nM, 120 nM, 110 nM, 100 nM, 90 nM, 80 nM, 70 nM, 50 nM, 50 nM, 49 nM, 48 nM, 47 nM, 46 nM, 45 nM, 44 nM, 43 nM, 42 nM, 41 nM, 40 nM, 39 nM, 38 nM, 37 nM, 36 nM, 35 nM, 34 nM, 33 nM, 32 nM, 31 nM, 30 nM, 29 nM, 28 nM, 27 nM, 26 nM, 25 nM, 24 nM, 23 nM, 22 nM, 21 nM, 20 nM, 19 nM, 18 nM, 17 nM, 16 nM, 15 nM, 14 nM, 13 nM, 12 nM, 11 nM, 10 nM, 9 nM, 8 nM, 7 nM, 6 nM, 5 nM, 4 nM, 3 nM, 2 nM, 1 nM, 990 pM, 980 pM, 970 pM, 960 pM, 950 pM, 940 pM, 930 pM, 920 pM, 910 pM, 900 pM, 890 pM, 880 pM, 870 pM, 860 pM, 850 pM, 840 pM, 830 pM, 820 pM, 810 pM, 800 pM, 790 pM, 780 pM, 770 pM, 760 pM, 750 pM, 740 pM, 730 pM, 720 pM, 710 pM, 700 pM, 690 pM, 680 pM, 670 pM, 660 pM, 650 pM, 640 pM, 630 pM, 620 pM, 610 pM, 600 pM, 590 pM, 580 pM, 570 pM, 560 pM, 550 pM, 540 pM, 530 pM, 520 pM, 510 pM, 500 pM, 490 pM, 480 pM, 470 pM, 460 pM, 450 pM, 440 pM, 430 pM, 420 pM, 410 pM, 400 pM, 390 pM, 380 pM, 370 pM, 360 pM, 350 pM, 340 pM, 330 pM, 320 pM, 310 pM, 300 pM, 290 pM, 280 pM, 270 pM, 260 pM, 250 pM, 240 pM, 230 pM, 220 pM, 210 pM, 200 pM, 190 pM, 180 pM, 170 pM, or any integer therebetween.

An antibody can selectively bind to a target if it can bind to a target with greater affinity, avidity, more readily, and/or with greater duration than it binds to other substances. For example, an anti-EPO antibody or functional fragment thereof that selectively binds to an EPO protein is an antibody or functional fragment that can bind this target with greater affinity, avidity, more readily, and/or with greater duration than it binds to a protein that is not an EPO protein.

As used herein, the term “EPO analog” can refer to a polypeptide having modifications of its polypeptide structure, or polypeptides having shorter, longer, and/or different amino acid sequence compared to wild-type human erythropoietin, and all of which bind with high affinity to the hetero-EPOR or the homo-EPOR. EPO analogs may be antagonists or agonists of the hetero-EPOR or homo-EPOR. EPO analogs may block the activity of the hetero-EPOR or the activity of the homo-EPOR. EPO analogs may activate the hetero-EPOR without activating the homo-EPOR. EPO analogs may activate the homo-EPOR without activating the hetero-EPOR. EPO analogs may inhibit the hetero-EPOR without inhibiting the homo-EPOR. EPO analogs may inhibit the homo-EPOR without inhibiting the hetero-EPOR. In some embodiments, EPO analogs can comprise anti-EPO antibodies, anti-homo-EPOR antibodies, or anti-hetero-EPOR antibodies. In some embodiments, EPO analogs can comprise antagonistic or agonistic anti-EPO antibodies, anti-homo-EPOR antibodies, or anti-hetero-EPOR antibodies.

Whenever the term “at least” or “greater than” precedes the first numerical value in a series of two or more numerical values, the term “at least” or “greater than” applies to each one of the numerical values in that series of numerical values.

The term “heterologous” can refer to an amino acid or nucleotide sequence that is not naturally found in association with the amino acid or nucleotide sequence with which it is associated.

As used herein, the term “immunotherapy” can refer to particular therapies aimed at modulating immune system components, such as antibodies or immunocytes, or by drugs or other agents that stimulate, inhibit or otherwise modulate the immune system. For example, “immunotherapy” can refer to checkpoint inhibitor therapy, adoptive cell therapy and/or autologous or allogeneic CAR T-cell therapy.

Whenever the term “no more than” or “less than” precedes the first numerical value in a series of two or more numerical values, the term “no more than” or “less than” applies to each one of the numerical values in that series of numerical values.

The term “polynucleotide” can refer to a polymer composed of nucleotide units. Polynucleotides can include naturally occurring nucleic acids, such as deoxyribonucleic acid (“DNA”) and ribonucleic acid (“RNA”), as well as nucleic acid analogs. Nucleic acid analogs can include those which contain non-naturally occurring bases, nucleotides that engage in linkages with other nucleotides other than the naturally occurring phosphodiester bond, or/and bases attached through linkages other than phosphodiester bonds. Non-limiting examples of nucleotide analogs can include phosphorothioates, phosphorodithioates, phosphorotriesters, phosphoramidates, boranophosphates, methylphosphonates, chiral-methyl phosphonates, 2-O-methyl ribonucleotides, peptide-nucleic acids (PNAs), and the like. Such polynucleotides can be synthesized, e.g., using an automated DNA synthesizer. The term “nucleic acid molecule” can refer to larger polynucleotides. The term “oligonucleotide” can refer to shorter polynucleotides. In certain embodiments, an oligonucleotide can comprise no more than about 50 nucleotides. It is understood that when a nucleotide sequence is represented by a DNA sequence (i.e., A, T, G, C), this also includes an RNA sequence (i.e., A, U, G, C) in which “U” replaces “T”.

The term “polypeptide” can refer to a polymer composed of natural or/and unnatural amino acid residues, naturally occurring structural variants thereof, or/and synthetic non-naturally occurring analogs thereof, linked via peptide bonds. Synthetic polypeptides can be synthesized, e.g., using an automated polypeptide synthesizer. Polypeptides can also be produced recombinantly in cells expressing nucleic acid sequences that encode the polypeptides. The term “protein” can refer to larger polypeptides. The term “peptide” can refer to shorter polypeptides. In certain embodiments, a peptide can comprise no more than about 50, about 40, or about 30 amino acid residues. Polypeptides can include antibodies and fragments thereof. Conventional notation is used herein to portray polypeptide sequences: the left-hand end of a polypeptide sequence is the amino (N)-terminus; the right-hand end of a polypeptide sequence is the carboxyl (C)-terminus.

Polypeptides can include one or more modifications that may be made during the course of synthetic or cellular production of the polypeptide, such as one or more post-translational modifications, whether or not the one or more modifications are deliberate. Modifications can include, without limitation, glycosylation (e.g., N-linked glycosylation and O-linked glycosylation), lipidation, phosphorylation, sulfation, acetylation (e.g., acetylation of the N-terminus), amidation (e.g., amidation of the C-terminus), hydroxylation, methylation, formation of an intramolecular or intermolecular disulfide bond, formation of a lactam between two side chains, formation of pyroglutamate, carbamylation, and ubiquitination. As another example, a polypeptide can be attached to a natural polymer (e.g., a polysaccharide) or a synthetic polymer (e.g., polyethylene glycol [PEG]), lipidated (e.g., acylated with a C₈-C₂₀ acyl group), or labeled with a detectable agent (e.g., a radionuclide, a fluorescent dye or an enzyme). PEGylation can increase the protease resistance, stability and half-life, increase the solubility and reduce the aggregation of the polypeptide.

The term “conservative substitution” can refer to substitution of an amino acid in a polypeptide with a functionally, structurally or chemically similar natural or unnatural amino acid. In certain embodiments, the following groups each contain natural amino acids that are conservative substitutions for one another:
1) Glycine (Gly/G), Alanine (Ala/A);
2) Isoleucine (Ile/I), Leucine (Leu/L), Methionine (Met/M), Valine (Val/V);
3) Phenylalanine (Phe/F), Tyrosine (Tyr/Y), Tryptophan (Trp/W);
4) Serine (Ser/S), Threonine (Thr/T), Cysteine (Cys/C);
5) Asparagine (Asn/N), Glutamine (Gln/Q);
6) Aspartic acid (Asp/D), Glutamic acid (Glu/E); and
7) Arginine (Arg/R), Lysine (Lys/K), Histidine (His/H).

In further embodiments, the following groups each contain natural amino acids that are conservative substitutions for one another:
1)non-polar: Ala, Val, Leu, Ile, Met, Pro (proline/P), Phe, Trp;
2) hydrophobic: Val, Leu, Ile, Phe, Tyr, Trp;
3) aliphatic: Ala, Val, Leu, Ile;
4) aromatic: Phe, Tyr, Trp, His;
5) uncharged polar or hydrophilic: Gly, Ala, Pro, Ser, Thr, Cys, Asn, Gln, Tyr (tyrosine may be regarded as a hydrophobic amino acid with a polar side group);
6) aliphatic hydroxyl- or sulfhydryl-containing: Ser, Thr, Cys;
7) amide-containing: Asn, Gln;
8) acidic: Asp, Glu;
9) basic: Lys, Arg, His; and
10) small: Gly, Ala, Ser, Cys.

In other embodiments, amino acids may be grouped as set out below:
1) hydrophobic: Val, Leu, Ile, Met, Phe, Trp, Tyr;
2) aromatic: Phe, Tyr, Trp, His;
3) neutral hydrophilic: Gly, Ala, Pro, Ser, Thr, Cys, Asn, Gln;
4) acidic: Asp, Glu;
5) basic: Lys, Arg, His; and
6) residues that influence backbone orientation: Pro, Gly.

A polypeptide having one or more modifications relative to a parent polypeptide may be called an “analog”, “derivative” or “variant” of the parent polypeptide as appropriate. In some embodiments, a variant can comprise a conservatively modified amino acid sequence variant or a conservatively substituted amino acid sequence. For example, a variant of a polypeptide or an antibody can comprise one or more conservative amino acid substitutions.

The disclosure encompasses pharmaceutically acceptable salts of polypeptides, including those with a positive net charge, those with a negative net charge, and those with no net charge.

The term “pharmaceutically acceptable” can refer to a substance (e.g., an active ingredient or an excipient) that is suitable for use in contact with the tissues and organs of a subject without excessive irritation, allergic response, immunogenicity and toxicity, is commensurate with a reasonable benefit/risk ratio, and is effective for its intended use. A “pharmaceutically acceptable” excipient or carrier of a pharmaceutical composition is also compatible with the other ingredients of the composition. The term “Pharmaceutically acceptable” can refer to a material, such as a carrier or diluent, which does not abrogate the biological activity or properties of the compound, and is relatively nontoxic, i.e., the material may be administered to an individual without causing undesirable biological effects or interacting in a deleterious manner with any of the components of the composition in which it is contained. A pharmaceutically acceptable excipient can denote any pharmaceutically acceptable ingredient in a pharmaceutical composition having no therapeutic activity and being non-toxic to the subject administered, such as disintegrators, binders, fillers, solvents, buffers, tonicity agents, stabilizers, antioxidants, surfactants, carriers, diluents, excipients, preservatives or lubricants used in formulating pharmaceutical products. Pharmaceutical compositions can facilitate administration of the compound to an organism and can be formulated in a conventional manner using one or more pharmaceutically acceptable inactive ingredients that facilitate processing of the active compounds into preparations that can be used pharmaceutically. A proper formulation is dependent upon the route of administration chosen and a summary of pharmaceutical compositions can be found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pennsylvania 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999), herein incorporated by reference. In some embodiments, pharmaceutical compositions can be formulated by dissolving active substances (e.g., anti-EPOR or anti-CD131 antibodies described herein) in aqueous solution for injection into disease tissues or disease cells. In some embodiments, pharmaceutical compositions can be formulated by dissolving active substances (e.g., anti-EPOR or anti-CD131 antibodies described herein) in aqueous solution for direct injection into disease tissues or disease cells.

The term “stringent hybridization conditions” can refer to hybridizing in 50% formamide at 5×SSC at a temperature of 42° C. and washing the filters in 0.2×SSC at 60° C. (1×SSC is 0.15 M NaCl, 0.015 M sodium citrate.) Stringent hybridization conditions also encompasses low ionic strength and high temperature for washing, for example 0.015 M sodium chloride/0.0015 M sodium citrate/0.1% sodium dodecyl sulfate at 50° C.; hybridization with a denaturing agent, such as formamide, for example, 50% (v/v) formamide with 0.1% bovine serum albumin/0.1% Ficoll/0.1% polyvinylpyrrolidone/50 mM sodium phosphate buffer at pH 6.5 with 750 mM sodium chloride, 75 mM sodium citrate at 42° C.; or 50% formamide, 5×SSC (0.75 M NaCl, 0.075 M sodium citrate), 50 mM sodium phosphate (pH 6.8), 0.1% sodium pyrophosphate, 5×Denhardt's solution, sonicated salmon sperm DNA (50 μg/ml), 0.1% SDS, and 10% dextran sulfate at 42° C., with washes at 42° C. in 0.2×SSC (sodium chloride/sodium citrate) and 50% formamide at 55° C., followed by a high-stringency wash consisting of 0.1×SSC containing EDTA at 55° C.

The term “subject” can refer to an animal, including, but not limited to, a mammal, such as a primate (e.g., a human, a chimpanzee or a monkey), a rodent (e.g., a rat, a mouse, a guinea pig, a gerbil or a hamster), a lagomorph (e.g., a rabbit), a swine (e.g., a pig), an equine (e.g., a horse), a canine (e.g., a dog) or a feline (e.g., a cat). Additional examples of mammals can include, but are not limited to, any member of the mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. In some cases, the mammal is a human. In some instances, the subject is an adult, a child, or an infant. In some cases, the subject may be an animal. In some cases, an animal may comprise human beings and non-human animals. In one embodiment, a non-human animal may be a non-human mammal described herein. In some instances, the subject is a companion animal. In some instances, the subject is a feline, a canine, or a rodent.

The term “substantially homologous” or “substantially identical” in the context of two polypeptides or polynucleotides can refer to two or more sequences or subsequences that have at least about 50%, 60%, 65%, 70%, 75%, 80%, 85%, 90%, 91%, 92%, 93%, 94%, 95%, 96%, 97%, 98%, or 99% amino acid or nucleic acid residue sequence identity, when compared and aligned for maximum correspondence, as measured using a sequence comparison algorithm or by visual inspection. The terms “substantially homologous” or “substantially identical” can mean at least about 70% amino acid or nucleic acid residue identity. The term “substantially homologous” or “substantially identical” can mean at least about 85% amino acid or nucleic acid residue sequence identity. The substantial homology or identity can exist over a region of the sequences that is at least about 20, 30, 40, 50, 100, 150, or 200 residues in length. The sequences can be substantially homologous or identical over the entire length of either or both comparison biopolymers.

Optimal alignment of sequences for comparison can be conducted, e.g., by the local homology algorithm of Smith and Waterman, Adv. Appl. Math., 2:482 (1981); by the homology alignment algorithm of Needleman and Wunsch, J. Mol. Biol., 48:443 (1970); by the search for similarity method of Pearson and Lipman, Proc. Natl. Acad. Sci. USA, 85:2444 (1988); by computerized implementations of these algorithms (e.g., GAP, BESTFIT, FASTA, and TFASTA in the Wisconsin Genetics Software Package, Genetics Computer Group, Madison, Wisconsin); or by visual inspection.

One example of a useful algorithm is PILEUP. PILEUP creates a multiple sequence alignment from a group of related sequences using progressive, pairwise alignments to show relationship and percent sequence identity. It also plots a tree or dendogram showing the clustering relationships used to create the alignment. PILEUP uses a simplification of the progressive alignment method of Feng and Doolittle, J. Mol. Evol., 35:351-360 (1987). The method used is similar to the method described by Higgins and Sharp, CABIOS, 5:151-153 (1989). The program can align up to about 300 sequences, each having a maximum length of about 5,000 nucleotides or amino acids. The multiple alignment procedure begins with the pairwise alignment of the two most similar sequences, producing a cluster of two aligned sequences. This cluster is then aligned to the next most related sequence or cluster of aligned sequences. Two clusters of sequences are aligned by a simple extension of the pairwise alignment of two individual sequences. The final alignment is achieved by a series of progressive, pairwise alignments. The program is run by designating specific sequences and their amino acid or nucleotide coordinates for regions of sequence comparison and by designating the program parameters. For example, a reference sequence can be compared to other test sequences to determine the percent sequence identity relationship using the following parameters: default gap weight (3.00), default gap length weight (0.10), and weighted end gaps. Another algorithm that is useful for generating multiple alignments of sequences is Clustal W (see, e.g., Thompson et al., Nucleic Acids Research, 22:4673-4680 [1994]).

Another example of an algorithm that is suitable for determining percent sequence identity and sequence similarity is the BLAST algorithm, which is described in Altschul et al., J. Mol. Biol., 215:403-410 (1990). Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul 1990). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction is halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults, e.g., a wordlength (W) of 11, an expectation (E) of 10, M=5, N=−4, and a comparison of both strands. For amino acid sequences, the BLASTP program uses as defaults, e.g., a wordlength (W) of 3, an expectation (E) of 10, and the BLOSUM62 scoring matrix (see Henikoff and Henikoff, Proc. Natl. Acad. Sci. USA, 89:10915 [1989]).

In addition to calculating percent sequence identity, the BLAST algorithm also performs a statistical analysis of the similarity between two sequences (see, e.g., Karlin and Altschul, Proc. Natl. Acad. Sci. USA, 90:5873-5787 [1993]). One measure of similarity provided by the BLAST algorithm is the smallest sum probability [P(N)], which provides an indication of the probability by which a match between two nucleotide or amino acid sequences would occur by chance. In certain embodiments, a polynucleotide is considered similar to a reference sequence if the smallest sum probability in a comparison of the test polynucleotide to the reference polynucleotide is less than about 0.1, 0.01 or 0.001.

A polypeptide can be substantially homologous or identical to a second polypeptide if the two polypeptides differ only by conservative amino acid substitutions. Two nucleic acid sequences can be substantially homologous or identical if the two polynucleotides hybridize to each other under stringent conditions, or under highly stringent conditions, as described herein.

The term “therapeutically effective amount” can refer to an amount of a composition or compound that, when administered to a subject, is sufficient to prevent, reduce the risk of developing, delay the onset of, slow the progression or cause regression of the medical condition being treated, or to alleviate to some extent the medical condition or one or more symptoms or complications of that condition. The term “therapeutically effective amount” can also refer to an amount of a composition or compound that is sufficient to elicit the biological or medical response of a cell, tissue, organ, system, animal or human which is sought by a researcher, veterinarian, medical doctor or clinician.

The terms “treat”, “treating” and “treatment” can include alleviating, ameliorating or abrogating a medical condition or one or more symptoms or complications associated with the condition, alleviating, ameliorating or eradicating one or more causes of the condition, preventing additional symptoms, inhibiting the disease or the condition, e.g., arresting the development of the disease or the condition, relieving the disease or the condition, causing regression of the disease or the condition, relieving a condition caused by the disease or the condition, or stopping the symptoms of the disease or the condition either prophylactically and/or therapeutically. In some embodiments, treating a disease or condition cam comprise reducing the size of disease tissues or disease cells. In some embodiments, treating a disease or a condition in a subject can comprise increasing the survival of a subject. In some embodiments, treating a disease or condition can comprise reducing or ameliorating the severity of a disease, delaying onset of a disease, inhibiting the progression of a disease, reducing hospitalization of or hospitalization length for a subject, improving the quality of life of a subject, reducing the number of symptoms associated with a disease, reducing or ameliorating the severity of a symptom associated with a disease, reducing the duration of a symptom associated with a disease, preventing the recurrence of a symptom associated with a disease, inhibiting the development or onset of a symptom of a disease, or inhibiting of the progression of a symptom associated with a disease. In some embodiments, treating a cancer can comprise reducing the size of tumor or increasing survival of a patient with a cancer. Reference to “treatment” of a medical condition can include prevention of the condition. The terms “prevent”, “preventing” and “prevention” can include precluding, reducing the risk of developing and delaying the onset of a medical condition or one or more symptoms or complications associated with the condition.

### Metastatic Disease

Metastatic disease is the leading cause of cancer-related mortality worldwide, with the liver being one of the most frequent sites of metastasis for solid tumors. Colorectal cancer (CRC) and pancreatic ductal adenocarcinoma (PDAC) exhibit particularly high incidences of liver metastasis, affecting over 50% and 75% of patients, respectively. The liver is important in immune tolerance-mediated disease progression, both in the context of primary hepatocellular carcinoma (HCC) and as a secondary site for metastasis from other solid tumors, but the underlying mechanisms driving liver metastasis remain poorly understood.

The liver is a unique organ that inherently promotes local and systemic immune tolerance to foreign antigens. In the context of liver metastasis, the immune response to tumor antigens presented within the liver leads to systemic suppression of antitumor immunity, enabling primary tumors to evade immunosurveillance and diminishing the efficacy of immune checkpoint blockade (ICB) therapies in both patients and preclinical models. Although the underlying mechanisms are not fully understood, recent studies in tumor-bearing mice with liver metastasis have shown that tumor-specific CD8+ effector T cells undergo apoptosis, while CD4+ regulatory T cells (Tregs) are activated, through interactions with liver macrophages. Lymph node metastasis is a critical event that can trigger the development of tumor-specific Tregs, further facilitating metastatic disease progression. While metastasis-induced immune tolerance is important in disease progression, the upstream mechanisms governing these pathobiological pathways remain insufficiently understood.

Erythropoietin (EPO), a glycoprotein hormone primarily recognized for its canonical role in stimulating red blood cell production, has recently been found to mediate other biological functions. For instance, EPO promotes efferocytosis in macrophages, a process of ingesting apoptotic cells and debris without triggering an immune response against self-antigens; this is vital for preventing excessive inflammation and autoimmune disease. The analysis of human hepatocellular carcinoma (HCC samples) from The Cancer Genome Atlas (TCGA) and International Cancer Genome Consortium (ICGC) databases revealed that elevated EPO expression in tumors is associated with worsened survival, macrovascular invasion, and poorer tumor differentiation by Edmondson-Steiner grading. In addition, EPO binds to its cognate receptor, EPOR, on myeloid cells to induce immune tolerance. As shown in the studies, in the liver this EPO-EPOR signaling axis drives macrophages to acquire an immune-modulatory phenotype, resulting in a non-inflamed (“cold”) tumor microenvironment (TME) that evades both immunosurveillance and ICB therapies (Chiu, et al. Tumor-derived erythropoietin acts as an immunosuppressive switch in cancer immunity. Science. 2025 Apr. 25; 388(6745):eadr3026. doi: 10.1126/science.adr3026. Epub 2025 Apr. 25. PMID: 40273234; PMCID: PMC12110762). Based on the above, EPO-EPOR signaling can be similarly co-opted to promote liver metastasis and Examples described herein can be used to (i) investigate EPOR+ macrophages as the primary mediators permitting liver metastasis, (ii) assess whether EPO-EPOR signaling in liver metastases drives tumor-specific immune tolerance induction, and (iii) examine the pharmacological inhibition of EPO-EPOR signaling as an effective strategy to prevent, inhibit, reduce or treat liver metastasis. In some embodiments, the EPO-EPOR signaling mechanism can enable metastasis to other organs and tissues in addition to the liver.

Macrophages are the dominant immune cell population in the liver, with the functional ability to induce both local and systemic immune tolerance to foreign antigens. Studies in hepatocellular carcinoma (HCC) have shown EPO secreted by tumor cells can bind to its cognate receptor (EPOR) on macrophages. This signaling pathway programs macrophages to adopt an immunoregulatory phenotype, contributing to the development of a non-inflamed (“cold”) tumor microenvironment (TME) that can evade immunosurveillance and resist immune checkpoint blockade (ICB) therapies. This EPO-EPOR signaling axis, initially characterized in HCC, can be similarly co-opted for liver metastasis and can promote tumor-specific immune tolerance. In some embodiments, EPOR⁺ macrophages in the liver can play a critical role in the development of liver metastasis. In some embodiments, therapeutic strategies aimed at disrupting the EPO/EPOR signaling pathway can prevent, inhibit, reduce, or treat liver or other metastasis.

In some embodiments, EPOR+ macrophages can be mediators that permit liver metastasis. Primary tumors remotely activate macrophages in the liver to prime a pre-metastatic niche, enhancing the liver's susceptibility to cancer cell colonization. Examples described herein can be used to investigate the role of EPOR+ macrophages in liver metastasis. In some embodiments, syngeneic liver-tropic PDAC lines, which spontaneously metastasize to the liver following orthotopic injection into the pancreas, can be used to study EPOR+ macrophages in cancer metastasis. In some embodiments, (a) the role of macrophages as gatekeepers in liver metastatic seeding; (b) EPO-EPOR activation in macrophages as a key driver of pre-metastatic niche development in the liver; (c) spatiotemporal changes in macrophage phenotype and cell-cell interactions during pre-metastatic niche formation and early stages of liver metastases; and/or (d) the generalizability of findings from murine tissues to human disease can be studied.

In some embodiments, metastatic nodules can locally reprogram liver macrophages to promote the expansion of regulatory T cells (Tregs), which then disseminate systemically to foster tumor-specific immune tolerance. In some embodiments, two experimental liver metastasis models can be used to examine: (a) EPO-EPOR signaling at the metastatic site as a driver for immune tolerance induction; (b) macrophages as mediators of metastasis-induced tumor-specific Treg development; and/or (c) liver metastases as an educational hub for Treg development.

In some embodiments, targeting the EPO/EPOR pathway (e.g., using any compositions described herein) can serve as both a preventive and therapeutic strategy for liver metastasis. In some embodiments, targeting the EPO/EPOR pathway (e.g., using any compositions described herein) can enhance the responsiveness of liver metastasis to ICB therapy. For example, monoclonal antibodies and decoy fusion proteins against EPOR (e.g., engineered EPO trap or EPOR fusion proteins), can be used to investigate: (a) the impact of EPO/EPOR-targeted therapies on liver metastasis incidence and tumor burden; and/or (b) whether ablating EPO/EPOR signaling in macrophages sensitizes tumor-bearing mice with liver metastasis to ICB. In some embodiments, administration of any treatments described herein at various time points—both before and/or after liver metastases—can prevent, inhibit, reduce, and/or treat liver or other metastatic diseases.

In some embodiments, by applying high-resolution analytical tools to syngeneic liver metastasis models, the critical mechanism(s) enabling tumor liver invasion and tolerance induction can be identified. In some embodiments, novel agents designed to prevent, inhibit, reduce, or even eliminate liver metastases can be used to treat metastatic diseases. In some embodiments, the mechanism responsible for liver metastasis and the agents that target them can be applied broadly to other sites of metastasis.

In some aspects, provided herein are methods for treating or inhibiting cancer metastasis in a subject in need thereof. In some embodiments, provided herein are methods for inhibiting formation of a pre-metastatic niche in a distant site that is different from a location of a primary tumor. In some embodiments, a distant site can comprise a site that is different from a location of said primary tumor. In some embodiments, a distant site can comprise a tissue that is distinct from a tissue where a primary tumor is located. In some embodiments, provided herein are methods for inhibiting a metastatic nodule growth. In some embodiments, the method can comprise administering a pharmaceutically effective amount of a composition that inhibits or reduces binding of an EPO protein to an EPOR complex. In some embodiments, the method can comprise administering a pharmaceutically effective amount of a composition that inhibits or reduces formation of an EPOR complex. In some embodiments, the method can comprise administering a pharmaceutically effective amount of a composition that inhibits or reduces activation of an EPOR complex on a plurality of macrophages. In some embodiments, the method can comprise administering a pharmaceutically effective amount of a composition that reduces an amount of circulating EPO protein in a subject. In some embodiments, the method can comprise administering a pharmaceutically effective amount of a composition that reduces an expression level of an EPOR complex on a plurality of macrophages.

In some embodiments, EPOR complex comprises at least two EPOR subunits (e.g., homodimer or homo-EPOR). In some embodiments, EPOR complex can comprise at least one EPOR subunit and at least one CD131 subunit (e.g., heterodimer or hetero-EPOR). In some embodiments, an EPOR complex can comprise a homo-EPO receptor complex comprising at least two EPOR subunits. In some embodiments, a homo-EPOR complex can comprise two EPOR subunits. In some embodiments, a homo-EPOR complex can consist of two EPOR subunits. In some embodiments, an EPOR complex can comprise a hetero-EPOR complex comprising at least one EPOR subunit and at least one CD131 subunit. In some embodiments, a hetero-EPOR complex can comprises an EPOR subunit and a CD131 subunit. In some embodiments, a hetero-EPOR complex can consist of an EPOR subunit and a CD131 subunit.

### Anti-EPOR, Anti-CD131, and Anti-EPO Antibodies

In some aspects, provided herein, are antibodies, antigen-binding fragments thereof, or functional fragments thereof that can selectively binds to a target. In some embodiments, antibodies, antigen-binding fragments thereof, or functional fragments thereof described herein can bind to an antigen of a target protein or an epitope on an antigen of a target protein.

In some embodiments, an antibody can be a monospecific antibody and binds a single epitope. For example, a monospecific antibody can have a plurality of immunoglobulin variable domain sequences, each of which binds the same epitope. In some embodiments, an antibody can be a bispecific antibody. A bispecific antibody can have specificity for no more than two antigens. A bispecific antibody can be characterized by a first immunoglobulin variable domain sequence which has binding specificity for a first epitope and a second immunoglobulin variable domain sequence that has binding specificity for a second epitope. In some embodiments, the first and second epitopes can be on the same antigen, e.g., the same protein (or subunit of a multimeric protein). In some embodiments, the first and second epitopes can overlap. In some embodiments, the first and second epitopes do not overlap. In some embodiments, the first and second epitopes can be on different antigens, e.g., different proteins (or different subunits of a multimeric protein). In some embodiments, a bispecific antibody can comprise a heavy chain variable domain sequence and a light chain variable domain sequence which have binding specificity for a first epitope and a heavy chain variable domain sequence and a light chain variable domain sequence which have binding specificity for a second epitope. In some embodiments, a bispecific antibody can comprise a half antibody having binding specificity for a first epitope and a half antibody having binding specificity for a second epitope. In some embodiments, a bispecific antibody can comprise a half antibody, or a fragment thereof, having binding specificity for a first epitope and a half antibody, or a fragment thereof, having binding specificity for a second epitope. In some embodiments, a bispecific antibody can comprise a scFv or a Fab, or fragment thereof, have binding specificity for a first epitope and a scFv or a Fab, or fragment thereof, have binding specificity for a second epitope.

In some embodiments, an antibody can be a multispecific or multifunctional antibody. For example, a multispecific or multifunctional antibody can comprise a plurality of immunoglobulin variable domains sequences, wherein a first immunoglobulin variable domain sequence of the plurality has binding specificity for a first epitope and a second immunoglobulin variable domain sequence of the plurality has binding specificity for a second epitope. In some embodiments, the first and second epitopes are on the same antigen, e.g., the same protein (or subunit of a multimeric protein). In some embodiments, the first and second epitopes can overlap. In some embodiments, the first and second epitopes may not overlap. In some embodiments, the first and second epitopes can be on different antigens, e.g., different proteins (or different subunits of a multimeric protein). In some embodiments a multispecific antibody can comprise a third, a fourth or a fifth immunoglobulin variable domain. In some embodiments, a multispecific antibody can be a bispecific antibody, a trispecific antibody, or a tetraspecific antibody. In some embodiments, multispecific antibodies can optionally further comprise one or more additional binding domain(s) that selectively bind(s) to an IgE, a FcεRIα, a FcεRII, a tumor associated antigen (FAA), or a combination thereof. Any bispecific or multispecific antibodies described herein can be isolated, purified, recombinant, synthetic, or any combination thereof. A bispecific or mutispecific antibodies described herein can be made via any suitable method and may be recombinant, synthetic, or a combination thereof. In one aspect, provided herein can be a liquid composition or a lyophilized composition comprising one or more of bispecific or multispecific antibodies described herein. In one embodiment, a composition can comprise a population of a bispecific or multispecific antibodies. In another embodiments, a composition can comprise a population of two, three, four, five, six, seven, eight, nine, ten, or more bispecific or multispecific antibodies described above. A bispecific or multispecific antibodies described herein can be utilized in an in vitro assay to, for example, identify and/or purify one or more tumor cell(s) from a mixed culture (e.g., a biological sample such as a biopsy or a blood sample). A bispecific or multispecific antibodies described herein can be utilized in an in vivo animal model to test the therapeutic efficacy of the bispecific or multispecific antibodies against a tumor.

In some embodiments, an antibody can comprise a diabody, and a single-chain molecule, as well as an antigen-binding fragment of an antibody (e.g., Fab, F(ab′)2, and Fv). For example, an antibody molecule can include a heavy (H) chain variable domain sequence (abbreviated herein as VH), and a light (L) chain variable domain sequence (abbreviated herein as VL). In some embodiments, an antibody can comprise a heavy chain and a light chain (referred to herein as a half antibody. In another example, an antibody can comprise two heavy (H) chain variable domain sequences and two light (L) chain variable domain sequence, thereby forming two antigen-binding sites, such as Fab, Fab′, F(ab′)2, Fc, Fd, Fd′, Fv, single chain antibodies (scFv for example), single variable domain antibodies, diabodies (Dab) (bivalent and bispecific), and chimeric (e.g., humanized) antibodies, which may be produced by the modification of whole antibodies or those synthesized de novo using recombinant DNA technologies. These functional antibody fragments can retain the ability to selectively bind with their respective antigen. Antibodies and antibody fragments can be from any class of antibodies including, but not limited to, IgG, IgA, IgM, IgD, and IgE, and from any subclass (e.g., IgG1, IgG2, IgG3, and IgG4) of antibodies. A preparation of antibodies can be monoclonal or polyclonal. An antibody can also be a human, humanized, CDR-grafted, or in vitro generated antibody. An antibody can have a heavy chain constant region chosen from, e.g., IgG1, IgG2, IgG3, or IgG4. An antibody can also have a light chain chosen from, e.g., kappa or lambda. The term “immunoglobulin” (Ig) is used interchangeably with the term “antibody” herein.

Non-limiting examples of antigen-binding fragments of an antibody can include: a Fab fragment (a monovalent fragment consisting of the VL, VH, CL and CH1 domains); a F(ab′)2 fragment (a bivalent fragment comprising two Fab fragments linked by a disulfide bridge at the hinge region); a Fd fragment consisting of the VH and CH1 domains; a Fv fragment consisting of the VL and VH domains of a single arm of an antibody; a diabody (dAb) fragment consisting of a VH domain; a camelid or camelized variable domain; a single chain Fv (scFv) (see, e.g., Bird et al. (1988) Science 242:423-426; and Huston et al. (1988) Proc. Natl. Acad. Sci. USA 85:5879-5883); and a single domain antibody. These antibody fragments can be obtained using conventional techniques known to those with skill in the art, and the fragments can be screened for utility in the same manner as are intact antibodies. For example, a single-chain antibody (scFV) can be engineered (see, for example, Colcher, D. et al. (1999) Ann N Y Acad Sci 880:263-80; and Reiter, Y. (1996) Clin Cancer Res 2:245-52). In some embodiments, a single chain antibody can be dimerized or multimerized to generate multivalent antibodies having specificities for different epitopes of the same target protein. In some embodiments, antibodies can include intact molecules as well as functional fragments thereof. Constant regions of antibodies can be altered or mutated to modify one or more properties of antibodies (e.g., to increase or decrease one or more of: Fc receptor binding, antibody glycosylation, the number of cysteine residues, effector cell function, or complement function). Methods for altering antibody constant regions are known in the art. In some embodiments, antibodies with altered function, e.g., altered affinity for an effector ligand, such as FcR on a cell, or the C1 component of complement can be produced by replacing at least one amino acid residue in the constant portion of the antibody with a different residue (see, e.g., EP 388,151 A1, U.S. Pat. Nos. 5,624,821 and 5,648,260, the contents of all of which are hereby incorporated by reference).

In some embodiments, antibodies, antigen-binding fragments thereof, or functional fragments thereof described herein can include a non-antibody scaffold. Non-limiting examples of non-antibody scaffolds include Affibodies, Affilins, Anticalins, Atrimers, Avimers, Bicyclic peptides, Cys-knots, DARPins, FN3 scaffolds (e.g., adnectins, centyrins, pronectins, Tn3), Fynomers, Kunitz domains, or OBodies.

In some embodiments, antibodies, antigen-binding fragments thereof, or functional fragments thereof described herein can be derivatized or linked to another functional molecule (e.g., another peptide or protein). As used herein, a “derivatized” antibody is an antibody that has been modified. Methods of derivatization can include, but are not limited to, the addition of a fluorescent moiety, a radionucleotide, a toxin, an enzyme or an affinity ligand such as biotin. For example, an antibody can be functionally linked to one or more other molecular entities, such as another antibody (e.g., a bispecific antibody or a diabody), a detectable agent, a cytotoxic agent, a pharmaceutical agent, and/or a protein or peptide that can mediate association of the antibody or antibody portion with another molecule (such as a streptavidin core region or a polyhistidine tag) by e.g., chemical coupling, genetic fusion, noncovalent association, or using other methods. One type of derivatized antibody can be produced by crosslinking two or more antibodies (of the same type or of different types, e.g., to create bispecific antibodies). Suitable crosslinkers can include those that are heterobifunctional, having two distinctly reactive groups separated by an appropriate spacer (e.g., m-maleimidobenzoyl-N-hydroxysuccinimide ester) or homobifunctional (e.g., disuccinimidyl suberate). Such linkers are available from Pierce Chemical Company, Rockford, Ill.

In some embodiments, antibodies can also be single domain antibodies. Single domain antibodies can include antibodies whose complementary determining regions are part of a single domain polypeptide. Non-limiting examples can include heavy chain antibodies, antibodies naturally devoid of light chains, single domain antibodies derived from conventional 4-chain antibodies, engineered antibodies and single domain scaffolds other than those derived from antibodies. Single domain antibodies can be any of the art, or any future single domain antibodies. Single domain antibodies can be derived from any species including, but not limited to mouse, human, camel, llama, fish, shark, goat, rabbit, and bovine. In some embodiments, a single domain antibody can be a naturally occurring single domain antibody known as heavy chain antibody devoid of light chains. Such single domain antibodies are disclosed in WO 94/04678, for example. In some embodiments, this variable domain derived from a heavy chain antibody naturally devoid of light chain is known herein as a VHH or nanobody. Such a VHH molecule can be derived from antibodies raised in Camelidae species, for example in camel, llama, dromedary, alpaca and guanaco. Other species besides Camelidae may produce heavy chain antibodies naturally devoid of light chain.

The VH and VL regions can be subdivided into regions of hypervariability, termed “complementarity determining regions” (CDR), interspersed with regions that are more conserved, termed “framework regions” (FR or FW). The extent of the framework region and CDRs has been precisely defined by a number of methods (see, Kabat, E. A., et al. (1991) Sequences of Proteins of Immunological Interest, Fifth Edition, U.S. Department of Health and Human Services, NIH Publication No. 91-3242; Chothia, C. et al. (1987) J. Mol. Biol. 196:901-917; and the AbM definition used by Oxford Molecular's AbM antibody modeling software. See, generally, e.g., Protein Sequence and Structure Analysis of Antibody Variable Domains. In: Antibody Engineering Lab Manual (Ed.: Duebel, S. and Kontermann, R., Springer-Verlag, Heidelberg). In some embodiments, CDRs can comprise amino acid sequences within antibody variable regions that confer antigen specificity and binding affinity. In some embodiments, antibodies can have three CDRs in each heavy chain variable region (VH-CDR1, VH-CDR2, and VH-CDR3) and three CDRs in each light chain variable region (VL-CDR1, VL-CDR2, and VL-CDR3). In some embodiments, boundaries of amino acid sequences of a given CDR can be determined using any of a number of known schemes, including those described by Kabat et al. (1991), “Sequences of Proteins of Immunological Interest,” 5th Ed. Public Health Service, National Institutes of Health, Bethesda, MD (“Kabat” numbering scheme), Al-Lazikani et al., (1997) JMB 273,927-948 (“Chothia” numbering scheme).

Antibodies described herein can be produced recombinantly, for example, using phase display or by using combinatorial methods. Phage display and combinatorial methods for generating antibodies are known in the art (as described in, e.g., Ladner et al. U.S. Pat. No. 5,223,409; Kang et al. International Publication No. WO 92/18619; Dower et al. International Publication No. WO 91/17271; Winter et al. International Publication WO 92/20791; Markland et al. International Publication No. WO 92/15679; Breitling et al. International Publication WO 93/01288; McCafferty et al. International Publication No. WO 92/01047; Garrard et al. International Publication No. WO 92/09690; Ladner et al. International Publication No. WO 90/02809; Fuchs et al. (1991) Bio/Technology 9:1370-1372; Hay et al. (1992) Hum Antibod Hybridomas 3:81-85; Huse et al. (1989) Science 246:1275-1281; Griffths et al. (1993) EMBO J 12:725-734; Hawkins et al. (1992) J Mol Biol 226:889-896; Clackson et al. (1991) Nature 352:624-628; Gram et al. (1992) PNAS 89:3576-3580; Garrad et al. (1991) Bio/Technology 9:1373-1377; Hoogenboom et al. (1991) Nuc Acid Res 19:4133-4137; and Barbas et al. (1991) PNAS 88:7978-7982, the contents of all of which are incorporated by reference herein).

In some embodiments, antibodies described herein can be fully human antibodies (e.g., antibodies made in a mouse which has been genetically engineered to produce antibodies from a human immunoglobulin sequence), or non-human antibodies, e.g., a rodent (mouse or rat), goat, primate (e.g., monkey), or camel antibodies. In some embodiments, non-human antibodies can be rodent antibodies (mouse or rat antibodies). Methods of producing rodent antibodies are known in the art.

In some embodiments, antibodies, antigen-binding fragments thereof, or functional fragments thereof described herein can be humanized antibodies or humanized antigen-binding fragments. As used herein, “humanized” antibodies refer to forms of non-human (e.g., murine) antibodies that are specific chimeric immunoglobulins, immunoglobulin chains, or fragments thereof that contain minimal sequence derived from non-human immunoglobulin. In some embodiments, humanized antibodies can be human immunoglobulins (recipient antibody) in which residues from a complementarity determining region (CDR) of the recipient are replaced by residues from a CDR of a non-human species (donor antibody) such as mouse, rat, or rabbit having the desired specificity, affinity, and biological activity. In some embodiments, humanized antibodies can have at least one or two, but generally all three, recipient CDRs (of heavy and or light immuoglobulin chains) replaced with a donor CDR. In some embodiments, antibodies may be replaced with at least a portion of a non-human CDR or only some of the CDRs may be replaced with non-human CDRs. In some embodiments, a minimal number of CDRs required for binding to the antigen can be replaced. In some embodiments, Fv framework region (FR) residues of the human immunoglobulin are replaced by corresponding non-human residues. Furthermore, humanized antibodies may comprise residues that are found neither in the recipient antibody nor in the imported CDR or framework sequences, but are included to further refine or optimize antibody performance. In general, a humanized antibody can comprise at least one, and typically two, variable domains, in which all or substantially all of the CDR regions correspond to those of a non-human immunoglobulin and all or substantially all of the FR regions are those of a human immunoglobulin consensus sequence. Humanized antibodies optimally also can comprise at least a portion of an immunoglobulin constant region or domain (Fc), typically that of a human immunoglobulin. Antibodies can have Fc regions modified as described in, for example, WO 99/58572. Other forms of humanized antibodies have one or more CDRs (one, two, three, four, five, or six) which are altered with respect to the original antibody, which are also termed one or more CDRs “derived from” one or more CDRs from the original antibody. Humanized antibodies can be produced, for example, by modeling the antibody variable domains and producing the antibodies using genetic engineering techniques, such as CDR grafting or CDR substitution, wherein one, two, or all CDRs of an immunoglobulin chain can be replaced. A description of various techniques for the production of humanized antibodies is found, for example, in U.S. Pat. No. 5,225,539; Morrison et al., (1984) Proc. Nat'l Acad. Sci. USA 81:6851-55; Whittle et al., (1987) Prot. Eng. 1:499-505; Co et al., (1990) J. Immunol. 148:1149-1154; Co et al., (1992) Proc. Nat'l Acad. Sci. USA 88:2869-2873; Carter et al., (1992) Proc. Nat'l Acad. Sci. USA 89:4285-4289; Routledge et al., (1991) Eur. J. Immunol. 21:2717-2725 and PCT Patent Publication Nos. WO 91/09967; WO 91/09968 and WO 92/113831. For example, human monoclonal antibodies can be generated using transgenic mice carrying the human immunoglobulin genes rather than the mouse system. Splenocytes from these transgenic mice immunized with the antigen of interest can be used to produce hybridomas that secrete human mAbs with specific affinities for epitopes from a human protein (see, e.g., Wood et al. International Application WO 91/00906, Kucherlapati et al. PCT publication WO 91/10741; Lonberg et al. International Application WO 92/03918; Kay et al. International Application 92/03917; Lonberg, N. et al. 1994 Nature 368:856-859; Green, L. L. et al. 1994 Nature Genet. 7:13-21; Morrison, S. L. et al. 1994 Proc. Natl. Acad. Sci. USA 81:6851-6855; Bruggeman et al. 1993 Year Immunol 7:33-40; Tuaillon et al. 1993 PNAS 90:3720-3724; Bruggeman et al. 1991 Eur J Immunol 21:1323-1326). In some embodiments, immunocompetent transgenic mice can be used. In some embodiments, immunocompetent transgenic mice can comprise human antibody heavy chains, human antibody light chains, or combinations thereof. In some embodiments, immunocompetent transgenic mice can comprise human antibody heavy chains, human antibody lamda light chains, human antibody kappa light chains or combinations thereof. In some embodiments, one or more specific amino acids can be substituted, deleted, or added in humanized antibodies. Criteria for selecting amino acids from the donor are described in U.S. Pat. No. 5,585,089, e.g., columns 12-16 of U.S. Pat. No. 5,585,089, e.g., columns 12-16 of U.S. Pat. No. 5,585,089, the contents of which are hereby incorporated by reference. Other techniques for humanizing antibodies are described in Padlan et al. EP 519596 A1, published on Dec. 23, 1992.

In some embodiments, antibodies, antigen-binding fragments thereof, or functional fragments thereof described herein can comprise a CDR-grafted scaffold domain. In some embodiments, the scaffold domain can be based on a fibronectin domain, e.g., fibronectin type III domain. In some embodiments, the overall fold of the fibronectin type III (Fn3) domain can be closely related to that of the smallest functional antibody fragment, the variable domain of the antibody heavy chain. There are three loops at the end of Fn3; the positions of BC, DE and FG loops approximately correspond to those of CDR1, 2 and 3 of the VH domain of an antibody. In some embodiments, Fn3 may not have disulfide bonds; and therefore, Fn3 can be stable under reducing conditions, unlike antibodies and their fragments (see, e.g., WO 98/56915; WO 01/64942; WO 00/34784). An Fn3 domain can be modified (e.g., using CDRs or hypervariable loops described herein) or varied, e.g., to select domains that bind to an antigen/marker/cell described herein. In some embodiments, a scaffold domain, e.g., a folded domain, can be based on an antibody, e.g., a “minibody” scaffold created by deleting three beta strands from a heavy chain variable domain of a monoclonal antibody (see, e.g., Tramontano et al., 1994, J Mol. Recognit. 7:9; and Martin et al., 1994, EMBO J. 13:5303-5309). In some embodiments, the minibody can be used to present two hypervariable loops. In some embodiments, the scaffold domain can be a V-like domain (see, e.g., Coia et al. WO 99/45110) or a domain derived from tendamistatin, which is a 74 residue, six-strand beta sheet sandwich held together by two disulfide bonds (see, e.g., McConnell and Hoess, 1995, J Mol. Biol. 250:460). For example, the loops of tendamistatin can be modified (e.g., using CDRs or hypervariable loops) or varied, e.g., to select domains that bind to a marker/antigen/cell described herein. Another exemplary scaffold domain is a beta-sandwich structure derived from the extracellular domain of CTLA-4 (see, e.g., WO 00/60070). Other exemplary scaffold domains can include, but are not limited to, T-cell receptors, MHC proteins, extracellular domains (e.g., fibronectin Type III repeats, EGF repeats), protease inhibitors (e.g., Kunitz domains, ecotin, BPTI, and so forth), TPR repeats; trifoil structures, zinc finger domains, DNA-binding proteins, particularly monomeric DNA binding proteins, RNA binding proteins, enzymes, e.g., proteases (particularly inactivated proteases), RNase, chaperones, e.g., thioredoxin, and heat shock proteins; and intracellular signaling domains (such as SH2 and SH3 domains). See, e.g., US20040009530 and U.S. Pat. No. 7,501,121, incorporated herein by reference. In some embodiments, a scaffold domain can be evaluated and chosen, e.g., by one or more of the following criteria: (1) amino acid sequence, (2) sequences of several homologous domains, (3) 3-dimensional structure, and/or (4) stability data over a range of pH, temperature, salinity, organic solvent, oxidant concentration. In some embodiments, the scaffold domain can be a small, stable protein domain, e.g., a protein of less than 100, 70, 50, 40 or 30 amino acids. The domain may include one or more disulfide bonds or may chelate a metal, e.g., zinc.

In some embodiments, antibodies, antigen-binding fragments thereof, or functional fragments thereof described herein can comprise variable regions, or a portion thereof, e.g., CDRs, generated in a non-human organism (e.g., a rat or mouse). In some embodiments, antibodies, antigen-binding fragments thereof, or functional fragments thereof described herein can be chimeric, CDR-grafted, or humanized antibodies. In some embodiments, antibodies, antigen-binding fragments thereof, or functional fragments thereof described herein can be generated in a non-human organism and modified. For example, antibodies, antigen-binding fragments thereof, or functional fragments thereof generated in a non-human organism (e.g., a rat or mouse) can be modified in the variable framework or constant region, to decrease antigenicity and/or immunogenicity in humans. In some embodiments, chimeric antibodies can be produced by recombinant DNA techniques known in the art (see, Robinson et al., International Patent Publication PCT/US86/02269; Akira, et al., European Patent Application 184,187; Taniguchi, M., European Patent Application 171,496; Morrison et al., European Patent Application 173,494; Neuberger et al., International Application WO 86/01533; Cabilly et al. U.S. Pat. No. 4,816,567; Cabilly et al., European Patent Application 125,023; Better et al. (1988 Science 240:1041-1043); Liu et al. (1987) PNAS 84:3439-3443; Liu et al., 1987, J. Immunol. 139:3521-3526; Sun et al. (1987) PNAS 84:214-218; Nishimura et al., 1987, Canc. Res. 47:999-1005; Wood et al. (1985) Nature 314:446-449; and Shaw et al., 1988, J. Natl Cancer Inst. 80:1553-1559).

In some embodiments, antibodies, antigen-binding fragments thereof, or functional fragments thereof described herein that can selectively binds to an antigen of a target protein or an epitope on an antigen of a target protein. In some embodiments, the target can comprise an erythropoietin (EPO) protein, an EPO receptor subunit of a homo-EPOR or a hetero-EPOR, a CD131 subunit of a hetero-EPOR, or a combination thereof. In some embodiments, the target can comprise a homo-EPOR complex comprising two EPOR subunits, or a hetero-EPOR complex comprising an EPOR subunit and a CD131 subunit. For example, antibodies, antigen-binding fragments thereof, or functional fragments thereof described herein can selectively binds to a hetero-EPOR complex comprising an EPOR subunit and a CD131 subunit. In this embodiment, antibodies, antigen-binding fragments thereof, or functional fragments thereof described herein can bind to an EPOR subunit and/or a CD131 subunit of a hetero-EPOR complex. In some embodiments, antibodies, antigen-binding fragments thereof, or functional fragments thereof described herein can be non-naturally occurring. In some embodiments, antibodies, antigen-binding fragments thereof, or functional fragments thereof described herein can be isolated and/or purified. In some embodiments, antibodies, antigen-binding fragments thereof, or functional fragments thereof described herein can be used in in vitro assays (e.g., binding assays, functional assays, etc.).

In some embodiments, antibodies or functional fragments thereof described herein can bind to a target and can act as an antagonist. In one example, an anti-EPO antibody can bind an EPO protein and can prevent, inhibit, or reduce formation of a complex between an EPO protein and an EPOR complex. In another example, an anti-EPOR antibody can bind an EPO receptor subunit and can prevent, inhibit, or reduce complex formation of an EPOR complex or complex formation between an EPO protein and an EPOR complex. In some embodiments, preventing, inhibiting, or reducing complex formation of a homo-EPOR or complex formation between an EPO protein and an EPOR complex can lead to prevention, inhibition, or reduction of EPOR activation or function. In some embodiments, an anti-EPO antibody can bind an EPO protein and inhibit or decrease the level of an activity of an EPOR without affecting binding of the EPO protein to the EPOR. In some embodiments, an anti-EPOR antibody can bind to an EPOR and inhibit or decrease the level of an activity of the EPOR without affecting the complex formation of the EPOR or complex formation between an EPO protein and the EPOR.

In some embodiments, an EPOR activity can include, but are not limited to, phosphorylation of an intracellular domain of an EPOR, Janus tyrosine kinase 2 (Jak2), or Signal transducer and activator of transcription 5 (Stat5). In some embodiments, an EPOR activity can include, but are not limited to, activation of Jak2, Jak2 pathway, Stat5 pathway, mitogen-activated protein kinase (MAPK), MAPK pathway, extracellular signal-regulated kinase (ERK), ERK pathway, phosphatidylinositol 3-kinase (PI3K), PI3K pathway, v-Akt Murine Thymoma Viral Oncogene/Protein Kinase-B (Akt/PKB), Akt/PKB pathway, Mammalian Target of rapamycin (mTOR), or mTOR pathway. In some embodiments, antibodies or functional fragments thereof described herein can inhibit activation or phosphorylation of homo-EPOR, hetero-EPOR, Jak2, Stat5, MAPK, ERK, PI3K, Akt/PKB, or mTOR. In some embodiments, antibodies or functional fragments thereof described herein can inhibit activation of Jak2, Jak2 pathway, Stat5, Stat5 pathway, MAPK, MAPK pathway, ERK, ERK pathway, PI3K, PIK3 pathway, Akt/PKB, Akt/PKB pathway, mTOR, or mTOR pathway. In some embodiments, antibodies or functional fragments thereof described herein can promote activation or phosphorylation of EPOR, Jak2, Stat5, or mTOR. In some embodiments, antibodies or functional fragments thereof described herein can promote activation of Jak2, Jak2 pathway, Stat5, Stat5 pathway, MAPK, MAPK pathway, ERK, ERK pathway, PI3K, PIK3 pathway, Akt/PKB, Akt/PKB pathway, mTOR, or mTOR pathway. In some embodiments, antibodies or functional fragments thereof described herein may not affect activation or phosphorylation of EPOR, Jak2, Stat5, MAPK, ERK, PI3K, Akt/PKB, or mTOR. In some embodiments, antibodies or functional fragments thereof described herein may not affect activation of Jak2, Jak2 pathway, Stat5, Stat5 pathway, MAPK, MAPK pathway, ERK, ERK pathway, PI3K, PIK3 pathway, Akt/PKB, Akt/PKB pathway, mTOR, or mTOR pathway. In some embodiments, activation or phosphorylation of EPOR, Jak2, Stat5, MAPK, ERK, PI3K, Akt/PKB, or mTOR can be measured using any methods known in the art. Examples of methods to measure Jak2, Stat5, MAPK, ERK, PI3K, Akt/PKB, or mTOR activation level include, but are not limited to, western blotting, a flow cytometry assay, a cell proliferation assay, an apoptosis assay, or enzyme-linked immunosorbent assay (ELISA).

In some embodiments, anti-EPO antibodies, anti-EPOR antibodies, anti-CD131 antibodies, or functional fragments thereof described herein can bind to a target and can act as antagonists for an EPOR comprising an EPOR subunit and a CD131 subunit. In some embodiments, anti-EPO antibodies, anti-EPOR antibodies, anti-CD131 antibodies, or functional fragments thereof described herein can be antagonists for an EPOR comprising an EPOR subunit and a CD131 subunit and can selectively bind the EPOR and have binding specificity or selectivity for the EPOR. In some embodiments, anti-EPO antibodies, anti-EPOR antibodies, anti-CD131 antibodies, or functional fragments thereof can be antagonists for an EPOR comprising an EPOR subunit and a CD131 subunit and can have a higher binding affinity, specificity, or selectivity to the EPOR than to an EPOR comprising two EPOR subunits. For example, anti-EPO antibodies, anti-EPOR antibodies, anti-CD131 antibodies, or functional fragments thereof described herein can be antagonists for an EPOR comprising an EPOR subunit and a CD131 subunit and can have an EPOR binding affinity, specificity, or selectivity that is higher than a binding affinity, specificity, or selectivity for an EPOR comprising two EPOR subunits. In some embodiments, anti-EPO antibodies, anti-EPOR antibodies, anti-CD131 antibodies, or functional fragments thereof described herein can be antagonists for an EPOR comprising an EPOR subunit and a CD131 subunit and have an EPOR binding affinity, specificity, or selectivity that is at least about 1%, at least about 2%, at least about 3%, at least about 4%, at least about 5%, at least about 6%, at least about 7%, at least about 8%, at least about 9%, at least about 10%, at least about 11%, at least about 12%, at least about 13%, at least about 14%, at least about 15%, at least about 16%, at least about 17%, at least about 18%, at least about 19%, at least about 20%, at least about 21%, at least about 22%, at least about 23%, at least about 24%, at least about 25%, at least about 26%, at least about 27%, at least about 28%, at least about 29%, at least about 30%, at least about 31%, at least about 32%, at least about 33%, at least about 34%, at least about 35%, at least about 36%, at least about 37%, at least about 38%, at least about 39%, at least about 40%, at least about 41%, at least about 42%, at least about 43%, at least about 44%, at least about 45%, at least about 46%, at least about 47%, at least about 48%, at least about 49%, at least about 50%, at least about 55%, at least about 60%, at least about 65%, at least about 70%, at least about 75%, at least about 80%, at least about 85%, at least about 90%, at least about 95%, or at least about 99% higher than a binding affinity, specificity or selectivity for an EPOR comprising two EPOR subunits.

In some embodiments, anti-EPO antibodies, anti-EPOR antibodies, or functional fragments thereof described herein can bind to a target and can act as antagonists for an EPOR comprising two EPOR subunits. In some embodiments, anti-EPO antibodies, anti-EPOR antibodies, or functional fragments thereof described herein can be antagonists for an EPOR comprising two EPOR subunits and can selectively bind to homo-EPOR. In some embodiments, anti-EPO antibodies, anti-EPOR antibodies, or functional fragments thereof can be antagonists for an EPOR comprising two EPOR subunits and can have a higher binding affinity to the EPOR than to an EPOR comprising an EPOR subunit and a CD131 subunit. For example, anti-EPO antibodies, anti-EPOR antibodies, or functional fragments thereof described herein can be antagonists for an EPOR comprising two EPOR subunits and can have an EPOR binding affinity, specificity, or selectivity that is higher than a binding affinity, specificity, or selectivity for an EPOR comprising an EPOR subunit and a CD131 subunit. In some embodiments, anti-EPO antibodies, anti-EPOR antibodies, or functional fragments thereof described herein can be antagonists for an EPOR comprising two EPOR subunits and have an EPOR binding affinity, specificity, or selectivity that is at least about 1%, at least about 2%, at least about 3%, at least about 4%, at least about 5%, at least about 6%, at least about 7%, at least about 8%, at least about 9%, at least about 10%, at least about 11%, at least about 12%, at least about 13%, at least about 14%, at least about 15%, at least about 16%, at least about 17%, at least about 18%, at least about 19%, at least about 20%, at least about 21%, at least about 22%, at least about 23%, at least about 24%, at least about 25%, at least about 26%, at least about 27%, at least about 28%, at least about 29%, at least about 30%, at least about 31%, at least about 32%, at least about 33%, at least about 34%, at least about 35%, at least about 36%, at least about 37%, at least about 38%, at least about 39%, at least about 40%, at least about 41%, at least about 42%, at least about 43%, at least about 44%, at least about 45%, at least about 46%, at least about 47%, at least about 48%, at least about 49%, at least about 50%, at least about 55%, at least about 60%, at least about 65%, at least about 70%, at least about 75%, at least about 80%, at least about 85%, at least about 90%, at least about 95%, or at least about 99% higher than a binding affinity, specificity or selectivity for an EPOR comprising an EPOR subunit and a CD131.

In some aspects, antibodies described herein have specificity for EPO, hetero-EPOR, or homo-EPOR and include all the forms described above. The antibody can be engineered for use in a particular organism. The organism can be a human, canine, or a commercially valuable livestock, such as, for example, pigs, horses, dogs, cats, chickens, or other birds. Such engineering of the antibody can include, for example, CDR splicing, humanization, humaneering, chimerization, or isolating human (or other organism) antibodies using any of the repertoire technologies or monoclonal technologies known in the art.

Certain examples of antibodies with alternative scaffolds can include, but are not limited to, nanobodies, affibodies, microbodies, evibodies, and domain antibodies. Certain examples of alternative scaffolds useful for creating antibodies can include, but are not limited to, single domain antibodies from camelids; protease inhibitors; human serum transferrin; CTLA-4; fibronectin, including, but not limited to, the fibronectin type III domain; C-type lectin-like domains; lipocalin family proteins; ankyrin repeat proteins; the Z-domain of Protein A; gamma-crystallin; Tendamistat; Neocarzinostatin; CBM4-2; the T-cell receptor; Im9; designed AR proteins; designed TPR proteins; zinc finger domains; pVIII; Avian Pancreatic Polypeptide; GCN4; WW domains; Src Homology 3 (SH3) domains; Src Homology 2 (SH2) domains; PDZ domains; TEM-1 beta-lactamase; GFP; Thioredoxin; Staphylcoccal nuclease; PHD-finger domains; CI-2; BPTI; APPI; HPSTI; Ecotin; LACI-D1; LDTI; MTI-II; scorpion toxins; Insect Defensin A Peptide; EETI-II; Min-23; CBD; PBP; Cytochrome b₅₆₂; Transferrin; LDL Receptor Domain A; and ubiquitin. Certain examples of alternative scaffolds are discussed in Hey et al., “Artificial, non-antibody binding proteins for pharmaceutical and industrial applications” Trends in Biotechnology, 23:514-22 (2005) and Binz et al., “Engineering novel binding proteins from nonimmunoglobulin domains” Nature Biotechnology, 23:1257-68 (2005), both of which are incorporated by reference in their entirety for all purposes.

A bispecific or bifunctional antibody can comprise two different heavy/light chain pairs and two different binding sites. Bispecific antibodies may be produced by a variety of methods including, but not limited to, fusion of hybridomas or linking of Fab′ fragments. See, e.g., Songsivilai & Lachmann Clin. Exp. Immunol 79:315-321 (1990), Kostelny et al. J. Immunol. 148:1547-1553 (1992), which is incorporated by reference in its entirety for all purposes.

Bispecific antibody molecules can be classified into five different structural groups: (i) bispecific immunoglobulin G (BsIgG); (ii) IgG appended with an additional antigen-binding moiety; (iii) bispecific antibody fragments; (iv) bispecific fusion proteins; and (v) bispecific antibody conjugates. BsIgG is a format that is monovalent for each antigen. Exemplary BsIgG formats include but are not limited to crossMab, DAF (two-in-one), DAF (four-in-one), DutaMab, DT-IgG, knobs-in-holes common LC, knobs-in-holes assembly, charge pair, Fab-arm exchange, SEEDbody, triomab, LUZ-Y, Fcab, KA-body, orthogonal Fab. See Spiess et al. Mol. Immunol. 67 (2015):95-106. Exemplary BsIgGs include catumaxomab (Fresenius Biotech, Trion Pharma, Neopharm), which contains an anti-CD3 arm and an anti-EpCAM arm; and ertumaxomab (Neovii Biotech, Fresenius Biotech), which targets CD3 and HER2. In some embodiments, BsIgG comprises heavy chains that are engineered for heterodimerization. For example, heavy chains can be engineered for heterodimerization using a “knobs-into-holes” strategy, a SEED platform, a common heavy chain (e.g., in κλ-bodies), and use of heterodimeric Fc regions. See Spiess et al. Mol. Immunol. 67 (2015):95-106. Strategies that have been used to avoid heavy chain pairing of homodimers in BsIgG include knobs-in-holes, duobody, azymetric, charge pair, HA-TF, SEEDbody, and differential protein A affinity. See Id. BsIgG can be produced by separate expression of the component antibodies in different host cells and subsequent purification/assembly into a BsIgG. BsIgG can also be produced by expression of the component antibodies in a single host cell. BsIgG can be purified using affinity chromatography, e.g., using protein A and sequential pH elution. IgG appended with an additional antigen-binding moiety is another format of bispecific antibody molecules. For example, monospecific IgG can be engineered to have bispecificity by appending an additional antigen-binding unit onto the monospecific IgG, e.g., at the N- or C-terminus of either the heavy or light chain. Exemplary additional antigen-binding units include single domain antibodies (e.g., variable heavy chain or variable light chain), engineered protein scaffolds, and paired antibody variable domains (e.g., single chain variable fragments or variable fragments). See Id. Examples of appended IgG formats include dual variable domain IgG (DVD-Ig), IgG (H)-scFv, scFv-(H) IgG, IgG (L)-scFv, scFv-(L) IgG, IgG (L,H)-Fv, IgG (H)-V, V(H)-IgG, IgG (L)-V, V(L)-IgG, KIH IgG-scFab, 2scFv-IgG, IgG-2scFv, scFv4-Ig, zybody, and DVI-IgG (four-in-one). See Spiess et al. Mol. Immunol. 67(2015):95-106. An example of an IgG-scFv is MM-141 (Merrimack Pharmaceuticals), which binds IGF-1R and HER3. Examples of DVD-Ig include ABT-981 (Abb Vie), which binds IL-1α and IL-1β; and ABT-122 (AbbVie), which binds TNF and IL-17A.

Bispecific antibody fragments (BsAb) are a format of bispecific antibody molecules that lack some or all of the antibody constant domains. For example, some BsAb lack an Fc region. In some embodiments, bispecific antibody fragments include heavy and light chain regions that are connected by a peptide linker that permits efficient expression of the BsAb in a single host cell. Exemplary bispecific antibody fragments include but are not limited to nanobody, nanobody-HAS, BiTE, Diabody, DART, TandAb, scDiabody, scDiabody-CH3, Diabody-CH3, triple body, miniantibody, minibody, TriBi minibody, scFv-CH3 KIH, Fab-scFv, scFv-CH-CL-scFv, F(ab′)2, F(ab′)2-scFv2, scFv-KIH, Fab-scFv-Fc, tetravalent HCAb, scDiabody-Fc, Diabody-Fc, tandem scFv-Fc, and intrabody. For example, the BiTE format comprises tandem scFvs, where the component scFvs bind to CD3 on T cells and a surface antigen on cancer cells. Bispecific fusion proteins include antibody fragments linked to other proteins, e.g., to add additional specificity and/or functionality. An example of a bispecific fusion protein is an immTAC, which comprises an anti-CD3 scFv linked to an affinity-matured T-cell receptor that recognizes HLA-presented peptides. In some embodiments, the dock-and-lock (DNL) method can be used to generate bispecific antibody molecules with higher valency. Also, fusions to albumin binding proteins or human serum albumin can be extend the serum half-life of antibody fragments. In some embodiments, chemical conjugation, e.g., chemical conjugation of antibodies and/or antibody fragments, can be used to create BsAb molecules. An exemplary bispecific antibody conjugate includes the CovX-body format, in which a low molecular weight drug is conjugated site-specifically to a single reactive lysine in each Fab arm or an antibody or fragment thereof. In some embodiments, the conjugation improves the serum half-life of the low molecular weight drug. An exemplary CovX-body is CVX-241 (NCT01004822), which comprises an antibody conjugated to two short peptides inhibiting either VEGF or Ang2. In some instances, bispecific antibodies can further comprise a linker. In some instances, bispecific antibodies can further comprise a Fc domain. The Fc domain can be, for example, a human IgG1 Fc domain. The Fc domain can comprise a knob-in-hole. In some instances, bispecific antibodies can further comprise a linker and an Fc domain. In some embodiments, a linker can be a peptide linker. Non-limiting examples of peptide linkers can include (GS)ₙ (SEQ ID NO: 73), (GGS)ₙ (SEQ ID NO: 74), (GGGS)ₙ (SEQ ID NO: 75), (GGSG)ₙ (SEQ ID NO: 76), (GGSGG)ₙ (SEQ ID NO: 77), or (GGGGS)ₙ (SEQ ID NO: 78), wherein n can be 1, 2, 3, 4, 5, 6, 7, 8, 9, or 10. For example, a linking peptide can be (GGGGS)₃ (SEQ ID NO: 79) or (GGGGS)₄ (SEQ ID NO: 80). Linkers described herein can be used for multispecific antibodies. In this embodiment, multispecific antibodies can have more than one linker. In this embodiment, the linker can be the same. Alternatively, the linkers can be different.

The antibody molecules can be produced by recombinant expression, e.g., of at least one or more component, in a host system. Exemplary host systems include eukaryotic cells (e.g., mammalian cells, e.g., CHO or HEK293 cells, or insect cells, e.g., SF9 or S2 cells) and prokaryotic cells (e.g., E. coli). Bispecific antibody molecules can be produced by separate expression of the components in different host cells and subsequent purification/assembly. Alternatively, the antibody molecules can be produced by expression of the components in a single host cell. Purification of bispecific antibody molecules can be performed by various methods such as affinity chromatography, e.g., using protein A and sequential pH elution. In other embodiments, affinity tags can be used for purification, e.g., histidine-containing tag, myc tag, or streptavidin tag.

In an aspect, an antibody may be part of a conjugate molecule comprising all or part of the antibody and a prodrug. The term “prodrug” refers to a precursor or derivative form of a pharmaceutically active substance. A prodrug can be less cytotoxic to cells compared to the parent drug and capable of being enzymatically activated or converted into the more active cytotoxic parent form. Exemplary prodrugs can include, but are not limited to, phosphate-containing prodrugs, thiophosphate-containing prodrugs, sulfate-containing prodrugs, peptide-containing prodrugs, D-amino acid-modified prodrugs, glycosylated prodrugs, beta-lactam-containing prodrugs, optionally substituted phenoxyacetamide-containing prodrugs and optionally substituted phenylacetamide-containing prodrugs, 5-fluorocytosine and other 5-fluorouridine prodrugs which can be converted into a more active cytotoxic free drug. Examples of cytotoxic drugs that can be derivatized into a prodrug form can include, but are not limited to, those cytotoxic agents described above. See, e.g., U.S. Pat. No. 6,702,705.

In some aspect, an anti-EPOR, anti-CD131, or anti-EPO antibody can comprise an antigen-binding domain or an antigen-binding fragment. In some embodiments, an antigen-binding domain or an antigen-binding fragment can comprise a heavy chain variable region (VH), a light chain variable region (VL), or a combination thereof. In some embodiments, a heavy chain variable region (VH) can comprise an amino acid sequence with at least about 100%, 99.9%, 99.8%, 99.7%, 99.6%, 99.5%, 99.4%, 99.3%, 99.2%, 99.1%, 99%, 98%, 97%, 96%, 95%, 94%, 93%, 92%, 91%, 90%, 85%, 80%, 75%, 70%, 65%, 60%, 55%, 50%, 45%, 40%, 35%, 30%, 25%, or at least about 20% sequence identity or sequence similarity to any one of VH sequences listed in Table 1-1, 4, 6, 8, 13, 15, or 17. In some embodiments, a VH can comprise an amino acid sequence with at most about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 95.5%, 96%, 96.5%, 97%, 97.5%, 98%, 98.5%, 99%, or at most about 99.5% sequence identity or sequence similarity to any one of VH sequences listed in Table 1-1, 4, 6, 8, 13, 15, or 17. In some embodiments, a VH can comprise an amino acid sequence with about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 95.5%, 96%, 96.5%, 97%, 97.5%, 98%, 98.5%, 99%, 99.5%, 99.6%, 99.7%, 99.8%, 99.9%, or about 100% sequence identity or sequence similarity to any one of VH sequences listed in Table 1-1, 4, 6, 8, 13, 15, or 17. In some embodiments, a VH can comprise any one of VH sequences listed in Table 1-1, 4, 6, 8, 13, 15, or 17, or a variant thereof. In some embodiments, a VH can comprise an amino acid sequence with at least about 100%, 99.9%, 99.8%, 99.7%, 99.6%, 99.5%, 99.4%, 99.3%, 99.2%, 99.1%, 99%, 98%, 97%, 96%, 95%, 94%, 93%, 92%, 91%, 90%, 85%, 80%, 75%, 70%, 65%, 60%, 55%, 50%, 45%, 40%, 35%, 30%, 25%, or at least about 20% sequence identity or sequence similarity to any one of SEQ ID NOs: 3, 147-334, 523-651, 781-916, 3824-3921, 4020-4050, 4088-4245, or 5616-5803. In some embodiments, a VH can comprise an amino acid sequence with at most about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 95.5%, 96%, 96.5%, 97%, 97.5%, 98%, 98.5%, 99%, or at most about 99.5% sequence identity or sequence similarity to any one of SEQ ID NOs: 3, 147-334, 523-651, 781-916, 3824-3921, 4020-4050, 4088-4245, or 5616-5803. In some embodiments, a VH can comprise an amino acid sequence with about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 95.5%, 96%, 96.5%, 97%, 97.5%, 98%, 98.5%, 99%, 99.5%, 99.6%, 99.7%, 99.8%, 99.9%, or about 100% sequence identity or sequence similarity to any one of SEQ ID NOs: 3, 147-334, 523-651, 781-916, 3824-3921, 4020-4050, 4088-4245, or 5616-5803. In some embodiments, a VH can comprise an amino acid sequence of any one of SEQ ID NOs: 3, 147-334, 523-651, 781-916, 3824-3921, 4020-4050, 4088-4245, or 5616-5803, or a variant thereof. In some embodiments, a variant can comprise a conservatively modified or substituted amino acid sequence variant.

In some embodiments, a light chain variable region (VL) can comprise an amino acid sequence with at least about 100%, 99.9%, 99.8%, 99.7%, 99.6%, 99.5%, 99.4%, 99.3%, 99.2%, 99.1%, 99%, 98%, 97%, 96%, 95%, 94%, 93%, 92%, 91%, 90%, 85%, 80%, 75%, 70%, 65%, 60%, 55%, 50%, 45%, 40%, 35%, 30%, 25%, or at least about 20% sequence identity or sequence similarity to any one of VL sequences listed in Table 1-1, 4, 6, 8, 13, 15, or 17. In some embodiments, a VL can comprise an amino acid sequence with at most about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 95.5%, 96%, 96.5%, 97%, 97.5%, 98%, 98.5%, 99%, or at most about 99.5% sequence identity or sequence similarity to any one of VL sequences listed in Table 1-1, 4, 6, 8, 13, 15, or 17. In some embodiments, a VL can comprise an amino acid sequence with about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 95.5%, 96%, 96.5%, 97%, 97.5%, 98%, 98.5%, 99%, 99.5%, 99.6%, 99.7%, 99.8%, 99.9%, or about 100% sequence identity or sequence similarity to any one of VL sequences listed in Table 1-1, 4, 6, 8, 13, 15, or 17. In some embodiments, a VL can comprise any one of VL sequences listed in Table 1-1, 4, 6, 8, 13, 15, or 17, or a variant thereof. In some embodiments, a light chain variable region (VL) can comprise an amino acid sequence with at least about 100%, 99.9%, 99.8%, 99.7%, 99.6%, 99.5%, 99.4%, 99.3%, 99.2%, 99.1%, 99%, 98%, 97%, 96%, 95%, 94%, 93%, 92%, 91%, 90%, 85%, 80%, 75%, 70%, 65%, 60%, 55%, 50%, 45%, 40%, 35%, 30%, 25%, or at least about 20% sequence identity or sequence similarity to any one of SEQ ID NOs: 4, 335-522, 652-780, 917-1052, 3922-4019, 4051-4087, 4246-4403, or 5804-5991. In some embodiments, a VL can comprise an amino acid sequence with at most about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 95.5%, 96%, 96.5%, 97%, 97.5%, 98%, 98.5%, 99%, or at most about 99.5% sequence identity or sequence similarity to any one of SEQ ID NOs: 4, 335-522, 652-780, 917-1052, 3922-4019, 4051-4087, 4246-4403, or 5804-5991. In some embodiments, a VL can comprise an amino acid sequence with about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 95.5%, 96%, 96.5%, 97%, 97.5%, 98%, 98.5%, 99%, 99.5%, 99.6%, 99.7%, 99.8%, 99.9%, or about 100% sequence identity or sequence similarity to any one of SEQ ID NOs: 4, 335-522, 652-780, 917-1052, 3922-4019, 4051-4087, 4246-4403, or 5804-5991. In some embodiments, a VL can comprise an amino acid sequence of any one of SEQ ID NOs: 4, 335-522, 652-780, 917-1052, 3922-4019, 4051-4087, 4246-4403, or 5804-5991, or a variant thereof. In some embodiments, a variant can comprise a conservatively modified or substituted amino acid sequence variant.

In some embodiments, an anti-EPOR, anti-CD131, or anti-EPO antibody can comprise a VH comprising an amino acid sequence with at least about 100%, 99.9%, 99.8%, 99.7%, 99.6%, 99.5%, 99.4%, 99.3%, 99.2%, 99.1%, 99%, 98%, 97%, 96%, 95%, 94%, 93%, 92%, 91%, 90%, 85%, 80%, 75%, 70%, 65%, 60%, 55%, 50%, 45%, 40%, 35%, 30%, 25%, or at least about 20% sequence identity or sequence similarity to any one of VH sequences listed in Table 1-1, 4, 6, 8, 13, 15, or 17 and a VL comprising an amino acid sequence with at least about 100%, 99.9%, 99.8%, 99.7%, 99.6%, 99.5%, 99.4%, 99.3%, 99.2%, 99.1%, 99%, 98%, 97%, 96%, 95%, 94%, 93%, 92%, 91%, 90%, 85%, 80%, 75%, 70%, 65%, 60%, 55%, 50%, 45%, 40%, 35%, 30%, 25%, or at least about 20% sequence identity or sequence similarity to any one of VL sequences listed in Table 1-1, 4, 6, 8, 13, 15, or 17. In some embodiments, an anti-EPOR, anti-CD131, or anti-EPO antibody can comprise a VH comprising an amino acid sequence with at most about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 95.5%, 96%, 96.5%, 97%, 97.5%, 98%, 98.5%, 99%, or at most about 99.5% sequence identity or sequence similarity to any one of VH sequences listed in Table 1-1, 4, 6, 8, 13, 15, or 17 and a VL comprising an amino acid sequence with at most about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 95.5%, 96%, 96.5%, 97%, 97.5%, 98%, 98.5%, 99%, or at most about 99.5% sequence identity or sequence similarity to any one of VL sequences listed in Table 1-1, 4, 6, 8, 13, 15, or 17. In some embodiments, an anti-EPOR, anti-CD131, or anti-EPO antibody can comprise a VH comprising an amino acid sequence with about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 95.5%, 96%, 96.5%, 97%, 97.5%, 98%, 98.5%, 99%, 99.5%, 99.6%, 99.7%, 99.8%, 99.9%, or about 100% sequence identity or sequence similarity to any one of VH sequences listed in Table 1-1, 4, 6, 8, 13, 15, or 17 and a VL comprising an amino acid sequence with about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 95.5%, 96%, 96.5%, 97%, 97.5%, 98%, 98.5%, 99%, 99.5%, 99.6%, 99.7%, 99.8%, 99.9%, or about 100% sequence identity or sequence similarity to any one of VL sequences listed in Table 1-1, 4, 6, 8, 13, 15, or 17. In some embodiments, an anti-EPOR, anti-CD131, or anti-EPO antibody can comprise a VH comprising an amino acid sequence of any one of VH sequences listed in Table 1-1, 4, 6, 8, 13, 15, or 17, or a variant thereof and a VL comprising an amino acid sequence of any one of VL sequences listed in Table 1-1, 4, 6, 8, 13, 15, or 17, or a variant thereof. In some embodiments, a variant can comprise a conservatively modified or substituted amino acid sequence variant.

In some embodiments, an anti-EPOR, anti-CD131, or anti-EPO antibody can comprise a VH comprising an amino acid sequence with at least about 100%, 99.9%, 99.8%, 99.7%, 99.6%, 99.5%, 99.4%, 99.3%, 99.2%, 99.1%, 99%, 98%, 97%, 96%, 95%, 94%, 93%, 92%, 91%, 90%, 85%, 80%, 75%, 70%, 65%, 60%, 55%, 50%, 45%, 40%, 35%, 30%, 25%, or at least about 20% sequence identity or sequence similarity to any one of SEQ ID NOs: 3, 147-334, 523-651, 781-916, 3824-3921, 4020-4050, 4088-4245, or 5616-5803 and a VL comprising an amino acid sequence with at least about 100%, 99.9%, 99.8%, 99.7%, 99.6%, 99.5%, 99.4%, 99.3%, 99.2%, 99.1%, 99%, 98%, 97%, 96%, 95%, 94%, 93%, 92%, 91%, 90%, 85%, 80%, 75%, 70%, 65%, 60%, 55%, 50%, 45%, 40%, 35%, 30%, 25%, or at least about 20% sequence identity or sequence similarity to any one of SEQ ID NOs: 4, 335-522, 652-780, 917-1052, 3922-4019, 4051-4087, 4246-4403, or 5804-5991. In some embodiments, an anti-EPOR, anti-CD131, or anti-EPO antibody can comprise a VH comprising an amino acid sequence with at most about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 95.5%, 96%, 96.5%, 97%, 97.5%, 98%, 98.5%, 99%, or at most about 99.5% sequence identity or sequence similarity to any one of SEQ ID NOs: 3, 147-334, 523-651, 781-916, 3824-3921, 4020-4050, 4088-4245, or 5616-5803 and a VL comprising an amino acid sequence with at most about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 95.5%, 96%, 96.5%, 97%, 97.5%, 98%, 98.5%, 99%, or at most about 99.5% sequence identity or sequence similarity to any one of SEQ ID NOs: 4, 335-522, 652-780, 917-1052, 3922-4019, 4051-4087, 4246-4403, or 5804-5991. In some embodiments, an anti-EPOR, anti-CD131, or anti-EPO antibody can comprise a VH comprising an amino acid sequence with about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 95.5%, 96%, 96.5%, 97%, 97.5%, 98%, 98.5%, 99%, 99.5%, 99.6%, 99.7%, 99.8%, 99.9%, or about 100% sequence identity or sequence similarity to any one of SEQ ID NOs: 3, 147-334, 523-651, 781-916, 3824-3921, 4020-4050, 4088-4245, or 5616-5803 and a VL comprising an amino acid sequence with about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 95.5%, 96%, 96.5%, 97%, 97.5%, 98%, 98.5%, 99%, 99.5%, 99.6%, 99.7%, 99.8%, 99.9%, or about 100% sequence identity or sequence similarity to any one of SEQ ID NOs: 4, 335-522, 652-780, 917-1052, 3922-4019, 4051-4087, 4246-4403, or 5804-5991. In some embodiments, an anti-EPOR, anti-CD131, or anti-EPO antibody can comprise a VH comprising an amino acid sequence of any one of SEQ ID NOs: 3, 147-334, 523-651, 781-916, 3824-3921, 4020-4050, 4088-4245, or 5616-5803, or a variant thereof and a VL comprising an amino acid sequence of any one of SEQ ID NOs: 4, 335-522, 652-780, 917-1052, 3922-4019, 4051-4087, 4246-4403, or 5804-5991, or a variant thereof. In some embodiments, a variant can comprise a conservatively modified or substituted amino acid sequence variant.

In some embodiments, a VH can comprise a VH complementarity determining region 1 (VH-CDR1), a VH-CDR2, or a VH-CDR3. In some embodiments, a VH can comprise a VH complementarity determining region 1 (VH-CDR1), a VH-CDR2, and a VH-CDR3. In some embodiments, VH-CDR1, VH-CDR2, and/or VH-CDR3 can be defined according to Kabat, Chothia, IMGT, AbM, or Contact.

In some embodiments, a VH-CDR1 can comprise an amino acid sequence with at least about 100%, 99.9%, 99.8%, 99.7%, 99.6%, 99.5%, 99.4%, 99.3%, 99.2%, 99.1%, 99%, 98%, 97%, 96%, 95%, 94%, 93%, 92%, 91%, 90%, 85%, 80%, 75%, 70%, 65%, 60%, 55%, 50%, 45%, 40%, 35%, 30%, 25%, or at least about 20% sequence identity or sequence similarity to any one of VH-CDR1 sequences listed in Table 5, 7, 9, or 18. In some embodiments, a VH-CDR1 can comprise an amino acid sequence with at most about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 95.5%, 96%, 96.5%, 97%, 97.5%, 98%, 98.5%, 99%, or at most about 99.5% sequence identity or sequence similarity to any one of VH-CDR1 sequences listed in Table 5, 7, 9, or 18. In some embodiments, a VH-CDR1 can comprise an amino acid sequence with about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 95.5%, 96%, 96.5%, 97%, 97.5%, 98%, 98.5%, 99%, 99.5%, 99.6%, 99.7%, 99.8%, 99.9%, or about 100% sequence identity or sequence similarity to any one of VH-CDR1 sequences listed in Table 5, 7, 9, or 18. In some embodiments, a VH-CDR1 can comprise any one of VH-CDR1 sequences listed in Table 5, 7, 9, or 18, or a variant thereof. In some embodiments, a VH-CDR1 can comprise an amino acid sequence with at least about 100%, 99.9%, 99.8%, 99.7%, 99.6%, 99.5%, 99.4%, 99.3%, 99.2%, 99.1%, 99%, 98%, 97%, 96%, 95%, 94%, 93%, 92%, 91%, 90%, 85%, 80%, 75%, 70%, 65%, 60%, 55%, 50%, 45%, 40%, 35%, 30%, 25%, or at least about 20% sequence identity or sequence similarity to any one of SEQ ID NOs: 1087-1274, 2215-2343, 2989-3124, or 4668-4825. In some embodiments, a VH-CDR1 can comprise an amino acid sequence with at most about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 95.5%, 96%, 96.5%, 97%, 97.5%, 98%, 98.5%, 99%, or at most about 99.5% sequence identity or sequence similarity to any one of SEQ ID NOs: 1087-1274, 2215-2343, 2989-3124, or 4668-4825. In some embodiments, a VH-CDR1 can comprise an amino acid sequence with about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 95.5%, 96%, 96.5%, 97%, 97.5%, 98%, 98.5%, 99%, 99.5%, 99.6%, 99.7%, 99.8%, 99.9%, or about 100% sequence identity or sequence similarity to any one of SEQ ID NOs: 1087-1274, 2215-2343, 2989-3124, or 4668-4825. In some embodiments, a VH-CDR1 can comprise an amino acid sequence of any one of SEQ ID NOs: 1087-1274, 2215-2343, 2989-3124, or 4668-4825, or a variant thereof. In some embodiments, an anti-EPOR or an anti-CD131 antibody that binds to EPO receptor subunit, CD131 subunit, or both subunits of a hetero-EPOR can comprise a VH-CDR1 sequence with at least about 100%, 99.9%, 99.8%, 99.7%, 99.6%, 99.5%, 99.4%, 99.3%, 99.2%, 99.1%, 99%, 98%, 97%, 96%, 95%, 94%, 93%, 92%, 91%, 90%, 85%, 80%, 75%, 70%, 65%, 60%, 55%, 50%, 45%, 40%, 35%, 30%, 25%, or at least about 20% sequence identity or sequence similarity to any one of VH-CDR1 sequences listed in Table 5, 7, 9, or 18. In some embodiments, an anti-EPOR or an anti-CD131 antibody that binds to EPO receptor subunit, CD131 subunit, or both subunits of a hetero-EPOR can comprise a VH-CDR1 sequence at most about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 95.5%, 96%, 96.5%, 97%, 97.5%, 98%, 98.5%, 99%, or at most about 99.5% sequence identity or sequence similarity to any one of VH-CDR1 sequences listed in Table 5, 7, 9, or 18. In some embodiments, an anti-EPOR or an anti-CD131 antibody that binds to EPO receptor subunit, CD131 subunit, or both subunits of a hetero-EPOR can comprise a VH-CDR1 sequence with about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 95.5%, 96%, 96.5%, 97%, 97.5%, 98%, 98.5%, 99%, 99.5%, 99.6%, 99.7%, 99.8%, 99.9%, or about 100% sequence identity or sequence similarity to any one of VH-CDR1 sequences listed in Table 5, 7, 9, or 18. In some embodiments, an anti-EPOR or an anti-CD131 antibody that binds to EPO receptor subunit, CD131 subunit, or both subunits of a hetero-EPOR can comprise any one of VH-CDR1 sequences listed in Table 5, 7, 9, or 18, or a variant thereof. In some embodiments, a variant can comprise a conservatively modified or substituted amino acid sequence variant.

In some embodiments, a VH-CDR2 can comprise an amino acid sequence with at least about 100%, 99.9%, 99.8%, 99.7%, 99.6%, 99.5%, 99.4%, 99.3%, 99.2%, 99.1%, 99%, 98%, 97%, 96%, 95%, 94%, 93%, 92%, 91%, 90%, 85%, 80%, 75%, 70%, 65%, 60%, 55%, 50%, 45%, 40%, 35%, 30%, 25%, or at least about 20% sequence identity or sequence similarity to any one of VH-CDR2 sequences listed in Table 5, 7, 9, or 18. In some embodiments, a VH-CDR2 can comprise an amino acid sequence with at most about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 95.5%, 96%, 96.5%, 97%, 97.5%, 98%, 98.5%, 99%, or at most about 99.5% sequence identity or sequence similarity to any one of VH-CDR2 sequences listed in Table 5, 7, 9, or 18. In some embodiments, a VH-CDR2 can comprise an amino acid sequence with about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 95.5%, 96%, 96.5%, 97%, 97.5%, 98%, 98.5%, 99%, 99.5%, 99.6%, 99.7%, 99.8%, 99.9%, or about 100% sequence identity or sequence similarity to any one of VH-CDR2 sequences listed in Table 5, 7, 9, or 18. In some embodiments, a VH-CDR2 can comprise any one of VH-CDR2 sequences listed in Table 5, 7, 9, or 18, or variant thereof. In some embodiments, a VH-CDR2 can comprise an amino acid sequence with at least about 100%, 99.9%, 99.8%, 99.7%, 99.6%, 99.5%, 99.4%, 99.3%, 99.2%, 99.1%, 99%, 98%, 97%, 96%, 95%, 94%, 93%, 92%, 91%, 90%, 85%, 80%, 75%, 70%, 65%, 60%, 55%, 50%, 45%, 40%, 35%, 30%, 25%, or at least about 20% sequence identity or sequence similarity to any one of SEQ ID NOs: 1275-1462, 2344-2472, 3125-3260, or 4826-4983. In some embodiments, a VH-CDR2 can comprise an amino acid sequence with at most about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 95.5%, 96%, 96.5%, 97%, 97.5%, 98%, 98.5%, 99%, or at most about 99.5% sequence identity or sequence similarity to any one of SEQ ID NOs: 1275-1462, 2344-2472, 3125-3260, or 4826-4983. In some embodiments, a VH-CDR2 can comprise an amino acid sequence with about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 95.5%, 96%, 96.5%, 97%, 97.5%, 98%, 98.5%, 99%, 99.5%, 99.6%, 99.7%, 99.8%, 99.9%, or about 100% sequence identity or sequence similarity to any one of SEQ ID NOs: 1275-1462, 2344-2472, 3125-3260, or 4826-4983. In some embodiments, a VH-CDR2 can comprise an amino acid sequence of any one of SEQ ID NOs: 1275-1462, 2344-2472, 3125-3260, or 4826-4983, or a variant thereof. In some embodiments, an anti-EPOR or an anti-CD131 antibody that binds to EPO receptor subunit, CD131 subunit, or both subunits of a hetero-EPOR can comprise a VH-CDR2 sequence with at least about 100%, 99.9%, 99.8%, 99.7%, 99.6%, 99.5%, 99.4%, 99.3%, 99.2%, 99.1%, 99%, 98%, 97%, 96%, 95%, 94%, 93%, 92%, 91%, 90%, 85%, 80%, 75%, 70%, 65%, 60%, 55%, 50%, 45%, 40%, 35%, 30%, 25%, or at least about 20% sequence identity or sequence similarity to any one of VH-CDR2 sequences listed in Table 5, 7, 9, or 18. In some embodiments, an anti-EPOR or an anti-CD131 antibody that binds to EPO receptor subunit, CD131 subunit, or both subunits of a hetero-EPOR can comprise a VH-CDR2 sequence with at most about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 95.5%, 96%, 96.5%, 97%, 97.5%, 98%, 98.5%, 99%, or at most about 99.5% sequence identity or sequence similarity to any one of VH-CDR2 sequences listed in Table 5, 7, 9, or 18. In some embodiments, an anti-EPOR or an anti-CD131 antibody that binds to EPO receptor subunit, CD131 subunit, or both subunits of a hetero-EPOR can comprise a VH-CDR2 sequence with about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 95.5%, 96%, 96.5%, 97%, 97.5%, 98%, 98.5%, 99%, 99.5%, 99.6%, 99.7%, 99.8%, 99.9%, or about 100% sequence identity or sequence similarity to any one of VH-CDR2 sequences listed in Table 5, 7, 9, or 18. In some embodiments, an anti-EPOR or an anti-CD131 antibody that binds to EPO receptor subunit, CD131 subunit, or both subunits of a hetero-EPOR can comprise any one of VH-CDR2 sequences listed in Table 5, 7, 9, or 18, or a variant thereof. In some embodiments, a variant can comprise a conservatively modified or substituted amino acid sequence variant.

In some embodiments, a VH-CDR3 can comprise an amino acid sequence with at least about 100%, 99.9%, 99.8%, 99.7%, 99.6%, 99.5%, 99.4%, 99.3%, 99.2%, 99.1%, 99%, 98%, 97%, 96%, 95%, 94%, 93%, 92%, 91%, 90%, 85%, 80%, 75%, 70%, 65%, 60%, 55%, 50%, 45%, 40%, 35%, 30%, 25%, or at least about 20% sequence identity or sequence similarity to any one of VH-CDR3 sequences listed in Table 5, 7, 9, 14, 16, or 18. In some embodiments, a VH-CDR3 can comprise an amino acid sequence with at most about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 95.5%, 96%, 96.5%, 97%, 97.5%, 98%, 98.5%, 99%, or at most about 99.5% sequence identity or sequence similarity to any one of VH-CDR3 sequences listed in Table 5, 7, 9, 14, 16, or 18. In some embodiments, a VH-CDR3 can comprise an amino acid sequence with about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 95.5%, 96%, 96.5%, 97%, 97.5%, 98%, 98.5%, 99%, 99.5%, 99.6%, 99.7%, 99.8%, 99.9%, or about 100% sequence identity or sequence similarity to any one of VH-CDR3 sequences listed in Table 5, 7, 9, 14, 16, or 18. In some embodiments, a VH-CDR3 can comprise any one of VH-CDR3 sequences listed in Table 5, 7, 9, 14, 16, or 18, or a variant thereof. In some embodiments, a VH-CDR3 can comprise an amino acid sequence with at least about 100%, 99.9%, 99.8%, 99.7%, 99.6%, 99.5%, 99.4%, 99.3%, 99.2%, 99.1%, 99%, 98%, 97%, 96%, 95%, 94%, 93%, 92%, 91%, 90%, 85%, 80%, 75%, 70%, 65%, 60%, 55%, 50%, 45%, 40%, 35%, 30%, 25%, or at least about 20% sequence identity or sequence similarity to any one of SEQ ID NOs: 1463-1650, 2473-2601, 3261-3396, 4404-4501, 4600-4630, or 4984-5141. In some embodiments, a VH-CDR3 can comprise an amino acid sequence with at most about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 95.5%, 96%, 96.5%, 97%, 97.5%, 98%, 98.5%, 99%, or at most about 99.5% sequence identity or sequence similarity to any one of SEQ ID NOs: 1463-1650, 2473-2601, 3261-3396, 4404-4501, 4600-4630, or 4984-5141. In some embodiments, a VH-CDR3 can comprise an amino acid sequence with about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 95.5%, 96%, 96.5%, 97%, 97.5%, 98%, 98.5%, 99%, 99.5%, 99.6%, 99.7%, 99.8%, 99.9%, or about 100% sequence identity or sequence similarity to any one of SEQ ID NOs: 1463-1650, 2473-2601, 3261-3396, 4404-4501, 4600-4630, or 4984-5141. In some embodiments, a VH-CDR3 can comprise an amino acid sequence of any one of SEQ ID NOs: 1463-1650, 2473-2601, 3261-3396, 4404-4501, 4600-4630, or 4984-5141, or a variant thereof. In some embodiments, an anti-EPOR or an anti-CD131 antibody that binds to EPO receptor subunit, CD131 subunit, or both subunits of a hetero-EPOR can comprise a VH-CDR3 sequence with at least about 100%, 99.9%, 99.8%, 99.7%, 99.6%, 99.5%, 99.4%, 99.3%, 99.2%, 99.1%, 99%, 98%, 97%, 96%, 95%, 94%, 93%, 92%, 91%, 90%, 85%, 80%, 75%, 70%, 65%, 60%, 55%, 50%, 45%, 40%, 35%, 30%, 25%, or at least about 20% sequence identity or sequence similarity to any one of VH-CDR3 sequences listed in Table 5, 7, 9, 14, 16, or 18. In some embodiments, an anti-EPOR or an anti-CD131 antibody that binds to EPO receptor subunit, CD131 subunit, or both subunits of a hetero-EPOR can comprise a VH-CDR3 sequence with at most about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 95.5%, 96%, 96.5%, 97%, 97.5%, 98%, 98.5%, 99%, or at most about 99.5% sequence identity or sequence similarity to any one of VH-CDR3 sequences listed in Table 5, 7, 9, 14, 16, or 18. In some embodiments, an anti-EPOR or an anti-CD131 antibody that binds to EPO receptor subunit, CD131 subunit, or both subunits of a hetero-EPOR can comprise a VH-CDR3 sequence with about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 95.5%, 96%, 96.5%, 97%, 97.5%, 98%, 98.5%, 99%, 99.5%, 99.6%, 99.7%, 99.8%, 99.9%, or about 100% sequence identity or sequence similarity to any one of VH-CDR3 sequences listed in Table 5, 7, 9, 14, 16, or 18. In some embodiments, an anti-EPOR or an anti-CD131 antibody that binds to EPO receptor subunit, CD131 subunit, or both subunits of a hetero-EPOR can comprise any one of VH-CDR3 sequences listed in Table 5, 7, 9, 14, 16, or 18, or a variant thereof. In some embodiments, a variant can comprise a conservatively modified or substituted amino acid sequence variant.

In some embodiments, an anti-EPOR, anti-CD131, or anti-EPO antibody can comprise a VH-CDR1 sequence with at least about 100%, 99.9%, 99.8%, 99.7%, 99.6%, 99.5%, 99.4%, 99.3%, 99.2%, 99.1%, 99%, 98%, 97%, 96%, 95%, 94%, 93%, 92%, 91%, 90%, 85%, 80%, 75%, 70%, 65%, 60%, 55%, 50%, 45%, 40%, 35%, 30%, 25%, or at least about 20% sequence identity or sequence similarity to any one of VH-CDR1 sequences listed in Table 5, 7, 9, or 18, a VH-CDR2 sequence with at least about 100%, 99.9%, 99.8%, 99.7%, 99.6%, 99.5%, 99.4%, 99.3%, 99.2%, 99.1%, 99%, 98%, 97%, 96%, 95%, 94%, 93%, 92%, 91%, 90%, 85%, 80%, 75%, 70%, 65%, 60%, 55%, 50%, 45%, 40%, 35%, 30%, 25%, or at least about 20% sequence identity or sequence similarity to any one of VH-CDR2 sequences listed in Table 5, 7, 9, or 18, and a VH-CDR3 sequence with at least about 100%, 99.9%, 99.8%, 99.7%, 99.6%, 99.5%, 99.4%, 99.3%, 99.2%, 99.1%, 99%, 98%, 97%, 96%, 95%, 94%, 93%, 92%, 91%, 90%, 85%, 80%, 75%, 70%, 65%, 60%, 55%, 50%, 45%, 40%, 35%, 30%, 25%, or at least about 20% sequence identity or sequence similarity to any one of VH-CDR3 sequences listed in Table 5, 7, 9, 14, 16, or 18. In some embodiments, an anti-EPOR, anti-CD131, or anti-EPO antibody can comprise any one of VH-CDR1 sequences listed in Table 5, 7, 9, or 18, or a variant thereof, VH-CDR2 sequences listed in Table 5, 7, 9, or 18, or a variant thereof, and VH-CDR3 sequences listed in Table 5, 7, 9, 14, 16, or 18, or a variant thereof. In some embodiments, a variant can comprise a conservatively modified or substituted amino acid sequence variant.

In some embodiments, an anti-EPOR, anti-CD131, or anti-EPO antibody can comprise a VH-CDR1 comprising an amino acid sequence with at least about 100%, 99.9%, 99.8%, 99.7%, 99.6%, 99.5%, 99.4%, 99.3%, 99.2%, 99.1%, 99%, 98%, 97%, 96%, 95%, 94%, 93%, 92%, 91%, 90%, 85%, 80%, 75%, 70%, 65%, 60%, 55%, 50%, 45%, 40%, 35%, 30%, 25%, or at least about 20% sequence identity or sequence similarity to any one of SEQ ID NOs: 1087-1274, 2215-2343, 2989-3124, or 4668-4825, a VH-CDR2 comprising an amino acid sequence with at least about 100%, 99.9%, 99.8%, 99.7%, 99.6%, 99.5%, 99.4%, 99.3%, 99.2%, 99.1%, 99%, 98%, 97%, 96%, 95%, 94%, 93%, 92%, 91%, 90%, 85%, 80%, 75%, 70%, 65%, 60%, 55%, 50%, 45%, 40%, 35%, 30%, 25%, or at least about 20% sequence identity or sequence similarity to any one of SEQ ID NOs: 1275-1462, 2344-2472, 3125-3260, or 4826-4983, and a VH-CDR3 comprising an amino acid sequence with at least about 100%, 99.9%, 99.8%, 99.7%, 99.6%, 99.5%, 99.4%, 99.3%, 99.2%, 99.1%, 99%, 98%, 97%, 96%, 95%, 94%, 93%, 92%, 91%, 90%, 85%, 80%, 75%, 70%, 65%, 60%, 55%, 50%, 45%, 40%, 35%, 30%, 25%, or at least about 20% sequence identity or sequence similarity to any one of SEQ ID NOs: 1463-1650, 2473-2601, 3261-3396, 4404-4501, 4600-4630, or 4984-5141.

In some embodiments, an anti-EPOR, anti-CD131, or anti-EPO antibody can comprise a VH-CDR1 comprising an amino acid sequence with at most about 100%, 99.9%, 99.8%, 99.7%, 99.6%, 99.5%, 99.4%, 99.3%, 99.2%, 99.1%, 99%, 98%, 97%, 96%, 95%, 94%, 93%, 92%, 91%, 90%, 85%, 80%, 75%, 70%, 65%, 60%, 55%, 50%, 45%, 40%, 35%, 30%, 25%, or at most about 20% sequence identity or sequence similarity to any one of SEQ ID NOs: 1087-1274, 2215-2343, 2989-3124, or 4668-4825, a VH-CDR2 comprising an amino acid sequence with at most about 100%, 99.9%, 99.8%, 99.7%, 99.6%, 99.5%, 99.4%, 99.3%, 99.2%, 99.1%, 99%, 98%, 97%, 96%, 95%, 94%, 93%, 92%, 91%, 90%, 85%, 80%, 75%, 70%, 65%, 60%, 55%, 50%, 45%, 40%, 35%, 30%, 25%, or at most about 20% sequence identity or sequence similarity to any one of SEQ ID NOs: 1275-1462, 2344-2472, 3125-3260, or 4826-4983, and a VH-CDR3 comprising an amino acid sequence with at most about 100%, 99.9%, 99.8%, 99.7%, 99.6%, 99.5%, 99.4%, 99.3%, 99.2%, 99.1%, 99%, 98%, 97%, 96%, 95%, 94%, 93%, 92%, 91%, 90%, 85%, 80%, 75%, 70%, 65%, 60%, 55%, 50%, 45%, 40%, 35%, 30%, 25%, or at most about 20% sequence identity or sequence similarity to any one of SEQ ID NOs: 1463-1650, 2473-2601, 3261-3396, 4404-4501, 4600-4630, or 4984-5141.

In some embodiments, an anti-EPOR, anti-CD131, or anti-EPO antibody can comprise a VH-CDR1 comprising an amino acid sequence with about 100%, 99.9%, 99.8%, 99.7%, 99.6%, 99.5%, 99.4%, 99.3%, 99.2%, 99.1%, 99%, 98%, 97%, 96%, 95%, 94%, 93%, 92%, 91%, 90%, 85%, 80%, 75%, 70%, 65%, 60%, 55%, 50%, 45%, 40%, 35%, 30%, 25%, or about 20% sequence identity or sequence similarity to any one of SEQ ID NOs: 1087-1274, 2215-2343, 2989-3124, or 4668-4825, a VH-CDR2 comprising an amino acid sequence with about 100%, 99.9%, 99.8%, 99.7%, 99.6%, 99.5%, 99.4%, 99.3%, 99.2%, 99.1%, 99%, 98%, 97%, 96%, 95%, 94%, 93%, 92%, 91%, 90%, 85%, 80%, 75%, 70%, 65%, 60%, 55%, 50%, 45%, 40%, 35%, 30%, 25%, or about 20% sequence identity or sequence similarity to any one of SEQ ID NOs: 1275-1462, 2344-2472, 3125-3260, or 4826-4983, and a VH-CDR3 comprising an amino acid sequence with about 100%, 99.9%, 99.8%, 99.7%, 99.6%, 99.5%, 99.4%, 99.3%, 99.2%, 99.1%, 99%, 98%, 97%, 96%, 95%, 94%, 93%, 92%, 91%, 90%, 85%, 80%, 75%, 70%, 65%, 60%, 55%, 50%, 45%, 40%, 35%, 30%, 25%, or about 20% sequence identity or sequence similarity to any one of SEQ ID NOs: 1463-1650, 2473-2601, 3261-3396, 4404-4501, 4600-4630, or 4984-5141.

In some embodiments, an anti-EPOR, anti-CD131, or anti-EPO antibody can comprise a VH-CDR1 comprising an amino acid sequence of any one of SEQ ID NOs: 1087-1274, 2215-2343, 2989-3124, or 4668-4825, or a variant thereof, a VH-CDR2 comprising an amino acid sequence of any one of SEQ ID NOs: 1275-1462, 2344-2472, 3125-3260, or 4826-4983, or a variant thereof, and a VH-CDR3 comprising an amino acid sequence of any one of SEQ ID NOs: 1463-1650, 2473-2601, 3261-3396, 4404-4501, 4600-4630, or 4984-5141, or a variant thereof. In some embodiments, a variant can comprise a conservatively modified or substituted amino acid sequence variant.

In some embodiments, a VL can comprise a VL complementarity determining region 1 (VL-CDR1), a VL-CDR2, or a VL-CDR3. In some embodiments, a VL can comprise a VL complementarity determining region 1 (VL-CDR1), a VL-CDR2, and a VL-CDR3. In some embodiments, VL-CDR1, VL-CDR2, and/or VL-CDR3 can be defined according to Kabat, Chothia, IMGT, AbM, or Contact.

In some embodiments, a VL-CDR1 can comprise an amino acid sequence with at least about 100%, 99.9%, 99.8%, 99.7%, 99.6%, 99.5%, 99.4%, 99.3%, 99.2%, 99.1%, 99%, 98%, 97%, 96%, 95%, 94%, 93%, 92%, 91%, 90%, 85%, 80%, 75%, 70%, 65%, 60%, 55%, 50%, 45%, 40%, 35%, 30%, 25%, or at least about 20% sequence identity or sequence similarity to any one of VL-CDR1 sequences listed in Table 5, 7, 9, or 19. In some embodiments, a VL-CDR1 can comprise an amino acid sequence with at most about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 95.5%, 96%, 96.5%, 97%, 97.5%, 98%, 98.5%, 99%, or at most about 99.5% sequence identity or sequence similarity to any one of VL-CDR1 sequences listed in Table 5, 7, 9, or 19. In some embodiments, a VL-CDR1 can comprise an amino acid sequence with about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 95.5%, 96%, 96.5%, 97%, 97.5%, 98%, 98.5%, 99%, 99.5%, 99.6%, 99.7%, 99.8%, 99.9%, or about 100% sequence identity or sequence similarity to any one of VL-CDR1 sequences listed in Table 5, 7, 9, or 19. In some embodiments, a VL-CDR1 can comprise any one of VL-CDR1 sequences listed in Table 5, 7, 9, or 19, or a variant thereof. In some embodiments, a VL-CDR1 can comprise an amino acid sequence with at least about 100%, 99.9%, 99.8%, 99.7%, 99.6%, 99.5%, 99.4%, 99.3%, 99.2%, 99.1%, 99%, 98%, 97%, 96%, 95%, 94%, 93%, 92%, 91%, 90%, 85%, 80%, 75%, 70%, 65%, 60%, 55%, 50%, 45%, 40%, 35%, 30%, 25%, or at least about 20% sequence identity or sequence similarity to any one of SEQ ID NOs: 1651-1838, 2602-2730, 3397-3532, or 5142-5299. In some embodiments, a VL-CDR1 can comprise an amino acid sequence with at most about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 95.5%, 96%, 96.5%, 97%, 97.5%, 98%, 98.5%, 99%, or at most about 99.5% sequence identity or sequence similarity to any one of SEQ ID NOs: 1651-1838, 2602-2730, 3397-3532, or 5142-5299. In some embodiments, a VL-CDR1 can comprise an amino acid sequence with about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 95.5%, 96%, 96.5%, 97%, 97.5%, 98%, 98.5%, 99%, 99.5%, 99.6%, 99.7%, 99.8%, 99.9%, or about 100% sequence identity or sequence similarity to any one of SEQ ID NOs: 1651-1838, 2602-2730, 3397-3532, or 5142-5299. In some embodiments, a VL-CDR1 can comprise an amino acid sequence of any one of SEQ ID NOs: 1651-1838, 2602-2730, 3397-3532, or 5142-5299, or a variant thereof. In some embodiments, an anti-EPOR or an anti-CD131 antibody that binds to EPO receptor subunit, CD131 subunit, or both subunits of a hetero-EPOR can comprise a VL-CDR1 sequence with at least about 100%, 99.9%, 99.8%, 99.7%, 99.6%, 99.5%, 99.4%, 99.3%, 99.2%, 99.1%, 99%, 98%, 97%, 96%, 95%, 94%, 93%, 92%, 91%, 90%, 85%, 80%, 75%, 70%, 65%, 60%, 55%, 50%, 45%, 40%, 35%, 30%, 25%, or at least about 20% sequence identity or sequence similarity to any one of VL-CDR1 sequences listed in Table 5, 7, 9, or 19. In some embodiments, an anti-EPOR or an anti-CD131 antibody that binds to EPO receptor subunit, CD131 subunit, or both subunits of a hetero-EPOR can comprise a VL-CDR1 sequence with at most about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 95.5%, 96%, 96.5%, 97%, 97.5%, 98%, 98.5%, 99%, or at most about 99.5% sequence identity or sequence similarity to any one of VL-CDR1 sequences listed in Table 5, 7, 9, or 19. In some embodiments, an anti-EPOR or an anti-CD131 antibody that binds to EPO receptor subunit, CD131 subunit, or both subunits of a hetero-EPOR can comprise a VL-CDR1 sequence with about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 95.5%, 96%, 96.5%, 97%, 97.5%, 98%, 98.5%, 99%, 99.5%, 99.6%, 99.7%, 99.8%, 99.9%, or about 100% sequence identity or sequence similarity to any one of VL-CDR1 sequences listed in Table 5, 7, 9, or 19. In some embodiments an anti-EPOR or an anti-CD131 antibody can comprise any one of VL-CDR1 sequences listed in Table 5, 7, 9, or 19, or a variant thereof. In some embodiments, a variant can comprise a conservatively modified or substituted amino acid sequence variant.

In some embodiments, a VL-CDR2 can comprise an amino acid sequence with at least about 100%, 99.9%, 99.8%, 99.7%, 99.6%, 99.5%, 99.4%, 99.3%, 99.2%, 99.1%, 99%, 98%, 97%, 96%, 95%, 94%, 93%, 92%, 91%, 90%, 85%, 80%, 75%, 70%, 65%, 60%, 55%, 50%, 45%, 40%, 35%, 30%, 25%, or at least about 20% sequence identity or sequence similarity to any one of VL-CDR2 sequences listed in Table 5, 7, 9, or 19. In some embodiments, a VL-CDR2 can comprise an amino acid sequence with at most about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 95.5%, 96%, 96.5%, 97%, 97.5%, 98%, 98.5%, 99%, or at most about 99.5% sequence identity or sequence similarity to any one of VL-CDR2 sequences listed in Table 5, 7, 9, or 19. In some embodiments, a VL-CDR2 can comprise an amino acid sequence with about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 95.5%, 96%, 96.5%, 97%, 97.5%, 98%, 98.5%, 99%, 99.5%, 99.6%, 99.7%, 99.8%, 99.9%, or about 100% sequence identity or sequence similarity to any one of VL-CDR2 sequences listed in Table 5, 7, 9, or 19. In some embodiments, a VL-CDR2 can comprise any one of VL-CDR2 sequences listed in Table 5, 7, 9, or 19, or a variant thereof. In some embodiments, a VL-CDR2 can comprise an amino acid sequence with at least about 100%, 99.9%, 99.8%, 99.7%, 99.6%, 99.5%, 99.4%, 99.3%, 99.2%, 99.1%, 99%, 98%, 97%, 96%, 95%, 94%, 93%, 92%, 91%, 90%, 85%, 80%, 75%, 70%, 65%, 60%, 55%, 50%, 45%, 40%, 35%, 30%, 25%, or at least about 20% sequence identity or sequence similarity to any one of SEQ ID NOs: 1839-2026, 2731-2859, 3533-3668, or 5300-5457. In some embodiments, a VL-CDR2 can comprise an amino acid sequence with at most about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 95.5%, 96%, 96.5%, 97%, 97.5%, 98%, 98.5%, 99%, or at most about 99.5% sequence identity or sequence similarity to any one of SEQ ID NOs: 1839-2026, 2731-2859, 3533-3668, or 5300-5457. In some embodiments, a VL-CDR2 can comprise an amino acid sequence with about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 95.5%, 96%, 96.5%, 97%, 97.5%, 98%, 98.5%, 99%, 99.5%, 99.6%, 99.7%, 99.8%, 99.9%, or about 100% sequence identity or sequence similarity to any one of SEQ ID NOs: 1839-2026, 2731-2859, 3533-3668, or 5300-5457. In some embodiments, a VL-CDR2 can comprise an amino acid sequence of any one of SEQ ID NOs: 1839-2026, 2731-2859, 3533-3668, or 5300-5457, or a variant thereof. In some embodiments, an anti-EPOR or an anti-CD131 antibody that binds to EPO receptor subunit, CD131 subunit, or both subunits of a hetero-EPOR can comprise a VL-CDR2 sequence with at least about 100%, 99.9%, 99.8%, 99.7%, 99.6%, 99.5%, 99.4%, 99.3%, 99.2%, 99.1%, 99%, 98%, 97%, 96%, 95%, 94%, 93%, 92%, 91%, 90%, 85%, 80%, 75%, 70%, 65%, 60%, 55%, 50%, 45%, 40%, 35%, 30%, 25%, or at least about 20% sequence identity or sequence similarity to any one of VL-CDR2 sequences listed in Table 5, 7, 9, or 19. In some embodiments, an anti-EPOR or an anti-CD131 antibody that binds to EPO receptor subunit, CD131 subunit, or both subunits of a hetero-EPOR can comprise a VL-CDR2 sequence with at most about 70%, 89%, 90%, 91%, 92%, 93%, 94%, 95%, 95.5%, 96%, 96.5%, 97%, 97.5%, 98%, 98.5%, 99%, or at most about 99.5% sequence identity or sequence similarity to any one of VL-CDR2 sequences listed in Table 5, 7, 9, or 19. In some embodiments, an anti-EPOR or an anti-CD131 antibody that binds to EPO receptor subunit, CD131 subunit, or both subunits of a hetero-EPOR can comprise a VL-CDR2 sequence with about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 95.5%, 96%, 96.5%, 97%, 97.5%, 98%, 98.5%, 99%, 99.5%, 99.6%, 99.7%, 99.8%, 99.9%, or about 100% sequence identity or sequence similarity to any one of VL-CDR2 sequences listed in Table 5, 7, 9, or 19. In some embodiments, an anti-EPOR or an anti-CD131 antibody that binds to EPO receptor subunit, CD131 subunit, or both subunits of a hetero-EPOR can comprise any one of VL-CDR2 sequences listed in Table 5, 7, 9, or 19, or a variant thereof. In some embodiments, a variant can comprise a conservatively modified or substituted amino acid sequence variant.

In some embodiments, a VL-CDR3 can comprise an amino acid sequence with at least about 100%, 99.9%, 99.8%, 99.7%, 99.6%, 99.5%, 99.4%, 99.3%, 99.2%, 99.1%, 99%, 98%, 97%, 96%, 95%, 94%, 93%, 92%, 91%, 90%, 85%, 80%, 75%, 70%, 65%, 60%, 55%, 50%, 45%, 40%, 35%, 30%, 25%, or at least about 20% sequence identity or sequence similarity to any one of VL-CDR3 sequences listed in Table 5, 7, 9, 14, 16, or 19. In some embodiments, a VL-CDR3 can comprise an amino acid sequence with at most about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 95.5%, 96%, 96.5%, 97%, 97.5%, 98%, 98.5%, 99%, or at most about 99.5% sequence identity or sequence similarity to any one of VL-CDR3 sequences listed in Table 5, 7, 9, 14, 16, or 19. In some embodiments, a VL-CDR3 can comprise an amino acid sequence with about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 95.5%, 96%, 96.5%, 97%, 97.5%, 98%, 98.5%, 99%, 99.5%, 99.6%, 99.7%, 99.8%, 99.9%, or about 100% sequence identity or sequence similarity to any one of VL-CDR3 sequences listed in Table 5, 7, 9, 14, 16, or 19. In some embodiments, a VL-CDR3 can comprise any one of VL-CDR3 sequences listed in Table 5, 7, 9, 14, 16, or 19, or a variant thereof. In some embodiments, a VL-CDR3 can comprise an amino acid sequence with at least about 100%, 99.9%, 99.8%, 99.7%, 99.6%, 99.5%, 99.4%, 99.3%, 99.2%, 99.1%, 99%, 98%, 97%, 96%, 95%, 94%, 93%, 92%, 91%, 90%, 85%, 80%, 75%, 70%, 65%, 60%, 55%, 50%, 45%, 40%, 35%, 30%, 25%, or at least about 20% sequence identity or sequence similarity to any one of SEQ ID NOs: 2027-2214, 2680-2988, 3669-3804, 4502-4599, 4631-4667, or 5458-5615. In some embodiments, a VL-CDR3 can comprise an amino acid sequence with at most about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 95.5%, 96%, 96.5%, 97%, 97.5%, 98%, 98.5%, 99%, or at most about 99.5% sequence identity or sequence similarity to any one of SEQ ID NOs: 2027-2214, 2680-2988, 3669-3804, 4502-4599, 4631-4667, or 5458-5615. In some embodiments, a VL-CDR3 can comprise an amino acid sequence with about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 95.5%, 96%, 96.5%, 97%, 97.5%, 98%, 98.5%, 99%, 99.5%, 99.6%, 99.7%, 99.8%, 99.9%, or about 100% sequence identity or sequence similarity to any one of SEQ ID NOs: 2027-2214, 2680-2988, 3669-3804, 4502-4599, 4631-4667, or 5458-5615. In some embodiments, a VL-CDR3 can comprise an amino acid sequence of any one of SEQ ID NOs: 2027-2214, 2680-2988, 3669-3804, 4502-4599, 4631-4667, or 5458-5615, or a variant thereof. In some embodiments, an anti-EPOR or an anti-CD131 antibody that binds to EPO receptor subunit, CD131 subunit, or both subunits of a hetero-EPOR can comprise a VL-CDR3 sequence with at least about 100%, 99.9%, 99.8%, 99.7%, 99.6%, 99.5%, 99.4%, 99.3%, 99.2%, 99.1%, 99%, 98%, 97%, 96%, 95%, 94%, 93%, 92%, 91%, 90%, 85%, 80%, 75%, 70%, 65%, 60%, 55%, 50%, 45%, 40%, 35%, 30%, 25%, or at least about 20% sequence identity or sequence similarity to any one of VL-CDR3 sequences listed in Table 5, 7, 9, 14, 16, or 19. In some embodiments, an anti-EPOR or an anti-CD131 antibody that binds to EPO receptor subunit, CD131 subunit, or both subunits of a hetero-EPOR can comprise a VL-CDR3 sequence with at most about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 95.5%, 96%, 96.5%, 97%, 97.5%, 98%, 98.5%, 99%, or at most about 99.5% sequence identity or sequence similarity to any one of VL-CDR3 sequences listed in Table 5, 7, 9, 14, 16, or 19. In some embodiments, an anti-EPOR or an anti-CD131 antibody that binds to EPO receptor subunit, CD131 subunit, or both subunits of a hetero-EPOR can comprise a VL-CDR3 sequence with about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 95.5%, 96%, 96.5%, 97%, 97.5%, 98%, 98.5%, 99%, 99.5%, 99.6%, 99.7%, 99.8%, 99.9%, or about 100% sequence identity or sequence similarity to any one of VL-CDR3 sequences listed in Table 5, 7, 9, 14, 16, or 19. In some embodiments an anti-EPOR or an anti-CD131 antibody that binds to EPO receptor subunit, CD131 subunit, or both subunits of a hetero-EPOR can comprise any one of VL-CDR3 sequences listed in Table 5, 7, 9, 14, 16, or 19, or a variant thereof. In some embodiments, a variant can comprise a conservatively modified or substituted amino acid sequence variant.

In some embodiments, an anti-EPOR, anti-CD131, or anti-EPO antibody can comprise a VL-CDR1 sequence with at least about 100%, 99.9%, 99.8%, 99.7%, 99.6%, 99.5%, 99.4%, 99.3%, 99.2%, 99.1%, 99%, 98%, 97%, 96%, 95%, 94%, 93%, 92%, 91%, 90%, 85%, 80%, 75%, 70%, 65%, 60%, 55%, 50%, 45%, 40%, 35%, 30%, 25%, or at least about 20% sequence identity or sequence similarity to any one of VL-CDR1 sequences listed in Table 5, 7, 9, or 19, a VL-CDR2 sequence with at least about 100%, 99.9%, 99.8%, 99.7%, 99.6%, 99.5%, 99.4%, 99.3%, 99.2%, 99.1%, 99%, 98%, 97%, 96%, 95%, 94%, 93%, 92%, 91%, 90%, 85%, 80%, 75%, 70%, 65%, 60%, 55%, 50%, 45%, 40%, 35%, 30%, 25%, or at least about 20% sequence identity or sequence similarity to any one of VL-CDR2 sequences listed in Table 5, 7, 9, or 19, and a VL-CDR3 sequence with at least about 100%, 99.9%, 99.8%, 99.7%, 99.6%, 99.5%, 99.4%, 99.3%, 99.2%, 99.1%, 99%, 98%, 97%, 96%, 95%, 94%, 93%, 92%, 91%, 90%, 85%, 80%, 75%, 70%, 65%, 60%, 55%, 50%, 45%, 40%, 35%, 30%, 25%, or at least about 20% sequence identity or sequence similarity to any one of VL-CDR3 sequences listed in Table 5, 7, 9, 14, 16, or 19. In some embodiments an anti-EPOR antibody or an anti-CD131 antibody can comprise any one of VL-CDR1 sequences listed in Table 5, 7, 9, or 19, or a variant thereof, VL-CDR2 sequences listed in Table 5, 7, 9, or 19, or a variant thereof, and VL-CDR3 sequences listed in Table 5, 7, 9, 14, 16, or 19, or a variant thereof. In some embodiments, a variant can comprise a conservatively modified or substituted amino acid sequence variant.

In some embodiments, an anti-EPOR, anti-CD131, or anti-EPO antibody can comprise a VL-CDR1 comprising an amino acid sequence with at least about 100%, 99.9%, 99.8%, 99.7%, 99.6%, 99.5%, 99.4%, 99.3%, 99.2%, 99.1%, 99%, 98%, 97%, 96%, 95%, 94%, 93%, 92%, 91%, 90%, 85%, 80%, 75%, 70%, 65%, 60%, 55%, 50%, 45%, 40%, 35%, 30%, 25%, or at least about 20% sequence identity or sequence similarity to any one of SEQ ID NOs: 1651-1838, 2602-2730, 3397-3532, or 5142-5299, a VL-CDR2 comprising an amino acid sequence with at least about 100%, 99.9%, 99.8%, 99.7%, 99.6%, 99.5%, 99.4%, 99.3%, 99.2%, 99.1%, 99%, 98%, 97%, 96%, 95%, 94%, 93%, 92%, 91%, 90%, 85%, 80%, 75%, 70%, 65%, 60%, 55%, 50%, 45%, 40%, 35%, 30%, 25%, or at least about 20% sequence identity or sequence similarity to any one of SEQ ID NOs: 1839-2026, 2731-2859, 3533-3668, or 5300-5457, and a VL-CDR3 comprising an amino acid sequence with at least about 100%, 99.9%, 99.8%, 99.7%, 99.6%, 99.5%, 99.4%, 99.3%, 99.2%, 99.1%, 99%, 98%, 97%, 96%, 95%, 94%, 93%, 92%, 91%, 90%, 85%, 80%, 75%, 70%, 65%, 60%, 55%, 50%, 45%, 40%, 35%, 30%, 25%, or at least about 20% sequence identity or sequence similarity to any one of SEQ ID NOs: 2027-2214, 2680-2988, 3669-3804, 4502-4599, 4631-4667, or 5458-5615.

In some embodiments, an anti-EPOR, anti-CD131, or anti-EPO antibody can comprise a VL-CDR1 comprising an amino acid sequence with at most about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 95.5%, 96%, 96.5%, 97%, 97.5%, 98%, 98.5%, 99%, or at most about 99.5% sequence identity or sequence similarity to any one of SEQ ID NOs: 1651-1838, 2602-2730, 3397-3532, or 5142-5299, a VL-CDR2 comprising an amino acid sequence with at most about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 95.5%, 96%, 96.5%, 97%, 97.5%, 98%, 98.5%, 99%, or at most about 99.5% sequence identity or sequence similarity to any one of SEQ ID NOs: 1839-2026, 2731-2859, 3533-3668, or 5300-5457, and a VL-CDR3 comprising an amino acid sequence with at most about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 95.5%, 96%, 96.5%, 97%, 97.5%, 98%, 98.5%, 99%, or at most about 99.5% sequence identity or sequence similarity to any one of SEQ ID NOs: 2027-2214, 2680-2988, 3669-3804, 4502-4599, 4631-4667, or 5458-5615.

In some embodiments, an anti-EPOR, anti-CD131, or anti-EPO antibody can comprise a VL-CDR1 comprising an amino acid sequence with about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 95.5%, 96%, 96.5%, 97%, 97.5%, 98%, 98.5%, 99%, 99.5%, 99.6%, 99.7%, 99.8%, 99.9%, or about 100% sequence identity or sequence similarity to any one of SEQ ID NOs: 1651-1838, 2602-2730, 3397-3532, or 5142-5299, a VL-CDR2 comprising an amino acid sequence with about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 95.5%, 96%, 96.5%, 97%, 97.5%, 98%, 98.5%, 99%, 99.5%, 99.6%, 99.7%, 99.8%, 99.9%, or about 100% sequence identity or sequence similarity to any one of SEQ ID NOs: 1839-2026, 2731-2859, 3533-3668, or 5300-5457, and a VL-CDR3 comprising an amino acid sequence with about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 95.5%, 96%, 96.5%, 97%, 97.5%, 98%, 98.5%, 99%, 99.5%, 99.6%, 99.7%, 99.8%, 99.9%, or about 100% sequence identity or sequence similarity to any one of SEQ ID NOs: 2027-2214, 2680-2988, 3669-3804, 4502-4599, 4631-4667, or 5458-5615.

In some embodiments, an anti-EPOR, anti-CD131, or anti-EPO antibody can comprise a VL-CDR1 comprising an amino acid sequence of any one of SEQ ID NOs: 1651-1838, 2602-2730, 3397-3532, or 5142-5299, or a variant thereof, a VL-CDR2 comprising an amino acid sequence of any one of SEQ ID NOs: 1839-2026, 2731-2859, 3533-3668, or 5300-5457, or a variant thereof, and a VL-CDR3 comprising an amino acid sequence of any one of SEQ ID NOs: 2027-2214, 2680-2988, 3669-3804, 4502-4599, 4631-4667, or 5458-5615, or a variant thereof. In some embodiments, a variant can comprise a conservatively modified or substituted amino acid sequence variant.

In some embodiments, an anti-EPOR, anti-CD131, or anti-EPO antibody can comprise a VH-CDR1 sequence with at least about 100%, 99.9%, 99.8%, 99.7%, 99.6%, 99.5%, 99.4%, 99.3%, 99.2%, 99.1%, 99%, 98%, 97%, 96%, 95%, 94%, 93%, 92%, 91%, 90%, 85%, 80%, 75%, 70%, 65%, 60%, 55%, 50%, 45%, 40%, 35%, 30%, 25%, or at least about 20% sequence identity or sequence similarity to any one of VH-CDR1 sequences listed in Table 5, 7, 9, or 18, a VH-CDR2 sequence with at least about 100%, 99.9%, 99.8%, 99.7%, 99.6%, 99.5%, 99.4%, 99.3%, 99.2%, 99.1%, 99%, 98%, 97%, 96%, 95%, 94%, 93%, 92%, 91%, 90%, 85%, 80%, 75%, 70%, 65%, 60%, 55%, 50%, 45%, 40%, 35%, 30%, 25%, or at least about 20% sequence identity or sequence similarity to any one of VH-CDR2 sequences listed in Table 5, 7, 9, or 18, a VH-CDR3 sequence with at least about 100%, 99.9%, 99.8%, 99.7%, 99.6%, 99.5%, 99.4%, 99.3%, 99.2%, 99.1%, 99%, 98%, 97%, 96%, 95%, 94%, 93%, 92%, 91%, 90%, 85%, 80%, 75%, 70%, 65%, 60%, 55%, 50%, 45%, 40%, 35%, 30%, 25%, or at least about 20% sequence identity or sequence similarity to any one of VH-CDR3 sequences listed in Table 5, 7, 9, 14, 16, or 18, a VL-CDR1 sequence with at least about 100%, 99.9%, 99.8%, 99.7%, 99.6%, 99.5%, 99.4%, 99.3%, 99.2%, 99.1%, 99%, 98%, 97%, 96%, 95%, 94%, 93%, 92%, 91%, 90%, 85%, 80%, 75%, 70%, 65%, 60%, 55%, 50%, 45%, 40%, 35%, 30%, 25%, or at least about 20% sequence identity or sequence similarity to any one of VL-CDR1 sequences listed in Table 5, 7, 9, or 19, a VL-CDR2 sequence with at least about 100%, 99.9%, 99.8%, 99.7%, 99.6%, 99.5%, 99.4%, 99.3%, 99.2%, 99.1%, 99%, 98%, 97%, 96%, 95%, 94%, 93%, 92%, 91%, 90%, 85%, 80%, 75%, 70%, 65%, 60%, 55%, 50%, 45%, 40%, 35%, 30%, 25%, or at least about 20% sequence identity or sequence similarity to any one of VL-CDR2 sequences listed in Table 5, 7, 9, or 19, and a VL-CDR3 sequence with at least about 100%, 99.9%, 99.8%, 99.7%, 99.6%, 99.5%, 99.4%, 99.3%, 99.2%, 99.1%, 99%, 98%, 97%, 96%, 95%, 94%, 93%, 92%, 91%, 90%, 85%, 80%, 75%, 70%, 65%, 60%, 55%, 50%, 45%, 40%, 35%, 30%, 25%, or at least about 20% sequence identity or sequence similarity to any one of VL-CDR3 sequences listed in Table 5, 7, 9, 14, 16, or 19. In some embodiments, an anti-EPOR, anti-CD131, or anti-EPO antibody can comprise any one of VH-CDR1 sequences listed in Table 5, 7, 9, or 18, or a variant thereof, VH-CDR2 sequences listed in Table 5, 7, 9, or 18, or a variant thereof, VH-CDR3, VL-CDR1 sequences listed in Table 5, 7, 9, or 19, or a variant thereof, VL-CDR2 sequences listed in Table 5, 7, 9, or 19, or a variant thereof, and VL-CDR3 sequences listed in Table 5, 7, 9, 14, 16, or 19, or a variant thereof. In some embodiments, a variant can comprise a conservatively modified or substituted amino acid sequence variant.

In some embodiments, an anti-EPOR, anti-CD131, or anti-EPO antibody can comprise a VH-CDR1 comprising an amino acid sequence with at least about 100%, 99.9%, 99.8%, 99.7%, 99.6%, 99.5%, 99.4%, 99.3%, 99.2%, 99.1%, 99%, 98%, 97%, 96%, 95%, 94%, 93%, 92%, 91%, 90%, 85%, 80%, 75%, 70%, 65%, 60%, 55%, 50%, 45%, 40%, 35%, 30%, 25%, or at least about 20% sequence identity or sequence similarity to any one of SEQ ID NOs: 1087-1274, 2215-2343, 2989-3124, or 4668-4825, a VH-CDR2 comprising an amino acid sequence with at least about 100%, 99.9%, 99.8%, 99.7%, 99.6%, 99.5%, 99.4%, 99.3%, 99.2%, 99.1%, 99%, 98%, 97%, 96%, 95%, 94%, 93%, 92%, 91%, 90%, 85%, 80%, 75%, 70%, 65%, 60%, 55%, 50%, 45%, 40%, 35%, 30%, 25%, or at least about 20% sequence identity or sequence similarity to any one of SEQ ID NOs: 1275-1462, 2344-2472, 3125-3260, or 4826-4983, a VH-CDR3 comprising an amino acid sequence with at least about 100%, 99.9%, 99.8%, 99.7%, 99.6%, 99.5%, 99.4%, 99.3%, 99.2%, 99.1%, 99%, 98%, 97%, 96%, 95%, 94%, 93%, 92%, 91%, 90%, 85%, 80%, 75%, 70%, 65%, 60%, 55%, 50%, 45%, 40%, 35%, 30%, 25%, or at least about 20% sequence identity or sequence similarity to any one of SEQ ID NOs: 1463-1650, 2473-2601, 3261-3396, 4404-4501, 4600-4630, or 4984-5141, a VL-CDR1 comprising an amino acid sequence with at least about 100%, 99.9%, 99.8%, 99.7%, 99.6%, 99.5%, 99.4%, 99.3%, 99.2%, 99.1%, 99%, 98%, 97%, 96%, 95%, 94%, 93%, 92%, 91%, 90%, 85%, 80%, 75%, 70%, 65%, 60%, 55%, 50%, 45%, 40%, 35%, 30%, 25%, or at least about 20% sequence identity or sequence similarity to any one of SEQ ID NOs: 1651-1838, 2602-2730, 3397-3532, or 5142-5299, a VL-CDR2 comprising an amino acid sequence with at least about 100%, 99.9%, 99.8%, 99.7%, 99.6%, 99.5%, 99.4%, 99.3%, 99.2%, 99.1%, 99%, 98%, 97%, 96%, 95%, 94%, 93%, 92%, 91%, 90%, 85%, 80%, 75%, 70%, 65%, 60%, 55%, 50%, 45%, 40%, 35%, 30%, 25%, or at least about 20% sequence identity or sequence similarity to any one of SEQ ID NOs: 1839-2026, 2731-2859, 3533-3668, or 5300-5457, and a VL-CDR3 comprising an amino acid sequence with at least about 100%, 99.9%, 99.8%, 99.7%, 99.6%, 99.5%, 99.4%, 99.3%, 99.2%, 99.1%, 99%, 98%, 97%, 96%, 95%, 94%, 93%, 92%, 91%, 90%, 85%, 80%, 75%, 70%, 65%, 60%, 55%, 50%, 45%, 40%, 35%, 30%, 25%, or at least about 20% sequence identity or sequence similarity to any one of SEQ ID NOs: 2027-2214, 2680-2988, 3669-3804, 4502-4599, 4631-4667, or 5458-5615.

In some embodiments, an anti-EPOR, anti-CD131, or anti-EPO antibody can comprise a VH-CDR1 comprising an amino acid sequence with at most about 100%, 99.9%, 99.8%, 99.7%, 99.6%, 99.5%, 99.4%, 99.3%, 99.2%, 99.1%, 99%, 98%, 97%, 96%, 95%, 94%, 93%, 92%, 91%, 90%, 85%, 80%, 75%, 70%, 65%, 60%, 55%, 50%, 45%, 40%, 35%, 30%, 25%, or at most about 20% sequence identity or sequence similarity to any one of SEQ ID NOs: 1087-1274, 2215-2343, 2989-3124, or 4668-4825, a VH-CDR2 comprising an amino acid sequence with at most about 100%, 99.9%, 99.8%, 99.7%, 99.6%, 99.5%, 99.4%, 99.3%, 99.2%, 99.1%, 99%, 98%, 97%, 96%, 95%, 94%, 93%, 92%, 91%, 90%, 85%, 80%, 75%, 70%, 65%, 60%, 55%, 50%, 45%, 40%, 35%, 30%, 25%, or at most about 20% sequence identity or sequence similarity to any one of SEQ ID NOs: 1275-1462, 2344-2472, 3125-3260, or 4826-4983, a VH-CDR3 comprising an amino acid sequence with at most about 100%, 99.9%, 99.8%, 99.7%, 99.6%, 99.5%, 99.4%, 99.3%, 99.2%, 99.1%, 99%, 98%, 97%, 96%, 95%, 94%, 93%, 92%, 91%, 90%, 85%, 80%, 75%, 70%, 65%, 60%, 55%, 50%, 45%, 40%, 35%, 30%, 25%, or at most about 20% sequence identity or sequence similarity to any one of SEQ ID NOs: 1463-1650, 2473-2601, 3261-3396, 4404-4501, 4600-4630, or 4984-5141, a VL-CDR1 comprising an amino acid sequence with at most about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 95.5%, 96%, 96.5%, 97%, 97.5%, 98%, 98.5%, 99%, or at most about 99.5% sequence identity or sequence similarity to any one of SEQ ID NOs: 1651-1838, 2602-2730, 3397-3532, or 5142-5299, a VL-CDR2 comprising an amino acid sequence with at most about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 95.5%, 96%, 96.5%, 97%, 97.5%, 98%, 98.5%, 99%, or at most about 99.5% sequence identity or sequence similarity to any one of SEQ ID NOs: 1839-2026, 2731-2859, 3533-3668, or 5300-5457, and a VL-CDR3 comprising an amino acid sequence with at most about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 95.5%, 96%, 96.5%, 97%, 97.5%, 98%, 98.5%, 99%, or at most about 99.5% sequence identity or sequence similarity to any one of SEQ ID NOs: 2027-2214, 2680-2988, 3669-3804, 4502-4599, 4631-4667, or 5458-5615.

In some embodiments, an anti-EPOR, anti-CD131, or anti-EPO antibody can comprise a VH-CDR1 comprising an amino acid sequence with about 100%, 99.9%, 99.8%, 99.7%, 99.6%, 99.5%, 99.4%, 99.3%, 99.2%, 99.1%, 99%, 98%, 97%, 96%, 95%, 94%, 93%, 92%, 91%, 90%, 85%, 80%, 75%, 70%, 65%, 60%, 55%, 50%, 45%, 40%, 35%, 30%, 25%, or about 20% sequence identity or sequence similarity to any one of SEQ ID NOs: 1087-1274, 2215-2343, 2989-3124, or 4668-4825, a VH-CDR2 comprising an amino acid sequence with about 100%, 99.9%, 99.8%, 99.7%, 99.6%, 99.5%, 99.4%, 99.3%, 99.2%, 99.1%, 99%, 98%, 97%, 96%, 95%, 94%, 93%, 92%, 91%, 90%, 85%, 80%, 75%, 70%, 65%, 60%, 55%, 50%, 45%, 40%, 35%, 30%, 25%, or about 20% sequence identity or sequence similarity to any one of SEQ ID NOs: 1275-1462, 2344-2472, 3125-3260, or 4826-4983, a VH-CDR3 comprising an amino acid sequence with about 100%, 99.9%, 99.8%, 99.7%, 99.6%, 99.5%, 99.4%, 99.3%, 99.2%, 99.1%, 99%, 98%, 97%, 96%, 95%, 94%, 93%, 92%, 91%, 90%, 85%, 80%, 75%, 70%, 65%, 60%, 55%, 50%, 45%, 40%, 35%, 30%, 25%, or about 20% sequence identity or sequence similarity to any one of SEQ ID NOs: 1463-1650, 2473-2601, 3261-3396, 4404-4501, 4600-4630, or 4984-5141, a VL-CDR1 comprising an amino acid sequence with about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 95.5%, 96%, 96.5%, 97%, 97.5%, 98%, 98.5%, 99%, 99.5%, 99.6%, 99.7%, 99.8%, 99.9%, or about 100% sequence identity or sequence similarity to any one of SEQ ID NOs: 1651-1838, 2602-2730, 3397-3532, or 5142-5299, a VL-CDR2 comprising an amino acid sequence with about 70%, 99.6%, 99.7%, 99.8%, 99.9%, or about 100% sequence identity or sequence similarity to any one of SEQ ID NOs: 1839-2026, 2731-2859, 3533-3668, or 5300-5457, and a VL-CDR3 comprising an amino acid sequence with about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 95.5%, 96%, 96.5%, 97%, 97.5%, 98%, 98.5%, 99%, 99.5%, 99.6%, 99.7%, 99.8%, 99.9%, or about 100% sequence identity or sequence similarity to any one of SEQ ID NOs: 2027-2214, 2680-2988, 3669-3804, 4502-4599, 4631-4667, or 5458-5615.

In some embodiments, an anti-EPOR, anti-CD131, or anti-EPO antibody can comprise a VH-CDR1 comprising an amino acid sequence of any one of SEQ ID NOs: 1087-1274, 2215-2343, 2989-3124, or 4668-4825, or a variant thereof, a VH-CDR2 comprising an amino acid sequence of any one of SEQ ID NOs: 1275-1462, 2344-2472, 3125-3260, or 4826-4983, or a variant thereof, a VH-CDR3 comprising an amino acid sequence of any one of SEQ ID NOs: 1463-1650, 2473-2601, 3261-3396, 4404-4501, 4600-4630, or 4984-5141, or a variant thereof, a VL-CDR1 comprising an amino acid sequence of any one of SEQ ID NOs: 1651-1838, 2602-2730, 3397-3532, or 5142-5299, or a variant thereof, a VL-CDR2 comprising an amino acid sequence of any one of SEQ ID NOs: 1839-2026, 2731-2859, 3533-3668, or 5300-5457, or a variant thereof, and a VL-CDR3 comprising an amino acid sequence of any one of SEQ ID NOs: 2027-2214, 2680-2988, 3669-3804, 4502-4599, 4631-4667, or 5458-5615, or a variant thereof. In some embodiments, a variant can comprise a conservatively modified or substituted amino acid sequence variant.

In some embodiments, an anti-EPOR antibody, anti-CD131 antibody, or anti-EPO antibody can comprise a VH sequence and a kappa chain variable regions (VK) sequence. In some embodiments, an anti-EPOR antibody, anti-CD131 antibody, or anti-EPO antibody can comprise a VH sequence and a lamda chain variable region. In some embodiments, an anti-EPOR antibody, anti-CD131 antibody, or anti-EPO antibody can comprise a VH sequence with at least about 100%, 99.9%, 99.8%, 99.7%, 99.6%, 99.5%, 99.4%, 99.3%, 99.2%, 99.1%, 99%, 98%, 97%, 96%, 95%, 94%, 93%, 92%, 91%, 90%, 85%, 80%, 75%, 70%, 65%, 60%, 55%, 50%, 45%, 40%, 35%, 30%, 25%, or at least about 20% sequence identity or sequence similarity to any one of VH sequences listed in Table 1-1, 10, or 11. In some embodiments, an anti-EPOR antibody, anti-CD131 antibody, or anti-EPO antibody can comprise a VH sequence with at most about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 95.5%, 96%, 96.5%, 97%, 97.5%, 98%, 98.5%, 99%, or at most about 99.5% sequence identity or sequence similarity to any one of VH sequences listed in Table 1-1, 10, or 11. In some embodiments, an anti-EPOR antibody, anti-CD131 antibody, or anti-EPO antibody can comprise a VH sequence with about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 95.5%, 96%, 96.5%, 97%, 97.5%, 98%, 98.5%, 99%, 99.5%, 99.6%, 99.7%, 99.8%, 99.9%, or about 100% sequence identity or sequence similarity to any one of VH sequences listed in Table 1-1, 10, or 11. In some embodiments, an anti-EPOR antibody, anti-CD131 antibody, or anti-EPO antibody can comprise any one of VH sequences listed in Table 1-1, 10, or 11, or a variant thereof. In some embodiments, a variant can comprise a conservatively modified or substituted amino acid sequence variant.

In some embodiments, an anti-EPOR, anti-CD131, or anti-EPO antibody can comprise a VH sequence of any one of SEQ ID NOs: 1, 1053-1069, or 3805-3809, or a variant thereof. In some embodiments, a variant can comprise a conservatively modified or substituted amino acid sequence variant. For example, an anti-EPOR antibody or an anti-CD131 antibody can comprise a VH sequence with at least about 100%, 99.9%, 99.8%, 99.7%, 99.6%, 99.5%, 99.4%, 99.3%, 99.2%, 99.1%, 99%, 98%, 97%, 96%, 95%, 94%, 93%, 92%, 91%, 90%, 85%, 80%, 75%, 70%, 65%, 60%, 55%, 50%, 45%, 40%, 35%, 30%, 25%, or at least about 20% sequence identity or sequence similarity to any one of SEQ ID NOs: 1, 1053-1069, or 3805-3809. In some embodiments, an anti-EPOR, anti-CD131, or anti-EPO antibody can comprise a VH sequence with at most about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 95.5%, 96%, 96.5%, 97%, 97.5%, 98%, 98.5%, 99%, or at most about 99.5% sequence identity or sequence similarity to any one of SEQ ID NOs: 1, 1053-1069, or 3805-3809. In some embodiments, an anti-EPOR, anti-CD131, or anti-EPO antibody can comprise a VH sequence with about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 95.5%, 96%, 96.5%, 97%, 97.5%, 98%, 98.5%, 99%, 99.5%, 99.6%, 99.7%, 99.8%, 99.9%, or about 100% sequence identity or sequence similarity to any one of SEQ ID NOs: 1, 1053-1069, or 3805-3809.

In some embodiments, an anti-EPOR, anti-CD131, or anti-EPO antibody can comprise a VH-CDR1 amino acid sequence derived from a VH set forth in any one of SEQ ID NOs: 1, 1053-1069, or 3805-3809, a VH-CDR2 amino acid sequence derived from a VH set forth in any one of SEQ ID NOs: 1, 1053-1069, or 3805-3809, and a VH-CDR3 amino acid sequence derived from a VH set forth in any one of SEQ ID NOs: 1, 1053-1069, or 3805-3809. In some embodiments, VH-CDR1, VH-CDR2, and/or VH-CDR3 can be defined according to Kabat, Chothia, IMGT, AbM, or Contact.

In some embodiments, an anti-EPOR antibody, anti-CD131 antibody, or anti-EPO antibody can comprise a VK sequence with at least about 100%, 99.9%, 99.8%, 99.7%, 99.6%, 99.5%, 99.4%, 99.3%, 99.2%, 99.1%, 99%, 98%, 97%, 96%, 95%, 94%, 93%, 92%, 91%, 90%, 85%, 80%, 75%, 70%, 65%, 60%, 55%, 50%, 45%, 40%, 35%, 30%, 25%, or at least about 20% sequence identity or sequence similarity to any one of VK sequences listed in Table 1-1, 10, or 11. In some embodiments, an anti-EPOR antibody, anti-CD131 antibody, or anti-EPO antibody can comprise a VK sequence with at most about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 95.5%, 96%, 96.5%, 97%, 97.5%, 98%, 98.5%, 99%, or at most about 99.5% sequence identity or sequence similarity to any one of VK sequences listed in Table 1-1, 10, or 11. In some embodiments, an anti-EPOR antibody, anti-CD131 antibody, or anti-EPO antibody can comprise a VK sequence with about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 95.5%, 96%, 96.5%, 97%, 97.5%, 98%, 98.5%, 99%, 99.5%, 99.6%, 99.7%, 99.8%, 99.9%, or about 100% sequence identity or sequence similarity to any one of VK sequences listed in Table 1-1, 10, or 11. In some embodiments, an anti-EPOR antibody, anti-CD131 antibody, or anti-EPO antibody can comprise any one of VK sequences listed in Table 1-1, 10, or 11, or a variant thereof. In some embodiments, a variant can comprise a conservatively modified or substituted amino acid sequence variant.

In some embodiments, an anti-EPOR antibody, anti-CD131 antibody, or anti-EPO antibody can comprise a VK sequence of any one of SEQ ID NOs: 2, 1070-1086, or 3810-3814, or a variant thereof. In some embodiments, a variant can comprise a conservatively modified or substituted amino acid sequence variant. For example, an anti-EPOR antibody, anti-CD131 antibody, or anti-EPO antibody can comprise a VK sequence with at least about 100%, 99.9%, 99.8%, 99.7%, 99.6%, 99.5%, 99.4%, 99.3%, 99.2%, 99.1%, 99%, 98%, 97%, 96%, 95%, 94%, 93%, 92%, 91%, 90%, 85%, 80%, 75%, 70%, 65%, 60%, 55%, 50%, 45%, 40%, 35%, 30%, 25%, or at least about 20% sequence identity or sequence similarity to any one of SEQ ID NOs: 2, 1070-1086, or 3810-3814. In some embodiments, an anti-EPOR antibody, anti-CD131 antibody, or anti-EPO antibody can comprise a VK sequence with at most about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 95.5%, 96%, 96.5%, 97%, 97.5%, 98%, 98.5%, 99%, or at most about 99.5% sequence identity or sequence similarity to any one of SEQ ID NOs: 2, 1070-1086, or 3810-3814. In some embodiments, an anti-EPOR antibody, anti-CD131 antibody, or anti-EPO antibody can comprise a VK sequence with about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 95.5%, 96%, 96.5%, 97%, 97.5%, 98%, 98.5%, 99%, 99.5%, 99.6%, 99.7%, 99.8%, 99.9%, or about 100% sequence identity or sequence similarity to any one of SEQ ID NOs: 2, 1070-1086, or 3810-3814.

In some embodiments, an anti-EPOR, anti-CD131, or anti-EPO antibody can comprise a VL-CDR1 amino acid sequence derived from a VL set forth in any one of SEQ ID NOs: 2, 1070-1086, or 3810-3814, a VL-CDR2 amino acid sequence derived from a VL set forth in any one of SEQ ID NOs: 2, 1070-1086, or 3810-3814, and a VL-CDR3 amino acid sequence derived from a VL set forth in any one of SEQ ID NOs: 2, 1070-1086, or 3810-3814. In some embodiments, VL-CDR1, VL-CDR2, and/or VL-CDR3 can be defined according to Kabat, Chothia, IMGT, AbM, or Contact.

In some embodiments, an anti-EPOR antibody, anti-CD131 antibody, or anti-EPO antibody can comprise a VH sequence with at least about 100%, 99.9%, 99.8%, 99.7%, 99.6%, 99.5%, 99.4%, 99.3%, 99.2%, 99.1%, 99%, 98%, 97%, 96%, 95%, 94%, 93%, 92%, 91%, 90%, 85%, 80%, 75%, 70%, 65%, 60%, 55%, 50%, 45%, 40%, 35%, 30%, 25%, or at least about 20% sequence identity or sequence similarity to any one of VH sequences listed in Table 1-1, 10, or 11 and a VK sequence with at least about 100%, 99.9%, 99.8%, 99.7%, 99.6%, 99.5%, 99.4%, 99.3%, 99.2%, 99.1%, 99%, 98%, 97%, 96%, 95%, 94%, 93%, 92%, 91%, 90%, 85%, 80%, 75%, 70%, 65%, 60%, 55%, 50%, 45%, 40%, 35%, 30%, 25%, or at least about 20% sequence identity or sequence similarity to any one of VH sequences listed in Table 1-1, 10, or 11. In some embodiments, an anti-EPOR antibody, anti-CD131 antibody, or anti-EPO antibody can comprise a VH sequence with at most about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 95.5%, 96%, 96.5%, 97%, 97.5%, 98%, 98.5%, 99%, or at most about 99.5% sequence identity or sequence similarity to any one of VH sequences listed in Table 1-1, 10, or 11 and a VK sequence with at most about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 95.5%, 96%, 96.5%, 97%, 97.5%, 98%, 98.5%, 99%, or at most about 99.5% sequence identity or sequence similarity to any one of VH sequences listed in Table 1-1, 10, or 11. In some embodiments, an anti-EPOR antibody, anti-CD131 antibody, or anti-EPO antibody can comprise a VH sequence with a about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 95.5%, 96%, 96.5%, 97%, 97.5%, 98%, 98.5%, 99%, 99.5%, 99.6%, 99.7%, 99.8%, 99.9%, or about 100% sequence identity or sequence similarity to any one of VH sequences listed in Table 1-1, 10, or 11 and a VK sequence with about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 95.5%, 96%, 96.5%, 97%, 97.5%, 98%, 98.5%, 99%, 99.5%, 99.6%, 99.7%, 99.8%, 99.9%, or about 100% sequence identity or sequence similarity to any one of VH sequences listed in Table 1-1, 10, or 11. In some embodiments, an anti-EPOR antibody, anti-CD131 antibody, or anti-EPO antibody can comprise any one of VH sequences listed in Table 1-1, 10, or 11, or a variant thereof and any one of VK sequences listed in Table 1-1, 10, or 11, or a variant thereof. In some embodiments, a variant can comprise a conservatively modified or substituted amino acid sequence variant.

In some embodiments, an anti-EPOR, anti-CD131, or anti-EPO antibody can comprise a VH sequence with at least about 100%, 99.9%, 99.8%, 99.7%, 99.6%, 99.5%, 99.4%, 99.3%, 99.2%, 99.1%, 99%, 98%, 97%, 96%, 95%, 94%, 93%, 92%, 91%, 90%, 85%, 80%, 75%, 70%, 65%, 60%, 55%, 50%, 45%, 40%, 35%, 30%, 25%, or at least about 20% sequence identity or sequence similarity to any one of SEQ ID NOs: 1, 1053-1069, or 3805-3809 and a VK sequence with at least about 100%, 99.9%, 99.8%, 99.7%, 99.6%, 99.5%, 99.4%, 99.3%, 99.2%, 99.1%, 99%, 98%, 97%, 96%, 95%, 94%, 93%, 92%, 91%, 90%, 85%, 80%, 75%, 70%, 65%, 60%, 55%, 50%, 45%, 40%, 35%, 30%, 25%, or at least about 20% sequence identity or sequence similarity to any one of SEQ ID NOs: 2, 1070-1086, or 3810-3814. In some embodiments, an anti-EPOR, anti-CD131, or anti-EPO antibody can comprise a VH sequence with at most about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 95.5%, 96%, 96.5%, 97%, 97.5%, 98%, 98.5%, 99%, or at most about 99.5% sequence identity or sequence similarity to any one of SEQ ID NOs: 1, 1053-1069, or 3805-3809 and a VK sequence with at most about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 95.5%, 96%, 96.5%, 97%, 97.5%, 98%, 98.5%, 99%, or at most about 99.5% sequence identity or sequence similarity to any one of SEQ ID NOs: 2, 1070-1086, or 3810-3814. In some embodiments, an anti-EPOR, anti-CD131, or anti-EPO antibody can comprise a VH sequence with a about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 95.5%, 96%, 96.5%, 97%, 97.5%, 98%, 98.5%, 99%, 99.5%, 99.6%, 99.7%, 99.8%, 99.9%, or about 100% sequence identity or sequence similarity to any one of SEQ ID NOs: 1, 1053-1069, or 3805-3809 and a VK sequence with about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 95.5%, 96%, 96.5%, 97%, 97.5%, 98%, 98.5%, 99%, 99.5%, 99.6%, 99.7%, 99.8%, 99.9%, or about 100% sequence identity or sequence similarity to any one of SEQ ID NOs: 2, 1070-1086, or 3810-3814. In some embodiments, an anti-EPOR, anti-CD131, or anti-EPO antibody can comprise a VH sequence of any one of SEQ ID NOs: 1, 1053-1069, or 3805-3809, or a variant thereof and a VK sequence of any one of SEQ ID NOs: 2, 1070-1086, or 3810-3814, or a variant thereof. In some embodiments, a variant can comprise a conservatively modified or substituted amino acid sequence variant.

In some embodiments, an anti-EPOR, anti-CD131, or anti-EPO antibody can comprise a VH-CDR1 amino acid sequence derived from a VH set forth in any one of SEQ ID NOs: 1, 1053-1069, or 3805-3809, a VH-CDR2 amino acid sequence derived from a VH set forth in any one of SEQ ID NOs: 1, 1053-1069, or 3805-3809, a VH-CDR3 amino acid sequence derived from a VH set forth in any one of SEQ ID NOs: 1, 1053-1069, or 3805-3809, a VL-CDR1 amino acid sequence derived from a VL set forth in any one of SEQ ID NOs: 2, 1070-1086, or 3810-3814, a VL-CDR2 amino acid sequence derived from a VL set forth in any one of SEQ ID NOs: 2, 1070-1086, or 3810-3814, and a VL-CDR3 amino acid sequence derived from a VL set forth in any one of SEQ ID NOs: 2, 1070-1086, or 3810-3814. In some embodiments, VH-CDR1, VH-CDR2, VH-CDR3 VL-CDR1, VL-CDR2, and/or VL-CDR3 can be defined according to Kabat, Chothia, IMGT, AbM, or Contact.

In some embodiments, a VH-CDR3 can comprise an amino acid sequence with at least about 100%, 99.9%, 99.8%, 99.7%, 99.6%, 99.5%, 99.4%, 99.3%, 99.2%, 99.1%, 99%, 98%, 97%, 96%, 95%, 94%, 93%, 92%, 91%, 90%, 85%, 80%, 75%, 70%, 65%, 60%, 55%, 50%, 45%, 40%, 35%, 30%, 25%, or at least about 20% sequence identity or sequence similarity to any one of VH-CDR3 sequences listed in Table 12. In some embodiments, a VH-CDR3 can comprise an amino acid sequence with at most about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 95.5%, 96%, 96.5%, 97%, 97.5%, 98%, 98.5%, 99%, or at most about 99.5% sequence identity or sequence similarity to any one of VH-CDR3 sequences listed in Table 12. In some embodiments, a VH-CDR3 can comprise an amino acid sequence with about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 95.5%, 96%, 96.5%, 97%, 97.5%, 98%, 98.5%, 99%, 99.5%, 99.6%, 99.7%, 99.8%, 99.9%, or about 100% sequence identity or sequence similarity to any one of VH-CDR3 sequences listed in Table 12. In some embodiments, a VH-CDR3 can comprise any one of VH-CDR3 sequences listed in Table 12, or a variant thereof. In some embodiments, a VH-CDR3 can comprise an amino acid sequence with at least about 100%, 99.9%, 99.8%, 99.7%, 99.6%, 99.5%, 99.4%, 99.3%, 99.2%, 99.1%, 99%, 98%, 97%, 96%, 95%, 94%, 93%, 92%, 91%, 90%, 85%, 80%, 75%, 70%, 65%, 60%, 55%, 50%, 45%, 40%, 35%, 30%, 25%, or at least about 20% sequence identity or sequence similarity to any one of SEQ ID NOs: 3815-3819. In some embodiments, a VH-CDR3 can comprise an amino acid sequence with at most about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 95.5%, 96%, 96.5%, 97%, 97.5%, 98%, 98.5%, 99%, or at most about 99.5% sequence identity or sequence similarity to any one of SEQ ID NOs: 3815-3819. In some embodiments, a VH-CDR3 can comprise an amino acid sequence with about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 95.5%, 96%, 96.5%, 97%, 97.5%, 98%, 98.5%, 99%, 99.5%, 99.6%, 99.7%, 99.8%, 99.9%, or about 100% sequence identity or sequence similarity to any one of SEQ ID NOs: 3815-3819. In some embodiments, a VH-CDR3 can comprise an amino acid sequence of any one of SEQ ID NOs: 3815-3819, or a variant thereof. In some embodiments, an anti-EPOR or an anti-CD131 antibody that binds to EPO receptor subunit, CD131 subunit, or both subunits of a hetero-EPOR can comprise a VH-CDR3 sequence with at least about 100%, 99.9%, 99.8%, 99.7%, 99.6%, 99.5%, 99.4%, 99.3%, 99.2%, 99.1%, 99%, 98%, 97%, 96%, 95%, 94%, 93%, 92%, 91%, 90%, 85%, 80%, 75%, 70%, 65%, 60%, 55%, 50%, 45%, 40%, 35%, 30%, 25%, or at least about 20% sequence identity or sequence similarity to any one of VH-CDR3 sequences listed in Table 12. In some embodiments, an anti-EPOR or an anti-CD131 antibody that binds to EPO receptor subunit, CD131 subunit, or both subunits of a hetero-EPOR can comprise a VH-CDR3 sequence with at most about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 95.5%, 96%, 96.5%, 97%, 97.5%, 98%, 98.5%, 99%, or at most about 99.5% sequence identity or sequence similarity to any one of VH-CDR3 sequences listed in Table 12. In some embodiments, an anti-EPOR or an anti-CD131 antibody that binds to EPO receptor subunit, CD131 subunit, or both subunits of a hetero-EPOR can comprise a VH-CDR3 sequence with about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 95.5%, 96%, 96.5%, 97%, 97.5%, 98%, 98.5%, 99%, 99.5%, 99.6%, 99.7%, 99.8%, 99.9%, or about 100% sequence identity or sequence similarity to any one of VH-CDR3 sequences listed in Table 12. In some embodiments, an anti-EPOR or an anti-CD131 antibody that binds to EPO receptor subunit, CD131 subunit, or both subunits of a hetero-EPOR can comprise any one of VH-CDR3 sequences listed in Table 12, or a variant thereof. In some embodiments, a variant can comprise a conservatively modified or substituted amino acid sequence variant.

In some embodiments, a VL-CDR3 can comprise an amino acid sequence with at least about 100%, 99.9%, 99.8%, 99.7%, 99.6%, 99.5%, 99.4%, 99.3%, 99.2%, 99.1%, 99%, 98%, 97%, 96%, 95%, 94%, 93%, 92%, 91%, 90%, 85%, 80%, 75%, 70%, 65%, 60%, 55%, 50%, 45%, 40%, 35%, 30%, 25%, or at least about 20% sequence identity or sequence similarity to any one of VL-CDR3 sequences listed in Table 12. In some embodiments, a VL-CDR3 can comprise an amino acid sequence with at most about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 95.5%, 96%, 96.5%, 97%, 97.5%, 98%, 98.5%, 99%, or at most about 99.5% sequence identity or sequence similarity to any one of VL-CDR3 sequences listed in Table 12. In some embodiments, a VL-CDR3 can comprise an amino acid sequence with about 70%, 99.6%, 99.7%, 99.8%, 99.9%, or about 100% sequence identity or sequence similarity to any one of VL-CDR3 sequences listed in Table 12. In some embodiments, a VL-CDR3 can comprise any one of VL-CDR3 sequences listed in Table 12, or a variant thereof. In some embodiments, a VL-CDR3 can comprise an amino acid sequence with at least about 100%, 99.9%, 99.8%, 99.7%, 99.6%, 99.5%, 99.4%, 99.3%, 99.2%, 99.1%, 99%, 98%, 97%, 96%, 95%, 94%, 93%, 92%, 91%, 90%, 85%, 80%, 75%, 70%, 65%, 60%, 55%, 50%, 45%, 40%, 35%, 30%, 25%, or at least about 20% sequence identity or sequence similarity to any one of SEQ ID NOs: 3820-3823. In some embodiments, a VL-CDR3 can comprise an amino acid sequence with at most about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 95.5%, 96%, 96.5%, 97%, 97.5%, 98%, 98.5%, 99%, or at most about 99.5% sequence identity or sequence similarity to any one of SEQ ID NOs: 3820-3823. In some embodiments, a VL-CDR3 can comprise an amino acid sequence with about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 95.5%, 96%, 96.5%, 97%, 97.5%, 98%, 98.5%, 99%, 99.5%, 99.6%, 99.7%, 99.8%, 99.9%, or about 100% sequence identity or sequence similarity to any one of SEQ ID NOs: 3820-3823. In some embodiments, a VL-CDR3 can comprise an amino acid sequence of any one of SEQ ID NOs: 3820-3823, or a variant thereof. In some embodiments, an anti-EPOR or an anti-CD131 antibody that binds to EPO receptor subunit, CD131 subunit, or both subunits of a hetero-EPOR can comprise a VL-CDR3 sequence with at least about 100%, 99.9%, 99.8%, 99.7%, 99.6%, 99.5%, 99.4%, 99.3%, 99.2%, 99.1%, 99%, 98%, 97%, 96%, 95%, 94%, 93%, 92%, 91%, 90%, 85%, 80%, 75%, 70%, 65%, 60%, 55%, 50%, 45%, 40%, 35%, 30%, 25%, or at least about 20% sequence identity or sequence similarity to any one of VL-CDR3 sequences listed in Table 12. In some embodiments, an anti-EPOR or an anti-CD131 antibody that binds to EPO receptor subunit, CD131 subunit, or both subunits of a hetero-EPOR can comprise a VL-CDR3 sequence with at most about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 95.5%, 96%, 96.5%, 97%, 97.5%, 98%, 98.5%, 99%, or at most about 99.5% sequence identity or sequence similarity to any one of VL-CDR3 sequences listed in Table 12. In some embodiments, an anti-EPOR or an anti-CD131 antibody that binds to EPO receptor subunit, CD131 subunit, or both subunits of a hetero-EPOR can comprise a VL-CDR3 sequence with about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 95.5%, 96%, 96.5%, 97%, 97.5%, 98%, 98.5%, 99%, 99.5%, 99.6%, 99.7%, 99.8%, 99.9%, or about 100% sequence identity or sequence similarity to any one of VL-CDR3 sequences listed in Table 12. In some embodiments, an anti-EPOR or an anti-CD131 antibody that binds to EPO receptor subunit, CD131 subunit, or both subunits of a hetero-EPOR can comprise any one of VL-CDR3 sequences listed in Table 12, or a variant thereof. In some embodiments, a variant can comprise a conservatively modified or substituted amino acid sequence variant.

In some embodiments, an anti-EPOR, anti-CD131, or anti-EPO antibody can comprise a VH-CDR1 amino acid sequence derived from a VH set forth in any one of SEQ ID NOs: 3805-3809, a VH-CDR2 amino acid sequence derived from a VH set forth in any one of SEQ ID NOs: 3805-3809, a VH-CDR3 amino acid sequence set forth in any one of SEQ ID NOs: 3815-3819, a VL-CDR1 amino acid sequence derived from a VL set forth in any one of SEQ ID NOs: 3810-3814, a VL-CDR2 amino acid sequence derived from a VL set forth in any one of SEQ ID NOs: 3810-3814, and a VL-CDR3 amino acid sequence set forth in any one of SEQ ID NOs: 3820-3823. In some embodiments, VH-CDR1, VH-CDR2, VH-CDR3 VL-CDR1, VL-CDR2, and/or VL-CDR3 can be defined according to Kabat, Chothia, IMGT, AbM, or Contact.

In some aspects, an anti-EPOR antibody, anti-CD131 antibody, or an anti-EPO antibody can bind to EPOR or EPO with an affinity of from about 1 pM to about 100 nM, from about 2.0 to about 5.1 nM, from about 45 nM to about 300 nM, or from about 2.0 to about 300 nM. In some embodiments, an anti-EPOR antibody, anti-CD131 antibody, or an anti-EPO antibody can bind with an affinity of at least about 300 nM, at least about 140 nM, at least about 100 nM, at least about 5.1 nm, at least about 3.8 nM, or at least about 2.4 nM. In some aspects, a binding affinity can be measured using any method known in the art. For example, a binding affinity can be measure using surface plasmon resonance (SPR; Biacore™, real time molecular interaction monitoring system for analysis of affinity and/or kinetics), KinExA™ Biosensor (system for measuring binding affinity K_{D}), scintillation proximity assays, enzyme-linked immunosorbent assay (ELISA), ORIGEN immunoassay (IGEN), fluorescence quenching, fluorescence transfer, yeast display, or any combination thereof. In some embodiments, a binding affinity can be screened using a suitable bioassay.

In some embodiments, anti-EPO antibodies, anti-EPOR antibodies, or anti-CD131 antibodies described herein can have a binding affinity of less than about 600 nM, about 590 nM, about 580 nM, about 570 nM, about 560 nM, about 550 nM, about 540 nM, about 530 nM, about 520 nM, about 510 nM, about 500 nM, about 490 nM, about 480 nM, about 470 nM, about 460 nM, about 450 nM, about 440 nM, about 430 nM, about 420 nM, about 410 nM, about 400 nM, about 390 nM, about 380 nM, about 370 nM, about 360 nM, about 350 nM, about 340 nM, about 330 nM, about 320 nM, about 310 nM, about 300 nM, about 290 nM, about 280 nM, about 270 nM, about 260 nM, about 250 nM, about 240 nM, about 230 nM, about 220 nM, about 210 nM, about 200 nM, about 190 nM, about 180 nM, about 170 nM, about 160 nM, about 150 nM, about 140 nM, about 130 nM, about 120 nM, about 110 nM, about 100 nM, about 90 nM, about 80 nM, about 70 nM, about 50 nM, about 50 nM, about 49 nM, about 48 nM, about 47 nM, about 46 nM, about 45 nM, about 44 nM, about 43 nM, about 42 nM, about 41 nM, about 40 nM, about 39 nM, about 38 nM, about 37 nM, about 36 nM, about 35 nM, about 34 nM, about 33 nM, about 32 nM, about 31 nM, about 30 nM, about 29 nM, about 28 nM, about 27 nM, about 26 nM, about 25 nM, about 24 nM, about 23 nM, about 22 nM, about 21 nM, about 20 nM, about 19 nM, about 18 nM, about 17 nM, about 16 nM, about 15 nM, about 14 nM, about 13 nM, about 12 nM, about 11 nM, about 10 nM, about 9 nM, about 8 nM, about 7 nM, about 6 nM, about 5 nM, about 4 nM, about 3 nM, about 2 nM, about 1 nM, about 990 pM, about 980 pM, about 970 pM, about 960 pM, about 950 pM, about 940 pM, about 930 pM, about 920 pM, about 910 pM, about 900 pM, about 890 pM, about 880 pM, about 870 pM, about 860 pM, about 850 pM, about 840 pM, about 830 pM, about 820 pM, about 810 pM, about 800 pM, about 790 pM, about 780 pM, about 770 pM, about 760 pM, about 750 pM, about 740 pM, about 730 pM, about 720 pM, about 710 pM, about 700 pM, about 690 pM, about 680 pM, about 670 pM, about 660 pM, about 650 pM, about 640 pM, about 630 pM, about 620 pM, about 610 pM, about 600 pM, about 590 pM, about 580 pM, about 570 pM, about 560 pM, about 550 pM, about 540 pM, about 530 pM, about 520 pM, about 510 pM, about 500 pM, about 490 pM, about 480 pM, about 470 pM, about 460 pM, about 450 pM, about 440 pM, about 430 pM, about 420 pM, about 410 pM, about 400 pM, about 390 pM, about 380 pM, about 370 pM, about 360 pM, about 350 pM, about 340 pM, about 330 pM, about 320 pM, about 310 pM, about 300 pM, about 290 pM, about 280 pM, about 270 pM, about 260 pM, about 250 pM, about 240 pM, about 230 pM, about 220 pM, about 210 pM, about 200 pM, about 190 pM, about 180 pM, about 170 pM, about 160 pM, or any integer therebetween. In some embodiments, anti-EPO antibodies, anti-EPOR antibodies, or anti-CD131 antibodies described herein can have a binding affinity of less than 150 pM, about 140 pM, about 130 pM, about 120 pM, about 110 pM, about 100 pM, about 95 pM, about 90 pM, about 85 pM, about 80 pM, about 75 pM, about 70 pM, about 65 pM, about 60 pM, about 55 pM, about 50 pM about 45 pM, about 40 pM, about 35 pM, about 30 pM, about 25 pM, about 20 pM, about 15 pM, about 10 pM, about 9 pM, about 8 pM, about 7 pM, about 6 pM, about 5 pM, about 4 pM, about 3 pM, about 2 pM, about 1 pM, about 0.9 pM, about 0.8 pM, about 0.7 pM, about 0.6 pM, about 0.5 pM, about 0.4 pM, about 0.3 pM, about 0.2 pM, about 0.1 pM, about 0.09 pM, about 0.08, about 0.07 pM, about 0.06 pM, about 0.05 pM, about 0.04 pM, about 0.03 pM, about 0.02 pM, about 0.01 pM, or any integer therebetween.

In some instances, anti-EPO antibodies, anti-EPOR antibodies, or anti-CD131 antibodies described herein can bind to EPO, EPOR, or CD131 from a subject other than human. For example, anti-EPO antibodies, anti-EPOR antibodies, or anti-CD131 antibodies described herein can bind to EPO, EPOR, or CD131 from mouse or cynomolgus monkey. In some instances, optical density (OD) values can be used to describe antigen binding of anti-EPO antibodies, anti-EPOR antibodies, anti-CD131 antibodies described herein. For example, OD can be used to calculate the concentration of antigens or antigen binding in a sample for ELISA analysis. In some embodiments, anti-EPO antibodies, anti-EPOR antibodies, or anti-CD131 antibodies described herein can have OD450 value of from about 0.0150 to about 5.000, when analyzed by ELISA. In some embodiments, anti-EPO antibodies, anti-EPOR antibodies, anti-CD131 antibodies described herein can have OD450 value of from about 0.0150 to about 5.000 for human EPOR/CD131-Fc binding, when analyzed by ELISA. In some embodiments, anti-EPO antibodies, anti-EPOR antibodies, anti-CD131 antibodies described herein can have OD450 value of from about 0.400 to about 5.000 for mouse EPOR/CD131-Fc binding, when analyzed by ELISA. In some embodiments, anti-EPO antibodies, anti-EPOR antibodies, anti-CD131 antibodies described herein can have OD450 value of from about 0.050 to about 5.000 for cynomolgus monkey EPOR/CD131-Fc binding, when analyzed by ELISA. In some embodiments, anti-EPO antibodies, anti-EPOR antibodies, or anti-CD131 antibodies described herein can have OD450 value of from about 0.070 to about 5.000 for human EPOR-Fc binding, when analyzed by ELISA. In some embodiments, anti-EPO antibodies, anti-EPOR antibodies, or anti-CD131 antibodies described herein can have OD450 value of from about 0.060 to about 5.000 for human CD131-Fc binding, when analyzed by ELISA. In some embodiments, anti-EPO antibodies, anti-EPOR antibodies, or anti-CD131 antibodies described herein can have OD450 value of from about 0.100 to about 5.000 for cynomolgus monkey EPOR-Fc binding, when analyzed by ELISA.

In some instances, anti-EPO antibodies, anti-EPOR antibodies, or anti-CD131 antibodies described herein can have antagonistic effects. In some embodiments, anti-EPO antibodies described herein can bind EPOs and inhibit or block EPO/EPOR interaction. For example, anti-EPO antibodies can bind EPOs and inhibit EPOs from binding to homo-EPORs or hetero-EPORs. In some embodiments, anti-EPO antibodies, anti-EPOR antibodies, or anti-CD131 antibodies described herein can bind an EPOR subunit of homo-EPORs or hetero-EPORs and inhibit or block homo-EPOR complex formation, hetero-EPOR complex formation, EPO/homo-EPOR interaction, or EPO/hetero-EPOR interaction. For example, anti-EPO antibodies, anti-EPOR antibodies, or anti-CD131 antibodies described herein can bind an EPOR subunit and inhibit formation of homo-EPORs or hetero-EPORs. For example, anti-EPO antibodies, anti-EPOR antibodies, or anti-CD131 antibodies described herein can bind an EPOR subunit of homo-EPORs or hetero-EPORs and inhibit homo-EPORs or hetero-EPORs from binding to EPO. In some embodiments, anti-EPO antibodies, anti-EPOR antibodies, or anti-CD131 antibodies described herein can bind a CD131 and inhibit or block hetero-EPOR complex formation or EPO/hetero-EPOR interaction. For example, anti-EPO antibodies, anti-EPOR antibodies, or anti-CD131 antibodies described herein can bind a CD131 and inhibit formation of hetero-EPORs. For example, anti-EPO antibodies, anti-EPOR antibodies, or anti-CD131 antibodies described herein can bind a CD131 subunit of hetero-EPORs and inhibit hetero-EPORs from binding to EPO. In some embodiments, preventing or inhibiting complex formation of a homo-EPOR or complex formation between an EPO protein and a homo-EPOR can lead to prevention or inhibition of homo-EPOR activation or function. In some embodiments, preventing or inhibiting complex formation of a hetero-EPOR or complex formation between an EPO protein and a hetero-EPOR can lead to prevention or inhibition of hetero-EPOR activation or function. In some embodiments, the level of prevention or inhibition is at least about 1%, at least about 5%, at least about 10%, at least about 15%, at least about 20%, at least about 25%, at least about 30%, at least about 35%, at least about 40, at least about 45%, at least about 50%, at least about 55%, at least about 60%, at least about 65%, at least about 70%, at least about 71%, at least about 72%, at least about 73%, at least about 74%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or at least about 100%.

In some embodiments, anti-EPO antibodies, anti-EPOR antibodies, or anti-CD131 antibodies described herein can bind an EPO protein and inhibit or decrease the level of an activity of a homo-EPOR or a hetero-EPOR without affecting binding of the EPO protein to the homo-EPOR or the hetero-EPOR. In some embodiments, anti-EPO antibodies, anti-EPOR antibodies, or anti-CD131 antibodies described herein can bind to an EPO receptor subunit of a homo-EPOR or a hetero-EPOR and inhibit or decrease the level of an activity of the homo-EPOR or the hetero-EPOR without affecting the complex formation of the homo-EPOR or the hetero-EPOR, or complex formation between an EPO protein and the homo-EPOR or an EPO protein and the hetero-EPOR. In some embodiments, anti-EPO antibodies, anti-EPOR antibodies, or anti-CD131 antibodies described herein can bind a CD131 subunit of a hetero-EPOR and inhibit or decrease the level of an activity of the hetero-EPOR without affecting complex formation of the hetero-EPOR or binding of an EPO protein to the hetero-EPOR.

In some embodiments, affinity maturation can be used with an antibody disclosed herein to obtain an anti-EPOR antibody, anti-CD131, or an anti-EPO antibody of a desired affinity. When an anti-EPOR antibody, anti-CD131, or anti-EPO antibody is obtained from an animal (e.g., a transgenic animal carrying a human antibody repertoire), the antibodies made in the transgenic animal can undergo affinity maturation. Alternatively, antibodies from a transgenic animal, or from other technologies (such as a display technology) can be affinity matured using chain shuffling approaches and/or mutation of the nucleic acids encoding VH and VL followed by screening and/or selecting for antibodies with greater affinity.

The most widely used methods for minimizing the immunogenicity of non-human antibodies while retaining specificity and affinity can involve grafting the CDRs of the non-human antibody onto human frameworks typically selected for their structural homology to the non-human framework (Jones et al., 1986, Nature 321:522-5; U.S. Pat. No. 5,225,539, both of which are hereby incorporated by reference in their entirety). The inclusion of some non-human residues at key positions in the framework can improve the affinity of the CDR grafted antibody (Bajorath et al., 1995, J Biol Chem 270:22081-4; Martin et al., 1991, Methods Enzymol. 203:121-53; Al-Lazikani, 1997, J Mol Biol 273:927-48, all of which are hereby incorporated by reference in their entirety). Exemplary methods for humanization of antibodies by CDR grafting are disclosed, for example, in U.S. Pat. No. 6,180,370, which is hereby incorporated by reference in its entirety.

Improvements to the traditional CDR-grafting approaches can use various hybrid selection approaches, in which portions of the non-human antibody have been combined with libraries of complementary human antibody sequences in successive rounds of selection for antigen binding, in the course of which most of the non-human sequences are gradually replaced with human sequences. For example, in the chain-shuffling technique (Marks, et al., 1992, Biotechnology 10:779-83, which is hereby incorporated by reference in its entirety for all purposes) one chain of the non-human antibody can be combined with a naive human repertoire of the other chain on the rationale that the affinity of the non-human chain will be sufficient to constrain the selection of a human partner to the same epitope on the antigen. Selected human partners can then be used to guide selection of human counterparts for the remaining non-human chains.

Other methodologies can include chain replacement techniques where the non-human CDR3s were retained and only the remainder of the V-regions, including the frameworks and CDRs 1 and 2, were individually replaced in steps performed sequentially (e.g., U.S. patent application No. 20030166871; Rader, et al., Proc Natl Acad Sci USA 95:8910-15, 1998; Steinberger, et al., J. Biol. Chem. 275:36073-36078, 2000; Rader, et al., J. Biol. Chem. 275:13668-13676, 2000, all of which are hereby incorporated by reference in their entirety for all purposes).

These technologies can be used to make antibodies suitable for use in non-human subjects by engineering the CDRs into framework regions of the subject species using analogous approaches to the CDR grafting methods used for making antibodies for use in humans.

The disclosure encompasses pharmaceutically acceptable salts of anti-EPOR antibodies, anti-CD131 antibodies, or anti-EPO antibodies, including those with a positive net charge, those with a negative net charge, and those with no net charge, and including, without limitation, salts of anti-EPOR antibodies, anti-CD131 antibodies, or anti-EPO antibodies including fragments thereof as compounds, in pharmaceutical compositions, in their therapeutic and diagnostic uses, and in their production.

In some embodiments, anti-EPO antibodies, anti-EPOR antibodies, anti-CD131 antibodies described herein can have a half-life of from 1 minute to 1 hour in human plasma. In some embodiments, anti-EPO antibodies, anti-EPOR antibodies, anti-CD131 antibodies described herein can have a half-life of about 1 minute to 2 minutes, about 1 minute to about 4 minutes, about 1 minute to about 5 minutes, about 1 minute to about 10 minutes, about 1 minute to about 15 minutes, about 1 minute to about 20 minutes, about 1 minute to about 25 minutes, about 1 minute to about 30 minutes, about 1 minute to about 35 minutes, about 1 minute to about 40 minutes, about 1 minute to about 45 minutes, about 1 minute to about 50 minutes, about 1 minute to about 55 minutes, or about 1 minute to about 1 hour. In some embodiments, anti-EPO antibodies, anti-EPOR antibodies, anti-CD131 antibodies described herein can have a half-life of about 1 minute, about 2 minutes, about 3 minutes, about 4 minutes, about 5 minutes, about 10 minutes, about 15 minutes, about 20 minutes, about 25 minutes, about 30 minutes, about 35 minutes, about 40 minutes, about 45 minutes, about 50 minutes, about 55 minutes, or about 1 hour. In some embodiments, anti-EPO antibodies, anti-EPOR antibodies, anti-CD131 antibodies described herein can have a half-life of from 1 hour to 5 days in human plasma. In some embodiments, anti-EPO antibodies, anti-EPOR antibodies, anti-CD131 antibodies described herein can have a half-life about 1 hour to about 120 hours. In some embodiments, anti-EPO antibodies, anti-EPOR antibodies, anti-CD131 antibodies described herein can have a half-life about 1 hour to about 5 hours, about 1 hour to about 10 hours, about 1 hour to about 12 hours, about 1 hour to about 24 hours, about 1 hour to about 36 hours, about 1 hour to about 48 hours, about 1 hour to about 60 hours, about 1 hour to about 72 hours, about 1 hour to about 84 hours, about 1 hour to about 96 hours, about 1 hour to about 120 hours, about 5 hours to about 10 hours, about 5 hours to about 12 hours, about 5 hours to about 24 hours, about 5 hours to about 36 hours, about 5 hours to about 48 hours, about 5 hours to about 60 hours, about 5 hours to about 72 hours, about 5 hours to about 84 hours, about 5 hours to about 96 hours, about 5 hours to about 120 hours, about 10 hours to about 12 hours, about 10 hours to about 24 hours, about 10 hours to about 36 hours, about 10 hours to about 48 hours, about 10 hours to about 60 hours, about 10 hours to about 72 hours, about 10 hours to about 84 hours, about 10 hours to about 96 hours, about 10 hours to about 120 hours, about 12 hours to about 24 hours, about 12 hours to about 36 hours, about 12 hours to about 48 hours, about 12 hours to about 60 hours, about 12 hours to about 72 hours, about 12 hours to about 84 hours, about 12 hours to about 96 hours, about 12 hours to about 120 hours, about 24 hours to about 36 hours, about 24 hours to about 48 hours, about 24 hours to about 60 hours, about 24 hours to about 72 hours, about 24 hours to about 84 hours, about 24 hours to about 96 hours, about 24 hours to about 120 hours, about 36 hours to about 48 hours, about 36 hours to about 60 hours, about 36 hours to about 72 hours, about 36 hours to about 84 hours, about 36 hours to about 96 hours, about 36 hours to about 120 hours, about 48 hours to about 60 hours, about 48 hours to about 72 hours, about 48 hours to about 84 hours, about 48 hours to about 96 hours, about 48 hours to about 120 hours, about 60 hours to about 72 hours, about 60 hours to about 84 hours, about 60 hours to about 96 hours, about 60 hours to about 120 hours, about 72 hours to about 84 hours, about 72 hours to about 96 hours, about 72 hours to about 120 hours, about 84 hours to about 96 hours, about 84 hours to about 120 hours, or about 96 hours to about 120 hours. In some embodiments, anti-EPO antibodies, anti-EPOR antibodies, anti-CD131 antibodies described herein can have a half-life about 1 hour, about 5 hours, about 10 hours, about 12 hours, about 24 hours, about 36 hours, about 48 hours, about 60 hours, about 72 hours, about 84 hours, about 96 hours, or about 120 hours. In some embodiments, anti-EPO antibodies, anti-EPOR antibodies, anti-CD131 antibodies described herein can have a half-life at least about 1 hour, about 5 hours, about 10 hours, about 12 hours, about 24 hours, about 36 hours, about 48 hours, about 60 hours, about 72 hours, about 84 hours, or about 96 hours. In some embodiments, anti-EPO antibodies, anti-EPOR antibodies, anti-CD131 antibodies described herein can have a half-life at most about 5 hours, about 10 hours, about 12 hours, about 24 hours, about 36 hours, about 48 hours, about 60 hours, about 72 hours, about 84 hours, about 96 hours, or about 120 hours. In some embodiments, anti-EPO antibodies, anti-EPOR antibodies, anti-CD131 antibodies described herein can have a half-life at least about 10 days, about 11 days, about 11 days, 12 days, 13 days, 14 days, 15 days, 16 days, 17 days, 18 days, 19 days, or 20 days. In some embodiments, anti-EPO antibodies, anti-EPOR antibodies, anti-CD131 antibodies described herein can have a half-life at about 10 days to about 11 days, about 10 to about 12 days, about 10 days to about 13 days, 10 days to about 14 days, about 10 days to about 15 days, about 10 days to about 16 days, about 10 days to about 17 days, about 10 days to about 18 days, about 10 days to about 19 days, or about 10 days to about 20 days. In some embodiments, anti-EPO antibodies, anti-EPOR antibodies, anti-CD131 antibodies described herein can have a half-life at about 14 days to about 17 days.

The disclosure also encompasses bispecific or multispecific antibodies that can have specificity for at least two antigens. For example, anti-EPO antibodies, anti-EPOR antibodies, anti-CD131 antibodies described herein can be generated as bispecific antibodies that can also bind another target. In some embodiments, anti-EPO antibodies, anti-EPOR antibodies, anti-CD131 antibodies described herein can be generated as bispecific antibodies that can also bind a cell surface marker associated with immune cells, a signaling molecule associated with immune cells, or an antigen associated with tumor. In some embodiments, bispecific antibodies described herein can enhance specificity and/or selectivity of anti-EPO, anti-EPOR, anti-CD131 antibodies described herein. For example, bispecific antibodies that can bind a cell surface marker of immune cells and any of EPO, homo-EPOR (e.g., EPOR homodimer comprising two EPORs), or hetero-EPOR (e.g., EPOR heterodimer comprising an EPOR and a CD131) can be used to target EPO, homo-EPOR, or hetero-EPOR in immune cells. For example, bispecific antibodies that can bind a signaling molecule of immune cells and any of EPO, homo-EPOR, or hetero-EPOR can be used to target EPO, homo-EPOR, or hetero-EPOR in immune cells. For example, bispecific antibodies that can bind an antigen associated with tumor and any of EPO, homo-EPOR, or hetero-EPOR can be used to target EPO, homo-EPOR, or hetero-EPOR in tumor or cancer cells.

In some embodiments, a bispecific antibody can bind (i) EPO, EPO receptor subunit of a homo-EPOR or a hetero-EPOR, CD131 subunit of a hetero-EPOR, a homo-EPOR, a hetero EPOR; and (ii) a cell surface marker associated with immune cells. Examples of cell surface markers associated with immune cells can include, but are not limited to, lymphocyte antigen 75 (DEC205), X-C motif chemokine receptor 1 (XCR1), or X-C motif chemokine ligand 1 (XCL1). In some embodiments, bispecific antibodies described herein can enhance specificity and/or selectivity of anti-EPO, anti-EPOR, anti-CD131 antibodies described herein for targeting immune cells. For example, bispecific antibodies that can bind a cell surface marker associated with immune cells and any of EPO, homo-EPOR, hetero-EPOR can be used to target EPO, homo-EPOR, or hetero-EPOR in immune cells. In some embodiments, bispecific antibodies described herein can specifically and/or selectively target EPO, homo-EPOR, or hetero-EPOR in immune cells and specifically and/or selectively increase or decrease homo-EPOR activity or hetero-EPOR activity described herein in immune cells. In some embodiments, immune cells can comprise macrophages, dendritic cells, T-cells, natural killer cells, or B cells.

In some embodiments, a bispecific antibody can bind (i) EPO, EPO receptor subunit of a homo-EPOR or a hetero-EPOR, CD131 subunit of a hetero-EPOR, a homo-EPOR, a hetero EPOR; and (ii) a signaling molecule associated with immune cells. Examples of signaling molecules associated with immune cells can include, but are not limited to, Programmed Death Ligand 1 (PD-L1), T-cell immunoglobulin and mucin-domain containing 3 (Tim3), or Triggering receptor expressed on myeloid cells 2 (TREM2). In some embodiments, bispecific antibodies described herein can enhance specificity and/or selectivity of anti-EPO, anti-EPOR, or anti-CD131 antibodies described herein for targeting immune cells. For example, bispecific antibodies that can bind a signaling molecule associated with immune cells and any of EPO, homo-EPOR, or hetero-EPOR can be used to target EPO, homo-EPOR, or hetero-EPOR in immune cells and can have synergistic anti-cancer effect. In some embodiments, bispecific antibodies described herein can specifically and/or selectively target EPO, homo-EPOR, or hetero-EPOR in immune cells and specifically and/or selectively increase or decrease homo-EPOR activity or hetero-EPOR activity described herein in immune cells. For example, bispecific antibodies described herein can be used to specifically and/or selectively target EPO, homo-EPOR, or hetero-EPOR in immune cells and specifically and/or selectively increase hetero-EPOR activity to stimulate immune response in cancer. In some embodiments, immune cells can comprise macrophages, dendritic cells, T-cells, natural killer cells, or B cells.

In some embodiments, a bispecific antibody can bind (i) EPO, EPO receptor subunit of a homo-EPOR or a hetero-EPOR, CD131 subunit of a hetero-EPOR, a homo-EPOR, a hetero EPOR; and (ii) a tumor marker or an antigen associated with tumor. Examples of tumor markers or antigens associated with tumor can include, but are not limited to, PD1, HER2, CEA, CEACAM5, CD19, CD20, CD22, prostate specific antigen (PSA), CD123, CLL-1, B cell maturation antigen, CD138, CD133 (PROM1), CD44, ALDH1A1, CD34, CD24, EpCAM (ESA), CD117 (KIT), CD90 (THY1), CD166 (ALCAM), PDXL-1, PTCH, CD87 (PLAUR), SSEA-1, EGFR, SP, ALDH, CD49, CD326, LGR5, ALDH1A, LETM1, NANOG, POU5F1, SALL4, SOX2, LINGO2, AFP, NOTCH1, NOTCH2, NOTCH3, CTNNBL1, CD29, CD25, CD61, PROCR, TSPAN8, BMI1, FOXO1, FOXO3, FOXO4, CD15 (FUT4), CHL1, KLF4, NES, TACSTD2, TGM2, CD36, IL1RAP, GLI2, TET2, DNMT3A, KRAS, LDHB, LDHC, LDHD, NPM1, CD33, CD49f, CD171, ABCG2, FZD, CXCR4, OCT4, ALDH, E-cadherin, CD200, ABCB5, vimentin, CD146, CD31, CD144, or CD201 (PROCR). In some embodiments, bispecific antibodies described herein can enhance specificity and/or selectivity of anti-EPO, anti-EPOR, or anti-CD131 antibodies described herein for targeting tumors. For example, bispecific antibodies that can bind a tumor-associated antigen and any of EPO, homo-EPOR, or hetero-EPOR can be used to target EPO, homo-EPOR, or hetero-EPOR in cancer or tumor cells. In some embodiments, tumor-associated antigens can be on cancer or tumor cells (e.g., on cell membrane) or secreted by cancer or tumor cells. In some embodiments, bispecific antibodies described herein can specifically and/or selectively target EPO, homo-EPOR, or hetero-EPOR in cancer or tumor cells and specifically and/or selectively increase or decrease homo-EPOR activity or hetero-EPOR activity described herein in cancer or tumor cells.

In some aspects, provided herein is a bispecific antibody that can bind a first target and a second target. In some embodiments, a bispecific antibody can comprise a first antigen-binding domain and a second antigen-binding domain. In some embodiments, the first or the second antigen-binding domain can comprise a VH comprising a VH-CDR1 sequence, a VH-CDR2 sequence, and a VH-CDR3 sequence; and a VL comprising a VL-CDR1 sequence, a VL-CDR2 sequence, and a VL-CDR3 sequence; a VH and a VK; or a VH and a lambda chain variable regions. In some embodiments, the first or the second antigen-binding domain can comprise a Fab, a Fab′, a (Fab′)2, a variable fragment (Fv), a single chain variable fragment (scFv), a scFv-Fc, a Fab-Fc, a VHH, a non-antibody scaffold, or a combination thereof. In some embodiments, a bispecific antibody can further comprise a linker, e.g., a peptide linker described herein. For example, a bispecific antibody can comprise (i) a heavy chain and a light chain of an anti-EPO antibody, an anti-EPOR antibody, or an anti-CD131 antibody, (ii) a VHH or a nanobody of a second antibody, and optionally (iii) a peptide linker described herein. In some embodiments, a bispecific antibody can comprise (i) a heavy chain and a light chain of an anti-EPO antibody, an anti-EPOR antibody, or an anti-CD131 antibody, (ii) a VHH or a nanobody that can bind to an immune checkpoint protein, and optionally (iii) a peptide linker described herein. In some embodiments, an immune checkpoint protein can comprise PD-1, PD-L1, or CTLA-4. In some embodiments, a bispecific antibody can comprise (i) a heavy chain and a light chain of an anti-EPO antibody, an anti-EPOR antibody, or an anti-CD131 antibody, (ii) an anti-PD-L1 nanobody comprising an amino sequence of SEQ ID NO: 5992 or a variant thereof, and optionally (iii) a peptide linker comprising (GGGGS)₃ (SEQ ID NO: 79) or (GGGGS)₄ (SEQ ID NO: 80). In some embodiments, a variant can comprise a conservatively modified or substituted amino acid sequence variant. In some embodiments, a bispecific antibody described herein can bind to (i) EPO, homo-EPOR, or hetero-EPOR and (ii) an immune checkpoint protein (e.g., PD-1, PD-L1, or CTLA-4, etc.). In some embodiments, binding of the bispecific antibody to an immune checkpoint protein can inhibit immune tolerance.

KN035 nanobody sequence
(SEQ ID NO: 5992):
QVQLQESGGGLVQPGGSLRLSCAASGKMSSRRCMAWFRQAPGKERE
RVAKLLTTSGSTYLADSVKGRFTISQNNAKSTVYLQMNSLKPEDTA
MYYCAADSFEDPTCTLVTSSGAFQYWGQGTQVTVSS

In some embodiments, a bispecific antibody can comprise a heavy chain of an anti-EPO antibody, an anti-EPOR antibody, or an anti-CD131 antibody comprising an amino acid sequence with at least about 100%, 99.9%, 99.8%, 99.7%, 99.6%, 99.5%, 99.4%, 99.3%, 99.2%, 99.1%, 99%, 98%, 97%, 96%, 95%, 94%, 93%, 92%, 91%, 90%, 85%, 80%, 75%, 70%, 65%, 60%, 55%, 50%, 45%, 40%, 35%, 30%, 25%, or at least about 20% sequence identity or sequence similarity to SEQ ID NO: 1, 3, 147-334, 523-651, 781-916, 1053-1069, 3805-3809, 3824-3921, 4020-4050, 4088-4245, or 5616-5803. In some embodiments, a bispecific antibody can comprise a heavy chain of an anti-EPO antibody, an anti-EPOR antibody, or an anti-CD131 antibody comprising an amino acid sequence with at most about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 95.5%, 96%, 96.5%, 97%, 97.5%, 98%, 98.5%, 99%, or at most about 99.5% sequence identity or sequence similarity to SEQ ID NO: 1, 3, 147-334, 523-651, 781-916, 1053-1069, 3805-3809, 3824-3921, 4020-4050, 4088-4245, or 5616-5803. In some embodiments, a bispecific antibody can comprise a heavy chain of an anti-EPO antibody, an anti-EPOR antibody, or an anti-CD131 antibody comprising an amino acid sequence with about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 95.5%, 96%, 96.5%, 97%, 97.5%, 98%, 98.5%, 99%, 99.5%, 99.6%, 99.7%, 99.8%, 99.9%, or about 100% sequence identity or sequence similarity to SEQ ID NO: 1, 3, 147-334, 523-651, 781-916, 1053-1069, 3805-3809, 3824-3921, 4020-4050, 4088-4245, or 5616-5803. In some embodiments, a bispecific antibody can comprise a heavy chain of an anti-EPO antibody, an anti-EPOR antibody, or an anti-CD131 antibody comprising an amino acid sequence of SEQ ID NO: 1, 3, 147-334, 523-651, 781-916, 1053-1069, 3805-3809, 3824-3921, 4020-4050, 4088-4245, or 5616-5803, or a variant thereof. In some embodiments, a variant can comprise a conservatively modified or substituted amino acid sequence variant.

In some embodiments, a bispecific antibody can comprise a light chain of an anti-EPO antibody, an anti-EPOR antibody, or an anti-CD131 antibody comprising an amino acid sequence with at least about 100%, 99.9%, 99.8%, 99.7%, 99.6%, 99.5%, 99.4%, 99.3%, 99.2%, 99.1%, 99%, 98%, 97%, 96%, 95%, 94%, 93%, 92%, 91%, 90%, 85%, 80%, 75%, 70%, 65%, 60%, 55%, 50%, 45%, 40%, 35%, 30%, 25%, or at least about 20% sequence identity or sequence similarity to SEQ ID NO: 2, 4, 335-522, 652-780, 917-1052, 1070-1086, 3810-3814, 3922-4019, 4051-4087, 4246-4403, or 5804-5991. In some embodiments, a bispecific antibody can comprise a light chain of an anti-EPO antibody, an anti-EPOR antibody, or an anti-CD131 antibody comprising an amino acid sequence with at most about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 95.5%, 96%, 96.5%, 97%, 97.5%, 98%, 98.5%, 99%, or at most about 99.5% sequence identity or sequence similarity to SEQ ID NO: 2, 4, 335-522, 652-780, 917-1052, 1070-1086, 3810-3814, 3922-4019, 4051-4087, 4246-4403, or 5804-5991. In some embodiments, a bispecific antibody can comprise a light chain of an anti-EPO antibody, an anti-EPOR antibody, or an anti-CD131 antibody comprising an amino acid sequence with about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 95.5%, 96%, 96.5%, 97%, 97.5%, 98%, 98.5%, 99%, 99.5%, 99.6%, 99.7%, 99.8%, 99.9%, or about 100% sequence identity or sequence similarity to SEQ ID NO: 2, 4, 335-522, 652-780, 917-1052, 1070-1086, 3810-3814, 3922-4019, 4051-4087, 4246-4403, or 5804-5991. In some embodiments, a bispecific antibody can comprise a light chain of an anti-EPO antibody, an anti-EPOR antibody, or an anti-CD131 antibody comprising an amino acid sequence of SEQ ID NO: 2, 4, 335-522, 652-780, 917-1052, 1070-1086, 3810-3814, 3922-4019, 4051-4087, 4246-4403, or 5804-5991, or a variant thereof. In some embodiments, a variant can comprise a conservatively modified or substituted amino acid sequence variant.

The disclosure also encompasses a composition comprising a combination or a population of antibodies or functional fragments thereof described herein. For example, a composition can comprise one, two, three, four, five, six, seven, eight, nine, ten, or more different antibodies or functional fragments thereof. In one embodiment, a composition can comprise one antibody or a functional fragment thereof described herein. In another embodiment, a composition can comprise a combination or a population of antibodies or functional fragments comprising two different antibodies or functional fragments thereof. In another embodiment, a composition can comprise a combination or a population of antibodies or functional fragments thereof comprising three different antibodies or functional fragments thereof. In yet another embodiment, a composition can comprise a combination or a population of antibodies or functional fragments thereof comprising four, five, six, seven, eight, nine, ten, or more than ten different antibodies or functional fragments thereof. In some embodiments, each of the one, two, three, four, five, six, seven, eight, nine, ten, or more different antibodies or functional fragments thereof can bind to the same target (e.g., an EPO protein, an EPO receptor subunit, or a CD131 subunit, etc.). In some embodiments, each of the one, two, three, four, five, six, seven, eight, nine, ten, or more different antibodies or functional fragments thereof can bind to a different part of the same target (e.g., an EPO protein, an EPO receptor subunit, or a CD131 subunit, etc.). In some embodiments, each of the one, two, three, four, five, six, seven, eight, nine, ten, or more different antibodies or functional fragments thereof can bind to a different target (e.g., an EPO protein, an EPO receptor subunit, a CD131 subunit, or a combination thereof). In some embodiments, at least two of the one, two, three, four, five, six, seven, eight, nine, ten, or more different antibodies or functional fragments thereof can bind to the same target (e.g., an EPO protein, an EPO receptor subunit, or a CD131 subunit, etc.) and at least two of the one, two, three, four, five, six, seven, eight, nine, ten, or more different antibodies or functional fragments thereof can bind to a different target (e.g., an EPO protein, an EPO receptor subunit, a CD131 subunit, or a combination thereof). In some embodiments, at least two of the one, two, three, four, five, six, seven, eight, nine, ten, or more different antibodies or functional fragments thereof can bind to the same target (e.g., an EPO protein, an EPO receptor subunit, or a CD131 subunit, etc.), wherein each of the at least two of the one, two, three, four, five, six, seven, eight, nine, ten, or more different antibodies or functional fragments thereof can bind to a different part of the same target, and at least two of the one, two, three, four, five, six, seven, eight, nine, ten, or more different antibodies or functional fragments thereof can bind to a different target (e.g., an EPO protein, an EPO receptor subunit, a CD131 subunit, or a combination thereof).

### Modifications of Antibodies

Antibodies described herein can have one or more modifications that can enhance their activity, binding, specificity, selectivity, or another feature. In some aspects, an anti-EPOR antibody, and/or an anti-CD131 antibody, and/or an anti-EPO antibody, and/or an engineered EPO can include a moiety that extends a half-life (T_{1/2}) or/and the duration of action of the antibody. In some embodiments, the moiety can extend the circulation T_{1/2}, blood T_{1/2}, plasma T_{1/2}, serum T_{1/2}, terminal T_{1/2}, biological T_{1/2}, elimination T_{1/2} or functional T_{1/2}, or any combination thereof, of the antibody. In some embodiments, an Fc portion of an antibody described herein can be modified to extend half-life of the antibody.

In one aspect, an anti-EPOR antibody and/or anti-CD131 antibody and/or an anti-EPO antibody and/or an engineered EPO may be modified by a single moiety. In another aspect, an anti-EPOR antibody and/or an anti-CD131 antibody and/or an anti-EPO antibody and/or an engineered EPO may be modified by two or more substantially similar or identical moieties or two or more moieties of the same type. In some embodiments, an anti-EPOR antibody and/or an anti-CD131 antibody and/or an anti-EPO antibody and/or an engineered EPO may include two or more moieties of different types, or two or more different types of moieties. In some embodiments, two or more anti-EPOR antibodies and/or anti-CD131 antibodies and/or anti-EPO antibodies and/or engineered EPOs can also be attached to one moiety. In some embodiments, the attachment between the anti-EPOR antibody and/or anti-CD131 antibody and/or anti-EPO antibody and/or engineered EPO and the moiety can be covalent or noncovalent.

In some aspects, a polypeptide moiety can be recombinantly fused to the N-terminus or the C-terminus of the heavy chain or the light chain of an anti-EPOR antibody and/or an anti-CD131 antibody and/or an anti-EPO antibody and/or an engineered EPO, optionally via a linker. In some embodiments, the linker may comprise about 4-30 amino acid residues. For example, the linker may comprise from about 6 or 8 amino acid residues to about 20 amino acid residues, or from about 6 or 8 amino acid residues to about 15 amino acid residues.

In some aspects, a protracting moiety can be human serum albumin (HSA) or a portion thereof (e.g., domain III) that binds to the neonatal Fc receptor (FcRn). The HSA or FcRn-binding portion thereof can optionally have one or more mutations that confer a beneficial property or effect. In some embodiments, the HSA or FcRn-binding portion thereof can comprise one or more mutations that can enhance pH-dependent HSA binding to FcRn or/and increase HSA half-life, such as K573P or/and E505G/V547A. In some embodiments, a protracting moiety can be an unstructured polypeptide.

In some aspects, a protracting moiety can be a carboxy-terminal peptide (CTP) derived from the β-subunit of human chorionic gonadotropin (hCG). In the human body, the fourth, fifth, seventh and eight serine residues of the 34-aa CTP of hCG-β typically are attached to O-glycans terminating with a sialic acid residue.

In some aspects, a protracting moiety can be 1, 2, 3, 4, 5, or more moieties of a synthetic polymer. In some embodiments, the synthetic polymer can be biodegradable or non-biodegradable. Biodegradable polymers useful as protracting moieties can include, but are not limited to, poly(2-methacryloyloxyethyl phosphorylcholine) (PMPC) and poly[oligo(ethylene glycol) methyl ether methacrylate] (POEGMA). Non-biodegradable polymers useful as protracting moieties include without limitation poly(ethylene glycol) (PEG), polyglycerol, poly(N-(2-hydroxypropyl) methacrylamide) (PHPMA), polyoxazolines and poly(N-vinylpyrrolidone) (PVP). In some embodiments, a synthetic polymer can be polyethylene glycol (PEG). PEGylation can be done by chemical or enzymatic, site-specific coupling or by random coupling.

In some embodiments, the individual mass (e.g., average molecular weight), or the total mass, of the one or more synthetic polymer moieties can be about 10-50 kDa, about 10-20 kDa, about 20-30 kDa, about 30-40 kDa, or kDa 40-50 kDa. In some embodiments, the individual mass (e.g., average molecular weight), or the total mass, of the one or more synthetic polymer moieties can be about 10 kDa, about 20 kDa, about 30 kDa, about 40 kDa, or 50 kDa. In some embodiments, the individual mass (e.g., average MW), or the total mass, of the one or more synthetic polymer moieties can be greater than about 50 kDa, such as about 50-100 kDa, about 50-60 kDa, about 60-70 kDa, about 70-80 kDa, about 80-90 kDa, or about 90-100 kDa. In some embodiments, the individual mass (e.g., average molecular weight), or the total mass, of the one or more synthetic polymer moieties can be about 60 kDa, about 70 kDa, about 80 kDa, about 90 kDa, or about 100 kDa. In some embodiments, the mass (e.g., average MW) of an individual synthetic polymer moiety can be less than about 10 kDa, such as about 1-5 kDa, about 5-10 kDa, or about 5 kDa. In some embodiments, the individual mass (e.g., average MW), or the total mass, of the one or more synthetic polymer (e.g., PEG) moieties can be about 20 kDa or about 40 kDa.

In some aspects, modified antibodies can comprise a human modified antibody. In some aspects, also provided herein are amino acid sequence variants of modified antibodies which can be prepared by introducing appropriate nucleotide changes into the DNA sequence of modified antibodies, or by synthesis of the desired modified antibody polypeptides. In some embodiments, such variants can include, for example, a deletion, an insertion, or a substitution of one or more residues within the amino acid sequence of an antibody. In some embodiments, any combinations of deletion, insertion, and substitution can be made to generate an antibody that can have desired antigen-binding characteristics. The amino acid changes of a modified antibody can also alter post-translational processes of the modified antibody, including, but are not limited to, changing the number or position of glycosylation sites. In some embodiments, alanine scanning mutagenesis can be used to identify one or more residues or regions of a modified antibody that may be preferred locations for mutagenesis. In some embodiments, a residue or a group of target residues can be identified (e.g., charged residues such as Arg, Asp, His, Lys, and Glu) and replaced by a neutral or negatively charged amino acid (e.g., alanine or polyalanine) to affect an interaction of the amino acids with the surrounding aqueous environment in or outside a cell. In some embodiments, one or more domains demonstrating functional sensitivity to amino acid substitutions can be refined by introducing further amino acid substitution or other substitutions. In some embodiments, amino acid substitutions can include one or more conservative amino acid replacements in non-functional regions of a modified antibody.

In some embodiments, anti-EPO antibodies, anti-EPOR antibodies, or anti-CD131 antibodies described herein can comprise an amino acid sequence with one or more insertions, deletions, and/or substitutions relative to any one of SEQ ID NOs: 1-4, 147-1086, 3805-3814, 3824-4403, or 5616-5991. For example, anti-EPO antibodies, anti-EPOR antibodies, or anti-CD131 antibodies described herein can comprise an amino acid sequence with at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, or at least 30 amino acid insertions, deletions, and/or substitutions relative to any one of SEQ ID NOs: 1-4, 147-1086, 3805-3814, 3824-4403, or 5616-5991. In some embodiments, anti-EPO antibodies, anti-EPOR antibodies, or anti-CD131 antibodies described herein can comprise an amino acid sequence with at most 1, at most 2, at most 3, at most 4, at most 5, at most 6, at most 7, at most 8, at most 9, at most 10, at most 11, at most 12, at most 13, at most 14, at most 15, at most 16, at most 17, at most 18, at most 19, at most 20, at most 25, at most 30, at most 35, at most 40, at most 45, or at most 50 amino acid insertions, deletions, and/or substitutions relative to any one of SEQ ID NOs: 1-4, 147-1086, 3805-3814, 3824-4403, or 5616-5991. In some embodiments, anti-EPO antibodies, anti-EPOR antibodies, or anti-CD131 antibodies described herein can comprise 1, 2, 3, 4, 5, 6, 7, 8, 9, 10, 11, 12, 13, 14, 15, 16, 17, 18, 19, 20, 25, 30, 35, 40, 45, or 50 amino acid insertions, deletions, and/or substitutions relative to any one of SEQ ID NOs: 1-4, 147-1086, 3805-3814, 3824-4403, or 5616-5991. In some embodiments, the one or more insertions, deletions, and/or substitutions can be at the N-terminus, the C-terminus, within the amino acid sequence, or a combination thereof. In some embodiments, the one or more insertions, deletions, and/or substitutions can be contiguous, non-contiguous, or a combination thereof.

In some embodiments, the one or more amino acid substitutions described herein can comprise a conservative amino acid substitution. For example, anti-EPO antibodies, anti-EPOR antibodies, or anti-CD131 antibodies described herein can comprise one or more conservative amino acid substitutions. In some embodiments, anti-EPO antibodies, anti-EPOR antibodies, or anti-CD131 antibodies described herein can comprise an amino acid sequence with at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, or at least 30 conservative amino acid substitutions relative to any one of SEQ ID NOs: 1-4, 147-1086, 3805-3814, 3824-4403, or 5616-5991. In some embodiments, anti-EPO antibodies, anti-EPOR antibodies, or anti-CD131 antibodies described herein can comprise an amino acid sequence with at most 1, at most 2, at most 3, at most 4, at most 5, at most 6, at most 7, at most 8, at most 9, at most 10, at most 11, at most 12, at most 13, at most 14, at most 15, at most 16, at most 17, at most 18, at most 19, at most 20, at most 25, at most 30, at most 35, at most 40, at most 45, or at most 50 conservative amino acid substitutions relative to any one of SEQ ID NOs: 1-4, 147-1086, 3805-3814, 3824-4403, or 5616-5991. In some embodiments, anti-EPO antibodies, anti-EPOR antibodies, or anti-CD131 antibodies described herein can comprise 1, 2, 3, 4, 5, 6, 7, 8, 9, 10, 11, 12, 13, 14, 15, 16, 17, 18, 19, 20, 25, 30, 35, 40, 45, or 50 conservative amino acid substitutions relative to any one of SEQ ID NOs: 1-4, 147-1086, 3805-3814, 3824-4403, or 5616-5991.

In some aspects, modifications of antibodies described herein can be covalent modifications. In some embodiments, covalent modifications can be introduced by reacting one or more targeted amino acid residues of an antibody or functional fragment thereof with an organic derivatizing agent that can be capable of reacting with selected side chains or the N- or C-terminal residues. In some embodiments, covalent modifications can be introduced by altering the native glycosylation pattern of an antibody. For example, one or more carbohydrate moieties can be deleted from an antibody. For example, one or more glycosylation sites that are not present in an antibody can be added. In some embodiments, addition of glycosylation sites to an antibody can be accomplished by altering the amino acid sequence such that it contains one or more N-linked glycosylation sites. In some embodiments, addition of glycosylation sites to an antibody can be accomplished by adding or substituting one or more serine or threonine residues of an antibody (for O-linked glycosylation sites). In some embodiments, a number of carbohydrate moieties on an antibody can be increased by chemical or enzymatic coupling of glycosides to the antibody. In some embodiments, carbohydrate moieties present on an antibody can be removed chemically or enzymatically. In some embodiments, one or more of non-proteinaceous polymers (e.g., polyethylene glycol, polypropylene glycol, or polyoxyalkylenes) can be covalently added to an antibody.

In some embodiments, antibodies described herein can be attached at their C-terminal end to all, or part, of an immunoglobulin heavy chain derived from any antibody isotype, e.g., IgG, IgA, IgE, IgD, or IgM, or any of the isotype sub-classes, e.g., IgG1, IgG2b, IgG2a, IgG3, or IgG4. In some embodiments, antibodies, or functional fragments thereof may be glycosylated. In some embodiments, glycosylation at a variable domain framework residue can alter the binding interaction of the antibody with antigen. In some embodiments, antibodies, or functional fragments thereof may be modified by adding polyethylene glycol (PEG). In some embodiments, addition of PEG can lead to one or more of improved circulation time, improved solubility, improved resistance to proteolysis, reduced antigenicity and immunogenicity, improved bioavailability, reduced toxicity, improved stability, and/or easier formulation. In some embodiments, antibodies, or functional fragments thereof can be conjugated to, or recombinantly engineered with, an affinity tag (e.g., a purification tag).

### Engineered EPO Receptor Fusion Molecules or EPO Traps

The disclosure also encompasses engineered EPORs comprising extracellular domain (ECD) of EPOR. The ECD of EPOR comprises 2 domains, D1 and D2, and these two domains are required for EPO binding. In some embodiments, Fc fusion protein of ECD EPOR-Fc can bind to EPO. In some embodiments, Fc fusion protein of ECD EPOR-Fc can block EPOR activation. In some embodiments, Fc fusion protein of ECD EPOR-Fc can comprise a mutation. For example, Fc fusion protein of ECD EPOR-Fc can comprise a mutation at amino acid residue F93. In some embodiments, Fc fusion protein of ECD EPOR-Fc can comprise F93A mutation. In some embodiments, Fc fusion protein of ECD EPOR-Fc comprising F93A mutation may not bind EPO. For example, a monomeric EPOR ECD comprising F93A mutation or a dimeric EPOR-Fc comprising F93A mutation may not bind EPO.

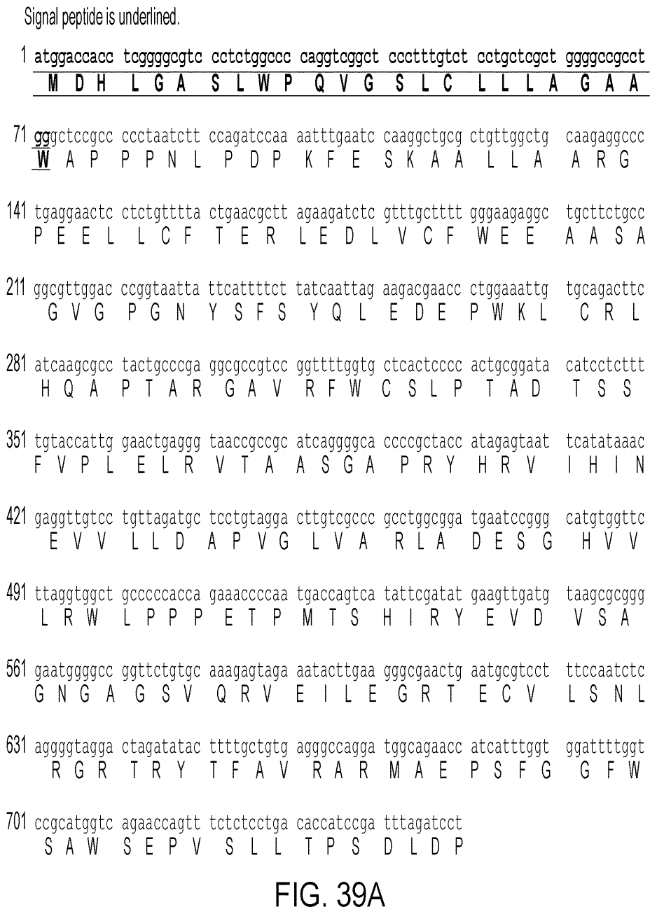

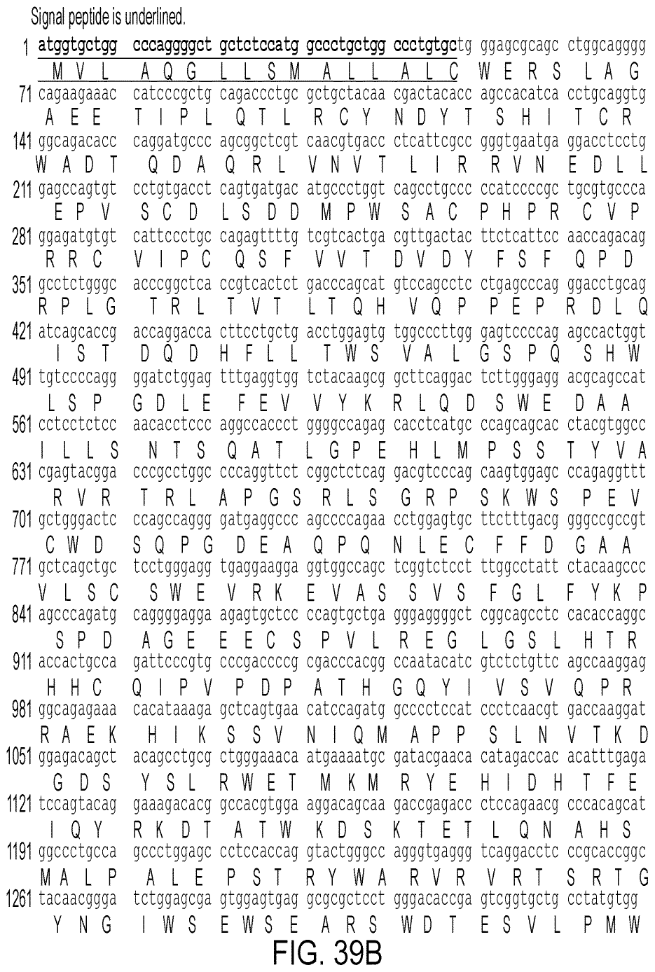

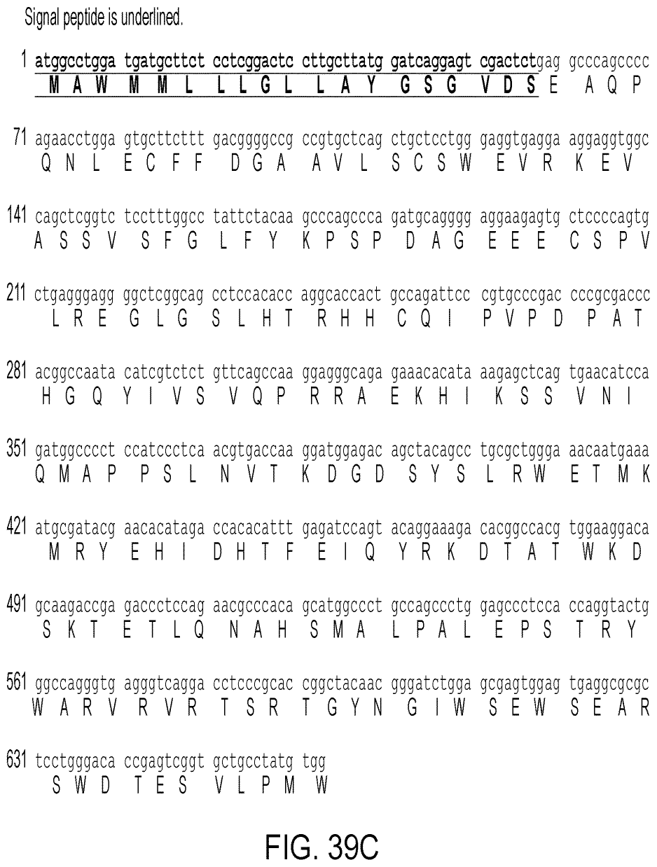

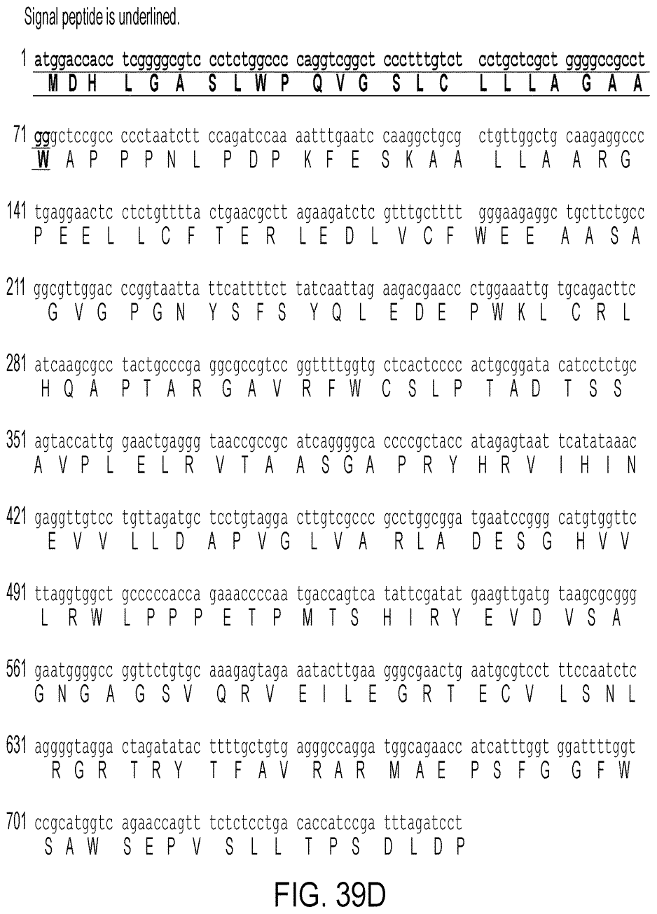

The disclosure also encompasses engineered hetero-EPORs comprising extra cellular domain (ECD) of CD131. The ECD of CD131 comprises 4 domains, D1, D2, D3, and D4. D1 and D2 domains are responsible for dimerization distal to the cell membrane. Without wishing to be bound by theory, D3 and D4 domains can be the regions interacting with EPOR to form a hetero-EPOR. In some embodiments, knobs-in-holes technology can be used to generate heterodimeric Fc fusion proteins with EPOR ECD and CD131 ECD. Non-limiting examples of designs of heterodimeric Fc fusion proteins with EPOR ECD and CD131 ECD are shown in Table 1-2 and the sequences are shown in FIGS. 39A-39D. In some embodiments, EPO binding may require D3 and D4 domains of CD131. For example, the monomeric or dimeric EPOR with the F93A substitution may not bind EPO, however, a hetero-EPOR of a monomeric EPOR with the F93A mutation and a CD131 monomer binds EPO. It seems that EPO binding to the hetero-EPOR is specific to CDC131 subunit. In some embodiments, heterodimeric EPOR (F93A)/CD131-Fc may be used to specifically block hetero-EPORs but not homo-EPORs. In some embodiments, the EPOR ECD and CD131 ECD lack the signal sequence of the polypeptide.

TABLE 1-2
Design of Heterodimeric EPOR/CD131-Fc Fusion Proteins

| EPOR Arm (holes) | CD131 Arm (knobs) |
|---|---|
| hEPOR ECD | |
| | hCD131 ECD |
| | hCD131 D3D4 |
| | hCD131 D4 |
| hEPOR ECD (F93A) | |

### RNAi and Small Molecules

RNAi and small molecules that reduce expression or activity of EPO, EPOR, and/or CD131 can be used to overcome tumor suppressive microenvironments in certain tumors. RNAi includes, for example, siRNA, miRNA, antisense RNA, lncRNA, etc.

RNA interference is a method of post-transcriptional gene regulation that is conserved throughout many eukaryotic organisms. RNAi can be induced by short (i.e., <30 nucleotide) double stranded RNA (“dsRNA”) molecules which are present in the cell. These short dsRNA molecules, called “short interfering RNA” or “siRNA,” cause the destruction of target RNAs which share sequence homology with the siRNA. It is believed that the siRNA and the targeted RNA bind to an “RNA-induced silencing complex” or “RISC,” which cleaves the targeted RNA. The siRNA can be recycled much like a multiple-turnover enzyme, with a single siRNA molecule capable of inducing cleavage of approximately 1000 target RNA molecules.

In an aspect, the disclosure relates to regulatory RNAs for inhibiting the expression of EPO (erythropoietin), EPOR (erythropoietin receptor) and/or CD131.

Regulatory RNAs (e.g., siRNAs) described herein can target EPO mRNA to reduce the half-life and/or function of the EPO mRNA. Regulatory RNAs (e.g., siRNAs) can target exons and UTRs of the EPO mRNA. The cDNA sequence of human EPO (NCBI Reference Sequence: NM_000799.4) is:

| | (SEQ ID NO: 5) |
|---|---|
| 1 | cctttcccag atagcacgct ccgccagtcc caagggtgcg caaccggctg cactcccctc |
| 61 | ccgcgaccca gggcccggga gcagccccca tgacccacac gcacgtctgc agcagccccg |
| 121 | ctcacgcccc ggcgagcctc aacccaggcg tcctgcccct gctctgaccc cgggTggccc |
| 181 | ctacccctgg cgacccctca cgcacacagc ctctccccca cccccacccg cgcacgcaca |
| 241 | catgcagata acagccccga cccccggcca gagccgcaga gtccctgggc caccccggcc |
| 301 | gctcgctgcg ctgcgccgca ccgcgctgtc ctcccggagc cggaccgggg ccaccgcgcc |
| 361 | cgctctgctc cgacaccgcg ccccctggac agccgccctc tcctccaggc ccgtggggct |
| 421 | ggccctgcac cgccgagctt cccgggatga gggcccccgg tgtggtcacc cggcgcgccc |
| 481 | caggtcgctg agggaccccg gccaggcgcg gagatggggg tgcacgaatg tcctgcctgg |
| 541 | ctgtggcttc tcctgtccct gctgtcgctc cctctgggcc tcccagtcct gggcgcccca |
| 601 | ccacgcctca tctgtgacag ccgagtcctg gagaggtacc tcttggaggc caaggaggcc |
| 661 | gagaatatca cgacgggctg tgctgaacac tgcagcttga atgagaatat cactgtccca |
| 721 | gacaccaaag ttaatttcta tgcctggaag aggatggagg tcgggcagca ggccgtagaa |
| 781 | gtctggcagg gcctggccct gctgtcggaa gctgtcctgc ggggccaggc cctgttggtc |
| 841 | aactcttccc agccgtggga gcccctgcag ctgcatgtgg ataaagccgt cagtggcctt |
| 901 | cgcagcctca ccactctgct tcgggctctg ggagcccaga aggaagccat ctcccctcca |
| 961 | gatgcggcct cagctgctcc actccgaaca atcactgctg acactttccg caaactcttc |
| 1021 | cgagtctact ccaatttcct ccggggaaag ctgaagctgt acacagggga ggcctgcagg |
| 1081 | acaggggaca gatgaccagg tgtgtccacc tgggcatatc caccacctcc ctcaccaaca |
| 1141 | ttgcttgtgc cacaccctcc cccgccactc ctgaaccccg tcgaggggct ctcagctcag |
| 1201 | cgccagcctg tcccatggac actccagtgc cagcaatgac atctcagggg ccagaggaac |
| 1261 | tgtccagaga gcaactctga gatctaagga tgtcacaggg ccaacttgag ggcccagagc |
| 1321 | aggaagcatt cagagagcag ctttaaactc agggacagag ccatgctggg aagacgcctg |
| 1381 | agctcactcg gcaccctgca aaatttgatg ccaggacacg ctttggaggc gatttacctg |
| 1441 | ttttcgcacc taccatcagg gacaggatga cctggataac ttaggtggca agctgtgact |
| 1501 | tctccaggtc tcacgggcat gggcactccc ttggtggcaa gagccccctt gacaccgggg |
| 1561 | tggtgggaac catgaagaca ggatgggggc tggcctctgg ctctcatggg gtccaagttt |
| 1621 | tgtgtattct tcaacctcat tgacaagaac tgaaaccacc aa. |

Exemplary nucleic acids encoding RNAi targeting mRNA encoding EPO include siRNA targeting the sequences (these sequences will have U instead of T in the mRNA):

(SEQ ID NO: 6)
CTTGAATGAGAATATCACTGTCCCA
(SEQ ID NO: 7)
GCAGCTTGAATGAGAATATCACTGT
(SEQ ID NO: 8)
GCATGTGGATAAAGCCGTCAGTGGC
(SEQ ID NO: 9)
CCGAACAATCACTGCTGACACTTTC
(SEQ ID NO: 10)
CTTTCCGCAAACTCTTCCGAGTCTA
(SEQ ID NO: 11)
AAACTCTTCCGAGTCTACTCCAATT
(SEQ ID NO: 12)
GAGAGCAACTCTGAGATCTAAGGAT
(SEQ ID NO: 13)
AGAGCAACTCTGAGATCTAAGGATG
(SEQ ID NO: 14)
GAGCAACTCTGAGATCTAAGGATGT
(SEQ ID NO: 15)
CAGGAAGCATTCAGAGAGCAGCTTT
(SEQ ID NO: 16)
AGGAAGCATTCAGAGAGCAGCTTTA
(SEQ ID NO: 17)
GAAGCATTCAGAGAGCAGCTTTAAA
(SEQ ID NO: 18)
GAGAGCAGCTTTAAACTCAGGGACA
(SEQ ID NO: 19)
CAGGACACGCTTTGGAGGCGATTTA
(SEQ ID NO: 20)
CATCAGGGACAGGATGACCTGGATA
(SEQ ID NO: 21)
GGGACAGGATGACCTGGATAACTTA

Regulatory RNAs (e.g., siRNAs) described herein can target EPOR mRNA to reduce the half-life and/or function of the EPOR mRNA. Regulatory RNAs (e.g., siRNAs) can target exons and UTRs of the EPOR mRNA. The cDNA sequence of human EPOR (NCBI Reference Sequence: NM_000121.4) is:

| (SEQ ID NO: 22) | |
|---|---|
| 1 | ggtcagctgc gtccggcgga ggcagctgct gacccagctg tggactgtgc cgggggcggg |
| 61 | ggacggaggg gcaggagccc tgggctcccc gtggcggggg ctgtatcatg gaccacctcg |
| 121 | gggcgtccct ctggccccag gtcggctccc tttgtctcct gctcgctggg gccgcctggg |
| 181 | cgcccccgcc taacctcccg gaccccaagt tcgagagcaa agcggccttg ctggcggccc |
| 241 | gggggcccga agagcttctg tgcttcaccg agcggttgga ggacttggtg tgtttctggg |
| 301 | aggaagcggc gagcgctggg gtgggcccgg gcaactacag cttctcctac cagctcgagg |
| 361 | atgagccatg gaagctgtgt cgcctgcacc aggctcccac ggctcgtggt gcggtgcgct |
| 421 | tctggtgttc gctgcctaca gccgacacgt cgagcttcgt gcccctagag ttgcgcgtca |
| 481 | cagcagcctc cggcgctccg cgatatcacc gtgtcatcca catcaatgaa gtagtgctcc |
| 541 | tagacgcccc cgtggggctg gtggcgcggt tggctgacga gagcggccac gtagtgttgc |
| 601 | gctggctccc gccgcctgag acacccatga cgtctcacat ccgctacgag gtggacgtct |
| 661 | cggccggcaa cggcgcaggg agcgtacaga gggtggagat cctggagggc cgcaccgagt |
| 721 | gtgtgctgag caacctgcgg ggccggacgc gctacacctt cgccgtccgc gcgcgtatgg |
| 781 | ctgagccgag cttcggcggc ttctggagcg cctggtcgga gcctgtgtcg ctgctgacgc |
| 841 | ctagcgacct ggaccccctc atcctgacgc tctccctcat cctcgtggtc atcctggtgc |
| 901 | tgctgaccgt gctcgcgctg ctctcccacc gccgggctct gaagcagaag atctggcctg |
| 961 | gcatcccgag cccagagagc gagtttgaag gcctcttcac cacccacaag ggtaacttcc |
| 1021 | agctgtggct gtaccagaat gatggctgcc tgtggtggag cccctgcacc cccttcacgg |
| 1081 | aggacccacc tgcttccctg gaagtcctct cagagcgctg ctgggggacg atgcaggcag |
| 1141 | tggagccggg gacagatgat gagggccccc tgctggagcc agtgggcagt gagcatgccc |
| 1201 | aggataccta tctggtgctg gacaaatggt tgctgccccg gaacccgccc agtgaggacc |
| 1261 | tcccagggcc tggtggcagt gtggacatag tggccatgga tgaaggctca gaagcatcct |
| 1321 | cctgctcatc tgctttggcc tcgaagccca gcccagaggg agcctctgct gccagctttg |
| 1381 | agtacactat cctggacccc agctcccagc tcttgcgtcc atggacactg tgccctgagc |
| 1441 | tgccccctac cccaccccac ctaaagtacc tgtaccttgt ggtatctgac tctggcatct |
| 1501 | caactgacta cagctcaggg gactcccagg gagcccaagg gggcttatcc gatggcccct |
| 1561 | actccaaccc ttatgagaac agccttatcc cagccgctga gcctctgccc cccagctatg |
| 1621 | tggcttgctc ttaggacacc aggctgcaga tgatcaggga tccaatatga ctcagagaac |
| 1681 | cagtgcagac tcaagactta tggaacaggg atggcgaggc ctctctcagg agcaggggca |
| 1741 | ttgctgattt tgtctgccca atccatcctg ctcaggaaac cacaaccttg cagtattttt |
| 1801 | aaatatgtat agtttttttt ttatctata tatatatata cacatatgta tgtaagtttt |
| 1861 | tctaccatga tttctacaaa caccctttaa gtcccatctt cccctgggca taggccatag |
| 1921 | ggatagaagt taaagttctt gagcttattc agaagctgga tctgcaatct gaatgctact |
| 1981 | cataacataa caaaatagta tgttaaacag ctcttaaatc ttactggctt accacattaa |
| 2041 | atgatttctc tctcctaact cagctcaaat gggcagccat ccatgggatg agtcagaggt |
| 2101 | tcagactctt ccagtctgta gctctacctt ctcttagggt acttagatgg atcccctgtt |
| 2161 | ctacaaactg ccagtcagca agggaagaaa aagggcagca atgaccctca atgggccatt |
| 2221 | tgagggatct ggcctggaaa tgggcttcct ctcttcttct cacacctcac tggctggaaa |
| 2281 | cagtcacatg accccagtca catgaaaggc caggaaactt agtttagctg tacacccagg |
| 2341 | aagggcaaag ctgtttaagg gccactagct agtctctgcc actaataata ataaaagtaa |
| 2401 | ttctgaatca g. |

Exemplary nucleic acids encoding RNAi targeting mRNA encoding EPOR include siRNA targeted at the following sequences (these sequence will be in the mRNA with U instead of T):

(SEQ ID NO: 23)
CACCGAGCGGTTGGAGGACTTGGTG
(SEQ ID NO: 24)
CGAGGATGAGCCATGGAAGCTGTGT
(SEQ ID NO: 25)
ATGGAAGCTGTGTCGCCTGCACCAG
(SEQ ID NO: 26)
CACCAGGCTCCCACGGCTCGTGGTG
(SEQ ID NO: 27)
ATATCACCGTGTCATCCACATCAAT
(SEQ ID NO: 28)
ACATCAATGAAGTAGTGCTCCTAGA
(SEQ ID NO: 29)
ATCAATGAAGTAGTGCTCCTAGACG
(SEQ ID NO: 30)
CGTGGGGCTGGTGGCGCGGTTGGCT
(SEQ ID NO: 31)
CTGGAGGGCCGCACCGAGTGTGTGC
(SEQ ID NO: 32)
ACCACCCACAAGGGTAACTTCCAGC
(SEQ ID NO: 33)
CAGAATGATGGCTGCCTGTGGTGGA
(SEQ ID NO: 34)
AGCGCTGCTGGGGGACGATGCAGGC
(SEQ ID NO: 35)
GAGGGAGCCTCTGCTGCCAGCTTTG
(SEQ ID NO: 36)
CCTGTACCTTGTGGTATCTGACTCT
(SEQ ID NO: 37)
ATCTGACTCTGGCATCTCAACTGAC
(SEQ ID NO: 38)
TCTGGCATCTCAACTGACTACAGCT
(SEQ ID NO: 39)
CAGGGGACTCCCAGGGAGCCCAAGG
(SEQ ID NO: 40)
AGCCTCTGCCCCCCAGCTATGTGGC
(SEQ ID NO: 41)
CTCAAGACTTATGGAACAGGGATGG
(SEQ ID NO: 42)
CTTACTGGCTTACCACATTAAATGA

Regulatory RNAs (e.g., siRNAs) described herein can target CD131 mRNA to reduce the half-life and/or function of the CD131 mRNA. Regulatory RNAs (e.g., siRNAs) can target exons and UTRs of the CD131 mRNA. The cDNA sequence of CD131 (NCBI Reference Sequence: NM_000395.3) is:

| (SEQ ID NO: 43) | |
|---|---|
| 1 | actctgccta gaggctccag aagaagactg gtctctccca ccacacagag gcctggagga |
| 61 | ggcagaggcc aggagggaga ggtcccaaga gcctgtgaaa tgggtctggc ctggctccca |
| 121 | gctgggcagg aacacaggac ttcaggacac taaggaccct gtcatgccca tggccagcac |
| 181 | ccaccagtgc tggtgcctgc ctgtccagag ctgaccaggg agatggtgct ggcccagggg |
| 241 | ctgctctcca tggccctgct ggccctgtgc tgggagcgca gcctggcagg ggcagaagaa |
| 301 | accatcccgc tgcagaccct gcgctgctac aacgactaca ccagccacat cacctgcagg |
| 361 | tgggcagaca cccaggatgc ccagcggctc gtcaacgtga ccctcattcg ccgggtgaat |
| 421 | gaggacctcc tggagccagt gtcctgtgac ctcagtgatg acatgccctg gtcagcctgc |
| 481 | ccccatcccc gctgcgtgcc caggagatgt gtcattccct gccagagttt tgtcgtcact |
| 541 | gacgttgact acttctcatt ccaaccagac aggcctctgg gcacccggct caccgtcact |
| 601 | ctgacccagc atgtccagcc tcctgagccc agggacctgc agatcagcac cgaccaggac |
| 661 | cacttcctgc tgacctggag tgtggccctt gggagtcccc agagccactg gttgtcccca |
| 721 | ggggatctgg agtttgaggt ggtctacaag cggcttcagg actcttggga ggacgcagcc |
| 781 | atcctcctct ccaacacctc ccaggccacc ctggggccag agcacctcat gcccagcagc |
| 841 | acctacgtgg cccgagtacg gacccgcctg gccccaggtt ctcggctctc aggacgtccc |
| 901 | agcaagtgga gcccagaggt ttgctgggac tcccagccag gggatgaggc ccagccccag |
| 961 | aacctggagt gcttctttga cggggccgcc gtgctcagct gctcctggga ggtgaggaag |
| 1021 | gaggtggcca gctcggtctc ctttggccta ttctacaagc ccagcccaga tgcaggggag |
| 1081 | gaagagtgct ccccagtgct gagggagggg ctcggcagcc tccacaccag gcaccactgc |
| 1141 | cagattcccg tgcccgaccc cgcgacccac ggccaataca tcgtctctgt tcagccaagg |
| 1201 | agggcagaga aacacataaa gagctcagtg aacatccaga tggcccctcc atccctcaac |
| 1261 | gtgaccaagg atggagacag ctacagcctg cgctgggaaa caatgaaaat gcgatacgaa |
| 1321 | cacatagacc acacatttga gatccagtac aggaaagaca cggccacgtg gaaggacagc |
| 1381 | aagaccgaga ccctccagaa cgcccacagc atggccctgc cagccctgga gccctccacc |
| 1441 | aggtactggg ccagggtgag ggtcaggacc tcccgcaccg gctacaacgg gatctggagc |
| 1501 | gagtggagtg aggcgcgctc ctgggacacc gagtcggtgc tgcctatgtg ggtgctggcc |
| 1561 | ctcatcgtga tcttcctcac catcgctgtg ctcctggccc tccgcttctg tggcatctac |
| 1621 | gggtacaggc tgcgcagaaa gtgggaggag aagatcccca accccagcaa gagccacctg |
| 1681 | ttccagaacg ggagcgcaga gctttggccc ccaggcagca tgtcggcctt cactagcggg |
| 1741 | agtcccccac accaggggcc gtggggcagc cgcttccctg agctggaggg ggtgttccct |
| 1801 | gtaggattcg gggacagcga ggtgtcacct ctcaccatag aggaccccaa gcatgtctgt |
| 1861 | gatccaccat ctgggcctga cacgactcca gctgcctcag atctacccac agagcagccc |
| 1921 | cccagccccc agccaggccc gcctgccgcc tcccacacac ctgagaaaca ggcttccagc |
| 1981 | tttgacttca atgggcccta cctggggccg ccccacagcc gctccctacc tgacatcctg |
| 2041 | ggccagccgg agcccccaca ggagggtggg agccagaagt ccccacctcc agggtccctg |
| 2101 | gagtacctgt gtctgcctgc tggggggcag gtgcaactgg tccctctggc ccaggcgatg |
| 2161 | ggaccaggac aggccgtgga agtggagaga aggccgagcc agggggctgc agggagtccc |
| 2221 | tccctggagt ccgggggagg ccctgcccct cctgctcttg ggccaagggt gggaggacag |
| 2281 | gaccaaaagg acagccctgt ggctataccc atgagctctg gggacactga ggaccctgga |
| 2341 | gtggcctctg gttatgtctc ctctgcagac ctggtattca ccccaaactc aggggcctcg |
| 2401 | tctgtctccc tagttccctc tctgggcctc ccctcagacc agacccccag cttatgtcct |
| 2461 | gggctggcca gtggaccccc tggagcccca ggccctgtga agtcagggtt tgagggctat |
| 2521 | gtggagctcc ctccaattga gggccggtcc cccaggtcac caaggaacaa tcctgtcccc |
| 2581 | cctgaggcca aaagccctgt cctgaaccca ggggaacgcc cggcagatgt gtccccaaca |
| 2641 | tccccacagc ccgagggcct ccttgtcctg cagcaagtgg gcgactattg cttcctcccc |
| 2701 | ggcctggggc ccggccctct ctcgctccgg agtaaacctt cttccccggg acccggtcct |
| 2761 | gagatcaaga acctagacca ggcttttcaa gtcaagaagc ccccaggcca ggctgtgccc |
| 2821 | caggtgcccg tcattcagct cttcaaagcc ctgaagcagc aggactacct gtctctgccc |
| 2881 | ccttgggagg tcaacaagcc tggggaggtg tgttgagacc cccaggccta gacaggcaag |
| 2941 | gggatggaga gggcttgcct tccctcccgc ctgaccttcc tcagtcattt ctgcaaagcc |
| 3001 | aaggggcagc ctcctgtcaa ggtagctaga ggcctgggaa aggagatagc cttgctccgg |
| 3061 | cccccttgac cttcagcaaa tcacttctct ccctgcgctc acacagacac acacacacac |
| 3121 | acgtacatgc acacattttt cctgtcaggt taacttattt gtaggttctg cattattaga |
| 3181 | actttctaga tatactcatt ccatctcccc ctcatttttt taatcaggtt tccttgcttt |
| 3241 | tgccattttt cttccttctt ttttcactga tttattatga gagtggggct gaggtctgag |
| 3301 | ctgagcctta tcagactgag atgcggctgg ttgtgttgag gacttgtgtg ggctgcctgt |
| 3361 | ccccggcagt cgctgatgca catgacatga ttctcatctg ggtgcagagg tgggaggcac |
| 3421 | caggtgggca cccgtggggg ttagggcttg gaagagtggc acaggactgg gcacgctcag |
| 3481 | tgaggctcag ggaattcaga ctagcctcga ttgtcactcc gagaaatggg catggtattg |
| 3541 | ggggtcgggg gggcggtgca agggacgcac atgagagact gtttgggagc ttctggggag |
| 3601 | ccctgctagt tgtctcagtg atgtctgtgg gacctccagt cccttgagac cccacgtcat |
| 3661 | gtagagaagt taacggccca agtggtgggc aggctggcgg gacctgggga acatcaggag |
| 3721 | aggagtccag agcccacgtc tactgcggaa aagtcagggg aaactgccaa acaaaggaaa |
| 3781 | atgccccaaa ggcatatatg ctttagggcc tttggtccaa atggcccggg tggccactct |
| 3841 | tccagataga ccaggcaact ctccctccca ccggccacag atgaggggct gctgatctat |
| 3901 | gcctgggcct gcaccaggga ttatggttct tttaaatctt tgcctttcag atacaggaaa |
| 3961 | aataatggca ttaaattgct ttaatttgca ttattttagt tatccagttt gcacatattt |
| 4021 | ttataggtat cttaggcatc gattggtatt ttttaactgg gccaagccca ttaaggtctt |
| 4081 | tcttctgttg ggtgctatca ttttctgatt aagtcttttt gactattgac atacagtctt |
| 4141 | tcacagatgg tggagtgttt ttcccccaaa tctgttgttt gtcttataat gttgtatatg |
| 4201 | aggttttatg gtgtatgaat atgaatgctt ctgtaatgtc aaacagatcc ctagtaaact |
| 4261 | ccttcttcac ttttactgtc agatttacaa aggtcctccc attgcaaagc agtgtttgtc |
| 4321 | ctaatttata tattgttttt ctagttcatt ttgtgtttcc aacttttcat gtaaaatttt |
| 4381 | aattattttt gaatgtgtgg atgtgagact gaggtgcctt ttggtactga aattcttttt |
| 4441 | ccatgtacct gaagtgttac ttttgtgata taggaaatcc ttgtatatat actttattgg |
| 4501 | tccctaggct tcctattttg ttaccttgct ttctctatgg catccaccat tttgattgtt |
| 4561 | ctacttttat gatatgtttt cataagtggt taagcaagta ttctcgttac ttttgctctt |
| 4621 | aaatccctat tcattacagc aatgttggtg gtcaaagaaa atgataaaca acttgaatgt |
| 4681 | tcaatggtcc tgaaatacat aacaacattt tagtacattg taaagtagaa tcctctgttc |
| 4741 | ataatgaaca agatgaacca atgtggatta gaaagaagtc cgagatatta attccaaaat |
| 4801 | atccagacat tgttaaaggg aaaaaattgc aataaaatat ttgtaacata aaa. |

Exemplary nucleic acids encoding RNAi targeting mRNA encoding CD131 include siRNA that target the following sequences (these sequences will have U instead of T in the mRNA):

(SEQ ID NO: 44)
GGCTCGTCAACGTGACCCTCATTCG
(SEQ ID NO: 45)
CCTGGAGCCAGTGTCCTGTGACCTC
(SEQ ID NO: 46)
GCCCAGGAGATGTGTCATTCCCTGC
(SEQ ID NO: 47)
GTCGTCACTGACGTTGACTACTTCT
(SEQ ID NO: 48)
GACGTTGACTACTTCTCATTCCAAC
(SEQ ID NO: 49)
CTCCACACCAGGCACCACTGCCAGA
(SEQ ID NO: 50)
ACCCACGGCCAATACATCGTCTCTG
(SEQ ID NO: 51)
ATGGCCCCTCCATCCCTCAACGTGA
(SEQ ID NO: 52)
ACGTGACCAAGGATGGAGACAGCTA
(SEQ ID NO: 53)
GTGACCAAGGATGGAGACAGCTACA
(SEQ ID NO: 54)
AAGGATGGAGACAGCTACAGCCTGC
(SEQ ID NO: 55)
ATGCGATACGAACACATAGACCACA
(SEQ ID NO: 56)
CGATACGAACACATAGACCACACAT
(SEQ ID NO: 57)
GTGGGTGCTGGCCCTCATCGTGATC
(SEQ ID NO: 58)
AGATCCCCAACCCCAGCAAGAGCCA
(SEQ ID NO: 59)
CGGGGACAGCGAGGTGTCACCTCTC
(SEQ ID NO: 60)
CTACCCACAGAGCAGCCCCCCAGCC
(SEQ ID NO: 61)
AAAGGACAGCCCTGTGGCTATACCC
(SEQ ID NO: 62)
CCTGAGATCAAGAACCTAGACCAGG
(SEQ ID NO: 63)
TCAAAGCCCTGAAGCAGCAGGACTA
(SEQ ID NO: 64)
GTCAAAGAAAATGATAAACAACTTG

The regulatory RNA can be complementary to a sequence in the above exons, and can be complementary to about 15 nucleotides to about 30 contiguous nucleotides in the target. The regulatory RNA can have 70%, 75%, 80%, 85%, 90%, 95%, 97%, or 99% sequence identity with the complement to the target sequence. The regulatory RNA can also be one that hybridizes to the target sequence under stringent hybridization conditions. Exemplary regulatory RNAs include, for example a regulatory RNA that is complementary to any 17, 18, 19, 20, 21, 22, 23, 24, 25, 26, 27, 28, 29, 30, 31, or 32 contiguous nucleotides of SEQ ID NO: 5, SEQ ID NO: 22, or SEQ ID NO: 43. Exemplary regulatory RNAs include, for example a regulatory RNA that is complementary to any 21 contiguous nucleotides of SEQ ID NO: 5, SEQ ID NO: 22, or SEQ ID NO: 43.

In an aspect, the disclosure describes isolated siRNA comprising short double-stranded RNA from about 15 nucleotides to about 30 nucleotides in length, or between 18 and 25, or 18, 19, 20, 21, 22, 23, 24, or 25 nucleotides in length and are targeted to the target mRNA. The siRNA comprise a sense RNA strand and a complementary antisense RNA strand annealed together by standard Watson-Crick base-pairing interactions (hereinafter “base-paired”). Each strand of the duplex can be the same length or of different lengths. As is described in more detail below, the sense strand comprises a nucleic acid sequence which is identical to a target sequence contained within the target mRNA. In some cases, the siRNA molecules comprise single-stranded RNAs. In some aspects, the disclosure describes an antisense oligonucleotide (ASO). ASO is an inhibitory polynucleotide that is small (−18-30 nucleotides), synthetic, single-stranded nucleic acid polymers of diverse chemistries, which can be employed to modulate gene expression via various mechanisms. ASOs can be subdivided into two major categories: RNase H competent and steric block. The endogenous RNase H enzyme RNASEH1 recognizes RNA-DNA heteroduplex substrates that are formed when DNA-based oligonucleotides bind to their cognate mRNA transcripts and catalyzes the degradation of RNA. Cleavage at the site of ASO binding results in destruction of the target RNA, thereby silencing target gene expression. This approach has been widely used as a means of downregulating disease-causing or disease-modifying genes.

The sense and antisense strands of an siRNA can comprise two complementary, single-stranded RNA molecules or can comprise a single molecule in which two complementary portions are base-paired and are covalently linked, for example, by a single-stranded hairpin loop. Without wishing to be bound by any theory, it is believed that the hairpin loop of the latter type of siRNA molecule is cleaved intracellularly by the Dicer protein (or its equivalent) to form an siRNA of two individual base-paired RNA molecules.

siRNA can comprise partially purified RNA, substantially pure RNA, synthetic RNA, recombinantly produced RNA, as well as altered RNA that differs from naturally-occurring RNA by the addition, deletion, substitution and/or alteration of one or more nucleotides, or combinations of one or more of the foregoing. Alterations can include addition of non-nucleotide material, such as to the end(s) of the siRNA or to one or more internal nucleotides of the siRNA, including modifications that make the siRNA resistant to nuclease digestion. One or both strands of the siRNA can also comprise a 3′ overhang. As used herein, a 3′ overhang refers to at least one unpaired nucleotide extending from the 3′-end of a duplexed RNA strand. The 3′ overhang can have 1 to about 6 nucleotides (which includes ribonucleotides or deoxynucleotides) in length, or from 1 to about 5 nucleotides in length, or from 1 to about 4 nucleotides in length, or from about 2 to about 4 nucleotides in length. The 3′ overhang can be present on both strands of the siRNA, and can be 2 nucleotides in length. For example, each strand of an siRNA can have 3′ overhangs of dithymidylic acid (TT) or diuridylic acid (UU).

In order to enhance the stability of an siRNA, the 3′ overhangs can be stabilized against degradation. For example, the overhangs can be stabilized by including purine nucleotides, such as adenosine or guanosine nucleotides. The overhangs can also be stabilized by substitution of pyrimidine nucleotides with modified analogues, e.g., substitution of uridine nucleotides in the 3′ overhangs with 2′-deoxythymidine, is tolerated and does not affect the efficiency of RNAi degradation. In particular, the absence of a 2′ hydroxyl in the 2′-deoxythymidine significantly enhances the nuclease resistance of the 3′ overhang in tissue culture medium.

The siRNA can have the sequence AA (N19) TT or NA (N21), where N is any nucleotide. These siRNA can have approximately 30-70% G/C content, and can comprise approximately 50% G/C content. The sequence of the sense siRNA strand can correspond to (N19) TT or N21 (i.e., positions 3 to 23), respectively. In the latter case, the 3′ end of the sense siRNA can be converted to TT. The rationale for this sequence conversion is to generate a symmetric duplex with respect to the sequence composition of the sense and antisense strand 3′ overhangs. The antisense RNA strand can then synthesized as the complement to positions 1 to 21 of the sense strand.

When Position 1 of the 23-nt sense strand is not recognized in a sequence-specific manner by the antisense strand, the 3′-most nucleotide residue of the antisense strand can be chosen deliberately. However, in this case the penultimate nucleotide of the antisense strand (complementary to position 2 of the 23-nt sense strand in either embodiment) is generally complementary to the targeted sequence.

The siRNA can also have the sequence NAR(N17)YNN, where R is a purine (e.g., A or G) and Y is a pyrimidine (e.g., C or U/T). The respective 21-nt sense and antisense RNA strands therefore generally begin with a purine nucleotide. Such siRNA can be expressed from pol III expression vectors without a change in targeting site, as expression of RNAs from pol III promoters is only believed to be efficient when the first transcribed nucleotide is a purine.

The siRNA usually has a sequence having no more than five (5) consecutive purines or pyrimidines. The siRNA also usually comprises a sequence having no more than five (5) consecutive nucleotides having the same nucleobase (i.e., A, C, G, or U/T).

The siRNA can be targeted to any stretch of approximately 19-25 contiguous nucleotides in any of the target mRNA sequences (the “target sequence”). Techniques for selecting target sequences for siRNA are given, for example, in Fakhr et al., Precise and efficient siRNA design: a key point in competent gene silencing, Cancer Gene Therapy 23:73-82 (2016), which is hereby incorporated by reference in its entirety for all purposes. Thus, the sense strand of the present siRNA comprises a nucleotide sequence identical to any contiguous stretch of about 19 to about 25 nucleotides in the target mRNA.

The siRNA can be obtained using a number of techniques known to those of skill in the art. For example, the siRNA can be chemically synthesized or recombinantly produced using methods known in the art, such as the Drosophila in vitro system described in U.S. published application 2002/0086356, which is hereby incorporated by reference in its entirety for all purposes. siRNA can be chemically synthesized using appropriately protected ribonucleoside phosphoramidites and a conventional DNA RNA synthesizer. The siRNA can be synthesized as two separate, complementary RNA molecules, or as a single RNA molecule with two complementary regions. Commercial suppliers of synthetic RNA molecules or synthesis reagents are well known in the art.

siRNA can also be expressed from recombinant circular or linear DNA plasmids using any suitable promoter. Suitable promoters for expressing siRNA from a plasmid include, for example, the U6 or H1 RNA pol III promoter sequences and the cytomegalovirus promoter. Selection of other suitable promoters is within the skill in the art. Recombinant plasmids can also comprise inducible or regulatable promoters for expression of the siRNA in a particular tissue or in a particular intracellular environment.

The siRNA expressed from recombinant plasmids can either be isolated from cultured cell expression systems by standard techniques, or can be expressed intracellularly at or near a target tissue or cells in vivo. siRNA can be expressed from a recombinant plasmid either as two separate, complementary RNA molecules, or as a single RNA molecule with two complementary regions. Selection of plasmids suitable for expressing siRNA, methods for inserting nucleic acid sequences for expressing the siRNA into the plasmid, and methods of delivering the recombinant plasmid to the cells of interest are within the skill in the art. See, for example Tuschl, T. (2002), Nat. Biotechnol, 20:446-448; Brummelkamp T R et al. (2002), Science 296:550-553; Miyagishi M et al. (2002), Nat. Biotechnol. 20:497-500; Paddison P J et al. (2002), Genes Dev. 16:948-958; Lee N S et al. (2002), Nat. Biotechnol. 20:500-505; and Paul C P et al. (2002), Nat. Biotechnol. 20:505-508, all of which are incorporated by reference in their entirety for all purposes.

siRNA can also be expressed from recombinant viral vectors intracellularly at or near the target tissue or cells in vivo. The recombinant viral vectors can comprise sequences encoding the siRNA and any suitable promoter for expressing the siRNA sequences. Suitable promoters include, for example, the U6 or H1 RNA pol III promoter sequences and the cytomegalovirus promoter. Selection of other suitable promoters is within the skill in the art. The recombinant viral vectors can also comprise inducible or regulatable promoters for expression of the siRNA in a particular tissue or in a particular intracellular environment. siRNA can be expressed from a recombinant viral vector either as two separate, complementary RNA molecules, or as a single RNA molecule with two complementary regions.

Any viral vector capable of accepting the coding sequences for the siRNA molecule(s) to be expressed can be used for example vectors derived from adenovirus (AV); adeno-associated virus (AAV); retroviruses (e.g., lentiviruses (LV), Rhabdoviruses, murine leukemia virus); herpes virus, and the like. The tropism of the viral vectors can also be modified by pseudotyping the vectors with envelope proteins or other surface antigens from other viruses. For example, an AAV vector of the invention can be pseudotyped with surface proteins from vesicular stomatitis virus (VSV), rabies, Ebola, Mokola, and the like.

The siRNA can be chemically modified to enhance stability. The siRNA may be synthesized and/or modified by methods well established in the art, such as those described in “Current protocols in nucleic acid chemistry,” Beaucage, S. L. et al. (Eds.), John Wiley & Sons, Inc., New York, N.Y., USA, which is hereby incorporated herein by reference. Specific examples of siRNA compounds include siRNAs containing modified backbones or no natural internucleoside linkages. siRNAs having modified backbones include those that retain a phosphorus atom in the backbone and those that do not have a phosphorus atom in the backbone. Modified siRNAs that do not have a phosphorus atom in their internucleoside backbone can also be considered to be oligonucleosides.

Modified siRNA backbones include, for example, phosphorothioates, chiral phosphorothioates, phosphorodithioates, phosphotriesters, aminoalkylphosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those) having inverted polarity wherein the adjacent pairs of nucleoside units are linked 3′-5′ to 5′-3′ or 2′-5′ to 5′-2′. Various salts, mixed salts and free acid forms are also included.

Representative U.S. patents that teach the preparation of the above phosphorus-containing linkages include, but are not limited to, U.S. Pat. Nos. 3,687,808; 4,469,863; 4,476,301; 5,023,243; 5,177,195; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,316; 5,550,111; 5,563,253; 5,571,799; 5,587,361; and 5,625,050, each of which is incorporated by reference in its entirety for all purposes.

Modified siRNA backbones that do not include a phosphorus atom therein have backbones that are formed by short chain alkyl or cycloalkyl internucleoside linkages, mixed heteroatoms and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH2 component parts.

Representative U.S. patents that teach the preparation of the above oligonucleosides include, but are not limited to, U.S. Pat. Nos. 5,034,506; 5,166,315; 5,185,444; 5,214,134; 5,216,141; 5,235,033; 5,64,562; 5,264,564; 5,405,938; 5,434,257; 5,466,677; 5,470,967; 5,489,677; 5,541,307; 5,561,225; 5,596,086; 5,602,240; 5,608,046; 5,610,289; 5,618,704; 5,623,070; 5,663,312; 5,633,360; 5,677,437; and, 5,677,439, each of which is herein incorporated by reference in its entirety for all purposes.

In other suitable siRNA mimetics, both the sugar and the internucleoside linkage, i.e., the backbone, of the nucleotide units are replaced with novel groups. The base units are maintained for hybridization with an appropriate nucleic acid target compound. One such oligomeric compound, a dsRNA mimetic that has been shown to have excellent hybridization properties, is referred to as a peptide nucleic acid (PNA). In PNA compounds, the sugar backbone of an siRNA is replaced with an amide containing backbone, in particular an aminoethylglycine backbone. The nucleobases are retained and are bound directly or indirectly to aza nitrogen atoms of the amide portion of the backbone. Representative U.S. patents that teach the preparation of PNA compounds include, but are not limited to, U.S. Pat. Nos. 5,539,082; 5,714,331; and 5,719,262, each of which is herein incorporated by reference in its entirety for all purposes. Further teaching of PNA compounds can be found in Nielsen et al., Science, 1991, 254, 1497-1500, which is incorporated by reference in its entirety for all purposes.

In another aspect, siRNAs can have phosphorothioate backbones and oligonucleosides with heteroatom backbones, and in particular —CH₂—NH—CH₂—, —CH₂—N(CH₃)—O—CH₂—[known as a methylene (methylimino) or MMI backbone], —CH₂—O—N(CH₃)—CH₂—, —CH₂—N(CH₃)—N(CH₃)—CH₂—and —N(CH₃)—CH₂—CH₂—[wherein the native phosphodiester backbone is represented as —O—P—O—CH₂—] of the above-referenced U.S. Pat. No. 5,489,677, and the amide backbones of the above-referenced U.S. Pat. No. 5,602,240. Also preferred are dsRNAs having morpholino backbone structures of the above-referenced U.S. Pat. No. 5,034,506.

Modified siRNAs may also contain one or more substituted sugar moieties. siRNAs can comprise one of the following at the 2′ position: OH; F; O—, S—, or N-alkyl; O—, S—, or N-alkenyl; O—, S- or N-alkynyl; or O-alkyl-O-alkyl, wherein the alkyl, alkenyl and alkynyl may be substituted or unsubstituted C₁ to C₁₀ alkyl or C₂ to C₁₀ alkenyl and alkynyl. Particularly preferred are O[(CH₂)ₙO]ₘCH₃, O(CH₂)ₙOCH₃, O(CH₂)ₙNH₂, O(CH₂)ₙCH₃, O(CH₂)ₙONH₂, and O(CH₂)ₙON[(CH₂)ₙCH₃)]₂, where n and m are from 1 to about 10. Other preferred dsRNAs comprise one of the following at the 2′ position: C₁ to C₁₀ lower alkyl, substituted lower alkyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an dsRNA, or a group for improving the pharmacodynamic properties of an dsRNA, and other substituents having similar properties. A preferred modification includes 2′-methoxyethoxy (2′-O—CH₂CH₂OCH₃, also known as 2′-O-(2-methoxyethyl) or 2′-MOE) (Martin et al., Helv. Chim. Acta, 1995, 78, 486-504) i.e., an alkoxy-alkoxy group. A further preferred modification includes 2′-dimethylaminooxyethoxy, i.e., a O(CH₂)₂ON(CH₃)₂ group, also known as 2′-DMAOE, as described in examples herein below, and 2′-dimethylaminoethoxyethoxy (also known in the art as 2′-O-dimethylaminoethoxyethyl or 2′-DMAEOE), i.e., 2′-O—CH₂—O—CH₂—N(CH₂)₂, also described in examples herein below.

Other modifications can include 2′-methoxy (2′-OCH₃), 2′-aminopropoxy (2′-OCH₂CH₂CH₂NH₂) and 2′-fluoro (2′-F). Similar modifications may also be made at other positions on the siRNA, particularly the 3′ position of the sugar on the 3′ terminal nucleotide or in 2′-5′ linked siRNAs and the 5′ position of 5′ terminal nucleotide. siRNAs may also have sugar mimetics such as cyclobutyl moieties in place of the pentofuranosyl sugar. Representative U.S. patents that teach the preparation of such modified sugar structures include, but are not limited to, U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; and 5,700,920, each of which is incorporated by reference in its entirety for all purposes.

siRNAs may also include nucleobase (often referred to in the art simply as “base”) modifications or substitutions. As used herein, “unmodified” or “natural” nucleobases include the purine bases adenine (A) and guanine (G), and the pyrimidine bases thymine (T), cytosine (C) and uracil (U). Modified nucleobases include other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl anal other 8-substituted adenines and guanines, 5-halo, particularly 5-bromo, 5-trifluoromethyl and other 5-substituted uracils and cytosines, 7-methylguanine and 7-methyladenine, 8-azaguanine and 8-azaadenine, 7-deazaguanine and 7-daazaadenine and 3-deazaguanine and 3-deazaadenine. Further nucleobases include those disclosed in U.S. Pat. No. 3,687,808, those disclosed in The Concise Encyclopedia Of Polymer Science And Engineering, pages 858-859, Kroschwitz, J. L, ed. John Wiley & Sons, 1990, these disclosed by Englisch et al., Angewandte Chemie, International Edition, 1991, 30, 613, and those disclosed by Sanghvi, Y S., Chapter 15, DsRNA Research and Applications, pages 289-302, Crooke, S. T. and Lebleu, B., Ed., CRC Press, 1993, each of which is incorporated by reference in its entirety for all purposes. Certain of these nucleobases are particularly useful for increasing the binding affinity of the oligomeric compounds featured in the invention. These include 5-substituted pyrimidines, 6-azapyrimidines and N-2, N-6 and 0-6 substituted purines, including 2-aminopropyladenine, 5-propynyluracil and 5-propynylcytosine. 5-methylcytosine substitutions have been shown to increase nucleic acid duplex stability by 0.6-1.2° C. (Sanghvi, Y. S., Crooke, S. T. and Lebleu, B., Eds., DsRNA Research and Applications, CRC Press, Boca Raton, 1993, pp. 276-278, which is incorporated by reference in its entirety for all purposes) and are exemplary base substitutions, even more particularly when combined with 2′-O-methoxyethyl sugar modifications.

Representative U.S. patents that teach the preparation of certain of the above noted modified nucleobases as well as other modified nucleobases include, but are not limited to, the above noted U.S. Pat. No. 3,687,808, as well as U.S. Pat. Nos. 4,845,205; 5,130,30; 5,134,066; 5,175,273; 5,367,066; 5,432,272; 5,457,187; 5,459,255; 5,484,908; 5,502,177; 5,525,711; 5,552,540; 5,587,469; 5,594,121, 5,596,091; 5,614,617; 5,681,941, and U.S. Pat. No. 5,750,692, each of which is incorporated by reference in its entirety for all purposes.

Another modification of the siRNAs can involve chemically linking to the siRNA one or more moieties or conjugates which enhance the activity, cellular distribution or cellular uptake of the siRNA. Such moieties include but are not limited to lipid moieties such as a cholesterol moiety (Letsinger et al., Proc. Natl. Acid. Sci. USA, 1989, 86:6553-6556), cholic acid (Manoharan et al., Biorg. Med. Chem. Let., 1994, 4:1053-1060), a thioether, e.g., beryl-S-tritylthiol (Manoharan et al., Ann. N.Y. Acad. Sci., 1992, 660:306-309; Manoharan et al., Biorg. Med. Chem. Let., 1993, 3:2765-2770), a thiocholesterol (Oberhauser et al., Nucl. Acids Res., 1992, 20:533-538), an aliphatic chain, e.g., dodecandiol or undecyl residues (Saison-Behmoaras et al., EMBO J, 1991, 10:1111-1118; Kabanov et al., FEBS Lett., 1990, 259:327-330; Svinarchuk et al., Biochimie, 1993, 75:49-54), a phospholipid, e.g., di-hexadecyl-rac-glycerol or triethyl-ammonium 1,2-di-O-hexadecyl-rac-glycero-3-Hphosphonate (Manoharan et al., Tetrahedron Lett., 1995, 36:3651-3654; Shea et al., Nucl. Acids Res., 1990, 18:3777-3783), a polyamine or a polyethylene glycol chain (Manoharan et al., Nucleosides & Nucleotides, 1995, 14:969-973), or adamantane acetic acid (Manoharan et al., Tetrahedron Lett., 1995, 36:3651-3654), a palmityl moiety (Mishra et al., Biochim. Biophys. Acta, 1995, 1264:229-237), or an octadecylamine or hexylamino-carbonyloxycholesterol moiety (Crooke et al., J. Pharmacol. Exp. Ther., 1996, 277:923-937), each of the foregoing references are incorporated by reference in its entirety for all purposes.

Representative U.S. patents that teach the preparation of such siRNA conjugates include, but are not limited to, U.S. Pat. Nos. 4,828,979; 4,948,882; 5,218,105; 5,525,465; 5,541,313; 5,545,730; 5,552,538; 5,578,717, 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,214,136; 5,245,022; 5,254,469; 5,258,506; 5,262,536; 5,272,250; 5,292,873; 5,317,098; 5,371,241, 5,391,723; 5,416,203, 5,451,463; 5,510,475; 5,512,667; 5,514,785; 5,565,552; 5,567,810; 5,574,142; 5,585,481; 5,587,371; 5,595,726; 5,597,696; 5,599,923; 5,599,928 and 5,688,941, each of which is herein incorporated by reference in its entirety for all purposes.

It is not necessary for all positions in a given compound to be uniformly modified, and in fact more than one of the aforementioned modifications may be incorporated in a single compound or even at a single nucleoside within an siRNA. The present invention also includes dsRNA compounds which are chimeric compounds. “Chimeric” siRNA compounds or “chimeras,” in the context of this invention, are siRNA compounds, particularly siRNAs, which contain two or more chemically distinct regions, each made up of at least one monomer unit, i.e., a nucleotide in the case of an siRNA compound. These siRNAs typically contain at least one region wherein the siRNA is modified so as to confer upon the siRNA increased resistance to nuclease degradation, increased cellular uptake, and/or increased binding affinity for the target nucleic acid. An additional region of the siRNA may serve as a substrate for enzymes capable of cleaving RNA:DNA or RNA:RNA hybrids. By way of example, RNase His a cellular endonuclease which cleaves the RNA strand of an RNA:DNA duplex. Activation of RNase H, therefore, results in cleavage of the RNA target, thereby greatly enhancing the efficiency of siRNA inhibition of gene expression. Consequently, comparable results can often be obtained with shorter siRNAs when chimeric siRNAs are used, compared to phosphorothioate deoxysiRNAs hybridizing to the same target region.

In certain instances, the siRNA may be modified by a non-ligand group. A number of non-ligand molecules have been conjugated to siRNAs in order to enhance the activity, cellular distribution or cellular uptake of the siRNA, and procedures for performing such conjugations are available in the scientific literature. Such non-ligand moieties have included lipid moieties, such as cholesterol (Letsinger et al., Proc. Natl. Acad. Sci. USA, 1989, 86:6553), cholic acid (Manoharan et al., Bioorg. Med. Chem. Lett., 1994, 4:1053), a thioether, e.g., hexyl-S-tritylthiol (Manoharan et al., Ann. N.Y. Acad. Sci., 1992, 660:306; Manoharan et al., Bioorg. Med. Chem. Let., 1993, 3:2765), a thiocholesterol (Oberhauser et al., Nucl. Acids Res., 1992, 20:533), an aliphatic chain, e.g., dodecandiol or undecyl residues (Saison-Behmoaras et al., EMBO J., 1991, 10:111; Kabanov et al., FEBS Lett., 1990, 259:327; Svinarchuk et al., Biochimie, 1993, 75:49), a phospholipid, e.g., di-hexadecyl-rac-glycerol or triethylammonium 1,2-di-O-hexadecyl-rac-glycero-3-H-phosphonate (Manoharan et al., Tetrahedron Lett., 1995, 36:3651; Shea et al., Nucl. Acids Res., 1990, 18:3777), a polyamine or a polyethylene glycol chain (Manoharan et al., Nucleosides & Nucleotides, 1995, 14:969), or adamantane acetic acid (Manoharan et al., Tetrahedron Lett., 1995, 36:3651), a palmityl moiety (Mishra et al., Biochim. Biophys. Acta, 1995, 1264:229), or an octadecylamine or hexylamino-carbonyl-oxycholesterol moiety (Crooke et al., J. Pharmacol. Exp. Ther., 1996, 277:923), each of the foregoing references is incorporated by reference in its entirety for all purposes. Representative United States patents that teach the preparation of such siRNA conjugates have been listed above. Typical conjugation protocols involve the synthesis of siRNAs bearing an aminolinker at one or more positions of the sequence. The amino group is then reacted with the molecule being conjugated using appropriate coupling or activating reagents. The conjugation reaction may be performed either with the siRNA still bound to the solid support or following cleavage of the siRNA in solution phase. Purification of the siRNA conjugate by HPLC typically affords the pure conjugate.

In some embodiments, the disclosed siRNA molecules are used for reducing EPO and/or EpoR activity to reduce tumor mass and increase survival in a subject with cancer or suspected of. having cancer.

In certain embodiments the disclosed siRNA is a composition that comprises RNA interference (RNAi) molecules. In some embodiments, said RNAi binds to an RNA molecule that is selected from the group consisting of an mRNA molecule that encodes an erythropoietin (EPO) protein, an mRNA molecule that encodes an EPO receptor subunit, an mRNA molecule that encodes a CD131 subunit, and any combination thereof. In some embodiments, such composition is administered to a subject to treat cancer. In some embodiments, upon administering the subject with said RNAi, tumor mass is reduced. In some embodiments, upon administering the subject with said RNAi, the immune response is increased. In some embodiments, the immune response is increased through the production of effector T (Teff) cells.

In some embodiments the RNAi is a composition administered to a subject having cancer, wherein said RNAi binds to an RNA molecule that is selected from a group consisting of an mRNA molecule that encodes a erythropoietin (EPO) protein, an mRNA molecule that encodes a EPO receptor subunit, an mRNA molecule that encodes a CD131 subunit, and any combination thereof; wherein upon administering said RNAi to said subject, the subject's tumor mass is reduced. In some embodiments, the tumor mass is reduced by at least 10%. In some embodiments, the tumor mass is reduced by at least 20%. In some embodiments, the tumor mass is reduced by at least 30%. In some embodiments, the tumor mass is reduced by at least 40%. In some embodiments, the tumor mass is reduced by at least 50%. In some embodiments, the tumor mass is reduced by at least 60%. In some embodiments, the tumor mass is reduced by at least 70%. In some embodiments, the tumor mass is reduced by at least 80%.

In some embodiments, the tumor mass is reduced to less than 0.8 cm³. In some embodiments, the tumor mass is reduced to less than 0.7 cm³. In some embodiments, the tumor mass is reduced to less than 0.6 cm³. In some embodiments, the tumor mass is reduced to less than 0.5 cm³. In some embodiments, the tumor mass is reduced to less than 0.4 cm³. In some embodiments, the tumor mass is reduced to less than 0.3 cm³. In some embodiments, the tumor mass is reduced to less than 0.2 cm³.

In some embodiments, the tumor mass is reduced to about 0.8 cm³. In some embodiments, the tumor mass is reduced to about 0.7 cm³. In some embodiments, the tumor mass is reduced to about 0.6 cm³. In some embodiments, the tumor mass is reduced to less than 0.5 cm³. In some embodiments, the tumor mass is reduced to less than 0.4 cm³. In some embodiments, the tumor mass is reduced to less than 0.3 cm³. In some embodiments, the tumor mass is reduced to less than 0.2 cm³.

In some embodiments the RNAi is a composition administered to a subject having cancer, wherein said RNAi binds to an RNA molecule that is selected from a group consisting of an mRNA molecule that encodes an erythropoietin (EPO) protein, an mRNA molecule that encodes an EPO receptor subunit, an mRNA molecule that encodes a CD131 subunit, and any combination thereof; wherein upon administering said RNAi to said subject, the subject's immune response is increased through the production of more effector T (Teff) cells.

In some embodiments, the targeted cancer is selected from hepatocarcinoma, colon cancer, breast cancer, lung cancer, brain cancer, or melanoma.

In some embodiments, the RNAi molecules reduce EPO half-life in a subject. In some embodiments, the RNAi molecules reduce EPO levels in a subject. In some embodiments, reduced EPO levels increases survival. In some embodiments, the survival rate is increased two-fold. In some embodiments, the survival rate is increased three-fold. In some embodiments, the survival rate is increased five-fold. In some embodiments, the survival rate is increased by about half a year to about 5 years. In some embodiments, the survival rate is increased by about half a year to about 3 years. In some embodiments, the survival rate is increased by about half a year to about a year.

In some embodiments, the RNAi is in a nanoparticle. Any suitable nanoparticle described herein will be a useful nanoparticle carrier. In some embodiments, the nanoparticle is a lipid nanoparticle. In some embodiments, the lipid nanoparticle comprises about 20-70% cationic lipid:about 5-45% neutral lipid:about 20-55% cholesterol; and/or about 0.5-15% PEG-modified lipid. In some embodiments, the lipid nanoparticle comprises about 20-60% cationic lipid:about 5-25% neutral lipid:about 25-55% cholesterol; and/or about 0.5-15% PEG-modified lipid. In some embodiments, the lipid nanoparticle comprises about 35 to 45% cationic lipid, about 40% to 50% cationic lipid, about 50% to 60% cationic lipid, and/or about 55% to 65% cationic lipid.

In some embodiments, the ratio of the RNAi to lipid nanoparticles is about 5:1 to about 20:1. In some embodiments, the ratio of the RNAi to lipid nanoparticles is about 10:1 to about 25:1. In some embodiments, the ratio of the RNAi to lipid nanoparticles is about 15:1 to about 30:1. In some embodiments, the ratio of the RNAi to lipid nanoparticles is at least about 30:1.

In some embodiments, the RNAi is an siRNA, or an miRNA, or an antisense RNA, or a lncRNA. In some embodiments, the RNAi is an siRNA. In some embodiments the RNAi is an miRNA. In some embodiments, the RNAi is an antisense RNA. In some embodiments, the RNAi is a lncRNA.

In some embodiments, an siRNA has a sequence length of about 3 to about 90 nucleotides. In some embodiments, an siRNA has a sequence length of about 3 to about 60 nucleotides. In some embodiments, an siRNA has a sequence length of about 3 to about 45 nucleotides. In some embodiments, an siRNA has a sequence length of about 9 to about 42 nucleotides. In some embodiments, an siRNA has a sequence length of about 15 to about 30 nucleotides. In some embodiments, an siRNA has a sequence length of about 21 to about 30 nucleotides.

In some embodiments, an siRNA molecule comprises a nucleic acid that is at least 70%, 75%, 80%, 85%, 90%, 95%, 97%, 99%, 100% identical to any of the following sequences: SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 21, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 42, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, or SEQ ID NO: 64.

In some embodiments, the siRNA targets the following sequences: SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, or SEQ ID NO: 21.

In some embodiments, the siRNA comprises a nucleic acid that is at least 70%, 75%, 80%, 85%, 90%, 95%, 97%, 99%, 100% identical to any the following sequences: SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, or SEQ ID NO: 42.

In some embodiments, the siRNA comprises a nucleic acid that is at least 70%, 75%, 80%, 85%, 90%, 95%, 97%, 99%, 100% identical to any of the following sequences: SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, or SEQ ID NO: 64.

Small molecules can also be used to upregulate or downregulate EPO and/or EPOR, so as to induce immunotolerance or immunosuppression (collectively negative immune modulation), or increase immune activity. For example, inhibitors of hypoxia-inducible factor (HIF) can reduce EPO/EPOR mediated immunosuppression, and inhibitors of HIF-prolyl hydroxylase (PHD) can stimulate immune tolerance or immunosuppression.

Hypoxia-inducible factor (HIF) is a helix-loop-helix transcription factor that acts as a master regulator of hypoxia activated gene expression, allowing adaptation to hypoxia. HIF is a heterodimer complex composed by two subunits, α-subunit (oxygen sensitive) and a β-subunit (constitutively expressed, and also called aryl hydrocarbon receptor nuclear translocator (ARNT)). HIF-prolyl hydroxylase (PHD) leads to degradation of HIF and is a O2-sensitive negative regulator of HIF. Hence PHD inhibitors lead to activation of HIF signaling. PHD inhibitors can increase HIF activity and this can stimulate erythropoiesis.

The HIF pathway along with the HIF-prolyl hydroxylase domain (PHD) are transcription factors that are important oxygen-sensing pathways for mediating tissue adaptation to low oxygen environments primarily by the transcription regulation of gene expression. Inhibitors of HIF can reduce EPO/EPOR mediated immunosuppression, and inhibitors of PHD can stimulate the immune tolerance or immunosuppression. EPO is typically present in low amounts in circulation under homeostatic conditions. When erythropoietic stress occurs through hypoxia or anemia, it can result in a dramatic increase in EPO production. Since hypoxia is a significant feature in many cancers and some chronic conditions, inhibition of the HIF transcription factor can promote reduction of tumor growth or alleviating and or treating chronic conditions.

The HIF transcription factor has an oxygen-sensitive α-subunit and a constantly expressed β-subunit. Three HIF-α subunits are currently known: HIF-1α, HIF-2α, and HIF-3α. Under hypoxic conditions, the α-subunit no longer degrades, and will form a heterodimer with the β-subunit, which activates gene transcription. By inhibiting the heterodimer formation, gene transcription is not activated, which can result in inhibiting the cellular response to hypoxia including inhibition of EPO production.

The inhibitor of HIF inhibits HIF activity by inhibiting the HIF pathway activity indirectly by a variety of mechanisms. The inhibitors of HIF can inhibit HIF-1α protein synthesis, HIF-1α protein stabilization, HIF-1α-HIF-1β dimerization, and HIF-1 dimer DNA binding and interactions with other proteins. In some embodiments, the inhibitor of HIF is a HIF-1 inhibitor.

The HIF and PHD pathways coordinate the hypoxia responses for cells and tissues. PHD is a 2-oxoglutarate (2OG)-dependent oxygenase which utilizes molecular oxygen for various cellular processes including HIF regulation and hypoxia response.

Inhibitors of PHD are useful for activating the HIF pathway by impairing HIF-α degradation, which leads to HIF signaling. The HIF signaling can stimulate the erythropoiesis protein (EPO), which can enhance apoptotic cell clearance and immune tolerance.

Other pathways that can regulate EPO include interleukin pathways (such as IL-1α, IL-1β, and IL-6), tumor necrosis factor (TNF-α), estrogen receptors, Phospholipase C, gamma 1 (phospholipase C-γ1), and Cb1/p85/Episin-1 pathway.

Interleukins are cytokines expressed and secreted by white blood cells that regulate immune responses, inflammatory responses, and hematopoiesis. The inhibition of certain interleukins can suppress the actions of interleukins for the immune system, which results in antagonistic effects of EPO production, and inhibiting hetero-EPOR activity.

TNF-α is an adipokine and cytokine which regulates immune cells. Inhibitors of TNF-α can reduce the levels of EPO induced cell proliferation and inhibit hetero EPOR activity.

Estrogen receptors are proteins found inside cells and activated by the hormone estrogen. After estrogen activation, the estrogen receptors can translocate to the nucleus and bind DNA to regulate the activity of different genes. Activation of estrogen receptors has also been found to promote cell proliferation, and in breast cancer cells with estrogen receptors, there have been found functional EPO receptors as well. Inhibitors and antagonists of estrogen receptors can inhibit cellular proliferation and inhibit EPOR promotion of cell growth.

Phospholipase C-γ1 is a cell growth factor protein that is involved in cell growth, migration, proliferation, and apoptosis. Mutations of this cell growth factor can lead to tumor growth via cancer cell proliferation. Additionally, EPO can induce activation of Phospholipase C-γ1. Inhibitors of phospholipase C-γ1 can inhibit the activation of phospholipase C-γ1, thereby inhibiting hetero-EPOR activity to reduce tumor growth.

The Cb1/p85/Episin-1 pathway can mediate EPO-induced EPOR internalization, and thereby reduce EPO signaling. EPO can induce Cb1-dependent ubiquination of the p85 regulatory subunit, which results in binding of phosphotyrosinases on EPOR. This results in endocytosis of EPOR. Cb1 is an E3 ligase which plays a role in endocytic downregulation of receptor tyrosine kinases. Promotion of Cb1/p85 activation can result in endocytosis of EPOR, which reduces EPOR activity.

In some embodiments, small molecules are used to downregulate EPO, so as to induce immunosuppression (collectively negative immune modulation) or increase immune activity.

In some embodiments a composition is administered to a subject having cancer or chronic diseases, comprising a compound, a pharmaceutically acceptable salt, solvate, or steroisomer thereof, wherein said compound inhibits a hetero-erythropoietin (EPO) receptor activity in a myeloid cell in said subject.

In some embodiments a composition is administered to a subject having cancer or chronic infection condition, comprising a compound, a pharmaceutically acceptable salt, solvate, or stereoisomer thereof, wherein said compound inhibits a hetero-erythropoietin (EPO) receptor activity in a myeloid cell in said subject.

Chronic diseases are diseases which persist with long-lasting effects on a subject. Chronic diseases may have remission periods, wherein the disease temporarily goes away, or reappears. Chronic diseases can be alleviated by altering dietary, lifestyle and metabolic risk factors of a subject. These are behavioral changes which can be performed by the subject. Chronic diseases can also be treated using the compounds described herein. Chronic diseases can be broadly categorized into two categories, chronic infectious diseases or conditions and chronic-non-communicable diseases.

Chronic infectious diseases are chronic conditions which are caused by transmissible infections. Examples of chronic infection diseases include, but is not limited to human immunodeficiency virus infection and acquired immunodeficiency syndrome (HIV/AIDS), tuberculosis (TB), Lyme diseases, and graft-versus-host disease.

Chronic non-communicable diseases include, but is not limited to cancers, cardiovascular diseases, chronic respiratory diseases, and diabetes mellitus. Other diseases include but are not limited to Alzheimer's disease, Huntington's disease, Parkinson's disease, autoimmune diseases, chronic hepatitis, and chronic kidney diseases.

In some embodiments a composition is administered to a subject having cancer or chronic infection condition, comprising a compound, a pharmaceutically acceptable salt, solvate, or stereoisomer thereof, wherein said compound inhibits a hetero-erythropoietin (EPO) receptor activity so that resistance to immune-checkpoint blockade is reversed in said subject.

In some embodiments, the hetero-EPO receptor comprises an EPO subunit and a CD131 subunit. In some embodiments, the hetero-EPO receptor is on a macrophage, monocyte, dendritic cell, basophil, neutrophil, or eosinophil.

In some embodiments, a composition comprised of a compound, or pharmaceutically acceptable salt, solvate, or stereoisomer thereof, inhibits hetero-erythropoietin (EPO) receptor's activity. In some embodiments, such composition can treat cancer or chronic infection condition in a subject. In some embodiments, the cancer is selected from hepatocarcinoma, colon cancer, breast cancer, lung cancer, brain cancer, or melanoma. In another embodiment, the chronic infectious condition develops in patients with an organ transplant or skin grafting.

In some embodiments, the inhibitory activity occurs in a myeloid cell. In another embodiment, the inhibitory activity results in reversal of resistance to immune-checkpoint blockade. In some embodiments, an inhibitory activity leads to a decrease of a cancer cell population.

In some embodiments, the immune-checkpoint blockade is an inhibitor of CTLA-4, PD-1, or PD-L1. In some embodiments, the immune-checkpoint blockade is an inhibitor of PD-1 or PD-L1. In some embodiments, the immune-checkpoint blockade is an inhibitor of CTLA-4. In some embodiments, the inhibitor of CTLA-4, PD-1, or PD-L1 is Nivolumab, Pembrolizumab, Cemiplimab, Atezolizumab, Avelumab, Durvalumab, Ipilimumab, Lirilumab, and BMS-986016.

In some embodiments, the compound is an inhibitor of hypoxia-inducible factor (HIF), IL-1α, IL-1β, TNF-α, IL-6, estrogen receptors, phospholipase C-γ1, or Cb1/p85/Episin-1 pathway. In some embodiments, the compound is an inhibitor of hypoxia-inducible factor (HIF), IL-1α, IL-1β, TNF-α, IL-6, or estrogen receptors. In some embodiments, the compound is an inhibitor of hypoxia-inducible factor (HIF).

In some embodiments, the compound is selected from CAY10585 (LW6), Chetomin, Chrysin, Dimethyl-bisphenol A, Echinomycin, 2-Methoxyestradiol (2ME2), SYP-5, PX-478 2HCl, KC7F2, GN44028, Verucopeptin, FM19G11, PT2399, PT2385, Belzutifan, HIF-2a-IN-1, HIF-2a-IN-2, HIF-2a-IN-3, HIF-2a-IN-4, TC-S 700, IDF-11774, Paeoniflorin, Emetine hydrochloride, Glucosamine, PX12, Vitexin, BAY 87-2243, Lificiguat (YC-1), Vorinostat, Tanespimycin, Silibinin, diallyl trisulfide (DATS), Herboxidiene (GEX1A), Celastrol, Phenethyl isothiocyanate (PE1TC), Gliotoxin, Sulforaphane, Acriflavin, Emodin, Cardenolide, 3,3′-Diindolylmethane (DIM), Pseudolaric acid-B (PAB), Bavachinin, Andrographolide, Isoliquiritigenin, Wondonin, Thymoquinone, or Curcumin.

In some embodiments, the compound is CAY10585 (LW6), Chetomin, Chrysin, Dimethyl-bisphenol A, Echinomycin, 2-Methoxyestradiol (2ME2), SYP-5, PX-478 2HCl, KC7F2, GN44028, Verucopeptin, FM19G11, PT2399, PT2385, Belzutifan, HIF-2a-IN-1, HIF-2a-IN-2, HIF-2a-IN-3, HIF-2a-IN-4, TC-S 700, IDF-11774, Paconiflorin, Emetine hydrochloride, Glucosamine, PX12, Vitexin, BAY 87-2243, Lificiguat (YC-1), Vorinostat, or Tanespimycin.

In certain embodiments, the compound is Chetomin, Echinomycin, PT2399, Belzutifan, Vorinostat, or Tanespimycin.

In some embodiments, said compound is selected from Silibinin, diallyl trisulfide (DATS), Herboxidiene (GEX1A), Celastrol, Phenethyl isothiocyanate (PEITC), Gliotoxin, Sulforaphane, Acriflavin, Emodin, Cardenolide, 3,3′-Diindolylmethane (DIM), Pseudolaric acid-B (PAB), Bavachinin, Andrographolide, Isoliquiritigenin, Wondonin, Thymoquinone, or Curcumin.

In some embodiments, small molecules can also be used to upregulate EPOR, so as to induce immunotolerance.

In some embodiments, a composition comprising of a compound, or pharmaceutically acceptable salt, solvate, or stereoisomer thereof, promotes hetero-erythropoietin (EPO) receptor's activity. In some embodiments, immune tolerance to an antigen is increased in a subject exposed to such a composition. In certain embodiments, a compound has no substantial effect on EPO receptor activity. In some embodiments, the EPO receptor comprises at least two EPO receptor subunits.

In some embodiments is a composition for administering to a subject, comprising a compound, a pharmaceutically acceptable salt, solvate, or stereoisomer thereof, wherein said compound promotes a hetero-erythropoietin (EPO) receptor activity, wherein said hetero-EPO receptor comprises a EpoR subunit and CD131 subunit, so that immune tolerance to an antigen is increased in said subject; and wherein said compound has no substantial effect on a homo-EPO receptor activity, wherein said homo-EPO receptor comprises at least two EPO receptor subunits.

In some embodiments, the compound is an inhibitor of HIF-Prolyl Hydroxylase (PHD), NHF-4, GATA factor, IL-17, AKT/NFkB/HIF1 pathway, estrogen receptor, Angiotensin II receptor, Topoisomerase II, or Epithelial membrane protein 1 (EMP-1).

In some embodiments, the compound is an inhibitor of HIF-Prolyl Hydroxylase (PHD), NHF-4, GATA factor, Angiotensin II receptor, Topoisomerase II, or IL-17.

In some embodiments, the compound is an inhibitor of HIF-Prolyl Hydroxylase (PHD).

In some embodiments, the compound is selected from Roxadustat, Vadadustat, Enarodustat, Desidustat, Molidustat, Dimethyloxaloylglycine, Daprodustat, Prolyl Hydroxylase inhibitor 1, TM6089, TRC160334, PHD-1-IN-1, MK-8617, JNJ-42041935, TP0463518, IOX (JICL38), IOX4, IOX3 (FG-2216), Dencichin, HIF-PHD-IN-1, AKB-6899, VH298, M1001, ML228, Dimethyloxalylglycine (DMOG), Mitoxantrone, Angiotensin II (Ang II), or 17β-estradiol.

In another embodiments, the compound is selected from Roxadustat, Vadadustat, Enarodustat, Desidustat, Molidustat, Dimethyloxaloylglycine, Daprodustat, Prolyl Hydroxylase inhibitor 1, TM6089, TRC160334, PHD-1-IN-1, MK-8617, JNJ-42041935, TP0463518, IOX (JICL38), IOX4, IOX3 (FG-2216), Dencichin, HIF-PHD-IN-1, AKB-6899, VH298, M1001, ML228, Dimethyloxalylglycine (DMOG).

In some embodiments, the compound is Mitoxantrone, Angiotensin II (Ang II), or 17β-estradiol.

### Pharmaceutical Compositions

Additional embodiments of the disclosure relate to pharmaceutical compositions comprising an anti-EPOR antibody and/or anti-CD131 antibody and/or anti-EPO antibody and/or EPO analog, or a pharmaceutically acceptable salt, solvate or hydrate thereof, and one or more pharmaceutically acceptable excipients or carriers. The compositions can optionally contain an additional therapeutic agent. In general, a pharmaceutical composition comprises a therapeutically effective amount of an anti-EPOR antibody and/or an anti-CD131 antibody and/or an anti-EPO antibody and/or an EPO analog, one or more pharmaceutically acceptable excipients or carriers and optionally a therapeutically effective amount of an additional therapeutic agent, and is formulated for administration to a subject for therapeutic use.

Pharmaceutical compositions generally can be prepared according to current good manufacturing practice (GMP), as recommended or required by, e.g., the Federal Food, Drug, and Cosmetic Act § 501 (a)(2)(B) and the International Conference on Harmonisation Q7 Guideline.

Pharmaceutical compositions/formulations can be prepared in sterile forms. For example, pharmaceutical compositions/formulations for parenteral administration by injection or infusion generally are sterile. Sterile pharmaceutical compositions/formulations can be compounded or manufactured according to pharmaceutical-grade sterilization standards known to those of skill in the art, such as those disclosed in or required by the United States Pharmacopeia Chapters 797, 1072 and 1211, and 21 Code of Federal Regulations 211.

Pharmaceutically acceptable excipients and carriers can include pharmaceutically acceptable substances, materials and/or vehicles. Non-limiting examples of types of excipients can include liquid and solid fillers, diluents, binders, lubricants, glidants, surfactants, dispersing agents, disintegration agents, emulsifying agents, wetting agents, suspending agents, thickeners, solvents, isotonic agents, buffers, pH adjusters, absorption-delaying agents, stabilizers, antioxidants, preservatives, antimicrobial agents, antibacterial agents, antifungal agents, chelating agents, adjuvants, sweetening agents, flavoring agents, coloring agents, encapsulating materials, and coating materials. The use of such excipients in pharmaceutical formulations is known in the art. For example, conventional vehicles and carriers can include, but are not limited to, oils (e.g., vegetable oils such as olive oil and sesame oil), aqueous solvents {e.g., saline, buffered saline (e.g., phosphate-buffered saline [PBS]) and isotonic solutions (e.g., Ringer's solution)}, and organic solvents (e.g., dimethyl sulfoxide [DMSO] and alcohols [e.g., ethanol, glycerol and propylene glycol]). Except insofar as any conventional excipient or carrier is incompatible with an anti-EPOR antibody, an anti-CD131 antibody, an anti-EPO antibody, an EPO analog, or a fragment thereof, the disclosure encompasses the use of conventional excipients and carriers in formulations containing an anti-EPOR antibody, an anti-CD131 antibody, an anti-EPO antibody, an EPO analog, or a fragment thereof. See, e.g., Remington: The Science and Practice of Pharmacy, 21st Ed., Lippincott Williams & Wilkins (Philadelphia, Pennsylvania) (2005); Handbook of Pharmaceutical Excipients, 5th Ed., Rowe et al., Eds., The Pharmaceutical Press and the American Pharmaceutical Association (2005); Handbook of Pharmaceutical Additives, 3rd Ed., Ash and Ash, Eds., Gower Publishing Co. (2007); and Pharmaceutical Pre-formulation and Formulation, Gibson, Ed., CRC Press (Boca Raton, Florida) (2004).

Appropriate formulation can depend on various factors, such as the route of administration chosen. Potential routes of administration of a pharmaceutical composition comprising an anti-EPOR antibody and/or an anti-CD131 antibody and/or an anti-EPO antibody and/or an EPO analog can include, but are not limited to, oral, parenteral (including intradermal, subcutaneous, intramuscular, intravascular, intravenous, intraarterial, intraperitoneal, intramedullary, intrathecal and topical), intracavitary, and topical (including dermal/epicutaneous, transdermal, mucosal, transmucosal, intranasal [e.g., by nasal spray or drop], intraocular [e.g., by eye drop], pulmonary [e.g., by oral or nasal inhalation], buccal, sublingual, rectal [e.g., by suppository], and vaginal [e.g., by suppository]). Topical formulations can be designed to produce a local or systemic therapeutic effect. In certain embodiments, an anti-EPOR antibody and/or an anti-CD131 antibody and/or an anti-EPO antibody and/or an EPO analog, or a fragment thereof can be administered parenterally (e.g., intravenously, subcutaneously, intramuscularly or intraperitoneally) by injection (e.g., as a bolus) or by infusion over a period of time.

Excipients and carriers that can be used to prepare parenteral formulations can include, but are not limited to, solvents (e.g., aqueous solvents such as water, saline, physiological saline, buffered saline [e.g., phosphate-buffered saline], balanced salt solutions [e.g., Ringer's BSS] and aqueous dextrose solutions), isotonic/iso-osmotic agents (e.g., salts [e.g., NaCl, KCl and CaCl₂)] and sugars [e.g., sucrose]), buffering agents and pH adjusters (e.g., sodium dihydrogen phosphate [monobasic sodium phosphate]/disodium hydrogen phosphate [dibasic sodium phosphate], citric acid/sodium citrate and L-histidine/L-histidine HCl), and emulsifiers (e.g., non-ionic surfactants such as polysorbates [e.g., polysorbate 20 and 80] and poloxamers [e.g., poloxamer 188]). Protein formulations and delivery systems are discussed in, e.g., A. J. Banga, Therapeutic Peptides and Proteins: Formulation, Processing, and Delivery Systems, 3rd Ed., CRC Press (Boca Raton, Florida) (2015).

The excipients can optionally include one or more substances that increase protein stability, increase protein solubility, inhibit protein aggregation, or reduce solution viscosity, or any combination or all thereof. Examples of such substances can include, but are not limited to, hydrophilic amino acids (e.g., arginine and histidine), polyols (e.g., myo-inositol, mannitol and sorbitol), saccharides {e.g., glucose (including D-glucose [dextrose]), lactose, sucrose and trehalose}, osmolytes (e.g., trehalose, taurine, amino acids [e.g., glycine, sarcosine, alanine, proline, serine, β-alanine and γ-aminobutyric acid], and betaines [e.g., trimethylglycine and trimethylamine N-oxide]), and non-ionic surfactants {e.g., alkyl polyglycosides, ProTek® alkylsaccarides (e.g., a monosaccharide [e.g., glucose] or a disaccharide [e.g., maltose or sucrose] coupled to a long-chain fatty acid or a corresponding long-chain alcohol), and polypropylene glycol/polyethylene glycol block co-polymers (e.g., poloxamers [e.g., Pluronic™ F-68], and Genapol® PF-10 and variants thereof)}. Because such substances can increase protein solubility, these substances can be used to increase protein concentration in a formulation. Higher protein concentration in a formulation can be advantageous for subcutaneous administration, which has a limited volume of bolus administration (e.g., ≤about 1.5 mL). In addition, such substances can be used to stabilize proteins during the preparation, storage and reconstitution of lyophilized proteins.

For parenteral (e.g., intravenous, subcutaneous or intramuscular) administration, a sterile solution or suspension of an anti-EPOR antibody and/or an anti-CD131 antibody and/or an anti-EPO antibody and/or an EPO analog in an aqueous solvent containing one or more excipients can be prepared beforehand and can be provided in, e.g., a pre-filled syringe. Alternatively, an anti-EPOR antibody and/or an anti-CD131 antibody and/or an anti-EPO antibody and/or an EPO analog can be dissolved or suspended in an aqueous solvent that can optionally comprise one or more excipients prior to lyophilization (freeze-drying). Shortly prior to parenteral administration, the lyophilized anti-EPOR antibody and/or anti-CD131 antibody and/or anti-EPO antibody and/or EPO analog stored in a suitable container (e.g., a vial) can be reconstituted with, e.g., sterile water that can optionally comprise one or more excipients. If the anti-EPOR antibody and/or anti-CD131 antibody and/or anti-EPO antibody and/or EPO analog is to be administered by infusion (e.g., intravenously), the solution or suspension of the reconstituted anti-EPOR antibody and/or anti-CD131 antibody and/or anti-EPO antibody and/or EPO analog can be added to and diluted in an infusion bag containing, e.g., sterile saline (e.g., about 0.9% NaCl).

Excipients that can enhance transmucosal penetration of smaller proteins include, but are not limited to, cyclodextrins, alky saccharides (e.g., alkyl glycosides and alkyl maltosides [e.g., tetradecylmaltoside]), and bile acids (e.g., cholic acid, glycocholic acid, taurocholic acid, deoxycholic acid, glycodeoxycholic acid, chenodeoxycholic acid and dehydrocholic acid).

Excipients that can enhance transepithelial or transdermal penetration of smaller proteins include, but are not limited to, chemical penetration enhancers (CPEs, including fatty acids [e.g., oleic acid]), cell-penetrating peptides {CPPs, including arginine-rich CPPs [e.g., polyarginines such as R₆-R₁₁ (e.g., R₆ and R₉) and TAT-related CPPs such as TAT (49-57)] and amphipathic CPPs [e.g., Pep-1 and penetratin]}, and skin-penetrating peptides (SPPs, such as the skin-penetrating and cell-entering [SPACE] peptide). Transdermal penetration of smaller proteins can be further enhanced by use of a physical enhancement technique, such as iontophoresis, cavitational or non-cavitational ultrasound, electroporation, thermal ablation, radio frequency, microdermabrasion, microneedles or jet injection. US 2007/0269379 provides an extensive list of CPEs. F. Milletti, Drug Discov. Today, 17:850-860 (2012) is a review of CPPs. R. Ruan et al., Ther. Deliv., 7:89-100 (2016) discuss CPPs and SPPs for transdermal delivery of macromolecules, and M. Prausnitz and R. Langer, Nat. Biotechnol., 26:1261-1268 (2008) discuss a variety of transdermal drug-delivery methods.

An anti-EPOR antibody and/or an anti-CD131 antibody and/or an anti-EPO antibody and/or an EPO analog can be delivered from a sustained-release composition. As used herein, the term “sustained-release composition” can encompass sustained-release, prolonged-release, extended-release, slow-release and controlled-release compositions, systems and devices. Protein delivery systems are discussed in, e.g., Banga (supra). A sustained-release composition can deliver a therapeutically effective amount of an anti-EPOR antibody and/or an anti-CD131 antibody and/or an anti-EPO antibody and/or an EPO analog over a prolonged time period. In some embodiments, a sustained-release composition can deliver an anti-EPOR antibody and/or an anti-CD131 antibody and/or an anti-EPO antibody and/or EPO analog over a period of at least about 3 days, 1 week, 2 weeks, 3 weeks, 1 month (4 weeks), 6 weeks, 2 months, 3 months or longer. A sustained-release composition can be administered, e.g., parenterally (e.g., intravenously, subcutaneously or intramuscularly).

A sustained-release composition of an anti-EPOR antibody and/or an anti-CD131 antibody and/or an anti-EPO antibody and/or an EPO analog can be in the form of, e.g., a particulate system, a lipid or oily composition, or an implant. Particulate systems can include, but are not limited to, nanoparticles, nanospheres, nanocapsules, microparticles, microspheres, and microcapsules. Nanoparticulate systems generally can have a diameter or an equivalent dimension smaller than about 1 μm. In certain embodiments, a nanoparticle, a nanosphere or a nanocapsule can have a diameter or an equivalent dimension of no more than about 500 nm, about 400 nm, or about 300 nm, or no more than about 200 nm, about 150 nm, or about 100 nm. In an aspect, a microparticle, a microsphere or a microcapsule can have a diameter or an equivalent dimension of about 1-200 μm, about 100-200 μm, or about 50-150 μm, or about 1-100 μm, about 1-50 μm, or about 50-100 μm. A nano- or a microcapsule can typically comprise a therapeutic agent in the central core, while the therapeutic agent typically can be dispersed throughout a nano- or a microparticle, or a sphere. In an aspect, a nanoparticulate system can be administered intravenously, while a microparticulate system can be administered subcutaneously or intramuscularly.

In an aspect, a sustained-release particulate system or implant can be made of a biodegradable polymer and/or a hydrogel. In certain embodiments, the biodegradable polymer can comprise lactic acid and/or glycolic acid [e.g., an L-lactic acid-based copolymer, such as poly(L-lactide-co-glycolide) or poly(L-lactic acid-co-D,L-2-hydroxyoctanoic acid)]. Non-limiting examples of polymers of which a hydrogel can be composed can include polyvinyl alcohol, acrylate polymers (e.g., sodium polyacrylate), and other homopolymers and copolymers having a relatively large number of hydrophilic groups (e.g., hydroxyl or/and carboxylate groups). The biodegradable polymer of the particulate system or implant can be selected so that the polymer substantially completely degrades around the time the period of treatment is expected to end, and so that the byproducts of the polymer's degradation, like the polymer, are biocompatible.

Alternatively, a sustained-release composition of a protein can be composed of a non-biodegradable polymer. Non-limiting examples of non-biodegradable polymers can include poloxamers (e.g., poloxamer 407). Sustained-release compositions of a protein can be composed of other natural or synthetic substances or materials, such as hydroxyapatite.

Sustained-release lipid or oily compositions of a protein can be in the form of, e.g., liposomes, micelles (e.g., those composed of biodegradable natural or/and synthetic polymers, such as lactosomes), or emulsions in an oil.

A sustained-release composition can be formulated or designed as a depot, which can be injected or implanted, e.g., subcutaneously or intramuscularly. A depot can be in the form of, e.g., a polymeric particulate system, a polymeric implant, or a lipid or oily composition. A depot formulation can comprise a mixture of a protein and, e.g., a biodegradable polymer [e.g., poly(lactide-co-glycolide)] or a semi-biodegradable polymer (e.g., a block copolymer of lactic acid and PEG) in a biocompatible solvent system, whether or not such a mixture forms a particulate system or implant.

A pharmaceutical composition can be presented in unit dosage form as a single dose wherein all active and inactive ingredients are combined in a suitable system, and components do not need to be mixed to form the composition to be administered. The unit dosage form can generally comprise an effective dose of the therapeutic agent. A representative example of a unit dosage form is a single-use pen comprising a pre-filled syringe, a needle and a needle cover for parenteral (e.g., intravenous, subcutaneous or intramuscular) injection of the therapeutic agent.

Alternatively, a pharmaceutical composition can be presented as a kit in which the therapeutic agent, excipients and carriers (e.g., solvents) are provided in two or more separate containers (e.g., ampules, vials, tubes, bottles or syringes) and need to be combined to form the composition to be administered. The kit can comprise instructions for storing, preparing and administering the composition (e.g., a solution to be injected intravenously or subcutaneously).

A kit can comprise all active and inactive ingredients in unit dosage form or the active ingredient and inactive ingredients in two or more separate containers, and can contain instructions for administering or using the pharmaceutical composition to treat a medical condition.

In some embodiments, a nanoparticle with a mean diameter of about 50-200 nm can be used. The nanoparticle can be a lipid nanoparticle. A lipid nanoparticle can comprise a cationic lipid, a neutral lipid, a PEG-modified lipid, a sterol, or a non-cationic lipid. In some embodiments, the lipid nanoparticle can comprise a molar ratio of about 20-60% cationic lipid, about 0.5-15% PEG-modified lipid, about 25-55% sterol, and about 25% non-cationic lipid. The cationic lipid can be an ionizable cationic lipid and the non-cationic lipid can be a neutral lipid, and/or the sterol can be a cholesterol. The cationic lipid can be selected from 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy) heptadecanedioate (L319).

A lipid nanoparticle formulation can be composed of a lipid mixture in molar ratios of about 20-70% cationic lipid:about 5-45% neutral lipid:about 20-55% cholesterol; and/or about 0.5-15% PEG-modified lipid. In some embodiments, a lipid nanoparticle formulation can be composed of a lipid mixture in a molar ratio of about 20-60% cationic lipid:about 5-25% neutral lipid:about 25-55% cholesterol; and/or about 0.5-15% PEG-modified lipid. In some embodiments, a lipid nanoparticle formulation can be composed of about 35 to 45% cationic lipid, about 40% to 50% cationic lipid, about 50% to 60% cationic lipid, and/or about 55% to 65% cationic lipid. In some embodiments, the ratio of lipid to RNA (e.g., mRNA) in lipid nanoparticles can be about 5:1 to about 20:1, about 10:1 to about 25:1, about 15:1 to about 30:1, and/or at least about 30:1.

A lipid nanoparticle formulation can include about 0.5% to about 15% on a molar basis of the neutral lipid, e.g., about 3 to 12%, about 5 to 10% or about 15%, about 10%, or about 7.5% on a molar basis. Examples of neutral lipids can include, but are not limited to, DSPC, POPC, DPPC, DOPE and SM.

The formulation can include from about 5% to about 50% on a molar basis of the sterol (e.g., about 15 to 45%, about 20 to 40%, about 40%, about 38.5%, about 35%, or about 31% on a molar basis. A non-limiting example of a sterol can include cholesterol.

A lipid nanoparticle formulation can include from about 0.5% to about 20% on a molar basis of the PEG or PEG-modified lipid (e.g., about 0.5 to 10%, about 0.5 to 5%, about 1.5%, about 0.5%, about 1.5%, about 3.5%, or about 5% on a molar basis. A PEG or PEG modified lipid comprises a PEG molecule of an average molecular weight of about 2,000 Da. A PEG or PEG modified lipid can comprise a PEG molecule of an average molecular weight of less than about 2,000 Da, for example about 1,500 Da, about 1,000 Da, or about 500 Da. Non-limiting examples of PEG-modified lipids can include PEG-distearoyl glycerol (PEG-DMG) (also referred herein as PEG-C14 or C14-PEG), and PEG-CDMA (further discussed in Reyes et al. J. Controlled Release, 107, 276-287 (2005) the contents of which are herein incorporated by reference in their entirety).

The ratio of PEG in the lipid nanoparticle formulations may be increased or decreased and/or the carbon chain length of the PEG lipid may be modified from C14 to C18 to alter the pharmacokinetics and/or biodistribution of the lipid nanoparticle formulations. As a non-limiting example, lipid nanoparticle formulations may contain from about 0.5% to about 3.0%, from about 1.0% to about 3.5%, from about 1.5% to about 4.0%, from about 2.0% to about 4.5%, from about 2.5% to about 5.0% and/or from about 3.0% to about 6.0% of the lipid molar ratio of PEG-c-DOMG (R-3-[(.omega.-methoxy-poly(ethyleneglycol)2000) carbamoyl)]-1,2-dimyristy-loxypropyl-3-amine) (also referred to herein as PEG-DOMG) as compared to the cationic lipid, DSPC and cholesterol. The PEG-c-DOMG may be replaced with a PEG lipid including, but not limited to, PEG-DSG (1,2-Distearoyl-sn-glycerol, methoxypolyethylene glycol), PEG-DMG (1,2-Dimyristoyl-sn-glycerol) and/or PEG-DPG (1,2-Dipalmitoyl-sn-glycerol, methoxypolyethylene glycol). The cationic lipid may be selected from any lipid known in the art including, but not limited to, DLin-MC3-DMA, DLin-dMA, C12-200 and DLin-KC2-DMA.

The molar lipid ratio can be 50/10/38.5/1.5 (mol % cationic lipid/neutral lipid, e.g., DSPC/Chol/PEG-modified lipid, e.g., PEG-DMG, PEG-DSG or PEG-DPG), 57.2/7.1134.3/1.4 (mol % cationic lipid/neutral lipid, e.g., DPPC/Chol/PEG-modified lipid, e.g., PEG-cDMA), 40/15/40/5 (mol % cationic lipid/neutral lipid, e.g., DSPC/Chol/PEG-modified lipid, e.g., PEG-DMG), 50/10/35/4.5/0.5 (mol % cationic lipid/neutral lipid, e.g., DSPC/Chol/PEG-modified lipid, e.g., PEG-DSG), 50/10/35/5 (cationic lipid/neutral lipid, e.g., DSPC/Chol/PEG-modified lipid, e.g., PEG-DMG), 40/10/40/10 (mol % cationic lipid/neutral lipid, e.g., DSPC/Chol/PEG-modified lipid, e.g., PEG-DMG or PEG-cDMA), 35/15/40/10 (mol % cationic lipid/neutral lipid, e.g., DSPC/Chol/PEG-modified lipid, e.g., PEG-DMG or PEG-cDMA) or 52/13/30/5 (mol % cationic lipid/neutral lipid, e.g., DSPC/Chol/PEG-modified lipid, e.g., PEG-DMG or PEG-cDMA).

The amino alcohol cationic lipid may be the lipids described in and/or made by the methods described in U.S. Patent Publication No. US20130150625, which is incorporated by reference in its entirety for all purposes. As a non-limiting example, the cationic lipid may be 2-amino-3-[(9Z,12Z)-octadeca-9,12-dien-1-yloxy]-2-{[(9Z,2Z)-octadeca-9,12-1-dien-1-yloxy]methyl}propan-1-ol (Compound 1 in US20130150625); 2-amino-3-[(9Z)-octadec-9-en-1-yloxy]-2-{[(9Z)-octadec-9-en-1-yloxy]methyl}propan-1-ol (Compound 2 in US20130150625); 2-amino-3-[(9Z,12Z)-octadeca-9,12-dien-1-yloxy]-2-[(octyloxy)methyl]propa-n-1-ol (Compound 3 in US20130150625); and 2-(dimethylamino)-3-[(9Z,12Z)-octadeca-9,12-dien-1-yloxy]-2-{[(9Z, 12Z)-octadeca-9,12-dien-1-yloxy]methyl}propan-1-ol (Compound 4 in US20130150625); or any pharmaceutically acceptable salt or stereoisomer thereof.

A lipid nanoparticle formulation can be composed of (i) at least one lipid selected from the group consisting of 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy) heptadecanedioate (L319); (ii) a neutral lipid selected from DSPC, DPPC, POPC, DOPE and SM; (iii) a sterol, e.g., cholesterol; and (iv) a PEG-lipid, e.g., PEG-DMG or PEG-cDMA, in a molar ratio of 20-60% cationic lipid:5-25% neutral lipid:25-55% sterol; 0.5-15% PEG-lipid.

Examples of lipid nanoparticle compositions and methods of making them are described, for example, in Cifuentes-Rius et al., (2021) Nature Nanotechnol. 16:37-46; Hou et al., (2021) Nature Rev. 6:1078-1094; Jang et al., (2021) Int. J. Med. Sci. 22:10009 (doi.org/10.3390/ijms221810009); Semple et al. (2010) Nat. Biotechnol. 28:172-176; Jayarama et al. (2012), Angew. Chem. Int. Ed., 51:8529-8533; and Maier et al. (2013) Molecular Therapy 21, 1570-1578 (each of which are incorporated by reference in their entirety for all purposes).

A lipid nanoparticle formulation can be influenced by the selection of the cationic lipid component, the degree of cationic lipid saturation, the nature of the PEGylation, ratio of all components and biophysical parameters such as size. For example, in Semple et al. (Nature Biotech. 2010 28:172-176), the lipid nanoparticle formulation is composed of 57.1% cationic lipid, 7.1% dipalmitoylphosphatidylcholine, 34.3% cholesterol, and 1.4% PEG-c-DMA. As another example, changing the composition of the cationic lipid can more effectively deliver siRNA to various antigen presenting cells (Basha et al. Mol Ther. 2011 19:2186-2200, which is incorporated by reference in its entirety for all purposes).

A kit can contain an anti-EPOR and/or an anti-CD131 antibody and/or an anti-EPO antibody and/or an EPO analog or a pharmaceutical composition comprising the same, and instructions for administering or using the anti-EPOR antibody and/or anti-CD131 antibody and/or anti-EPO antibody and/or EPO analog, or the pharmaceutical composition comprising the same to treat an antibody-associated condition.

In some aspects, provided herein is a cell comprising an anti-EPO antibody, an anti-EPOR antibody, or an anti-CD131 antibody, an EPO analog. In some embodiments, a cell can comprise an immune cell. Examples of an immune cell can include, but are not limited to, a macrophage, a dendritic cell, a T-cell, a natural killer cell, or a B cell. In some embodiments, a T-cell can comprise a cytotoxic T-cell. In some embodiments, a cell can comprise a myeloid cell. In some embodiments, a myeloid cell can comprise a granulocyte, a monocyte, a macrophage, or a dendritic cell. In some embodiments, a cell is an erythroid progenitor cell. In some embodiments, a cell can comprise an endothelial cell.

Uses of Anti-EPOR Antibodies, Anti-CD131 Antibodies, Anti-EPO Antibodies, and/or EPO Analogs

In some aspects, anti-EPOR antibodies, anti-CD131 antibodies, anti-EPO antibodies, and/or EPO analogs described herein can be used in a method of treating, preventing, and/or delaying progression of a tumor or a cancer in an individual, a patient, or a subject in need thereof. In some aspects, anti-EPOR antibodies, anti-CD131 antibodies, anti-EPO antibodies, and/or EPO analogs described herein can be used in a method of treating, preventing, inhibiting, and/or delaying progression of a metastatic cancer. In some embodiments, anti-EPOR antibodies, anti-CD131 antibodies, anti-EPO antibodies, and/or EPO analogs described herein can be used in a method of inhibiting cancer metastasis. In some embodiments, an individual, a patient, or a subject in need thereof may have an existing metastatic cancer. In some embodiments, an individual, a patient, or a subject in need thereof may not have an existing metastatic cancer. In some embodiments, the present disclosure relates to the use of anti-EPOR antibodies, anti-CD131 antibodies, anti-EPO antibodies, and/or EPO analogs described herein for the preparation of a medicament for the treatment, prevention, inhibition, and/or delay of progression of a cancer or a metastatic cancer.

In some embodiments, the method can comprise administering a therapeutically effective amount of any of anti-EPOR antibodies, anti-CD131 antibodies, anti-EPO Antibodies, and/or EPO analogs described herein or any pharmaceutical composition comprising any of anti-EPOR antibodies, anti-CD131 antibodies, anti-EPO Antibodies, and/or EPO analogs described herein to a an individual, a patient, or a subject in need thereof. In some embodiments, the individual, the patient, or the subject having the tumor or the cancer may be resistant to a checkpoint inhibitor therapy. In some embodiments, the individual, the patient, or the subject having the tumor or the cancer may have failed to be treated by a checkpoint inhibitor therapy. In some embodiments, the individual, the patient, or the subject having the tumor or the cancer may be responsive to a checkpoint inhibitor therapy. In some embodiments, the tumor or the cancer can comprise metastatic cancer.

In some embodiments, a therapeutically effective amount in the context of the administration of any composition or pharmaceutical composition comprising anti-EPOR antibodies, anti-CD131 antibodies, anti-EPO Antibodies, and/or EPO analogs described herein can refer to an amount of a composition or pharmaceutical composition to a subject in need thereof which has a therapeutic effect and/or beneficial effect. In certain specific embodiments, an effective amount in the context of the administration of composition, pharmaceutical composition, or medicament thereof to a patient can result in one, two or more of the following effects: (i) reduces or ameliorates the severity of a disease; (ii) inhibits or delays onset of a disease; (iii) inhibits the progression of a disease; (iv) increases the survival of a subject; (v) reduces the number of symptoms associated with a disease; (vi) reduces or ameliorates the severity of a symptom associated with a disease; (vii) reduces the duration of a symptom associated with a disease associated; (viii) prevents the recurrence of a disease or a symptom associated with a disease; (ix) inhibits or delays the development or onset of a symptom of a disease; and/or (x) inhibits of the progression of a symptom associated with a disease. In some embodiments, a disease can comprise a cancer, a metastatic cancer, or a combination thereof.

In some embodiments, a therapeutically effective amount of a composition, pharmaceutical composition, or medicament thereof is an amount effective to prevent or inhibit metastasis of a tumor or cancer in a subject having a tumor or cancer. In some embodiments, a therapeutically effective amount of a composition, pharmaceutical composition, or medicament thereof is an amount effective to inhibit formation of a pre-metastatic niche in a distant site that is different from a location of a primary tumor. In some embodiments, a therapeutically effective amount of a composition, pharmaceutical composition, or medicament thereof is an amount effective to inhibit a metastatic nodule growth. In some embodiments, a distant site can comprise a site that is different from a location of a primary tumor. In some embodiments, a distant site can comprise a tissue that is distinct from a tissue where a primary tumor is located. In some embodiments, a subject may have an existing metastatic cancer. In some embodiments, a subject may not have an existing metastatic cancer.

In one aspect, anti-EPOR antibodies, anti-CD131 antibodies, anti-EPO Antibodies, and/or EPO analogs described herein can be used to overcome immunosuppressive or tolerogenic states in a subject. For example, these anti-EPOR antibodies, anti-CD131 antibodies, anti-EPO Antibodies, and/or EPO analogs described herein can be used to overcome a tumor immune suppressive microenvironment, to boost immune response to vaccines, to enhance the immune response during an acute inflammatory response to disease (e.g., an infection from a microorganism or a virus), and/or to treat chronic infectious diseases or conditions. In some embodiments, anti-EPOR antibodies, anti-CD131 antibodies, anti-EPO Antibodies, and/or EPO analogs described herein can inhibit immune tolerance. In some embodiments, inhibiting immune tolerance can comprise promoting or increasing immune response. For example, inhibiting immune tolerance can comprise increasing immune response to a vaccine, a viral infection, a bacterial infection, or a tumor antigen (e.g., an antigen produced by cancer).

In some embodiments, anti-EPOR antibodies, anti-CD131 antibodies, anti-EPO Antibodies, and/or EPO analogs described herein can promote differentiation of naïve T cells into effector T cells. Markers for effector T cells described herein can include, but are not limited to, Cluster of Differentiation 45 (CD45), CD3, CD8, Perforin, Interferon gamma (IFNγ), Granzyme B, or tumor necrosis factor alpha (TNFα). In some embodiments, anti-EPOR antibodies, anti-CD131 antibodies, anti-EPO Antibodies, and/or EPO analogs described herein can inhibit differentiation of naïve T cells into regulatory T cells. Markers for regulatory T cells described herein can include, but are not limited to, Cluster of Differentiation 4 (CD4), CD25, CD127, Forkhead Box P3 (FoxP3), CD39, protein tyrosine phosphatase receptor type C (CD45RA), Interleukin-2 (IL-2), or a Cytotoxic T-Lymphocyte Associated Protein 4 (CTLA-4). In some embodiments, anti-EPOR antibodies, anti-CD131 antibodies, anti-EPO Antibodies, and/or EPO analogs described herein can increase a number of progenitor exhausted T cells. Markers for progenitor exhausted T cells can include, but are not limited to, Cluster of Differentiation 44 (CD44), Signaling lymphocyte activation molecule family member 6 (SLAMF6) or T cell factor 1 (TCF1).

In some embodiments, anti-EPOR antibodies, anti-CD131 antibodies, anti-EPO Antibodies, and/or EPO analogs described herein can stimulate immune response in cancer. For example, anti-EPOR antibodies, anti-CD131 antibodies, anti-EPO Antibodies, and/or EPO analogs described herein can render cancer cells sensitive to an immune checkpoint inhibitor. Examples of immune checkpoint inhibitors can include, but are not limited, to PD-1 inhibitors, PD-L1 inhibitors, and/or CTLA-4 inhibitors. In some embodiments, immune checkpoint inhibitors can comprise anti-CTLA-4 antibodies, anti-PD-1 antibodies, anti-PD-L1 antibodies, or functional fragments thereof, or combinations thereof. In some embodiments, immune checkpoint inhibitors can comprise Nivolumab, Pembrolizumab, Cemiplimab, Atezolimumab, Durvalumab, Avelumab, or Ipilimumab. In some embodiments, anti-EPOR antibodies, anti-CD131 antibodies, anti-EPO Antibodies, and/or EPO analogs described herein can attenuate tumor growth. In some embodiments, anti-EPOR antibodies, anti-CD131 antibodies, anti-EPO Antibodies, and/or EPO analogs described herein can reduce the size of a cancer or attenuate the growth of a cancer.

Tumors are frequently infiltrated with myeloid cells with immune tolerogenic or suppressive functions. Examples of myeloid cells include, but are not limited to, granulocytes, monocytes, macrophages (MΦs), or dendritic cells (DCs). The hetero-EPOR is widely present and upregulated in such tumor-infiltrating myeloid cells including both dendritic cells (DCs) and macrophages (MΦs), and contributes to immune tolerance or suppression. An antagonistic anti-EPOR antibodies, anti-CD131 antibodies, anti-EPO Antibodies, and/or EPO analogs described herein can block the activation of the hetero-EPOR (e.g., on myeloid cells) and can prevent, inhibit, or reduce immune suppression and antigen-specific immune tolerance thereby enabling effective anti-tumor immunity. The ability of such blocking antibodies to reverse hetero-EPOR mediated immune tolerance can be validated in a variety of cancer models, e.g., liver hepatocarcinoma, colorectal cancer, breast cancer, brain cancer, liver metastasis, and lymph node metastasis etc. In addition to cancers, in some embodiments, anti-EPOR antibodies, anti-CD131 antibodies, anti-EPO Antibodies, and/or EPO analogs described herein can be used to treat chronic infections. For example, chronic viral infections (e.g., Hepatitis B Virus, Herpes Simplex Virus, Human Papilloma Virus, Covid-19, influenza, Human Immunodeficiency Virus, meningitis, pneumonia, rotavirus, chicken pox, etc.) and/or chronic bacterial infections (e.g., Mycobacterium tuberculosis, fungal, anthrax, tetanus, leptospirosis, cholera, botulism, pseudomonas, pneumonia, E. Coli, gonorrhea, bubonic plague, syphilis, methicillin-resistant Staphylococcus aureus, meningitis, etc.) can be treated similarly. These anti-EPOR antibodies, anti-CD131 antibodies, anti-EPO Antibodies, and/or EPO analogs described herein can also be used to reduce an immune tolerogenic and/or immunosuppressive state for T-cells (e.g., cytotoxic T-cells, CAR T-cells, or TCR engineered T-cells) or natural killer cells (e.g., NK cells engineered with CARs or T-cell receptors).

In some embodiments, cancer may be associated with immunosuppressive cells. In some embodiments, cancer may be associated with immunosuppressive myeloid cells. In some embodiments, cancer may be associated with immunosuppressive T cells. In some embodiments, cancer may be associated with effector T cells. In some embodiments, cancer may be associated with tumor-specific effector T cells.

Neoplasia, tumors and cancers that can be treated with the analogs and antibodies described herein can include, for example, benign, malignant, metastatic and non-metastatic types, and can include any stage (I, II, III, IV or V) or grade (G1, G2, G3, etc.) of neoplasia, tumor, or cancer, or a neoplasia, tumor, cancer or metastasis that is progressing, worsening, stabilized or in remission. Cancers that may be treated using the compositions and methods provided herein can include, but are not limited to, cells or neoplasms of the bladder, blood, bone, bone marrow, brain, breast, colon, esophagus, gastrointestines, gum, head, kidney, liver, lung, nasopharynx, neck, ovary, prostate, skin, stomach, testis, tongue, or uterus. In addition, the cancer may specifically be of the following histological type, though it is not limited to the following: neoplasm, malignant; carcinoma; undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; bronchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; Paget's disease, mammary; acinar cell carcinoma; adenosquamous carcinoma; adenocarcinoma with squamous metaplasia; thymoma, malignant; ovarian stromal tumor, malignant; thecoma, malignant; granulosa cell tumor, malignant; androblastoma, malignant; Sertoli cell carcinoma; Leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malignant melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor; Mullerian mixed tumor; nephroblastoma; hepatoblastoma; carcinosarcoma; mesenchymoma, malignant; Brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; Kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; Ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; Hodgkin's disease; Hodgkin's; paragranuloma; malignant lymphoma, small lymphocytic; malignant lymphoma, large cell, diffuse; malignant lymphoma, follicular; mycosis fungoides; other specified non-Hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia. In some embodiments, the neoplastic disease may be tumors associated with a cancer selected from prostate cancer, liver cancer, renal cancer, lung cancer, breast cancer, colorectal cancer, pancreatic cancer, brain cancer, hepatocellular cancer, lymphoma, leukemia, gastric cancer, cervical cancer, ovarian cancer, thyroid cancer, melanoma, head and neck cancer, skin cancer and soft tissue sarcoma and/or other forms of carcinoma. The tumor may be metastatic or a malignant tumor.

In some embodiments, anti-EPOR antibodies, anti-CD131 antibodies, anti-EPO Antibodies, and/or EPO analogs described herein can be used to promote vaccination-associated antigen-specific immunity. Effective vaccination can be challenging for a number of pathological conditions. Blocking hetero-EPOR signaling in the presence of specific antigen(s) can be effective at promoting antigen-specific immunity. This can be achieved by targeting hetero-EPOR expressing dendritic cells with the antigen and the above-mentioned antagonistic EPO analogs, antagonistic anti-hetero-EPOR antibodies, and/or anti-EPO antibodies that inhibit EPO from interacting with hetero-EPOR to enhance the immune response. It can also be achieved by nanoparticles that encapsulate mRNA of the antigen and an inhibitor of the hetero-EPOR signaling pathway which acts either on the heterodimeric receptor or its downstream intracellular signaling pathway. Exemplary vaccines can include vaccines for HIV, HCV, HSV, HBV, cancer vaccines, and/or virally caused diseases requiring repeated injections and/or immunity is short-lived, e.g., HBV, COVID, Influenza A, and/or Shingles.

In some aspects, EPO analogs that are agonists for the homo-EPOR and do not bind or are antagonists of the hetero-EPOR, and/or anti-EPO antibodies that inhibit binding of EPO to the hetero-EPOR and/or anti-hetero-EPOR antibodies that are antagonists for the hetero-EPOR can be used with or without erythropoietin-stimulating agents (ESA) for cancer patients in need to an ESA treatment. In this aspect, any cancer patient needing an ESA can be provided the ESA combined with these EPO analogs, and/or anti-EPOR antibodies, and/or anti-EPO antibodies.

In some embodiments, the use of ESAs in cancer patients can be limited because of the risk of thromboembolic events and accelerated disease progression and shortened survival. In this embodiment, immune tolerance and/or suppression mediated by activation of the hetero-EPOR on tumor infiltrated myeloid cells including both dendritic cells (DCs) and macrophages (MΦs) can be a major contributor to the enhanced tumor growth and shortened survival seen in cancer patients treated with ESA. In this embodiment, a non-immune tolerogenic or non-suppressive ESA can activate the homo-EPOR and not the hetero-EPOR and can be used to treat anemia in cancer patients without promoting immune tolerance or suppression. Since the interaction site between EPO and the hetero-EPOR resides in helix B of EPO, and helix B is not involved in binding to the homo-EPOR, EPO analogs with changes in helix B that inhibit binding to the hetero-EPOR may not interfere with binding to the homo-EPOR, resulting in analogs with the desired receptor activity profile for this use of ESAs in cancer patients. Alternatively, an anti-EPO antibody that neutralizes (or inhibits) binding to the hetero-EPOR while not interfering with EPO binding to the homo-EPOR can be combined with EPO (or other potential ESAs) to provide a combination that has the desired profile of activities at the hetero-EPOR and homo-EPOR for treatment of anemia in cancer patients.

The therapeutically effective amount and the frequency of administration of, and the length of treatment with EPO analogs anti-EPOR antibodies, anti-CD131 antibodies, and/or anti-EPO antibodies disclosed herein to treat an antibody-associated condition may depend on various factors, including the nature and severity of the condition, the potency of the antibody, the mode of administration, the age, body weight, general health, gender and diet of the subject, and the response of the subject to the treatment, and can be determined by the treating physician. The therapeutically effective amount of the antibody and/or analog can be from about 1, 5 or 10 mg to about 200 mg, from about 1, 5 or 10 mg to about 150 mg, from about 1, 5 or 10 mg to about 100 mg, or from about 1, 5 or 10 mg to about 50 mg, or as deemed appropriate by the treating physician, which can be administered in a single dose or in divided doses. The therapeutically effective amount of the antibody and/or analog can be about 1-5 mg, about 5-10 mg, about 10-20 mg, about 20-30 mg, about 30-40 mg, about 40-50 mg, about 50-100 mg, about 100-150 mg, or about 150-200 mg. The therapeutically effective amount of the antibody and/or analog can be about 1 mg, about 5 mg, about 10 mg, about 15 mg, about 20 mg, about 25 mg, about 30 mg, about 40 mg, about 50 mg, about 60 mg, about 70 mg, about 80 mg, about 90 mg, about 100 mg, about 150 mg, or about 200 mg. The therapeutically effective amount of the antibody and/or analog can be about 1-5 mg, about 5-10 mg, or about 10-50 mg. The therapeutically effective amount of the antibody and/or analog can be about 0.01-0.1 mg/kg, about 0.1-0.5 mg/kg, about 0.5-1 mg/kg, about 1-2 mg/kg, about 2-3 mg/kg, about 3-5 mg/kg, about 5-10 mg/kg, or about 10-15 mg/kg body weight, or as deemed appropriate by the treating physician. The therapeutically effective amount of the antibody and/or analog can be about 0.01-0.1 mg/kg, about 0.1-0.5 mg/kg, about 0.5-1 mg/kg, about 1-3 mg/kg, about 3-5 mg/kg, about 5-10 mg/kg, or about 10-15 mg/kg body weight.

In some aspects, an antibody and/or analog can be administered in any suitable frequency to treat a patient. The antibody or analog can be administered once daily, once every 2 days, once every 3 days, twice weekly, once weekly, once every 2 weeks, once every 3 weeks, once monthly, once every 6 weeks, once every 2 months, or once every 3 months, or as deemed appropriate by the treating physician. The antibody and/or analog can be administered once weekly or once every 2 weeks.

Likewise, an antibody and/or analog can be administered for any suitable length of time, or in any suitable total number of doses, to treat a patient. The antibody and/or analog is administered over a period of at least about 1 week, 2 weeks, 3 weeks, 1 month (4 weeks), 6 weeks, 2 months, 3 months, 6 months, 1 year, 2 years, 3 years or longer, or as deemed appropriate by the treating physician. The condition treated can be a chronic condition. A chronic condition can exist for, e.g., at least about 6 weeks or 2 months or longer. The antibody and/or analog can be administered over a period of at least about 6 weeks, about 2 months, about 3 months, or about 6 months. In some embodiments, 1, 2, 3, 4, 5, or 6 doses of the antibody and/or analog can be administered for the entire treatment regimen. In some embodiments, 1, 2, or 3 doses of the antibody and/or analog can be administered for the entire treatment regimen.

In some aspects, an antibody and/or analog can also be administered in an irregular manner to treat a patient. For example, the antibody and/or analog can be administered 1, 2, 3, 4, 5, or more times in a period of 1 week, 2 weeks, 3 weeks, 1 month, 2 months, or 3 months in an irregular manner. Furthermore, an antibody and/or analog can be taken pro re nata (as needed) for treatment of a patient. For instance, the antibody and/or analog can be administered 1, 2, 3, 4, 5, or more times, whether in a regular or irregular manner, for treatment of a patient. The appropriate dosage of, frequency of dosing of and length of treatment with the antibody and/or analog can be determined by the treating physician.

For a more rapid establishment of a therapeutic level of an antibody or analog at least one loading dose of the antibody and/or analog can be administered prior to the maintenance dose. A loading dose can be administered, followed by (i) one or more additional loading doses and then one or more therapeutically effective maintenance doses, or (ii) one or more therapeutically effective maintenance doses without an additional loading dose, as deemed appropriate by the treating physician. A loading dose of an antibody and/or analog can be larger (e.g., about 1.5, 2, 3, 4, or 5 times larger) than a subsequent maintenance dose and is designed to establish a therapeutic level of the drug more quickly. The one or more therapeutically effective maintenance doses can be any therapeutically effective amount described herein. The loading dose can be about 2 or 3 times larger than the maintenance dose. A loading dose can be administered on day 1, and a maintenance dose can be administered, e.g., once weekly or once every 2 weeks thereafter for the duration of treatment. The antibody and/or analog can be administered in a loading dose of about 2-10 mg, about 10-20 mg, or about 20-100 mg, or about 3-15 mg, about 15-30 mg, or about 30-150 mg, on day 1, followed by a maintenance dose of about 1-5 mg, about 5-10 mg, or about 10-50 mg once weekly or once every 2 weeks for the duration of treatment (e.g., for at least about 2, 3, or 6 months), where the loading dose is about 2 or 3 times larger than the maintenance dose and the antibody or analog is administered parenterally (e.g., intravenously, subcutaneously or intramuscularly).

In some embodiments, two (or more) loading doses of the antibody and/or analog can be administered prior to the maintenance dose. A first loading dose of the antibody and/or analog can be administered on day 1, a second loading dose can be administered, e.g., about 1 or 2 weeks later, and a maintenance dose can be administered, e.g., once weekly or once every 2 weeks thereafter for the duration of treatment. The first loading dose can be about 3 or 4 times larger than the maintenance dose, and the second loading dose can be about 2 times larger than the maintenance dose. The antibody and/or analog can be administered in a first loading dose of about 3-15 mg, about 15-30 mg, or about 30-150 mg, or about 4-20 mg, about 20-40 mg, or about 40-200 mg, on day 1, in a second loading dose of about 2-10 mg, about 10-20 mg, or about 20-100 mg about 1 or 2 weeks later, followed by a maintenance dose of about 1-5 mg, about 5-10 mg, or about 10-50 mg once weekly or once every 2 weeks for the duration of treatment (e.g., for at least about 2, 3 or 6 months), where the first loading dose can be about 3 or 4 times larger than the maintenance dose, the second loading dose can be about 2 times larger than the maintenance dose, and the antibody or analog can be administered parenterally (e.g., intravenously, subcutaneously or intramuscularly).

Combination Therapies with Additional Therapeutic Agents

The disclosure provides a method of treating a patient, comprising administering to a subject in need of treatment a therapeutically effective amount of an antibody and/or analog described herein, optionally in combination with an additional therapeutic agent. The disclosure further provides an antibody and/or analog described herein, or a composition comprising an antibody and/or analog described herein, for use as a medicament, optionally in combination with an additional therapeutic agent. In addition, the disclosure provides for the use of an antibody and/or analog described herein in the preparation of a medicament, optionally in combination with an additional therapeutic agent.

One or more additional therapeutic agents can optionally be used in combination with an antibody or analog to treat a patient. The optional additional therapeutic agent(s) can be administered to a subject concurrently with (e.g., in the same composition as the antibody and/or analog or in separate compositions) or sequentially to (before or after) administration of the antibody and/or analog. The optional additional therapeutic agent(s) can be selected from anti-cancer agents, immunotherapy agents, immunosuppressive agents, anti-inflammatory agents, allergy drugs, and combinations thereof. One or more immunosuppressive agents can be used in combination with an antibody and/or analog to treat a patient.

Anti-cancer agents can include, for example, a chemotherapeutic, an antibody, an antibody-drug conjugate, an immunotherapy, a chimeric antigen receptor cell therapy, a radiotherapy, an alkylating agent, a plant alkaloid, an antitumor antibiotic, an antimetabolite, a topoisomerase inhibitor, and/or an anti-neoplastic.

Antibodies and antibody-drug conjugates (ADC) can bind to a tumor associated antigen. The drug component of the ADC can be, for example, a chemotherapeutic, a radionucleotide, an alkylating agent, a plant alkaloid, an antitumor antibiotic, an antimetabolite, a topoisomerase inhibitor, and/or an anti-neoplastic. The drug component of the ADC can be attached to the antibody through a linker which can be cleavable or non-cleavable in nature.

Alkylating agents can include, for example, mustard gas derivatives (e.g., mechlorethamine, cyclophosphamide, chlorambucil, melphalan, or ifosfamide), ethylenimines (e.g., thiotepa or hexamethylmelamine), alkylsulfonates (e.g., busulfan), hydrazines and triazines (e.g., altretamine, procarbazine, dacarbazine, or temozolomide), nitrosoureas (e.g., carmustine, lomustine or streptozocin), and metal salts (e.g., carboplatin, cisplatin, or oxaliplatin). Plant alkaloids can include, for example, Vinca alkaloids (e.g., vincristine, vinblastine, or vinorelbine), taxanes (e.g., paclitaxel or docetaxel), podophyllotoxins (e.g., etoposide or tenisopide), and camptothecan analogs (e.g., irinotecan or topotecan). Antitumor antibiotics can include, for example, anthracyclines (e.g., doxorubicin, daunorubicin, epirubicin, mixoantrone, or idarubicin), and chromomycins (e.g., dactinomycin or plicamycin). Antimetabolites can include, for example, folic acid antagonists (e.g., methotrexate), pyrimidine antagonists (e.g., 5-flurouracil, foxuridine, cytarabine, capecitabine, or gemcitabine), purine antagonists (e.g., 6-mercaptopurine or 6-thioguanine), and adenosine deaminase inhibitors (e.g., cladribine, fludarabine, nelarabine, or pentostatin). Topoisomerase inhibitors can include, for example, topoisomerase I inhibitors (e.g., irinotecan or topotecan) and topoisomerase II inhibitors (e.g., amsacrine, etoposide, etoposide phosphate, or teniposide). Anti-neoplastics can include, for example, ribonucleotide reductase inhibitors (e.g., hydroxyurea), adrenocortical steroid inhibitors (e.g., mitotane), enzymes (e.g., asparaginase or pegaspargase), antimicrotubule agents (e.g., estramustine), and retinoids (e.g., bexarotene, isotretinoin, or tretinoin).

Other chemotherapeutic drugs can include, for example, an anthracycline, a camptothecin, a tubulin inhibitor, a maytansinoid, a calicheamycin, a pyrrolobenzodiazepine dimer (PBD), an auristatin, a nitrogen mustard, an ethylenimine derivative, an alkyl sulfonate, a nitrosourea, a triazene, a folic acid analog, a taxane, a COX-2 inhibitor, a pyrimidine analog, a purine analog, an antibiotic, an enzyme inhibitor, an epipodophyllotoxin, a platinum coordination complex, a vinca alkaloid, a substituted urea, a methyl hydrazine derivative, an adrenocortical suppressant, a hormone antagonist, an antimetabolite, an alkylating agent, an antimitotic, an anti-angiogenic agent, a tyrosine kinase inhibitor, an mTOR inhibitor, a heat shock protein (HSP90) inhibitor, a proteosome inhibitor, an HDAC inhibitor, a pro-apoptotic agent, and a combination thereof.

Other chemotherapeutic agents can include, for example, 5-fluorouracil, afatinib, aplidin, azaribine, anastrozole, anthracyclines, axitinib, AVL-101, AVL-291, bendamustine, bleomycin, bortezomib, bosutinib, bryostatin-1, busulfan, calicheamycin, camptothecin, carboplatin, 10-hydroxycamptothecin, carmustine, celecoxib, chlorambucil, cisplatinum, COX-2 inhibitors, irinotecan (CPT-11), SN-38, carboplatin, cladribine, camptothecans, crizotinib, cyclophosphamide, cytarabine, dacarbazine, dasatinib, dinaciclib, docetaxel, dactinomycin, daunorubicin, DM1, DM3, DM4, doxorubicin, 2-pyrrolinodoxorubicine (2-PDox), a pro-drug form of 2-PDox (pro-2-PDox), cyano-morpholino doxorubicin, doxorubicin glucuronide, endostatin, epirubicin glucuronide, erlotinib, estramustine, epidophyllotoxin, erlotinib, entinostat, estrogen receptor binding agents, etoposide (VP16), etoposide glucuronide, etoposide phosphate, exemestane, fingolimod, floxuridine (FUdR), 3′,5′-o-dioleoyl-FudR (FUdR-dO), fludarabine, flutamide, farnesyl-protein transferase inhibitors, flavopiridol, fostamatinib, ganetespib, GDC-0834, GS-1101, gefitinib, gemcitabine, hydroxyurea, ibrutinib, idarubicin, idelalisib, ifosfamide, imatinib, lapatinib, lenolidamide, leucovorin, LFM-A13, lomustine, mechlorethamine, melphalan, mercaptopurine, 6-mercaptopurine, methotrexate, mitoxantrone, mithramycin, mitomycin, mitotane, monomethylauristatin F (MMAF), monomethylauristatin D (MMAD), monomethylauristatin E (MMAE), navelbine, neratinib, nilotinib, nitrosurea, olaparib, plicomycin, procarbazine, paclitaxel, PCI-32765, pentostatin, PSI-341, raloxifene, semustine, SN-38, sorafenib, streptozocin, SU11248, sunitinib, tamoxifen, temazolomide, transplatinum, thalidomide, thioguanine, thiotepa, teniposide, topotecan, uracil mustard, vatalanib, vinorelbine, vinblastine, vincristine, vinca alkaloids and ZD1839. In some embodiments, the chemotherapeutic agents can be SN-38.

Immunotherapy is directed at boosting the body's natural defenses in order to fight a disease, a cancer or tumor. It capitalizes on the substances made by the body, or artificially in a laboratory, to improve or restore immune system function. Immunotherapies can include checkpoint inhibitors that target immune checkpoints such as CTLA-4 and PD-1/PD-L1, key regulators of the immune system that dampen the immune response. Immunotherapies can comprise anti-CTLA-4 antibody, an anti-PD-1 antibody, an anti-PD-L1 antibody, or any combinations thereof. Examples of checkpoint inhibitors that may be used as payloads can include, for example, Nivolumab (Opdivo®), Pembrolizumab (Keytruda®), Cemiplimab (Libtayo®), Atezolizumab (Tecentriq®), Avelumab (Bavencio®), Durvalumab (Imfinzi®), Ipilimumab (Yervoy®), Lirilumab, and BMS-986016. Nivolumab, Atezolizumab and Pembrolizumab can act at the checkpoint protein PD-1 and can inhibit apoptosis of anti-tumor immune cells. Some checkpoint inhibitors can prevent the interaction between PD-1 and its ligand PD-L1. Ipilimumab can act at CTLA-4 and can prevent CTLA-4 from downregulating activated T-cells in the tumor. Lirilumab can act at KIR and can facilitate activation of Natural Killer cells. BMS-986016 can act at LAG3 and can activate antigen-specific T-lymphocytes and can enhance cytotoxic T cell-mediated lysis of tumor cells. Other types of immunotherapies can include, for example, monoclonal antibodies, tumor-agnostic therapies, non-specific immunotherapies, oncolytic virus therapy, adoptive cell transfer, e.g., CAR T-cell therapy and cancer vaccines. Non-specific immunotherapies can include treatment with interferons or interleukins, molecules which can help the immune system fight cancer and either slow the growth of cancer cells or, in some instance, destroy the cancer. Immunotherapies may be given instead of traditional cancer treatments, such as chemotherapy or radiation therapy, or in combination with such treatments.

Adoptive cell therapy may use cells that have originated from the subject (autologous) or from another subject (allogeneic). Examples of such adoptive cell therapies can include, but are not limited to, engineered or non-engineered macrophages, engineered or non-engineered T-cells, and/or engineered or non-engineered natural killer cells. Accordingly, adoptive cell therapies can include tumor-Infiltrating Lymphocyte (TIL) therapy, Engineered T Cell Receptor (TCR) therapy, and/or natural killer (NK) cell therapy, the details of which will be well known to those skilled in the art (Adoptive cellular therapies: the current landscape, Rohaan et al. 2019, Virchows Arch. 474(4): 449-461, which is incorporated by reference in its entirety for all purposes).

Immunosuppressive agents can include, for example, anti-CD20 antibodies (e.g., rituximab), calcineurin inhibitors (e.g., tacrolimus, cyclosporine, etc.), antiproliferative agents or IDMH inhibitors (e.g., mycophenolate mofetil, mycophenolate sodium, azathioprine, leflunomide, etc.), mTOR inhibitors (e.g., Sirolimus, everolimus, etc.), steroids (e.g., corticosteroids such as prednisone, budesonide, prednisolone, etc.), and biologics (e.g., abatacept, adalimumab, anakinra, certolizumab, etanercept, infliximab, ixekizumab, natalizumab, rituximab, secukinumab, tocilizumab, uestekinumab, vedolizumab, basiliximab, daclizumab, muromonab). Biologics can also include, for example, CTLA 4 fusion proteins, anti-TNFα antibodies, IL-1 receptor antagonist protein, TNF receptor fusion proteins, anti-IL17A antibodies, anti-α₄β₇ integrin antibodies, anti-IL6 receptor antibodies, anti-p40 subunit of IL12/IL23 antibodies, anti-dα₄β₇ integrin antibodies, anti-CD25 antibodies, and anti-CD3 antibodies.

One or more anti-inflammatory agents can be used in combination with an antibody or analog to treat a patient. The one or more anti-inflammatory agents can include, for example, an inhibitor of a pro-inflammatory cytokine or a receptor therefor or the production thereof (e.g., TNF-α or/and IL-6 or IL-6R). Other anti-inflammatory agents can include, for example: non-steroidal anti-inflammatory drugs (NSAIDs), immunomodulators, immunosuppressants, anti-inflammatory cytokines and compounds that increase their production, inhibitors of pro-inflammatory cytokines or receptors therefor, inhibitors of the production of pro-inflammatory cytokines or receptors therefor, inhibitors of pro-inflammatory transcription factors or their activation or expression, inhibitors of pro-inflammatory prostaglandins (e.g., prostaglandin E₂ [PGE₂]) or receptors therefor (e.g., EP₃) or the production thereof, inhibitors of leukotrienes or receptors therefor or the production thereof, inhibitors of phospholipase A2 (e.g., secreted and cytosolic PLA2), suppressors of C-reactive protein (CRP) activity or level, mast cell stabilizers, phosphodiesterase inhibitors, specialized pro-resolving mediators (SPMs), other kinds of anti-inflammatory agents, and analogs, derivatives, fragments and salts thereof.

Non-steroidal anti-inflammatory drugs (NSAIDs) can include, but are not limited to, acetic acid derivatives, anthranilic acid derivatives (fenamates), enolic acid derivatives (oxicams), propionic acid derivatives, salicylates, COX-2-selective inhibitors, other kinds of NSAIDs, such as monoterpenoids (e.g., eucalyptol and phenols [e.g., carvacrol]), anilinopyridinecarboxylic acids (e.g., clonixin), sulfonanilides (e.g., nimesulide), and dual inhibitors of lipooxygenase (e.g., 5-LOX) and cyclooxygenase (e.g., COX-2) (e.g., chebulagic acid, licofelone, 2-(3,4,5-trimethoxyphenyl)-4-(N-methylindol-3-yl)thiophene, and di-tert-butylphenol-based compounds [e.g., DTPBHZ, DTPINH, DTPNHZ and DTPSAL]); and analogs, derivatives and salts thereof.

The glucocorticoid class of corticosteroids can have anti-inflammatory and immunosuppressive properties. Glucocorticoids can include, but are not limited to, hydrocortisone types, halogenated steroids, carbonates, and analogs, derivatives and salts thereof.

The optional additional therapeutic agent(s) independently can be administered in any suitable mode. Potential modes of administration can include, but are not limited to, oral, parenteral (including intradermal, subcutaneous, intramuscular, intravascular, intravenous, intraarterial, intraperitoneal, intramedullary, intrathecal and topical), intracavitary, and topical (including dermal/epicutaneous, transdermal, mucosal, transmucosal, intranasal [e.g., by nasal spray or drop], intraocular [e.g., by eye drop], pulmonary [e.g., by oral or nasal inhalation], buccal, sublingual, rectal [e.g., by suppository] and vaginal [e.g., by suppository]). In some embodiments, the optional additional therapeutic agent(s) independently can be administered orally or parenterally (e.g., intravenously, subcutaneously or intramuscularly).

One or more anti-allergy agents can be used in combination with an antibody or analog to treat a patient. Such anti-allergy agents can include, for example, antihistamines (e.g., cetirizine, fexofenadine, levocetirizine, loratidine, bormpheniramine, chlorpheniramine, celmastine, diphenhydramine, ketotifen, naphazoline, pheniramine, desloratadine, azelastine, epinastine, olopatadine), decongestants (e.g., pseudoephedrine, phenylephrine, oxymetazoline), steroids (e.g., beclomethasone, ciclesonide, fluticasone furoate, mometasone, budesonide, triamcinolone, dexamethasone, loteprednol, prednisone epocrates), mast cell stabilizers (e.g., cromolyn sodium, lodoxamide-tromethamine, nedocromil, pemirolast), and leukotriene modifiers (e.g., monteleukast).

The optional additional therapeutic agent(s) independently can be administered in any suitable frequency, including, but not limited to, daily (1, 2 or more times per day), every two or three days, twice weekly, once weekly, every two weeks, every three weeks, monthly, every two months or every three months, or in an irregular manner or on an as-needed basis. The dosing frequency can depend on, e.g., the mode of administration chosen. The length of treatment with the optional additional therapeutic agent(s) can be determined by the treating physician and can independently be, e.g., at least about 1 day, 2 days, 3 days, 4 days, 1 week, 2 weeks, 3 weeks, 4 weeks (1 month), 6 weeks, 2 months, 3 months, 6 months, 1 year, 2 years, 3 years or longer.

### Production of EPO Related Antibodies

The disclosure provides polynucleotides comprising nucleic acid sequences that encode EPO related antibodies (e.g., anti-EPO antibodies, anti-EPOR antibodies, or anti-CD131 antibodies), and/or EPO analogs described herein. A polynucleotide can comprise a nucleic acid sequence that encodes an EPO analog or the VH domain or/and the VL domain of an anti-EPOR, an anti-CD131, or an anti-EPO mAb. A polynucleotide can comprise a nucleic acid sequence that encodes the EPO analog, or heavy chain or/and the light chain of an EPO related mAb (e.g., anti-EPO antibodies, anti-EPOR antibodies, or anti-CD131 antibodies). In some embodiments, nucleic acid sequences that encode EPO related antibodies (e.g., anti-EPO antibodies, anti-EPOR antibodies, or anti-CD131 antibodies), and/or EPO analogs described herein can be codon-optimized. In general, codon optimization can refer to a process of modifying a nucleic acid sequence for expression in a host cell of interest by replacing at least one codon (e.g., more than 1, 2, 3, 4, 5, 10, 15, 20, 25, 50, or more codons) of a native sequence with codons that are more frequently or most frequently used in the genes of that host cell while maintaining the native amino acid sequence. Codon usage tables are readily available, for example, at the “Codon Usage Database,” and these tables can be adapted in a number of ways. Computer algorithms for codon optimizing a particular sequence for expression in a particular host cell are also available, such as Gene Forge® (Aptagen, PA) and GeneOptimizer® (ThermoFischer, MA) which is preferred. In some embodiments, nucleic acid sequences that encode EPO related antibodies (e.g., anti-EPO antibodies, anti-EPOR antibodies, or anti-CD131 antibodies), and/or EPO analogs described herein may not be codon-optimized.

The disclosure further provides constructs (which may also be called expression or cloning constructs) comprising nucleic acid sequences that encode EPO related antibodies described herein. Suitable constructs include, but are not limited to, plasmids, cosmids, bacterial artificial chromosomes, yeast artificial chromosomes, lambda phages (e.g., those with lysogeny genes deleted), and viruses. A construct can be present in a cell episomally or integrated into a chromosome (either way the construct remains and is still a construct, a plasmid and/or a vector).

Various construct systems can be employed. One class of constructs utilize DNA elements derived from animal viruses such as adenovirus, baculovirus, bovine papilloma virus, polyoma virus, SV40 virus, vaccinia virus, and retroviruses (e.g., MMTV, MOMLV and rous sarcoma virus). Another class of constructs utilize RNA elements derived from RNA viruses such as eastern equine encephalitis virus, flaviviruses, and Semliki Forest virus.

A construct can comprise various other elements for optimal expression of mRNA in addition to a nucleic acid sequence that encodes, e.g., the VH domain or/and the VL domain, or the heavy chain or/and the light chain, of an EPO related mAb, or EPO analog. For example, a construct can contain a transcriptional promoter, a promoter plus an operator, an enhancer, an open reading frame with or without intron(s) or/and exon(s), a termination signal, a splice signal, a secretion signal sequence or a selectable marker (e.g., a gene conferring resistance to an antibiotic or cytotoxic agent), or any combination or all thereof.

The disclosure also provides host cells comprising or expressing constructs that encode EPO related antibodies or EPO analog described herein. Suitable host cells include, but are not limited to, eukaryotic cells, mammalian cells (e.g., BHK, CHO, COS, HEK293, HeLa, MDCKII and Vero cells), insect cells (e.g., Sf9 cells), yeast cells and bacterial cells (e.g., E. coli cells). The host cell can be a mammalian cell (e.g., a CHO cell or a HEK293 cell).

A host cell can comprise or express a construct that encodes the V_{H} domain or the V_{L} domain, or the heavy chain or the light chain, of an EPO related mAb or EPO analog. A host cell can comprise or express a single construct that encodes the EPO analog, or the V_{H} domain and the V_{L} domain, or the heavy chain and the light chain, of an EPO related mAb. The same host cell or separate host cells can comprise or express a construct that encodes the V_{H} domain or the heavy chain of an EPO related mAb, and a separate construct that encodes the V_{L} domain or the light chain of the mAb.

A construct can be transfected or introduced into a host cell by any method known in the art. Transfection agents and methods include without limitation calcium phosphate, cationic polymers (e.g., DEAE-dextran and polyethylenimine), dendrimers, fugene, cationic liposomes, electroporation, sonoporation, cell squeezing, gene gun, viral transfection and retroviral transduction.

Methods and conditions for culturing transfected host cells and recovering the recombinantly produced EPO related antibodies or EPO analog are known in the art, and may be varied or optimized depending on, e.g., the particular expression vector or/and host cell employed. The V_{H} domain or/and the V_{L} domain, or the heavy chain or/and the light chain, of an EPO related mAb can be recombinantly produced. The heavy chain and the light chain of an EPO related antibody whole IgG1, IgG2 or IgG4, or the heavy chain and the light chain of an EPO related Fab fragment optionally fused with a protracting moiety, are recombinantly produced.

### EXAMPLES

### Example 1—Immunoregulatory Macrophages can be Reprogrammed into Co-Stimulatory Macrophages by Restricting EPO-EPOR Signaling for Treating and Preventing Liver Metastasis

The novel cell lines derived from a spontaneous PDAC model to thoroughly investigate liver metastasis will be investigated. The majority of PDAC patients present with liver metastases at the time of diagnosis, and these patients have the poorest survival rates. Current liver metastasis models using transplantation fail to capture the early metastatic expansion from single cells or small clusters, as well as primary tumor invasion and intravasation seen in human PDAC. While some autochthonous mouse models can recapitulate the entire carcinogenic process, high variability in tumor initiation and metastasis latency, coupled with the technical difficulties of crossbreeding for immunological studies, makes these models unfeasible for studying the processes responsible for liver metastasis, especially in the context of effects of and on the immune system and inflammation. To address this, liver-metastatic cell lines derived from electroporation-induced PDAC tumors are established. When orthotopically injected into the pancreas, these cell lines not only result in PDAC but they reliably metastasize to the liver with a 100% incidence rate within a consistent timeframe. This model allows us to study the entire process of liver metastasis, closely mimicking the human PDAC metastatic cascade. It will not only enable us to investigate the concepts outlined in this proposal but will also serve as invaluable resources for other researchers studying PDAC liver metastasis pathobiology. Unlike the current gold-standard in vivo model of liver metastasis, which uses intrasplenic injection of tumor cells, the approach allows for the study of liver metastasis in a more biologically relevant context, particularly for examining changes in the liver that precede tumor invasion.

Cutting-edge technologies will be used to dissect the molecular mechanisms underlying liver metastasis and tolerance induction. Utilizing the novel oligo-indexed multiplexed imaging technology, formerly known as CODE. The immune-immune and tumor-immune cell interactions at various stages of liver metastatic progression will be investigated. CODEX, an imaging-based method for highly multiplexed cellular phenotyping, will allow us to characterize (i) tissue architecture and the spatial relationships between immune, cancer, and stromal cell types and (ii) the impact of these interactions on the phenotype and activation state of the involved cells. For these studies a new algorithm called CELESTA will be used that combines molecular profiling and spatial location to enable highly accurate CODEX-based determinations of both cellular identities and spatial relationships.

The conceptual notion that EPO-EPOR signaling in macrophages facilitates metastatic progression is groundbreaking. In the early stages of metastasis, EPO-EPOR signaling may drive macrophages toward an immunosuppressive phenotype, supporting the establishment and growth of metastatic tumors. During later stages, this pathway may foster the development of profound immune tolerance, further shielding the tumor from immune-mediated attacks. The approach to developing novel therapeutic strategies that inhibit EPOR signaling aims to reprogram macrophages from an immunoregulatory and tolerance-inducing state to an inflammatory and costimulatory one. This strategy, which is designed to simultaneously elicit an immune attack against the primary tumor and prevent and eliminate liver metastases. is both technically and conceptually innovative.

### Example 2—EPO Determines the Immunosuppressive Immune Landscape in HCC Via EPOR Signaling in Macrophages

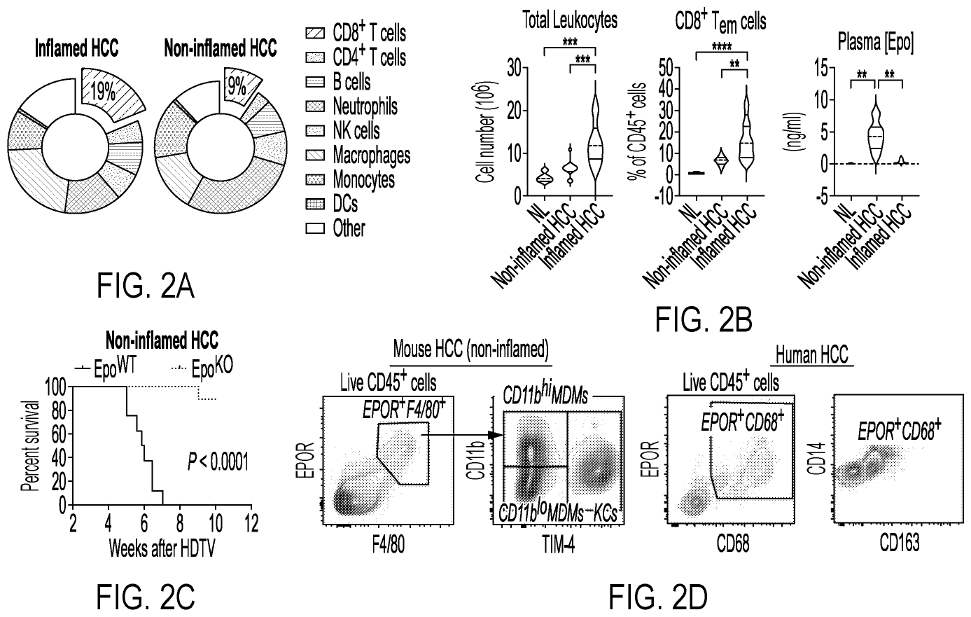

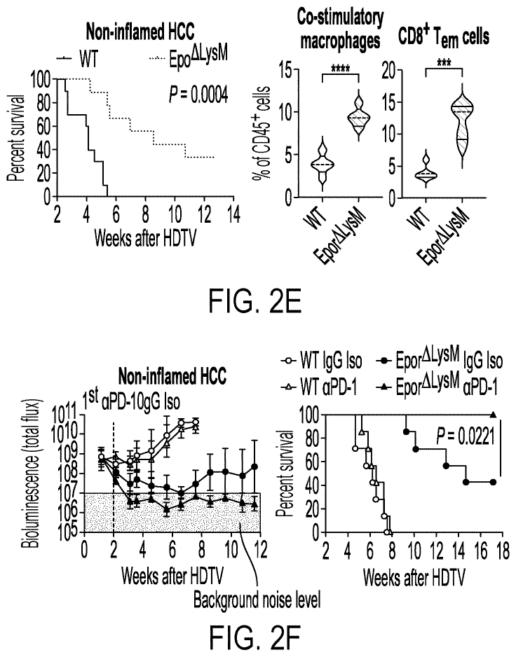

The tumor-derived EPO as a critical barrier was recently identified to effective antitumor immunity in hepatocellular carcinoma (HCC), where EPO functions as an immunosuppressive switch on EPOR+ liver macrophages. HCC accounts for approximately 90% of all primary liver cancers in humans and most HCC patients fail to mount an effective antitumor immune response. To investigate the basis for this failure, spontaneous mouse models of HCC were generated with either an inflamed T-cell-rich or non-inflamed T-cell-deprived tumor Microenvironment™ (FIG. 2A) and compared. The findings from the study using spontaneous mouse models revealed that elevated tumor EPO expression was associated with non-inflamed, T cell-restricted tumors (FIG. 2B). Notably, ablation of EPO in non-inflamed tumors significantly reduced both tumor burden and incidence (FIG. 2C). Given that EPO expression heavily influences the immunotypes of HCC, and that EPOR expression in both human and murine livers is predominantly localized to tissue-resident Kupffer cells (KCs) and recruited monocyte-derived macrophages (MDMs) (FIG. 2D), the role of EPOR signaling in these macrophage populations within primary HCC was further investigated. To decipher the effects of tumor-derived EPO on macrophages, LysM-driven Epor−/− mice (Epor^{ΔLysM}) were generated. The mature myeloid cells, predominantly macrophages in liver and HCC, of LysM-driven Epor−/− mice (Epor^{ΔLysM}) lack EPOR. In this context, the absence of EPOR in macrophages resulted in striking increases in absolute leukocyte infiltration and the percentage of effector memory CD8⁺ T cells and MHCII+CD86+ costimulatory macrophages (FIG. 2E), leading to profoundly improved survival in mice with non-inflamed tumors (FIG. 2E). Restricting EPOR signaling in macrophages effectively converted the TME from non-inflamed to inflamed, rendering the tumors more sensitive to immune checkpoint blockade (ICB) therapy (FIG. 2F). Collectively, these findings revealed that EPO autonomously determines the HCC immunotype and promotes an immunosuppressive TME through activation of EPOR signaling in macrophages. Not only is EPO activation of EPOR on macrophages sufficient for the development of non-inflamed immunosuppressed tumors, but removal of either tumor derived EPO or EPOR from macrophages results in an inflamed tumor. Given the significant role of EPO/EPOR signaling in liver macrophages in shaping the TME of HCC, the EPO/EPOR signaling in macrophages could similarly be leveraged in the context of liver metastases.

### Example 3—EPO-EPOR Signaling in Macrophages Plays a Critical Role in Liver Metastasis

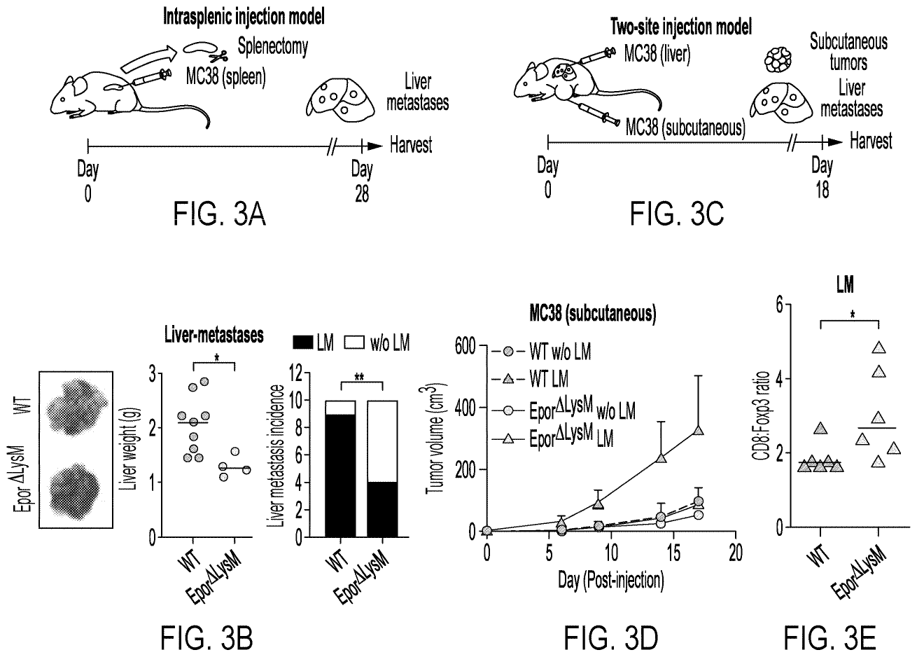

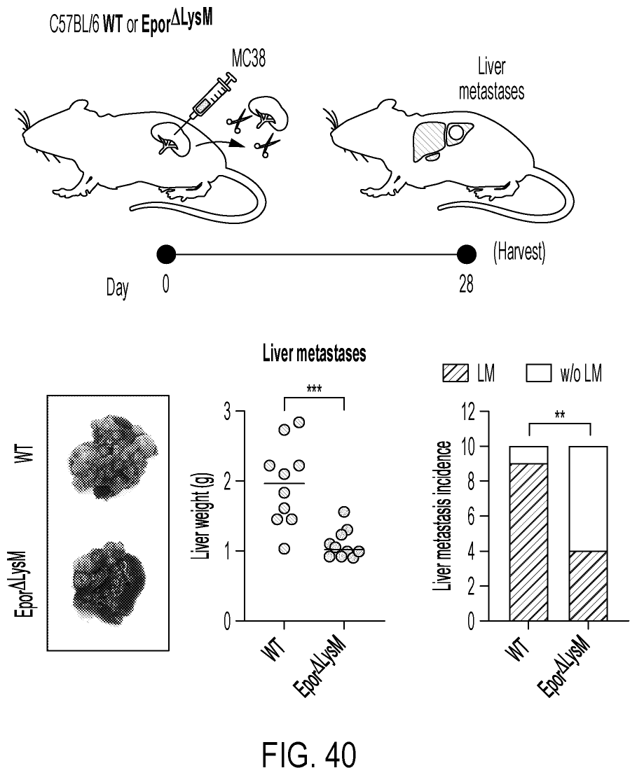

To evaluate the role of EPO-EPOR signaling in macrophages during the early stages of liver metastasis, particularly in metastatic seeding, the conventional intrasplenic injection model was first utilized. This approach involves directly injecting MC38 colorectal cancer cells into the spleen to artificially induce liver metastasis. The livers of wild-type mice were then compared with those of Epor^{ΔLysM} transgenic mice. Consistent with the tumor-protective role of EPOR signaling in macrophages observed in HCC, the absence of EPOR in macrophages significantly reduced both burden and incidence of liver metastasis (FIGS. 3A-3B and FIG. 40). This indicates that EPOR signaling in macrophages may be critical for creating the immunosuppressive liver environment necessary for initial metastatic seeding. In addition to the intrasplenic injection model, other groups have developed a mouse model to investigate how tumors within the liver affect antitumor immunity at distant sites. This model utilizes a two-site tumor system, where tumor cells are simultaneously injected into a subcutaneous site and in the liver of a mouse, mimicking the scenario of advanced tumors with liver metastasis. These studies collectively demonstrated that mice with tumors present within the liver exhibited significantly increased tumor growth of the distant subcutaneous tumors with impaired anti-tumor immunity. This effect is tumor antigen-specific, as injecting different tumor cell lines into the two sites (e.g., MC38 in the liver and B16 melanoma subcutaneously) did not recapitulate the primary tumor growth effects. In the pilot experiments using this two-site injection model, it was similarly observed that established liver metastases accelerated the growth of primary MC38 tumors and lowered the CD8⁺ T cell/Treg ratio. The absence of EPOR in macrophages completely nullified this effect (FIGS. 3C-3D), further suggesting that EPO-EPOR signaling in macrophages is not only crucial for the initial seeding of metastatic tumors but also plays a key role in promoting systemic immune tolerance and tumor growth at distant sites.

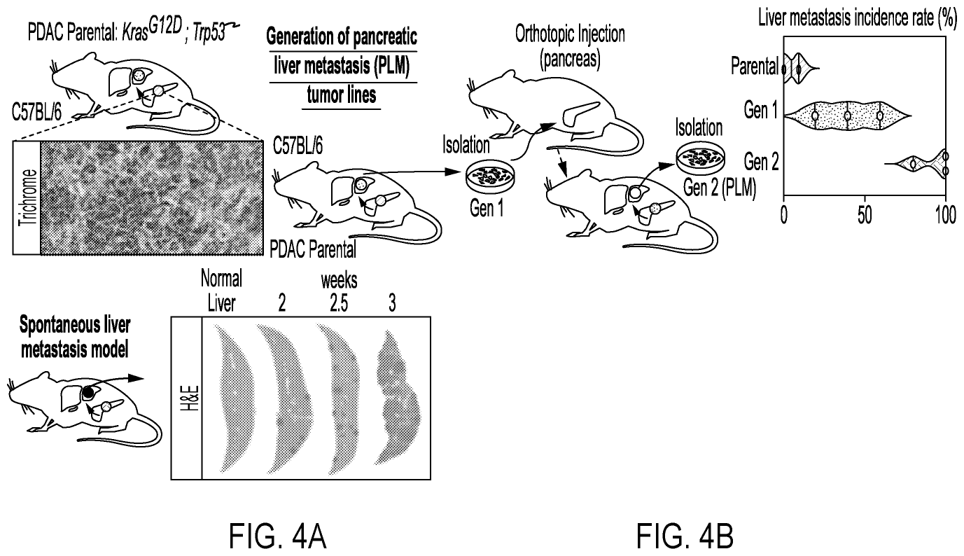

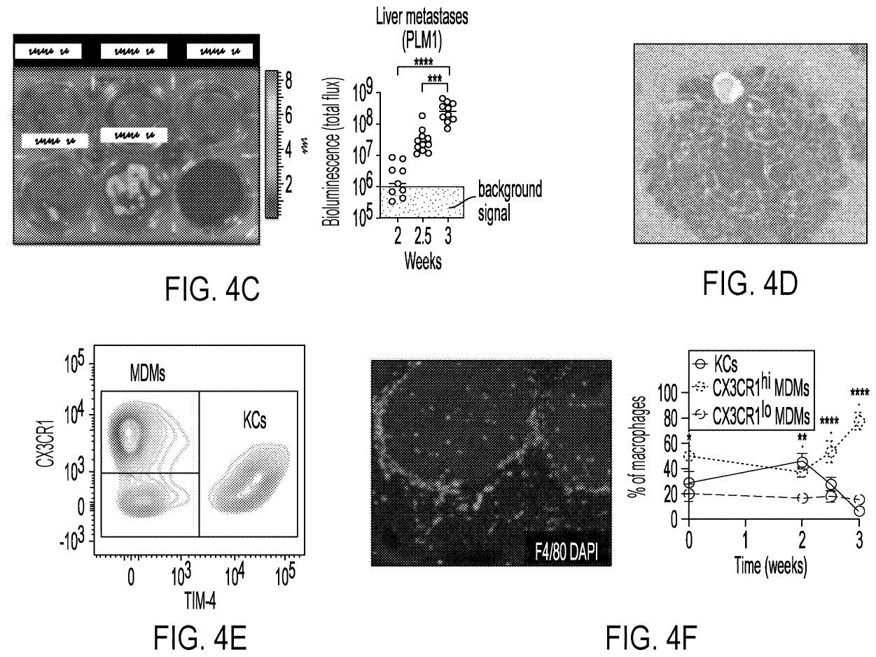

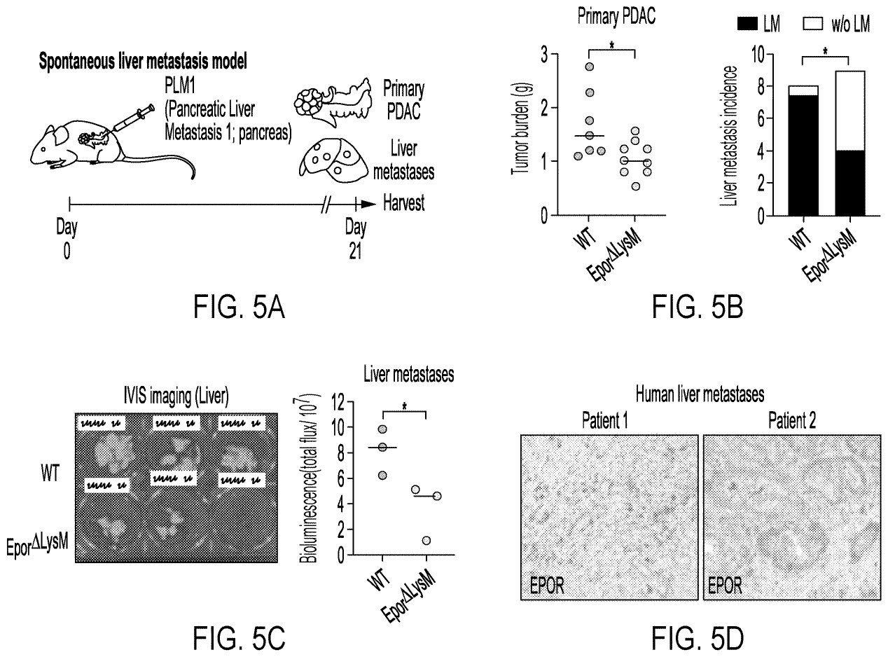

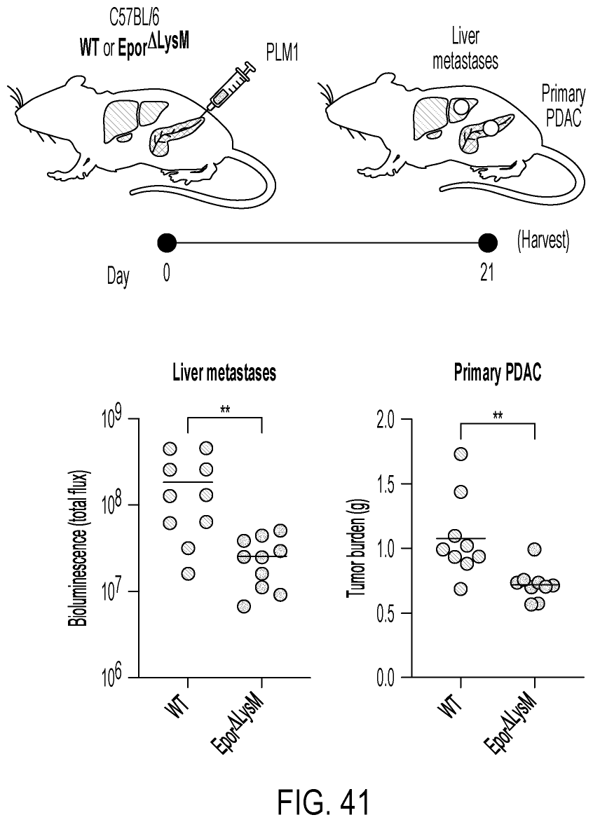

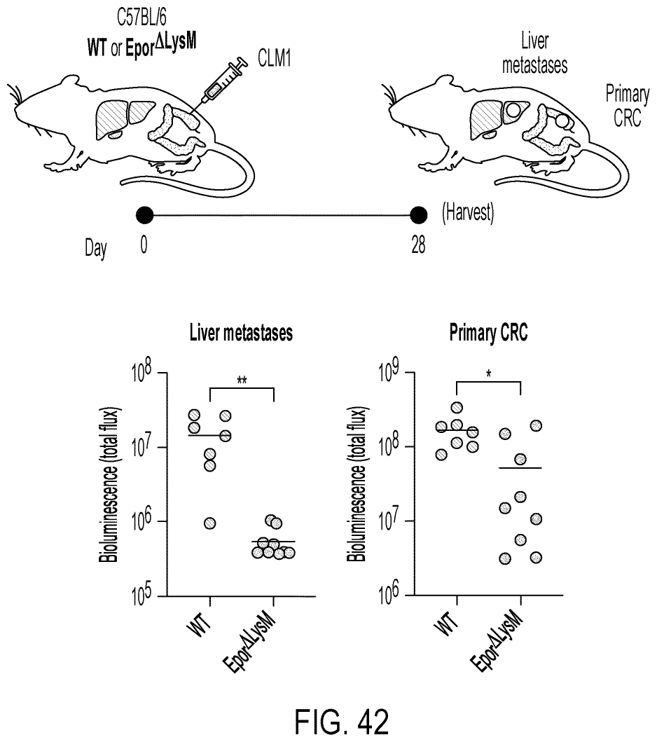

With the goal of generating liver-tropic tumor lines to study liver metastasis, PDAC cell lines were first developed from de novo tumors that formed after electroporating plasmids—containing i) a transposon system for the constitutive expression of KRASG12D and ii) a transient CRISPR-Cas9 system for ablating Trp53 expression—into the pancreas of wild-type mice. When these parental PDAC tumor lines were orthotopically injected into the pancreas of syngeneic hosts, the resulting tumors exhibited a collagen-dense tumor microenvironment (FIG. 4A), reflecting a key pathological feature of human PDAC. A syngeneic melanoma mouse model of lymph node metastasis was previously developed by implanting mice with the minimally metastatic B16-F0 cell line. De novo lines were generated from resultant lymph node metastases and reimplanted into naïve recipients, and this process was repeated over multiple generations to create cell lines with high lymph node metastatic potential. This allowed investigating how tumors spread to lymph nodes and whether lymph node colonization influences metastasis to distant tissues. Using a similar approach, liver-tropic Pancreatic Liver Metastasis (PLM) cell lines (FIG. 4B) and liver-tropic Colorectal Liver Metastasis (CLM) cell lines, were established. When these PLM and CLM cell lines were orthotopically injected into the pancreas (e.g., PLM1) or the colon (or cecum; e.g., CLM1), respectively, they reliably metastasized to the liver within three weeks at a 100% incidence rate (FIG. 4C and FIG. 42). Moreover, similar to histopathology of human liver metastases, PLM nodules formed adjacent to blood vessels (FIG. 4D), further highlighting the strength of this model in recapitulating human disease. Since these PLM and CLM lines were also engineered to express luciferase, the dynamics of liver metastasis can be monitored in real time. Importantly, these PLM and CLM models allow observing temporal changes in the liver microenvironment after primary tumor development in the pancreas or colon but before the formation of visible or detectable metastases in the liver—e.g., PLM1 takes approximately 2 weeks post-orthotopic injection for liver invasion (FIG. 4C)—making it a valuable tool for studying the pre-metastatic niche. Using this new spontaneous liver metastasis model, it was observed not only a shift in the frequency of KCs and MDMs in the liver (FIG. 4E), but also, at the tissue level, an increase in macrophages (F4/80 stain) surrounding and infiltrating liver metastatic nodules (DAPI stain, FIG. 4F). It was confirmed that EPO-EPOR signaling in macrophages is crucial for liver metastasis, as the absence of EPOR selectively in macrophages significantly reduced both the frequency of liver metastatic spread and the growth of liver metastases of PLM1 cells (FIGS. 5A-C and FIG. 41) and CLM1 cells (FIG. 42).

Taken together, the studies using three different liver metastasis models, representing CRC and PDAC, consistently demonstrated that EPO-EPOR signaling in macrophages plays a crucial role in the spread and colonization of primary tumors in the liver, as well as in the immune tolerance induced by liver metastases. Notably, an abundance of EPOR+ populations in human liver metastases was also observed (FIG. 5D). These findings prompt further investigation of: 1) whether EPOR+ macrophages are the primary mediators facilitating liver metastasis, 2) the mechanisms by which EPO-EPOR signaling in macrophages drives tumor-specific immune tolerance, and 3) the potential of pharmacologically targeting EPO-EPOR signaling to treat and prevent liver metastasis.

### Example 4—Evaluating the Role of Macrophages as Gatekeepers in Liver Metastatic Seeding

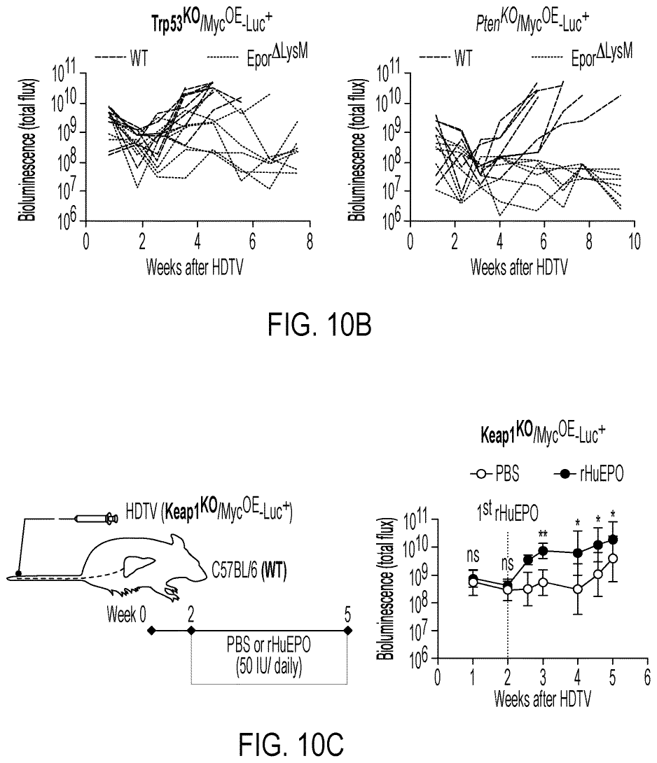

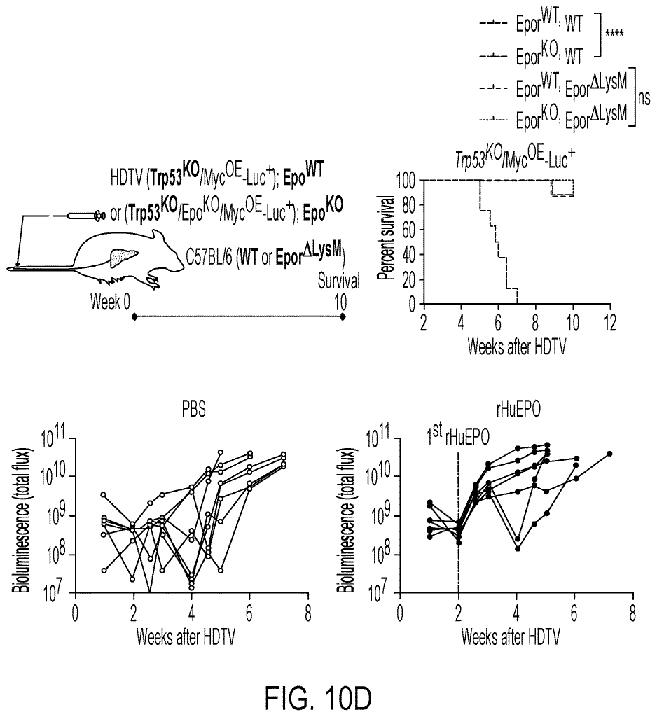

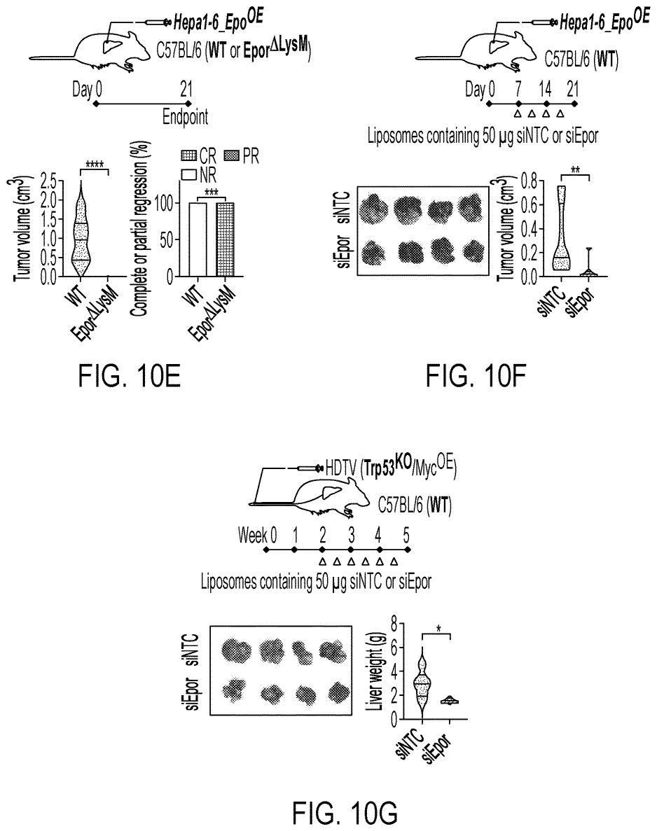

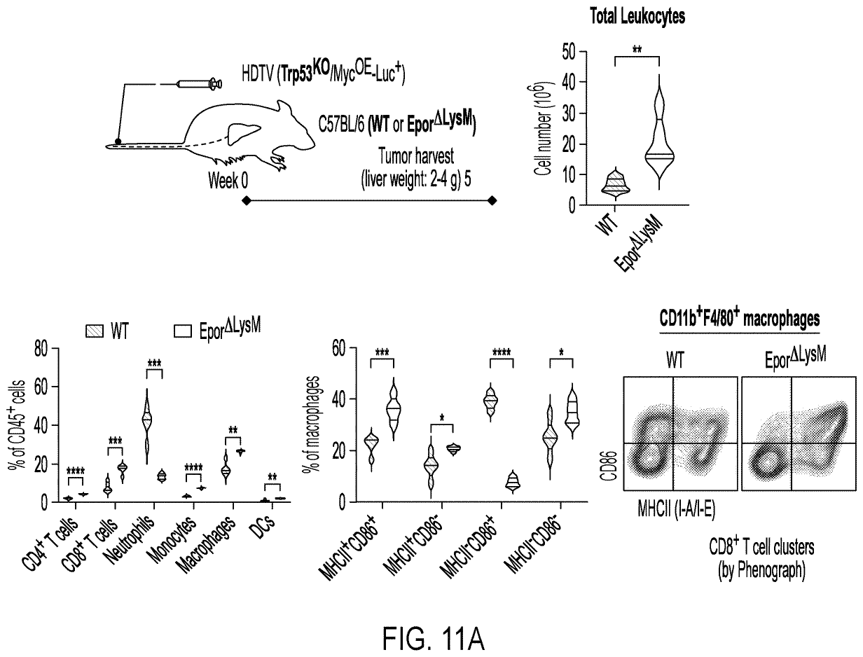

Liver tolerance is largely attributed to its unique microenvironment and the involvement of liver macrophages—KCs and MDMs. To investigate the actions of these cells during the early events of liver metastasis, including seeding, the PLM1 spontaneous liver metastasis model was developed (FIGS. 4A-4C). This model allows speedy and reliable analysis of the temporal changes that occur as tumor cells naturally invade the liver. Experimental setup: To assess whether macrophages play a critical role in early-stage tumor invasion of the liver, 1.5×10⁵ PLM1 cells are orthotopically injected into the pancreas of LysM-iDTR (LysM-cre; inducible diphtheria toxin receptor) mice. Approximately 40% of mice show evidence of liver metastases 2-weeks post-PLM1 injection, with 100% developing sizable metastases by 2.5-weeks (FIG. 4C). Macrophage depletion are achieved via intraperitoneal (i.p.) injections of diphtheria toxin (DT) at 25 μg/kg every 48 hours. To evaluate the effects of macrophage depletion during different stages of early metastatic disease, ranging from pre-metastatic livers to those with small tumor nodules, DT treatment can commence on day 3, 7, or 10 post-PLM1 injection. Initial studies are performed on primary tumors, livers, and spleens to confirm macrophage depletion efficiency and specificity. After confirmation of macrophage depletion with this method, in separate studies mice are euthanized 3-weeks post-PLM1 injection, and their livers are harvested for analysis, including assessment of metastatic frequency and size by bioluminescence IVIS imaging and H&E histology, as well as downstream applications such as CODEX/immunohistology, flow cytometry, and single-cell RNA sequencing for other aims of this proposal. Additionally, to determine if the observed effects are specifically driven by tissue-resident macrophages, parallel experiments are conducted using Clec4f-iDTR mice, which allow for the conditional depletion of KCs. Rigor and reproducibility: Based on the preliminary findings from the intrasplenic injection studies (FIGS. 2A-2B), 10 mice per group are required to establish statistical significance with an alpha of 0.05 and power of 0.9. Predicted results: Since the preliminary studies showed that disrupting macrophage function markedly reduced both the incidence and burden of liver metastasis, it was reasoned that macrophages are the primary tolerogenic mediators of metastasis in the liver. Therefore, depleting macrophages can lead to significantly lower the rate of metastatic seeding and growth. The systemic administration of DT may induce toxicity beyond the intended depletion of macrophages and could potentially affect cancer cell growth. To ensure that observed effects are not artifacts of DT treatment, macrophages will be depleted using clodronate liposomes, which have been proven effective in specifically targeting liver macrophages. Macrophages can be critical for pre-metastatic niche formation and metastasis maintenance, and it remains unclear whether macrophage recruitment to the liver is a key component of this process. To address this uncertainty, CCR2^{ΔLysM} or CX3CR1ΔLysM mice (in-house available) will be used to disrupt macrophage recruitment into the liver. In doing so, whether tissue-resident or recruited macrophages influence pre-metastatic niche formation to aid tumor seeding in the liver will be assessed.

### Example 5—Investigating EPO Induced EPOR Activation in Macrophages as a Key Driver of Pre-Metastatic Niche Development in Liver

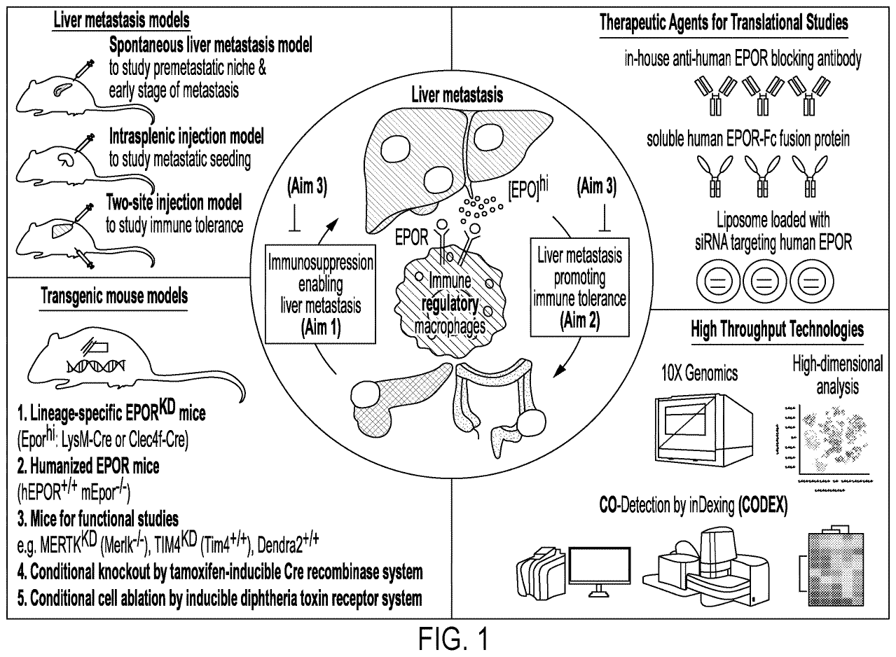

Macrophages play a crucial role not only within primary tumors but also at secondary pre-metastatic sites. Although tumor-derived extracellular vesicles and soluble factors have been reported to remotely polarize macrophages toward pre-metastatic niche development, the key signaling pathways involved remain largely unknown. While tumor-derived EPO locally converts macrophages into an immunoregulatory phenotype in HCC (FIG. 1D), it is unclear whether primary tumors in distal tissues, such as the pancreas, employ the same signaling mechanism to remotely prime liver macrophages for metastatic invasion. To assess whether tumor-derived EPO is associated with the incidence of liver metastases, both loss-of-function (EPO knockout) and gain-of-function (EPO activation) experiments will be conducted. Specifically, CRISPR interference (CRISPRi) will be used to genetically ablate EPO (EPO^{KO}) and CRISPR activation (CRISPRa) to induce EPO expression (EPOOE) in the PLM1 cell line. To determine if macrophage-expressed EPOR is the primary downstream mediator of EPO in liver metastasis, Epor^{ΔLysM} mice, in which all macrophages lack EPOR, will be utilized. A total of 1.5×10⁵ PLM1-parental, -EPO^{KO}, or -EPO^{OE} cells will be orthotopically injected into the pancreas of wild-type or Epor^{ΔLysM} C57BL/6 mice (in-house available). Mice will be euthanized 3-weeks post-PLM1 injection, and their livers will be examined for metastatic frequency and burden as outlined. Harvested livers will be subjected to (i) immune cell profiling by flow cytometry, (ii) analysis of changes in spatial distribution of immune cells and stromal cells by immunohistology/CODEX and (iii) evaluation of transcriptional alterations in various immune cell populations through single-cell RNA sequencing (scRNA-seq) analysis. Peripheral blood will also be collected to analyze EPO concentrations via ELISA. For flow cytometry analysis, single-cell suspensions from the livers and peripheral blood will be profiled to determine the frequency of different lymphocyte and myeloid cell subsets, with a specific focus on macrophage functional status (e.g., CD206, CD163, CD86, CD80, MHCII, IL-10, IL-1A, TGF-β, MERTK, FASL). To evaluate the importance of KCs, Epor^{ΔClec4f} mice will be used in parallel experiments instead of Epor^{ΔLysM} mice. Rigor and reproducibility: Based on the preliminary findings from the intrasplenic injection studies (FIGS. 2A-2B), 10 mice per group are required to establish statistical significance with an alpha of 0.05 and power of 0.9. Expected results: Given that tumor-derived EPO and macrophage-expressed EPOR are crucial for generating an immunosuppressive TME in HCC, the removal of either molecule will lead to a decrease in the incidence and burden of liver metastasis. Furthermore, it is expected that in the absence of EPO-EPOR signaling, macrophages will shift toward an immunostimulatory phenotype. While genetically manipulating EPO expression in cell lines allows investigating the overall influence of tumor-derived EPO on liver metastases, it remains unclear whether tumor-derived EPO: (i) acts remotely on the pre-metastatic site to enhance tumor seeding, or (ii) simply promotes unrestrained nodule growth post-metastases. To address this, wild-type mice implanted in the pancreas with PLM1-EPOKO cells will receive daily administrations of 50 IU of recombinant murine EPO (rmEPO). To delineate the timing of EPO's action, rmEPO treatment will begin on day 3, 7, or 10 following PLM1 injection; and PBS will be used as the control treatment. Mice will be euthanized a week following treatment initiation. It will be assessed whether EPO treatment expedites tumor seeding in the liver and/or enables exacerbated nodule growth or both. Complementary approach 2: While these experiments interrogate the function of EPO, whether EPO-EPOR signaling in macrophages is crucial for metastatic seeding or the growth of established metastatic nodules remains unclear. To control the timing of EPOR ablation in macrophages, Epor^{ΔLysM}-ER[T2] mice (in-house available), in which EPOR deletion in macrophages is tamoxifen-inducible, will be used. Tamoxifen will be administered (75 mg/kg) starting at 0-, 1-, or 2-weeks post-PLM1 injection, with corn oil serving as the vehicle control. Mice will be euthanized 3-weeks post-PLM1 injection. This experimental setup allows assessing the crucial timing in which macrophage EPOR signaling assists in metastatic seeding and nodule growth.

### Example 6—Delineating the Temporal Changes in Macrophage Phenotype and Cell-Cell Interactions in Pre-Metastatic Niche and Early Liver Metastases

The pre-metastatic niche comprises a variety of cell types that collectively contribute to forming a microenvironment favorable to tumor seeding at a distal secondary site. While some studies have underscored the significance of external signaling on macrophages during this process, comprehensive investigation of the temporal changes at pre-metastatic sites remain underexplored due mainly to the lack of an appropriate model. To investigate whether macrophages i) localize (or enrich) near blood vessels, ii) change expression of functional markers, and iii) display altered cell-cell interaction(s) prior to tumor liver invasion, liver samples from mice bearing PLM1 tumors, described in Examples 3-5, will undergo (i) scRNA-seq analysis and (ii) CODEX immunohistology analysis. For the scRNA-seq experiments, half of the harvested livers will be dissociated and enriched for CD45+ immune cells using magnetic-activated cell sorting, while the other half will be fixed and processed into tissue sections for CODEX analysis. CODEX utilizes DNA barcoding in conjunction with immunostaining and fluorescence microscopy, allowing for the imaging of over 60 targets within a single tissue section. The multiplexed nature of CODEX imaging allows for the evaluation of all major leukocyte and stromal subtypes, with a focus on immune cells, including T cells (CD3), T cell subsets (CD4 and CD8), regulatory T cells (Treg; CD25 and FOXP3), B cells (CD19 and B220), macrophages (F4/80), Kupffer cells (CLEC4F), classical type-1 dendritic cells (cDC1; CD11c and XCR1), cDC2 (CD11c and CD11b), NK cells (NK1.1), plasmacytoid DCs (SIGLEC-H and B220), monocytes (LY-6C), neutrophils (LY-6G), eosinophils (SIGLEC-F and IL-5R), and phenotypic markers of macrophage polarization (e.g., CD206, CD163, ARG1, HMOX1 and TIM-4), exhaustion (e.g., PD-1, PD-L1, TIM-3, TIGIT and ILT3), co-stimulatory molecules (e.g., CD40, CD80, CD86, MHCI, MHCII), cytokines (e.g., IFN-γ, TGF-β, IL-4 and IL-10), and chemokine receptors that may alter cell trafficking (e.g., CCR2, CCR7 and CXC3CR1). EPOR, will be also included, and a monoclonal antibody specific for human EPOR that cross-reacts with mouse EPOR will be utilized. By overlaying transcriptomic data with histological data, the temporal phenotypic changes occurring in cell populations within a biologically and spatially relevant context will be studied. Putative unique cell markers identified during scRNA-seq experiments will be incorporated into CODEX analyses as appropriate antibodies become available. Rigor and reproducibility: In human head and neck squamous cell carcinoma (HNSCC) tissues (n=4 per group), a unique cell-cell interactions were identified previously when comparing two sample groups using CELESTA algorithm. Building on these findings, four samples per group for CODEX experiments will be collected. Antibodies for CODEX and subsequent immunohistology experiments will undergo rigorous validation prior to use. Imaging data will be made publicly available on the Synapse platform (AMP-AD Knowledge Portal). Expected results: Building on the preliminary studies that demonstrate (i) EPO-stimulated macrophages acquiring an immunoregulatory phenotype (FIG. 2D) and (ii) macrophages surrounding liver-invading tumor nodules (FIG. 4E), it is expected that CODEX experiments will reveal a macrophage-dominant niche near blood vessels. Furthermore, these macrophages are likely to adopt and exhibit an immune-regulatory phenotype prior to tumor nodule formation. Complementary approach 1: While macrophages are hypothesized to play a significant role in the early stages of liver metastases, it is plausible that other non-immune cells in the liver—such as endothelial cells (CD31) and stromal cells, including mesenchymal stem/stromal cell-like (CD44, CD73, CD90, CD105), myofibroblast-like (αSMA), pericyte-like (PDGFR), and matrix-remodeling (FAP) cells—also contribute to pre-metastatic niche formation. Disruption of EPOR signaling in macrophages is expected to alter cell-cell interactions and the composition of the cellular niche. In this context, spatial transcriptomics using Xenium by 10× Genomics will be performed on appropriate serial sections to identify changes in tissue architecture.

### Example 7—Evaluating EPO-EPOR Signaling at the Metastatic Site as a Driver for Immune Tolerance Induction

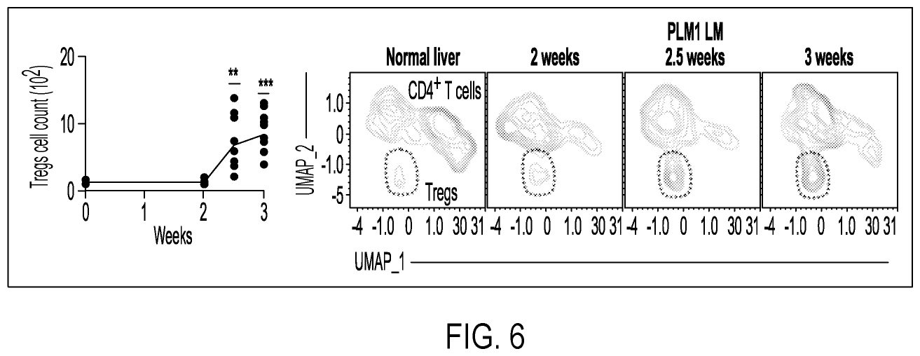

Clinically, liver metastasis is a significant adverse event associated with reduced patient survival. Although recent studies have begun to explore the pathology of liver metastases, the mechanisms driving disease progression remain elusive. The preliminary findings using the two-site injection model also revealed that liver metastasis induces the development of Tregs that confer systemic tumor-specific immune tolerance in the form of primary tumor outgrowth (FIGS. 3C-3D). Notably, primary tumor growth is exacerbated only when the same tumor was also present in the liver (FIGS. 3C-3D). Additionally, primary tumor outgrowth was observed only in wild-type mice, characterized by a decreased CD8+/Treg ratio, but not in Epor^{ΔLysM} mice (FIG. 3E). Last, the preliminary studies utilizing the spontaneous liver metastasis model, PLM1, revealed that Tregs enrich in livers as a consequence of tumor invasion (FIG. 6). Taken together, the metastasis-derived EPO signals through macrophages to promote the development of Tregs in the liver, which then disseminate to promote systemic tumor-specific immune tolerance.

The liver is a unique organ capable of inducing both local and systemic immunological tolerance towards foreign antigens. This tolerogenic effect is attributed to specialized immune cells within the liver, particularly KCs and MDMs, which express immunoregulatory molecules that promote Treg development. Although the preliminary work in HCC revealed that liver macrophages acquire an immunomodulatory phenotype through EPO-EPOR signaling (FIG. 2D), it remains unclear whether EPO-EPOR signaling in the liver mediates the induction of tumor-specific immune tolerance. To address this question, the two-site injection model will be employed. Specifically, to determine whether EPO production at metastatic nodules or the primary tumor promotes tumor-specific immune tolerance, the genetic engineering strategies will be used to knockout (KO) or overexpress (OE) EPO in the MC38 cell line. C57BL/6 wild-type mice will then be injected with either 5×10⁵ MC38-EPO^{KO} cells subcutaneously, 5×10⁵ MC38-EPO^{OE} cells into the liver, or the reverse. As a control, PBS will be injected into the liver instead of cells to mimic conditions without liver metastasis. Tumor growth will be monitored every other day using calipers. Two-weeks post-implantation, primary tumors and livers will be harvested and analyzed via flow cytometry for general immune cell profiling, with a specific focus on the activation status of Tregs (e.g., CD3, CD4, CD25, FOXP3, HELIOS, CTLA-4, CCR8, ICOS, GARP, GITR). Tregs are essential for maintaining and mediating immune tolerance toward both self and foreign antigens. To confirm the role of Tregs in primary tumor outgrowth, as suggested by the results in FIG. 3E, Tregs will be depleted in the two-site injection model. Mice will receive 2 mg/kg of either αCD25 (clone: PC-61.5.3; for targeting general Tregs), αCTLA-4, or αCCR8 (clone: 9H10, SA214G2; for targeting tumor-specific Tregs) antibody, starting at 3-, 6-, 9-, and 12-day post-implantation. Primary tumors, livers, and spleens will then be harvested to confirm Treg depletion efficiency and specificity. Finally, the two-site injection model will be implemented in Epor^{ΔLysM} and Epor^{ΔClec4f} mice to confirm the involvement of macrophages, particularly KCs, in the process of liver-mediated systemic tumor-specific immune tolerance. Rigor and reproducibility: Based on preliminary studies using the two-injection site liver metastasis model (FIGS. 3C-3E), 10 mice per group are required to establish statistical significance with an alpha of 0.05 and power of 0.9. Expected results: In the tumor-derived EPO manipulation experiments, it is expected that exacerbated primary tumor growth will depend on two key factors: (i) the expression of tumor-derived EPO at the secondary (metastatic) site, and (ii) the presence of the same tumor in the liver. It is expected that Treg depletion will attenuate this effect, even if the tumor in the liver exhibits elevated EPO expression. Similarly, disrupting macrophage EPOR signaling is expected to restrict primary tumor growth. Although these experiments focus on the role of EPO-EPOR signaling in metastasis-induced immune tolerance, it is plausible that lymph nodes, which serve as educational hubs for the immune system, may also contribute to the development of tumor-specific Tregs. To rule out the involvement of lymph nodes in this process, parallel experiments will be performed using LTa−/− mice (commercially available), which lack lymph nodes. In addition, B16F10-EPO^{KO} and B16F10-EPO^{OE} cell lines will be also generated to perform the same procedures with B16F10-derived cells to validate the results from the two-site injection model using MC38 derived cell lines.

### Example 8—Assessing Macrophages as Mediators of Metastasis-Induced Tumor-Specific Treg Development

Liver macrophages, particularly tissue-resident KCs, play a crucial role in mediating immune tolerance through mechanisms such as (i) efferocytosis and (ii) antigen presentation. While the role of liver macrophages, especially in the context of liver transplantation, has been studied, it remains unclear whether these tolerogenic mechanisms are exploited during liver metastasis. Efferocytosis, the phagocytosis of apoptotic cells, is hypothesized to induce immune tolerance. To determine if macrophages are the predominant cells that engulf metastatic tumor cells in the liver, MC38 cells transduced to express the ZsGreen fluorescent protein will be used. A total of 5×10⁵ MC38-ZsGreen cells will be administered via intrasplenic injection. Two-weeks post-injection, mice will be euthanized, and their livers will be analyzed by flow cytometry to identify which phagocyte populations have taken up the tumor cells. Phosphatidylserine expressed on apoptotic cells is recognized by receptors on macrophages, such as TIM-4, and via the MERTK endocytosis pathway, allowing apoptotic cells to be engulfed and transported into endosomes for processing. To determine whether disrupting efferocytosis impacts tolerance induction, Timd4^{+/-} and MerTK^{-/-} mice will be utilized in parallel experiments. Next, to determine whether macrophages mediate antigen presentation for tumor-specific Treg development, MC38 cells transduced to express chicken ovalbumin (OVA) will be used to perform similar experiments as previously described. Additionally, I-AB^{ΔLysM} and I-AB^{ΔClec4f} mice, which lack MHCII expression in macrophages, will be included to further investigate the role of MHCII in antigen presentation by macrophages. I-Ab OVA 323-339 tetramer antibodies will be used to assess the frequency of tumor-specific Tregs by flow cytometry. Lastly, in parallel experiments, the two-site injection model will be implemented to determine whether restricting MHCII expression in macrophages limits the metastasis-induced outgrowth of the primary tumor. Subcutaneous tumors will be measured twice a week using calipers. Two weeks post-liver injection, the livers and primary tumors will be harvested and analyzed by flow cytometry. Based on preliminary studies using the two-injection site model (FIGS. 3C-3E), 10 mice per group are required to establish statistical significance with an alpha of 0.05 and power of 0.9. Given that macrophages are the predominant phagocytic cells in the liver, both KCs and MDMs are expected to exhibit the highest levels of green fluorescence among immune cell populations. By disrupting components of efferocytosis, a reduction in both the percentage and intensity of green fluorescence is expected. This disruption is also expected to attenuate tolerance induction, as evidenced by a decrease in primary tumor growth and a reduction in the frequency or count of Tregs. Consistent with the hypothesis that macrophages are critical for the development of tumor-specific Tregs in the liver, abrogating macrophage MHCII expression is likely to hinder the formation of these Tregs. While these experiments aim to dissect the role of macrophages, the potential enhancement of macrophage tolerogenic functions—such as efferocytosis and Treg induction—via EPO signaling remains to be investigated. To determine whether EPO-EPOR signaling influences macrophage phagocytic capacity, MC38-ZsGreen cells will be injected into the spleens of wild-type or Epor^{ΔLysM} mice and whether restricting EPOR signaling in macrophages decreases the percentage of green-fluorescent cells will be assessed. Additionally, KCs and MDMs harvested from mice with MC38-OVA liver metastases will be co-cultured with CD4⁺ T cells from FoxP3-GFP OT-II mice. Treg expansion will be evaluated using CellTrace™ Violet and analyzed by flow cytometry. To determine whether EPO exerts similar effects on human macrophages, human Kupffer cells or PBMC-derived macrophages will be pre-treated with rhEPO in vitro. These cells will be assessed by flow cytometry for i) co-stimulatory and immunoregulatory markers, ii) phagocytic capacity and antigen presentation of irradiated pHrodo-SE-labeled tumor cells, and iii) their ability to activate and expand Tregs. Additionally, scRNAseq data from macrophages in human and mouse liver metastasis samples will be phenotypically compared.

### Example 9—Investigating Liver Metastases as an Educational Hub for Treg Development

Secondary lymphoid organs function as educational hubs for antigen-specific adaptive immunity. However, whether antigen-presenting cells, (e.g., macrophages and dendritic cells) migrate out of the liver to educate Tregs, or rather the liver itself can function as an educational hub and Tregs migrate out of the liver remains elusive. First, to determine whether macrophages or Tregs migrate out of the liver, Dendra2 mice will be injected with 5×10⁵ MC38 cells into both the liver and subcutaneous tissue (two-site injection model). Dendra2 is a photoactivatable protein that shifts from green to red fluorescence upon exposure to ultraviolet light. Using fiber-optic-based light sources, leukocytes will be selectively photoconverted within the liver and liver metastases at specific time points, which will allow tracking which leukocytes migrate from the liver to organs of interest (e.g., secondary lymphoid organs and distant tumors). On 0-, 3-, 7-, and 10-day post-implantation, leukocytes will be photoconverted in the liver and liver metastases, and mice will be euthanized 24-hours post-photoconversion. The livers, portal and celiac lymph nodes, spleens, and primary tumors will be harvested, and leukocyte trafficking will be examined with flow cytometry analysis. Second, to determine whether tertiary lymphoid organ-like structures can be developed in the liver metastases, liver metastases will be analyzed from various models, such as the PLM1 spontaneous liver metastasis model or the intrasplenic injection model of MC38, using CODEX. Liver sections will be examined for CD4+ T cell/Treg clusters or areas with increased interaction between CD4+ T cells and macrophages (F4/80) or Kupffer cells (CLEC4F). Serial sections from tissues displaying these cellular niches or architectures will be further analyzed using CODEX assays. Rigor and reproducibility: Based on preliminary studies using the two-injection site liver metastasis model (FIGS. 3C-E), 10 mice per group are required to establish statistical significance with an alpha of 0.05 and power of 0.9. Expected results: Given that secondary lymphoid organs are key education hubs for antigen-specific adaptive immunity, it is plausible that antigen-presenting cells, such as macrophages or dendritic cells, migrate out of the liver and travel to secondary lymphoid organs. The experiments using LTa−/− mice and Dendra2 mice are expected to complement each other in addressing whether the migration of antigen-presenting cells to secondary lymphoid organs (e.g., lymph nodes) is necessary for the development of tumor-specific Tregs in liver metastasis. However, an alternative perspective suggests that the liver itself functions as a lymphoid organ, meaning macrophages may not need to leave the liver to educate CD4⁺ T cells. Based on this, it is hypothesized that interactions between macrophages and CD4+ T cells, including Foxp3+CD4⁺ T cells (i.e., Tregs), are likely enriched near the invasive edges of metastatic nodules, as the preliminary studies have shown that macrophages densely surround these areas (FIG. 4E). Immunophenotyping by flow cytometry (or immunohistology/CODEX) can distinguish general features of immune cells, such as inflammatory versus immunomodulatory macrophages. To better understand the differences and phenotypic and transcriptional changes between normal, pre-/early-metastasis, and late-metastasis liver macrophages, scRNA-seq will be used to unbiasedly identify differentially expressed genes among macrophages and other cell populations within and between samples (control, pre-/early-metastasis, and late-metastasis). By identifying potential unique cell population markers for CODEX experiments, functional changes can be annotated in a spatially relevant manner and the temporal significance of cell-cell interactions and communication can be understood.

### Example 10—Examination of Pharmacological Inhibition of EPO-EPOR Signaling as an Effective Strategy to Prevent and Treat Liver Metastasis

Liver metastases present a major clinical challenge, since current preventive and treatment regimen are of limited benefit. Although immune checkpoint blockade (ICB) therapies can be effective for some metastatic diseases, patients with liver metastases typically exhibit reduced responsiveness to these treatments. The preliminary data with liver metastasis models demonstrate increased infiltration of immunoregulatory macrophages in liver metastases, with elevated levels detectable as early as the pre-metastatic niche (FIGS. 4D-4E). Also, the previous study demonstrated that genetic ablation or pharmacological inhibition of EPOR in macrophages sensitize ICB-resistant HCC to ICB therapy (FIG. 2E), and abrogating EPOR in macrophages reduced liver metastasis rate and burden (FIGS. 3 & 5). Based on these findings, the pharmacologic targeting of the EPO/EPOR pathway could serve as an effective preventive and therapeutic strategy for liver metastases, as well as enhancing responsiveness of liver metastases to ICB.

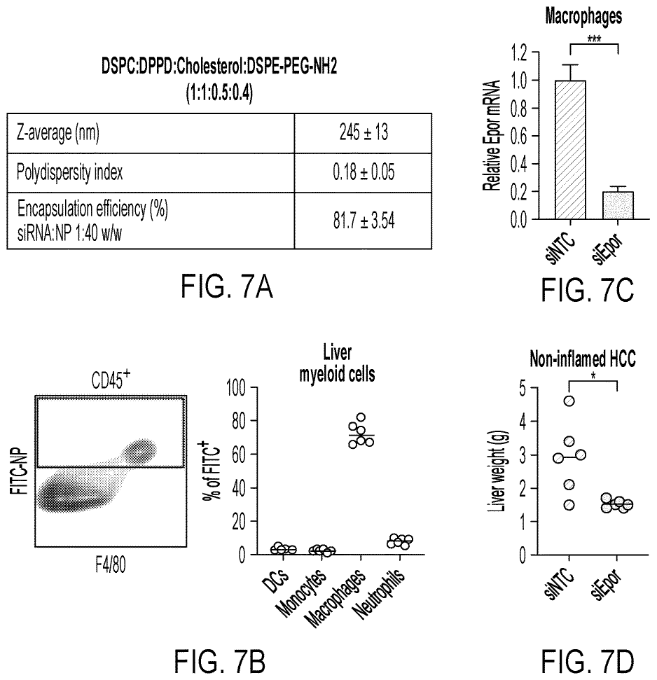

The EPOR⁺ macrophages play a significant role in HCC, and that pharmacological inhibition of the EPO/EPOR pathway leads to dramatic tumor reduction. Whether these effects extend to liver metastasis remains to be determined. To enhance the translational relevance of the studies, humanized EPOR mice (hEPOR; obtained from C. T. Noguchi at NIH and bred in the facility), in which the mouse EPOR is replaced by human EPOR, will be used to evaluate the efficacy of EPO/EPOR-targeting agents. Three therapeutic approaches will be tested in the hEPOR mice: (i) soluble human EPOR-Fc fusion proteins (sEPOR-Fc), which act as decoy receptors to neutralize EPO; (ii) neutralizing anti-EPOR monoclonal antibodies (αEPOR); and (iii) siEPOR-loaded lipid nanoparticles (siEPOR-NPs), which, when larger than 200 nm in diameter, are preferentially taken up by phagocytes, particularly macrophages, enabling macrophage-specific reduction of EPOR (feasibility; FIG. 7). First, to test if the therapeutic agents prevent liver metastasis, hEPOR mice will receive an intrasplenic injection of 5×10⁵ MC38 cells or an orthotopic injection of 1.5×10⁵ PLM1 cells, followed by treatment with either sEPOR-Fc (20 mg/kg i.p. twice per week) or αEPOR antibody (2 mg/kg i.p. twice per week) from 3-day post-injection until the end of the experiment. Livers will be harvested at 3-weeks to evaluate the incidence of liver metastasis. Next, for treating established liver metastases, agents will be administered starting 2-weeks post-injection of MC38 or PLM1 cells until the end of the experiment, and livers will be harvested at 4-weeks for quantification of metastasis burden using bioluminescence IVIS imaging. Changes in the immune landscape of the treated liver tissues will be assessed using flow cytometry and immunohistology. Blockade of the EPO/EPOR pathway may lead to anemia since EPO is crucial for red blood cell production by stimulating EPOR on erythroid progenitor cells in the bone marrow. However, this risk should be manageable due to the long half-life of mature red blood cells, which do not express EPOR and typically circulate for about 60 days in mice and 120 days in humans, and the high affinity of EPO for its receptor on erythrocyte progenitors compared to its receptor on other cell types including macrophages. Thus, anemia is unlikely to develop rapidly if at all. Nonetheless, to assess the risk of anemia, mice injected with the above agents will be monitored with weekly small volume blood collections for complete blood count analysis. Based on preliminary studies on relevant models, 10 mice per group are required to establish statistical significance with an alpha of 0.05 and power of 0.9. Given that EPOR signaling is critical for liver immunosuppression and that EPOR knockout in macrophages reduces liver metastasis rate and burden in the mouse models, it is expected that both EPO/EPOR-targeting agents will exhibit similar prophylactic and therapeutic effects. It is also expected that these therapeutic agents can reprogram liver macrophages from an immunoregulatory to a costimulatory state, thereby enhancing the infiltration of tumor-killing effector cells.

Patients with liver involvement consistently exhibit poorer responses to treatments, including ICB. Supporting this observation, liver metastasis in mice has been shown to promote primary tumor growth outside the liver and impair ICB effectiveness by macrophage-dependent CD8⁺ T cell dysfunction and recruitment of tumor-specific Tregs. In HCC, it was demonstrated that EPO/EPOR signaling prompts macrophages to adopt an immunoregulatory phenotype, maintaining a non-inflamed tumor microenvironment (TME) that is unfavorable to ICB. Notably, the ablation of the EPOR in macrophages is sufficient to induce an inflamed TME and enhance antitumor T-cell immunity, thereby synergizing with ICB. EPO/EPOR signaling also represents a major obstacle to ICB in the context of liver metastasis. Two liver metastasis models will be employed: the MC38 two-site injection model and the orthotopic injection of PLM1 cells. Starting 1-week post-injection, Epor^{ΔLysM}-ER[T2] mice will receive tamoxifen to induce EPOR knockout specifically in macrophages. Concurrently, the mice will be treated with 2 mg/kg i.p. of αPD-1 antibody twice weekly. After three weeks, primary tumors and liver tissues will be harvested for immune cell profiling using high-dimensional spectral flow cytometry. The analysis will include general immune cell types, with an extended focus on T cell activation and memory markers (e.g., TCF1, EOMES, TNF-α, IFN-γ, KLRG1, CD69, CD44, CD62L, Ki67, PD-1, TIGIT, TIM-3, CTLA-4) and macrophage functional markers. To determine T cell antigen specificity, MC38-OVA and PLM1-OVA cells will also be utilized. Additionally, a separate survival experiment will be conducted to assess whether the ablation of EPO/EPOR in macrophages sensitizes tumor-bearing mice with liver metastasis to ICB, potentially prolonging survival and achieving a pathological complete response. Tumor growth dynamics will be monitored using bioluminescence IVIS imaging. Rigor and reproducibility: Based on preliminary studies on relevant models, 10 mice per group are required to establish statistical significance with an alpha of 0.05 and power of 0.9. Since EPOR+ macrophages significantly contribute to immune tolerance and resistance to immune checkpoint blockade (ICB) in the liver, it is expected that blocking EPOR signaling in macrophages will sensitize both liver metastases and primary tumors to αPD-1 therapy, potentially leading to improved overall survival and even disease-free survival. Additionally, it is expected that tumors will exhibit a higher frequency of tumor-specific effector T cells with enhanced differentiation and activation. By administering tamoxifen, the Epor^{ΔLysM}-ER[T2] mice model allows for the selective knockout of EPOR in macrophages after tumor establishment. This approach enables the investigation of the therapeutic potential of targeting EPOR in macrophages within the tumor microenvironment, while preserving macrophage function during early development. However, it is important to consider that tamoxifen may have off-target effects, toxicity, or could potentially interfere with αPD-1 antibody therapy. A combination therapy using αPD-1 antibody and EPO/EPOR-targeting agents will be tested in wild-type C57BL/6 mice across the same liver metastasis models.

### Example 11—Non-Inflamed Tumors Preferentially Secrete EPO

Spontaneous tumor models, each with a defined inflamed or non-inflamed tumor microenvironment (TME), were developed utilizing genome-editing approaches. The gene-editing approaches combined the delivery of transposase-transposon vectors for Myc overexpression (Myc^{OE}) and CRISPR-Cas9 systems by hydrodynamic tail vein injection (HDTV) to mice (V. W. Yuen et al., Using mouse liver cancer models based on somatic genome editing to predict immune checkpoint inhibitor responses. J Hepatol 78, 376-389 (2023), to generate various spontaneous hepatocellular carcinoma (HCC) models with specific tumor suppressor deficiencies that mimic different mutational subtypes of human HCC.

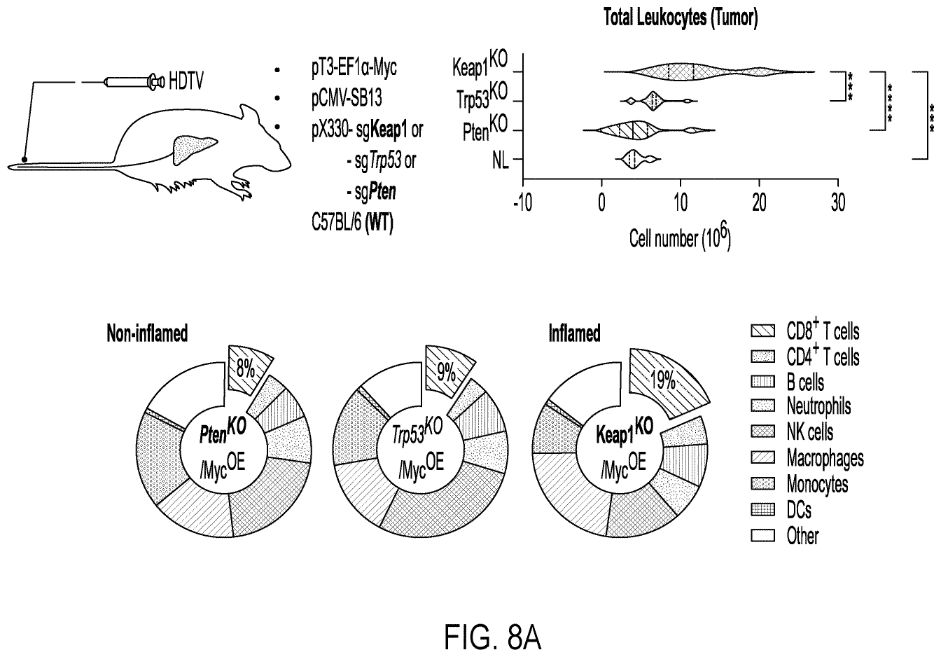

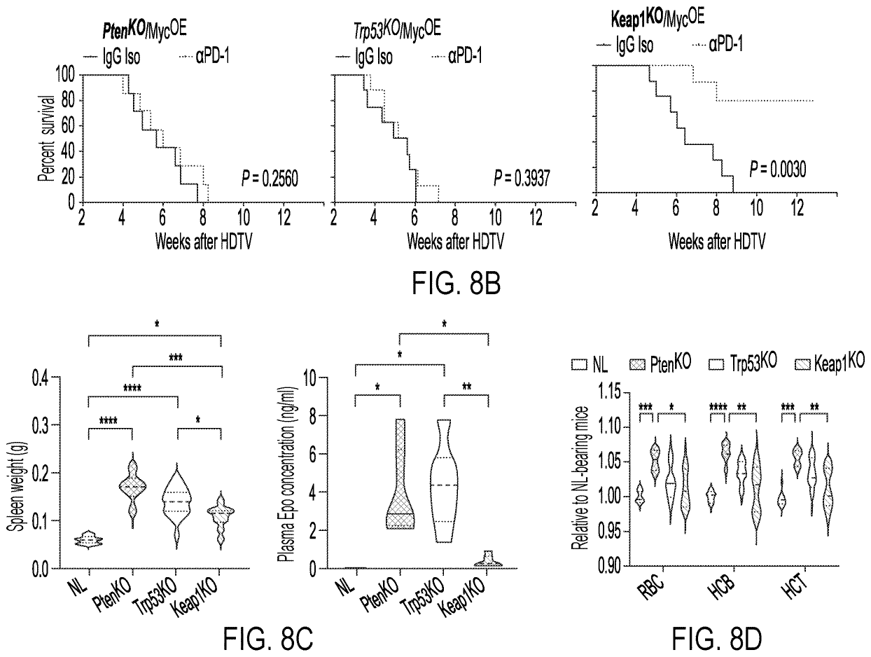

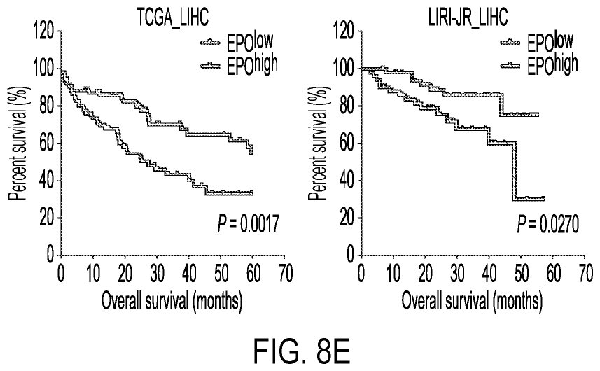

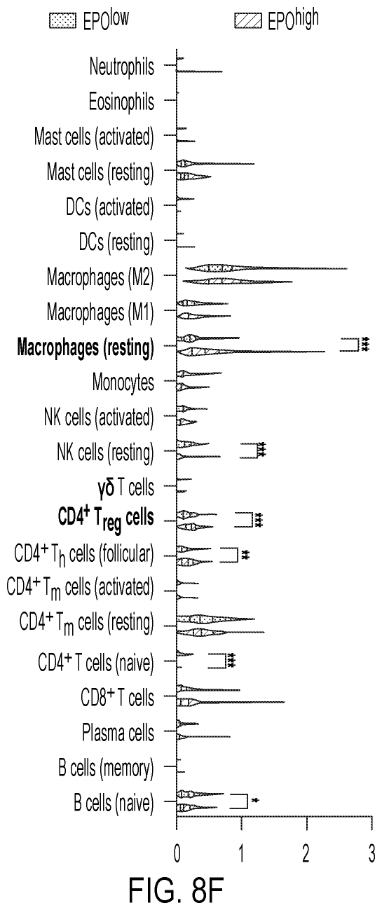

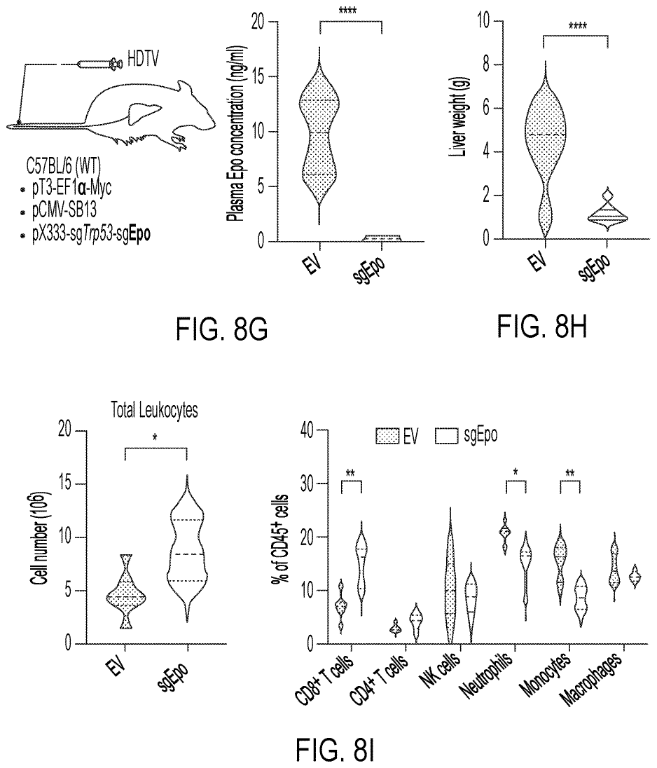

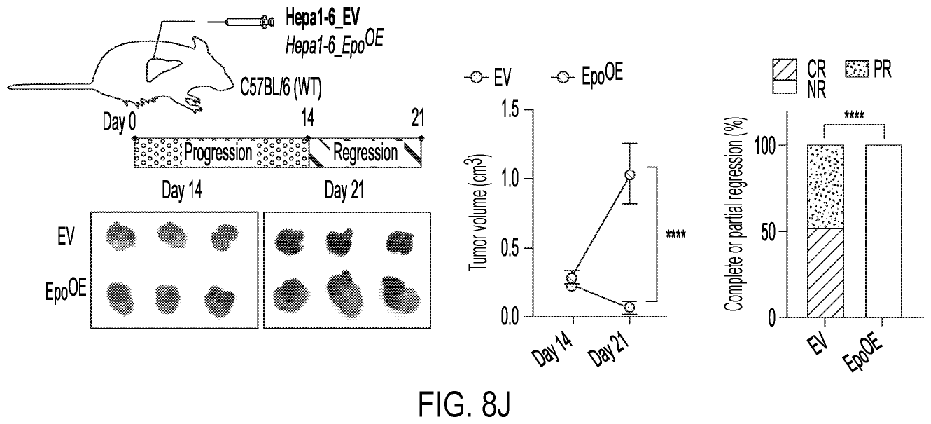

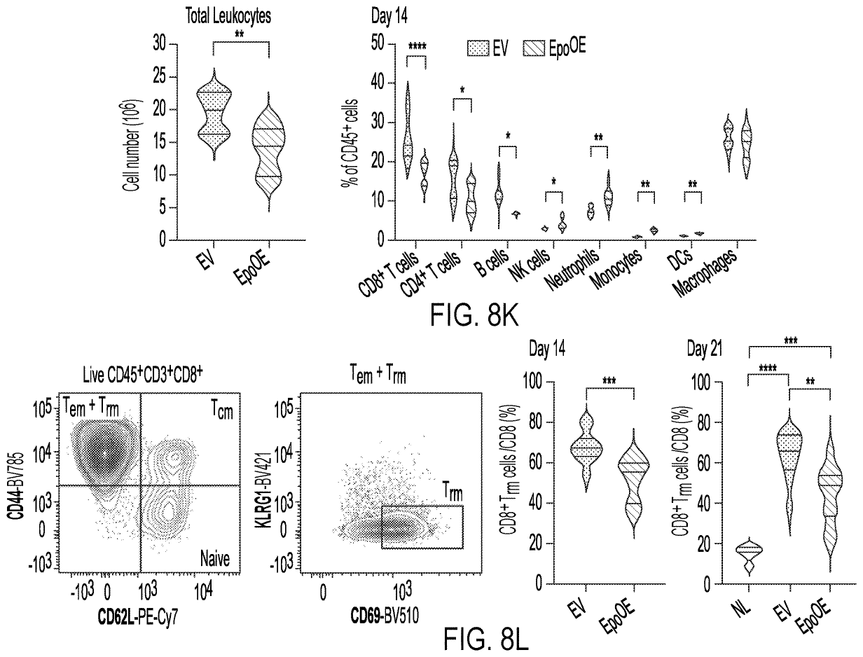

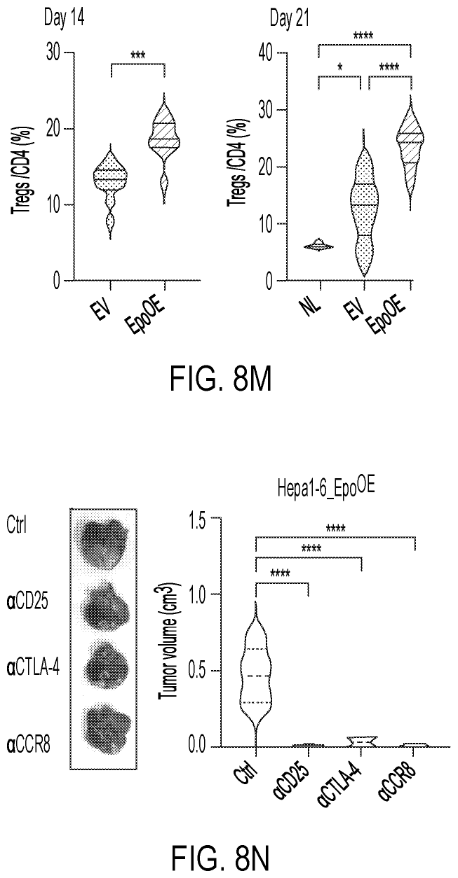

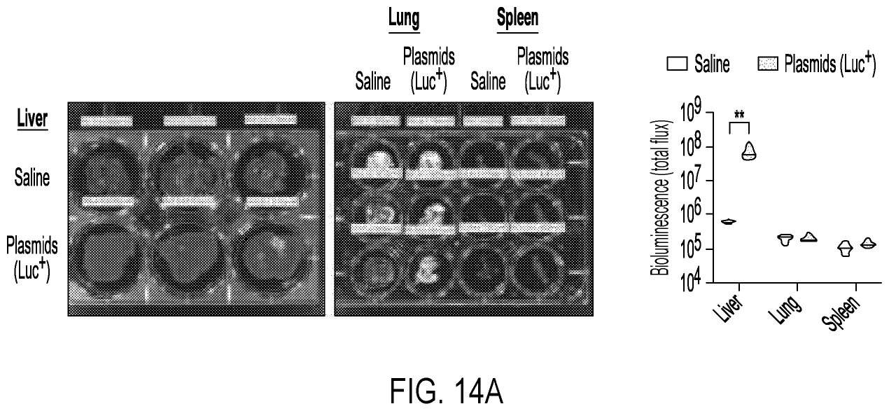

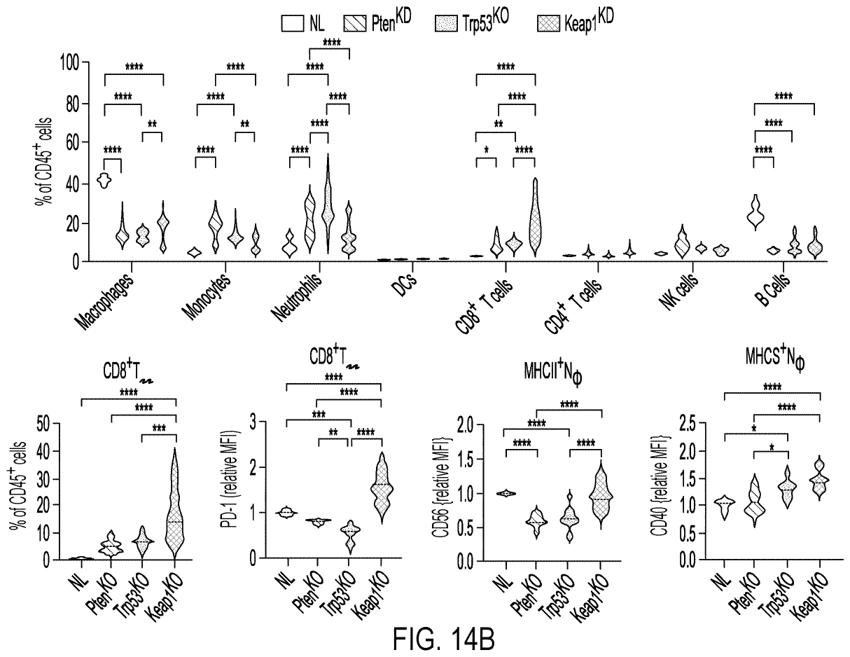

By recapitulating these subtypes, including their immunotypes and responses to immune checkpoint blockade (ICB), this model created a robust framework for investigating the development of the tumor immune landscape. In accord with their human HCC counterparts, Trp53KO and PtenKO tumors exhibited the traits associated with “non-inflamed” tumors, including low infiltration of immune cells, impaired activation and accumulation of CD8+ T cells, as well as resistance to ICB. By contrast, “inflamed” Keap1KO tumors were characterized by an abundance of tumor-infiltrating CD8+ effector memory T cells (TEM) and were highly responsive to ICB (FIGS. 8A-8B and FIG. 14). FIG. 8A shows generation of the spontaneous HCC model by in vivo delivery of plasmids pCMV-SB13, pT3-EF1a-Myc and pX330-sgRNA targeting Trp53, Pten, Keap1 to mouse liver via HDTV. Table 2 provides sequences of gRNAs used. Five weeks after HDTV, HCC tumors were harvested and the immune cell compositions were determined by flow cytometry (n=8-10/group). FIG. 8B provides data for C57BL/6 WT 2 weeks after HDTV, with intraperitoneally (IP) with 2 mg/kg of αPD-1 (RMP1-14) or IgG Isotype control every 3 days (total 5 doses). Overall survival was measured (n=7-9/group) (FIG. 8B).

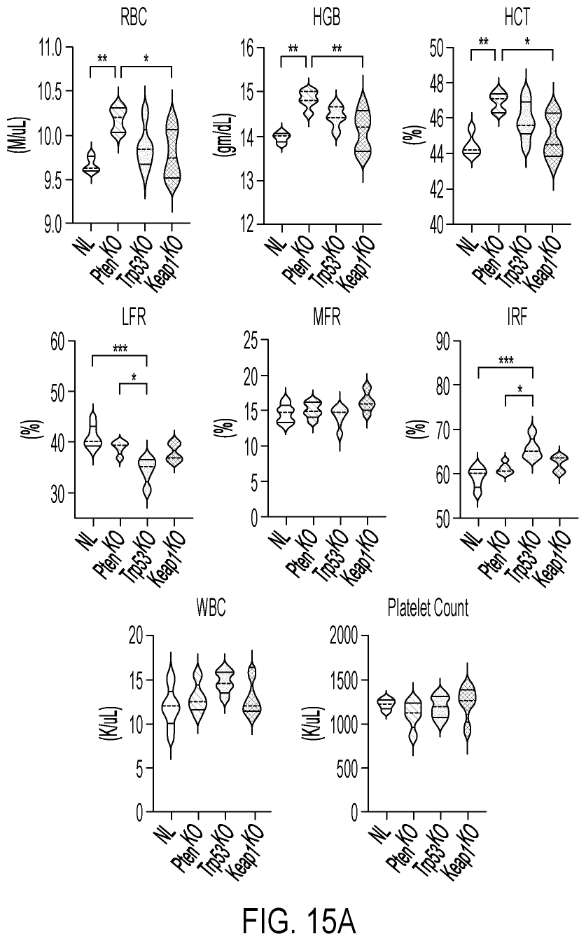

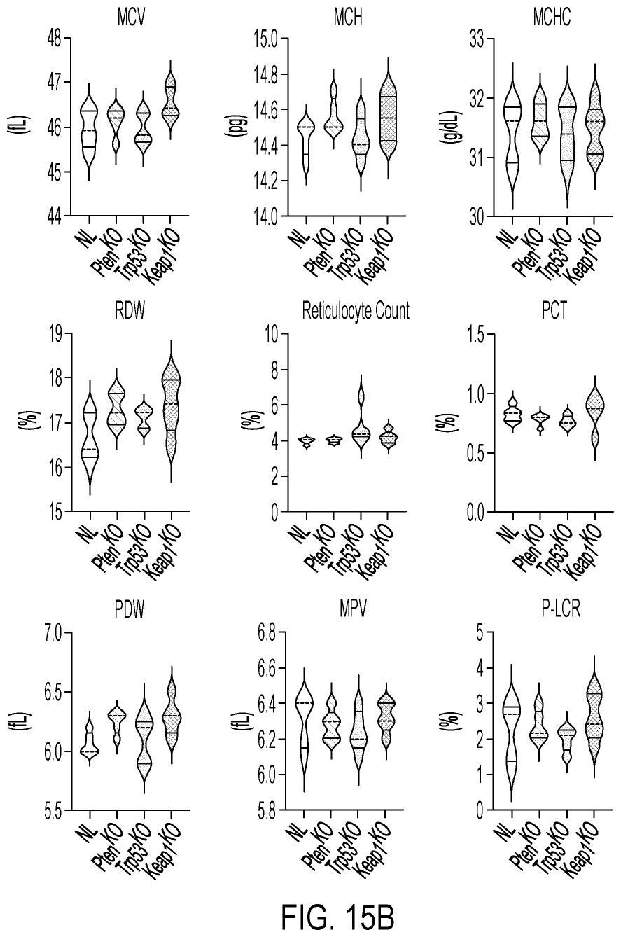

FIG. 8C provides spleen weight and plasma EPO concentration of tumor-bearing mice 5 weeks after HDTV (n=8-10/group). Unexpectedly, splenomegaly and elevation of plasma EPO were exclusively observed in both non-inflamed tumor models (FIG. 8C), even in the absence of anemia (FIG. 15) and regardless of tumor size. FIG. 8D provides data for blood samples from tumor-bearing mice with comparable tumor burdens 4 weeks after HDTV, and analyzed for complete blood count (n=5/group). The levels of RBC, HGB, and HCT are presented. Hemoglobin (HGB), hematocrit (HCT) and red blood cell count (RBC) levels were slightly increased in mice with non-inflamed tumors, likely reflecting the erythropoietic effect of EPO (FIG. 8D). This raises the possibility that EPO, independent of tumor-associated anemia, may act as a mediator in creating an immunosuppressive or non-inflamed TME.

EPO, a glycoprotein hormone recognized for its role in stimulating red blood cell production, can mediate other biological functions. For instance, EPO can promote efferocytosis in macrophages, a process of ingesting apoptotic cells and debris without triggering an immune response against self-antigens. This is vital for preventing excessive inflammation and autoimmune disease.

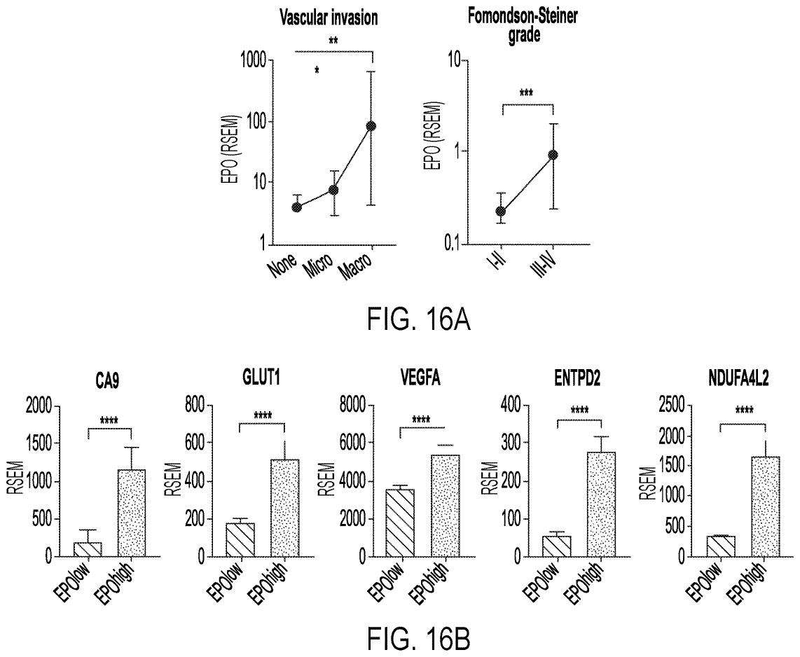

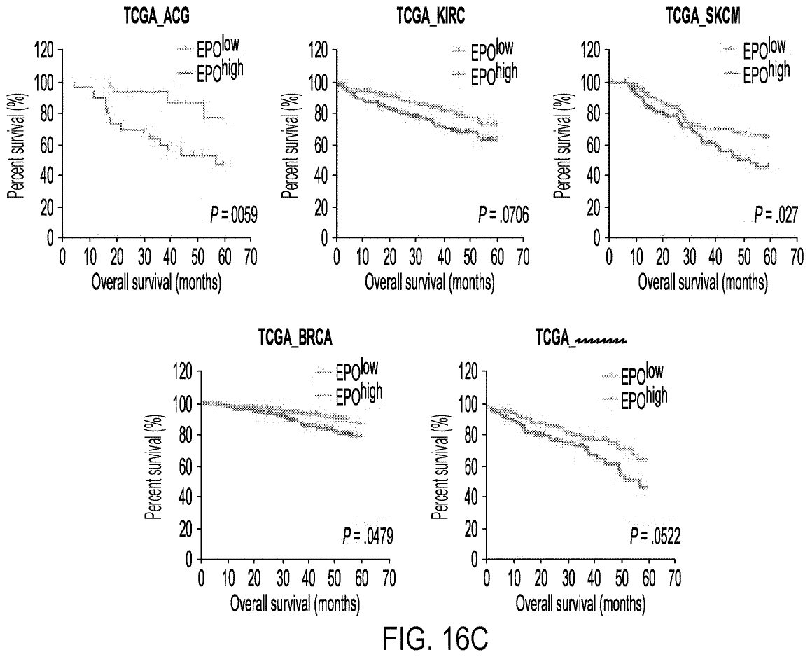

Analysis of human HCC samples from The Cancer Genome Atlas (TCGA) and ICGC International Cancer Genome Consortium (ICGC) databases revealed that elevated EPO expression in tumors was associated with worsened survival, macrovascular invasion and poorer tumor differentiation by Edmondson-Steiner grading (FIG. 8E and FIG. 16A). The elevated production of EPO in HCC patients may be attributed to the activation of hypoxia-inducible factors (HIFs), as evidenced by a positive correlation between EPO levels and common hypoxia-inducible genes identified in HCC (FIG. 16B). Digital cytometry by CIBERSORTx further estimated that EPO overexpression correlated with higher frequencies of Tregs and M0 resting macrophages within the TME of HCC (FIG. 8F). Emerging evidence suggests that the M0 resting macrophages identified by CIBERSORTx represent an immunoregulatory macrophage population linked to poor prognosis in several cancers, including HCC. Beyond HCC, EPO is also produced by pancreatic ductile adenocarcinoma and prostate cancer, and analysis of the TCGA revealed that high EPO expression was associated with a poorer prognosis in other cancers such as kidney, breast, colorectal, and skin cancers (FIG. 16C).

### Example 12—Tumor-Derived EPO Autonomously Promotes an Immunosuppressive Tumor Microenvironment (TME)

To directly examine the role of tumor-derived EPO in shaping the TME, Trp53^{KO} HCC tumors that were unable to produce EPO, were induced via HDTV delivery of a transient CRISPR-Cas9 vector that co-expresses two sgRNAs (pX33-sgTrp53-sgEpo). Table 2 provides sequences of the gRNAs used. The pX333 vector has two independent U6 promoters, which allow for the expression of two sgRNAs. pX333-sgTrp53-sgEpo was generated to render Trp53^{KO} tumors unable to secrete Epo.

FIG. 8G provides data for blood samples collected from tumor-bearing mice with comparable tumor burdens and measured for plasma EPO concentration 4 weeks after HDTV (n=6/group). Ablation of EPO in these non-inflamed tumors effectively abrogated the previously observed elevation of plasma EPO levels, suggesting that non-inflamed tumors are the primary source of EPO production in this model (FIG. 8G).

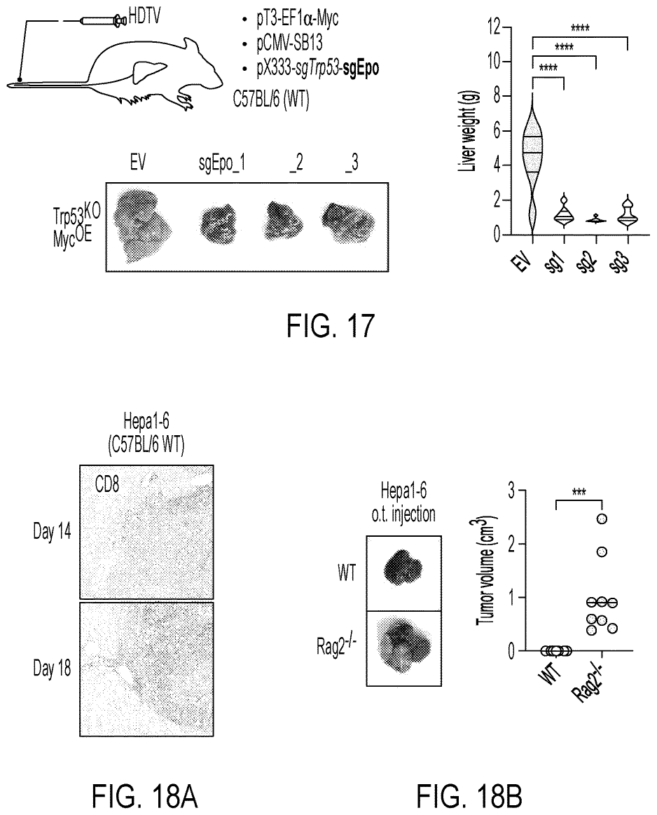

FIG. 8H shows Trp53^{KO}/Myc^{OE} and Trp53^{KO}/Epo^{KO}/Myc^{OE} HCC tumors harvested for size measurement 5 weeks after HDTV. EPO ablation dramatically diminished both the tumor burden and incidence rate (FIG. 8H and FIG. 17). This was accompanied by a marked increase in total leukocytes and effector CD8+ T cells (FIG. 8I), alongside a reduction in neutrophils and monocytes, indicating that EPO plays a critical role in promoting non-inflamed tumor development in HCC.

A tumor model in was generated by orthotopically implanting C57BL/6 mice with 3×10⁶ allogeneic Hepa1-6 cells, derived from the C57L strain. The Hepa1-6 tumors exhibited growth for 14 days followed by subsequent spontaneous regression including complete tumor clearance in almost half the mice. FIG. 8J shows Hepa1-6_EV (empty vector) and Hepa1-6_Epo^{OE} (EPO-overexpressing) tumors harvested on Day 14 and Day 21 (n=8-12/group) for determination of tumor size and regression rate (CR: complete regression; PR: partial regression; NR: no regression). This regression was attributed to T cell immunity, as massive CD8+ T cell infiltration was observed in tumors in wild type C57BL/6 mice, while the tumors did not regress in Rag2-deficient mice on the same genetic background (FIG. 8J and FIGS. 18A-18B), underlining the crucial role of T cells in tumor control. Notably, Hepa1-6 tumors that overexpressed EPO (Hepa1-6_Epo^{OB}) escaped regression and continued to grow, accompanied by a reduction in total leukocytes and a shift in the TME from T cell-rich to myeloid cell-rich (FIGS. 8J-8K). Furthermore, Hepa1-6_Epo^{OE} tumors displayed a lower proportion of activated CD8+CD69+ tissue-resident memory T cells (Trm) but a higher proportion of CD4+ regulatory T cells (Treg) (FIGS. 8L-8M).

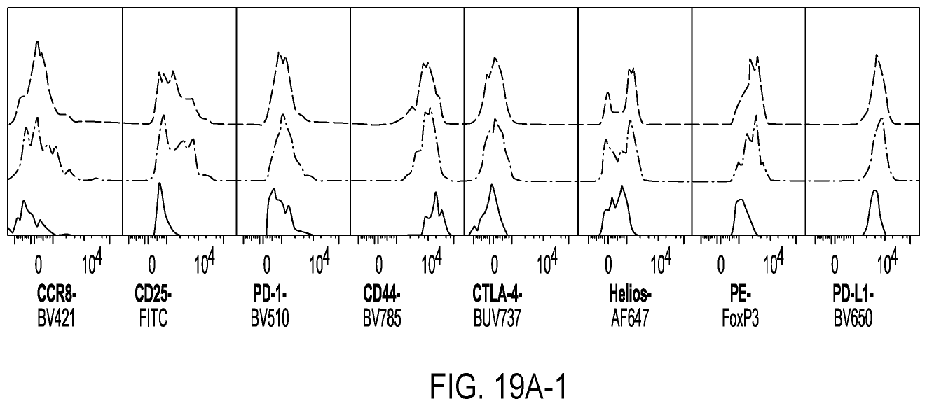

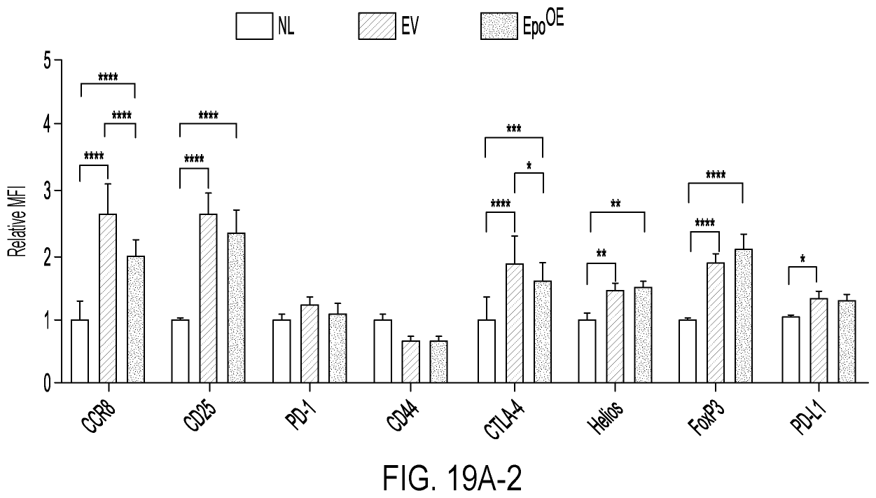

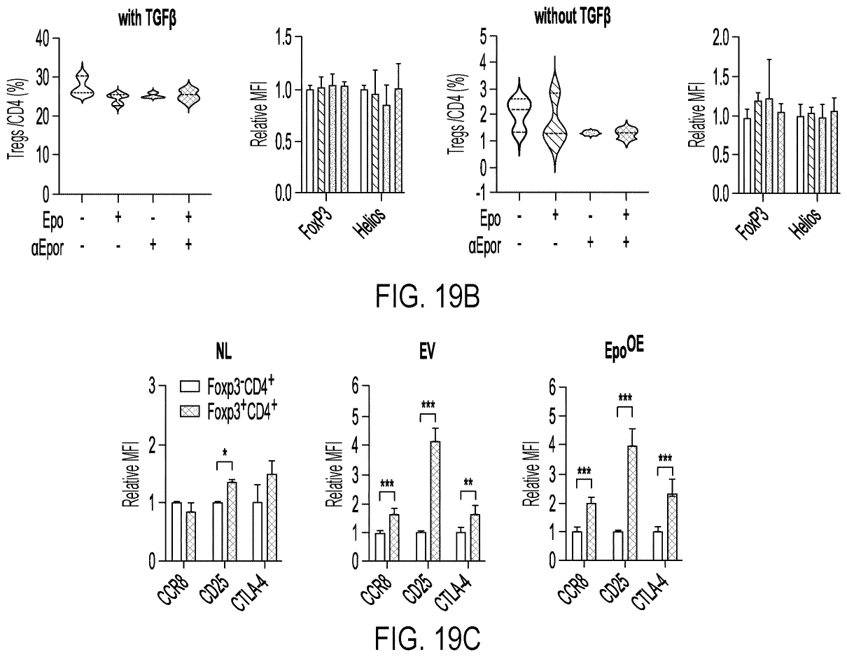

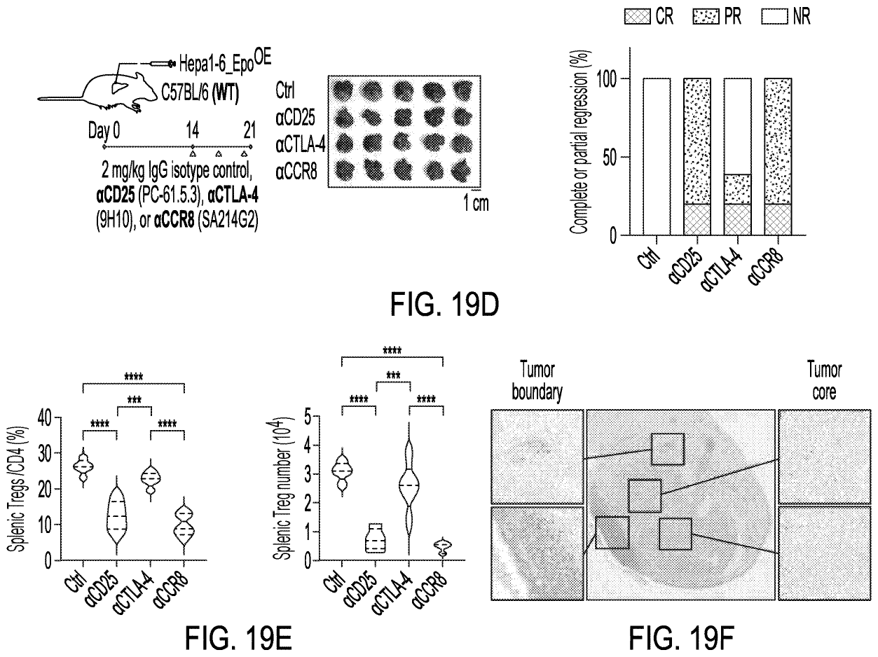

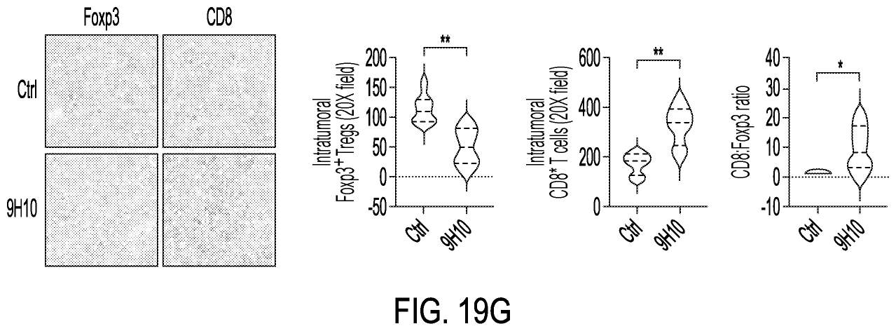

While EPO did not directly influence Treg polarization or activation, the higher Treg accumulation in Hepa1-6_Epo^{OE} tumors suggested an indirect mechanism (FIGS. 19A-19B). Tumor-infiltrating Tregs in this model preferentially expressed CCR8 and CTLA-4 (FIG. 19C).

On days 14, 17 and 20 after orthotopic implantation (Hepa1-6_Epo^{OE}), mice were injected IP with 2 mg/kg of αCD25, αCTLA-4, αCCR8 or IgG control. Tumors were harvested on day 21 (n=5/group). Depleting Tregs using anti-CTLA4, anti-CCR8, or anti-CD25 antibodies beginning 14 days after tumor implantation led to the regression of Hepa1-6_Epo^{OE} tumors (FIG. 8N and FIGS. 19D-19G). These results demonstrate that Tregs contribute to EPO-mediated immune suppression. Taken together, based on both loss-of-function and gain-of-function experiments, these data demonstrate that EPO autonomously shapes the immune compartment within the TME, thereby facilitating evasion from T cell-mediated tumor attack.

### Example 13—Immunoregulatory Macrophages are the Predominant EPOR+ Populations in Human and Mouse HCC

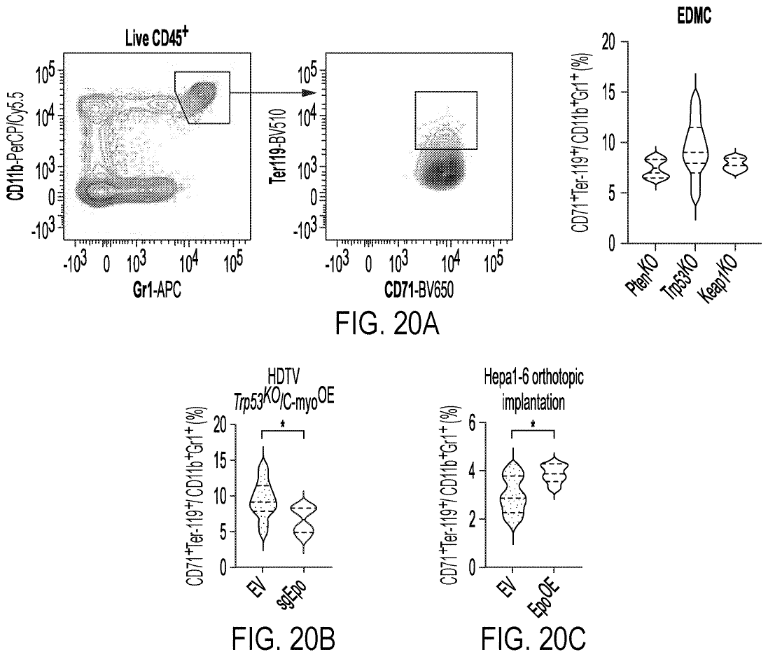

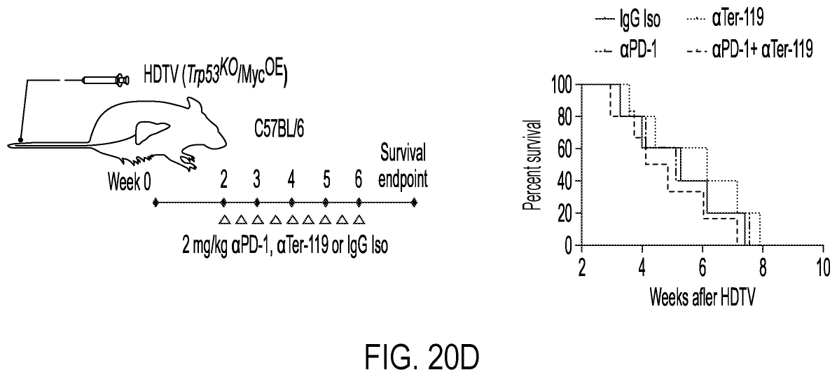

EPO stimulates erythroid progenitors to induce tumor-promoting erythroid-differentiated myeloid cells (EDMCs). In the spontaneous HCC models disclosed herein, no significant difference in the proportion of EDMCs between inflamed and non-inflamed tumors were observed (FIG. 20A), suggesting that EDMCs are not a critical factor shaping the tumor microenvironment. Ablation of EPO secretion by tumor cells reduced EDMC accumulation, while EPO overexpression had the opposite effect (FIGS. 20B-20C), indicating that EPO may contribute to EDMC accumulation. However, depletion of EDMCs or erythroid progenitors did not improve survival in non-inflamed HCC-bearing mice or enhance their responsiveness to PD-1 blockade therapy (FIG. 20D), suggesting that the immunomodulatory effect of EPO on the TME was not primarily mediated by EDMCs.

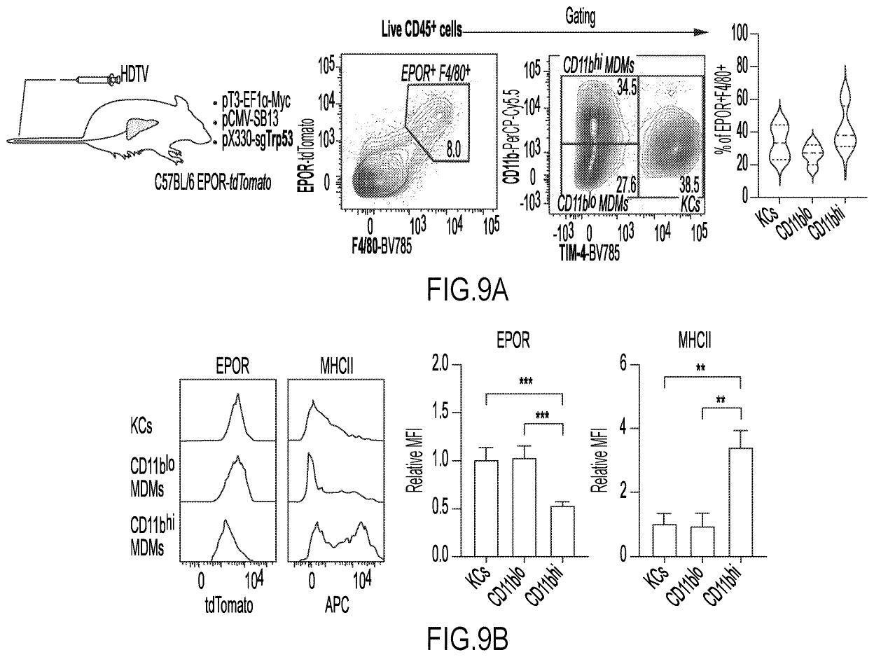

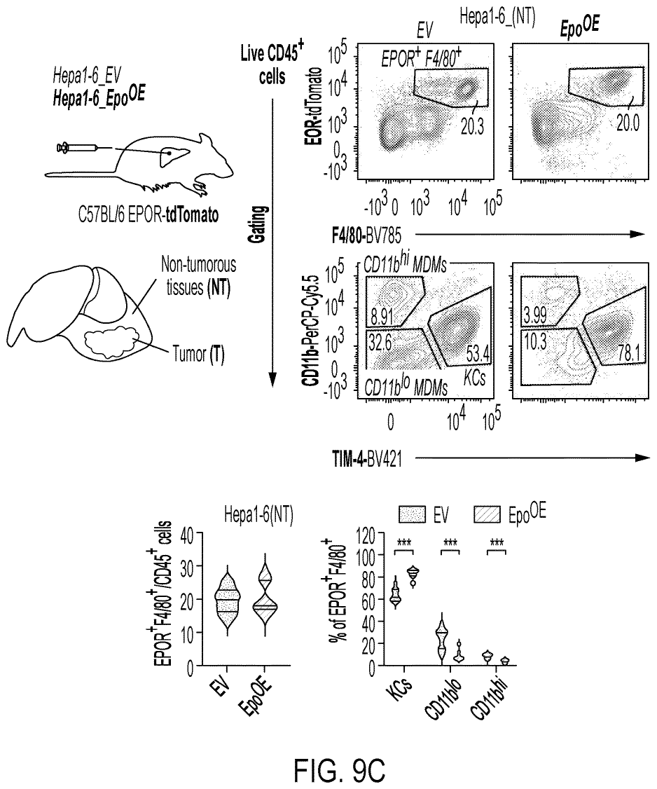

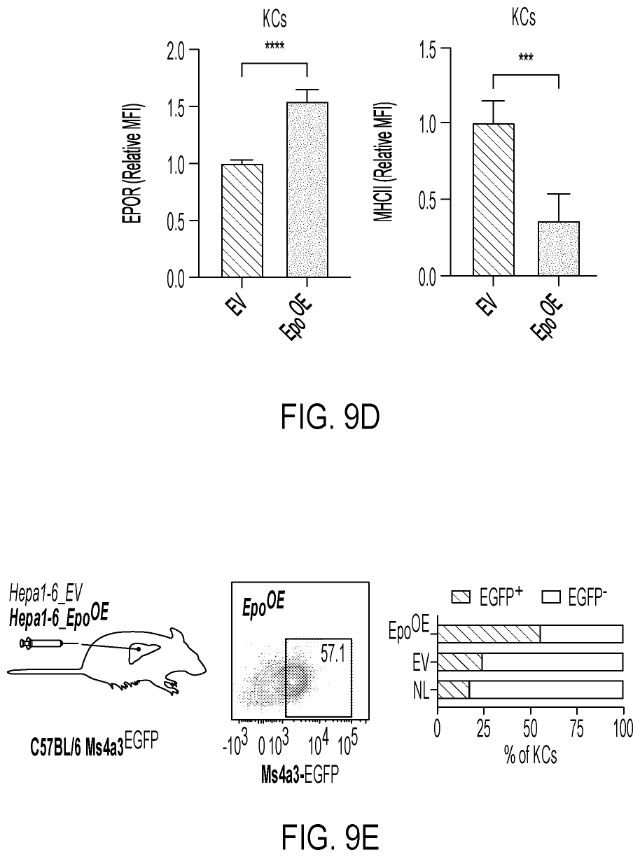

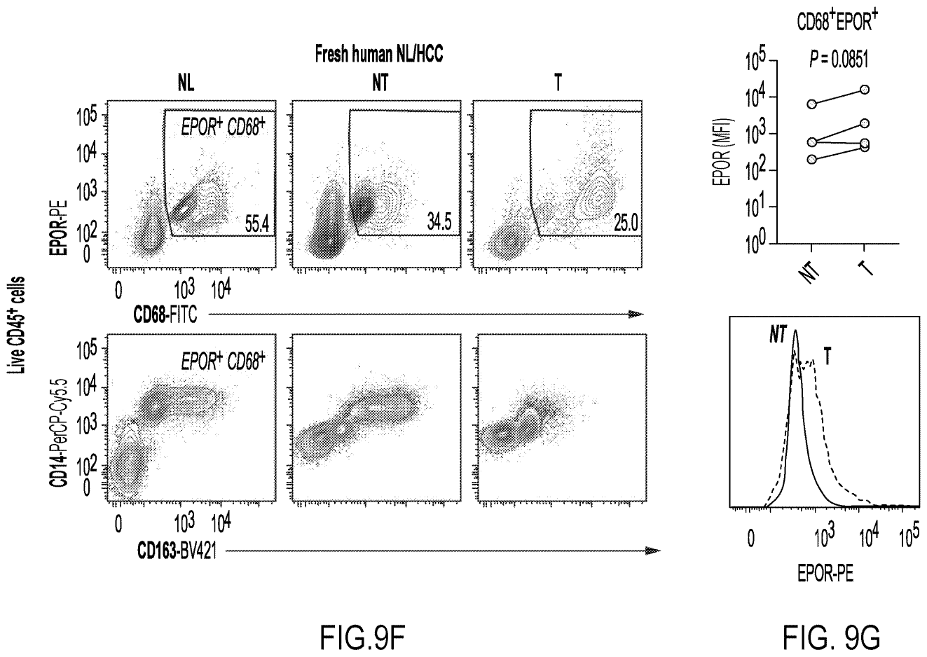

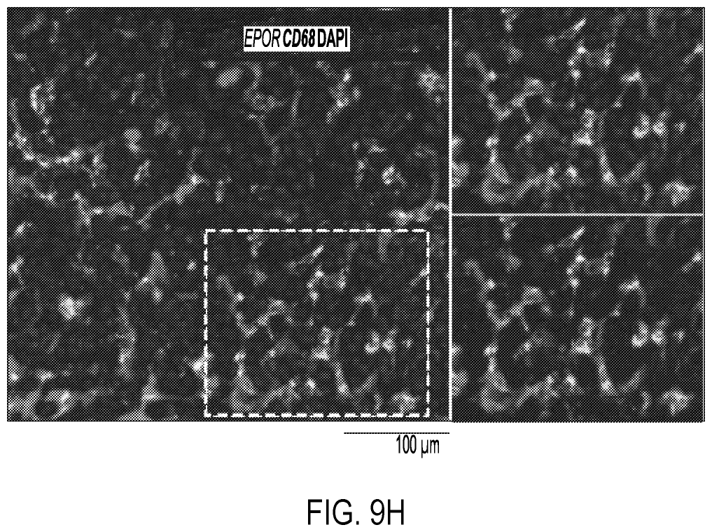

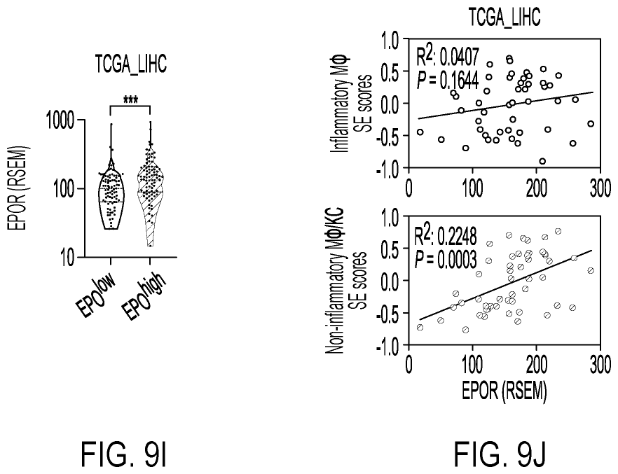

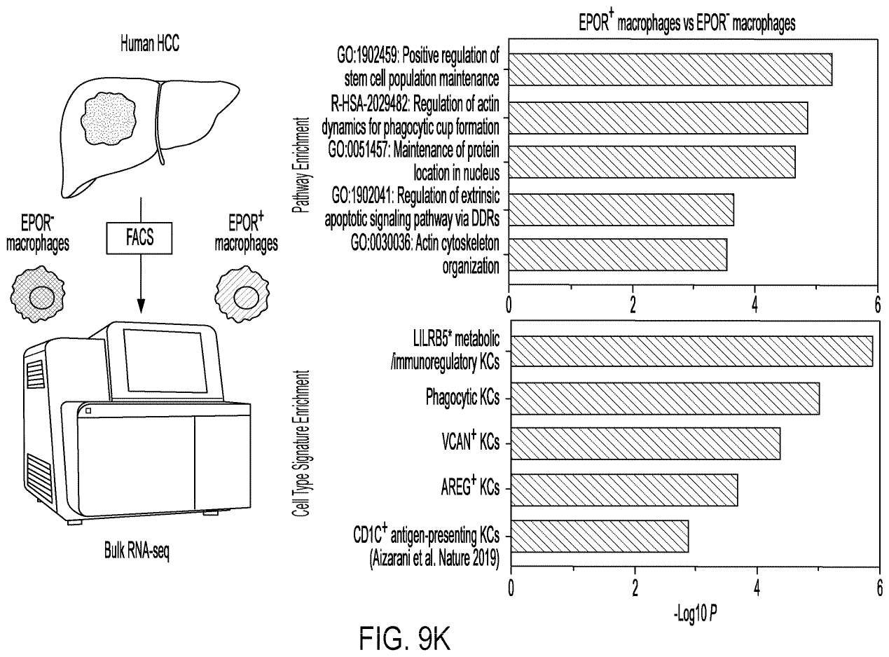

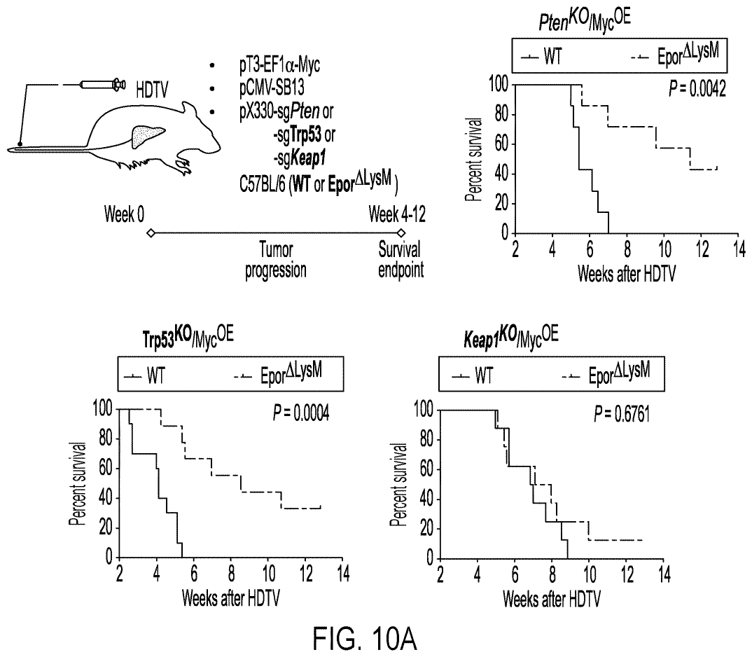

Because EPOR+ immune cells may account for the immune suppression instigated by EPO, the immune cell populations that express EPOR in HCC were identified. Studies of “non-inflamed” Trp53KO HCC in EPOR-tdTomato reporter mice revealed that tumor-infiltrating EPOR+ leukocytes consisted mainly of F4/80+ macrophages, comprising three distinct subsets: CD11b^{hi} monocyte-derived macrophages (MDMs), CD11b^{lo} MDMs, and TIM-4+ macrophages (FIG. 9A). TIM-4 serves as a marker for Kupffer cells (KCs), which are liver-resident macrophages primarily originating from yolk-sac macrophages. In adults, monocytes can migrate from the bone marrow to the liver, giving rise to a minor proportion of KCs. Regardless of their origin, TIM-4+ macrophages were assessed as KCs.

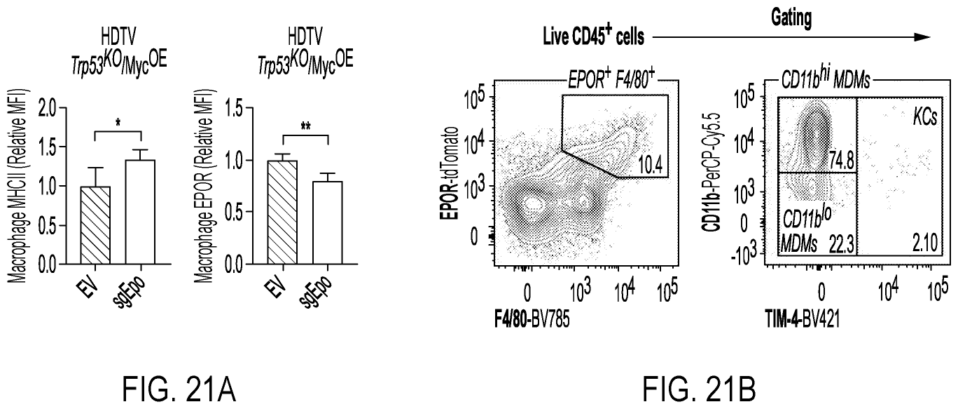

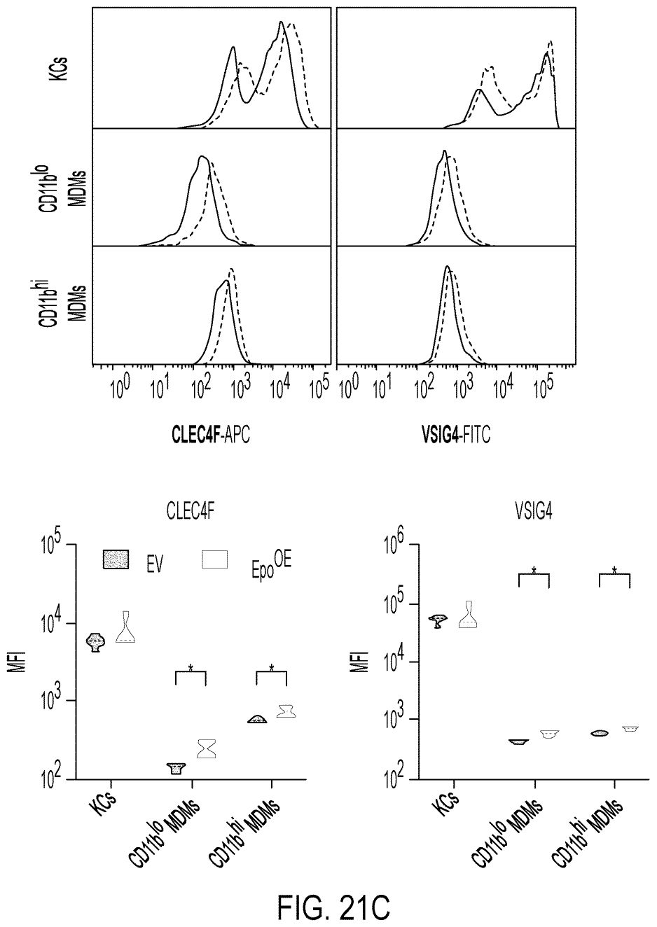

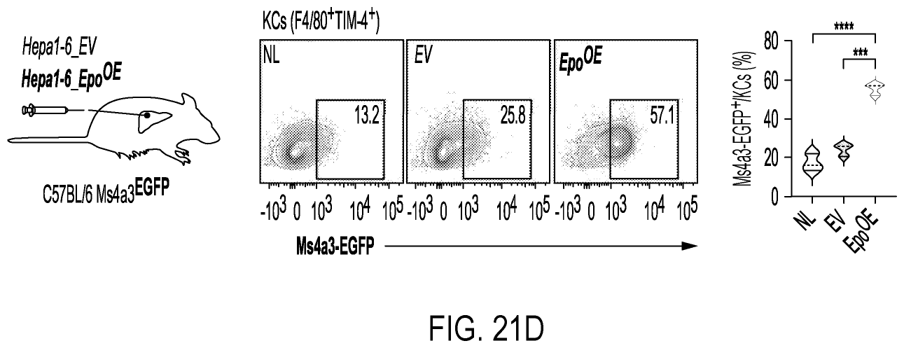

Among the three EPOR+ macrophage subsets, CD11b^{lo} MDMs and KCs exhibited higher EPOR expression but lower MHCII levels (FIG. 9B). Ablation of EPO resulted in increased EPOR expression but reduced MHCII levels on macrophages (FIG. 21A). TIM-4+ KCs were seldom observed in “inflamed” Keap1KO HCC, implying a potential role for these cells in EPO-induced suppression (FIG. 21B).

Two weeks after orthotopic implantation (Hepa1-6_EV or Hepa1-6_Epo^{OE}), liver tissues of EPOR-tdTomato reporter mice were harvested and subjected to flow cytometry analysis. While EPO derived from Hepa1-6_Epo^{OE} tumors did not alter the overall frequency of EPOR+ macrophages in liver, EPO expanded the TIM-4+ KC populations (FIG. 9C). The identity of KCs was further confirmed by their expression of CLEC4F and VSIG4 (FIG. 21C). Additionally, EPO upregulated the levels of EPOR and downregulated the levels of MHCII on KCs (FIG. 9D).

Two weeks after orthotopic implantation (Hepa1-6_EV or Hepa1-6_Epo^{OB}), liver tissues of Ms4a3-EGFP reporter mice were harvested and the proportion of EGFP⁺ cells in KCs was determined. Using Ms4a3-EGFP reporter mice to trace monocyte-derived cells, it was observed that while normal livers contained minimal EGFP+ KCs, EPO expression in tumors led to a dramatic increase in the proportion of EGFP+ KCs. This suggests that the EPO-driven expansion of KCs or KC-like cells may arise from the differentiation of monocytes or MDMs, rather than local proliferation of embryonic-derived KCs (FIG. 9E and FIG. 21D).

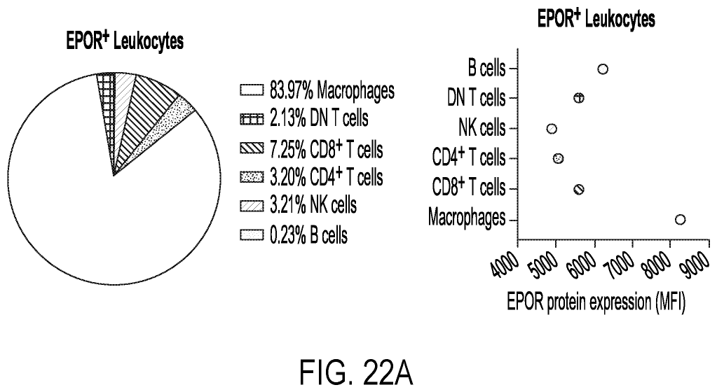

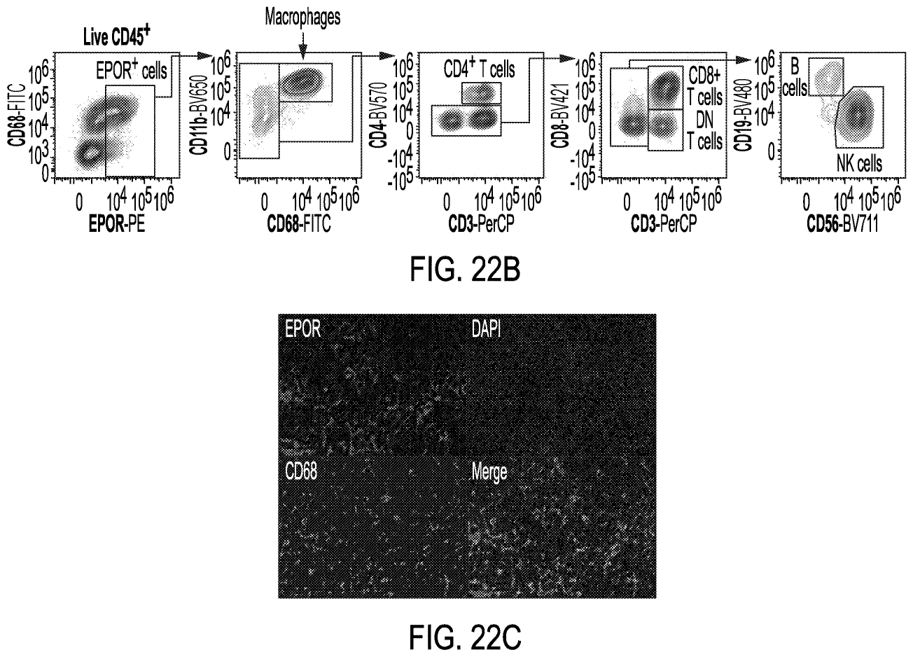

Fresh normal liver tissues were procured from deceased organ donors, while fresh tumors (T) and adjacent non-tumor liver tissues (NT) were obtained from surgical resection of HCC patients. Within 24 hours post-collection, samples were dissociated into single-cell suspensions. Studies of human tissues confirmed the presence of EPOR+ cells in normal liver and HCC tumor tissues (T), as well as in non-tumorous tissues (NT) adjacent to the tumors, by analyzing leukocytes from fresh tissues using flow cytometry and immunofluorescence imaging. The EPOR+ cells were predominantly CD11b+CD68+ macrophages, with a minor contribution from lymphocytes exhibiting low EPOR expression in the liver. The EPOR expression of macrophages showed a trend toward being higher in T compared to NT (FIGS. 9F-9H and FIGS. 22A-22C).

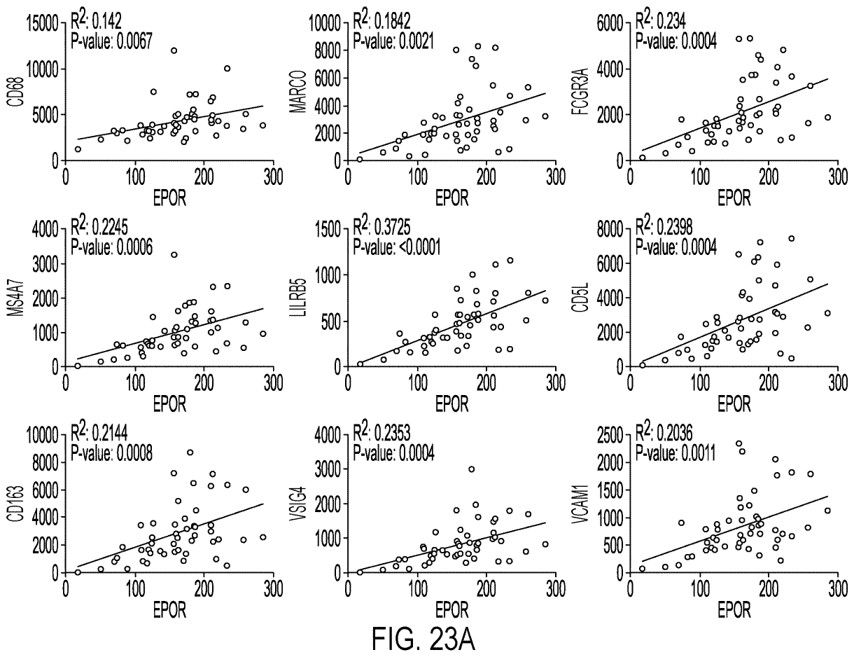

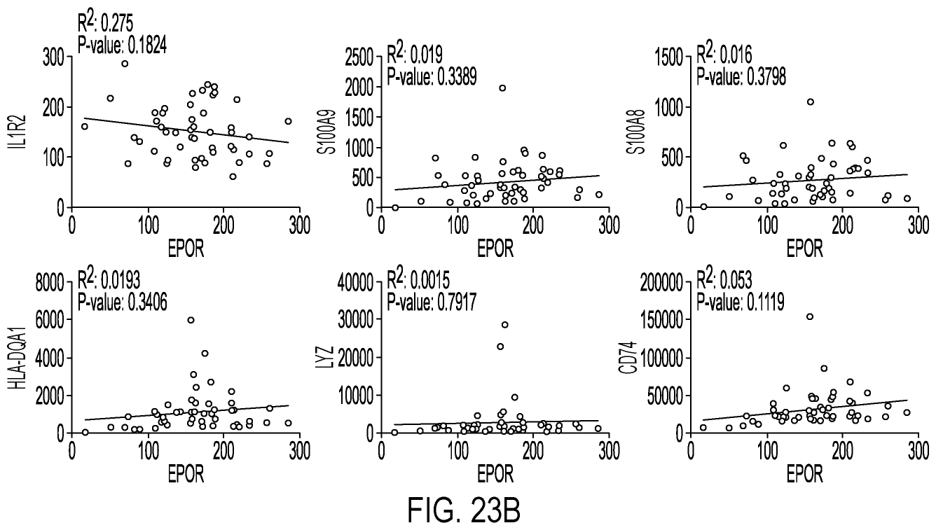

Overexpression of EPO mRNA also correlated with higher EPOR mRNA levels in human HCC (FIG. 9I). There are two distinct populations of macrophages, non-inflammatory KC-like macrophages and inflammatory macrophages. EPOR expression was positively associated with the signature enrichment scores of non-inflammatory KC-like macrophages (FIG. 9J and FIG. 23).

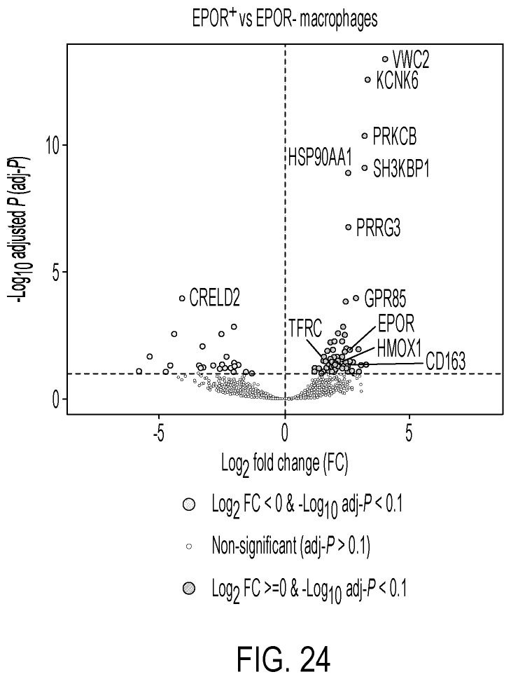

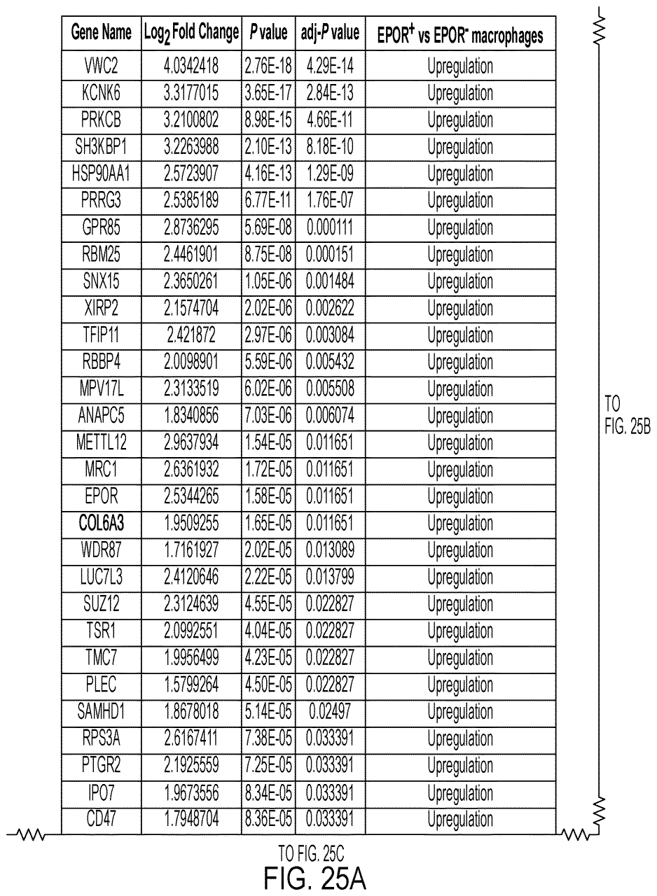

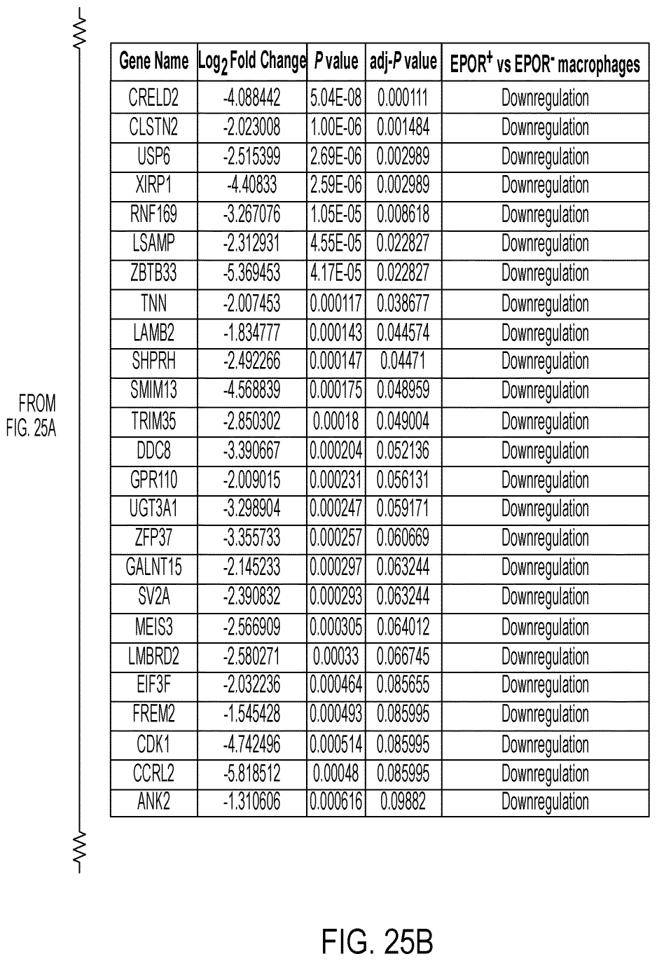

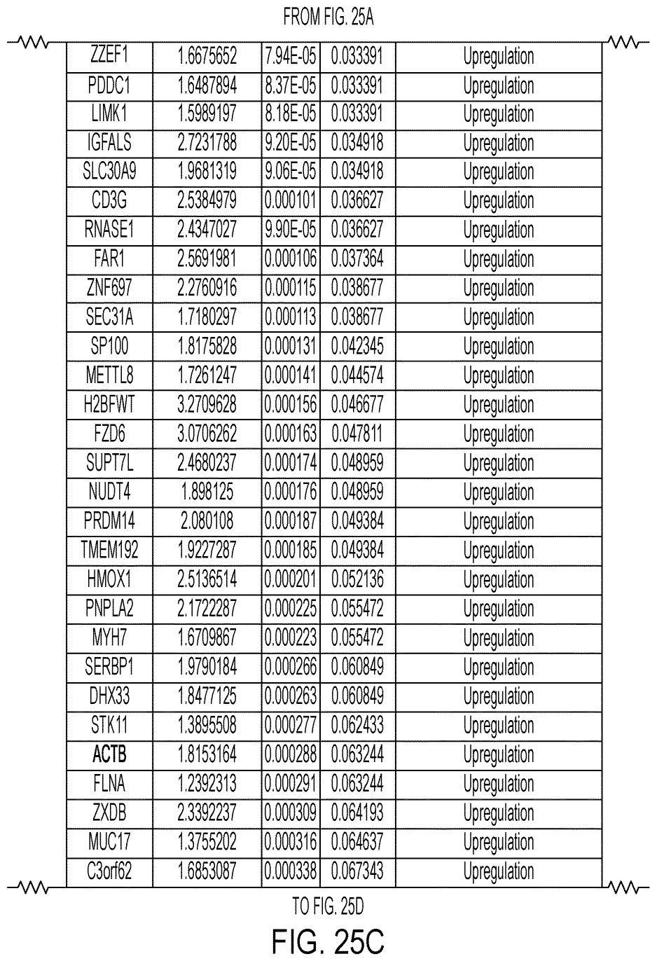

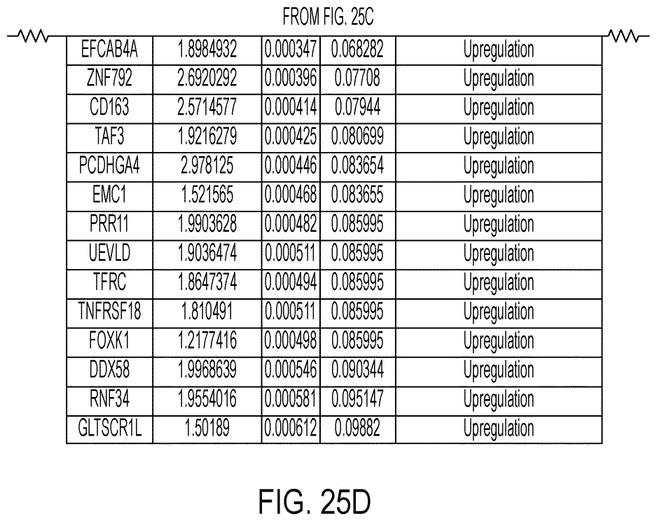

To investigate the nature of EPOR+ macrophages in human HCC, fresh liver tumors from surgical resections of HCC patients were procured. FACS was used to isolate EPOR+ macrophages (CD11b+CD14+) and EPOR-macrophages and subjected them to bulk-RNA sequencing. Seventy-two genes that were significantly enriched in EPOR+ macrophages were found, and these genes were related to the self-renewal and phagocytosis functions of KCs (FIG. 9K and FIGS. 24-25).

Moreover, among the five subsets of KCs, EPOR+ macrophages showed the highest correlation with LILRB5+ metabolic/immunoregulatory KCs (FIG. 9K). These macrophages expressed high levels of molecules such as VSIG4 and HMOX1, which are required for maintaining an intrahepatic tolerogenic and anti-inflammatory environment. This observation aligns with the mouse model data disclosed herein, indicating that macrophages expressing high levels of EPOR tend to display reduced MHCII expression, which is further downregulated by EPO. The data for both mouse and human studies suggests that EPOR+ macrophages, potentially expanded and maintained by EPO, are the major EPOR+ populations in HCC, and have the characteristics of immunoregulatory KCs.

### Example 14—EPO Activation of EPOR+ Macrophages Drives a Non-Inflamed Tumor Immunotype Independent of Genotype and Targeting EPOR Results in Regression of Established Tumors

LysM-driven Epor−/− mice (Epor^{ΔLysM}) were generated, in which mature myeloid cells, predominantly macrophages in liver and HCC, lack EPOR. In this context, the absence of EPOR in macrophages had a profound improvement on the survival of mice with non-inflamed tumors, while having no effect on those with hot tumors that secreted low amounts of EPO (FIG. 10A). In approximately 40-50% of Epor^{ΔLysM} mice (non-inflamed tumor models), no tumors were observed at the study endpoint. To test whether these mice never developed tumors at all or if tumors initially formed but subsequently regressed over time, a modified transposon vector, which simultaneously encoded Myc and luciferase (Myc^{OE}-Luc⁺) was used to monitor tumor growth kinetics via bioluminescence imaging. Tumors began to grow from 2 weeks post-HDTV, and wild-type (WT) mice exhibited linear tumor growth. By contrast, Epor^{ΔLysM} mice displayed an oscillating pattern (FIG. 10B), indicating that in the absence of EPOR⁺ macrophages the tumors developed but were subjected to active immunosurveillance.

Keap1^{KO} tumors also displayed oscillating growth kinetics. Despite secreting significantly lower levels of EPO compared to non-inflamed tumors, administration of human recombinant EPO accelerated the growth of Keap1^{KO} tumors, resulting in linear growth kinetics similar to non-inflamed tumors (FIG. 10C).

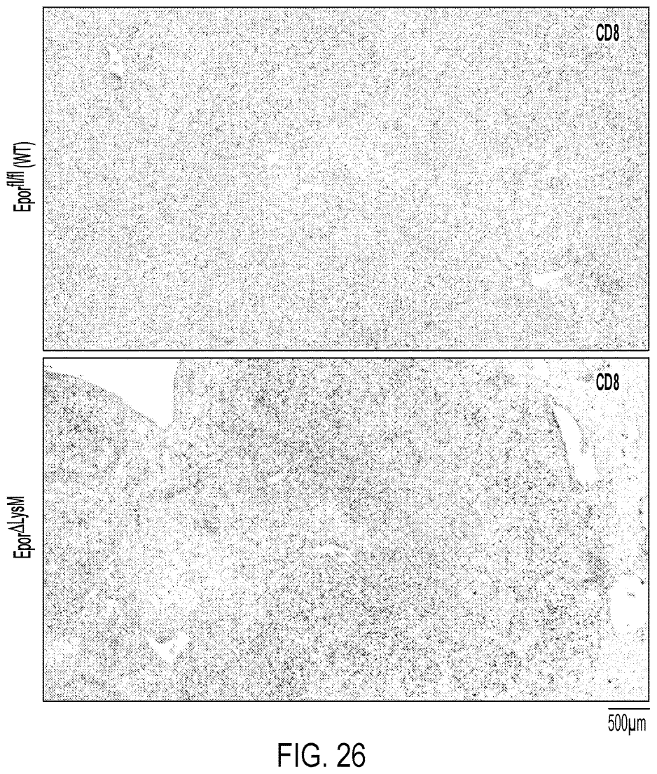

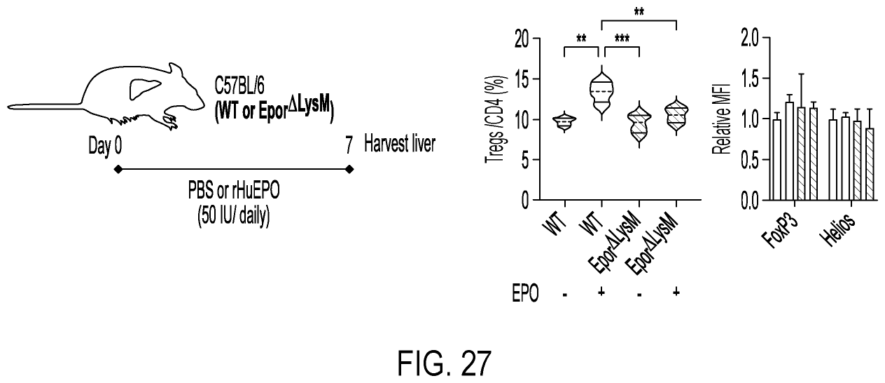

Neither the Epo-secreting tumors in Epor^{ΔLysM} mice nor Epo^{KO} tumors in WT mice could escape immunosurveillance and as a result, they failed to progress (FIG. 10D). These data confirm that tumor-derived EPO and macrophage-expressed EPOR are two indispensable factors for immunosuppression in non-inflamed HCC. Similarly, EPO-overexpressing Hepa1-6 tumors failed to evade T-cell surveillance and regressed in the orthotopic HCC model in which macrophages lacked EPOR (FIG. 10E and FIG. 26). These results demonstrated that this effect is indirect and that EPO-mediated Treg polarization in the liver is dependent on EPO/EPOR signaling in macrophages (FIG. 27). Thus, not only is EPO-activation of EPOR on macrophages required for the development of non-inflamed immunosuppressed tumors, but removal of either one of these molecules results in an inflamed tumor, independent of tumor genotype.

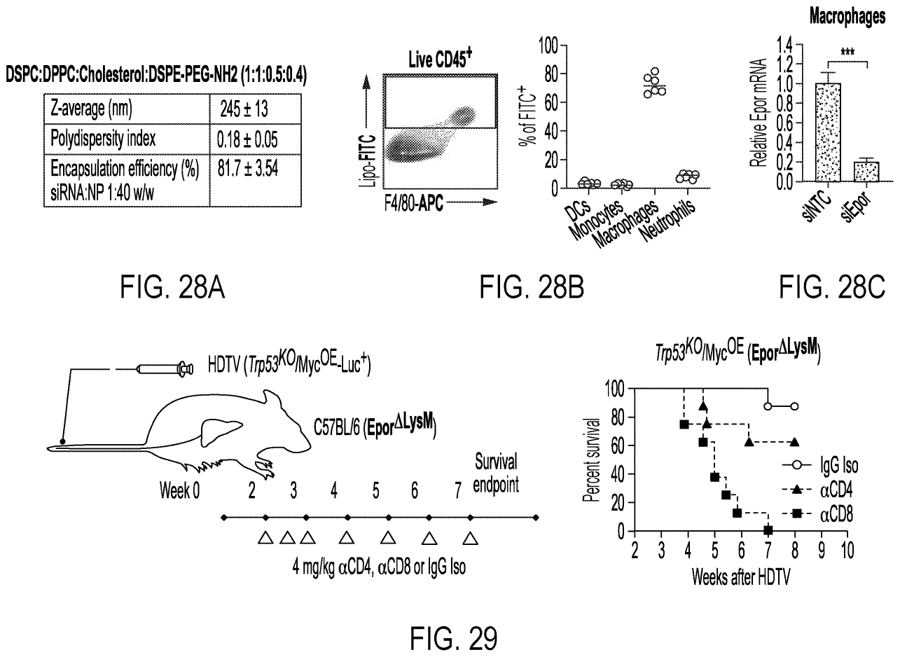

To further validate these results and to determine if inhibition of EPOR expression results in tumor regression after tumors are established, an additional method to reduce EPOR expression in macrophages was employed. Liposomes loaded with siRNA targeting EPOR were administered to tumor-bearing mice and it was confirmed that they were mainly taken up by macrophages (FIG. 28). Liposomes containing 50 μg of siEpor or siNTC (non-targeted control) were administered intravenously (IV) to Hepa1-6_Epo^{OE} (n=6/group) or Trp53^{KO}/Myc^{OE} (n=8/group) HCC bearing mice, and tumor burden was measured. This strategy led to tumor regression similar to that seen in mice with macrophage-specific deletion of EPOR, indicating that established tumors are dependent on EPOR signaling in macrophages and regress when such signaling is diminished (FIGS. 10F-10G). Collectively, these data strongly support the notion that non-inflamed HCC relies on EPO production to evade immune surveillance, with the macrophage EPO/EPOR axis being the primary immunosuppressive mechanism.

### Example 15—Ablation of EPOR in Macrophages Provokes a T-Cell-Mediated Anti-Tumor Immune Response that Synergizes with ICB

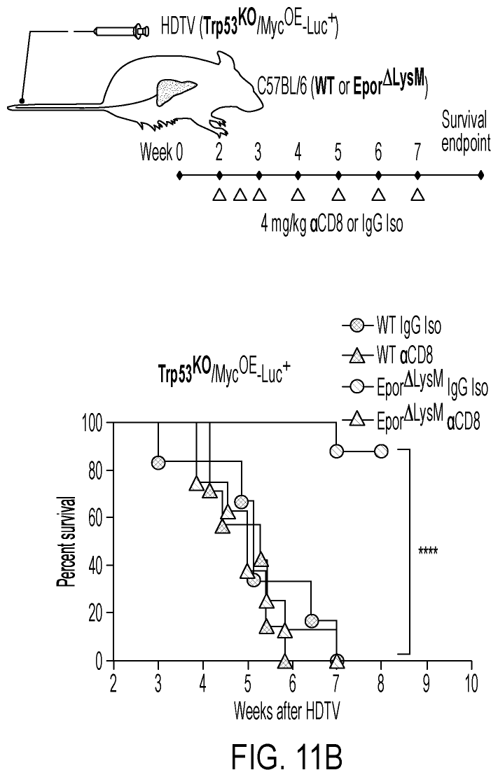

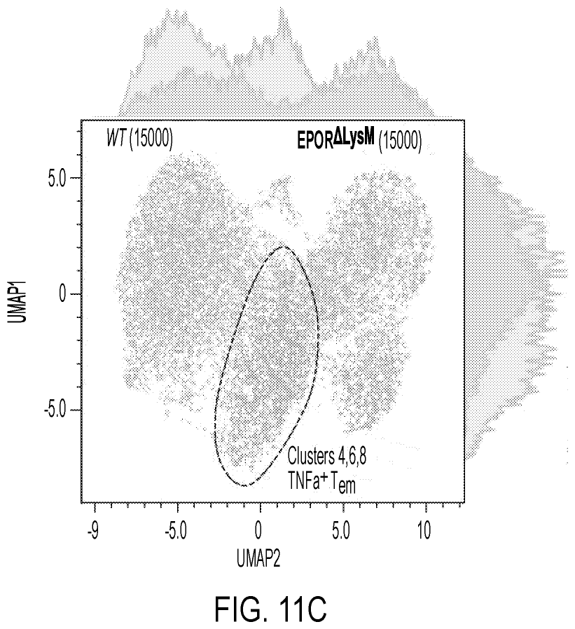

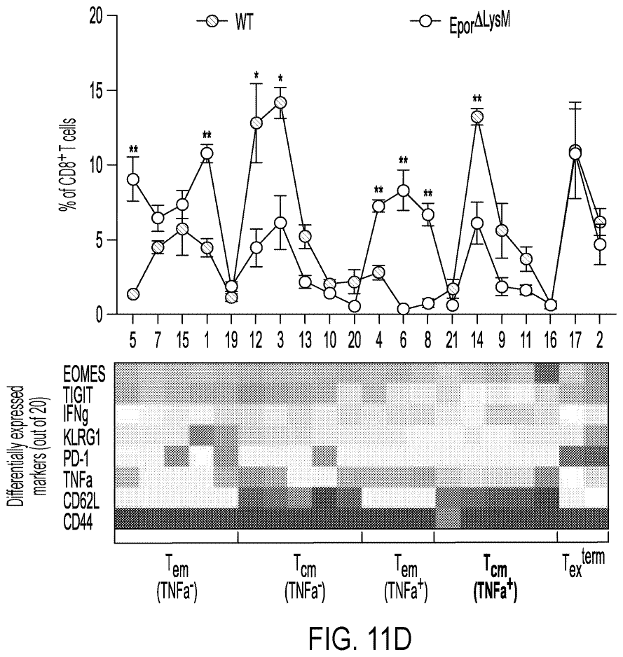

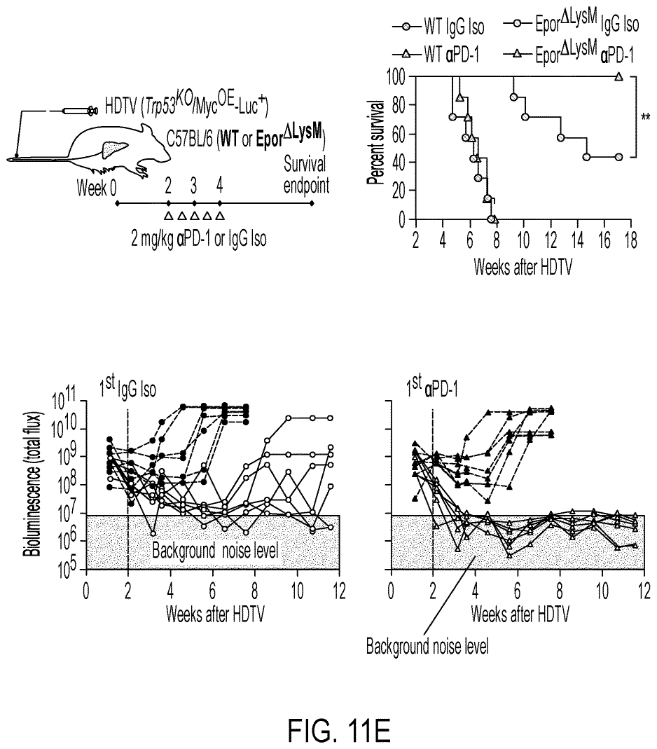

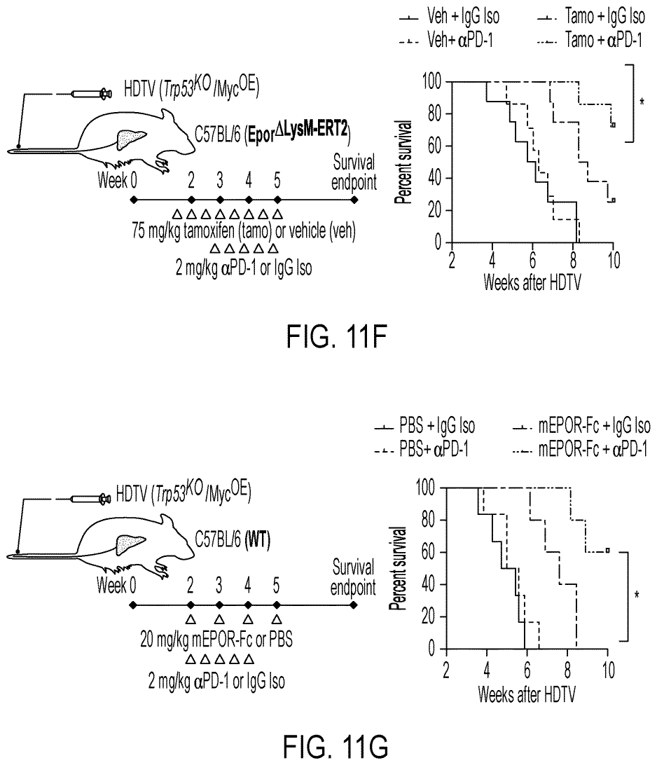

In Trp53^{KO} tumors, knockout of EPOR in macrophages led to substantial increases in absolute leukocyte infiltration and the percentage of effector T cells and macrophages with elevated MHCII expression (FIG. 11A). This implied a transformation from a non-inflamed to an inflamed HCC, supporting that EPOR⁺ macrophages are essential for creating an immunosuppressive TME.

Two weeks after HDTV (Trp53^{KO}/Myc^{OE}), C57BL/6 WT or Epor^{ΔLysM} mice were injected (IP) with 4 mg/kg of an anti-CD8 mAb (YTS 169.4) or IgG control twice during the first week and then once weekly (total 6 doses). Overall survival was measured (n=8/group). While antitumor immunity was restored and some tumors regressed spontaneously in Epor^{ΔLysM} mice, depletion of CD8⁺ T cells dramatically shortened the survival of these mice and there was no sign of regression, indicating that the antitumor immunity conferred by ablation of EPOR in macrophages is CD8⁺ T cell-dependent (FIG. 11B and FIG. 29).

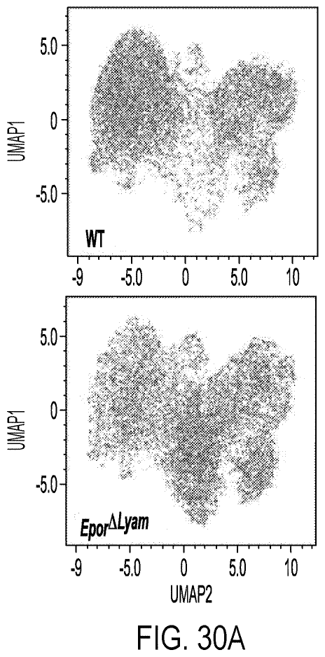

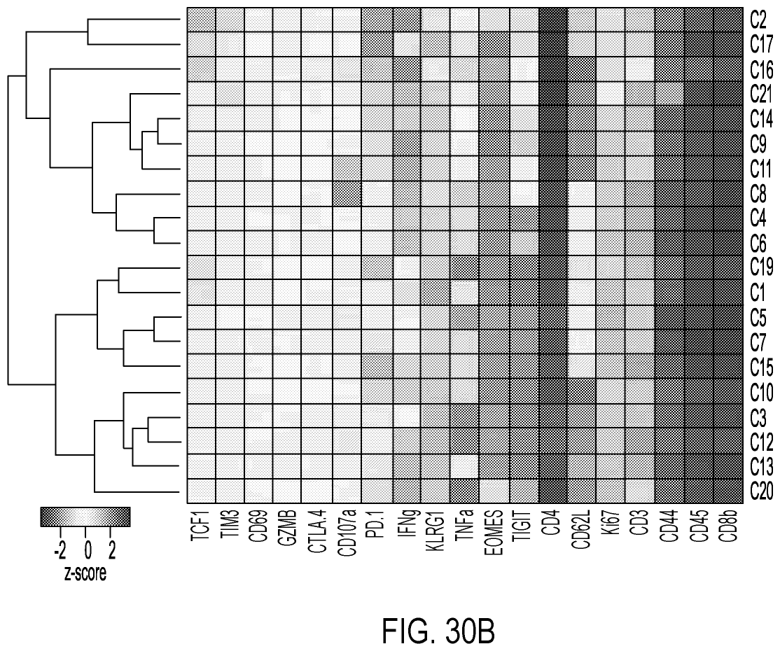

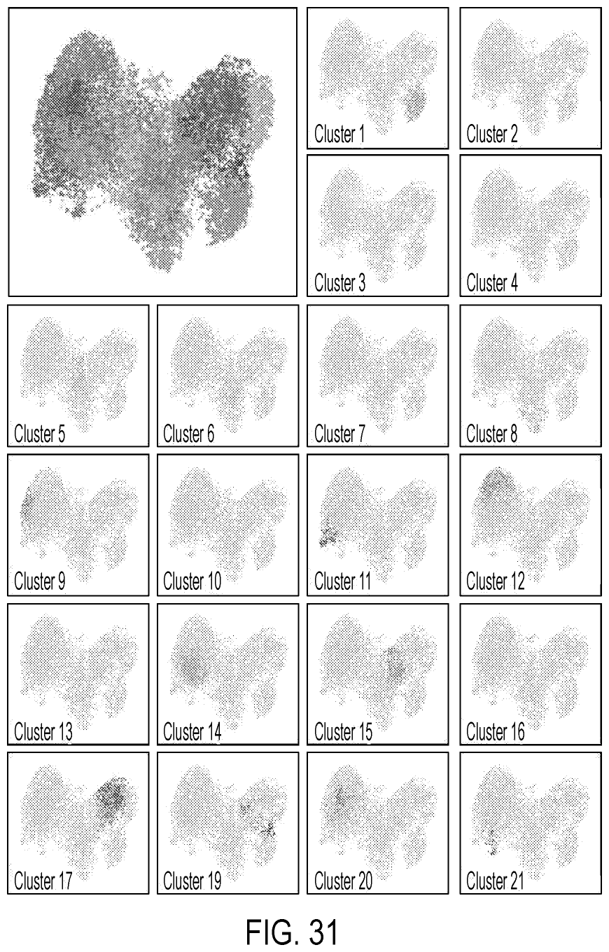

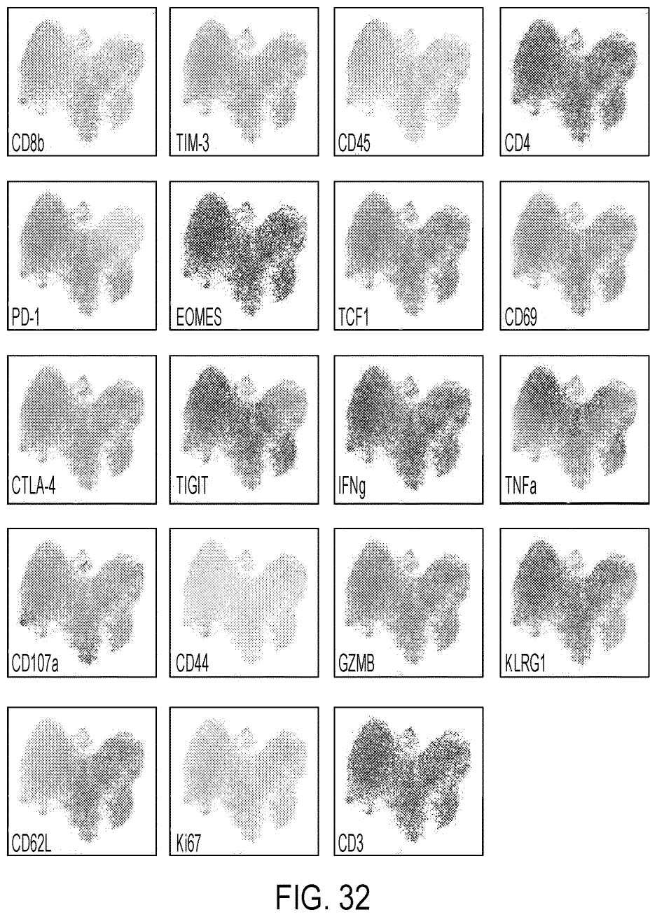

High-dimensional spectral flow cytometry was used to comprehensively characterize the tumor-infiltrating CD8⁺ T cells in both WT and Epor^{ΔLysM} mice. Trp53^{KO}/Myc^{OE} HCC tumors of WT and Epor^{ΔLysM} mice were harvested 5 weeks after HDTV. Tumor-infiltrating CD8⁺ T cells were profiled using spectral flow cytometry with 20 markers. PhenoGraph analysis of the CD8⁺ T cell population delineated 20 clusters (excluding the dead cell population), with 8 out of 20 differentially expressed markers (DEMs) contributing to the cluster definition. The antibody panel consisted of 20 markers (including viability dye) indicative of the activation, exhaustion, differentiation, memory and functional status of CD8⁺ T cells. The tumor-infiltrating CD8+T populations were analyzed with UMAP, a dimensionality reduction method, and the overlaid UMAP plot showed a dramatic subpopulation shift in Epor^{ΔLysM} mice (FIG. 11C). Twenty unsupervised clusters of CD8⁺ T cells (excluding the dead cell population) were identified with an optimized data-derived stratifying algorithm (Phenograph). Among the 20 clusters, 8 markers (CD44, CD62L, KLRG1, TNFα, IFNg, PD-1, TIGIT, EOMES) critically defined the clusters. Ablation of EPOR in macrophages led to an expansion of early activated and TNFα-producing effector memory CD8⁺ T cell subsets (clusters 4, 6, and 8) (FIG. 11D, FIG. 30, and FIG. 32), while the CD8⁺ T cells in WT mice were either terminally exhausted (clusters 2, and 17) or CD62L⁺ TNFα⁻ bystander T cells. These results suggest that EPOR acts as an immunosuppression switch in macrophages and triggering EPOR signaling stops macrophages from activating tumor-killing CD8⁺ T cells.

Two weeks after HDTV (Trp53^{KO}/Myc^{OE}-Luc⁺), C57BL/6 WT and Epor^{ΔLysM} mice were injected (IP) with 2 mg/kg of αPD-1 (RMP1-14) or IgG Isotype control every 3 days (total 5 doses). Tumor growth was monitored using luciferin-based bioluminescence imaging. Tumor growth kinetics and overall survival were measured (n=7/group). Epor^{ΔLysM} mice showed restoration of T cell immunity and expansion of PD1⁺TNFα⁺ effector memory CD8⁺ T cells, which critically control tumor growth and respond to PD1 blockade. Consistent with these data, in the Trp53^{KO} model, which does not respond to anti-PD-1 monotherapy, the combination of ablation of EPOR in macrophages and anti-PD-1 led to a profound synergistic antitumor effect, with complete tumor regression in all mice (FIG. 11E).

1.5 weeks after HDTV (Trp53^{KO}/Myc^{OE}), C57BL/6 Epor^{ΔLYsM-ERT2} mice were injected (IP) with 75 mg/kg tamoxifen or corn oil every 3 days (total 8 doses), in addition to the αPD-1 regimen. Overall survival was measured (n=7-8/group). Two weeks after HDTV (Trp53^{KO}/Myc^{OE}), C57BL/6 WT mice were injected (IP) with 20 mg/kg mEPOR-Fc or PBS weekly (total 4 doses), in addition to the αPD-1 regimen. Similar synergy was observed in established non-inflamed tumors treated with tamoxifen-induced EPOR deletion in macrophages (FIG. 11F) or pharmacological inhibition of EPO/EPOR signaling using a recombinant mouse EPOR-Fc chimera protein (mEPOR-Fc) as a decoy receptor to neutralize EPO (FIG. 11G). In combination, these data indicate EPO/EPOR signaling in macrophages suppresses T cell activation and plays a central role in generating a non-inflamed TME, and that targeting this pathway in macrophages can transform non-inflamed HCC into T cell-inflamed and ICB-responsive HCC.

### Example 16—EPO Transforms Macrophages into Immunoregulatory Cells Via EPOR

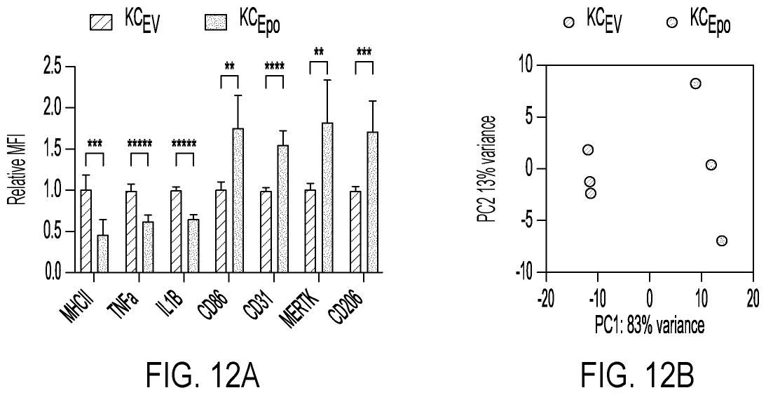

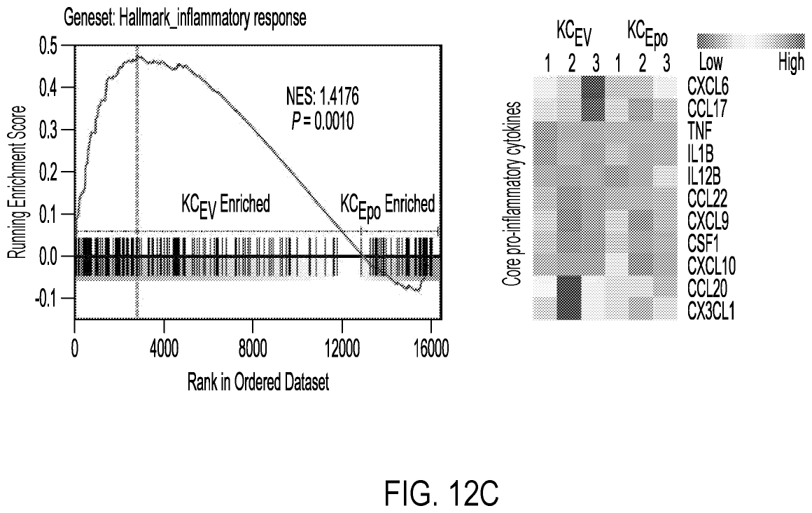

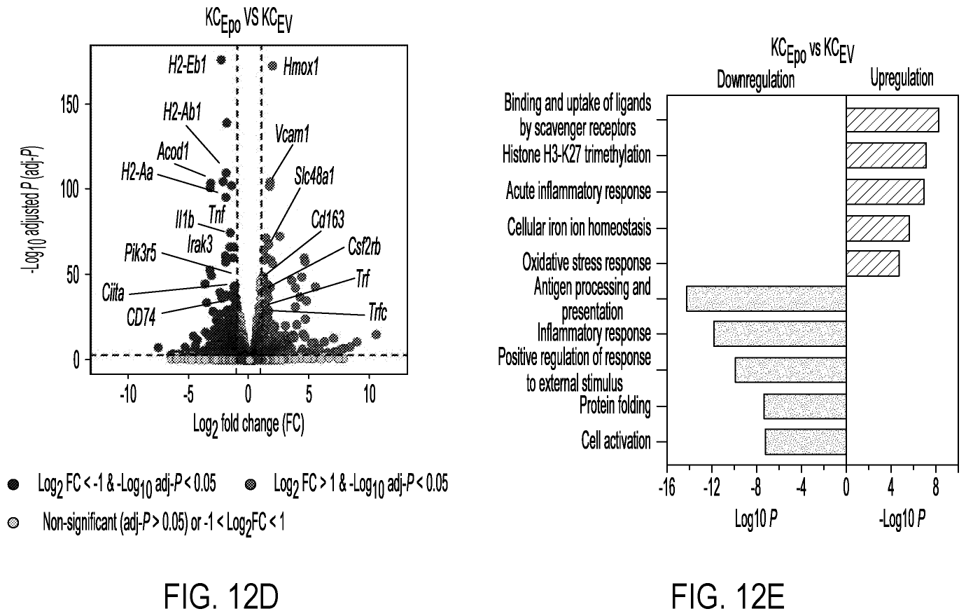

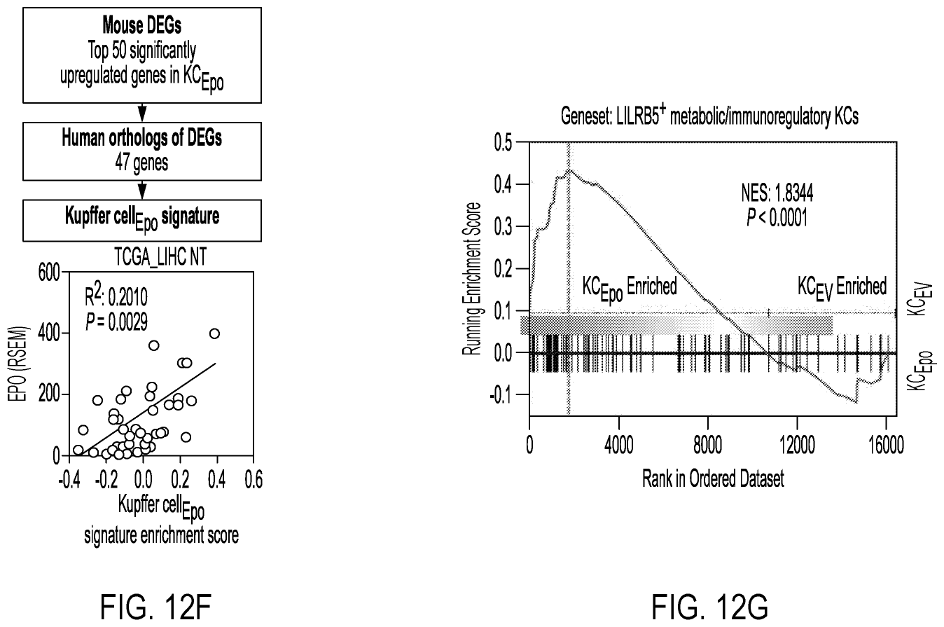

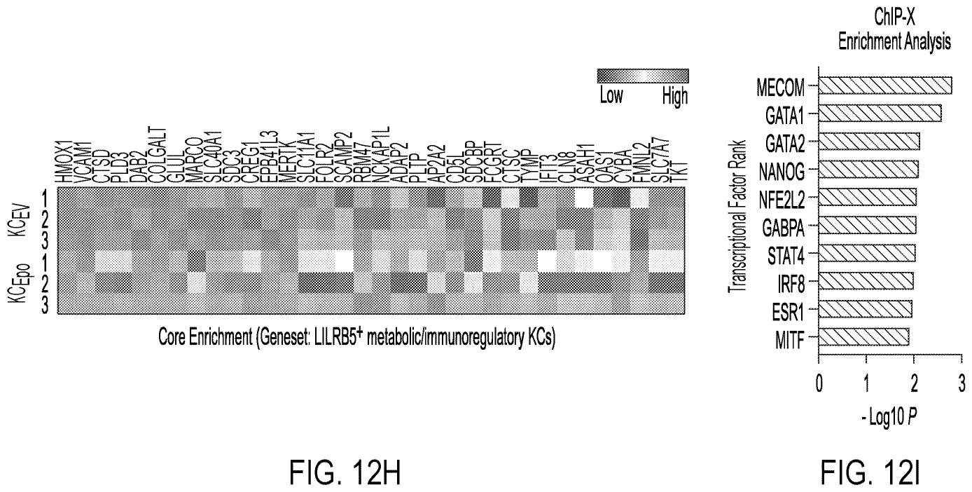

EPO programs macrophages via EPOR to promote immunosuppression and hinder antitumor T cell immunity. KCs were isolated by FACS from adjacent liver tissues of Hepa1-6_EV (KC_{Ev}) and Hepa1-6_Epo^{OE} (KCEpo) tumors at day 18 post-implantation. KCEv and KC_{Epo} were analyzed with flow cytometry and subjected to bulk-RNAseq. In both mice and humans, macrophages undergo differentiation into KCs or adopt KC-like characteristics in response to EPO. Furthermore, in HCC-bearing mice, EPO stimulation leads to a reduction in the expression of inflammatory and antigen-presenting proteins such as MHCII, TNFα, and IL-1β in KCs, accompanied by an increase in regulatory proteins including CD31, MERTK, and CD206 (FIG. 12A).

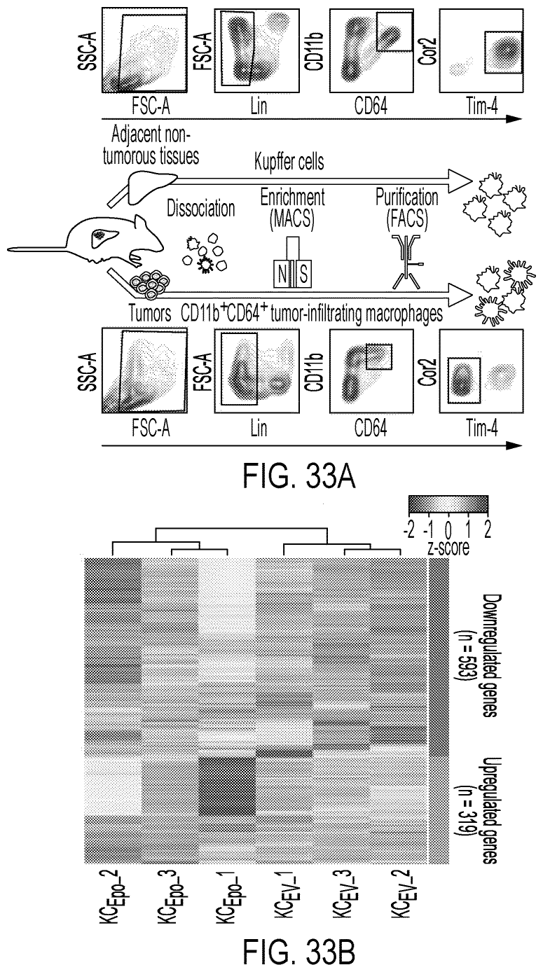

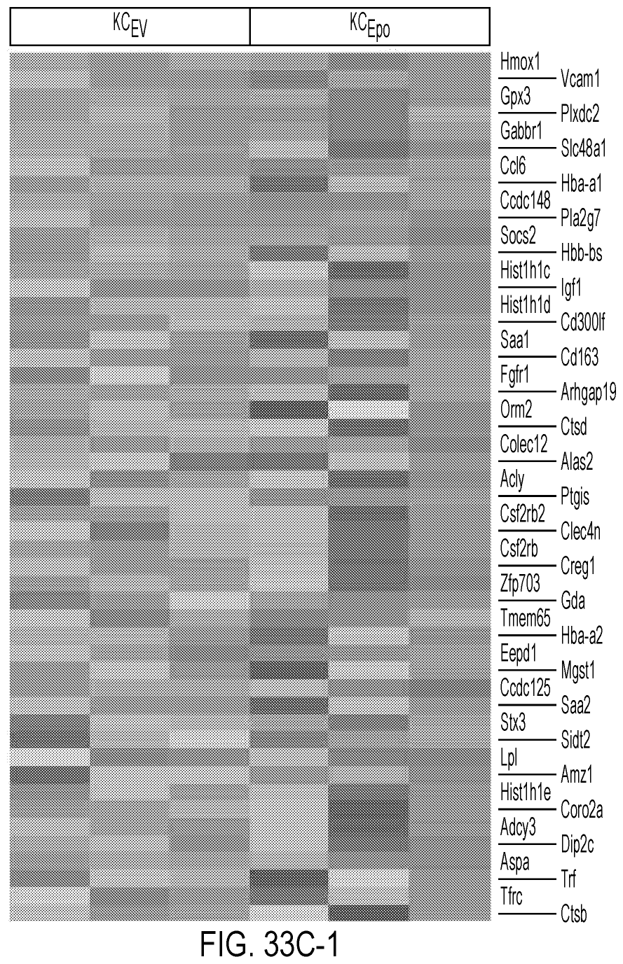

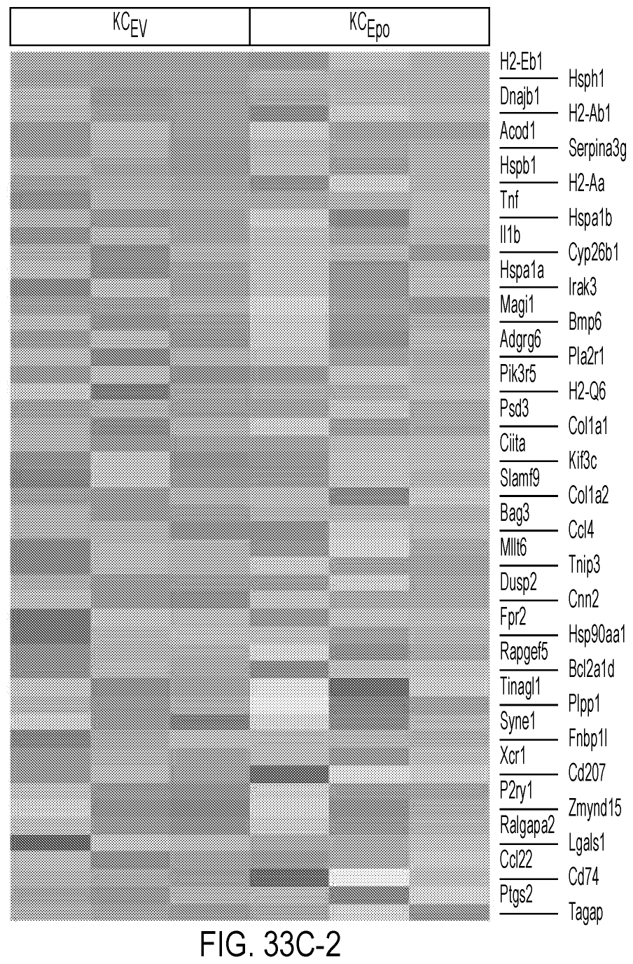

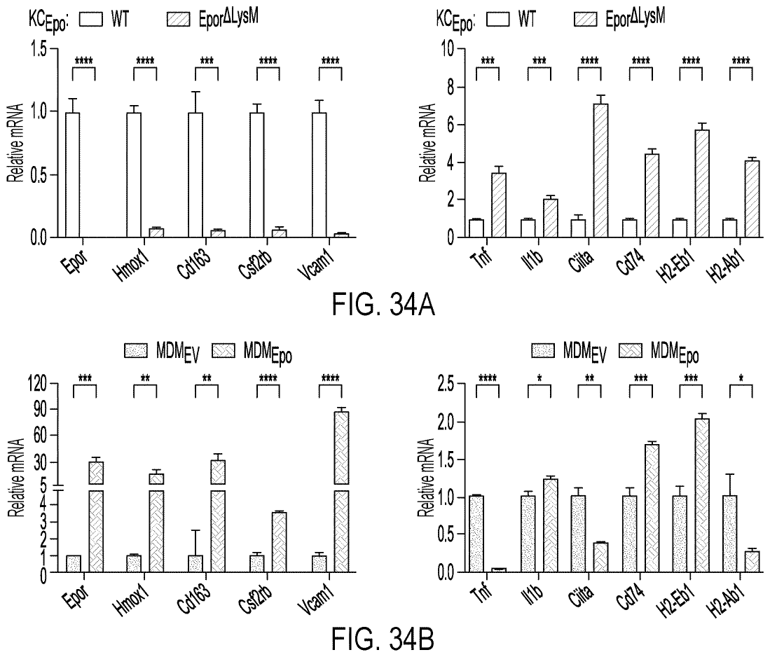

TIM-4⁺ macrophages (KCs) from mice bearing Hepa1-6_EV (KCEV) and Hepa1-6_Epo^{OB} (KCEpo) were isolated and subjected to bulk-RNA sequencing. Principal component analysis of these data showed that EPO/EPOR signaling induced a distinct transcriptomic profile in KCs associated with downregulation of inflammatory responses and secretion of cytokines important for T cell activation and recruitment, including Il1b and Cxc110 (FIGS. 12B-12C). Among the differentially expressed genes, 593 upregulated and 319 downregulated genes were identified in KC_{Epo} compared to KCEV (Log 2 fold change>1.0, P-adjusted value<0.05; FIG. 12D and FIGS. 33A-33B). Pathway enrichment analysis revealed that the top 50 upregulated genes in KC_{Epo} were involved in scavenger receptor-mediated ligand binding and uptake, histone trimethylation, resolving acute inflammation, and cellular iron metabolism, while the top 50 downregulated genes in KC_{Epo} were involved in antigen presentation, inflammation, response to external stimuli, protein folding, and cell activation (FIG. 12E and FIG. 33C). Importantly, most of these changes induced by EPO in KCs were also observed in MDMs and were abolished by knockout of EPOR in macrophages (FIG. 34), confirming that the gene regulation is conserved in different populations of macrophages in HCC, and occurs via the EPO/EPOR signaling axis.

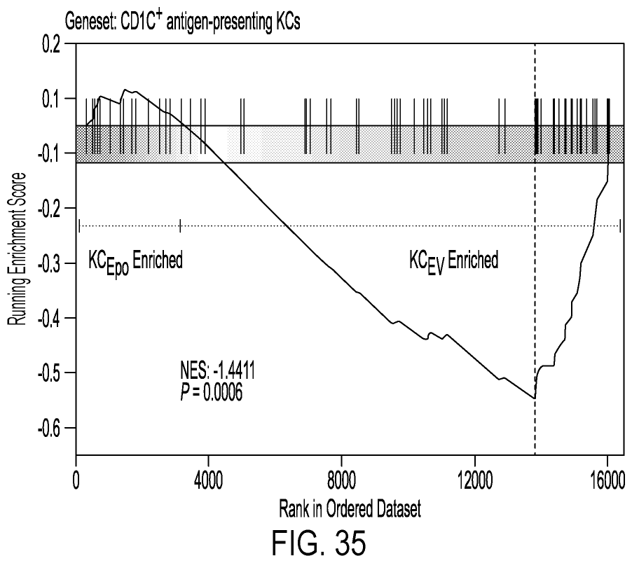

To determine if the macrophages in human HCC exhibit similar regulation by EPO, the top 50 significantly upregulated genes in KC_{Epo} as the KC_{Epo} signature were defined. Analysis of human HCC samples in the TCGA database revealed a positive correlation between the expression level of EPO and this KC_{Epo} signature (FIG. 12F). Further analysis showed that KC_{EV} resembled human CDIC⁺ antigen-presenting KCs, while KC_{Epo} resembled human ILIRB5⁺ metabolic/immunoregulatory KCs (FIG. 12G and FIG. 35). The expression profile of EPOR⁺ liver macrophages strongly aligned with ILIRB5⁺ KCs. Collectively, these results indicate that the EPO/EPOR axis regulates the functions of liver macrophages in human HCC and promotes the expansion of the immunoregulatory KC subset in HCC, as it does in mice.

### Example 17—NRF2 is a Critical Downstream Mediator of the EPO/EPOR Axis in Macrophages

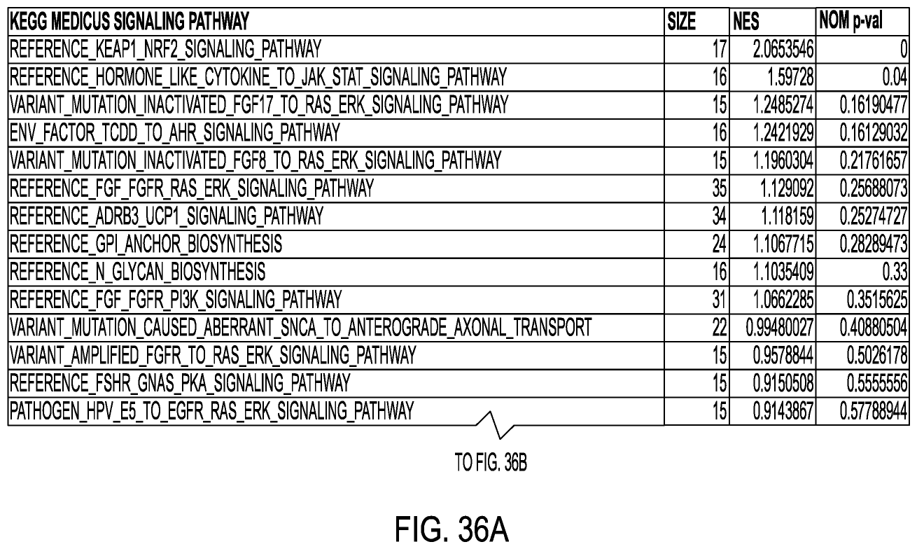

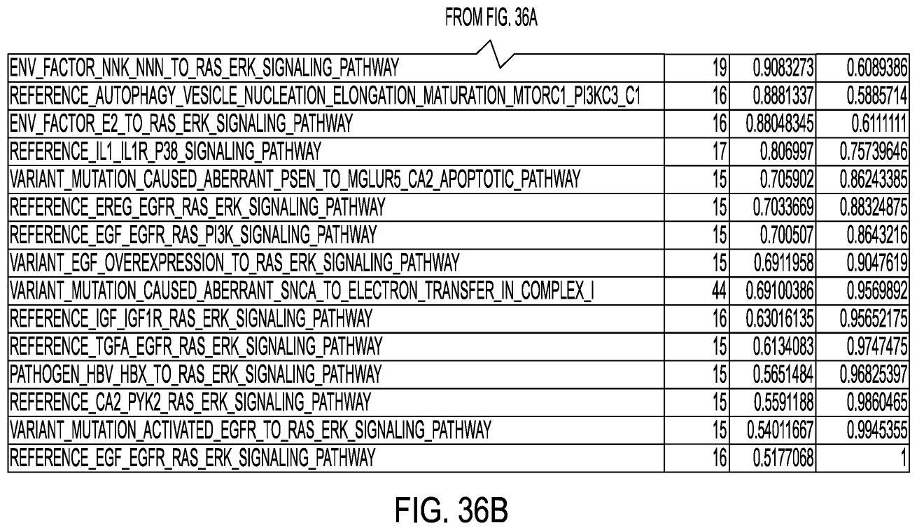

Thirty-four genes critically enriched in KC_{Epo} for human ILIRB5⁺ KCs were identified using geneset enrichment analysis (GSEA) (FIG. 12H). Subsequent ChIP-X enrichment analysis showed that general transcription factors governing hematopoiesis and stem cell biology, such as MECOM, GATA1, GATA2, and NANOG, can regulate these genes (FIG. 12I). Notably, many of these genes, particularly those involved in iron metabolism and antioxidation, are also under the control of the transcriptional factor nuclear factor erythroid 2-related factor 2 (NFE2L2 or NRF2) (FIG. 12I). Furthermore, pathway analysis utilizing the KEGG database revealed the KEAP1-NRF2 signaling pathway as the most enriched and significant in EPO-stimulated KCs (FIG. 36).

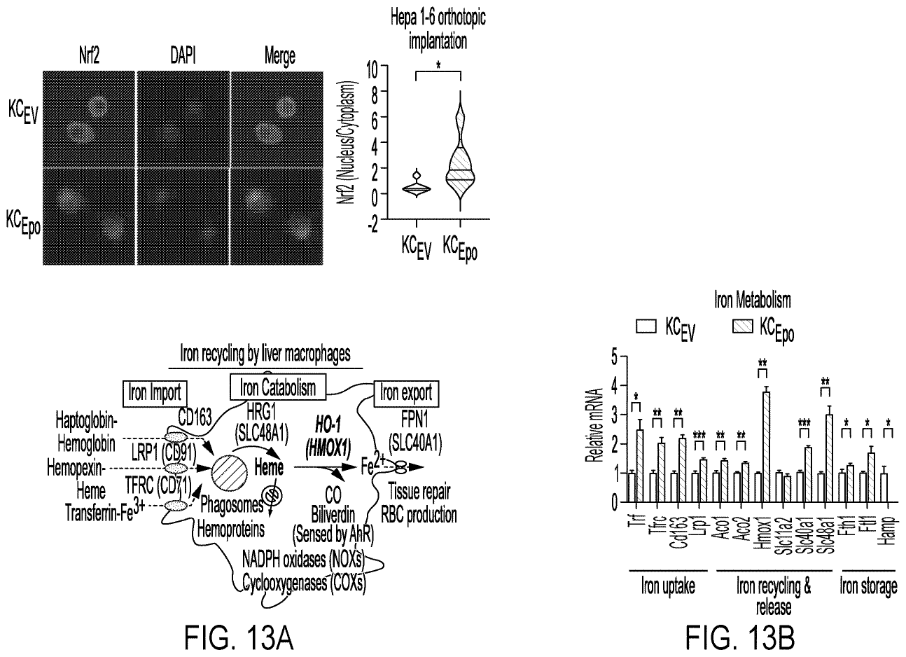

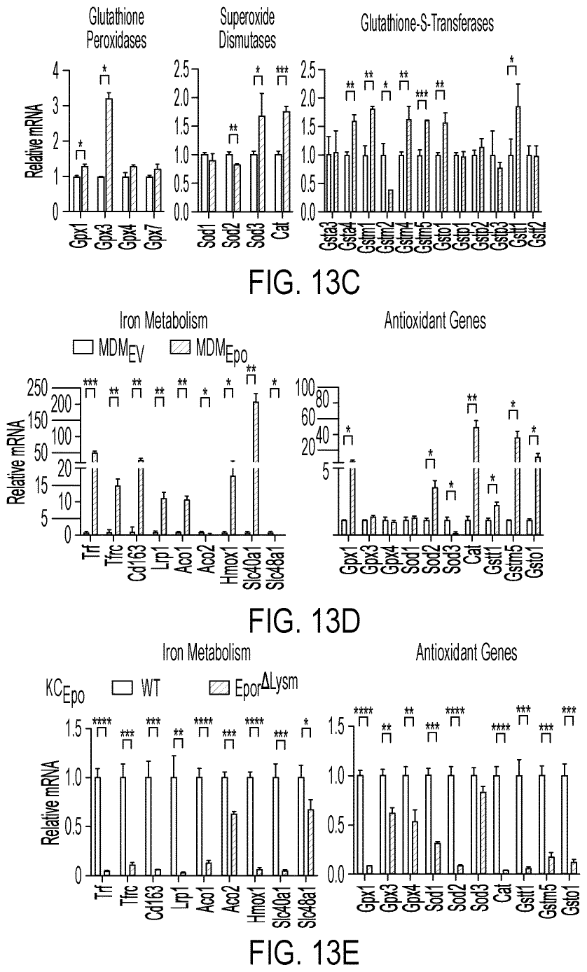

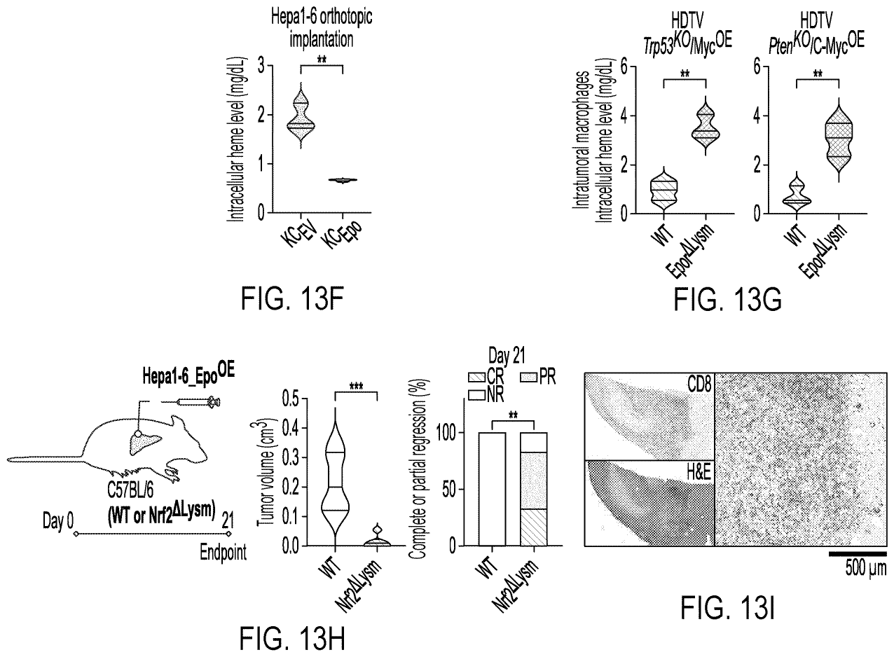

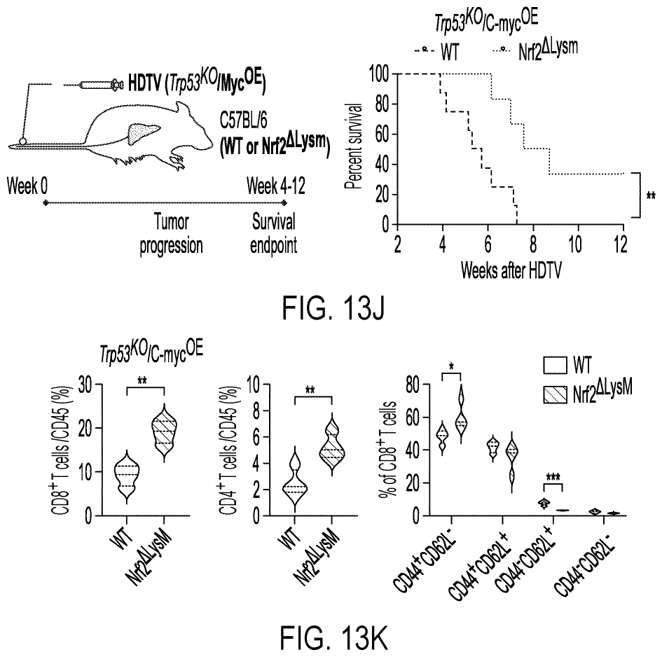

EPO stimulation induced NRF2 nuclear localization in KCs of HCC-bearing mice (FIG. 13A), suggesting a role for EPO in modulating NRF2 signaling within macrophages and implicating iron metabolism and antioxidation pathways in EPO-induced immune regulation. Heme oxygenase-1 (HMOX1) is the most significantly upregulated gene in EPO-stimulated KCs. HMOX1 is an enzyme that catalyzes the degradation of heme, a component of hemoglobin, into iron, carbon monoxide and biliverdin. Heme is essential for macrophages to generate pro-inflammatory cytokines and reactive oxygen species, but its depletion shifts macrophages to anti-inflammatory phenotypes. Along with HMOX1, EPO/EPOR activation also upregulated several genes involved in iron recycling and antioxidant production in KCs and MDMs (FIGS. 13B-13D). Most of these genes are known targets of NRF2 regulation, and the absence of EPO/EPOR signaling results in a significant reduction in their expression levels (FIG. 13E).

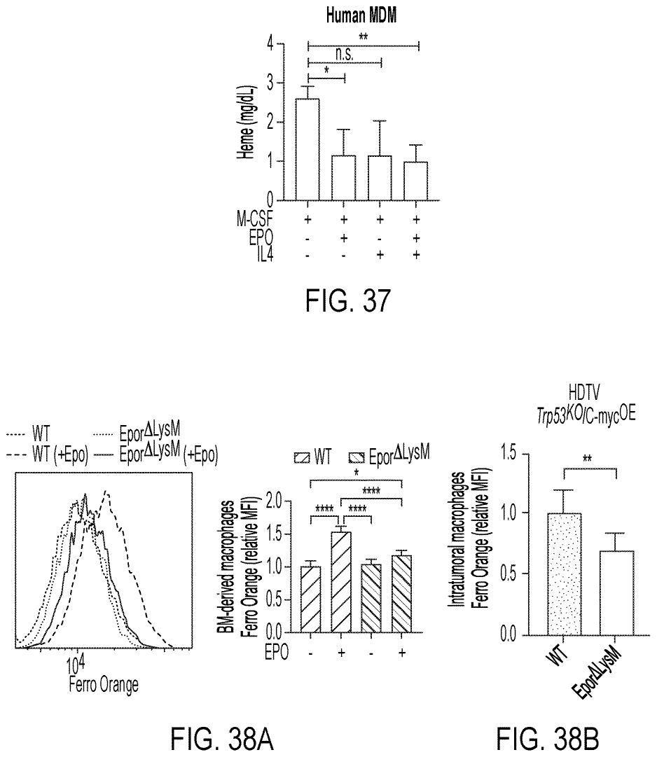

EPO stimulation lowered the heme levels in KCs in the Hepa1-6 orthotopic model, as well as human monocyte-derived macrophages (FIG. 13F and FIG. 37). Moreover, EPOR deletion increased the intracellular heme levels in intratumoral macrophages in Trp53^{KO} and Pten^{KO} non-inflamed HCC models (FIG. 13G). Additionally, EPO stimulation temporarily elevated intracellular free iron levels, likely due to heme breakdown, an effect reversed by EPOR deletion (FIG. 38). To further demonstrate the role of NRF2 in EPO-mediated macrophage modulation, LysM-driven Nrf2^{-/-} mice were generated, which lack Nrf2 expression in macrophages (Nrf2^{ΔLysM}). Hepa1-6_Epo^{OE} tumors could not escape T cell immunosurveillance (FIGS. 13H-131), and in Nrf2^{ΔLysM} mice, non-inflamed HCC progressed much more slowly and was associated with an increase in CD8⁺ TEM infiltration (FIGS. 13J-13K). In combination, these data reveal that EPO/EPOR signaling modulates macrophage polarization and immunosuppression in HCC by regulating heme metabolism and NRF2 activation.

### Materials and Methods (for Examples 11-17)

### Mice

LysM^{Cre/Cre} mice were crossbred with Nrf2fl/fl and Epor^{fl/fl} mice to generate LysM^{Cre}; Nrf2^{fl/fl} (Nrf2^{ΔLysM}) mice and LysM^{Cre}; Epor^{fl/fl} (Epor^{ΔLysM}) mice, respectively. Ms4a3^{Cre/Cre} mice were crossbred with ROSA26-EFGP to generate Ms4a3^{Cre/Cre}; ROSA26-EFGP (Ms4a3^{EGFP}) mice. LysM-ERT2 mice were crossbred with Epor^{fl/fl} mice to generate LysM-Cre; Epor^{f1/fl} (Epor^{ΔLysM-ERT2}) mice. Orthotopic implantation studies were performed in mice between 5 to 7 weeks of age, while HDTV studies were performed between 8 to 10 weeks of age.

### Cell Lines

The Hepa1-6 murine HCC line was acquired from ATCC (CRL-1830) and the 293 FT line was acquired from Thermo Fisher Scientific. Cells were grown in Dulbecco's Modified Eagle Medium (DMEM) supplemented with 4 mM L-glutamine, 10% Fetal Bovine Serum (FBS), and 1% Penicillin Streptomycin. Cells were routinely tested for mycoplasma by PCR and all tests were negative.

### Generation of Epo-Overexpressing Hepa1-6 Cells

Epo-ORF (open reading frame) was inserted into lentiviral vector pLVX-EF1a-IRES-Puro by using EcoRI and BamHI restriction sites. Lentiviral particles were packaged in 293 FT cells using the pCMV-VSVG and psPAX2 envelope and packaging plasmids. HEK-293T cells were transfected with Lipofectamine 2000 (Thermo Fisher Scientific) in DMEM. Viral supernatant was collected after 24 hours and filtered through a 0.45 μm filter and added to Hepa1-6 cells in the presence of 8 μg/ml polybrene (Sigma Aldrich). Transduced Hepa1-6 cells were selected in puromycin (Invivogen) at a concentration of 1 μg/ml. Conditioned media were collected from selected cells, and Epo-overexpression efficiency was confirmed by ELISA.

### Tumor Models

For orthotopic implantation, 3×10⁶ Hepa1-6 cells were resuspended in 15 μL of Matrigel Basement Membrane Matrix (Corning) and injected into the left lobes of the livers of the mice. For hydrodynamic tail vein injection (HDTV) model, sterile saline/plasmid mix, with a total volume corresponding to 10% of body weight, was injected into the lateral tail vein over 8 to 10 seconds. The sterile saline/plasmid mix contained a total of 20 μg of pX330-sgTrp53/Pten/Keap1 or pX333-sgTrp53-sgEpo, 10 μg of pT3-EF1a-Myc or pT3-EF1a-Myc-IRES-Luciferase, and 2 μg of pCMV-SB13. sgRNAs were inserted into pX330 and pX333 by using BbsI and BsaI restriction sites. The sequences of the sgRNAs used in this study are provided in Table 2. Plasmids for HDTV were prepared using PureLink Endotoxin-Free Maxi Plasmid Purification Kit (Thermo Fisher Scientific). To monitor the growth of luciferase-expressing tumors, mice were administered 100 mg/kg D-luciferin (Gold Biotechnology) through intraperitoneal injection prior to bioluminescence imaging using an IVIS Spectrum imager (Caliper Life Sciences). At endpoints, tumors were measured with a caliper and calculated with the formula length×width×depth×0.52 (cubic millimeters). Tumors and adjacent non-tumorous liver tissues were harvested for histology analysis or dissociated into single cell suspension for downstream analysis.

Preparation of Single Cell Suspension from Tissues

Tissues (including human/mouse normal livers, HCC tissues and adjacent non-tumorous tissues) were collected in ice-cold PBS. All human tissues underwent processing within 24 hours following collection, whereas mouse tissues were processed promptly after harvesting. Tissues were subjected to mincing, digestion with Liberase™ (Roche) and DnaseI (Sigma Aldrich) RPMI-1640, and dissociation with gentleMACS Dissociator (Miltenyi Biotec) for 20 minutes at 37° C. Dissociated samples were filtered through a 70 μm cell strainer and ACK lysis buffer was used to lyse the red blood cells. Cells were resuspended in PBS supplemented with 2% FBS and 2 mM EDTA (FACS Buffer, FB) for fluorescence-activated cell sorting (FACS), magnetic cell separation or flow cytometry analysis.

### Flow Cytometry Analysis and Cell Sorting

Cells were stained for viability using LIVE/DEAD Fixable Blue Dead Cell Stain (Thermo Fisher Scientific) on ice for 30 minutes. Cells were treated with Fc Block (BD Biosciences, 2.4G2) at room temperature for 10 minutes. Surface staining was performed for 30 minutes at 4° C. prior to fixation. For intracellular staining, cells were fixed and permeabilized using the eBioscience FoxP3 Fixation/Permeabilization kit (Thermo Fisher Scientific). Intracellular staining was performed at room temperature, and cells were washed twice in permeabilization buffer prior to transfer back into FB. The following anti-mouse antibodies were used for flow cytometric analysis or FACS: CD45 (BioLegend, 30-F11), CD11b (BioLegend, M1/70), Ly-6C (BioLegend, HK1.4), Ly-6G (BioLegend, 1A8), CD11c (BioLegend, N418), CD86 (BD Biosciences, GL1), CD8a (BD Biosciences, 53-6.7), CD40 (BD Biosciences, 3/23), F4/80 (BioLegend, BM8), CX3CR1 (BioLegend, SA011F11), I-A/I-E (BioLegend, M5/114.15.2), CCR2 (BioLegend, SA203G11), CD103 (BioLegend, 2E7), CD3 (BioLegend, 17A2), FoxP3 (eBioscience, FJK-16s), CD25 (eBioscience, eBio7D4), CD44 (BioLegend, IM7), CD62L (BioLegend, MEL-14), CD4 (BioLegend, RM4-5), CD8b (BioLegend, YTS156.7.7), CD69 (BioLegend, H1.2F3), PD-1 (BD Biosciences, 29F.1A12), NK1.1 (BioLegend, PK136), KLRG1 (BioLegend, 2F1/KLRG1), CD64 (BD Biosciences, X54-5/7.1), CD19 (BioLegend, 1D3/CD19), Granzyme B (BioLegend, GB11), CTLA-4 (BioLegend, UC10-4B9), TIGIT (BD Biosciences, 1G9), TIM-3 (BioLegend, RMT3-23), TCF1/7 (BD Biosciences, S33-966), EOMES (BD Biosciences, X4-83), CD107a (BD Biosciences, 1D4B), IFNg (BioLegend, XMG1.2), Ki67 (BioLegend, 16A8), TNFa (BD Biosciences, MP6-XT22), TIM-4 (BD Biosciences, RMT4-54), CD71 (BioLegend, RI7217), Ter-119 (BioLegend, TER-119), CLEC4F (BioLegend, 3E3F9) VSIG4 (Invitrogen, NLA14), Gr-1 (BioLegend, RB6-8C5), EPOR (ImmunEdge, in-house), Helios (BioLegend, 22F6). The following anti-human antibodies were used for flow cytometric analysis or FACS: CD45 (BioLegend, HI30), CD68 (BioLegend, Y1/82A), CD14 (BioLegend, M5F2), CD163 (BioLegend, GHI/61), CD131 (BioLegend, 3D7), HLA-DR (BioLegend, L243), CD11b (BioLegend, LM2), EPOR (R&D Systems, 38409), CD4 (BioLegend, SK3), CD3 (BioLegend, SK7), CD8 (BioLegend, SK1), CD19 (BioLegend, HIB19), CD56 (BioLegend, QA17A16). For traditional flow cytometry analysis, samples were run on LSRFortessa cytometer (Becton Dickinson) and analyzed using FlowJo V10 software (TreeStar). Absolute counts were determined by using SPHERO AccuCount Fluorescent Particles (Spherotech), according to the manufacturer's guidelines. For spectral or high-dimensional flow cytometry analysis, samples were run on Cytek Aurora and analyzed using FlowJo V10 software. The FlowJo plugin Downsample was used to select 2500 gated live CD3+CD8⁺ T cells from each sample. All samples were concatenated prior to the downstream analysis. The FlowJo plugin uniform manifold approximation and projection (UMAP) was used for dimensionality reduction to visualize Flowjo high-parameter data sets in 2-dimensional space, and the plugin PhenoGraph was used to group data into different unsupervised clusters. Heatmap was generated using the heatmap2 and flowCore packages in R. For FACS, samples were run on FACSAria II (Becton Dickinson). Mouse Kupffer cells (KCs) and monocyte-derived macrophages were pre-enriched by magnetic cell separation against F4/80 (Miltenyi Biotec) prior to FACS.

### RNA Extraction and NGS Library Preparation and RT-qPCR

RNA was extracted using RNEasy Plus mini kits (Qiagen). Library preparation and sequencing were performed by MedGenome, Inc. (Foster City, CA). For mouse KCs in FIG. 11, libraries were prepared using the Illumina TruSeq stranded mRNA kit. For human EPOR⁺ and EPOR macrophages, libraries were prepared using the Takara SMART-Seq v4 Ultra low Input RNA kit. Libraries were checked for size using the Agilent DNA 100 kit on a BioAnalyzer (Agilent) and quantified by qPCR using the KAPA Library Quantification Kit for Illumina. Libraries were sequenced on NovaSeq 6000 Flow Cells (S4) at 2×100 cycles paired-end to a depth of approximately 20M paired reads per sample. To validate some bulk-RNAseq results, RNA was converted to cDNA with the iScript cDNA synthesis kit (BioRad). qRT-PCR amplification was performed using PowerUp SYBR Green Master Mix (Thermo Fisher Scientific) with specific primers. The sequences of the primers used in this study are provided in Table 3.

### RNA Sequencing Analysis

Raw sequencing reads were filtered, adapters trimmed using fastq-mcf program (v1.05), and data quality check was performed using FastQC (v0.11.8). Contamination removal was performed using Bowtie2 (v2.5.1) (B. Langmead, S. L. Salzberg, Fast gapped-read alignment with Bowtie 2. Nat Methods 9, 357-359 (2012)). The paired-end reads were aligned to the reference Human or Mouse genome downloaded from the Ensembl database, and the alignment was performed using STAR (2.7.3a)(A. Dobin et al., STAR: ultrafast universal RNA-seq aligner. Bioinformatics 29, 15-21 (2013)). The raw read counts were estimated using HTSeq (v0.11.2) (S. Anders, P. T. Pyl, W. Huber, HTSeq-a Python framework to work with high-throughput sequencing data. Bioinformatics 31, 166-169 (2015)). Additionally, the aligned reads were used for estimating expression of the genes using cufflinks (v2.2.1). The expression values were reported in FPKM (Fragments per kilobase per million) units for each gene. Differential gene expression analysis was performed using DESeq2 (M. I. Love, W. Huber, S. Anders, Moderated estimation of fold change and dispersion for RNA-seq data with DESeq2. Genome Biol 15, 550 (2014)). Counts were transformed and normalized using a regularized log transformation. Principal component analysis (PCA) was performed using the R statistics package and the PCA plots were calculated using all genes differentially expressed between the samples. Volcano plots were generated using ggplot2. Differentially expressed genes in blue were defined as genes with an adjusted p-value<0.05 and |fold change|>2. Heatmaps were generated using the heatmap2 package, which clusters based upon Euclidean distances. Gene set enrichment analysis (GSEA) was performed using GSEA v4.3 software (A. Subramanian et al., Gene set enrichment analysis: a knowledge-based approach for interpreting genome-wide expression profiles. Proc Natl Acad Sci USA 102, 15545-15550 (2005); V. K. Mootha et al., PGC-1 alpha-responsive genes involved in oxidative phosphorylation are coordinately downregulated in human diabetes. Nat Genet 34, 267-273 (2003)). Pathway enrichment analysis and cell type analysis were performed using Metascape.

### Immunohistochemical (IHC) Analysis

Prior to paraffin embedding, tissues were fixed in 10% buffered formalin and washed with 75% ethanol. Heated paraffin sections were dewaxed in xylene followed by ethanol gradation. Antigens were retrieved in ImM EDTA buffer (pH 7.8) by boiling for 15 minutes. Following blocking endogenous enzymes with 3% hydrogen peroxide and preventing non-specific binding with 2% bovine serum albumin (BSA), slides were stained with primary antibodies overnight at 4° C. For horseradish peroxidase (HRP) immunodetection, slides were stained with En Vision+System-HRP Labelled Polymer (Agilent) and developed with 3,3′-Diaminobenzidine tetrahydrochloride (Sigma-Aldrich). Slides were counterstained with hematoxylin. For immunofluorescent (IF) detection, primary antibodies were conjugated with fluorescent dyes using Zenon Antibody Labeling Kits (Thermo Fisher Scientific) or DyLight Conjugation Kits (Abcam) prior to staining. Slides were counterstained with 4′,6-diamidino-2-phenylindole (DAPI). Slides were imaged on a Keyence BZ-X810 microscope. The following antibodies were used for IHC: anti-mouse CD8a (Cell Signaling, D4W2Z), anti-mouse FoxP3 (Cell Signaling, D608R), anti-human CD68 (Cell Signaling, E307V), anti-human EPOR (ImmunEdge, in-house).

Preparation of siRNA-Loaded Liposomes

FITC-labelled siNTC (sense: 5′-CGUUAAUCGCGUAUAAUACGCGUdAdT-3′ (SEQ ID NO: 5993); antisense: 5′-ACGCGUAUUAUACGCGAUUAACGdAdT-3′-FITC (SEQ ID NO: 5994)) and siEpor (sense: 5′-CCCAGAGAGCGAGUUUGAGGGUCdTdC-3′ (SEQ ID NO: 5995); antisense: 5′-GACCCUCAAACUCGCU CUCUGGGdTdC-3′-FITC (SEQ ID NO: 5996)) were synthesized by Dharmacon (Horizon Discovery). siRNA was dissolved in 10 mM sodium citrate (0.45 mg/mL). 1,2-Distearoyl-sn-glycero-3-phosphocholine (DSPC), 1,2-dipalmitoyl-sn-glycero-3-phosphocholine (DPPC), cholesterol and DSPE-PEG-NH2 (MW: 3400) at a weight ratio of 1:1:0.5:0.4 were then dissolved in chloroform and the lipid mixture was evaporated to produce dry liposome powder. Dry liposome powder and siRNA (1:40 w/w) were resuspended in UltraPure DNase/RNase-Free Distilled Water (Thermo Fisher Scientific) and sonicated to form siRNA-loaded liposomes of desired molecular size. The molecular size and siRNA encapsulation efficiency of liposomes were determined as previously described (X. Huang et al., Synthesis of siRNA nanoparticles to silence plaque-destabilizing gene in atherosclerotic lesional macrophages. Nat Protoc 17, 748-780 (2022)).

### Measurement of EPO, Heme and Intracellular Free Iron

To assess plasma EPO levels in mice with HCC, blood was obtained via retro-orbital bleeding before sacrifice. The collected blood was centrifuged at 1500×g for 15 minutes at 4° C. to isolate the plasma layer. To verify the overexpression efficacy of EPO in Hepa1-6_Epo^{OE} cells, conditioned media from both Hepa1-6_EV and Hepa1-6_Epo^{OE} cells were gathered. Plasma EPO levels were determined using the Mouse Erythropoietin/EPO Quantikine ELISA Kit (R&D Systems). To assess the intracellular heme levels in macrophages, mouse Kupffer cells (KCs) and monocyte-derived macrophages were purified with magnetic cell separation against F4/80 (Miltenyi Biotec) and FACS. The purified macrophages were lysed, and intracellular heme levels were determined using a Heme Assay Kit (Sigma-Aldrich). All colorimetric test results were recorded using a Victor X4 Multilabel Reader (Perkin Elmer). Intracellular free iron levels of tumor macrophages and bone-marrow-derived macrophages were determined using FerroOrange (Cell Signaling Technology) with flow cytometry analysis.

### NRF2 Subcellular Localization

Mouse KCs were purified using magnetic cell separation followed by FACS. Purified KCs were concentrated onto microscope slides using StatSpin Cytofuge 2 cytocentrifuge (Beckman Coulter). Slides were fixed with 4% paraformaldehyde (PFA) and permeabilized with PBS containing 0.2% Triton X-100. Slides were blocked with 1% BSA, 22.52 mg/mL glycine in PBST (PBS+ 0.1% Tween 20). Rabbit anti-human/mouse NRF2 antibody was conjugated to FITC using Zenon Antibody Labeling Kit (Thermo Fisher Scientific). Slides were stained with anti-NRF2 antibody overnight at 4° C. and counterstained with DAPI. Slides were imaged on a Zeiss LSM 700 confocal microscope. The subcellular localization of NRF2 in the cell nuclei or cytoplasm was defined on the basis of colocalization with DAPI. The NRF2 signal was quantified using the colocalization tool in ZEN Blue software (Zeiss).

### Immune Cell Depletion

C57BL/6 wild-type mice were orthotopically implanted in the liver with 3×10⁶ Hepa1-6_Epo^{OE} cells. On Days 14, 17, and 20 after implantation, 2 mg/kg ctCD25 (BioXCell, PC-61.5.3), ctCTLA-4 (BioXCell, 9H10), ctCCR8 (BioLegend, SA214G2) or IgG control (BioXCell, hamster polyclonal) was injected intraperitoneally to deplete Tregs. Tumors and spleens were collected on Day 21 for further analysis. For CD4⁺ and CD8⁺ T cell depletion studies, 2 weeks after HDTV, mice received intraperitoneal injections of 4 mg/kg of ctCD8 (BioXCell, YTS169.4), ctCD4 (BioXCell, GK1.5) or IgG control (BioXCell, LTF-2) twice for the first week and then once weekly (total 6 doses). Overall survival was measured. For EDMC depletion studies, 2 weeks after HDTV, mice were given IP injections of 2 mg/kg of ctTer-119 (TER-119) or IgG Isotype control every 3 days (total 9 doses). Overall survival was measured.

### PD-1 Blockade

Following administration of plasmid cocktails via HDTV, mice received intraperitoneal injections of 2 mg/kg of ctPD-1 (BioXCell, RMP1-14) or IgG Isotype control (BioXCell, 2A3) every 3 days (total 5 doses). Tumor growth kinetics were assessed using luciferin-based bioluminescence imaging, and overall survival was determined.

### EPO Administration

To examine the effects of EPO on Keap1^{KO} tumor progression, C57BL/6 wild-type mice were injected with plasmid cocktail (20 μg of pX330-sgKeap1, 10 μg of pT3-EF1a-Myc-IRES-Luciferase, and 2 μg of pCMV-SB13) via HDTV. 2 weeks after HDTV, mice received intraperitoneal injections of either PBS or 50 IU of rHuEPO (PeproTech) daily for 3 weeks, and tumor growth kinetics were monitored using luciferin-based bioluminescence imaging.

### Induction of EPOR Ablation in Macrophages by Tamoxifen

Tamoxifen (Sigma-Aldrich) was dissolved in corn oil at a concentration of 20 mg/ml by shaking overnight at 37° C. 1.5 weeks after HDTV C57BL/6 Epor^{ΔLysM-ERT2} mice were injected IP with 75 mg/kg tamoxifen or corn oil every 3 days (total 8 doses).

### In Vitro Studies of Human Macrophages

Fresh human PBMCs were isolated from whole blood using SepMate PBMC Isolation Tubes (StemCell Technologies). PBMCs were cultured in ImmunoCultTM-SF Macrophage Differentiation Medium supplemented with 50 ng/ml of human recombinant M-CSF (PeproTech). On Day 6, macrophages were treated with 10 ng/ml of human recombinant IL-4 (PeproTech) and 100 ng/ml of rHuEPO. On Day 8, macrophages were harvested, and the intracellular heme level was measured.\

### In Vitro Studies of Mouse Macrophages

Fresh mouse bone marrow cells (BMCs) were isolated from tibia and femur. BMCs were cultured in complete RPMI Medium supplemented with 50 ng/ml of mouse recombinant M-CSF (PeproTech). On Day 6, macrophages were treated with 100 ng/ml of rHuEPO. On Day 8, macrophages were harvested, and the intracellular heme level was measured.

### In Vitro Treg Polarization

Fresh mouse CD4⁺ T cells were isolated from spleens using magnetic cell separation against CD4 (Miltenyi Biotec). CD4⁺ T cells were cultured in complete RPMI Medium supplemented with Dynabeads® Mouse T-Activator CD3/CD28 ((Thermo Fisher Scientific) according to manufacturer's recommendation, 5 ng/ml of recombinant mouse IL-2 (PeproTech), 5 ng/ml of recombinant human TGF-β1 (PeproTech) and 100 ng/ml of rHuEPO. On Day 5, cells were harvested for Treg analysis.

### Statistical Analyses

All statistical tests were performed with Prism (GraphPad) and are described as follows except where otherwise noted in figure legends. For all figures, *P<0.05, **P<0.01, ***P<0.001, ****P<0.0001. For most data, replicates are shown as individual points, and where summary data is presented, the number of replicates (n) is noted in the figure legends. All comparisons involving more than two groups utilize tests that adjust for multiple comparisons. Unless otherwise noted, effect sizes are depicted as means (by bar height or horizontal line). Comparisons of two conditions were evaluated using two-tailed paired Student's t test unless otherwise noted. Comparisons of three or more conditions were performed using One-Way ANOVA with Tukey's multiple comparison test, with a single pooled variance. Comparisons of categorical variables were performed with Fisher's Exact test. Comparisons of survival curves were performed using with Log-rank test.

### Example 18—Anti-EPOR Antibody Treatment Leads to Reduction of Tumor in Metastasis Mouse Model

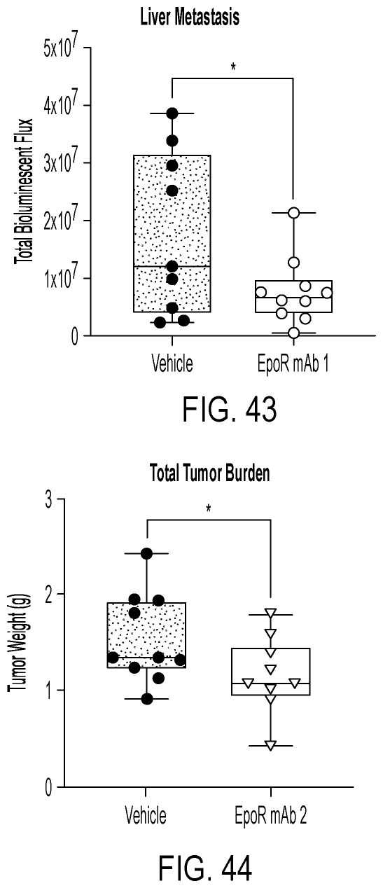

The anti-EPOR antibodies disclosed in Tables 1-1 and 4-20 were tested to study the effect of anti-EPOR antibodies on metastatic tumor burden using two different metastatic cancer mouse models generated for this study and the exemplary results are shown in FIGS. 43 and 44.

First, a spontaneous liver metastasis mouse model was prepared to generate pancreatic metastasis mouse model as described in Example 3, by implanting PLM1 cells into the pancreas of WT C57BL/6 male mice (FIG. 43). Animals (9-10 mice each group) were treated with either vehicle (control) or anti-EPOR mouse antibody 1 (mAb 1) weekly (dose 1). One animal in control group succumbed to tumor burden prior to endpoint. At day 20, primary PDAC and livers were harvested for analysis. Liver metastatic burden was measured ex vivo 21 days post tumor implantation by bioluminescence imaging. As shown in FIG. 43, anti-EPOR antibody treatment led reduction in liver metastases in pancreatic metastasis mouse model.

Second, mouse ovarian tumor cells (BPPNM; p53^{-/-R172H}Brca1^{-/-}Pten^{-/-}Nf1^{-/-}Myc^{OE}) were implanted into the peritoneal cavity of 8-12 week old WT C57BL/6 female mice to generate ovarian metastasis mouse model (FIG. 44). Animals (9-10 mice each group) were treated with either vehicle (control) or anti-EPOR mouse antibody 2 (mAb 2) weekly (dose 1), starting at day 7, via intraperitoneal injection (i.p. injection). At day 28, tumor was harvested for analysis and total tumor burden was measured as total tumor weight. As shown in FIG. 44, anti-EPOR antibody treatment led to reduction of total tumor burden in ovarian metastasis mouse model.

### Example 19—Anti-EPOR Antibody Treatment Demonstrates Long-Term Survival Benefit in Liver Cancer Mouse Model

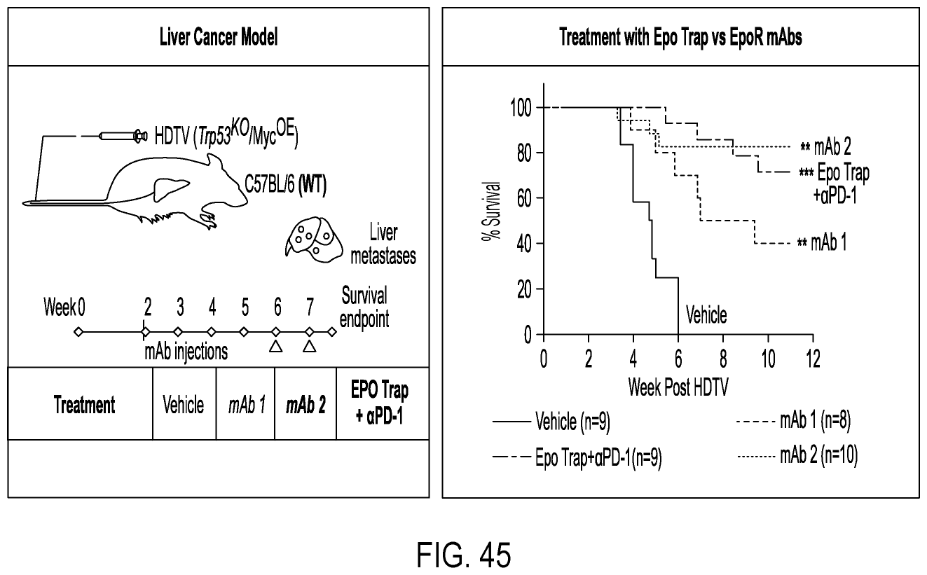

The anti-EPOR antibodies disclosed in Tables 1-1 and 4-20 were tested to study the effect of anti-EPOR antibodies on long-term survival of mice with cancer using liver cancer mouse model and the exemplary results are shown in FIG. 45. Using hydrodynamic tail vein injection (HDTV) method, Trp53^{KO}/Myc^{OE} HCC tumors were injected to WT C57BL/6 male mice. Sterile saline and plasmid mixture were used with a total volume corresponding to 10% of total body weight of mice over 8-10 seconds per animal. As shown in FIG. 45 (left), two weeks after HDTV, animals were treated with vehicle (control), anti-EPOR antibody 1 (mAb 1 at dose 1, once a week), anti-EPOR antibody 2 (mAb 2 at dose 1, once a week), or EPO trap (dose 2, once a week) and anti-PD-1 antibody (αPD-1 at dose 2, twice a week). Efficacy was measured by overall survival of animals. As shown in FIG. 45 (right), treatment with anti-EPOR antibody or with EPO trap and anti-PD1 antibody increased the percentage of survival of mice with liver cancer.

### Sequences

TABLE 1-1
Antibody Sequences

| | SEQ | |
|---|---|---|
| | ID | |
| | NO: | Sequence |
| M2 | 1 | VH: |
| | | QVQLVESGGGLVKPGGSLRLSCAASGFTFSDSYMSWIRQAPGKGLEWVSYISNSGS |
| | | YMYYADSVKGRFTISRDNAKNSLYLQMNNLRAEDTAVYYCARDKLGIGDYWGQGTL |
| | | VTVSS |
| | 2 | VK: |
| | | DIQMTQSPSSLSASVGDRVTITCRASQGIRNDLGWFQQKPGKAPKRLIYAASRLQ |
| | | SGVPSRFSGSGSGTEFTLTISSLOPEDFATYCCLQHHTYPPTFGQGTKVEIK |
| ype464 | 3 | VH: |
| | | EVYLVESGGGLVQPGGSLRLSCEASGFTFSRYWMHWVRQVPGKGLVWVSRINIVG |
| | | STIDYADSVKGRFTISRDNAKNTLYLQMDSLTAEDTAVYYCAREGDRSDAFAIWG |
| | | QGTMVTVSS |
| | 4 | VL: |
| | | SHELTQPLSVSVALGQSAMITCRGNNIGSQNVHWYHQKPGQAPVLVIYRNINRPS |
| | | GIPERFSGSTSGTTATLTISRAQAGDEADYYCQVWDSSGSWVFGGGAKLTVL |

TABLE 2
gRNA oligo pair sequences

| Targeted Genes | |
|---|---|
| (Mouse) | Sequences 5′-3′ |
| Trp53 | Sense: CACCGCCTCGAGCTCCCTCTGAGCC (SEQ ID NO: 81) |
| | Antisense: AAACGGCTCAGAGGGAGCTCGAGGC (SEQ ID |
| | NO: 82) |
| Keap1 | Sense: CACCGGTTCGGTTACCGTCCTGCGA (SEQ ID NO: 83) |
| | Antisense: AAACTCGCAGGACGGTAACCGAACC (SEQ ID |
| | NO: 84) |
| Pten | Sense: CACCGGCTAACGATCTCTTTGATGA (SEQ ID NO: 85) |
| | Antisense: AAACTCATCAAAGAGATCGTTAGCC (SEQ ID NO: |
| | 86) |
| Epo (sg1) | Sense: CACCGCGACAGTCGAGTTCTGGAG (SEQ ID NO: 87) |
| | Antisense: AAACCTCCAGAACTCGACTGTCGC (SEQ ID NO: |
| | 88) |
| Epo (sg2) | Sense: CACCTGAGGCGTGGGGGAGCACAG (SEQ ID NO: 89) |
| | Antisense: AAACCTGTGCTCCCCCACGCCTCA (SEQ ID NO: |
| | 90) |
| Epo (sg3) | Sense: CACCCAAGGAGGCAGAAAATGTCA (SEQ ID NO: 91) |
| | Antisense: AAACTGACATTTTCTGCCTCCTTG (SEQ ID NO: |
| | 92) |

TABLE 3
Sequences of the qPCR primer pairs. (F: Forward primer; R: Reverse primer)

| Targeted Genes (Mouse) | Sequences 5′-3′ |
|---|---|
| 18S | F: ACATCGACCTCACCAAGAGG (SEQ ID NO: 93) |
| | R: TCCCATCCTTCACATCCTTC (SEQ ID NO: 94) |
| Tnf | F: GCCAACGGCATGGATCTCAA (SEQ ID NO: 95) |
| | R: ATAGCAAATCGGCTGACGGT (SEQ ID NO: 96) |
| Il1b | F: GCCACCTTTTGACAGTGATG (SEQ ID NO: 97) |
| | R: ATGTGCTGCTGCGAGATTTG (SEQ ID NO: 98) |
| Ciita | F: CCGTGTAAATGAGACAGAGGAC (SEQ ID NO: 99) |
| | R: GTAGGGAGTGCTGGAATCGC (SEQ ID NO: 100) |
| Cd74 | F: GCCACCACTGCTTACTTCCT (SEQ ID NO: 101) |
| | R: CACAGGTTTGGCAGATTTCGG (SEQ ID NO: 102) |
| H2-Eb1 | F: GACGCCGAGAACTGGAACAG (SEQ ID NO: 103) |
| | R: GTCACCGTAGGCTCAACTCTC (SEQ ID NO: 104) |
| H2-Ab1 | F: GCTTGAACAGCCCAATGTCG (SEQ ID NO: 105) |
| | R: CCAGCGCACTTTGATCTTGG (SEQ ID NO: 106) |
| Cd163 | F: GCGTAGTCTGCTCACGATACA (SEQ ID NO: 107) |
| | R: AGCATCTTCCATGTCCCAATGA (SEQ ID NO: 108) |
| Hmox1 | F: GCCACCAAGGAGGTACACAT (SEQ ID NO: 109) |
| | R: AGGAAGCCATCACCAGCTTA (SEQ ID NO: 110) |
| Epor | F: ACCCCAAGTTTGAGAGCAAAGC (SEQ ID NO: 111) |
| | R: CCTCCCAGAAACACACCAAGTC (SEQ ID NO: 112) |
| Csf2rb | F: ACGATGGGATCTGGAGCAAG (SEQ ID NO: 113) |
| | R: ACACAGCCAAAGCGAAGGAT (SEQ ID NO: 114) |
| Vcam1 | F: TACTGTTTGCAGTCTCTCAAGC (SEQ ID NO: 115) |
| | R: CGTCCTCACCTTCGCGTTTA (SEQ ID NO: 116) |
| Aco1 | F: CCTGTCCCAGTTAGGGTTTGA (SEQ ID NO: 117) |
| | R: CCAACAGCTACGAGGTCTCC (SEQ ID NO: 118) |
| Aco2 | F: CCTGGGAGGCATCTGCATT (SEQ ID NO: 119) |
| | R: GCTTCACACCGATCACCTTGG (SEQ ID NO: 120) |
| Slc40al | F: ATGGATGGGTCCTTACTGTCTG (SEQ ID NO: 121) |
| | R: CCAGTCCCTTTGGATTGTGA (SEQ ID NO: 122) |
| Slc48a1 | F: GCCATCACCCAGCATCAGAG (SEQ ID NO: 123) |
| | R: GGTGGGCGTAGAGGCTAAGT (SEQ ID NO: 124) |
| Gpx1 | F: CGGGACTACACCGAGATGAAC (SEQ ID NO: 125) |
| | R: TCATTCTTGCCATTCTCCTGGT (SEQ ID NO: 126) |
| Gpx4 | F: TACAGGGGTTTCGTGTGCAT (SEQ ID NO: 127) |
| | R: GGGAAGGCCAGGATTCGTAA (SEQ ID NO: 128) |
| Gpx7 | F: ATGTTTAGCAAGATCGCAGTCAC (SEQ ID NO: 129) |
| | R: TCCGTCTGGGTCCACTAGGT (SEQ ID NO: 130) |
| Sod1 | F: GGTTCCACGTCCATCAGTATG (SEQ ID NO: 131) |
| | R: GGTCTCCAACATGCCTCTCT (SEQ ID NO: 132) |
| Sod2 | F: GCTGGCTTGGCTTCAATAAGG (SEQ ID NO: 133) |
| | R: GTGCTCCCACACGTCAATCC (SEQ ID NO: 134) |
| Sod3 | F: GCTTCGACCTAGCAGACAGG (SEQ ID NO: 135) |
| | R: GTGGCTGATGGTTGTACCCT (SEQ ID NO: 136) |
| Cat | F: TGCCGTTCGATTCTCCACAG (SEQ ID NO: 137) |
| | R: CCCACAAGATCCCAGTTACCA (SEQ ID NO: 138) |
| Gstt1 | F: AAGAACAATATCCCGTTCCAGATG (SEQ ID NO: 139) |
| | R: GAGCAAGATAGCCACACTCTCAC (SEQ ID NO: 140) |
| Gstm1 | F: GCTACCTTGCCCGAAAGCA (SEQ ID NO: 141) |
| | R: GCTCCCCAGCAAAGGGTTT (SEQ ID NO: 142) |
| Gstm5 | F: AACGGTACATCTGTGGGGAAG (SEQ ID NO: 143) |
| | R: TCCATGAGGTAGGGCAGGTTA (SEQ ID NO: 144) |
| Gsto1 | F: GGGAAGCGTTGGAGAACGAA (SEQ ID NO: 145) |
| | R: GTGTGGGCTAGACACTCCTTG (SEQ ID NO: 146) |

TABLE 4
Full Heavy Chain (HC) and Light Chain (LC) Sequences for Anti-EPOR Antibodies

| | SEQ | | SEQ | |
|---|---|---|---|---|
| clonotype | ID | | ID | |
| _id | NO | Full HC AA sequence | NO | Full LC AA sequence |
| clonotype | 147 | QVQLQQSGPGLVKPSQTLSLTCAI | 335 | QSALTQPASVSGSPGQSITISC |
| 10 | | SGDSVSSNSAAWNWIRQSPSRGLE | | TGTSSDVGGYNYVSWYQQHPGK |
| | | WLGRTYYRSKWYNDYAVSVKSRIT | | APKLMIYEVSNRPSGVSNRFSG |
| | | INPDTSKNQFSLQLNSVTPEDTAV | | SKSGNTASLTISGLQAEDEADY |
| | | YYCARKGELLGAFDIWGQGTMVTV | | YCSSYTSSSTWVFGGGTKLTVL |
| | | SS | | |
| clonotype | 148 | QVQLQQSGPGLVKPSQTLSLTCAI | 336 | QSALTQPASVSGSPGQSITISC |
| 13 | | SGDSVSSNSAAWNWIRQSPSRGLE | | TGTSSDVGGYNYVSWYQQHPGK |
| | | WLGRTYYRSKWYNDYAGSVKSRII | | APKLMIYDVSKRPSGVSNRFSG |
| | | IIPDTSKNQLSLQLKSVTPEDTAV | | SKSGNTASLTISGLQAEDEADY |
| | | YYCARKWELRDAFDIWGQGTMVTV | | YCCSYAGRSTLGIDWVFGGGTK |
| | | SS | | VTVL |
| clonotype | 149 | EVQLVESGGSVVRPGGSLRLSCAA | 337 | SYELTQPPSVSVSPGQTASITC |
| 22 | | SGFTFDDYGMSWVRQAPGKGLEWV | | SGDKLGDKYACWYQQKPGQSPV |
| | | SGINWNGGSTGYADSVKGRFTISR | | LVIYQDSKRPSGIPERFSGSNS |
| | | DNAKNSLYLQMNSLRAEDTALYYC | | GNTATLTISGTQAMDEADYYCQ |
| | | AREDYGDPGWFDPWGQGTLVTVSS | | AWDSSTYVFGTGTKVTVL |
| clonotype | 150 | QLQLQESGPGLVKPSETLSLTCTV | 338 | SYELTQPSSVSVSPGQTARITC |
| 31 | | SGGSISSSSYYWGWIRQPPGKGLE | | SGDVLAKKYARWFQQKPGQAPV |
| | | WIGSIYYSGSTYYNPSLKSRVTIS | | LVIYKDSERPSGIPERFSGSSS |
| | | VDTSKNQFSLKLSSVTAADTAVYY | | GTTVTLTISGAQVEDEADYYCY |
| | | CATLTGDGDYWGQGTLVTVSS | | SAADNNLVFGGGTKLTVL |
| clonotype | 151 | EVQLVESGGGLVKPGGSLRLSCAA | 339 | SSELTQDPAVSVALGQTVRITC |
| 33 | | SGFTFSSYSMNWVRQAPGKGLEWV | | QGDSLRSYYASWYQQKPGQAPV |
| | | SSISSSSSYIYYADSVKGRFTISR | | LVIYGKNNRPSGIPDRFSGSSS |
| | | DNAKNSLYLQMNSLRAEDTAVYYC | | GNTASLTITGAQAEDEADYYCN |
| | | ARDRSSSWYSFDYWGQGTLVTVSS | | SRDSSGNHRVFGGGTKLTVL |
| clonotype | 152 | EVQLVESGGGLVTPGGSLRLSCAA | 340 | QSALTQPRSVSGSPGQSVTISC |
| 36 | | SGFTENNYSMNWVRQAPGKGLEWV | | TGTSSDVGGYNYVSWYQQHPGK |
| | | SSISSSSSYIYYADSVKGRFTISR | | APKLMIYDVSKRPSGVPDRESG |
| | | DNAKNSLYLPMISLRAEDTAVYYC | | SKSGNTASLTISGLQAEDEADY |
| | | ARDGITGTTFYFDYWGQGTLVTVS | | YCCSYAGSYTWVFGGGTKLTVL |
| | | S | | |
| clonotype | 153 | QVQLVESGGGVVQPGRSLRLSCAA | 341 | QSALTQPPSASGSPGQSVTISC |
| 42 | | SGFTFSSYGMHWVRQAPGKGLEWV | | TGTSSDVGGYNYVSWYQQHPGK |
| | | AVIWYDGSNKYYADSVKGRFTISR | | APKLMIYEVSKRPSGVPDRESG |
| | | DNSKNTLYLQMNSLRAEDTAVYYC | | SKSGNTASLTVSGLQAEDEADY |
| | | ASIAAAGRDYWGQGTLVTVSS | | YCSSYAGSNNLVFGGGTKLTVL |
| clonotype | 154 | EVQLVESGGGLVQPGGSLRLSCAA | 342 | QSALTQPASVSGSPGQSITISC |
| 43 | | SGFTFSSYAMSWVRQAPGKGLEWV | | TGTSSDVGGYNYVSWYQQHPGK |
| | | SAISGSGGSTYYADSVKGRFTISR | | APKLMIYEVSNRPSGVSNRFSG |
| | | DNSKNTLYLQMNSLRAEDTAVYYC | | SKSGNTASLTISGLQAEDEADY |
| | | AKAPELRFDYWGQGTLVTVSS | | YCSSYTSSSTYVFGTGTKVTVL |
| clonotype | 155 | QVQLVQSGAEVKKPGASVKVSCKA | 343 | QSALTQPASVSGSPGQSITISC |
| 44 | | SGYTFTSYGISWVRQAPGQGLEWM | | TGTSSDVGGYNYVSWYQQHPGK |
| | | GWISPYNGNTNYAQNLQDRVTMIT | | APKLMIYDVSKRPSGVSNRFSG |
| | | DTSTTTAYMELRSLRSDDTAVYYC | | SKSGNTASLTISGLQAEDEADY |
| | | ARNHYYYMDVWGKGTTVTVSS | | YCCSYAGSSTYVVFGGGTKLTV |
| | | | | L |
| clonotype | 156 | QVQLQQWGAGLLKPSETLSLTCAV | 344 | SSELTQDPAVSVALGQTVRITC |
| 45 | | YGGSFSGYYWSWIRQPPGKGLEWI | | QGDSLRSYYASWYQQKPGQAPV |
| | | GEINHSGSTNYNPSLKSRVTISVD | | LVIYGKNNRPSGIPDRFSGSSS |
| | | TSKNQFSLKLSSVTAADTAVYYCA | | GNTASLTITGAQAEDEADYYCN |
| | | RGELGIGYWYFDLWGRGTLVTVSS | | SRDSSGNHVVFGGGTKLTVL |
| clonotype | 157 | QVQLVESGGGVVQPGRSLRLSCAA | 345 | SYVLTQPPSVSVAPGKTARITC |
| 47 | | SGFTFSSYGMHWVRQAPGKGLEWV | | GGNNIGSKSVHWYQQKPGQAPV |
| | | AVIWYDGSNKYYADSVKGRFTISR | | LVIYYDSDRPSGIPERFSGSNS |
| | | DNSKNTLYLQMNSLRAEDTAVYYC | | GNTATLTISRVEAGDEADYYCQ |
| | | ARDTGITMVRGVFDYWGQGTLVTV | | VWDSSSDHPVFGGGTKLTVL |
| | | SS | | |
| clonotype | 158 | EVQLVESGGDLVQPGGSLKLSCAA | 346 | QAVLTQPASLSASPGASASLTC |
| 56 | | SGFSFSGSTLHWVRQASGKGLEWI | | TLRSGINVGTYRIYWYQQKPGS |
| | | GHIRSKPNNYATLYGASVKGRFTI | | PPQYLLRYKSDSDKQQGSGVPS |
| | | SRDDSKNTAYLQMNSLKIEDTAVY | | RFSGSKDASANAGILLISGLQS |
| | | YCNGVYGGSSYFFDYWGQGTLVTV | | EDEADYYCMIWHSSAVVFGGGT |
| | | SS | | KLTVL |
| clonotype | 159 | QVQLVQSGAEVKKPGASVKVSCKA | 347 | SSELTQDPAVSVALGQTVRITC |
| 58 | | SGYTFTGYYMHWVRQAPGQGLEWM | | QGDSLRSYYASWYQQKPGQAPV |
| | | GWINPNSGGTNYAQKFQGRVTMTR | | LVIYGKNNRPSGIPDRFSGSSS |
| | | DTSISTAYMELSRLRSDDTAVYYC | | GNTASLTITGAQAEDEADYYCN |
| | | ARDETTIFDYWGQGTLVTVSS | | SRDSSGNWVFGGGTKLTVL |
| clonotype | 160 | QVQLVQSGAEVTKPGASVKVSCKA | 348 | SYELTQPPSVSVSPGQTARITC |
| 62 | | SGYTFINYGISWVRQAPGQGLEWM | | SGDALPKKYAYWYQQKSGQAPV |
| | | GWISAYSGNRNYAQKFQDRVIMTT | | LVIYEDSKRPSGIPERFSGSSS |
| | | DTFTNTAYMELRSLRSDDTAVYYC | | GTMATLTISGAQVEDEADYYCY |
| | | ARLGCNGTSCYTSWYYHFYMDVWG | | STDSSGNHSWVFGGGTKLTVL |
| | | KGTTVTVSS | | |
| clonotype | 161 | QVQLVESGGGVVQPGRSLRLSCAA | 349 | QSALTQPASVSGSPGQSITISC |
| 66 | | SGFTFSSYGMHWVRQAPGKGLEWV | | TGTSSDVGGYNYVSWYQQHPGK |
| | | AVIWYDGSNKYYADSVKGRFTISR | | APKLMIYEVSNRPSGVSNRFSG |
| | | DNSKNTLYLQMNSLRAEDTAVYYC | | SKSGNTASLTISGLQAEDEADY |
| | | ARDEDYYGSGSYSFDYWGQGTLVT | | YCSSYTSSSTLVFGGGTKLTVL |
| | | VSS | | |
| clonotype | 162 | QVQLQESGPGLVKPSGTLSLTCAV | 350 | QAVLTQPASLSASPGASASLTC |
| 69 | | SGGSISSSNWWSWVRQPPGKGLEW | | TLRSGINVGTYRIYWYQQKPGS |
| | | IGEIYHSGSTNYNPSLKSRVTISV | | PPQYLLRYKSDSDKQQGSGVPS |
| | | DKSKNQFSLKLSSVTAADTAVYYC | | RFSGSKDASANAGILLISGLQS |
| | | ARRGAARPFDYWGQGTLVTVSS | | EDEADYYCMIWHSSAYVVFGGG |
| | | | | TKLTVL |
| clonotype | 163 | QITLKESGPTLVKPTQTLTLTCTF | 351 | SYELTQPPSVSVSPGQTASITC |
| 75 | | SGFSLSTSGVGVGWIRQPPGKALE | | SGDKLGDKYACWYQQKPGQSPV |
| | | WLALIYWNDDKRYSPSLKSRLTIT | | LVIYQDSKRPSGIPERFSGSNS |
| | | KDTSKNQVVLTMTNMDPVDTATYY | | GNTATLTISGTQAMDEADYYCQ |
| | | CAHSNWNYGYFDLWGRGTLVTVSS | | AWDSSTAWVFGGGTKLTVL |
| clonotype | 164 | EVQLVESGGGLVQPGGSLRLSCAA | 352 | SYELTQPPSVSVSPGQTARITC |
| 80 | | SGFTFSSYAMSWVRQAPGKGLEWV | | SGDALPKKYAYWYQQKSGQAPV |
| | | SAISGSGGSTYYADSVKGRFTISR | | LVIYEDSKRPSGIPERFSGSSS |
| | | DNSKNTLYLQMNSLRAEDTAVYYC | | GTMATLTISGAQVEDEADYYCY |
| | | AKKDIVATHFDYWGQGTLVTVSS | | STDSSGNHKVFGGGTKLTVL |
| clonotype | 165 | EVQLVESGGGLVKPGGSLRLSCAA | 353 | SSEMTQDPAVSVALGQTVRITC |
| 82 | | SGFTFSNAWMSWVRQAPGKGLEWV | | QGDSLRSYYASWYQQKPGQAPV |
| | | GRIKSKTDGGTTDYAAPVKGRFTI | | LVIYGKNNRPSGIPDRFSGSSS |
| | | SRDDSKNTLYLQMNSLKTEDTAVY | | GNTASLTITGAQAEDEADYYCN |
| | | YCTTADYDFWSGYYMDVWGKGTTV | | SRDSSGNHWVFGGGTKLTVL |
| | | TVSS | | |
| clonotype | 166 | EVQLVESGGGLVQPGGSLRLSCAA | 354 | QSALTQPRSVSGSPGQSVTISC |
| 95 | | SGFTFSSYSMNWVRQAPGKGLEWV | | TGTSSDVGGYNYVSWYQQHPGK |
| | | SYISSSSSTIYYADSVKGRFTISR | | APKLMIYDVSKRPSGVPDRFSG |
| | | DNAKNSLYLQMNSLRDEDTAVYYC | | SKSGNTASLTISGLQAEDEADY |
| | | ARDRYNFDYWGQGTLVTVSS | | YCCSYAGSSWVFGGGTKLTVL |
| clonotype | 167 | EVQLVESGGGVVRPGGSLRLSCAA | 355 | QSALTQPASVSGSPGQSITISC |
| 99 | | SGFTFDDYGMSWVRQAPGKGLEWV | | TGTSSDVGGYNYVSWYQQHPGK |
| | | SGINWNGGSTGYADSVKGRFTISR | | APKLMIYEVSNRPSGVSNRFSG |
| | | DNAKNSLYLQMNSLRAEDTALYYC | | SKSGNTASLTISGLQAEDEADY |
| | | ARGGDTAMVTVFDYWGQGTLVTVS | | YCSSYTSSSTLVFGTGTKVTVL |
| | | S | | |
| clonotype | 168 | EVQLVQSGAEVKKPGESLKISCKG | 356 | QSALTQPASVSGSPGQSITISC |
| 102 | | SGYSFTSYWIGWVRQMSGKGLEWM | | TGTSSDVGGYNYVSWYQQHPGK |
| | | GIIYPSDSDTRYSPSFQGQVTISA | | APKLMIYDVSKRPSGVSNRFSG |
| | | DKSISTAYLQWSSLKASDTAMYYC | | SKSGNTASLTISGLQAEDEADY |
| | | ARQINWGAIDYWGQGTLVTVSS | | YCCSYAGSSTFVVFGGGTKLTV |
| | | | | L |
| clonotype | 169 | QVQLVQSGAEVKKPGASVKVSCKA | 357 | QSALTQPASVSGSPGQSITISC |
| 103 | | SGYTFTSYGISWVRQAPGQGLEWM | | TGTSSDVGGYNYVSWYQQHPGK |
| | | GWISVYNGNTNYAQKLQGRVTMTT | | APKLMIYDVSKRPSGVSNRFSG |
| | | DTSTSTAYMELRSLRSDDTAVYYC | | SKSGNTASLTISGLQAEDEADY |
| | | ARQITATRGFDYWGQGTLVTVSS | | YCCSYAGSSTFVVFGGGTKLTV |
| | | | | L |
| clonotype | 170 | QVQLQQSGPGLVKPSQTLSLTCAI | 358 | QSALTQPASVSGSPGQSITISC |
| 109 | | SGDSVSSNSAAWSWIRQSPSRGLE | | TGTSSDVGGYNYVSWYQQHPGK |
| | | WLGRTYYRSKWYNDYAVSVKSRIT | | APKLMIYDVSKRPSGVSNRFSG |
| | | INPDTSKNQFSLQLNSVTPEDTAV | | SKSGNTASLTISGLQAEDEADY |
| | | YYCARKWELRDTFDIWGQGTMVTV | | YCCSYAGSSTLGIDWVFGGGTK |
| | | SS | | LTVL |
| clonotype | 171 | QVQLQQWGAGLLKPSETLSLTCAV | 359 | LPVLTQPPSASALLGASIKLTC |
| 110 | | YGGSFSGYYWSWIRQPPGKGLEWI | | TLSSEHSTYTIEWYQQRPGRSP |
| | | GEINHSGSTNYNPSLKSRVTISVD | | QYIMKVKSDGSHSKGDGIPDRF |
| | | TSKNQFSLKLSSVTAADTAVYYCA | | MGSSSGADRYLTFSNLQSDDEA |
| | | SYGDFFDYWGQGTLVTVSS | | EYHCGESHTIDGQVGVVFGGGT |
| | | | | KLTVL |
| clonotype | 172 | EVQLVESGGGLVKPGGSLRLSCAA | 360 | LPVLTQPPSASALLGASIKLTC |
| 111 | | SGFTFSSYSMNWVRQAPGKGLEWV | | TLSSEHSTYTIEWYQQRPGRSP |
| | | SSISSSSSYIYYADSVKGRFTISR | | QYIMKVKSDGSHSKGDGIPDRF |
| | | DNAKNSLYLQMNSLRAEDTAVYYC | | MGSSSGADRYLTFSNLQSDDEA |
| | | ARETELTVMDVWGKGTTVTVSS | | EYHCGESHTIDGQVGWVFGGGT |
| | | | | KLTVL |
| clonotype | 173 | EVQLVESGGGLVKPGGSLRLSCAA | 361 | SSELTQDPAVSVALGQTVRITC |
| 112 | | SGFTFSNAWMSWVRQAPGKGLEWV | | QGDSLRSYYASWYQQKPGQAPV |
| | | GRIKSKTDGGTTDYAAPVKGRFTI | | LVIYGKNNRPSGIPDRFSGSSS |
| | | SRDDSKNTLYLQMNSLKTEDTAVY | | GNTASLTITGAQAEDEADYYCN |
| | | YCTTDWEYYDFWSGYYSPYFDYWG | | SRDSSGNHVVFGGGTKLTVL |
| | | QGTLVTVSS | | |
| clonotype | 174 | QVQLQESGPGLVKPSQTLSLTCTV | 362 | SYVLTQPPSVSVPPGKTARITC |
| 397 | | SGGSISSGGYYWSWIRQHPGKGLE | | GGNNVGSKSVHWYQQKPGQAPV |
| | | WIGYIYYIGITYYNPSLKSRVTIS | | LVIYYDTDRPSGIPERFSGSNS |
| | | VDTSKNQFSLKLSSVTAADTAVYY | | GNTATLSISRVEAGDEADYYCQ |
| | | CARAFDYWGQGTLVTVSS | | VWDSRSDHVVFGGGTKLTVL |
| clonotype | 175 | QVQLQESGPGLVKPSQTLSLTCTV | 363 | SYVLTQPPSVSVAPGKTARITC |
| 398 | | SGGSISSGGYYWSWIRQHPGKGLE | | GGNTFGSKTVHWYQQKPGQAPV |
| | | WIGYFYYSGSTYYNPSLKSRVSIS | | LVIYYDSDRPSGIPERFSGSNS |
| | | VDTSKNQFSLRLSSVTVADTAVYY | | GNTATLTISRVEAGDEADYYCQ |
| | | CVRAFDYWGQGTLVTASS | | VWDLYSAHVVFGGGTNLTVL |
| clonotype | 176 | EVQLVESGGGLVKPGGSLRLSCAA | 364 | SYELTQPPSVSVSPGQTARITC |
| 399 | | SGFTFSNAWMSWVRQAPGKGLEWV | | SGDALPKKYAYWYQQKSGQAPV |
| | | GRIKSKTDGGTTDYAAPVKGRFTI | | LVIYEDSKRPSGIPERFSGSSS |
| | | SRDDSKNTLYLQMNSLKTEDTAVY | | GTMATLTISGAQVEDEADYYCY |
| | | YCTTGANWGQGTLVTVSS | | STDSSGNHWVFGGGTKLTVL |
| clonotype | 177 | QVQLVQSGAEVKKPGASVKVSCKA | 365 | QSALTQPASVSGSPGQSITISC |
| 400 | | SGYTFTSYDINWVRQATGQGLEWM | | TGTSSDVGGYNYVSWYQQHPGK |
| | | GWMNPNSGNTGYAQKFQGRVTMTR | | APKLMIYEVSNRPSGVSNRFSG |
| | | NTSISTAYMELSSLRSEDTAVYYC | | SKSGNTASLTISGLQAEDEADY |
| | | ARVAFDIWGQGTMVTVSS | | YCSSYTSSSTVFGGGTKLTVL |
| clonotype | 178 | QVQLVQSGAEAKKPGASVKVSCMA | 366 | QSALTQPPSASGSPGQSVTISC |
| 401 | | SGYTFTTYGISWVRQAPGQGLEWM | | TGTSSDVGGYNYVSWYQQHPGK |
| | | GWISAYNGNTKYAQKLQGRVTMTT | | APKLMIYEVIKRPSGVPDRFSG |
| | | DTSTRTAYMELRSLRSDDTAVYYC | | SKSGNTASLTVSGLQAEDEADY |
| | | ARQIGDYWGQGTLVTVSS | | YCSAYAGSNNVVFGGGTQLTVL |
| clonotype | 179 | EVQLVESGGGLVQPGGSLRLSCAA | 367 | QSVLTQPPSASGTPGQRVTISC |
| 402 | | SGFTFSSYAMSWVRQAPGKGLEWV | | SGSSSNIGSNTVNWYQQLPGTA |
| | | SAISGSGGSTYYADSVKGRFTISR | | PKLLIYSNNQRPSGVPDRESGS |
| | | DNSKNTLYLQMNSLRAEDTAVYYC | | KSGTSASLAISGLQSEDEADYY |
| | | HQTGEDYWGQGTLVTVSS | | CAAWDDSLNGWVFGGGTKLTVL |
| clonotype | 180 | EVQLVESGGGLVKPGGSLRLSCAA | 368 | SYELTQPPSVSVSPGQTARITC |
| 407 | | SGFTFSNAWMSWVRQAPGKGLEWV | | SADALPKQYAYWYQQKPGQAPV |
| | | GRIKSKSDGGTRDYAAPVKGRFTI | | LVIYKDSERPSGIPERFSGSSS |
| | | SRDDSKNTLYLQMNSLKTEDTAMF | | GTTVTLTISGVQAEDEADYYCQ |
| | | YCTTGGTHWGQGTLVTVSS | | SADSSATWVFGGGTKLTVL |
| clonotype | 181 | QVQLVQSGAEVKKPGASVKVSCKA | 369 | SYELTQPPSVSVSPGQTARITC |
| 408 | | SGYTFTSYDINWVRQATGQGLEWM | | SGDALPKKYAYWYQQKSGQAPV |
| | | GWMNPNSGNTGYAQKFQGRVTMTR | | LVIYEDSKRPSGIPERFSGSSS |
| | | NTSISTAYMELSSLRSEDTAVYYC | | GTMATLTISGAQVEDEADYYCY |
| | | ARRSFLDYWGQGTLVTVSS | | STDSSGNHRVFGGGTKLTVL |
| clonotype | 182 | EVQLVESGGGLVKPGGSLRLSCAA | 370 | SYELTQPPSVSVSPGQTARITC |
| 40 | | SGFTFSNAWMSWVRQAPGKGLEWV | | SADALPKQYAYWYQQKPGQAPV |
| | | GRIKSKTDGGTTDYATPVKGRFTI | | VVIYKDSERPSGIPERFSGSSS |
| | | SRADSKNTLFLQMSSLKTEDTAVY | | GTTVTLTISGVQAEDEADYYCQ |
| | | FCTTGGTNWGQGTLVTVSS | | SLDSSGTYWVFGGGTKLTVL |
| clonotype | 183 | EVQLVESGGGLVKPGGSLRLSCAA | 371 | SYELTQPPSVSVSPGQTASITC |
| 413 | | SGFTFSSYSMNWVRQAPGKGLEWV | | SGDKLGDKYACWYQQKPGQSPV |
| | | SSISSSSSYIYYADSVKGRFTISR | | LVIYQDSKRPSGIPERFSGSNS |
| | | DNAKNSLYLQMNSLRAEDTAVYYC | | GNTATLTISGTQAMDEADYYCQ |
| | | ARESSGFDYWGQGTLVTVSS | | AWDSSTVVFGGGTKLTVL |
| clonotype | 184 | QVQLVQSGAEVKKPGASVKVSCKA | 372 | SYELTQPPSVSVSPGQTASITC |
| 414 | | SGYTFTSYDINWVRQATGQGLEWM | | SGDKLGDKYACWYQQKPGQSPV |
| | | GWMNPNSGNTGYAQKFQGRVTMTR | | LVIYQDSKRPSGIPERFSGSNS |
| | | NTSISTAYMELSSLRSEDTAVYYC | | GNTATLTISGTQAMDEADYYCQ |
| | | ARGSSWFDYWGQGTLVTVSS | | AWDSSTVVFGGGTKLTVL |
| clonotype | 185 | EVQLVESGGGLVKPGGSLRLSCAA | 373 | SYELTQPPSVSVSPGQTASITC |
| 415 | | SGFTFSNAWMSWVRQAPGKGLEWV | | SGDKLGDKYACWYQQKPGQSPV |
| | | GRIKSKTDGGTTDYAAPVKGRFTI | | LVIYQDSKRPSGIPERFSGSNS |
| | | SRDDSKNTLYLQMNSLKTEDTAVY | | GNTATLTISGTQAMDEADYYCQ |
| | | YCTLNWGDYWGQGTLVTVSS | | AWDSSTVVFGGGTKLTVL |
| clonotype | 186 | EVQLVESGGGLVKPGGSLRLSCAA | 374 | SSELTQDPAVSVALGQTVRITC |
| 418 | | SGFTFSSYSMNWVRQAPGKGLEWV | | QGDSLRSYYASWYQQKPGQAPV |
| | | SSISSSSSYIYYADSVKGRFTISR | | LVIYGKNNRPSGIPDRFSGSSS |
| | | DNAKNSLYLQMNSLRAEDTAVYYC | | GNTASLTITGAQAEDEADYYCN |
| | | ARAADAFDIWGQGTMVTVSS | | SRDSSGNHWVFGGGTKLTVL |
| clonotype | 187 | EVQLVESGGGLVQPGGSLRLSCAA | 375 | QSALTQPASVSGSPGQSITISC |
| 419 | | SGFTFSSYDMHWVRQATGKGLEWV | | TGTSSDVGGYNYVSWYQQHPGK |
| | | SAIGTAGDTYYPGSVKGRFTISRE | | APKLMIYDVSKRPSGVSNRFSG |
| | | NAKNSLYLQMNSLRAGDTAVYYCA | | SKSGNTASLTISGLQAEDEADY |
| | | RGGSDAFDIWGQGTMVTVSS | | YCCSYAGSVVFGGGTKLTVL |
| clonotype | 188 | EVQLVESGGGLVKPGGSLRLSCTA | 376 | SSELTQDPAVSVALGQTVRITC |
| 420 | | SEFTERNAWMIWVRQAPGKGLEWV | | QGDSLRSYYASWYQQKPGQAPV |
| | | GRIRSEIDGGTTDYAAPVKGRFTI | | LVIYGKNNRPSGIPDRESGSSS |
| | | SRDDSKDTLYLYMNSLKVEDTAVY | | GNTASLTITGAQAEDEADYYCN |
| | | YCTTDHPYYWGHGTLVTVSS | | SRDSSGNHVVFGGGTKLTVL |
| clonotype | 189 | EVQLVESGGGLVKPGGSLRLSCAA | 377 | SSELTQDPAVSVALGQTVRITC |
| 421 | | SGFTFSNAWMSWVRQAPGKGLEWV | | QGDSLRSYYASWYQQKPGQAPV |
| | | GRIKSKTDGGTTDFTAPVKGRFTI | | LVIYGKNNRPSGIPDRFSGSSS |
| | | SRDDSKNTLYLQMNSLKTEDTAVY | | GNTASLTITGAQAEDEADYYCN |
| | | YCTTDHPYYWGQGTLVTVSS | | SRDSSGNHWVFGGGTKLTVL |
| clonotype | 190 | QVQLVESGGGVVQPGRSLRLSCAA | 378 | QSVLTQPPSVSEAPRQRVTISC |
| 423 | | SGFTFSNYGIHWVRQAPGKGLEWV | | SGSSSNIGNNAVNWYQQLPGKA |
| | | AVIWYDGSNEYYVDSVKGRFIISR | | PKLLIYYDDLLPSGVSDRFSGS |
| | | DNSKNTLYLQMNSLRAEDTALYYC | | KSGTSASLAISGLQSEDEADYY |
| | | ALAVTGFDYWGQGTLVTVSS | | CAAWDDRLNGPVFGGGTKLTVL |
| clonotype | 191 | QVQLVESGGGLVKPGGSLRLSCAA | 379 | QSALTQPASVSGSPGQSITISC |
| 424 | | SGFTFSDYYMSWIRQAPGKGLEWV | | TGTSSDVGGYNYVSWYQQHPGK |
| | | SYISSSGSTIYYPDSVKGRFTISR | | APKLMIYDVSKRPSGVSNRFSG |
| | | DNAKNSLYLQMNSLRAEDTAVYYC | | SKSGNTASLTISGLQAEDETDY |
| | | ARDGAAFDIWGQGTMVTVSS | | YCCSYAGSSTLVFGGGTKLTVL |
| clonotype | 192 | QIQLVQSGAEMKKPGASVKVSCKA | 380 | SYELTQPPSVSVSPGQTASITC |
| 426 | | SGYTFTNYGISWVRQAPGQGLEWM | | SGDKLGDKHACWYQQKPGQSPM |
| | | GWINTYNDKTNFALKVQGRVTMTT | | LVIYQDSKRPSGIPERFSGSNS |
| | | DTSTSTAYMELRSLRSDDTAVYYC | | GNTATLTISGTQPMDEADYYCQ |
| | | ARDRGYSFDYWGQGTLVTVSS | | AWDSSTFGGGTKLTVL |
| clonotype | 193 | QVQLVQSGAEVKKPGASVKVSCKA | 381 | SYELTQPSSVSVSPGQTARITC |
| 427 | | SGYTFTSYGISWVRQAPGQGLEWM | | SGDVLAKKYARWFQQKPGQAPV |
| | | GWISAYNGNTNYAQRLQGRVTMTT | | LVIYKDSERPSGIPERFSGSSS |
| | | DTSTSTAYMELRSLISDDTAVYYC | | GTTVTLTISGAQVEDEADYYCY |
| | | ARNHYYYLDVWGKGTTVTVSS | | SAADNNRVFGGGTKLTVL |
| clonotype | 194 | EVQLVESGGGLVQPGGSLRLSCAA | 382 | SYELTQPSSVSVSPGQTARITC |
| 428 | | SGFTFSSYDMHWVRQATGKGLEWV | | SGDVLAKKYARWFQQKPGQAPV |
| | | SAIGTAGDTYYPGSVKGRFTISRE | | LVIYKDSERPSGIPERFSGSSS |
| | | NAKNSLYLQMNSLRAGDTAVYYCA | | GTTVTLTISGAQVEDEADYYCY |
| | | RVSPTGTTDYWGQGTLVTVSS | | SAADNNLVFGGGTKLTVL |
| clonotype | 195 | EVQLVESGGGLVKPGGSLRLSCAA | 383 | SYELTQPSSVSVSPGQTARITC |
| 429 | | SGFTFSNAWMSWVRQAPGKGLEWV | | SGDVLAKKYARWFQQKPGQAPV |
| | | GRIKSKTDGGTTDYAAPVKGRFTI | | LVIYKDSERPSGIPERFSGSSS |
| | | SRDDSKNTLYLQMNSLKTEDTAVY | | GTTVTLTISGAQVEDEADYYCY |
| | | YCTARPLGDVWGKGTTVTVSS | | SAADNNYVFGTGTKVTVL |
| clonotype | 196 | EVQLVESGGGLVKPGGSLRLSCAA | 384 | SYELTQPSSVSVSPGQTARITC |
| 430 | | SGFTFSNAWMSWVRQAPGKGLEWV | | SGDVLAKKYARWFQQKPGQAPV |
| | | GRIKSKTDGGTTDYAAPVKGRFTI | | LVIYKDSERPSGIPERFSGSSS |
| | | SRDDSKNTLYLQMNSLKTEDTAVY | | GTTVTLTISGAQVEDEADYYCY |
| | | YCTTDNGFDYWGQGTLVTVSS | | SAADNNLVFGGGTKLTVL |
| clonotype | 197 | EVQLVESGGGLVKPGGSLRLSCAA | 385 | SSELTQDPAVSVALGQTVRITC |
| 431 | | SGFTFSSYSMNWVRQAPGKGLEWV | | QGDRLRSYYASWYQQKPGQAPV |
| | | SSISSSSSYIYYADSVKGRFTISR | | LVIYGKNNRPSGIPDRFSGSSS |
| | | DNAKNSLYLQMNSLRAEDTAVYYC | | GNTASLTITGAQAEDEADYHCN |
| | | ARDLISSFDYWGQGTLVTVSS | | SRDSSGNHLVFGGGTKLTVL |
| clonotype | 198 | EVQLVESGGGLVQPGGSLRLSCAA | 386 | SSELTQDPAVSVALGQTVRITC |
| 432 | | SGFTFSSYWMSWVRQAPGKGLEWV | | QGDSLRSYYASWYQQKPGQAPV |
| | | ANIKQDGSEKYYVDSVKGRFTISR | | LVIYGKNNRPSGIPDRFSGSSS |
| | | DNAKNSLYLQMNSLRAEDTAVYYC | | GNTASLTITGAQAEDEADYYCN |
| | | ARRIVGAFDYWGQGTLVTVSS | | SRDSSGNHLVFGGGTKLTVL |
| clonotype | 199 | QVQLIQSGTEVKKPGASVKVSCMA | 387 | SYVLTQPPSVSVAPGKTARITC |
| 434 | | SRYTFTSYYIHWVRQAPGQGLEWM | | GGNNIGSKSVHWYQQKPGQAPV |
| | | GIINPSGGTTGYAQKFQGRVTMTR | | LVIYYDSDRPSGIPERFSGSNS |
| | | DTSTSTVYMELYSLRSEDTAVYYC | | GNTATLTISRVEAGDEADYYCQ |
| | | ARGGWGTMDVWGKGTTVTVSS | | VWDSSSDHVVFGGGTKLTVL |
| clonotype | 200 | EVQLVESGGGLVQPGGSLRLSCAA | 388 | SYELTQPPSVSVSPGQTARITC |
| 435 | | SGFTFSSYSMNWVRQAPGKGLEWV | | SGDALPKKYAYWYQQKSGQAPV |
| | | SYISSSSSTIYYADSVKGRFTISR | | LVIYEDSKRPSGIPERFSGSSS |
| | | DNAKNSLYLQMNSLRDEDTAVYYC | | GTMATLTISGAQVEDEADYYCY |
| | | AREGWELLDYWGQGTLVTVSS | | STDSSGNHRVFGGGTKLTVL |
| clonotype | 201 | EVQLVESGGGLIQPGGSLRLSCAA | 389 | QTVVTQEPSLTVSPGGTVTLTC |
| 436 | | SGLTVSTNYMSWVRQAPGKGLEWV | | ASSTGAVTSGYYPNWFQQKPGQ |
| | | SVLYSGGGTYYADSVKGRFTISRD | | APRALIYSTSNKHSWTPARFSG |
| | | NSKNTLCLQMNSLRAEDTAMYYCA | | SLLGGKAALTLSGVQPEDEAEY |
| | | RDNWDSYFDYWGQGTLVTVSS | | YCLLYYGGARVFGGGTKLTVL |
| clonotype | 202 | EVQLVESGGGVVRPGGSLRLSCAA | 390 | QSALTQPPSASGSPGQSVTISC |
| 437 | | SGFTFDDYGMSWVRQAPGKGLEWV | | TGTSSDVGGYNYVSWYQQHPGK |
| | | SGINWNGGSTGYADSVKGRFTISR | | APKLMIYEVSKRPSGVPDRESG |
| | | DNAKNSLYLQMNSLRAEDTALYYC | | SKSGNTASLTVSGLQAEDEADY |
| | | ARTTVTHMDVWGKGTTVTVSS | | YCSSYAGSNNLVFGGGTKLTVL |
| clonotype | 203 | EVQLVESGGGLIQPGGSLRLSCAA | 391 | QSALTQPASVSGSPGQSITISC |
| 438 | | SGFTVSSNYMSWVRQAPGKGLEWV | | TGTSSDVGGYNYVSWYQQHPGK |
| | | SVIYSGGSTYYADSVKGRFTISRD | | APKLMIYDVSKRPSGVSNRFSG |
| | | NSKNTLYLQMNSLRAEDTAVYYCA | | SKSGNTASLTISGLQAEDEADY |
| | | RDWNYDAFDIWGQGTMVTVSS | | YCCSYAGSSTWVFGGGTKLTVL |
| clonotype | 204 | EVQLVESGGGLVKPGGSLRLSCAA | 392 | QSALTQPASVSGSPGQSITISC |
| 439 | | SGFTFSSYSMNWVRQAPGKGLEWV | | TGTSSDVGGYNYVSWYQQHPGK |
| | | SSISSSSSYIYYADSVKGRFTISR | | APKLMIYDVSKRPSGVSNRFSG |
| | | DNAKNSLYLQMNSLRAEDTAVYYC | | SKSGNTASLTISGLQAEDEADY |
| | | ARGDPGWFDPWGQGTLVTVSS | | YCCSYAGSSTFWVFGGGTKLTV |
| | | | | L |
| clonotype | 205 | EVQLVESGGGLVQPGGSLRLSCAA | 393 | SYELTQPPSVSVSPGQTASITC |
| 442 | | SGFTFSSYWMHWVRQAPGKGLVWV | | SGDKLGDKYACWYQQKPGQSPV |
| | | SRINSDGSSTSYADSVKGRFTISR | | LVIYQDSKRPSGIPERFSGSNS |
| | | DNAKNTLYLQMNSLRAEDTAVYYC | | GNTATLTISGTQAMDEADYYCQ |
| | | ARENWNYWFDPWGQGTLVTVSS | | AWDSSTVVFGGGTKLTVL |
| clonotype | 206 | QVQLQESGPGLVKPSGTLSLTCAV | 394 | SYELTQPPSVSVSPGQTASITC |
| 443 | | SGGSISSNNWWSWVRQPPGKGLEW | | SGDKLGDKYACWYQQRPGQSPV |
| | | IGEIYHSGSTNYNPSLKSRVTISV | | LVIYQDNKRPSGIPERFSGSNS |
| | | DKSKNQFSLKVNSVTAADTAIFYC | | GNTATLTISGTQAMDEADYYCQ |
| | | ARLRPGDSFDYWGLGTLVTVSS | | AWDSSTALVFGGGTKLTVL |
| clonotype | 207 | QVHLVQSGSELKKPGASVKVSCKA | 395 | SYELTQPPSVSVSPGQTASITC |
| 444 | | SGYTFTRNGLNWVRQAPGQGLEWM | | SGDKLGDKYACWYQQKPGQSPV |
| | | GWINTNIGNPTYAQGFTGRFVFSL | | LVIYQDSKRPSGIPERFSGSNS |
| | | DTSVSTAYLQISRLQAEDTAVYYC | | GNTATLTISGTQAMDEADYYCQ |
| | | ARSPNWGLFDYWGQGTLVTVSS | | AWDSSTSGVFGGGTKLTVL |
| clonotype | 208 | EVQLVESGGGLVKPGGSLRLSCAA | 396 | SSELTQDPAVSVALGQTVRITC |
| 445 | | SGFTFSSYSMNWVRQAPGKGLEWV | | QGDSLRSYYASWYQQKPGQAPV |
| | | SSISSSSSYIYYADSVKGRFTISR | | LVIYGKNNRPSGIPDRFSGSSS |
| | | DNAKNSLYLQMNSLRAEDTAVYYC | | GNTASLTITGAQAEDEADYYCN |
| | | ARDRGATGFDYWGQGTLVTVSS | | SRDSSGNHWVFGGGTKLTVL |
| clonotype | 209 | QVQLVQSGAEVKKPGASVKVSCKA | 397 | SYELTQPPSVSVSPGQTARITC |
| 446 | | SGYTFTSYGISWVRQAPGQGLEWM | | SGDALPKKYAYWYQQKSGQAPV |
| | | GWISAYNGNTNYAQKLQGRVTMTT | | LVIYEDSKRPSGIPERFSGSSS |
| | | DTSTSTAYMELRSLRSDDTAVYYC | | GTMATLTISGAQVEDEADYYCY |
| | | ARESGELLGDYWGQGTLVTVSS | | STDSSGNHRVFGGGTKLTVL |
| clonotype | 210 | EVQLVESGGGLVQPGGSLRLSCAA | 398 | SSELTQDPAVSVALGQTVRITC |
| 448 | | SGFTFSSYDMHWVRQATGKGLEWV | | QGDSLRSYYASWYQQKPGQAPV |
| | | SAIGTAGDTYYPGSVKGRFTISRE | | LVIYGKNNRPSGIPDRFSGSSS |
| | | NAKNSLYLQMNSLRAGDTAVYYCA | | GNTASLTITGAQAEDEADYYCN |
| | | RYSGSYYYFDYWGQGTLVTVSS | | SRDSSGNHVVFGGGTKLTVL |
| clonotype | 211 | QVQLVESGGGVVQPGRSLRLSCAA | 399 | SYELTQPPSVSVSPGQTARITC |
| 450 | | SGFTFSSYGMHWVRQAPGKGLEWV | | SGDALPKKYAYWYQQKSGQAPV |
| | | AVIWYDGSNKYYADSVKGRFTISR | | LVIYEDSKRPSGIPERFSGSSS |
| | | DNSKNTLYLQMNSLRAEDTAVYYC | | GTMATLTISGAQVEDEADYYCY |
| | | ARGIAAAGKDYWGQGTLVTVSS | | STDSSGNHAVFGGGTQLTVL |
| clonotype | 212 | EVQLVQSGAEVKKPGESLKISCKG | 400 | SYVLTQPPSVSVAPGKTARITC |
| 451 | | SGYSFTSYWIGWVRQMPGKGLEWM | | GGNNIGSKSVHWYQQKPGQAPV |
| | | GIIYPGDSDTRYSPSFQGQVTISA | | LVIYYDSDRPSGIPERFSGSNS |
| | | DKSISTAYLQWSSLKASDTAMYYC | | GNTATLTISRVEAGDEADYYCQ |
| | | ARQDSNYVFDYWGQGTLVTVSS | | VWDSSSDHVVFGGGTKLTVL |
| clonotype | 213 | EVQLVESGGGLVQPGGSLRLSCAA | 401 | QSVLTQSPSAFGTPGQRVTISC |
| 452 | | SGFTFSNYWMSWVRQAPGKGLEWV | | SGSISNLGSNTVNWYQQLPGTA |
| | | ANIKYDGREQYYVDSVKGRFAISR | | PKLLIYSNNQRPSGVPDRESGS |
| | | DNAKNSLSLQMNSLRAEDTAIYYC | | KSGTSASLAISGLQFEDEADYH |
| | | ARDHSAWSFDYWGQGTLVTVSS | | CATWDDSLNGRVFGGGTKLTVL |
| clonotype | 214 | EVQLVESGGGLVQPGGSLRLSCAA | 402 | QSALTQPPSASGSPGQSVTISC |
| 453 | | SGFTFSSYWMSWVRQAPGKGLEWV | | TGTSSDVGGYNYVSWYQQHPGK |
| | | ANIKQDGSEKYYVDSVKGRFTISR | | APKLMIYEVSKRPSGVPDRFSG |
| | | DNAKNSLYLQMNSLRAEDTAVYYC | | SKSGNTASLTVSGLQAEDEADY |
| | | ARRRGSCSFDYWGQGTLVTVSS | | YCSSYAGSNNLVFGGGTKLTVL |
| clonotype | 215 | QVQLVQSGAEVKKPGASVKVSCKA | 403 | QSALTQPPSASGSPGQSVTISC |
| 454 | | SGYTFTSYGISWVRQAPGQGLEWM | | TGTSSDVGGYNYVSWYQQHPGK |
| | | GWISAYNGNTNYAQKLQGRVTMTT | | APKLMIYEVSKRPSGVPDRFSG |
| | | DTSTSTAYMELRSLRSDDTAVYYC | | SKSGNTASLTVSGLQAEDEADY |
| | | ARRSYANCFDYWGQGTLVTVSS | | YCSSYAGSNNWVFGGGTKLTVL |
| clonotype | 216 | EVQLVESGGGLVQPGGSLRLSCAA | 404 | QSALTQPASVSGSPGQSITISC |
| 455 | | SGFTFSSYWMHWVRQAPGKGLVWV | | TGTSSDVGGYNYVSWYQQHPGK |
| | | SRINSDGSSTSYADSVKGRFTISR | | APKLMIYDVSKRPSGVSNRFSG |
| | | DNAKNTLYLQMNSLRAEDTAVYYC | | SKSGNTASLTISGLQAEDEADY |
| | | ARDEQLVPFDIWGQGTMVTVSS | | YCCSYAGSSTLVFGGGTKLTVL |
| clonotype | 217 | EVQLVESGGGLIQPGGSLRLSCAA | 405 | QSALTQPASVSGSPGQSITISC |
| 456 | | SGFTVSSNYMSWVRQAPGKGLEWV | | TGTSSDVGGYNYVSWYQQHPGK |
| | | SVIYSGGSTYYADSVKGRFTISRD | | APKLMIYDVSKRPSGVSNRFSG |
| | | NSKNTLYLQMNSLRAEDTAVYYCA | | SKSGNTASLTISGLQAEDEADY |
| | | RDGAAAGDFQHWGQGTLVTVSS | | YCCSYAGSSTWVFGGGTKLTVL |
| clonotype | 218 | EVQLVESGGGLVQPGRSLRLSCAA | 406 | QSALTQPPSASGSPGQSVTISC |
| 457 | | SGFTEDDYAMHWVRQAPGKGLEWV | | TGTSSDVGGYNYVSWYQQHPGK |
| | | SGISWNSGSIGYADSVKGRFTISR | | APKLMIYEVSKRPSGVPDRESG |
| | | DNAKNSLYLQMNSLRAEDTALYYC | | SKSGNTASLTVSGLQAEDEADY |
| | | AKDSGSYYFDYWGQGTLVTVSS | | YCSSYAGSNNFVVFGGGTKLTV |
| | | | | L |
| clonotype | 219 | QVQLVESGGGLVKPGGSLRLSCAA | 407 | QSVLTQPPSVSGAPGQRVTISC |
| 458 | | SGFTFSDYYMSWIRQAPGKGLEWV | | TGSSSNIGAGYDVHWYQQLPGT |
| | | SYISSSGSTIYYADSVKGRFTISR | | APKLLIYGNSNRPSGVPDRFSG |
| | | DNAKNSLYLQMNSLRAEDTAVYYC | | SKSGTSASLAITGLQAEDEADY |
| | | ARDGQLWSFDYWGQGTLVTVSS | | YCQSYDSSLSDVVFGGGTKLTV |
| | | | | L |
| clonotype | 220 | EVQLVESGGGLIQPGGSLRLSCAA | 408 | QSVLTQPPSVSGAPGQRVTISC |
| 459 | | SGFTVSRNYMSWVRQAPGKGLEWV | | TGSSSNIGAGYDVHWYQQLPGT |
| | | SIIYAGGNTYYADSVKGRFTISRD | | APKLLIYGNNNRPSGVPDRFSG |
| | | NSKNTLYLQMNSLRAEDTGVYYCG | | SKSGTSASLAITGLQAEDEADY |
| | | RVVPIGNWFDPWGQGTLVTVSS | | YCQSYDSSLSGWVFGGGTKLTV |
| | | | | L |
| clonotype | 221 | EVQLVESGGGLVQPGGSLRLSCAA | 409 | QAVLTQPASLSASPGASASLTC |
| 460 | | SGFTFSSYWMSWVRQAPGKGLEWV | | TLRSGINVGTYRIYWYQQKPGS |
| | | ANIKQDGSEKYYVDSVKGRFTISR | | PPQYLLRYKSDSDKQQGSGVPS |
| | | DNAKNSLYLQMNSLRAEDTAVYYC | | RFSGSKDASANAGILLISGLQS |
| | | ARDSNWGVFDYWGQGTLVTVSS | | EDEADYYCMIWHSSAWVFGGGT |
| | | | | KLTVL |
| clonotype | 222 | EVQLVESGGGLVQPGGSLRLSCAA | 410 | QAVLTQPASLSASPGASASLTC |
| 461 | | SGFTFSSYWMSWVRQAPGKGLEWV | | TLRSGINVGTYRIYWYQQKPGS |
| | | ANIKQDGSEKYYVDSVKGRFTISR | | PPQYLLRYKSDSDKQQGSGVPS |
| | | DNAKNSLYLQMNSLRAEDTAVYYC | | RFSGSKDASANAGILLISGLQS |
| | | ARDRLTGDLDYWGQGTLVTVSS | | EDEADYYCMIWHSSAWVFGGGT |
| | | | | KLTVL |
| clonotype | 223 | EVYLVESGGGLVQPGGSLRLSCEA | 411 | SHELTQPLSVSVALGQSAMITC |
| 464 | | SGFTFSRYWMHWVRQVPGKGLVWV | | RGNNIGSQNVHWYHQKPGQAPV |
| | | SRINIVGSTIDYADSVKGRFTISR | | LVIYRNINRPSGIPERFSGSTS |
| | | DNAKNTLYLQMDSLTAEDTAVYYC | | GTTATLTISRAQAGDEADYYCQ |
| | | AREGDRSDAFAIWGQGTMVTVSS | | VWDSSGSWVFGGGAKLTVL |
| clonotype | 224 | EVQLVESGGGLVKPGGSLRLSCAA | 412 | SYELTQPPSVSVSPGQTARITC |
| 465 | | SGFTFSSYSMNWVRQAPGKGLEWV | | SGDALPKKYAYWYQQKSGQAPV |
| | | SSISSSSSYIYYADSVKGRFTISR | | LVIYEDSKRPSGIPERFSGSSS |
| | | DNAKNSLYLQMNSLRAEDTAVYYC | | GTMATLTISGAQVEDEADYYCY |
| | | ARQQWLGYYFDYWGQGTLVTVSS | | STDSSGNHRVFGGGTKLTVL |
| clonotype | 225 | EVQLVESGGGLVQPGGSLRLSCAA | 413 | SSELTQDPALSVALGQTVRITC |
| 466 | | SGFTFSIYWMSWVRQAPGKGLEWV | | QGDSLRSFYASWYQQKPGQAPV |
| | | ATIKEDGSEKYYVDSVKGRFTISR | | LVIYGKSNRPSGIPDRFSGSGS |
| | | DNAKNSLFLQMNSLRADDTAVYYC | | GNTASLTITGAQAEDEADFYCN |
| | | ARDSNFLYYFDYWGQGDLVTVSS | | SRDTSGNYLVFGGGTKLTVL |
| clonotype | 226 | QVQLVQSGAEVKKPGASVKVSCKA | 414 | SSELTQDPAVSVALGQTVRITC |
| 468 | | SGYTFTSYGISWVRQAPGQGLEWM | | QGDSLRSYYASWYQQKPGQAPV |
| | | GWISAYNGNTNYAQKLQGRVTMTT | | LVIYGKNNRPSGIPDRFSGSSS |
| | | DTSTNTAYMELRSLRSDDTAVYYC | | GNTASLTITGAQAEDEADYYCN |
| | | ARQITGTRGFDYWGQGTLVTVSS | | SRDSSGNHWVFGGGTKLTVL |
| clonotype | 227 | QVQLVQSGAEVKKPGASVKVSCKA | 415 | SYELTQPPSVSVSPGQTARITC |
| 469 | | SGYTFTSYDINWVRQATGQGLEWM | | SGDALPKKYAYWYQQKSGQAPV |
| | | GWMNPNSGNTGYAQKFQGRVTMTR | | LVIYEDSKRPSGIPERFSGSSS |
| | | NTSISTAYMELSSLRSEDTAVYYC | | GTMATLTISGAQVEDEADYYCY |
| | | ARMGYSNYPFDYWGQGTLVTVSS | | STDSSGNHVVFGGGTKLTVL |
| clonotype | 228 | QVQLVQSGSEVKKPGASVKVSCKA | 416 | SSELTQDPAVSVALGQTVRITC |
| 470 | | SGYTFTSYYMHWVRQAPGQGLEWM | | QGDSLRSYYASWYQQKPGQAPV |
| | | GIINPSGGSTSYAQKFQGRVTMTR | | LVIYGKNNRPSGIPDRFSGSSS |
| | | DTSTSTVYMELSSLRSEDTAVYYC | | GNTASLTITGAQAEDEADYYCN |
| | | ARGIPTTVTPDYWGQGTLVTVSS | | SRDSSGNHLVFGGGTKLTVL |
| clonotype | 229 | EVQLVESGGGLVQPGGSLRLSCAA | 417 | SYELTQPPSVSVSPGQTARITC |
| 471 | | SGFTFSSYDMHWVRQATGKGLEWV | | SGDALPKKYAYWYQQKSGQAPV |
| | | SAIGTAGDTYYPGSVKGRFTISRE | | LVIYEDSKRPSGIPERFSGSSS |
| | | NAKNSLYLQMNSLRAGDTAVYYCA | | GTMATLTISGAQVEDEADYYCY |
| | | RAGLLTGDAFDIWGQGTMVTVSS | | STDSSGNHRVFGGGTKLTVL |
| clonotype | 230 | EVQLVESGGGLVKPGGSLRLSCAA | 418 | QTVVTQEPSLTVSPGGTVTLTC |
| 474 | | SGFTFSNAWMSWVRQAPGKGLEWV | | ASSTGAVTSGYYPNWFQQKPGQ |
| | | GRIKSKTDGGTTDYAAPVKGRFTI | | TPRALIYSTSNKHSWTPARFSG |
| | | SRDDSKNTLYLQMNSLKTEDTAVY | | SLLGGKAALTLSGVQPEDEAEY |
| | | YCITGTTFPFDYWGQGTLVTVSS | | YCLLYYGGAWVFGGGTKLTVL |
| clonotype | 231 | EVQLVESGEGLVQPGGSLRLSCAA | 419 | QSALTQPASVSGSPGQSITISC |
| 475 | | SGFTFSSHAMHWVRQAPGKGLEYV | | TGTSSDVGGYNYVSWYQHHPGK |
| | | SAISSNGGNTYYADSVKGRFTISR | | APKLMIYEVSNRPSGVSNRFSG |
| | | DNSKNTLYLQVGSLRPEDMAIYYC | | SKSGNTASLTISGLQAEDEADY |
| | | TKGGVGASFDYWGQGTLVTVSS | | YCSSYTSSSTWVFGGGTKLTVL |
| clonotype | 232 | EVQLVESGGGLVKPGGSLRLSCAA | 420 | QSVLTQPPSASGTPGQRVTISC |
| 476 | | SGFTFSSYSMNWVRQAPGKGLEWV | | SGSSSNIGSNTVNWYQQLPGTA |
| | | SSISSSSSYIYYADSVKGRFTISR | | PKLLIYSNNQRPSGVPDRESGS |
| | | DNAKNSLYLQMNSLRAEDTAVYYC | | KSGTSASLAISGLQSEDEADYY |
| | | ARGDYSNYYFDYWGQGTLVTVSS | | CAAWDDSLNGWVFGGGTKLTVL |
| clonotype | 233 | QVQLQQWGAGLLKPSETLSLTCAV | 421 | QSALTQPPSASGSPGQSVTISC |
| 477 | | YGGSFSGYYWSWIRQPPGKGLEWI | | TGTSSDVGGYNYVSWYQQHPGK |
| | | GEINHSGSTNYNPSLKSRVTISVD | | APKLMIYEVSKRPSGVPDRFSG |
| | | TSKNQFSLKLSSVTAADTAVYYCA | | SKSGNTASLTVSGLQAEDEADY |
| | | RWEQPWGQGTLVTVSS | | YCSSYAGSNNWVFGGGTKLTVL |
| clonotype | 234 | QVQLVQSGAEVKKPGASVKVSCKA | 422 | QSALTQPASVSGSPGQSITISC |
| 478 | | SGYTFTSYYMHWVRQAPGQGLEWM | | TGTSSDVGGYNYVSWYQQHPGK |
| | | GIINPSGGSTSYAQKFQGRVTMTR | | APKLMIYDVSKRPSGVSNRFSG |
| | | DTSTSTVYMELSSLRSEDTAVYYC | | SKSGNTASLTISGLQAEDEADY |
| | | ARRTGTTHYFDYWGQGTLVTVSS | | YCCSYAGSSTLVFGGGTKLTVL |
| clonotype | 235 | QVQLVESGGGLVKPGGSLRLSCAA | 423 | QSALTQPASVSGSPGQSITISC |
| 479 | | SGFTFSDYYMSWIRQAPGKGLEWV | | TGTSSDVGGYNYVSWYQQHPGK |
| | | SYISSSGSTIYYADSVKGRFTISR | | APKLMIYDVSKRPSGVSNRFSG |
| | | DNAKNSLYLQMNSLRAEDTAVYYC | | SKSGNTASLTISGLQAEDEADY |
| | | ARGLWLGLYFDYWGQGTLVTVSS | | YCCSYAGSSTWVFGGGTKLTVL |
| clonotype | 236 | EVQLVQSGAEVKKPGESLKISCKG | 424 | QSALTQPPSASGSPGQSVTISC |
| 480 | | SGYSFTSYWIGWVRQMPGKGLEWM | | TGTSSDVGGYNYVSWYQQHPGK |
| | | GIIYPGDSDTRYSPSFQGQVTISA | | APKLMIYEVSKRPSGVPDRFSG |
| | | DKSISTAYLQWSSLKASDTAMYYC | | SKSGNTASLTVSGLQAEDEADY |
| | | ARFLGSSYYFDYWGQGTLVTVSS | | YCSSYAGSNNFEVFGGGTKLTV |
| | | | | L |
| clonotype | 237 | EVQLVESGGGLVQPGGSLRLSCAA | 425 | QAVLTQPASLSASPGASASLTC |
| 481 | | SGFTFSSYSMNWVRQAPGKGLEWV | | TLRSGINVGTYRIYWYQQKPGS |
| | | SYISSSSSTIYYADSVKGRFTISR | | PPQYLLRYKSDSDKQQGSGVPS |
| | | DNAKNSLYLQMNSLRDEDTAVYYC | | RFSGSKDASANAGILLISGLQS |
| | | ARGGAAAGAFDIWGQGTMVTVSS | | EDEADYYCMIWHSSAWVFGGGT |
| | | | | KLTVL |
| clonotype | 238 | QVQLQESGPGLVKPSQTLSLTCTV | 426 | SYELTQPPSVSVSPGQTASITC |
| 486 | | SGGSISSGGYYWSWIRQHPGKGLE | | SGDKLGDKYACWYQQKPGQSPV |
| | | WIGYIFYSGSTYYNPSLKSRVTIS | | VVIYQDSKRPSGIPERFSGSNS |
| | | VDTSKKQYSLKLRSVTAADTAVYY | | GNTATLTISGTQAMDEADYYCQ |
| | | CARAEWELLWFDPWGQGTLVTVSS | | AWDSSTVVFGGGTKLTVL |
| clonotype | 239 | QITLKESGPTLVKPTQTLTLTCTF | 427 | SYELTHPPSVSVSPGQTARITC |
| 487 | | SGFSLSTSGVGVGWIRQPPGKALE | | SADALPKQYAYWYQQKPGQAPV |
| | | WLALIYWNDDKRYSPSLKSRLTIT | | LVIYKDSERPSGIPERFSGSSS |
| | | KDTSKNQVVLTMTNMDPVDTATYF | | GTSVTLTISGVQAEDEADYYCQ |
| | | CAHNYFYISGYFYWGQGTLVTVSS | | SANSGTWVFGGGTKLTVL |
| clonotype | 240 | QVQLVESGGGVVQPGRSLRLSCAA | 428 | SYELTQPPSVSVSPGQTASITC |
| 488 | | SGFTFSSYGMHWVRQAPGKGLEWV | | SGDKLGDKYACWYQQKPGQSPV |
| | | AVISYDGSNKYYADSVKGRFTISR | | LVIYQDTKRPSGIPERFSGSNS |
| | | DNSKNTLYLQMNSLRAEDTAVYYC | | GNTATLTISGTQAMDEADYYCQ |
| | | AKDPLRVVNYMDVWGKGTTVTVSS | | AWDSSTVVFGGGTKLTVL |
| clonotype | 241 | QITLKESGPTLVKPTQTLTLTCTF | 429 | SYELTQPPSVSVSPGQTARITC |
| 490 | | SGFSLSTSGVGVGWIRQPPGKALE | | SADALPNQYAYWYQQKPGQAPV |
| | | WLALIYWSDDKRYSPSLKNRLTIT | | LVIYKDSERPSGIPERFSGSSS |
| | | KDTSKNQVVLTMTNMDPLATATYY | | GTTVTLTISGVQAEDEADYYCQ |
| | | CAQTGYNSWSFDYWGQGTLVTVSS | | SADSSGTWVFGGGTKLTVL |
| clonotype | 242 | QVQLVQSGAEVKKPGASVKVSCKA | 430 | SSELTQDPAVSVALGQTVRITC |
| 492 | | SGYPFTSYGINWVRQAPGQGLEWM | | QGDSLRSYYASWYQQKPGQAPV |
| | | GWISAYNSNTNYAEKFQGRVTMTT | | LVIYGKNNRPSGIPDRFSGSSS |
| | | DTSTTTAYMDLRSLRSDDTAVYYC | | GNTASLTITGAQAEDEADYYCN |
| | | AREDAWNYGWFDPWGQGTLVTVSS | | SRDSSGNHVVFGGGTKLTVL |
| clonotype | 243 | QVQLVQSGAEVKKPGASVKVSCKA | 431 | SYELTQPPSVSVSPGQTARITC |
| 493 | | SGYSFTGYDISWVRQAPRQGLEWM | | SGDALPKKYAYWYQQKSGQAPV |
| | | GWISAYNGNTNYAQKFQARVTMTT | | LVIYEDSKRPSGIPERFSGSSS |
| | | DTSTSTAYMELRSLRSDDTAVYYC | | GTMATLTISGAQVEDEADYYCY |
| | | AREILWLGGYFDYWGQGTLVTVSS | | STDSSGNHRVFGGGTKLTVL |
| clonotype | 244 | QVHLVQSGSELKKPGASVKVSCKA | 432 | SSELTQDPAVSVALGQTVRITC |
| 494 | | SGYTFSSYDMNWIRQAPGQGLEWM | | QGDSLRSYYASWYQQKPGQAPV |
| | | GWINTNTGNPTYAQGFTGRFVFSL | | LVIYGKNNRPSGIPDRFSGSSS |
| | | DTSVSTAYLQISSLKAEDTAVYYC | | GNTASLTITGAQAEDEADYYCN |
| | | AREYSSGWYYFDYWGQGALVTVSS | | SRDSSGNHLVFGGGTKLTVL |
| clonotype | 245 | QVQLVQSGAEVKKPGASVKVSCKA | 433 | SYELTQPPSVSVSPGQTARITC |
| 495 | | SGYTFTGYYMHWVRQAPGQGLEWM | | SGDALPKKYAYWYQQKSGQAPV |
| | | GWINPNSGGTNYAQKFQGRVTMTR | | LVIYEDSKRPSGIPERFSGSSS |
| | | DTSISTAYMELSRLRSDDTAVYYC | | GTMATLTISGAQVEDEADYYCY |
| | | ARERIAVAPPFDYWGQGTLVTVSS | | STDSSGNHRVFGGGTKLTVL |
| clonotype | 246 | QVQLVQSGAEVKKPGASVKVSCKA | 434 | SYELTQPPSVSVSPGQTARITC |
| 496 | | SGYTFTSYDINWVRQATGQGLEWM | | SGDALPKKYAYWYQQKSGQAPV |
| | | GWMNPNSGNTGYAQKFQGRVTMTR | | LVIYEDSKRPSGIPERFSGSSS |
| | | NTSISTAYMELSSLRSEDTAVYYC | | GTMATLTISGAQVEDEADYYCY |
| | | ARAGWELPEYFQHWGQGTLVTVSS | | STDSSGNHRVFGGGTKLTVL |
| clonotype | 247 | QVQLVQSGAEVKKPGASVKVSCKA | 435 | SYELTQPPSVSVSPGQTARITC |
| 497 | | SGYTFTSYDINWVRQATGQGLEWM | | SGDALPKKYAYWYQQKSGQAPV |
| | | GWMNPNSGNTGYAQKFQGRVTMTR | | LVIYEDSKRPSGIPERFSGSSS |
| | | NTSISTAYMELSSLRSEDTAVYYC | | GTMATLTISGAQVEDEADYYCY |
| | | ARGGDDYSNLFDYWGQGTLVTVSS | | STDSSGNHRVFGGGTKLTVL |
| clonotype | 248 | QVQLVQSGAEVKKPGSSVKVPCKA | 436 | SYVLTQPPSVSVAPGKTARITC |
| 498 | | SGDTFSNFAINWVRQAPGQGLEWM | | GGNNIGSKSVHWYQQTPGQAPV |
| | | GGIIPIFATANYAQNFQGRVTITA | | LVIYYDSDRPSGIPDRESGSNS |
| | | DESTSAAYMEVSSLRFEDTAVYYC | | GNTATLTISRVEAGDEADYYCQ |
| | | ARTPLGIGRSFDLWGQGTMVTVSS | | VWDSNSDHWVFGGGTKLTVL |
| clonotype | 249 | EVHLVESGGGLVKPGGSLRLSCAA | 437 | SYELTQPPSVSVSPGQTARITC |
| 499 | | SGFTFSNAWMSWVRQAPGKGLEWV | | SADALPKQYAYWYQQKPGQAPV |
| | | GRIKRKTDGGTTDFASPVKGRFTI | | LVIYKDSERPSGIPERFSGSSS |
| | | SRDDSNNTLYLQMNSLKTEDTAVY | | GTTVTLTISGVQAEDEADYYCQ |
| | | YCTTASTVTTGDYWGQGTLVTVSS | | SADSSGTYPVFGGGTKLTVL |
| clonotype | 250 | QVQLQQSGPGLVKPSQTLSLTCAI | 438 | SSELTQDPAVSVALGQTVRITC |
| 501 | | SGDSVSSNSAAWNWIRQSPSRGLE | | QGDSLRSYYASWYQQKPGQAPV |
| | | WLGRTYYRSKWYNDYAVSVKSRIT | | LVIYGKNNRPSGIPDRFSGSSS |
| | | INPDTSKNQFSLQLNSVTPEDTAV | | GNTASLTITGAQAEDEADYYCN |
| | | YYCARERTEIDYWGQGTLVTVSS | | SRDSSGNHWVFGGGTKLTVL |
| clonotype | 251 | EVQLVESGGGLVKPGGSLRLSCAA | 439 | QAVVTQEPSLTVSPGGTVTLTC |
| 502 | | SGFTENNAWMIWVRQAPGKGLEWV | | GSSTGAVTSGHYPYWFQQKPGQ |
| | | GRIKSKTDGGTTDYGAPVKGRFTI | | APRTLIYDTSNKHSWTPARFSG |
| | | SRDDSKNTLYLQMNSLKTEDTAVY | | SLLGGKAALTLSGAQPEDEAEY |
| | | YCTTGRYFDWFDYWGQGTLVIVSS | | YCLLSYSGARVFGGGTKLTVL |
| clonotype | 252 | EVQLVESGGGLVKPGGSLRLSCAA | 440 | QSALTQPPSASGSPGQSVTISC |
| 504 | | SGFTFSNAWMSWVRQAPGKGLEWV | | TGTSSDVGGYNYVSWYQQHPGK |
| | | GRIKSKTDGGTTDYAAPVKGRFTI | | APKLMIYEVSKRPSGVPDRESG |
| | | SRDDSKNTLYLQMNSLKTEDTAVY | | SKSGNTASLTVSGLQAEDEADY |
| | | YCTTASGSYWFDPWGQGTLVTVSS | | YCSSYAGSNNLVFGGGTKLTVL |
| clonotype | 253 | QVQLVESGGGVVQPGRSLRLSCAA | 441 | QSALTQPASVSGSPGQSITISC |
| 505 | | SGFTFSSYGMHWVRQAPGKGLEWV | | TGTSSDVGGYNYVSWYQQHPGK |
| | | AVISYDGSNKYYADSVKGRFTISR | | APKLMIYDVSKRPSGVSNRFSG |
| | | DNSKNTLYLQMNSLRAEDTAVYYC | | SKSGNTASLTISGLQAEDEADY |
| | | AKGNWNYGDAFDIWGQGTMVTVSS | | YCCSYAGSSTYVFGTGTKVTVL |
| clonotype | 254 | QVQLVESGGGVVRPGGSLRLSCAA | 442 | QSALTQPASVSGSPGQSITISC |
| 506 | | SGFTFDDYGMSWVRQAPGKGLEWV | | TGTSSDVGGYNYVSWYQQHPGK |
| | | SGINWNGGSTGYADSVKGRFTISR | | APKLMIYEVSNRPSGVSNRFSG |
| | | DNAKNSLYLQMNSLRAEDTALYYC | | SKSGNTASLTISGLQAEDEADY |
| | | ARENYDFWSGFDPWGQGTLVTVSS | | YCSSYTSSSTVVFGGGTKLTVL |
| clonotype | 255 | QVHLQQSGPGLVKPSQTLSLTCAI | 443 | QSALTQPASVSGSPGQSITISC |
| 507 | | SGDSVSSNSAAWNWIRQSPSRGLE | | TGTSSDVGGYNYVSWYQQHPGK |
| | | WLGRTYYRSKWYNGYAESVKSRIT | | APKLMIYDVSKRPSGVSNRFSG |
| | | INPDTSKNQFSLQLNSVTPEDTAV | | SKSGNTASLTISGLQAEDEADY |
| | | YYCAREDRGFDYWGQGTLVTVSS | | YCCSYAGSSNVVFGGGTNLTVL |
| clonotype | 256 | EVQLVESGGGLVQPGRSLRLSCAA | 444 | QSALTQPASVSGSPGQSITISC |
| 508 | | SGFTFDDYAMHWVRQAPGKGLEWV | | TGTSSDVGGYNYVSWYQQHPGK |
| | | SGISWNSGSIGYADSVKGRFTISR | | APKLMIYDVSKRPSGVSNRFSG |
| | | DNAKNSLYLQMNSLRAEDTALYYC | | SKSGNTASLTISGLQAEDEADY |
| | | AKRAVVTDYYMDVWGKGTTVTVSS | | YCCSYAGSSTFWVFGGGTKLTV |
| | | | | L |
| clonotype | 257 | EVQLVESGGGLVQPGGSLRLSCAA | 445 | QSALTQPPSASGSPGQSVTISC |
| 509 | | SGFTFSSYSMNWVRQAPGKGLEWV | | TGTSSDVGGYNYVSWYQQHPGK |
| | | SYISSSSNTIYYADSVKGRFTISR | | APKLMIYEVSKRPSGVPDRFSG |
| | | DNAKNSLYLQMSSLRDEDTAVYYC | | SKSGNTASLTVSGLQAEDEADY |
| | | ARTSSWSYDAFDIWGQGTMVTVSS | | YCSSYAGSNNFVVFGGGTKLTV |
| | | | | L |
| clonotype | 258 | QVQLVQSGAEVKKPGASVKVSCKA | 446 | SYELTQPPSVSVSPGQTASITC |
| 511 | | SGYTFTSYYMHWVRQAPGQGLEWM | | SGDKLGDKYACWYQQKPGQSPV |
| | | GIINPSGGSTSYAQKFQGRVTMTR | | LVIYQDSKRPSGIPERFSGSNS |
| | | DTSTSTVYMELSSLRSEDTAVYYC | | GNTATLTISGTQAMDEADYYCQ |
| | | ARERGHTVTPYFDYWGQGTLVTVS | | ATEVFGGGTKLTVL |
| | | S | | |
| clonotype | 259 | EVQLVESGGGLVQPGGSLRLSCAA | 447 | SYELTQPSSVSVSPGQTAKITC |
| 512 | | SGFNFSSYNMNWVRQTPGKGLEWV | | SGDVLAKKYARWFQQKPGQVPV |
| | | SYISNTGNTIYYVDSVKGRFTISR | | LVIYKDSERPSGIPERFSGSSS |
| | | DNAKNSLYLQLNSLRDEDTAVYFC | | GATVTLTISGAQVEDEADYYCY |
| | | ARDGPQVGATDFDYWGQGTLVTVS | | SAADNKVFGGGTKLTVL |
| | | S | | |
| clonotype | 260 | EVQLVESGGGLVKPGGSLRLSCAA | 448 | SYELTQPPSVSVSPGQTASITC |
| 513 | | SGFTFSNAWMSWVRQAPGKGLEWV | | SGDKLGDKYACWYQQKPGQSPV |
| | | GRIKRKTDGGTTDYAAPVKGRFTI | | VVIYQDSKRPSGIPERFSGSNS |
| | | SRDDSKNTLYLQMNSLKTEDTAVY | | GNTATLTISGTQAMDEADYYCQ |
| | | YCTTEYSSSENFDYWGQGTLVTVS | | AWDSSTAVFGGGTKLTVL |
| | | S | | |
| clonotype | 261 | EVQLVESGGGLVQPGGSLRLSCAA | 449 | SYELTQPPSVSVSPGQTASITC |
| 514 | | SGFTFSSYWMHWVRQAPGKGLVWV | | SGDKLGDKYACWYQQKPGQSPV |
| | | SRINSDGSSTSYADSVKGRFTISR | | LVIYQDSKRPSGIPERFSGSNS |
| | | DNAKNTLYLQMNSLRAEDTAVYYC | | GNTATLTISGTQAMDEADYYCQ |
| | | ARDLGAARPRGFDYWGQGTLVTVS | | AWDSSTVVFGGGTKLTVL |
| | | S | | |
| clonotype | 262 | EVQLVESGGGLVQPGGSLRLSCAA | 450 | SYELTQPPSVSVSPGQTARITC |
| 515 | | SGFTFSSYAMSWVRQAPGKGLEWV | | SGDALPKKYAYWYQQKSGQAPV |
| | | SAISGSGGSTYYADSVKGRFTISR | | LVIYEDSKRPSGIPERFSGSSS |
| | | DNSKNTLYLQMNSLRAEDTAVYYC | | GTMATLTISGAQVEDEADYYCY |
| | | AKEGDSGYDSAFDIWGQGTMVTVS | | STDSSGNRVFGGGTKLTVL |
| | | S | | |
| clonotype | 263 | QVQLQESGPGLVKPSGTLSLTCAV | 451 | SYELTQPPSVSVSPGQTARITC |
| 517 | | SGGSISSNNWWSWVRQPPGKGLEW | | SGDALPKKYAYWYQQKSGQAPV |
| | | IGEIYHSGSTNYNPSLKSRVTISV | | LVIYEDSKRPSGIPERFSGSSS |
| | | DKSKNQFSLKLSSVTAADTAVYYC | | GTMATLTISGAQVEDEADYYCY |
| | | ARVLNWNYGDAFDIWGQGTMVTVS | | STDSSGNHRGFGGGTKLTVL |
| | | S | | |
| clonotype | 264 | QVQLQESGPGLVKPSGTLSLTCAV | 452 | QSALTQPRSVSGSPGQSVTISC |
| 518 | | SGGSISSSNWWSWVRQPPGKGLEW | | TGTSSDVGGYNYVSWYQQHPGK |
| | | IGEIYHSGSTNYNPSLKSRVTISV | | APKLMIYDVSKRPSGVPDRFSG |
| | | DKSKNQFSLKLSSVTAADTAVYYC | | SKSGNTASLTISGLQAEDEADY |
| | | ARDPSIVGATAFDIWGQGTMVTVS | | YCCSYAQGVVFGGGTKLTVL |
| | | S | | |
| clonotype | 265 | QVQLQESGPGLVKPSGTLSLTCAV | 453 | SSELTQDPAVSVALGQTVRITC |
| 519 | | SGGSIISSNWWSWVRQSPGKGLGW | | QGDSLRSYYASWYQQKPGQAPV |
| | | IGEIYHSGSTTYNPSLKSRVTISV | | LLIYGKNNRPSGIPDRFSGSSS |
| | | DKSKNQFSLKLSSVTAADTALYYC | | GNTASLTITGAQAEDEADYYCN |
| | | ARSHIVGVNGGFDYWGQGTLVTVS | | SRDSSGNHWVFGGGTKLTVL |
| | | S | | |
| clonotype | 266 | EVQLVESGGGLVKPGGSLRLSCAA | 454 | SSELTQDPAVSVALGQTVRITC |
| 520 | | SGFTFSSYSMNWVRQAPGKGLEWV | | QGDSLRSYYASWYQQKPGQAPV |
| | | SSISSSSSYIYYADSVKGRFTISR | | LVIYGKNNRPSGIPDRFSGSSS |
| | | DNAKNSLYLQMNSLRAEDTAVYYC | | GNTASLTITGAQAEDEADYYCN |
| | | ARDRYNWNYRAFDIWGQGTMVTVS | | SRDSSGNHLVFGTGTKVTVL |
| | | S | | |
| clonotype | 26 | EVQLVESGGGLVQPGGSLRLSCAA | 455 | SSELTQDPAVSVALGQTVRITC |
| 522 | | SGFTFSSYWMSWVRQAPGKGLEWV | | QGDSLRSYYASWYQQKPGQAPV |
| | | ANIKQDGSEKYYVDSVKGRFTISR | | LVIYGKNNRPSGIPDRFSGSSS |
| | | DNAKNSLYLQMNSLRAEDTAVYYC | | GNTASLTITGAQAEDEADYYCN |
| | | ARDLGRGTISWFDPWGQGTLVTVS | | SRDSSGNHWVFGGGTKLTVL |
| | | S | | |
| clonotype | 268 | QITLKESGPTLVKATQTLTLTCTF | 456 | SYVLTQPPSVSVAPGKTARITC |
| 523 | | SGFSLNSSGVGVVWIRQPPGKALE | | GGNNIGSKSVHWYQQKPGQAPV |
| | | WLALIYWNGDKRYSQSLKNRLTIT | | LVIYYDSDRPSGIPERFSGSNS |
| | | EDTSKNQVVLAMTNMDPVDTATYY | | GNTATLTISRVEAGDEADYYCQ |
| | | CTQTGYDSRWSFAYWGQGTLVTVS | | VWDSSSDHWVFGGGTKLTVL |
| | | S | | |
| clonotype | 269 | QGQLVQSGAEVKKPGASVKVSCKT | 457 | SSELTQDPAVSVALGQTVRITC |
| 524 | | SGYIFMNYGITWVRHAPGQGLEWM | | QGDSLRSYYASWYQQKPGQAPV |
| | | GWISAYNGNTNYAQKVQGRVTMTT | | LVIYGKNNRPSGIPDRFSGSSS |
| | | DTSTSTANMELRSLRSDDTAVYYC | | GNTASLTITGAQAEDEADYYCN |
| | | AREGQWRGRGWFALWGQGTQVTVS | | SRDSSGNHLVFGGGTKLTVL |
| | | S | | |
| clonotype | 270 | QVQLVQSGSELKKPGASVKVSCKA | 458 | SSELTQDPAVSVALGQTVRITC |
| 526 | | SGYTFTNYAMNWVRQAPGQGLEWM | | QGDSLRSYYASWYQQKPGQAPV |
| | | GWINTNTGKPTYAQGFTGRFVFSL | | LVIYGKNNRPSGIPDRFSGSSS |
| | | DTSVSTAHLQISGLKAEDTAVYYC | | GNTASLTITGAQAEDEADYYCN |
| | | ARERYFEDFHYMDVWGKGTTVTVS | | SRDSSGNHLVFGGGTKLTVL |
| | | S | | |
| clonotype | 271 | QVQLVQSGAEVKKPGASVKVSCKA | 459 | SSELTQDPAVSVALGQTVRITC |
| 527 | | SGYTFTDNYIHWVRRAPGQGLEWM | | QGDSLRSYYASWYQQKPGQAPV |
| | | GWLNPNSGGTNFAQKFQGRVTMTR | | LVIYGKNNRPSGIPDRFSGSNS |
| | | DTSISSVYMILSSLRSDDTAVYYC | | GNTASLTITGAQAEDEADYYCN |
| | | ARSSWLQLTYYFDYWGQGTLVTVS | | SRDSSGNHLLFGGGTKLTVL |
| | | S | | |
| clonotype | 272 | QVQLVQSGAEVKKPGASVKVSCKA | 460 | SSELTQDPGVSVALGQTVRITC |
| 528 | | SGYTFTSYYIHWVRQAPGQGLEWM | | QGDSLRSYYASWYQQKPGQAPV |
| | | GIINPSGGSTSYAQKFQGRVTMTR | | LVMYGKKNRPSGIPDRFSGSSS |
| | | DTSTSTVYMEVSSLRSEDTAVYYC | | GNTASLTITGAQAEDEADYYCK |
| | | AREGLQLGSNWFDPWGQGTLVTVF | | SRDSSGNHVVFGGGTKLTVL |
| | | S | | |
| clonotype | 273 | EVQLVESGGGLVQPGGSLRLSCAA | 461 | SYELTQPPSVSVSPGQTARITC |
| 529 | | SGFTFSSYSMNWVRQAPGKGLEWV | | SGDALPKKYAYWYQQKSGQAPV |
| | | SYISSSSSTIYYADSVKGRFTISR | | LVIYEDSKRPSGIPERFSGSSS |
| | | DNAKNSLYLQMNSLRDEDTAVYYC | | GTMATLTISGAQVEDEADYYCY |
| | | ARNDILTGEDAFDIWGQGTMVTVS | | STDSSGNHRVFGGGTKLTVL |
| | | S | | |
| clonotype | 274 | EVQMVESGGGLVQPGGSLRLSCAA | 462 | SSELTQDPAVSVALGQTVRITC |
| 530 | | SGFTFSNYAMSWVRQAPGKGLGWV | | QGDSFRNYYASWYQQMPGQAPV |
| | | SGISGSGGRTYYADSVKGRFTISR | | LVIYGKNNRPSGIPDRFSGSSS |
| | | DNSKNTLYLQMNSLRAEDTAVYYC | | GNTASLTITGAQAEDEADYYCN |
| | | AKESIIVGATMFDYWGQGTLVTVS | | SRDSSGNHWVFGGGTKLTVL |
| | | S | | |
| clonotype | 275 | QVQLVESGGGVVQPGRSLRLSCAA | 463 | SSELTQDPAVSVALGQTVRITC |
| 531 | | SGFTFSSYGMHWVRQAPGKGLEWV | | QGDSLRSYYASWYQQKPGQAPV |
| | | AVISYDGSNKYYADSVKGRFTISR | | LVIYGKNNRPSGIPDRFSGSSS |
| | | DNSKNTLYLQMNSLRAEDTAVYYC | | GNTASLTITGAQAEDEADYYCN |
| | | AKGIAALGYYYMDVWGKGTTVTVS | | SRDSSGNHLVFGGGTKLTVL |
| | | S | | |
| clonotype | 276 | EVQLVQSGAEMKKPGESLKISCKD | 464 | SSELTQDPAVSVALGQTVRITC |
| 532 | | SGYRFSNYWIGWVRQLPGKGLEWM | | QGDSLRSYYASWYQQKPGQAPV |
| | | GIIYPGDSDTRYSPSFQGQVTISA | | LVIYGKNNRPSGIPDRFSGSSS |
| | | DKSINTAYLQWNSLKASDTAIYYC | | GNTASLTITGAQAEDEADYYCN |
| | | AKRRITGSHNWFDPWGQGTLVTVS | | SRDSSGNHLVFGGGTKLTVL |
| | | S | | |
| clonotype | 277 | QVQLVQSGSELKKPGASVKVSCKA | 465 | QTVVTQEPSLTVSPGGTVTLTC |
| 534 | | SGYTFTSYAMNWVRQAPGQGLEWM | | ASSTGAVTSGYYPNWFQQKPGQ |
| | | GWINTNTGNPTYAQGFTGRFVFSL | | APRALIYSTSNKHSWTPARFSG |
| | | DTSVSTAYLQISSLKAEDTAVYYC | | SLLGGKAALTLSGVQPEDEAEY |
| | | ARGGTIFGVVNFDYWGQGTLVTVS | | YCLLYYGGARVFGGGTKLTVL |
| | | S | | |
| clonotype | 278 | QVQLVESGGGVVQPGRSLRLSCAA | 466 | QSALTQPASVSGSPGQSITISC |
| 537 | | SGFTFSSYVMHWVRQAPGKGLEWV | | TGTSSDVGVYNFVSWYQQHPGK |
| | | AVIWYDGSNKYFADSVKGRFTISR | | APKLMIYDVTKRPSGASERFSG |
| | | DNSNNTLYLQMNSLRAEDTGVYYC | | SKSGSTASLTISGLQAEDEADY |
| | | LSRSGYSAHNDGDYWGQGTLVTVS | | YCCSYAGSSTWVFGGGTKLTVL |
| | | S | | |
| clonotype | 279 | QVQLVQSGAEVKKPGASVKVSCKA | 467 | QSVLTQPPSVSGAPGQRVTISC |
| 538 | | SGYTFTGYYIFWVRQAPGQGLEWM | | TGSSSNIGAGYDVHWYQQLPGT |
| | | GWINPNSGGTNYAQKFQGRVTMTR | | APKILIYVNNNRPSGVPDRFSG |
| | | DTSITTAYMELSRLRHDDTAVYYC | | SKSGTSASLAITGLQAEDEADY |
| | | TKEGLVVRPDWFDPWGQGTLVTVS | | YCQSYDSSLSGPVFGGGTKLTV |
| | | S | | L |
| clonotype | 280 | QVQLQQSGPGLVKPTQTLSLTCAI | 468 | QSALTQPASVSGSPGQSITISC |
| 539 | | SGDSVSSNSAAWNWIRQSPSRGLE | | TGTSSDVGGYNYVSWYQQHPGK |
| | | WLGRTYYRSKWYNDYAVSVRSRIT | | APKLMIYDVSKRPSGVSNRFSG |
| | | INPDTSKNQFSLHLNSVTPEDTAV | | SKSGNTASLTISGLQAEDETDY |
| | | YYCARKGRDVFDIWGQGTMVTVSS | | YCCSYAGSSTYWVFGGGTKLTV |
| | | | | L |
| clonotype | 281 | QVQLVQSGAEVKKPGASVKVSCKA | 469 | QSVLTQPPSVSGAPGQRVTISC |
| 540 | | SGYTFTSYGISWVRQAPGQGLEWM | | TGSSSNIGAGYDVHWYQQLPGT |
| | | GWISAYNGNTNYAQKLQGRVTMTT | | APKLLIYGNSNRPSGVPDRESG |
| | | DTSTSTAYMELRSLRSDDTAVYYC | | SKSGTSASLAITGLQAEDEADY |
| | | AREGSGSYSDAFDIWGQGTMVTVS | | YCQSYDSSLSGSYVFGTGTKVT |
| | | S | | VL |
| clonotype | 282 | QITLKESGPTLVKPTQTLTLTCTF | 470 | QAVLTQPASLSASPGASASLTC |
| 541 | | SGFSITTSGVGVGWIRQPSGKALE | | TLRSGIHVDTSRIYWYQQKPGS |
| | | WLALIYWNDDKRYSPSLKSRLTIT | | PPQYLLRYKSDSDKHQDSGVPS |
| | | KDTSKNQVVLTMTNMDPVDTATYY | | RFSGSKDASTNAGILLISGLQS |
| | | CTHTEYRNTWCVDYWGQGTLVTVS | | EDEADYYCMIWHSSAIVFGGGT |
| | | S | | KLTVL |
| clonotype | 283 | QITLKESGPTLVKPTQTLTLTCTF | 471 | QAVLTQPASLSASPGASASLTC |
| 542 | | SGFSLSTSGVGVGWIRQPPGKALE | | TLRSGINVGSYRIYWFQQRPGS |
| | | WLALIYWNDDKRYSPSLKSRLTIT | | PPQYLLRYKSDSDKQQGSGVPS |
| | | KDTSKNQVVLTMTNMDPVDTATYY | | RFSGSKDASANAGILLISGLQS |
| | | CAHSPYTSGWPFDYWGQGTLVTVS | | EDEADYYCMIWHSSASVFGGGT |
| | | S | | KVTVL |
| clonotype | 284 | QVQLVQSGAEVKKPGASVKVSCKA | 472 | QAVLTQPASLSASPGASASLTC |
| 543 | | SGYTFTSYDINWVRQATGQGLEWM | | TLRSGINVGTYRIYWYQQKPGS |
| | | GWMNPNSGNTGYAQKFQGRVTMTR | | PPQYLLRYKSDSDKQQGSGVPS |
| | | NTSISTAYMELSSLRSEDTAVYYC | | RFSGSKDASANAGILLISGLQS |
| | | ARVSYSSSWSLFDYWGQGTLVTVS | | EDEADYYCMIWHSSAWVFGGGT |
| | | S | | KLTVL |
| clonotype | 285 | QVTLRESGPALVKPTQTLTLTCTF | 473 | SSELTQDPAVSVALGQTVRITC |
| 548 | | SGFSLSTSGMSLSWIRQPPGKALE | | QGDSLRSYYASWYQQKPGQAPV |
| | | WLALIDWDDDQYYSTSLKTRLTIS | | LVIYGKNNRPSGIPDRFSGSSS |
| | | KDTSKNQVVLSMTNMDPVDTATYY | | GNTASLTITGAQAEDEADYYCN |
| | | CARIRGVGALDGFDFWGQGTMVTV | | SRDSSGNHLVFGGGTKLTVL |
| | | SS | | |
| clonotype | 286 | QVQLVQSGAEVKKPGASVKVSCKA | 474 | SSELTQDPAVSVALGQTVRITC |
| 549 | | SGYTFTSYGISWVRQAPGQGLEWM | | QGDSLRSYYASWYQQKPGQAPV |
| | | GWISGYKGNTNCAQELQGRVTITS | | LVIYGKNNRPSGIPDRFSGSSS |
| | | DTSTSTAYMELRSLRSDDTAVYYC | | GNTASLTITGAQAEDEADYYCN |
| | | ARPLDYGDYEGWEDPWGQGTLVTV | | SRDSSGNHLVFGGGTKLTVL |
| | | SS | | |
| clonotype | 287 | QVQLAQSGIEMRKPGASVKVSCRA | 475 | SSELTQDPTVSVALGQTVRITC |
| 550 | | SGDTFTNCGFGWVRQAPGQGLEWM | | QGDSLRSYYASWYQQKPGQAPV |
| | | GWISAYNGNTNYAQKFQGRVTMTT | | LVIYGKNNRPSGIPDRESGSSS |
| | | DTSTSTAYMELRSLRSDDTAVYYC | | GNTASLTITGAQAEDEADYYCN |
| | | AREGRTNYFYYYMDVWGKGTTVTV | | SRDSSGNHWVFGGGTKLTVL |
| | | SS | | |
| clonotype | 288 | EVQLVESGGGLVQPGRSLRLSCAA | 476 | SSELTQDPAVSVALGQTVRITC |
| 552 | | SGFTFDDYAMHWVRQAPGKGLEWV | | QGDSLRSYYASWYQQKPGQAPV |
| | | SGISKNSGSIGYADSVKGRFTISR | | LVIYGKNNRPSGIPDRFSGSSS |
| | | DNAKKSLYLQMNSLRVEDTALYYC | | GNTASLTITGAQAEDEADYYCN |
| | | AKDITASGDYYYMDVWGKGTTVTV | | SRDSSGNHLVFGGGTKLTVL |
| | | SS | | |
| clonotype | 289 | EVQLVESGGGLVQPGGSLRLSCAA | 477 | SSELTPDPAVSVALGQTVRITC |
| 553 | | SGFTFSSYSMNWVRQAPGKGLEWV | | QGDSLRSYYASWYQQKPGQAPI |
| | | SYISSSSSTIYFADSVKGRFTISR | | LVIYHKNNRPSGIPDRESGSSS |
| | | DNAKNSLYLQMNSLRDEDTAVYYC | | GNTASLTITGAQAEDEADYYCN |
| | | ARDRVYNWNDGAFDIWGQGTMVTV | | SRDSSGNHVVFGGGTKLTVL |
| | | SS | | |
| clonotype | 290 | EVQLVESGGGLVQPGGSLRLSCAA | 478 | SSELTQDPAVSVALGQTVRITC |
| 554 | | SGFTFSSYAMSWVRQAPGKGLEWV | | QGDSLRSYYASWYQQKPGQAPV |
| | | SAISGSGGSTYYADSVKGRFTISR | | LVIYGKNNRPSGIPDRFSGSSS |
| | | DNSKNTMYLQMNSLRAEDTAVYYC | | GNTASLTITGAQAEDEADYYCN |
| | | AKDQRYNWNSWYFDLWGRGTLVTV | | SRDSSGNHLVFGGGTKLTVL |
| | | SS | | |
| clonotype | 291 | QVQLVESGGGVVQPGRSLRLSCAA | 479 | SYELTQPPSVSVSPGQTARITC |
| 555 | | SGFTFSSYGMHWVRQAPGKGLEWV | | SGDALPKKYAYWYQQKSGQAPV |
| | | AVIWYDGSNKYYADSVKGRFTISR | | LVIYEDSKRPSGIPERFSGSSS |
| | | DNSKNTLYLQMNSLRAEDTAVYYC | | GTMATLTISGAQVEDEADYYCY |
| | | ARDHGGVTTYNWFDPWGQGTLVTV | | STDSSGNHRVFGGGTKLTVL |
| | | SS | | |
| clonotype | 292 | QVQLVQSGAEVKKPGASVKVSCKA | 480 | QSALTQPPSASGSPGQSVTISC |
| 559 | | SGYTFTGYYMHWVRQAPGQGLEWM | | TGTSSDVGGYNYVSWYQQHPGK |
| | | GWINPNSGGTNYAQKFQGRVTMTR | | APKLMIYEVSKRPSGVPDRFSG |
| | | DTSISTAYMELSRLRSDDTAVYYC | | SKSGNTASLTVSGLQAEDEADY |
| | | ARDRMVRGVLDAFDIWGQGTMVTV | | YCSSYAGSNNVVFGGGTKLTVL |
| | | SS | | |
| clonotype | 293 | EVQLVESGGGLVQPGGSLRLSCAA | 481 | QSALTQPPSASGSPGQSVTISC |
| 560 | | SGFTFSSYSMNWVRQAPGKGLEWV | | TGTSSDVGGYNYVSWYQQHPGK |
| | | SYISSSSSTIYYADSVKGRFTISR | | APKLMIYEVSKRPSGVPDRESG |
| | | DNAKNSLYLQMNSLRDEDTAVYYC | | SKSGNTASLTVSGLQAEDEADY |
| | | VRGYSSGWYNWYFDLWGRGTLVTV | | YCSSYAGSNNLVFGGGTKLTVL |
| | | SS | | |
| clonotype | 294 | QVQLVESGGGLVKPGGSLRLSCAA | 482 | QTVVTQEPSLTVSPGGTVTLTC |
| 561 | | SGFTFSDYYMSWIRQAPGKGLEWV | | ASSTGAVTSGYYPNWFQQKPGQ |
| | | SYISSSGSTIYYADSVKGRFTISR | | APRALIYSTSNKHSWTPARFSG |
| | | DNAKNSLYLQMNSLRAEDTAVYYC | | SLLGGKAALTLSGVQPEDEAEY |
| | | ARKVPGIAAAGAFDYWGQGTLVTV | | YCLLYYGGAQLVFGGGTKLTVL |
| | | SS | | |
| clonotype | 295 | QVQLVQSGSELKKPGASVKVSCKA | 483 | QSVLTQPPSVSGAPGQRVTISC |
| 562 | | SGYTFNSYAMNWVRQAPGLGLEWM | | TGSNSNIGAGYDIHWYQQLPVT |
| | | GWINTNTGNPTYAQGFSGRFVFSL | | APKLLIYGNSNRPSGVPDRFSG |
| | | DTSVNTAYLQISSLQAEDTAVYFC | | SKSGSSASLAITGLQAEDEADY |
| | | ARGGYGYNFWIRFDPWGQGTLVTV | | YCQSYDNSLSGSVFGGGTKLTV |
| | | SS | | L |
| clonotype | 296 | QLQLQESGPGLVKPSETLSLTCTV | 484 | SYELTQPSSVSVSPGQTARITC |
| 568 | | SGGSISRSSYYWGWIRQPPGRGLE | | SGDVLAKKFARWFQQKPGQAPL |
| | | WIGSIYYSGSTYYNPSLKSRVTIS | | LVIYKDSERPSGIPERFSGSNS |
| | | VDTSKNQFSLKLSSVTAADTGVYY | | GTTVTLTISGAQVEDEADYYCY |
| | | CASYWNFDYWGRGTLVTVSS | | SAADNNLVFGGGTKLTVL |
| clonotype | 297 | QVQLQESGPGLVKPSGTLSLTCAV | 485 | SYELTQPPSVSVSPGQTARITC |
| 569 | | SGGSISSSFWLSWVRQPPGKGLEW | | SGDALPKKYAYWYQQKSGQAPV |
| | | IGEIYHSGSTNYNPSLKSRVTISV | | LVIYEDSKRPSGIPERFSGSSS |
| | | DKSKNQFSLKLTSVTAADTAVYSC | | GTMATLTISGAQVEDEADYYCY |
| | | ARVLGYSYGYRRWFDPWGQGTLVT | | STDSSGNHRVFGGGTKLTVL |
| | | VSS | | |
| clonotype | 298 | EVQLVESGGGLVNPGGSLRLSCAA | 486 | SHMLTQPPSVSVAPGTTARITC |
| 573 | | SGFTFSNAWMSWVRQGPGRGLEWV | | GGNNFGSKSVHWYQQKPGQAPV |
| | | GRIKSKSDGETIDYAAPVKGRFSF | | LVIYYDSDRPSGIPERFSGSNS |
| | | SRDDAENTLYLEMNSLKTEDTAVY | | GNTATLTISRVEAGDEADYYCQ |
| | | YCTTEGSFNFYYFMDVWGKGTAVT | | VWDSTSDHYVFGTGTKVTVL |
| | | VSS | | |
| clonotype | 299 | QVQLQESGPGLVKPSETLSLTCTV | 487 | QSALTQPASVSGSPGQSITISC |
| 576 | | SGGSISSYYWSWIRQPPGKGLEWI | | TGTSSDVGAYNYVSWYQQHPGK |
| | | GHIYYSGSTNYNPSLKSRVTISVD | | APKLMIYAVSKRPSGVSYRFSG |
| | | TSKNQFSLKLSSVTAADTAVYYCA | | SKSGNTASLTISGLQAEDEADY |
| | | RDPFYYDFSDYYYMDVWGKGTTVT | | YCCSYAGTISWVFGGGTKLTVL |
| | | VSS | | |
| clonotype | 300 | EVQLVESGGGLVQPGGSLRLSCAA | 488 | QSALTQPASVSGSPGQSITISC |
| 577 | | SGFTFSSYAMSWVRQAPGKGLEWV | | TGTSSDVGGYNYVSWYQQHPGK |
| | | SAISGSGGSTYYADSVKGRFTISR | | APKLMIYDVSKRPSGVSNRFSG |
| | | DNSKNTLYLQMNSLRAEDTAVYYC | | SKSGNTASLTISGLQAEDEADY |
| | | AKNEARDYYGSGSFDYWGQGTLVT | | YCCSYAGSSTYVFGTGTKVTVL |
| | | VSS | | |
| clonotype | 301 | EVQLVESGGGVVRPGGSLRLSCAA | 489 | QSALTQPPSASGSPGQSVTISC |
| 578 | | SGFTFGDFGMSWVRQAPGKGLEWV | | TGTSSDVGGYNYVSWYQQHPGK |
| | | SGINWNGGSTGYADSVKGRFTISR | | APKLMIYEVNKRPSGVPDRLSG |
| | | DNAKNSLYLQMNSLRAEDTALYYC | | SKSGNTASLTVSGLQAEDEADY |
| | | ASLVGATDYYFYYMDVWGKGTTVT | | YCSSYAGSNNWVFGGGTKLTVL |
| | | VSS | | |
| clonotype | 302 | QVQLQQSGPGLVKPSQTLSLTCAI | 490 | QSALTQPASVSGSPGQSITISC |
| 579 | | SGDSVSSNSAAWNWIRQSPSRGLE | | TGTSSDVGGYNYVSWYQQHPGK |
| | | WLGRTYYRSKWYNDYAVSVKSRIT | | APKLMIYDVSKRPSGVSNRFSG |
| | | INPDTSKNQFSLQLNSVTPEDTAV | | SKSGNTASLTISGLQAEDEADY |
| | | YYCARKWELLDAFDIWGQGTMVTV | | YCCSYAGSSTWVFGGGTKLTVL |
| | | SS | | |
| clonotype | 303 | EVQLVESGGGLVQPGGSLRLSCAA | 491 | QSVLTQPPSVSGAPGQRVTISC |
| 581 | | SGFTFSSYSMNWVRQAPGKGLEWV | | TGSSSNIGAGYDVHWYQQLPGT |
| | | SYISSSSSTIYYADSVKGRFTISR | | APKLLIYGNSNRPSGVPDRFSG |
| | | DNAKNSLYLQMNSLRDEDTAVYYC | | SKSGTSASLAITGLQAEDEADY |
| | | AREERDDYSNYGYFQHWGQGTLVT | | YCQSYDSSLSGWVFGGGTKLTV |
| | | VSS | | L |
| clonotype | 304 | QVQLQQSGPGLVKPSQTLSLTCAI | 492 | QSALTQPPSVSGSPGQSVTISC |
| 582 | | SGDSVSSNSATWNWIRQSPSRGLE | | TGTSSDVGSYNRVSWYQQPPGT |
| | | WLGRSYYMSKWYNDYAVSVKSRIT | | APKLMIYDVSNRPSGVPDRFSG |
| | | INPDTSKNQFSLQLNSVTPEDTAV | | SKSGNTASLTISGLQAEDEADY |
| | | YYCARGDWNYGVLDSWGQGTLVTV | | YCSSYTSSSTYVVFGGGTKLTV |
| | | SS | | L |
| clonotype | 305 | QVQLVQSGAEVKKPRASVKVSCEA | 493 | QSILTQPPSVSATPGQRVTISC |
| 583 | | SGYTFTTYGISWVRQAPGQGLEWM | | TGSDSNIGAGYDVHWYQQLPGA |
| | | GWISAYNGNTKYTQKLQGRVAMTT | | VPRLLIHDNIIRPSGVPDRFSG |
| | | DTSTSTAYMEVRSLRSDDTAVYYC | | SKSDTSASLAISGLHAEDEADY |
| | | ARSGYNWNYDYYFMDVWGTGTTVT | | YCQSYDISLSGSVVFGGGTKLT |
| | | VSS | | VL |
| clonotype | 306 | EVQLVESGGGVVRPGGSLRLSCAA | 494 | SYELTQPPSVSVSPGQTASITC |
| 586 | | SGFTEDDYGMSWVRQAPGKGLEWV | | SGDKLGDKYACWYQQKPGQSPV |
| | | SGINWNGGSTGYADSVKGRFTISR | | LVIYQDSKRPSGIPERFSGSNS |
| | | DNAKNSLYLQMNSLRAEDTALYYC | | GNTATLTISGTQAMDEADYYCQ |
| | | ARDGCSSTSCYGNWFDPWGQGTLV | | AWDSSTAVFGGGTKLTVL |
| | | TVSS | | |
| clonotype | 307 | QVQLQQSGPGLVKPSQTLSLTCAI | 495 | SYELTQPPSVSVSPGQTARITC |
| 587 | | SGDSVSSNSAAWNWIRQSPSRGLE | | SGDGLSKKYAYWYQQKSGQAPV |
| | | WLGRTYYRSKWYSDYAVSVKSRIT | | LVIYEDSKRPSGIPERFSGSSS |
| | | INPDTSKNQFSLQLNSVTPEDTAV | | GTMATLTVSGAQVEDEADYYCY |
| | | YYCARVDFGIVGAIDYWGQGTLVT | | STDSSGKIFGGGTKLTVL |
| | | VSS | | |
| clonotype | 308 | EVQLVESGGGLVKPGGSLRLSCAA | 496 | SSELTQDPAVSVALGQTVRITC |
| 588 | | SAFTFSNYNMNWVRQAPGKGLEWV | | QGDSLRSYYASWYQQKPGQAPV |
| | | SSISSSTSYIYYADSVKGRFTVSR | | LVIYGKNNRPSGIPDRFSGSSS |
| | | DNAKNSLYLQMNSLRAEDTAVYYC | | GNTASLTITGAQAEDEADYYCN |
| | | ARDRDDFWSGYSPYFDYWGQGTLV | | SRDSSGNHWVFGGGTKLTVL |
| | | TVSS | | |
| clonotype | 309 | EVQLVESGGGLVKPGGSLRLSCAA | 497 | SSELTQDPAVSVALGQTVRITC |
| 589 | | SGFTFSSYSMNWVRQAPGKGLEWV | | QGDSLRSYYASWYQQKPGQAPV |
| | | SSISSSSSYIYYADSVKGRFTISR | | LVIYGKNNRPSGIPDRFSGSSS |
| | | DNAKNSLYLQMNSLRAEDTAVYYC | | GNTASLTITGAQAEDEADYYCN |
| | | AREKYDILTGYSPYFDYWGQGTLV | | SRDSSGNHWVFGGGTKLTVL |
| | | TVSS | | |
| clonotype | 310 | EVQLVESGGGLVKPGGSLRLSCAA | 498 | SYELTQPPSVSVSPGQTARITC |
| 596 | | SGFTFSNAWMSWVRQAPGKGLEWV | | SGDALPKKYAYWYQQKSGQAPV |
| | | GRIKSKTDGGTTDYAAPVKGRFTI | | LVIYEDSKRPSGIPERFSGSSS |
| | | SRDDSKNTLYLQMNSLKTEDTAVY | | GTMATLTISGAQVEDEADYYCY |
| | | YCTTDQVSGSYGDAFDIWGQGTMV | | STDSSGNHRVFGGGTKLTVL |
| | | TVSS | | |
| clonotype | 311 | EVQMVESGGGLVQPGGSLRLSCAA | 499 | SSELTQDPAVSVALGQTVRITC |
| 598 | | SGFTFSSHVMSWVRQAPGKGLEWV | | QGDSLRSYYASWYQQKPGQAPV |
| | | SVISGSESSTYYADSVKGRFTISS | | LVIYGKNNRPSGIPDRFSGSSS |
| | | DNSKNTLYLQMNSLRAEDTAIYYC | | GNTASLTITGAQAEDEADYYCN |
| | | AKRAGSGTYYRGYYFDYWGQGTLV | | SRDSSGNHWVFGGGTKLTVL |
| | | TVSS | | |
| clonotype | 312 | LVQLVESGGGVVQPGRSLRLSCAA | 500 | SSELTQDPAVSVALGQTVRITC |
| 599 | | SGFTFSSYGMHWVRQAPGKGLEWV | | QGDSLRSYYASWYQQKPGQAPV |
| | | TLIWYDGSNTYYAESVKGRFTISR | | LVIYGKNNRPSGIPDRFSGSSS |
| | | DNSKSTLYLHMNSLRAEDSAVYYC | | GNTASLTITGAQAEDEADYYCN |
| | | AGTYYYDSSGYLNYMDVWGKGTTV | | SRDSSGNHLVFGGGTKLTVL |
| | | TVSS | | |
| clonotype | 313 | QLQLQESGPGLVKPSETLSLTCTV | 501 | SSELTQDPAVSVALGQTVRITC |
| 600 | | SGGSISSSSYYWGWIRQPPGKGLE | | QGDSLRSYYASWYQQKPGQAPV |
| | | WIGSIHYSGSTYYNPSLKSRVTTS | | LVIYGKNNRPSGIPDRESGSSS |
| | | VDTSKNQFSLKLSSVTAADTAVYY | | GNTASLTITGAQAEDEADYYCN |
| | | CASEGPYFDYWGQGTLVTVSS | | SRDSSGNHWVFGGGTKLTVL |
| clonotype | 314 | QVQLVQSGAEVKKPGASVKVSCKA | 502 | QSALTQPASVSGSPGQSITISC |
| 601 | | SGYSFSSYGIGWVRQAPGQGLEWM | | TGTSSDVGGYNYVSWYQQHPGK |
| | | GWISGYNGNTNYAQKFQGRVTMTT | | APKLMIYEVSNRPSGVSNRFSG |
| | | DTSTSTAHMEVKSLRSDDTAAYYC | | SKSGNTASLTISGLQAEDEADY |
| | | ARAYCGGDCYYSNAFDAWGQGTMV | | YCSSYTSSSTVVFGGGTKLTVL |
| | | TVSS | | |
| clonotype | 315 | EVQLVESGGGLVKPGGSLRLSCAA | 503 | QSALTQPASVSGSPGQSITISC |
| 602 | | SGFTFSNAWMSWVRQAPGKGLEWV | | TGTSSDVGGYNYVSWYQQHPGK |
| | | GRIKSKTDGGTTDYAAPVKGRFTI | | APKLMIYDVSKRPSGVSNRFSG |
| | | SRDDSKNTLYLQMNSLKTEDTAVY | | SKSGNTASLTISGLQAEDEADY |
| | | YCTTDRVTIFGLARMDVWGKGTTV | | YCCSYAGSSTWVFGGGTKLTVL |
| | | TVSS | | |
| clonotype | 316 | EVQLVESGGGLVQPGGSLRLSCAA | 504 | SSELTQDPAVSVALGQTVRITC |
| 607 | | SGFTFSTYSVKWVRQAPGKGLEWV | | QGDSLRSYYATWYQQKPGQAPV |
| | | SYISSGSSTIYYADSVKGRFTISR | | LVIYGRNNRPSGIPDRFSGSSS |
| | | DNAKNSLYLQMNSLRDEDTAVYYC | | GNTASLTITGAQAEDEADYYCY |
| | | ARDPTTIFGVVPYYYMDVWGTGTT | | SRDSSGNHLVFGGGTNLTVL |
| | | VTVSS | | |
| clonotype | 317 | EVQLVESGGGLVKPGGSLRLSCAA | 505 | SYVLTQPPSVSVAPGKTARITC |
| 608 | | SGFTFSNAWMSWVRQAPGKGLEWV | | GGNNIGSKSVHWYQQKPGQAPV |
| | | GRIKSKTDGGTTDYAAPVKGRFTI | | LVIYYDSDRPSGIPERFSGSNS |
| | | SRDDSKNTLYLQMNSLKTEDTAVY | | GNTATLTISRVEAGDEADYYCQ |
| | | YCTTDRDYYGSGSYYFDYWGQGTL | | VWDSSSDHRVFGGGTKLTVL |
| | | VTVSS | | |
| clonotype | 318 | QLQLQESGPGLVKPSETLSLTCTV | 506 | SSELTQDPAVSVALGQTVRITC |
| 610 | | SGGSITTRSYYWGWLRQPPGKGLE | | QGDSLRSYYASWYQQKPGQAPV |
| | | WIGTFYYSGNTYYNPSLQSRVSIS | | LVIYGKNNRPSGIPDRFSGSSS |
| | | VDASKNQFSLQLSSVTAADTAVFY | | GNTASLTITGAQAEDEADYYCN |
| | | CAREDLIGNDYWGQGTLVTVSS | | SRDSSGNHLVFGGGTKLTVL |
| clonotype | 319 | QVHLQQSGPGLVKPSQTLSLTCAI | 507 | SSELTQDPAVSVALGQTVRITC |
| 611 | | SGDSVSSNSAAWNWIRQSPSRGLE | | QGDSLRSYYASWYQQKPGQAPV |
| | | WLGRTYYRSKWYNDYAVSVKSRIT | | LVFYGKNKRPSGIPDRFSGSSS |
| | | INSDTSKNQFSLQLNSVTPEDTAV | | GNTASLTITGAQAEDEADYYCN |
| | | YYCSRDRLIVGASYFDLWGRGTLV | | SRDSNGNHWVFGGGTKLSVL |
| | | TVSS | | |
| clonotype | 320 | EVQLVESGGGVVRPGGSLRLSCAA | 508 | QSALTQPASVSGSPGQSITISC |
| 612 | | SGFTFDDYGMSWVRQAPGKGLEWV | | TGTSSDVGGYNYVSWYQQHPGK |
| | | SGINWNGGSTGYADSVKGRFTISR | | APKLMIYDVSKRPSGVSNRFSG |
| | | DNAKNSLYLQMNSLRAEDTALYYC | | SKSGNTASLTISGLQAEDEADY |
| | | AREKAPAHRSSWSWYFDLWGRGTL | | YCCSYAGSSWVFGGGTKLTVL |
| | | VTVSS | | |
| clonotype | 321 | QVQLVHSGAEVKKPGASVKVSCKA | 509 | QPVLTQPPSVSGVPGQRVTISC |
| 613 | | SGYTFTGNYIHWVRQAPGQGLEWM | | TGSSSNIGARYDVHWYQQLPGT |
| | | GWINPTSGVTNYAQKFQGRVTLTR | | APKLLIYGNSNRPSGVPDRESG |
| | | DTSISTAYMELSRLRSDDTAVYYC | | SKSGTSASLAITGLQAEDEADY |
| | | TREGIAAANPGYFYYMDVWGKGTT | | YCQSYDGTLGGWIFGGGTNLTV |
| | | VTVSS | | L |
| clonotype | 322 | QVQLVQSGAEVKKPGASVKVSCKA | 510 | SSELTQDPAVSVALGQTVRITC |
| 616 | | SGYTFTDYYMHWVRQAPGQGLEWM | | QGDSLRSYYASWYQQKPGQAPV |
| | | GWINPNSGGTNYAQKFQGRVTMTR | | LVIYGKNNRPSGIPDRFSGSSS |
| | | DTSISTAYMELSRLRSDDTAVYFC | | GNTASLTITGAQAEDEADYYCN |
| | | ARCDMVRGVIDHYYNYMDVWGKGT | | SRDSSGNHWVFGGGTKLTVL |
| | | TVTVSS | | |
| clonotype | 323 | QMQLQGSGPGLMKPSETLSLTCTV | 511 | QSALTQPASVSGSPGQSITISC |
| 617 | | SGGSISSRSYYWGWIRQPPGKGLE | | TGTSSDVGGYNYVSWYQQHPGK |
| | | WIGSVFYSGSTYYNPSLKSRVTIS | | APKLMIYDVSKRPSGVSNRFSG |
| | | VDTSKNQFSLKVISVTAADTAVYY | | SKSGNTASLTISGLQAEDEADY |
| | | CVRQTYDSWTGYSFFYFDYWGQGT | | YCCSYAGSSWVFGGGTKLTVL |
| | | LVTVSS | | |
| clonotype | 324 | EVQLVESGGGLVQPGGSLKLSCAA | 512 | SYELTQPPSVSVSPGQTASITC |
| 622 | | SGFTFSGSAMHWVRQASGKGLEWV | | SGDKLGDKYACWYQQKPGQSPV |
| | | GRIRSKANSYATAYAASVKGRFTI | | LVIYQDSKRPSGIPERFSGSNS |
| | | SRDDSKNTAYLQMNSLKTEDTAVY | | GNTATLTISGTQAMDEADYYCQ |
| | | YCTRPMITFGGVIVYDAFDIWGQG | | AWDSSTVIFGGGTKLTVL |
| | | TMVTVSS | | |
| clonotype | 325 | QVQLQQWGAGLLKPSETLSLTCAV | 513 | SYELTQPPSVSVSPGQTARITC |
| 625 | | YGGSFSGYYWSWIRQPPGKGLEWI | | SGDALPKKYAYWYQQKSGQAPV |
| | | GEINHSGSTNYNPSLKSRVTISVD | | LVIYEDNKRPSGIPERFSGSSS |
| | | TSKNQFSLKLSSVTAADTAVYYCA | | GTMATLTISGAQVEDEADYYCY |
| | | RGWGSSSWYYFDYWGQGTLVTVSS | | STDSSGNHRVFGGGTKLTVL |
| clonotype | 326 | QVQLQQWGAGLLKPSETLSLTCAV | 514 | SSELTQDPAVSVALGQTVRITC |
| 626 | | YGGSFSGYYWSWIRQPPGKGLEWI | | QGDSLRNYYASWYQQKPGQAPV |
| | | GEINRSGSTNYNPSLKTRVTISVD | | IVIYGKNNRPSGIPDRFSGSSS |
| | | TSKNQFSLQLSSVTAADTAVYYCA | | GNTASLTITGAQAEDEADYYCN |
| | | RGIFGVGGNWFDPWGQGTLVTVSS | | SRDSSGNHLVFGGGTKLTVL |
| clonotype | 327 | QLQLQESGPGLVKPSETLSLTCTV | 515 | QSALTQPPSASGSPGQSVTISC |
| 629 | | SGGSISSSSYYWGWIRQPPGKGLE | | TGTSSDVGGYNYVSWYQQHPGK |
| | | WIGSIYYSGSTYYNPSLKSRVTIS | | APKLMIYEVSKRPSGVPDRFSG |
| | | VDTSKNQFSLKLSSVTAADTAVYY | | SKSGNTASLTVSGLQAEDEADY |
| | | CARYSSSWSGFDYWGQGTLVTVSS | | YCSSYAGSNNFGGGTKLTVL |
| clonotype | 328 | QLQLQESGPGLVKPSETLSLTCTV | 516 | SSELTQDPAVSVALGQTVRITC |
| 630 | | SGGSISSSSYYWGWIRQPPGKGLE | | QGDSLRTYYASWYQQKPGQAPV |
| | | WIGSIYYSGSTYYNPSLKSRVTIS | | LVIYGKNKRPSGIPDRESGSSS |
| | | VDTSKNQFSLKLSSVTAADTAVYY | | GNTASLTITGAQAEDEADYYCN |
| | | CARGGSYYVYFDYWGQGTLVTVSS | | SRDSSGNHWVFGGGTKLTVL |
| clonotype | 329 | KVQLVESGGGLVQPGGSLRLSCAA | 517 | SYVLTQPPSVSVAPGQTARIIC |
| 634 | | SGFTFRSYWMSWVRQAPGKGLEWV | | GGDNIGIKNVHWYQQKPGQAPV |
| | | ANINQDGSEKYYVDSVKGRFTISR | | LVIYDDSDRPSGIPERFSGSNS |
| | | DNAKNSLYLHMNSLRAEDTAVYYC | | GNTATLTISRVEAGDEADYCCQ |
| | | SRDTDCSSTSCYFNWNPFFDYWGQ | | VWDSSSDHVVFGGGTKLTVL |
| | | GTLVTVSS | | |
| clonotype | 330 | QLQLQESGPGLVKPSETLSLTCTV | 518 | SYELTQPPSVSVSPGQTASITC |
| 638 | | SGGSINSSNFYWGWIRQPPGKGLE | | SGDKLGDKYTCWYQQKPGQSPV |
| | | WFGSIFYSGFTYYNPSLKSRVTIS | | LVIYQDIKRPSGIPDRESGSNS |
| | | VDTSKNQFSLKLTSVTAADTAVYY | | GNTATLTISGTQAMDEADYYCQ |
| | | CARGGYSYGLNWFDPWGQGTLVTV | | AWDSSTVVFGGGTKLTVL |
| | | SS | | |
| clonotype | 331 | QLQLQESGPGLVKPSETLSLTCTV | 519 | SSELTQDPAVSVALGQTVRITC |
| 641 | | SGGSISSSSYYWGWIRQPPGKGLE | | QGDSLRSYYASWYQQKPGQAPV |
| | | WIGSIYYSGSTYYNPSLKSRVTIS | | LVIYGKNNRPSGIPDRFSGSSS |
| | | VDTSKNQFSLKLSSVTAADTAVYY | | GNTASLTITGAQAEDEADYYCN |
| | | CARTYYDFWSGYLNWFDPWGQGTL | | SRDSSGNHVVFGGGTKLTVL |
| | | VTVSS | | |
| clonotype | 332 | QVQLQQWGAGLLKPSETLSLTCAV | 520 | QSALTQPPSASGSPGQSVTISC |
| 644 | | YGGSFSGYYWSWIRQPPGKGLEWI | | TGTSSDVGGYNYVSWYQQHPGK |
| | | GEINRGGSTNYNPSLKSRVTISVD | | APKLMIYEVSKRPSGVPDRESG |
| | | TSKNQFSLKLSSVTAADTAVYYCA | | SKSGNTASLTVSGLQAEDEADY |
| | | RWRNYYDSSGSPYWYFDLWGRGSL | | YCSSYAGSNNWVFGGGTKLTVL |
| | | VTVSS | | |
| clonotype | 333 | QLQLQESGPGLVKPSETLSLTCTV | 521 | QSALTQPASVSGSPGQSITISC |
| 646 | | SGGSISSSGYYWGWIRQSPGKGLE | | TGTSSDVGGYNYVSWYQQHPGK |
| | | WIGSFYYSGSTYYNPSLKSRVTIS | | APKLMIYEVSNRPSGVSNRFSG |
| | | VDTSKNQFSLKLSSVTAADTAVYY | | SKSGNTASLTISGLQAEDEADY |
| | | CARQGRITMVRGVIPFDYWGQGTL | | YCSSYTSSSTLVFGGGTKLTVL |
| | | VTVSS | | |
| clonotype | 334 | QVQLQQWGAGLLKPSETLSLTCAV | 522 | QAVVTQEPSLTVSPGGTVTLTC |
| 647 | | YGGSFSGYYWSWIRQPPGKGLEWI | | GSSTGAVTSGHYPYWFQQKPGQ |
| | | GEINHSGSTNYNPSLKSRVTISVD | | APRTLIYDTSNKHSWTPARFSG |
| | | TSKNQFSLKLSSVTAADTAVYYCA | | SLLGGKAALTLSGAQPEDEAEY |
| | | GGYCSSTSCRYNWNYGGWFDPWGQ | | YCLLSYSGARVFGGGTKLTVL |
| | | GTLVTVSS | | |

TABLE 5
VH-CDR and VL-CDR Sequences of Antibodies of Table 4

| clono- | SEQ | HCD | SEQ | HCD | SEQ | HCD | SEQ | LCD | SEQ | LCD | SEQ | LCD |
|---|---|---|---|---|---|---|---|---|---|---|---|---|
| type_ | ID | R1 | ID | R2 | ID | R3 | ID | R1 | ID | R2 | ID | R3 |
| id | NO | AA | NO | AA | NO | AA | NO | AA | NO | AA | NO | AA |
| clono- | 1087 | GDS | 1275 | YYRS | 1463 | CARK | 1651 | TGTS | 1839 | EVS | 2027 | CSSY |
| type10 | | VSS | | KWY | | GELL | | SDVG | | NRP | | TSSS |
| | | NSA | | | | GAFD | | GYNY | | S | | TWVF |
| | | | | | | IW | | VS | | | | |
| clono- | 1088 | GDS | 1276 | YYRS | 1464 | CARK | 1652 | TGTS | 1840 | DVS | 2028 | CCSY |
| type13 | | VSS | | KWY | | WELR | | SDVG | | KRP | | AGRS |
| | | NSA | | | | DAFD | | GYNY | | S | | TLGI |
| | | | | | | IW | | VS | | | | DWVF |
| clono- | 1089 | GFT | 1277 | NWNG | 1465 | CARE | 1653 | SGDK | 1841 | QDS | 2029 | CQAW |
| type22 | | FDD | | GS | | DYGD | | LGDK | | KRP | | DSST |
| | | Y | | | | PGWF | | YAC | | S | | YVF |
| | | | | | | DPW | | | | | | |
| clono- | 1090 | GGS | 1278 | YYSG | 1466 | CATL | 1654 | SGDV | 1842 | KDS | 2030 | CYSA |
| type31 | | ISS | | S | | TGDG | | LAKK | | ERP | | ADNN |
| | | SSY | | | | DYW | | YAR | | S | | LVF |
| clono- | 1091 | GFT | 1279 | SSSS | 1467 | CARD | 1655 | QGDS | 1843 | GKN | 2031 | CNSR |
| type33 | | FSS | | SY | | RSSS | | LRSY | | NRP | | DSSG |
| | | Y | | | | WYSF | | YAS | | S | | NHRV |
| | | | | | | DYW | | | | | | F |
| clono- | 1092 | GFT | 1280 | SSSS | 1468 | CARD | 1656 | TGTS | 1844 | DVS | 2032 | CCSY |
| type36 | | FNN | | SY | | GITG | | SDVG | | KRP | | AGSY |
| | | Y | | | | TTFY | | GYNY | | S | | TWVE |
| | | | | | | FDYW | | VS | | | | |
| clono- | 1093 | GFT | 1281 | WYDG | 1469 | CASI | 1657 | TGTS | 1845 | EVS | 2033 | CSSY |
| type42 | | FSS | | SN | | AAAG | | SDVG | | KRP | | AGSN |
| | | Y | | | | RDYW | | GYNY | | S | | NLVF |
| | | | | | | | | VS | | | | |
| clono- | 1094 | GFT | 1282 | SGSG | 1470 | CAKA | 1658 | TGTS | 1846 | EVS | 2034 | CSSY |
| type43 | | FSS | | GS | | PELR | | SDVG | | NRP | | TSSS |
| | | Y | | | | FDYW | | GYNY | | S | | TYVE |
| | | | | | | | | VS | | | | |
| clono- | 1095 | GYT | 1283 | SPYN | 1471 | CARN | 1659 | TGTS | 1847 | DVS | 2035 | CCSY |
| type44 | | FTS | | GN | | HYYY | | SDVG | | KRP | | AGSS |
| | | Y | | | | MDVW | | GYNY | | S | | TYVV |
| | | | | | | | | VS | | | | F |
| clono- | 1096 | GGS | 1284 | NHSG | 1472 | CARG | 1660 | QGDS | 1848 | GKN | 2036 | CNSR |
| type45 | | FSG | | S | | ELGI | | LRSY | | NRP | | DSSG |
| | | Y | | | | GYWY | | YAS | | S | | NHVV |
| | | | | | | FDLW | | | | | | F |
| clono- | 1097 | GFT | 1285 | WYDG | 1473 | CARD | 1661 | GGNN | 1849 | YDS | 2037 | CQVW |
| type47 | | FSS | | SN | | TGIT | | IGSK | | DRP | | DSSS |
| | | Y | | | | MVRG | | SVH | | S | | DHPV |
| | | | | | | VFDY | | | | | | F |
| | | | | | | W | | | | | | |
| clono- | 1098 | GFS | 1286 | RSKP | 1474 | CNGV | 1662 | TLRS | 1850 | YKS | 2038 | CMIW |
| type56 | | FSG | | NNYA | | YGGS | | GINV | | DSD | | HSSA |
| | | S | | | | SYFF | | GTYR | | KQQ | | VVF |
| | | | | | | DYW | | IY | | GS | | |
| clono- | 1099 | GYT | 1287 | NPNS | 1475 | CARD | 1663 | QGDS | 1851 | GKN | 2039 | CNSR |
| type58 | | FTG | | GG | | ETTI | | LRSY | | NRP | | DSSG |
| | | Y | | | | FDYW | | YAS | | S | | NWVF |
| clono- | 1100 | GYT | 1288 | SAYS | 1476 | CARL | 1664 | SGDA | 1852 | EDS | 2040 | CYST |
| type62 | | FIN | | GN | | GCNG | | LPKK | | KRP | | DSSG |
| | | Y | | | | TSCY | | YAY | | S | | NHSW |
| | | | | | | TSWY | | | | | | VF |
| | | | | | | YHFY | | | | | | |
| | | | | | | MDVW | | | | | | |
| clono- | 1101 | GFT | 1289 | WYDG | 1477 | CARD | 1665 | TGTS | 1853 | EVS | 2041 | CSSY |
| type66 | | FSS | | SN | | EDYY | | SDVG | | NRP | | TSSS |
| | | Y | | | | GSGS | | GYNY | | S | | TLVF |
| | | | | | | YSFD | | VS | | | | |
| | | | | | | YW | | | | | | |
| clono- | 1102 | GGS | 1290 | YHSG | 1478 | CARR | 1666 | TLRS | 1854 | YKS | 2042 | CMIW |
| type69 | | ISS | | S | | GAAR | | GINV | | DSD | | HSSA |
| | | SN | | | | PFDY | | GTYR | | KQQ | | YVVF |
| | | | | | | W | | IY | | GS | | |
| clono- | 1103 | GFS | 1291 | YWND | 1479 | CAHS | 1667 | SGDK | 1855 | QDS | 2043 | CQAW |
| type75 | | LST | | D | | NWNY | | LGDK | | KRP | | DSST |
| | | SGV | | | | GYFD | | YAC | | S | | AWVF |
| | | | | | | LW | | | | | | |
| clono- | 1104 | GFT | 1292 | SGSG | 1480 | CAKK | 1668 | SGDA | 1856 | EDS | 2044 | CYST |
| type80 | | FSS | | GS | | DIVA | | LPKK | | KRP | | DSSG |
| | | Y | | | | THED | | YAY | | S | | NHKV |
| | | | | | | YW | | | | | | F |
| clono- | 1105 | GFT | 1293 | KSKT | 1481 | CTTA | 1669 | QGDS | 1857 | GKN | 2045 | CNSR |
| type82 | | FSN | | DGGT | | DYDF | | LRSY | | NRP | | DSSG |
| | | A | | | | WSGY | | YAS | | S | | NHWV |
| | | | | | | YMDV | | | | | | F |
| | | | | | | W | | | | | | |
| clono- | 1106 | GFT | 1294 | SSSS | 1482 | CARD | 1670 | TGTS | 1858 | DVS | 2046 | CCSY |
| type95 | | FSS | | ST | | RYNF | | SDVG | | KRP | | AGSS |
| | | Y | | | | DYW | | GYNY | | S | | WVF |
| | | | | | | | | VS | | | | |
| clono- | 1107 | GFT | 1295 | NWNG | 1483 | CARG | 1671 | TGTS | 1859 | EVS | 2047 | CSSY |
| type99 | | FDD | | GS | | GDTA | | SDVG | | NRP | | TSSS |
| | | Y | | | | MVTV | | GYNY | | S | | TLVF |
| | | | | | | FDYW | | VS | | | | |
| clono- | 1108 | GYS | 1296 | YPSD | 1484 | CARQ | 1672 | TGTS | 1860 | DVS | 2048 | CCSY |
| type102 | | FTS | | SD | | INWG | | SDVG | | KRP | | AGSS |
| | | Y | | | | AIDY | | GYNY | | S | | TFVV |
| | | | | | | W | | VS | | | | F |
| clono- | 1109 | GYT | 1297 | SVYN | 1485 | CARQ | 1673 | TGTS | 1861 | DVS | 2049 | CCSY |
| type103 | | FTS | | GN | | ITAT | | SDVG | | KRP | | AGSS |
| | | Y | | | | RGFD | | GYNY | | S | | TFVV |
| | | | | | | YW | | VS | | | | F |
| clono- | 1110 | GDS | 1298 | YYRS | 1486 | CARK | 1674 | TGTS | 1862 | DVS | 2050 | CCSY |
| type109 | | VSS | | KWY | | WELR | | SDVG | | KRP | | AGSS |
| | | NSA | | | | DTFD | | GYNY | | S | | TLGI |
| | | | | | | IW | | VS | | | | DWVF |
| clono- | 1111 | GGS | 1299 | NHSG | 1487 | CASY | 1675 | TLSS | 1863 | VKS | 2051 | CGES |
| type110 | | FSG | | S | | GDFF | | EHST | | DGS | | HTID |
| | | Y | | | | DYW | | YTIE | | HSK | | GQVG |
| | | | | | | | | | | GD | | VVF |
| clono- | 1112 | GFT | 1300 | SSSS | 1488 | CARE | 1676 | TLSS | 1864 | VKS | 2052 | CGES |
| type111 | | FSS | | SY | | TELT | | EHST | | DGS | | HTID |
| | | Y | | | | VMDV | | YTIE | | HSK | | GQVG |
| | | | | | | W | | | | GD | | WVF |
| clono- | 1113 | GFT | 1301 | KSKT | 1489 | CTTD | 1677 | QGDS | 1865 | GKN | 2053 | CNSR |
| type112 | | FSN | | DGGT | | WEYY | | LRSY | | NRP | | DSSG |
| | | A | | | | DFWS | | YAS | | S | | NHVV |
| | | | | | | GYYS | | | | | | F |
| | | | | | | PYFD | | | | | | |
| | | | | | | YW | | | | | | |
| clono- | 1114 | GGS | 1302 | YYIG | 1490 | CARA | 1678 | GGNN | 1866 | YDT | 2054 | CQVW |
| type397 | | ISS | | I | | FDYW | | VGSK | | DRP | | DSRS |
| | | GGY | | | | | | SVH | | S | | DHVV |
| | | | | | | | | | | | | F |
| clono- | 1115 | GGS | 1303 | YYSG | 1491 | CVRA | 1679 | GGNT | 1867 | YDS | 2055 | CQVW |
| type398 | | ISS | | S | | FDYW | | FGSK | | DRP | | DLYS |
| | | GGY | | | | | | TVH | | S | | AHVV |
| | | | | | | | | | | | | F |
| clono- | 1116 | GFT | 1304 | KSKT | 1492 | CTTG | 1680 | SGDA | 1868 | EDS | 2056 | CYST |
| type399 | | FSN | | DGGT | | ANW | | LPKK | | KRP | | DSSG |
| | | A | | | | | | YAY | | S | | NHWV |
| | | | | | | | | | | | | F |
| clono- | 1117 | GYT | 1305 | NPNS | 1493 | CARV | 1681 | TGTS | 1869 | EVS | 2057 | CSSY |
| type400 | | FTS | | GN | | AFDI | | SDVG | | NRP | | TSSS |
| | | Y | | | | W | | GYNY | | S | | TVF |
| | | | | | | | | VS | | | | |
| clono- | 1118 | GYT | 1306 | SAYN | 1494 | CARQ | 1682 | TGTS | 1870 | EVI | 2058 | CSAY |
| type401 | | FTT | | GN | | IGDY | | SDVG | | KRP | | AGSN |
| | | Y | | | | W | | GYNY | | S | | NVVF |
| | | | | | | | | VS | | | | |
| clono- | 1119 | GFT | 1307 | SGSG | 1495 | CH?T | 1683 | SGSS | 1871 | SNN | 2059 | CAAW |
| type402 | | FSS | | GS | | GEDY | | SNIG | | QRP | | DDSL |
| | | Y | | | | W | | SNTV | | S | | NGWV |
| | | | | | | | | N | | | | F |
| clono- | 1120 | GFT | 1308 | KSKS | 1496 | CTTG | 1684 | SADA | 1872 | KDS | 2060 | COSA |
| type407 | | FSN | | DGGT | | GTHW | | LPKQ | | ERP | | DSSA |
| | | A | | | | | | YAY | | S | | TWVF |
| clono- | 1121 | GYT | 1309 | NPNS | 1497 | CARR | 1685 | SGDA | 1873 | EDS | 2061 | CYST |
| type408 | | FTS | | GN | | SFLD | | LPKK | | KRP | | DSSG |
| | | Y | | | | YW | | YAY | | S | | NHRV |
| | | | | | | | | | | | | F |
| clono- | 1122 | GFT | 1310 | KSKT | 1498 | CTTG | 1686 | SADA | 1874 | KDS | 2062 | CQSL |
| type409 | | FSN | | DGGT | | GTNW | | LPKQ | | ERP | | DSSG |
| | | A | | | | | | YAY | | S | | TYWV |
| | | | | | | | | | | | | F |
| clono- | 1123 | GFT | 1311 | SSSS | 1499 | CARE | 1687 | SGDK | 1875 | QDS | 2063 | CQAW |
| type413 | | FSS | | SY | | SSGF | | LGDK | | KRP | | DSST |
| | | Y | | | | DYW | | YAC | | S | | VVF |
| clono- | 1124 | GYT | 1312 | NPNS | 1500 | CARG | 1688 | SGDK | 1876 | QDS | 2064 | CQAW |
| type414 | | FTS | | GN | | SSWF | | LGDK | | KRP | | DSST |
| | | Y | | | | DYW | | YAC | | S | | VVF |
| clono- | 1125 | GFT | 1313 | KSKT | 1501 | CTLN | 1689 | SGDK | 1877 | QDS | 2065 | CQAW |
| type415 | | FSN | | DGGT | | WGDY | | LGDK | | KRP | | DSST |
| | | A | | | | W | | YAC | | S | | VVF |
| clono- | 1126 | GFT | 1314 | SSSS | 1502 | CARA | 1690 | QGDS | 1878 | GKN | 2066 | CNSR |
| type418 | | FSS | | SY | | ADAF | | LRSY | | NRP | | DSSG |
| | | Y | | | | DIW | | YAS | | S | | NHWV |
| | | | | | | | | | | | | F |
| clono- | 1127 | GFT | 1315 | GTAG | 1503 | CARG | 1691 | TGTS | 1879 | DVS | 2067 | CCSY |
| type419 | | FSS | | D | | GSDA | | SDVG | | KRP | | AGSV |
| | | Y | | | | FDIW | | GYNY | | S | | VF |
| | | | | | | | | VS | | | | |
| clono- | 1128 | EFT | 1316 | RSEI | 1504 | CTTD | 1692 | QGDS | 1880 | GKN | 2068 | CNSR |
| type420 | | FRN | | DGGT | | HPYY | | LRSY | | NRP | | DSSG |
| | | A | | | | W | | YAS | | S | | NHVV |
| | | | | | | | | | | | | F |
| clono- | 1129 | GFT | 1317 | KSKT | 1505 | CTTD | 1693 | QGDS | 1881 | GKN | 2069 | CNSR |
| type421 | | FSN | | DGGT | | HPYY | | LRSY | | NRP | | DSSG |
| | | A | | | | W | | YAS | | S | | NHWV |
| | | | | | | | | | | | | F |
| clono- | 1130 | GFT | 1318 | WYDG | 1506 | CALA | 1694 | SGSS | 1882 | YDD | 2070 | CAAW |
| type423 | | FSN | | SN | | VTGF | | SNIG | | LLP | | DDRL |
| | | Y | | | | DYW | | NNAV | | S | | NGPV |
| | | | | | | | | N | | | | F |
| clono- | 1131 | GFT | 1319 | SSSG | 1507 | CARD | 1695 | TGTS | 1883 | DVS | 2071 | CCSY |
| type424 | | FSD | | ST | | GAAF | | SDVG | | KRP | | AGSS |
| | | Y | | | | DIW | | GYNY | | S | | TLVF |
| | | | | | | | | VS | | | | |
| clono- | 1132 | GYT | 1320 | NTYN | 1508 | CARD | 1696 | SGDK | 1884 | QDS | 2072 | CQAW |
| type426 | | FTN | | DK | | RGYS | | LGDK | | KRP | | DSST |
| | | Y | | | | FDYW | | HAC | | S | | F |
| clono- | 1133 | GYT | 1321 | SAYN | 1509 | CARN | 1697 | SGDV | 1885 | KDS | 2073 | CYSA |
| type427 | | FTS | | GN | | HYYY | | LAKK | | ERP | | ADNN |
| | | Y | | | | LDVW | | YAR | | S | | RVF |
| clono- | 1134 | GFT | 1322 | GTAG | 1510 | CARV | 1698 | SGDV | 1886 | KDS | 2074 | CYSA |
| type428 | | FSS | | D | | SPTG | | LAKK | | ERP | | ADNN |
| | | Y | | | | TTDY | | YAR | | S | | LVF |
| | | | | | | W | | | | | | |
| clono- | 1135 | GFT | 1323 | KSKT | 1511 | CTAR | 1699 | SGDV | 1887 | KDS | 2075 | CYSA |
| type429 | | FSN | | DGGT | | PLGD | | LAKK | | ERP | | ADNN |
| | | A | | | | VW | | YAR | | S | | YVF |
| clono- | 1136 | GFT | 1324 | KSKT | 1512 | CTTD | 1700 | SGDV | 1888 | KDS | 2076 | CYSA |
| type430 | | FSN | | DGGT | | NGFD | | LAKK | | ERP | | ADNN |
| | | A | | | | YW | | YAR | | S | | LVF |
| clono- | 1137 | GFT | 1325 | SSSS | 1513 | CARD | 1701 | QGDR | 1889 | GKN | 2077 | CNSR |
| type431 | | FSS | | SY | | LISS | | LRSY | | NRP | | DSSG |
| | | Y | | | | FDYW | | YAS | | S | | NHLV |
| | | | | | | | | | | | | F |
| clono- | 1138 | GFT | 1326 | KQDG | 1514 | CARR | 1702 | QGDS | 1890 | GKN | 2078 | CNSR |
| type432 | | FSS | | SE | | IVGA | | LRSY | | NRP | | DSSG |
| | | Y | | | | FDYW | | YAS | | S | | NHLV |
| | | | | | | | | | | | | F |
| clono- | 1139 | RYT | 1327 | NPSG | 1515 | CARG | 1703 | GGNN | 1891 | YDS | 2079 | CQVW |
| type434 | | FTS | | GT | | GWGT | | IGSK | | DRP | | DSSS |
| | | Y | | | | MDVW | | SVH | | S | | DHVV |
| | | | | | | | | | | | | F |
| clono- | 1140 | GFT | 1328 | SSSS | 1516 | CARE | 1704 | SGDA | 1892 | EDS | 2080 | CYST |
| type435 | | FSS | | ST | | GWEL | | LPKK | | KRP | | DSSG |
| | | Y | | | | LDYW | | YAY | | S | | NHRV |
| | | | | | | | | | | | | F |
| clono- | 1141 | GLT | 1329 | YSGG | 1517 | CARD | 1705 | ASST | 1893 | STS | 2081 | CLLY |
| type436 | | VST | | G | | NWDS | | GAVT | | NKH | | YGGA |
| | | N | | | | YFDY | | SGYY | | S | | RVF |
| | | | | | | W | | PN | | | | |
| clono- | 1142 | GFT | 1330 | NWNG | 1518 | CART | 1706 | TGTS | 1894 | EVS | 2082 | CSSY |
| type437 | | FDD | | GS | | TVTH | | SDVG | | KRP | | AGSN |
| | | Y | | | | MDVW | | GYNY | | S | | NLVF |
| | | | | | | | | VS | | | | |
| clono- | 1143 | GFT | 1331 | YSGG | 1519 | CARD | 1707 | TGTS | 1895 | DVS | 2083 | CCSY |
| type438 | | VSS | | S | | WNYD | | SDVG | | KRP | | AGSS |
| | | N | | | | AFDI | | GYNY | | S | | TWVF |
| | | | | | | W | | VS | | | | |
| clono- | 1144 | GFT | 1332 | SSSS | 1520 | CARG | 1708 | TGTS | 1896 | DVS | 2084 | CCSY |
| type439 | | FSS | | SY | | DPGW | | SDVG | | KRP | | AGSS |
| | | Y | | | | FDPW | | GYNY | | S | | TFWV |
| | | | | | | | | VS | | | | F |
| clono- | 1145 | GFT | 1333 | NSDG | 1521 | CARE | 1709 | SGDK | 1897 | QDS | 2085 | CQAW |
| type442 | | FSS | | SS | | NWNY | | LGDK | | KRP | | DSST |
| | | Y | | | | WFDP | | YAC | | S | | VVF |
| | | | | | | W | | | | | | |
| clono- | 1146 | GGS | 1334 | YHSG | 1522 | CARL | 1710 | SGDK | 1898 | QDN | 2086 | CQAW |
| type443 | | ISS | | S | | RPGD | | LGDK | | KRP | | DSST |
| | | NN | | | | SFDY | | YAC | | S | | ALVF |
| | | | | | | W | | | | | | |
| clono- | 1147 | GYT | 1335 | NTNI | 1523 | CARS | 1711 | SGDK | 1899 | QDS | 2087 | CQAW |
| type444 | | FTR | | GN | | PNWG | | LGDK | | KRP | | DSST |
| | | N | | | | LFDY | | YAC | | S | | SGVF |
| | | | | | | W | | | | | | |
| clono- | 1148 | GFT | 1336 | SSSS | 1524 | CARD | 1712 | QGDS | 1900 | GKN | 2088 | CNSR |
| type445 | | FSS | | SY | | RGAT | | LRSY | | NRP | | DSSG |
| | | Y | | | | GFDY | | YAS | | S | | NHWV |
| | | | | | | W | | | | | | F |
| clono- | 1149 | GYT | 1337 | SAYN | 1525 | CARE | 1713 | SGDA | 1901 | EDS | 2089 | CYST |
| type446 | | FTS | | GN | | SGEL | | LPKK | | KRP | | DSSG |
| | | Y | | | | LGDY | | YAY | | S | | NHRV |
| | | | | | | W | | | | | | F |
| clono- | 1150 | GFT | 1338 | GTAG | 1526 | CARY | 1714 | QGDS | 1902 | GKN | 2090 | CNSR |
| type448 | | FSS | | D | | SGSY | | LRSY | | NRP | | DSSG |
| | | Y | | | | YYFD | | YAS | | S | | NHVV |
| | | | | | | YW | | | | | | F |
| clono- | 1151 | GFT | 1339 | WYDG | 1527 | CARG | 1715 | SGDA | 1903 | EDS | 2091 | CYST |
| type450 | | FSS | | SN | | IAAA | | LPKK | | KRP | | DSSG |
| | | Y | | | | GKDY | | YAY | | S | | NHAV |
| | | | | | | W | | | | | | F |
| clono- | 1152 | GYS | 1340 | YPGD | 1528 | CARQ | 1716 | GGNN | 1904 | YDS | 2092 | CQVW |
| type451 | | FTS | | SD | | DSNY | | IGSK | | DRP | | DSSS |
| | | Y | | | | VFDY | | SVH | | S | | DHVV |
| | | | | | | W | | | | | | F |
| clono- | 1153 | GFT | 1341 | KYDG | 1529 | CARD | 1717 | SGSI | 1905 | SNN | 2093 | CATW |
| type452 | | FSN | | RE | | HSAW | | SNLG | | QRP | | DDSL |
| | | Y | | | | SFDY | | SNTV | | S | | NGRV |
| | | | | | | W | | N | | | | F |
| clono- | 1154 | GFT | 1342 | KQDG | 1530 | CARR | 1718 | TGTS | 1906 | EVS | 2094 | CSSY |
| type453 | | FSS | | SE | | RGSC | | SDVG | | KRP | | AGSN |
| | | Y | | | | SFDY | | GYNY | | S | | NLVF |
| | | | | | | W | | VS | | | | |
| clono- | 1155 | GYT | 1343 | SAYN | 1531 | CARR | 1719 | TGTS | 1907 | EVS | 2095 | CSSY |
| type454 | | FTS | | GN | | SYAN | | SDVG | | KRP | | AGSN |
| | | Y | | | | CFDY | | GYNY | | S | | NWVF |
| | | | | | | W | | VS | | | | |
| clono- | 1156 | GFT | 1344 | NSDG | 1532 | CARD | 1720 | TGTS | 1908 | DVS | 2096 | CCSY |
| type455 | | FSS | | SS | | EQLV | | SDVG | | KRP | | AGSS |
| | | Y | | | | PFDI | | GYNY | | S | | TLVF |
| | | | | | | W | | VS | | | | |
| clono- | 1157 | GFT | 1345 | YSGG | 1533 | CARD | 1721 | TGTS | 1909 | DVS | 2097 | CCSY |
| type456 | | VSS | | S | | GAAA | | SDVG | | KRP | | AGSS |
| | | N | | | | GDFQ | | GYNY | | S | | TWVF |
| | | | | | | HW | | VS | | | | |
| clono- | 1158 | GFT | 1346 | SWNS | 1534 | CAKD | 1722 | TGTS | 1910 | EVS | 2098 | CSSY |
| type457 | | FDD | | GS | | SGSY | | SDVG | | KRP | | AGSN |
| | | Y | | | | YFDY | | GYNY | | S | | NFVV |
| | | | | | | W | | VS | | | | F |
| clono- | 1159 | GFT | 1347 | SSSG | 1535 | CARD | 1723 | TGSS | 1911 | GNS | 2099 | CQSY |
| type458 | | FSD | | ST | | GQLW | | SNIG | | NRP | | DSSL |
| | | Y | | | | SFDY | | AGYD | | S | | SDVV |
| | | | | | | W | | VH | | | | F |
| clono- | 1160 | GFT | 1348 | YAGG | 1536 | CGRV | 1724 | TGSS | 1912 | GNN | 2100 | CQSY |
| type459 | | VSR | | N | | VPIG | | SNIG | | NRP | | DSSL |
| | | N | | | | NWED | | AGYD | | S | | SGWV |
| | | | | | | PW | | VH | | | | F |
| clono- | 1161 | GFT | 1349 | KQDG | 1537 | CARD | 1725 | TLRS | 1913 | YKS | 2101 | CMIW |
| type460 | | FSS | | SE | | SNWG | | GINV | | DSD | | HSSA |
| | | Y | | | | VFDY | | GTYR | | KQQ | | WVF |
| | | | | | | W | | IY | | GS | | |
| clono- | 1162 | GFT | 1350 | KQDG | 1538 | CARD | 1726 | TLRS | 1914 | YKS | 2102 | CMIW |
| type461 | | FSS | | SE | | RLTG | | GINV | | DSD | | HSSA |
| | | Y | | | | DLDY | | GTYR | | KQQ | | WVF |
| | | | | | | W | | IY | | GS | | |
| clono- | 1163 | GFT | 1351 | NIVG | 1539 | CARE | 1727 | RGNN | 1915 | RNI | 2103 | CQVW |
| type464 | | FSR | | ST | | GDRS | | IGSQ | | NRP | | DSSG |
| | | Y | | | | DAFA | | NVH | | S | | SWVF |
| | | | | | | IW | | | | | | |
| clono- | 1164 | GFT | 1352 | SSSS | 1540 | CARQ | 1728 | SGDA | 1916 | EDS | 2104 | CYST |
| type465 | | FSS | | SY | | QWLG | | LPKK | | KRP | | DSSG |
| | | Y | | | | YYFD | | YAY | | S | | NHRV |
| | | | | | | YW | | | | | | F |
| clono- | 1165 | GFT | 1353 | KEDG | 1541 | CARD | 1729 | QGDS | 1917 | GKS | 2105 | CNSR |
| type466 | | FSI | | SE | | SNFL | | LRSF | | NRP | | DTSG |
| | | Y | | | | YYFD | | YAS | | S | | NYLV |
| | | | | | | YW | | | | | | F |
| clono- | 1166 | GYT | 1354 | SAYN | 1542 | CARQ | 1730 | QGDS | 1918 | GKN | 2106 | CNSR |
| type468 | | FTS | | GN | | ITGT | | LRSY | | NRP | | DSSG |
| | | Y | | | | RGFD | | YAS | | S | | NHWV |
| | | | | | | YW | | | | | | F |
| clono- | 1167 | GYT | 1355 | NPNS | 1543 | CARM | 1731 | SGDA | 1919 | EDS | 2107 | CYST |
| type469 | | FTS | | GN | | GYSN | | LPKK | | KRP | | DSSG |
| | | Y | | | | YPFD | | YAY | | S | | NHVV |
| | | | | | | YW | | | | | | F |
| clono- | 1168 | GYT | 1356 | NPSG | 1544 | CARG | 1732 | QGDS | 1920 | GKN | 2108 | CNSR |
| type470 | | FTS | | GS | | IPTT | | LRSY | | NRP | | DSSG |
| | | Y | | | | VTPD | | YAS | | S | | NHLV |
| | | | | | | YW | | | | | | F |
| clono- | 1169 | GFT | 1357 | GTAG | 1545 | CARA | 1733 | SGDA | 1921 | EDS | 2109 | CYST |
| type471 | | FSS | | D | | GLLT | | LPKK | | KRP | | DSSG |
| | | Y | | | | GDAF | | YAY | | S | | NHRV |
| | | | | | | DIW | | | | | | F |
| clono- | 1170 | GFT | 1358 | KSKT | 1546 | CITG | 1734 | ASST | 1922 | STS | 2110 | CLLY |
| type474 | | FSN | | DGGT | | TTFP | | GAVT | | NKH | | YGGA |
| | | A | | | | FDYW | | SGYY | | S | | WVF |
| | | | | | | | | PN | | | | |
| clono- | 1171 | GFT | 1359 | SSNG | 1547 | CTKG | 1735 | TGTS | 1923 | EVS | 2111 | CSSY |
| type475 | | FSS | | GN | | GVGA | | SDVG | | NRP | | TSSS |
| | | H | | | | SFDY | | GYNY | | S | | TWVF |
| | | | | | | W | | VS | | | | |
| clono- | 1172 | GFT | 1360 | SSSS | 1548 | CARG | 1736 | SGSS | 1924 | SNN | 2112 | CAAW |
| type476 | | FSS | | SY | | DYSN | | SNIG | | QRP | | DDSL |
| | | Y | | | | YYFD | | SNTV | | S | | NGWV |
| | | | | | | YW | | N | | | | F |
| clono- | 1173 | GGS | 1361 | NHSG | 1549 | CARW | 1737 | TGTS | 1925 | EVS | 2113 | CSSY |
| type477 | | FSG | | S | | EQPW | | SDVG | | KRP | | AGSN |
| | | Y | | | | | | GYNY | | S | | NWVF |
| | | | | | | | | VS | | | | |
| clono- | 1174 | GYT | 1362 | NPSG | 1550 | CARR | 1738 | TGTS | 1926 | DVS | 2114 | CCSY |
| type478 | | FTS | | GS | | TGTT | | SDVG | | KRP | | AGSS |
| | | Y | | | | HYFD | | GYNY | | S | | TLVF |
| | | | | | | YW | | VS | | | | |
| clono- | 1175 | GFT | 1363 | SSSG | 1551 | CARG | 1739 | TGTS | 1927 | DVS | 2115 | CCSY |
| type479 | | FSD | | ST | | LWLG | | SDVG | | KRP | | AGSS |
| | | Y | | | | LYFD | | GYNY | | S | | TWVF |
| | | | | | | YW | | VS | | | | |
| clono- | 1176 | GYS | 1364 | YPGD | 1552 | CARF | 1740 | TGTS | 1928 | EVS | 2116 | CSSY |
| type480 | | FTS | | SD | | LGSS | | SDVG | | KRP | | AGSN |
| | | Y | | | | YYFD | | GYNY | | S | | NFEV |
| | | | | | | YW | | VS | | | | F |
| clono- | 1177 | GFT | 1365 | SSSS | 1553 | CARG | 1741 | TLRS | 1929 | YKS | 2117 | CMIW |
| type481 | | FSS | | ST | | GAAA | | GINV | | DSD | | HSSA |
| | | Y | | | | GAFD | | GTYR | | KQQ | | WVF |
| | | | | | | IW | | IY | | GS | | |
| clono- | 1178 | GGS | 1366 | FYSG | 1554 | CARA | 1742 | SGDK | 1930 | QDS | 2118 | CQAW |
| type486 | | ISS | | S | | EWEL | | LGDK | | KRP | | DSST |
| | | GGY | | | | LWFD | | YAC | | S | | VVF |
| | | | | | | PW | | | | | | |
| clono- | 1179 | GFS | 1367 | YWND | 1555 | CAHN | 1743 | SADA | 1931 | KDS | 2119 | CQSA |
| type487 | | LST | | D | | YFYI | | LPKQ | | ERP | | NSGT |
| | | SGV | | | | SGYF | | YAY | | S | | WVF |
| | | | | | | YW | | | | | | |
| clono- | 1180 | GFT | 1368 | SYDG | 1556 | CAKD | 1744 | SGDK | 1932 | QDT | 2120 | CQAW |
| type488 | | FSS | | SN | | PLRV | | LGDK | | KRP | | DSST |
| | | Y | | | | VNYM | | YAC | | S | | VVF |
| | | | | | | DVW | | | | | | |
| clono- | 1181 | GFS | 1369 | YWSD | 1557 | CAQT | 1745 | SADA | 1933 | KDS | 2121 | CQSA |
| type490 | | LST | | D | | GYNS | | LPNQ | | ERP | | DSSG |
| | | SGV | | | | WSFD | | YAY | | S | | TWVF |
| | | | | | | YW | | | | | | |
| clono- | 1182 | GYP | 1370 | SAYN | 1558 | CARE | 1746 | QGDS | 1934 | GKN | 2122 | CNSR |
| type492 | | FTS | | SN | | DAWN | | LRSY | | NRP | | DSSG |
| | | Y | | | | YGWF | | YAS | | S | | NHVV |
| | | | | | | DPW | | | | | | F |
| clono- | 1183 | GYS | 1371 | SAYN | 1559 | CARE | 1747 | SGDA | 1935 | EDS | 2123 | CYST |
| type493 | | FTG | | GN | | ILWL | | LPKK | | KRP | | DSSG |
| | | Y | | | | GGYF | | YAY | | S | | NHRV |
| | | | | | | DYW | | | | | | F |
| clono- | 1184 | GYT | 1372 | NTNT | 1560 | CARE | 1748 | QGDS | 1936 | GKN | 2124 | CNSR |
| type494 | | FSS | | GN | | YSSG | | LRSY | | NRP | | DSSG |
| | | Y | | | | WYYF | | YAS | | S | | NHLV |
| | | | | | | DYW | | | | | | F |
| clono- | 1185 | GYT | 1373 | NPNS | 1561 | CARE | 1749 | SGDA | 1937 | EDS | 2125 | CYST |
| type495 | | FTG | | GG | | RIAV | | LPKK | | KRP | | DSSG |
| | | Y | | | | APPF | | YAY | | S | | NHRV |
| | | | | | | DYW | | | | | | F |
| clono- | 1186 | GYT | 1374 | NPNS | 1562 | CARA | 1750 | SGDA | 1938 | EDS | 2126 | CYST |
| type496 | | FTS | | GN | | GWEL | | LPKK | | KRP | | DSSG |
| | | Y | | | | PEYF | | YAY | | S | | NHRV |
| | | | | | | QHW | | | | | | F |
| clono- | 1187 | GYT | 1375 | NPNS | 1563 | CARG | 1751 | SGDA | 1939 | EDS | 2127 | CYST |
| type497 | | FTS | | GN | | GDDY | | LPKK | | KRP | | DSSG |
| | | Y | | | | SNLF | | YAY | | S | | NHRV |
| | | | | | | DYW | | | | | | F |
| clono- | 1188 | GDT | 1376 | IPIF | 1564 | CART | 1752 | GGNN | 1940 | YDS | 2128 | CQVW |
| type498 | | FSN | | AT | | PLGI | | IGSK | | DRP | | DSNS |
| | | F | | | | GRSF | | SVH | | S | | DHWV |
| | | | | | | DLW | | | | | | F |
| clono- | 1189 | GFT | 1377 | KRKT | 1565 | CTTA | 1753 | SADA | 1941 | KDS | 2129 | COSA |
| type499 | | FSN | | DGGT | | STVT | | LPKQ | | ERP | | DSSG |
| | | A | | | | TGDY | | YAY | | S | | TYPV |
| | | | | | | W | | | | | | F |
| clono- | 1190 | GDS | 1378 | YYRS | 1566 | CARE | 1754 | QGDS | 1942 | GKN | 2130 | CNSR |
| type501 | | VSS | | KWY | | RTEI | | LRSY | | NRP | | DSSG |
| | | NSA | | | | DYW | | YAS | | S | | NHWV |
| | | | | | | | | | | | | F |
| clono- | 1191 | GFT | 1379 | KSKT | 1567 | CTTG | 1755 | GSST | 1943 | DTS | 2131 | CLLS |
| type502 | | FNN | | DGGT | | RYFD | | GAVT | | NKH | | YSGA |
| | | A | | | | WFDY | | SGHY | | S | | RVF |
| | | | | | | W | | PY | | | | |
| clono- | 1192 | GFT | 1380 | KSKT | 1568 | CTTA | 1756 | TGTS | 1944 | EVS | 2132 | CSSY |
| type504 | | FSN | | DGGT | | SGSY | | SDVG | | KRP | | AGSN |
| | | A | | | | WFDP | | GYNY | | S | | NLVE |
| | | | | | | W | | VS | | | | |
| clono- | 1193 | GFT | 1381 | SYDG | 1569 | CAKG | 1757 | TGTS | 1945 | DVS | 2133 | CCSY |
| type505 | | FSS | | SN | | NWNY | | SDVG | | KRP | | AGSS |
| | | Y | | | | GDAF | | GYNY | | S | | TYVF |
| | | | | | | DIW | | VS | | | | |
| clono- | 1194 | GFT | 1382 | NWNG | 1570 | CARE | 1758 | TGTS | 1946 | EVS | 2134 | CSSY |
| type506 | | FDD | | GS | | NYDF | | SDVG | | NRP | | TSSS |
| | | Y | | | | WSGF | | GYNY | | S | | TVVF |
| | | | | | | DPW | | VS | | | | |
| clono- | 1195 | GDS | 1383 | YYRS | 1571 | CARE | 1759 | TGTS | 1947 | DVS | 2135 | CCSY |
| type507 | | VSS | | KWY | | DRGF | | SDVG | | KRP | | AGSS |
| | | NSA | | | | DYW | | GYNY | | S | | NVVF |
| | | | | | | | | VS | | | | |
| clono- | 1196 | GFT | 1384 | SWNS | 1572 | CAKR | 1760 | TGTS | 1948 | DVS | 2136 | CCSY |
| type508 | | FDD | | GS | | AVVT | | SDVG | | KRP | | AGSS |
| | | Y | | | | DYYM | | GYNY | | S | | TFWV |
| | | | | | | DVW | | VS | | | | F |
| clono- | 1197 | GFT | 1385 | SSSS | 1573 | CART | 1761 | TGTS | 1949 | EVS | 2137 | CSSY |
| type509 | | FSS | | NT | | SSWS | | SDVG | | KRP | | AGSN |
| | | Y | | | | YDAF | | GYNY | | S | | NFVV |
| | | | | | | DIW | | VS | | | | F |
| clono- | 1198 | GYT | 1386 | NPSG | 1574 | CARE | 1762 | SGDK | 1950 | QDS | 2138 | CQAT |
| type511 | | FTS | | GS | | RGHT | | LGDK | | KRP | | EVE |
| | | Y | | | | VTPY | | YAC | | S | | |
| | | | | | | FDYW | | | | | | |
| clono- | 1199 | GEN | 1387 | SNTG | 1575 | CARD | 1763 | SGDV | 1951 | KDS | 2139 | CYSA |
| type512 | | FSS | | NT | | GPQV | | LAKK | | ERP | | ADNK |
| | | Y | | | | GATD | | YAR | | S | | VF |
| | | | | | | FDYW | | | | | | |
| clono- | 1200 | GFT | 1388 | KRKT | 1576 | CTTE | 1764 | SGDK | 1952 | QDS | 2140 | CQAW |
| type513 | | FSN | | DGGT | | YSSS | | LGDK | | KRP | | DSST |
| | | A | | | | ENFD | | YAC | | S | | AVF |
| | | | | | | YW | | | | | | |
| clono- | 1201 | GFT | 1389 | NSDG | 1577 | CARD | 1765 | SGDK | 1953 | QDS | 2141 | CQAW |
| type514 | | FSS | | SS | | LGAA | | LGDK | | KRP | | DSST |
| | | Y | | | | RPRG | | YAC | | S | | VVF |
| | | | | | | FDYW | | | | | | |
| clono- | 1202 | GFT | 1390 | SGSG | 1578 | CAKE | 1766 | SGDA | 1954 | EDS | 2142 | CYST |
| type515 | | FSS | | GS | | GDSG | | LPKK | | KRP | | DSSG |
| | | Y | | | | YDSA | | YAY | | S | | NRVF |
| | | | | | | FDIW | | | | | | |
| clono- | 1203 | GGS | 1391 | YHSG | 1579 | CARV | 1767 | SGDA | 1955 | EDS | 2143 | CYST |
| type517 | | ISS | | S | | LNWN | | LPKK | | KRP | | DSSG |
| | | NN | | | | YGDA | | YAY | | S | | NHRG |
| | | | | | | FDIW | | | | | | F |
| clono- | 1204 | GGS | 1392 | YHSG | 1580 | CARD | 1768 | TGTS | 1956 | DVS | 2144 | CCSY |
| type518 | | ISS | | S | | PSIV | | SDVG | | KRP | | AQGV |
| | | SN | | | | GATA | | GYNY | | S | | VF |
| | | | | | | FDIW | | VS | | | | |
| clono- | 1205 | GGS | 1393 | YHSG | 1581 | CARS | 1769 | QGDS | 1957 | GKN | 2145 | CNSR |
| type519 | | IIS | | S | | HIVG | | LRSY | | NRP | | DSSG |
| | | SN | | | | VNGG | | YAS | | S | | NHWV |
| | | | | | | FDYW | | | | | | F |
| clono- | 1206 | GFT | 1394 | SSSS | 1582 | CARD | 1770 | QGDS | 1958 | GKN | 2146 | CNSR |
| type520 | | FSS | | SY | | RYNW | | LRSY | | NRP | | DSSG |
| | | Y | | | | NYRA | | YAS | | S | | NHLV |
| | | | | | | FDIW | | | | | | F |
| clono- | 1207 | GFT | 1395 | KQDG | 1583 | CARD | 1771 | QGDS | 1959 | GKN | 2147 | CNSR |
| type522 | | FSS | | SE | | LGRG | | LRSY | | NRP | | DSSG |
| | | Y | | | | TISW | | YAS | | S | | NHWV |
| | | | | | | FDPW | | | | | | F |
| clono- | 1208 | GFS | 1396 | YWNG | 1584 | CTQT | 1772 | GGNN | 1960 | YDS | 2148 | CQVW |
| type523 | | LNS | | D | | GYDS | | IGSK | | DRP | | DSSS |
| | | SGV | | | | RWSF | | SVH | | S | | DHWV |
| | | | | | | AYW | | | | | | F |
| clono- | 1209 | GYI | 1397 | SAYN | 1585 | CARE | 1773 | QGDS | 1961 | GKN | 2149 | CNSR |
| type524 | | FMN | | GN | | GQWR | | LRSY | | NRP | | DSSG |
| | | Y | | | | GRGW | | YAS | | S | | NHLV |
| | | | | | | FALW | | | | | | F |
| clono- | 1210 | GYT | 1398 | NTNT | 1586 | CARE | 1774 | QGDS | 1962 | GKN | 2150 | CNSR |
| type526 | | FTN | | GK | | RYFE | | LRSY | | NRP | | DSSG |
| | | Y | | | | DFHY | | YAS | | S | | NHLV |
| | | | | | | MDVW | | | | | | F |
| clono- | 1211 | GYT | 1399 | NPNS | 1587 | CARS | 1775 | QGDS | 1963 | GKN | 2151 | CNSR |
| type527 | | FTD | | GG | | SWLQ | | LRSY | | NRP | | DSSG |
| | | N | | | | LTYY | | YAS | | S | | NHLL |
| | | | | | | FDYW | | | | | | F |
| clono- | 1212 | GYT | 1400 | NPSG | 1588 | CARE | 1776 | QGDS | 1964 | GKK | 2152 | CKSR |
| type528 | | FTS | | GS | | GLQL | | LRSY | | NRP | | DSSG |
| | | Y | | | | GSNW | | YAS | | S | | NHVV |
| | | | | | | FDPW | | | | | | F |
| clono- | 1213 | GFT | 1401 | SSSS | 1589 | CARN | 1777 | SGDA | 1965 | EDS | 2153 | CYST |
| type529 | | FSS | | ST | | DILT | | LPKK | | KRP | | DSSG |
| | | Y | | | | GEDA | | YAY | | S | | NHRV |
| | | | | | | FDIW | | | | | | F |
| clono- | 1214 | GFT | 1402 | SGSG | 1590 | CAKE | 1778 | QGDS | 1966 | GKN | 2154 | CNSR |
| type530 | | FSN | | GR | | SIIV | | FRNY | | NRP | | DSSG |
| | | Y | | | | GATM | | YAS | | S | | NHWV |
| | | | | | | FDYW | | | | | | F |
| clono- | 1215 | GFT | 1403 | SYDG | 1591 | CAKG | 1779 | QGDS | 1967 | GKN | 2155 | CNSR |
| type531 | | FSS | | SN | | IAAL | | LRSY | | NRP | | DSSG |
| | | Y | | | | GYYY | | YAS | | S | | NHLV |
| | | | | | | MDVW | | | | | | F |
| clono- | 1216 | GYR | 1404 | YPGD | 1592 | CAKR | 1780 | QGDS | 1968 | GKN | 2156 | CNSR |
| type532 | | FSN | | SD | | RITG | | LRSY | | NRP | | DSSG |
| | | Y | | | | SHNW | | YAS | | S | | NHLV |
| | | | | | | FDPW | | | | | | F |
| clono- | 1217 | GYT | 1405 | NTNT | 1593 | CARG | 1781 | ASST | 1969 | STS | 2157 | CLLY |
| type534 | | FTS | | GN | | GTIF | | GAVT | | NKH | | YGGA |
| | | Y | | | | GVVN | | SGYY | | S | | RVF |
| | | | | | | FDYW | | PN | | | | |
| clono- | 1218 | GFT | 1406 | WYDG | 1594 | CLSR | 1782 | TGTS | 1970 | DVT | 2158 | CCSY |
| type537 | | FSS | | SN | | SGYS | | SDVG | | KRP | | AGSS |
| | | Y | | | | AHND | | VYNF | | S | | TWVF |
| | | | | | | GDYW | | VS | | | | |
| clono- | 1219 | GYT | 1407 | NPNS | 1595 | CTKE | 1783 | TGSS | 1971 | VNN | 2159 | COSY |
| type538 | | FTG | | GG | | GLVV | | SNIG | | NRP | | DSSL |
| | | Y | | | | RPDW | | AGYD | | S | | SGPV |
| | | | | | | FDPW | | VH | | | | F |
| clono- | 1220 | GDS | 1408 | YYRS | 1596 | CARK | 1784 | TGTS | 1972 | DVS | 2160 | CCSY |
| type539 | | VSS | | KWY | | GRDV | | SDVG | | KRP | | AGSS |
| | | NSA | | | | FDIW | | GYNY | | S | | TYWV |
| | | | | | | | | VS | | | | F |
| clono- | 1221 | GYT | 1409 | SAYN | 1597 | CARE | 1785 | TGSS | 1973 | GNS | 2161 | CQSY |
| type540 | | FTS | | GN | | GSGS | | SNIG | | NRP | | DSSL |
| | | Y | | | | YSDA | | AGYD | | S | | SGSY |
| | | | | | | FDIW | | VH | | | | VF |
| clono- | 1222 | GFS | 1410 | YWND | 1598 | CTHT | 1786 | TLRS | 1974 | YKS | 2162 | CMIW |
| type541 | | ITT | | D | | EYRN | | GIHV | | DSD | | HSSA |
| | | SGV | | | | TWCV | | DTSR | | KHQ | | IVF |
| | | | | | | DYW | | IY | | DS | | |
| clono- | 1223 | GFS | 1411 | YWND | 1599 | CAHS | 1787 | TLRS | 1975 | YKS | 2163 | CMIW |
| type542 | | LST | | D | | PYTS | | GINV | | DSD | | HSSA |
| | | SGV | | | | GWPF | | GSYR | | KQQ | | SVF |
| | | | | | | DYW | | IY | | GS | | |
| clono- | 1224 | GYT | 1412 | NPNS | 1600 | CARV | 1788 | TLRS | 1976 | YKS | 2164 | CMIW |
| type543 | | FTS | | GN | | SYSS | | GINV | | DSD | | HSSA |
| | | Y | | | | SWSL | | GTYR | | KQQ | | WVF |
| | | | | | | FDYW | | IY | | GS | | |
| clono- | 1225 | GFS | 1413 | DWDD | 1601 | CARI | 1789 | QGDS | 1977 | GKN | 2165 | CNSR |
| type548 | | LST | | D | | RGVG | | LRSY | | NRE | | DSSG |
| | | SGM | | | | ALDG | | YAS | | S | | NHLV |
| | | | | | | FDFW | | | | | | F |
| clono- | 1226 | GYT | 1414 | SGYK | 1602 | CARP | 1790 | QGDS | 1978 | GKN | 2166 | CNSR |
| type549 | | FTS | | GN | | LDYG | | LRSY | | NRP | | DSSG |
| | | Y | | | | DYEG | | YAS | | S | | NHLV |
| | | | | | | WFDP | | | | | | F |
| | | | | | | W | | | | | | |
| clono- | 1227 | GDT | 1415 | SAYN | 1603 | CARE | 1791 | QGDS | 1979 | GKN | 2167 | CNSR |
| type550 | | FTN | | GN | | GRTN | | LRSY | | NRP | | DSSG |
| | | C | | | | YFYY | | YAS | | S | | NHWV |
| | | | | | | YMDV | | | | | | F |
| | | | | | | W | | | | | | |
| clono- | 1228 | GFT | 1416 | SKNS | 1604 | CAKD | 1792 | QGDS | 1980 | GKN | 2168 | CNSR |
| type552 | | FDD | | GS | | ITAS | | LRSY | | NRP | | DSSG |
| | | Y | | | | GDYY | | YAS | | S | | NHLV |
| | | | | | | YMDV | | | | | | F |
| | | | | | | W | | | | | | |
| clono- | 1229 | GFT | 1417 | SSSS | 1605 | CARD | 1793 | QGDS | 1981 | HKN | 2169 | CNSR |
| type553 | | FSS | | ST | | RVYN | | LRSY | | NRP | | DSSG |
| | | Y | | | | WNDG | | YAS | | S | | NHVV |
| | | | | | | AFDI | | | | | | F |
| | | | | | | W | | | | | | |
| clono- | 1230 | GFT | 1418 | SGSG | 1606 | CAKD | 1794 | QGDS | 1982 | GKN | 2170 | CNSR |
| type554 | | FSS | | GS | | QRYN | | LRSY | | NRP | | DSSG |
| | | Y | | | | WNSW | | YAS | | S | | NHLV |
| | | | | | | YFDL | | | | | | F |
| | | | | | | W | | | | | | |
| clono- | 1231 | GFT | 1419 | WYDG | 1607 | CARD | 1795 | SGDA | 1983 | EDS | 2171 | CYST |
| type555 | | FSS | | SN | | HGGV | | LPKK | | KRP | | DSSG |
| | | Y | | | | TTYN | | YAY | | S | | NHRV |
| | | | | | | WFDP | | | | | | F |
| | | | | | | W | | | | | | |
| clono- | 1232 | GYT | 1420 | NPNS | 1608 | CARD | 1796 | TGTS | 1984 | EVS | 2172 | CSSY |
| type559 | | FTG | | GG | | RMVR | | SDVG | | KRP | | AGSN |
| | | Y | | | | GVLD | | GYNY | | S | | NVVF |
| | | | | | | AFDI | | VS | | | | |
| | | | | | | W | | | | | | |
| clono- | 1233 | GFT | 1421 | SSSS | 1609 | CVRG | 1797 | TGTS | 1985 | EVS | 2173 | CSSY |
| type560 | | FSS | | ST | | YSSG | | SDVG | | KRP | | AGSN |
| | | Y | | | | WYNW | | GYNY | | S | | NLVF |
| | | | | | | YFDL | | VS | | | | |
| | | | | | | W | | | | | | |
| clono- | 1234 | GFT | 1422 | SSSG | 1610 | CARK | 1798 | ASST | 1986 | STS | 2174 | CLLY |
| type561 | | FSD | | ST | | VPGI | | GAVT | | NKH | | YGGA |
| | | Y | | | | AAAG | | SGYY | | S | | QLVF |
| | | | | | | AFDY | | PN | | | | |
| | | | | | | W | | | | | | |
| clono- | 1235 | GYT | 1423 | NTNT | 1611 | CARG | 1799 | TGSN | 1987 | GNS | 2175 | COSY |
| type562 | | FNS | | GN | | GYGY | | SNIG | | NRP | | DNSL |
| | | Y | | | | NFWI | | AGYD | | S | | SGSV |
| | | | | | | RFDP | | IH | | | | F |
| | | | | | | W | | | | | | |
| clono- | 1236 | GGS | 1424 | YYSG | 1612 | CASY | 1800 | SGDV | 1988 | KDS | 2176 | CYSA |
| type568 | | ISR | | S | | WNFD | | LAKK | | ERP | | ADNN |
| | | SSY | | | | YW | | FAR | | S | | LVF |
| clono- | 1237 | GGS | 1425 | YHSG | 1613 | CARV | 1801 | SGDA | 1989 | EDS | 2177 | CYST |
| type569 | | ISS | | S | | LGYS | | LPKK | | KRP | | DSSG |
| | | SF | | | | YGYR | | YAY | | S | | NHRV |
| | | | | | | RWFD | | | | | | F |
| | | | | | | PW | | | | | | |
| clono- | 1238 | GFT | 1426 | KSKS | 1614 | CTTE | 1802 | GGNN | 1990 | YDS | 2178 | CQVW |
| type573 | | FSN | | DGET | | GSFN | | FGSK | | DRP | | DSTS |
| | | A | | | | FYYF | | SVH | | S | | DHYV |
| | | | | | | MDVW | | | | | | F |
| clono- | 1239 | GGS | 1427 | YYSG | 1615 | CARD | 1803 | TGTS | 1991 | AVS | 2179 | CCSY |
| type576 | | ISS | | S | | PFYY | | SDVG | | KRP | | AGTI |
| | | Y | | | | DFSD | | AYNY | | S | | SWVF |
| | | | | | | YYYM | | VS | | | | |
| | | | | | | DVW | | | | | | |
| clono- | 1240 | GFT | 1428 | SGSG | 1616 | CAKN | 1804 | TGTS | 1992 | DVS | 2180 | CCSY |
| type577 | | FSS | | GS | | EARD | | SDVG | | KRP | | AGSS |
| | | Y | | | | YYGS | | GYNY | | S | | TYVF |
| | | | | | | GSFD | | VS | | | | |
| | | | | | | YW | | | | | | |
| clono- | 1241 | GFT | 1429 | NWNG | 1617 | CASL | 1805 | TGTS | 1993 | EVN | 2181 | CSSY |
| type578 | | FGD | | GS | | VGAT | | SDVG | | KRP | | AGSN |
| | | F | | | | DYYF | | GYNY | | S | | NWVF |
| | | | | | | YYMD | | VS | | | | |
| | | | | | | VW | | | | | | |
| clono- | 1242 | GDS | 1430 | YYRS | 1618 | CARK | 1806 | TGTS | 1994 | DVS | 2182 | CCSY |
| type579 | | VSS | | KWY | | WELL | | SDVG | | KRP | | AGSS |
| | | NSA | | | | DAFD | | GYNY | | S | | TWVF |
| | | | | | | IW | | VS | | | | |
| clono- | 1243 | GFT | 1431 | SSSS | 1619 | CARE | 1807 | TGSS | 1995 | GNS | 2183 | CQSY |
| type581 | | FSS | | ST | | ERDD | | SNIG | | NRP | | DSSL |
| | | Y | | | | YSNY | | AGYD | | S | | SGWV |
| | | | | | | GYFQ | | VH | | | | F |
| | | | | | | HW | | | | | | |
| clono- | 1244 | GDS | 1432 | YYMS | 1620 | CARG | 1808 | TGTS | 1996 | DVS | 2184 | CSSY |
| type582 | | VSS | | KWY | | DWNY | | SDVG | | NRP | | TSSS |
| | | NSA | | | | GVLD | | SYNR | | S | | TYVV |
| | | | | | | SW | | VS | | | | F |
| clono- | 1245 | GYT | 1433 | SAYN | 1621 | CARS | 1809 | TGSD | 1997 | DNI | 2185 | COSY |
| type583 | | FTT | | GN | | GYNW | | SNIG | | IRP | | DISL |
| | | Y | | | | NYDY | | AGYD | | S | | SGSV |
| | | | | | | YFMD | | VH | | | | VF |
| | | | | | | VW | | | | | | |
| clono- | 1246 | GFT | 1434 | NWNG | 1622 | CARD | 1810 | SGDK | 1998 | QDS | 2186 | CQAW |
| type586 | | FDD | | GS | | GCSS | | LGDK | | KRP | | DSST |
| | | Y | | | | TSCY | | YAC | | S | | AVF |
| | | | | | | GNWF | | | | | | |
| | | | | | | DPW | | | | | | |
| clono- | 1247 | GDS | 1435 | YYRS | 1623 | CARV | 1811 | SGDG | 1999 | EDS | 2187 | CYST |
| type587 | | VSS | | KWY | | DFGI | | LSKK | | KRP | | DSSG |
| | | NSA | | | | VGAI | | YAY | | S | | KIF |
| | | | | | | DYW | | | | | | |
| clono- | 1248 | AFT | 1436 | SSST | 1624 | CARD | 1812 | QGDS | 2000 | GKN | 2188 | CNSR |
| type588 | | FSN | | SY | | RDDF | | LRSY | | NRP | | DSSG |
| | | Y | | | | WSGY | | YAS | | S | | NHWV |
| | | | | | | SPYF | | | | | | F |
| | | | | | | DYW | | | | | | |
| clono- | 1249 | GFT | 1437 | SSSS | 1625 | CARE | 1813 | QGDS | 2001 | GKN | 2189 | CNSR |
| type589 | | FSS | | SY | | KYDI | | LRSY | | NRP | | DSSG |
| | | Y | | | | LTGY | | YAS | | S | | NHWV |
| | | | | | | SPYF | | | | | | F |
| | | | | | | DYW | | | | | | |
| clono- | 1250 | GFT | 1438 | KSKT | 1626 | CTTD | 1814 | SGDA | 2002 | EDS | 2190 | CYST |
| type596 | | FSN | | DGGT | | QVSG | | LPKK | | KRP | | DSSG |
| | | A | | | | SYGD | | YAY | | S | | NHRV |
| | | | | | | AFDI | | | | | | F |
| | | | | | | W | | | | | | |
| clono- | 1251 | GFT | 1439 | SGSE | 1627 | CAKR | 1815 | QGDS | 2003 | GKN | 2191 | CNSR |
| type598 | | FSS | | SS | | AGSG | | LRSY | | NRP | | DSSG |
| | | H | | | | TYYR | | YAS | | S | | NHWV |
| | | | | | | GYYF | | | | | | F |
| | | | | | | DYW | | | | | | |
| clono- | 1252 | GFT | 1440 | WYDG | 1628 | CAGT | 1816 | QGDS | 2004 | GKN | 2192 | CNSR |
| type599 | | FSS | | SN | | YYYD | | LRSY | | NRP | | DSSG |
| | | Y | | | | SSGY | | YAS | | S | | NHLV |
| | | | | | | LNYM | | | | | | F |
| | | | | | | DVW | | | | | | |
| clono- | 1253 | GGS | 1441 | HYSG | 1629 | CASE | 1817 | QGDS | 2005 | GKN | 2193 | CNSR |
| type600 | | ISS | | S | | GPYF | | LRSY | | NRP | | DSSG |
| | | SSY | | | | DYW | | YAS | | S | | NHWV |
| | | | | | | | | | | | | F |
| clono- | 1254 | GYS | 1442 | SGYN | 1630 | CARA | 1818 | TGTS | 2006 | EVS | 2194 | CSSY |
| type601 | | FSS | | GN | | YCGG | | SDVG | | NRP | | TSSS |
| | | Y | | | | DCYY | | GYNY | | S | | TVVF |
| | | | | | | SNAF | | VS | | | | |
| | | | | | | DAW | | | | | | |
| clono- | 1255 | GFT | 1443 | KSKT | 1631 | CTTD | 1819 | TGTS | 2007 | DVS | 2195 | CCSY |
| type602 | | FSN | | DGGT | | RVTI | | SDVG | | KRP | | AGSS |
| | | A | | | | FGLA | | GYNY | | S | | TWVF |
| | | | | | | RMDV | | VS | | | | |
| | | | | | | W | | | | | | |
| clono- | 1256 | GFT | 1444 | SSGS | 1632 | CARD | 1820 | QGDS | 2008 | GRN | 2196 | CYSR |
| type607 | | FST | | ST | | PTTI | | LRSY | | NRP | | DSSG |
| | | Y | | | | FGVV | | YAT | | S | | NHLV |
| | | | | | | PYYY | | | | | | F |
| | | | | | | MDVW | | | | | | |
| clono- | 1257 | GFT | 1445 | KSKT | 1633 | CTTD | 1821 | GGNN | 2009 | YDS | 2197 | CQVW |
| type608 | | FSN | | DGGT | | RDYY | | IGSK | | DRP | | DSSS |
| | | A | | | | GSGS | | SVH | | S | | DHRV |
| | | | | | | YYFD | | | | | | F |
| | | | | | | YW | | | | | | |
| clono- | 1258 | GGS | 1446 | YYSG | 1634 | CARE | 1822 | QGDS | 2010 | GKN | 2198 | CNSR |
| type610 | | ITT | | N | | DLIG | | LRSY | | NRP | | DSSG |
| | | RSY | | | | NDYW | | YAS | | S | | NHLV |
| | | | | | | | | | | | | F |
| clono- | 1259 | GDS | 1447 | YYRS | 1635 | CSRD | 1823 | QGDS | 2011 | GKN | 2199 | CNSR |
| type611 | | VSS | | KWY | | RLIV | | LRSY | | KRP | | DSNG |
| | | NSA | | | | GASY | | YAS | | S | | NHWV |
| | | | | | | FDLW | | | | | | F |
| clono- | 1260 | GFT | 1448 | NWNG | 1636 | CARE | 1824 | TGTS | 2012 | DVS | 2200 | CCSY |
| type612 | | FDD | | GS | | KAPA | | SDVG | | KRP | | AGSS |
| | | Y | | | | HRSS | | GYNY | | S | | WVF |
| | | | | | | WSWY | | VS | | | | |
| | | | | | | FDLW | | | | | | |
| clono- | 1261 | GYT | 1449 | NPTS | 1637 | CTRE | 1825 | TGSS | 2013 | GNS | 2201 | CQSY |
| type613 | | FTG | | GV | | GIAA | | SNIG | | NRP | | DGTL |
| | | N | | | | ANPG | | ARYD | | S | | GGWI |
| | | | | | | YFYY | | VH | | | | F |
| | | | | | | MDVW | | | | | | |
| clono- | 1262 | GYT | 1450 | NPNS | 1638 | CARC | 1826 | QGDS | 2014 | GKN | 2202 | CNSR |
| type616 | | FTD | | GG | | DMVR | | LRSY | | NRP | | DSSG |
| | | Y | | | | GVID | | YAS | | S | | NHWV |
| | | | | | | HYYN | | | | | | F |
| | | | | | | YMDV | | | | | | |
| | | | | | | W | | | | | | |
| clono- | 1263 | GGS | 1451 | FYSG | 1639 | CVRQ | 1827 | TGTS | 2015 | DVS | 2203 | CCSY |
| type617 | | ISS | | S | | TYDS | | SDVG | | KRP | | AGSS |
| | | RSY | | | | WTGY | | GYNY | | S | | WVF |
| | | | | | | SFFY | | VS | | | | |
| | | | | | | FDYW | | | | | | |
| clono- | 1264 | GFT | 1452 | RSKA | 1640 | CTRP | 1828 | SGDK | 2016 | QDS | 2204 | CQAW |
| type622 | | FSG | | NSYA | | MITF | | LGDK | | KRP | | DSST |
| | | S | | | | GGVI | | YAC | | S | | VIF |
| | | | | | | VYDA | | | | | | |
| | | | | | | FDIW | | | | | | |
| clono- | 1265 | GGS | 1453 | NHSG | 1641 | CARG | 1829 | SGDA | 2017 | EDN | 2205 | CYST |
| type625 | | FSG | | S | | WGSS | | LPKK | | KRP | | DSSG |
| | | Y | | | | SWYY | | YAY | | S | | NHRV |
| | | | | | | FDYW | | | | | | F |
| clono- | 1266 | GGS | 1454 | NRSG | 1642 | CARG | 1830 | QGDS | 2018 | GKN | 2206 | CNSR |
| type626 | | FSG | | S | | IFGV | | LRNY | | NRP | | DSSG |
| | | Y | | | | GGNW | | YAS | | S | | NHLV |
| | | | | | | FDPW | | | | | | F |
| clono- | 1267 | GGS | 1455 | YYSG | 1643 | CARY | 1831 | TGTS | 2019 | EVS | 2207 | CSSY |
| type629 | | ISS | | S | | SSSW | | SDVG | | KRP | | AGSN |
| | | SSY | | | | SGFD | | GYNY | | S | | NF |
| | | | | | | YW | | VS | | | | |
| clono- | 1268 | GGS | 1456 | YYSG | 1644 | CARG | 1832 | QGDS | 2020 | GKN | 2208 | CNSR |
| type630 | | ISS | | S | | GSYY | | LRTY | | KRP | | DSSG |
| | | SSY | | | | VYFD | | YAS | | S | | NHWV |
| | | | | | | YW | | | | | | F |
| clono- | 1269 | GFT | 1457 | NQDG | 1645 | CSRD | 1833 | GGDN | 2021 | DDS | 2209 | CQVW |
| type634 | | FRS | | SE | | TDCS | | IGIK | | DRP | | DSSS |
| | | Y | | | | STSC | | NVH | | S | | DHVV |
| | | | | | | YFNW | | | | | | F |
| | | | | | | NPFF | | | | | | |
| | | | | | | DYW | | | | | | |
| clono- | 1270 | GGS | 1458 | FYSG | 1646 | CARG | 1834 | SGDK | 2022 | QDI | 2210 | CQAW |
| type638 | | INS | | F | | GYSY | | LGDK | | KRP | | DSST |
| | | SNF | | | | GLNW | | YTC | | S | | VVF |
| | | | | | | FDPW | | | | | | |
| clono- | 1271 | GGS | 1459 | YYSG | 1647 | CART | 1835 | QGDS | 2023 | GKN | 2211 | CNSR |
| type641 | | ISS | | S | | YYDF | | LRSY | | NRP | | DSSG |
| | | SSY | | | | WSGY | | YAS | | S | | NHVV |
| | | | | | | LNWF | | | | | | F |
| | | | | | | DPW | | | | | | |
| clono- | 1272 | GGS | 1460 | NRGG | 1648 | CARW | 1836 | TGTS | 2024 | EVS | 2212 | CSSY |
| type644 | | FSG | | S | | RNYY | | SDVG | | KRP | | AGSN |
| | | Y | | | | DSSG | | GYNY | | S | | NWVF |
| | | | | | | SPYW | | VS | | | | |
| | | | | | | YFDL | | | | | | |
| | | | | | | W | | | | | | |
| clono- | 1273 | GGS | 1461 | YYSG | 1649 | CARQ | 1837 | TGTS | 2025 | EVS | 2213 | CSSY |
| type646 | | ISS | | S | | GRIT | | SDVG | | NRP | | TSSS |
| | | SGY | | | | MVRG | | GYNY | | S | | TLVF |
| | | | | | | VIPF | | VS | | | | |
| | | | | | | DYW | | | | | | |
| clono- | 1274 | GGS | 1462 | NHSG | 1650 | CAGG | 1838 | GSST | 2026 | DTS | 2214 | CLLS |
| type647 | | FSG | | S | | YCSS | | GAVT | | NKH | | YSGA |
| | | Y | | | | TSCR | | SGHY | | S | | RVF |
| | | | | | | YNWN | | PY | | | | |
| | | | | | | YGGW | | | | | | |
| | | | | | | FDPW | | | | | | |

TABLE 6
Full Heavy Chain (HC) and Light Chain (LC) Sequences for Anti-CD131 Antibodies

| | SEQ | | SEQ | |
|---|---|---|---|---|
| clonotype | ID | | ID | |
| _id | NO | Full HC AA Sequence | NO | Full LC AA Sequence |
| clonotype | 523 | QLQLQESGPGLVKPSETLSLTCTV | 652 | QSALTQPASVSGSPGQSITISCT |
| 8 | | SGGSISSSSYYWGWIRQPPGKGLE | | GTSSDVGGYNYVSWYQQHPGKAP |
| | | WIGSIYYSGSTYYNPSLKSRVTIS | | KLMIYEVSNRPSGVSNRFSGSKS |
| | | VDTSKNQFSLKLSSVTAADTAVYY | | GNTASLTISGLQAEDEADYYCSS |
| | | CASLTGDRFDYWGQGTLVTVSS | | YTSSSTVVFGGGTKLTVL |
| clonotype | 524 | EVQLVESGGGLVKPGGSLRLSCAA | 653 | SYELTQPPSVSVSPGQTARITCS |
| 11 | | SGFTFSNAWMSWVRQAPGKGLEWV | | GDALPKKYAYWYQQKSGQAPVLV |
| | | GRIKSKTDGGTTDYAAPVKGRFTI | | IYEDSKRPSGIPERFSGSSSGTM |
| | | SRDDSKNTLYLQMNSLKTEDTAVY | | ATLTISGAQVEDEADYYCYSTDS |
| | | YCTGRPSIAARHFDYWGQGTLVTV | | SGNHSVFGTGTKVTVL |
| | | SS | | |
| clonotype | 525 | EVQLVESGGGVVRPGGSLRLSCAA | 654 | QSVLTQPPSVSGAPGQRVTISCT |
| 14 | | SGFTFDDYGMSWVRQAPGKGLEWV | | GSSSNIGAGYDVHWYQQLPGTAP |
| | | SGINWNGGSTGYADSVKGRFTISR | | KLLIYGNSNRPSGVPDRESGSKS |
| | | DNAKNSLYLQMNSLRAEDTALYYC | | GTSASLAITGLQAEDEADYYCQS |
| | | ARERLTIFGVVNYYMDVWGKGTTV | | YDSSLSGWVFGGGTKLTVL |
| | | TVSS | | |
| clonotype | 526 | EVQLVESGGGLVQPGGSLRLSCAA | 655 | SYELTQPSSVSVSPGQTARITCS |
| 15 | | SGFTFSSYSMNWVRQAPGKGLEWV | | GDVLAKKYARWFQQKPGQAPVLV |
| | | SYISSSSSTIYYADSVKGRFTISR | | IYKDSERPSGIPERFSGSSSGTT |
| | | DNAKNSLYLQMNSLRDEDTAVYYC | | VTLTISGAQVEDEADYYCYSAAD |
| | | ARDGWDYWGQGTLVTVSS | | NNRVFGGGTKLTVL |
| clonotype | 527 | EVQLVESGGGLVQPGGSLKLSCAA | 656 | SYELTQPPSVSVSPGQTARITCS |
| 16 | | SGFTFSSSDMHWVRQTTGKGLEWV | | GDALPKKYAYWYQQKSGQAPVLV |
| | | SAIYTTGDTYYPGSVKGRFTISRE | | IYEDSKRPSGIPERFSGSSSGTM |
| | | NAKNSLYLQMNSLRAGDTAVYYCA | | ATLTISGAQVEDEADYYCYSTDS |
| | | RGYSGSYYGDFDYWGQGTLVTVSS | | SGNRVFGGGTKLTVL |
| clonotype | 528 | EVQLVESGGGLVQPGGSLRLSCAA | 657 | QSVLTQPPSVSGAPGQRVTISCT |
| 17 | | SGFTFSSYAMSWVRQAPGKGLEWV | | GSSSNIGAGYDVHWYQQLPGTAP |
| | | SAISGSGGSTYYADSVKGRFTISR | | KLLIYGNSNRPSGVPDRFSGSKS |
| | | DNSKNTLYLQMNSLRAEDTAVYYC | | GTSASLAITGLQAEDEADYYCQS |
| | | AKKPPRDSAFDYWGQGTLVTVSS | | YDSSLSGSVFGGGTKLTVL |
| clonotype | 529 | QVQLVQSGAEVKKPGASVKVSCKA | 658 | QSALTQPPSASGSPGQSVTISCT |
| 25 | | SGYTFTSYGISWVRQAPGQGLEWM | | GTSSDVGGYNYVSWYQQHPGKAP |
| | | GWISAYNGNTNYAQKLQGRVTMTT | | KLMIYEVSKRPSGVPDRFSGSKS |
| | | DTSTSTAYMELRSLRSDDTAVYYC | | GNTASLTVSGLQAEDEADYYCSS |
| | | ARENSGSYYWFDPWGQGTLVTVSS | | YAGSNNVVFGGGTKLTVL |
| clonotype | 530 | EVQLVESGGGLVQPGGSLRLSCAA | 659 | QSALTQPASVSGSPGQSITISCT |
| 27 | | SGFTFSSYAMSWVRQAPGKGLEWV | | GTSSDVGGYNYVSWYQQHPGKAP |
| | | SAISGSGGSTYYADSVKGRFTISR | | KLMIYDVSKRPSGVSNRFSGSKS |
| | | DNSKNTLYLQMNSLRAEDTAVYYC | | GNTASLTISGLQAEDEADYYCCS |
| | | AKLEYSSPDYWGQGTLVTVSS | | YAGSSTLVFGGGTKLTVL |
| clonotype | 531 | QVQLQQWGAGLLKPSETLSLTCAV | 660 | SSELTQDPAVSVALGQTVRITCQ |
| 36 | | YGGSFSGYYWSWIRQPPGKGLEWI | | GDSLRSYYASWYQQKPGQAPVLV |
| | | GEINHSGSTNYNPSLKSRVTISVD | | IYGKNNRPSGIPDRFSGSSSGNT |
| | | TSKNQFSLKLSSVTAADTAVYYCA | | ASLTITGAQAEDEADYYCNSRDS |
| | | REGLLVGATLDAFDIWGQGTMVTV | | SGNHLVFGTGTKVTVL |
| | | SS | | |
| clonotype | 532 | QVQLVESGGGVVQPGRSLRLSCAA | 661 | SYVLTQPPSVSVAPGKTARITCG |
| 37 | | SGFTFSSYGMHWVRQAPGKGLEWV | | GNNIGSKSVHWYQQKPGQAPVLV |
| | | AVIWYDGSNKYYADSVKGRFTISR | | IYYDSDRPSGIPERFSGSNSGNT |
| | | DNSKNTLYLQMNSLRAEDTAVYYC | | ATLTISRVEAGDEADYYCQVWDS |
| | | ARDTGITMVRGVFDYWGQGTLVTV | | SSDHPVFGGGTKLTVL |
| | | SS | | |
| clonotype | 533 | QVQLVQSGAEVKKPGASVKVSCKA | 662 | QSVLTQPPSASGTPGQRVTISCS |
| 44 | | SGYTFTSYGISWVRQAPGQGLEWM | | GSSSNIGSNTVNWYQQLPGTAPK |
| | | GWISAYNGNTNYAQKFQGRVTMTT | | LLIYSNNQRPSGVPDRFSGSKSG |
| | | DTSTNTAYMELRSLRSDDKAVFYC | | TSASLAISGLQSEDEADYYCAAW |
| | | ARDRTGISAAGPSNWFDPWGQGTL | | DDSLNGPVFGGGTKLTVL |
| | | VTVSS | | |
| clonotype | 534 | QVQLQQWGAGLLKPSETLSLTCAV | 663 | QSALTQPPSASGSPGQSVTISCT |
| 45 | | YGGSFSGYYWSWIRQPPGKGLEWI | | GTSSDVGGYNYVSWYQQHPGKAP |
| | | GEINHSGSTNYNPSLKSRVTISVD | | KLMIYEVSKRPSGVPDRFSGSKS |
| | | TSKNQFSLKLSSVTAADTAVYYCA | | GNTASLTVSGLQAEDEADYYCSS |
| | | RTSLAAADFDYWGQGTLVTVSS | | YAGSNNYVFGTGTKVTVL |
| clonotype | 535 | EVQLVESGGGLIQPGGSLRLSCAA | 664 | QSVLTQPPSVSEAPRQRVTISCS |
| 47 | | SGFTVSSNYMSWVRQAPGKGLEWV | | GSSSNIGNNAVNWYQQLPGKAPK |
| | | SVIYSGGSTYYADSVKGRFTISRD | | LLIYYDDLLPSGVSDRFSGSKSG |
| | | NSKNTLYLQMNSLRAEDTAVYYCA | | TSASLAISGLQSEDEADYYCAAW |
| | | RAPDYYGSGSLFDYWGQGTLVTVS | | DDRLNGPVFGGGTKLTVL |
| | | S | | |
| clonotype | 536 | EVQLVESGGGLVKPGGSLRLSCIA | 665 | SYELTQPLSVSVALGQTARITCG |
| 52 | | SGFTFSTYSMNWVRQAPGKGLEWV | | GNNIGSKNVHWYQQKPGQAPVLV |
| | | SSISGSSSYIYYSDSVKGRFTISR | | IYRDSNRPSGIPERFSGSNSGNT |
| | | DNAKNSLYLQLNSLRAEDTAVYYC | | ATLTISRAQAGDEADYYCQVWDS |
| | | ASHWGHFDYWGRGTLVTVSS | | STVVFGGGTKLTVL |
| clonotype | 537 | EVQLVESGGGLVQPGGSLRLSCAA | 666 | SYELTQPPSVSVSPGQTARITCS |
| 115 | | SGFTFSSYDMHWVRQATGKGLEWV | | GDALPKKYAYWYQQKSGQAPVLV |
| | | SAIGTAGDTYYPGSVKGRFTISRE | | IYEDSKRPSGIPERFSGSSSGTM |
| | | NAKNSLYLQMNSLRAGDTAVYYCA | | ATLTISGAQVEDEADYYCYSTDS |
| | | RDRTLDYWGQGTLVTVSS | | SGNHWVFGGGTKLTVL |
| clonotype | 538 | QVQLVQSGAEAKKPGASVKVSCMA | 667 | QSALTQPPSASGSPGQSVTISCT |
| 116 | | SGYTFTTYGISWVRQAPGQGLEWM | | GTSSDVGGYNYVSWYQQHPGKAP |
| | | GWISAYNGNTKYAQKLQGRVTMTT | | KLMIYEVIKRPSGVPDRFSGSKS |
| | | DTSTRTAYMELRSLRSDDTAVYYC | | GNTASLTVSGLQAEDEADYYCSA |
| | | ARQIGDYWGQGTLVTVSS | | YAGSNNVVFGGGTQLTVL |
| clonotype | 539 | EVQLVESGGGLVQTGGSLKLSCAA | 668 | SYELTQPPSVSVSPGQTASITCS |
| 118 | | SGFTFSVSPIHWVRQASGKGLEWV | | GDKLGDKYACWYQQKPGQSPVLV |
| | | GRIRSKANSYATAYGASVKGRFTI | | IYQDSKRPSGIPERFSGSNSGNT |
| | | SRDDSKNTAYLQMNSLKTEDTAVY | | ATLTISGTQAMDEADYYCQAWDS |
| | | YCTGPFDNWGQGTLVTVSS | | STGVFGGGTKLTVL |
| clonotype | 540 | EVQLVESGGGLVKPGGSLRLSCAA | 669 | SYELTQPPSVSVSPGQTARITCS |
| 119 | | SGFTFSNAWMSWVRQAPGKGLEWV | | ADALPNQYAYWYQQKPGQAPVLV |
| | | GRIKSKTDGGTTDYTAPVKGRFTI | | IYKDSERPSGIPERFSGSSSGTT |
| | | SRDDSKNTLYLQMNSLRTEDTAVY | | VTLTISGVQAEDEADYYCQSADS |
| | | YCTTGGHYWGQGTLVTVSS | | SGTWVFGGGTKLTVL |
| clonotype | 541 | EVQLVESGGGLVQPGRSLRLSCAA | 670 | SYELTQPPSVSVSPGQTARITCS |
| 122 | | SGFTFDDYAMHWVRQAPGKGLEWV | | GDALPKKYAYWYQQKSGQAPVLV |
| | | SGISWNSGSIGYADSVKGRFTISR | | IYEDSKRPSGIPERFSGSSSGTM |
| | | DNAKNSLYLQMNSLRAEDTALYYC | | ATLTISGAQVEDEADYYCYSTDS |
| | | AKARSFDIWGQGTMVTVSS | | SGNHRVFGGGTKLTVL |
| clonotype | 542 | EVQLVESGGGLVKPGGSLRLSCAA | 671 | SYELTQPPSVSVSPGQTARITCS |
| 123 | | SGFTFSDAWMYWDRQAPGKGLEWV | | GDALPKKYAYWYQQKSGQAPVLV |
| | | GRIKSKTDGGTTDYAAPVKGRFTI | | IYEDSKRPSGIPERFSGSSSGTM |
| | | SRDDSKNTLYLQMNSLKTEDTAVY | | ATLTISGAQVEDEADYYCYSIDS |
| | | YCRADMDVWGKGTTVTVSS | | SGNHRVFGGGTKLTVL |
| clonotype | 543 | EVQLVESGGGVVRPGGSLRLSCAA | 672 | SYELTQPPSVSVSPGQTARITCS |
| 124 | | SGFTFDDYGMSWVRQAPGKGLEWV | | GDALPKKYAYWYQQKSGQAPVLV |
| | | SGINWNGGSTGYADSVKGRFTISR | | IYEDSKRPSGIPERFSGSSSGTM |
| | | DNAKNSLYLQMNSLRAEDTALYYC | | ATLTISGAQVEDEADYYCYSTDS |
| | | ARDRGFDYWGQGTLVTVSS | | SGNHRVFGGGTKLTVL |
| clonotype | 544 | EVQLVESGGGLIQPGGSLRLSCAA | 673 | SYELTQPPSVSVSPGQTARITCS |
| 125 | | SGFTVSSNYMSWVRQAPGKGLEWV | | GDALPKKYAYWYQQKSGQAPVLV |
| | | SVIYSGGSTYYADSVKGRFTISRD | | IYEDSKRPSGIPERFSGSSSGTM |
| | | NSKNTLYLQMNSLRAEDTAVYYCA | | ATLTISGAQVEDEADYYCYSTDS |
| | | RGGDYFDYWGQGTLVTVSS | | SGNHRVFGGGTKLTVL |
| clonotype | 545 | QVQLVQSGAEVKKPGASVKVSCKT | 674 | QSALTQPASVSGSPGQSITISCT |
| 126 | | SGYTFTSFGISWVRQAPGQGLEWM | | GTSSDVGGYNYVSWYQQHPGKAP |
| | | GWISAYNDNINYAQKLQDRVTMTT | | KLMIYEVSDRPSGVSNRFSGSKS |
| | | DTSTSTACMELRSLRSDDTAVYFC | | GNTASLTISGLQAEDEADYYCSS |
| | | ARGASFDFWGQGTLVTVSS | | YTRSSTCVFGGGTKLTVL |
| clonotype | 546 | EVQLVESGGGLVQPGGSLKLSCAA | 675 | QSALTQPASVSGSPGQSITISCT |
| 127 | | SGFTFSGSAMHWVRQASGKGLEWV | | GTSSDVGGYNYVSWYQQHPGKAP |
| | | GRIRSKANSYATAYAASVKGRFTI | | KLMIYEVSNRPSGVSNRFSGSKS |
| | | SRDDSKNTAYLQMNSLKTEDTAVY | | GNTASLTISGLQAEDEADYYCSS |
| | | YCTGPFDYWGQGTLVTVSS | | YTSSSTWVFGGGTKLTVL |
| clonotype | 547 | EVQLVESGGGLIQPGGSLRLSCAA | 676 | QSALTQPASVSGSPGQSITISCT |
| 128 | | SGFTVSSNYMSWVRQAPGKGLEWV | | GTSSDVGGYNYVSWYQQHPGKAP |
| | | SVIYSGGSTYYADSVKGRFTISRD | | KLMIYDVSKRPSGVSNRFSGSKS |
| | | NSKNTLYLQMNSLRAEDTAVYYCA | | GNTASLTISGLQAEDEADYYCCS |
| | | RSFDAFDIWGQGTMVTVSS | | YAGSSTFVVFGGGTKLTVL |
| clonotype | 548 | EVQLVESGGGLVKPGGSLRLSCAA | 677 | SYELTQPSSVSVSPGQTARITCS |
| 130 | | SGFTFSSYSMNWVRQAPGKGLEWV | | GDVLAKKYARWFQQKPGQAPVLV |
| | | SSISSSSSYIYYADSVKGRFTISR | | IYKDSERPSGIPERFSGSSSGTT |
| | | DNAKNSLYLQMNSLRAEDTAVYYC | | VTLTISGAQVEDEADYYCYSAAD |
| | | AGLTGELDYWGQGTLVTVSS | | NNLVFGGGTKLTVL |
| clonotype | 549 | EVQLVESGGGLVQPGGSLRLSCAA | 678 | SYELTQPPSVSVSPGQTARITCS |
| 132 | | SGFTFSSYDMHWVRQATGKGLEWV | | ADALPKQYAYWYQQKPGQAPVLV |
| | | SAIGTAGDTYYPGSVKGRFTISRE | | IYKDSERPSGIPERFSGSSSGTT |
| | | NAKNSLYLQMNSLRAGDTAVYYCA | | VTLTISGVQAEDEADYYCQSADS |
| | | RWGTGGFDYWGQGTLVTVSS | | SGTWVFGGGTKLTVL |
| clonotype | 550 | EVQLVESGGGLVKPGGSLRLSCAA | 679 | SYELTQPPSVSVSPGQTARITCS |
| 133 | | SGFTFSSYSMNWVRQAPGKGLEWV | | GDALPKKYAYWYQQKSGQAPVLV |
| | | SSISSSSSYIYYADSVKGRFTISR | | IYEDSKRPSGIPERFSGSSSGTM |
| | | DNAKNSLYLQMNSLRAEDTAVYYC | | ATLTISGAQVEDEADYYCYSTDS |
| | | ARREGFFDYWGQGTLVTVSS | | SGNHRVFGGGTKLTVL |
| clonotype | 551 | EVQLVESGGGLVQPGGSLRLSCAA | 680 | SYELTQPPSVSVSPGQTARITCS |
| 134 | | SGFTFSSYWMSWVRQAPGKGLEWV | | GDALPKKYAYWYQQKSGQAPVLV |
| | | ANIKQDGSEKYYVDSVKGRFTISR | | IYEDSKRPSGIPERFSGSSSGTM |
| | | DNAKNSLYLQMNSLRAEDTAVYYC | | ATLTISGAQVEDEADYYCYSTDS |
| | | ARDQLAPDYWGQGTLVTVSS | | SGNHRVFGGGTKLTVL |
| clonotype | 552 | EVQLVESGGGLVQPGGSLRLSCAA | 681 | SYELTQPPSVSVSPGQTARITCS |
| 135 | | SGFTFSSYAMSWVRQAPGKGLEWV | | GDALPKKYAYWYQQKSGQAPVLV |
| | | SAISGSGGSTYYADSVKGRFTISR | | IYEDSKRPSGIPERFSGSSSGTM |
| | | DNSKNTLYLQMNSLRAEDTAVYYC | | ATLTISGAQVEDEADYYCYSTDS |
| | | AKDSSGFDYWGQGTLVTVSS | | SGNHRVFGGGTKLTVL |
| clonotype | 553 | EVQLVESGGGLVQPGGSLRLSCAA | 682 | SYELTQPPSVSVSPGQTARITCS |
| 136 | | SGFTFSSYAMSWVRQAPGKGLEWV | | GDALPKKYAYWYQQKSGQAPVLV |
| | | SAISGSGGSTYYADSVKGRFTISR | | IYEDSKRPSGIPERFSGSSSGTM |
| | | DNSKNTLYLQMNSLRAEDTAVYYC | | ATLTISGAQVEDEADYYCYSTDS |
| | | AKDPQFFDYWGQGTLVTVSS | | SGNHRVFGGGTKLTVL |
| clonotype | 554 | EVQLVESGGGLVQPGGSLRLSCAA | 683 | SYELTQPPSVSVSPGQTARITCS |
| 137 | | SGFTFSSYAMSWVRQAPGKGLEWV | | GDALPKKYAYWYQQKSGQAPVLV |
| | | SAISGSGGSTYYADSVKGRFTISR | | IYEDSKRPSGIPERFSGSSSGTM |
| | | DNSKNTLYLQMNSLRAEDTAVYYC | | ATLTISGAQVEDEADYYCYSTDS |
| | | AKDGTAFDIWGQGTMVTVSS | | SGNHRVFGGGTKLTVL |
| clonotype | 555 | QVQLVESGGGVVQPGRSLRLSCAA | 684 | SYELTQPLSVSVALGQTARITCG |
| 138 | | SGFTFSSYGMHWVRQAPGKGLEWV | | GNNIGSKNVHWYQQKPGQAPVLV |
| | | AVIWYDGSNKYYADSVKGRFTISR | | IYRDSNRPSGIPERFSGSNSGNT |
| | | DNSKNTLYLQMNSLRAEDTAVYYC | | ATLTISRAQAGDEADYYCQVWDS |
| | | ARDRGWGLDYWGQGTLVTVSS | | STGVFGGGTKLTVL |
| clonotype | 556 | QVQLVESGGGVVQPGRSLRLSCAA | 685 | SSELTQDPAVSVALGQTVRITCQ |
| 140 | | SGFPFSNSGMHWVRQAPGKGLEWV | | GDSLRSYYASWYQQKPGQAPVLV |
| | | TIISYDGNSKYYADSVKGRFTISR | | IYGKNNRPSGIPDRFSGSSSGNT |
| | | DNSKNTLYLQMNSLRTEDTAVYYC | | ASLTITGAQAEDEADYYCNSRDS |
| | | ARGELGDFDYWGRGTLVTVSS | | SGNHLVFGGGTKLTVL |
| clonotype | 557 | QVQLVQSGAEVKKPGASVKVSCKA | 686 | QTVVTQEPSLTVSPGGTVTLTCA |
| 141 | | SGYTFTGYYMHWVRQAPGQGLEWM | | SSTGAVTSGYYPNWFQQKPGQAP |
| | | GWINPNSGGTNYAQKFQGRVTMTR | | RALIYSTSNKHSWTPARFSGSLL |
| | | DTSISTAYMELSRLRSDDTAVYYC | | GGKAALTLSGVQPEDEAEYYCLL |
| | | ARVLELYFDYWGQGTLVTVSS | | YYGGAVVFGGGTKLTVL |
| clonotype | 558 | EVQLVESGGGLVKPGGSLRLSCAA | 687 | QSVLTQPPSASGTPGQRVTISCS |
| 143 | | SGFTFSNAWMSWVRQAPGKGLEWV | | GSSSNIGSNTVNWYQQLPGTAPK |
| | | GRIKSKTDGGTTDYAAPVKGRFTI | | LLIYSNNQRPSGVPDRFSGSKSG |
| | | SRDDSKNTLYLQMNSLKTEDTAVY | | TSASLAISGLQSEDEADYYCAAW |
| | | YCTTRSDFQHWGQGTLVTVSS | | DDSLNGWVFGGGTKLTVL |
| clonotype | 559 | QVQLVQSGAEVKKPGASVKVSCKA | 688 | SYELTQPPSVSVSPGQTASITCS |
| 145 | | SGYTFTSYDINWVRQATGQGLEWM | | GDKLGDKYACWYQQKPGQSPVLV |
| | | GWMNPNSGNTGYAQKFQGRVTMTR | | IYQDSKRPSGIPERFSGSNSGNT |
| | | NTSISTAYMELSSLRSEDTAVYYC | | ATLTISGTQAMDEADYYCQAWDS |
| | | ARDQELRVFDYWGQGTLVTVSS | | STVVFGGGTKLTVL |
| clonotype | 560 | QVQLVESGGGVVQPGRSLRLSCAA | 689 | SYELTQPPSVSVSPGQTASITCS |
| 146 | | SGFTFSSYGMHWVRQAPGKGLEWV | | GDKLGDKYACWYQQKPGQSPVLV |
| | | AVISYDGSNKYYADSVKGRFTISR | | IYQDSKRPSGIPERFSGSNSGNT |
| | | DNSKNTLYLQMNSLRAEDTAVYYC | | ATLTISGTQAMDEADYYCQAWDS |
| | | AKASGYGPFDYWGQGTLVTVSS | | STVVFGGGTKLTVL |
| clonotype | 561 | EVQLVQSGAEVKKPGESLKISCKG | 690 | SYELTQPPSVSVSPGQTASITCS |
| 147 | | SGYSFTSYWIGWVRQMPGKGLEWM | | GDKLGDKYACWYQQKPGQSPVLV |
| | | GIIYPGDSDTRYSPSFQGQVTISA | | IYQDSKRPSGIPERFSGSNSGNT |
| | | DKSISTAYLQWSSLKASDTAMYYC | | ATLTISGTQAMDEADYYCQAWDS |
| | | ARHSSSSHFDYWGQGTLVTVSS | | STVVFGGGTKLTVL |
| clonotype | 562 | EVQLVESGGGLVKPGGSLRLSCAA | 691 | SYELTQPPSVSVSPGQTARITCS |
| 148 | | SGFTFSSYSMNWVRQAPGKGLEWV | | GDALPKKYAYWYQQKSGQAPVLV |
| | | SSISSSSSYIYYADSVKGRFTISR | | IYEDSKRPSGIPERFSGSSSGTM |
| | | DNAKNSLYLQMNSLRAEDTAVYYC | | ATLTISGAQVEDEADYYCYSTDS |
| | | ARDRGNSLFDYWGQGTLVTVSS | | SGNHRVFGGGTKLTVL |
| clonotype | 563 | QVQLVQSGAEVKKPGASVKVSCKA | 692 | SYELTQPPSVSVSPGQTARITCS |
| 150 | | SGYTFTGYYMHWVRQAPGQGLEWM | | GDALPKKYAYWYQQKSGQAPVLV |
| | | GWINPNSGGTNYAQKFQGRVTMTR | | IYEDSKRPSGIPERFSGSSSGTM |
| | | DTSISTAYMELSRLRSDDTAVYYC | | ATLTISGAQVEDEADYYCYSTDS |
| | | ARDKSLEWFDYWGQGTLVTVSS | | SGNHRVFGGGTKLTVL |
| clonotype | 564 | EVQLVESGGGLVQPGGSLRLSCAA | 693 | SYELTQPPSVSVSPGQTARITCS |
| 151 | | SGFTFSSYDMHWVRQATGKGLEWV | | GDALPKKYAYWYQQKSGQAPVLV |
| | | SAIGTAGDTYYPGSVKGRFTISRE | | IYEDSKRPSGIPERFSGSSSGTM |
| | | NAKNSLYLQMNSLRAGDTAVYYCA | | ATLTISGAQVEDEADYYCYSTDS |
| | | RGDWNYGGFDYWGQGTLVTVSS | | SGNHRVFGGGTKLTVL |
| clonotype | 565 | EVQLVESGGGLVKPGGSLRLSCAA | 694 | SYELTQPPSVSVSPGQTARITCS |
| 152 | | SGFTFSNAWMSWVRQAPGKGLEWV | | GDALPKKYAYWYQQKSGQAPVLV |
| | | GRIKSKTDGGTTDYAAPVKGRFTI | | IYEDSKRPSGIPERFSGSSSGTM |
| | | SRDDSKNTLYLQMNSLKTEDTAVY | | ATLTISGAQVEDEADYYCYSTDS |
| | | YCTTAPDAFDIWGQGTMVTVSS | | SGNHRVFGGGTKLTVL |
| clonotype | 566 | EVQLVESGGGLVQPGGSLRLSCAA | 695 | SYELTQPPSVSVSPGQTARITCS |
| 153 | | SGFTFSSYAMSWVRQAPGKGLEWV | | GDALPKKYAYWYQQKSGQAPVLV |
| | | SAISGSGGSTYYADSVKGRFTISR | | IYEDSKRPSGIPERFSGSSSGTM |
| | | DNSKNTLYLQMNSLRAEDTAVYYC | | ATLTISGAQVEDEADYYCYSTDS |
| | | ASGITGTTGDYWGQGTLVTVSS | | SGNHRVFGGGTKLTVL |
| clonotype | 567 | EVQLVESGGGLVQPGGSLRLSCAA | 696 | SYELTQPPSVSVSPGQTARITCS |
| 154 | | SGFTFSSYAMSWVRQAPGKGLEWV | | GDALPKKYAYWYQQKSGQAPVLV |
| | | SAISGSGGSTYYADSVKGRFTISR | | IYEDSKRPSGIPERFSGSSSGTM |
| | | DNSKNTLYLQMNSLRAEDTAVYYC | | ATLTISGAQVEDEADYYCYSTDS |
| | | AKEGAHDAFDIWGQGTMVTVSS | | SGNHRVFGGGTKLTVL |
| clonotype | 568 | EVQLVESGGGLVQPGGSLRLSCAA | 697 | SYELTQPPSVSVSPGQTARITCS |
| 156 | | SGFTFSSYAMSWVRQAPGKGLEWV | | GDALPKKYAYWYQQKSGQAPVLV |
| | | SAISGSGGSTYYADSVKGRFTISR | | IYEDSKRPSGIPERFSGSSSGTM |
| | | DNSKNTLYLQMNSLRAEDTAVYYC | | ATLTISGAQVEDEADYYCYSTDS |
| | | AKDKGELPFDYWGQGTLVTVSS | | SGNHRVFGGGTKLTVL |
| clonotype | 569 | QVQLVESGGGVVQPGRSLRLSCAA | 698 | SSELTQDPAVSVALGQTVRITCQ |
| 157 | | SGFTFSSYGMHWVRQAPGKGLEWV | | GDSLRSYYASWYQQKPGQAPVLV |
| | | AVIWYDGSNKYYADSVKGRFTISR | | IYGKNNRPSGIPDRFSGSSSGNT |
| | | DNSKNTLYLQMNSLRAEDTAVYYC | | ASLTITGAQAEDEADYYCNSRDS |
| | | AKLGVRDYMDVWGKGTTVTVSS | | SGNHWVFGGGTKLTVL |
| clonotype | 570 | EVQLVESGGGVVRPGGSLRLSCAA | 699 | SSELTQDPAVSVALGQTVRITCQ |
| 158 | | SGFTFDDYGMSWVRQAPGKGLEWV | | GDSLRSYYASWYQQKPGQAPVLV |
| | | SGINWNGGSTGYADSVKGRFTISR | | IYGKNNRPSGIPDRFSGSSSGNT |
| | | DNAKNSLYLQMNSLRAEDTALYYC | | ASLTITGAQAEDEADYYCNSRDS |
| | | AREGGGWVFDYWGQGTLVTVSS | | SGNHWVFGGGTKLTVL |
| clonotype | 57 | EVQLVQSGAEVKKPGESLKISCKG | 700 | SYELTQPPSVSVSPGQTARITCS |
| 159 | | SGYSFTSYWIGWVRQMPGKGLEWM | | GDALPKKYAYWYQQKSGQAPVLV |
| | | GIIYPGDSDTRYSPSFQGQVTISA | | IYEDSKRPSGIPERFSGSSSGTM |
| | | DKSISTAYLQWSSLKASDTAMYYC | | ATLTISGAQVEDEADYYCYSTDS |
| | | ARGGGGDPFDYWGQGTLVTVSS | | SGNHRVFGGGTKLTVL |
| clonotype | 572 | QVQLVQSGAEVKKPGASVQVSCKA | 701 | QSALTQPASVSGSLGQSITISCT |
| 160 | | SGYTFTGYYIHWVRQAPGQGLEWM | | GTSSDVGGYNYVSWYQHHPGKAP |
| | | GWINPNSGGTNYAQKFQGRVIMTR | | KIMIYDVSNRPSGVSNRFSASKS |
| | | DTSISIAYIELSRLRSDDTAVYYC | | GNTASLTISGLQTEDEADYYCSS |
| | | ARPYNWNSFDYWGQGTLVTVSS | | YTTSSTWVFGGGTNLTVL |
| clonotype | 573 | EVQLVESGGDLVQPGRSLRLSCAA | 702 | QSALTQPASVSGSPGQSITISCT |
| 161 | | SGFTFDDHAIHWVRQAPGKGLEWV | | GTSSDVGGYNYVSWYQQHPGKAP |
| | | SGVTWNSNIIGYADSVKGRFTISR | | KLMIYEVSNRPSVISYRFSGSKS |
| | | DIAKNSLYLQMNSLRPEDTALYYC | | GNTASLTISGLQAEDEADYYCNS |
| | | AKDNDWNGFDYWGQGTLVTVSS | | YTTNTTRVFGGGTKLTVL |
| clonotype | 574 | EVQLVESGGGLVQPGRSLRLSCAA | 703 | QSALTQPASVSGSPGQSITISCT |
| 162 | | SGFTEDDYAMHWVRQAPGKGLEWV | | GTSSDVGGYNYVSWYQQHPGKAP |
| | | SGISWNSGSIGYADSVKGRFTISR | | KLMIYEVSNRPSGVSNRFSGSKS |
| | | DNAKNSLYLQMNSLRAEDTALYYC | | GNTASLTISGLQAEDEADYYCSS |
| | | AKDNWNYAFDIWGQGTMVTVSS | | YTSSSTRVFGGGTKLTVL |
| clonotype | 575 | EVQLVESGGGLVQPGGSLRLSCTA | 704 | QAVLTQPASLSASPGASASLTCT |
| 164 | | SGFTFSSYWMHWVRQAPGKGLVWV | | LRSGIYVGTYRIYWYQQKPGSPP |
| | | SRVNSDGGNTIYADSVKGRFTISR | | QYLLRYKSDSDKQQGSGVPSRES |
| | | DNAKNTLYLQMNSLRAEDTAIYYC | | GSKDVSANAGILLISGLQSEDEA |
| | | ARDLDWTLFDYWGQGTLVTVSS | | DYYCMTWHSSAVVFGGGTKLTVL |
| clonotype | 576 | EVQLVESGGGLVQPGGSLRLSCAA | 705 | SYELTQPPSVSVSPGQTASITCS |
| 165 | | SGFTFSSYWMSWVRQAPGKGLEWV | | GDKLGDKYACWYQQKPGQSPVLV |
| | | ANIKQDGSEKYYVDSVKGRFTISR | | IYQDSKRPSGIPERFSGSNSGNT |
| | | DNAKNSLYLQMNSLRAEDTAVYYC | | ATLTISGTQAMDEADYYCQAWDS |
| | | AGDYSNYGWFDPWGQGTLVTVSS | | STVFGGGTKLTVL |
| clonotype | 577 | QVQLVQSGAEVKKPGASVKVSCKA | 706 | SYELTQPPSVSVSPGQTASITCS |
| 166 | | SGYTFTSYDINWVRQATGQGLEWM | | GDKLGDKYACWYQQKPGQSPVLV |
| | | GWMNPNSGNTGYAQKFQGRVTMTR | | IYQDSKRPSGIPERFSGSNSGNT |
| | | NTSISTAYMELSSLRSEDTAVYYC | | ATLTISGTQAMDEADYYCQAWDS |
| | | ARARDSGYYMDVWGKGTTVTVSS | | STVVFGGGTKLTVL |
| clonotype | 578 | QVQLVESGGGVVQPGRSLRLSCAA | 707 | SYELTQPSSVSVSPGQTARITCS |
| 167 | | SGFTFSSYGMHWVRQAPGKGLEWV | | GDVLAKKYARWFQQKPGQAPVLV |
| | | AVIWYDGSNKYYADSVKGRFTISR | | IYKDSERPSGIPERFSGSSSGTT |
| | | DNSKNTLYLQMNSLRAEDTAVYYC | | VTLTISGAQVEDEADYYCYSAAD |
| | | ARATAMVTGIDYWGQGTLVTVSS | | NNWVFGGGTKLTVL |
| clonotype | 579 | EVQLVESGGGLVQPGGSLKLSCAA | 708 | SYELTQPSSVSVSPGQTARITCS |
| 168 | | SGFTFSGSAMHWVRQASGKGLEWV | | GDVLAKKYARWFQQKPGQAPVLV |
| | | GRIRSKANSYATAYAASVKGRFTI | | IYKDSERPSGIPERFSGSSSGTT |
| | | SRDDSKNTAYLQMNSLKTEDTAVY | | VTLTISGAQVEDEADYYCYSAAD |
| | | YCTGSSGSYFDYWGQGTLVTVSS | | NNLVFGGGTKLTVL |
| clonotype | 580 | EVQLVESGGGLVKPGGSLRLSCAA | 709 | SYELTQPPSVSVSPGQTARITCS |
| 169 | | SGFTFSSYSMNWVRQAPGKGLEWV | | GDALPKKYAYWYQQKSGQAPVLV |
| | | SSISSSSSYIYYADSVKGRFTISR | | IYEDSKRPSGIPERFSGSSSGTM |
| | | DNAKNSLYLQMNSLRAEDTAVYYC | | ATLTISGAQVEDEADYYCYSTDS |
| | | ARSPYNWNYVDYWGQGTLVTVSS | | SGNHRVFGGGTKLTVL |
| clonotype | 581 | QVQLVQSGAEVKKPGASVKVSCKV | 710 | SYELTQPPSVSVSPGQTARITCS |
| 170 | | SGYTLTELSMHWVRQAPGKGLEWM | | GDALPKKYAYWYQQKSGQAPVLV |
| | | GGFDPEDGETIYAQKFQGRVTMTE | | IYEDSKRPSGIPERFSGSSSGTM |
| | | DTSTDTAYMDLSSLRSEDTAVYYC | | ATLTISGAQVEDEADYYCYSTDS |
| | | ATEGPSTFSFDYWGQGTLVTVSS | | SGNHRVFGGGTKLTVL |
| clonotype | 582 | QVQLVQSGAEVKKPGASVKVSCKV | 711 | SSELTQDPAVSVALGQTVRITCQ |
| 171 | | SGYTLTELSMHWVRQAPGKGLEWM | | GDSLRSYYASWYQQKPGQAPVLV |
| | | GGFDPEDGETIYAQKFQGRVTMTE | | IYGKNNRPSGIPDRFSGSSSGNT |
| | | DTSTDTAYMDLSSLRSEDTAVYYC | | ASLTITGAQAEDEADYYCNSRDS |
| | | ATANWNDEAFDIWGQGTMVTVSS | | SGNHLVFGGGTKLTVL |
| clonotype | 583 | EVQLVESGGGLVQPGGSLRLSCAA | 712 | SYELTQPPSVSVSPGQTARITCS |
| 172 | | SGFTFSSYSMNWVRQAPGKGLEWV | | GDALPKKYAYWYQQKSGQAPVLV |
| | | SYISSSSSTIYYADSVKGRFTISR | | IYEDSKRPSGIPERFSGSSSGTM |
| | | DNAKNSLYLQMNSLRDEDTAVYYC | | ATLTISGAQVEDEADYYCYSTDS |
| | | ARDELTGDAFDIWGQGTMVTVSS | | SGNHRVFGGGTKLTVL |
| clonotype | 584 | EVQLVESGGGLVKPGGSLRLSCAA | 713 | SYELTQPPSVSVSPGQTARITCS |
| 173 | | SGFTFSNAWMSWVRQAPGKGLEWV | | GDALPKKYAYWYQQKSGQAPVLV |
| | | GRIKSKTDGGTTDYAAPVKGRFTI | | IYEDSKRPSGIPERFSGSSSGTM |
| | | SRDDSKNTLYLQMNSLKTEDTAVY | | ATLTISGAQVEDEADYYCYSTDS |
| | | YCTTEALGIFDYWGQGTLVTVSS | | SGNHRVFGGGTKLTVL |
| clonotype | 585 | EVQLVESGGGLVKPGGSLRLSCAA | 714 | QTVVTQEPSLTVSPGGTVTLTCA |
| 174 | | SGFTFSSYSMNWVRQAPGKGLEWV | | SSTGAVTSGYYPNWFQQKPGQAP |
| | | SSISSSSSYIYYADSVKGRFTISR | | RALIYSTSNKHSWTPARFSGSLL |
| | | DNAKNSLYLQMNSLRAEDTAVYYC | | GGKAALTLSGVQPEDEAEYYCLL |
| | | ARDGSSGFLFDYWGQGTLVTVSS | | YYGGAWVFGGGTKLTVL |
| clonotype | 586 | EVQLVESGGGLVQPGGSLRLSCAA | 715 | QSALTQPPSASGSPGQSVTISCT |
| 175 | | SGFTFSSYAMSWVRQAPGKGLEWV | | GTSSDVGGYNYVSWYQQHPGKAP |
| | | SAISGSGGSTYYADSVKGRFTISR | | KLMIYEVSKRPSGVPDRESGSKS |
| | | DNSKNTLYLQMNSLRAEDTAVYYC | | GNTASLTVSGLQAEDEADYYCSS |
| | | AKHYYDSRSFDYWGQGTLVTVSS | | YAGSNNLVFGGGTKLTVL |
| clonotype | 587 | QVQLQESGPGLVKPSGTLSLTCAV | 716 | QSALTQPPSVSGAPGQRVTISCT |
| 176 | | SGGSISSSDWWTWVRQPPGRGLEW | | GSSSNIGAGYDVHWFQQLPGTAP |
| | | IGEINHSGTTNYNPSLKSRVTISV | | KLLIYDNNNRPSGVPNRFSGSKS |
| | | DKSKNQFSLKLSSVTAADTAVYYC | | GTSASLAITGLQADFEADYYCQS |
| | | ARDFQGTGPFDYWGQGTLVTVSS | | YDGSLNGWVFGGGTKLTVL |
| clonotype | 588 | QVQLQQWGAGLLKPSETLSLTCAV | 717 | SSELTQDPAVSVALGQTVRITCQ |
| 178 | | FGGSFSGYYWSWIRQPPGKGLEWI | | GDSLRNYYASWYQQKPGQAPVLV |
| | | GEINHSGSTNYNPSLKSRVTISVD | | IYGKNNRPSGIPDRFSGSSSGNI |
| | | TSKNQFSLKLTSVTAADTTVYYCA | | ASLTITGAQAEDEADYYCKSRDR |
| | | RGRLYSGSFSFDYWGQGTLVTVSS | | SGNHWVFGGGTKVTVL |
| clonotype | 589 | EVQLVESGGGLVQPGGSLRLSCAA | 718 | SSELTQDPAVSVALGQTVRITCQ |
| 179 | | SGFTFSSYWMSWVRQAPGKGLEWV | | GDSLRSYYASWYQQKPGQAPVLV |
| | | ANIKQDGSEKYYVDSVKGRFTISR | | IYGKNNRPSGIPDRFSGSSSGNT |
| | | DNAKNSLYLQMNSLRAEDTAVYYC | | ASLTITGAQAEDEADYYCNSRDS |
| | | ARDGGYNWNFFDYWGQGTLVTVSS | | SGNHVVFGGGTKLTVL |
| clonotype | 590 | QITLKESGPMLVKPTQTLTLTCTF | 719 | SSELTQDPAVSVALGQTVRITCQ |
| 180 | | SGFSLSTSGVGVGWIRQPPGKALE | | GDSLRSYYASWYQQKPGQAPVLV |
| | | WLALIYWNDDKRYSPSLKSRLTIT | | IYGKNNRPSGIPDRFSGSSSGNT |
| | | RDTSKNQVVLTMTNMDPVDTATYY | | ASLTITGAQAEDEADYYCNSRDS |
| | | CTHRDAAMVYFDYWGQGTLVTVSS | | SGNHWVFGGGTKLTVL |
| clonotype | 591 | QVQLVQSGAEVKKPGASVKVSCKA | 720 | SYELTQPPSVSVSPGQTARITCS |
| 181 | | SGYTFTSYGISWVRQAPGQGLEWM | | GDALPKKYAYWYQQKSGQAPVLV |
| | | GWISAYNGNTNYAQKLQGRVTMTT | | IYEDSKRPSGIPERFSGSSSGTM |
| | | DTSTSTAYMELRSLRSDDTAVYYC | | ATLTISGAQVEDEADYYCYSTDS |
| | | ARWYYGSGSYFDYWGQGTLVTVSS | | SGNHRVFGGGTKLTVL |
| clonotype | 592 | EVQLVESGGGLVQPGGSRRLSCAA | 721 | SSELTQDPAVSVALGQTVRITCQ |
| 182 | | SGFTFSRYDMHWVRQGTGKGLEWV | | GDNLRNYSVSWCQQRPGQAPTLV |
| | | SGINTAGDTYYSGSVKGRFTISRE | | IFGKNNRPSGIPDRFSGSNSGNT |
| | | NAKNSLHLQMNSLRAGDTAVYYCA | | ASLTITGAQAEDEADYYCNSRDI |
| | | RGWNYGSGSCFDNWGQGTLVTVSS | | SGKHWVFGGGTKLTVL |
| clonotype | 593 | EVQLVESGGGLVQPGGSLRLSCAA | 722 | SYELTQPPSVSVSPGQTARITCS |
| 183 | | SGFTFSSSDMHWVRQAPGEGLEWV | | GDALPKKYAYWYQQKSGQAPVLV |
| | | SAIYTTGDTYYPGSVQGRFTISRE | | IYEDSKRPSGIPERFSGSSSGTM |
| | | NAKNSLYLQMNSLRAGDTAVYYCA | | ATLTISGAQVEDEADYYCYSTDS |
| | | RGFSGTYYGDFDYWGQGTLVTVSS | | SGNHWVFGGGTKLTVL |
| clonotype | 594 | EVQLVESGGGLVQPGGSLRLSCAA | 723 | SYELTQPPSVSVSPGQTARITCS |
| 184 | | SGFTFSSYSMNWVRQAPGKGLEWV | | GDALPKKYAYWYQQKSGQAPVLV |
| | | SYISSSSSTIYYADSVKGRFTISR | | IYEDSKRPSGIPERFSGSSSGTM |
| | | DNAKNSLYLQMNSLRDEDTAVYYC | | ATLTISGAQVEDEADYYCYSTDS |
| | | AREGEWEPLHMDVWGKGTTVTVSS | | SGNHRVFGGGTKLTVL |
| clonotype | 595 | EVQLVESGGGLVQPGGSLRLSCAA | 724 | SYELTQPPSVSVSPGQTARITCS |
| 185 | | SGFTFSSYAMSWVRQAPGKGLEWV | | GDALPKKYAYWYQQKSGQAPVLV |
| | | SAISGSGGSTYYADSVKGRFTISR | | IYEDSKRPSGIPERFSGSSSGTM |
| | | DNSKNTLYLQMNSLRAEDTAVYYC | | ATLTISGAQVEDEADYYCYSTDS |
| | | AKSLSGSYVYMDVWGKGTTVTVSS | | SGNHRVFGGGTKLTVL |
| clonotype | 596 | QVQLVESGGGVVQPGRSLRLSCAA | 725 | SSELTQDPAVSVALGQTVRITCQ |
| 186 | | SGFTFSSYGMHWVRQAPGKGLEWV | | GDSLRSYYASWYQQKPGQAPVLV |
| | | AVISYDGSNKYYADSVKGRFTISR | | IYGKNNRPSGIPDRFSGSSSGNT |
| | | DNSKNTLYLQMNSLRAEDTAVYYC | | ASLTITGAQAEDEADYYCNSRDS |
| | | AKGFLEWLLGFDYWGQGTLVTVSS | | SGNHWVFGGGTKLTVL |
| clonotype | 597 | QVQLVESGGGLVKPGGSLRLSCAA | 726 | SYVLTQPPSVSVAPGKTARITCG |
| 187 | | SGFTFSDYYMSWIRQAPGKGLEWV | | GNNIGSKSVHWYQQKPGQAPVLV |
| | | SYISSSGSTIYYADSVKGRFTISR | | IYYDSDRPSGIPERFSGSNSGNT |
| | | DNSKNTLYLQMNSLRAEDTAVYYC | | ATLTISRVEAGDEADYYCQVWDS |
| | | ARDGGSSGYYSDYWGQGTLVTVSS | | SSDHVVFGGGTKLTVL |
| clonotype | 598 | EVQLVESGGGLVQPGGSLRLSCAA | 727 | SSELTQDPAVSVALGQTVRITCQ |
| 188 | | SGFTFSEYWMHWVRQAPGKGLVWV | | GDSLRSYYANWYQQKPGQAPILV |
| | | ARINSDGSRTDYADSVKGRFTISR | | IYGKNNRPSGIPDRFSGSSSGNT |
| | | NNAKNRLNLQIDSLRAEDTAVYYC | | ASLTITGAQAEDESDYYCNSRDS |
| | | TRDLVYSSGWYDYWGQGTLVTVSS | | SGNHWVFGGGTKLTVL |
| clonotype | 599 | EVQLVESGGGLVQPGGSLRLSCAA | 728 | SSELTQDPAVSVALGQTVRITCQ |
| 189 | | SGFTFSSYWMHWVRQAPGKGLVWV | | GDSLRTYYASWYQQKPGQAPILV |
| | | SRINSDGSGTSYADSVKGRFTISR | | IYGKNNRPSGIPDRFSGSSSGNT |
| | | DNAKNTLYLQMNSLRAEDTAVYYC | | ASLTITGAQAEDEADYSCNSRDS |
| | | AREGIKASDAFDIWGQGTMVTVSS | | SGSHVVFGGGTKLTVL |
| clonotype | 600 | EVQLVESGGGLVQPGRSLRLSCAA | 729 | SYELTQPPSVSVSPGQTARITCS |
| 190 | | SGFTFDDYAMHWVRQAPGKGLEWV | | GDALPKKYAYWYQQKSGQAPVLV |
| | | SGISWNSGSIGYADSVKGRFTISR | | IYEDSKRPSGIPERFSGSSSGTM |
| | | DNAKNSLYLQMNSLRAEDTALYYC | | ATLTISGAQVEDEADYYCYSTDS |
| | | AKDIDPSITGTDYWGQGTLVTVSS | | SGNHSVVFGGGTKLTVL |
| clonotype | 601 | QVQLVESGGGLVKPGGSLRLSCAA | 730 | QTVVTQEPSLTVSPGGTVTLTCA |
| 191 | | SGFTFSDYYMSWIRQAPGKGLEWV | | SSTGAVTSGYYPNWFQQKPGQAP |
| | | SYISHSGTTVYYADSVKGRFTISR | | RALIYSTSNKHSWTPARFSGSLL |
| | | DNAKISLYLQMNSLRAEDTAVYYC | | GGKAALTLSGVQPEDEAEYYCLL |
| | | AGLRHFDWLGFDSWGQGTLVTVSS | | YYGGAWVFGGGTKLTVL |
| clonotype | 602 | EVQLVESGGGLVQPGGSLRLSCAA | 731 | QSALTQPASVSGSPGQSITISCT |
| 192 | | SGFTFSSYAMNWVRQAPGKGLEWV | | GTSSDVGGYNYVSWYQQHPGKAP |
| | | SIINDSGYSTYYADSVKGRFTISR | | KVIIYEVIIRPSGVSPRFSGSKS |
| | | DNSKNTLYLQMNSLRAEDTALYYC | | GKMASLTISGLQAEDEADYYCSS |
| | | AKEDNWNYGWFDPWGQGTLVTVSS | | YTSSSTWVFGGGTKLTVL |
| clonotype | 603 | EVQLVESGGGLVQPGGSLRLSCAA | 732 | QSALTQPASVSGSPGQSITISCT |
| 193 | | SGFTFSSYDMHWVRQATGKGLEWV | | GTSSDVGGYNYVSWYQQHPGKAP |
| | | SAIGTAGDTYYPGSVKGRFTISRE | | KLMIYEVSNRPSGVSNRFSGSKS |
| | | NAKNSLYLQMNSLRAGDTAVYYCA | | GNTASLTISGLQAEDEADYYCSS |
| | | REETGTTSWYFDLWGRGTLVTVSS | | YTSSSTLYVFGTGTKVTVL |
| clonotype | 604 | EVQLVESGGGLVQPGGSLRLSCAA | 733 | QSALTQPASVSGSPGQSITISCT |
| 194 | | SGFTFSSYSMNWVRQAPGKGLEWV | | GTSSDVGGYNYVSWYQQHPGKAP |
| | | SYISSSSSTIYYADSVKGRFTISR | | KLMIYEVSNRPSGVSNRFSGSKS |
| | | DNAKNSLYLQMNSLRDEDTAVYYC | | GNTASLTISGLQAEDEADYYCSS |
| | | ARGYSYGYWYFDLWGRGTLVTVSS | | YTSSSTPYVFGTGTKVTVL |
| clonotype | 605 | QVQLVQSGAEVKKPGASVKVSCKV | 734 | SYELTQPPSVSVSPGQTASITCS |
| 195 | | SGYTLTELSMHWVRQAPGKGLEWM | | GDKLGDKYACWYQQKPGQSPVLV |
| | | GGFDPEDGETIYAQKFQGRVTMTE | | IYQDSKRPSGIPERFSGSNSGNT |
| | | DTSTDTAYMDLSSLRSEDTAVYYC | | ATLTISGTQAMDEADYYCQAWDS |
| | | ATPYCSGGSCHFDYWGQGTLVTVS | | STVVFGGGTKLTVL |
| | | S | | |
| clonotype | 606 | EVQLVESGGGLVKPGGSLRLSCAA | 735 | SYELTQPPSVSVSPGQTARITCS |
| 196 | | SGFTFSSYSMNWVRQAPGKGLEWV | | GDALPKKYAYWYQQKSGQAPVLV |
| | | SSISSSSSYIYYADSVKGRFTISR | | IYEDSKRPSGIPERFSGSSSGTM |
| | | DNAKNSLYLQMNSLRAEDTAVYYC | | ATLTISGAQVEDEADYYCYSTDS |
| | | ARDDYGGNSVYFDYWGQGTLVTVS | | SGNHRVFGGGTKLTVL |
| | | S | | |
| clonotype | 607 | QVQLVESGGGAVQPGRSLRLSCVA | 736 | SYVLTQSPSMSVAPGKTARITCG |
| 198 | | SGFTFSNYDMHWVRQAPGKGLEWV | | GNNIGSKSVHWYQQRPGQAPVLV |
| | | AVIWSDGSNKYYSDSVKGRFTISR | | IYYDSDRPSGIPERFSGSNSGNT |
| | | DNSKNTLYLQMTSLSAEDSALSYC | | ATLTISRVEAGDEAVYYCQVWDS |
| | | VRAARYSGTYIFDYWGQGTLVTVS | | SSYHYVFGTGTKVAVL |
| | | S | | |
| clonotype | 608 | EVQLVESGGGLVKPGGSLRLSCAA | 737 | SYELTQPPSVSVSPGQTARITCS |
| 199 | | SGFTFSNAWMSWVRQAPGKGLEWV | | GDALPKKYAYWYQQKSGQAPVLV |
| | | GRIKSKTDGGTTDYAAPVKGRFAI | | IYEDSKRPSGIPERFSGSSSGTM |
| | | SRDDSKNTLYLQMNSLKTEDTAVY | | ATLTISGAQVEDEADYYCYSTDS |
| | | YCTTDPGYSYGVDYWGQGTLVTVS | | SGNHRVFGGGTKLTVL |
| | | S | | |
| clonotype | 609 | EVQLVQSGAEVKKPGESLKISCKG | 738 | SYELTQPPSVSVSPGQTARITCS |
| 200 | | SGYSFTSYWIGWVRQMPGKGLEWM | | GDALPKKYAYWYQQKSGQAPVLV |
| | | GIIYPGDSDTRYSPSFQGQVTISA | | IYEDSKRPSGIPERFSGSSSGTM |
| | | DKSISTAYLQWSSLKASDTAMYYC | | ATLTISGAQVEDEADYYCYSTDS |
| | | ARPEYSSSSGYFQHWGQGTLVTVS | | SGNHRVFGGGTKLTVL |
| | | S | | |
| clonotype | 610 | EVQLVESGGGLVQPGGSLRLSCAA | 739 | QTVVTQEPSLTVSPGGTVTLTCA |
| 201 | | SGFTFSSYWMSWVRQAPGKGLEWV | | SSTGAVTSGYYPNWFQQKPGQAP |
| | | ANIKQDGSEKYYVDSVKGRFTISR | | RALIYSTSNKHSWTPARFSGSLL |
| | | DNAKNSLYLQMNSLRAEDTAVYYC | | |
| | | AREYNWNYEDAFDIWGQGTMVTVS | | GGKAALTLSGVQPEDEAEYYCLL |
| | | S | | YYGGAQVFGGGTKLTVL |
| clonotype | 611 | QITLKESGPTLVKPTQTLTLTCTF | 740 | QSALTQPASVSGSPGQSITISCT |
| 202 | | SGFSLSTSGVGVGWIRQPPGKALE | | GTSSDVGGYNYVSWYQQHPGKAP |
| | | WLALIYWNDDKRYSPSLKSRLTIT | | KLMIYDVSKRPSGVSNRFSGSKS |
| | | KDTSKNQVVLTMTNMDPVDTATYY | | GNTASLTISGLQAEDEADYYCCS |
| | | CAHRRGSYSNWFDPWGQGTLVTVS | | YAGSSTWVFGGGTKLTVL |
| | | S | | |
| clonotype | 612 | QIQLVQSGAEVKKPGASVKVSCTA | 741 | QSVLSQPPSVSEAPRQRVTISCS |
| 203 | | SGYTFSSYGITWVRQAPGQGLEWM | | GSSSNIGNNAVNWYQKLPGKAPK |
| | | GWISAYNGNTHYAQNLQGRVTMTT | | LLISHDVLLSSGVSDRFSGSKSG |
| | | DTSTTTAYMDLRSLRSDDTAIYYC | | TSASLAISGLQSEDEADYYCAAW |
| | | ARTLFGVVKNWFDPWGQGTLVTVS | | DGRLNEWVFGGGTKLTVL |
| | | S | | |
| clonotype | 613 | QVQLVQSGASVKVSCKASGYTFTG | 742 | QSVLTQPPSASGTPGQRVTISCS |
| 204 | | YYMHWVRQAPGQGLEWMGWINPNS | | GSSSNIGSNTVNWYQQLPGTAPK |
| | | GGTNYAQKFQGRVTMTRDTSISTA | | LLIYSNNQRPSGVPDRESGSKSG |
| | | YMELSRLRSDDTAVYYCAREVLGG | | TSASLAISGLQSEDEADYYCAAW |
| | | GDCPFDYWGQGTLVTVSS | | DDSLNGVVFGGGTKLTVL |
| clonotype | 614 | QVQLVQSGAEVRKPVASVKVSCKA | 743 | QSVLTQPPSVSEAPRQRVTISCS |
| 205 | | SGYTFTDHSIHWVRQAPGQGLEWM | | GSISNIGNNAVSWYQQVPGKAPK |
| | | GSINPNSGGTNYAQKFQGRVTMTW | | LLIYYDDLLPSGVSDRFSGSRSV |
| | | DTYNSTAFMELSRLRSDDTAVYYC | | TSASLAISGLQSEDDADYYCTAW |
| | | ARSDGGSHYVFFDDWGQGTLVTVS | | DDRLNGPVFGGGTKLTVL |
| | | S | | |
| clonotype | 615 | EVQLVESGGGLVQPGRSLRLSCAA | 744 | QSALTQPPSASGSPGQSVTISCT |
| 206 | | SGFTFDDYAMHWVRQAPGKGLEWV | | GTSSDVGGYNYVSWYQQHPGKAP |
| | | SGISWNSGSIGYADSVKGRFTISR | | KLMIYEVSKRPSGVPDRFSGSKS |
| | | DNAKNSLYLQMNSLRAEDTALYYC | | GNTASLTVSGLQAEDEADYYCSS |
| | | AKDIAYSSSGHFDYWGQGTLVTVS | | YAGSNNLVFGGGTKLTVL |
| | | S | | |
| clonotype | 616 | QVQLQESGPGLVKPSGTLSLTCVV | 745 | QSVLTQPPSVSGAPGQRVTISCS |
| 207 | | SGGSITSSNWWSWVRQPPGKGLEW | | GSSSNIGAGYDVHWYQQLPGTGP |
| | | IGEIYHSGNTNYNPSLKSRVTISV | | KVLIYGNRNRPSGVPDRFSGSKS |
| | | DKSKNQFSLRLSSVTAADTAVYYC | | GTSASLVITGLQAEDEADYSCQS |
| | | ARAPLTGTTNWFDPWGQGTLVTVS | | YDSSLSGWVFGGGTKLTVL |
| | | S | | |
| clonotype | 617 | EVQLVESGGGLVKPGGSLRLSCAA | 746 | SYELTQPSSVSVSPGQTARITCS |
| 209 | | SGFTFSSYSMNWVRQAPGKGLEWV | | GDVLAKKYARWFQQKPGQAPVLV |
| | | SSISSSSSYIYYADSVKGRFTISR | | IYKDSERPSGIPERFSGSSSGTT |
| | | DNAKNSLYLQMNSLRAEDTAVYYC | | VTLTISGAQVEDEADYYCYSAAD |
| | | AGVLYYDSSGYPFDYWGQGTLVTV | | NNLVFGGGTKLTVL |
| | | SS | | |
| clonotype | 618 | QVQLVQSGSELKKPGASVKVSCKA | 747 | SYELTQPPSVSVSPGQTASITCS |
| 210 | | SGYTFTSYAMNWVRQAPGQGLEWM | | GDKLGDKYACWYQQKPGQSPVLV |
| | | GWINTNTGNPTYAQGFTGRFVFSL | | IYQDSKRPSGIPERFSGSNSGNT |
| | | DTSVSTAYLQISSLKAEDTAVYYC | | ATLTISGTQAMDEADYYCQAWDS |
| | | ARDPLAARPVGWFDPWGQGTLVTV | | STAVFGGGTKLTVL |
| | | SS | | |
| clonotype | 619 | EVQLVESGGGLVKPGGSLRLSCAA | 748 | SSELTQDPAVSVALGQTVRITCQ |
| 211 | | SGFTFSSYSMNWVRQAPGKGLEWV | | GDSLRSYYASWYQQKPGQAPVLV |
| | | SSISSSSSYIYYADSVKGRFTISR | | IYGKNNRPSGIPDRFSGSSSGNT |
| | | DNAKNSLYLQMNSLRAEDTAVYYC | | ASLTITGAQAEDEADYYCNSRDS |
| | | AREDGYSSGWNYFDYWGQGTLVTV | | SGNHWVFGGGTKLTVL |
| | | SS | | |
| clonotype | 620 | QVQLVQSGAEVKKPGASVKVSCKV | 749 | SYVLTQPPSVSVAPGKTARITCG |
| 212 | | SGYTLTELSMHWVRQAPGKGLEWM | | GNNIGSKSVHWYQQKPGQAPVLV |
| | | GGFDPEDGETIYAQKFQGRVTMTE | | IYYDSDRPSGIPERFSGSNSGNT |
| | | DTSTDTAYMDLSSLRSEDTAVYYC | | ATLTISRVEAGDEADYYCQVWDS |
| | | ATGGQTIVAARVFDYWGQGTLVTV | | SSDHVVFGGGTKLTVL |
| | | SS | | |
| clonotype | 621 | QVQLVQSGSELKKPGASVKVSCKA | 750 | SSELTQDPAVSVALGQTVRITCQ |
| 213 | | SGYTVTRHALNWVRQAPGQGLEWM | | GDSLRSYYASWYQQKPGQAPVLV |
| | | GWINTNTGTPTYAQGFIGRFVFTL | | IYGKNNRPSGIPDRFSGSSSGNT |
| | | DTSVSTAYLQINSLKAEDTAVYYC | | ASLTITGAQAEDEADYYCNSRDS |
| | | ARDQTPSDHYYYMDVWGKGTTVTV | | SGNHYVFGTGTKVTVL |
| | | SS | | |
| clonotype | 622 | LAHLVQSGAEVKRPGASVKVSCKA | 751 | SSELTQDPAVSVALGQTVRITCQ |
| 214 | | FGYAFRGQHIHWVRQAPGQGLEWM | | GDSLRSYYASWYQQKPGQAPVLV |
| | | GWIRPNSGDTNYSQKFQGRVTMTR | | IYGKNNRPSGIPDRFSGSSSGNT |
| | | DTSITTAYMELTRLRSDDSAVYYC | | ASLTITGAQAEDEADYYCNSRDS |
| | | ARDRGITMRLDNMDVWGKGTMVTV | | SGNHLVFGGGTKLTVL |
| | | SS | | |
| clonotype | 623 | EVQLVESGGTLVQPGGSLTLSCAA | 752 | QSALTQPASVSGSPGQSITISCT |
| 215 | | SGFTFSDSAMHWVRQASGKGLEWV | | GTSSDVGAYNYVSWYQQHPGKAP |
| | | GRIRGKPNTYATAYAASVKGRFTI | | KFMIYDVSKRPSGVSNRFSGSKS |
| | | SKDDSKNTAFLQMNSLKTEDRAVY | | GNTASLTISGLQAEDEADYYCCS |
| | | YCTRRYNWNDVGFDYWGQGTLVTV | | YAGSNTYVFGTGTRVTVL |
| | | SS | | |
| clonotype | 624 | QITLKESGPTLVKPTQTLTLTCTF | 753 | SYELTQPPSVSVSPGQTASITCS |
| 217 | | SGFSLSTSGVGVGWIRQPPGKALE | | GDKLGDKYACWYQQKPGQSPVLV |
| | | WLALIYWNDDKRYSPSLKSRLTIT | | IYQDSKRPSGIPERFSGSNSGNT |
| | | KDTSKNQVVLTMTNMDPVDTATYY | | ATLTISGTQAMDEADYYCQAWDS |
| | | CAHRPGITGNTGYFDYWGQGTLVT | | STVVFGGGTKLTVL |
| | | VSS | | |
| clonotype | 625 | QVQLVQSGAEVKKPGASVKVSCKA | 754 | SYELTQPPSVSVSPGQTASITCS |
| 218 | | SGYTFTSYGISWVRQAPGQGLEWM | | GDKLGDKYACWYQQKPGQSPVLV |
| | | GWISAYNGNTNYAQKLQGRVTMTT | | IYQDSKRPSGIPERFSGSNSGNT |
| | | DTSTSTAYMELRSLRSDDTAVYYC | | ATLTISGTQAMDEADYYCQAWDS |
| | | ARCRYSGSLTSYYMDVWGKGTTVT | | STVVFGGGTKLTVL |
| | | VSS | | |
| clonotype | 626 | EVQLVESGGGLVQPGRSLRLSCAA | 755 | SYELTQPPSVSVSPGQTASITCS |
| 219 | | SGFTEDDYAMHWVRQAPGKGLEWV | | GDKLGDKYACWYQQKPGQSPVLV |
| | | SGISWNSGSIGYADSVKGRFTISR | | IYQDSKRPSGIPERFSGSNSGNT |
| | | DNAKNSLYLQMNSLRAEDTALYYC | | ATLTISGTQAMDEADYYCQAWDS |
| | | AKDMITGTTNYYYMDVWGKGTTVT | | STVVFGGGTKLTVL |
| | | VSS | | |
| clonotype | 627 | EVQLVESGGGLVQPGRSLRLSCAA | 756 | SYELTQPLSVSVALGQTARITCG |
| 220 | | SGFTEDDYAMHWVRQAPGKGLEWV | | ENNIVNKNVHWYQQKPGQAPVLV |
| | | SGISRNSGSVGYADSVRGRFTISR | | IYRDGNRPSGIPERFSGSNSGNT |
| | | DNAKNSLYLQMNSLRAEDTALYYC | | ATLTISRAQAGDEADYYCQVWDN |
| | | AKGGYDFWSGYYPFDPWGQGTLVT | | NTPWVFGGGTKLTVL |
| | | VSS | | |
| clonotype | 628 | EVQLVESGGGLVKPGGSLRLSCAA | 757 | SYELTQPPSVSVSPGQTARITCS |
| 223 | | SGFTFSNAWMSWVRQAPGKGLEWV | | GDALPKKYAYWYQQKSGQAPVLV |
| | | GRIKSKTDGGTTDYAAPVKGRFTI | | IYEDSKRPSGIPERFSGSSSGTM |
| | | SRDDSKNTLYLQMNSLKTEDTAVY | | |
| | | YCTTEGTTVTTWAFDIWGQGTMVT | | ATLTISGAQVEDEADYYCYSTDS |
| | | VSS | | SGNHRVFGGGTKLTVL |
| clonotype | 629 | QVQLQQSGPGLVKPSQTLSLTCAI | 758 | SYELTQPPSVSVSPGQTARITCS |
| 225 | | SGDSVSSNSAAWNWIRQSPSRGLE | | GDALPKKYAYWYQQKSGQAPVLV |
| | | WLGRTYYRSKWYNDYAVSVKSRIT | | IYEDSKRPSGIPERFSGSSSGTM |
| | | INPDTSKNQFSLQLNSVTPEDTAV | | ATLTISGAQVEDEADYYCYSTDS |
| | | YYCASSGSYSDAFDIWGQGTMVTV | | SGNHRVFGGGTKLTVL |
| | | SS | | |
| clonotype | 630 | QVQLVQSGSELKKPGASVMVSCKA | 759 | QSVLTQPPSASGAPGQRVTMSCS |
| 226 | | SGYTFTRNGINWLRQAPGQGLEWM | | GSSSNIERTAVNWYSHLPGAAPK |
| | | GWIDTHTGNPTYVQGFTGRFVESL | | LLIYSNDQRPLGVPDRFAGSKSG |
| | | DTSVNTAYLQISSLRAEDTAVYYC | | SSASLAISGLQSEDEAAYFCAAW |
| | | AKDRTGYYHYYYFMDVWGKGTAVT | | DDSLNGWLFGGGTKLTVL |
| | | VSS | | |
| clonotype | 631 | QVQLVQSGTEMKKPGASVKVSCKA | 760 | QSVLTQPPSVSGAPGQRVTISCT |
| 228 | | SGYTFTTYGISWVRQAPGQGLEWM | | GNSSNIGADYDVQWYQQFPGTAP |
| | | GWISAYNGNTNYAQKLQARVTMTT | | KLLIYANIIRPSGVPDRESGSKS |
| | | DTSTNTAYMELRSLRSDDTAVYYC | | GTSASLAITGLQAEDEADYYCQS |
| | | ARSGYNWKYDYYYMDVWGKGTTVT | | YDSSLSGSLVFGGGTKLTVL |
| | | VSS | | |
| clonotype | 632 | EVQLVESGGGLVQPGGSLRLSCAA | 761 | SYELTQPPSVSVSPGQTARITCS |
| 230 | | SGFTFSSYWMSWVRQAPGKGLEWV | | GDALPKKYAYWYQQKSGQAPVLV |
| | | ANIKQDGSEKYYVDSVKGRFTISR | | IYEDSKRPSGIPERFSGSSSGTM |
| | | DNAKNSLYLQMNSLRAEDTAVYYC | | ATLTISGAQVEDEADYYCYSTDS |
| | | AREGGYDFWSGLNWFDPWGQGTLV | | SGNHRVFGGGTKLTVL |
| | | TVSS | | |
| clonotype | 633 | QVQLVQSGAEVKKPGASVKVSCKA | 762 | SYELTQPPSVSVSPGQTARITCS |
| 232 | | SGYTFTSYGISWVRQAPGQGLEWM | | GDALPKKYAYWYQQKSGQAPVLV |
| | | GWISAYNGNTNYAQKLQGRVTMTT | | IYEDSKRPSGIPERFSGSSSGTM |
| | | DTSTSTAYMELRSLRSDDTAVYYC | | ATLTISGAQVEDEADYYCYSTDS |
| | | ARAGGIAAAGTGYWFDPWGQGTLV | | SGNHRVFGGGTKLTVL |
| | | TVSS | | |
| clonotype | 634 | EVQLVESGGGLVKPGGSLRLSCAA | 763 | SSEMTQDPAVSVALGQTVRITCQ |
| 234 | | SGFTFSNAWMSWVRQAPGKGLEWV | | GDSLRSYYASWYQQKPGQAPVLV |
| | | GRIKSKTDGGTTDYAAPVKGRFTI | | IYGKNNRPSGIPDRFSGSSSGNT |
| | | SRDDSKNTLYLQMNSLKTEDTAVY | | ASLTITGAQAEDEADYYCNSRDS |
| | | YCTTADYDFWSGYYMDVWGKGTTV | | SGNHWVFGGGTKLTVL |
| | | TVSS | | |
| clonotype | 635 | QVQLQQSGPGLVKPSQTLSLTCAI | 764 | SYELTQPPSVSVSPGQTARITCS |
| 236 | | SGDSVSSNSAAWNWIRQSPSRGLE | | GDALPKKYAYWYQQKSGQAPVLV |
| | | WLGRTYYRSKWYNDYAVSVKSRIT | | IYEDSKRPSGIPERFSGSSSGTM |
| | | INPDTSKNQFSLQLNSVTPEDTAV | | ATLTISGAQVEDEADYYCYSTDS |
| | | YYCARDLELRGGAFDIWGQGTMVT | | SGNHRVFGGGTKLTVL |
| | | VSS | | |
| clonotype | 636 | EVHLVESGGGLVRPGGSLRLSCEV | 765 | SYELTQPPSVSVSPGQTARITCS |
| 242 | | SGFTFSTYSMNWVRQAPGKGLEWV | | GDALPKKYAYWYQQKSGQAPVLV |
| | | SSISSRSSYIYYADSVKGRFTISR | | IYEDSKRPSGIPERFSGSSSGTM |
| | | DNAKNSLYLQMNSLRAEDTAVYYC | | ATLTISGAQVEDEADYYCYSTDS |
| | | TRGEGATWGNYHCYYMDVWGKGTT | | SGNHRVFGGGTKLTVL |
| | | VIVSS | | |
| clonotype | 637 | QVQLVQSGAEVKKPGASVKVSCKA | 766 | SSELTQDPAVSVALGQTVRITCQ |
| 245 | | SGYSFTGYYMHWVRQAPGQGLEWM | | GDSLRSYYASWYQQKPGQAPVLV |
| | | GWINPNSGGTNYAQKFQGRVTMTW | | IYGKNNRPSGIPDRFSGSSSGNT |
| | | DTSISTAYMELSRLRSDDTAVYYC | | ASLTITGAQAEDEADYYCNSRDS |
| | | ARDQITMVRGFLGDWFDPWGQGTL | | SGNHLVFGGGTKLTVL |
| | | VTVSS | | |
| clonotype | 638 | QLQLQESGPGLVKPSETLSLTCTV | 767 | SYELTQPPSVSVSPGQTARITCS |
| 247 | | SGGSISSSSYYWGWIRQPPGKGLE | | GDALPKKYAYWYQQKSGQAPVLV |
| | | WIGSIYYSGSTYYNPSLKSRVTIS | | IYEDSKRPSGIPERFSGSSSGTM |
| | | VDTSKNQFSLKLSSVTAADTAVYY | | ATLTISGAQVEDEADYYCYSTDS |
| | | CARGYSYEFDYWGQGTLVTVSS | | SGNHRVFGGGTKLTVL |
| clonotype | 639 | QVQLQQSGPGLVKPSQTLSLTCAI | 768 | SYVLTQPPSVSVAPGKTARITCG |
| 248 | | SGDSVSSNSAAWNWIRQSPSRGLE | | GNNIGSKSVHWYQQKPGQAPVLV |
| | | WLGRTYYRSKWYNDYAVSVKSRIT | | IYYDSDRPSGIPERFSGSNSGNT |
| | | INPDTSKNQFSLQLNSVTPEDTAV | | ATLTISRVEAGDEADYYCQVWDS |
| | | YYCAREEIVGATTAFDIWGQGTMV | | SSDHWVFGGGTKLTVL |
| | | TVSS | | |
| clonotype | 640 | QVQLQQSGPGLVKPSQTLSLTCAI | 769 | SYELTQPPSVSVSPGQTARITCS |
| 249 | | SGDSVSSNSAAWNWIRQSPSRGLE | | GDALPKKYAYWYQQKSGQAPVLV |
| | | WLGRTYYRSKWYNDYAVSVKSRIT | | IYEDSKRPSGIPERFSGSSSGTM |
| | | INPDTSKNQFSLQLNSVTPEDTAV | | ATLTISGAQVEDEADYYCYSTDS |
| | | YYCARDYGGNSGWYFDLWGRGTLV | | SGNHRVFGGGTKLTVL |
| | | TVSS | | |
| clonotype | 641 | QVQLQQWGAGLLKPSETLSLTCAV | 770 | QSALTQPASVSGSPGQSITISCT |
| 251 | | YGGSFSGHYWNWIRQPPGKGLEWI | | GTSSDVGVYNYVSWYQQHPTKAP |
| | | GEINHSGFTNYNPSLKSRVTISVD | | KLMIYEVSNRPSGVSNRFSGSKS |
| | | TPKNQFSLNLSSVTAADTAVYYCA | | GNTASLTISGLQAEDEADYYCSS |
| | | REGLTGHVFDIWGQGTMVTVSS | | YTSSITWVFGGGTKLTVL |
| clonotype | 642 | QVQVQQSGPGLVKPSQTLSLTCAI | 771 | QSALTQPASVSGSPGQSITISCT |
| 252 | | SGDSVSSNSAAWNWIRQSPSRGLE | | GTSSDVGGYNYVSWYQQHPGKAP |
| | | WLGRTYYRSKWYNDFAVSVKSRIT | | KLMIYEVSNRPSGVSNRFSGSKS |
| | | INPDTSKNQFSLQLNSVTPEDTAV | | GNTASLTISGLQAEDEADYYCSS |
| | | YYCARGGGSGSYDWFDPWGQGTLV | | YTSSSTWVFGGGTKLTVL |
| | | TVSS | | |
| clonotype | 643 | EVRLVESGGGLVKPGGSLRLSCAA | 772 | SSELTQDPAVSVALGQTVRITCQ |
| 255 | | SGFIFSSYSMTWVRQAPGKGLEWV | | GDSLRSYYASWYQQKPGQAPVLV |
| | | SSISGSSSFVKYGDSVKGRFTISR | | IYGKNNRPSGIPDRFSGSSSGNT |
| | | DNAKNSLYLQMNSLRAEDTAVYYC | | ASLTITGAQAEDEADYYCNSRDS |
| | | AREGVLCSGGSCYREIFDYWGQVT | | SGNHLVFGGGTKLTVL |
| | | LVTVSS | | |
| clonotype | 644 | EVQLVESGEGLVKPGGSLRLSCVA | 773 | QSVLTQPPSVSGAPGQRVTISCT |
| 261 | | SGFDFTNAWMSWVRQAPGRGLEWV | | GSSSNIGAGYAVHWYQQFPGIAP |
| | | GRIKSKTDGGSIDYAAPVKGRFTI | | KLLIYGNINRPSGVPDRFSGSKS |
| | | SRDDSKTTLYLQMTSLRTEDTAVY | | DTSASLAITGLQAEDEADYYCQS |
| | | YCSTSPYYDFWSGYYGYIDYWGQG | | FDSSLSGVMFGGGTKLTVL |
| | | TLVTVSS | | |
| clonotype | 645 | EVHLVESGGGLVKPGGSLRLSCVA | 774 | WAQSVLTQPPSVSGAPGQRVTIS |
| 262 | | SRFTFSSAWMTWVRQVPGKGLEWI | | CSGSSSNIGAGYAVHWYQLLPGT |
| | | GRIKTKTEGGTTEYAAPVKGRFAI | | VPKLLIYGNLNRPSGVPDRESGS |
| | | SRDDSKKTLYLQMNSLKTEDTAVY | | MSDTSVSLAITGLQAEDEADYYC |
| | | YCSTSPYFDFWSGYYGYLDYWGQG | | QSYDSSLSGVVFGGGTKVTVL |
| | | TLVTVSS | | |
| clonotype | 646 | QLQLQESGPGLVKPSETLSLTCTV | 775 | QAVLTQPASLSASPGASASLTCT |
| 263 | | SGGSISSSSYYWGWIRQPPGKGLE | | LRSGINVGTYRIYWYQQKPGSPP |
| | | WIGSIYYSGSTYYNPSLKSRVTIS | | QYLLRYKSDSDKQQGSGVPSRES |
| | | VDTSKNQFSLKLSSVTAADTAVYY | | GSKDASANAGILLISGLQSEDEA |
| | | CARHAAAGGWFDPWGQGTLVTVSS | | DYYCMIWHSSAVVFGGGTKLTVL |
| clonotype | 647 | QLQLQESGPGLVKPSETLSLTCTV | 776 | SYELTQPPSVSVSPGQTASITCS |
| 264 | | SGGSISSSSYYWGWIRQPPGKGLE | | GDKLGDKYACWYQQKPGQSPVLV |
| | | WIGSIYYSGSTYYNPSLKSRVTIS | | IYQDSKRPSGIPERFSGSNSGNT |
| | | VDTSKNQFSLKLSSVTAADTAVYY | | ATLTISGTQAMDEADYYCQAWDS |
| | | CARRSSSGIGAFDIWGQGTMVTVS | | STVVFGGGTKLTVL |
| | | S | | |
| clonotype | 648 | QVQLQQWGAGLLKPSETLSLTCAV | 777 | SYVLTQPPSVSVAPGKTARITCG |
| 266 | | YGGSFSGYYWSWIRQPPGKGLEWI | | GNNIGSKSVHWYQQKPGQAPVLV |
| | | GEINHSGSTNYNPSLKSRVTISVD | | IYYDSDRPSGIPERFSGSNSGNT |
| | | TSKNQFSLKLSSVTAADTAVYYCA | | ATLTISRVEAGDEADYYCQVWDS |
| | | RGRGIAARPPYFDYWGQGTLVTVS | | SSDHVVFGGGTKLTVL |
| | | S | | |
| clonotype | 649 | QLQLQESGPGLVKPSETLSLTCTV | 778 | SYELTQPPSVSVSPGQTARITCS |
| 269 | | SGGSISSSSYYWGWIRQPPGKGLE | | GDALPKKYAYWYQQKSGQAPVLV |
| | | WIGSIYYSGSTYYNPSLKSRVTIS | | IYEDSKRPSGIPERFSGSSSGTM |
| | | VDTSKNQFSLKLSSVTAADTAVYY | | ATLTISGAQVEDEADYYCYSTDS |
| | | CASEYSSSSLDAFDIWGQGTMVTV | | SGNHRVFGGGTKLTVL |
| | | SS | | |
| clonotype | 650 | QVQLQQWGAGLLKPSETLSLTCAV | 779 | SYELTQPPSVSVSPGQTASITCS |
| 270 | | YGGSFSGYYWSWIRQPPGKGLEWI | | GDKLGDKYACWYQQKPGQSPVLV |
| | | GEINHSGSTNYNPSLKSRVTISVD | | IYQDSKRPSGIPERFSGSNSGNT |
| | | TSKNQFSLKLSSVTAADTAVYYCA | | ATLTISGTQAMDEADYYCQAWDS |
| | | RGTTVVTPTEYYYMDVWGKGTTVT | | STVVFGGGTKLTVL |
| | | VSS | | |
| clonotype | 651 | QVQLVQSGAEVKKPGASVKVSCKA | 780 | QSVLTQPPSVSGAPGQRVTISCT |
| 272 | | SGYTFTSYDINWVRQATGQGLEWM | | GSSSNIGAGYDVHWYQQLPGTAP |
| | | GWLNPKNGYTGYSHKFQGRVTMTR | | KLLIYGNSNRPSGVPDRESGSKS |
| | | NTSISTAYMELSSLRSEDAAVYFC | | GTSASLAITGLQAEDEADYYCQS |
| | | ARRGDFWSGYYSTSQNIVIHWEDS | | YDSSLSGSVFGGGTKLTVL |
| | | WGLGTLVTVSS | | |

ABLE 7
TVH-CDR and VL-CDR Sequences of Antibodies of Table 6

| clono | SEQ | HCD | SEQ | HCD | SEQ | | SEQ | LCD | SEQ | LCD | SEQ | LCD |
|---|---|---|---|---|---|---|---|---|---|---|---|---|
| type | ID | R1 | ID | R2 | ID | HCDR3 | ID | R1 | ID | R2 | ID | R3 |
| _id | NO | AA | NO | AA | NO | AA | NO | AA | NO | AA | NO | AA |
| clono | 2215 | GGSI | 2344 | YYSG | 2473 | CASL | 2602 | TGTS | 2731 | EVSN | 2860 | CSSY |
| type8 | | SSSS | | S | | TGDR | | SDVG | | RPS | | TSSS |
| | | Y | | | | FDYW | | GYNY | | | | TVVF |
| | | | | | | | | VS | | | | |
| clono | 2216 | GFTF | 2345 | KSKT | 2474 | CTGR | 2603 | SGDA | 2732 | EDSK | 2861 | CYST |
| type11 | | SNA | | DGGT | | PSIA | | LPKK | | RPS | | DSSG |
| | | | | | | ARHF | | YAY | | | | NHSV |
| | | | | | | DYW | | | | | | E |
| clono | 2217 | GFTF | 2346 | NWNG | 2475 | CARE | 2604 | TGSS | 2733 | GNSN | 2862 | CQSY |
| type14 | | DDY | | GS | | RLTI | | SNIG | | RPS | | DSSL |
| | | | | | | FGVV | | AGYD | | | | SGWV |
| | | | | | | NYYM | | VH | | | | F |
| | | | | | | DVW | | | | | | |
| clono | 2218 | GFTF | 2347 | SSSS | 2476 | CARD | 2605 | SGDV | 2734 | KDSE | 2863 | CYSA |
| type15 | | SSY | | ST | | GWDY | | LAKK | | RPS | | ADNN |
| | | | | | | W | | YAR | | | | RVF |
| clono | 2219 | GFTF | 2348 | YTTG | 2477 | CARG | 2606 | SGDA | 2735 | EDSK | 2864 | CYST |
| type16 | | SSS | | D | | YSGS | | LPKK | | RPS | | DSSG |
| | | | | | | YYGD | | YAY | | | | NRVF |
| | | | | | | FDYW | | | | | | |
| clono | 2220 | GFTF | 2349 | SGSG | 2478 | CAKK | 2607 | TGSS | 2736 | GNSN | 2865 | CQSY |
| type17 | | SSY | | GS | | PPRD | | SNIG | | RPS | | DSSL |
| | | | | | | SAFD | | AGYD | | | | SGSV |
| | | | | | | YW | | VH | | | | F |
| clono | 2221 | GYTF | 2350 | SAYN | 2479 | CARE | 2608 | TGTS | 2737 | EVSK | 2866 | CSSY |
| type25 | | TSY | | GN | | NSGS | | SDVG | | RPS | | AGSN |
| | | | | | | YYWF | | GYNY | | | | NVVF |
| | | | | | | DPW | | VS | | | | |
| clono | 2222 | GFTF | 2351 | SGSG | 2480 | CAKL | 2609 | TGTS | 2738 | DVSK | 2867 | CCSY |
| type27 | | SSY | | GS | | EYSS | | SDVG | | RPS | | AGSS |
| | | | | | | PDYW | | GYNY | | | | TLVF |
| | | | | | | | | VS | | | | |
| clono | 2223 | GGSF | 2352 | NHSG | 2481 | CARE | 2610 | QGDS | 2739 | GKNN | 2868 | CNSR |
| type36 | | SGY | | S | | GLLV | | LRSY | | RPS | | DSSG |
| | | | | | | GATL | | YAS | | | | NHLV |
| | | | | | | DAFD | | | | | | F |
| | | | | | | IW | | | | | | |
| clono | 2224 | GFTF | 2353 | WYDG | 2482 | CARD | 2611 | GGNN | 2740 | YDSD | 2869 | CQVW |
| type37 | | SSY | | SN | | TGIT | | IGSK | | RPS | | DSSS |
| | | | | | | MVRG | | SVH | | | | DHPV |
| | | | | | | VFDY | | | | | | F |
| | | | | | | W | | | | | | |
| clono | 2225 | GYTF | 2354 | SAYN | 2483 | CARD | 2612 | SGSS | 2741 | SNNQ | 2870 | CAAW |
| type44 | | TSY | | GN | | RTGI | | SNIG | | RPS | | DDSL |
| | | | | | | SAAG | | SNTV | | | | NGPV |
| | | | | | | PSNW | | N | | | | F |
| | | | | | | FDPW | | | | | | |
| clono | 2226 | GGSF | 2355 | NHSG | 2484 | CART | 2613 | TGTS | 2742 | EVSK | 2871 | CSSY |
| type45 | | SGY | | S | | SLAA | | SDVG | | RPS | | AGSN |
| | | | | | | ADFD | | GYNY | | | | NYVF |
| | | | | | | YW | | VS | | | | |
| clono | 2227 | GFTV | 2356 | YSGG | 2485 | CARA | 2614 | SGSS | 2743 | YDDL | 2872 | CAAW |
| type47 | | SSN | | S | | PDYY | | SNIG | | LPS | | DDRL |
| | | | | | | GSGS | | NNAV | | | | NGPV |
| | | | | | | LFDY | | N | | | | F |
| | | | | | | W | | | | | | |
| clono | 2228 | GFTF | 2357 | SGSS | 2486 | CASH | 2615 | GGNN | 2744 | RDSN | 2873 | CQVW |
| type52 | | STY | | SY | | WGHF | | IGSK | | RPS | | DSST |
| | | | | | | DYW | | NVH | | | | VVF |
| clono | 2229 | GFTF | 2358 | GTAG | 2487 | CARD | 2616 | SGDA | 2745 | EDSK | 2874 | CYST |
| type115 | | SSY | | D | | RTLD | | LPKK | | RPS | | DSSG |
| | | | | | | YW | | YAY | | | | NHWV |
| | | | | | | | | | | | | F |
| clono | 2230 | GYTF | 2359 | SAYN | 2488 | CARQ | 2617 | TGTS | 2746 | EVIK | 2875 | CSAY |
| type116 | | TTY | | GN | | IGDY | | SDVG | | RPS | | AGSN |
| | | | | | | W | | GYNY | | | | NVVF |
| | | | | | | | | VS | | | | |
| clono | 2231 | GFTF | 2360 | RSKA | 2489 | CTGP | 2618 | SGDK | 2747 | QDSK | 2876 | CQAW |
| type118 | | SVS | | NSYA | | FDNW | | LGDK | | RPS | | DSST |
| | | | | | | | | YAC | | | | GVF |
| clono | 2232 | GFTF | 2361 | KSKT | 2490 | CTTG | 2619 | SADA | 2748 | KDSE | 2877 | CQSA |
| type119 | | SNA | | DGGT | | GHYW | | LPNQ | | RPS | | DSSG |
| | | | | | | | | YAY | | | | TWVF |
| clono | 2233 | GFTF | 2362 | SWNS | 2491 | CAKA | 2620 | SGDA | 2749 | EDSK | 2878 | CYST |
| type122 | | DDY | | GS | | RSFD | | LPKK | | RPS | | DSSG |
| | | | | | | IW | | YAY | | | | NHRV |
| | | | | | | | | | | | | F |
| clono | 2234 | GFTF | 2363 | KSKT | 2492 | CRAD | 2621 | SGDA | 2750 | EDSK | 2879 | CYSI |
| type123 | | SDA | | DGGT | | MDVW | | LPKK | | RPS | | DSSG |
| | | | | | | | | YAY | | | | NHRV |
| | | | | | | | | | | | | F |
| clono | 2235 | GFTF | 2364 | NWNG | 2493 | CARD | 2622 | SGDA | 2751 | EDSK | 2880 | CYST |
| type124 | | DDY | | GS | | RGFD | | LPKK | | RPS | | DSSG |
| | | | | | | YW | | YAY | | | | NHRV |
| | | | | | | | | | | | | F |
| clono | 2236 | GFTV | 2365 | YSGG | 2494 | CARG | 2623 | SGDA | 2752 | EDSK | 2881 | CYST |
| type125 | | SSN | | S | | GDYF | | LPKK | | RPS | | DSSG |
| | | | | | | DYW | | YAY | | | | NHRV |
| | | | | | | | | | | | | F |
| clono | 2237 | GYTF | 2366 | SAYN | 2495 | CARG | 2624 | TGTS | 2753 | EVSD | 2882 | CSSY |
| type126 | | TSF | | DN | | ASFD | | SDVG | | RPS | | TRSS |
| | | | | | | FW | | GYNY | | | | TCVF |
| | | | | | | | | VS | | | | |
| clono | 2238 | GFTF | 2367 | RSKA | 2496 | CTGP | 2625 | TGTS | 2754 | EVSN | 2883 | CSSY |
| type127 | | SGS | | NSYA | | FDYW | | SDVG | | RPS | | TSSS |
| | | | | | | | | GYNY | | | | TWVE |
| | | | | | | | | VS | | | | |
| clono | 2239 | GFTV | 2368 | YSGG | 2497 | CARS | 2626 | TGTS | 2755 | DVSK | 2884 | CCSY |
| type128 | | SSN | | S | | FDAF | | SDVG | | RPS | | AGSS |
| | | | | | | DIW | | GYNY | | | | TFVV |
| | | | | | | | | VS | | | | F |
| clono | 2240 | GFTF | 2369 | SSSS | 2498 | CAGL | 2627 | SGDV | 2756 | KDSE | 2885 | CYSA |
| type130 | | SSY | | SY | | TGEL | | LAKK | | RPS | | ADNN |
| | | | | | | DYW | | YAR | | | | LVF |
| clono | 2241 | GFTF | 2370 | GTAG | 2499 | CARW | 2628 | SADA | 2757 | KDSE | 2886 | CQSA |
| type132 | | SSY | | D | | GTGG | | LPKQ | | RPS | | DSSG |
| | | | | | | FDYW | | YAY | | | | TWVF |
| clono | 2242 | GFTF | 2371 | SSSS | 2500 | CARR | 2629 | SGDA | 2758 | EDSK | 2887 | CYST |
| type133 | | SSY | | SY | | EGFF | | LPKK | | RPS | | DSSG |
| | | | | | | DYW | | YAY | | | | NHRV |
| | | | | | | | | | | | | F |
| clono | 2243 | GFTF | 2372 | KQDG | 2501 | CARD | 2630 | SGDA | 2759 | EDSK | 2888 | CYST |
| type134 | | SSY | | SE | | QLAP | | LPKK | | RPS | | DSSG |
| | | | | | | DYW | | YAY | | | | NHRV |
| | | | | | | | | | | | | F |
| clono | 2244 | GFTF | 2373 | SGSG | 2502 | CAKD | 2631 | SGDA | 2760 | EDSK | 2889 | CYST |
| type135 | | SSY | | GS | | SSGF | | LPKK | | RPS | | DSSG |
| | | | | | | DYW | | YAY | | | | NHRV |
| | | | | | | | | | | | | F |
| clono | 2245 | GFTF | 2374 | SGSG | 2503 | CAKD | 2632 | SGDA | 2761 | EDSK | 2890 | CYST |
| type136 | | SSY | | GS | | PQFF | | LPKK | | RPS | | DSSG |
| | | | | | | DYW | | YAY | | | | NHRV |
| | | | | | | | | | | | | F |
| clono | 2246 | GFTF | 2375 | SGSG | 2504 | CAKD | 2633 | SGDA | 2762 | EDSK | 2891 | CYST |
| type137 | | SSY | | GS | | GTAF | | LPKK | | RPS | | DSSG |
| | | | | | | DIW | | YAY | | | | NHRV |
| | | | | | | | | | | | | F |
| clono | 2247 | GFTF | 2376 | WYDG | 2505 | CARD | 2634 | GGNN | 2763 | RDSN | 2892 | CQVW |
| type138 | | SSY | | SN | | RGWG | | IGSK | | RPS | | DSST |
| | | | | | | LDYW | | NVH | | | | GVF |
| clono | 2248 | GFPF | 2377 | SYDG | 2506 | CARG | 2635 | QGDS | 2764 | GKNN | 2893 | CNSR |
| type140 | | SNS | | NS | | ELGD | | LRSY | | RPS | | DSSG |
| | | | | | | FDYW | | YAS | | | | NHLV |
| | | | | | | | | | | | | F |
| clono | 2249 | GYTF | 2378 | NPNS | 2507 | CARV | 2636 | ASST | 2765 | STSN | 2894 | CLLY |
| type141 | | TGY | | GG | | LELY | | GAVT | | KHS | | YGGA |
| | | | | | | FDYW | | SGYY | | | | VVF |
| | | | | | | | | PN | | | | |
| clono | 2250 | GFTF | 2379 | KSKT | 2508 | CTTR | 2637 | SGSS | 2766 | SNNQ | 2895 | CAAW |
| type143 | | SNA | | DGGT | | SDFQ | | SNIG | | RPS | | DDSL |
| | | | | | | HW | | SNTV | | | | NGWV |
| | | | | | | | | N | | | | F |
| clono | 2251 | GYTF | 2380 | NPNS | 2509 | CARD | 2638 | SGDK | 2767 | QDSK | 2896 | CQAW |
| type145 | | TSY | | GN | | QELR | | LGDK | | RPS | | DSST |
| | | | | | | VFDY | | YAC | | | | VVF |
| | | | | | | W | | | | | | |
| clono | 2252 | GFTF | 2381 | SYDG | 2510 | CAKA | 2639 | SGDK | 2768 | QDSK | 2897 | CQAW |
| type146 | | SSY | | SN | | SGYG | | LGDK | | RPS | | DSST |
| | | | | | | PFDY | | YAC | | | | VVF |
| | | | | | | W | | | | | | |
| clono | 2253 | GYSF | 2382 | YPGD | 2511 | CARH | 2640 | SGDK | 2769 | QDSK | 2898 | CQAW |
| type147 | | TSY | | SD | | SSSS | | LGDK | | RPS | | DSST |
| | | | | | | HFDY | | YAC | | | | VVF |
| | | | | | | W | | | | | | |
| clono | 2254 | GFTF | 2383 | SSSS | 2512 | CARD | 2641 | SGDA | 2770 | EDSK | 2899 | CYST |
| type148 | | SSY | | SY | | RGNS | | LPKK | | RPS | | DSSG |
| | | | | | | LFDY | | YAY | | | | NHRV |
| | | | | | | W | | | | | | F |
| clono | 2255 | GYTF | 2384 | NPNS | 2513 | CARD | 2642 | SGDA | 2771 | EDSK | 2900 | CYST |
| type150 | | TGY | | GG | | KSLE | | LPKK | | RPS | | DSSG |
| | | | | | | WFDY | | YAY | | | | NHRV |
| | | | | | | W | | | | | | F |
| clono | 2256 | GFTF | 2385 | GTAG | 2514 | CARG | 2643 | SGDA | 2772 | EDSK | 2901 | CYST |
| type151 | | SSY | | D | | DWNY | | LPKK | | RPS | | DSSG |
| | | | | | | GGFD | | YAY | | | | NHRV |
| | | | | | | YW | | | | | | F |
| clono | 2257 | GFTF | 2386 | KSKT | 2515 | CTTA | 2644 | SGDA | 2773 | EDSK | 2902 | CYST |
| type152 | | SNA | | DGGT | | PDAF | | LPKK | | RPS | | DSSG |
| | | | | | | DIW | | YAY | | | | NHRV |
| | | | | | | | | | | | | F |
| clono | 2258 | GFTF | 2387 | SGSG | 2516 | CASG | 2645 | SGDA | 2774 | EDSK | 2903 | CYST |
| type153 | | SSY | | GS | | ITGT | | LPKK | | RPS | | DSSG |
| | | | | | | TGDY | | YAY | | | | NHRV |
| | | | | | | W | | | | | | F |
| clono | 2259 | GFTF | 2388 | SGSG | 2517 | CAKE | 2646 | SGDA | 2775 | EDSK | 2904 | CYST |
| type154 | | SSY | | GS | | GAHD | | LPKK | | RPS | | DSSG |
| | | | | | | AFDI | | YAY | | | | NHRV |
| | | | | | | W | | | | | | F |
| clono | 2260 | GFTF | 2389 | SGSG | 2518 | CAKD | 2647 | SGDA | 2776 | EDSK | 2905 | CYST |
| type156 | | SSY | | GS | | KGEL | | LPKK | | RPS | | DSSG |
| | | | | | | PFDY | | YAY | | | | NHRV |
| | | | | | | W | | | | | | F |
| clono | 2261 | GFTF | 2390 | WYDG | 2519 | CAKL | 2648 | QGDS | 2777 | GKNN | 2906 | CNSR |
| type157 | | SSY | | SN | | GVRD | | LRSY | | RPS | | DSSG |
| | | | | | | YMDV | | YAS | | | | NHWV |
| | | | | | | W | | | | | | F |
| clono | 2262 | GFTF | 2391 | NWNG | 2520 | CARE | 2649 | QGDS | 2778 | GKNN | 2907 | CNSR |
| type158 | | DDY | | GS | | GGGW | | LRSY | | RPS | | DSSG |
| | | | | | | VFDY | | YAS | | | | NHWV |
| | | | | | | W | | | | | | F |
| clono | 2263 | GYSF | 2392 | YPGD | 2521 | CARG | 2650 | SGDA | 2779 | EDSK | 2908 | CYST |
| type159 | | TSY | | SD | | GGGD | | LPKK | | RPS | | DSSG |
| | | | | | | PFDY | | YAY | | | | NHRV |
| | | | | | | W | | | | | | F |
| clono | 2264 | GYTF | 2393 | NPNS | 2522 | CARP | 2651 | TGTS | 2780 | DVSN | 2909 | CSSY |
| type160 | | TGY | | GG | | YNWN | | SDVG | | RPS | | TTSS |
| | | | | | | SFDY | | GYNY | | | | TWVF |
| | | | | | | W | | VS | | | | |
| clono | 2265 | GFTF | 2394 | TWNS | 2523 | CAKD | 2652 | TGTS | 2781 | EVSN | 2910 | CNSY |
| type161 | | DDH | | NI | | NDWN | | SDVG | | RPS | | TTNT |
| | | | | | | GFDY | | GYNY | | | | TRVF |
| | | | | | | W | | VS | | | | |
| clono | 2266 | GFTF | 2395 | SWNS | 2524 | CAKD | 2653 | TGTS | 2782 | EVSN | 2911 | CSSY |
| type162 | | DDY | | GS | | NWNY | | SDVG | | RPS | | TSSS |
| | | | | | | AFDI | | GYNY | | | | TRVF |
| | | | | | | W | | VS | | | | |
| clono | 2267 | GFTF | 2396 | NSDG | 2525 | CARD | 2654 | TLRS | 2783 | YKSD | 2912 | CMTW |
| type164 | | SSY | | GN | | LDWT | | GIYV | | SDKQ | | HSSA |
| | | | | | | LFDY | | GTYR | | QGS | | VVF |
| | | | | | | W | | IY | | | | |
| clono | 2268 | GFTF | 2397 | KQDG | 2526 | CAGD | 2655 | SGDK | 2784 | QDSK | 2913 | CQAW |
| type165 | | SSY | | SE | | YSNY | | LGDK | | RPS | | DSST |
| | | | | | | GWFD | | YAC | | | | V |
| | | | | | | PW | | | | | | |
| clono | 2269 | GYTF | 2398 | NPNS | 2527 | CARA | 2656 | SGDK | 2785 | QDSK | 2914 | CQAW |
| type166 | | TSY | | GN | | RDSG | | LGDK | | RPS | | DSST |
| | | | | | | YYMD | | YAC | | | | VVF |
| | | | | | | VW | | | | | | |
| clono | 2270 | GFTF | 2399 | WYDG | 2528 | CARA | 2657 | SGDV | 2786 | KDSE | 2915 | CYSA |
| type167 | | SSY | | SN | | TAMV | | LAKK | | RPS | | ADNN |
| | | | | | | TGID | | YAR | | | | WVF |
| | | | | | | YW | | | | | | |
| clono | 2271 | GFTF | 2400 | RSKA | 2529 | CTGS | 2658 | SGDV | 2787 | KDSE | 2916 | CYSA |
| type168 | | SGS | | NSYA | | SGSY | | LAKK | | RPS | | ADNN |
| | | | | | | FDYW | | YAR | | | | LVF |
| clono | 2272 | GFTF | 2401 | SSSS | 2530 | CARS | 2659 | SGDA | 2788 | EDSK | 2917 | CYST |
| type169 | | SSY | | SY | | PYNW | | LPKK | | RPS | | DSSG |
| | | | | | | NYVD | | YAY | | | | NHRV |
| | | | | | | YW | | | | | | F |
| clono | 2273 | GYTL | 2402 | DPED | 2531 | CATE | 2660 | SGDA | 2789 | EDSK | 2918 | CYST |
| type170 | | TEL | | GE | | GPST | | LPKK | | RPS | | DSSG |
| | | | | | | FSFD | | YAY | | | | NHRV |
| | | | | | | YW | | | | | | F |
| clono | 2274 | GYTL | 2403 | DPED | 2532 | CATA | 2661 | QGDS | 2790 | GKNN | 2919 | CNSR |
| typel71 | | TEL | | GE | | NWND | | LRSY | | RPS | | DSSG |
| | | | | | | EAFD | | YAS | | | | NHLV |
| | | | | | | IW | | | | | | F |
| clono | 2275 | GFTF | 2404 | SSSS | 2533 | CARD | 2662 | SGDA | 2791 | EDSK | 2920 | CYST |
| type172 | | SSY | | ST | | ELTG | | LPKK | | RPS | | DSSG |
| | | | | | | DAFD | | YAY | | | | NHRV |
| | | | | | | IW | | | | | | F |
| clono | 2276 | GFTF | 2405 | KSKT | 2534 | CTTE | 2663 | SGDA | 2792 | EDSK | 2921 | CYST |
| type173 | | SNA | | DGGT | | ALGI | | LPKK | | RPS | | DSSG |
| | | | | | | FDYW | | YAY | | | | NHRV |
| | | | | | | | | | | | | F |
| clono | 2277 | GFTF | 2406 | SSSS | 2535 | CARD | 2664 | ASST | 2793 | STSN | 2922 | CLLY |
| type174 | | SSY | | SY | | GSSG | | GAVT | | KHS | | YGGA |
| | | | | | | FLFD | | SGYY | | | | WVF |
| | | | | | | YW | | PN | | | | |
| clono | 2278 | GFTF | 2407 | SGSG | 2536 | CAKH | 2665 | TGTS | 2794 | EVSK | 2923 | CSSY |
| type175 | | SSY | | GS | | YYDS | | SDVG | | RPS | | AGSN |
| | | | | | | RSFD | | GYNY | | | | NLVF |
| | | | | | | YW | | VS | | | | |
| clono | 2279 | GGSI | 2408 | NHSG | 2537 | CARD | 2666 | TGSS | 2795 | DNNN | 2924 | CQSY |
| type176 | | SSSD | | T | | FQGT | | SNIG | | RPS | | DGSL |
| | | | | | | GPFD | | AGYD | | | | NGWV |
| | | | | | | YW | | VH | | | | F |
| clono | 2280 | GGSF | 2409 | NHSG | 2538 | CARG | 2667 | QGDS | 2796 | GKNN | 2925 | CKSR |
| type178 | | SGY | | S | | RLYS | | LRNY | | RPS | | DRSG |
| | | | | | | GSFS | | YAS | | | | NHWV |
| | | | | | | FDYW | | | | | | F |
| clono | 2281 | GFTF | 2410 | KQDG | 2539 | CARD | 2668 | QGDS | 2797 | GKNN | 2926 | CNSR |
| type179 | | SSY | | SE | | GGYN | | LRSY | | RPS | | DSSG |
| | | | | | | WNFF | | YAS | | | | NHVV |
| | | | | | | DYW | | | | | | F |
| clono | 2282 | GFSL | 2411 | YWND | 2540 | CTHR | 2669 | QGDS | 2798 | GKNN | 2927 | CNSR |
| type180 | | STSG | | D | | DAAM | | LRSY | | RPS | | DSSG |
| | | V | | | | VYFD | | YAS | | | | NHWV |
| | | | | | | YW | | | | | | F |
| clono | 2283 | GYTF | 2412 | SAYN | 2541 | CARW | 2670 | SGDA | 2799 | EDSK | 2928 | CYST |
| type181 | | TSY | | GN | | YYGS | | LPKK | | RPS | | DSSG |
| | | | | | | GSYF | | YAY | | | | NHRV |
| | | | | | | DYW | | | | | | F |
| clono | 2284 | GFTF | 2413 | NTAG | 2542 | CARG | 2671 | QGDN | 2800 | GKNN | 2929 | CNSR |
| type182 | | SRY | | D | | WNYG | | LRNY | | RPS | | DISG |
| | | | | | | SGSC | | SVS | | | | KHWV |
| | | | | | | FDNW | | | | | | F |
| clono | 2285 | GFTF | 2414 | YTTG | 2543 | CARG | 2672 | SGDA | 2801 | EDSK | 2930 | CYST |
| type183 | | SSS | | D | | FSGT | | LPKK | | RPS | | DSSG |
| | | | | | | YYGD | | YAY | | | | NHWV |
| | | | | | | FDYW | | | | | | F |
| clono | 2286 | GFTF | 2415 | SSSS | 2544 | CARE | 2673 | SGDA | 2802 | EDSK | 2931 | CYST |
| type184 | | SSY | | ST | | GEWE | | LPKK | | RPS | | DSSG |
| | | | | | | PLHM | | YAY | | | | NHRV |
| | | | | | | DVW | | | | | | F |
| clono | 2287 | GFTF | 2416 | SGSG | 2545 | CAKS | 2674 | SGDA | 2803 | EDSK | 2932 | CYST |
| type185 | | SSY | | GS | | LSGS | | LPKK | | RPS | | DSSG |
| | | | | | | YVYM | | YAY | | | | NHRV |
| | | | | | | DVW | | | | | | F |
| clono | 2288 | GFTF | 2417 | SYDG | 2546 | CAKG | 2675 | QGDS | 2804 | GKNN | 2933 | CNSR |
| type186 | | SSY | | SN | | FLEW | | LRSY | | RPS | | DSSG |
| | | | | | | LLGF | | YAS | | | | NHWV |
| | | | | | | DYW | | | | | | F |
| clono | 2289 | GFTF | 2418 | SSSG | 2547 | CARD | 2676 | GGNN | 2805 | YDSD | 2934 | CQVW |
| type187 | | SDY | | ST | | GGSS | | IGSK | | RPS | | DSSS |
| | | | | | | GYYS | | SVH | | | | DHVV |
| | | | | | | DYW | | | | | | F |
| clono | 2290 | GFTF | 2419 | NSDG | 2548 | CTRD | 2677 | QGDS | 2806 | GKNN | 2935 | CNSR |
| type188 | | SEY | | SR | | LVYS | | LRSY | | RPS | | DSSG |
| | | | | | | SGWY | | YAN | | | | NHWV |
| | | | | | | DYW | | | | | | F |
| clono | 2291 | GFTF | 2420 | NSDG | 2549 | CARE | 2678 | QGDS | 2807 | GKNN | 2936 | CNSR |
| type189 | | SSY | | SG | | GIKA | | LRTY | | RPS | | DSSG |
| | | | | | | SDAF | | YAS | | | | SHVV |
| | | | | | | DIW | | | | | | F |
| clono | 2292 | GFTF | 2421 | SWNS | 2550 | CAKD | 2679 | SGDA | 2808 | EDSK | 2937 | CYST |
| type190 | | DDY | | GS | | IDPS | | LPKK | | RPS | | DSSG |
| | | | | | | ITGT | | YAY | | | | NHSV |
| | | | | | | DYW | | | | | | VF |
| clono | 2293 | GFTF | 2422 | SHSG | 2551 | CAGL | 2680 | ASST | 2809 | STSN | 2938 | CLLY |
| type191 | | SDY | | TT | | RHFD | | GAVT | | KHS | | YGGA |
| | | | | | | WLGF | | SGYY | | | | WVF |
| | | | | | | DSW | | PN | | | | |
| clono | 2294 | GFTF | 2423 | NDSG | 2552 | CAKE | 2681 | TGTS | 2810 | EVII | 2939 | CSSY |
| type192 | | SSY | | YS | | DNWN | | SDVG | | RPS | | TSSS |
| | | | | | | YGWF | | GYNY | | | | TWVF |
| | | | | | | DPW | | VS | | | | |
| clono | 2295 | GFTF | 2424 | GTAG | 2553 | CARE | 2682 | TGTS | 2811 | EVSN | 2940 | CSSY |
| type193 | | SSY | | D | | ETGT | | SDVG | | RPS | | TSSS |
| | | | | | | TSWY | | GYNY | | | | TLYV |
| | | | | | | FDIW | | VS | | | | F |
| clono | 2296 | GFTF | 2425 | SSSS | 2554 | CARG | 2683 | TGTS | 2812 | EVSN | 2941 | CSSY |
| type194 | | SSY | | ST | | YSYG | | SDVG | | RPS | | TSSS |
| | | | | | | YWYF | | GYNY | | | | TPYV |
| | | | | | | DLW | | VS | | | | F |
| clono | 2297 | GYTL | 2426 | DPED | 2555 | CATP | 2684 | SGDK | 2813 | QDSK | 2942 | CQAW |
| type195 | | TEL | | GE | | YCSG | | LGDK | | RPS | | DSST |
| | | | | | | GSCH | | YAC | | | | VVF |
| | | | | | | FDYW | | | | | | |
| clono | 2298 | GFTF | 2427 | SSSS | 2556 | CARD | 2685 | SGDA | 2814 | EDSK | 2943 | CYST |
| type196 | | SSY | | SY | | DYGG | | LPKK | | RPS | | DSSG |
| | | | | | | NSVY | | YAY | | | | NHRV |
| | | | | | | FDYW | | | | | | F |
| clono | 2299 | GFTF | 2428 | WSDG | 2557 | CVRA | 2686 | GGNN | 2815 | YDSD | 2944 | CQVW |
| type198 | | SNY | | SN | | ARYS | | IGSK | | RPS | | DSSS |
| | | | | | | GTYI | | SVH | | | | YHYV |
| | | | | | | FDYW | | | | | | F |
| clono | 2300 | GFTF | 2429 | KSKT | 2558 | CTTD | 2687 | SGDA | 2816 | EDSK | 2945 | CYST |
| type199 | | SNA | | DGGT | | PGYS | | LPKK | | RPS | | DSSG |
| | | | | | | YGVD | | YAY | | | | NHRV |
| | | | | | | YW | | | | | | F |
| clono | 2301 | GYSF | 2430 | YPGD | 2559 | CARP | 2688 | SGDA | 2817 | EDSK | 2946 | CYST |
| type200 | | TSY | | SD | | EYSS | | LPKK | | RPS | | DSSG |
| | | | | | | SSGY | | YAY | | | | NHRV |
| | | | | | | FQHW | | | | | | |
| clono | 2302 | GFTF | 2431 | KQDG | 2560 | CARE | 2689 | ASST | 2818 | STSN | 2947 | CLLY |
| type201 | | SSY | | SE | | YNWN | | GAVT | | KHS | | YGGA |
| | | | | | | YEDA | | SGYY | | | | QVF |
| | | | | | | FDIW | | PN | | | | |
| clono | 2303 | GFSL | 2432 | YWND | 2561 | CAHR | 2690 | TGTS | 2819 | DVSK | 2948 | CCSY |
| type202 | | STSG | | D | | RGSY | | SDVG | | RPS | | AGSS |
| | | V | | | | SNWF | | GYNY | | | | TWVF |
| | | | | | | DPW | | VS | | | | |
| clono | 2304 | GYTF | 2433 | SAYN | 2562 | CART | 2691 | SGSS | 2820 | HDVL | 2949 | CAAW |
| type203 | | SSY | | GN | | LFGV | | SNIG | | LSS | | DGRL |
| | | | | | | VKNW | | NNAV | | | | NEWV |
| | | | | | | FDPW | | N | | | | F |
| clono | 2305 | YMHW | 2434 | NYAQ | 2563 | CARE | 2692 | SGSS | 2821 | SNNQ | 2950 | CAAW |
| type204 | | VRQ | | KF | | VLGG | | SNIG | | RPS | | DDSL |
| | | | | | | GDCP | | SNTV | | | | NGVV |
| | | | | | | FDYW | | N | | | | F |
| clono | 2306 | GYTF | 2435 | NPNS | 2564 | CARS | 2693 | SGSI | 2822 | YDDL | 2951 | CTAW |
| type205 | | TDH | | GG | | DGGS | | SNIG | | LPS | | DDRL |
| | | | | | | HYVF | | NNAV | | | | NGPV |
| | | | | | | FDDW | | S | | | | F |
| clono | 2307 | GFTF | 2436 | SWNS | 2565 | CAKD | 2694 | TGTS | 2823 | EVSK | 2952 | CSSY |
| type206 | | DDY | | GS | | IAYS | | SDVG | | RPS | | AGSN |
| | | | | | | SSGH | | GYNY | | | | NLVF |
| | | | | | | FDYW | | VS | | | | |
| clono | 2308 | GGSI | 2437 | YHSG | 2566 | CARA | 2695 | SGSS | 2824 | GNRN | 2953 | CQSY |
| type207 | | TSSN | | N | | PLTG | | SNIG | | RPS | | DSSL |
| | | | | | | TTNW | | AGYD | | | | SGWV |
| | | | | | | FDPW | | VH | | | | F |
| clono | 2309 | GFTF | 2438 | SSSS | 2567 | CAGV | 2696 | SGDV | 2825 | KDSE | 2954 | CYSA |
| type209 | | SSY | | SY | | LYYD | | LAKK | | RPS | | ADNN |
| | | | | | | SSGY | | YAR | | | | LVF |
| | | | | | | PFDY | | | | | | |
| | | | | | | W | | | | | | |
| clono | 2310 | GYTF | 2439 | NTNT | 2568 | CARD | 2697 | SGDK | 2826 | QDSK | 2955 | CQAW |
| type210 | | TSY | | GN | | PLAA | | LGDK | | RPS | | DSST |
| | | | | | | RPVG | | YAC | | | | AVF |
| | | | | | | WFDP | | | | | | |
| | | | | | | W | | | | | | |
| clono | 2311 | GFTF | 2440 | SSSS | 2569 | CARE | 2698 | QGDS | 2827 | GKNN | 2956 | CNSR |
| type211 | | SSY | | SY | | DGYS | | LRSY | | RPS | | DSSG |
| | | | | | | SGWN | | YAS | | | | NHWV |
| | | | | | | YFDY | | | | | | F |
| | | | | | | W | | | | | | |
| clono | 2312 | GYTL | 2441 | DPED | 2570 | CATG | 2699 | GGNN | 2828 | YDSD | 2957 | CQVW |
| type212 | | TEL | | GE | | GQTI | | IGSK | | RPS | | DSSS |
| | | | | | | VAAR | | SVH | | | | DHVV |
| | | | | | | VFDY | | | | | | F |
| | | | | | | W | | | | | | |
| clono | 2313 | GYTV | 2442 | NTNT | 2571 | CARD | 2700 | QGDS | 2829 | GKNN | 2958 | CNSR |
| type213 | | TRH | | GT | | QTPS | | LRSY | | RPS | | DSSG |
| | | | | | | DHYY | | YAS | | | | NHYV |
| | | | | | | YMDV | | | | | | F |
| | | | | | | W | | | | | | |
| clono | 2314 | GYAF | 2443 | RPNS | 2572 | CARD | 2701 | QGDS | 2830 | GKNN | 2959 | CNSR |
| type214 | | RGQ | | GD | | RGIT | | LRSY | | RPS | | DSSG |
| | | | | | | MRLD | | YAS | | | | NHLV |
| | | | | | | NMDV | | | | | | F |
| | | | | | | W | | | | | | |
| clono | 2315 | GFTF | 2444 | RGKP | 2573 | CTRR | 2702 | TGTS | 2831 | DVSK | 2960 | CCSY |
| type215 | | SDS | | NTYA | | YNWN | | SDVG | | RPS | | AGSN |
| | | | | | | DVGF | | AYNY | | | | TYVF |
| | | | | | | DYW | | VS | | | | |
| clono | 2316 | GFSL | 2445 | YWND | 2574 | CAHR | 2703 | SGDK | 2832 | QDSK | 2961 | CQAW |
| type217 | | STSG | | D | | PGIT | | LGDK | | RPS | | DSST |
| | | V | | | | GNTG | | YAC | | | | VVF |
| | | | | | | YFDY | | | | | | |
| | | | | | | W | | | | | | |
| clono | 2317 | GYTF | 2446 | SAYN | 2575 | CARC | 2704 | SGDK | 2833 | QDSK | 2962 | CQAW |
| type218 | | TSY | | GN | | RYSG | | LGDK | | RPS | | DSST |
| | | | | | | SLTS | | YAC | | | | VVF |
| | | | | | | YYMD | | | | | | |
| | | | | | | VW | | | | | | |
| clono | 2318 | GFTF | 2447 | SWNS | 2576 | CAKD | 2705 | SGDK | 2834 | QDSK | 2963 | CQAW |
| type219 | | DDY | | GS | | MITG | | LGDK | | RPS | | DSST |
| | | | | | | TTNY | | YAC | | | | VVF |
| | | | | | | YYMD | | | | | | |
| | | | | | | VW | | | | | | |
| clono | 2319 | GFTF | 2448 | SRNS | 2577 | CAKG | 2706 | GENN | 2835 | RDGN | 2964 | CQVW |
| type220 | | DDY | | GS | | GYDF | | IVNK | | RPS | | DNNT |
| | | | | | | WSGY | | NVH | | | | PWVF |
| | | | | | | YPFD | | | | | | |
| | | | | | | PW | | | | | | |
| clono | 2320 | GFTF | 2449 | KSKT | 2578 | CTTE | 2707 | SGDA | 2836 | EDSK | 2965 | CYST |
| type223 | | SNA | | DGGT | | GTTV | | LPKK | | RPS | | DSSG |
| | | | | | | TTWA | | YAY | | | | NHRV |
| | | | | | | FDIW | | | | | | F |
| clono | 2321 | GDSV | 2450 | YYRS | 2579 | CASS | 2708 | SGDA | 2837 | EDSK | 2966 | CYST |
| type225 | | SSNS | | KWY | | GSYS | | LPKK | | RPS | | DSSG |
| | | A | | | | DAFD | | YAY | | | | NHRV |
| | | | | | | IW | | | | | | F |
| clono | 2322 | GYTF | 2451 | DTHT | 2580 | CAKD | 2709 | SGSS | 2838 | SNDQ | 2967 | CAAW |
| type226 | | TRN | | GN | | RTGY | | SNIE | | RPL | | DDSL |
| | | | | | | YHYY | | RTAV | | | | NGWL |
| | | | | | | YFMD | | N | | | | F |
| | | | | | | VW | | | | | | |
| clono | 2323 | GYTF | 2452 | SAYN | 2581 | CARS | 2710 | TGNS | 2839 | ANII | 2968 | CQSY |
| type228 | | TTY | | GN | | GYNW | | SNIG | | RPS | | DSSL |
| | | | | | | KYDY | | ADYD | | | | SGSL |
| | | | | | | YYMD | | VQ | | | | VF |
| | | | | | | VW | | | | | | |
| clono | 2324 | GFTF | 2453 | KQDG | 2582 | CARE | 2711 | SGDA | 2840 | EDSK | 2969 | CYST |
| type230 | | SSY | | SE | | GGYD | | LPKK | | RPS | | DSSG |
| | | | | | | FWSG | | YAY | | | | NHRV |
| | | | | | | LNWF | | | | | | F |
| | | | | | | DPW | | | | | | |
| clono | 2325 | GYTF | 2454 | SAYN | 2583 | CARA | 2712 | SGDA | 2841 | EDSK | 2970 | CYST |
| type232 | | TSY | | GN | | GGIA | | LPKK | | RPS | | DSSG |
| | | | | | | AAGT | | YAY | | | | NHRV |
| | | | | | | GYWF | | | | | | F |
| | | | | | | DPW | | | | | | |
| clono | 2326 | GFTF | 2455 | KSKT | 2584 | CTTA | 2713 | QGDS | 2842 | GKNN | 2971 | CNSR |
| type234 | | SNA | | DGGT | | DYDF | | LRSY | | RPS | | DSSG |
| | | | | | | WSGY | | YAS | | | | NHWV |
| | | | | | | YMDV | | | | | | F |
| | | | | | | W | | | | | | |
| clono | 2327 | GDSV | 2456 | YYRS | 2585 | CARD | 2714 | SGDA | 2843 | EDSK | 2972 | CYST |
| type236 | | SSNS | | KWY | | LELR | | LPKK | | RPS | | DSSG |
| | | A | | | | GGAF | | YAY | | | | NHRV |
| | | | | | | DIW | | | | | | F |
| clono | 2328 | GFTF | 2457 | SSRS | 2586 | CTRG | 2715 | SGDA | 2844 | EDSK | 2973 | CYST |
| type242 | | STY | | SY | | EGAT | | LPKK | | RPS | | DSSG |
| | | | | | | WGNY | | YAY | | | | NHRV |
| | | | | | | HCYY | | | | | | F |
| | | | | | | MDVW | | | | | | |
| clono | 2329 | GYSF | 2458 | NPNS | 2587 | CARD | 2716 | QGDS | 2845 | GKNN | 2974 | CNSR |
| type245 | | TGY | | GG | | QITM | | LRSY | | RPS | | DSSG |
| | | | | | | VRGF | | YAS | | | | NHLV |
| | | | | | | LGDW | | | | | | F |
| | | | | | | FDPW | | | | | | |
| clono | 2330 | GGSI | 2459 | YYSG | 2588 | CARG | 2717 | SGDA | 2846 | EDSK | 2975 | CYST |
| type247 | | SSSS | | S | | YSYE | | LPKK | | RPS | | DSSG |
| | | Y | | | | FDYW | | YAY | | | | NHRV |
| | | | | | | | | | | | | F |
| clono | 2331 | GDSV | 2460 | YYRS | 2589 | CARE | 2718 | GGNN | 2847 | YDSD | 2976 | CQVW |
| type248 | | SSNS | | KWY | | EIVG | | IGSK | | RPS | | DSSS |
| | | A | | | | ATTA | | SVH | | | | DHWV |
| | | | | | | FDIW | | | | | | F |
| clono | 2332 | GDSV | 2461 | YYRS | 2590 | CARD | 2719 | SGDA | 2848 | EDSK | 2977 | CYST |
| type249 | | SSNS | | KWY | | YGGN | | LPKK | | RPS | | DSSG |
| | | A | | | | SGWY | | YAY | | | | NHRV |
| | | | | | | FDLW | | | | | | F |
| clono | 2333 | GGSF | 2462 | NHSG | 2591 | CARE | 2720 | TGTS | 2849 | EVSN | 2978 | CSSY |
| type251 | | SGH | | F | | GLTG | | SDVG | | RPS | | TSSI |
| | | | | | | HVFD | | VYNY | | | | TWVF |
| | | | | | | IW | | VS | | | | |
| clono | 2334 | GDSV | 2463 | YYRS | 2592 | CARG | 2721 | TGTS | 2850 | EVSN | 2979 | CSSY |
| type252 | | SSNS | | KWY | | GGSG | | SDVG | | RPS | | TSSS |
| | | A | | | | SYDW | | GYNY | | | | TWVF |
| | | | | | | FDPW | | VS | | | | |
| clono | 2335 | GFIF | 2464 | SGSS | 2593 | CARE | 2722 | QGDS | 2851 | GKNN | 2980 | CNSR |
| type255 | | SSY | | SF | | GVLC | | LRSY | | RPS | | DSSG |
| | | | | | | SGGS | | YAS | | | | NHLV |
| | | | | | | CYRE | | | | | | F |
| | | | | | | IFDY | | | | | | |
| | | | | | | W | | | | | | |
| clono | 2336 | GFDF | 2465 | KSKT | 2594 | CSTS | 2723 | TGSS | 2852 | GNIN | 2981 | CQSF |
| type261 | | TNA | | DGGS | | PYYD | | SNIG | | RPS | | DSSL |
| | | | | | | EWSG | | AGYA | | | | SGVM |
| | | | | | | YYGY | | VH | | | | F |
| | | | | | | IDYW | | | | | | |
| clono | 2337 | RFTF | 2466 | KTKT | 2595 | CSTS | 2724 | SCSG | 2853 | IYGN | 2982 | CQSY |
| type262 | | SSA | | EGGT | | PYFD | | SSSN | | LNR | | DSSL |
| | | | | | | FWSG | | IGAG | | | | SGVV |
| | | | | | | YYGY | | YA | | | | F |
| | | | | | | LDYW | | | | | | |
| clono | 2338 | GGSI | 2467 | YYSG | 2596 | CARH | 2725 | TLRS | 2854 | YKSD | 2983 | CMIW |
| type263 | | SSSS | | S | | AAAG | | GINV | | SDKQ | | HSSA |
| | | Y | | | | GWFD | | GTYR | | QGS | | VVF |
| | | | | | | PW | | IY | | | | |
| clono | 2339 | GGSI | 2468 | YYSG | 2597 | CARR | 2726 | SGDK | 2855 | QDSK | 2984 | CQAW |
| type264 | | SSSS | | S | | SSSG | | LGDK | | RPS | | DSST |
| | | Y | | | | IGAF | | YAC | | | | VVF |
| | | | | | | DIW | | | | | | |
| clono | 2340 | GGSF | 2469 | NHSG | 2598 | CARG | 2727 | GGNN | 2856 | YDSD | 2985 | CQVW |
| type266 | | SGY | | S | | RGIA | | IGSK | | RPS | | DSSS |
| | | | | | | ARPP | | SVH | | | | DHVV |
| | | | | | | YFDY | | | | | | F |
| | | | | | | W | | | | | | |
| clono | 2341 | GGSI | 2470 | YYSG | 2599 | CASE | 2728 | SGDA | 2857 | EDSK | 2986 | CYST |
| type269 | | SSSS | | S | | YSSS | | LPKK | | RPS | | DSSG |
| | | Y | | | | SLDA | | YAY | | | | NHRV |
| | | | | | | FDIW | | | | | | F |
| clono | 2342 | GGSF | 2471 | NHSG | 2600 | CARG | 2729 | SGDK | 2858 | QDSK | 2987 | CQAW |
| type270 | | SGY | | S | | TTVV | | LGDK | | RPS | | DSST |
| | | | | | | TPTE | | YAC | | | | VVF |
| | | | | | | YYYM | | | | | | |
| | | | | | | DVW | | | | | | |
| clono | 2343 | GYTF | 2472 | NPKN | 2601 | CARR | 2730 | TGSS | 2859 | GNSN | 2988 | CQSY |
| type272 | | TSY | | GY | | GDFW | | SNIG | | RPS | | DSSL |
| | | | | | | SGYY | | AGYD | | | | SGSV |
| | | | | | | STSQ | | VH | | | | F |
| | | | | | | NIVI | | | | | | |
| | | | | | | HWFD | | | | | | |
| | | | | | | SW | | | | | | |

TABLE 8
Full Heavy Chain (HC) and Light Chain (LC) Sequences for
EPOR/CD131 Binders I

| | SEQ | | SEQ | |
|---|---|---|---|---|
| clono- | ID | | ID | |
| type_id | NO | Full HC AA Sequence | NO | Full LC AA Sequence |
| clonotype | 781 | QVQLVQSGAEVKKPGASVKVSCKA | 917 | SYELTQPPSVSVSPGQTASITC |
| 7 | | SGYTFTSYDINWVRQATGQGLEWM | | SGDKLGDKYACWYQQKPGQSPV |
| | | GWMNPNSGNTGYAQKFQGRVTMTR | | LVIYQDSKRPSGIPERFSGSNS |
| | | NTSISTAYMELSSLRSEDTAVYYC | | GNTATLTISGTQAMDEADYYCQ |
| | | ARWGRGFDPWGQGTLVTVSS | | AWDSSTAVFGGGTKLTVL |
| clonotype | 782 | QVQLVQSGAEVKKPGSSVKVSCKA | 918 | QSALTQPASVSGSPGQSITISC |
| 9 | | SGGTFSSYAISWVRQAPGQGLEWM | | TGTSSDVGGYNYVSWYQQHPGK |
| | | GGIIPIFGTANYAQKFQGRVTITA | | APKLMIYDVSKRPSGVSNRFSG |
| | | DESTSTAYMELSSLRSEDTAVYYC | | SKSGNTASLTISGLQAEDEADY |
| | | ARERLELRWFDPWGQGTLVTVSS | | YCCSYAGSSTWVFGGGTKLTVL |
| clonotype | 783 | QLQLQESGPGLVKPSETLSLTCTV | 919 | QSALTQPASVSGSPGQSITISC |
| 14 | | SGGSISSSSYYWGWIRQPPGKGLE | | TGTSSDVGGYNYVSWYQQHPGK |
| | | WIGSIYYSGSTYYNPSLKSRVTIS | | APKLMIYDVSKRPSGVSNRFSG |
| | | VDTSKNQFSLKLSSVTAADTAVYY | | SKSGNTASLTISGLQAEDEADY |
| | | CARQTDYWFDPWGQGTLVTVSS | | YCCSYAGSSTLVFGGGTKLTVL |
| clonotype | 784 | EVQLVESGGGLVRPGGSLRLSCAA | 920 | QSALTQPASVSGSPGQSITISC |
| 15 | | SGFTFSSYSIHWVRQAPGKGLEWV | | TGTSSDVGGYNYVSWYQQHPGK |
| | | SSISSSSTYIYYADSLKGRFTISR | | APKLMIYEVSNRPSGVSNRFSG |
| | | DNAKNSLYLQMNSLRAEDTAVYYC | | SKSGNTASLTISGLQAEDEADY |
| | | AREGGIAVAGFDYWGQGTLVTVSS | | YCSSYTSSSTLVFGGGTKLTVL |
| clonotype | 785 | EVQLVESGGGLVQPGGSLRLSCAA | 921 | SYELTQPLSVSVALGQTARITC |
| 17 | | SGFTFSSYDMHWVRQATGKGLEWV | | GGNNIGSKNVHWYQQKPGQAPV |
| | | SAIGTAGDTYYPGSVKGRFTISRE | | LVIYRDSNRPSGIPERFSGSNS |
| | | NAKNSLYLQMNSLRAGDTAVYYCA | | GNTATLTISRAQAGDEADYYCQ |
| | | RDSSSWYEDAFDIWGQGTMVTVSS | | VWDSSTVVFGGGTKLTVL |
| clonotype | 786 | EVQLVESGGGLVKPGGSLRLSCAA | 922 | QSALTQPASVSGSPGQSITISC |
| 19 | | SGFTFSDYYMSWIRQAPGKGLEWV | | TGTSSDVGGYNYVSWYQQHPGK |
| | | SYISSSGSTIYYADSVKGRFTISR | | APKLMIYEVSNRPSGVSNRFSG |
| | | DNAKNSLYLQMNSLRAEDTAVYYC | | SKSGNTASLTISGLQAEDEADY |
| | | AREETMVRGVIAYWGQGTLVTVSS | | YCSSYTSSSTVVFGGGTKLTVL |
| clonotype | 787 | EVQLVESGGGLVKPGGSLRLSCAV | 923 | SYELTQPPSVSVSPGQTARITC |
| 22 | | SGFTFSTDSMNWVRQAPGKGLEWV | | SGDALPKKYAYWYQQKSGQAPV |
| | | SSISGSSSYIYYTDSVKGRFTISR | | LVIYEDSKRPSGIPERFSGSSS |
| | | DNAKNSLFLQMNSLRAEDTAVYYC | | GTMATLTISGAQVEDEADYYCY |
| | | ARDWGSFDLWGRGTLVTVSS | | STDSSGNHWVFGGGTKLTVL |
| clonotype | 788 | EVQLVESGGGLIQPGGSLRLSCAA | 924 | QSALTQPPSASGSPGQSVTISC |
| 29 | | SGFTVSSNYMSWVRQAPGKGLEWV | | TGTSSDVGGYNYVSWYQQHPGK |
| | | SVIYSGGSTYYADSVKGRFTISRD | | APKLMIYEVSKRPSGVPDRFSG |
| | | NSKNTLYLQMNSLRAEDTAVYYCA | | SKSGNTASLTVSGLQAEDEADY |
| | | RDGGEYSSSYYFDYWGQGTLVTVS | | YCSSYAGSNNVVFGGGTKLTVL |
| | | S | | |
| clonotype | 789 | QLQLQESGPGLVKPSETLSLTCTV | 925 | LPVLTQPPSASALLGASIKLTC |
| 32 | | SGGSISSSSYYWGWIRQPPGKGLE | | TLSSEHSTYTIEWYQQRPGRSP |
| | | WIGSIYYSGSTYYNPSLKSRVTIS | | QYIMKVKSDGSHSKGDGIPDRF |
| | | VDTSKNQFSLKLSSVTAADTAVYY | | MGSSSGADRYLTFSNLQSDDEA |
| | | CARHDPSFDYWGQGTLVTVSS | | EYHCGESHTIDGQVGVVFGGGT |
| | | | | KLTVL |
| clonotype | 790 | QVQLVQSGAEVKKPGASVKVSCKA | 926 | SYVLTQPPSVSVAPGKTARITC |
| 38 | | SGYTFTSYGISWVRQAPGQGLEWM | | GGNNIGSKSVHWYQQKPGQAPV |
| | | GWISAYNGNTNYAQKLQGRVTMTT | | LVIYYDSDRPSGIPERFSGSNS |
| | | DTSTSTAYMELRSLRSDDTAVYYC | | GNTATLTISRVEAGDEADYYCQ |
| | | ARERSNWDFDYWGQGTLVTVSS | | VWDSSSDHRVFGGGTKLTVL |
| clonotype | 791 | EVQLVESGGGLVQPGGSLRLSCAA | 927 | SYELTQPPSVSVSPGQTASITC |
| 42 | | SGFTFSSYWMSWVRQAPGKGLEWV | | SGDKLGDKYACWYQQKPGQSPV |
| | | ANIKQDGSEKYYVDSVKGRFTISR | | LVIYQDSKRPSGIPERFSGSNS |
| | | DNAKNSLYLQMNSLRAEDTAVYYC | | GNTATLTISGTQAMDEADYYCQ |
| | | ARDGGVRGVITYFDYWGQGTLVTV | | AWDSSNVVFGGGTKLTVL |
| | | SS | | |
| clonotype | 792 | QVQLVQSGAEVKKPGASVKVSCKA | 928 | SYELTQPPSVSVSPGQTASITC |
| 43 | | SGYTFTSYDINWVRQATGQGLEWM | | SGDKLGDKYACWYQQKPGQSPV |
| | | GWMNPNSGNTGYAQKFQGRVTMTR | | LVIYQDSKRPSGIPERFSGSNS |
| | | NTSISTAYMELSSLRSEDTAVYYC | | GNTATLTISGTQAMDEADYYCQ |
| | | ARGRGSSWYWYFDLWGRGTLVTVS | | AWDSSTAVFGGGTKLTVL |
| | | S | | |
| clonotype | 793 | EVQLVESGGGLVQPGGSLRLSCAA | 929 | SYELTQPLSVSVALGQTARITC |
| 44 | | SGFTFSSYDMHWVRQATGKGLEWV | | GGNNIGSKNVHWYQQKPGQAPV |
| | | SAIGTAGDTYYPGSVKGRFTISRE | | LVIYRDSNRPSGIPERFSGSNS |
| | | NAKNSLYLQMNSLRAGDTAVYYCA | | GNTATLTISRAQAGDEADYYCQ |
| | | RGSSSSAFDIWGQGTMVTVSS | | VWDSSTWVFGGGTKLTVL |
| clonotype | 794 | EVQLVESGGGLVKPGGSLRLSCAA | 930 | SYELTQPSSVSVSPGQTARITC |
| 45 | | SGFTFSSYSMNWVRQAPGKGLEWV | | SGDVLAKKYARWFQQKPGQAPV |
| | | SSISSSSSYIYYADSVKGRFTISR | | LVIYKDSERPSGIPERFSGSSS |
| | | DNAKNSLYLQMNSLRAEDTAVYYC | | GTTVTLTISGAQVEDEADYYCY |
| | | ARDGGIAAAGTDYWGQGTLVTVSS | | SAADNNLVFGGGTKLTVL |
| clonotype | 795 | EVQLVESGGGLVKPGGSLRLSCAA | 931 | SYELTQPPSVSVSPGQTARITC |
| 56 | | SGFTFSSYSMNWVRQAPGKGLEWV | | SGDALPKKYAYWYQQKSGQAPV |
| | | SSISSSSSYIYYADSVKGRFTISR | | LVIYEDSKRPSGIPERFSGSSS |
| | | DNAKNSLYLQMNSLRAEDTAVYYC | | GTMATLTISGAQVEDEADYYCY |
| | | ARADSLTGGFFDYWGQGTLVTVSS | | STDSSGNHSWVFGGGTKLTVL |
| clonotype | 796 | QVQLVQSESELKKPGASVKVSCKA | 932 | QAVVTQEPSLTVSPGGTVTLTC |
| 57 | | SGYTFISYAMSWVRQAPGQGLEWM | | GSSTGAVTSGHYPYWFQQKPGQ |
| | | GWINTNTGNPTYAQGFTGREVESL | | APRTLIYDTSNKHSWTPARFSG |
| | | DTSVSTAYLQISSLKAEDTAVYYC | | SLLGGKAALTLSGAQPEDEAEY |
| | | AERGWNYDYWGQGTLVTVSS | | YCLLSYSGAWVFGGGTKLTVL |
| clonotype | 797 | EVQLVESGGGLIQPGGSLRLSCAA | 933 | QSALTQPASVSGSPGQSITISC |
| 65 | | SGFTVSSNYMSWVRQAPGKGLEWV | | TGTSSDVGGYNYVSWYQQHPGK |
| | | SVIYSGGSTYYADSVKGRFTISRD | | APKLMIYEVSNRPSGVSNRFSG |
| | | NSKNTLYLQMNSLRAEDTAVYYCA | | SKSGNTASLTISGLQAEDEADY |
| | | RDSTTVTLFDYWGQGTLVTVSS | | YCSSYTSSSTYVFGTGTKVTVL |
| clonotype | 798 | EVQLVESGGGLVKPGGSLRLSCAA | 934 | QSALTQPPSASGSPGQSVTISC |
| 68 | | SGFTFSSYSMNWVRQAPGKGLEWV | | TGTSSDVGGYNYVSWYQQHPGK |
| | | SSISSSSSYIYYADSVKGRFTISR | | APKLMIYEVSKRPSGVPDRFSG |
| | | DNAKNSLYLQMNSLRAEDTAVYYC | | SKSGNTASLTVSGLQAEDEADY |
| | | ARGIAVAGPHAFDIWGQGTMVTVS | | YCSSYAGSNNFVVFGGGTKLTV |
| | | S | | L |
| clonotype | 799 | EVQLVESGGGLIQPGGSLRLSCAA | 935 | QSVLTQPPSVSGAPGQRVTISC |
| 70 | | SGFTVSSNYMSWVRQAPGKGLEWV | | TGSSSNIGAGYDVHWYQQLPGT |
| | | SVIYSGGSTYYADSVKGRFTISRD | | APKLLIYGNSNRPSGVPDRFSG |
| | | NSKNTLYLQMNSLRAEDTAVYYCA | | SKSGTSASLAITGLQAEDEADY |
| | | RGYSGSYAYWGQGTLVTVSS | | YCQSYDSSLSGYVFGTGTKVTV |
| | | | | L |
| clonotype | 800 | QVQLQQSGPGLVKPSQTLSLTCAI | 936 | QSALTQPASVSGSPGQSITISC |
| 72 | | SGDSVSSNSAAWNWIRQSPSRGLE | | TGTSSDVGGYNYVSWYQQHPGK |
| | | WLGRTYYRSKWYNDYAGSVKSRII | | APKLMIYDVSKRPSGVSNRFSG |
| | | INPDTSKNQLSLQLKSVTPEDTAV | | SKSGNTASLTISGLQAEDEADY |
| | | YYCARKWELRDAFDIWGQGTMVTV | | YCCSYAGRSTLGIDWVFGGGTK |
| | | SS | | LTVL |
| clonotype | 801 | EVQLVQSGAVVKKPGESLKISCKG | 937 | SYVLTQPPSVSVAPGKTARITC |
| 74 | | SGYSFSSYWIGWVRQMPGKGLEWM | | EGDNIGSESVHWYQQKPGQAPV |
| | | GIIYPGDSDTRYSPSFQGQVTISA | | LVIYFDSDRPSGIPERFSGSNS |
| | | DKSISTAYLQWSSLQASDTAMYFC | | GITATLTISRVEAGDEADFYCQ |
| | | AKRRMTGSHSWFDPWGQGTLVTVS | | VWDNGSDHVVFGGGTKLTVL |
| | | S | | |
| clonotype | 802 | EVQLVESGGGLVQPGGSLRLSCAA | 938 | QSALTQPASVSGSPGQSITISC |
| 215 | | SGFTFSSYWMHWVRQAPGKGLVWV | | TGTSSDVGGYNYVSWYQQHPGK |
| | | SRINSDGSSTSYADSVKGRFTISR | | APKLMIYEVSNRPSGVSNRFSG |
| | | DNAKNTLYLQMNSLRAEDTAVYYC | | SKSGNTASLTISGLQAEDEADY |
| | | ARIDYWGQGTLVTVSS | | YCSSYTSSSTWVFGGGTKLTVL |
| clonotype | 803 | EVQLVESGGGLVQPGGSLRLSCAA | 939 | SYELTQPPSVSVSPGQTARITC |
| 216 | | SGFTFSSYWMSWVRQAPGKGLEWV | | SGDALPKKYAYWYQQKSGQAPV |
| | | ANIKQDGSEKYYVDSVKGRFTISR | | LVIYEDSKRPSGIPERFSGSSS |
| | | DNAKNSLYLQMNSLRAEDTAVYYC | | GTMATLTISGAQVEDEADYYCY |
| | | ARDGGTPGQGTLVTVSS | | STDSSGNHRVFGGGTKLTVL |
| clonotype | 804 | QVQLQESGPGLVKPSETLSLTCTV | 940 | SYELTQPSSVSVSPGQTARITC |
| 219 | | SGGSISSYYWSWIRQPPGKGLEWI | | SGDVLAKKYARWFQQKPGQAPV |
| | | GYIYYSGSTNYNPSLKSRVTISVD | | LVIYKDSERPSGIPERFSGSSS |
| | | TSKNQFSLKLSSVTAADTAVYYCA | | GTTVTLTISGAQVEDEADYYCY |
| | | IVGARFDYWGQGTLVTVSS | | SAADNNLVFGGGTKLTVL |
| clonotype | 805 | EVQLVESGGGLVKPGGSLRLSCAA | 941 | SYELTQPPSVSVSPGQTARITC |
| 221 | | SGFTFINAWMSWVRQAPGKGLEWV | | SADALPNQYAYWYQQKPGQAPV |
| | | GRIKSKTDGGTTDYAAPVKGRFTI | | LVIYKDSERPSGIPERFSGSSS |
| | | SRDDSKNTLYLQMNSLKTEDTAVY | | GTTVTLTISGVQAEDEADYYCQ |
| | | YCTTGGTHWGQGTLVTVSS | | SADSSGTWVFGGGTKLTVL |
| clonotype | 806 | EVQLVESGGGLVKPGGSLRLSCAA | 942 | SYELTQPPSVSVSPGQTARITC |
| 223 | | SGFTFSNAWMSWVRQAPGKGLEWV | | SADALSKQYAYWYQQKPGQAPV |
| | | GRIKSKTDGGTTDYAAPVKGRFTI | | LVIYKDSERPSGIPERFSGSSS |
| | | SRDDSKNTLYLQMNSLKTEDTAVY | | GTTVTLTISGVQAEDEADYYCQ |
| | | YCTTGGYRWGQGTLVTVSS | | SADSSGTNWVFGGGTKLTVL |
| clonotype | 807 | EVQLVESGGGLVKPGGSLRLSCAA | 943 | SYELTQPPSVSVSPGQTARITC |
| 224 | | SGFTFSNAWMSWVRQAPGKGLEWV | | SGDALPKKYAYWYQQKSGQAPV |
| | | GRIKSKTDGGTTDYAAPVKGRFTI | | LVIYEDSKRPSGIPERFSGSSS |
| | | SRDDSKNTLYLQMNSLKTEDTAVY | | GTMATLTISGAQVEDEADYYCY |
| | | YCTTDLYYWGQGTLVTVSS | | STDSSGNHRVFGGGTKLTVL |
| clonotype | 808 | QVQLVQSGAEVKKPGASVKVSCKA | 944 | QSALTQPASVSGSPGQSITISC |
| 225 | | SGYTFTSYGISWVRQAPGQGLEWM | | TGTSSDVGGYNYVSWYQQHPGK |
| | | GWISAYNGNTNYAQKLQGRVTMTT | | APKLMIYEVSNRPSGVSNRFSG |
| | | DTSTSTAYMELRSLRSDDTAVYYC | | SKSGNTASLTISGLQAEDEADY |
| | | ARGWYFDYWGQGTLVTVSS | | YCSSYTSSSTLVFGGGTKLTVL |
| clonotype | 809 | EVQLVESGGGLVQPGGSLRLSCAA | 945 | QSALTQPASVSGSPGQSITISC |
| 226 | | SGFTFSTYAMNWVRQAPGKGLEWV | | TGTSSDVGGYNYVSWYQQHPGK |
| | | SAISGGGGSTYYADSVKGRFTISR | | APKLMIYEVSNRPSGVSNRFSG |
| | | DNSKNTLYLQMNSLRAEDTAVYYC | | SKSGNTASLTISGLQAEDEADY |
| | | AKRVFFDYWGQGTLVTVSS | | SCSSYTISSTWVFGGGTKLTVL |
| clonotype | 810 | EVQLVESGGGLVQPGGSLRLSCAA | 946 | SYELTQPPSVSVSPGQTARITC |
| 228 | | SGFTFSSYDMHWVRQATGKGLEWV | | SGDALPKKYAYWYQQKSGQAPV |
| | | SAIGTAGDTYYPGSVKGRFTISRE | | LVIYEDSKRPSGIPERFSGSSS |
| | | NAKNSLYLQMNSLRAGDTAVYYCA | | GTMATLTISGAQVEDEADYYCY |
| | | RDLGRVFDYWGQGTLVTVSS | | STDSSGNHRVFGGGTKLTVL |
| clonotype | 811 | VVQLVESGGGLVQPGGSLRLSCAA | 947 | QSALTQPASVSGSPGQSITISC |
| 229 | | SGFTFSRYWMSWVRQAPGKGLEWV | | TGTSSDVGGYDYVSWYQQHPGK |
| | | ANINQDGSEEYYVDSVKGRFTISR | | APKFMISGVSNRPSGVSNRFSG |
| | | DNAKSSLSLQMNSLRAEDTALYYC | | SKSGNTASLTISGLQAEDEADY |
| | | ATDLNWNGYWGQGTLVTVSS | | YCSSYTRSRTWVFGGGTKLTVL |
| clonotype | 812 | EVQLVESGGGLVQPGGSLRLSCAA | 948 | QAVLTQPASLSASPGASASLTC |
| 230 | | SGFTFSRCDMYWVRQATGKGLEWV | | TLRSGINVGTYRIYWYQQKPGS |
| | | SAIGAAGDTYYPGSVKGRFTISRE | | PPQYLLRYKSDSDKQQGSGVPS |
| | | NAKNSLYLQMNSLRAGDTAVYYCA | | RFSGSKDASANAGILLISGLQS |
| | | TGYNWNPDYWGQGTLVTVSS | | EDEADYYCMIWHSSASVFGGGT |
| | | | | KLTVL |
| clonotype | 813 | QVQLVESGGGVVQPGRSLRLSCEA | 949 | SYELTQPSSVSVSPGQTARITC |
| 231 | | SGFTFINYGMHWVRQAPGKGLEWV | | SGDVLAKKYARWFQQKPGQAPV |
| | | AVIWYDGSNKYYADSVKGRFTISR | | LVIYKDSERPSGIPERFSGSSS |
| | | DNSKNTLYLQMNSLRAEDTAVYYC | | GTTVTLTISGAQVEDEADYYCY |
| | | ARDRSSSSDYWGQGTLVIVSS | | SAADNNRVFGGGTKLTVL |
| clonotype | 814 | EVQLVESGGGLVQPGGSLRLSCAA | 950 | SYELTQPSSVSVSPGQTARITC |
| 232 | | SGFTFSSYWMHWVRQAPGKGLVWV | | SGDVLAKKYARWFQQKPGQAPV |
| | | SRINSDGSSTSYADSVKGRFTISR | | LVIYKDSERPSGIPERFSGSSS |
| | | DNAKNTLYLQMNSLRAEDTAVYYC | | GTTVTLTISGAQVEDEADYYCY |
| | | AGITGTYFDYWGQGTLVTVSS | | SAADNNLVFGGGTKLTVL |
| clonotype | 815 | EVQLVESGGGLVQPGRSLRLSCAA | 951 | SYELTQPPSVSVSPGQTARITC |
| 233 | | SGFTEDDYAMHWVRQAPGKGLEWV | | SGDALPKKYAYWYQQKSGQAPV |
| | | SGISWNSGSIGYADSVKGRFTISR | | LVIYEDSKRPSGIPERFSGSSS |
| | | DNAKNSLYLQMNSLRAEDTALYYC | | GTMATLTISGAQVEDEADYYCY |
| | | AKDSGYSPDYWGQGTLVTVSS | | STDSSGKGVFGGGTKLTVL |
| clonotype | 816 | QVQLVQSGAEVKKPGASVKVSCKA | 952 | SYELTQPPSVSVSPGQTARITC |
| 234 | | SGYTFTSYGISWVRQAPGQGLEWM | | SGDALPKKYAYWYQQKSGQAPV |
| | | GWISAYNGNTNYAQKLQGRVTMTT | | LVIYEDSKRPSGIPERFSGSSS |
| | | DTSTSTAYMELRSLRSDDTAVYYC | | GTMATLTISGAQVEDEADYYCY |
| | | ARDRPYYFDYWGQGTLVTVSS | | STDSSGNHRVFGGGTKLTVL |
| clonotype | 817 | QVQVVQSGAEVRKSGASVKVSCKA | 953 | SSELTQDPAVSVALGQTVRITC |
| 235 | | SGYTFTSYYIHWVRQVPGQGLEWM | | QGDSLRSYYASWYQQKPGQAPV |
| | | GLINPSGGSTIYAQKFQGRVTMTR | | LVIYGKNNRPSGIPDRESGSSS |
| | | DTSTSSVYMELSSLRSEDTAAYYC | | GNTASLTITGAQAEDEADYYCN |
| | | ARGGWGTMDVWGKGTTVTVSS | | SRDSSGNHYVFGTGTKVTVL |
| clonotype | 818 | EVQLVESGGGLVQPGGSLRLSCAA | 954 | SSELTQDPAVSVALGQTVRITC |
| 236 | | SGFTFSSYDMHWVRQATGKGLEWV | | QGDSLRSYYASWYQQKPGQAPV |
| | | SAIGTAGDTYYPGSVKGRFTISRE | | LVIYGKNNRPSGIPDRFSGSSS |
| | | NAKNSLYLQMNSLRAGDTAVYYCA | | GNTASLTITGAQAEDEADYYCN |
| | | RAWELDAFDIWGQGTMVTVSS | | SRDSSGNHVVFGGGTKLTVL |
| clonotype | 819 | EVQLVESGGGLVQPGGSLRLSCAA | 955 | SYELTQPPSVSVSPGQTARITC |
| 237 | | SGFTFSSYAMSWVRQAPGKGLEWV | | SGDALPKKYAYWYQQKSGQAPV |
| | | SAISGSGGSTYYADSVKGRFTISR | | LVIYEDSKRPSGIPERFSGSSS |
| | | DNSKNTLYLQMNSLRAEDTAVYYC | | GTMATLTISGAQVEDEADYYCY |
| | | AKDNWNYFDYWGQGTLVTVSS | | STDSSGNHRVFGGGTKLTVL |
| clonotype | 820 | QVQLVESGGGVVQPGRSLRLSCAA | 956 | SYELTQPPSVSVSPGQTARITC |
| 238 | | SGFTFSSYGMHWVRQAPGKGLEWV | | SGDALPKKYAYWYQQKSGQAPV |
| | | AVIWYDGSNKYYADSVKGRFTISR | | LVIYEDSKRPSGIPERFSGSSS |
| | | DNSKNTLYLQMNSLRAEDTAVYYC | | GTMATLTISGAQVEDEADYYCY |
| | | ARVYNWIFDYWGQGTLVTVSS | | STDSSGNHRVFGGGTKLTVL |
| clonotype | 821 | EVQVMESGGGLVKPGGSLRLSCAA | 957 | QSALTQPASVSGSPGQSITISC |
| 239 | | SGFSFSSHSLNWVRQAPGKGLEWV | | TGTNNDVGYYNYVSWYQQHPDK |
| | | SSISGISNYIAYADSVRGRFTISR | | APKLMIYDVIKRPSGVSDRFSG |
| | | DNAKNSLFLQMNSLRAEDTGVYYC | | SKSGNTASLTISGLQAEDEADY |
| | | ARITVVSFDYWGQGTLVTVTS | | YCCSYAGSSTWVFGGGTKLSVL |
| clonotype | 822 | EVQLVESGGGLVQPGGSLRLSCAA | 958 | QSALTQPPSASGSPGQSVTISC |
| 240 | | SGFTFSSYAMSWVRQAPGKGLEWV | | TGTSSDVGGYNYVSWYQQHPGK |
| | | SAISGSGGNTYNADSVKGRFTISR | | APKLMIYEVSKRPSGVPDRFSG |
| | | DNSKNTLYLQMNSLRAEDTAVYYC | | SKSGNTASLTVSGLQAEDEADY |
| | | AKAAAGKGDYWGQGTLVTVSS | | YCSSYSGSNNYVFGTGTKVTVL |
| clonotype | 823 | QVQLVQSGAEVKKPGASVKVSCKA | 959 | QSALTQPASVSGSPGQSITISC |
| 241 | | SGYTFTSYYIHWVRQAPGQGLEWM | | TGTSSDVGNYNYVSWYQQHPGK |
| | | GIINPSGGTTNYAQKFQGRVTMTR | | VPKLMIYEVIYRPSGVSNRFSG |
| | | DTSTSTVYMELSSLRSEDTAVYYC | | SKSGNTASLTISGLQAEDEADY |
| | | ARGDWGTMDVWGKGTTVTVSS | | YCTSYTRNNTYVFGSGTKVTVL |
| clonotype | 824 | EVQLVESGGGLVQPGGSLRLSCAA | 960 | QSVLTQPPSVSGAPGQRVTISC |
| 242 | | SGFTFSSYWMSWVRQAPGKGLEWV | | TGSSSNIGAGYDVHWYQQLPGT |
| | | ANIKQDGSEKYYVDSVKGRFTISR | | APKLLIYGNSNRPSGVPDRESG |
| | | DNAKNSLYLQMNSLRAEDTAVYYC | | SKSGTSASLAITGLQAEDEADY |
| | | ARDGTGWFDPWGQGTLVTVSS | | YCQSYDSSLSGWVFGGGTKLTV |
| | | | | L |
| clonotype | 825 | QVQLVQSGAEVKKPGASVKVSCKA | 961 | QSALTQPASVSGSPGQSITISC |
| 243 | | SGYTFTSYGISWVRQAPGQGLEWM | | TGTSSDVGGYNYVSWYQQHPGK |
| | | GWISAYNGNTNYAQKLQGRVTMTT | | APKLMIYDVSKRPSGVSNRFSG |
| | | DTSTSTAYMELRSLRSDDTAVYYC | | SKSGNTASLTISGLQAEDEADY |
| | | ARRGTVVFDYWGQGTLVTVSS | | YCCSYAGSSTYVVFGGGTKLTV |
| | | | | L |
| clonotype | 826 | QVQLVQSGAEVKKPGASVKVSCKA | 962 | QSALTQPASVSGSPGQSITISC |
| 244 | | SGYTFTNYGISWVRQAPGQGLEWM | | TGTSSDVGGYNYVSWYQQHPGK |
| | | GWISAYNGNTNYAQNLQGRVTMTT | | APKLMIYDVSKRPSGVSNRFSG |
| | | DTSTSTAYMELRSLRSDDTAVYYC | | SKSGNTASLTISGLQAEDEADY |
| | | ARPLSGTLDNWGQGTLVTVSS | | YCCSYAGRSTLGIDWVFGGGTK |
| | | | | LTVR |
| clonotype | 827 | EVQLVESGGGLVQPGGSLRLSCAA | 963 | SSELTQDPAVSVALGQTVRITC |
| 246 | | SGFTFSSYSMNWVRQAPGKGLEWV | | QGDSLRSYYASWYQQKPGQAPV |
| | | SYISSSSSTIYYADSVKGRFTISR | | LVIYGKNNRPSGIPDRFSGSSS |
| | | DNAKNSLYLQMNSPRDEDTAVYYC | | GNTASLTITGAQAEDEADYYCN |
| | | ARESIAALFDYWGQGTLVTVSS | | SRDSSGNHLVFGGGTKLTVL |
| clonotype | 828 | EVQLVESGGGLVQPGGSLRLSCAA | 964 | SYELTQPPSVSVSLGQMARITC |
| 247 | | SGFTFSSYDMHWVRQATGKGLEWV | | SGEALPKKYAYWYQQKPGQFPV |
| | | SAIGTAGDTYYPGSVKGRFTISRE | | LVIYKDSERPSGIPERFSGSSS |
| | | NAKNSLYLQMNSLRAGDTAVYYCA | | GTIVTLTISGVQAEDEADYYCL |
| | | RGDHSYGGLDYWGQGTLVTVSS | | SADSSGTYRVFGGGTKLTVL |
| clonotype | 829 | QVQLVQSGAEVEKPGASVKVSCKA | 965 | QSALTQPASVSGSPGQSITISC |
| 249 | | SGYTFTSYDIYWVRQATGQGLEWM | | TGTSSDVGGYNYVSWYQQHPGK |
| | | GWMIPNSGNTGYAQRFEDRVTMTR | | APKLMIYEVSHRPSGVSNRFSG |
| | | STSMNTAYMELNSLRSEDTAVYYC | | SKSGNTASLTISGLQAEDESDY |
| | | ARGDWAWSFDLWGRGTLVTVSS | | YCSSYTSSSSLVFGGGTKLTVL |
| clonotype | 830 | QVQLVQSGAEVKKPGASVKVSCKA | 966 | QSALTQPASVSGSPGQSITISC |
| 250 | | SGYTFTSYYMHWVRQAPGQGLEWM | | TGTSSDVGGYNYVSWYQQHPGK |
| | | GIINPSGGSTSYAQKFQGRVTMTR | | APKLMIYEVSNRPSGVSNRFSG |
| | | DTSTSTVYMELSSLRSEDTAVYYC | | SKSGNTASLTISGLQAEDEADY |
| | | ARGLRRDWFDPWGQGTLVTVSS | | YCSSYTSSSTWVFGGGTKLTVL |
| clonotype | 831 | EVQLVESGGGLVKPGGSLRLSCAA | 967 | QSALTQPRSVSGSPGQSVTISC |
| 251 | | SGFTFSNAWMSWVRQAPGKGLEWV | | TGTSSDVGGYNYVSWYQQHPGK |
| | | GRIKSKTDGGTTDYAAPVKGRFTI | | APKLMIYDVTTRPSGVPDRFSG |
| | | SRDDSKNTLYLQMNSLKTEDTAVY | | SKSGNTASLTISGLQAEDEADY |
| | | YCTTGTGRSDYWGQGTLVTVSS | | YCCSYSGSYTYVFGTGTKVTVL |
| clonotype | 832 | QVQLVESGGGVVQPGRSLRLSCAA | 968 | QSALTQPASVSGSPGQSITISC |
| 252 | | SGFTFSNYGMHWVRQAPGKGLDWV | | TGTSSDVGGYNYVSWYQQHPGK |
| | | AVIWYDGNNEYYADSVKDRFTISR | | VPKLMIYEVSNRPSGVSNRFSG |
| | | DNSQNTLYLQMNSLRAEDRAVYYC | | SKSGNTASLTISGLQAEDEADY |
| | | VRGGVGDGFDMWGQGTMVTVSS | | YCISYTNTNTRVFGGGTKLTVL |
| clonotype | 833 | QVQLVESGGGVVQPGRSLRLSCAA | 969 | QSALTQPASVSGSPGQSITISC |
| 253 | | SGFTFSSYGMHWVRQAPGKGLEWV | | TGTSSDVGGYNYVSWYQQHPGK |
| | | AVIWYDGSNKYYADSVKGRFTISR | | APKLMIYEVSNRPSGVSNRFSG |
| | | DNSKNTLYLQMNSLRAEDTAVYYC | | SKSGNTASLTISGLQAEDEADY |
| | | ASVGSYGYFQHWGQGTLVTVSS | | YCSSYTSSSTWVFGGGTKLTVL |
| clonotype | 834 | EVQLVESGGGVVRPGGSLRLSCAA | 970 | QSALTQPPSASGSPGQSVTISC |
| 254 | | SGFTEDDYGMSWVRQAPGKGLEWV | | TGTSSDVGGYNYVSWYQQHPGK |
| | | SGINWNGGSTGYADSVKGRFTISR | | APKLMIYEVSKRPSGVPDRESG |
| | | DNAKNSLYLQMNSLRAEDTALYYC | | SKSGNTASLTVSGLQAEDEADY |
| | | ARKGNWNSFDYWGQGTLVTVSS | | YCSSYAGSNNWVFGGGTKLTVL |
| clonotype | 835 | EVQLVESGGGLVKPGGSLRLSCAA | 971 | QSALTQPPSASGSPGQSVTISC |
| 255 | | SGFTFSSYSMNWVRQAPGKGLEWV | | TGTSSDVGGYNYVSWYQQHPGK |
| | | SSISSSSSYIYYADSVKGRFTISR | | APKLMIYEVSKRPSGVPDRFSG |
| | | DNAKNSLYLQMNSLRAEDTAVYYC | | SKSGNTASLTVSGLQAEDEADY |
| | | ARDSAYYTFDYWGQGTLVTVSS | | YCSSYAGSNNFWVFGGGTKLTV |
| | | | | L |
| clonotype | 836 | EVQLVESGGGLVQPGGSLRLSCAA | 972 | QSALTQPASVSGSPGQSITISC |
| 256 | | SGFTFSSYAMSWVRQAPGKGLEWV | | TGTSSDVGGYNYVSWYQQHPGK |
| | | SAISGSGGSTYYADSVKGRFTISR | | APKLMIYEVSNRPSGVSNRFSG |
| | | DNSKNTLYLQMNSLRAEDTAVYYC | | SKSGNTASLTISGLQAEDEADY |
| | | GSGWYEGAFDYWGQGTLVTVSS | | YCSSYTSSSTYWVFGGGTKLTV |
| | | | | L |
| clonotype | 837 | EVQLVESGGGLVQPGGSLRLSCAA | 973 | SYELTQPPSVSVSPGQTASITC |
| 259 | | SGFTFSSYDMHWVRQATGKGLEWV | | SGDKLGDKYACWYQQKPGQSPV |
| | | SAIGTAGDTYYPGSVKGRFTISRE | | LVIYQDSKRPSGIPERFSGSNS |
| | | NAKNSLYLQMNSLRAGDTAVYYCA | | GNTATLTISGTQAMDEADYYCQ |
| | | RDRDSSHDAFDIWGQGTMVTVSS | | AWDSSTVVFGGGTKLTVL |
| clonotype | 838 | EVQLVESGGGVVRPGGSLRLSCAA | 974 | SYELTQPSSVSVSPGQTARITC |
| 260 | | SGFPFDDFGLNWVRQAPGKGLEWV | | SGDVLAKKYARWFQQKPGQAPV |
| | | SGINWNGGTTTYADSVKGRFTISR | | LVIYKDSERPSGIPERFSGSSS |
| | | DNAKKSLYLQMSSLRVEDTALFYC | | GTTVTLTISGAQVEDEADYYCY |
| | | VRDEIWNYYFDYWGQGTLVTVSS | | SAADNNRVFGGGTKLTVL |
| clonotype | 839 | EVQMVESGGGRVKPGGSLRLSCTA | 975 | SYELTQPPSVSVSPGQTARITC |
| 262 | | SGFSISINNMNWVRQAPGKGLEWV | | SGDALPKKYAYWYQQKSGQAPV |
| | | SSISSSSTYIYYADSVKGRFTISR | | LVIYEDSKRPSGIPERFSGSSS |
| | | DNAENSLYLQMNSLRAEDTGVYYC | | GTMATLTISGAQVEDEADYYCY |
| | | ARDSYDFHAFDIWGQGTMVTVSS | | STDSSGNHRVFGGGTKLTVL |
| clonotype | 840 | EVQLVESGGGLVQPGGSLRLSCAA | 976 | SSELTQDPAVSVALGQTVRITC |
| 264 | | SGFTFSSYWMHWVRQAPGKGLVWV | | QGDSLRSYYASWYQQKPGQAPV |
| | | SRINSDGSSTSYADSVKGRFTISR | | LVIYGKNNRPSGIPDRFSGSSS |
| | | DNAKNTLYLQMNSLRAEDTAVYYC | | GNTASLTITGAQAEDEADYYCN |
| | | ARVGWGGHAFDIWGQGTMVTVSS | | SRDSSGNHVVFGGGTKLTVL |
| clonotype | 841 | EVQLVESGGGLVKPGGSLRLSCAA | 977 | QSALTQPASVSGSPGQSITISC |
| 265 | | SGFTFSTYSMNWVRQAPGKGLEWV | | TGTSSDVGGYNYVSWYQQHPGK |
| | | SSISSSSTYIYYADSVKGRFTISR | | APKLMIYEVSNRPSGVSNRFSG |
| | | DNAKNSLYLQMNSLRAEDTAVYYC | | SKSGNTASLTISGLQAEDEADY |
| | | ARGYNWNYVGDYWGQGTLVTVSS | | YCSSYTSSSTLVFGGGTKLTVL |
| clonotype | 842 | EVQLVESGGGLVQPGGSLRLSCAA | 978 | QSVLTQPPSVSGAPGQRVTISC |
| 266 | | SGFTFSSYAMSWVRQAPGKGLEWV | | TGSSSNIGAGYDVHWYQQLPGT |
| | | SAISGSGGSTYYADSVKGRFTISR | | APKLLIYGNSNRPSGVPDRFSG |
| | | DNSKNTLYLQMNSLRAEDTAVYYC | | SKSGTSASLAITGLQAEDEADY |
| | | AKDANWGYAFDIWGQGTMVTVSS | | YCQSYDSSLSGSVFGGGTKLTV |
| | | | | L |
| clonotype | 843 | QVQLVQSVSEVNKPGASVKVSCKA | 979 | SYELTQPPSVSVSPGQTASITC |
| 270 | | SGYTFTTYGISWVRQAPGQGLEWM | | SRDKLGDKYACWYQQKPGQSPV |
| | | GWISGYSGYTSYAQKFQGRLTMTT | | LVIYQDSKRPSGIPERFSGSNS |
| | | DTSANTAYMELRSLRSDDTAVYYC | | GNTATLTISGTQAMDEADYYCQ |
| | | ARRFIWNYGDFDYWGQGTLVTVSS | | AWDSSTVVFGGGTKLTVL |
| clonotype | 844 | EVQLVESGGDLVQPGGSLRLSCAA | 980 | SYELTQPPSVSVSPGQTASITC |
| 271 | | SGFTFSSYSMNWVRQAPGKGLEWV | | SGDKLGDRYACWYQQKPGQSPV |
| | | SYISRSSGTIYYADSVKGRFTISR | | LVIYQGSKRPSGIPERFSGSNS |
| | | DNAKNSLYLQMNSLRDEDTAVYYC | | GNTATLTISGTQAMDEADYYCQ |
| | | ARGRLGIEDYFDYWGQGTLVTVSS | | AWDSSTVVFGGGTKLTVL |
| clonotype | 845 | EVQLVESGGGLVQPGGSLRLSCAA | 981 | SSELTQDPAVSVALGQTVRITC |
| 272 | | SGFTFNRYNMNWVRQAPGKGLEWV | | QGDSLRSYYASWYQQKPGQAPV |
| | | SYISSSSDTIYYADSVKGRFTISR | | LVIYGKNNRPSGILDRFSGSSS |
| | | DNAKNSLYLQMNSLRDEDTAMYYC | | GNTASLTITGAQAEDEGDYYCN |
| | | ARECYSSSWAFDYWGQGTLVTVSS | | SRDSSGWVFGGGTKLTVL |
| clonotype | 846 | QVQLQESGPGLVKPSGTLSLTCAI | 982 | SYELTQPPSVSVSPGQTANITC |
| 273 | | SGGSISSSNWWSWVRQPPGKGLEW | | SGDKLENKYTCWYQQKPGQSPL |
| | | IGEIYHSGSTNFNPSLKSRVTISV | | VVIYQDNKRPSGIPERFSGSNS |
| | | DKSKNQFSLHLSSVTAADTAVYYC | | GNTATLTISGTQAMDEADYYCQ |
| | | ARDVGVDGRGFDYWGQGIVVIVSS | | AWDSTTAWVFGGGTKLTVL |
| clonotype | 847 | EVQLVESGGGLVQPGGSLRLSCGV | 983 | SSELTQDPAVSVALGQTVRITC |
| 274 | | SGFTFSIYWMSWVRQAPGKGLEWV | | QGDNIRNYYASWYQQKPGQAPL |
| | | ANINLDGSEKYHVDSVKGRFTISR | | LVISGKNNRPSGIPDRFSGSSS |
| | | DNAKNSLFLQMTSLRAEDTAVYYC | | GNTASLTITGAQAEDEAYYYCY |
| | | ARDILWSGGYLDVWGKGTTVTVSS | | SRDSSGSLWIFGGGTKLTVL |
| clonotype | 848 | EVQLVESGGGLVQPGGSLRLSCAA | 984 | SSELTQDPAVSVALGQTVRITC |
| 275 | | SGFTFSGYWMTWVRQAPGKGLEWV | | QGDSLRRYYASWYQQKPGQAPV |
| | | ANIKHDGSEKYYVDSVKGRFTISR | | LVIYGKDNRPSGIPDRFSGSSS |
| | | DNAQNSLYLQMNSLRAEDTAVYYC | | GNTASLTITGTQAEDEADYYCN |
| | | ARKQLWLNWYFDFWGRGILVTVSS | | SRDSSGNHLVFGGGTKLTVL |
| clonotype | 849 | QVQLVQSGAEVKKPGASVKVSCKA | 985 | SSELTQDPAVSVALGQTVRITC |
| 276 | | SGYTFTTYGISWVRQAPGQGLEWM | | QGDSLRSYYASWYQQKPGQAPV |
| | | GWISAFNGNTNYAQNLQGRVTMTT | | LVIYGKNNRPSGIPDRFSGSSS |
| | | DTSTSTAYMELRSLRSDDTAVYYC | | GNTASLTITGAQAEDEADYYCN |
| | | ARENNWNYGWFDPWGQGTLVTVSS | | SRDSSGNHYVFGTGTKVTVL |
| clonotype | 850 | EVQLVESGGGLVQPGGSLRLSCAA | 986 | SYELTQPPSVSVSPGQTARITC |
| 277 | | SGFTFSSYDMHWVRQATGKGLEWV | | SGDALPKKYAYWYQQKSGQAPV |
| | | SAIGTAGDTYYPGSVKGRFTISRE | | LVIYEDSKRPSGIPERFSGSSS |
| | | NAKNSLYLQMNSLRAGDTAVYYCA | | GTMATLTISGAQVEDEADYYCY |
| | | REGYGDYPLPMDVWGKGTTVTVSS | | STDSSGNHRVFGGGTKLTVL |
| clonotype | 851 | EVQLVESGGGLVKPGGSLRLSCAA | 987 | SYELTQPPSVSVSPGQTARITC |
| 278 | | SGFTFSNAWMSWVRQAPGKGLEWV | | SGDALPKKYAYWYQQKSGQAPV |
| | | GRIKSKTDGGTTDYAAPVKGRFTI | | LVIYEDSKRPSGIPERFSGSSS |
| | | SRDDSKNTLYLQMNSLKTEDTAVY | | GTMATLTISGAQVEDEADYYCY |
| | | YCTTDNWNSYFDYWGQGTLVTVSS | | STDSSGNHRVFGGGTKLTVL |
| clonotype | 852 | EVQLVESGGGLVQPGGSLRLSCAA | 988 | SYELTQPPSVSVSPGQTARITC |
| 279 | | SGFTFSSYAMSWVRQAPGKGLEWV | | SGDALPKKYAYWYQQKSGQAPV |
| | | SAISGGGGSTYYADSVKGRFTISR | | LVIYEDSKRPSGIPERFSGSSS |
| | | DNSKNTLYLQMNSLRAEDTAVYYC | | GTMATLTISGAQVEDEADYYCY |
| | | AGNSGYDSPYFDYWGQGTLVTVSS | | STDSSGNHRVFGGGTKLTVL |
| clonotype | 853 | QVQLVESGGGVVQPGRSLRLSCAA | 989 | SYELTQPPSVSVSPGQTARITC |
| 280 | | SGFTFSSYGMHWVRQAPGKGLEWV | | SGDALPKKYAYWYQQKSGQAPV |
| | | AVIWYDGSNKYYADSVKGRFTISR | | LVIYEDIKRPSGIPERFSGSSS |
| | | DNSKNTLYLQMNSLRAEDTAVYYC | | GTMATLTISGAQVEDEADYYCY |
| | | AREYSSSSDWFDPWGQGTLVTVSS | | STDSSGNHRVFGGGTKLTVL |
| clonotype | 854 | EVQLVESGGGLVKPGGSLRLSCAA | 990 | QSALTQPRSVSGSPGQSVTISC |
| 281 | | SGFTFSSYSMNWVRQAPGKGLEWV | | TGTSSDVGGYNYVSWYQQHPGK |
| | | SSISSSSSYIYYADSVKGRFTISR | | APKLMIYDVSKRPSGVPDRFSG |
| | | DNAKNSLYLQMNSLRAEDTAVYYC | | SKSGNTASLTISGLQAEDEADY |
| | | ARDGGITGRYFDLWGRGTLVTVSS | | YCCSYAGSYTWVFGGGTKLTVL |
| clonotype | 855 | EVQLVESGGGLVKPGGSLRLSCAA | 991 | QSALTQPASVSGSPGQSITISC |
| 282 | | SGFTFSSYSMNWVRQAPGKGLEWV | | TGTSSDVGGYNYVSWYQQHPGK |
| | | SSISSSSSYIYYADSVKGRFTISR | | APKLMIYDVSKRPSGVSNRFSG |
| | | DNAKNSLYLQMNSLRAEDTAVYYC | | SKSGNTASLTISGLQAEDEADY |
| | | AREGNWGPYYFDYWGQGTLVTVSS | | YCCSYAGSSTVVFGGGTKLTVL |
| clonotype | 856 | QVQLVQSGAEVKKPGASVKVSCKA | 992 | QSALTQPPSASGSPGQSVTISC |
| 283 | | SGYTFTGYYMHWVRQAPGQGLEWM | | TGTSSDVGGYNYVSWYQQHPGK |
| | | GWINPNSGGTNYAQKFQGRVTMTR | | APKLMIYEVSKRPSGVPDRFSG |
| | | DTSISTAYMELSRLRSDDTAVYYC | | SKSGNTASLTVSGLQAEDEADY |
| | | ARGVWSGYYTFDPWGQGTLVTVSS | | YCSSYAGSNNWVFGGGTKLTVL |
| clonotype | 857 | EVQLVESGGGLVKPGGSLRLSCAA | 993 | QSALTQPASVSGSPGQSITISC |
| 284 | | SGFTFSNAWMSWVRQAPGKGLEWV | | TGTSSDVGGYNYVSWYQQHPGK |
| | | GRIKSKTDGGTTDYAAPVKGRFTI | | APKLMIYEVSNRPSGVSNRFSG |
| | | SRDDSKNTLYLQMNSLKTEDTAVY | | SKSGNTASLTISGLQAEDEADY |
| | | YCTPHSSSPVFDYWGQGTLVTVSS | | YCSSYTSSSHVVFGGGTKLTVL |
| clonotype | 858 | QVQLVQSGAEVKKPGASVKVSCKA | 994 | SYELTQPPSVSVSPGQTASITC |
| 286 | | SGYTFTGYYMHWVRQAPGQGLEWM | | SGDKLGDKYACWYQQKPGQSPV |
| | | GWINPNSGGTNYAQKFQGRVTMTR | | LVIYQDSKRPSGIPERFSGSNS |
| | | DTSISTAYMELSRLRSDDTAVYYC | | GNTATLTISGTQAMDEADYYCQ |
| | | ARDDTGTTGGYFQHWGQGTLVTVS | | AWDSSTVVFGGGTKLTVL |
| | | S | | |
| clonotype | 859 | QVQLVQSGAEVKKPGASVKVSCKA | 995 | SYELTQPPSVSVSPGQTASITC |
| 287 | | SGYTFTSYDINWVRQATGQGLEWM | | SGDKLGDKYACWYQQKPGQSPV |
| | | GWMNPNSGNTGYAQKFQGRVTMTR | | LVIYQDSKRPSGIPERFSGSNS |
| | | NTSISTAYMELSSLRSEDTAVYYC | | GNTATLTISGTQAMDEADYYCQ |
| | | ARAVAVAGTGWFDPWGQGTLVTVS | | AWDSSTVVFGGGTKLTVL |
| | | S | | |
| clonotype | 860 | EVQLVESGGGLVKPGGSLRLSCAA | 996 | SYELTQPSSVSVSPGQTARITC |
| 288 | | SGFTFSNAWMSWVRQAPGKGLEWV | | SGDVLAKKYARWFQQKPGQAPV |
| | | GRIKSKTDGGTTDYAAPVKGRFTI | | LVIYKDSERPSGIPERFSGSSS |
| | | SRDDSKNTLYLQMNSLKTEDTAVY | | GTTVTLTISGAQVEDEADYYCY |
| | | YCTTNYGDYVGFDYWGQGTLVTVS | | SAADNNLVFGGGTKLTVL |
| | | S | | |
| clonotype | 861 | EVHLVESGGGLVQPGGSLRLSCAA | | SYELTQPPSVSVSPGQTARITC |
| 289 | | SGFTFSGYWMSWVRQAPGKGLEWV | | SGDALPQKYAFWYQQKSGQAPV |
| | | ANIKQDGSDKYYVDSVKGRFTISR | | LVIYEDSERPSGIPERFSGSTS |
| | | DNAINSLFLQLTSLRAEDTAVYYC | | GTMATLTISGAQVEDEADYYCF |
| | | AREIDWNYGFHFDYWGQGTLITVS | | STDSSGNKVFGGGTKLTVL |
| | | S | | |
| clonotype | 862 | QVQLVQSGAEVKKPGASVKVSCKA | 998 | SYELTQPPSVSVSPGQTARITC |
| 290 | | SGYTFTSYDINWVRQATGQGLEWM | | SGDALPKKYAYWYQQKSGQAPV |
| | | GWMNPNSGNTGYAQKFQGRVTMTR | | LVIYEDSKRPSGIPERFSGSSS |
| | | NTSISTAYMELSSLRSEDTAVYYC | | GTMATLTISGAQVEDEADYYCY |
| | | ARGYYDFWSGPFDYWGQGTLVTVS | | STDSSGNRVFGGGTKLTVL |
| | | S | | |
| clonotype | 863 | QVQLVESGGGVVQPGRSLRLSCAA | 999 | SYELTQPPSVSVSPGQTARITC |
| 291 | | SGFTFSSYGMHWVRQAPGKGLEWV | | SGDALPKKYAYWYQQKSGQAPV |
| | | AVIWYDGSNKYYADSVKGRFTISR | | LVIYEDSKRPSGIPERFSGSSS |
| | | DNSKNTLYLQMNSLRAEDTAVYYC | | GTMATLTISGAQVEDEADYYCY |
| | | ARDSKWELLNWFDPWGQGTLVTVS | | STDSSGNRVFGGGTKLTVL |
| | | S | | |
| clonotype | 864 | QVQLQESGPGLVKPSETLSLTCTV | 1000 | SSELTQDPAVSVALGQTVRITC |
| 292 | | SGGSISDYYWNWIRQPPGKGLEWI | | QGDSLRSYYASWYQQKPGQAPV |
| | | GYISSRGRTNYNPSLKSRVTLSVD | | LVIYGKNNRPSGIPDRFSGSSS |
| | | SSKNQFSLKLTSVTAADTAVFYCA | | GNTASLTITGAQAEDEADYYCN |
| | | RGRHFDWLLSYFDYWGQGTLVTVS | | SRDSSGNHYVFGTGTKVTVL |
| | | S | | |
| clonotype | 865 | EVQLVESGGGLVKPGGSLRLSCAA | 1001 | SSELTQDPAVSVALGQTVRITC |
| 293 | | SGFTFSSDNMNWVRQAPGKGLEWV | | QGDSLRNYYASWYQQKPGQAPI |
| | | SSIGSSSSYIYYADSVKGRFTISR | | LVIYGKNNRPSGIPDRFSGSSS |
| | | DNAKNSLYLQMNSLRAEDTAVYYC | | GNTASLTITGAQAEDEADYYCN |
| | | ARDRAIVGATWFDPWGQGTLVIVS | | SRDSSYNHWVFGGGTKLTVL |
| | | S | | |
| clonotype | 866 | EVQLVESGGGLVKPGGSLRLSCAA | 1002 | SSELTQDPAVSVALGQTVRITC |
| 294 | | SGFTFSSYSMNWVRQAPGKGLEWV | | QGDSLRSYYASWYQQKPGQAPV |
| | | SSISSSSSYIYYADSVKGRFTISR | | LVIYGKNNRPSGIPDRFSGSSS |
| | | DNAKNSLYLQMNSLRAEDTAVYYC | | GNTASLTITGAQAEDEADYYCN |
| | | ARDRYNWNYRYFDLWGRGTLVTVS | | SRDSSGNHLVFGGGTKLTVL |
| | | S | | |
| clonotype | 867 | EVQLVESGGGLVKPGGSLRLSCAA | 1003 | SYELTQPPSVSVSPGQTARITC |
| 295 | | SGFTFSSYSMNWVRQAPGKGLEWV | | SGDALPKKYAYWYQQKSGQAPV |
| | | SSISSSSSYIYYADSVKGRFTISR | | LVIYEDSKRPSGIPERFSGSSS |
| | | DNAKNSLYLQMNSLRAEDTAVYYC | | GTMATLTISGAQVEDEADYYCY |
| | | ARDSHDYGDSYFDYWGQGTLVTVS | | STDSSGNHRVFGGGTKLTVL |
| | | S | | |
| clonotype | 868 | QVQLVQSGAEVKKPGASVKVSCKA | 1004 | SSELTQDPAVSVALGQTVRITC |
| 296 | | SGYTFTSYGISWVRQAPGQGLEWM | | QGDSLRSYYASWYQQKPGQAPV |
| | | GWISAYNGNTNYAQKLQGRVTMTT | | LVIYGKNNRPSGIPDRFSGSSS |
| | | DTSTSTAYMELRSLRSDDTAVYYC | | GNTASLTITGAQAEDEADYYCN |
| | | ARDGAARPPRYMDVWGKGTTVTVS | | SRDSSGNHLVFGGGTKLTVL |
| | | S | | |
| clonotype | 869 | QVQLVQSGAEVRKPVASVKVSCKA | 1005 | SSELTQDPAVSVALGQTVRITC |
| 297 | | SGYTFTDHSIHWVRQAPGQGLEWM | | QGDSLRSYYASWYQQKPGQAPV |
| | | GSINPNSGGTNYAQKFQGRVTMTR | | LVIYGKNNRPSGIPDRFSGSSS |
| | | DTYNCTAYMELSRLRSDDTAVYYC | | GNTASLTITGAQAEDEADYYCN |
| | | ARSDSGSHYVFFDDWGQGTLVTVS | | SRDSSGNHWVFGGGTKLTVL |
| | | S | | |
| clonotype | 870 | QVQLVQSGSEVKKPGASVKVSCKA | 1006 | SSELTQDPAVSVALGQTVRITC |
| 298 | | SGYTFTGYYMYWVRQAPGQGLEWM | | QGDSLRSYYASWYQQKPGQAPV |
| | | GWINPNSGGTNYAQKFQDRVTMTR | | LVIYGKNNRPSGIPDRFSGSSS |
| | | DTSISTAYMELSRLRSDDTAIYYC | | GNTASLTITGAQAEDEADYYCN |
| | | ARDLDYYGSGNYDYWGQGTLVTVS | | SRDSSDNHRVFGGGTKLTVL |
| | | S | | |
| clonotype | 871 | EVQLVESGGGLVQPGGSLRLSCAA | 1007 | SSELTQDPAVSVALGQTVRITC |
| 300 | | SGFTFSSYWMHWVRQAPGKGLVWV | | QGDSLRSYYASWYQQKPGQAPV |
| | | SRVNSDGSNTTYADSVKGRFTISR | | LVIYGQNNRPSGIPDRESGSSS |
| | | DNAKNTLYLQMNSLRAEDTAVYYC | | GNTASLTITGAQAEDEADYYCN |
| | | ARNRDYHGSGSFDYWGQGTLVTVS | | SRDSSGNHWVFGGGTKLTVL |
| | | S | | |
| clonotype | 872 | QVQLQQSGPGLVKPSQTLSLTCAI | 1008 | SSELTQDPAVSVALGQTVRITC |
| 301 | | SGDNVSSNSAAWNWIRQSPSRGLE | | QGDSLRSYYASWYQQKPGQAPV |
| | | WLGRTYYRSKWYNDYAVSVKSRIT | | LVIYGKNNRPSGIPDRFSGSSS |
| | | INPDTSKNQFSLHLNSVTPEDTAL | | GNTASLTITGAQAEDEADYYCN |
| | | YYCARDWNFAFDIWGQGTMVTVSS | | SRDSSGNHLVFGGGTKLTVL |
| clonotype | 873 | QVQLVQSGAEVKKPGASVKVSCKA | 1009 | QSVLSQPPSVSEAPRQRVTISC |
| 302 | | SGYTFTSYGITWVRQAPGQGLEWM | | SGSSSNIGYNAVNWYQQLPGKA |
| | | GWISAYNGNTHYAQKLQGRVTMTT | | PKLLISHDDLLPSGVSDRESGS |
| | | DTSTSTAYMDLRSLRSDDTAVYYC | | KSGTSASLAISGLQSDDEADYY |
| | | ARTIFGVVNNWFDPWGQGTLVTVS | | CAAWDARLNGWVFGGGTKLTVL |
| | | S | | |
| clonotype | 874 | QVQLVQSGAEVKKPGASVKVSCKA | 1010 | QSALTQPRSVSGSPGQSVTISC |
| 303 | | SGYTFTGYYMHWVRQAPGQGLEWM | | TGTSSDVGGYNYVSWYQQHPGK |
| | | GWINPNSGGTNYAQKFQGRVTMTR | | APKLMIYDVSKRPSGVPDRESG |
| | | DTSISTAYMELSRLRSDDTAVYYC | | SKSGNTASLTISGLQAEDEADY |
| | | ARDGEQLALNWFDPWGQGTLVTVS | | YCCSYAGSYTWVFGGGTKLTVL |
| | | S | | |
| clonotype | 875 | EVQLVESGGGLVKPGGSLRLSCAA | 1011 | QSVLTQPPSVSGAPGQRVTISC |
| 304 | | SGFTFSRYSMNWVRQAPGKGLEWV | | TGSSSNIGARYDVHWYQLLPGS |
| | | SSIISSTSYIYYADSVKGRFTISR | | APKLLIYDNSDRPSGVPDRFSG |
| | | DNAKNSLYLQMNSLRAEDTAVYYC | | SRSGTSASLAITGLQAEDEADY |
| | | ARETPVTLFDAFDIWGQGTMVTVS | | FCQSYDSSLSGSVFGGGTKLTV |
| | | S | | L |
| clonotype | 876 | EVHLVESGGGLVQPGRSLRLSCAA | 1012 | QSVLTQPPSVSGAPGQRVTISC |
| 305 | | SGFTFDEYAMHWVRQVPGKGLEWV | | TGSSSNIGAGYDVHWYQQLPGT |
| | | SGISWNSGSIGYADSVKGRFTISR | | APKLLIYGNSNRPSGVPDRESG |
| | | DNAKNSLYLQMNSLRAEDTALYYC | | SKSGTSASLAITGLQAEDEADY |
| | | AKDIFTGRAGYFDYWGQGTLVTVS | | YCQSYDSSLSGWVFGGGTKLTV |
| | | S | | L |
| clonotype | 877 | QVQLVESGGGVVQPGRSLRLSCAA | 1013 | QSVLTQPPSVSGAPGQRVTISC |
| 306 | | SGFTFSSYGMHWVRQAPGKGLEWV | | TGSSSNIGAGYDVHWYQQLPGT |
| | | AVIWYDGSNKYYADSVKGRFTISR | | APKLLIHGNSNRPSGVPDRFSG |
| | | DNSKNTLYLQMNSLRAEDTAVYYC | | SKSGTSASLAITGLQAEDETDY |
| | | ARAITGTTGNWFDPWGQGTLVTVS | | YCQSYDSSLSGWVFGGGTKLTV |
| | | S | | L |
| clonotype | 878 | QITLKESGPTLVKPTQTLTLTCTF | 1014 | QAVLTQPASLSASPGASASLTC |
| 307 | | SGFSISTSGVGVGWIRQPPGKALE | | TLRSGINVGTSRIYWYQQKPGS |
| | | WLAFIFWNDDKRYSPSLKSRLTIT | | PPQYLLRYKSDSDKHQDSGVPS |
| | | KDTSKNQVVLTMTNMDPVDTATYY | | RFSGSKDASANAGILLISGLQS |
| | | CTHTEYGSSWSVDYWGQGTLVTVS | | EDEADYYCMIWHSSAVVFGGGT |
| | | S | | KLTVL |
| clonotype | 879 | EVQLVESGGGVVRPGGSLRLSCAA | 1015 | QAVLTQPASLSASPGASASLTC |
| 308 | | SGFTEDDYGMSWVRQAPGKGLEWV | | TLRSGINVGTYRIYWYQQKPGS |
| | | SGINWNGGSTGYADSVKGRFTISR | | PPQYLLRYKSDSDKQQGSGVPS |
| | | DNAKNSLYLQMNSLRAEDTALYYC | | RFSGSKDASANAGILLISGLQS |
| | | ARHFDWLLSNAFDIWGQGTMVTVS | | EDEADYYCMIWHSSASVVFGGG |
| | | S | | TKLTVL |
| clonotype | 880 | QVQLVQSGAEVKKPGASVKVSCKA | 1016 | SYELTQPPSVSVSPGQTAIITC |
| 309 | | SGYTFTSYDINWVRQATGQGLEWM | | SGAKLGDKYACWYQKKPGQSPV |
| | | GWMNPNSGNTGYAQKFQGRVTMTR | | MVIYQDRKRPSGIPERFSGSNS |
| | | NTSISTAYMELSSLRSEDTAVYYC | | GNTATLTISGTQAMDEADYYCQ |
| | | VRRITVVRGVISLDYWGQGTLVTV | | AWDSSTAVFGGGTKLTVL |
| | | SS | | |
| clonotype | 881 | EVQLVESGGGLVKPGGSLRLSCAA | 1017 | SYELTQPPSVSVSPGQTARITC |
| 310 | | SGFTFSSYSMNWVRQAPGKGLEWV | | SGDALPKKYAYWYQQKSGQAPV |
| | | SSISSSSSYIYYADSVKGRFTISR | | LVIYEDSKRPSGIPERFSGSSS |
| | | DNAKNSLYLQMNSLRAEDTAVYYC | | GTMATLTISGAQVEDEADYYCY |
| | | ARETYYYDSSGYFDYWGQGTLVTV | | STDSSGNHRVFGGGTKLTVL |
| | | SS | | |
| clonotype | 882 | EVQLVESGGGLVQPGGSLRLSCAA | 1018 | SSELTQDPAVSVALGQTVRITC |
| 316 | | SGFTFSSYWMSWVRQAPGKGLEWV | | QGDSLRSYYASWYQQKPGQAPV |
| | | ANIKQDGSEKYYVDSVKGRFTISR | | LVIYGKNNRPSGIPDRFSGSSS |
| | | DNAKNSLYLQMNSLRAEDTAVYYC | | GNTASLTITGAQAEDEADYYCN |
| | | ARDDTIFGVVTDAFDIWGQGTMVT | | SRDSSGNLFGGGTKLTVL |
| | | VSS | | |
| clonotype | 883 | QVQLQQSGPGLVKPSQTLSLTCAI | 1019 | SYELTQPPSVSVSPGQTASITC |
| 318 | | SGDSVSSNSAAWNWIRQSPSRGLE | | SGDKLGDKYACWYQQKPGQSPV |
| | | WLGRTYYRSKWYNDYAVSVKSRIT | | LVIYQDSKRPSGIPERISGSNS |
| | | INPDTSKNQFSLQLNSVTPEDTAV | | GNTATLTISGTQAMDEADYYCQ |
| | | YYCARGVGARGWFDPWGQGTLVTV | | AWDSSTAVFGGGTKLTVL |
| | | SS | | |
| clonotype | 884 | EVQLVESGGGLVKPGGSLRLSCAA | 1020 | SSELTQDPAVSVALGQTVRITC |
| 319 | | SGFTFSTYSMNWVRQAPGKGLEWV | | QGDSLRSYYASWYQQKPGQAPV |
| | | SSISSSSTYIYYADSVKGRFTISR | | LVIYGKNNRPSGIPDRFSGSSS |
| | | DNAKNSLYLQMNSLRADDTAVYYC | | GNTASLTITGAQAEDEADYYCN |
| | | ARDPPLSGSYAGEFDYWGQGTLVT | | SRDSSGNHWVFGGGTKLTVL |
| | | VSS | | |
| clonotype | 885 | QVTLRESGPALVKPTQTLTLTCTF | 1021 | SYELTQPPSVSVSPGQTARITC |
| 320 | | SGFSLSTSGMCVSWIRQPPGKALE | | SGDALPKKYAYWYQQKSGQAPV |
| | | WLALIDWDDDKYYSTSLKTRLTIS | | LVIYEDSKRPSGIPERFSGSSS |
| | | KDTSKNQVVLTMTNMDPVDTATYY | | GTMATLTISGAQVEDEADYYCY |
| | | CARRRGYSYGWGDFDYWGQGTLVT | | STDSSGNHRVFGGGTKLTVL |
| | | VSS | | |
| clonotype | 886 | EVQLVESGGGLVQPGGSLRLSCAA | 1022 | SYELTQPPSVSVSPGQTARITC |
| 322 | | SEFIFRSYNMNWVRQAPGKGLEWV | | SGDALPKKYAYWYQQKSGQAPV |
| | | SYISISSRTIYYADSVKGRFTISR | | LVIYEDSKRPSGIPERFSGSSS |
| | | DNAKNSLFLQMNSLRDEDTAVYYC | | GTMATLTISGAQVEDEADYYCY |
| | | ARGGLLNWNYEGWFDPWGQGTLVT | | STDSSGNHRVFGGGTKLTVL |
| | | VSS | | |
| clonotype | 887 | QVQLVESGGGLVKPGGSLRLSCAA | 1023 | SYELTQPPSVSVSPGQTARITC |
| 323 | | SGFTFSDYYMSWIRQAPGKGLEWV | | SGDALPKKYAYWYQQKSGQAPV |
| | | SYISSSGSTIYYADSVKGRFTISR | | LVIYEDSKRPSGIPERFSGSSS |
| | | DNAKNSLYLQMNSLRAEDTAVYYC | | GTMATLTISGAQVEDEADYYCY |
| | | ARDGGIAARPDWYFDLWGRGTLVT | | STDSSGNHRVFGGGTKLTVL |
| | | VSS | | |
| clonotype | 888 | EVQLVESGGGLVQPGGSLRLSCAA | 1024 | QSVLTQPPSVSGAPGQRVTISC |
| 326 | | SGFTFSSYSMNWVRQAPGKGLEWV | | TGSSSNIGAGYDVHWYQQLPGT |
| | | SYISSSSSTIYYADSVKGRFTISR | | APKLLIYGNSNRPSGVPDRFSG |
| | | DNAKNSLYLQMNSLRDEDTAVYYC | | SKSGTSASLAITGLQAEDEADY |
| | | ARTYYYGSGSYYTLDYWGQGTLVT | | YCQSYDSSLSGVVFGGGTKLTV |
| | | VSS | | L |
| clonotype | 889 | QVQLVQSGAEVKKPGASVKVSCKA | 1025 | QAVLTQPASLSASPGASASLTC |
| 327 | | SGYTFTSYDINWVRQATGQGLEWM | | TLRSGINVGTYRIYWYQQKPGS |
| | | GWMNPNSGNTGYAQKFQGRVTMTR | | PPQYLLRYKSDSDKQQGSGVPS |
| | | NTSISTAYMELSSLRSEDTAVYYC | | RFSGSKDASANAGILLISGLQS |
| | | ARGGITIFGVVTPFDYWGQGTLVT | | EDEADYYCMIWHSSAWVFGGGT |
| | | VSS | | KLTVL |
| clonotype | 890 | QVQLQESGPGLVKPSQTLSLTCTV | 1026 | SYELTQPPSVSVSPGQTASITC |
| 328 | | SGGSISSGGYYWSWIRQHPGKGLE | | SGDKLGDKYACWYQQKPGQSPV |
| | | WIGYIYYSGSTYYNPSLKSRVTIS | | LVIYQDSKRPSGIPERFSGSNS |
| | | VDTSKNQFSLKLSSVTAADTAVYY | | GNTATLTISGTQAMDEADYYCQ |
| | | CARDALHYYGSGSAFDYWGQGTLV | | AWDSSTVVFGGGTKLTVL |
| | | TVSS | | |
| clonotype | 891 | EVQLVESGGGLVQPGGSLRLSCAA | 1027 | SSELTQDPAVSVALGQTVRITC |
| 333 | | SGFTFSSYWMSWVRQAPGKGLEWV | | QGDSLRRYYASWYQQKPGQAPV |
| | | ANIKQDGSEKYYVDSVKGRFTISR | | LVIYGKNNRPSGIPDRFSGSSS |
| | | DNAKNSLYLQMNSLRAEDTAVYYC | | GNTASLTITGAQAEDEADYYCN |
| | | AREGVLWFGEFYYYMDVWGKGTTV | | SRDSSGNHLVFGGGTKLTVL |
| | | TVSS | | |
| clonotype | 892 | EVQLVESGGGLVQPGGSLRLSCAA | 1028 | SYELTQPPSVSVSPGQTARITC |
| 339 | | SGFTFSSYSMNWVRQAPGKGLEWV | | SGDALPKKYAYWYQQKSGQAPV |
| | | SYISSSSSTIYYADSVKGRFTISR | | LVIYEDSKRPSGIPERFSGSSS |
| | | DNAKNSLYLQMNSLRDEDTAVYYC | | GTMATLTISGAQVEDEADYYCY |
| | | ARDGDYYDSSGYYHFDYWGQGTLV | | STDSSGNHRVFGGGTKLTVL |
| | | TVSS | | |
| clonotype | 893 | EVQLVESGGGLVQPGGSLRLSCAA | 1029 | SYELTQPPSVSVSPGQTARITC |
| 340 | | SGFTFSSYAMSWVRQAPGKGLEWV | | SGDALPKKYAYWYQQKSGQAPV |
| | | SAISGSGGSTYYADSVKGRFTISR | | LVIYEDSKRPSGIPERFSGSSS |
| | | DNSKNTLYLQMNSLRAEDTAVYYC | | GTMATLTISGAQVEDEADYYCY |
| | | AKDRGGENWNYGGWFDPWGQGTLV | | STDSSGNHRVFGGGTKLTVL |
| | | TVSS | | |
| clonotype | 894 | QVHLVESGGGVVQPGRSLRLSCAA | 1030 | SYVLIQPPSVSVAPGKTARITC |
| 341 | | SGFTFSSYGMHWVRQAPGKGLEWV | | GGNNIGGKSVHWYQLKPGQAPV |
| | | AIIWYDGSNEYYADSVKGRFTISR | | LVICYNRDRPSGIPERFSGSNS |
| | | DNSKNTLYLQMNTLRAEDTAVYYC | | GNTATLTISRVEAGDEADYYCQ |
| | | AGAYYYDSSGYLNYMDVWGKGTTV | | VWDSSSDHPVFGGGTKLTVL |
| | | TVSS | | |
| clonotype | 895 | EVQLVESGGGLVKPGGSLRLSCAA | 1031 | QSVLTQSPSASGTPGQRVTISC |
| 342 | | SGFTFRNAWMSWVRQAPGKGLEWV | | SGSNSNIGFNTVNWYQQLPGTA |
| | | GRIKTKTDGGATQYAAPVKGRFTI | | PKLLIDSNNQRPSGVPDRESGS |
| | | SRDDSKNTLYLQMNSLKTEDTAVY | | TSGTSASLAISGLQSEDEADYY |
| | | YCTTDHIEYSSLYYFDYWGQGTLV | | CSSYAGSNNFVFGTGTKVTVL |
| | | TVSS | | |
| clonotype | 896 | QVQLVQSGAEVKKPGASVKVSCKA | 1032 | QSALTQPPSASGSPGQSVTISC |
| 343 | | SGYTFTSYGISWVRQAPGQGLEWM | | TGTSSDVGGYNYVSWYQQHPGK |
| | | GWISAYNGNTNYAQKLQGRVTMTT | | APKLMIYEVSKRPSGVPDRFSG |
| | | DTSTSTAYMELRSLRSDDTAVYYC | | SKSGNTASLTVSGLQAEDEADY |
| | | ARQLAYCGGDCYLYFDYWGQGTLV | | YCSSYAGSNNLVFGGGTKLTVL |
| | | TVSS | | |
| clonotype | 897 | QVQLQQSGPGLVKPSQTLSLTCAI | 1033 | QSALTQPPSASGSPGQSVTISC |
| 345 | | SGDSVSSNSAAWNWIRQSPSRGLE | | TGTSSDVGGYNYVSWYQQHPGK |
| | | WLGRTYYRSKWYNDYAVSVKSRIT | | APKLMIYEVSKRPSGVPDRFSG |
| | | INPDTSKNQFSLQLNSVTPEDTAV | | SKSGNTASLTVSGLQAEDEADY |
| | | YYCAREAYWNYGGFDYWGQGTLVT | | YCSSYAGSNNFGVFGGGTKLTV |
| | | VSS | | L |
| clonotype | 898 | EVQLVESGGGLVKPGGSLKLSCAA | 1034 | SYELTQPPSVSVSPGQTANITC |
| 349 | | SGFTFSSYSMNWVRQAPGKGLEWV | | SGDKLGNKYACWYQQKPGQSPV |
| | | SSISSSSSYIYYADSVKGRFTISR | | LVIFQDNKRPSGIPERFSGSNS |
| | | DNAKNSLYLQMNSLRAEDTAVYYC | | GNTATLTIGGTQAMDEADYYCQ |
| | | ARDGRITMVRGVRNWFDPWGQGTL | | AWDSSTVVFGGGTKVTVL |
| | | VTVSS | | |
| clonotype | 899 | EVQLVESGGGLVQPGGSLRLSCAA | 1035 | SYDLTQPPSVSVSPGQTASITC |
| 350 | | SGFTFSSYSMNWVRQAPGKGLEWV | | SGDKLGDKYACWYQQKPGQSPV |
| | | SYISSSTSTIYYADSVKGRFTISR | | LVIYQDIKRPSGIPERFSGSNS |
| | | DNAKNSLYLQMNSLTDEDTAVYYC | | GNTATLTISGTQAMDEADYYCQ |
| | | ARMSSQLELHYYCYYMDVWGKGTT | | AWDSSTVVFGGGTKLTVL |
| | | VTVSS | | |
| clonotype | 900 | EVQLVESGGGLVKPGGSLRLSCAA | 1036 | SYELTQPPSVSVSPGQTASITC |
| 351 | | SGFTFSNAWMSWVRQAPGKGLEWV | | SGDKLGDKYACWYQQKPGQSPV |
| | | GRIKSKTDGGTTDYAAPVKGRFTI | | LVIYQDSMRPSGIPERFSGSNS |
| | | SRDDSKNTLYLQMNSLKTEDTAVY | | GNTATLTISGTQAMDEADYYCQ |
| | | YCTTDLGYSGYDWGAFDYWGQGTL | | AWDSSTVVFGGGTKLTVL |
| | | VTVSS | | |
| clonotype | 901 | QVQLQQSGPGLVKPSQTLSLTCAI | 1037 | SYELTQPPSVSVSPGQTASITC |
| 352 | | SGDSVSSNSAAWNWIRQSPSRGLE | | SGDKLGDKYACWYQQKPGQSPV |
| | | WLGRTYYRSKWYNDYAVSVKSRIT | | LVIYQDSKRPSGIPERFSGSNS |
| | | INPDTSKNQFSLQLNSVTPEDTAV | | GNTATLTISGTQAMDEADYYCQ |
| | | YYCARDRVNWNDVGFDYWGQGTLV | | AWDSSTVVFGGGTKLTVL |
| | | TVSS | | |
| clonotype | 902 | EVQLVESGGGLVQPGGSLRLSCAA | 1038 | SYELTQPPSVSVSPGQTARITC |
| 356 | | SGFTFSSYWMSWVRQAPGKGLEWV | | SGDALPKKYAYWYQQKSGQAPV |
| | | ANIKQDGSEKYYVDSVKGRFTISR | | LVIYEDSKRPSGIPERFSGSSS |
| | | DNAKNSLYLQMNSLRAEDTAVYYC | | GTMATLTISGAQVEDEADYYCY |
| | | ARTPGYSSSWYEGPYFDYWGQGTL | | STDSSGNHVVFGGGTKLTVL |
| | | VTVSS | | |
| clonotype | 903 | QLQLQESGPGLVKPSETLSLTCTV | 1039 | SYELTQPPSVSVSPGQTARITC |
| 358 | | SGGSITTRSYYWGWLRQPPGKGLE | | SGDALPKKYAYWYQQKSGQAPV |
| | | WIGTFYYSGNTYYNPSLQSRVSIS | | LVIYEDSKRPSGIPERFSGSSS |
| | | VDASKNQFSLQLSSVTAADTAVFY | | GTMATLTISGAQVEDEADYYCY |
| | | CAREDLIGNDYWGQGTLVTVSS | | STDSSGNHRVFGGGTKLTVL |
| clonotype | 904 | QLQLQESGPGLVKPSETLSLTCTV | 1040 | QSVLTQPPSASGTPGQRVTISC |
| 359 | | SGGSISTRSYYWGWLRQPPGKGLE | | SGSSSNIGINTVNWYQQVPGTA |
| | | WIGTFYYSGSTYYNPSLKSRVSIS | | PKLLIYFNNQRPSGVPDRESGS |
| | | VDTSKNQFSLQLSSVTAADTAVYY | | KSGTSASLAISGLQSEDEADYY |
| | | CAREDLIGNDYWGQGTLVTVSS | | CAAWDDSLKVFGGGTKLTVL |
| clonotype | 905 | QVQLQQWGAGLLKPSETLSLTCAV | 1041 | QTVVTQEPSLTVSPGGTVTLTC |
| 360 | | YGGSFSGHYWNWIRQPPGKGLEWI | | ASSTGAVTSGYYPNWFQQKPGQ |
| | | GEINHSGFTNYNPSLKSRVTISVD | | APRALIYSTSNKHSWTPARFSG |
| | | TPKNQFPLNLSSVTAADTAVYYCA | | SLLGGKAALTLSGVQPEDEAEY |
| | | REGLTGHVFDIWGQGTMVTVSS | | YCLLYYGGAQVFGGGTKLTVL |
| clonotype | 906 | QVQLQQWGAGLLKPSETLSLTCAV | 1042 | QSALTQPASVSGSPGQSITISC |
| 361 | | YGGFLRGYYWSWIRQPPGKGLEWI | | TGTSSDVGGYNYVSWYQQHPGK |
| | | GEINHSGSTNYNPSLKSRVTISVD | | APKLMIYDVSKRPSGVSNRFSG |
| | | TAENQFSLKLNSVTAADTAVYYCA | | SKSGNTASLTISGLQAEDEADY |
| | | REGLTGHTFDIWGQGTMVTVSS | | YCCSYAGSSTVVFGGGTKLTVL |
| clonotype | 907 | QVQLVQSGAEVKKPGASVKVSCKA | 1043 | SYELTQPSSVSVSPGQAARITC |
| 363 | | SGYIFSNYGICWVRQAPGQGLEWM | | SGNLLAKKYPRWFLQKPGQAPI |
| | | GWINPYNVNRNYAQSLQGRVTMTT | | MLTHTDCERPSGIPERFSGSSS |
| | | DTSTNSAYMELRSLKSDDTAVYFC | | GTTVTLTISGAQVEDEADYYCF |
| | | ARGVWGSYRSHSYYTFMDVWGKGT | | SAADNTSVFDGGTNLTVL |
| | | TVTVSS | | |
| clonotype | 908 | EVQLVESGGGLVKPGGSLRLSCAA | 1044 | SSELTQDPAVSVALGQTVRITC |
| 364 | | SGFTFSSYSMNWVRQAPGKGLEWV | | QGDSLRSYYASWYQQKPGQAPV |
| | | SSISSSSSYIYYADSVKGRFTISR | | LVIYGKNNRPSGIPDRFSGSSS |
| | | DNAKNSLYLQMNSLRAEDTAVYYC | | GNTASLTITGAQAEDEADYYCN |
| | | ARDYRPYYDILTGYSHFDYWGQGT | | SRDSSGNHVVFGGGTKLTVL |
| | | LVTVSS | | |
| clonotype | 909 | QVQLVQSGAEVKKPGASVKVSCKA | 1045 | SYELTQPPSVSVSPGQTARITC |
| 365 | | SGFTFTSYYIHWVRQAPGQGLEWM | | SGDALPKKYAYWFQQKSGQAPV |
| | | GIITPSGGTTSYAQKFQGRVTMTR | | LVIYEDSKRPSGIPERFSGSSS |
| | | DTSTSTVYMELSSLRSEDTAVYYC | | GTMATLIISGAQVEDEADYYCY |
| | | ARRVLWFGELRDYFYYMDVWGKGT | | STDSSGNHVVFGGGTKLTVL |
| | | TVTISS | | |
| clonotype | 910 | QMQLQESGPGLVRPSETLSLTCTV | 1046 | QSALTQPPSASGSPGQSVTISC |
| 366 | | SGGSISTRSYYWGWIRQPPGKGLE | | TGTSSDVGGYNYVSWYQQHPGK |
| | | WIGSVFYSGSTYYNPSLKSRVAIS | | APKLMIYEVSKRPSGVPDRFSG |
| | | VDTSKNQFSLKVNSVTAADTAVFY | | SKSGNTASLTVSGLQAEDEADY |
| | | CVRQGYDSWTGYSFFYFDYWGQGT | | YCSSYAGSNNLVFGGGTKLTVL |
| | | LVTVSS | | |
| clonotype | 911 | QVQLQQWGAGLLKPSETLSLTCAV | 1047 | QSVLNQPPSASGTPGQRVTISC |
| 367 | | YGGSFSVYYWSWIRQPPGKGLEWI | | SGSSSNIGSKTVNWYQQVPGTA |
| | | GEINHSGSTNYNPSLKSRVTISVD | | PKLLIYSSNQRPSGVPDRESGS |
| | | TSKNQFSLKLSSVTAADTAVYYCA | | KSGTSASLAISELQSEDEADYY |
| | | RGGGYSFGGFDYWGQGTLVTVSS | | CTSWDDSLNTWVFGGGTKLTVL |
| clonotype | 912 | QLQLQESGPGLVKPSETLALTCTV | 1048 | SYELTQPLSLSVALGQTARITC |
| 368 | | SGGSISSIIYYWGWIRQPPGKGLE | | GENNIGSRNVHWYQQKPGQAPV |
| | | WIGNVYYSGSIYYNPSLKSRVTIS | | LVIYRDSDRPSGIPERFSGSNS |
| | | VDTSKNQFSLKLSSVTAADTAVYY | | GNTATLTISRAQAGDEADYFCQ |
| | | CARQNWGSDAFDIWGQGTMVTVSS | | VWDSSTAVFGGGTKLTVL |
| clonotype | 913 | QVQLQQWGAGLLKPSETLSLTCAV | 1049 | SSELTQDPAVSVALGQTVRITC |
| 369 | | YGGSFSGYYWSWIRQPPGKGLEWI | | QGDSLRSYYASWYQQKPGQAPV |
| | | GEINHSGNTNYNPSLKSRVTISVD | | LVIYGKNNRPSGIPDRFSGSSS |
| | | TSKNQFSLKLSSVTAADTAVYYCA | | GNTASLTITGAQAEDEADYYCN |
| | | RGELGIGYWYFDLWGRGTLVTVSS | | SRDSSGNHVVFGGGTKLTVL |
| clonotype | 914 | QVQLQQWGAGLLKPSETLSLTCAV | 1050 | SYELTQPPSVSVSPGQTARITC |
| 370 | | YGGSFSGYYWSWIRQPPGKGLEWI | | SGDALPKKYAYWYQQKSGQAPV |
| | | GEINHSGSTNYNPSLKSRVTISVD | | LVIYEDSKRPSGIPERFSGSSS |
| | | TSKNQFSLKLSSVTAADTAVYYCA | | GTMATLTISGAQVEDEADYYCY |
| | | REGGTTHEPLFDYWGQGTLVTVSS | | STDSSGNHRVFGGGTKLTVL |
| clonotype | 915 | QVQLVQSGAEVKKPGASMKVSCKA | 1051 | RSALTQPASVSGSPGQSITISC |
| 379 | | SGYTFITYGITWVRQAPGQGLEWM | | TGTSSDVGGYNYVSWYHQHPGK |
| | | GWISAYNGNANYAQKVQDRVTMTT | | APKLMIYDVSKRPSGVSNRFSG |
| | | DTSTSTAYMELRSLRSDDTAVYYC | | FKSGNTASLTISGLQAEDEADY |
| | | ARAPGGSCGSTNCYKWNYDPYYFD | | FCCSYAGSSTLVFGGGTKLTVL |
| | | YWGQGTLVTVSS | | |
| clonotype | 916 | QVQLVQSGAEVKKPGASVKVSCKA | 1052 | QSALTQPASVSGSPGQSITISC |
| 380 | | SGYTFITYGISWVRQAPGQGLEWM | | TGTSSDVGGYNHVSWYQQNPGK |
| | | GWISSYNGNTNYAQKLQGRVTMTR | | APKLMIYDVSKRPSGVSNRFSG |
| | | DTSTSTAYMELRSLRSDDTAVYYC | | SKSGNTASLTISGLQAEDEADY |
| | | ARAPGGDCSSTSCYKWNYDPYYFD | | YCCSYAGSSTLVFGGGTKLTVL |
| | | YWGQGTLVTVSS | | |

TABLE 9
VH-CDR and VL-CDR Sequences of Antibodies of Table 8

| clono | SEQ | HCD | SEQ | HCD | SEQ | HCD | SEQ | LCD | SEQ | LCD | SEQ | LCD |
|---|---|---|---|---|---|---|---|---|---|---|---|---|
| type | ID | R1 | ID | R2 | ID | R3 | ID | R1 | ID | R2 | ID | R3 |
| _id | NO | AA | NO | AA | NO | AA | NO | AA | NO | AA | NO | AA |
| clono | 2989 | GYTF | 3125 | NPNS | 3261 | CARW | 3397 | SGDK | 3533 | QDSK | 3669 | CQAW |
| type7 | | TSY | | GN | | GRGF | | LGDK | | RPS | | DSST |
| | | | | | | DPW | | YAC | | | | AVF |
| clono | 2990 | GGTF | 3126 | IPIF | 3262 | CARE | 3398 | TGTS | 3534 | DVSK | 3670 | CCSY |
| type9 | | SSY | | GT | | RLEL | | SDVG | | RPS | | AGSS |
| | | | | | | RWFD | | GYNY | | | | TWVF |
| | | | | | | PW | | VS | | | | |
| clono | 2991 | GGSI | 3127 | YYSG | 3263 | CARQ | 3399 | TGTS | 3535 | DVSK | 3671 | CCSY |
| type14 | | SSSS | | S | | TDYW | | SDVG | | RPS | | AGSS |
| | | Y | | | | FDPW | | GYNY | | | | TLVF |
| | | | | | | | | VS | | | | |
| clono | 2992 | GFTF | 3128 | SSSS | 3264 | CARE | 3400 | TGTS | 3536 | EVSN | 3672 | CSSY |
| type15 | | SSY | | TY | | GGIA | | SDVG | | RPS | | TSSS |
| | | | | | | VAGF | | GYNY | | | | TLVF |
| | | | | | | DYW | | VS | | | | |
| clono | 2993 | GFTF | 3129 | GTAG | 3265 | CARD | 3401 | GGNN | 3537 | RDSN | 3673 | CQVW |
| typel7 | | SSY | | D | | SSSW | | IGSK | | RPS | | DSST |
| | | | | | | YEDA | | NVH | | | | VVF |
| | | | | | | FDIW | | | | | | |
| clono | 2994 | GFTF | 3130 | SSSG | 3266 | CARE | 3402 | TGTS | 3538 | EVSN | 3674 | CSSY |
| type19 | | SDY | | ST | | ETMV | | SDVG | | RPS | | TSSS |
| | | | | | | RGVI | | GYNY | | | | TVVF |
| | | | | | | AYW | | VS | | | | |
| clono | 2995 | GFTF | 3131 | SGSS | 3267 | CARD | 3403 | SGDA | 3539 | EDSK | 3675 | CYST |
| type22 | | STD | | SY | | WGSF | | LPKK | | RPS | | DSSG |
| | | | | | | DLW | | YAY | | | | NHWV |
| | | | | | | | | | | | | F |
| clono | 2996 | GFTV | 3132 | YSGG | 3268 | CARD | 3404 | TGTS | 3540 | EVSK | 3676 | CSSY |
| type29 | | SSN | | S | | GGEY | | SDVG | | RPS | | AGSN |
| | | | | | | SSSY | | GYNY | | | | NVVF |
| | | | | | | YFDY | | VS | | | | |
| | | | | | | W | | | | | | |
| clono | 2997 | GGSI | 3133 | YYSG | 3269 | CARH | 3405 | TLSS | 3541 | VKSD | 3677 | CGES |
| type32 | | SSSS | | S | | DPSF | | EHST | | GSHS | | HTID |
| | | Y | | | | DYW | | YTIE | | KGD | | GQVG |
| | | | | | | | | | | | | VVF |
| clono | 2998 | GYTF | 3134 | SAYN | 3270 | CARE | 3406 | GGNN | 3542 | YDSD | 3678 | CQVW |
| type38 | | TSY | | GN | | RSNW | | IGSK | | RPS | | DSSS |
| | | | | | | DFDY | | SVH | | | | DHRV |
| | | | | | | W | | | | | | F |
| clono | 2999 | GFTF | 3135 | KQDG | 3271 | CARD | 3407 | SGDK | 3543 | QDSK | 3679 | CQAW |
| type42 | | SSY | | SE | | GGVR | | LGDK | | RPS | | DSSN |
| | | | | | | GVIT | | YAC | | | | VVF |
| | | | | | | YFDY | | | | | | |
| | | | | | | W | | | | | | |
| clono | 3000 | GYTF | 3136 | NPNS | 3272 | CARG | 3408 | SGDK | 3544 | QDSK | 3680 | CQAW |
| type43 | | TSY | | GN | | RGSS | | LGDK | | RPS | | DSST |
| | | | | | | WYWY | | YAC | | | | AVF |
| | | | | | | FDLW | | | | | | |
| clono | 3001 | GFTF | 3137 | GTAG | 3273 | CARG | 3409 | GGNN | 3545 | RDSN | 3681 | CQVW |
| type44 | | SSY | | D | | SSSS | | IGSK | | RPS | | DSST |
| | | | | | | AFDI | | NVH | | | | WVF |
| | | | | | | W | | | | | | |
| clono | 3002 | GFTF | 3138 | SSSS | 3274 | CARD | 3410 | SGDV | 3546 | KDSE | 3682 | CYSA |
| type45 | | SSY | | SY | | GGIA | | LAKK | | RPS | | ADNN |
| | | | | | | AAGT | | YAR | | | | LVF |
| | | | | | | DYW | | | | | | |
| clono | 3003 | GFTF | 3139 | SSSS | 3275 | CARA | 3411 | SGDA | 3547 | EDSK | 3683 | CYST |
| type56 | | SSY | | SY | | DSLT | | LPKK | | RPS | | DSSG |
| | | | | | | GGFF | | YAY | | | | NHSW |
| | | | | | | DYW | | | | | | VF |
| clono | 3004 | GYTF | 3140 | NTNT | 3276 | CAER | 3412 | GSST | 3548 | DTSN | 3684 | CLLS |
| type57 | | ISY | | GN | | GWNY | | GAVT | | KHS | | YSGA |
| | | | | | | DYW | | SGHY | | | | WVF |
| | | | | | | | | PY | | | | |
| clono | 3005 | GFTV | 3141 | YSGG | 3277 | CARD | 3413 | TGTS | 3549 | EVSN | 3685 | CSSY |
| type65 | | SSN | | S | | STTV | | SDVG | | RPS | | TSSS |
| | | | | | | TLFD | | GYNY | | | | TYVF |
| | | | | | | YW | | VS | | | | |
| clono | 3006 | GFTF | 3142 | SSSS | 3278 | CARG | 3414 | TGTS | 3550 | EVSK | 3686 | CSSY |
| type68 | | SSY | | SY | | IAVA | | SDVG | | RPS | | AGSN |
| | | | | | | GPHA | | GYNY | | | | NFVV |
| | | | | | | FDIW | | VS | | | | F |
| clono | 3007 | GFTV | 3143 | YSGG | 3279 | CARG | 3415 | TGSS | 3551 | GNSN | 3687 | CQSY |
| type70 | | SSN | | S | | YSGS | | SNIG | | RPS | | DSSL |
| | | | | | | YAYW | | AGYD | | | | SGYV |
| | | | | | | | | VH | | | | F |
| clono | 3008 | GDSV | 3144 | YYRS | 3280 | CARK | 3416 | TGTS | 3552 | DVSK | 3688 | CCSY |
| type72 | | SSNS | | KWY | | WELR | | SDVG | | RPS | | AGRS |
| | | A | | | | DAFD | | GYNY | | | | TLGI |
| | | | | | | IW | | VS | | | | DWVF |
| clono | 3009 | GYSF | 3145 | YPGD | 3281 | CAKR | 3417 | EGDN | 3553 | FDSD | 3689 | CQVW |
| type74 | | SSY | | SD | | RMTG | | IGSE | | RPS | | DNGS |
| | | | | | | SHSW | | SVH | | | | DHVV |
| | | | | | | FDPW | | | | | | F |
| clono | 3010 | GFTF | 3146 | NSDG | 3282 | CARI | 3418 | TGTS | 3554 | EVSN | 3690 | CSSY |
| type215 | | SSY | | SS | | DYW | | SDVG | | RPS | | TSSS |
| | | | | | | | | GYNY | | | | TWVF |
| | | | | | | | | VS | | | | |
| clono | 3011 | GFTF | 3147 | KQDG | 3283 | CARD | 3419 | SGDA | 3555 | EDSK | 3691 | CYST |
| type216 | | SSY | | SE | | GGTP | | LPKK | | RPS | | DSSG |
| | | | | | | | | YAY | | | | NHRV |
| | | | | | | | | | | | | F |
| clono | 3012 | GGSI | 3148 | YYSG | 3284 | CAIV | 3420 | SGDV | 3556 | KDSE | 3692 | CYSA |
| type219 | | SSY | | S | | GARF | | LAKK | | RPS | | ADNN |
| | | | | | | DYW | | YAR | | | | LVF |
| clono | 3013 | GFTF | 3149 | KSKT | 3285 | CTTG | 3421 | SADA | 3557 | KDSE | 3693 | CQSA |
| type221 | | INA | | DGGT | | GTHW | | LPNQ | | RPS | | DSSG |
| | | | | | | | | YAY | | | | TWVF |
| clono | 3014 | GFTF | 3150 | KSKT | 3286 | CTTG | 3422 | SADA | 3558 | KDSE | 3694 | COSA |
| type223 | | SNA | | DGGT | | GYRW | | LSKQ | | RPS | | DSSG |
| | | | | | | | | YAY | | | | TNWV |
| | | | | | | | | | | | | F |
| clono | 3015 | GFTF | 3151 | KSKT | 3287 | CTTD | 3423 | SGDA | 3559 | EDSK | 3695 | CYST |
| type224 | | SNA | | DGGT | | LYYW | | LPKK | | RPS | | DSSG |
| | | | | | | | | YAY | | | | NHRV |
| | | | | | | | | | | | | F |
| clono | 3016 | GYTF | 3152 | SAYN | 3288 | CARG | 3424 | TGTS | 3560 | EVSN | 3696 | CSSY |
| type225 | | TSY | | GN | | WYFD | | SDVG | | RPS | | TSSS |
| | | | | | | YW | | GYNY | | | | TLVF |
| | | | | | | | | VS | | | | |
| clono | 3017 | GFTF | 3153 | SGGG | 3289 | CAKR | 3425 | TGTS | 3561 | EVSN | 3697 | CSSY |
| type226 | | STY | | GS | | VFFD | | SDVG | | RPS | | TISS |
| | | | | | | YW | | GYNY | | | | TWVF |
| | | | | | | | | VS | | | | |
| clono | 3018 | GFTF | 3154 | GTAG | 3290 | CARD | 3426 | SGDA | 3562 | EDSK | 3698 | CYST |
| type228 | | SSY | | D | | LGRV | | LPKK | | RPS | | DSSG |
| | | | | | | FDYW | | YAY | | | | NHRV |
| | | | | | | | | | | | | F |
| clono | 3019 | GFTF | 3155 | NQDG | 3291 | CATD | 3427 | TGTS | 3563 | GVSN | 3699 | CSSY |
| type229 | | SRY | | SE | | LNWN | | SDVG | | RPS | | TRSR |
| | | | | | | GYW | | GYDY | | | | TWVF |
| | | | | | | | | VS | | | | |
| clono | 3020 | GFTF | 3156 | GAAG | 3292 | CATG | 3428 | TLRS | 3564 | YKSD | 3700 | CMIW |
| type230 | | SRC | | D | | YNWN | | GINV | | SDKQ | | HSSA |
| | | | | | | PDYW | | GTYR | | QGS | | SVF |
| | | | | | | | | IY | | | | |
| clono | 3021 | GFTF | 3157 | WYDG | 3293 | CARD | 3429 | SGDV | 3565 | KDSE | 3701 | CYSA |
| type231 | | INY | | SN | | RSSS | | LAKK | | RPS | | ADNN |
| | | | | | | SDYW | | YAR | | | | RVF |
| clono | 3022 | GFTF | 3158 | NSDG | 3294 | CAGI | 3430 | SGDV | 3566 | KDSE | 3702 | CYSA |
| type232 | | SSY | | SS | | TGTY | | LAKK | | RPS | | ADNN |
| | | | | | | FDYW | | YAR | | | | LVF |
| clono | 3023 | GFTF | 3159 | SWNS | 3295 | CAKD | 3431 | SGDA | 3567 | EDSK | 3703 | CYST |
| type233 | | DDY | | GS | | SGYS | | LPKK | | RPS | | DSSG |
| | | | | | | PDYW | | YAY | | | | KGVF |
| clono | 3024 | GYTF | 3160 | SAYN | 3296 | CARD | 3432 | SGDA | 3568 | EDSK | 3704 | CYST |
| type234 | | TSY | | GN | | RPYY | | LPKK | | RPS | | DSSG |
| | | | | | | FDYW | | YAY | | | | NHRV |
| | | | | | | | | | | | | F |
| clono | 3025 | GYTF | 3161 | NPSG | 3297 | CARG | 3433 | QGDS | 3569 | GKNN | 3705 | CNSR |
| type235 | | TSY | | GS | | GWGT | | LRSY | | RPS | | DSSG |
| | | | | | | MDVW | | YAS | | | | NHYV |
| | | | | | | | | | | | | F |
| clono | 3026 | GFTF | 3162 | GTAG | 3298 | CARA | 3434 | QGDS | 3570 | GKNN | 3706 | CNSR |
| type236 | | SSY | | D | | WELD | | LRSY | | RPS | | DSSG |
| | | | | | | AFDI | | YAS | | | | NHVV |
| | | | | | | W | | | | | | F |
| clono | 3027 | GFTF | 3163 | SGSG | 3299 | CAKD | 3435 | SGDA | 3571 | EDSK | 3707 | CYST |
| type237 | | SSY | | GS | | NWNY | | LPKK | | RPS | | DSSG |
| | | | | | | FDYW | | YAY | | | | NHRV |
| | | | | | | | | | | | | F |
| clono | 3028 | GFTF | 3164 | WYDG | 3300 | CARV | 3436 | SGDA | 3572 | EDSK | 3708 | CYST |
| type238 | | SSY | | SN | | YNWI | | LPKK | | RPS | | DSSG |
| | | | | | | FDYW | | YAY | | | | NHRV |
| | | | | | | | | | | | | F |
| clono | 3029 | GFSF | 3165 | SGIS | 3301 | CARI | 3437 | TGTN | 3573 | DVIK | 3709 | CCSY |
| type239 | | SSH | | NY | | TVVS | | NDVG | | RPS | | AGSS |
| | | | | | | FDYW | | YYNY | | | | TWVF |
| | | | | | | | | VS | | | | |
| clono | 3030 | GFTF | 3166 | SGSG | 3302 | CAKA | 3438 | TGTS | 3574 | EVSK | 3710 | CSSY |
| type240 | | SSY | | GN | | AAGK | | SDVG | | RPS | | SGSN |
| | | | | | | GDYW | | GYNY | | | | NYVF |
| | | | | | | | | VS | | | | |
| clono | 3031 | GYTF | 3167 | NPSG | 3303 | CARG | 3439 | TGTS | 3575 | EVIY | 3711 | CTSY |
| type241 | | TSY | | GT | | DWGT | | SDVG | | RPS | | TRNN |
| | | | | | | MDVW | | NYNY | | | | TYVF |
| | | | | | | | | VS | | | | |
| clono | 3032 | GFTF | 3168 | KQDG | 3304 | CARD | 3440 | TGSS | 3576 | GNSN | 3712 | CQSY |
| type242 | | SSY | | SE | | GTGW | | SNIG | | RPS | | DSSL |
| | | | | | | FDPW | | AGYD | | | | SGWV |
| | | | | | | | | VH | | | | F |
| clono | 3033 | GYTF | 3169 | SAYN | 3305 | CARR | 3441 | TGTS | 3577 | DVSK | 3713 | CCSY |
| type243 | | TSY | | GN | | GTVV | | SDVG | | RPS | | AGSS |
| | | | | | | FDYW | | GYNY | | | | TYVV |
| | | | | | | | | VS | | | | F |
| clono | 3034 | GYTF | 3170 | SAYN | 3306 | CARP | 3442 | TGTS | 3578 | DVSK | 3714 | CCSY |
| type244 | | TNY | | GN | | LSGT | | SDVG | | RPS | | AGRS |
| | | | | | | LDNW | | GYNY | | | | TLGI |
| | | | | | | | | VS | | | | DWVF |
| clono | 3035 | GFTF | 3171 | SSSS | 3307 | CARE | 3443 | QGDS | 3579 | GKNN | 3715 | CNSR |
| type246 | | SSY | | ST | | SIAA | | LRSY | | RPS | | DSSG |
| | | | | | | LFDY | | YAS | | | | NHLV |
| | | | | | | W | | | | | | F |
| clono | 3036 | GFTF | 3172 | GTAG | 3308 | CARG | 3444 | SGEA | 3580 | KDSE | 3716 | CLSA |
| type247 | | SSY | | D | | DHSY | | LPKK | | RPS | | DSSG |
| | | | | | | GGLD | | YAY | | | | TYRV |
| | | | | | | YW | | | | | | F |
| clono | 3037 | GYTF | 3173 | IPNS | 3309 | CARG | 3445 | TGTS | 3581 | EVSH | 3717 | CSSY |
| type249 | | TSY | | GN | | DWAW | | SDVG | | RPS | | TSSS |
| | | | | | | SFDL | | GYNY | | | | SLVF |
| | | | | | | W | | VS | | | | |
| clono | 3038 | GYTF | 3174 | NPSG | 3310 | CARG | 3446 | TGTS | 3582 | EVSN | 3718 | CSSY |
| type250 | | TSY | | GS | | LRRD | | SDVG | | RPS | | TSSS |
| | | | | | | WFDP | | GYNY | | | | TWVF |
| | | | | | | W | | VS | | | | |
| clono | 3039 | GFTF | 3175 | KSKT | 3311 | CTTG | 3447 | TGTS | 3583 | DVTT | 3719 | CCSY |
| type251 | | SNA | | DGGT | | TGRS | | SDVG | | RPS | | SGSY |
| | | | | | | DYW | | GYNY | | | | TYVF |
| | | | | | | | | VS | | | | |
| clono | 3040 | GFTF | 3176 | WYDG | 3312 | CVRG | 3448 | TGTS | 3584 | EVSN | 3720 | CISY |
| type252 | | SNY | | NN | | GVGD | | SDVG | | RPS | | TNTN |
| | | | | | | GFDM | | GYNY | | | | TRVF |
| | | | | | | W | | VS | | | | |
| clono | 3041 | GFTF | 3177 | WYDG | 3313 | CASV | 3449 | TGTS | 3585 | EVSN | 3721 | CSSY |
| type253 | | SSY | | SN | | GSYG | | SDVG | | RPS | | TSSS |
| | | | | | | YFQH | | GYNY | | | | TWVE |
| | | | | | | W | | VS | | | | |
| clono | 3042 | GFTF | 3178 | NWNG | 3314 | CARK | 3450 | TGTS | 3586 | EVSK | 3722 | CSSY |
| type254 | | DDY | | GS | | GNWN | | SDVG | | RPS | | AGSN |
| | | | | | | SFDY | | GYNY | | | | NWVF |
| | | | | | | W | | VS | | | | |
| clono | 3043 | GFTF | 3179 | SSSS | 3315 | CARD | 3451 | TGTS | 3587 | EVSK | 3723 | CSSY |
| type255 | | SSY | | SY | | SAYY | | SDVG | | RPS | | AGSN |
| | | | | | | | | G | | | | N |
| | | | | | | TEDY | | YNYV | | | | FWVF |
| | | | | | | W | | S | | | | |
| clono | 3044 | GFTF | 3180 | SGSG | 3316 | CGSG | 3452 | TGTS | 3588 | EVSN | 3724 | CSSY |
| type256 | | SSY | | GS | | WYEG | | SDVG | | RPS | | TSSS |
| | | | | | | AFDY | | GYNY | | | | TYWV |
| | | | | | | W | | VS | | | | F |
| clono | 3045 | GFTF | 3181 | GTAG | 3317 | CARD | 3453 | SGDK | 3589 | QDSK | 3725 | CQAW |
| type259 | | SSY | | D | | RDSS | | LGDK | | RPS | | DSST |
| | | | | | | HDAF | | YAC | | | | VVF |
| | | | | | | DIW | | | | | | |
| clono | 3046 | GFPF | 3182 | NWNG | 3318 | CVRD | 3454 | SGDV | 3590 | KDSE | 3726 | CYSA |
| type260 | | DDF | | GT | | EIWN | | LAKK | | RPS | | ADNN |
| | | | | | | YYFD | | YAR | | | | RVF |
| | | | | | | YW | | | | | | |
| clono | 3047 | GFSI | 3183 | SSSS | 3319 | CARD | 3455 | SGDA | 3591 | EDSK | 3727 | CYST |
| type262 | | SIN | | TY | | SYDF | | LPKK | | RPS | | DSSG |
| | | | | | | HAFD | | YAY | | | | NHRV |
| | | | | | | IW | | | | | | F |
| clono | 3048 | GFTF | 3184 | NSDG | 3320 | CARV | 3456 | QGDS | 3592 | GKNN | 3728 | CNSR |
| type264 | | SSY | | SS | | GWGG | | LRSY | | RPS | | DSSG |
| | | | | | | HAFD | | YAS | | | | NHVV |
| | | | | | | IW | | | | | | F |
| clono | 3049 | GFTF | 3185 | SSSS | 3321 | CARG | 3457 | TGTS | 3593 | EVSN | 3729 | CSSY |
| type265 | | STY | | TY | | YNWN | | SDVG | | RPS | | TSSS |
| | | | | | | YVGD | | GYNY | | | | TLVE |
| | | | | | | YW | | VS | | | | |
| clono | 3050 | GFTF | 3186 | SGSG | 3322 | CAKD | 3458 | TGSS | 3594 | GNSN | 3730 | CQSY |
| type266 | | SSY | | GS | | ANWG | | SNIG | | RPS | | DSSL |
| | | | | | | YAFD | | AGYD | | | | SGSV |
| | | | | | | IW | | VH | | | | F |
| clono | 3051 | GYTF | 3187 | SGYS | 3323 | CARR | 3459 | SRDK | 3595 | QDSK | 3731 | CQAW |
| type270 | | TTY | | GY | | FIWN | | LGDK | | RPS | | DSST |
| | | | | | | YGDF | | YAC | | | | VVF |
| | | | | | | DYW | | | | | | |
| clono | 3052 | GFTF | 3188 | SRSS | 3324 | CARG | 3460 | SGDK | 3596 | QGSK | 3732 | CQAW |
| type271 | | SSY | | GT | | RLGI | | LGDR | | RPS | | DSST |
| | | | | | | EDYF | | YAC | | | | VVF |
| | | | | | | DYW | | | | | | |
| clono | 3053 | GFTF | 3189 | SSSS | 3325 | CARE | 3461 | QGDS | 3597 | GKNN | 3733 | CNSR |
| type272 | | NRY | | DT | | CYSS | | LRSY | | RPS | | DSSG |
| | | | | | | SWAF | | YAS | | | | WVF |
| | | | | | | DYW | | | | | | |
| clono | 3054 | GGSI | 3190 | YHSG | 3326 | CARD | 3462 | SGDK | 3598 | QDNK | 3734 | CQAW |
| type273 | | SSSN | | S | | VGVD | | LENK | | RPS | | DSTT |
| | | | | | | GRGF | | YTC | | | | AWVF |
| | | | | | | DYW | | | | | | |
| clono | 3055 | GFTF | 3191 | NLDG | 3327 | CARD | 3463 | QGDN | 3599 | GKNN | 3735 | CYSR |
| type274 | | SIY | | SE | | ILWS | | IR | | RPS | | DSSG |
| | | | | | | | | | | | | S |
| clono | 3056 | GFTF | 3192 | KHDG | 3328 | CARK | 3464 | QGDS | 3600 | GKDN | 3736 | CNSR |
| type275 | | SGY | | SE | | QLWL | | LRRY | | RPS | | DSSG |
| | | | | | | NWYF | | YAS | | | | NHLV |
| | | | | | | DFW | | | | | | F |
| clono | 3057 | GYTF | 3193 | SAFN | 3329 | CARE | 3465 | QGDS | 3601 | GKNN | 3737 | CNSR |
| type276 | | TTY | | GN | | NNWN | | LRSY | | RPS | | DSSG |
| | | | | | | YGWF | | YAS | | | | NHYV |
| | | | | | | DPW | | | | | | F |
| clono | 3058 | GFTF | 3194 | GTAG | 3330 | CARE | 3466 | SGDA | 3602 | EDSK | 3738 | CYST |
| type277 | | SSY | | D | | GYGD | | LPKK | | RPS | | DSSG |
| | | | | | | YPLP | | YAY | | | | NHRV |
| | | | | | | MDVW | | | | | | F |
| clono | 3059 | GFTF | 3195 | KSKT | 3331 | CTTD | 3467 | SGDA | 3603 | EDSK | 3739 | CYST |
| type278 | | SNA | | DGGT | | NWNS | | LPKK | | RPS | | DSSG |
| | | | | | | YFDY | | YAY | | | | NHRV |
| | | | | | | W | | | | | | F |
| clono | 3060 | GFTF | 3196 | SGGG | 3332 | CAGN | 3468 | SGDA | 3604 | EDSK | 3740 | CYST |
| type279 | | SSY | | GS | | SGYD | | LPKK | | RPS | | DSSG |
| | | | | | | SPYF | | YAY | | | | NHRV |
| | | | | | | DYW | | | | | | |
| clono | 3061 | GFTF | 3197 | WYDG | 3333 | CARE | 3469 | SGDA | 3605 | EDIK | 3741 | CYST |
| type280 | | SSY | | SN | | YSSS | | LPKK | | RPS | | DSSG |
| | | | | | | SDWF | | YAY | | | | NHRV |
| | | | | | | DPW | | | | | | F |
| clono | 3062 | GFTF | 3198 | SSSS | 3334 | CARD | 3470 | TGTS | 3606 | DVSK | 3742 | CCSY |
| type281 | | SSY | | SY | | GGIT | | SDVG | | RPS | | AGSY |
| | | | | | | GRYF | | GYNY | | | | TWVE |
| | | | | | | DLW | | VS | | | | |
| clono | 3063 | GFTF | 3199 | SSSS | 3335 | CARE | 3471 | TGTS | 3607 | DVSK | 3743 | CCSY |
| type282 | | SSY | | SY | | GNWG | | SDVG | | RPS | | AGSS |
| | | | | | | PYYF | | GYNY | | | | TVVF |
| | | | | | | DYW | | VS | | | | |
| clono | 3064 | GYTF | 3200 | NPNS | 3336 | CARG | 3472 | TGTS | 3608 | EVSK | 3744 | CSSY |
| type283 | | TGY | | GG | | VWSG | | SDVG | | RPS | | AGSN |
| | | | | | | YYTF | | GYNY | | | | NWVF |
| | | | | | | DPW | | VS | | | | |
| clono | 3065 | GFTF | 3201 | KSKT | 3337 | CTPH | 3473 | TGTS | 3609 | EVSN | 3745 | CSSY |
| type284 | | SNA | | DGGT | | SSSP | | SDVG | | RPS | | TSSS |
| | | | | | | VFDY | | GYNY | | | | HVVF |
| | | | | | | W | | VS | | | | |
| clono | 3066 | GYTF | 3202 | NPNS | 3338 | CARD | 3474 | SGDK | 3610 | QDSK | 3746 | CQAW |
| type286 | | TGY | | GG | | DTGT | | LGDK | | RPS | | DSST |
| | | | | | | TGGY | | YAC | | | | VVF |
| | | | | | | FQHW | | | | | | |
| clono | 3067 | GYTF | 3203 | NPNS | 3339 | CARA | 3475 | SGDK | 3611 | QDSK | 3747 | CQAW |
| type287 | | TSY | | GN | | VAVA | | LGDK | | RPS | | DSST |
| | | | | | | GTGW | | YAC | | | | VVF |
| | | | | | | FDPW | | | | | | |
| clono | 3068 | GFTF | 3204 | KSKT | 3340 | CTTN | 3476 | SGDV | 3612 | KDSE | 3748 | CYSA |
| type288 | | SNA | | DGGT | | YGDY | | LAKK | | RPS | | ADNN |
| | | | | | | VGFD | | YAR | | | | LVF |
| | | | | | | YW | | | | | | |
| clono | 3069 | GFTF | 3205 | KODG | 3341 | CARE | 3477 | SGDA | 3613 | EDSE | 3749 | CFST |
| type289 | | SGY | | SD | | IDWN | | LPQK | | RPS | | DSSG |
| | | | | | | YGFH | | YAF | | | | NKVF |
| | | | | | | FDYW | | | | | | |
| clono | 3070 | GYTF | 3206 | NPNS | 3342 | CARG | 3478 | SGDA | 3614 | EDSK | 3750 | CYST |
| type290 | | TSY | | GN | | YYDF | | LPKK | | RPS | | DSSG |
| | | | | | | WSGP | | YAY | | | | NRVF |
| | | | | | | FDYW | | | | | | |
| clono | 3071 | GFTF | 3207 | WYDG | 3343 | CARD | 3479 | SGDA | 3615 | EDSK | 3751 | CYST |
| type291 | | SSY | | SN | | SKWE | | LPKK | | RPS | | DSSG |
| | | | | | | LLNW | | YAY | | | | NRVF |
| | | | | | | FDPW | | | | | | |
| clono | 3072 | GGSI | 3208 | SSRG | 3344 | CARG | 3480 | QGDS | 3616 | GKNN | 3752 | CNSR |
| type292 | | SDY | | R | | RHFD | | LRSY | | RPS | | DSSG |
| | | | | | | WLLS | | YAS | | | | NHYV |
| | | | | | | YFDY | | | | | | F |
| | | | | | | W | | | | | | |
| clono | 3073 | GFTF | 3209 | GSSS | 3345 | CARD | 3481 | QGDS | 3617 | GKNN | 3753 | CNSR |
| type293 | | SSD | | SY | | RAIV | | LRNY | | RPS | | DSSY |
| | | | | | | GATW | | YAS | | | | NHWT |
| | | | | | | FDPW | | | | | | F |
| | | | | | | GQGT | | | | | | |
| | | | | | | LVIV | | | | | | |
| clono | 3074 | GFTF | 3210 | SSSS | 3346 | CARD | 3482 | QGDS | 3618 | GKNN | 3754 | CNSR |
| type294 | | SSY | | SY | | RYNW | | LRSY | | RPS | | DSSG |
| | | | | | | NYRY | | YAS | | | | NHLV |
| | | | | | | FDLW | | | | | | F |
| clono | 3075 | GFTF | 3211 | SSSS | 3347 | CARD | 3483 | SGDA | 3619 | EDSK | 3755 | CYST |
| type295 | | SSY | | SY | | SHDY | | LPKK | | RPS | | DSSG |
| | | | | | | GDSY | | YAY | | | | NHRV |
| | | | | | | FDYW | | | | | | F |
| clono | 3076 | GYTF | 3212 | SAYN | 3348 | CARD | 3484 | QGDS | 3620 | GKNN | 3756 | CNSR |
| type296 | | TSY | | GN | | GAAR | | LRSY | | RPS | | DSSG |
| | | | | | | PPRY | | YAS | | | | NHLV |
| | | | | | | MDVW | | | | | | F |
| clono | 3077 | GYTF | 3213 | NPNS | 3349 | CARS | 3485 | QGDS | 3621 | GKNN | 3757 | CNSR |
| type297 | | TDH | | GG | | DSGS | | LRSY | | RPS | | DSSG |
| | | | | | | HYVF | | YAS | | | | NHWV |
| | | | | | | FDDW | | | | | | F |
| clono | 3078 | GYTF | 3214 | NPNS | 3350 | CARD | 3486 | QGDS | 3622 | GKNN | 3758 | CNSR |
| type298 | | TGY | | GG | | LDYY | | LRSY | | RPS | | DSSD |
| | | | | | | GSGN | | YAS | | | | NHRV |
| | | | | | | YDYW | | | | | | F |
| clono | 3079 | GFTF | 3215 | NSDG | 3351 | CARN | 3487 | QGDS | 3623 | G?NNRP | 3759 | CNSR |
| type300 | | SSY | | SN | | RDYH | | LRSY | | S | | DS |
| | | | | | | GSGS | | YAS | | | | |
| | | | | | | FDYW | | | | | | SGNH |
| | | | | | | | | | | | | WVF |
| clono | 3080 | GDNV | 3216 | YYRS | 3352 | CARD | 3488 | QGDS | 3624 | GKNN | 3760 | CNSR |
| type301 | | SSNS | | KWY | | WNFA | | LRSY | | RPS | | DSSG |
| | | A | | | | FDIW | | YAS | | | | NHLV |
| | | | | | | | | | | | | F |
| clono | 3081 | GYTF | 3217 | SAYN | 3353 | CART | 3489 | SGSS | 3625 | HDDL | 3761 | CAAW |
| type302 | | TSY | | GN | | IFGV | | SNIG | | LPS | | DARL |
| | | | | | | VNNW | | YNAV | | | | NGWV |
| | | | | | | FDPW | | N | | | | F |
| clono | 3082 | GYTF | 3218 | NPNS | 3354 | CARD | 3490 | TGTS | 3626 | DVSK | 3762 | CCSY |
| type303 | | TGY | | GG | | GEQL | | SDVG | | RPS | | AGSY |
| | | | | | | ALNW | | GYNY | | | | TWVF |
| | | | | | | FDPW | | VS | | | | |
| clono | 3083 | GFTF | 3219 | ISST | 3355 | CARE | 3491 | TGSS | 3627 | DNSD | 3763 | CQSY |
| type304 | | SRY | | SY | | TPVT | | SNIG | | RPS | | DSSL |
| | | | | | | LFDA | | ARYD | | | | SGSV |
| | | | | | | FDIW | | VH | | | | F |
| clono | 3084 | GFTF | 3220 | SWNS | 3356 | CAKD | 3492 | TGSS | 3628 | GNSN | 3764 | CQSY |
| type305 | | DEY | | GS | | IFTG | | SNIG | | RPS | | DSSL |
| | | | | | | RAGY | | AGYD | | | | SGWV |
| | | | | | | FDYW | | VH | | | | F |
| clono | 3085 | GFTF | 3221 | WYDG | 3357 | CARA | 3493 | TGSS | 3629 | GNSN | 3765 | CQSY |
| type306 | | SSY | | SN | | ITGT | | SNIG | | RPS | | DSSL |
| | | | | | | TGNW | | AGYD | | | | SGWV |
| | | | | | | FDPW | | VH | | | | F |
| clono | 3086 | GFSI | 3222 | FWND | 3358 | CTHT | 3494 | TLRS | 3630 | YKSD | 3766 | CMIW |
| type307 | | STSG | | D | | EYGS | | GINV | | SDKH | | HSSA |
| | | V | | | | SWSV | | GTSR | | QDS | | VVF |
| | | | | | | DYW | | IY | | | | |
| clono | 3087 | GFTF | 3223 | NWNG | 3359 | CARH | 3495 | TLRS | 3631 | YKSD | 3767 | CMIW |
| type308 | | DDY | | GS | | FDWL | | GINV | | SDKQ | | HSSA |
| | | | | | | LSNA | | GTYR | | QGS | | SVVF |
| | | | | | | FDIW | | TY | | | | |
| clono | 3088 | GYTF | 3224 | NPNS | 3360 | CVRR | 3496 | SGAK | 3632 | QDRK | 3768 | CQAW |
| type309 | | TSY | | GN | | ITVV | | LGDK | | RPS | | DSST |
| | | | | | | RGVI | | YAC | | | | AVF |
| | | | | | | SLDY | | | | | | |
| | | | | | | W | | | | | | |
| clono | 3089 | GFTF | 3225 | SSSS | 3361 | CARE | 3497 | SGDA | 3633 | EDSK | 3769 | CYST |
| type310 | | SSY | | SY | | TYYY | | LPKK | | RPS | | DSSG |
| | | | | | | DSSG | | YAY | | | | NHRV |
| | | | | | | YFDY | | | | | | F |
| | | | | | | W | | | | | | |
| clono | 3090 | GFTF | 3226 | KQDG | 3362 | CARD | 3498 | QGDS | 3634 | GKNN | 3770 | CNSR |
| type316 | | SSY | | SE | | DTIF | | LRSY | | RPS | | DSSG |
| | | | | | | GVVT | | YAS | | | | NLF |
| | | | | | | DAFD | | | | | | |
| | | | | | | IW | | | | | | |
| clono | 3091 | GDSV | 3227 | YYRS | 3363 | CARG | 3499 | SGDK | 3635 | QDSK | 3771 | CQAW |
| type318 | | SSNS | | KWY | | VGAR | | LGDK | | RPS | | DSST |
| | | A | | | | GWFD | | YAC | | | | AVF |
| | | | | | | PW | | | | | | |
| clono | 3092 | GFTF | 3228 | SSSS | 3364 | CARD | 3500 | QGDS | 3636 | GKNN | 3772 | CNSR |
| type319 | | STY | | TY | | PPLS | | LRSY | | RPS | | DSSG |
| | | | | | | GSYA | | YAS | | | | NHWV |
| | | | | | | GEFD | | | | | | F |
| | | | | | | YW | | | | | | |
| clono | 3093 | GFSL | 3229 | DWDD | 3365 | CARR | 3501 | SGDA | 3637 | EDSK | 3773 | CYST |
| type320 | | STSG | | D | | RGYS | | LPKK | | RPS | | DSSG |
| | | M | | | | YGWG | | YAY | | | | NHRV |
| | | | | | | DFDY | | | | | | F |
| | | | | | | W | | | | | | |
| clono | 3094 | EFIF | 3230 | SISS | 3366 | CARG | 3502 | SGDA | 3638 | EDSK | 3774 | CYST |
| type322 | | RSY | | RT | | GLLN | | LPKK | | RPS | | DSSG |
| | | | | | | WNYE | | YAY | | | | NHRV |
| | | | | | | GWFD | | | | | | F |
| | | | | | | PW | | | | | | |
| clono | 3095 | GFTF | 3231 | SSSG | 3367 | CARD | 3503 | SGDA | 3639 | EDSK | 3775 | CYST |
| type323 | | SDY | | ST | | GGIA | | LPKK | | RPS | | DSSG |
| | | | | | | ARPD | | YAY | | | | NHRV |
| | | | | | | WYFD | | | | | | F |
| | | | | | | LW | | | | | | |
| clono | 3096 | GFTF | 3232 | SSSS | 3368 | CART | 3504 | TGSS | 3640 | GN SNRP | 3776 | CQSY |
| type326 | | SSY | | ST | | YYYG | | SNIG | | S | | DSSL |
| | | | | | | SGSY | | AGYD | | | | SGVV |
| | | | | | | YTLD | | VH | | | | F |
| | | | | | | YW | | | | | | |
| clono | 3097 | GYTF | 3233 | NPNS | 3369 | CARG | 3505 | TLRS | 3641 | YKSD | 3777 | CMIW |
| type327 | | TSY | | GN | | GITI | | GINV | | SDKQ | | HSSA |
| | | | | | | FGVV | | GTYR | | QGS | | WVF |
| | | | | | | TPFD | | IY | | | | |
| | | | | | | YW | | | | | | |
| clono | 3098 | GGSI | 3234 | YYSG | 3370 | CARD | 3506 | SGDK | 3642 | QDSK | 3778 | CQAW |
| type328 | | SSGG | | S | | ALHY | | LGDK | | RPS | | DSST |
| | | Y | | | | YGSG | | YAC | | | | VVF |
| | | | | | | SAFD | | | | | | |
| | | | | | | YW | | | | | | |
| clono | 3099 | GFTF | 3235 | KQDG | 3371 | CARE | 3507 | QGDS | 3643 | GKNN | 3779 | CNSR |
| type333 | | SSY | | SE | | GVLW | | LRRY | | RPS | | DSSG |
| | | | | | | FGEF | | YAS | | | | NHLV |
| | | | | | | YYYM | | | | | | F |
| | | | | | | DVW | | | | | | |
| clono | 3100 | GFTF | 3236 | SSSS | 3372 | CARD | 3508 | SGDA | 3644 | EDSK | 3780 | CYST |
| type339 | | SSY | | ST | | GDYY | | LPKK | | RPS | | DSSG |
| | | | | | | DSSG | | YAY | | | | NHRV |
| | | | | | | YYHF | | | | | | F |
| | | | | | | DYW | | | | | | |
| clono | 3101 | GFTF | 3237 | SGSG | 3373 | CAKD | 3509 | SGDA | 3645 | EDSK | 3781 | CYST |
| type340 | | SSY | | GS | | RGGE | | LPKK | | RPS | | DSSG |
| | | | | | | NWNY | | YAY | | | | NHRV |
| | | | | | | GGWF | | | | | | F |
| | | | | | | DPW | | | | | | |
| clono | 3102 | GFTF | 3238 | WYDG | 3374 | CAGA | 3510 | GGNN | 3646 | YNRD | 3782 | CQVW |
| type341 | | SSY | | SN | | YYYD | | IGGK | | RPS | | DSSS |
| | | | | | | SSGY | | SVH | | | | DHPV |
| | | | | | | LNYM | | | | | | F |
| | | | | | | DVW | | | | | | |
| clono | 3103 | GFTF | 3239 | KTKT | 3375 | CTTD | 3511 | SGSN | 3647 | SNNQ | 3783 | CSSY |
| type342 | | RNA | | DGGA | | HIEY | | SNIG | | RPS | | AGSN |
| | | | | | | SSLY | | FNTV | | | | NFVF |
| | | | | | | YFDY | | N | | | | |
| | | | | | | W | | | | | | |
| clono | 3104 | GYTF | 3240 | SAYN | 3376 | CARQ | 3512 | TGTS | 3648 | EVSK | 3784 | CSSY |
| type343 | | TSY | | GN | | LAYC | | SDVG | | RPS | | AGSN |
| | | | | | | GGDC | | GYNY | | | | NLVF |
| | | | | | | YLYF | | VS | | | | |
| | | | | | | DYW | | | | | | |
| clono | 3105 | GDSV | 3241 | YYRS | 3377 | CARE | 3513 | TGTS | 3649 | EVSK | 3785 | CSSY |
| type345 | | SSNS | | KWY | | AYWN | | SDVG | | RPS | | AGSN |
| | | A | | | | YGGF | | GYNY | | | | NFGV |
| | | | | | | DYW | | VS | | | | F |
| clono | 3106 | GFTF | 3242 | SSSS | 3378 | CARD | 3514 | SGDK | 3650 | QDNK | 3786 | CQAW |
| type349 | | SSY | | SY | | GRIT | | LGNK | | RPS | | DSST |
| | | | | | | MVRG | | YAC | | | | VVF |
| | | | | | | VRNW | | | | | | |
| | | | | | | FDPW | | | | | | |
| clono | 3107 | GFTF | 3243 | SSST | 3379 | CARM | 3515 | SGDK | 3651 | QDIK | 3787 | CQAW |
| type350 | | SSY | | ST | | SSQL | | LGDK | | RPS | | DSST |
| | | | | | | ELHY | | YAC | | | | VVF |
| | | | | | | YCYY | | | | | | |
| | | | | | | MDVW | | | | | | |
| clono | 3108 | GFTF | 3244 | KSKT | 3380 | CTTD | 3516 | SGDK | 3652 | QDSM | 3788 | CQAW |
| type351 | | SNA | | DGGT | | LGYS | | LGDK | | RPS | | DSST |
| | | | | | | GYDW | | YAC | | | | VVF |
| | | | | | | GAFD | | | | | | |
| | | | | | | YW | | | | | | |
| clono | 3109 | GDSV | 3245 | YYRS | 3381 | CARD | 3517 | SGDK | 3653 | QDSK | 3789 | CQAW |
| type352 | | SSNS | | KWY | | RVNW | | LGDK | | RPS | | DSST |
| | | A | | | | NDVG | | YAC | | | | VVF |
| | | | | | | FDYW | | | | | | |
| clono | 3110 | GFTF | 3246 | KQDG | 3382 | CART | 3518 | SGDA | 3654 | EDSK | 3790 | CYST |
| type356 | | SSY | | SE | | PGYS | | LPKK | | RPS | | DSSG |
| | | | | | | SSWY | | YAY | | | | NHVV |
| | | | | | | EGPY | | | | | | F |
| | | | | | | FDYW | | | | | | |
| clono | 3111 | GGSI | 3247 | YYSG | 3383 | CARE | 3519 | SGDA | 3655 | EDSK | 3791 | CYST |
| type358 | | TTRS | | N | | DLIG | | LPKK | | RPS | | DSSG |
| | | Y | | | | NDYW | | YAY | | | | NHRV |
| | | | | | | | | | | | | F |
| clono | 3112 | GGSI | 3248 | YYSG | 3384 | CARE | 3520 | SGSS | 3656 | FNNQ | 3792 | CAAW |
| type359 | | STRS | | S | | DLIG | | SNIG | | RPS | | DDSL |
| | | Y | | | | NDYW | | INTV | | | | KVF |
| | | | | | | | | N | | | | |
| clono | 3113 | GGSF | 3249 | NHSG | 3385 | CARE | 3521 | ASST | 3657 | STSN | 3793 | CLLY |
| type360 | | SGH | | F | | GLTG | | GAVT | | KHS | | YGGA |
| | | | | | | HVFD | | SGYY | | | | QVF |
| | | | | | | IW | | PN | | | | |
| clono | 3114 | GGFL | 3250 | NHSG | 3386 | CARE | 3522 | TGTS | 3658 | DVSK | 3794 | CCSY |
| type361 | | RGY | | S | | GLTG | | SDVG | | RPS | | AGSS |
| | | | | | | HTFD | | GYNY | | | | TVVE |
| | | | | | | IW | | VS | | | | |
| clono | 3115 | GYIF | 3251 | NPYN | 3387 | CARG | 3523 | SGNL | 3659 | TDCE | 3795 | CFSA |
| type363 | | SNY | | VN | | VWGS | | LAKK | | RPS | | ADNT |
| | | | | | | YRSH | | YPR | | | | SVF |
| | | | | | | SYYT | | | | | | |
| | | | | | | FMDV | | | | | | |
| | | | | | | W | | | | | | |
| clono | 3116 | GFTF | 3252 | SSSS | 3388 | CARD | 3524 | QGIS | 3660 | GKNN | 3796 | CNSR |
| type364 | | SSY | | SY | | YRPY | | LRSY | | RPS | | DSSG |
| | | | | | | YDIL | | YAS | | | | NHVV |
| | | | | | | TGYS | | | | | | |
| | | | | | | HFDY | | | | | | |
| | | | | | | W | | | | | | |
| clono | 3117 | GFTF | 3253 | TPSG | 3389 | CARR | 3525 | SGDA | 3661 | EDSK | 3797 | CYST |
| type365 | | TSY | | GT | | VLWF | | LPKK | | RPS | | DSSG |
| | | | | | | GELR | | YAY | | | | NHVV |
| | | | | | | DYFY | | | | | | F |
| | | | | | | YMDV | | | | | | |
| | | | | | | W | | | | | | |
| clono | 3118 | GGSI | 3254 | FYSG | 3390 | CVRQ | 3526 | TGTS | 3662 | EVSK | 3798 | CSSY |
| type366 | | STRS | | S | | GYDS | | SDVG | | RPS | | AGSN |
| | | Y | | | | WTGY | | GYNY | | | | NLVF |
| | | | | | | SFFY | | VS | | | | |
| | | | | | | FDYW | | | | | | |
| clono | 3119 | GGSF | 3255 | NHSG | 3391 | CARG | 3527 | SGSS | 3663 | SSNQ | 3799 | CTSW |
| type367 | | SVY | | S | | GGYS | | SNIG | | RPS | | DDSL |
| | | | | | | FGGF | | SKTV | | | | NTWV |
| | | | | | | DYW | | N | | | | F |
| clono | 3120 | GGSI | 3256 | YYSG | 3392 | CARQ | 3528 | GENN | 3664 | RDSD | 3800 | CQVW |
| type368 | | SSII | | S | | NWGS | | IGSR | | RPS | | DSST |
| | | Y | | | | DAFD | | NVH | | | | AVF |
| | | | | | | IW | | | | | | |
| clono | 3121 | GGSF | 3257 | NHSG | 3393 | CARG | 3529 | QGDS | 3665 | GKNN | 3801 | CNSR |
| type369 | | SGY | | N | | ELGI | | LRSY | | RPS | | DSSG |
| | | | | | | GYWY | | YAS | | | | NHVV |
| | | | | | | FDLW | | | | | | F |
| clono | 3122 | GGSF | 3258 | NHSG | 3394 | CARE | 3530 | SGDA | 3666 | EDSK | 3802 | CYST |
| type370 | | SGY | | S | | GGTT | | LPKK | | RPS | | DSSG |
| | | | | | | HEPL | | YAY | | | | NHRV |
| | | | | | | FDYW | | | | | | F |
| clono | 3123 | GYTF | 3259 | SAYN | 3395 | CARA | 3531 | TGTS | 3667 | DVSK | 3803 | CCSY |
| type379 | | ITY | | GN | | PGGS | | SDVG | | RPS | | AGSS |
| | | | | | | CGST | | GYNY | | | | TLVE |
| | | | | | | NCYK | | VS | | | | |
| | | | | | | WNYD | | | | | | |
| | | | | | | PYYF | | | | | | |
| | | | | | | DYW | | | | | | |
| clono | 3124 | GYTF | 3260 | SSYN | 3396 | CARA | 3532 | TGTS | 3668 | DVSK | 3804 | CCSY |
| type380 | | ITY | | GN | | PGGD | | SDVG | | RPS | | AGSS |
| | | | | | | CSST | | GYNH | | | | TLVF |
| | | | | | | SCYK | | VS | | | | |
| | | | | | | WNYD | | | | | | |
| | | | | | | PYYF | | | | | | |
| | | | | | | DYW | | | | | | |

TABLE 10
Hybridoma Antibody Sequences I

| Hybridoma | SEQ | | SEQ | |
|---|---|---|---|---|
| clone | ID NO | VH Sequence | ID NO | VK Sequence |
| M1 | 1053 | QVQLQESGPGLVKPSQTLSLTC | 1070 | DIQMTQSPSSLSASVGDRVTI |
| | | TVSGGSISSGGYYWSWIRQHPG | | TCRASQGIRIDLGWYQQKPGK |
| | | KGLEWIGNIYYSGNPYYNPSLK | | APKRLIYAASSLKSGVPSRFS |
| | | SRLIISVDTSKNQFSLRLNSVT | | GSGSGTEFTLTISSLQPEDFT |
| | | AADTAVYYCATFYYGSGSYYNE | | TYFCLQHNSYPYTFGQGTKLE |
| | | DYWGQGTLVTVSS | | IK |
| M2 | 1054 | QVQLVESGGGLVKPGGSLRLSC | 1071 | DIQMTQSPSSLSASVGDRVTI |
| | | AASGFTFSDSYMSWIRQAPGKG | | TCRASQGIRNDLGWFQQKPGK |
| | | LEWVSYISNSGSYMYYADSVKG | | APKRLIYAASRLQSGVPSRES |
| | | RFTISRDNAKNSLYLQMNNLRA | | GSGSGTEFTLTISSLQPEDFA |
| | | EDTAVYYCARDKLGIGDYWGQG | | TYCCLQHHTYPPTFGQGTKVE |
| | | TLVTVSS | | IK |
| M3 | 1055 | QVQLQESGPGLVKPSETLSLTC | 1072 | DIVMTQSPSSLSPSVGDRVTI |
| | | TVSGGSISSYDWSWIRQPPGKG | | TCRASQGINNYLAWYQQKPGK |
| | | LEWIGYIYYSGSTNYNPSLKSR | | VPQLLIYAASTLQSGVPSRES |
| | | VTISVDTSKNQFSLKLRSVTAA | | GSGSGTDFTLTISSLQPEDVA |
| | | DTAVYYCARKYSYGPFDNWGQG | | TYYCQKYNSXPFTFGPGTKVD |
| | | TLVTVSS | | IK |
| M9 | 1056 | QVQLQESGPGLVKPSQTLSLTC | 1073 | DIQMTQSPSSLSASVGDRVTI |
| | | DISGDSVSSNSAAWNWIRQSPS | | TCRASQSISSYLNWYQQKPGK |
| | | RGLEWLGRTYYRSKWYNDYAVA | | APKLLIYAASSLQSGVPSRFS |
| | | VKSRITINPDTSKNQFSLQLNS | | GSGSGTDFTLTISSLQPEDFA |
| | | VTPEDTAVYYCARESSGWYEDY | | TYYCQQSYSTPFTFGPGTKVD |
| | | YYYYMDVWGKGTTVTVSS | | IK |
| M19 | 1057 | QLQLQESGPGLVKPSETLSLTC | 1074 | DIVMTQSPSSLSASVGDRVTI |
| | | TVSGGSISSSNHYWGWIRQPPG | | TCRASQGIRDDLGWYQQKPEK |
| | | KGLEWIGTLYYSGSTYYEPSLK | | APKRLICAASSLQSGVPSRES |
| | | SRVTISVDTSMNQFSLNLSSVT | | GSGSGTEFTLTISSLQPEDFA |
| | | ATDTAVYNCARGDRYGPFDYWG | | TYYCLQYNRYPWTFGQGTKVE |
| | | QGTLVTVSS | | IK |
| M24 | 1058 | EVQLVESGGGLVKPGGSLRLSC | 1075 | DIQMTQSPSSLSASIGDRVTI |
| | | AASGFTFTNAWMNWVRQAPGKG | | SCRASQSISSYLNWYQQKPGK |
| | | LEWIGRIKSKTAGETTDYAAPV | | APKLLIYGASSLQSGVPSRES |
| | | KGRFTISRDDSKNTLYLQMNSL | | GSGSGTDFTLTISSLQPEDFA |
| | | KTEDTAVYYCTTDPDYGDPYYY | | TYYCQQSYSLPLTFGGGTKVE |
| | | YYFMDVWGKGTTVTVSS | | IK |
| M26 | 1059 | QVQLVQSGVEVKKPGASVKVSC | 1076 | DIVMTQSPLSLPVTPGEPASI |
| | | KASGYAFSNNDISWVRQAPGQG | | SCRSSQSLLHSNGYNYLDWYL |
| | | LEWMAWITTSNGNTNYAPKLQG | | QKPGQSPQLLIYLGSNRASGV |
| | | RVTMTTDTSTSTAYMELRSLKS | | PDRFSGSGSGTDSTLKISRVE |
| | | DDTAVYYCARGGRTGYFDYWGQ | | AEDVGVYYCMQVLQIPLTFGG |
| | | GTLVTVSS | | GTKVEIR |
| M37 | 1060 | QVQLQESGPGLVKPSGTLSLTC | 1077 | DIQMTQSPSSLSASVGDRVTI |
| | | AVSGGSITTNNWWSWVRQSPGK | | TCRASQSISNYLNWYQQKPGK |
| | | GLEWIGEIYHSGNTNYNPSLKS | | APKLLIYAASSLQSGVPSRES |
| | | RVTMSVDKSKNQFSLNLNSVIV | | GSGSGTDFTLTISSLQPEDFT |
| | | ADTAVYYCASALGTYYGAFDTW | | TYYCQQSYGTPYTFGQGAKLQ |
| | | GQGTMVTVSA | | IK |
| M38 | 1061 | QVQLVESGGGVVQPGRSLRLSC | 1078 | DIQMTQSPSSLSASVGDRVTI |
| | | AASGFTESSYGMHWVRQAPGKG | | TCQASQDIRNYLNWYQQKPGK |
| | | LEWVAFIWYDGRNKNYVDSVKG | | APKLLIYDASNLETGVPSRFS |
| | | RFTISRDNSKNTLYLQMNSLRA | | GSGSGTDFTFTISSLKPEDIA |
| | | EDTAVYYCARDRGDYVFDYWGQ | | TYYCQQYDNLLFTFGPGTKVD |
| | | GTLVTVSS | | IK |
| M41 | 1062 | QVQLVESGGGLVQPGRSLRLSC | 1079 | ETKLTQSPGTLSLSPGERTTL |
| | | AASGFTEDDYAIHWVRQAPGKG | | SCRASQSISNNYLAWYQQKPG |
| | | LEWVSGISYNSENIGYADSVKG | | QAPRLLIYRASTRATGIPDRF |
| | | RFTISRDNAKNSLYLQMNSLRS | | SGSGSGTDFTLTIRRLEPEDF |
| | | EDTALYYCAKDMFLTWFSSFDY | | AVYYCQRYGRSPLTFGGGTKV |
| | | WGQGTLVTVSS | | EIK |
| M43 | 1063 | QVQLQESGPGLVKPSGTLSLTC | 1080 | DIVMTQSPSSLSASVGDRVTI |
| | | AVSGGSISSSNWWSWVRQPPGK | | TCRASQTISSYLNWYQQKPGK |
| | | GLEWIGEIYHSGSINYNPSLKS | | APKLLICAASSLQGGVPSRES |
| | | RVTISVDKSKNQFSLKLTSVTA | | GSGSGTDFTLTISSLQPEDFA |
| | | ADTAVYYCASALGNYYGAFDLW | | PYYCQQSYSTPYTFGQGTKLE |
| | | GQGTMVTVSS | | IK |
| M52 | 1064 | QLQLQESGPGLAKPSETLSLTC | 1081 | DIQMTQSPSSLSASVGDRVTI |
| | | TVSGVSISSNSYYWGWIRQPPG | | TCRASQGIRNDLGWYQQKPGK |
| | | KGLEWIGNIYHSGRTYYNPSLR | | APKRLIYAASSLQSGIPSRES |
| | | SRVTISVDTSKNQFSLKLNSVT | | GSGSGTEFTLTISSLQPEDFA |
| | | AADTAVYYCARGYSYGAFDYWG | | TYYCLQHNNYPWTFGQGTKVE |
| | | QGTLVTVSS | | IK |
| M54 | 1065 | QLQLQESGPGLVKPSETLSLTC | 1082 | EIVMTQSPATLSVSPGERATL |
| | | SVSGGSISSSGYYWGWIRQPPG | | SCRASQSVSSNLAWYQLKPGQ |
| | | KGLDWIGTIYYSGNTNYNPSLN | | APRLLIYGASTRATGIPARFS |
| | | SRVTISVDTSRNQFSLKLRSVT | | GSGSGTEFTLTISSLQSEDFA |
| | | AADTAVYYCARGYSYGPFDYWG | | VYYCHPYNNWPLTFGGGTKVE |
| | | QGTLVTVSS | | IK |
| M71 | 1066 | QVQLVESGAEVKKPRASVKVSC | 1083 | EIVMTQSPATLSVSPGERATL |
| | | KTSGYTFTRHYMHWVRQAPGQG | | SCRASQSVSSNLAWYQLKPGQ |
| | | LEWMGIINPNNNSTSYAQKFQG | | APRLLIYGASTRATGIPARFS |
| | | RITMTRDTSTSTVYMELSSLRS | | GSGSGTEFTLTISSLQSEDFA |
| | | EDTAVYYCARFRIVGTTLYFDY | | VYYCHPYNNWPLTFGGGTKVE |
| | | WGQGTLVTVSS | | IK |
| M80 | 1067 | QVQLVESGGGLVKPGGSLRLSC | 1084 | DIVMTQSPLSLPVTPGEPASI |
| | | AASGFTFSDSYMSWIRQAPGKG | | SCRSSQSLLHSNGYNYLDWYL |
| | | LEWVSYISNSGSYMYYADSVKG | | QKPGQSPQLLIYLGSNRASGV |
| | | RFTISRDNAKNSLYLQMNNLRA | | PDRFSGSGSGTDSTLKISRVE |
| | | EDTAVYYCARDKLGIGDYWGQG | | AEDVGVYYCMQVLQIPLTFGG |
| | | TLVTVSS | | GTKVEIR |
| M82 | 1068 | QVQLVQSGAEVKKPGASVKVSC | 1085 | EIQLTQSPGTLSLSPGERATL |
| | | KASGYTFSSFGITWIRQAPGQG | | SCRASQSVRNSYLAWYQQKPG |
| | | LEWMGWISGYTGNTNYAQNLQG | | QAPRLLTYGASSRATGIPDRF |
| | | RVTITTDTSTNTAYMELRSLKS | | SGSGSGTDFTLTISRLEPEDC |
| | | DDTAVYYCAREPVLNPNYYYFY | | AVYFCQQYGSSPTFGGGTKVD |
| | | YMDVWGQGTTVTVSS | | IK |
| M87 | 1069 | QVQLVQSGAEVKKPGASMKVSC | 1086 | DIVMTQSPDSLAVSLGERATI |
| | | KASGYTFTQNHISWVRQAPGQG | | NCKSSQSVLYSSNNKNYLAWY |
| | | LEWMGWISAYSGNTNYAWKFQG | | QQKPGQPPKLLIYWASTRESG |
| | | RVTMTTDTSTNTAYMELRSLRS | | VPDRESGSGSGTDFTLTISSL |
| | | DDTAVYYCARDRGNWNDFAYWG | | QAEDVAVYYCQQYYSTPYTFG |
| | | RGTLVTVSS | | QGTKLEIK |

TABLE 11
Hybridoma Antibody Sequences II

| | SEQ | | SEQ | |
|---|---|---|---|---|
| Hybridoma | ID | | ID | |
| clone | NO | VH Sequence | NO | VK Sequence |
| N14 | 3805 | QVQLVESGAEVKKPRASVKVSCKTSGY | 3810 | DIQLTQSPSFLSASVGDRVTITCRA |
| | | TFTRHYMHWVRQAPGQGLEWMGIINPN | | SQGISSYLAWYQQKPGKAPKLLIYA |
| | | NNSTSYAQKFQGRITMTRDTSTSTVYM | | ASTLQSGVPSRFSGSGSGTEFTLTI |
| | | ELSSLRSEDTAVYYCARFRIVGTTLYF | | SSLQPEDFATYYCQQLNSYPFTFGP |
| | | DYWGQGTLVTVSS | | GTKVDIK |
| N38 | 3806 | QVQLVESGGGLVKPGGSLRLSCAASGF | 3811 | DIVMTQSPLFLPATPGEPASISCRS |
| | | TFSDYYMTWIRQAPGKGLEWVSYIVSS | | TQSLLDSNGYKYLDWYLQKPGQSPQ |
| | | GNIMYYADSVKGRFTISRDNAKNSLYL | | LLIYLGSNRASGVPDRESGSGSGID |
| | | QMNSLRAEDTAVYYCARGGPTGYFDYW | | FTLKISRVEPADTGLYYCMQTRQTP |
| | | GQGALVTVSS | | LTFGRGTRLEIK |
| N68 | 3807 | QVQLVQSGAEVKKPGASVKVSCKASGY | 3812 | DIRLTQSPSSLSASVGDRVTITCRA |
| | | TFTSYGISWVRQAPGQGLEWMGWISAY | | SQSISSHLNWYQQKPGKAPKLLIYA |
| | | NGNTNYAQKLQGRVTMTTDTSTSTAYM | | ASSLQSGVPSRFSGSGSGTDFTLTI |
| | | ELRSLRSDDTAVYYCASHSGSYPNWFD | | SSLQPEDFATYYCQQSYSTPYTFGQ |
| | | PWGQGTLVTVSS | | GTKLEIK |
| N135 | 3808 | QVQLVQSGAEVKKPGASVKVSCKASGY | 3813 | EIVLTQSPATLSLSPGERATLSCRA |
| | | TFTDYYLHWVRQAPGLGLEWMGWINPN | | SQSIWKYLGWYQQKPGQAPRLIISD |
| | | SGVTNFAQKFQDRVTMTRDTSISTAYM | | ASNRATGIPARFSGSGSGTDETLTI |
| | | ELNRLRSDDAAIYYCAKDMAPAVGPEW | | SSLEPEDFAVYFCQHRSNWPLTFGG |
| | | YFDLWGRGTLVTVSS | | GTKVEIK |
| N202 | 3809 | QVQLQESGPGLVKPSETLSLTCTVSGG | 3814 | DIQLTQSPSFLSASVGDRVTITCRA |
| | | SISSYYWSWIRQPPGKGLEWIGYIYYS | | SQGISSYLAWYQQKPGKAPKLLIYA |
| | | GSTNYNPSLKSRVTISVDTSKNQFSLK | | ASTLQSGVPSRFSGSGSGTEFTLTI |
| | | LSSVTAADTAVYYCARDNNWNDGAFDI | | SSLQPEDFATYYCQQLNSYPFTFGP |
| | | WGQGTMVTVSS | | GTKVDIK |

TABLE 12
VH-CDR3 and VL-CDR3 Sequences for Hybridoma Clones in Table 11

| Hybridoma clone | SEQ ID NO | HCDR3 Sequence | SEQ ID NO | LCDR3 Sequence |
|---|---|---|---|---|
| N14 | 3815 | CARFRIVGTTLYFDYW | 3820 | CQQLNSYPFTF |
| N38 | 3816 | CARGGPTGYFDYW | 3821 | CMQTRQTPLTF |
| N68 | 3817 | CASHSGSYPNWFDPW | 3822 | CQQSYSTPYTF |
| N135 | 3818 | CAKDMAPAVGPEWYFDLW | 3823 | CQHRSNWPLTF |
| N202 | 3819 | CARDNNWNDGAFDIW | 3820 | CQQLNSYPFTF |

TABLE 13
Full Heavy Chain (HC) and Light Chain (LC) Sequences for EPOR/CD131 Binders II

| | SEQ | | SEQ | |
|---|---|---|---|---|
| clonotype_ | ID | | ID | |
| id | NO | Full HC AA Sequence | NO | Full LC AA Sequence |
| clonotype | 3824 | QVQLVQSGAEVKKPGASVKVSCKT | 3922 | DIQMTQSPSSLSASVGDRVTITC |
| 6-1 | | SGYTFTSYGISWVRQAPGQGLEWM | | RASQSISSYLNWYQQKPGKAPKL |
| | | GWISAYNGNTNYAQKVQGRVTMIT | | LIYAASSLQSGVPSRFSGSGSGT |
| | | DTSTSTAYMELRSLRSDDTAVYYC | | DFTLTISSLQPEDFATYYCQQSY |
| | | ARAGPGIVAAGSFDYWGQGTLVTV | | STPLTFGGGTKVEIK |
| | | SS | | |
| clonotype | 3825 | EVQLVESGGGLVQPGGSLRLSCTA | 3923 | SYELTQPPSVSVSPGQTARITCS |
| 22-1 | | SGITFSSFWMSWVRQAPGKGLEWV | | GDALPKKYAYWYQQKSGQAPVLV |
| | | ANINQDGSAKYFVDSVKGRFTISR | | IYEDSKRPSGIPERFSGSSSGTM |
| | | DNAKNSLYLQMNSLRAEDTAVYYC | | ATLTISGAQVEDEADYYCYSTDS |
| | | ARERGAARHYYYYYMDVWGTGTTV | | SGNHYVFGTGTKVTVL |
| | | TVSS | | |
| clonotype | 3826 | EVQLVESGGGLVQPGGSLRLSCAA | 3924 | EIVMTQSPATLSVSPGERATLSC |
| 33-2 | | SGFTFSSYWMSWVRQAPGKGLEWV | | RASQSVSSNLAWYQQKPGQAPRL |
| | | ANIKQDGSEKYYVDSVKGRFTISR | | LIYGASTRATGIPARFSGSGSGT |
| | | DNAKNSLYLQMNSLRAEDTAVYYC | | EFTLTISSLQSEDFAVYYCQQYN |
| | | ARGPGVYYYYYYMDVWGKGTTVTV | | NWPITFGQGTRLEIK |
| | | SS | | |
| clonotype | 3827 | QVHLQQWGAGLLKPSETLSLTCAV | 3925 | QTVVTQEPSLTVSPGGTVTLTCA |
| 41-2 | | YGGSLSGYSWSWIRQTPGKGLEWI | | SSTGAVTSGYYPNWFQQKPGQAP |
| | | GEINHGEYTNYNPSLKSRVTMSVD | | RALIYSTSNKHSWTPARFSGSLL |
| | | TSKNQFSLILSSVTAADTAVYHCV | | GGKAALTLSGVQPEDEAEYYCLL |
| | | REELDHWGQGTLVTVSS | | YYGGAWVFGGGTKLTVL |
| clonotype | 3828 | EVQLVESGGGLVQPGGSLRLSCAA | 3926 | DIQMTQSPSSLSASVGDRVTITC |
| 44-2 | | SGFTFSSYSMNWVRQAPGKGLEWV | | QASQDISNYLNWYQQKPGKAPKL |
| | | SYISSSSSTIYYADSVKGRFTISR | | LIYDASNLETGVPSRFSGSGSGT |
| | | DNAKNSLYLQMNGLRDEDTAVYYC | | DFTFTISSLQPEDIATYYCQQYD |
| | | ARKRYRSNLDYWGQGTLVTVSS | | NLPFTFGPGTKVDIK |
| clonotype | 3829 | EVQLVESGGGLIQPGGSLRLSCAA | 3927 | QSALTQPASVSGSPGQSITISCT |
| 49-2 | | SGFTVSSNYMSWVRQAPGKGLEWV | | GTSSDVGGYNYVSWYQQHPGKAP |
| | | SVIYSGGSTYYADSVKGRFTISRD | | KLMIYDVSKRPSGVSNRFSGSKS |
| | | NSKNTLYLQMNSLRAEDTAVYYCA | | GNTASLTISGLQAEDEADYYCCS |
| | | RARIAAPSFDYWGQGTLVTVSS | | YAGSSTLVFGGGTKLTVL |
| clonotype | 3830 | QVQLVQSGAEVKKPGASVKVSCKA | 3928 | QSVLTQPPSVSGAPGQRVTISCT |
| 52-2 | | SGYTFTSYGISWVRQAPGQGLEWM | | GSSSNIGAGYDVHWYQQLPGTAP |
| | | GWISAYNGNTNYAQKLQGRVTMTT | | KLLIYGNSNRPSGVPDRFSGSKS |
| | | DTSTSTAYMELRSLRSDDTAVYYC | | GTSASLAITGLQAEDEADYYCQS |
| | | ARRAARCFDYWGQGTLVTVSS | | YDSSLSGVVFGGGTKLTVL |
| clonotype | 3831 | QVQLVQSGAEVKKPGASVKVSCKA | 3929 | QSVLTQPPSVSGAPGQRVTISCT |
| 54-2 | | SGYTFTSYDINWVRQATGQGLEWM | | GSSSNIGAGYDVHWYQQLPGTAP |
| | | GWMNPNSGNTGYAQKFQGRVTMTR | | KLLIYGNSNRPSGVPDRFSGSKS |
| | | NTSISTAYMELSSLRSEDTAVYYC | | GTSASLAITGLQAEDEADYYCQS |
| | | ARGRDYWGQGTLVTVSS | | YDSSLSGWVFGGGTKLTVL |
| clonotype | 3832 | EVQLVESGGGLVQPGGSLRLSCAA | 3930 | DVVMTQSPLSLPVTLGQPASISC |
| 56-2 | | SGFTFSSYDMHWVRQATGKGLEWV | | RSSQSLVYSDGNTYLNWFQQRPG |
| | | SAIGTAGDTYYPGSVKGRFTISRE | | QSPRRLIYKVSNRDSGVPDRFSG |
| | | NAKNSLYLQMNSLRAGDTAVYYCA | | SGSGTDFTLKISRVEAEDVGVYY |
| | | RAFDIWGQGTMVTVSS | | CMQGTHWPLTFGGGTKVEIK |
| clonotype | 3833 | QEQLVESGGGVVQPGRSLRLSCAA | 3931 | DVVMTQSPLSLPVTLGQPASISC |
| 57-2 | | SGFTFSSYGMHWVRQAPGKGLEWV | | RSSQSLVYSDGNTYLNWFQQRPG |
| | | AVIWYDGSNKYYADSVKGRFTISR | | QSPRRLIYKVSNRDSGVPDRFSG |
| | | DNSKNTLYLQMNSLRAEDTAVYYC | | SGSGTDFTLKISRVEAEDVGVYY |
| | | AREARSSGRGFDYWGQGTLVTVSS | | CMQGTHWPRTFGQGTKVEIK |
| clonotype | 3834 | EVQLVESGGGLVQPGGSLRLSCAA | 3932 | EIVLTQSPATLSLSPGERATLSC |
| 61-2 | | SGFTFSNYAMSWVRQAPGKGLEWV | | RASQSVSSYLAWYQQKPGQAPRL |
| | | SGISGSGVSTYYADSVKGRFTISR | | LIYDASNRATGIPARFSGSGSGT |
| | | DNSKNTLYLQMNSLRAEDTAVYYC | | DFTLTISSLEPEDFAVYYCQQRS |
| | | AKDKWELLRGFDYWGQGTLVTVSS | | NWYTFGQGTKLEIK |
| clonotype | 3835 | QLQLQESGPGLVKPSETLSLTCTV | 3933 | SYELTQPPSVSVSPGQTARITCS |
| 63-1 | | SGGSISSSSYYWGWIRQPPGKGLE | | GDALPKKYAYWYQQKSGQAPVLV |
| | | WIGSIYYSGSTYYNPSLKSRVTIS | | IYEDSKRPSGIPERFSGSSSGTM |
| | | VDTSKNQFSLKLSSVTAADTAVYY | | ATLTISGAQVEDEADYYCYSTDS |
| | | CARHKAAAGSYYFDYWGQGTPVTV | | SGNQVFGGGTKLTVL |
| | | SS | | |
| clonotype | 3836 | EVQLVESGGGLVQPGGSLRLSCAA | 3934 | EIVLTQSPDFQSVTPKEKVTITC |
| 65-1 | | SGFTFSSYDMHWVRQATGKGLEWV | | RASQSIGSSLHWYQQKPDQSPKL |
| | | SAIGIAGDTYYPGSVKGRFTISRE | | LIKYASQSFSGVPSRFSGSGSGT |
| | | NAKNSLYLQMNSLRAGDTTVYYCA | | DFTLTINSLEAEDAATYYCHQSS |
| | | RDRGRLGGVFDLWGRGTLVTVSS | | SLPYTFGQGTKLEIK |
| clonotype | 3837 | EVQLVESGGGLVQPGGSLRLSCAA | 3935 | DIQMTQSPSSLSASVGDRVTITC |
| 75-2 | | SGFTFSSYSMNWVRQAPGKGLEWV | | RASQGISNYLAWFQQKPGKAPKS |
| | | SYISSSSSTIYYADSVKGRITISR | | LIYAASSLQSGVPSKFSGSGSGT |
| | | DNAKNSLYLQMNSLRDEDTAVYYC | | DFTLTISSLQPEDFATYYCQQYN |
| | | ARKGYSSRWDYWGQGTLVTVSS | | SYPITFGQGTRLEIK |
| clonotype | 3838 | QVHLQQWGAGLLKPSETLSLTCAV | 3936 | QAVVTQEPSLTVSPGGTVTLTCG |
| 78-1 | | YGGSLSGYSWSWIRQTPGKGLEWI | | SSTGAVTSGHYPYWFQQKPGQAP |
| | | GEINHGEYTNYNPSLKSRVTMSVD | | RTLIYDTSNKHSWTPARFSGSLL |
| | | TSKNQFSLILSSVTAADTAVYHCV | | GGKAALTLSGAQPEDEAEYYCLL |
| | | REELDHWGQGTLVTVSS | | SYSGAWVFGGGTKLTVL |
| clonotype | 3839 | EVQLVESGGGLVKPGGSLRLSCAA | 3937 | DIQMTQSPSSVSASVGDRVTITC |
| 80-2 | | SGFTFSSYSMNWVRQAPGKGLEWV | | RASQGISSWLAWYQQKPGKAPKL |
| | | SSISSSSSYIYYADSVKGRFTISR | | LIYAASSLQSGVPSRFSGSGSGT |
| | | DNAKNSLYLQMNSLRAEDTAVYYC | | DFTLTISSLQPEDFATYYCQQAN |
| | | AREGNWGFDYWGQGTLVTVSS | | SFPYTFGQGTKLEIK |
| clonotype | 3840 | QVQLVQSGAEVKKPGASVKVSCKA | 3938 | DIQMTQSPSSVSASVGDRVTITC |
| 85-1 | | SGYTFTSYGISWVRQAPGQGLEWM | | RASQGISSWLAWYQQKPGKAPKL |
| | | GWINAYNGNRNYAQKLQGRVTMTT | | LIYAASSLQSGVPSRFSGSGSGT |
| | | DTSTSTAYMELRSLRSDDTAVYCC | | DFTLTISSLQPEDFATYYCQQAN |
| | | ARSHNWNYFYFDYWGQGTLVTVSS | | SFPLTFGGGTKVEIK |
| clonotype | 3841 | QVQLQQSGPGLVKPSQTLSLTCAI | 3939 | QSALTQPPSASGSPGQSVTISCT |
| 87-1 | | SGDSVSSNSAAWNWIRQSPSRGLE | | GTSSDVGGYNYVSWYQQHPGKAP |
| | | WLGRTYYRSKWYNDYAVSVKSRIT | | KLMIYEVSKRPSGVPDRFSGSKS |
| | | INPDTSKNQFSLQLNSVTPEDTAV | | GNTASLTVSGLQAEDEADYYCSS |
| | | YYCARAPVPGWFDPWGQGTLVTVS | | YAGSNNYVFGTGTKVTVL |
| | | S | | |
| clonotype | 3842 | EVQLVESGGGLVKPGGSLRLSCAA | 3940 | SYELTQPPSVSVSPGQTARITCS |
| 91-2 | | SGFTFSSYSMNWVRQAPGKGLEWV | | GDALPKKYAYWYQQKSGQAPVLV |
| | | SSISSSSSYIYYADSVKGRFTISR | | IYEDSKRPSGIPERFSGSSSGTM |
| | | DNAKNSLYLQMNSLRAEDTAVYYC | | ATLTISGAQVEDEADYYCYSTDS |
| | | ARGMGAVAEDWEDPWGQGTLVTVS | | SGNHRVFGGGTKLTVL |
| | | S | | |
| clonotype | 3843 | EVQLVESGGGLVQPGGSLRLSCAA | 3941 | SSELTQDPAVSVALGQTVRITCQ |
| 236-1 | | SGFTFSSYWIHWVRQVPGKGLVWV | | GDSLRSYYASWYQQKPGQAPVLV |
| | | SRINSDGITTSYADSVKGRFTISR | | IYGKNNRPSGIPDRFSGSSSGNT |
| | | DNAKNTLYLQMNSLRAEDTAMYYC | | ASLTITGAQAEDEADYYCNSRDS |
| | | ASFITGTWGQGTLVTVSS | | SGNHWVFGGGTKLTVL |
| clonotype | 3844 | EVQLVESGGGLVQPGRSLRLSCEA | 3942 | DIQMTQSPSSLSASVGDRVTITC |
| 237-1 | | SGFTFDDFAMHWVRQAPGKGLEWV | | RASQGVRNDLGWYQQKPGKVPKR |
| | | SGISWNSGTIGYADSVKGRESISR | | LLYAASRLQSGVPSRFSGTGSGT |
| | | DNAKKSLYLQMTSLRAEDTALYYC | | EFTLTISSLQPEDFATYYCLQHN |
| | | AKLGFDYWGQGTLVTVSS | | SYPYTFGQGTKLEIR |
| clonotype | 3845 | DEQLVESGGGLVQPGGSLRLSCAA | 3943 | QSALTQPASVSGSPGQSITISCT |
| 243-1 | | SGFTFSSFAMSWVRQAPGKGLEWV | | GTSSDVGGYNYVSWYQQHPGKAP |
| | | SAIRGTGVSTYYADSVKGRFTISR | | KLMIYEVITRPSGVSNRFSGSKS |
| | | DNSKNTLYLQMSSLRAEDTAVYYC | | GTTASLTISGLQAEDEADYYCTS |
| | | AKNLFFDYWGQGTLVTVSS | | YTSTNSVVFGGGTRLTVL |
| clonotype | 3846 | QVQLVESGGGVVQPGRSLRLSCAA | 3944 | SYELTQPPSVSVSPGQTARITCS |
| 246-1 | | SGFTFRNSGMHWVRQAPGKGLEWV | | GDALPKKYAYWYQQKSGQAPVLV |
| | | AVIWYDGSNKYYVDSVKGRFTISR | | IYEDSKRPSGIPERFSGSSSGTM |
| | | DNSKNTLYLQMNSLRAEDTAVYYC | | ATLTISGAQVEDEADYYCYSTDS |
| | | ARDNWGIDYWGQGTLVTVSS | | SGNHRVFGGGTKLTVL |
| clonotype | 3847 | EVQLVESGGGLVKPGGSLRLSCAA | 3945 | QSALTQPPSASGSPGQSVTISCT |
| 247-1 | | SGFTFSSYSMNWVRQAPGKGLEWV | | GTSSDVGGYNYVSWYQQHPGKAP |
| | | SSISSSSSYIYYADSVKGRFTISR | | KLMIYEVSKRPSGVPDRFSGSKS |
| | | DNAKNSLYLQMNSLRAEDTAVYYC | | GNTASLTVSGLQAEDEADYYCSS |
| | | AREVTSFDYWGQGTLVTVSS | | YAGSNNLVFGGGTKLTVL |
| clonotype | 3848 | EVQLVESGGGLIQPGGSLRLSCAA | 3946 | SYELTQPLSVSVALGQTARITCG |
| 249-1 | | SGLIVSSNYMSWVRQAPGRGLEWV | | GNNIGSKNVHWYQQKPGQAPVLV |
| | | SVLYSGDGTSYADSVKGRFTISRD | | IYRDSNRPSGIPERFSGSNSGNT |
| | | NSKNTLYLQMNSLRAEDTAVYYCA | | ATLTISRAQAGDEADYYCQVWDS |
| | | RDNWSSYFDYWGQGTLVTVSS | | STWVFGGGTKLTVL |
| clonotype | 3849 | EVQLVESGGGLIQPGGSLRLSCAA | 3947 | SSELTQDPAVSVALGQTVRITCQ |
| 250-1 | | SGFTVSSNYMSWVRQAPGKGLEWV | | GDSLRSYYASWYQQKPGQAPVLV |
| | | SVIYSGGTTYYADSVKGRFTISRD | | IYGKNNRPSGIPDRFSGSSSGNT |
| | | SSKNTLYLQMNSLRAEDTAVYYCA | | ASLTITGAQAEDEADYYCNSRDS |
| | | RGIAARAFAYWGQGTLVTVSS | | SGNHLVFGGGTKLTVL |
| clonotype | 3850 | QVQLVQSGAEVKKPGASVKVSCKA | 3948 | SSELTQDPAVSVALGQTVRITCQ |
| 250-2 | | SGYTFTSYGISWVRQAPGQGLEWM | | GDSLRSYYASWYQQKPGQAPVLV |
| | | GWINTYNNNTNCAQKLQGRVTMTT | | IYGKNNRPSGIPDRFSGSSSGNT |
| | | DTSTSTAYMELRSLRSDDTAVYYC | | ASLTITGAQAEDEADYYCNSRDS |
| | | AREGYLDYWGQGTLVTVSS | | SGNHRVFGGGTKLTVL |
| clonotype | 3851 | QVQLVESGGGLVKPGGSLRLSCAA | 3949 | SYELTQPPSVSVSPGQTASITCS |
| 251-1 | | SGFTFSDYYMSWIRQAPGKGLEWV | | GDKLGDKYACWYQQKPGQSPVLV |
| | | SYISGPGNTIYYADSVKGRFTISR | | IYQDSKRPSGIPERFSGSNSGNT |
| | | DSAINSLYLQMNSLRAEDTALYYC | | ATLTISGTQAMDEADYYCQAWDS |
| | | ARERNWGGFDYWGQGTLVTVSS | | STWVFGGGTKLTVL |
| clonotype | 3852 | EVQLVESGGGLVQPGGSLRLSCAA | 3950 | SYELTQPPSVSVSPGQTARITCS |
| 252-1 | | SGFTFSAYSMNWVRQAPGKGLEWV | | GDALPKKYAYWYQQKSGQAPVLV |
| | | SYINYRNSATYYADSVKGRFSISR | | IYEDSKRPSGIPERFSGSSSGTM |
| | | DTAKNSLYLQMNSLRDEDTAVYYC | | ATLTISGAQVEDEADYYCYSTDS |
| | | ARDYYFYYMDVWGKGTTVTVSS | | SGNHRVFGGGTKLTVL |
| clonotype | 3853 | EVQLVESGGGLVQPGGSLRLSCAA | 3951 | SYELTQPPSVSVSPGQTARITCS |
| 252-2 | | SGFTFSSYSMNWVRQAPGKGLEWV | | GDALPKKYAYWYQQKSGQAPVLV |
| | | SYISSSSSTIYYADSVKGRFTISR | | IYEDSKRPSGIPERFSGSSSGTM |
| | | DNAKNSLYLQMNSLRDEDTAVYYC | | ATLTISGAQVEDEADYYCYSTDS |
| | | ARDSSSWYFDYWGQGTLVTVSS | | SGNHRVFGGGTKLTVL |
| clonotype | 3854 | EVQLVESGGGLVKPGGSLRLSCAA | 3952 | SSELTQDPAVSVALGQTVRITCQ |
| 253-1 | | SGFTFSNAWMSWVRQAPGKGLEWV | | GDSLRSYYASWYQQKPGQAPVLV |
| | | GRIKSKTDGGTTDYAAPVKGRFTI | | IYGKNNRPSGIPDRFSGSSSGNT |
| | | SRDDSKNTLYLQMNSLKTEDTAVY | | ASLTITGAQAEDEADYYCNSRDS |
| | | YCTTVNWAFDYWGQGTLVTVSS | | SGNHWVFGGGTKLTVL |
| clonotype | 3855 | QVQLVESGGGLVKPGGSLRLSCAA | 3953 | QSVLTQPPSASGTPGQRVTISCS |
| 253-2 | | SGFIFSTYYMSWVRQAPGKGLEWV | | GGSSNIGRNPVYWIQQLPGTAPK |
| | | SYISTSGRNIYYADSMNGRFTMSR | | LLIYNNDQRPSGVPDRESGSKSG |
| | | DNAKNSLYLEMNSLRAEDTAVYYC | | TSASLAISGLQSEDEADYYCAAW |
| | | VRDNWNYGFDHWGQGTLVTVSS | | DGRLKVFGGGTKLTVL |
| clonotype | 3856 | QVQLVESGGGVVQPGRSLRLSCAA | 3954 | SSELTQDPAVSVALGQTVRITCQ |
| 254-1 | | SGFTFSSYDMHWVRQAPGKGLEWV | | GDSLRSYYASWYQQKPGQAPVLV |
| | | AVIWYDGSNKYYADSVKGRFTISR | | IYGKNNRPSGIPDRFSGSSSGNT |
| | | DNSKNTLYLQMISLRAEDTAVYYC | | ASLTITGAQAEDEADYYCNSRDS |
| | | ARGIAAAGLDYWGQGTLVTVSS | | SGNHVVFGGGTKLTVL |
| clonotype | 3857 | EVQLMESGGGLVQPGGSLRLSCAA | 3955 | SYELTQPPSVSVSPGQTARITCS |
| 254-2 | | SGFTFSSYWMHWVRQAPGKGLVWV | | GDALPKKYAYWYQQKSGQAPVLV |
| | | SRINSDGSSTSYADSVKGRFAISR | | IYEDSKRPSGIPERFSGSSSGTM |
| | | DNAKNTLFLQMNSLRAEDTAVYYC | | ATLTISGAQVEDEADYYCYSTDS |
| | | AREDSRNWFDPWGQGTLVTVSS | | SGNHRVFGGGTKLTVL |
| clonotype | 3858 | EVQLVESGGGLVQPGGSLKLSCAA | 3956 | SYELTQPPSVSVSPGQTARITCS |
| 255-1 | | SGFTFSGSAMHWVRQASGKGLEWV | | GDALPKKYAYWYQQKSGQAPVLV |
| | | GRIRSKANSYATAYAASVKGRFTI | | IYEDSKRPSGIPERFSGSSSGTM |
| | | SRDDSKNTAYLQMNSLKTEDTAVY | | ATLTISGAQVEDEADYYCYSTDS |
| | | YCTSTGTTEDYWGQGTLVTVSS | | SGNHWVFGGGTKLTVL |
| clonotype | 3859 | EVQLVESGGGLLQPGGSLRLSCAA | 3957 | QAVVTQEPSLTVSPGGTVTLTCG |
| 255-2 | | SGFTFSSYSMNWVRQAPGKGLEWV | | SSTGAVTSGHYPYWFQQKPGQAP |
| | | SYISSGSSTIYYADSVKGRFTISR | | RTLIYDTSNKHSWTPARFSGSLL |
| | | DNAKNSLFLQMNSLRDEDTAVYYC | | GGKAALTLSGAQPEDEAEYYCLL |
| | | ARTSRVAAFDYWGQGTLVTVSS | | SYSGARVFGGGTKLTVL |
| clonotype | 3860 | QVQLVESGGGLVKPGGSLRLSCAA | 3958 | DIQMTQSPSSLSASVGDRVTITC |
| 256-1 | | SGFTFSDYYMSWIRQAPGKGLEWI | | RASQGIGNDLGWYQQKAGKAPKR |
| | | SYISRRGDIISFADSVQGRFTISR | | LIYAASNLQSGVPSRFSGSGSGT |
| | | DNAKNSLYLQMNSLRAEDTAVYYC | | EFTLTISSLQPEDFATYYCLHHN |
| | | ARDDLWITGDYWGQGTPVTVSS | | THPLTFGGGTKVEIK |
| clonotype | 3861 | EVQLVESGGGLVQPGGSLRLSCAA | 3959 | QSALTQPPSASGSPGQSVTISCT |
| 256-2 | | SGFTFSTYWMHWVRQAPGKGLVWV | | GTSSDVGGYNYVSWYQQHPGKAP |
| | | SRINGDGSSTNYADSVKGRFTISR | | KLMIYEVSKRPSGVPDRFSGSKS |
| | | DNAKNTLYLQMNSLRAEDTAVYYC | | GNTASLTVSGLQAEDEADYYCSS |
| | | AREEQRNWFDPWGQGTLVTVSS | | YAGSNNLVFGTGTKVTVL |
| clonotype | 3862 | EVQLVESGGGLVKPGGSLRLSCAA | 3960 | SYELTQPPSVSVSPGQTASITCS |
| 257-1 | | SGFTFSHSGMNWVRRAPGKGLEWV | | GDKLGDKYACWYQQKPGQSPVLV |
| | | SSISSSSSYIYYADSVKGRFTISR | | IYQDSKRPSGIPERFSGSNSGNT |
| | | DNAKNSLYLQMNSLRAEDTAVYYC | | ATLTISGTQAMDEADYYCQAWDS |
| | | ARAYNWNGYFDYWGQGTLVTVSS | | STVVFGGGTKLTVL |
| clonotype | 3863 | EVQLVESGGGLIQPGGSLRLACAA | 3961 | DIQLTQSPSFLSASVGDRVTITC |
| 257-2 | | SGFTVSGNYMNWVRQAPGKGLEWV | | RASQGISSYLAWYQQKPGKAPNL |
| | | SVIYSGGGTYYADSVKGRFTFSSD | | LIYAASTLQSGVPSRFSGSGSGT |
| | | NSKNTVFLQMNSLRAEDTALYYCA | | EFTLTISSLQPEDFAIYYCQQLD |
| | | RDSGDSYAFDLWGQGTMVTVSS | | SYPLTFGGGTKVDIK |
| clonotype | 3864 | QVQLQESGPGLVKPSETLSLTCTV | 3962 | EIVLTQSPATLSLSPGERATLSC |
| 258-1 | | SGGSISSYYWSWIRQPPGKGLEWI | | RASQSVSSYLAWYQQKPGQAPRL |
| | | GYIYYSGSTNYNPSLKSRVTISVD | | LIYDASNRATGIPARFSGSGSGT |
| | | TSKNQFSLKLSSVTAADTAVYYCA | | DETLTISSLEPEDFAVYYCQQRS |
| | | REGGDYYYYMDVWGKGTTVTVSS | | NWPPTFGGGTKVEIK |
| clonotype | 3865 | EVQLVESGGGLVQPGGSLRLSCAA | 3963 | SYELTQPPSVSVSPGQTARITCS |
| 259-1 | | SGFTFSSYSMNWVRQAPGKGLEWV | | GDALPKKYAYWYQQKSGQAPVLV |
| | | SYISSSSSTIYYADSVKGRFTISR | | IYEDSKRPSGIPERFSGSSSGTM |
| | | DNAKNSLYLQMNSLRDEDTAVYYC | | ATLTISGAQVEDEADYYCYSTDS |
| | | ARHDILTGYFDYWGQGTLVTVSS | | SGNHRVFGGGTKLTVL |
| clonotype | 3866 | QVQLVQSGAEVKKPGASVKVSCKA | 3964 | SYELTQPPSVSVSPGQTARITCS |
| 259-2 | | SGYTFTNYGINWVRQAPGQGLEWM | | GDALPKKYAYWYQQKSGQAPVLV |
| | | GWISAYSGNTNYARKLQGRVTMTT | | IYEDSKRPSGIPERFSGSSSGTM |
| | | DTSTITAYMELRSLTSDDTAVYYC | | ATLTISGAQVEDEADYYCYSTDS |
| | | AWYSRDWSDFDYWGQGTLVTVSS | | SGNHRVFGGGTKLTVL |
| clonotype | 3867 | QVQLQESGPGLVKPSGTLSLTCAV | 3965 | EIVLTQSPATLSLSPGERATLSC |
| 260-1 | | SGGSISSNNWWSWVRQPPGKGLEW | | RASQNVNSYLAWYQQKPGQAPRL |
| | | IGEIYHSGSTNYNPSLKSRVTISV | | LIYDASNRATGIPARFSGSGSGT |
| | | DKSKNQFSLKLSSVTAADTAVYYC | | DFTLTISSLEPEDFAVYYCQQRS |
| | | ARDRRELRAFDIWGQGTMVTVSS | | NWPPWTFGQGTKVEIK |
| clonotype | 3868 | QVQLQQSGPGLVKPSQTLSLTCAI | 3966 | DIVMTQSPDSLAVSLGERATINC |
| 261-1 | | SGDSVSSNSAAWNWIRQSPSRGLE | | KSSQSVLYSSNNKNYLAWYQQKP |
| | | WLGRTYYRSKWYNDYAVSVKSRIT | | GQPPKLLIYWASTRESGVPDRES |
| | | INPDTSKNQFSLQLNSVTPEDTAV | | GSGSGTDFTLTISSLQAEDVAVY |
| | | YYCARGGFFDYWGQGTLVTVSS | | YCQQYYSTPLTFGGGTKVEIK |
| clonotype | 3869 | EVQLVESGGGLVQPGGSLRLSCAA | 3967 | DIQMTQSPSTLSASVGDRVTITC |
| 261-2 | | SGFTFSSYNMHWVRQPTGKGLEWV | | RASQSISRWLAWYQQKPGKAPKL |
| | | SAIGTAGDAYYPGSVKGRFTISRE | | LIYKASSLESGVPSRFSGSGSGT |
| | | NARNSLFLQMNSLRAGDTAVYYCT | | EFTLTVSSLQPDDFATYYCQQYY |
| | | GGSDSSHWYFDIWGRGTLVTVSS | | SYSTFGQGTKLEIK |
| clonotype | 3870 | ELQLVESGGGLVQPGGSLRLSCAA | 3968 | QAVLTQPASLSASPGASASLTCT |
| 262-1 | | SGFTFSSYSMNWVRQAPGKGLEWV | | LRSGINVGTYRIYWYQQKPGSPP |
| | | SYISSSSSTIFYADSVRGRFTISR | | QYLLRYRSDSDKQQGSGVPSRES |
| | | DNAKNSLYLQMNSLRDEDTAVYSC | | GSKDVSANAGILFISGLQSQDEA |
| | | ARRAWGYWYFDLWGRGTLVTVSS | | DYYCMIWYSSASVFGGGTKLTVL |
| clonotype | 3871 | QVQLVQSGTEVKKPGASVKVSCKA | 3969 | SYELTQPLSVSVALGQTARITCG |
| 263-1 | | SGYTFTGYYIHWVRQAPVRQAPGQ | | GNNIGSKNVHWYQQKPGQAPVLV |
| | | GLEWMGWIYPNRGGTSFAQKFQGR | | IYRDSNRPSGIPERFSGSNSGNT |
| | | VTMTRDSSISTAYMELSGLSSDDT | | |
| | | AVYYCARAYSSSWTPLGYWGQGTL | | ATLTISRAQAGDEADYYCQVWDS |
| | | VTVSS | | STAVFGGGTKLTVL |
| clonotype | 3872 | QVQLQESGPGLVKPSETLSLTCTV | 3970 | QSALTQPASVSGSPGQSITISCT |
| 263-2 | | SGGSVSSANYYWTWIRQPPGKGLE | | GTSSDVGGYNYVSWYQQHPGKAP |
| | | WIGYIYYSGSTNYNPSLKSRVSMS | | KLMIYEVSNRPSGVSNRFSGSKS |
| | | VDTSTNQFSLKLSSVTAADTAVYY | | GNTASLTISGLQAEDEADYYCSS |
| | | CARTDFWSGWDYWGQGTLVTVSS | | YTSSSTLVVFGGGTKLTVL |
| clonotype | 3873 | EVQLVESGGGLVQPGGSLRLSCAA | 3971 | SYELTQPPSVSVSPGQTASITCS |
| 264-1 | | SGFTFNTYSVNWVRQAPGKGLEWV | | GDKLGDKYACWYQQKPGQSPVLV |
| | | SYISSGSSTIYYADSVKGRFTVSR | | IYQDSKRPSGIPERFSGSNSGNT |
| | | DNAKNSVYLQMNSLRDEDTAVYYC | | ATLTISGTQAMDEADYYCQAWDS |
| | | ARSGYRYPFHFDYWGQGTLVTVSS | | STAVFGGGTKLTVL |
| clonotype | 3874 | QVQLVQSGAEVKKPGASVQVSCKA | 3972 | SYELTQPPSVSVSPGQTARITCS |
| 264-2 | | SGYTFTNYDINWVRQATGQGLEWM | | GDALPKKYAYWYQQKSGQAPVLV |
| | | GWMNPNSGNTGYAPKFQGRVTMTR | | IYEDSKRPSGIPERFSGSSSGTM |
| | | NTSIRTAYMELSSLRSEDTAVYYC | | ATLTISGAQVEDEADYYCYSTDS |
| | | AIDGLGQLVLFDYWGQGTLVTVSS | | SGNHRVFGGGTKLTVL |
| clonotype | 3875 | EVQLVESGGGLVKPGGSLRLSCAA | 3973 | SYELTQPPSVSVSPGQTARITCS |
| 266-1 | | SGFTFSRYSMNWVRQAPGKGLEWV | | GDALPKKYAYWYQQKSGQAPVLV |
| | | SSISSSSSYIYYADSVKGRFTISR | | IYEDSKRPSGIPERFSGSSSGTM |
| | | DNAKNSLYLQMTSLRAEDTAVFYC | | ATLTISGAQVEDEADYYCYSTDS |
| | | AREGGIAAAGFDYWGQGTLVTVSS | | SGNHWVFGGGTKLTVL |
| clonotype | 3876 | EVQLVESGGNLVQPGGSLRLSCAA | 3974 | SYELTQPPSVSVSPGQTARITCS |
| 267-1 | | SGFTFSSYSMNWVRQAPGKGLEWV | | GDALPKKYAYWYQQKSGQAPVLV |
| | | SYISSSSSTIYYADSVKGRFTISR | | IYEDSKRPSGIPERFSGSSSGTM |
| | | DNAKNSLYLQMNSLRDEDTAVYYC | | ATLTISGAQVEDEADYYCYSTDS |
| | | ARSGYRYPYYFDYWGQGTLVTVSS | | SGNHRVFGGGTKLTVL |
| clonotype | 3877 | EVQLVESGGGLVKPGGSLRLSCAA | 3975 | DIQMTQSPSSLSASVGDRVTITC |
| 267-2 | | SGFTFINAWMSWVRQAPGKGLEWV | | RASQRIGSFLNWYQQKPGKAPKL |
| | | GRIKSETDGGTTDYAAPVKGRFTI | | LIYAASNFQSGVPSRFSGSGSGT |
| | | SRDDSKNTLYLQMNSLKTEDTAVY | | DFTLTISSLQPEDFATYYCQQTY |
| | | YCTTDNWNYEDFDYWDQGTLVTVS | | NTPLTFAGGTKVEIK |
| | | S | | |
| clonotype | 3878 | EVQLVESGGHVIRPGGSLRLSCAA | 3976 | QSALTQPASVSGSPGQSITISCT |
| 268-1 | | SGFTFDDYGMSWVRQVPGKGLEWV | | GTSSDVGGYNYVSWYQQHPGKAP |
| | | SDISWNGGSTGYADSVKGRFTISR | | KLMIYDVSKRPSGVSNRFSGSKS |
| | | DNAKNSLYLQMNSLRAEDTALYYC | | GNTASLTISGLQAEDEADYYCCS |
| | | AREPQQWRYYFDYWGQGTLVTVSS | | YAGSSTWVFGGGTKLTVL |
| clonotype | 3879 | EVQLVESGGGLVQPGGSLRLSCAA | 3977 | QSVLTQPPSVSEAPRQRVTISCS |
| 268-2 | | SGFTFSSYAMSWVRQAPGKGLEWV | | GSSSNIGNNAVNWYQQLPGKAPK |
| | | SAISGSGGSTYYADSVKGRFTISR | | LLIYYDDLLPSGVSDRESGSKSG |
| | | DNSKNTLYLQMNSLRAEDTAVYYC | | TSASLAISGLQSEDEADYYCAAW |
| | | AKARSGIVGATGDYWGQGTLVTVS | | DDRLNGPVFGGGTKLTVL |
| | | S | | |
| clonotype | 3880 | EVQLVESGGGLVKPGGSLRLSCAA | 3978 | DVVMTQSPLSLPVTLGQPASISC |
| 269-1 | | SGFTFSSYSMNWVRQAPGKGLEWV | | RSSQSLVYSDGNTYLNWFQQRPG |
| | | SSISSSSSYIYYADSVKGRFTISR | | QSPRRLIYKVSNRDSGVPDRFSG |
| | | DNAKNSLYLQMNSLRAEDTAVYYC | | SGSGTDFTLKISRVEAEDVGVYY |
| | | ARGGNWNSDAFDIWGQGTMVTVSS | | CMQGTHWPPAFGQGTKLEIK |
| clonotype | 3881 | QVQLVQSGAEVKKPGSSVKVSCKP | 3979 | DIVMTQSPDSLAVSLGERATINC |
| 269-2 | | SGGTFNNYAVTWVRQAPGQGLEWM | | KSSQSVLYSSNNKNYLAWYQQKP |
| | | GGIIPIFGTANYAQKFQGRVTFTA | | GQPPKLLIYWASTRESGVPDRES |
| | | DESTSTAYMELSSLRSEDTAVYYC | | GSGSGTDFTLTISSLQAEDVAVY |
| | | ARDRSGTTSWYFDLWGRGTLVTVS | | YCQQYYSTALTFGGGTKVEIK |
| | | S | | |
| clonotype | 3882 | QVQLQESGPGLVKPSQTLSLTCTV | 3980 | SFDLTQPPSVSVSPGQTASITCS |
| 270-2 | | SGGSISSGGYYWSWIRQHPGKGLE | | GDKLGDKYACWYQRKPGQSPVLV |
| | | WIGYSSYSGSTYYNPSLKSRVIIS | | IYQDSKRPSGIPERFSGSISGNT |
| | | VDTSKNQFSLKLSSVTAADTAVYC | | ATLTISGTQAMDEADYYCQAWDS |
| | | CARYCSSTSCYGFDPWGQGILVTV | | STAVFGGGTKLTVL |
| | | SS | | |
| clonotype | 3883 | EVQLVQSGAEVKKPGESLKISCKG | 3981 | SYELTQPPSVSVSPGQTASITCS |
| 271-1 | | SGYSFTSYWIGWVRQMPGKGLEWM | | GDKLGDKYACWYQQKPGQSPVLV |
| | | GIIYPGDSDTRYSPSFQGQVTISA | | IYQDSKRPSGIPERFSGSNSGNT |
| | | DKSISTAYLQWSSLKASDTAMYYC | | ATLTISGTQAMDEADYYCQAWDS |
| | | ARTEYSSSPEYFQHWGQGTLVTVS | | STALVFGGGTKLTVL |
| | | S | | |
| clonotype | 3884 | QVQLVESGGGLVKPGGSLRLSCAA | 3982 | SYELTQPPSVSVSPGQTASITCS |
| 271-2 | | SGFTFSDYYMSWIRQAPGKGLEWV | | GDKLGDKYACWYQQKPGQSPVLV |
| | | SYISSSGSSIFYADSVKARFTISR | | IYQDSKRPSGIPERFSGSNSGNT |
| | | DNAKNSLYLQVNSLRAEDTAVYYC | | ATLTISGTQAMDEADYYCQAWDS |
| | | AREGFWSGYYDAFDIWGQGTMVTV | | STVVFGGGTKLTVL |
| | | SS | | |
| clonotype | 3885 | QITLKESGPTLVKPTQTLTLTCTF | 3983 | SYELTQPPSVSVSPGQTARITCS |
| 272-1 | | SGFSLSTSGVGVGWIRQPPGKALE | | GDALPKKYAYWYQQKSGQAPVLV |
| | | WLALIYWNDDKRYSPSLKSRLTIT | | IYEDSKRPSGIPERFSGSSSGTM |
| | | KDTSKNQVVLTMTNMDPVDTATYY | | ATLTISGAQVEDEADYYCYSTDS |
| | | CAHRGSGARDAFDIWGQGTMVTVS | | SGNHRVFGGGTKLTVL |
| | | S | | |
| clonotype | 3886 | QVQLQQSGPGLVKPSQTLSLTCAI | 3984 | SSELTQDPAVSVALGQTVRITCQ |
| 272-2 | | SGDSVSSHSGAWNWLRQSPSRGLE | | GDSLRSYYASWYQQKPGQAPVLV |
| | | WLGRTYYRSKWYNDYAVSVKSRIT | | IYGKNNRPSGIPDRFSGSSSGNT |
| | | INPDTSKNQFSLQLNSVTPEDTAV | | ASLTITGAQAEDEADYYCNSRDS |
| | | YYCARDGWTDAFDIWGQGTMVTVS | | SGNHVVFGGGTKLTVL |
| | | S | | |
| clonotype | 3887 | QVQLVQSGAEVKKPGASVKVSCKA | 3985 | SSELTQDPAVSVALGQTVRITCQ |
| 273-1 | | SGYTFTSYYMHWVRQAPGQGLEWM | | GDSLRSYYASWYQQKPGQAPVLV |
| | | GIINPSGGSTSYAQKFQGRVTMTR | | IYGKNNRPSGIPDRFSGSSSGNT |
| | | DTSTSIVYMELSSLRSEDTAVYYC | | ASLTITGAQAEDEADYYCNSRDS |
| | | ARESGYSGYDDFDYWGQGTLVTVS | | SGNHWVFGGGTKLTVL |
| | | S | | |
| clonotype | 3888 | QVQLVESGGGVVQPGRSLRLSCAA | 3986 | DIQMTQSPSTLSASVGDRVTITC |
| 273-2 | | SGFTFSSYGMHWVRQAPGKGLEWV | | RASQSISSWLAWYQQKPGKAPKL |
| | | AVIWYDGSNKYYADSVKGRFTISR | | LIYKASSLESGVPSRFSGSGSGT |
| | | DNSKNTLYLQMNSLRAEDTAVYYC | | EFTLTISSLQPDDFATYYCQQYN |
| | | ARKAVAGYYYYYMDVWGKGTTVTV | | SYWTFGQGTKVEIK |
| | | SS | | |
| clonotype | 3889 | EVQLVESGGGLVQPGGSLRLSCAA | 3987 | SSELTQDPAVSVALGQTVRITCQ |
| 274-1 | | SGFTFSSYSMNWVRQAPGKGLEWV | | GDSLRSYYASWYQQKPGQAPVLV |
| | | SYISSSSYTIYYADSVKGRFTISR | | IYGKNNRPSGIPDRFSGSSSGNT |
| | | DNAKNSLYLQMNSLRDEDTAVYYC | | ASLTITGAQAEDEADYYCNSRDS |
| | | ARDVGGNYYYNMDVWGKGTTVTVS | | SGNHVVFGGGTKLTVL |
| | | S | | |
| clonotype | 3890 | QVQLQESGPGLVKPSGTLSLTCAV | 3988 | ALQMTQSPSSLSASVGDRVTITC |
| 274-2 | | SGGSISSSNWWSWVRQPPGKGLEW | | RASQGIRNDLGWYQQKPGKAPKL |
| | | IGEIYHSGSTNYNPSLKSRVTISV | | LIYTASSLQSGVPSRFSGSGSGT |
| | | DKSKNQFSLKLSSVTAADTAVYYC | | DFTLTISSLQPEDFATYYCLQDY |
| | | ARGQQLVDYYYYMDVWGKGTTVTV | | NYPYTFGQGTKLEIK |
| | | SS | | |
| clonotype | 3891 | EVQVVESGGGLVQPGGSLRLSCAA | 3989 | EIVMTQSPATLSVSPGERATLSC |
| 275-1 | | SGFTFSSYAMSWVRQAPGKGLEWV | | RASQSVSSNLAWYQQKPGQAPRL |
| | | STISGSGGSTYYADSVKGRFTISR | | LIYGASTRATGIPARFSGSGSGT |
| | | DNSKNTLYLQMNSLRAEDTAVYYC | | DFTLTISSLQSEDFAVYYCQQYD |
| | | AKERGTVVYGYFDLWGRGTLVTVS | | NWPRTFGQGTKVEIK |
| | | S | | |
| clonotype | 3892 | EVQLVQSGAEVKKPGESLKISCKG | 3990 | DIVMTQSPLSLPVTPGEPASISC |
| 275-2 | | SGYSFTSYWIGWVRQMPGKGLEWM | | RSSQSLLHSNGYNYLDWYLQKPG |
| | | GIIYPGDSDTRYSPSFQGQVTISA | | QSPQLLIYLGSNRASGVPDRFSG |
| | | DKSISTAYLQWSSLKASDTAMYYC | | SGSGTDFTLKISRVEAEDVGVYY |
| | | ARHRVTGTTFYYMDVWGKGTTVTV | | CMQSLQTPITFGQGTRLEIK |
| | | SS | | |
| clonotype | 3893 | EVQLVQSGAEVKKPGESLKISCKG | 3991 | EIVMTQSPATLSVSPGERATLSC |
| 276-1 | | SGYSFTIYWIGWVRQMPGKGLEWM | | RASQSVSSDLAWYQQKPGQAPRL |
| | | GIIYPGDSDTTYSPSFQGQVTISA | | LIYGATTRATGIPARFSGSGSET |
| | | DKSISTAYLQWTSLKASDTAMYYC | | EFTLTISSLQSEDFAVYYCQQHN |
| | | ARQDRYNWNSPLDFWGQGTLVTVS | | SWPRTFGQGTKVEIK |
| | | S | | |
| clonotype | 3894 | QVQLQESGPGLVKPSQTLSLTCTV | 3992 | DIQMTQSPSSVSASVGDRVPITC |
| 277-1 | | SGGSISSGGYYWSWIRQHPGKGLE | | RASQGISSWLAWYQQKPGKAPKL |
| | | WIGYIYYSGSTYYNPSLKSRVTIS | | LIYAASSLQSGVPSRFSGSGSGT |
| | | VDTSKNQFSLKLSSVTVADTAVYY | | DFTLTISSLQPEDFATYYCQQAN |
| | | CATLRGYTYGPFDYWGQGALVTVS | | SFPYTFGQGTKLEIK |
| | | S | | |
| clonotype | 3895 | QVQLVQSGSELKKPGASMKVSCKA | 3993 | SYELTQPPSVSVSPGQTARITCS |
| 278-2 | | SGYTFTNYAMNWVRQAPGQGLEWM | | GDALPKKYAYWYQQKSGQAPVLV |
| | | GWINTNTGNPTYAQGFTGRFVFSL | | IYEDSKRPSGIPERFSGSSSGTM |
| | | DTSVSTTYLQISSLKAEDTAVYYC | | ATLTISGAQVEDEADYYCYSTDS |
| | | ARDVYSNSHYSYFYMDVWGKGTTV | | SGNLGVFGGGTKLTVL |
| | | TVSS | | |
| clonotype | 3896 | QVQLVQSGSELKKPGASVKVSCKA | 3994 | SYELTQPPSVSVSPGQTASITCS |
| 279-1 | | SGYTFTHNAMNWVRQAPGQGLEWM | | GDKLGDKYACWYQQKPGQSPVLV |
| | | GWINTNTGNPTYAQGFTGREVESL | | IYQDSKRPSGIPERFSGSNSGNT |
| | | DTSVRMTYLQISSLKAEDTAVYYC | | ATLTISGTQAMDEADYYCQAWDS |
| | | ARDSLPDYGDFYLDNWGQGTLVTV | | STVVFGGGTKLTVL |
| | | SS | | |
| clonotype | 3897 | QVQLQQWGAGLLKPSETLSLTCAV | 3995 | EIVMTQSPATLSVSPGERATLSC |
| 279-2 | | YGGSFRGYYWDWIRQPPGKGLEWI | | RASQSVSSNLAWYQQKPGQAPRL |
| | | GEINHGGGTDYNPSLESRVTISVD | | LIYGASTRATGIPARFSGSGSGT |
| | | TSKNQLSLNLNSVTAADTAVYYCV | | EFTLTISSLQSEDFAVYYCQQYN |
| | | RERGYTDAFDIWGQGTMVTVSS | | NWRTFGQGTKVEIK |
| clonotype | 3898 | EVQLVESGGGLVQPGRSLRLSCAA | 3996 | SYELTQPPSVSVSPGQTASITCS |
| 280-1 | | SGFTEDDYAMHWVRQVPGKGLEWV | | GDKLGDKYACWYQQKPGQSPVLV |
| | | SGISWNSDNIGYADSVKGRFTIFR | | IYQDSKRPSGIPERFSGSNSGNT |
| | | DNARNSLHLQMNSLRPEDTALYYC | | ATLTISGTQAMDEADYYCQAWDS |
| | | AKDISHYVGWWYFDIWGRGSLVTV | | STAVFGGGTKLTVL |
| | | SS | | |
| clonotype | 3899 | QVQLVQSGAEVKKPGASVKVSCKA | 3997 | QSALTQPRSVSGSPGQSVTISCT |
| 280-2 | | SGYTFTGYYMHWVRQAPGQGLEWM | | GTSSDVGGYNYVSWYQQHPGKAP |
| | | GWINPNSGGTNYAQKFQGRVTMTR | | KLIIYDVSKRPSGVPDRFSGSKS |
| | | DTSISTAYMELSRLRSDDTAVYYC | | GNTASLTISGLQAEDEADYYCCS |
| | | ARGIAAAGGLYYYYYLDVWGKGTT | | YADNYTWVFGGGTKLTVL |
| | | VTVSS | | |
| clonotype | 3900 | EVQLVESGGGLVQPGGSLKLSCAA | 3998 | SYELTQPPSVSVSPGQTASITCS |
| 281-1 | | SGFTFSGSAMHWVRQASGKGLEWV | | GDKLGDKYACWYQQKPGQSPVLV |
| | | GRIRSKANSYATAYAASVKGRFTI | | IYQDSKRPSGIPERFSGSNSGNT |
| | | SRDDSKNTAYLQMNSLKTEDTAVY | | ATLTISGTQAMDEADYYCQAWDS |
| | | YCTRHLELRGNYFDYWGQGTLVTV | | STVVFGGGTKLTVL |
| | | SS | | |
| clonotype | 3901 | VQLQESGPGLVKPSQTLSLTCTVS | 3999 | SYELTQPPSVSVSPGQTASITCS |
| 281-2 | | GGSINSGGYYWSWIRQHPGKGLEW | | GDKLGDKYACWYQQKPGQSPVLV |
| | | IGYISYIGTTFYNPSLKSRITISV | | IYQDSKRPSGIPERFSGSNSGNT |
| | | DTSKNQFSLKLSSVTAADTAVYYC | | ATLTISGTQAMDEADYYCQAWDS |
| | | ASFYGDYVPHYYFYYMDVWGKGTT | | STVVFGGGTKLTVL |
| | | VTVSS | | |
| clonotype | 3902 | EVHLVESGGGLVQPGGSLRLSCAA | 4000 | SYELTQPPSVSVSPGQTASITCS |
| 282-1 | | SGFSFSDHFMDWVRQAPGKGLEWV | | GHMLGYKYASWYHQKPGQSPVLV |
| | | GHARNKAYSYTTEYAASVKGRFTI | | IYHGTRRPSGIPERFSGSNSGNT |
| | | SRDDSKNSLYLQMNSLKTEDTAVY | | STLTISGTQAMDEADYYCQAWDS |
| | | YCAREGYNWNYDMDVWGKGTTVTV | | TTAVFGGGTKLTVL |
| | | SS | | |
| clonotype | 3903 | QVTLRESGPALVKPTQTLTLTCTF | 4001 | SYELTQPPSVSVSPGQTASITCS |
| 282-2 | | SGFSLSTSGVCVSWIRQPPGKALE | | GDKLGDKYACWYQQKPGQSPVLV |
| | | WLALIDWDDDKYYSTSLKTRLTIS | | IYQDSKRPSGIPERFSGSNSGNT |
| | | KDTSKNQVVLTMTNMDPVDTATYY | | ATLTISGTQAMDEADYYCQAWDS |
| | | CARIREYCTNGVCSYYFDYWGQGT | | STVVFGGGTKLTVL |
| | | LVTVSS | | |
| clonotype | 3904 | QVQLVQSGAEVKKPGASVKVSCKA | 4002 | SSELTQDPAVSVALGQTVRITCQ |
| 283-1 | | SGYTFTSYGISWVRQAPGQGLEWM | | GDSLRSYYASWYQQKPGQAPVLV |
| | | GWISGYNGNTNYVQKLQGRVTMTT | | IYGKNNRPSGIPDRFSGSSSGNT |
| | | DTSTNTAHMELRSLRSDDSAVYYC | | ASLTITGAQAEDEADYYCNSRDS |
| | | ARDPGGYYYSYNMDVWGKGTTVTV | | SGNHWVFGGGTKLTVL |
| | | SS | | |
| clonotype | 3905 | EVQLVESGGGLVKPGGSLRLSCAA | 4003 | SSELTQDPAVSVALGQTVRITCQ |
| 283-2 | | SGFTFSSYSMNWVRQAPGKGLEWV | | GDSLRSYYASWYQQKPGQAPVLV |
| | | SSISSSSSYIYYADSVKGRFTISR | | IYGKNNRPSGIPDRFSGSSSGNT |
| | | DNAKNSLYLQMNSLRAEDTAVYYC | | ASLTITGAQAEDEADYYCNSRDS |
| | | ARGGGYCSSTSCYRYYYYMDVWGK | | SGNHWVFGGGTKLTVL |
| | | GTTVTVSS | | |
| clonotype | 3906 | QVQLVQSGAEVKKPGSSVKVSCKA | 4004 | SSELTQDPAVSVALGQTVRITCQ |
| 284-1 | | SGGTFSSYVITWVRQAPGQGLEWM | | GDSLRSYYASWYQQKPGQAPVLV |
| | | GGIIPIFGTVKYAQKFQGRVKITA | | IYGKNNRPSGIPDRFSGSSSGNT |
| | | DESTSTAYMELSSLRSEDTAVYYC | | ASLTITGAQAEDEADYYCNSRDS |
| | | ARGYCSSTRCYPFDYWGQGTLVIV | | SGNHLVFGGGTKLTVL |
| | | SS | | |
| clonotype | 3907 | EVQLVESGGGLVQPGRSLRLSCEA | 4005 | EIVLTQSPATLSLSPGERATLSC |
| 285-1 | | SGFTFDDYVMHWVRQAPGKGLEWV | | RASQSVSSYLAWYQQKPGQAPRL |
| | | SGISWTSGSVAYADSVKGRFTISR | | LIYDASNRATGIPARFSGSGSGT |
| | | DNAINSLYLQMNSLRAEDTAMYYC | | DFTLTISSLEPEDFAVYYCQQRS |
| | | AKDMGFLEWLSYFDYWGQGTLVTV | | NWPPTFGGGTKVEIK |
| | | SS | | |
| clonotype | 3908 | QIHLVESGGGVVQPGRSLRLSCAA | 4006 | DIQMTQSPSTLSASVGDRVTITC |
| 286-1 | | SGFTFSNYGLHWVRQAPGKGLEWV | | RASQSISSWLAWYQQKPGKAPKL |
| | | AVIWYDGSNKYYADSVKGRFTISR | | LIYKASSLESGVPSRFSGSGSGT |
| | | DNSKNTLYLQMNSLRAEDTAVYYC | | EFTLTITSLQPDDFATYYCQQYN |
| | | ARGPRYNWNYGAFDYWGQGTLVTV | | SFSWTFGQGTKVEIK |
| | | SS | | |
| clonotype | 3909 | QVQLVQSGAEVKKPGASVKVSCKA | 4007 | SYELTQPPSVSVSPGQTASITCS |
| 287-1 | | SGYTFTSYGISWVRQAPGQGLEWM | | GDKLGDKYACWYQQKPGQSPVLV |
| | | GWISAYNGNTNYAQKLQGRVTMTT | | IYQDSKRPSGIPERFSGSNSGNT |
| | | DTSTSTAYMELRSLRSDDTAVYYC | | ATLTISGTQAMDEADYYCQAWDS |
| | | ARGGLELRKLYYYMDVWGKGTTVT | | STVVFGGGTKLTVL |
| | | VSS | | |
| clonotype | 3910 | EVQLVESGGGLVQPGGSLKLSCAA | 4008 | QSALTQPASVSGSPGQSITISCT |
| 288-1 | | SGFTFSGSAMHWVRQASGKGLEWV | | GTSSDVGGYNYVSWYQQHPGKAP |
| | | GRIRSKANSYATAYAASVKGRFTI | | KLMIYDVSKRPSGVSNRFSGSKS |
| | | SRDDSKNTAYLQMNSLKTEDTAVY | | GNTASLTISGLQAEDEADYYCCS |
| | | YCARDGFWSGYDWFDPWGQGTLVT | | YAGSSTWVFGGGTKLTVL |
| | | VSS | | |
| clonotype | 3911 | QVQLVQSGAEVKKPGASVKVSCKA | 4009 | DIVMTQSPDSLAVSLGERATINC |
| 289-1 | | SGYTFPHYGVSWVRQAPGQGLEWM | | KSSQSVLYSSNNKNYLAWYQQKP |
| | | GWISAYNGNTDYAQKLQGRVTMTT | | GQPPKLLIYWASTRESGVPDRES |
| | | DTSTSTAYMELRSLRSDDTAVYYC | | GSGSGTDFTLTISSLQAEDVAVY |
| | | ARGNRDYWDYYYYMDVWGKGTTVT | | YCQQYYSTRTFGQGTKVEIK |
| | | VSS | | |
| clonotype | 3912 | QVQLVQSGSELKKPGASMKVSCKA | 4010 | SSELTQDPAVSVALGQTVRITCQ |
| 290-1 | | SGYTFTNYAMNWVRQAPGQGLEWM | | GDSLRSYYASWYQQKPGQAPVLV |
| | | GWINTNTGNPTYAQGFTGRFVFSL | | IYGKNNRPSGIPDRFSGSSSGNT |
| | | DTSVSTTYLQISSLKAEDTAVYYC | | ASLTITGAQAEDEADYYCNSRDS |
| | | ARDVYSNSHYSYFYMDVWGKGTTV | | SGNHVVFGGGTKLTVL |
| | | TVSS | | |
| clonotype | 3913 | QVQLVQSGAEVKKPGASVKVSCKA | 4011 | SYELTQPPSVSVSPGQTARITCS |
| 292-1 | | SGYTFTSYDIHWVRQATGQGLEWM | | GDALPKKYAYWYQQKSGQAPVLV |
| | | GWMNPNSGNTGYVQKFQGRVTMTR | | IYEDSKRPSGIPERFSGSSSGTM |
| | | NTSTRTAYMELSSLISEDTAVYYC | | ATLTISGAQVEDEADYYCYSTDS |
| | | ARDDFWSGYYKLNGFDIWGQGTMV | | SGNHRVFGGGTKLTVL |
| | | TVSS | | |
| clonotype | 3914 | QVQLQESGPGLVKPSETLSLTCTV | 4012 | QSVLTQPPSASGTPGQRVTISCS |
| 293-1 | | SGDSVSSGSYFWSWIRQPPGKGLE | | GSSSNIGSNTVNWYQQLPGTAPK |
| | | WIGYFYYSGNTNYNPSLKSRVSMS | | LLIYSNNQRPSGVPDRESGSKSG |
| | | VDTSKNQFSLKLSSVTAADTAVYY | | TSASLAISGLQSEDEADYSCAAW |
| | | CARDNWNSHYYYYYMDVWGKGTTV | | DDSLNGYVFGTGTKVTVL |
| | | TVSS | | |
| clonotype | 3915 | QVQLQQWGAGLLKPSETLSLTCAV | 4013 | SYELTQPPSVSVSPGQTARITCS |
| 294-1 | | YGGSFSGYYWSWIRQPPGKGLEWI | | GDALPKKYAYWYQQKSGQAPVLV |
| | | GEINHSGSTNYNPSLKSRVTISVD | | IYEDSKRPSGIPERFSGSSSGTM |
| | | TSKNQFSLKLSSVTAADTAVYYCA | | ATLTISGAQVEDEADYYCYSTDS |
| | | GSWGELLYFDYWGQGTLVTVSS | | SGNHRVFGGGTKLTVL |
| clonotype | 3916 | QVQLQQSGPGLVKPSQTLSLTCAI | 4014 | DIQMTQSPSSLSASVGDRVTITC |
| 295-1 | | SGDSVSSNSAAWNWIRQSPSRGLE | | QASQDISNYLNWYQQKPGKAPKL |
| | | WLGRTYYRSKWYNDYAVSVISRRT | | LIYDASNLETGVPSRFSGSGSGT |
| | | IIPDTSQNQFSLQLNSVTPEDTAV | | DFTFTISSLQPEDIATYYCQQYD |
| | | YYCARGNVGPAGYHFDYWGQGTLV | | NLPYTFGQGTKLEIK |
| | | TVSS | | |
| clonotype | 3917 | QVQLQESGPGLVKPSETLSLTCTV | 4015 | SYELTQPPSVSVSPGQTARITCS |
| 296-1 | | SGGSVSSGTYYWSWIRQPPGKGLE | | GDALPKKYAYWYQQKSGQAPVLV |
| | | WIGYIYYSGSTNYNPSLKSRVTIS | | IYEDSKRPSGIPERFSGSSSGTM |
| | | VDSSKNQFSLKLSSVTAADTAVYY | | ATLTISGAQVEDEADYYCYSTDS |
| | | CARDRSSWYDHYYYYYMDVWGKGT | | SGNHRVFGGGTKLTVL |
| | | TVTVSS | | |
| clonotype | 3918 | EVQLVESGGGLVQPGGSLRLSCAA | 4016 | DIQMTQSPSSLSASVGDRVTITC |
| 299-1 | | SGFTFSSYSMNWVRQAPRKGLEWI | | RASQDISNSLAWFQQKPGKVPRL |
| | | LYISSSSSTIYYADSVKGRFTISR | | LIYAASTLQSGVPSRFSGSGSGT |
| | | DNAKNSLYLQMNSLRDEDTAVYYC | | DFTLTISSLQPEDVATYYCQKYN |
| | | ARGAYDYVWGNYRHDVEDIWGQGT | | SAPWTFGQGTKVEIK |
| | | MVTVSS | | |
| clonotype | 3919 | QITLKESGPTLVKPTQTLTLTCTF | 4017 | SYELTQPPSVSVSPGQTASITCS |
| 300-1 | | SGFSLSTSGVGVGWIRQPPGKALE | | GDKLGDKYACWYQQKPGQSPVLV |
| | | WLALIYWNDDKRYSPSLKSRLTIT | | IYQDSKRPSGIPERFSGSNSGNT |
| | | KDTSKNQVVLTMTNMDPVDTATYY | | ATLTISGTQAMDEADYYCQAWDS |
| | | CAHKYCGGDCYYYYYYYMDVWGKG | | STVVFGGGTKLTVL |
| | | TTVTVSS | | |
| clonotype | 3920 | QLQLQESGPGLVKPSETLSLTCTV | 4018 | QSALTQPRSVSGSPGQSVTISCT |
| 302-1 | | SGGSISSSSYYWGWIRQPPGKGLE | | GTSSDVGGYNYVSWYQQHPGKAP |
| | | WIGSIYYSGSTYYNPSLKSRVTIS | | KLMIYDVSKRPSGVPDRFSGSKS |
| | | VDTSKNQFSLKLSSVTAADTAVYY | | GNTASLTISGLQAEDEADYYCCS |
| | | CARTPITGTTYWYFDLWGRGTLVT | | YAGSYTLVFGGGTKLTVL |
| | | VSS | | |
| clonotype | 3921 | QLQLQESGPGLVKPSETLSLTCTV | 4019 | SYELTQPPSVSVSPGQTARITCS |
| 303-1 | | SGGSISSSSYYWGWIRQPPGKGLE | | GDALPKKYAYWYQQKSGQAPVLV |
| | | WIGTIYFSGSTYYNPSLKSRVTIS | | IYEDSKRPSGIPERFSGSSSGTM |
| | | VDTSKTQFSLKLSSVTAADTALYY | | ATLTISGAQVEDEADYYCYSTDS |
| | | CARKYYDILTGYFNPFDYWGQGTL | | SGNHRVFGGGTKLTVL |
| | | VTVSS | | |

TABLE 14
VH-CDR3 and VL-CDR3 Sequences for EPOR/CD131 Binders II of Table 13

| clonotype_ | VH | VL | | SEQ | | SEQ |
|---|---|---|---|---|---|---|
| id | Gene | Gene | VH-CDR3 AA | ID NO | VL-CDR3 AA | ID NO |
| clonotype | IGH | IGK | CARAGPGIVAAGSFDYW | 4404 | CQQSYSTPLTF | 4502 |
| 6-1 | | | | | | |
| clonotype | IGH | IGL | CARERGAARHYYYYYMD | 4405 | CYSTDSSGNHYVF | 4503 |
| 22-1 | | | VW | | | |
| clonotype | IGH | IGK | CARGPGVYYYYYYMDVW | 4406 | CQQYNNWPITF | 4504 |
| 33-2 | | | | | | |
| clonotype | IGH | IGL | CVREELDHW | 4407 | CLLYYGGAWVF | 4505 |
| 41-2 | | | | | | |
| clonotype | IGH | IGK | CARKRYRSNLDYW | 4408 | CQQYDNLPFTF | 4506 |
| 44-2 | | | | | | |
| clonotype | IGH | IGL | CARARIAAPSEDYW | 4409 | CCSYAGSSTLVF | 4507 |
| 49-2 | | | | | | |
| clonotype | IGH | IGL | CARRAARCFDYW | 4410 | CQSYDSSLSGVVF | 4508 |
| 52-2 | | | | | | |
| clonotype | IGH | IGL | CARGRDYW | 4411 | CQSYDSSLSGWVF | 4509 |
| 54-2 | | | | | | |
| clonotype | IGH | IGK | CARAFDIW | 4412 | CMQGTHWPLTF | 4510 |
| 56-2 | | | | | | |
| clonotype | IGH | IGK | CAREARSSGRGFDYW | 4413 | CMQGTHWPRTF | 4511 |
| 57-2 | | | | | | |
| clonotype | IGH | IGK | CAKDKWELLRGFDYW | 4414 | CQQRSNWYTF | 4512 |
| 61-2 | | | | | | |
| clonotype | IGH | IGL | CARHKAAAGSYYFDYW | 4415 | CYSTDSSGNQVF | 4513 |
| 63-1 | | | | | | |
| clonotype | IGH | IGK | CARDRGRLGGVFDLW | 4416 | CHQSSSLPYTF | 4514 |
| 65-1 | | | | | | |
| clonotype | IGH | IGK | CARKGYSSRWDYW | 4417 | CQQYNSYPITF | 4515 |
| 75-2 | | | | | | |
| clonotype | IGH | IGL | CVREELDHW | 4418 | CLLSYSGAWVF | 4516 |
| 78-1 | | | | | | |
| clonotype | IGH | IGK | CAREGNWGFDYW | 4419 | CQQANSFPYTF | 4517 |
| 80-2 | | | | | | |
| clonotype | IGH | IGK | CARSHNWNYFYFDYW | 4420 | CQQANSFPLTF | 4518 |
| 85-1 | | | | | | |
| clonotype | IGH | IGL | CARAPVPGWFDPW | 4421 | CSSYAGSNNYVE | 4519 |
| 87-1 | | | | | | |
| clonotype | IGH | IGL | CARGMGAVAEDWEDPW | 4422 | CYSTDSSGNHRVE | 4520 |
| 91-2 | | | | | | |
| clonotype | IGH | IGL | CASFITGTW | 4423 | CNSRDSSGNHWVF | 4521 |
| 236-1 | | | | | | |
| clonotype | IGH | IGK | CAKLGFDYW | 4424 | CLQHNSYPYTF | 4522 |
| 237-1 | | | | | | |
| clonotype | IGH | IGL | CAKNLFFDYW | 4425 | CTSYTSTNSVVF | 4523 |
| 243-1 | | | | | | |
| clonotype | IGH | IGL | CARDNWGIDYW | 4426 | CYSTDSSGNHRVF | 4524 |
| 246-1 | | | | | | |
| clonotype | IGH | IGL | CAREVTSFDYW | 4427 | CSSYAGSNNLVF | 4525 |
| 247-1 | | | | | | |
| clonotype | IGH | IGL | CARDNWSSYFDYW | 4428 | CQVWDSSTWVF | 4526 |
| 249-1 | | | | | | |
| clonotype | IGH | IGL | CARGIAARAFAYW | 4429 | CNSRDSSGNHLVF | 4527 |
| 250-1 | | | | | | |
| clonotype | IGH | IGL | CAREGYLDYW | 4430 | CNSRDSSGNHRVE | 4528 |
| 250-2 | | | | | | |
| clonotype | IGH | IGL | CARERNWGGFDYW | 4431 | CQAWDSSTWVF | 4529 |
| 251-1 | | | | | | |
| clonotype | IGH | IGL | CARDYYFYYMDVW | 4432 | CYSTDSSGNHRVF | 4530 |
| 252-1 | | | | | | |
| clonotype | IGH | IGL | CARDSSSWYFDYW | 4433 | CYSTDSSGNHRVF | 4531 |
| 252-2 | | | | | | |
| clonotype | IGH | IGL | CTTVNWAFDYW | 4434 | CNSRDSSGNHWVF | 4532 |
| 253-1 | | | | | | |
| clonotype | IGH | IGI | CVRDNWNYGFDHW | 4435 | CAAWDGRLKVF | 4533 |
| 253-2 | | | | | | |
| clonotype | IGH | IGL | CARGIAAAGLDYW | 4436 | CNSRDSSGNHVVF | 4534 |
| 254-1 | | | | | | |
| clonotype | IGH | IGL | CAREDSRNWEDPW | 4437 | CYSTDSSGNHRVE | 4535 |
| 254-2 | | | | | | |
| clonotype | IGH | IGL | CTSTGTTEDYW | 4438 | CYSTDSSGNHWVF | 4536 |
| 255-1 | | | | | | |
| clonotype | IGH | IGL | CARTSRVAAFDYW | 4439 | CLLSYSGARVE | 4537 |
| 255-2 | | | | | | |
| clonotype | IGH | IGK | CARDDLWITGDYW | 4440 | CLHHNTHPLTF | 4538 |
| 256-1 | | | | | | |
| clonotype | IGH | IGL | CAREEQRNWFDPW | 4441 | CSSYAGSNNLVF | 4539 |
| 256-2 | | | | | | |
| clonotype | IGH | IGL | CARAYNWNGYFDYW | 4442 | CQAWDSSTVVF | 4540 |
| 257-1 | | | | | | |
| clonotype | IGH | IGK | CARDSGDSYAFDLW | 4443 | CQQLDSYPLTF | 4541 |
| 257-2 | | | | | | |
| clonotype | IGH | IGK | CAREGGDYYYYMDVW | 4444 | CQQRSNWPPTF | 4542 |
| 258-1 | | | | | | |
| clonotype | IGH | IGL | CARHDILTGYFDYW | 4445 | CYSTDSSGNHRVF | 4543 |
| 259-1 | | | | | | |
| clonotype | IGH | IGL | CAWYSRDWSDFDYW | 4446 | CYSTDSSGNHRVF | 4544 |
| 259-2 | | | | | | |
| clonotype | IGH | IGK | CARDRRELRAFDIW | 4447 | CQQRSNWPPWTF | 4545 |
| 260-1 | | | | | | |
| clonotype | IGH | IGK | CARGGFFDYW | 4448 | CQQYYSTPLTF | 4546 |
| 261-1 | | | | | | |
| clonotype | IGH | IGK | CTGGSDSSHWYFDIW | 4449 | CQQYYSYSTE | 4547 |
| 261-2 | | | | | | |
| clonotype | IGH | IGL | CARRAWGYWYFDLW | 4450 | CMIWYSSASVF | 4548 |
| 262-1 | | | | | | |
| clonotype | IGH | IGL | CARAYSSSWTPLGYW | 4451 | CQVWDSSTAVE | 4549 |
| 263-1 | | | | | | |
| clonotype | IGH | IGL | CARTDFWSGWDYW | 4452 | CSSYTSSSTLVVE | 4550 |
| 263-2 | | | | | | |
| clonotype | IGH | IGL | CARSGYRYPFHFDYW | 4453 | CQAWDSSTAVE | 4551 |
| 264-1 | | | | | | |
| clonotype | IGH | IGL | CAIDGLGQLVLFDYW | 4454 | CYSTDSSGNHRVF | 4552 |
| 264-2 | | | | | | |
| clonotype | IGH | IGL | CAREGGIAAAGFDYW | 4455 | CYSTDSSGNHWVF | 4553 |
| 266-1 | | | | | | |
| clonotype | IGH | IGL | CARSGYRYPYYFDYW | 4456 | CYSTDSSGNHRVF | 4554 |
| 267-1 | | | | | | |
| clonotype | IGH | IGK | CTTDNWNYEDFDYW | 4457 | CQQTYNTPLTF | 4555 |
| 267-2 | | | | | | |
| clonotype | IGH | IGL | CAREPQQWRYYFDYW | 4458 | CCSYAGSSTWVF | 4556 |
| 268-1 | | | | | | |
| clonotype | IGH | IGL | CAKARSGIVGATGDYW | 4459 | CAAWDDRINGPVF | 4557 |
| 268-2 | | | | | | |
| clonotype | IGH | IGK | CARGGNWNSDAFDIW | 4460 | CMQGTHWPPAF | 4558 |
| 269-1 | | | | | | |
| clonotype | IGH | IGK | CARDRSGTTSWYFDLW | 4461 | CQQYYSTALTF | 4559 |
| 269-2 | | | | | | |
| clonotype | IGH | IGL | CARYCSSTSCYGFDPW | 4462 | CQAWDSSTAVE | 4560 |
| 270-2 | | | | | | |
| clonotype | IGH | IGL | CARTEYSSSPEYFQHW | 4463 | CQAWDSSTALVE | 4561 |
| 271-1 | | | | | | |
| clonotype | IGH | IGL | CAREGFWSGYYDAFDIW | 4464 | CQAWDSSTVVF | 4562 |
| 271-2 | | | | | | |
| clonotype | IGH | IGL | CAHRGSGARDAFDIW | 4465 | CYSTDSSGNHRVF | 4563 |
| 272-1 | | | | | | |
| clonotype | IGH | IGL | CARDGWTDAFDIW | 4466 | CNSRDSSGNHVVF | 4564 |
| 272-2 | | | | | | |
| clonotype | IGH | IGI | CARESGYSGYDDFDYW | 4467 | CNSRDSSGNHWVF | 4565 |
| 273-1 | | | | | | |
| clonotype | IGH | IGK | CARKAVAGYYYYYMDVW | 4468 | CQQYNSYWTF | 4566 |
| 273-2 | | | | | | |
| clonotype | IGH | IGL | CARDVGGNYYYNMDVW | 4469 | CNSRDSSGNHVVF | 4567 |
| 274-1 | | | | | | |
| clonotype | IGH | IGK | CARGQQLVDYYYYMDVW | 4470 | CLQDYNYPYTF | 4568 |
| 274-2 | | | | | | |
| clonotype | IGH | IGK | CAKERGTVVYGYFDLW | 4471 | CQQYDNWPRTF | 4569 |
| 275-1 | | | | | | |
| clonotype | IGH | IGK | CARHRVTGTTFYYMDVW | 4472 | CMQSLQTPITE | 4570 |
| 275-2 | | | | | | |
| clonotype | IGH | IGK | CARQDRYNWNSPLDFW | 4473 | CQQHNSWPRTF | 4571 |
| 276-1 | | | | | | |
| clonotype | IGH | IGK | CATLRGYTYGPFDYW | 4474 | CQQANSFPYTF | 4572 |
| 277-1 | | | | | | |
| clonotype | IGH | IGL | CARDVYSNSHYSYFYMD | 4475 | CYSTDSSGNLGVF | 4573 |
| 278-2 | | | VW | | | |
| clonotype | IGH | IGL | CARDSLPDYGDFYLDNW | 4476 | CQAWDSSTVVF | 4574 |
| 279-1 | | | | | | |
| clonotype | IGH | IGK | CVRERGYTDAFDIW | 4477 | CQQYNNWRTF | 4575 |
| 279-2 | | | | | | |
| clonotype | IGH | IGL | CAKDISHYVGWWYFDIW | 4478 | CQAWDSSTAVE | 4576 |
| 280-1 | | | | | | |
| clonotype | IGH | IGL | CARGIAAAGGLYYYYYL | 4479 | CCSYADNYTWVF | 4577 |
| 280-2 | | | DVW | | | |
| clonotype | IGH | IGL | CTRHLELRGNYFDYW | 4480 | CQAWDSSTVVF | 4578 |
| 281-1 | | | | | | |
| clonotype | IGH | IGL | CASFYGDYVPHYYFYYM | 4481 | CQAWDSSTVVF | 4579 |
| 281-2 | | | DVW | | | |
| clonotype | IGH | IGL | CAREGYNWNYDMDVW | 4482 | CQAWDSTTAVE | 4580 |
| 282-1 | | | | | | |
| clonotype | IGH | IGL | CARIREYCTNGVCSYYF | 4483 | CQAWDSSTVVF | 4581 |
| 282-2 | | | DYW | | | |
| clonotype | IGH | IGL | CARDPGGYYYSYNMDVW | 4484 | CNSRDSSGNHWVF | 4582 |
| 283-1 | | | | | | |
| clonotype | IGH | IGL | CARGGGYCSSTSCYRYY | 4485 | CNSRDSSGNHWVF | 4583 |
| 283-2 | | | YYMDVW | | | |
| clonotype | IGH | IGL | CARGYCSSTRCYPFDYW | 4486 | CNSRDSSGNHLVF | 4584 |
| 284-1 | | | | | | |
| clonotype | IGH | IGK | CAKDMGFLEWLSYFDYW | 4487 | CQQRSNWPPTF | 4585 |
| 285-1 | | | | | | |
| clonotype | IGH | IGK | CARGPRYNWNYGAFDYW | 4488 | CQQYNSFSWTF | 4586 |
| 286-1 | | | | | | |
| clonotype | IGH | IGL | CARGGLELRKLYYYMDV | 4489 | CQAWDSSTVVF | 4587 |
| 287-1 | | | W | | | |
| clonotype | IGH | IGL | CARDGFWSGYDWEDPW | 4490 | CCSYAGSSTWVF | 4588 |
| 288-1 | | | | | | |
| clonotype | IGH | IGK | CARGNRDYWDYYYYMDV | 4491 | CQQYYSTRTF | 4589 |
| 289-1 | | | W | | | |
| clonotype | IGH | IGL | CARDVYSNSHYSYFYMD | 4492 | CNSRDSSGNHVVF | 4590 |
| 290-1 | | | VW | | | |
| clonotype | IGH | IGL | CARDDFWSGYYKLNGFD | 4493 | CYSTDSSGNHRVF | 4591 |
| 292-1 | | | IW | | | |
| clonotype | IGH | IGL | CARDNWNSHYYYYYMDV | 4494 | CAAWDDSLNGYVE | 4592 |
| 293-1 | | | W | | | |
| clonotype | IGH | IGL | CAGSWGELLYFDYW | 4495 | CYSTDSSGNHRVE | 4593 |
| 294-1 | | | | | | |
| clonotype | IGH | IGK | CARGNVGPAGYHFDYW | 4496 | CQQYDNLPYTF | 4594 |
| 295-1 | | | | | | |
| clonotype | IGH | IGL | CARDRSSWYDHYYYYYM | 4497 | CYSTDSSGNHRVF | 4595 |
| 296-1 | | | DVW | | | |
| clonotype | IGH | IGK | CARGAYDYVWGNYRHDV | 4498 | CQKYNSAPWTF | 4596 |
| 299-1 | | | FDIW | | | |
| clonotype | IGH | IGL | CAHKYCGGDCYYYYYYY | 4499 | CQAWDSSTVVF | 4597 |
| 300-1 | | | MDVW | | | |
| clonotype | IGH | IGL | CARTPITGTTYWYFDLW | 4500 | CCSYAGSYTLVF | 4598 |
| 302-1 | | | | | | |
| clonotype | IGH | IGL | CARKYYDILTGYENPFD | 4501 | CYSTDSSGNHRVE | 4599 |
| 303-1 | | | YW | | | |

TABLE 15
Full Heavy Chain (HC) and Light Chain (LC) Sequences for EPOR/CD131 Binders III
(Clonotypes with multiple IGH/IGK/IGL)

| clonotype_ | SEQ | | SEQ | |
|---|---|---|---|---|
| id | ID NO | Full HC AA Sequence | ID NO | Full LC AA Sequence |
| clonotype | 4020 | EVQLVESGGGLVQPGGSLRLSC | 4051 | SYELTQPPSVSVSPGQTASITC |
| 10-2 | | AASGFTFSSYSMNWVRQAPGKG | | SGDKLGDKYACWYQQKPGQSPV |
| | | LEWVSYISSSSSTIYYADSVKG | | LVIYQDSKRPSGIPERFSGSNS |
| | | RFTISRDNADTSLYLQMNSLRD | | GNTATLTISGTQAMDEADYYCQ |
| | | EDTAVYYCARLAARLTEDYWGQ | | AWDSSTAVFGGGTKLTVL |
| | | GTLVTVSS | | |
| | 4021 | QVQLVQSGAEVKKPGASVKVSC | 4052 | QSVLTQPPSVSGAPGQRVTISC |
| | | KVSGYTLTELSIHWVRQAPGKG | | TGSNSNIGADYDVHWYQQLPGT |
| | | LEWMGGFDPEDGETIYAQKFQG | | APKLLIYNNNNRPSGVPDRFSG |
| | | RVTMTEDTSTDTAYMDLSSLRS | | SRSGTSASLAITGLQAEDEAEF |
| | | EDTAMYYCTKGFPGIVGATRWL | | YCQSYDTSLNRYVFGTGTKVTV |
| | | DPWGQGTLVTVSS | | L |
| clonotype | 4022 | EVQLVESGGGLVQPGGSLRLSC | 4053 | QSALTQPASVSGSPGQSITISC |
| 28-2 | | AASGFTFSNYAMSWVRQAPGKG | | TGTSSDVGGYNYVSWYQQHPGK |
| | | LDWVSAISGSGVSTYYADSVKG | | APKLMIYDVSKRPSGVSNRFSG |
| | | RFTISRDNSKTTLYLQMNSLRA | | SKSGNTASLTISGLQAEDEADY |
| | | EDTAVYYCAKSPPGITGTTYYY | | YCCSYAGSSTYVFGTGTKVTVL |
| | | YYMDVWGKGTTVTVSS | | |
| | 4023 | QVQLQESGPGLVKPSETLSLTC | | |
| | | TVSGGSISSYYWSWIRQPPGKG | | |
| | | LEWIGYIYYSGYTNYNPSLKSR | | |
| | | VTISVDTSKNQFSLKLTSVTAA | | |
| | | DTAVYYCARGEQQVGNYYYYYY | | |
| | | MDVWGKGTTVTVSS | | |
| clonotype | 4024 | QVQLVQSGAEVKKPGASVKVSC | 4054 | SYELTQPPSVSVSPGQTARITC |
| 35-2 | | KASGYTFTSYGISWVRQAPGQG | | SGDALPKKYAYWYQQKSGQAPV |
| | | LEWMGWISGYNGNTNYVQKLQG | | LVIYEDSKRPSGIPERFSGSSS |
| | | RVTMTTDTSTNTGHMELRSLRS | | GTMATLTISGAQVEDEADYYCY |
| | | DDSAVYYCARDPGGYYYSYNMD | | STDSSGNHSVFGTGTKVTVL |
| | | VWGKGTTVTVSS | | |
| | | | 4055 | SYELTQPPSVSVSPGQTARITC |
| | | | | SGDALPKKYAYWYQQKSGQAPV |
| | | | | LVIYEDSKRPSGIPERFSGSKS |
| | | | | GTMATLTISGAQVEDEADYYCY |
| | | | | STFSSDNYRVFGGGTKLTVL |
| clonotype | 4025 | QVQLVESGGGLVKPGGSLRLSC | 4056 | SYELTQPPSVSVSPGQTARITC |
| 43-1 | | AASGFTFSDYYMSWIRQAPGKG | | SADALPKQYAYWYQQKPGQAPV |
| | | LEWVSYISGTGNTIYYADSVKG | | LVIYKDSERPSGIPERFSGSSS |
| | | RFTISRDNAINSLYLQMNSLRA | | GTTVTLTISGVQAEDEADYYCQ |
| | | EDTALYYCARERNWGGFDYWGQ | | SADSSGTYPVVFGGGTKLTVL |
| | | GTLVTVSS | | |
| | | | 4057 | SYELTQPPSVSVSPGQTARITC |
| | | | | SGDALPKKYAYWYQQKSGQAPV |
| | | | | LVIYEDSKRPSGIPERFSGSSS |
| | | | | GTMATLTISGAQVEDEADYYCY |
| | | | | STDSSGNHVVFGGGTKLTVL |
| clonotype | 4026 | EVQLVESGGGLVQPGGSLRLSC | 4058 | DIQMTQSPSTLSASVGDRVTIT |
| 73-2 | | AASGFTFSSYAMSWVRQAPGKG | | CRASQSISSWLAWYQQKPGKAP |
| | | LEWVSAISGSGGSTYYADSVKG | | KLLIYKASSLESGVPSRFSGSG |
| | | RFTISRDNSKNTLYLQMNSLRA | | SGTEFTLTISSLQPDDFATYYC |
| | | EDTAVYYCAKDRAYYDSSGWDY | | QQYNSYYTFGQGTKLEIK |
| | | WGQGTLVTVSS | | |
| | 4027 | EVQLVESGGGVVRPGGSLRLSC | | |
| | | AASGFTFSSYAMSWVRQAPGKG | | |
| | | LEWVSGINWNGGSTGYVDSVKG | | |
| | | RFTISRDNAKNSLYLQMNSLRA | | |
| | | EDTALYYCARERRFLQWLEGYS | | |
| | | YYYMDVWGKGTTVTVSS | | |
| clonotype | 4028 | EVQLVESGGGLVQPGGSLRLSC | 4059 | DVVMTQSPLSLPVTLGQPASIS |
| 77-2 | | AASGFTFSSYSMNWVRQAPGKG | | CRSSQSLVYSDGNTYLNWFQQR |
| | | LEWVSYISSSSSTIYYADSVKG | | PGQSPRRLIYKVSNRDSGVPDR |
| | | RFTISRDNAKNSLYLQMNSLRD | | FSGSGSGTDFTLKISRVEAEDV |
| | | EDTAVYYCASLAYCGGDCYSDF | | GVYYCMQGTHWPPFTFGPGTKV |
| | | DYWGQGTLVTVSS | | DIK |
| | | | 4060 | DIQLTQSPSFLSASVGDRVTIT |
| | | | | CRASQGISSYLAWYQQKPGKAP |
| | | | | KLLIYAASTLQSGVPSRFSGSG |
| | | | | SGTEFTLTISSLQPEDFATYYC |
| | | | | QQLNSYPFTFGPGTKVDIK |
| clonotype | 4029 | EVKLVESGGGLVQPGRSLRLSC | 4061 | DIQMTQSPSSLSASVGDRVTIT |
| 82-2 | | AASGFTFDDYAMYWVRQAPGKG | | CQASQDISNYLNWYQQKPGKAP |
| | | LQWVSGISWSSNNIGYADSVKG | | KLLIYDASNLETGVPSRFSGSG |
| | | RFTISRDNAKNSLYLQMNSLRD | | SGTDFTFTISSLQPEDIATYYC |
| | | EDTAWYYCAKGSTLSTKDWFDP | | QQYDNLPITFGQGTRLEIK |
| | | WGQGTLVTVSS | | |
| | | | 4062 | QSALTQPASVSGSPGQSITISC |
| | | | | TGTSSDVGGYNYVSWYQQHPGK |
| | | | | APKLMIYDVSKRPSGVSNRFSG |
| | | | | SKSGSTASLTISGLQAEDATDY |
| | | | | YCCSYAGSSTWVFGGGTRLTVL |
| clonotype | 4030 | EVQLVESGGGLVQPGRSLRLSC | 4063 | DIVMTQTPLSSPVTLGQPASIS |
| 87-2 | | AASGFTFGDYAIHWVRQGPGKG | | CRSSQSLVHSDGNTYLSWLQQR |
| | | LEWVSGITWNSGSTGYADSVKG | | PGQPPRLLIYKISNRFSGVPDR |
| | | RFTISRDNAKNSLYLQMNSLRS | | FSGSGAGTDFTLKISRVEAEDV |
| | | EDTALYYCAKGIWFGELFPLDY | | GVYYCMQATQFPYTFGQGTKLE |
| | | WGQGTLVTVSS | | IK |
| | | | 4064 | EIVLTQSPGTLSLSPGERASLS |
| | | | | CRASQSFISSYLAWYLQKPGQA |
| | | | | PRLLIYGASSRATGIPDRESGS |
| | | | | GSGTDFTLTISRLEPEDFAMYY |
| | | | | CQQSSRSPYTFGQGTYLEIK |
| clonotype | 4031 | QVQLVQSGAEVKKPGASVKVSC | 4065 | DIVMTQSPDSLAVSLGERATIN |
| 244-1 | | KASGYTFTSYGISWVRQAPGQG | | CKSSQSVLYSSNNKNYLAWYQQ |
| | | LEWMGWISAYNGNTNYAQKLQD | | KPGQPPKLLIYWASTRESGVPD |
| | | RVTMTTDTSTSTAYMDLRSLRS | | RFSGSGSGTDFTLTISSLQAED |
| | | GDTAVYYCARGRYFDLWGRGTL | | VAVYYCQQYYSTPLTFGGGTKV |
| | | VTVSS | | EIK |
| | 4032 | QVHLQESGPGLVKPSQTLSLTC | | |
| | | TVSGGSISSGGYFWTWIRRHPG | | |
| | | KGLEWIGYISYSGSTYYTPSLK | | |
| | | SRLTLSVDTSKNQFSLKLSSLT | | |
| | | AADTAMYYCARTGIPGTTMAFD | | |
| | | IWGQGTMVTVSS | | |
| clonotype | 4033 | EVQLVESGGGLIQPGGSLRLSC | 4066 | SYVLTQPPSVSVAPGKTARITC |
| 251-2 | | AASGFTVISNYMSWVRQAPGKG | | GGNNIGSKSVHWYQQKPGQAPV |
| | | LEWVSVIYSDGHTDYADSVKGR | | LVIYYDSDRPSGIPERFSGSNS |
| | | FTISRDNSKNTLYLQMNSLRAE | | GNTATLTISRVEAGDEADYYCQ |
| | | DTAVYYCARENSNYFDYWGQGT | | VWDSSSDVVFGGGTKLTVL |
| | | LVTVSS | | |
| | 4034 | EVQLVESGGGLVQPGGSLRLSC | | |
| | | AASGFTFSDYSMNWVRQAPGNG | | |
| | | LEWVSYISSFSSNIYYADSVKG | | |
| | | RFTISRDNAKNSLFLQMNGLRD | | |
| | | EDTAIYYCARVYRVGAFDYWGQ | | |
| | | GTLVTVSS | | |
| clonotype | 4035 | QVQLVQSGAEVKKPGASVKVSC | 4067 | SSELTQDPAVSVALGQTVRITC |
| 258-2 | | KASGYTFTRYGINWVRQAPGQG | | QGDSLRSYYASWYQQKPGQAPV |
| | | LEWMGWISTYNGNTKYEQKIQG | | LVIYGKNNRPSGIPDRESGSSS |
| | | RVTMTTDTSTSTAYMDLRSLRS | | GNTASLTITGAQAEDEADYYCN |
| | | DDTAVYYCARDKPGGAGFDPWG | | SRDSSGNHVVFGGGTKLTVL |
| | | QGTLVTVSS | | |
| | 4036 | EVQLVESGGGLVQPGGSLRLSC | 4068 | SYELTQPLSVSVALGQTARITC |
| | | AASGFTFSNYWMHWVRQAPGKG | | GGNNIGSKNVHWYQQKPGQAPV |
| | | LVWVSRINSDGSTADSVKGRFT | | LVIYRIRNRPSGIPERFSGSNS |
| | | ISRDNAKNTLYLQMNSLRAEDS | | GNTATLTISRVQVGDEADYYCQ |
| | | AVYYCAREGTTLVTDAFDIWGQ | | VWDSSTWVFGGGTKLTVL |
| | | GTMVTVSS | | |
| clonotype | 4037 | QVQLVQSGSELKKPGASVKVSC | 4069 | DIQMTQSPSSLSASVGDRVTIT |
| 260-2 | | KASGYTFTNYALNWVRQAPGQG | | CRASQGISNYLAWFQQKPGKTP |
| | | LEWMGWINTNSGNPRYVQGFTG | | KSLIFAASSLQSGVPSKFSGSG |
| | | RFVFSLDTSVSTAYLQISSLKA | | SGTDFTLTISSLQPEDFATYYC |
| | | EDTAVYYCARDSSWFPYFDYWG | | HQYNSYPFTFGPGTKVDIK |
| | | QGTLVTVSS | | |
| | | | 4070 | EIVLTQSPATLSLSPGERAALS |
| | | | | CRASQSVSYYLAWYQQKPGQAP |
| | | | | RVLIYDTSNRATGIPARFSGSG |
| | | | | SGTDFTLTISSLEPEDFAVYYC |
| | | | | QQRSNWPWTFGQGTKVEIK |
| clonotype | 4038 | QVQLVESGGGVVQPGRSLRLSC | 4071 | DIVMTQTPLSSPVTLGQPASIS |
| 262-2 | | AASGFIFSNYGMHWVRQAPGKG | | CRSSQSLVYSDGNTYLSWLQQR |
| | | LEWVTVILYDGNNKYYADSVKG | | PGQPPRLLIYKISNRFSGVPDR |
| | | RFTISRDNSKNTLYLQMNSLRV | | FSGSGAGTDFTLKISRVEAEDV |
| | | EDTAVYYCAKPGPRGGQFDYWG | | GVYYCMQATQFPYTFGQGTKLE |
| | | QGTLVTVSS | | IK |
| | | | 4072 | EIVLTQSPGTLSLSPGERATLS |
| | | | | CRASQNISFSYLAWYQQKPGQA |
| | | | | PRLLIYGESSRATGIPDRFSGS |
| | | | | GSGTDFTLTISRLEPEDFAVYC |
| | | | | CQQYVTHLRTFGGGTKVEIK |
| clonotype | 4039 | QVQLQESGPGLVKPSGTLSLIC | 4073 | SYELTQPPSVSVSPGQTASITC |
| 265-1 | | GVSGGSISRSNWWSWVRQPPGK | | SGDKLGDKYACWYQQKPGQSPV |
| | | GLEWIGEIYHSGSTNYKPSLKS | | LVIYQDSKRPSGIPERFSGSNS |
| | | RVTISVDKSKNQFSLKLSSVTA | | GNTATLTISGTQAMDEADYYCQ |
| | | ADTAVYWCARGATGDGAGFDYW | | AWDSSTAVVFGGGTKLTVL |
| | | GQGTLVTVSS | | |
| | | | 4074 | QSALTQPPSASGSPGQSVTISC |
| | | | | TGTSSDVGGYNYVSWYQQHPGK |
| | | | | APKLMIYEVSKRPSGVPDRFSG |
| | | | | SKSGNTASLTVSGLQAEDEADY |
| | | | | YCSSYAGSNNLVFGGGTKLTVL |
| clonotype | 4040 | QVHLVQSGAEVKKPGASVKVSC | 4075 | QTVVTQEPSLTVSPGGTVTLTC |
| 266-2 | | KASGDTFTSHYIHWVRQAPGQG | | ASSTGAVTSGYYPNWFQQKPGQ |
| | | LEWMGIINPRGGGTSSSQKFQG | | APRALIYSTSNKHSWTPARFSG |
| | | RVTMTRDTSTYTVYMELTSLRS | | SLLGGKAALTLSGVQPEDEAEY |
| | | EDTAVYYCARDGFWNGYDWFDP | | YCLLYYGGAYVFGTGTKVTVL |
| | | WGQGTLVTVSS | | |
| | | | 4076 | SSELTQDPAVSVALGQTVRITC |
| | | | | QGDSLRNYYASWYQQKPGQAPV |
| | | | | LVIYAKNNRPSGIPDRFSGSSS |
| | | | | GNTASLTITGAQAEDEADFYCN |
| | | | | SRDSSGNRMFGGGTKLTVL |
| clonotype | 4041 | QVQLVQSGAEVKKPGASVKVSC | 4077 | SYELTQPPSVSVSPGQTASITC |
| 270-1 | | KASGYTFTSYGISWVRQAPGQG | | SGDKLGDKYACWYQQKPGQSPV |
| | | LEWMGWISGYNGNTNYVQKLQG | | LVIYQDSKRPSGIPERFSGSNS |
| | | RVTMTTDTSTNTAHMELRSLRS | | GNTATLTISGTQAMDEADYYCQ |
| | | DDSAVYFCARDPGGYYYSYNMD | | AWDSSTVVFGGGTKLTVL |
| | | VWGKGTTVTVSS | | |
| | 4042 | EVQLVESGGGLVQPGGSLRLSC | | |
| | | AASGFTFSNYWMHWVRQAPGKG | | |
| | | LVWVSRINSDGSTADSVKGRFT | | |
| | | ISRDNAKNTLYLQMNSLRAEDS | | |
| | | AVYYCAREGTTLVTDAFDIWGQ | | |
| | | GTMVTVSS | | |
| clonotype | 4043 | EVQLVESGGGLVQPGGSLRLSC | 4078 | DIQMTQSPSTLSASVGDRVTIT |
| 276-2 | | AASGFTFSSYDMHWVRQATGKG | | CRASQSISSWLAWYQQKPGKAP |
| | | LEWVSAIGTAGDTYYPGSVKGR | | KLLIYKASSLESGVPSRFSGSG |
| | | FTISRENAKNSLYLQMNSLRAG | | SGTEFTLTISSLQPDDFATYYC |
| | | DTAVYYCARGGAARPYYYYYMD | | QQYNSYWTFGQGTKVEIK |
| | | VWGKGTTVTVSS | | |
| | 4044 | EVQLVESGGGLVQPGGSLRLSC | | |
| | | AASGFTFSSYDMHWVRQATGKG | | |
| | | LEWVSAIGTAGDTYYPGSVKGR | | |
| | | FTISRENAKNSLYLQMNSLRAG | | |
| | | DTAVYYCARGGAARPYYYYYYM | | |
| | | DVWGKGTTVTVSS | | |
| clonotype | 4045 | QVQLVQSGSELKKPGASVKVSC | 4079 | SSELTQDPAVSVALGQTVRITC |
| 277-2 | | KASGYTFTSYAMNWVRQAPGQG | | QGDSLRSYYASWYQQKPGQAPV |
| | | LEWMGWINTNTGNPTYAQGFTG | | LVIYGKNNRPSGIPDRESGSSS |
| | | RFVFSLDTSVSTAYLQISSLKA | | GNTASLTITGAQAEDEADYYCN |
| | | EDTAVYYCARERVGATDYYYYY | | SRDSSGNHWVFGGGTKLTVL |
| | | MDVWGKGTTVTVSS | | |
| | | | 4080 | SYELTQPPSVSVSPGQTASITC |
| | | | | SGDKLGDKYACWYQQKPGQSPV |
| | | | | LVIYQDSKRPSGIPERFSGSNS |
| | | | | GNTATLTISGTQAMDEADYYCQ |
| | | | | AWDSSTAVFGGGTKLTVL |
| clonotype | 4046 | QVQLVQSGSELKKPGASVKVSC | 4081 | QSALTQPASVSGSPGQSIAFSC |
| 291-1 | | KASGYTFTNYAMNWVRQAPGQG | | TGTSSDVGSYDYVSWYQQHPGK |
| | | LEWMGWINTNTGNPTYAQGFTG | | APKLMIYDVSKRPSGLSNRFSG |
| | | RFVFSLDTSVSTAYLQISSLKA | | SKSGNTASLTISGLQAEDEADY |
| | | EDTAVYYCARDGWELLGYYHYY | | YCCSYTSSSTWVFGGGTKLTVL |
| | | MDVWGKGTTVTVSS | | |
| | | | 4082 | SYELTQPPSVSVSPGQTARITC |
| | | | | SGDALPKKYAYWYQQKSGQAPV |
| | | | | LVIYEDSKRPSGIPERFSGSSS |
| | | | | GTMATLTISGAQVEDEADYYCY |
| | | | | STDSSGNHRVFGGGTKLTVL |
| clonotype | 4047 | QVQLQQWGAGLLKPSETLSLTC | 4083 | SSELTQDPAVSVALGQTVRITC |
| 297-1 | | AVYGGSFSGYYWSWIRQPPGKG | | QGDSLRSYYASWYQQKPGQAPV |
| | | LEWIGEINHSGSTNYNPSLKSR | | LVIYGKNNRPSGIPDRFSGSSS |
| | | VTISVDTSKNQFSLKLSSVTAA | | GNTASLTITGAQAEDEADYYCN |
| | | DTAVYYCASRIAAAALLFDYWG | | SRDSSGNHLVFGGGTKLTVL |
| | | QGTLVTVSS | | |
| | | | 4084 | SYELTQPHSVSVATAQMARITC |
| | | | | GGNNIGSKAVHWYQQKPGQDPV |
| | | | | LVIYSDSNRPSGIPERFSGSNP |
| | | | | GNTATLTISRIEAGDEADYYCQ |
| | | | | VWDSSSDHRVFGGGTKLTVL |
| clonotype | 4048 | QVQLQQSGPGLVKPSQTLSLTC | 4085 | SSELTQDPAVSVALGQTVRITC |
| 298-1 | | AISGDSVSSNSAAWNWIRQSPS | | QGDSLRSYYASWYQQKPGQAPV |
| | | RGLEWLGRTYYRSKWYNDYAVS | | LVIYGKNNRPSGIPDRESGSSS |
| | | VKSRITINPDTSKNQFSLQLNS | | GNTASLTITGAQAEDEADYYCN |
| | | VTPEDTAVYYCARACSSTSCYG | | SRDSSGNHVVFGGGTKLTVL |
| | | AFDIWGQGTMVTVSS | | |
| | 4049 | QLQLQESGPGLVKPSATLSLTC | | |
| | | TVSGGSISSSNYYWGWIRQPPG | | |
| | | KGLEWIGSIYYSGSTYYNPSLK | | |
| | | SRVTISVDTSKNQFSLKLSSVT | | |
| | | AADTAVYYCARQMYNWNDGGFD | | |
| | | PWGQGTLVTVSS | | |
| clonotype | 4050 | EVQLVESGGGLVKPGGSLRLSC | 4086 | SYELTQPPSVSVSPGQTARITC |
| 301-1 | | AASGFTFSSYSMNWVRQAPGKG | | SADALPKQYAYWYQQKPGQAPV |
| | | LEWVSSISSNSSYIFYADSVKG | | LVIYKDSERPSGIPERFSGSSS |
| | | RFTISRDNAKNSLYLQMNSLRA | | GTTVTLTISGVQAEDEADYYCQ |
| | | EDTAVYYCARDGFRYYYDSGGY | | SADSSGTYWVFGGGTKLTVL |
| | | YSNWFDPWGQGTLVTVSS | | |
| | | | 4087 | SYELTQPPSVSVSPGQTARITC |
| | | | | SGDALPKKYAYWYQQKSGQAPV |
| | | | | LVIYEDSKRPSGIPERFSGSSS |
| | | | | GTMATLTISGAQVEDEADYYCY |
| | | | | STDSSGNHRVFGGGTKLTVL |

TABLE 16
VH-CDR3 and VL-CDR3 Sequences for EPOR/CD131 Binders III (Clonotypes with
multiple IGH/IGK/IGL) of Table 15

| clonotype | VH | VL | | SEQ | | SEQ |
|---|---|---|---|---|---|---|
| id | gene | gene | VH-CDR3 AA | ID NO | VL-CDR3 AA | ID NO |
| clonotype | IGH | IGL | CARLAARLTFDYW | 4600 | CQAWDSSTAVF | 4631 |
| 10-2 | IGH | IGL | CTKGFPGIVGATRWLDPW | 4601 | CQSYDTSLNRYVF | 4632 |
| clonotype | IGH | IGL | CAKSPPGITGTTYYYYYMDVW | 4602 | CCSYAGSSTYVF | 4633 |
| 28-2 | IGH | | CARGEQQVGNYYYYYYMDVW | 4603 | | |
| clonotype | IGH | IGL | CARDPGGYYYSYNMDVW | 4604 | CYSTDSSGNHSVF | 4634 |
| 35-2 | | IGL | | | CYSTFSSDNYRVF | 4635 |
| clonotype | IGH | IGL | CARERNWGGFDYW | 4605 | CQSADSSGTYPVVF | 4636 |
| 43-1 | | IGL | | | CYSTDSSGNHVVF | 4637 |
| clonotype | IGH | IGK | CAKDRAYYDSSGWDYW | 4606 | CQQYNSYYTF | 4638 |
| 73-2 | IGH | | CARERRFLQWLEGYSYYYMDVW | 4607 | | |
| clonotype | IGH | IGK | CASLAYCGGDCYSDFDYW | 4608 | CMQGTHWPPFTF | 4639 |
| 77-2 | | IGK | | | CQQLNSYPFTF | 4640 |
| clonotype | IGH | IGK | CAKGSTLSTKDWFDPW | 4609 | CQQYDNLPITF | 4641 |
| 82-2 | | IGL | | | CCSYAGSSTWVF | 4642 |
| clonotype | IGH | IGK | CAKGIWFGELFPLDYW | 4610 | CMQATQFPYTF | 4643 |
| 87-2 | | IGK | | | CQQSSRSPYTF | 4644 |
| clonotype | IGH | IGK | CARGRYFDLW | 4611 | CQQYYSTPLTF | 4645 |
| 244-1 | IGH | | CARTGIPGTTMAFDIW | 4612 | | |
| clonotype | IGH | IGL | CARENSNYFDYW | 4613 | CQVWDSSSDVVF | 4646 |
| 251-2 | IGH | | CARVYRVGAFDYW | 4614 | | |
| clonotype | IGH | IGL | CARDKPGGAGFDPW | 4615 | CNSRDSSGNHVVF | 4647 |
| 258-2 | IGH | IGL | CAREGTTLVTDAFDIW | 4616 | CQVWDSSTWVF | 4648 |
| clonotype | IGH | IGK | CARDSSWFPYFDYW | 4617 | CHQYNSYPFTF | 4649 |
| 260-2 | | IGK | | | CQQRSNWPWTF | 4650 |
| clonotype | IGH | IGK | CAKPGPRGGQFDYW | 4618 | CMQATQFPYTE | 4651 |
| 262-2 | | IGK | | | CQQYVTHLRTF | 4652 |
| clonotype | IGH | IGL | CARGATGDGAGFDYW | 4619 | CQAWDSSTAVVF | 4653 |
| 265-1 | | IGL | | | CSSYAGSNNLVF | 4654 |
| clonotype | IGH | IGL | CARDGFWNGYDWFDPW | 4620 | CLLYYGGAYVE | 4655 |
| 266-2 | | IGL | | | CNSRDSSGNRMF | 4656 |
| clonotype | IGH | IGL | CARDPGGYYYSYNMDVW | 4621 | CQAWDSSTVVF | 4657 |
| 270-1 | IGH | | CAREGTTLVTDAFDIW | 4622 | | |
| clonotype | IGH | IGK | CARGGAARPYYYYYMDVW | 4623 | CQQYNSYWTF | 4658 |
| 276-2 | IGH | | CARGGAARPYYYYYYMDVW | 4624 | | |
| clonotype | IGH | IGL | CARERVGATDYYYYYMDVW | 4625 | CNSRDSSGNHWVF | 4659 |
| 277-2 | | IGL | | | CQAWDSSTAVF | 4660 |
| clonotype | IGH | IGL | CARDGWELLGYYHYYMDVW | 4626 | CCSYTSSSTWVF | 4661 |
| 291-1 | | IGL | | | CYSTDSSGNHRVF | 4662 |
| clonotype | IGH | IGL | CASRIAAAALLFDYW | 4627 | CNSRDSSGNHLVF | 4663 |
| 297-1 | | IGL | | | CQVWDSSSDHRVF | 4664 |
| clonotype | IGH | IGL | CARACSSTSCYGAFDIW | 4628 | CNSRDSSGNHVVF | 4665 |
| 298-1 | IGH | | CARQMYNWNDGGFDPW | 4629 | | |
| clonotype | IGH | IGL | CARDGFRYYYDSGGYYSNWFDPW | 4630 | CQSADSSGTYWVF | 4666 |
| 301-1 | | IGL | | | CYSTDSSGNHRVF | 4667 |

TABLE 17
VH and VL Sequences for EPOR/CD131 Binders IV (Phage Library)

| Clone | SEQ | | SEQ | |
|---|---|---|---|---|
| ID | ID NO | VH Sequence | ID NO | VL Sequence |
| 06C01 | 4088 | EVQLVQSGAEVKKPGASVKVSCKASG | 4246 | AIRMTQSPSSVSASVGDRVTIT |
| | | YTFTGNYIHWVRQVPGHGLEWMGWIN | | CRASQGIRGWLAWYQQKPGKAP |
| | | PYSDATHYAQKFQGRVTLTRDTSINT | | NLLIYSASSLQSGVPSRFSGSG |
| | | AYMQLSRLTSDDTAVYYCARAARPDW | | SGTDFTLTISSLQPEDFATYYC |
| | | FDPWGQGTLVTVSS | | QQSYSTPLTFGGGTKVEIK |
| 06H04 | 4089 | QVQLVQSGAEVRKPGASVKVSCKASG | 4247 | DIQLTQSPSTLSASVGDRVTIT |
| | | YTFTDNYIHWVRQVPGQGLEWMGWIN | | CRASQSISSWLAWYQQKPGKAP |
| | | PYSGATHYAQNFQDRITMTRDTSINT | | KLLIYKASSLESGVPSRFSGSG |
| | | AYMHLSRLTSDDTTVYYCARAARPDW | | SGTDFTLTIANLQPEDFATYYC |
| | | FDPWGQGTLVTVSS | | QQSYSSPLTFGGGTKVEIK |
| 10H09 | 4090 | QVQLVQSGAEVKKPGASVKVSCKASG | 4248 | QSVLTQPPSVSGAPGQRVTISC |
| | | YTFTNYYMHWVRQAPGQGLEWLGWII | | TGSSSNIGAGYDVHWYQQLPGT |
| | | PNSGGTKFAQKFQGRVTMTRDTSINT | | APKLLIYGNSNRPSGVPDRFSG |
| | | AYMELSRLRSDDTALYYCARAAYSSS | | SKSGTSASLAITGLQAEDEADY |
| | | WYDYWGQGTLVTVSS | | YCQSYDSSLSGWVFGGGTQLTV |
| | | | | L |
| 03G03 | 4091 | QVQLVQSGAEVKKPGASVKVSCKASG | 4249 | DIQMTQSPSSLSASVGDRVTIT |
| | | YTFTNHFMHWVRQAPGQGLEWMGIIN | | CRASQGIRNDLGWYQQKPGKAP |
| | | PSGGSTNYAQKFQGRVTMTRDTSTSA | | KLLIYAASSLQSGVPSRFSGSG |
| | | VYMELSSLRSEDTAVYYCAAADGYEA | | SGTDFTLTISGLQPEDFATYYC |
| | | FDIWGQGTMVTVSS | | LQDYNYPFTFGPGTKVDIK |
| 11F05 | 4092 | QVQLVQSGAEVKKPGASMKVSCKVSG | 4250 | QSVLTQPPSVSGAPGQRVTISC |
| | | YTFTGYYIHWVRQAPGQGLEWMGWII | | TGSSSNIGAGYDVHWYQQLPGT |
| | | PNSGGTKYAQKFQGRVTLTRDTSIST | | APKLLIYGNSNRPSGVPDRFSG |
| | | AYMDLSRLRSDDTAIYFCARAGFSSS | | STSGTSASLAITGLQAEDEADY |
| | | WYDYWGQGTLVTVSS | | YCQSYDSSLSGWVFGGGTKLTV |
| | | | | L |
| 07H07 | 4093 | QMQLVQSGAEVKKPGASVKVSCKASG | 4251 | QSVVTQPPSVSGAPGQRVTISC |
| | | YTFTGYYMHWVRQAPGQGLEWMGWIN | | TGSSSNIGAGYDVHWYQQLPGT |
| | | PHSGGTNYAQKFQGRVTMTRDTSINT | | APKLLIYGNSNRPSGVPDRFSG |
| | | AYMELSRLRSDDTAVYYCARAGICSG | | SKSGTSASLAITGLQAEDEADY |
| | | TTCYGYGSDYWGQGTTVTVSS | | YCQSYDSRLSGNWVFGGGTKVT |
| | | | | VL |
| 08D01 | 4094 | EVQLVQSGAEVKKPGASVKVSCKASG | 4252 | QSVLTQPPSVSGAPGQRVTISC |
| | | YTFTGYYMHWVRQAPGQGLEWMGWIN | | TGSSSNIGAGYDVHWYQQLPGT |
| | | PHSGGTNYAQKFQGRVTMTRDTSINT | | APKLLIYGNSNRPSGVPDRFSG |
| | | AYMELSRLRSDDTAVYYCARAGICSG | | SKSGTSASLAITGLQAEDEADY |
| | | TTCYGYGSDYWGQGTLVTVSS | | YCQSYDSSLSGYVFGTGTQLTV |
| | | | | L |
| 11B05 | 4095 | QVQLVQSGAEVKKPGASVKVSCKASG | 4253 | QSVLTQPPSVSGAPGQRVTISC |
| | | YTFTGYYIHWVRQAPGQGLEWMGWII | | TGSSSSIGAGYDVHWYQQLPGT |
| | | PNSGGTKYAQNFQGRVTMTRDTSIST | | APKLLIYGNSNRPSGVPDRFSG |
| | | AYMELSRLRSDDTAVYFCARAGYSSA | | SKSGTSASLAITGLQAEDEADY |
| | | WYDYWGQGTLVTVSS | | YCQSYDSSLSGWVFGGGTKLTV |
| | | | | L |
| 11A03 | 4096 | EVQLVQSGAEVRRPGASVRVSCKASG | 4254 | QSVLTQPPSVSGAPGQRVTISC |
| | | YTFTDYYMHWVRQAPGQGLEWVGWII | | TGSSSNIGAGYDVHWYQQLPGT |
| | | PNSGGTKYAQNFQGRVTLTRDTSIRT | | APKLLIYGNSNRPSGVPDRFSG |
| | | AHMDLTRLRSDDTAVYFCARAGYSSS | | SKSGTSASLAITGLQAENEADY |
| | | WYDNWGQGTTVTVSS | | YCQSYDSSLSGWVFGGGTKLTV |
| | | | | L |
| 11H08 | 4097 | EVQLVESGAEVKKPGASVKVSCKASG | 4255 | QAVLTQPPSVSGAPGQRVTISC |
| | | YTFTNYYIHWVRQAPGQGLEWMGWII | | TGSSSNIVAGYDVHWYQQLPGT |
| | | PNSGGTKYAQKFQGRVTMTRDTSIST | | APKLLIYGNTNRPSGVPDRFSG |
| | | AYMDLSRLRSDDTAIYYCARAGYSSS | | SKSGTSASLAITGLQAEDEADY |
| | | WYEYWGQGTLVTVSS | | YCQSYDSSLSGWVFGGGTKVTV |
| | | | | L |
| 10F01 | 4098 | EVQLVESGGGVVQPGRSLRLSCAASG | 4256 | QSVVTQPPSVSGAPGQRVTISC |
| | | FTFSGYDMHWVRQAPGKGLEWLAVIW | | TGSSSNIGAGYDVHWYQQLPGT |
| | | YDGSNKYYADSVKGRFTISRDNSKNT | | APKLLIYGNSNRPSGVPDRFSG |
| | | LYLQMNSLRAEDTAVYYCARAIAAAG | | SKSGTSASLAITGLQAEDEADY |
| | | FDYWGQGTTVTVSS | | YCQSYDSSLSGSVFGGGTQLII |
| | | | | L |
| 08C09 | 4099 | QVQLVESGAEVKKPGASVKVSCKASG | 4257 | QAVLTQPPSVSGAPGQRVTISC |
| | | YTFTGNYIHWLRQAPGQGLEWMGWIH | | TGSSSNIGAGYDVHWYQQLPGT |
| | | PYSGGTKYAQKFQGRVTMTRDTSIST | | APKLLIYGNSNRPSGVPDRFSG |
| | | AYMDLNRLRSDDTAVYYCARAPAGTP | | SKSGTSASLAITGLQAEDEADY |
| | | YFDYWGQGTTVTVSS | | YCQSYDSSLSGWVFGGGTQLTV |
| | | | | L |
| 10F08 | 4100 | EVQLVQSGAEVKKPGASVKVSCKASG | 4258 | QSVLTQPPSVSGAPGQRVTISC |
| | | YTFTGYYMHWVRQAPGQGLEWLGWII | | TGSSSNIGAGYDVHWYQQLPGT |
| | | PNSGGTKYAQKFQGRVTMARDTSLST | | APKLLIYGNSNRPSGVPDRFSG |
| | | AYMELTSLGSDDTAFYYCARASYSSS | | SKSGTSASLAITGLQAEDEADY |
| | | WYDYWGQGTLVTVSS | | YCQSYDSSLSGWVFGGGTKLTV |
| | | | | L |
| 11F06 | 4101 | EVQLVQSGAEVKKPGASVKVSCKASG | 4259 | QAVLTQPPSVSGAPGQRVTISC |
| | | YTFTGYYIHWVRQAPGQGLEWMGWII | | TGSSSNIGAGYDVHWYQQLPGT |
| | | PNSGGTKYAQKFQGRVTMTRDTSTST | | APKLLIYGNSNRPSGVPDRFSG |
| | | VYMEMSRLRSDDTAVYYCARAYINSA | | PKSGTSASLAITGLQAEDEADY |
| | | YFDYWGQGTLVTVSS | | YCQSYDSSLSGWVFGGGTKVTV |
| | | | | L |
| 05H05 | 4102 | EVQLVESGGGVVRPGGSLRLSCAASG | 4260 | DVVMTQSPLSSPVTLGQPASIS |
| | | FTFGDYGMSWVRQAPGKGPEWVSGIN | | CRSSQSLVSSVGNTYLNWFQQR |
| | | WNGDSKGYVDSVKGRFSISRDNAKNS | | PGQSPRRLIYKVSDRDSGVPDR |
| | | LYLEMNSLRAEDTALYYCARDDFYYY | | FSGSGSGTDFTLKISRVEAEDV |
| | | MDAWGRGTTVTVSS | | GVYYCMQGTHWPPTFGQGTKLE |
| | | | | IK |
| 06A09 | 4103 | EVQLVESGGGLVKPGGSLRLSCAASG | 4261 | AIQMTQSPSFLSASVGDRVTIT |
| | | FTFSSYSMNWVRQAPGKGLEWVSVIS | | CRASQGIGSYLAWYQQKPGKAP |
| | | GSGGGTFYADSVKGRFTVSRDNSKNT | | KLLIYAASTLQSGVPSRFSGSG |
| | | LYLQINSLRAEDTAVYYCARDDWNYE | | SGTEFTLTISSLQPEDFATYYC |
| | | AFDIWGQGTMVTVSS | | QQLNSYPLTFGGGTKLEIK |
| 01E06 | 4104 | QVQLVQSGAEVKKPGASVRVSCKASG | 4262 | AIRMTQSPSFLSASVGDRVTIT |
| | | YTFTNYGISWVRQAPGQGLEWMGWIS | | CRASQGISSYLAWYQQRPGKAP |
| | | AYNGNTNYAQKLQGRVTMTTDTSTST | | KLLIYAASTLQSGVPSRFSGSG |
| | | AFMELRSLRSDDTAVYYCARDDYYYD | | SGTEFTLTISSLQPEDFATYYC |
| | | GSGFDFWGQGTTVTVSS | | QQFHSYPFTFGPGTKVEIK |
| 02F06 | 4105 | QVTLKESGGGVVRPGGSLRLSCAASG | 4263 | DVVMTQSPLSLPVTLGQPASIS |
| | | FTFGDYGMSWVRQVPGKGLEWVSGIN | | CRSSQSLVYSYGNTYLNWFQQR |
| | | WNGDSTGYVDSVKGRFTISRDNAKNS | | PGQSPRRLIYKVSNRDSGVPDR |
| | | LYLQMNSLRAEDTALYYCARDDYYYY | | FSGSGSGTDFTLKISRVEAEDV |
| | | MDVWGKGTTVTVSS | | GVYYCMQGTYWPPTFGGGTKLE |
| | | | | IK |
| 04B04 | 4106 | QVQLVQSGAEAKKPGTSVTVSCKASG | 4264 | DIQLTQSPSFLSASVGDRVTIT |
| | | YTFTSYYMHWVRQAPGQGLEWVGVIS | | CRASQGISSYLAWYQQKPGKAP |
| | | PTGGGTHYAQKFQGRVTMTRDTSTST | | KLLIYAASSLQGGVPSRFSGSG |
| | | VSMELTSLRSEDTAVYYCARDEAGDD | | SGTDFTLTISSLQPEDSATYYC |
| | | AFDIWGQGTMVTVSS | | LQDYNYPLTFGGGTKLEIK |
| 04D11 | 4107 | EVQLVQSGAEAKKPGTSVTVSCKASG | 4265 | DIQLTQSPSFLSASVGDRVTIT |
| | | YTFTSYYMHWVRQAPGQGLEWVGVIS | | CRASQGISSYLAWYQQKPGKAP |
| | | PTGGGTHYAQKFQGRVTMTRDTSTST | | KLLIYAASTLQSGVPSRFSGSG |
| | | VSMELTSLRSEDTAVYYCARDEAGDD | | SGTEFTLTISSLQPEDFATYYC |
| | | AFDIWGQGTMVTVSS | | QQLNSYPITFGQGTRLEIK |
| 04D07 | 4108 | QVQLVQSGAEAKKPGTSVTVSCKASG | 4266 | AIQMTQSPSFLSASVGDRVTTT |
| | | YTFTSYYMHWVRQAPGQGLEWVGVIS | | CRASQGISSYLAWYQQKPGKAP |
| | | PTGGGTHYAQKFQGRVTMTRDTSTST | | KLLIYAASTLQSGVPSRFSGSG |
| | | VSMELSSLRSEDTAVYYCARDEAGDG | | SGTEFTLTISSLQPEDFATYYC |
| | | AFDIWGQGTMVTVSS | | QQLNSYPLTFGGGTKLEIK |
| 06C09 | 4109 | EVQLVESGPGLVKPSETLSLTCTVSG | 4267 | EIVMTQSPATLSLSPGERATLS |
| | | GSIRSTNYYWGWIRQPPGKGLEWIGS | | CRASQSVSSYLAWYQQKPGQAP |
| | | IYYSGITYYNPSLKSRVTISVDTSKN | | RLLIYDASNRATGIPARFSGSG |
| | | QFSLKLSSVTAADTAVYYCARDGWDA | | SGTDFTLTISSLEPEDFAVYYC |
| | | FDIWGQGTMVTVSS | | QQRSNWPYTFGQGTKLEIK |
| 08F05 | 4110 | QVQLQQSGPGLVKPSQTLSLTCAISG | 4268 | QAGLTQPPSVSVSPGQTASITC |
| | | DSVSSNSATWNWIRQSPSRGLEWLGR | | SGDKLGSKYAFWYQQKPGQSPV |
| | | TYYRSKWYNDYAVSVKSRITINADTY | | VVIYQDNKRPSGIPERFSGSNS |
| | | KNQFSLHLNSVTPEDTAVYYCARDGW | | GNTATLTISGTQAIDEADYYCQ |
| | | NDDFDYWGQGTTVTVSS | | AWDGSSAVFGGGTKVTVL |
| 10F04 | 4111 | QVQLQQSGPGLVKPSQTLSLTCAISG | 4269 | SYELTQPLSVSVALGQTARITC |
| | | DSVSSHSGAWNWLRQSPSRGLEWLGR | | GGNNIGSENVYWYQQKPGQAPV |
| | | TYYRSKWYNDYAVSVKSRITINPDTS | | VVIYRDSNRPSGIPERFSGSNS |
| | | KNQFSLQLNSVTPEDTAVYYCARDGW | | GNTATLTISRAQAGDEADYYCQ |
| | | TDAFDIWGQGTMVTVSS | | VWDSSTVVFGGGTKLTVL |
| 09F01 | 4112 | QVQLVESGGGLVKPGGSLRLSCAASG | 4270 | SYELTQPPSVSVSPGQTARITC |
| | | FTFSNAWMSWVRQAPGKGLEWVGRIK | | SGDALPKKYAYWYQQKSGQAPV |
| | | SKTDGGTTDYAAPVKGRFTISRDDSK | | LVIYEDSKRPSGIPERFSGSSS |
| | | NTLYLQMNSLKTEDTAVYYCTTDHPY | | GTMATLTISGAQVEDEADYYCY |
| | | YWGQGTLVTVSS | | STDSSGNHYVFGTGTKLTVL |
| 05H09 | 4113 | QVQLQQSGPGLVKPSQTLSLTCDISG | 4271 | DIVMTQTPLSLPVTLGQPASIS |
| | | DSVSSNIAAWNWIRQSPSRGLEWLGR | | CRSSQSLVYSDGNTYLNWFQQR |
| | | TYYRSKWYNEYAVSVKGRMTITPDTS | | PGQSPRRLIYKVSNRDSGVPDR |
| | | KNHFSLQLNSVTPEDTAVYYCARDLH | | FSGSGSGTDFTLKISRVEAEDV |
| | | SSTLDYWGQGTLVTVSS | | GVYYCMQGTHWPRTFGQGTKVD |
| | | | | IK |
| 06C12 | 4114 | QVQLQQSGPGLVKPSQTLSLTCDISG | 4272 | DVVMTQSPLSLPVTLGQPASIS |
| | | DSVSSNSTAWNWIRQSPSRGLEWLGR | | CRSSQSLVYSDGNTYLNWFQQR |
| | | TYYWSKWYNEYAVSVKGRIIITPDTS | | PGQSPRRLIYKVSNRDSGVPDR |
| | | KNHFSLQLNSVTSEDTAVYYCARDLH | | FSGSGSGTDFTLKISRVEAEDV |
| | | SSTLDYWGQGTTVTVSS | | GVYYCMQGTHWPRTFGQGTKLE |
| | | | | IK |
| 07C09 | 4115 | QVQLVESGGGLVQPGGSLRLSCAASG | 4273 | QSALTQPASVSGSPGQSITISC |
| | | FTFTSYAMTWVRQVPGKGLEWVSAIS | | TGTSSDVGGYNYVSWYQQHPGK |
| | | GSGGQTFSADSVKGRFTISRDNSKNT | | APKLMIYEVSNRPSGVSNRFSG |
| | | LYLQMNSLRAEDTAVYYCAKDLKWEI | | SKSGNTASLTISGLQAEDEADY |
| | | LPDAFDIWGQGTMVTVSS | | YCSSYTSSSTLVVFGGGTQVTV |
| | | | | L |
| 07E03 | 4116 | EVQLVQSGGDLVQPGGSLRLSCATSG | 4274 | QSALTQPASVSGSPGQSITISC |
| | | FTFSNYAMTWVRQAPGKGLEWVSAIT | | TGTSSDVGGYNYVSWYQQHPGK |
| | | GSGGNTFSADSVKGRFTISRDNSKNT | | APKLMIYEVSNRPSGVSNRFSG |
| | | LYLQMNSLRAEDTAVYYCAKDLKWEL | | SKSGNTASLTISGLQAEDEADY |
| | | LPDAFDIWGHGTMVTVSS | | YCSSYTSSSTLVFGGGTKLTVL |
| 02H08 | 4117 | EVQLVESGGGLVQPGGSLRLSCAASG | 4275 | DIVMTQTPLSLPVTPGEPASIS |
| | | FTFSGYDVNWVRQAPGKGLEWISGIS | | CRSSQSLLHSNGYNYLDWYLQR |
| | | SSGESTYYADSVKGRFTISRDNSKRT | | PGQSPQLLIYLGSNRASGVPDR |
| | | LYLDMSSLRAEDTAFYYCARDMGFNW | | FSGSASGTDFTLKISRVEAEDV |
| | | NYGAFNMWGQGTMVTVSS | | GVYYCMQGLQTPWTFGQGTKVE |
| | | | | IK |
| 06D02 | 4118 | QVQLVQSGAEVKKPGASVKVSCKASG | 4276 | DIVMTQTPLSLPVTLGQPASIS |
| | | YTFSGYYMHWVRQAPGQGLEWMGWIN | | CRSSRSLVYSDGNTYLNWFQQR |
| | | PNSGGTNYAQKFQGRVTMTRDTSINT | | PGQSPRRLIYKVSNRDSGVPDR |
| | | AYMELSRLRSDDTAVYYCARDNWNSF | | FSGSGSGRDFTLKISRVEAEDV |
| | | DFWGQGTLVTVSS | | GVYYCMQGTNWPPYTFGQGTKL |
| | | | | EIK |
| 07E04 | 4119 | EVQLVESGGGLVQPGGSLRLSCAASG | 4277 | QAGLTQPPSASGTPGQRVTISC |
| | | FTFSDYYMSWIRQAPGKGLEWVSYIS | | SGSSSNIGRNPVYWYQQLPGTA |
| | | GPGSTIYYADSVKGRETTSRDNAKNS | | PKLLIYDNNQRPSGVPDRFSGS |
| | | LYLQMNSLKAEDTAVYYCARDNWNYG | | KSGTSASLAISGLQSEDEGDYY |
| | | FDYWGQGTLVTVSS | | CAAWDDSLEVFGGGTQLTVL |
| 02C03 | 4120 | QVQLQESGPGLVKPSGTLSLTCAVSG | 4278 | DIVLTQSPSSVSASVGDRVTIT |
| | | GSIISNNWWSWVRQSPGKGLEWIGEI | | CRASQGIAGWLAWYQQKPGKAP |
| | | YHNGTTSYNPSLESRVTISIDKSKNQ | | KLLIYSASSLQSGVPSRFSGSG |
| | | LSLRLRSVTAADTAVYYCARDQEGLT | | SGTDFTLTISSLQPEDFATYYC |
| | | GCHDYWGQGTLVTVSS | | QQANSFPRTFGQGTKLEIK |
| 08F02 | 4121 | QVQLVQSGSELKKPGASVKVSCKASG | 4279 | QAVLTQPPSVSGAPGQRVTISC |
| | | YTFTSYAMNWVRQAPGQGLEWMGWIN | | TGSSSNIGAGYDVHWYQQLPGT |
| | | TNTGNPTYAQGFTGRFVFSLDTSVST | | APKLLIYGNSNRPSGVPDRFSG |
| | | AYLQISSLKAEDTAVYYCARDRNYYG | | SKSGTSASLAITGLQAEDEADY |
| | | SGSYYGDYWGQGTTVTVSS | | YCQSYDSSLSGSMVFGGGTKVT |
| | | | | VL |
| 06F01 | 4122 | QVQLVQSGAEVKKPGSSVKVSCKASG | 4280 | DIVMTQSPSTLSASVGDRVTIT |
| | | GTLSRYAVNWVRQAPGQGLEWMGGII | | CRASQSISNWLAWYQQKPGKAP |
| | | PIFGTVNYAQKFQGRVTITADESTST | | KLLIYKASSLQSGVPSRFSGSG |
| | | AYMELSSLRSEDTALYYCASDRSGTT | | SGTEFTLTISSLQPDDFATYYC |
| | | NWFFDLWDRGTLVTVSS | | QQYKSFITFGQGTRLEIK |
| 10A04 | 4123 | EVQLVESGGGLIQPGGSLRLSCAASG | 4281 | QAGLTQPPSVSVSPGQTASITC |
| | | FTFSNYAMNWVRQAPGKGLEWVSAIL | | SGDKLGDKYVSWYQQTPGQSPV |
| | | GSGGNTYYADSVKGRFTISRDNSKNT | | LVIYQDSERPSGIPERFSGSNS |
| | | LYLQMNSLRAEDTAVYYCAKDRSNWN | | GNTATLTISGTQAMDEADYYCQ |
| | | FGDWFDPWGQGTLVTVSS | | AWDSSTVVFGGGTKLTVL |
| 07C08 | 4124 | QVQLVQSGSELKKPGASVRVSCKASG | 4282 | QAGLTQPPSASGTPGQRVTISC |
| | | YTFTNYAMNWVRQAPGQGLEWMGWIN | | SGSSSNIGSNTVNWYQQLPGTA |
| | | TNTGNPTYAQGFTGRFVFSLDTSVST | | PKLLIYSNNQRPSGVPDRFSGS |
| | | AYLQISSLKAEDTAVYYCARDRYYYG | | KSGTSASLAISGLQSEDEADYY |
| | | SGTYFFDFWGQGTTVTVSS | | CAAWDDSLNGVVFGGGTKVTVL |
| 04B08 | 4125 | QVQLVQSGAEVKKPGASVKVSCKASG | 4283 | DIQMTQSPSSLSASVGDRVTIT |
| | | YTFTNYGISWVRQAPGQGLEWMGWIS | | CRASQSISSYLNWYQQKPGKAP |
| | | AYNGNTNYAQKLQGRVTMTTDTSTST | | KLLIYAASSLQSGVPSRFSGSG |
| | | AYMELRGLRSDDTAVYYCARDSRGWF | | SGTDFTLTISSLQPEDFATYYC |
| | | DPWGQGTLVTVSS | | QQSYSTPLTFGQGTRLEIK |
| 07D05 | 4126 | EVQLVQSGGGLVQPGGSLRLSCAASG | 4284 | QAGLTQPPSVSVAPGKTARITC |
| | | FTFSSYSMNWVRQAPGKGLEWVSYIS | | GGNNIGSKSVHWYQQKPGQAPV |
| | | SSSSTIYYADSVKGRFTISRDNAKNS | | LVIYYDSDRPSGIPERFSGSNS |
| | | LYLQMNSLRDEDTAVYYCARDVGGNY | | GNTATLTISRVEAGDEADYYCQ |
| | | YYNMDVWGKGTTVTVSS | | VWDSSSDHVVFGGGTQLTVL |
| 03C12 | 4127 | EVQLVQSGAEVMKPGASVKVSCKASG | 4285 | DIQMTQSPSSLSASVGDRVTIT |
| | | YTFTGYYMNWVRQAPGQGLEWMGLIS | | CRASQSISSYLNWYQQKPGKAP |
| | | PNSGDTYYSHKFQGRVTMTRDTSIST | | KLLVYAASSLQSGVPSRFSGSG |
| | | AYMELSRLRSDDTAVYYCARDYGDYD | | SGTDFTLTISSLQPEDFATYYC |
| | | GMDVWGKGTTVTVSS | | QQSYSTPITFGQGTRLEIK |
| 07G09 | 4128 | QVQLVQSGAEVKKPGASVKVSCKASG | 4286 | SYVLTQPPSASGTPGQRVTISC |
| | | YTFTGYYMHWVRQAPGQGLEWMGWIN | | SGSSSNIGSNTVNWYQQLPGTA |
| | | PNSGDTNYEQKFQGRVTMTRDTSIST | | PKLLIYSNNQRPSGVPDRFSGS |
| | | AYMELSSLRSDDTAVYYCAREDIAAV | | KSGTSASLAISGLQSEDEADYY |
| | | GENYYYYYMDVWGKGTTVTVSS | | CAAWDDSLNGYVFGTGTQLTVL |
| 07H01 | 4129 | QVQLVQSGAEVKKPGASVKVSCKASG | 4287 | QSALTQPPSASGTPGQRVTISC |
| | | YTFTGYYMHWVRQAPGQGLEWMGWIN | | SGSSSNIGSNTVNWYQQLPGTA |
| | | PNSGDTNYEQKFQGRVTMTRDTSIST | | PKLLIYSNNQRPSGVPDRFSGS |
| | | AYMELSRLRSDDTAVYYCAREDIAAV | | KSGTSASLAISGLQSEDEADYY |
| | | GENYYYYYMDVWGKGTTVTVSS | | CAAWDDSLNGYVFGTGTQVTVL |
| 07B01 | 4130 | EVQLVQSGAEVKKPGASVKVSCKPSG | 4288 | QAVLTQPRSVSGSPGQSVTISC |
| | | YTFSGYYLHWVRQAPGQGLEWVGWID | | TGTSSDVGGYNYVSWYQQHPGK |
| | | PNSGGTNSAQIFQGRVTMTRDTSIST | | APKLMIYDVSKRPSGVSNRFSG |
| | | AYMELSGLRSDDTAVYYCAREDTWNF | | SKSGNTASLTISGLQAEDEADY |
| | | VPYFDYWGQGTLVTVSS | | YCSSYTSSSTLVFGGGTKLTVL |
| 07A01 | 4131 | QMQLVQSGAEVKKPGASVKVSCKASG | 4289 | QSVLTQPPSVSGAPGQRVTISC |
| | | YTFTGSYIHWVRQAPGQGLEWMGWIN | | TGSSSNIGAGYDVHWYQQLPGT |
| | | PNSGATNYAQRFQGRVTMTRDSSIST | | APKLLIYGNSNRPSGVPDRFSG |
| | | AYMELSSLRSADTAVYYCAKEGIAPV | | SKSGTSASLAITGLQAEDEADY |
| | | DTGYYYYMDVWGKGTTVTVSS | | YCQSYDSSLSGWVFGGGTQLTV |
| | | | | L |
| 09F09 | 4132 | QVQLQESGPGLVKPSGTLSLTCAVSG | 4290 | SYVLTQPPSVSVSPGQTARITC |
| | | GSISSTNWWSWVRQPPGKGLEWIGAI | | SGDALPKKYAYWYQQKSGQAPV |
| | | YQTGNTYYNPSLKSRVTMSVDKSTNQ | | LVIYEDSKRPSGIPERFSGSSS |
| | | FSLKLNSVTAADTAVYYCAREGLYFG | | GTMATLTISGAQVEDEADYYCY |
| | | DLLSYAFDIWGQGTMVTVSS | | STDSSGNHRVFGGGTKVTVL |
| 07C01 | 4133 | QVQLVESGAEVKKPGASVKVSCKASG | 4291 | QSVLTQPPSVSGAPGQRVTISC |
| | | YTFTGNYIHWVRQAPGQGLEWMGWIN | | TGSSSNIGSGYDVHWYQQLPGT |
| | | PNSGHTNYAQNFQGRVTMTRDTSIST | | APKLLIYGNSNRPSGVPARFSG |
| | | AYMELNRLRSDDTAVYYCAKEGVAAA | | SKSGTSASLAITGLQAEDEADY |
| | | GTGFFYYMDVWGKGTLVTVSS | | YCQSYDSSLSGWVFGGGTKVTV |
| | | | | L |
| 06G01 | 4134 | EVQLVQSGGGLVQPGGSLRLSCEVSG | 4292 | AIQMTQSPSSLSPSVGDRVTIT |
| | | FTFSWYWMHWVRQVPGEGLVWVSRIK | | CRASQGIRNDLGWYQQKPGKAP |
| | | SDGSNTRYADSVKGRFTISRDNAKNT | | KLLIYAASTLQSGVPSRFSGSG |
| | | LYLQMSSLRAEDTAVYYCAREGWGIN | | SGTEFTLTISSLQPEDFATYYC |
| | | GLGGAFDIWGQGTMVTVSS | | QQLNSYPITFGQGTRLEIK |
| 06A01 | 4135 | QVQLVESGGGLVQPGGSLRLSCAASG | 4293 | AIRMTQSPSFLSASVGDRVTIT |
| | | FSFSWHWMHWVRQAPGKGLEWVSRIK | | CRASQGISSYLAWYQQKPGKAP |
| | | SDGSNTKYADSVKGRFTISRDNAKNT | | NLLLYAASTLQSGVPSRFSGSG |
| | | MYLQMNSLRDEDTAVYYCAREGWGIS | | SGTDFTLTISSLQPEDFATYYC |
| | | GLGGAFDIWGQGTMVTVSS | | QQLNSYPFTFGQGTKVDIK |
| 06B06 | 4136 | QVQLVESGGGLVQPGRSLRLSCAASG | 4294 | DIQMTQSPSFLSASVGDRVTIT |
| | | FTFSWFWLHWVRQAPGKGLVWVSRIK | | CRASQGISSYLAWYQQKPGKAP |
| | | SDGSSTKYADSVKGRFTISRDNAKNT | | KLLIYAASTLQSGVPSRFSGSG |
| | | LYLQMNSLRDEDTAVYYCAREGWGIT | | SGTEFTLTISSLQPEDFATYYC |
| | | GQGGALDIWGQGTMVTVSS | | QQLNSYPHTFGQGTKLEIK |
| 06A02 | 4137 | EVQLVESGGGLVQPGGSLRLSCEASE | 4295 | AIRMTQSPSSLSASIGDRVTIT |
| | | FTFSYYWLHWVRQAPGKGLVWVSRIN | | CRASQGIRNDLGWYQQKPGKAP |
| | | SDGSNTKYADSVKGRFTISRDNAKNT | | KRLIYAASSLQSGVPSRFSGSG |
| | | LYLQMNSLRAEDTAVYYCAREGWGMT | | SGTEFTLTISSLQPEDFATYYC |
| | | GLGGAFDIWGQGTMVTVSS | | LQLNSYPLTFGGGTKVDIK |
| 06F12 | 4138 | EVQLVQSGGGLVQPGGSLRLSCAASG | 4296 | EIVMTQSPATLSLSPGETVTLS |
| | | FTFSWYWMHWVRQVPGKGLVWVSRIK | | CRASQSVSRYLAWYQQKPGQPP |
| | | SDGGSPKYADSVKGRFTISRDNARNT | | RLLIYDASNRATGIPARFSGSG |
| | | LYLQMNSLGAEDTAVYYCTREGWGMT | | SGTDFTLTISSLEPEDFAVYYC |
| | | GLGGAFDIWGQGTMVTVSS | | QQRSNWPPITFGQGTKLEIK |
| 05D12 | 4139 | QLQLQESGGGLVKPGGSLRLSCAASG | 4297 | DVVMTQSPLSLPVTLGQPASIS |
| | | FTFTNYTMNWVRQAPGKGLEWVSSIS | | CRSSQSLVYSDGNTYLNWFQQR |
| | | SSGTYIYYADSVKGRFTISRDNAKNS | | PGQSPRRLIYKVSNRDSGVPDR |
| | | LHLQMNSLRAEDSAVYYCSREGYYDF | | FSGSGSGTDFTLKISRVEAEDV |
| | | LTGYISDAFDIWGQGTMVTVSS | | GVYFCMQGTHWPFTFGPGTKVE |
| | | | | IK |
| 05D07 | 4140 | QVQLVQSGGGLVKPGGSLRLSCAASG | 4298 | DVVMTQSPLSLPVTLGQPASIS |
| | | FTFSRYRMNWVRQAPGKGLEWVSSIS | | CRSSQSLVYSDGNTYLNWFQQR |
| | | SSTSYIFYADSVKGRFTISRDNAKNS | | PGQSPRRLIYKVSNRDSGVPDR |
| | | LYLHMNSLRAEDTAVYYCAREGYYNN | | FSGSGSGTDFTLKISRVEAEDV |
| | | SGYYFPDAFDIWGQGTMVTVSS | | GVYYCMQGTHWPFTFGPGTKVE |
| | | | | IK |
| 02F04 | 4141 | QVQLVQSGAEVKKPGASVKVSCKASG | 4299 | EIVMTQSPATLSLSPGERATLS |
| | | YTFTDYYVYWVRQAPGQGLEWLGWIN | | CRASQSVSSNLAWYQQKPGQAP |
| | | PDSGGTNYEQKFQGRVTMTRDTSIST | | RLLIYGASTRATGIPARFSGSG |
| | | AYMELSRLRSDDTAVYYCTREKGLRY | | SGTEFTLTISSLEPEDFAVYYC |
| | | SGYDRGYFDYWGQGTLVTVSS | | QQRSNWPLTFGGGTKLEIK |
| 02A07 | 4142 | EVQLVESGAEVRKPGASVKVSCKASG | 4300 | EIVMTQSPATLSLSPGERATLS |
| | | YTFTDYYIHWVRQAPGQGLEWMGWIN | | CRASQSVHKYLAWYQQKPGQAP |
| | | PNSGGTHYAQKFQGRVTLTRDTSIST | | RLLIYDASSRATGIPARFSGSG |
| | | AYMELSRLTSDDTAVYYCAKEMGLRY | | SGTDFTLTISSLEPEDFAVYYC |
| | | SGYDRGYFDYWGQGTLVTVSS | | QQRSNWPLTFGGGTKVEIK |
| 07C02 | 4143 | EVQLVESGPGLVKPSGTLSLTCAVSG | 4301 | QSVLTQPPSVSGAPGQRVTISC |
| | | GSISSNNWWSWVRQPPGKGLEWIGEI | | TGSSSNIGAGYDVHWYQQLPGT |
| | | YHSGSTSYNPSLKSRVTMSVDQSKNQ | | APKLLIYGNSNRPSGVPDRFSG |
| | | FSLQLTSVTAADTAMYYCARERGYDS | | SKSGTSASLAITGLQAEDEADY |
| | | EYYFYYYMDVWGKGTTVTVSS | | YCQSYDSSLSGYVFGTGTKLTV |
| | | | | L |
| 07D03 | 4144 | QVQLQESGPGLVKPSGTLSLTCAVSG | 4302 | QSVLTQPPSVSGAPGQRVTISC |
| | | GSINSSNWWSWVRQSPGKGLEWIGEI | | TGSSSNIGAGYDVHWYQQLPGT |
| | | YHSGSTSYNPSLKSRVTISVDKSKNQ | | APKLLIYGNSNRPSGVPDRFSG |
| | | FSLKLSSVTVADTAVYYCARERGYSS | | SKSGTSASLAITGLQAEDEADY |
| | | PHYYFYYYMDVWGKGTTVTVSS | | YCQSYDSSLSGYVFGTGTKLTV |
| | | | | L |
| 07G08 | 4145 | QVQLVESGGGLVKPGGSLRLSCAASG | 4303 | QAGLTQPPSVSVSPGQTASITC |
| | | FTFSYSTMTWVRQAPGKGLEWVSSIG | | SGDKLGNKYASWYQQKPGQSPV |
| | | GIGGYTYYADSVKGRFTISRDNSTNT | | LVIYQDRKRPSGIPERFSGSNS |
| | | MYLQMNSLRDEDTAVYYCAKERVAAN | | GNTATLTISGTQAMDEADYYCQ |
| | | YFDYWGQGTTVTVSS | | AWDSSTYVIFGGGTKVTVL |
| 07G01 | 4146 | QVQLVESGPGLVKPSGTLSLTCAVSG | 4304 | QSVVTQPPSVSGAPGQRVTISC |
| | | GSISSSNWWSWVRQTPGKGLEWIGEI | | TGSSSNIGAGYDVHWYQQLPGT |
| | | YHSGSTSYNPSLKSRVTMSVDKSNNQ | | APKLLIYGNGNRPSGVPDRFSG |
| | | FSLKLNSVTAADTAVYYCARFSGYDD | | SKSGTSASLAITGLQAEDEADY |
| | | DYYFYYYMDVWGKGTTVTVSS | | YCQSYDSSLSGYVFGTGTKLTV |
| | | | | L |
| 04B07 | 4147 | EVQLVQSGAEVKKPGASVRVSCKASG | 4305 | DIQMTQSPSFLSASVGDRVTIT |
| | | YTFTNYGISWVRQAPGQGLEWMGWIS | | CRASQGISSYLAWYQQKPGKAP |
| | | AYNGNTKYAQKLQGRVTMTTDTSTST | | KLLIYAASTLQSGVPSRFSGSG |
| | | AFMELRSLRSDDTAVYYCVRESSSWY | | SGTEFTLTISSLQPEDFATYYC |
| | | GPLFAIWGQGTMVTVSS | | QQLNSYPLTFGGGTKLEIK |
| 04C03 | 4148 | QVQLQESGPGLVKPSGTLSLTCAVSG | 4306 | DIQLTQSPSSLSASVGDRVTIT |
| | | GSISSSDLWSWVRQPPGKGLEWIGEI | | CRASQSISSYLNWYQQKPGKAP |
| | | YHSGTTNSNPSLKSRVTISVDKSKNQ | | KLLIYAASSLQSGVPSRFSGSG |
| | | FSLKLSSVTAADTAVYYCAREVYSSS | | SGTDFTLTISSLQPEDFATYYC |
| | | SGGYWYFDLWGRGTLVTVSS | | QQSYSTPFTFGPGTKVDIK |
| 05D03 | 4149 | QVQLQESGPGLVKPSGTLSLTCAVSG | 4307 | DIQLTQSPSSLSASVGDRVTIT |
| | | GSISSNNWWSWVRQSPGKGLEWIGEI | | CRAGQSISSYLNWYQQKPGKAP |
| | | YHTGSTNYNPSLKSRVSISVDKSKNQ | | KVLIYAASSLQSGVPSRFSGSG |
| | | LSLKLTSVTAADTAVYYCAGFVVAPD | | SGTDFTLTISSLQPEDFATYYC |
| | | PIGYFDPWGQGTLVTVSS | | QQSYSTPFTFGPGTKLEIK |
| 05G09 | 4150 | QLQLQESGPGLVKPSGTLSLTCAVSG | 4308 | DIQLTQSPSSLSASLGDRVTIT |
| | | GSISSSNWWSWVRQSPGKGLEWIGEI | | CRASQSISSYLNWYQQKPGKAP |
| | | YHSGSTNYNPSLKSRVTISVDKSKNQ | | KLLIFAASSLQSGVPSRFSGSG |
| | | FSLKLSSVTAADTAVYYCAGFVVAPV | | SGTDETLTISSLQPEDFATYFC |
| | | PIGYFDPWGQGTLVTVSS | | QQSYSTPFTFGPGTKVEIK |
| 02F02 | 4151 | QVQLQESGPGLVKPSGTLSLTCAVSG | 4309 | DIQLTQSPSSLSASVGDRVTIT |
| | | GSISSSDWWSWVRQSPGKGLEWIGEV | | CRASQSISSYLNWYQQKPGKAP |
| | | YHSGSTNYNPSLRSRVTISVDKSKNQ | | ELLIYAASSLQGGVPSRFSGSG |
| | | FSLKLTSVTAADTAVYYCARFVVEPD | | SGTDFTLTISSLQPEDFATYYC |
| | | LIGWFDPWGQGTTVTVSS | | QQSYSTPLTFGGGTKVEIK |
| 12E03 | 4152 | QVQLQESGPGLVKPSGTLSLTCAVSG | 4310 | QAGLTQPPSVSVSPGQTASITC |
| | | GSISSSNWWSWVRQPPGKGLEWIGEI | | SGDKLGNKYASWYQQKPGQSPV |
| | | YHSGSTNYNPSLKSRVTISVDKSENQ | | LVIYQDSKRPSGIPERFSGSNS |
| | | FSLKLSSVTAADTAVYYCARGAAVDG | | GNTATLTISGTQAMDEADYYCQ |
| | | AGFDYWGQGTLVTVSS | | AWDSSTAVVFGGGTQLTVL |
| 10F02 | 4153 | QVQLQESGPGLVKPSGTLSLTCAVSG | 4311 | QAGLTQPPSVSVSPGQTASITC |
| | | GSISSSNWWTWVRQPPGKGLEWIGEI | | SGDKLGDKYVSWYQQKPGQSPV |
| | | YHSGSSNYNPSLKSRVTMSVDKSKNQ | | MVIYQDRKRPSGIPERFSGSNS |
| | | FSLKLSSVTAADTAVYYCARGATGDG | | GNTATLTISGTQAMDEADYYCQ |
| | | AGFDYWGQGTLVTVSS | | AWDSSTAVVFGGGTKLTVL |
| 10A03 | 4154 | QLQLQESDPGLVKPSGTLSLTCAVSG | 4312 | QAGLTQPPSVSVSPGQTASITC |
| | | GSISSSNWWSWVRQPPGKGLEWIGEI | | SGDKLGYKYASWYQQKPGQSPV |
| | | YHSGSTNYNPSLKSRVTISVDKSENQ | | LVIYQDRKRPSGIPERFSGSNS |
| | | FSLKLSSVTAADTAVYYCARGATVDG | | GNTATLTISGAQSMDEADYYCQ |
| | | AGFDYWGQGTLVTVSS | | AWDSSTAVVFGGGTKVTVL |
| 05F11 | 4155 | QVQLQQSGPGLVKPSQTLSLTCAISG | 4313 | DIVMTQSPSSLSASVGDRVTIT |
| | | DSVSSNSAAWNWIRQSPSRGLEWLGR | | CRASQSISSYLNWYQQKPGKAP |
| | | TYFRSEWYTDYAVSVKSRITINPDTS | | KLLIYAASSLQSGVPSRFSGSG |
| | | KNQFSLQMNSVTPEDTSMYYCARGDI | | SGTDFTLTISSLQPEDFATYYC |
| | | AAAGYYFDYWGQGTLVTVSS | | QQSYSTPLTFGGGTKVEIK |
| 04E01 | 4156 | QVQLQQSGPGLVKPSQTLSLTCAISG | 4314 | DIQLTQSPSSLSASVGDRVTIT |
| | | DSVSTNSAAWNWIRQSPSRGLEWLGR | | CRASQSISSYLNWYQQKPGKAP |
| | | TYYRSRWYNDYAVSVKSRLTINPDTS | | KLLIYAASSLQSGVPSRFSGSG |
| | | KNQIYLQLNSVTPEDTAVYYCARGDI | | SGPDFTLTISSLQPEDFATYYC |
| | | GAAGYYFDYWGQGTLVTVSS | | QQSYSPPLTFGGGTKLEIK |
| 04A08 | 4157 | QVQLQQSGPGLVKPSQTLSLTCAISG | 4315 | DIVMTQSPSSLSASVGDRVTIT |
| | | DSVSSNSAAWNWIRQSPSGALEWLGR | | CRASQSIGSYLNWYQQKPGKAP |
| | | TYYRSKWYNDYGVSVTGRIIINLDTS | | KFLIYGASGLQSGVPSRFSGSG |
| | | KNQFSLQLNSVTPEDTAVYYCARGDI | | SGTDFTLTISSLQTEDFATYYC |
| | | GTAGFYFDYWGQGTLVTVSS | | QQSFSTPFTFGPGTKVDIK |
| 04E03 | 4158 | QVQLQQSGPGLVKPSQTLSLTCAVSG | 4316 | DIQLTQSPSSLSASVGDRVTIT |
| | | DSVSTNGASWNWVRQSPSRGLEWLGR | | CRASRNIGTYLNWYQQKPGKAP |
| | | TYYRSDWYNDYTGSVIGRITINPDTS | | NLLIYAASSLQSGVPSRFGGSG |
| | | RNQFSLHLNSVTPEDTAVYYCARGDI | | SGTDFTLSISSLQPEDFATFYC |
| | | GTAGYYFDSWGQGTLVTVSS | | QQSFSTPYTFGQGTKVDIK |
| 04H07 | 4159 | QVQLQQSGPGLVKPSQTLSLTCAISG | 4317 | AIQMTQSPSSLSASVGDRVTIT |
| | | DSVSTNSASWNWIRQSPSRGLEWLGR | | CRASQSISNYLNWYQQKPGKAP |
| | | TYYRSNWYTDYAVSVISRMTINLDTS | | KLLIYAASSLQSGVPSRFSGSA |
| | | KNQFSLQLNTVTPEDTAVYYCTRGDV | | SGTDFTLTISGLQPEDFATYYC |
| | | GSAGYFFDYWGQGTLVTVSS | | QQTYSTPLTFGGGTKLEIK |
| 08F01 | 4160 | QVQLVESGGGLVKPGGSLRLSCSASG | 4318 | QSALTQPASVSGSPGQSITISC |
| | | FTFSTSLMNWVRQAPGKGLEWVSSIT | | TGTSSDVGGYNYVSWYQQHPGK |
| | | SGGTYISYTDSVKGRFTISRDNAKNS | | APKLMIYEVSNRPSGVSNRFSG |
| | | LFLHMDSLRAEDTAVYYCARGDWNYP | | SKSGNTASLTISGLQAEDEADY |
| | | PYYYYMDVWGKGTTVTVSS | | YCSSYTSSSTVVFGGGTKVTVL |
| 04G03 | 4161 | RVQLQQSGLGLVKPSQTLSITCDISG | 4319 | DIQLTQSPSSLSASVGDRVTIT |
| | | DSVSRNSVAWNWIRQSPSRGLEWLGR | | CRASQSISSYLNWYQQKPGKAP |
| | | TDYRSMWSGDYVESVQGRIIINLDTS | | KLLIYAASSLQSGVPSRFSGSG |
| | | KNQFSLHLSSVTPEDTAVYYCARGEA | | SGTDFTLTISSLQPEDFATYYC |
| | | GSAGFYFDYWGQGTLVTVSS | | QQSYSALTFGGGTKLEIK |
| 02A09 | 4162 | QVQLQQSGPGLVKPSQTLSLTCAISG | 4320 | DIVMTQTPLSLPVTLGQPASIS |
| | | DSVSRNSSAWNWIRQSPSRGLEWLGR | | CRSSQSLVYSDGNTYLNWFQQR |
| | | TYYWSKWYYEYAISVKGRMTINPDTS | | PGQSPRRLIYEVSNRDSGVPDR |
| | | KNQFSLQLHSVTPEDSAVYYCVRGEA | | FSGSGSGTDFTLKISRVEAEDV |
| | | GTLDYWGQGTLVTVSS | | GVYYCMQGTHWPYTFGQGTKLE |
| | | | | IK |
| 04F08 | 4163 | QVQLQQSGPGRLKPSQTLSLTCAISG | 4321 | DIQMTQSPSSLSASVGDRVTIS |
| | | DSVSSNSAAWNWIRQSPSRGLEWLGR | | CRASQTIISYLNWYQQKPGNAP |
| | | TYYRSNWFGDNAASVQGRITINPDTS | | KLLIYAASSLHSGVPSRFSGSG |
| | | KNQLSLHLHSLTPEDTAIYYCARGET | | SGTDFTLTISSLQPDDFATYYC |
| | | GTAGYYFDSWGQGTLVTVSS | | QQSYSTPITFGQGTRLEIK |
| 01E11 | 4164 | QVQLQQSGPGLVKPSQTLSLTCDISG | 4322 | DIQMTQSPSSLSASVGDRVTIT |
| | | DSVSSISASWNWIRQSPSRGLEWLGR | | CRASQSISSYLNWYQQKPGKAP |
| | | TYYRSKWYNDYGVSVKSRMTINPDTS | | ELLIYAASSLQGGVPSRFSGSG |
| | | KNQFSLQLNSVTPEDTAVYYCARGET | | SGTDFTLTISSLQPEDFATYYC |
| | | GTAGYYFDYWGQGTLVTVSS | | QQSYSTPLTFGGGTKLEIK |
| 04F09 | 4165 | QVQLQQSGPGLVKPSQTLSLTCAISG | 4323 | DIVLTQSPSSLSASVGDRVTIT |
| | | DSVSSNSAAWNWIRQSPSRGLEWLGR | | CRASQSISSYLNWYQQKPGKAP |
| | | TYYRSNWYGDYAVSVQGRITINPDTS | | KLLIYAASSLQSGVPSRFTGSG |
| | | KNQFSLQLNSLTPEDTAVYYCARGET | | SGTDFTLTISSLQPEDFATYFC |
| | | GTAGYYFDYWGQGTLVTVSS | | QQSYSPPLTFGGGTKVDIK |
| 04E05 | 4166 | QVQLQQSGPGLVKPSQTLSLTCAISG | 4324 | AIQMTQSPSSLSASVGDRVTIT |
| | | DSVSSKSASWNWIRQSPSRGLEWLGR | | CRASQSISSYLNWYQQKPGKAP |
| | | TYYRSEWYNDYALSVISRIIINPDTS | | KLLIYAASSLQSGVPSRFSGSG |
| | | KNQFSLQLNSVTPEDTAVYYCARGET | | SGTDFTLTISSLQPEDFATYYC |
| | | GTAGYYMDVWGKGTTVTVSS | | QQSYSTPLTFGGGTKVDIK |
| 04C02 | 4167 | QVQLQQSGPGLVKPSQTLSLTCAISG | 4325 | AIRMTQSPSSLSASVGDRVTIT |
| | | DSVSNNSAAWNWIRQSPSRGLEWLGR | | CRASQSISSYLNWYQQKPGKAP |
| | | TYYRSEWENDYGASVIGRIIINPDTS | | KLLIYAASSLQSGVPSRFSGSG |
| | | KNQFSLQLNSVTPEDTAIYYCARGEV | | SGTDFTLTISSLQPEDFAGYYC |
| | | GASGYYMDVWGKGTTVTVSS | | QQSYSTPLTFGGGTKVEIK |
| 01E02 | 4168 | QVQLQQSGPGLVKPSQTLSLTCDISG | 4326 | DIVMTQSPSSLSASVGDRVTIT |
| | | DSVSSNGATWNWIRQSPSRGLEWLGR | | CRASQSISSYLNWYQQKPGKAP |
| | | TYYRSQWYNDYGVSVLSRITINPDTS | | KLLIYAASSLQSGVPSRFSGSG |
| | | KNQFSLQLNSVTPEDTAVYYCARGGI | | SGTDFTLTISSLQPEDFATYYC |
| | | AGAGTLFDYWGQGTLVTVSS | | QQSYSTPLTFGGGTKVEIK |
| 01H04 | 4169 | QVQLQQSGPGLVKPSQTLSLTCDISG | 4327 | DIVMTQSPSSLSASVGDRVTIT |
| | | DSVSSNSATWNWIRQSPSRGLEWLGR | | CRASQSISSYLNWYQEKPGKAP |
| | | TYYRSKWYNDYSVSVTSRMTINPDTS | | KLLIYAASSLQTGVPSRFSGSG |
| | | KNQFSLQLNSVTPEDTAVYYCARGGI | | SGTDFTLTISSLQSEDFATYYC |
| | | ASAGTFFDYWGQGTTVTVSS | | QQSYSTPLTFGGGTKVDIK |
| 04C10 | 4170 | QVQLQQSGPGLVKPSQTLSLTCAISG | 4328 | DIVLTQSPSSLSASVGDRVTIT |
| | | DSVSNNSASWNWIRQSPSRGLEWLGR | | CRASQSISTYLNWYQQKPGKAP |
| | | TFYRSNWYNDYSVSVISQITINTDTS | | KLLIYAASSLQSGVPSGFSGSG |
| | | ANQFSLHLNSVTPEDTAVYFCARGGI | | SGTDFTLTISSLRPEDFATYYC |
| | | GAAGYYFDYWGQGTLVTVSS | | QQSYSTPITFGQGTRLEIK |
| 08E10 | 4171 | EVQLVQSGAEVKKPGASVKVSCKASG | 4329 | QSVLTQPPSVSGAPGQRVTISC |
| | | YTFTGYYMHWVRQAPGQGLEWMGWIN | | TGSSSNIGAGYDVHWYQQLPGT |
| | | PNSGGTNYAQKFQGRVTMTRDTSIST | | APKLLIYGNSNRPSGVPDRFSG |
| | | AYMELSRLRSDDTAVYFCARGGVCSG | | SKSGTSASLAITGLQAEDEADY |
| | | ITCYGYGSDYWGQGTLVTVSS | | YCQSYDSSLSGWVFGGGTKVTV |
| | | | | L |
| 07C03 | 4172 | EVQLVQSGAEVKKPGASVRVSCKASG | 4330 | QPVLTQPPSVSGAPGQRVTISC |
| | | YTFTGNYMHWVRQAPGQGLEWMGWIN | | TGSSSNIGAGYDVHWYQQLPGT |
| | | PNSGGTNYVQKFQGRVTMTRDTSIST | | APKLLIYGNSNRPSGVPDRFSG |
| | | AYMELSRLRSDDTAVYFCARGGVCSG | | SKSDTSASLAITGLQAEDEADY |
| | | TTCYGYGSDYWGQGTTVTVSS | | YCQSYDSSLSGWVFGGGTKVTV |
| | | | | L |
| 04H11 | 4173 | QVQLQQSGPGLVRPSQTLSLTCAISG | 4331 | AIQMTQSPSTLSASVGDRVTIT |
| | | DSVSHNSASWNWIRQSPSRGLEWLGR | | CRASQSISSYLNWYQQKPGKAP |
| | | TYYRSSWYNDYAVSVISRITINPDTS | | KLLIYAASSLQSGVPSRFSGSG |
| | | KNQFSLQLNSVTPEDTAVYYCARGGV | | SGTGFTLTISSLQPEDFATYYC |
| | | GPAGYYFDYWGQGTTVTVSS | | QQTYSTPLTFGGGTKVEIK |
| 03D07 | 4174 | EVQLVQSGAEVQKPGASVKVSCKASG | 4332 | DIQLTQSPSTLSASVGDRVTIT |
| | | ITFTGYYMHWVRQAPGQGLEWMGWIN | | CRASQSISTYLNWYQQKPGKAP |
| | | PKSDGTNYAQKFQGRVTMTRDTSIST | | KLLIYAASSLQSGVPSRFSGSG |
| | | SYMELSRLRSDDTAVYYCVRGGVYGS | | SGTDFTLTISSLQPEDFATYYC |
| | | RWPKAFDIWGQGTMVTVSS | | QQSYSTPLTFGGGTKLEIK |
| 05C08 | 4175 | QVQLVQSGGGLVQPGGSLRLSCAASG | 4333 | DIVMTQSPSSLSASVGDRVTIT |
| | | FTFRTYSMNWVRQAPGKGLEWISYIS | | CRASQGISNYLAWYQQKPGKVP |
| | | SSSSTIYYADSVKGRFTISRDNAKNS | | KLLIYAASTLQSGVPSRFSGSG |
| | | LYLQMNSLRDEDTAVYYCVRGGYDTS | | SGTDFTLTISSLQPEDFATYYC |
| | | GFAFDIWGQGTMVTVSS | | QQANSFPPTFGQGTKVDIK |
| 03C05 | 4176 | QVQLVQSGAEVKKPGASVKVSCKASG | 4334 | AIQMTQSPSSLSASIGDRVTIT |
| | | YTFTGYYMHWVRQAPGQGLEWMGWIN | | CRASQSISSYLNWYQQKPGKAP |
| | | PTSGATNYAQKFQGRITMTRDTSIST | | KILIYGASRLQSGVPSRFSGSG |
| | | VYMDLIRLRSDDTALYYCARGHLLLP | | SGTDFTLTISSLQPEDFATYYC |
| | | DAFDIWGHGTMVTVSS | | QQSYSTPITFGQGTRLEIK |
| 03H05 | 4177 | EVQLVESGAEVKKPGASVKVSCKASG | 4335 | DIVMTQSPSSLSASIGDRVTIT |
| | | YTFTGYYMHWVRQAPGQGLEWMGWIN | | CRASQSISSYLNWYQQKPGKAP |
| | | PTSGATNYAQKFQGRITMTRDTSIST | | KILIYGASRLQSGVPSRFSGSG |
| | | VYMDLIRLRSDDTALYYCARGHLLLP | | SGTDFTLTISSLQPEDFATYYC |
| | | DAFDIWGHGTMVTVSS | | QQSYSTPITFGQGTRLEIK |
| 01D10 | 4178 | QVQLVQSGSEVKSPGASVKVSCKASG | 4336 | AIRMTQSPSSVSASVGDRVTIT |
| | | YTLTGNYIHWVRQAPGQGLEWMGWIN | | CRASQNISSWLDWYQQKPGKAP |
| | | PNSGVTYSAQKLQGRVTMTRDTSIST | | TLLIYAASRLQSGVPSRFSGSG |
| | | AYMELGSLRSDDTAVYYCARGHQLVP | | SGTDFTLTISSLQPEDFATYYC |
| | | DAFDIWGQGTMVTVSS | | QQSYSTPLTFGGGTKVEIK |
| 07B02 | 4179 | QVQLVQSGGGVVQPGRSLRLSCAASG | 4337 | QSVVTQPPSVSGAPGQRVTISC |
| | | FTFSGYDMHWVRQAPGKGLEWVAVIW | | TGTSSDVGGYNYVSWYQQHPGK |
| | | YDGSNKYYADSVKGRFTISRDNSKNT | | APKLMIYDVSKRPSGVPDRFSG |
| | | LYLQMNSLSAEDTAVYYCARGIAGAA | | SKSGNTASLTISGLQAEDEADY |
| | | FDYWGQGTLVTVSS | | YCLSYDSSLSGSVFGGGTELTV |
| | | | | L |
| 08F03 | 4180 | QVQLQQSGPGLVKPSQTLSLTCAISG | 4338 | QAVLTQPPSVSGAPGQRVTISC |
| | | DSVSSNSAAWNWIRQSPSRGLEWLGR | | TGSSSNIGAGYDVHWYQQLPGT |
| | | TYYRSRWYNDYAVSVKSRITINPDTS | | APKLLIYGNSNRPSGVPDRFSG |
| | | KNQFSLQLNSVTPEDTAVYYCARGKE | | SKSGTSASLAITGLQAEDEADY |
| | | WGHTNTWYGVNYYYMDVWGKGTTVTV | | YCQSYDSSLSGWVFGGGTQLTV |
| | | SS | | L |
| 10E11 | 4181 | QVQLQESGPGLVKPSGTLSLTCAVSG | 4339 | QSVLTQPPSVSGAPGQRVTISC |
| | | GSISSSNWWNWVRQPPGKGLEWIGEI | | TGSSSNIGAGYDVHWYQQLPGT |
| | | YHSGSTSYNPSLKSRVTISVDKSKNQ | | APKLLIYGNSNRPSGVPDRFSG |
| | | FSLKLSSVTAADTAVYYCARGKGYDL | | SKSGTSASLAITGLQAEDEADY |
| | | EYYYYYYMDVWGKGTTVTVSS | | YCQSYDSSLSGSVFGGGTKLTV |
| | | | | L |
| 04F12 | 4182 | QVQLVQSGAEVMRPGASVKVSCKASG | 4340 | AIRMTQSPSSVSASVGDRVTIT |
| | | YTFTNYYIHWVRQAPGQGLEWLGWIN | | CRASQGISSWLAWYQQKPGKAP |
| | | SNSGVTNYAQKFQDRVTMTRDTSIST | | KLLIYAASRLQSGVPSRFSGSG |
| | | AHLELSGLRSDDTAIYYCAKGKWELP | | SGTDFTLAISSLQPEDFATYYC |
| | | DAFDFWGQGTMVTVSS | | QQSYSTPLTFGGGTKVDIK |
| 04D01 | 4183 | QVQLQQSGPGLVKPSQTLSLTCAISG | 4341 | DIVMTQSPSSLSASVGDRVTIT |
| | | DSVSNNGASWNWIRQSPSRGPEWLGR | | CRASQTISSYLNWYQQKPGKAP |
| | | TYYRSGWYNDYAVSVISQITITPDTS | | ELLIYAASSLQSGVPSRFSGSG |
| | | KNQFSLQLNSVTPEDTALYFCARGNI | | SGTDFTLTISSLQPEDFATYYC |
| | | GPAGYHFDYWGQGTLVTVSS | | QQSYGTPLTFGGGTKLEIK |
| 04F04 | 4184 | QVQLQQSGPGLVRPSQTLSLTCAISG | 4342 | AIQMTQSPSSLSASVGDRVTIT |
| | | DSVSSNSAAWNWIRQSPSRGLEWLGR | | CRASQSISSNLNWYQQKPGKAP |
| | | TYYRSDWYNDYGISVTSRATIYPDTS | | KLLIYAASNLQTGVPSRFSGSG |
| | | KNQFSLQLNSVTPEDTAVYYCARGNV | | SGTDETLTISSLQPEDFATYSC |
| | | GPAGYHFDYWGQGTLVTVSS | | QQSYGLPLTFGGGTRLEIK |
| 07D01 | 4185 | QVQLQQWGAGLLKPSETLSLTCAVYG | 4343 | QSALTQPASVSGSPGQSITISC |
| | | GSFSGYYWSWIRQSPGKGLEWIGDIN | | TGTSSDVGGYNYVSWYQQHPGK |
| | | HSGSINYNPSLKSRVTMSVDTSKNHF | | APKLMIYEVNKRPSGVPDRFSG |
| | | SLKLSSVTAADTAVYYCARGPAGTDA | | SKSGNTASLTVSGLQAEDEADY |
| | | FDIWGQGTMVTVSS | | YCSSYAGSNNLVFGGGTKVTVL |
| 10D04 | 4186 | QVQLQESGPGLVRPSGTLSLTCVVSG | 4344 | SYVLTQPPSVSVSPGQTASITC |
| | | GSISSSNWWNWVRQPPGKGLEWIGEI | | SGDKLGNKYASWYQQKPGQSPV |
| | | YHSGSTNYNPSLKSRVTISVDKSKNQ | | LVIYQDSKRPSGIPERFSGSNS |
| | | FSLKLSSVTAADTAVYYCARGPSRDG | | GNTATLTISGTQAMDEADYYCQ |
| | | AGFDYWGQGTLVTVSS | | AWDSSTAVVFGGGTKLTVL |
| 10C01 | 4187 | QVQLQESGPGLVKPSGTLSLTCAVSG | 4345 | SYVLTQPPSVSVAPGKTASITC |
| | | GSISSSNWWNWVRQPPGKGLEWIGEI | | SGDKLGDKYASWYQQKPGQSPV |
| | | YHSGSTNYNPSLKSRVTISVDKSKNQ | | LVIYQDSKRPSGIPERFSGSNS |
| | | FSLKLSSVTAADTAVYYCARGPSVDG | | GNIATLTISGTQAMDEADYYCQ |
| | | AGFDYWGQGTLVTVSS | | AWDSSTAVVFGGGTKLTVL |
| 10E01 | 4188 | QVQLQQSGPGLVKPSGTLSLTCAVSG | 4346 | NFMLTQPPSVSVSPGQTASITC |
| | | GSISSSNWWNWVRQPPGKGLEWIGEI | | SGDKLGNKYVSWYQQKPGQSPV |
| | | YHSGSTNYNPSLKSRVTISVDKSKNQ | | LVIYQDSKRPSGIPERFSGSNS |
| | | FSLKLSSVTAADTAVYYCARGPTGDG | | GNTATLTISGTQAMDEADYYCQ |
| | | AGFDYWGQGTTVTVSS | | AWDSSTAVVFGGGTELTVL |
| 10F07 | 4189 | QVQLVESGGGLVTPGGSLRLSCAASG | 4347 | NFMLTQPPSVSVAPGKTARITC |
| | | FTFNTYSMNWVRQAPGKGLEWVSSIS | | GGNNIGSKSVNWYQQKPGQAPV |
| | | SSGFYIYYADSVKGRFTISRDNAKNL | | LVIYYDSDRPSGIPERFSGFKS |
| | | LYLQMNSLRAEDTAVYYCGGGPTGNY | | GNTATLTISRVEAGDEADYYCQ |
| | | YYFMDVWGKGTTVTVSS | | VWDSSSDHVVFGGGTKVTVL |
| 03B10 | 4190 | QVQLVQSGAEVKRPGASVKVSCKASG | 4348 | DIVMTQSPSSLSASVGDRVTIT |
| | | YTFTGYYMHWVRQAPGQGLEWMGWIN | | CRASQGISRWLDWYQQKPGKAP |
| | | PNSGVTNYSQKFQGRVTMTRDTSIST | | KLLIYAASSLQSGVPSRFSGSG |
| | | AYMELRSLRSDDTAVYYCARGQQLVP | | SGTDFTLTISSLQPEDFATYYC |
| | | DAFDIWGQGTMVTVSS | | QQSYSTPLTFGGGTKVEIK |
| 06E02 | 4191 | EVQLVQSGAEVKKPGASVKVSCKASG | 4349 | DIVMTQSPSSVSASVGDRVTIT |
| | | YTFTGYYIHWVRQAPGQGLEWMGWIN | | CRASQAIRRWLAWYQQKPGKIP |
| | | PISGDTHFAQKFQGRVTMTRDTSIST | | KLLIHSASSLHSGVPSRFSGSG |
| | | AYMELSRLSSDDTAVYHCARGSTWPD | | SGTDFTLTISSLQPEDFATYYC |
| | | VFGIWGQGTMVTVSS | | QQSYSTPLTFGGGTKLEIK |
| 04F03 | 4192 | QVQLQQSGPGLVKPSQTLSLTCAISG | 4350 | DIQLTQSPSPLSASVGDRVTIT |
| | | DSVSNNSAAWNWIRQSPSRGLEWLGR | | CRASQSISSYLNWYQQKPGKAP |
| | | TYYRSKWYNDYAKSRITINPDTSKNQ | | KLLIYAASSLQSGVPSRFSGSG |
| | | ISLQLNSVTPEDTAVYYCARGTIAAA | | SGTDFTLTISSLQPEDFATYYC |
| | | GYYFDYWGQGTLVTVSS | | QQSYSTPLTFGGGTKVEIK |
| 08G03 | 4193 | QVQLVQSGAEVKKPGASVKVSCKASG | 4351 | QAVLTQPRSVSGSPGQSVTISC |
| | | YTFTNYGISWVRQAPGQGLEWMGWIR | | TGTSSDVGGYNYVSWYQQHPGK |
| | | TYNGNTNYAQRFQGRVTMTTDTSTNT | | APKLMIYDVSKRPSGVSNRFSG |
| | | AYMELRSLRSDDTAVYYCARGTLENW | | SKSGNTASLTISGLQAEDEADY |
| | | IDPWGQGTLVTVSS | | YCSSYAGRNNLVFGGGTQLTVL |
| 04H03 | 4194 | QVQLQQSGPGLVKPSQTLSLTCAISG | 4352 | AIQMTQSPSSLSASVGDRVTIT |
| | | DSVSSNSASWNWIRQSPSRGLEWLGR | | CRASQSISRYLNWYQQRPGKAP |
| | | TYYRSNYYNDYAVSVPSRIHINPDTS | | KVLIYAASSLQSGVPSRFSGSG |
| | | KNQFSLQLNSVTPEDTAVYYCARGVV | | SGTDFTLTINSLQPEDFATYYC |
| | | GASGYYMDVWGKGTTVTVSS | | QQSYSTPLTFGGGTKVEIK |
| 06G05 | 4195 | EVQLVQSGAEVKKPGASVKVSCKASG | 4353 | DIVMTQSPSSLSASVGDRVTIT |
| | | YTFTGHYIHWVRQAPGQGPEWMGWSN | | CRASQSIRSYLNWYQQKPGKAP |
| | | PYSGATHYAEKFQGRVTMTLDTSIST | | KLLIYGASSLQRGVPSRFSGSG |
| | | AYMELSRLRSDDTAMYSCARGYNWNP | | SGTEFTLTISSLQPEDFASYYC |
| | | DAFDIWGQGTMVTVSS | | QQSYSSPLTFGGGTKVDIK |
| 12B10 | 4196 | QLQLQESGPGLVKPSETLSLTCTVSG | 4354 | SYVLTQPPSVSVSPGQTARITC |
| | | GSISSSYYYWGWIRQPPGKGLEWIGS | | SGDALPKKYAYWYQQKSGQAPV |
| | | FYYNGFTYYNPSLKSRVTMSVDSSKN | | LVIYEDSKRPSGIPERFSGSSS |
| | | QFSLRLTSVTAADTAVYYCARHEMGA | | GTMATLTISGAQVEDEADYYCY |
| | | NAFDIWGQGTMVTVSS | | STDSSGNHRVFGGGTKVTVL |
| 11G03 | 4197 | QVQLQESGPGLVKPSETLSLTCTVSG | 4355 | SYVLTQPPSVSVSPGQTARITC |
| | | GSISSSYYYWGWIRQPPGKGLEWIGS | | SGDALPKKYAYWYQQKSGQAPV |
| | | FYYNGFTYYNPSLKSRVTMSVDSSKN | | LVIYEDSKRPSGIPERFSGSSS |
| | | QFSLRLTSVTAADTAVYYCARHEVGA | | GTMATLTISGAQVEDEADYYCY |
| | | NAFDIWGQGTMVTVSS | | STDSSGNHRVFGGGTKLTVL |
| 04B12 | 4198 | QVQLQESDPGLVKPSETLSLTCTVSG | 4356 | EIVMTQSPATLSLSPGERATLS |
| | | GSIRSSNYYWGWIRQPPGKGLEWIGN | | CRASQSVSKYLAWYQQKPGQAP |
| | | IYFRGNTYYNPSLKSRVTISVDTSKS | | RLLIYDASNRATGIPARFSGSG |
| | | QFSLTLISVTAADTAVYYCARHGYDS | | SGTDFTLSISSLEPEDFAVYYC |
| | | FDYWGQGTLVTVSS | | QQRSNWPLTFGGGTKLEIK |
| 10H10 | 4199 | QVQLQESGPGLVKPSETLSLTCTVSG | 4357 | QSALTQPASVSGSPGQSITISC |
| | | GSISSSSYYWGWIRQPPGKGLEWIGN | | TGTSSDVGGYNYVSWYQQHPGK |
| | | IFYSGNTYYNPSLKSRVTISVDTSQN | | APKLMIYEVSNRPSGVSNRFSG |
| | | QFSLKLTSVTAADTAVYYCARHYFSS | | SKSGNTASLTISGLQAEDEADY |
| | | SFDYWGQGTLVTVSS | | YCCSYEGSWVFGGGTKLTVL |
| 05G01 | 4200 | EVQLVQSGAEVKKPGASVKVSCKASG | 4358 | DIVLTQSPGTLSLSPGERATLS |
| | | YSFLSYVVSWVRQAPGQGLEWMGWIT | | CRASQSVSSSYLAWYQQKPGQA |
| | | PYNGNTNYAQNLQDRVTMTTDTSTNT | | PRLLIYGASSRATGIPDRFSGS |
| | | AYMELRSLRSDDTAVYYCASKGPTNY | | GSGTDFTLTISRLEPEDFAVYY |
| | | YSYMDVWGKGTTVTVSS | | CQQYGSSPLTFGGGTKVEIK |
| 10H01 | 4201 | QVQLVQSGGGVVQPGRSLRLSCAASG | 4359 | QSVLTQPPSVSGAPGQRVTISC |
| | | FTFSGHDMHWVRQAPGKGLEWVGVIW | | TGSSSKIGAGYDVHWYQQLPGT |
| | | YDGSNKYYGDSVKGRFTISRDNSKNT | | APKLLIYGNSNRPSGVPDRFSG |
| | | LYLQMNSLRAEDTAVYYCARKTQFGF | | SKSGTSASLAITGLQAEDEADY |
| | | DYWGQGTTVTVSS | | YCQSYDSSLSGSVFGGGTKVTV |
| | | | | L |
| 11B03 | 4202 | EVQLVESGGGLVKPGGSLRLSCAASG | 4360 | QAVVTQDPAVSVALGQTVRITC |
| | | FTFSNYAMHWVRQAPGKGLEWVAVIW | | QGDSLRNYYASWYQQRPGQAPV |
| | | YDGSNKYYADSVKGRFTISRDNSKNT | | LVIYGNNNRPSGIPDRFSGSRS |
| | | GYLQMNSLRAEDTAVYYCATLAADAP | | GNSASLTITGAQAEDEADYYCN |
| | | KWFDPWGQGTLVTVSS | | SRDSSGNHVVFGGGTKVTVL |
| 08H01 | 4203 | QVQLQESGPGLVKPSETLSLTCTVSG | 4361 | QSVLTQPPSVSGAPGQRVTISC |
| | | GSISSSSYYGGWIRQPPGKGLEWIGS | | TGSSSNIGAGYDVHWYQQLPGT |
| | | IYYSGSTYYNPSLKSRVTISGDTSKN | | APKLLIYGNSNRPSGVPDRFSG |
| | | QFSLKLSSVTAADTAVYYCARLYNWN | | SKSGTSASLAITGLQAEDEADY |
| | | YGGGFFDYWGRGTLVTVSS | | YCQSYDSSLSGWVFGGGTKVTV |
| | | | | L |
| 05D06 | 4204 | QLQLQESGPGLVKPSQTLSLTCTVSG | 4362 | AIQMTQSPSSVSASVGDRVTIT |
| | | GSVSSGGYYWSWIRQYPGKGLEWIGY | | CRASQGLRNWLAWYQQKPGKAP |
| | | IHHRGTTYFNPSLKSRVTMSLDTSKN | | NLLISAASGLQGGVPSRFSGSG |
| | | QFSLKLSSVTAADTAVYYCAGMVRGI | | SGTDFTLTISSLQPEDFATYYC |
| | | PLYYYFMDVWGKGTTVTVSS | | QQANIFPLTFGGGTKLEIK |
| 05D11 | 4205 | QVQLQESGPGLVKPSQTLSLTCTVSD | 4363 | DIVMTQSPSSVSASVGDRVTIS |
| | | GSISSGVYYWSWIRQHPGKGLEWIGY | | CRASQGISSWLVWYQQKPGKAP |
| | | ISNSETTYYNPSLKSRVTISVDTSKN | | NLLIDAASSLQSGVPSRFSGSR |
| | | QFSLNLTSVTAADTAVYYCARMVRGL | | SGTDFTLTISNLQPEDFATYYC |
| | | DYYYYYMDVWGKGTLVTVSS | | QQANSFPLTFGGGTKLEIK |
| 05C06 | 4206 | QVQLQESGPGLVKPSGTLSLTCAVSG | 4364 | DIQLTQSPSSVSASVGDRVTIT |
| | | GSISSGDYYWSWIRQHPGKGLDWIGY | | CRASQGISSWLAWYQQKPGKAP |
| | | IYYRGTTYYNPSLKSRVTISADTSKN | | KLLIYAASSLQSGVPSRFSGSG |
| | | QFSLKLSSVTAADTAVYYCARMVRGL | | SGTDFTLTISGLQPEDFATYYC |
| | | KYYYYYMDVWGKGTTVTVSS | | QQANSFPLTFGGGTKVEIK |
| 05H02 | 4207 | QVQLVESGPGLVKPSQTLSLTCTVSG | 4365 | DIVMTQSPSSVSASVGDRVTIT |
| | | GSISSGDYYWSWIRQHPGKGLEWIGY | | CRASQGISSWLAWYQQKPGKAP |
| | | IYYRGTTYYNPSLKSRVAISVDTSKN | | KLLIYAASSLQSGVPSRFSGSG |
| | | QFSLKLSSVTAADTAVYYCARMVRGL | | SGTDFTLTISSLQPEDFATYYC |
| | | NYYYYHMDVWGKGTTVTVSS | | QQANSFPLTFGGGTKVEIK |
| 05E03 | 4208 | QLQLQESGPGLVKPSQTLSLTCTVSG | 4366 | DIVMTQSPSSVSASVGDRVTIT |
| | | GSLSSGDYYWSWIRQHPGKGLEWIGY | | CRASQGISSWLAWYQQKPGKAP |
| | | ISYIGTTYYNPSLKSRVTISEDTSKN | | KLLIYAASSLQSGVPSRFSGSG |
| | | QFSLKLSSVSAADTAVYYCARMVRGL | | SGTDETLTISSLQPEDFATYYC |
| | | NYYYYMDVWGKGTTVTVSS | | QQANSFPITFGQGTRLEIK |
| 05D01 | 4209 | QVQLVESGPGLVKPSQTLSLTCAVSG | 4367 | DIQMTQSPSSVSASVGDRVTIT |
| | | GSISSGDYYWSWIRQLPGKGLEWIGY | | CRASQGISSWLAWYQQKPGKAP |
| | | IYYRGTTFYNPSLKSRVTISGDTSKN | | KLLIYAASSLQSGVPSRFSGSG |
| | | QFSLKLNSVTAADTAVYYCARMVRGL | | SGTDFTLTISSLQPEDFATYYC |
| | | NYYYYYMDAWGKGTTVTVSS | | QQANSFPPTFGQGTKVDIK |
| 05B04 | 4210 | QVQLQESGPGLVKPSQTLTLTCTVSG | 4368 | DIVMTQSPSSVSASVGDRVTIT |
| | | GSITSGDYYWSWIRQHPGKGLEWIGY | | CRASQGISSWLAWYQQKPGKAP |
| | | IYYRGTTYYNPSLKSRVTISLDTSKN | | KLLIYAASSLQSGIPSRFSGSG |
| | | QFSLKLSSVTAADTAVYYCARMVRGL | | SGTDFTLTISSLQPEDFATYYC |
| | | NYYYYYMDVWGKGTTVTVSS | | QQANSFPPTFGPGTKVEIK |
| 05E01 | 4211 | QVQLQESGPGLVKPSQTLSLICTVSG | 4369 | DIQLTQSPSTLSASVGDRVTIT |
| | | DSISSGDYYWSWIRQHPGKGLEWIGY | | CRASQGISSWLAWYQQKPGKAP |
| | | IYYRGTTYYNPSLKSRVTISADTSKN | | KLLIYAASSLQSGVPSRFSGSG |
| | | QFSLKLSSVTAADTAVYYCARMVRGL | | SGTDFTLTISSLQPEDFATYYC |
| | | NYYYYYMDVWGKGTTVTVSS | | QQANSFPPTFGQGTKVDIK |
| 05G03 | 4212 | QLQLQESGPGLVKPSQTLSLTCTVSG | 4370 | DIQMTQSPSSVSASVGDRVTIT |
| | | GSISSNGYYWTWIRQHPGRGLEWIGY | | CRASQGLSNWLAWYQQKPGEAP |
| | | MSYIGTTYSNPSLKSRVSISGDTSDN | | KLLISAASSLQSGVPSRFSGSG |
| | | QFSLSLTSVTAADTAVYYCARMVRGL | | SGTEFTLTISSLQPEDFATYYC |
| | | TYYYYMDVWGKGTTVTVSS | | QQANGFPITFGGGTKLEIK |
| 05C01 | 4213 | QVQLVESGPGLVKPSQTLSLTCTVSG | 4371 | DIVMTQSPSSVSASVGDRVTIT |
| | | GSISSGVYYWSWIRQHPGKGLEWIGY | | CRASQGISRWLAWYQQKPGKAP |
| | | ISYSGTTYYNPSLKSRVTISEDTSKN | | KLLIYAASSLQSGVPSRFSGSG |
| | | QFSLKLSSVSAADTAVYYCARMVRGV | | SGTEFTLTISSLQPEDFATYYC |
| | | DYYYYYMDVWGKGTTVTVSS | | QQANSFPLTFGGGTKLEIK |
| 05E07 | 4214 | QVQLQESGPGLVKPSQTLSLTCTVSD | 4372 | DIQLTQSPSSVSASVGDRVTIT |
| | | GSISSGVYYWSWIRQHPGKGLEWIGY | | CRASQGLSNWLAWYQQKPGKAP |
| | | ISNSGTTYYNPSLKSRVTISVDTSKN | | NLLISAASGLQGGVPSRFSGSG |
| | | QFSLKLSSVTAADTAVYYCARMVRGV | | SGTDFTLTISSLQPEDFASYYC |
| | | NYYYYMDVWGKGTTVTVSS | | QQANIFPLTFGGGTKVEIK |
| 05B02 | 4215 | QLQLQESGPGLVKPSQTLSLTCTVSD | 4373 | DIQMTQSPSSVSASVGDRVTIT |
| | | GSISSGVYYWSWIRQHPGKGLEWIGY | | CRASQGISSWLAWYQQKPGKAP |
| | | ISNSGTTYYNPSLKSRVTISVDTSKN | | KLLIYAASSLQSGVPSRFSGSG |
| | | QFSLKLSSVTAADTAVYYCARMVRGV | | SGTDFTLTISSLQPEDFATYYC |
| | | NYYYYYMDVWGKGTTVTVSS | | QQANSFPLTFGGGTKVEIK |
| 06C02 | 4216 | QVQLQESDAGLVRPSETLSLTCTVSG | 4374 | EIVMTQSPATLSLSPGERATLS |
| | | DSIRRTNYYWAWIRQPPGKGLEWIGN | | CRASQSVSSYLAWYQQKPGQAP |
| | | IYYSGDTYYNPSLKSRVTMSVDTSKN | | RLLIYDASNRATGIPARFSGSG |
| | | QFSLKLSSVTAADTAVYYCARNGYDS | | SGTDFTLTISSLEPEDFAVYYC |
| | | FDYWGQGTLVTVSS | | QQRSNWPLTFGGGTKLEIK |
| 05C04 | 4217 | QVQLVQSGGGLVKPGGSLRLSCEASG | 4375 | DVVMTQSPLSLPVTLGQPASIS |
| | | FTFSTYSMNWVRQAPGKGLEWVSSIG | | CRSSQSLVYSDGNTYLNWFQQR |
| | | GSSSYTYYADSVKGRFTISRDNAKNS | | PGQSPRRLIYKVSNRDSGVPDR |
| | | LYLQMNSLRAEDTAVYYCARQGYYDS | | FSGSGSGTDFTLKISRVEAEDV |
| | | SGYYFPDAFDIWGQGTMVTVSS | | GVYYCMQGTHWPFTFGPGTKLE |
| | | | | IK |
| 03G04 | 4218 | QLQLQEWGAGLLKPSETLSLTCAVYG | 4376 | DIVMTQSPSSLSASVGDRVTIT |
| | | GSLSGYYWSWIRQPPGKGLEWIGEIN | | CRASQGIRNDLGWYQQKPGKAP |
| | | HYGSTNYNPSLKSRVTISADTSKNQF | | KLLIYAASSLQSGVPSRFSGSG |
| | | SLRLNSVTAADTAVYYCARQNWNYEG | | SGTDFTLTISSLQPEDFATYYC |
| | | FDYWGQGTLVTVSS | | LQDYNYPYTFGQGTKLEIK |
| 09B05 | 4219 | EVQLVQSGAEVKKPGESLKISCKGSG | 4377 | QPVLTQPASVSGSPGQSITISC |
| | | YSFTSYWIGWVRQMPGKGLEWMGIIY | | TGTSSDVGGYNYVSWYQQHPGK |
| | | PGDSATRYSPSFQGQVTISVDKSINT | | APKLMIYDVSKRSSGVSNRFSG |
| | | AYLQWGSLKASDTAMYYCVRQPLNYD | | SKSGNTASLTISGLQAEDEADY |
| | | WFDPWGQGTLVTVSS | | YCCSYAGSSTWVFGGGTKVTVL |
| 02D06 | 4220 | EVQLVQSGAEVKKPGASLKVSCKASG | 4378 | DIVMTQSPSSLSASVGDRVTIT |
| | | YNFKSFGFSWMRQAPGQGLEWMGWTS | | CRASQSISSYLNWYQQKPGKAP |
| | | GYNGITKYSQMFQDRLTLTSDTSTST | | KLLIYAASSLQSGVPSRFSGSG |
| | | VYMEMRSLRSDDTAVYYCASRDNWNY | | SGTDFTLTISSLQPEDFATYYC |
| | | DAFDLWGQGTMVTVSS | | QQAYSFPYTFGQGTKLEIK |
| 08A03 | 4221 | QVQLQESGPGQVKPSETLSLTCTVSD | 4379 | SYELTQPPSVSVSPGQTARITC |
| | | GSISSSTFYWGWIRQPPGKGLEWIGS | | SGDALPKKYAYWYQQKSGQAPV |
| | | IYYSGNTYYNPSLKSRVTLSVDKSKK | | LVIYEDSKRPSGIPERFSGSSS |
| | | QFSLKLSSVTAADTAVYYCARRDWNH | | GTMATLTISGAQVEDEADYYCY |
| | | IDYWGQGTTVTVSS | | STDSSGNHYVFGTGTKVTVL |
| 06G08 | 4222 | QITLKESGPALVKPTQTLTLTCTESG | 4380 | EIVMTQSPATLSLSPGERVTLS |
| | | FALTTSGEGVGWIRQPPGKALEWLAL | | CRASQSIGSYLAWYQQKPGQAP |
| | | IYWNDDKRYSPSLKNRVAITKDTSKN | | RLLIYDASNRATGIPARFSGSG |
| | | QVVLTMANMDPVDTATYYCVHRGFKS | | SGTDFTLTISSLEPEDFAVYYC |
| | | RWYVDYWGQGTLVTVSS | | QQRSNWPFTFGPGTKLEIK |
| 05B01 | 4223 | QVTLRFSGPTLVKPTQTLTLTCTESG | 4381 | EIVLTQSPATLSLSPGERATLS |
| | | FSLTTSGEGVGWIRQPPGKAPEWLAL | | CRASQSVSSYLAWYQQKPGQAP |
| | | IYWNDDKRYNPSLENRFTITKDTSKN | | RLLIYDASNRATGIPARFSGSG |
| | | QVILTMTNLDPVDTATYYCAHRGERS | | SGTDFTLTISSLEPEDFAVYYC |
| | | RWYVDFWGQGTLVTVSS | | QQRSNWPFTFGPGTKVEIK |
| 10A01 | 4224 | QVQLQESGPGLVKPSETLSLTCTVSG | 4382 | QAVLTQPPSASGTPGQRVTISC |
| | | GSISSTGFFWGWIRQPPGKGLDWIGS | | SGSSSNIGSNTVNWYQQLPGTA |
| | | IYYSGNTYYNPSLKSRVTISVDTSKN | | PKLLIYSNNQRPSGVPDRFSGS |
| | | QFSLKLSSVTAADTAVYYCARRSPRW | | KSGTSASLAISGLQSEDEADYY |
| | | SSSGSDAFDIWGQGTMVTVSS | | CAAWDDSLNGWVFGGGTKVTVL |
| 02G05 | 4225 | QVQLVQSGAEVKKPGVSVKVSCKASG | 4383 | AIRMTQSPSSLSASVGDRVTIT |
| | | YTFTSYGISWVRQAPGQGLEWMGWIS | | CRASQISSRHLNWYQQKPGKAP |
| | | AYDGDTNYAQKFQGRVNMTTDTFTST | | KLLIYAASSLQGGVPSRFSGSG |
| | | AYMELRSLRSDDTAVYYCARRWNYEK | | SGTDFTLTISSLQPEDFATYYC |
| | | GAFDIWGQGTMVTVSS | | QQSYSTPFTFGPGTKLEIK |
| 02E01 | 4226 | EVQLVQSGAEVKKPGASVKVSCKASG | 4384 | AIQMTQSPSSLSASVGDRVTIT |
| | | YNFTSYGISWVRQAPGQGLEWMGWTS | | CRASQSISSYLNWYQQKPGKAP |
| | | GYNGITKYAQKFQDRLTMTSDTSTST | | KLLIYAASSLQSGVPSRFSGSG |
| | | VYMELRSLRSDDTAVYYCASRYNWNY | | SGTDFTLTISSLQPEDFATYYC |
| | | AAFDLWGQGTMVTVSS | | QQSYSTPLTFGGGTKVEIK |
| 05C03 | 4227 | EVQLVQSGAEVKKPGASVKVSCKASG | 4385 | AIQMTQSPSSLSASVGDRVTIT |
| | | YTFTNYGVSWVRQAPGQGLEWMGWIG | | CRASQSISSYLNWYQQKPGKAP |
| | | ANNAYTKYAQKLQGRVTMTTDTSTST | | KLLIYAASSLQSGVPSRFSGSG |
| | | AYMDLRSLRSDDTAIYFCASRYNWNY | | SGTDFTLTISSLQPEDFATYYC |
| | | DAFDIWGQGTMVTVSS | | QQSYSTPLTFGGGTKLEIK |
| 08E05 | 4228 | QVQLVQSGAEVKKPGASVKVSCKASG | 4386 | QAVVTQDPAVSVALGQTVRITC |
| | | YTLTNYGVSWVRQAPGQGLEWMGWIS | | QGDSLRSYYASWYQQKPGQAPV |
| | | AYNGDTNYAQKFQGRVTMTTDTSTST | | LVIYGKNNRPSGIPDRFSGSSS |
| | | AYMELRSLISDDTAVYYCARRYNWNY | | GNTASLTITGAQAEDEADYYCN |
| | | GIFDNWGQGTLVTVSS | | SRDSSGNHVVFGGGTKVTVL |
| 08H07 | 4229 | QVQLVQSGAEVKKPGASVKVSCKASG | 4387 | QTVVTQDPAVSVALGQTVRITC |
| | | YTFANYGISWVRQAPGQGLEWMGWIS | | QGDSLRSYYASWYQQKPGQAPV |
| | | AYNGNTNYAQKFQGRVTLTTDTSTST | | LVIYGKNNRPSGIPDRFSGSSS |
| | | AYMELRSLRSDDTAVYYCARRYNWNY | | GNTASLTITGAQAEDEADYYCN |
| | | GILDYWGQGTLVTVSS | | SRDSSGNHVVFGGGTKLTVL |
| 08B04 | 4230 | EVQLVQSGAEVKKPGASVKVSCKASG | 4388 | QTVVTQDPAVSVALGQTVRITC |
| | | YTFTSYGITWVRQAPGQGLEWMGWIS | | QGDSLRSYYASWYQQKPGQAPV |
| | | VYNGDTNYAQNLQGRVTMTTDTSTNT | | LVIYGKNNRPSGIPDRFSGSSS |
| | | AYMELRSLRSDDTAMYYCARRYNWNY | | GNTASLTITGAQAEDEADYYCN |
| | | GLFDYWGQGTLVTVSS | | SRDSSGNHLVFGGGTKVTVL |
| 11F01 | 4231 | QVQLVQSGAEVKNPGASVKVSCKASG | 4389 | QTVVTQDPAVSVALGQTVRITC |
| | | YTLTNYGISWVRQAPGQGLEWMGWIS | | QGDSLRSYYASWYQQKPGQAPV |
| | | AYNGDTNYAQKIQGRVTMTTDTSTST | | LVIYGKNNRPSGIPDRFSGSSS |
| | | AYMELRSLRSDDTAVYYCARRYNWNY | | GNTASLTITGAQAEDEADYYCN |
| | | GLLDYWGQGTLVTVSS | | SRDSSGNHVVFGGGTKLTVL |
| 02E06 | 4232 | QVQLVQSGPEVKKPGASLKVSCKASG | 4390 | DIQLTQSPSSLSASVGDRVTIT |
| | | YNFTSFGINWVRQAPGQGLEWMGWTS | | CRASQSISSYLNWYQQKPGKAP |
| | | GYNGITKYVEKFQDRLTMTSDTSTGT | | KLLIYAASSLQSGVPSRFSGSG |
| | | VYMELRSLRSDDTAVYYCASRYNWNY | | SGTDFTLTISSLQPEDFATYYC |
| | | VAFDLWGQGTMVTVSS | | QQSYSTPLTFGGGTKVEIK |
| 10C02 | 4233 | QVQLQESGPGLVKPSGTLSLTCAVSG | 4391 | SYELTQPPTVSVSPGQTASITC |
| | | GSISNSNWWTWVRQPPGKGLEWIGEI | | SGDKLGDKYASWYQQKPGQSPV |
| | | YHSVNTNYNPSLKSRVTISVDKSKNQ | | LVIYQDNKRPSGIPERFSGSNS |
| | | FSLNLSSVTAADTAVYYCARSPYGGN | | GNTATLTISGTQAMDEADYYCQ |
| | | PYFDYWGQGTLVTVSS | | AWDSSTVVFGGGTKVTVL |
| 03G02 | 4234 | EVQLVQSGAEVKKPGASVKVSCKASG | 4392 | DIQMTQSPSFLSASVGDRVTIT |
| | | YTFTGYYIHWVRQAPGQGLDWMGWIN | | CRASQNINSYLNWYQQKPGKAP |
| | | PYSGATNYAQKFQGRVTMTRDTSLST | | NLLIYAASSLQSGVPSRFSGSG |
| | | AYMELSRLRSDDTAVYYCAKSRLPDA | | SRTDFTLTISSLQPEDFATYYC |
| | | FDIWGPGTMVTVSS | | QQSYSTPITFGQGTRLEIK |
| 06F09 | 4235 | EVQLVQSGAEVKKPGASVKVSCKASG | 4393 | DIQMAQSPSSLSASVGDRVTIT |
| | | YTFTNLGISWVRQAPGQGLEWMGWIS | | CRASQSISSYLNWYQQKPGKAP |
| | | PYNGFTNYAQKLQGRVTMTTDTSTST | | KLLIYAASSLQSGVPSRFSGSG |
| | | AYMELRSLRSDDTAVYYCARTEPLDY | | SGTDFTLTISSLQPEDFATYYC |
| | | WGQGTLVTVSS | | LQDSNYPLTFGGGTKVEIK |
| 06G02 | 4236 | QVQLVQSGAEVKKPGASVKVSCKASG | 4394 | DIVMTQSPSSLSASVGDRVTIT |
| | | YTFTNYGISWVRQAPGQGLEWMGWIS | | CRASQSISSYFNWYQQKPGKAP |
| | | AYNGNTNYAQKFQGRVTMTTDTSTNT | | KLLIYAASSLQSGVPSRFSGSG |
| | | AYMELRSLRSDDTAVYYCARTWNSDY | | SGTDFTLTISSLQPEDFATYYC |
| | | WGQGTTVTVSS | | QQTDSSPWTFGQGTKVDIK |
| 06D01 | 4237 | QVQLVQSGAEVKKPGASVKVSCKASG | 4395 | DIQLTQSPSSLSASVGDRVTIT |
| | | YTFTNYGISWVRQAPGQGLEWMGWIS | | CRASQSISSYLNWYQQKPGKAP |
| | | VYNGYTKYSQKLQGRVTMTTDTSTNT | | KLLIHAASSSQSGVPSRFSGSG |
| | | AFMELRGLRSDDTAVYYCARTYAFDI | | SGTDFTLTISSLQPEDFATYYC |
| | | WGQGTMVTVSS | | QQSYSTPFTFGPGTKVEIK |
| 07D10 | 4238 | EVQLVQSGAEVKKPGASVKVSCKASE | 4396 | QSALTQPRSVSGSPGQSVTISC |
| | | YTFTSYNMHWVRQAPGQGLEWMGIIH | | TGTSSDVGGYNYVSWYQQHPGK |
| | | PSGGSTNYAQKFQGRVTMTRDTSTST | | APKLMIYDVSKRPSGVPDRFSG |
| | | VYLELSSLRSEDTAVYYCEAVGDYFD | | SKSGNTASLTISGLQAEDEADY |
| | | YWGQGTLVTVSS | | YCCSYAGSYTWVFGGGTKVTVL |
| 02E03 | 4239 | EVQLVQSGAEVKKPGASVKVSCKASG | 4397 | DIVMTQSPSSLSASVGDRVTIT |
| | | YTFTTYGISWVRQAPGQGLEWMGWIS | | CRASQSISSYLNWYQQKPGKAP |
| | | AYYGNTNYAQKLQGRVTMTTDTSTST | | KLLIYAASSLQSGVPSRFSGSG |
| | | AYMELRSLRSDDTAVYYCARVRYSSS | | SGTDFTLTISSLQPEDFATYYC |
| | | WGWYFDLWGRGTLVTVSS | | QQSYSTPLTFGGGTKLEIK |
| 05C05 | 4240 | QVQLQQSGPGLVKPSQTLSLTCVISG | 4398 | DVVMTQSPLSLPVTLGQPASIS |
| | | DSVSSNSAAWNWIRQSPSRGLEWLGR | | CRSSQSLVYSDGNTYLNWFQQR |
| | | TYYMSKWFTEYAVSVKRRITINADTS | | PGQSPRRLIYKVSNRDSGVPDR |
| | | KNQFSLQLDSVTPEDTAVYYCARWDL | | FSGSGSGTDFTLKISRVEAEDV |
| | | GSLDYWGQGTLVTVSS | | GVYYCMQGTHWPYTFGQGTKLE |
| | | | | IK |
| 05D05 | 4241 | QVQLVESGPGLVKPSGTLSLTCAVSG | 4399 | DIVMTQSPSSLSASVGDRVTIT |
| | | DSISSNNWWSWVRQPPGKGLEWIGEI | | CRASQSTSSYLNWYQQKPGKAP |
| | | YHGGSTNYNPSLKSRVTMSVDKSKNQ | | KLLIYAASSLQSGVPSRFSGSG |
| | | FSLKLTSVTAADTAVYYCARWNIELF | | SGTDFTLTISSLQPEDFATYYC |
| | | RGYFDYWGQGTLVTVSS | | QQSYSTPFTFGPGTKLEIK |
| 05A06 | 4242 | QVQLQESGPGLVKPSGTLSLTCAVSG | 4400 | AIRMTQSPSSLSASVGDRVTIT |
| | | GSISGSDWWSWVRQPPGKGLEWIGEI | | CRASQSISSYLNWYQQKPGKAP |
| | | FHSGSTNYNPSLKSRVNFSVDKSKNQ | | KLLIFAASSLQSGVPSRFSGSG |
| | | FSLRLNSVTAADTAVYYCARWNIENF | | SETDFTLTISSLQPEDFASYYC |
| | | AGYFDLWGRGTMVTVSS | | QQSFSTPFTFGPGTKLEIK |
| 05H01 | 4243 | QVQLVESGPGLVQPSGTLSLTCAVSG | 4401 | AIRMTQSPSSLSASVGDRVTIT |
| | | DSISSSNWWSWVRQPPGKGLEWIGEI | | CRASQSISSYLNWYQQKPGKAP |
| | | YHGGSTSYNPSLKSRITMSVDRSKNQ | | KLLIYAASSLQSGVPSRFSGSG |
| | | FSLKLTSVTAADTAVYYCARWNITLF | | SGTDFTLTISSLQPEDFATYYC |
| | | RGYFDYWGQGTLVTVSS | | QQSYSTPFTFGPGTKLEIK |
| 02D01 | 4244 | QVQLQESGPGLVKPSGTLSLTCAVSG | 4402 | DIVMTQSPSSLSASVGDRVTIT |
| | | GSISSSNWWSWVRQSPGKGLEWIGEI | | CRASQSISSYLNWYQQKPGKAP |
| | | YHSGSTNYNPSLRSRVTISLDKSKNQ | | KLLIYAASSLQSGVPSRFSGSG |
| | | FSLKLSSVTAADTAAYYCARWNIVGA | | SGTDFTLTISSLQPEDFATYYC |
| | | LGYFDLWGRGTLVTVSS | | QQSYSTPFTFGPGTKLEIK |
| 10G05 | 4245 | QLQLQESGPGLVKPSRTLSLTCAVSG | 4403 | QSALTQPASVSGSPGQSITISC |
| | | GSISSTNWWNWVRQSPGKGLEWIGEI | | TGTSSDVGGYNYVSWYQQHPGK |
| | | YHSGTTNHNPSLKSRVTMSVDKSKNQ | | APKLMIYEVSNRPSGVSNRFSG |
| | | FSLQLSSVTAADTAVYYCAKYYYGSG | | SKSGNTASLTISGLQAEDEADY |
| | | SYAFDIWGQGTMVTVSS | | YCSSYTSSSTWVFGGGTKVTVL |

TABLE 18
VH-CDR1, VH-CDR2, VH-CDR3 Sequences for EPOR/CD131 Binders IV
(Phage Library) of Table 17

| Clone | SEQ | CDRH1 | SEQ | | SEQ | |
|---|---|---|---|---|---|---|
| ID | ID NO | Sequence | ID NO | CDRH2 Sequence | ID NO | CDRH3 Sequence |
| 06C01 | 4668 | GNYIH | 4826 | WINPYSDATHYAQKFQG | 4984 | AARPDWFDP |
| 06H04 | 4669 | DNYIH | 4827 | WINPYSGATHYAQNFQD | 4985 | AARPDWFDP |
| 10H09 | 4670 | NYYMH | 4828 | WIIPNSGGTKFAQKFQG | 4986 | AAYSSSWYDY |
| 03G03 | 4671 | NHFMH | 4829 | IINPSGGSTNYAQKFQG | 4987 | ADGYEAFDI |
| 11F05 | 4672 | GYYIH | 4830 | WIIPNSGGTKYAQKFQG | 4988 | AGFSSSWYDY |
| 07H07 | 4673 | GYYMH | 4831 | WINPHSGGTNYAQKFQG | 4989 | AGICSGTTCYGYGSDY |
| 08D01 | 4674 | GYYMH | 4832 | WINPHSGGTNYAQKFQG | 4990 | AGICSGTTCYGYGSDY |
| 11B05 | 4675 | GYYIH | 4833 | WIIPNSGGTKYAQNFQG | 4991 | AGYSSAWYDY |
| 11A03 | 4676 | DYYMH | 4834 | WIIPNSGGTKYAQNFQG | 4992 | AGYSSSWYDN |
| 11H08 | 4677 | NYYIH | 4835 | WIIPNSGGTKYAQKFQG | 4993 | AGYSSSWYEY |
| 10F01 | 4678 | GYDMH | 4836 | VIWYDGSNKYYADSVKG | 4994 | AIAAAGFDY |
| 08C09 | 4679 | GNYIH | 4837 | WIHPYSGGTKYAQKFQG | 4995 | APAGTPYFDY |
| 10F08 | 4680 | GYYMH | 4838 | WIIPNSGGTKYAQKFQG | 4996 | ASYSSSWYDY |
| 11F06 | 4681 | GYYIH | 4839 | WIIPNSGGTKYAQKFQG | 4997 | AYINSAYFDY |
| 05H05 | 4682 | DYGMS | 4840 | GINWNGDSKGYVDSVKG | 4998 | DDFYYYMDA |
| 06A09 | 4683 | SYSMN | 4841 | VISGSGGGTFYADSVKG | 4999 | DDWNYEAFDI |
| 01E06 | 4684 | NYGIS | 4842 | WISAYNGNTNYAQKLQG | 5000 | DDYYYDGSGFDF |
| 02F06 | 4685 | DYGMS | 4843 | GINWNGDSTGYVDSVKG | 5001 | DDYYYYMDV |
| 04B04 | 4686 | SYYMH | 4844 | VISPTGGGTHYAQKFQG | 5002 | DEAGDDAFDI |
| 04D11 | 4687 | SYYMH | 4845 | VISPTGGGTHYAQKFQG | 5003 | DEAGDDAFDI |
| 04D07 | 4688 | SYYMH | 4846 | VISPTGGGTHYAQKFQG | 5004 | DEAGDGAFDI |
| 06C09 | 4689 | STNYYWG | 4847 | SIYYSGITYYNPSLKS | 5005 | DGWDAFDI |
| 08F05 | 4690 | SNSATWN | 4848 | RTYYRSKWYNDYAVSVKS | 5006 | DGWNDDEDY |
| 10F04 | 4691 | SHSGAWN | 4849 | RTYYRSKWYNDYAVSVKS | 5007 | DGWTDAFDI |
| 09F01 | 4692 | NAWMS | 4850 | RIKSKTDGGTTDYAAPVKG | 5008 | DHPYY |
| 05H09 | 4693 | SNIAAWN | 4851 | RTYYRSKWYNEYAVSVKG | 5009 | DLHSSTLDY |
| 06C12 | 4694 | SNSTAWN | 4852 | RTYYWSKWYNEYAVSVKG | 5010 | DLHSSTLDY |
| 07C09 | 4695 | SYAMT | 4853 | AISGSGGQTFSADSVKG | 5011 | DLKWEILPDAFDI |
| 07E03 | 4696 | NYAMT | 4854 | AITGSGGNTFSADSVKG | 5012 | DLKWELLPDAFDI |
| 02H08 | 4697 | GYDVN | 4855 | GISSSGESTYYADSVKG | 5013 | DMGFNWNYGAFNM |
| 06D02 | 4698 | GYYMH | 4856 | WINPNSGGTNYAQKFQG | 5014 | DNWNSFDF |
| 07E04 | 4699 | DYYMS | 4857 | YISGPGSTIYYADSVKG | 5015 | DNWNYGFDY |
| 02C03 | 4700 | SNNWWS | 4858 | EIYHNGTTSYNPSLES | 5016 | DQEGLTGCHDY |
| 08F02 | 4701 | SYAMN | 4859 | WINTNTGNPTYAQGFTG | 5017 | DRNYYGSGSYYGDY |
| 06F01 | 4702 | RYAVN | 4860 | GIIPIFGTVNYAQKFQG | 5018 | DRSGTTNWFFDL |
| 10A04 | 4703 | NYAMN | 4861 | AILGSGGNTYYADSVKG | 5019 | DRSNWNFGDWFDP |
| 07C08 | 4704 | NYAMN | 4862 | WINTNTGNPTYAQGFTG | 5020 | DRYYYGSGTYFFDF |
| 04B08 | 4705 | NYGIS | 4863 | WISAYNGNTNYAQKLQG | 5021 | DSRGWFDP |
| 07D05 | 4706 | SYSMN | 4864 | YISSSSSTIYYADSVKG | 5022 | DVGGNYYYNMDV |
| 03C12 | 4707 | GYYMN | 4865 | LISPNSGDTYYSHKFQG | 5023 | DYGDYDGMDV |
| 07G09 | 4708 | GYYMH | 4866 | WINPNSGDTNYEQKFQG | 5024 | EDIAAVGENYYYYYMDV |
| 07H01 | 4709 | GYYMH | 4867 | WINPNSGDTNYEQKFQG | 5025 | EDIAAVGENYYYYYMDV |
| 07B01 | 4710 | GYYLH | 4868 | WIDPNSGGINSAQIFQG | 5026 | EDTWNFVPYFDY |
| 07A01 | 4711 | GSYIH | 4869 | WINPNSGATNYAQRFQG | 5027 | EGIAPVDTGYYYYMDV |
| 09F09 | 4712 | STNWWS | 4870 | AIYQTGNTYYNPSLKS | 5028 | EGLYFGDLLSYAFDI |
| 07C01 | 4713 | GNYIH | 4871 | WINPNSGHTNYAQNFQG | 5029 | EGVAAAGTGFFYYMDV |
| 06G01 | 4714 | WYWMH | 4872 | RIKSDGSNTRYADSVKG | 5030 | EGWGINGLGGAFDI |
| 06A01 | 4715 | WHWMH | 4873 | RIKSDGSNTKYADSVKG | 5031 | EGWGISGLGGAFDI |
| 06B06 | 4716 | WFWLH | 4874 | RIKSDGSSTKYADSVKG | 5032 | EGWGITGQGGALDI |
| 06A02 | 4717 | YYWLH | 4875 | RINSDGSNTKYADSVKG | 5033 | EGWGMTGLGGAFDI |
| 06F12 | 4718 | WYWMH | 4876 | RIKSDGGSPKYADSVKG | 5034 | EGWGMTGLGGAFDI |
| 05D12 | 4719 | NYTMN | 4877 | SISSSGTYIYYADSVKG | 5035 | EGYYDFLTGYISDAFDI |
| 05D07 | 4720 | RYRMN | 4878 | SISSSTSYIFYADSVKG | 5036 | EGYYNNSGYYFPDAFDI |
| 02F04 | 4721 | DYYVY | 4879 | WINPDSGGTNYEQKFQG | 5037 | EKGLRYSGYDRGYFDY |
| 02A07 | 4722 | DYYIH | 4880 | WINPNSGGTHYAQKFQG | 5038 | EMGLRYSGYDRGYFDY |
| 07C02 | 4723 | SNNWWS | 4881 | EIYHSGSTSYNPSLKS | 5039 | ERGYDSEYYFYYYMDV |
| 07D03 | 4724 | SSNWWS | 4882 | EIYHSGSTSYNPSLKS | 5040 | ERGYSSPHYYFYYYMDV |
| 07G08 | 4725 | YSTMT | 4883 | SIGGIGGYTYYADSVKG | 5041 | ERVAANYFDY |
| 07G01 | 4726 | SSNWWS | 4884 | EIYHSGSTSYNPSLKS | 5042 | ESGYDDDYYFYYYMDV |
| 04B07 | 4727 | NYGIS | 4885 | WISAYNGNTKYAQKLQG | 5043 | ESSSWYGPLFAI |
| 04C03 | 4728 | SSDLWS | 4886 | EIYHSGTTNSNPSLKS | 5044 | EVYSSSSGGYWYFDL |
| 05D03 | 4729 | SNNWWS | 4887 | EIYHTGSTNYNPSLKS | 5045 | FVVAPDPIGYFDP |
| 05G09 | 4730 | SSNWWS | 4888 | EIYHSGSTNYNPSLKS | 5046 | FVVAPVPIGYFDP |
| 02F02 | 4731 | SSDWWS | 4889 | EVYHSGSTNYNPSLRS | 5047 | FVVEPDLIGWFDP |
| 12E03 | 4732 | SSNWWS | 4890 | EIYHSGSTNYNPSLKS | 5048 | GAAVDGAGEDY |
| 10F02 | 4733 | SSNWWT | 4891 | EIYHSGSSNYNPSLKS | 5049 | GATGDGAGFDY |
| 10A03 | 4734 | SSNWWS | 4892 | EIYHSGSTNYNPSLKS | 5050 | GATVDGAGFDY |
| 05F11 | 4735 | SNSAAWN | 4893 | RTYFRSEWYTDYAVSVKS | 5051 | GDIAAAGYYFDY |
| 04E01 | 4736 | TNSAAWN | 4894 | RTYYRSRWYNDYAVSVKS | 5052 | GDIGAAGYYFDY |
| 04A08 | 4737 | SNSAAWN | 4895 | RTYYRSKWYNDYGVSVTG | 5053 | GDIGTAGFYFDY |
| 04E03 | 4738 | TNGASWN | 4896 | RTYYRSDWYNDYTGSVIG | 5054 | GDIGTAGYYFDS |
| 04H07 | 4739 | TNSASWN | 4897 | RTYYRSNWYTDYAVSVIS | 5055 | GDVGSAGYFFDY |
| 08F01 | 4740 | TSLMN | 4898 | SITSGGTYISYTDSVKG | 5056 | GDWNYPPYYYYMDV |
| 04G03 | 4741 | RNSVAWN | 4899 | RTDYRSMWSGDYVESVQG | 5057 | GEAGSAGFYFDY |
| 02A09 | 4742 | RNSSAWN | 4900 | RTYYWSKWYYEYAISVKG | 5058 | GEAGTLDY |
| 04F08 | 4743 | SNSAAWN | 4901 | RTYYRSNWFGDNAASVQG | 5059 | GETGTAGYYFDS |
| 01E11 | 4744 | SISASWN | 4902 | RTYYRSKWYNDYGVSVKS | 5060 | GETGTAGYYFDY |
| 04F09 | 4745 | SNSAAWN | 4903 | RTYYRSNWYGDYAVSVQG | 5061 | GETGTAGYYFDY |
| 04E05 | 4746 | SKSASWN | 4904 | RTYYRSEWYNDYALSVIS | 5062 | GETGTAGYYMDV |
| 04C02 | 4747 | NNSAAWN | 4905 | RTYYRSEWENDYGASVIG | 5063 | GEVGASGYYMDV |
| 01E02 | 4748 | SNGATWN | 4906 | RTYYRSQWYNDYGVSVLS | 5064 | GGIAGAGTLFDY |
| 01H04 | 4749 | SNSATWN | 4907 | RTYYRSKWYNDYSVSVTS | 5065 | GGIASAGTFFDY |
| 04C10 | 4750 | NNSASWN | 4908 | RTFYRSNWYNDYSVSVIS | 5066 | GGIGAAGYYFDY |
| 08E10 | 4751 | GYYMH | 4909 | WINPNSGGTNYAQKFQG | 5067 | GGVCSGITCYGYGSDY |
| 07C03 | 4752 | GNYMH | 4910 | WINPNSGGTNYVQKFQG | 5068 | GGVCSGTTCYGYGSDY |
| 04H11 | 4753 | HNSASWN | 4911 | RTYYRSSWYNDYAVSVIS | 5069 | GGVGPAGYYFDY |
| 03D07 | 4754 | GYYMH | 4912 | WINPKSDGTNYAQKFQG | 5070 | GGVYGSRWPKAFDI |
| 05C08 | 4755 | TYSMN | 4913 | YISSSSSTIYYADSVKG | 5071 | GGYDTSGFAFDI |
| 03C05 | 4756 | GYYMH | 4914 | WINPTSGATNYAQKFQG | 5072 | GHLLLPDAFDI |
| 03H05 | 4757 | GYYMH | 4915 | WINPTSGATNYAQKFQG | 5073 | GHLLLPDAFDI |
| 01D10 | 4758 | GNYIH | 4916 | WINPNSGVTYSAQKLQG | 5074 | GHQLVPDAFDI |
| 07B02 | 4759 | GYDMH | 4917 | VIWYDGSNKYYADSVKG | 5075 | GIAGAAFDY |
| 08F03 | 4760 | SNSAAWN | 4918 | RTYYRSRWYNDYAVSVKS | 5076 | GKEWGHTNTWYGVNYYYMDV |
| 10E11 | 4761 | SSNWWN | 4919 | EIYHSGSTSYNPSLKS | 5077 | GKGYDLEYYYYYYMDV |
| 04F12 | 4762 | NYYIH | 4920 | WINSNSGVTNYAQKFQD | 5078 | GKWELPDAFDF |
| 04D01 | 4763 | NNGASWN | 4921 | RTYYRSGWYNDYAVSVIS | 5079 | GNIGPAGYHFDY |
| 04F04 | 4764 | SNSAAWN | 4922 | RTYYRSDWYNDYGISVTS | 5080 | GNVGPAGYHFDY |
| 07D01 | 4765 | GYYWS | 4923 | DINHSGSINYNPSLKS | 5081 | GPAGTDAFDI |
| 10D04 | 4766 | SSNWWN | 4924 | EIYHSGSTNYNPSLKS | 5082 | GPSRDGAGFDY |
| 10C01 | 4767 | SSNWWN | 4925 | EIYHSGSTNYNPSLKS | 5083 | GPSVDGAGFDY |
| 10E01 | 4768 | SSNWWN | 4926 | EIYHSGSTNYNPSLKS | 5084 | GPTGDGAGEDY |
| 10F07 | 4769 | TYSMN | 4927 | SISSSGFYIYYADSVKG | 5085 | GPTGNYYYFMDV |
| 03B10 | 4770 | GYYMH | 4928 | WINPNSGVTNYSQKFQG | 5086 | GQQLVPDAFDI |
| 06E02 | 4771 | GYYIH | 4929 | WINPISGDTHFAQKFQG | 5087 | GSTWPDVFGI |
| 04F03 | 4772 | NNSAAWN | 4930 | RTYYRSKWYNDYAKS | 5088 | GTIAAAGYYFDY |
| 08G03 | 4773 | NYGIS | 4931 | WIRTYNGNTNYAQRFQG | 5089 | GTLFNWIDP |
| 04H03 | 4774 | SNSASWN | 4932 | RTYYRSNYYNDYAVSVPS | 5090 | GVVGASGYYMDV |
| 06G05 | 4775 | GHYIH | 4933 | WSNPYSGATHYAEKFQG | 5091 | GYNWNPDAFDI |
| 12B10 | 4776 | SSYYYWG | 4934 | SFYYNGFTYYNPSLKS | 5092 | HEMGANAFDI |
| 11G03 | 4777 | SSYYYWG | 4935 | SFYYNGFTYYNPSLKS | 5093 | HEVGANAFDI |
| 04B12 | 4778 | SSNYYWG | 4936 | NIYFRGNTYYNPSLKS | 5094 | HGYDSFDY |
| 10H10 | 4779 | SSSYYWG | 4937 | NIFYSGNTYYNPSLKS | 5095 | HYFSSSFDY |
| 05G01 | 4780 | SYVVS | 4938 | WITPYNGNTNYAQNLQD | 5096 | KGPTNYYSYMDV |
| 10H01 | 4781 | GHDMH | 4939 | VIWYDGSNKYYGDSVKG | 5097 | KTQFGFDY |
| 11B03 | 4782 | NYAMH | 4940 | VIWYDGSNKYYADSVKG | 5098 | LAADAPKWEDP |
| 08H01 | 4783 | SSSYYGG | 4941 | SIYYSGSTYYNPSLKS | 5099 | LYNWNYGGGFFDY |
| 05D06 | 4784 | SGGYYWS | 4942 | YIHHRGTTYFNPSLKS | 5100 | MVRGIPLYYYFMDV |
| 05D11 | 4785 | SGVYYWS | 4943 | YISNSETTYYNPSLKS | 5101 | MVRGLDYYYYYMDV |
| 05C06 | 4786 | SGDYYWS | 4944 | YIYYRGTTYYNPSLKS | 5102 | MVRGLKYYYYYMDV |
| 05H02 | 4787 | SGDYYWS | 4945 | YIYYRGTTYYNPSLKS | 5103 | MVRGLNYYYYHMDV |
| 05E03 | 4788 | SGDYYWS | 4946 | YISYIGTTYYNPSLKS | 5104 | MVRGLNYYYYMDV |
| 05D01 | 4789 | SGDYYWS | 4947 | YIYYRGTTFYNPSLKS | 5105 | MVRGLNYYYYYMDA |
| 05B04 | 4790 | SGDYYWS | 4948 | YIYYRGTTYYNPSLKS | 5106 | MVRGLNYYYYYMDV |
| 05E01 | 4791 | SGDYYWS | 4949 | YIYYRGTTYYNPSLKS | 5107 | MVRGLNYYYYYMDV |
| 05G03 | 4792 | SNGYYWT | 4950 | YMSYIGTTYSNPSLKS | 5108 | MVRGLTYYYYMDV |
| 05C01 | 4793 | SGVYYWS | 4951 | YISYSGTTYYNPSLKS | 5109 | MVRGVDYYYYYMDV |
| 05E07 | 4794 | SGVYYWS | 4952 | YISNSGTTYYNPSLKS | 5110 | MVRGVNYYYYMDV |
| 05B02 | 4795 | SGVYYWS | 4953 | YISNSGTTYYNPSLKS | 5111 | MVRGVNYYYYYMDV |
| 06C02 | 4796 | RTNYYWA | 4954 | NIYYSGDTYYNPSLKS | 5112 | NGYDSFDY |
| 05C04 | 4797 | TYSMN | 4955 | SIGGSSSYTYYADSVKG | 5113 | QGYYDSSGYYFPDAFDI |
| 03G04 | 4798 | GYYWS | 4956 | EINHYGSTNYNPSLKS | 5114 | QNWNYEGFDY |
| 09B05 | 4799 | SYWIG | 4957 | IIYPGDSATRYSPSFQG | 5115 | QPLNYDWFDP |
| 02D06 | 4800 | SFGFS | 4958 | WTSGYNGITKYSQMFQD | 5116 | RDNWNYDAFDL |
| 08A03 | 4801 | SSTFYWG | 4959 | SIYYSGNTYYNPSLKS | 5117 | RDWNHIDY |
| 06G08 | 4802 | TSGEGVG | 4960 | LIYWNDDKRYSPSLKN | 5118 | RGFKSRWYVDY |
| 05B01 | 4803 | TSGEGVG | 4961 | LIYWNDDKRYNPSLEN | 5119 | RGFRSRWYVDF |
| 10A01 | 4804 | STGFFWG | 4962 | SIYYSGNTYYNPSLKS | 5120 | RSPRWSSSGSDAFDI |
| 02G05 | 4805 | SYGIS | 4963 | WISAYDGDTNYAQKFQG | 5121 | RWNYEKGAFDI |
| 02E01 | 4806 | SYGIS | 4964 | WTSGYNGITKYAQKFQD | 5122 | RYNWNYAAFDL |
| 05C03 | 4807 | NYGVS | 4965 | WIGANNAYTKYAQKLQG | 5123 | RYNWNYDAFDI |
| 08E05 | 4808 | NYGVS | 4966 | WISAYNGDTNYAQKFQG | 5124 | RYNWNYGIFDN |
| 08H07 | 4809 | NYGIS | 4967 | WISAYNGNTNYAQKFQG | 5125 | RYNWNYGILDY |
| 08B04 | 4810 | SYGIT | 4968 | WISVYNGDTNYAQNLQG | 5126 | RYNWNYGLEDY |
| 11F01 | 4811 | NYGIS | 4969 | WISAYNGDTNYAQKIQG | 5127 | RYNWNYGLLDY |
| 02E06 | 4812 | SFGIN | 4970 | WTSGYNGITKYVEKFQD | 5128 | RYNWNYVAFDL |
| 10C02 | 4813 | NSNWWT | 4971 | EIYHSVNTNYNPSLKS | 5129 | SPYGGNPYFDY |
| 03G02 | 4814 | GYYIH | 4972 | WINPYSGATNYAQKFQG | 5130 | SRLPDAFDI |
| 06F09 | 4815 | NLGIS | 4973 | WISPYNGFTNYAQKLQG | 5131 | TFPLDY |
| 06G02 | 4816 | NYGIS | 4974 | WISAYNGNTNYAQKFQG | 5132 | TWNSDY |
| 06D01 | 4817 | NYGIS | 4975 | WISVYNGYTKYSQKLQG | 5133 | TYAFDI |
| 07D10 | 4818 | SYNMH | 4976 | IIHPSGGSTNYAQKFQG | 5134 | VGDYFDY |
| 02E03 | 4819 | TYGIS | 4977 | WISAYYGNTNYAQKLQG | 5135 | VRYSSSWGWYFDL |
| 05C05 | 4820 | SNSAAWN | 4978 | RTYYMSKWFTEYAVSVKR | 5136 | WDLGSLDY |
| 05D05 | 4821 | SNNWWS | 4979 | EIYHGGSTNYNPSLKS | 5137 | WNIELFRGYFDY |
| 05A06 | 4822 | GSDWWS | 4980 | EIFHSGSTNYNPSLKS | 5138 | WNIENFAGYFDL |
| 05H01 | 4823 | SSNWWS | 4981 | EIYHGGSTSYNPSLKS | 5139 | WNITLFRGYFDY |
| 02D01 | 4824 | SSNWWS | 4982 | EIYHSGSTNYNPSLRS | 5140 | WNIVGALGYFDL |
| 10G05 | 4825 | STNWWN | 4983 | EIYHSGTTNHNPSLKS | 5141 | YYYGSGSYAFDI |

TABLE 19
VL-CDR1, VL-CDR2, VL-CDR3 Sequences for EPOR/CD131 Binders IV
(Phage Library) of Table 17

| Clone | SEQ | | SEQ | CDRL2 | SEQ | |
|---|---|---|---|---|---|---|
| ID | ID NO | CDRL1 Sequence | ID NO | Sequence | ID NO | CDRL3 Sequence |
| 06C01 | 5142 | RASQGIRGWLA | 5300 | SASSLQS | 5458 | QQSYSTPLT |
| 06H04 | 5143 | RASQSISSWLA | 5301 | KASSLES | 5459 | QQSYSSPLT |
| 10H09 | 5144 | TGSSSNIGAGYDVH | 5302 | GNSNRPS | 5460 | QSYDSSLSGWV |
| 03G03 | 5145 | RASQGIRNDLG | 5303 | AASSLQS | 5461 | LQDYNYPFT |
| 11F05 | 5146 | TGSSSNIGAGYDVH | 5304 | GNSNRPS | 5462 | QSYDSSLSGWV |
| 07H07 | 5147 | TGSSSNIGAGYDVH | 5305 | GNSNRPS | 5463 | QSYDSRLSGNWV |
| 08D01 | 5148 | TGSSSNIGAGYDVH | 5306 | GNSNRPS | 5464 | QSYDSSLSGYV |
| 11B05 | 5149 | TGSSSSIGAGYDVH | 5307 | GNSNRPS | 5465 | QSYDSSLSGWV |
| 11A03 | 5150 | TGSSSNIGAGYDVH | 5308 | GNSNRPS | 5466 | QSYDSSLSGWV |
| 11H08 | 5151 | TGSSSNIVAGYDVH | 5309 | GNTNRPS | 5467 | QSYDSSLSGWV |
| 10F01 | 5152 | TGSSSNIGAGYDVH | 5310 | GNSNRPS | 5468 | QSYDSSLSGSV |
| 08C09 | 5153 | TGSSSNIGAGYDVH | 5311 | GNSNRPS | 5469 | QSYDSSLSGWV |
| 10F08 | 5154 | TGSSSNIGAGYDVH | 5312 | GNSNRPS | 5470 | QSYDSSLSGWV |
| 11F06 | 5155 | TGSSSNIGAGYDVH | 5313 | GNSNRPS | 5471 | QSYDSSLSGWV |
| 05H05 | 5156 | RSSQSLVSSVGNTYLN | 5314 | KVSDRDS | 5472 | MQGTHWPPT |
| 06A09 | 5157 | RASQGIGSYLA | 5315 | AASTLQS | 5473 | QQLNSYPLT |
| 01E06 | 5158 | RASQGISSYLA | 5316 | AASTLQS | 5474 | QQFHSYPFT |
| 02F06 | 5159 | RSSQSLVYSYGNTYLN | 5317 | KVSNRDS | 5475 | MQGTYWPPT |
| 04B04 | 5160 | RASQGISSYLA | 5318 | AASSLQG | 5476 | LQDYNYPLT |
| 04D11 | 5161 | RASQGISSYLA | 5319 | AASTLQS | 5477 | QQLNSYPIT |
| 04D07 | 5162 | RASQGISSYLA | 5320 | AASTLQS | 5478 | QQLNSYPLT |
| 06C09 | 5163 | RASQSVSSYLA | 5321 | DASNRAT | 5479 | QQRSNWPYT |
| 08F05 | 5164 | SGDKLGSKYAF | 5322 | QDNKRPS | 5480 | QAWDGSSAV |
| 10F04 | 5165 | GGNNIGSENVY | 5323 | RDSNRPS | 5481 | QVWDSSTVV |
| 09F01 | 5166 | SGDALPKKYAY | 5324 | EDSKRPS | 5482 | YSTDSSGNHYV |
| 05H09 | 5167 | RSSQSLVYSDGNTYLN | 5325 | KVSNRDS | 5483 | MQGTHWPRT |
| 06C12 | 5168 | RSSQSLVYSDGNTYLN | 5326 | KVSNRDS | 5484 | MQGTHWPRT |
| 07C09 | 5169 | TGTSSDVGGYNYVS | 5327 | EVSNRPS | 5485 | SSYTSSSTLVV |
| 07E03 | 5170 | TGTSSDVGGYNYVS | 5328 | EVSNRPS | 5486 | SSYTSSSTLV |
| 02H08 | 5171 | RSSQSLLHSNGYNYLD | 5329 | LGSNRAS | 5487 | MQGLQTPWT |
| 06D02 | 5172 | RSSRSLVYSDGNTYLN | 5330 | KVSNRDS | 5488 | MQGTNWPPYT |
| 07E04 | 5173 | SGSSSNIGRNPVY | 5331 | DNNQRPS | 5489 | AAWDDSLEV |
| 02C03 | 5174 | RASQGIAGWLA | 5332 | SASSLQS | 5490 | QQANSFPRT |
| 08F02 | 5175 | TGSSSNIGAGYDVH | 5333 | GNSNRPS | 5491 | QSYDSSLSGSMV |
| 06F01 | 5176 | RASQSISNWLA | 5334 | KASSLQS | 5492 | QQYKSFIT |
| 10A04 | 5177 | SGDKLGDKYVS | 5335 | QDSERPS | 5493 | QAWDSSTVV |
| 07C08 | 5178 | SGSSSNIGSNTVN | 5336 | SNNQRPS | 5494 | AAWDDSLNGVV |
| 04B08 | 5179 | RASQSISSYLN | 5337 | AASSLQS | 5495 | QQSYSTPLT |
| 07D05 | 5180 | GGNNIGSKSVH | 5338 | YDSDRPS | 5496 | QVWDSSSDHVV |
| 03C12 | 5181 | RASQSISSYLN | 5339 | AASSLQS | 5497 | QQSYSTPIT |
| 07G09 | 5182 | SGSSSNIGSNTVN | 5340 | SNNQRPS | 5498 | AAWDDSLNGYV |
| 07H01 | 5183 | SGSSSNIGSNTVN | 5341 | SNNQRPS | 5499 | AAWDDSLNGYV |
| 07B01 | 5184 | TGTSSDVGGYNYVS | 5342 | DVSKRPS | 5500 | SSYTSSSTLV |
| 07A01 | 5185 | TGSSSNIGAGYDVH | 5343 | GNSNRPS | 5501 | QSYDSSLSGWV |
| 09F09 | 5186 | SGDALPKKYAY | 5344 | EDSKRPS | 5502 | YSTDSSGNHRV |
| 07C01 | 5187 | TGSSSNIGSGYDVH | 5345 | GNSNRPS | 5503 | QSYDSSLSGWV |
| 06G01 | 5188 | RASQGIRNDLG | 5346 | AASTLQS | 5504 | QQLNSYPIT |
| 06A01 | 5189 | RASQGISSYLA | 5347 | AASTLQS | 5505 | QQLNSYPFT |
| 06B06 | 5190 | RASQGISSYLA | 5348 | AASTLQS | 5506 | QQLNSYPHT |
| 06A02 | 5191 | RASQGIRNDLG | 5349 | AASSLQS | 5507 | LQLNSYPLT |
| 06F12 | 5192 | RASQSVSRYLA | 5350 | DASNRAT | 5508 | QQRSNWPPIT |
| 05D12 | 5193 | RSSQSLVYSDGNTYLN | 5351 | KVSNRDS | 5509 | MQGTHWPFT |
| 05D07 | 5194 | RSSQSLVYSDGNTYLN | 5352 | KVSNRDS | 5510 | MQGTHWPFT |
| 02F04 | 5195 | RASQSVSSNLA | 5353 | GASTRAT | 5511 | QQRSNWPLT |
| 02A07 | 5196 | RASQSVHKYLA | 5354 | DASSRAT | 5512 | QQRSNWPLT |
| 07C02 | 5197 | TGSSSNIGAGYDVH | 5355 | GNSNRPS | 5513 | QSYDSSLSGYV |
| 07D03 | 5198 | TGSSSNIGAGYDVH | 5356 | GNSNRPS | 5514 | QSYDSSLSGYV |
| 07G08 | 5199 | SGDKLGNKYAS | 5357 | QDRKRPS | 5515 | QAWDSSTYVI |
| 07G01 | 5200 | TGSSSNIGAGYDVH | 5358 | GNGNRPS | 5516 | QSYDSSLSGYV |
| 04B07 | 5201 | RASQGISSYLA | 5359 | AASTLQS | 5517 | QQLNSYPLT |
| 04C03 | 5202 | RASQSISSYLN | 5360 | AASSLQS | 5518 | QQSYSTPFT |
| 05D03 | 5203 | RAGQSISSYLN | 5361 | AASSLQS | 5519 | QQSYSTPFT |
| 05G09 | 5204 | RASQSISSYLN | 5362 | AASSLQS | 5520 | QQSYSTPFT |
| 02F02 | 5205 | RASQSISSYLN | 5363 | AASSLQG | 5521 | QQSYSTPLT |
| 12E03 | 5206 | SGDKLGNKYAS | 5364 | QDSKRPS | 5522 | QAWDSSTAVV |
| 10F02 | 5207 | SGDKLGDKYVS | 5365 | QDRKRPS | 5523 | QAWDSSTAVV |
| 10A03 | 5208 | SGDKLGYKYAS | 5366 | QDRKRPS | 5524 | QAWDSSTAVV |
| 05F11 | 5209 | RASQSISSYLN | 5367 | AASSLQS | 5525 | QQSYSTPLT |
| 04E01 | 5210 | RASQSISSYLN | 5368 | AASSLQS | 5526 | QQSYSPPLT |
| 04A08 | 5211 | RASQSIGSYLN | 5369 | GASGLQS | 5527 | QQSFSTPFT |
| 04E03 | 5212 | RASRNIGTYLN | 5370 | AASSLQS | 5528 | QQSFSTPYT |
| 04H07 | 5213 | RASQSISNYLN | 5371 | AASSLQS | 5529 | QQTYSTPLT |
| 08F01 | 5214 | TGTSSDVGGYNYVS | 5372 | EVSNRPS | 5530 | SSYTSSSTVV |
| 04G03 | 5215 | RASQSISSYLN | 5373 | AASSLQS | 5531 | QQSYSALT |
| 02A09 | 5216 | RSSQSLVYSDGNTYLN | 5374 | EVSNRDS | 5532 | MQGTHWPYT |
| 04F08 | 5217 | RASQTIISYLN | 5375 | AASSLHS | 5533 | QQSYSTPIT |
| 01E11 | 5218 | RASQSISSYLN | 5376 | AASSLQG | 5534 | QQSYSTPLT |
| 04F09 | 5219 | RASQSISSYLN | 5377 | AASSLQS | 5535 | QQSYSPPLT |
| 04E05 | 5220 | RASQSISSYLN | 5378 | AASSLOS | 5536 | QQSYSTPLT |
| 04C02 | 5221 | RASQSISSYLN | 5379 | AASSLQS | 5537 | QQSYSTPLT |
| 01E02 | 5222 | RASQSISSYLN | 5380 | AASSLQS | 5538 | QQSYSTPLT |
| 01H04 | 5223 | RASQSISSYLN | 5381 | AASSLQT | 5539 | QQSYSTPLT |
| 04C10 | 5224 | RASQSISTYLN | 5382 | AASSLQS | 5540 | QQSYSTPIT |
| 08E10 | 5225 | TGSSSNIGAGYDVH | 5383 | GNSNRPS | 5541 | QSYDSSLSGWV |
| 07C03 | 5226 | TGSSSNIGAGYDVH | 5384 | GNSNRPS | 5542 | QSYDSSLSGWV |
| 04H11 | 5227 | RASQSISSYLN | 5385 | AASSLQS | 5543 | QQTYSTPLT |
| 03D07 | 5228 | RASQSISTYLN | 5386 | AASSLQS | 5544 | QQSYSTPLT |
| 05C08 | 5229 | RASQGISNYLA | 5387 | AASTLQS | 5545 | QQANSFPPT |
| 03C05 | 5230 | RASQSISSYLN | 5388 | GASRLQS | 5546 | QQSYSTPIT |
| 03H05 | 5231 | RASQSISSYLN | 5389 | GASRLQS | 5547 | QQSYSTPIT |
| 01D10 | 5232 | RASQNISSWLD | 5390 | AASRLQS | 5548 | QQSYSTPLT |
| 07B02 | 5233 | TGTSSDVGGYNYVS | 5391 | DVSKRPS | 5549 | LSYDSSLSGSV |
| 08F03 | 5234 | TGSSSNIGAGYDVH | 5392 | GNSNRPS | 5550 | QSYDSSLSGWV |
| 10E11 | 5235 | TGSSSNIGAGYDVH | 5393 | GNSNRPS | 5551 | QSYDSSLSGSV |
| 04F12 | 5236 | RASQGISSWLA | 5394 | AASRLQS | 5552 | QQSYSTPLT |
| 04D01 | 5237 | RASQTISSYLN | 5395 | AASSLQS | 5553 | QQSYGTPLT |
| 04F04 | 5238 | RASQSISSNLN | 5396 | AASNLQT | 5554 | QQSYGLPLT |
| 07D01 | 5239 | TGTSSDVGGYNYVS | 5397 | EVNKRPS | 5555 | SSYAGSNNLV |
| 10D04 | 5240 | SGDKLGNKYAS | 5398 | QDSKRPS | 5556 | QAWDSSTAVV |
| 10C01 | 5241 | SGDKLGDKYAS | 5399 | QDSKRPS | 5557 | QAWDSSTAVV |
| 10E01 | 5242 | SGDKLGNKYVS | 5400 | QDSKRPS | 5558 | QAWDSSTAVV |
| 10F07 | 5243 | GGNNIGSKSVN | 5401 | YDSDRPS | 5559 | QVWDSSSDHVV |
| 03B10 | 5244 | RASQGISRWLD | 5402 | AASSLQS | 5560 | QQSYSTPLT |
| 06E02 | 5245 | RASQAIRRWLA | 5403 | SASSLHS | 5561 | QQSYSTPLT |
| 04F03 | 5246 | RASQSISSYLN | 5404 | AASSLQS | 5562 | QQSYSTPLT |
| 08G03 | 5247 | TGTSSDVGGYNYVS | 5405 | DVSKRPS | 5563 | SSYAGRNNLV |
| 04H03 | 5248 | RASQSISRYLN | 5406 | AASSLQS | 5564 | QQSYSTPLT |
| 06G05 | 5249 | RASQSIRSYLN | 5407 | GASSLQR | 5565 | QQSYSSPLT |
| 12B10 | 5250 | SGDALPKKYAY | 5408 | EDSKRPS | 5566 | YSTDSSGNHRV |
| 11G03 | 5251 | SGDALPKKYAY | 5409 | EDSKRPS | 5567 | YSTDSSGNHRV |
| 04B12 | 5252 | RASQSVSKYLA | 5410 | DASNRAT | 5568 | QQRSNWPLT |
| 10H10 | 5253 | TGTSSDVGGYNYVS | 5411 | EVSNRPS | 5569 | CSYEGSWV |
| 05G01 | 5254 | RASQSVSSSYLA | 5412 | GASSRAT | 5570 | QQYGSSPLT |
| 10H01 | 5255 | TGSSSKIGAGYDVH | 5413 | GNSNRPS | 5571 | QSYDSSLSGSV |
| 11B03 | 5256 | QGDSLRNYYAS | 5414 | GNNNRPS | 5572 | NSRDSSGNHVV |
| 08H01 | 5257 | TGSSSNIGAGYDVH | 5415 | GNSNRPS | 5573 | QSYDSSLSGWV |
| 05D06 | 5258 | RASQGLRNWLA | 5416 | AASGLQG | 5574 | QQANIFPLT |
| 05D11 | 5259 | RASQGISSWLV | 5417 | AASSLQS | 5575 | QQANSFPLT |
| 05C06 | 5260 | RASQGISSWLA | 5418 | AASSLQS | 5576 | QQANSFPLT |
| 05H02 | 5261 | RASQGISSWLA | 5419 | AASSLQS | 5577 | QQANSFPLT |
| 05E03 | 5262 | RASQGISSWLA | 5420 | AASSLQS | 5578 | QQANSFPIT |
| 05D01 | 5263 | RASQGISSWLA | 5421 | AASSLQS | 5579 | QQANSFPPT |
| 05B04 | 5264 | RASQGISSWLA | 5422 | AASSLQS | 5580 | QQANSFPPT |
| 05E01 | 5265 | RASQGISSWLA | 5423 | AASSLQS | 5581 | QQANSFPPT |
| 05G03 | 5266 | RASQGLSNWLA | 5424 | AASSLQS | 5582 | QQANGFPIT |
| 05C01 | 5267 | RASQGISRWLA | 5425 | AASSLQS | 5583 | QQANSFPLT |
| 05E07 | 5268 | RASQGLSNWLA | 5426 | AASGLQG | 5584 | QQANIFPLT |
| 05B02 | 5269 | RASQGISSWLA | 5427 | AASSLQS | 5585 | QQANSFPLT |
| 06C02 | 5270 | RASQSVSSYLA | 5428 | DASNRAT | 5586 | QQRSNWPLT |
| 05C04 | 5271 | RSSQSLVYSDGNTYLN | 5429 | KVSNRDS | 5587 | MQGTHWPFT |
| 03G04 | 5272 | RASQGIRNDLG | 5430 | AASSLQS | 5588 | LQDYNYPYT |
| 09B05 | 5273 | TGTSSDVGGYNYVS | 5431 | DVSKRSS | 5589 | CSYAGSSTWV |
| 02D06 | 5274 | RASQSISSYLN | 5432 | AASSLQS | 5590 | QQAYSFPYT |
| 08A03 | 5275 | SGDALPKKYAY | 5433 | EDSKRPS | 5591 | YSTDSSGNHYV |
| 06G08 | 5276 | RASQSIGSYLA | 5434 | DASNRAT | 5592 | QQRSNWPFT |
| 05B01 | 5277 | RASQSVSSYLA | 5435 | DASNRAT | 5593 | QQRSNWPFT |
| 10A01 | 5278 | SGSSSNIGSNTVN | 5436 | SNNQRPS | 5594 | AAWDDSLNGWV |
| 02G05 | 5279 | RASQISSRHLN | 5437 | AASSLQG | 5595 | QQSYSTPFT |
| 02E01 | 5280 | RASQSISSYLN | 5438 | AASSLQS | 5596 | QQSYSTPLT |
| 05C03 | 5281 | RASQSISSYLN | 5439 | AASSLQS | 5597 | QQSYSTPLT |
| 08E05 | 5282 | QGDSLRSYYAS | 5440 | GKNNRPS | 5598 | NSRDSSGNHVV |
| 08H07 | 5283 | QGDSLRSYYAS | 5441 | GKNNRPS | 5599 | NSRDSSGNHVV |
| 08B04 | 5284 | QGDSLRSYYAS | 5442 | GKNNRPS | 5600 | NSRDSSGNHLV |
| 11F01 | 5285 | QGDSLRSYYAS | 5443 | GKNNRPS | 5601 | NSRDSSGNHVV |
| 02E06 | 5286 | RASQSISSYLN | 5444 | AASSLQS | 5602 | QQSYSTPLT |
| 10C02 | 5287 | SGDKLGDKYAS | 5445 | QDNKRPS | 5603 | QAWDSSTVV |
| 03G02 | 5288 | RASQNINSYLN | 5446 | AASSLQS | 5604 | QQSYSTPIT |
| 06F09 | 5289 | RASQSISSYLN | 5447 | AASSLQS | 5605 | LQDSNYPLT |
| 06G02 | 5290 | RASQSISSYFN | 5448 | AASSLQS | 5606 | QQTDSSPWT |
| 06D01 | 5291 | RASQSISSYLN | 5449 | AASSSQS | 5607 | QQSYSTPFT |
| 07D10 | 5292 | TGTSSDVGGYNYVS | 5450 | DVSKRPS | 5608 | CSYAGSYTWV |
| 02E03 | 5293 | RASQSISSYLN | 5451 | AASSLQS | 5609 | QQSYSTPLT |
| 05C05 | 5294 | RSSQSLVYSDGNTYLN | 5452 | KVSNRDS | 5610 | MQGTHWPYT |
| 05D05 | 5295 | RASQSTSSYLN | 5453 | AASSLQS | 5611 | QQSYSTPFT |
| 05A06 | 5296 | RASQSISSYLN | 5454 | AASSLQS | 5612 | QQSFSTPFT |
| 05H01 | 5297 | RASQSISSYLN | 5455 | AASSLQS | 5613 | QQSYSTPFT |
| 02D01 | 5298 | RASQSISSYLN | 5456 | AASSLQS | 5614 | QQSYSTPFT |
| 10G05 | 5299 | TGTSSDVGGYNYVS | 5457 | EVSNRPS | 5615 | SSYTSSSTWV |

TABLE 20
VH and VL Sequences for EPOR Binders (germline antibodies)

| Clone | SEQ | | SEQ | |
|---|---|---|---|---|
| ID | ID NO | Heavy Chain | ID NO | Light Chain |
| A1- | 5616 | MGWSCIILFLVATATGVHSQVQLQQSGPGLVKP | 5804 | MGWSCIILFLVATATGV |
| LALA | | SQTLSLTCAISGDSVSSNSAAWNWIRQSPSRGL | | HSQSALTQPASVSGSPG |
| PGhL | | EWLGRTYYRSKWYNDYAVSVKSRITINPDTSKN | | QSITISCTGTSSDVGGY |
| 2 | | QFSLQLNSVTPEDTAVYYCARKGELLGAFDIWG | | NYVSWYQQHPGKAPKLM |
| | | QGTMVTVSSASTKGPSVFPLAPSSKSTSGGTAA | | IYEVSNRPSGVSNRFSG |
| | | LGCLVKDYFPEPVTVSWNSGALTSGVHTFPAVL | | SKSGNTASLTISGLQAE |
| | | QSSGLYSLSSVVTVPSSSLGTQTYICNVNHKPS | | DEADYYCSSYTSSSTWV |
| | | NTKVDKKVEPKSCDKTHTCPPCPAPEAAGGPSV | | FGGGTKLTVLGQPKAAP |
| | | FLFPPKPKDTLMISRTPEVTCVVVDVSHEDPEV | | SVTLFPPSSEELQANKA |
| | | KFNWYVDGVEVHNAKTKPREEQYNSTYRVVSVL | | TLVCLISDFYPGAVTVA |
| | | TVLHQDWLNGKEYKCKVSNKALGAPIEKTISKA | | WKADSSPVKAGVETTTP |
| | | KGQPREPQVYTLPPSREEMTKNQVSLTCLVKGF | | SKQSNNKYAASSYLSLT |
| | | YPSDIAVEWESNGQPENNYKTTPPVLDSDGSFF | | PEQWKSHRSYSCQVTHE |
| | | LYSKLTVDKSRWQQGNVFSCSVMHEALHNHYTQ | | GSTVEKTVAPTECS |
| | | KSLSLSPGK | | |
| A2- | 5617 | MGWSCIILFLVATATGVHSQVQLQQSGPGLVKP | 5805 | MGWSCIILFLVATATGV |
| LALA | | SQTLSLTCAISGDSVSSNSAAWNWIRQSPSRGL | | HSQSALTQPASVSGSPG |
| PGhL | | EWLGRTYYRSKWYNDYAGSVKSRIIIIPDTSKN | | QSITISCTGTSSDVGGY |
| 2 | | QLSLQLKSVTPEDTAVYYCARKWELRDAFDIWG | | NYVSWYQQHPGKAPKLM |
| | | QGTMVTVSSASTKGPSVFPLAPSSKSTSGGTAA | | IYDVSKRPSGVSNRFSG |
| | | LGCLVKDYFPEPVTVSWNSGALTSGVHTFPAVL | | SKSGNTASLTISGLQAE |
| | | QSSGLYSLSSVVTVPSSSLGTQTYICNVNHKPS | | DEADYYCCSYAGRSTLG |
| | | NTKVDKKVEPKSCDKTHTCPPCPAPEAAGGPSV | | IDWVFGGGTKVTVLGQP |
| | | FLFPPKPKDTLMISRTPEVTCVVVDVSHEDPEV | | KAAPSVTLFPPSSEELQ |
| | | KFNWYVDGVEVHNAKTKPREEQYNSTYRVVSVL | | ANKATLVCLISDFYPGA |
| | | TVLHQDWLNGKEYKCKVSNKALGAPIEKTISKA | | VTVAWKADSSPVKAGVE |
| | | KGQPREPQVYTLPPSREEMTKNQVSLTCLVKGF | | TTTPSKQSNNKYAASSY |
| | | YPSDIAVEWESNGQPENNYKTTPPVLDSDGSFF | | LSLTPEQWKSHRSYSCQ |
| | | LYSKLTVDKSRWQQGNVFSCSVMHEALHNHYTQ | | VTHEGSTVEKTVAPTEC |
| | | KSLSLSPGK | | S |
| A3- | 5618 | MGWSCIILFLVATATGVHSEVQLVESGGSVVRP | 5806 | MGWSCIILFLVATATGV |
| LALA | | GGSLRLSCAASGFTEDDYGMSWVRQAPGKGLEW | | HSSYELTQPPSVSVSPG |
| PGhL | | VSGINWNGGSTGYADSVKGRFTISRDNAKNSLY | | QTASITCSGDKLGDKYA |
| 2 | | LQMNSLRAEDTALYYCAREDYGDPGWFDPWGQG | | CWYQQKPGQSPVLVIYQ |
| | | TLVTVSSASTKGPSVFPLAPSSKSTSGGTAALG | | DSKRPSGIPERFSGSNS |
| | | CLVKDYFPEPVTVSWNSGALTSGVHTFPAVLQS | | GNTATLTISGTQAMDEA |
| | | SGLYSLSSVVTVPSSSLGTQTYICNVNHKPSNT | | DYYCQAWDSSTYVFGTG |
| | | KVDKKVEPKSCDKTHTCPPCPAPEAAGGPSVEL | | TKVTVLGQPKAAPSVTL |
| | | FPPKPKDTLMISRTPEVTCVVVDVSHEDPEVKF | | FPPSSEELQANKATLVC |
| | | NWYVDGVEVHNAKTKPREEQYNSTYRVVSVLTV | | LISDFYPGAVTVAWKAD |
| | | LHQDWLNGKEYKCKVSNKALGAPIEKTISKAKG | | SSPVKAGVETTTPSKQS |
| | | QPREPQVYTLPPSREEMTKNQVSLTCLVKGFYP | | NNKYAASSYLSLTPEQW |
| | | SDIAVEWESNGQPENNYKTTPPVLDSDGSFFLY | | KSHRSYSCQVTHEGSTV |
| | | SKLTVDKSRWQQGNVFSCSVMHEALHNHYTQKS | | EKTVAPTECS |
| | | LSLSPGK | | |
| A4- | 5619 | MGWSCIILFLVATATGVHSQLQLQESGPGLVKP | 5807 | MGWSCIILFLVATATGV |
| LALA | | SETLSLTCTVSGGSISSSSYYWGWIRQPPGKGL | | HSSYELTQPSSVSVSPG |
| PGhL | | EWIGSIYYSGSTYYNPSLKSRVTISVDTSKNQF | | QTARITCSGDVLAKKYA |
| 2 | | SLKLSSVTAADTAVYYCATLTGDGDYWGQGTLV | | RWFQQKPGQAPVLVIYK |
| | | TVSSASTKGPSVFPLAPSSKSTSGGTAALGCLV | | DSERPSGIPERFSGSSS |
| | | KDYFPEPVTVSWNSGALTSGVHTFPAVLQSSGL | | GTTVTLTISGAQVEDEA |
| | | YSLSSVVTVPSSSLGTQTYICNVNHKPSNTKVD | | DYYCYSAADNNLVFGGG |
| | | KKVEPKSCDKTHTCPPCPAPEAAGGPSVFLFPP | | TKLTVLGQPKAAPSVTL |
| | | KPKDTLMISRTPEVTCVVVDVSHEDPEVKENWY | | FPPSSEELQANKATLVC |
| | | VDGVEVHNAKTKPREEQYNSTYRVVSVLTVLHQ | | LISDFYPGAVTVAWKAD |
| | | DWLNGKEYKCKVSNKALGAPIEKTISKAKGQPR | | SSPVKAGVETTTPSKQS |
| | | EPQVYTLPPSREEMTKNQVSLTCLVKGFYPSDI | | NNKYAASSYLSLTPEQW |
| | | AVEWESNGQPENNYKTTPPVLDSDGSFFLYSKL | | KSHRSYSCQVTHEGSTV |
| | | TVDKSRWQQGNVFSCSVMHEALHNHYTQKSLSL | | EKTVAPTECS |
| | | SPGK | | |
| A5- | 5620 | MGWSCIILFLVATATGVHSEVQLVESGGGLVKP | 5808 | MGWSCIILFLVATATGV |
| LALA | | GGSLRLSCAASGFTFSSYSMNWVRQAPGKGLEW | | HSSSELTQDPAVSVALG |
| PGhL | | VSSISSSSSYIYYADSVKGRFTISRDNAKNSLY | | QTVRITCQGDSLRSYYA |
| 2 | | LQMNSLRAEDTAVYYCARDRSSSWYSFDYWGQG | | SWYQQKPGQAPVLVIYG |
| | | TLVTVSSASTKGPSVFPLAPSSKSTSGGTAALG | | KNNRPSGIPDRESGSSS |
| | | CLVKDYFPEPVTVSWNSGALTSGVHTFPAVLQS | | GNTASLTITGAQAEDEA |
| | | SGLYSLSSVVTVPSSSLGTQTYICNVNHKPSNT | | DYYCNSRDSSGNHRVFG |
| | | KVDKKVEPKSCDKTHTCPPCPAPEAAGGPSVEL | | GGTKLTVLGQPKAAPSV |
| | | FPPKPKDTLMISRTPEVTCVVVDVSHEDPEVKF | | TLFPPSSEELQANKATL |
| | | NWYVDGVEVHNAKTKPREEQYNSTYRVVSVLTV | | VCLISDFYPGAVTVAWK |
| | | LHQDWLNGKEYKCKVSNKALGAPIEKTISKAKG | | ADSSPVKAGVETTTPSK |
| | | QPREPQVYTLPPSREEMTKNQVSLTCLVKGFYP | | QSNNKYAASSYLSLTPE |
| | | SDIAVEWESNGQPENNYKTTPPVLDSDGSFFLY | | QWKSHRSYSCQVTHEGS |
| | | SKLTVDKSRWQQGNVFSCSVMHEALHNHYTQKS | | TVEKTVAPTECS |
| | | LSLSPGK | | |
| A6- | 5621 | MGWSCIILFLVATATGVHSEVQLVESGGGLVTP | 5809 | MGWSCIILFLVATATGV |
| LALA | | GGSLRLSCAASGFTENNYSMNWVRQAPGKGLEW | | HSQSALTQPRSVSGSPG |
| PGhL | | VSSISSSSSYIYYADSVKGRFTISRDNAKNSLY | | QSVTISCTGTSSDVGGY |
| 2 | | LPMISLRAEDTAVYYCARDGITGTTFYFDYWGQ | | NYVSWYQQHPGKAPKLM |
| | | GTLVTVSSASTKGPSVFPLAPSSKSTSGGTAAL | | IYDVSKRPSGVPDRESG |
| | | GCLVKDYFPEPVTVSWNSGALTSGVHTFPAVLQ | | SKSGNTASLTISGLQAE |
| | | SSGLYSLSSVVTVPSSSLGTQTYICNVNHKPSN | | DEADYYCCSYAGSYTWV |
| | | TKVDKKVEPKSCDKTHTCPPCPAPEAAGGPSVF | | FGGGTKLTVLGQPKAAP |
| | | LFPPKPKDTLMISRTPEVTCVVVDVSHEDPEVK | | SVTLFPPSSEELQANKA |
| | | FNWYVDGVEVHNAKTKPREEQYNSTYRVVSVLT | | TLVCLISDFYPGAVTVA |
| | | VLHQDWLNGKEYKCKVSNKALGAPIEKTISKAK | | WKADSSPVKAGVETTTP |
| | | GQPREPQVYTLPPSREEMTKNQVSLTCLVKGFY | | SKQSNNKYAASSYLSLT |
| | | PSDIAVEWESNGQPENNYKTTPPVLDSDGSFFL | | PEQWKSHRSYSCQVTHE |
| | | YSKLTVDKSRWQQGNVFSCSVMHEALHNHYTQK | | GSTVEKTVAPTECS |
| | | SLSLSPGK | | |
| A7- | 5622 | MGWSCIILFLVATATGVHSQVQLVESGGGVVQP | 5810 | MGWSCIILFLVATATGV |
| LALA | | GRSLRLSCAASGFTFSSYGMHWVRQAPGKGLEW | | HSQSALTQPPSASGSPG |
| PGhL | | VAVIWYDGSNKYYADSVKGRFTISRDNSKNTLY | | QSVTISCTGTSSDVGGY |
| 2 | | LQMNSLRAEDTAVYYCASIAAAGRDYWGQGTLV | | NYVSWYQQHPGKAPKLM |
| | | TVSSASTKGPSVFPLAPSSKSTSGGTAALGCLV | | IYEVSKRPSGVPDRFSG |
| | | KDYFPEPVTVSWNSGALTSGVHTFPAVLQSSGL | | SKSGNTASLTVSGLQAE |
| | | YSLSSVVTVPSSSLGTQTYICNVNHKPSNTKVD | | DEADYYCSSYAGSNNLV |
| | | KKVEPKSCDKTHTCPPCPAPEAAGGPSVFLFPP | | FGGGTKLTVLGQPKAAP |
| | | KPKDTLMISRTPEVTCVVVDVSHEDPEVKENWY | | SVTLFPPSSEELQANKA |
| | | VDGVEVHNAKTKPREEQYNSTYRVVSVLTVLHQ | | TLVCLISDFYPGAVTVA |
| | | DWLNGKEYKCKVSNKALGAPIEKTISKAKGQPR | | WKADSSPVKAGVETTTP |
| | | EPQVYTLPPSREEMTKNQVSLTCLVKGFYPSDI | | SKQSNNKYAASSYLSLT |
| | | AVEWESNGQPENNYKTTPPVLDSDGSFFLYSKL | | PEQWKSHRSYSCQVTHE |
| | | TVDKSRWQQGNVFSCSVMHEALHNHYTQKSLSL | | GSTVEKTVAPTECS |
| | | SPGK | | |
| A8- | 5623 | MGWSCIILFLVATATGVHSEVQLVESGGGLVQP | 5811 | MGWSCIILFLVATATGV |
| LALA | | GGSLRLSCAASGFTFSSYAMSWVRQAPGKGLEW | | HSQSALTQPASVSGSPG |
| PGhL | | VSAISGSGGSTYYADSVKGRFTISRDNSKNTLY | | QSITISCTGTSSDVGGY |
| 2 | | LQMNSLRAEDTAVYYCAKAPELRFDYWGQGTLV | | NYVSWYQQHPGKAPKLM |
| | | TVSSASTKGPSVFPLAPSSKSTSGGTAALGCLV | | IYEVSNRPSGVSNRFSG |
| | | KDYFPEPVTVSWNSGALTSGVHTFPAVLQSSGL | | SKSGNTASLTISGLQAE |
| | | YSLSSVVTVPSSSLGTQTYICNVNHKPSNTKVD | | DEADYYCSSYTSSSTYV |
| | | KKVEPKSCDKTHTCPPCPAPEAAGGPSVFLFPP | | FGTGTKVTVLGQPKAAP |
| | | KPKDTLMISRTPEVTCVVVDVSHEDPEVKENWY | | SVTLFPPSSEELQANKA |
| | | VDGVEVHNAKTKPREEQYNSTYRVVSVLTVLHQ | | TLVCLISDFYPGAVTVA |
| | | DWLNGKEYKCKVSNKALGAPIEKTISKAKGQPR | | WKADSSPVKAGVETTTP |
| | | EPQVYTLPPSREEMTKNQVSLTCLVKGFYPSDI | | SKQSNNKYAASSYLSLT |
| | | AVEWESNGQPENNYKTTPPVLDSDGSFFLYSKL | | PEQWKSHRSYSCQVTHE |
| | | TVDKSRWQQGNVFSCSVMHEALHNHYTQKSLSL | | GSTVEKTVAPTECS |
| | | SPGK | | |
| A9- | 5624 | MGWSCIILFLVATATGVHSQVQLVQSGAEVKKP | 5812 | MGWSCIILFLVATATGV |
| LALA | | GASVKVSCKASGYTFTSYGISWVRQAPGQGLEW | | HSQSALTQPASVSGSPG |
| PGhL | | MGWISPYNGNTNYAQNLQDRVTMITDTSTTTAY | | QSITISCTGTSSDVGGY |
| 2 | | MELRSLRSDDTAVYYCARNHYYYMDVWGKGTTV | | NYVSWYQQHPGKAPKLM |
| | | TVSSASTKGPSVFPLAPSSKSTSGGTAALGCLV | | IYDVSKRPSGVSNRFSG |
| | | KDYFPEPVTVSWNSGALTSGVHTFPAVLQSSGL | | SKSGNTASLTISGLQAE |
| | | YSLSSVVTVPSSSLGTQTYICNVNHKPSNTKVD | | DEADYYCCSYAGSSTYV |
| | | KKVEPKSCDKTHTCPPCPAPEAAGGPSVFLFPP | | VFGGGTKLTVLGQPKAA |
| | | KPKDTLMISRTPEVTCVVVDVSHEDPEVKENWY | | PSVTLFPPSSEELQANK |
| | | VDGVEVHNAKTKPREEQYNSTYRVVSVLTVLHQ | | ATLVCLISDFYPGAVTV |
| | | DWLNGKEYKCKVSNKALGAPIEKTISKAKGQPR | | AWKADSSPVKAGVETTT |
| | | EPQVYTLPPSREEMTKNQVSLTCLVKGFYPSDI | | PSKQSNNKYAASSYLSL |
| | | AVEWESNGQPENNYKTTPPVLDSDGSFFLYSKL | | TPEQWKSHRSYSCQVTH |
| | | TVDKSRWQQGNVFSCSVMHEALHNHYTQKSLSL | | EGSTVEKTVAPTECS |
| | | SPGK | | |
| A10- | 5625 | MGWSCIILFLVATATGVHSQVQLQQWGAGLLKP | 5813 | MGWSCIILFLVATATGV |
| LALA | | SETLSLTCAVYGGSFSGYYWSWIRQPPGKGLEW | | HSSSELTQDPAVSVALG |
| PGhL | | IGEINHSGSTNYNPSLKSRVTISVDTSKNQFSL | | QTVRITCQGDSLRSYYA |
| 2 | | KLSSVTAADTAVYYCARGELGIGYWYFDLWGRG | | SWYQQKPGQAPVLVIYG |
| | | TLVTVSSASTKGPSVFPLAPSSKSTSGGTAALG | | KNNRPSGIPDRFSGSSS |
| | | CLVKDYFPEPVTVSWNSGALTSGVHTFPAVLQS | | GNTASLTITGAQAEDEA |
| | | SGLYSLSSVVTVPSSSLGTQTYICNVNHKPSNT | | DYYCNSRDSSGNHVVFG |
| | | KVDKKVEPKSCDKTHTCPPCPAPEAAGGPSVFL | | GGTKLTVLGQPKAAPSV |
| | | FPPKPKDTLMISRTPEVTCVVVDVSHEDPEVKF | | TLFPPSSEELQANKATL |
| | | NWYVDGVEVHNAKTKPREEQYNSTYRVVSVLTV | | VCLISDFYPGAVTVAWK |
| | | LHQDWLNGKEYKCKVSNKALGAPIEKTISKAKG | | ADSSPVKAGVETTTPSK |
| | | QPREPQVYTLPPSREEMTKNQVSLTCLVKGFYP | | QSNNKYAASSYLSLTPE |
| | | SDIAVEWESNGQPENNYKTTPPVLDSDGSFFLY | | QWKSHRSYSCQVTHEGS |
| | | SKLTVDKSRWQQGNVFSCSVMHEALHNHYTQKS | | TVEKTVAPTECS |
| | | LSLSPGK | | |
| A11- | 5626 | MGWSCIILFLVATATGVHSQVQLVESGGGVVQP | 5814 | MGWSCIILFLVATATGV |
| LALA | | GRSLRLSCAASGFTFSSYGMHWVRQAPGKGLEW | | HSSYVLTQPPSVSVAPG |
| PGhL | | VAVIWYDGSNKYYADSVKGRFTISRDNSKNTLY | | KTARITCGGNNIGSKSV |
| 2 | | LQMNSLRAEDTAVYYCARDTGITMVRGVFDYWG | | HWYQQKPGQAPVLVIYY |
| | | QGTLVTVSSASTKGPSVFPLAPSSKSTSGGTAA | | DSDRPSGIPERFSGSNS |
| | | LGCLVKDYFPEPVTVSWNSGALTSGVHTFPAVL | | GNTATLTISRVEAGDEA |
| | | QSSGLYSLSSVVTVPSSSLGTQTYICNVNHKPS | | DYYCQVWDSSSDHPVFG |
| | | NTKVDKKVEPKSCDKTHTCPPCPAPEAAGGPSV | | GGTKLTVLGQPKAAPSV |
| | | FLFPPKPKDTLMISRTPEVTCVVVDVSHEDPEV | | TLFPPSSEELQANKATL |
| | | KFNWYVDGVEVHNAKTKPREEQYNSTYRVVSVL | | VCLISDFYPGAVTVAWK |
| | | TVLHQDWLNGKEYKCKVSNKALGAPIEKTISKA | | ADSSPVKAGVETTTPSK |
| | | KGQPREPQVYTLPPSREEMTKNQVSLTCLVKGF | | QSNNKYAASSYLSLTPE |
| | | YPSDIAVEWESNGQPENNYKTTPPVLDSDGSFF | | QWKSHRSYSCQVTHEGS |
| | | LYSKLTVDKSRWQQGNVFSCSVMHEALHNHYTQ | | TVEKTVAPTECS |
| | | KSLSLSPGK | | |
| A12- | 5627 | MGWSCIILFLVATATGVHSEVQLVESGGDLVQP | 5815 | MGWSCIILFLVATATGV |
| LALA | | GGSLKLSCAASGFSFSGSTLHWVRQASGKGLEW | | HSQAVLTQPASLSASPG |
| PGhL | | IGHIRSKPNNYATLYGASVKGRFTISRDDSKNT | | ASASLTCTLRSGINVGT |
| 2 | | AYLQMNSLKIEDTAVYYCNGVYGGSSYFFDYWG | | YRIYWYQQKPGSPPQYL |
| | | QGTLVTVSSASTKGPSVFPLAPSSKSTSGGTAA | | LRYKSDSDKQQGSGVPS |
| | | LGCLVKDYFPEPVTVSWNSGALTSGVHTFPAVL | | RFSGSKDASANAGILLI |
| | | QSSGLYSLSSVVTVPSSSLGTQTYICNVNHKPS | | SGLQSEDEADYYCMIWH |
| | | NTKVDKKVEPKSCDKTHTCPPCPAPEAAGGPSV | | SSAVVFGGGTKLTVLGQ |
| | | FLFPPKPKDTLMISRTPEVTCVVVDVSHEDPEV | | PKAAPSVTLFPPSSEEL |
| | | KFNWYVDGVEVHNAKTKPREEQYNSTYRVVSVL | | QANKATLVCLISDFYPG |
| | | TVLHQDWLNGKEYKCKVSNKALGAPIEKTISKA | | AVTVAWKADSSPVKAGV |
| | | KGQPREPQVYTLPPSREEMTKNQVSLTCLVKGF | | ETTTPSKQSNNKYAASS |
| | | YPSDIAVEWESNGQPENNYKTTPPVLDSDGSFF | | YLSLTPEQWKSHRSYSC |
| | | LYSKLTVDKSRWQQGNVFSCSVMHEALHNHYTQ | | QVTHEGSTVEKTVAPTE |
| | | KSLSLSPGK | | CS |
| A13- | 5628 | MGWSCIILFLVATATGVHSQVQLVQSGAEVKKP | 5816 | MGWSCIILFLVATATGV |
| LALA | | GASVKVSCKASGYTFTGYYMHWVRQAPGQGLEW | | HSSSELTQDPAVSVALG |
| PGhL | | MGWINPNSGGTNYAQKFQGRVTMTRDTSISTAY | | QTVRITCQGDSLRSYYA |
| 2 | | MELSRLRSDDTAVYYCARDETTIFDYWGQGTLV | | SWYQQKPGQAPVLVIYG |
| | | TVSSASTKGPSVFPLAPSSKSTSGGTAALGCLV | | KNNRPSGIPDRFSGSSS |
| | | KDYFPEPVTVSWNSGALTSGVHTFPAVLQSSGL | | GNTASLTITGAQAEDEA |
| | | YSLSSVVTVPSSSLGTQTYICNVNHKPSNTKVD | | DYYCNSRDSSGNWVFGG |
| | | KKVEPKSCDKTHTCPPCPAPEAAGGPSVFLFPP | | GTKLTVLGQPKAAPSVT |
| | | KPKDTLMISRTPEVTCVVVDVSHEDPEVKENWY | | LFPPSSEELQANKATLV |
| | | VDGVEVHNAKTKPREEQYNSTYRVVSVLTVLHQ | | CLISDFYPGAVTVAWKA |
| | | DWLNGKEYKCKVSNKALGAPIEKTISKAKGQPR | | DSSPVKAGVETTTPSKQ |
| | | EPQVYTLPPSREEMTKNQVSLTCLVKGFYPSDI | | SNNKYAASSYLSLTPEQ |
| | | AVEWESNGQPENNYKTTPPVLDSDGSFFLYSKL | | WKSHRSYSCQVTHEGST |
| | | TVDKSRWQQGNVFSCSVMHEALHNHYTQKSLSL | | VEKTVAPTECS |
| | | SPGK | | |
| A14- | 5629 | MGWSCIILFLVATATGVHSQVQLVQSGAEVTKP | 5817 | MGWSCIILFLVATATGV |
| LALA | | GASVKVSCKASGYTFINYGISWVRQAPGQGLEW | | HSSYELTQPPSVSVSPG |
| PGhL | | MGWISAYSGNRNYAQKFQDRVIMTTDTFTNTAY | | QTARITCSGDALPKKYA |
| 2 | | MELRSLRSDDTAVYYCARLGCNGTSCYTSWYYH | | YWYQQKSGQAPVLVIYE |
| | | FYMDVWGKGTTVTVSSASTKGPSVFPLAPSSKS | | DSKRPSGIPERFSGSSS |
| | | TSGGTAALGCLVKDYFPEPVTVSWNSGALTSGV | | GTMATLTISGAQVEDEA |
| | | HTFPAVLQSSGLYSLSSVVTVPSSSLGTQTYIC | | DYYCYSTDSSGNHSWVF |
| | | NVNHKPSNTKVDKKVEPKSCDKTHTCPPCPAPE | | GGGTKLTVLGQPKAAPS |
| | | AAGGPSVFLFPPKPKDTLMISRTPEVTCVVVDV | | VTLFPPSSEELQANKAT |
| | | SHEDPEVKFNWYVDGVEVHNAKTKPREEQYNST | | LVCLISDFYPGAVTVAW |
| | | YRVVSVLTVLHQDWLNGKEYKCKVSNKALGAPI | | KADSSPVKAGVETTTPS |
| | | EKTISKAKGQPREPQVYTLPPSREEMTKNQVSL | | KQSNNKYAASSYLSLTP |
| | | TCLVKGFYPSDIAVEWESNGQPENNYKTTPPVL | | EQWKSHRSYSCQVTHEG |
| | | DSDGSFFLYSKLTVDKSRWQQGNVFSCSVMHEA | | STVEKTVAPTECS |
| | | LHNHYTQKSLSLSPGK | | |
| A15- | 5630 | MGWSCIILFLVATATGVHSQVQLVESGGGVVQP | 5818 | MGWSCIILFLVATATGV |
| LALA | | GRSLRLSCAASGFTFSSYGMHWVRQAPGKGLEW | | HSQSALTQPASVSGSPG |
| PGhL | | VAVIWYDGSNKYYADSVKGRFTISRDNSKNTLY | | QSITISCTGTSSDVGGY |
| 2 | | LQMNSLRAEDTAVYYCARDEDYYGSGSYSFDYW | | NYVSWYQQHPGKAPKLM |
| | | GQGTLVTVSSASTKGPSVFPLAPSSKSTSGGTA | | IYEVSNRPSGVSNRFSG |
| | | ALGCLVKDYFPEPVTVSWNSGALTSGVHTFPAV | | SKSGNTASLTISGLQAE |
| | | LQSSGLYSLSSVVTVPSSSLGTQTYICNVNHKP | | DEADYYCSSYTSSSTLV |
| | | SNTKVDKKVEPKSCDKTHTCPPCPAPEAAGGPS | | FGGGTKLTVLGQPKAAP |
| | | VFLFPPKPKDTLMISRTPEVTCVVVDVSHEDPE | | SVTLFPPSSEELQANKA |
| | | VKFNWYVDGVEVHNAKTKPREEQYNSTYRVVSV | | TLVCLISDFYPGAVTVA |
| | | LTVLHQDWLNGKEYKCKVSNKALGAPIEKTISK | | WKADSSPVKAGVETTTP |
| | | AKGQPREPQVYTLPPSREEMTKNQVSLTCLVKG | | SKQSNNKYAASSYLSLT |
| | | FYPSDIAVEWESNGQPENNYKTTPPVLDSDGSF | | PEQWKSHRSYSCQVTHE |
| | | FLYSKLTVDKSRWQQGNVFSCSVMHEALHNHYT | | GSTVEKTVAPTECS |
| | | QKSLSLSPGK | | |
| A16- | 5631 | MGWSCIILFLVATATGVHSQVQLQESGPGLVKP | 5819 | MGWSCIILFLVATATGV |
| LALA | | SGTLSLTCAVSGGSISSSNWWSWVRQPPGKGLE | | HSQAVLTQPASLSASPG |
| PGhL | | WIGEIYHSGSTNYNPSLKSRVTISVDKSKNQFS | | ASASLTCTLRSGINVGT |
| 2 | | LKLSSVTAADTAVYYCARRGAARPFDYWGQGTL | | YRIYWYQQKPGSPPQYL |
| | | VTVSSASTKGPSVFPLAPSSKSTSGGTAALGCL | | LRYKSDSDKQQGSGVPS |
| | | VKDYFPEPVTVSWNSGALTSGVHTFPAVLQSSG | | RFSGSKDASANAGILLI |
| | | LYSLSSVVTVPSSSLGTQTYICNVNHKPSNTKV | | SGLQSEDEADYYCMIWH |
| | | DKKVEPKSCDKTHTCPPCPAPEAAGGPSVFLFP | | SSAYVVFGGGTKLTVLG |
| | | PKPKDTLMISRTPEVTCVVVDVSHEDPEVKENW | | QPKAAPSVTLFPPSSEE |
| | | YVDGVEVHNAKTKPREEQYNSTYRVVSVLTVLH | | LQANKATLVCLISDFYP |
| | | QDWLNGKEYKCKVSNKALGAPIEKTISKAKGQP | | GAVTVAWKADSSPVKAG |
| | | REPQVYTLPPSREEMTKNQVSLTCLVKGFYPSD | | VETTTPSKQSNNKYAAS |
| | | IAVEWESNGQPENNYKTTPPVLDSDGSFFLYSK | | SYLSLTPEQWKSHRSYS |
| | | LTVDKSRWQQGNVFSCSVMHEALHNHYTQKSLS | | CQVTHEGSTVEKTVAPT |
| | | LSPGK | | ECS |
| A17- | 5632 | MGWSCIILFLVATATGVHSQITLKESGPTLVKP | 5820 | MGWSCIILFLVATATGV |
| LALA | | TQTLTLTCTFSGFSLSTSGVGVGWIRQPPGKAL | | HSSYELTQPPSVSVSPG |
| PGhL | | EWLALIYWNDDKRYSPSLKSRLTITKDTSKNQV | | QTASITCSGDKLGDKYA |
| 2 | | VLTMTNMDPVDTATYYCAHSNWNYGYFDLWGRG | | CWYQQKPGQSPVLVIYQ |
| | | TLVTVSSASTKGPSVFPLAPSSKSTSGGTAALG | | DSKRPSGIPERFSGSNS |
| | | CLVKDYFPEPVTVSWNSGALTSGVHTFPAVLQS | | GNTATLTISGTQAMDEA |
| | | SGLYSLSSVVTVPSSSLGTQTYICNVNHKPSNT | | DYYCQAWDSSTAWVEGG |
| | | KVDKKVEPKSCDKTHTCPPCPAPEAAGGPSVEL | | GTKLTVLGQPKAAPSVT |
| | | FPPKPKDTLMISRTPEVTCVVVDVSHEDPEVKF | | LFPPSSEELQANKATLV |
| | | NWYVDGVEVHNAKTKPREEQYNSTYRVVSVLTV | | CLISDFYPGAVTVAWKA |
| | | LHQDWLNGKEYKCKVSNKALGAPIEKTISKAKG | | DSSPVKAGVETTTPSKQ |
| | | QPREPQVYTLPPSREEMTKNQVSLTCLVKGFYP | | SNNKYAASSYLSLTPEQ |
| | | SDIAVEWESNGQPENNYKTTPPVLDSDGSFFLY | | WKSHRSYSCQVTHEGST |
| | | SKLTVDKSRWQQGNVFSCSVMHEALHNHYTQKS | | VEKTVAPTECS |
| | | LSLSPGK | | |
| A18- | 5633 | MGWSCIILFLVATATGVHSEVQLVESGGGLVQP | 5821 | MGWSCIILFLVATATGV |
| LALA | | GGSLRLSCAASGFTFSSYAMSWVRQAPGKGLEW | | HSSYELTQPPSVSVSPG |
| PGhL | | VSAISGSGGSTYYADSVKGRFTISRDNSKNTLY | | QTARITCSGDALPKKYA |
| 2 | | LQMNSLRAEDTAVYYCAKKDIVATHFDYWGQGT | | YWYQQKSGQAPVLVIYE |
| | | LVTVSSASTKGPSVFPLAPSSKSTSGGTAALGC | | DSKRPSGIPERFSGSSS |
| | | LVKDYFPEPVTVSWNSGALTSGVHTFPAVLQSS | | GTMATLTISGAQVEDEA |
| | | GLYSLSSVVTVPSSSLGTQTYICNVNHKPSNTK | | DYYCYSTDSSGNHKVFG |
| | | VDKKVEPKSCDKTHTCPPCPAPEAAGGPSVFLF | | GGTKLTVLGQPKAAPSV |
| | | PPKPKDTLMISRTPEVTCVVVDVSHEDPEVKEN | | TLFPPSSEELQANKATL |
| | | WYVDGVEVHNAKTKPREEQYNSTYRVVSVLTVL | | VCLISDFYPGAVTVAWK |
| | | HQDWLNGKEYKCKVSNKALGAPIEKTISKAKGQ | | ADSSPVKAGVETTTPSK |
| | | PREPQVYTLPPSREEMTKNQVSLTCLVKGFYPS | | QSNNKYAASSYLSLTPE |
| | | DIAVEWESNGQPENNYKTTPPVLDSDGSFFLYS | | QWKSHRSYSCQVTHEGS |
| | | KLTVDKSRWQQGNVFSCSVMHEALHNHYTQKSL | | TVEKTVAPTECS |
| | | SLSPGK | | |
| A19- | 5634 | MGWSCIILFLVATATGVHSEVQLVESGGGLVKP | 5822 | MGWSCIILFLVATATGV |
| LALA | | GGSLRLSCAASGFTFSNAWMSWVRQAPGKGLEW | | HSSSELTQDPAVSVALG |
| PGhL | | VGRIKSKTDGGTTDYAAPVKGRFTISRDDSKNT | | QTVRITCQGDSLRSYYA |
| 2 | | LYLQMNSLKTEDTAVYYCTTADYDFWSGYYMDV | | SWYQQKPGQAPVLVIYG |
| | | WGKGTTVTVSSASTKGPSVFPLAPSSKSTSGGT | | KNNRPSGIPDRFSGSSS |
| | | AALGCLVKDYFPEPVTVSWNSGALTSGVHTFPA | | GNTASLTITGAQAEDEA |
| | | VLQSSGLYSLSSVVTVPSSSLGTQTYICNVNHK | | DYYCNSRDSSGNHWVFG |
| | | PSNTKVDKKVEPKSCDKTHTCPPCPAPEAAGGP | | GGTKLTVLGQPKAAPSV |
| | | SVFLFPPKPKDTLMISRTPEVTCVVVDVSHEDP | | TLFPPSSEELQANKATL |
| | | EVKFNWYVDGVEVHNAKTKPREEQYNSTYRVVS | | VCLISDFYPGAVTVAWK |
| | | VLTVLHQDWLNGKEYKCKVSNKALGAPIEKTIS | | ADSSPVKAGVETTTPSK |
| | | KAKGQPREPQVYTLPPSREEMTKNQVSLTCLVK | | QSNNKYAASSYLSLTPE |
| | | GFYPSDIAVEWESNGQPENNYKTTPPVLDSDGS | | QWKSHRSYSCQVTHEGS |
| | | FFLYSKLTVDKSRWQQGNVFSCSVMHEALHNHY | | TVEKTVAPTECS |
| | | TQKSLSLSPGK | | |
| A20- | 5635 | MGWSCIILFLVATATGVHSEVQLVESGGGLVQP | 5823 | MGWSCIILFLVATATGV |
| LALA | | GGSLRLSCAASGFTFSSYSMNWVRQAPGKGLEW | | HSQSALTQPRSVSGSPG |
| PGhL | | VSYISSSSSTIYYADSVKGRFTISRDNAKNSLY | | QSVTISCTGTSSDVGGY |
| 2 | | LQMNSLRDEDTAVYYCARDRYNFDYWGQGTLVT | | NYVSWYQQHPGKAPKLM |
| | | VSSASTKGPSVFPLAPSSKSTSGGTAALGCLVK | | IYDVSKRPSGVPDRFSG |
| | | DYFPEPVTVSWNSGALTSGVHTFPAVLQSSGLY | | SKSGNTASLTISGLQAE |
| | | SLSSVVTVPSSSLGTQTYICNVNHKPSNTKVDK | | DEADYYCCSYAGSSWVF |
| | | KVEPKSCDKTHTCPPCPAPEAAGGPSVFLFPPK | | GGGTKLTVLGQPKAAPS |
| | | PKDTLMISRTPEVTCVVVDVSHEDPEVKENWYV | | VTLFPPSSEELQANKAT |
| | | DGVEVHNAKTKPREEQYNSTYRVVSVLTVLHQD | | LVCLISDFYPGAVTVAW |
| | | WLNGKEYKCKVSNKALGAPIEKTISKAKGQPRE | | KADSSPVKAGVETTTPS |
| | | PQVYTLPPSREEMTKNQVSLTCLVKGFYPSDIA | | KQSNNKYAASSYLSLTP |
| | | VEWESNGQPENNYKTTPPVLDSDGSFFLYSKLT | | EQWKSHRSYSCQVTHEG |
| | | VDKSRWQQGNVFSCSVMHEALHNHYTQKSLSLS | | STVEKTVAPTECS |
| | | PGK | | |
| A21- | 5636 | MGWSCIILFLVATATGVHSEVQLVESGGGVVRP | 5824 | MGWSCIILFLVATATGV |
| LALA | | GGSLRLSCAASGFTFDDYGMSWVRQAPGKGLEW | | HSQSALTQPASVSGSPG |
| PGhL | | VSGINWNGGSTGYADSVKGRFTISRDNAKNSLY | | QSITISCTGTSSDVGGY |
| 2 | | LQMNSLRAEDTALYYCARGGDTAMVTVFDYWGQ | | NYVSWYQQHPGKAPKLM |
| | | GTLVTVSSASTKGPSVFPLAPSSKSTSGGTAAL | | IYEVSNRPSGVSNRFSG |
| | | GCLVKDYFPEPVTVSWNSGALTSGVHTFPAVLQ | | SKSGNTASLTISGLQAE |
| | | SSGLYSLSSVVTVPSSSLGTQTYICNVNHKPSN | | DEADYYCSSYTSSSTLV |
| | | TKVDKKVEPKSCDKTHTCPPCPAPEAAGGPSVF | | FGTGTKVTVLGQPKAAP |
| | | LFPPKPKDTLMISRTPEVTCVVVDVSHEDPEVK | | SVTLFPPSSEELQANKA |
| | | FNWYVDGVEVHNAKTKPREEQYNSTYRVVSVLT | | TLVCLISDFYPGAVTVA |
| | | VLHQDWLNGKEYKCKVSNKALGAPIEKTISKAK | | WKADSSPVKAGVETTTP |
| | | GQPREPQVYTLPPSREEMTKNQVSLTCLVKGFY | | SKQSNNKYAASSYLSLT |
| | | PSDIAVEWESNGQPENNYKTTPPVLDSDGSFFL | | PEQWKSHRSYSCQVTHE |
| | | YSKLTVDKSRWQQGNVFSCSVMHEALHNHYTQK | | GSTVEKTVAPTECS |
| | | SLSLSPGK | | |
| A22- | 5637 | MGWSCIILFLVATATGVHSEVQLVQSGAEVKKP | 5825 | MGWSCIILFLVATATGV |
| LALA | | GESLKISCKGSGYSFTSYWIGWVRQMSGKGLEW | | HSQSALTQPASVSGSPG |
| PGhL | | MGIIYPSDSDTRYSPSFQGQVTISADKSISTAY | | QSITISCTGTSSDVGGY |
| 2 | | LQWSSLKASDTAMYYCARQINWGAIDYWGQGTL | | NYVSWYQQHPGKAPKLM |
| | | VTVSSASTKGPSVFPLAPSSKSTSGGTAALGCL | | IYDVSKRPSGVSNRFSG |
| | | VKDYFPEPVTVSWNSGALTSGVHTFPAVLQSSG | | SKSGNTASLTISGLQAE |
| | | LYSLSSVVTVPSSSLGTQTYICNVNHKPSNTKV | | DEADYYCCSYAGSSTFV |
| | | DKKVEPKSCDKTHTCPPCPAPEAAGGPSVFLFP | | VFGGGTKLTVLGQPKAA |
| | | PKPKDTLMISRTPEVTCVVVDVSHEDPEVKENW | | PSVTLFPPSSEELQANK |
| | | YVDGVEVHNAKTKPREEQYNSTYRVVSVLTVLH | | ATLVCLISDFYPGAVTV |
| | | QDWLNGKEYKCKVSNKALGAPIEKTISKAKGQP | | AWKADSSPVKAGVETTT |
| | | REPQVYTLPPSREEMTKNQVSLTCLVKGFYPSD | | PSKQSNNKYAASSYLSL |
| | | IAVEWESNGQPENNYKTTPPVLDSDGSFFLYSK | | TPEQWKSHRSYSCQVTH |
| | | LTVDKSRWQQGNVFSCSVMHEALHNHYTQKSLS | | EGSTVEKTVAPTECS |
| | | LSPGK | | |
| A23- | 5638 | MGWSCIILFLVATATGVHSQVQLVQSGAEVKKP | 5826 | MGWSCIILFLVATATGV |
| LALA | | GASVKVSCKASGYTFTSYGISWVRQAPGQGLEW | | HSQSALTQPASVSGSPG |
| PGhL | | MGWISVYNGNTNYAQKLQGRVTMTTDTSTSTAY | | QSITISCTGTSSDVGGY |
| 2 | | MELRSLRSDDTAVYYCARQITATRGFDYWGQGT | | NYVSWYQQHPGKAPKLM |
| | | LVTVSSASTKGPSVFPLAPSSKSTSGGTAALGC | | IYDVSKRPSGVSNRFSG |
| | | LVKDYFPEPVTVSWNSGALTSGVHTFPAVLQSS | | SKSGNTASLTISGLQAE |
| | | GLYSLSSVVTVPSSSLGTQTYICNVNHKPSNTK | | DEADYYCCSYAGSSTFV |
| | | VDKKVEPKSCDKTHTCPPCPAPEAAGGPSVFLF | | VFGGGTKLTVLGQPKAA |
| | | PPKPKDTLMISRTPEVTCVVVDVSHEDPEVKEN | | PSVTLFPPSSEELQANK |
| | | WYVDGVEVHNAKTKPREEQYNSTYRVVSVLTVL | | ATLVCLISDFYPGAVTV |
| | | HQDWLNGKEYKCKVSNKALGAPIEKTISKAKGQ | | AWKADSSPVKAGVETTT |
| | | PREPQVYTLPPSREEMTKNQVSLTCLVKGFYPS | | PSKQSNNKYAASSYLSL |
| | | DIAVEWESNGQPENNYKTTPPVLDSDGSFFLYS | | TPEQWKSHRSYSCQVTH |
| | | KLTVDKSRWQQGNVFSCSVMHEALHNHYTQKSL | | EGSTVEKTVAPTECS |
| | | SLSPGK | | |
| A24- | 5639 | MGWSCIILFLVATATGVHSQVQLQQSGPGLVKP | 5827 | MGWSCIILFLVATATGV |
| LALA | | SQTLSLTCAISGDSVSSNSAAWSWIRQSPSRGL | | HSQSALTQPASVSGSPG |
| PGhL | | EWLGRTYYRSKWYNDYAVSVKSRITINPDTSKN | | QSITISCTGTSSDVGGY |
| 2 | | QFSLQLNSVTPEDTAVYYCARKWELRDTFDIWG | | NYVSWYQQHPGKAPKLM |
| | | QGTMVTVSSASTKGPSVFPLAPSSKSTSGGTAA | | IYDVSKRPSGVSNRFSG |
| | | LGCLVKDYFPEPVTVSWNSGALTSGVHTFPAVL | | SKSGNTASLTISGLQAE |
| | | QSSGLYSLSSVVTVPSSSLGTQTYICNVNHKPS | | DEADYYCCSYAGSSTLG |
| | | NTKVDKKVEPKSCDKTHTCPPCPAPEAAGGPSV | | IDWVFGGGTKLTVLGQP |
| | | FLFPPKPKDTLMISRTPEVTCVVVDVSHEDPEV | | KAAPSVTLFPPSSEELQ |
| | | KFNWYVDGVEVHNAKTKPREEQYNSTYRVVSVL | | ANKATLVCLISDFYPGA |
| | | TVLHQDWLNGKEYKCKVSNKALGAPIEKTISKA | | VTVAWKADSSPVKAGVE |
| | | KGQPREPQVYTLPPSREEMTKNQVSLTCLVKGF | | TTTPSKQSNNKYAASSY |
| | | YPSDIAVEWESNGQPENNYKTTPPVLDSDGSFF | | LSLTPEQWKSHRSYSCQ |
| | | LYSKLTVDKSRWQQGNVFSCSVMHEALHNHYTQ | | VTHEGSTVEKTVAPTEC |
| | | KSLSLSPGK | | S |
| A25- | 5640 | MGWSCIILFLVATATGVHSQVQLQQWGAGLLKP | 5828 | MGWSCIILFLVATATGV |
| LALA | | SETLSLTCAVYGGSFSGYYWSWIRQPPGKGLEW | | HSQAVLTQPPSASALLG |
| PGhL | | IGEINHSGSTNYNPSLKSRVTISVDTSKNQFSL | | ASIKLTCTLSSEHSTYT |
| 2 | | KLSSVTAADTAVYYCASYGDFFDYWGQGTLVTV | | IEWYQQRPGRSPQYIMK |
| | | SSASTKGPSVFPLAPSSKSTSGGTAALGCLVKD | | VKSDGSHSKGDGIPDRF |
| | | YFPEPVTVSWNSGALTSGVHTFPAVLQSSGLYS | | MGSSSGADRYLTFSNLQ |
| | | LSSVVTVPSSSLGTQTYICNVNHKPSNTKVDKK | | SDDEAEYHCGESHTIDG |
| | | VEPKSCDKTHTCPPCPAPEAAGGPSVFLFPPKP | | QVGVVFGGGTKLTVLGQ |
| | | KDTLMISRTPEVTCVVVDVSHEDPEVKFNWYVD | | PKAAPSVTLFPPSSEEL |
| | | GVEVHNAKTKPREEQYNSTYRVVSVLTVLHQDW | | QANKATLVCLISDFYPG |
| | | LNGKEYKCKVSNKALGAPIEKTISKAKGQPREP | | AVTVAWKADSSPVKAGV |
| | | QVYTLPPSREEMTKNQVSLTCLVKGFYPSDIAV | | ETTTPSKQSNNKYAASS |
| | | EWESNGQPENNYKTTPPVLDSDGSFFLYSKLTV | | YLSLTPEQWKSHRSYSC |
| | | DKSRWQQGNVFSCSVMHEALHNHYTQKSLSLSP | | QVTHEGSTVEKTVAPTE |
| | | GK | | CS |
| A26- | 5641 | MGWSCIILFLVATATGVHSEVQLVESGGGLVKP | 5829 | MGWSCIILFLVATATGV |
| LALA | | GGSLRLSCAASGFTFSSYSMNWVRQAPGKGLEW | | HSQAVLTQPPSASALLG |
| PGhL | | VSSISSSSSYIYYADSVKGRFTISRDNAKNSLY | | ASIKLTCTLSSEHSTYT |
| 2 | | LQMNSLRAEDTAVYYCARETELTVMDVWGKGTT | | IEWYQQRPGRSPQYIMK |
| | | VTVSSASTKGPSVFPLAPSSKSTSGGTAALGCL | | VKSDGSHSKGDGIPDRF |
| | | VKDYFPEPVTVSWNSGALTSGVHTFPAVLQSSG | | MGSSSGADRYLTFSNLQ |
| | | LYSLSSVVTVPSSSLGTQTYICNVNHKPSNTKV | | SDDEAEYHCGESHTIDG |
| | | DKKVEPKSCDKTHTCPPCPAPEAAGGPSVFLFP | | QVGWVFGGGTKLTVLGQ |
| | | PKPKDTLMISRTPEVTCVVVDVSHEDPEVKENW | | PKAAPSVTLFPPSSEEL |
| | | YVDGVEVHNAKTKPREEQYNSTYRVVSVLTVLH | | QANKATLVCLISDFYPG |
| | | QDWLNGKEYKCKVSNKALGAPIEKTISKAKGQP | | AVTVAWKADSSPVKAGV |
| | | REPQVYTLPPSREEMTKNQVSLTCLVKGFYPSD | | ETTTPSKQSNNKYAASS |
| | | IAVEWESNGQPENNYKTTPPVLDSDGSFFLYSK | | YLSLTPEQWKSHRSYSC |
| | | LTVDKSRWQQGNVFSCSVMHEALHNHYTQKSLS | | QVTHEGSTVEKTVAPTE |
| | | LSPGK | | CS |
| A27- | 5642 | MGWSCIILFLVATATGVHSEVQLVESGGGLVKP | 5830 | MGWSCIILFLVATATGV |
| LALA | | GGSLRLSCAASGFTFSNAWMSWVRQAPGKGLEW | | HSSSELTQDPAVSVALG |
| PGhL | | VGRIKSKTDGGTTDYAAPVKGRFTISRDDSKNT | | QTVRITCQGDSLRSYYA |
| 2 | | LYLQMNSLKTEDTAVYYCTTDWEYYDFWSGYYS | | SWYQQKPGQAPVLVIYG |
| | | PYFDYWGQGTLVTVSSASTKGPSVFPLAPSSKS | | KNNRPSGIPDRFSGSSS |
| | | TSGGTAALGCLVKDYFPEPVTVSWNSGALTSGV | | GNTASLTITGAQAEDEA |
| | | HTFPAVLQSSGLYSLSSVVTVPSSSLGTQTYIC | | DYYCNSRDSSGNHVVFG |
| | | NVNHKPSNTKVDKKVEPKSCDKTHTCPPCPAPE | | GGTKLTVLGQPKAAPSV |
| | | AAGGPSVFLFPPKPKDTLMISRTPEVTCVVVDV | | TLFPPSSEELQANKATL |
| | | SHEDPEVKFNWYVDGVEVHNAKTKPREEQYNST | | VCLISDFYPGAVTVAWK |
| | | YRVVSVLTVLHQDWLNGKEYKCKVSNKALGAPI | | ADSSPVKAGVETTTPSK |
| | | EKTISKAKGQPREPQVYTLPPSREEMTKNQVSL | | QSNNKYAASSYLSLTPE |
| | | TCLVKGFYPSDIAVEWESNGQPENNYKTTPPVL | | QWKSHRSYSCQVTHEGS |
| | | DSDGSFFLYSKLTVDKSRWQQGNVFSCSVMHEA | | TVEKTVAPTECS |
| | | LHNHYTQKSLSLSPGK | | |
| A28- | 5643 | MGWSCIILFLVATATGVHSQVQLQESGPGLVKP | 5831 | MGWSCIILFLVATATGV |
| LALA | | SQTLSLTCTVSGGSISSGGYYWSWIRQHPGKGL | | HSSYVLTQPPSVSVPPG |
| PGhL | | EWIGYIYYIGITYYNPSLKSRVTISVDTSKNQF | | KTARITCGGNNVGSKSV |
| 2 | | SLKLSSVTAADTAVYYCARAFDYWGQGTLVTVS | | HWYQQKPGQAPVLVIYY |
| | | SASTKGPSVFPLAPSSKSTSGGTAALGCLVKDY | | DTDRPSGIPERFSGSNS |
| | | FPEPVTVSWNSGALTSGVHTFPAVLQSSGLYSL | | GNTATLSISRVEAGDEA |
| | | SSVVTVPSSSLGTQTYICNVNHKPSNTKVDKKV | | DYYCQVWDSRSDHVVFG |
| | | EPKSCDKTHTCPPCPAPEAAGGPSVFLFPPKPK | | GGTKLTVLGQPKAAPSV |
| | | DTLMISRTPEVTCVVVDVSHEDPEVKENWYVDG | | TLFPPSSEELQANKATL |
| | | VEVHNAKTKPREEQYNSTYRVVSVLTVLHQDWL | | VCLISDFYPGAVTVAWK |
| | | NGKEYKCKVSNKALGAPIEKTISKAKGQPREPQ | | ADSSPVKAGVETTTPSK |
| | | VYTLPPSREEMTKNQVSLTCLVKGFYPSDIAVE | | QSNNKYAASSYLSLTPE |
| | | WESNGQPENNYKTTPPVLDSDGSFFLYSKLTVD | | QWKSHRSYSCQVTHEGS |
| | | KSRWQQGNVFSCSVMHEALHNHYTQKSLSLSPG | | TVEKTVAPTECS |
| | | K | | |
| A29- | 5644 | MGWSCIILFLVATATGVHSQVQLQESGPGLVKP | 5832 | MGWSCIILFLVATATGV |
| LALA | | SQTLSLTCTVSGGSISSGGYYWSWIRQHPGKGL | | HSSYVLTQPPSVSVAPG |
| PGhL | | EWIGYFYYSGSTYYNPSLKSRVSISVDTSKNQF | | KTARITCGGNTFGSKTV |
| 2 | | SLRLSSVTVADTAVYYCVRAFDYWGQGTLVTVS | | HWYQQKPGQAPVLVIYY |
| | | SASTKGPSVFPLAPSSKSTSGGTAALGCLVKDY | | DSDRPSGIPERFSGSNS |
| | | FPEPVTVSWNSGALTSGVHTFPAVLQSSGLYSL | | GNTATLTISRVEAGDEA |
| | | SSVVTVPSSSLGTQTYICNVNHKPSNTKVDKKV | | DYYCQVWDLYSAHVVFG |
| | | EPKSCDKTHTCPPCPAPEAAGGPSVFLFPPKPK | | GGTQLTVLGQPKAAPSV |
| | | DTLMISRTPEVTCVVVDVSHEDPEVKENWYVDG | | TLFPPSSEELQANKATL |
| | | VEVHNAKTKPREEQYNSTYRVVSVLTVLHQDWL | | VCLISDFYPGAVTVAWK |
| | | NGKEYKCKVSNKALGAPIEKTISKAKGQPREPQ | | ADSSPVKAGVETTTPSK |
| | | VYTLPPSREEMTKNQVSLTCLVKGFYPSDIAVE | | QSNNKYAASSYLSLTPE |
| | | WESNGQPENNYKTTPPVLDSDGSFFLYSKLTVD | | QWKSHRSYSCQVTHEGS |
| | | KSRWQQGNVFSCSVMHEALHNHYTQKSLSLSPG | | TVEKTVAPTECS |
| | | K | | |
| A30- | 5645 | MGWSCIILFLVATATGVHSEVQLVESGGGLVKP | 5833 | MGWSCIILFLVATATGV |
| LALA | | GGSLRLSCAASGFTFSNAWMSWVRQAPGKGLEW | | HSSYELTQPPSVSVSPG |
| PGhL | | VGRIKSKTDGGTTDYAAPVKGRFTISRDDSKNT | | QTARITCSGDALPKKYA |
| 2 | | LYLQMNSLKTEDTAVYYCTTGANWGQGTLVTVS | | YWYQQKSGQAPVLVIYE |
| | | SASTKGPSVFPLAPSSKSTSGGTAALGCLVKDY | | DSKRPSGIPERFSGSSS |
| | | FPEPVTVSWNSGALTSGVHTFPAVLQSSGLYSL | | GTMATLTISGAQVEDEA |
| | | SSVVTVPSSSLGTQTYICNVNHKPSNTKVDKKV | | DYYCYSTDSSGNHWVFG |
| | | EPKSCDKTHTCPPCPAPEAAGGPSVFLFPPKPK | | GGTKLTVLGQPKAAPSV |
| | | DTLMISRTPEVTCVVVDVSHEDPEVKENWYVDG | | TLFPPSSEELQANKATL |
| | | VEVHNAKTKPREEQYNSTYRVVSVLTVLHQDWL | | VCLISDFYPGAVTVAWK |
| | | NGKEYKCKVSNKALGAPIEKTISKAKGQPREPQ | | ADSSPVKAGVETTTPSK |
| | | VYTLPPSREEMTKNQVSLTCLVKGFYPSDIAVE | | QSNNKYAASSYLSLTPE |
| | | WESNGQPENNYKTTPPVLDSDGSFFLYSKLTVD | | QWKSHRSYSCQVTHEGS |
| | | KSRWQQGNVFSCSVMHEALHNHYTQKSLSLSPG | | TVEKTVAPTECS |
| | | K | | |
| A31- | 5646 | MGWSCIILFLVATATGVHSQVQLVQSGAEVKKP | 5834 | MGWSCIILFLVATATGV |
| LALA | | GASVKVSCKASGYTFTSYDINWVRQATGQGLEW | | HSQSALTQPASVSGSPG |
| PGhL | | MGWMNPNSGNTGYAQKFQGRVTMTRNTSISTAY | | QSITISCTGTSSDVGGY |
| 2 | | MELSSLRSEDTAVYYCARVAFDIWGQGTMVTVS | | NYVSWYQQHPGKAPKLM |
| | | SASTKGPSVFPLAPSSKSTSGGTAALGCLVKDY | | IYEVSNRPSGVSNRFSG |
| | | FPEPVTVSWNSGALTSGVHTFPAVLQSSGLYSL | | SKSGNTASLTISGLQAE |
| | | SSVVTVPSSSLGTQTYICNVNHKPSNTKVDKKV | | DEADYYCSSYTSSSTVF |
| | | EPKSCDKTHTCPPCPAPEAAGGPSVFLFPPKPK | | GGGTKLTVLGQPKAAPS |
| | | DTLMISRTPEVTCVVVDVSHEDPEVKFNWYVDG | | VTLFPPSSEELQANKAT |
| | | VEVHNAKTKPREEQYNSTYRVVSVLTVLHQDWL | | LVCLISDFYPGAVTVAW |
| | | NGKEYKCKVSNKALGAPIEKTISKAKGQPREPQ | | KADSSPVKAGVETTTPS |
| | | VYTLPPSREEMTKNQVSLTCLVKGFYPSDIAVE | | KQSNNKYAASSYLSLTP |
| | | WESNGQPENNYKTTPPVLDSDGSFFLYSKLTVD | | EQWKSHRSYSCQVTHEG |
| | | KSRWQQGNVFSCSVMHEALHNHYTQKSLSLSPG | | STVEKTVAPTECS |
| | | K | | |
| A32- | 5647 | MGWSCIILFLVATATGVHSQVQLVQSGAEAKKP | 5835 | MGWSCIILFLVATATGV |
| LALA | | GASVKVSCMASGYTFTTYGISWVRQAPGQGLEW | | HSQSALTQPPSASGSPG |
| PGhL | | MGWISAYNGNTKYAQKLQGRVTMTTDTSTRTAY | | QSVTISCTGTSSDVGGY |
| 2 | | MELRSLRSDDTAVYYCARQIGDYWGQGTLVTVS | | NYVSWYQQHPGKAPKLM |
| | | SASTKGPSVFPLAPSSKSTSGGTAALGCLVKDY | | IYEVIKRPSGVPDRFSG |
| | | FPEPVTVSWNSGALTSGVHTFPAVLQSSGLYSL | | SKSGNTASLTVSGLQAE |
| | | SSVVTVPSSSLGTQTYICNVNHKPSNTKVDKKV | | DEADYYCSAYAGSNNVV |
| | | EPKSCDKTHTCPPCPAPEAAGGPSVFLFPPKPK | | FGGGTQLTVLGQPKAAP |
| | | DTLMISRTPEVTCVVVDVSHEDPEVKENWYVDG | | SVTLFPPSSEELQANKA |
| | | VEVHNAKTKPREEQYNSTYRVVSVLTVLHQDWL | | TLVCLISDFYPGAVTVA |
| | | NGKEYKCKVSNKALGAPIEKTISKAKGQPREPQ | | WKADSSPVKAGVETTTP |
| | | VYTLPPSREEMTKNQVSLTCLVKGFYPSDIAVE | | SKQSNNKYAASSYLSLT |
| | | WESNGQPENNYKTTPPVLDSDGSFFLYSKLTVD | | PEQWKSHRSYSCQVTHE |
| | | KSRWQQGNVFSCSVMHEALHNHYTQKSLSLSPG | | GSTVEKTVAPTECS |
| | | K | | |
| A33- | 5648 | MGWSCIILFLVATATGVHSEVQLVESGGGLVQP | 5836 | MGWSCIILFLVATATGV |
| LALA | | GGSLRLSCAASGFTFSSYAMSWVRQAPGKGLEW | | HSQSVLTQPPSASGTPG |
| PGhL | | VSAISGSGGSTYYADSVKGRFTISRDNSKNTLY | | QRVTISCSGSSSNIGSN |
| 2 | | LQMNSLRAEDTAVYYCHQTGEDYWGQGTLVTVS | | TVNWYQQLPGTAPKLLI |
| | | SASTKGPSVFPLAPSSKSTSGGTAALGCLVKDY | | YSNNQRPSGVPDRESGS |
| | | FPEPVTVSWNSGALTSGVHTFPAVLQSSGLYSL | | KSGTSASLAISGLØSED |
| | | SSVVTVPSSSLGTQTYICNVNHKPSNTKVDKKV | | EADYYCAAWDDSLNGWV |
| | | EPKSCDKTHTCPPCPAPEAAGGPSVFLFPPKPK | | FGGGTKLTVLGQPKAAP |
| | | DTLMISRTPEVTCVVVDVSHEDPEVKENWYVDG | | SVTLFPPSSEELQANKA |
| | | VEVHNAKTKPREEQYNSTYRVVSVLTVLHQDWL | | TLVCLISDFYPGAVTVA |
| | | NGKEYKCKVSNKALGAPIEKTISKAKGQPREPQ | | WKADSSPVKAGVETTTP |
| | | VYTLPPSREEMTKNQVSLTCLVKGFYPSDIAVE | | SKQSNNKYAASSYLSLT |
| | | WESNGQPENNYKTTPPVLDSDGSFFLYSKLTVD | | PEQWKSHRSYSCQVTHE |
| | | KSRWQQGNVFSCSVMHEALHNHYTQKSLSLSPG | | GSTVEKTVAPTECS |
| | | K | | |
| A34- | 5649 | MGWSCIILFLVATATGVHSEVQLVESGGGLVKP | 5837 | MGWSCIILFLVATATGV |
| LALA | | GGSLRLSCAASGFTFSNAWMSWVRQAPGKGLEW | | HSSYELTQPPSVSVSPG |
| PGhL | | VGRIKSKSDGGTRDYAAPVKGRFTISRDDSKNT | | QTARITCSADALPKQYA |
| 2 | | LYLQMNSLKTEDTAMFYCTTGGTHWGQGTLVTV | | YWYQQKPGQAPVLVIYK |
| | | SSASTKGPSVFPLAPSSKSTSGGTAALGCLVKD | | DSERPSGIPERFSGSSS |
| | | YFPEPVTVSWNSGALTSGVHTFPAVLQSSGLYS | | GTTVTLTISGVQAEDEA |
| | | LSSVVTVPSSSLGTQTYICNVNHKPSNTKVDKK | | DYYCQSADSSATWVFGG |
| | | VEPKSCDKTHTCPPCPAPEAAGGPSVFLFPPKP | | GTKLTVLGQPKAAPSVT |
| | | KDTLMISRTPEVTCVVVDVSHEDPEVKENWYVD | | LFPPSSEELQANKATLV |
| | | GVEVHNAKTKPREEQYNSTYRVVSVLTVLHQDW | | CLISDFYPGAVTVAWKA |
| | | LNGKEYKCKVSNKALGAPIEKTISKAKGQPREP | | DSSPVKAGVETTTPSKQ |
| | | QVYTLPPSREEMTKNQVSLTCLVKGFYPSDIAV | | SNNKYAASSYLSLTPEQ |
| | | EWESNGQPENNYKTTPPVLDSDGSFFLYSKLTV | | WKSHRSYSCQVTHEGST |
| | | DKSRWQQGNVFSCSVMHEALHNHYTQKSLSLSP | | VEKTVAPTECS |
| | | GK | | |
| A35- | 5650 | MGWSCIILFLVATATGVHSQVQLVQSGAEVKKP | 5838 | MGWSCIILFLVATATGV |
| LALA | | GASVKVSCKASGYTFTSYDINWVRQATGQGLEW | | HSSYELTQPPSVSVSPG |
| PGhL | | MGWMNPNSGNTGYAQKFQGRVTMTRNTSISTAY | | QTARITCSGDALPKKYA |
| 2 | | MELSSLRSEDTAVYYCARRSFLDYWGQGTLVTV | | YWYQQKSGQAPVLVIYE |
| | | SSASTKGPSVFPLAPSSKSTSGGTAALGCLVKD | | DSKRPSGIPERFSGSSS |
| | | YFPEPVTVSWNSGALTSGVHTFPAVLQSSGLYS | | GTMATLTISGAQVEDEA |
| | | LSSVVTVPSSSLGTQTYICNVNHKPSNTKVDKK | | DYYCYSTDSSGNHRVFG |
| | | VEPKSCDKTHTCPPCPAPEAAGGPSVFLFPPKP | | GGTKLTVLGQPKAAPSV |
| | | KDTLMISRTPEVTCVVVDVSHEDPEVKFNWYVD | | TLFPPSSEELQANKATL |
| | | GVEVHNAKTKPREEQYNSTYRVVSVLTVLHQDW | | VCLISDFYPGAVTVAWK |
| | | LNGKEYKCKVSNKALGAPIEKTISKAKGQPREP | | ADSSPVKAGVETTTPSK |
| | | QVYTLPPSREEMTKNQVSLTCLVKGFYPSDIAV | | QSNNKYAASSYLSLTPE |
| | | EWESNGQPENNYKTTPPVLDSDGSFFLYSKLTV | | QWKSHRSYSCQVTHEGS |
| | | DKSRWQQGNVFSCSVMHEALHNHYTQKSLSLSP | | TVEKTVAPTECS |
| | | GK | | |
| A36- | 5651 | MGWSCIILFLVATATGVHSEVQLVESGGGLVKP | 5839 | MGWSCIILFLVATATGV |
| LALA | | GGSLRLSCAASGFTFSNAWMSWVRQAPGKGLEW | | HSSYELTQPPSVSVSPG |
| PGhL | | VGRIKSKTDGGTTDYATPVKGRFTISRADSKNT | | QTARITCSADALPKQYA |
| 2 | | LFLQMSSLKTEDTAVYFCTTGGTNWGQGTLVTV | | YWYQQKPGQAPVVVIYK |
| | | SSASTKGPSVFPLAPSSKSTSGGTAALGCLVKD | | DSERPSGIPERFSGSSS |
| | | YFPEPVTVSWNSGALTSGVHTFPAVLQSSGLYS | | GTTVTLTISGVQAEDEA |
| | | LSSVVTVPSSSLGTQTYICNVNHKPSNTKVDKK | | DYYCQSLDSSGTYWVFG |
| | | VEPKSCDKTHTCPPCPAPEAAGGPSVFLFPPKP | | GGTKLTVLGQPKAAPSV |
| | | KDTLMISRTPEVTCVVVDVSHEDPEVKENWYVD | | TLFPPSSEELQANKATL |
| | | GVEVHNAKTKPREEQYNSTYRVVSVLTVLHQDW | | VCLISDFYPGAVTVAWK |
| | | LNGKEYKCKVSNKALGAPIEKTISKAKGQPREP | | ADSSPVKAGVETTTPSK |
| | | QVYTLPPSREEMTKNQVSLTCLVKGFYPSDIAV | | QSNNKYAASSYLSLTPE |
| | | EWESNGQPENNYKTTPPVLDSDGSFFLYSKLTV | | QWKSHRSYSCQVTHEGS |
| | | DKSRWQQGNVFSCSVMHEALHNHYTQKSLSLSP | | TVEKTVAPTECS |
| | | GK | | |
| A37- | 5652 | MGWSCIILFLVATATGVHSEVQLVESGGGLVKP | 5840 | MGWSCIILFLVATATGV |
| LALA | | GGSLRLSCAASGFTFSSYSMNWVRQAPGKGLEW | | HSSYELTQPPSVSVSPG |
| PGhL | | VSSISSSSSYIYYADSVKGRFTISRDNAKNSLY | | QTASITCSGDKLGDKYA |
| 2 | | LQMNSLRAEDTAVYYCARESSGFDYWGQGTLVT | | CWYQQKPGQSPVLVIYQ |
| | | VSSASTKGPSVFPLAPSSKSTSGGTAALGCLVK | | DSKRPSGIPERFSGSNS |
| | | DYFPEPVTVSWNSGALTSGVHTFPAVLQSSGLY | | GNTATLTISGTQAMDEA |
| | | SLSSVVTVPSSSLGTQTYICNVNHKPSNTKVDK | | DYYCQAWDSSTVVFGGG |
| | | KVEPKSCDKTHTCPPCPAPEAAGGPSVFLFPPK | | TKLTVLGQPKAAPSVTL |
| | | PKDTLMISRTPEVTCVVVDVSHEDPEVKENWYV | | FPPSSEELQANKATLVC |
| | | DGVEVHNAKTKPREEQYNSTYRVVSVLTVLHQD | | LISDFYPGAVTVAWKAD |
| | | WLNGKEYKCKVSNKALGAPIEKTISKAKGQPRE | | SSPVKAGVETTTPSKQS |
| | | PQVYTLPPSREEMTKNQVSLTCLVKGFYPSDIA | | NNKYAASSYLSLTPEQW |
| | | VEWESNGQPENNYKTTPPVLDSDGSFFLYSKLT | | KSHRSYSCQVTHEGSTV |
| | | VDKSRWQQGNVFSCSVMHEALHNHYTQKSLSLS | | EKTVAPTECS |
| | | PGK | | |
| A38- | 5653 | MGWSCIILFLVATATGVHSQVQLVQSGAEVKKP | 5841 | MGWSCIILFLVATATGV |
| LALA | | GASVKVSCKASGYTFTSYDINWVRQATGQGLEW | | HSSYELTQPPSVSVSPG |
| PGhL | | MGWMNPNSGNTGYAQKFQGRVTMTRNTSISTAY | | QTASITCSGDKLGDKYA |
| 2 | | MELSSLRSEDTAVYYCARGSSWFDYWGQGTLVT | | CWYQQKPGQSPVLVIYQ |
| | | VSSASTKGPSVFPLAPSSKSTSGGTAALGCLVK | | DSKRPSGIPERFSGSNS |
| | | DYFPEPVTVSWNSGALTSGVHTFPAVLQSSGLY | | GNTATLTISGTQAMDEA |
| | | SLSSVVTVPSSSLGTQTYICNVNHKPSNTKVDK | | DYYCQAWDSSTVVFGGG |
| | | KVEPKSCDKTHTCPPCPAPEAAGGPSVFLFPPK | | TKLTVLGQPKAAPSVTL |
| | | PKDTLMISRTPEVTCVVVDVSHEDPEVKENWYV | | FPPSSEELQANKATLVC |
| | | DGVEVHNAKTKPREEQYNSTYRVVSVLTVLHQD | | LISDFYPGAVTVAWKAD |
| | | WLNGKEYKCKVSNKALGAPIEKTISKAKGQPRE | | SSPVKAGVETTTPSKQS |
| | | PQVYTLPPSREEMTKNQVSLTCLVKGFYPSDIA | | NNKYAASSYLSLTPEQW |
| | | VEWESNGQPENNYKTTPPVLDSDGSFFLYSKLT | | KSHRSYSCQVTHEGSTV |
| | | VDKSRWQQGNVFSCSVMHEALHNHYTQKSLSLS | | EKTVAPTECS |
| | | PGK | | |
| A39- | 5654 | MGWSCIILFLVATATGVHSEVQLVESGGGLVKP | 5842 | MGWSCIILFLVATATGV |
| LALA | | GGSLRLSCAASGFTFSNAWMSWVRQAPGKGLEW | | HSSYELTQPPSVSVSPG |
| PGhL | | VGRIKSKTDGGTTDYAAPVKGRFTISRDDSKNT | | QTASITCSGDKLGDKYA |
| 2 | | LYLQMNSLKTEDTAVYYCTLNWGDYWGQGTLVT | | CWYQQKPGQSPVLVIYQ |
| | | VSSASTKGPSVFPLAPSSKSTSGGTAALGCLVK | | DSKRPSGIPERFSGSNS |
| | | DYFPEPVTVSWNSGALTSGVHTFPAVLQSSGLY | | GNTATLTISGTQAMDEA |
| | | SLSSVVTVPSSSLGTQTYICNVNHKPSNTKVDK | | DYYCQAWDSSTVVFGGG |
| | | KVEPKSCDKTHTCPPCPAPEAAGGPSVFLFPPK | | TKLTVLGQPKAAPSVTL |
| | | PKDTLMISRTPEVTCVVVDVSHEDPEVKENWYV | | FPPSSEELQANKATLVC |
| | | DGVEVHNAKTKPREEQYNSTYRVVSVLTVLHQD | | LISDFYPGAVTVAWKAD |
| | | WLNGKEYKCKVSNKALGAPIEKTISKAKGQPRE | | SSPVKAGVETTTPSKQS |
| | | PQVYTLPPSREEMTKNQVSLTCLVKGFYPSDIA | | NNKYAASSYLSLTPEQW |
| | | VEWESNGQPENNYKTTPPVLDSDGSFFLYSKLT | | KSHRSYSCQVTHEGSTV |
| | | VDKSRWQQGNVFSCSVMHEALHNHYTQKSLSLS | | EKTVAPTECS |
| | | PGK | | |
| A40- | 5655 | MGWSCIILFLVATATGVHSEVQLVESGGGLVKP | 5843 | MGWSCIILFLVATATGV |
| LALA | | GGSLRLSCAASGFTFSSYSMNWVRQAPGKGLEW | | HSSSELTQDPAVSVALG |
| PGhL | | VSSISSSSSYIYYADSVKGRFTISRDNAKNSLY | | QTVRITCQGDSLRSYYA |
| 2 | | LQMNSLRAEDTAVYYCARAADAFDIWGQGTMVT | | SWYQQKPGQAPVLVIYG |
| | | VSSASTKGPSVFPLAPSSKSTSGGTAALGCLVK | | KNNRPSGIPDRFSGSSS |
| | | DYFPEPVTVSWNSGALTSGVHTFPAVLQSSGLY | | GNTASLTITGAQAEDEA |
| | | SLSSVVTVPSSSLGTQTYICNVNHKPSNTKVDK | | DYYCNSRDSSGNHWVFG |
| | | KVEPKSCDKTHTCPPCPAPEAAGGPSVFLFPPK | | GGTKLTVLGQPKAAPSV |
| | | PKDTLMISRTPEVTCVVVDVSHEDPEVKFNWYV | | TLFPPSSEELQANKATL |
| | | DGVEVHNAKTKPREEQYNSTYRVVSVLTVLHQD | | VCLISDFYPGAVTVAWK |
| | | WLNGKEYKCKVSNKALGAPIEKTISKAKGQPRE | | ADSSPVKAGVETTTPSK |
| | | PQVYTLPPSREEMTKNQVSLTCLVKGFYPSDIA | | QSNNKYAASSYLSLTPE |
| | | VEWESNGQPENNYKTTPPVLDSDGSFFLYSKLT | | QWKSHRSYSCQVTHEGS |
| | | VDKSRWQQGNVFSCSVMHEALHNHYTQKSLSLS | | TVEKTVAPTECS |
| | | PGK | | |
| A41- | 5656 | MGWSCIILFLVATATGVHSEVQLVESGGGLVQP | 5844 | MGWSCIILFLVATATGV |
| LALA | | GGSLRLSCAASGFTFSSYDMHWVRQATGKGLEW | | HSQSALTQPASVSGSPG |
| PGhL | | VSAIGTAGDTYYPGSVKGRFTISRENAKNSLYL | | QSITISCTGTSSDVGGY |
| 2 | | QMNSLRAGDTAVYYCARGGSDAFDIWGQGTMVT | | NYVSWYQQHPGKAPKLM |
| | | VSSASTKGPSVFPLAPSSKSTSGGTAALGCLVK | | IYDVSKRPSGVSNRFSG |
| | | DYFPEPVTVSWNSGALTSGVHTFPAVLQSSGLY | | SKSGNTASLTISGLQAE |
| | | SLSSVVTVPSSSLGTQTYICNVNHKPSNTKVDK | | DEADYYCCSYAGSVVFG |
| | | KVEPKSCDKTHTCPPCPAPEAAGGPSVFLFPPK | | GGTKLTVLGQPKAAPSV |
| | | PKDTLMISRTPEVTCVVVDVSHEDPEVKENWYV | | TLFPPSSEELQANKATL |
| | | DGVEVHNAKTKPREEQYNSTYRVVSVLTVLHQD | | VCLISDFYPGAVTVAWK |
| | | WLNGKEYKCKVSNKALGAPIEKTISKAKGQPRE | | ADSSPVKAGVETTTPSK |
| | | PQVYTLPPSREEMTKNQVSLTCLVKGFYPSDIA | | QSNNKYAASSYLSLTPE |
| | | VEWESNGQPENNYKTTPPVLDSDGSFFLYSKLT | | QWKSHRSYSCQVTHEGS |
| | | VDKSRWQQGNVFSCSVMHEALHNHYTQKSLSLS | | TVEKTVAPTECS |
| | | PGK | | |
| A42- | 5657 | MGWSCIILFLVATATGVHSEVQLVESGGGLVKP | 5845 | MGWSCIILFLVATATGV |
| LALA | | GGSLRLSCTASEFTERNAWMIWVRQAPGKGLEW | | HSSSELTQDPAVSVALG |
| PGhL | | VGRIRSEIDGGTTDYAAPVKGRFTISRDDSKDT | | QTVRITCQGDSLRSYYA |
| 2 | | LYLYMNSLKVEDTAVYYCTTDHPYYWGHGTLVT | | SWYQQKPGQAPVLVIYG |
| | | VSSASTKGPSVFPLAPSSKSTSGGTAALGCLVK | | KNNRPSGIPDRFSGSSS |
| | | DYFPEPVTVSWNSGALTSGVHTFPAVLQSSGLY | | GNTASLTITGAQAEDEA |
| | | SLSSVVTVPSSSLGTQTYICNVNHKPSNTKVDK | | DYYCNSRDSSGNHVVFG |
| | | KVEPKSCDKTHTCPPCPAPEAAGGPSVFLFPPK | | GGTKLTVLGQPKAAPSV |
| | | PKDTLMISRTPEVTCVVVDVSHEDPEVKENWYV | | TLFPPSSEELQANKATL |
| | | DGVEVHNAKTKPREEQYNSTYRVVSVLTVLHQD | | VCLISDFYPGAVTVAWK |
| | | WLNGKEYKCKVSNKALGAPIEKTISKAKGQPRE | | ADSSPVKAGVETTTPSK |
| | | PQVYTLPPSREEMTKNQVSLTCLVKGFYPSDIA | | QSNNKYAASSYLSLTPE |
| | | VEWESNGQPENNYKTTPPVLDSDGSFFLYSKLT | | QWKSHRSYSCQVTHEGS |
| | | VDKSRWQQGNVFSCSVMHEALHNHYTQKSLSLS | | TVEKTVAPTECS |
| | | PGK | | |
| A43- | 5658 | MGWSCIILFLVATATGVHSEVQLVESGGGLVKP | 5846 | MGWSCIILFLVATATGV |
| LALA | | GGSLRLSCAASGFTFSNAWMSWVRQAPGKGLEW | | HSSSELTQDPAVSVALG |
| PGhL | | VGRIKSKTDGGTTDFTAPVKGRFTISRDDSKNT | | QTVRITCQGDSLRSYYA |
| 2 | | LYLQMNSLKTEDTAVYYCTTDHPYYWGQGTLVT | | SWYQQKPGQAPVLVIYG |
| | | VSSASTKGPSVFPLAPSSKSTSGGTAALGCLVK | | KNNRPSGIPDRFSGSSS |
| | | DYFPEPVTVSWNSGALTSGVHTFPAVLQSSGLY | | GNTASLTITGAQAEDEA |
| | | SLSSVVTVPSSSLGTQTYICNVNHKPSNTKVDK | | DYYCNSRDSSGNHWVFG |
| | | KVEPKSCDKTHTCPPCPAPEAAGGPSVFLFPPK | | GGTKLTVLGQPKAAPSV |
| | | PKDTLMISRTPEVTCVVVDVSHEDPEVKENWYV | | TLFPPSSEELQANKATL |
| | | DGVEVHNAKTKPREEQYNSTYRVVSVLTVLHQD | | VCLISDFYPGAVTVAWK |
| | | WLNGKEYKCKVSNKALGAPIEKTISKAKGQPRE | | ADSSPVKAGVETTTPSK |
| | | PQVYTLPPSREEMTKNQVSLTCLVKGFYPSDIA | | QSNNKYAASSYLSLTPE |
| | | VEWESNGQPENNYKTTPPVLDSDGSFFLYSKLT | | QWKSHRSYSCQVTHEGS |
| | | VDKSRWQQGNVFSCSVMHEALHNHYTQKSLSLS | | TVEKTVAPTECS |
| | | PGK | | |
| A44- | 5659 | MGWSCIILFLVATATGVHSQVQLVESGGGVVQP | 5847 | MGWSCIILFLVATATGV |
| LALA | | GRSLRLSCAASGFTFSNYGIHWVRQAPGKGLEW | | HSQSVLTQPPSVSEAPR |
| PGhL | | VAVIWYDGSNEYYVDSVKGRFIISRDNSKNTLY | | QRVTISCSGSSSNIGNN |
| 2 | | LQMNSLRAEDTALYYCALAVTGFDYWGQGTLVT | | AVNWYQQLPGKAPKLLI |
| | | VSSASTKGPSVFPLAPSSKSTSGGTAALGCLVK | | YYDDLLPSGVSDRFSGS |
| | | DYFPEPVTVSWNSGALTSGVHTFPAVLQSSGLY | | KSGTSASLAISGLQSED |
| | | SLSSVVTVPSSSLGTQTYICNVNHKPSNTKVDK | | EADYYCAAWDDRLNGPV |
| | | KVEPKSCDKTHTCPPCPAPEAAGGPSVELFPPK | | FGGGTKLTVLGQPKAAP |
| | | PKDTLMISRTPEVTCVVVDVSHEDPEVKENWYV | | SVTLFPPSSEELQANKA |
| | | DGVEVHNAKTKPREEQYNSTYRVVSVLTVLHQD | | TLVCLISDFYPGAVTVA |
| | | WLNGKEYKCKVSNKALGAPIEKTISKAKGQPRE | | WKADSSPVKAGVETTTP |
| | | PQVYTLPPSREEMTKNQVSLTCLVKGFYPSDIA | | SKQSNNKYAASSYLSLT |
| | | VEWESNGQPENNYKTTPPVLDSDGSFFLYSKLT | | PEQWKSHRSYSCQVTHE |
| | | VDKSRWQQGNVFSCSVMHEALHNHYTQKSLSLS | | GSTVEKTVAPTECS |
| | | PGK | | |
| A45- | 5660 | MGWSCIILFLVATATGVHSQVQLVESGGGLVKP | 5848 | MGWSCIILFLVATATGV |
| LALA | | GGSLRLSCAASGFTFSDYYMSWIRQAPGKGLEW | | HSQSALTQPASVSGSPG |
| PGhL | | VSYISSSGSTIYYPDSVKGRFTISRDNAKNSLY | | QSITISCTGTSSDVGGY |
| 2 | | LQMNSLRAEDTAVYYCARDGAAFDIWGQGTMVT | | NYVSWYQQHPGKAPKLM |
| | | VSSASTKGPSVFPLAPSSKSTSGGTAALGCLVK | | IYDVSKRPSGVSNRFSG |
| | | DYFPEPVTVSWNSGALTSGVHTFPAVLQSSGLY | | SKSGNTASLTISGLQAE |
| | | SLSSVVTVPSSSLGTQTYICNVNHKPSNTKVDK | | DETDYYCCSYAGSSTLV |
| | | KVEPKSCDKTHTCPPCPAPEAAGGPSVFLFPPK | | FGGGTKLTVLGQPKAAP |
| | | PKDTLMISRTPEVTCVVVDVSHEDPEVKENWYV | | SVTLFPPSSEELQANKA |
| | | DGVEVHNAKTKPREEQYNSTYRVVSVLTVLHQD | | TLVCLISDFYPGAVTVA |
| | | WLNGKEYKCKVSNKALGAPIEKTISKAKGQPRE | | WKADSSPVKAGVETTTP |
| | | PQVYTLPPSREEMTKNQVSLTCLVKGFYPSDIA | | SKQSNNKYAASSYLSLT |
| | | VEWESNGQPENNYKTTPPVLDSDGSFFLYSKLT | | PEQWKSHRSYSCQVTHE |
| | | VDKSRWQQGNVFSCSVMHEALHNHYTQKSLSLS | | GSTVEKTVAPTECS |
| | | PGK | | |
| A46- | 5661 | MGWSCIILFLVATATGVHSQVQLVQSGAEMKKP | 5849 | MGWSCIILFLVATATGV |
| LALA | | GASVKVSCKASGYTFTNYGISWVRQAPGQGLEW | | HSSYELTQPPSVSVSPG |
| PGhL | | MGWINTYNDKTNFALKVQGRVTMTTDTSTSTAY | | QTASITCSGDKLGDKHA |
| 2 | | MELRSLRSDDTAVYYCARDRGYSFDYWGQGTLV | | CWYQQKPGQSPMLVIYQ |
| | | TVSSASTKGPSVFPLAPSSKSTSGGTAALGCLV | | DSKRPSGIPERFSGSNS |
| | | KDYFPEPVTVSWNSGALTSGVHTFPAVLQSSGL | | GNTATLTISGTQPMDEA |
| | | YSLSSVVTVPSSSLGTQTYICNVNHKPSNTKVD | | DYYCQAWDSSTFGGGTK |
| | | KKVEPKSCDKTHTCPPCPAPEAAGGPSVELFPP | | LTVLGQPKAAPSVTLFP |
| | | KPKDTLMISRTPEVTCVVVDVSHEDPEVKENWY | | PSSEELQANKATLVCLI |
| | | VDGVEVHNAKTKPREEQYNSTYRVVSVLTVLHQ | | SDFYPGAVTVAWKADSS |
| | | DWLNGKEYKCKVSNKALGAPIEKTISKAKGQPR | | PVKAGVETTTPSKQSNN |
| | | EPQVYTLPPSREEMTKNQVSLTCLVKGFYPSDI | | KYAASSYLSLTPEQWKS |
| | | AVEWESNGQPENNYKTTPPVLDSDGSFFLYSKL | | HRSYSCQVTHEGSTVEK |
| | | TVDKSRWQQGNVFSCSVMHEALHNHYTQKSLSL | | TVAPTECS |
| | | SPGK | | |
| A47- | 5662 | MGWSCIILFLVATATGVHSQVQLVQSGAEVKKP | 5850 | MGWSCIILFLVATATGV |
| LALA | | GASVKVSCKASGYTFTSYGISWVRQAPGQGLEW | | HSSYELTQPSSVSVSPG |
| PGhL | | MGWISAYNGNTNYAQRLQGRVTMTTDTSTSTAY | | QTARITCSGDVLAKKYA |
| 2 | | MELRSLISDDTAVYYCARNHYYYLDVWGKGTTV | | RWFQQKPGQAPVLVIYK |
| | | TVSSASTKGPSVFPLAPSSKSTSGGTAALGCLV | | DSERPSGIPERFSGSSS |
| | | KDYFPEPVTVSWNSGALTSGVHTFPAVLQSSGL | | GTTVTLTISGAQVEDEA |
| | | YSLSSVVTVPSSSLGTQTYICNVNHKPSNTKVD | | DYYCYSAADNNRVFGGG |
| | | KKVEPKSCDKTHTCPPCPAPEAAGGPSVFLFPP | | TKLTVLGQPKAAPSVTL |
| | | KPKDTLMISRTPEVTCVVVDVSHEDPEVKENWY | | FPPSSEELQANKATLVC |
| | | VDGVEVHNAKTKPREEQYNSTYRVVSVLTVLHQ | | LISDFYPGAVTVAWKAD |
| | | DWLNGKEYKCKVSNKALGAPIEKTISKAKGQPR | | SSPVKAGVETTTPSKQS |
| | | EPQVYTLPPSREEMTKNQVSLTCLVKGFYPSDI | | NNKYAASSYLSLTPEQW |
| | | AVEWESNGQPENNYKTTPPVLDSDGSFFLYSKL | | KSHRSYSCQVTHEGSTV |
| | | TVDKSRWQQGNVFSCSVMHEALHNHYTQKSLSL | | EKTVAPTECS |
| | | SPGK | | |
| A48- | 5663 | MGWSCIILFLVATATGVHSEVQLVESGGGLVQP | 5851 | MGWSCIILFLVATATGV |
| LALA | | GGSLRLSCAASGFTFSSYDMHWVRQATGKGLEW | | HSSYELTQPSSVSVSPG |
| PGhL | | VSAIGTAGDTYYPGSVKGRFTISRENAKNSLYL | | QTARITCSGDVLAKKYA |
| 2 | | QMNSLRAGDTAVYYCARVSPTGTTDYWGQGTLV | | RWFQQKPGQAPVLVIYK |
| | | TVSSASTKGPSVFPLAPSSKSTSGGTAALGCLV | | DSERPSGIPERFSGSSS |
| | | KDYFPEPVTVSWNSGALTSGVHTFPAVLQSSGL | | GTTVTLTISGAQVEDEA |
| | | YSLSSVVTVPSSSLGTQTYICNVNHKPSNTKVD | | DYYCYSAADNNLVFGGG |
| | | KKVEPKSCDKTHTCPPCPAPEAAGGPSVFLFPP | | TKLTVLGQPKAAPSVTL |
| | | KPKDTLMISRTPEVTCVVVDVSHEDPEVKENWY | | FPPSSEELQANKATLVC |
| | | VDGVEVHNAKTKPREEQYNSTYRVVSVLTVLHQ | | LISDFYPGAVTVAWKAD |
| | | DWLNGKEYKCKVSNKALGAPIEKTISKAKGQPR | | SSPVKAGVETTTPSKQS |
| | | EPQVYTLPPSREEMTKNQVSLTCLVKGFYPSDI | | NNKYAASSYLSLTPEQW |
| | | AVEWESNGQPENNYKTTPPVLDSDGSFFLYSKL | | KSHRSYSCQVTHEGSTV |
| | | TVDKSRWQQGNVFSCSVMHEALHNHYTQKSLSL | | EKTVAPTECS |
| | | SPGK | | |
| A49- | 5664 | MGWSCIILFLVATATGVHSEVQLVESGGGLVKP | 5852 | MGWSCIILFLVATATGV |
| LALA | | GGSLRLSCAASGFTFSNAWMSWVRQAPGKGLEW | | HSSYELTQPSSVSVSPG |
| PGhL | | VGRIKSKTDGGTTDYAAPVKGRFTISRDDSKNT | | QTARITCSGDVLAKKYA |
| 2 | | LYLQMNSLKTEDTAVYYCTARPLGDVWGKGTTV | | RWFQQKPGQAPVLVIYK |
| | | TVSSASTKGPSVFPLAPSSKSTSGGTAALGCLV | | DSERPSGIPERFSGSSS |
| | | KDYFPEPVTVSWNSGALTSGVHTFPAVLQSSGL | | GTTVTLTISGAQVEDEA |
| | | YSLSSVVTVPSSSLGTQTYICNVNHKPSNTKVD | | DYYCYSAADNNYVFGTG |
| | | KKVEPKSCDKTHTCPPCPAPEAAGGPSVFLFPP | | TKVTVLGQPKAAPSVTL |
| | | KPKDTLMISRTPEVTCVVVDVSHEDPEVKENWY | | FPPSSEELQANKATLVC |
| | | VDGVEVHNAKTKPREEQYNSTYRVVSVLTVLHQ | | LISDFYPGAVTVAWKAD |
| | | DWLNGKEYKCKVSNKALGAPIEKTISKAKGQPR | | SSPVKAGVETTTPSKQS |
| | | EPQVYTLPPSREEMTKNQVSLTCLVKGFYPSDI | | NNKYAASSYLSLTPEQW |
| | | AVEWESNGQPENNYKTTPPVLDSDGSFFLYSKL | | KSHRSYSCQVTHEGSTV |
| | | TVDKSRWQQGNVFSCSVMHEALHNHYTQKSLSL | | EKTVAPTECS |
| | | SPGK | | |
| A50- | 5665 | MGWSCIILFLVATATGVHSEVQLVESGGGLVKP | 5853 | MGWSCIILFLVATATGV |
| LALA | | GGSLRLSCAASGFTFSNAWMSWVRQAPGKGLEW | | HSSYELTQPSSVSVSPG |
| PGhL | | VGRIKSKTDGGTTDYAAPVKGRFTISRDDSKNT | | QTARITCSGDVLAKKYA |
| 2 | | LYLQMNSLKTEDTAVYYCTTDNGFDYWGQGTLV | | RWFQQKPGQAPVLVIYK |
| | | TVSSASTKGPSVFPLAPSSKSTSGGTAALGCLV | | DSERPSGIPERFSGSSS |
| | | KDYFPEPVTVSWNSGALTSGVHTFPAVLQSSGL | | GTTVTLTISGAQVEDEA |
| | | YSLSSVVTVPSSSLGTQTYICNVNHKPSNTKVD | | DYYCYSAADNNLVFGGG |
| | | KKVEPKSCDKTHTCPPCPAPEAAGGPSVFLFPP | | TKLTVLGQPKAAPSVTL |
| | | KPKDTLMISRTPEVTCVVVDVSHEDPEVKENWY | | FPPSSEELQANKATLVC |
| | | VDGVEVHNAKTKPREEQYNSTYRVVSVLTVLHQ | | LISDFYPGAVTVAWKAD |
| | | DWLNGKEYKCKVSNKALGAPIEKTISKAKGQPR | | SSPVKAGVETTTPSKQS |
| | | EPQVYTLPPSREEMTKNQVSLTCLVKGFYPSDI | | NNKYAASSYLSLTPEQW |
| | | AVEWESNGQPENNYKTTPPVLDSDGSFFLYSKL | | KSHRSYSCQVTHEGSTV |
| | | TVDKSRWQQGNVFSCSVMHEALHNHYTQKSLSL | | EKTVAPTECS |
| | | SPGK | | |
| A51- | 5666 | MGWSCIILFLVATATGVHSEVQLVESGGGLVKP | 5854 | MGWSCIILFLVATATGV |
| LALA | | GGSLRLSCAASGFTFSSYSMNWVRQAPGKGLEW | | HSSSELTQDPAVSVALG |
| PGhL | | VSSISSSSSYIYYADSVKGRFTISRDNAKNSLY | | QTVRITCQGDRLRSYYA |
| 2 | | LQMNSLRAEDTAVYYCARDLISSEDYWGQGTLV | | SWYQQKPGQAPVLVIYG |
| | | TVSSASTKGPSVFPLAPSSKSTSGGTAALGCLV | | KNNRPSGIPDRFSGSSS |
| | | KDYFPEPVTVSWNSGALTSGVHTFPAVLQSSGL | | GNTASLTITGAQAEDEA |
| | | YSLSSVVTVPSSSLGTQTYICNVNHKPSNTKVD | | DYHCNSRDSSGNHLVFG |
| | | KKVEPKSCDKTHTCPPCPAPEAAGGPSVFLFPP | | GGTKLTVLGQPKAAPSV |
| | | KPKDTLMISRTPEVTCVVVDVSHEDPEVKENWY | | TLFPPSSEELQANKATL |
| | | VDGVEVHNAKTKPREEQYNSTYRVVSVLTVLHQ | | VCLISDFYPGAVTVAWK |
| | | DWLNGKEYKCKVSNKALGAPIEKTISKAKGQPR | | ADSSPVKAGVETTTPSK |
| | | EPQVYTLPPSREEMTKNQVSLTCLVKGFYPSDI | | QSNNKYAASSYLSLTPE |
| | | AVEWESNGQPENNYKTTPPVLDSDGSFFLYSKL | | QWKSHRSYSCQVTHEGS |
| | | TVDKSRWQQGNVFSCSVMHEALHNHYTQKSLSL | | TVEKTVAPTECS |
| | | SPGK | | |
| A52- | 5667 | MGWSCIILFLVATATGVHSEVQLVESGGGLVQP | 5855 | MGWSCIILFLVATATGV |
| LALA | | GGSLRLSCAASGFTFSSYWMSWVRQAPGKGLEW | | HSSSELTQDPAVSVALG |
| PGhL | | VANIKQDGSEKYYVDSVKGRFTISRDNAKNSLY | | QTVRITCQGDSLRSYYA |
| 2 | | LQMNSLRAEDTAVYYCARRIVGAFDYWGQGTLV | | SWYQQKPGQAPVLVIYG |
| | | TVSSASTKGPSVFPLAPSSKSTSGGTAALGCLV | | KNNRPSGIPDRFSGSSS |
| | | KDYFPEPVTVSWNSGALTSGVHTFPAVLQSSGL | | GNTASLTITGAQAEDEA |
| | | YSLSSVVTVPSSSLGTQTYICNVNHKPSNTKVD | | DYYCNSRDSSGNHLVFG |
| | | KKVEPKSCDKTHTCPPCPAPEAAGGPSVFLFPP | | GGTKLTVLGQPKAAPSV |
| | | KPKDTLMISRTPEVTCVVVDVSHEDPEVKENWY | | TLFPPSSEELQANKATL |
| | | VDGVEVHNAKTKPREEQYNSTYRVVSVLTVLHQ | | VCLISDFYPGAVTVAWK |
| | | DWLNGKEYKCKVSNKALGAPIEKTISKAKGQPR | | ADSSPVKAGVETTTPSK |
| | | EPQVYTLPPSREEMTKNQVSLTCLVKGFYPSDI | | QSNNKYAASSYLSLTPE |
| | | AVEWESNGQPENNYKTTPPVLDSDGSFFLYSKL | | QWKSHRSYSCQVTHEGS |
| | | TVDKSRWQQGNVFSCSVMHEALHNHYTQKSLSL | | TVEKTVAPTECS |
| | | SPGK | | |
| A53- | 5668 | MGWSCIILFLVATATGVHSQVQLVQSGTEVKKP | 5856 | MGWSCIILFLVATATGV |
| LALA | | GASVKVSCMASRYTFTSYYIHWVRQAPGQGLEW | | HSSYVLTQPPSVSVAPG |
| PGhL | | MGIINPSGGTTGYAQKFQGRVTMTRDTSTSTVY | | KTARITCGGNNIGSKSV |
| 2 | | MELYSLRSEDTAVYYCARGGWGTMDVWGKGTTV | | HWYQQKPGQAPVLVIYY |
| | | TVSSASTKGPSVFPLAPSSKSTSGGTAALGCLV | | DSDRPSGIPERFSGSNS |
| | | KDYFPEPVTVSWNSGALTSGVHTFPAVLQSSGL | | GNTATLTISRVEAGDEA |
| | | YSLSSVVTVPSSSLGTQTYICNVNHKPSNTKVD | | DYYCQVWDSSSDHVVFG |
| | | KKVEPKSCDKTHTCPPCPAPEAAGGPSVFLFPP | | GGTKLTVLGQPKAAPSV |
| | | KPKDTLMISRTPEVTCVVVDVSHEDPEVKENWY | | TLFPPSSEELQANKATL |
| | | VDGVEVHNAKTKPREEQYNSTYRVVSVLTVLHQ | | VCLISDFYPGAVTVAWK |
| | | DWLNGKEYKCKVSNKALGAPIEKTISKAKGQPR | | ADSSPVKAGVETTTPSK |
| | | EPQVYTLPPSREEMTKNQVSLTCLVKGFYPSDI | | QSNNKYAASSYLSLTPE |
| | | AVEWESNGQPENNYKTTPPVLDSDGSFFLYSKL | | QWKSHRSYSCQVTHEGS |
| | | TVDKSRWQQGNVFSCSVMHEALHNHYTQKSLSL | | TVEKTVAPTECS |
| | | SPGK | | |
| A54- | 5669 | MGWSCIILFLVATATGVHSEVQLVESGGGLVQP | 5857 | MGWSCIILFLVATATGV |
| LALA | | GGSLRLSCAASGFTFSSYSMNWVRQAPGKGLEW | | HSSYELTQPPSVSVSPG |
| PGhL | | VSYISSSSSTIYYADSVKGRFTISRDNAKNSLY | | QTARITCSGDALPKKYA |
| 2 | | LQMNSLRDEDTAVYYCAREGWELLDYWGQGTLV | | YWYQQKSGQAPVLVIYE |
| | | TVSSASTKGPSVFPLAPSSKSTSGGTAALGCLV | | DSKRPSGIPERFSGSSS |
| | | KDYFPEPVTVSWNSGALTSGVHTFPAVLQSSGL | | GTMATLTISGAQVEDEA |
| | | YSLSSVVTVPSSSLGTQTYICNVNHKPSNTKVD | | DYYCYSTDSSGNHRVFG |
| | | KKVEPKSCDKTHTCPPCPAPEAAGGPSVFLFPP | | GGTKLTVLGQPKAAPSV |
| | | KPKDTLMISRTPEVTCVVVDVSHEDPEVKENWY | | TLFPPSSEELQANKATL |
| | | VDGVEVHNAKTKPREEQYNSTYRVVSVLTVLHQ | | VCLISDFYPGAVTVAWK |
| | | DWLNGKEYKCKVSNKALGAPIEKTISKAKGQPR | | ADSSPVKAGVETTTPSK |
| | | EPQVYTLPPSREEMTKNQVSLTCLVKGFYPSDI | | QSNNKYAASSYLSLTPE |
| | | AVEWESNGQPENNYKTTPPVLDSDGSFFLYSKL | | QWKSHRSYSCQVTHEGS |
| | | TVDKSRWQQGNVFSCSVMHEALHNHYTQKSLSL | | TVEKTVAPTECS |
| | | SPGK | | |
| A55- | 5670 | MGWSCIILFLVATATGVHSEVQLVESGGGLIQP | 5858 | MGWSCIILFLVATATGV |
| LALA | | GGSLRLSCAASGLTVSTNYMSWVRQAPGKGLEW | | HSQTVVTQEPSLTVSPG |
| PGhL | | VSVLYSGGGTYYADSVKGRFTISRDNSKNTLCL | | GTVTLTCASSTGAVTSG |
| 2 | | QMNSLRAEDTAMYYCARDNWDSYFDYWGQGTLV | | YYPNWFQQKPGQAPRAL |
| | | TVSSASTKGPSVFPLAPSSKSTSGGTAALGCLV | | IYSTSNKHSWTPARFSG |
| | | KDYFPEPVTVSWNSGALTSGVHTFPAVLQSSGL | | SLLGGKAALTLSGVQPE |
| | | YSLSSVVTVPSSSLGTQTYICNVNHKPSNTKVD | | DEAEYYCLLYYGGARVE |
| | | KKVEPKSCDKTHTCPPCPAPEAAGGPSVFLFPP | | GGGTKLTVLGQPKAAPS |
| | | KPKDTLMISRTPEVTCVVVDVSHEDPEVKENWY | | VTLFPPSSEELQANKAT |
| | | VDGVEVHNAKTKPREEQYNSTYRVVSVLTVLHQ | | LVCLISDFYPGAVTVAW |
| | | DWLNGKEYKCKVSNKALGAPIEKTISKAKGQPR | | KADSSPVKAGVETTTPS |
| | | EPQVYTLPPSREEMTKNQVSLTCLVKGFYPSDI | | KQSNNKYAASSYLSLTP |
| | | AVEWESNGQPENNYKTTPPVLDSDGSFFLYSKL | | EQWKSHRSYSCQVTHEG |
| | | TVDKSRWQQGNVFSCSVMHEALHNHYTQKSLSL | | STVEKTVAPTECS |
| | | SPGK | | |
| A56- | 5671 | MGWSCIILFLVATATGVHSEVQLVESGGGVVRP | 5859 | MGWSCIILFLVATATGV |
| LALA | | GGSLRLSCAASGFTFDDYGMSWVRQAPGKGLEW | | HSQSALTQPPSASGSPG |
| PGhL | | VSGINWNGGSTGYADSVKGRFTISRDNAKNSLY | | QSVTISCTGTSSDVGGY |
| 2 | | LQMNSLRAEDTALYYCARTTVTHMDVWGKGTTV | | NYVSWYQQHPGKAPKLM |
| | | TVSSASTKGPSVFPLAPSSKSTSGGTAALGCLV | | IYEVSKRPSGVPDRFSG |
| | | KDYFPEPVTVSWNSGALTSGVHTFPAVLQSSGL | | SKSGNTASLTVSGLQAE |
| | | YSLSSVVTVPSSSLGTQTYICNVNHKPSNTKVD | | DEADYYCSSYAGSNNLV |
| | | KKVEPKSCDKTHTCPPCPAPEAAGGPSVFLFPP | | FGGGTKLTVLGQPKAAP |
| | | KPKDTLMISRTPEVTCVVVDVSHEDPEVKENWY | | SVTLFPPSSEELQANKA |
| | | VDGVEVHNAKTKPREEQYNSTYRVVSVLTVLHQ | | TLVCLISDFYPGAVTVA |
| | | DWLNGKEYKCKVSNKALGAPIEKTISKAKGQPR | | WKADSSPVKAGVETTTP |
| | | EPQVYTLPPSREEMTKNQVSLTCLVKGFYPSDI | | SKQSNNKYAASSYLSLT |
| | | AVEWESNGQPENNYKTTPPVLDSDGSFFLYSKL | | PEQWKSHRSYSCQVTHE |
| | | TVDKSRWQQGNVFSCSVMHEALHNHYTQKSLSL | | GSTVEKTVAPTECS |
| | | SPGK | | |
| A57- | 5672 | MGWSCIILFLVATATGVHSEVQLVESGGGLIQP | 5860 | MGWSCIILFLVATATGV |
| LALA | | GGSLRLSCAASGFTVSSNYMSWVRQAPGKGLEW | | HSQSALTQPASVSGSPG |
| PGhL | | VSVIYSGGSTYYADSVKGRFTISRDNSKNTLYL | | QSITISCTGTSSDVGGY |
| 2 | | QMNSLRAEDTAVYYCARDWNYDAFDIWGQGTMV | | NYVSWYQQHPGKAPKLM |
| | | TVSSASTKGPSVFPLAPSSKSTSGGTAALGCLV | | IYDVSKRPSGVSNRFSG |
| | | KDYFPEPVTVSWNSGALTSGVHTFPAVLQSSGL | | SKSGNTASLTISGLQAE |
| | | YSLSSVVTVPSSSLGTQTYICNVNHKPSNTKVD | | DEADYYCCSYAGSSTWV |
| | | KKVEPKSCDKTHTCPPCPAPEAAGGPSVFLFPP | | FGGGTKLTVLGQPKAAP |
| | | KPKDTLMISRTPEVTCVVVDVSHEDPEVKENWY | | SVTLFPPSSEELQANKA |
| | | VDGVEVHNAKTKPREEQYNSTYRVVSVLTVLHQ | | TLVCLISDFYPGAVTVA |
| | | DWLNGKEYKCKVSNKALGAPIEKTISKAKGQPR | | WKADSSPVKAGVETTTP |
| | | EPQVYTLPPSREEMTKNQVSLTCLVKGFYPSDI | | SKQSNNKYAASSYLSLT |
| | | AVEWESNGQPENNYKTTPPVLDSDGSFFLYSKL | | PEQWKSHRSYSCQVTHE |
| | | TVDKSRWQQGNVFSCSVMHEALHNHYTQKSLSL | | GSTVEKTVAPTECS |
| | | SPGK | | |
| A58- | 5673 | MGWSCIILFLVATATGVHSEVQLVESGGGLVKP | 5861 | MGWSCIILFLVATATGV |
| LALA | | GGSLRLSCAASGFTFSSYSMNWVRQAPGKGLEW | | HSQSALTQPASVSGSPG |
| PGhL | | VSSISSSSSYIYYADSVKGRFTISRDNAKNSLY | | QSITISCTGTSSDVGGY |
| 2 | | LQMNSLRAEDTAVYYCARGDPGWFDPWGQGTLV | | NYVSWYQQHPGKAPKLM |
| | | TVSSASTKGPSVFPLAPSSKSTSGGTAALGCLV | | IYDVSKRPSGVSNRFSG |
| | | KDYFPEPVTVSWNSGALTSGVHTFPAVLQSSGL | | SKSGNTASLTISGLQAE |
| | | YSLSSVVTVPSSSLGTQTYICNVNHKPSNTKVD | | DEADYYCCSYAGSSTFW |
| | | KKVEPKSCDKTHTCPPCPAPEAAGGPSVFLFPP | | VFGGGTKLTVLGQPKAA |
| | | KPKDTLMISRTPEVTCVVVDVSHEDPEVKENWY | | PSVTLFPPSSEELQANK |
| | | VDGVEVHNAKTKPREEQYNSTYRVVSVLTVLHQ | | ATLVCLISDFYPGAVTV |
| | | DWLNGKEYKCKVSNKALGAPIEKTISKAKGQPR | | AWKADSSPVKAGVETTT |
| | | EPQVYTLPPSREEMTKNQVSLTCLVKGFYPSDI | | PSKQSNNKYAASSYLSL |
| | | AVEWESNGQPENNYKTTPPVLDSDGSFFLYSKL | | TPEQWKSHRSYSCQVTH |
| | | TVDKSRWQQGNVFSCSVMHEALHNHYTQKSLSL | | EGSTVEKTVAPTECS |
| | | SPGK | | |
| A59- | 5674 | MGWSCIILFLVATATGVHSEVQLVESGGGLVQP | 5862 | MGWSCIILFLVATATGV |
| LALA | | GGSLRLSCAASGFTFSSYWMHWVRQAPGKGLVW | | HSSYELTQPPSVSVSPG |
| PGhL | | VSRINSDGSSTSYADSVKGRFTISRDNAKNTLY | | QTASITCSGDKLGDKYA |
| 2 | | LQMNSLRAEDTAVYYCARENWNYWFDPWGQGTL | | CWYQQKPGQSPVLVIYQ |
| | | VTVSSASTKGPSVFPLAPSSKSTSGGTAALGCL | | DSKRPSGIPERFSGSNS |
| | | VKDYFPEPVTVSWNSGALTSGVHTFPAVLQSSG | | GNTATLTISGTQAMDEA |
| | | LYSLSSVVTVPSSSLGTQTYICNVNHKPSNTKV | | DYYCQAWDSSTVVFGGG |
| | | DKKVEPKSCDKTHTCPPCPAPEAAGGPSVFLFP | | TKLTVLGQPKAAPSVTL |
| | | PKPKDTLMISRTPEVTCVVVDVSHEDPEVKENW | | FPPSSEELQANKATLVC |
| | | YVDGVEVHNAKTKPREEQYNSTYRVVSVLTVLH | | LISDFYPGAVTVAWKAD |
| | | QDWLNGKEYKCKVSNKALGAPIEKTISKAKGQP | | SSPVKAGVETTTPSKQS |
| | | REPQVYTLPPSREEMTKNQVSLTCLVKGFYPSD | | NNKYAASSYLSLTPEQW |
| | | IAVEWESNGQPENNYKTTPPVLDSDGSFFLYSK | | KSHRSYSCQVTHEGSTV |
| | | LTVDKSRWQQGNVFSCSVMHEALHNHYTQKSLS | | EKTVAPTECS |
| | | LSPGK | | |
| A60- | 5675 | MGWSCIILFLVATATGVHSQVQLQESGPGLVKP | 5863 | MGWSCIILFLVATATGV |
| LALA | | SGTLSLTCAVSGGSISSNNWWSWVRQPPGKGLE | | HSSYELTQPPSVSVSPG |
| PGhL | | WIGEIYHSGSTNYNPSLKSRVTISVDKSKNQFS | | QTASITCSGDKLGDKYA |
| 2 | | LKVNSVTAADTAIFYCARLRPGDSFDYWGLGTL | | CWYQQRPGQSPVLVIYQ |
| | | VTVSSASTKGPSVFPLAPSSKSTSGGTAALGCL | | DNKRPSGIPERFSGSNS |
| | | VKDYFPEPVTVSWNSGALTSGVHTFPAVLQSSG | | GNTATLTISGTQAMDEA |
| | | LYSLSSVVTVPSSSLGTQTYICNVNHKPSNTKV | | DYYCQAWDSSTALVFGG |
| | | DKKVEPKSCDKTHTCPPCPAPEAAGGPSVFLFP | | GTKLTVLGQPKAAPSVT |
| | | PKPKDTLMISRTPEVTCVVVDVSHEDPEVKENW | | LFPPSSEELQANKATLV |
| | | YVDGVEVHNAKTKPREEQYNSTYRVVSVLTVLH | | CLISDFYPGAVTVAWKA |
| | | QDWLNGKEYKCKVSNKALGAPIEKTISKAKGQP | | DSSPVKAGVETTTPSKQ |
| | | REPQVYTLPPSREEMTKNQVSLTCLVKGFYPSD | | SNNKYAASSYLSLTPEQ |
| | | IAVEWESNGQPENNYKTTPPVLDSDGSFFLYSK | | WKSHRSYSCQVTHEGST |
| | | LTVDKSRWQQGNVFSCSVMHEALHNHYTQKSLS | | VEKTVAPTECS |
| | | LSPGK | | |
| A61- | 5676 | MGWSCIILFLVATATGVHSQVQLVQSGSELKKP | 5864 | MGWSCIILFLVATATGV |
| LALA | | GASVKVSCKASGYTFTRNGLNWVRQAPGQGLEW | | HSSYELTQPPSVSVSPG |
| PGhL | | MGWINTNIGNPTYAQGFTGRFVFSLDTSVSTAY | | QTASITCSGDKLGDKYA |
| 2 | | LQISRLQAEDTAVYYCARSPNWGLFDYWGQGTL | | CWYQQKPGQSPVLVIYQ |
| | | VTVSSASTKGPSVFPLAPSSKSTSGGTAALGCL | | DSKRPSGIPERFSGSNS |
| | | VKDYFPEPVTVSWNSGALTSGVHTFPAVLQSSG | | GNTATLTISGTQAMDEA |
| | | LYSLSSVVTVPSSSLGTQTYICNVNHKPSNTKV | | DYYCQAWDSSTSGVFGG |
| | | DKKVEPKSCDKTHTCPPCPAPEAAGGPSVFLFP | | GTKLTVLGQPKAAPSVT |
| | | PKPKDTLMISRTPEVTCVVVDVSHEDPEVKENW | | LFPPSSEELQANKATLV |
| | | YVDGVEVHNAKTKPREEQYNSTYRVVSVLTVLH | | CLISDFYPGAVTVAWKA |
| | | QDWLNGKEYKCKVSNKALGAPIEKTISKAKGQP | | DSSPVKAGVETTTPSKQ |
| | | REPQVYTLPPSREEMTKNQVSLTCLVKGFYPSD | | SNNKYAASSYLSLTPEQ |
| | | IAVEWESNGQPENNYKTTPPVLDSDGSFFLYSK | | WKSHRSYSCQVTHEGST |
| | | LTVDKSRWQQGNVFSCSVMHEALHNHYTQKSLS | | VEKTVAPTECS |
| | | LSPGK | | |
| A62- | 5677 | MGWSCIILFLVATATGVHSEVQLVESGGGLVKP | 5865 | MGWSCIILFLVATATGV |
| LALA | | GGSLRLSCAASGFTFSSYSMNWVRQAPGKGLEW | | HSSSELTQDPAVSVALG |
| PGhL | | VSSISSSSSYIYYADSVKGRFTISRDNAKNSLY | | QTVRITCQGDSLRSYYA |
| 2 | | LQMNSLRAEDTAVYYCARDRGATGFDYWGQGTL | | SWYQQKPGQAPVLVIYG |
| | | VTVSSASTKGPSVFPLAPSSKSTSGGTAALGCL | | KNNRPSGIPDRFSGSSS |
| | | VKDYFPEPVTVSWNSGALTSGVHTFPAVLQSSG | | GNTASLTITGAQAEDEA |
| | | LYSLSSVVTVPSSSLGTQTYICNVNHKPSNTKV | | DYYCNSRDSSGNHWVFG |
| | | DKKVEPKSCDKTHTCPPCPAPEAAGGPSVFLFP | | GGTKLTVLGQPKAAPSV |
| | | PKPKDTLMISRTPEVTCVVVDVSHEDPEVKENW | | TLFPPSSEELQANKATL |
| | | YVDGVEVHNAKTKPREEQYNSTYRVVSVLTVLH | | VCLISDFYPGAVTVAWK |
| | | QDWLNGKEYKCKVSNKALGAPIEKTISKAKGQP | | ADSSPVKAGVETTTPSK |
| | | REPQVYTLPPSREEMTKNQVSLTCLVKGFYPSD | | QSNNKYAASSYLSLTPE |
| | | IAVEWESNGQPENNYKTTPPVLDSDGSFFLYSK | | QWKSHRSYSCQVTHEGS |
| | | LTVDKSRWQQGNVFSCSVMHEALHNHYTQKSLS | | TVEKTVAPTECS |
| | | LSPGK | | |
| A63- | 5678 | MGWSCIILFLVATATGVHSQVQLVQSGAEVKKP | 5866 | MGWSCIILFLVATATGV |
| LALA | | GASVKVSCKASGYTFTSYGISWVRQAPGQGLEW | | HSSYELTQPPSVSVSPG |
| PGhL | | MGWISAYNGNTNYAQKLQGRVTMTTDTSTSTAY | | QTARITCSGDALPKKYA |
| 2 | | MELRSLRSDDTAVYYCARESGELLGDYWGQGTL | | YWYQQKSGQAPVLVIYE |
| | | VTVSSASTKGPSVFPLAPSSKSTSGGTAALGCL | | DSKRPSGIPERFSGSSS |
| | | VKDYFPEPVTVSWNSGALTSGVHTFPAVLQSSG | | GTMATLTISGAQVEDEA |
| | | LYSLSSVVTVPSSSLGTQTYICNVNHKPSNTKV | | DYYCYSTDSSGNHRVFG |
| | | DKKVEPKSCDKTHTCPPCPAPEAAGGPSVFLFP | | GGTKLTVLGQPKAAPSV |
| | | PKPKDTLMISRTPEVTCVVVDVSHEDPEVKENW | | TLFPPSSEELQANKATL |
| | | YVDGVEVHNAKTKPREEQYNSTYRVVSVLTVLH | | VCLISDFYPGAVTVAWK |
| | | QDWLNGKEYKCKVSNKALGAPIEKTISKAKGQP | | ADSSPVKAGVETTTPSK |
| | | REPQVYTLPPSREEMTKNQVSLTCLVKGFYPSD | | QSNNKYAASSYLSLTPE |
| | | IAVEWESNGQPENNYKTTPPVLDSDGSFFLYSK | | QWKSHRSYSCQVTHEGS |
| | | LTVDKSRWQQGNVFSCSVMHEALHNHYTQKSLS | | TVEKTVAPTECS |
| | | LSPGK | | |
| A64- | 5679 | MGWSCIILFLVATATGVHSEVQLVESGGGLVQP | 5867 | MGWSCIILFLVATATGV |
| LALA | | GGSLRLSCAASGFTFSSYDMHWVRQATGKGLEW | | HSSSELTQDPAVSVALG |
| PGhL | | VSAIGTAGDTYYPGSVKGRFTISRENAKNSLYL | | QTVRITCQGDSLRSYYA |
| 2 | | QMNSLRAGDTAVYYCARYSGSYYYFDYWGQGTL | | SWYQQKPGQAPVLVIYG |
| | | VTVSSASTKGPSVFPLAPSSKSTSGGTAALGCL | | KNNRPSGIPDRFSGSSS |
| | | VKDYFPEPVTVSWNSGALTSGVHTFPAVLQSSG | | GNTASLTITGAQAEDEA |
| | | LYSLSSVVTVPSSSLGTQTYICNVNHKPSNTKV | | DYYCNSRDSSGNHVVFG |
| | | DKKVEPKSCDKTHTCPPCPAPEAAGGPSVFLFP | | GGTKLTVLGQPKAAPSV |
| | | PKPKDTLMISRTPEVTCVVVDVSHEDPEVKENW | | TLFPPSSEELQANKATL |
| | | YVDGVEVHNAKTKPREEQYNSTYRVVSVLTVLH | | VCLISDFYPGAVTVAWK |
| | | QDWLNGKEYKCKVSNKALGAPIEKTISKAKGQP | | ADSSPVKAGVETTTPSK |
| | | REPQVYTLPPSREEMTKNQVSLTCLVKGFYPSD | | QSNNKYAASSYLSLTPE |
| | | IAVEWESNGQPENNYKTTPPVLDSDGSFFLYSK | | QWKSHRSYSCQVTHEGS |
| | | LTVDKSRWQQGNVFSCSVMHEALHNHYTQKSLS | | TVEKTVAPTECS |
| | | LSPGK | | |
| A65- | 5680 | MGWSCIILFLVATATGVHSQVQLVESGGGVVQP | 5868 | MGWSCIILFLVATATGV |
| LALA | | GRSLRLSCAASGFTFSSYGMHWVRQAPGKGLEW | | HSSYELTQPPSVSVSPG |
| PGhL | | VAVIWYDGSNKYYADSVKGRFTISRDNSKNTLY | | QTARITCSGDALPKKYA |
| 2 | | LQMNSLRAEDTAVYYCARGIAAAGKDYWGQGTL | | YWYQQKSGQAPVLVIYE |
| | | VTVSSASTKGPSVFPLAPSSKSTSGGTAALGCL | | DSKRPSGIPERFSGSSS |
| | | VKDYFPEPVTVSWNSGALTSGVHTFPAVLQSSG | | GTMATLTISGAQVEDEA |
| | | LYSLSSVVTVPSSSLGTQTYICNVNHKPSNTKV | | DYYCYSTDSSGNHAVFG |
| | | DKKVEPKSCDKTHTCPPCPAPEAAGGPSVFLFP | | GGTQLTVLGQPKAAPSV |
| | | PKPKDTLMISRTPEVTCVVVDVSHEDPEVKENW | | TLFPPSSEELQANKATL |
| | | YVDGVEVHNAKTKPREEQYNSTYRVVSVLTVLH | | VCLISDFYPGAVTVAWK |
| | | QDWLNGKEYKCKVSNKALGAPIEKTISKAKGQP | | ADSSPVKAGVETTTPSK |
| | | REPQVYTLPPSREEMTKNQVSLTCLVKGFYPSD | | QSNNKYAASSYLSLTPE |
| | | IAVEWESNGQPENNYKTTPPVLDSDGSFFLYSK | | QWKSHRSYSCQVTHEGS |
| | | LTVDKSRWQQGNVFSCSVMHEALHNHYTQKSLS | | TVEKTVAPTECS |
| | | LSPGK | | |
| A66- | 5681 | MGWSCIILFLVATATGVHSEVQLVQSGAEVKKP | 5869 | MGWSCIILFLVATATGV |
| LALA | | GESLKISCKGSGYSFTSYWIGWVRQMPGKGLEW | | HSSYVLTQPPSVSVAPG |
| PGhL | | MGIIYPGDSDTRYSPSFQGQVTISADKSISTAY | | KTARITCGGNNIGSKSV |
| 2 | | LQWSSLKASDTAMYYCARQDSNYVFDYWGQGTL | | HWYQQKPGQAPVLVIYY |
| | | VTVSSASTKGPSVFPLAPSSKSTSGGTAALGCL | | DSDRPSGIPERFSGSNS |
| | | VKDYFPEPVTVSWNSGALTSGVHTFPAVLQSSG | | GNTATLTISRVEAGDEA |
| | | LYSLSSVVTVPSSSLGTQTYICNVNHKPSNTKV | | DYYCQVWDSSSDHVVFG |
| | | DKKVEPKSCDKTHTCPPCPAPEAAGGPSVFLFP | | GGTKLTVLGQPKAAPSV |
| | | PKPKDTLMISRTPEVTCVVVDVSHEDPEVKENW | | TLFPPSSEELQANKATL |
| | | YVDGVEVHNAKTKPREEQYNSTYRVVSVLTVLH | | VCLISDFYPGAVTVAWK |
| | | QDWLNGKEYKCKVSNKALGAPIEKTISKAKGQP | | ADSSPVKAGVETTTPSK |
| | | REPQVYTLPPSREEMTKNQVSLTCLVKGFYPSD | | QSNNKYAASSYLSLTPE |
| | | IAVEWESNGQPENNYKTTPPVLDSDGSFFLYSK | | QWKSHRSYSCQVTHEGS |
| | | LTVDKSRWQQGNVFSCSVMHEALHNHYTQKSLS | | TVEKTVAPTECS |
| | | LSPGK | | |
| A67- | 5682 | MGWSCIILFLVATATGVHSEVQLVESGGGLVQP | 5870 | MGWSCIILFLVATATGV |
| LALA | | GGSLRLSCAASGFTFSNYWMSWVRQAPGKGLEW | | HSQSVLTQPPSAFGTPG |
| PGhL | | VANIKYDGREQYYVDSVKGRFAISRDNAKNSLS | | QRVTISCSGSISNLGSN |
| 2 | | LQMNSLRAEDTAIYYCARDHSAWSFDYWGQGTL | | TVNWYQQLPGTAPKLLI |
| | | VTVSSASTKGPSVFPLAPSSKSTSGGTAALGCL | | YSNNQRPSGVPDRESGS |
| | | VKDYFPEPVTVSWNSGALTSGVHTFPAVLQSSG | | KSGTSASLAISGLQFED |
| | | LYSLSSVVTVPSSSLGTQTYICNVNHKPSNTKV | | EADYHCATWDDSLNGRV |
| | | DKKVEPKSCDKTHTCPPCPAPEAAGGPSVFLFP | | FGGGTKLTVLGQPKAAP |
| | | PKPKDTLMISRTPEVTCVVVDVSHEDPEVKENW | | SVTLFPPSSEELQANKA |
| | | YVDGVEVHNAKTKPREEQYNSTYRVVSVLTVLH | | TLVCLISDFYPGAVTVA |
| | | QDWLNGKEYKCKVSNKALGAPIEKTISKAKGQP | | WKADSSPVKAGVETTTP |
| | | REPQVYTLPPSREEMTKNQVSLTCLVKGFYPSD | | SKQSNNKYAASSYLSLT |
| | | IAVEWESNGQPENNYKTTPPVLDSDGSFFLYSK | | PEQWKSHRSYSCQVTHE |
| | | LTVDKSRWQQGNVFSCSVMHEALHNHYTQKSLS | | GSTVEKTVAPTECS |
| | | LSPGK | | |
| A68- | 5683 | MGWSCIILFLVATATGVHSEVQLVESGGGLVQP | 5871 | MGWSCIILFLVATATGV |
| LALA | | GGSLRLSCAASGFTFSSYWMSWVRQAPGKGLEW | | HSQSALTQPPSASGSPG |
| PGhL | | VANIKQDGSEKYYVDSVKGRFTISRDNAKNSLY | | QSVTISCTGTSSDVGGY |
| 2 | | LQMNSLRAEDTAVYYCARRRGSCSFDYWGQGTL | | NYVSWYQQHPGKAPKLM |
| | | VTVSSASTKGPSVFPLAPSSKSTSGGTAALGCL | | IYEVSKRPSGVPDRFSG |
| | | VKDYFPEPVTVSWNSGALTSGVHTFPAVLQSSG | | SKSGNTASLTVSGLQAE |
| | | LYSLSSVVTVPSSSLGTQTYICNVNHKPSNTKV | | DEADYYCSSYAGSNNLV |
| | | DKKVEPKSCDKTHTCPPCPAPEAAGGPSVFLFP | | FGGGTKLTVLGQPKAAP |
| | | PKPKDTLMISRTPEVTCVVVDVSHEDPEVKENW | | SVTLFPPSSEELQANKA |
| | | YVDGVEVHNAKTKPREEQYNSTYRVVSVLTVLH | | TLVCLISDFYPGAVTVA |
| | | QDWLNGKEYKCKVSNKALGAPIEKTISKAKGQP | | WKADSSPVKAGVETTTP |
| | | REPQVYTLPPSREEMTKNQVSLTCLVKGFYPSD | | SKQSNNKYAASSYLSLT |
| | | IAVEWESNGQPENNYKTTPPVLDSDGSFFLYSK | | PEQWKSHRSYSCQVTHE |
| | | LTVDKSRWQQGNVFSCSVMHEALHNHYTQKSLS | | GSTVEKTVAPTECS |
| | | LSPGK | | |
| A69- | 5684 | MGWSCIILFLVATATGVHSQVQLVQSGAEVKKP | 5872 | MGWSCIILFLVATATGV |
| LALA | | GASVKVSCKASGYTFTSYGISWVRQAPGQGLEW | | HSQSALTQPPSASGSPG |
| PGhL | | MGWISAYNGNTNYAQKLQGRVTMTTDTSTSTAY | | QSVTISCTGTSSDVGGY |
| 2 | | MELRSLRSDDTAVYYCARRSYANCFDYWGQGTL | | NYVSWYQQHPGKAPKLM |
| | | VTVSSASTKGPSVFPLAPSSKSTSGGTAALGCL | | IYEVSKRPSGVPDRFSG |
| | | VKDYFPEPVTVSWNSGALTSGVHTFPAVLQSSG | | SKSGNTASLTVSGLQAE |
| | | LYSLSSVVTVPSSSLGTQTYICNVNHKPSNTKV | | DEADYYCSSYAGSNNWV |
| | | DKKVEPKSCDKTHTCPPCPAPEAAGGPSVFLFP | | FGGGTKLTVLGQPKAAP |
| | | PKPKDTLMISRTPEVTCVVVDVSHEDPEVKENW | | SVTLFPPSSEELQANKA |
| | | YVDGVEVHNAKTKPREEQYNSTYRVVSVLTVLH | | TLVCLISDFYPGAVTVA |
| | | QDWLNGKEYKCKVSNKALGAPIEKTISKAKGQP | | WKADSSPVKAGVETTTP |
| | | REPQVYTLPPSREEMTKNQVSLTCLVKGFYPSD | | SKQSNNKYAASSYLSLT |
| | | IAVEWESNGQPENNYKTTPPVLDSDGSFFLYSK | | PEQWKSHRSYSCQVTHE |
| | | LTVDKSRWQQGNVFSCSVMHEALHNHYTQKSLS | | GSTVEKTVAPTECS |
| | | LSPGK | | |
| A70- | 5685 | MGWSCIILFLVATATGVHSEVQLVESGGGLVQP | 5873 | MGWSCIILFLVATATGV |
| LALA | | GGSLRLSCAASGFTFSSYWMHWVRQAPGKGLVW | | HSQSALTQPASVSGSPG |
| PGhL | | VSRINSDGSSTSYADSVKGRFTISRDNAKNTLY | | QSITISCTGTSSDVGGY |
| 2 | | LQMNSLRAEDTAVYYCARDEQLVPFDIWGQGTM | | NYVSWYQQHPGKAPKLM |
| | | VTVSSASTKGPSVFPLAPSSKSTSGGTAALGCL | | IYDVSKRPSGVSNRFSG |
| | | VKDYFPEPVTVSWNSGALTSGVHTFPAVLQSSG | | SKSGNTASLTISGLQAE |
| | | LYSLSSVVTVPSSSLGTQTYICNVNHKPSNTKV | | DEADYYCCSYAGSSTLV |
| | | DKKVEPKSCDKTHTCPPCPAPEAAGGPSVELFP | | FGGGTKLTVLGQPKAAP |
| | | PKPKDTLMISRTPEVTCVVVDVSHEDPEVKENW | | SVTLFPPSSEELQANKA |
| | | YVDGVEVHNAKTKPREEQYNSTYRVVSVLTVLH | | TLVCLISDFYPGAVTVA |
| | | QDWLNGKEYKCKVSNKALGAPIEKTISKAKGQP | | WKADSSPVKAGVETTTP |
| | | REPQVYTLPPSREEMTKNQVSLTCLVKGFYPSD | | SKQSNNKYAASSYLSLT |
| | | IAVEWESNGQPENNYKTTPPVLDSDGSFFLYSK | | PEQWKSHRSYSCQVTHE |
| | | LTVDKSRWQQGNVFSCSVMHEALHNHYTQKSLS | | GSTVEKTVAPTECS |
| | | LSPGK | | |
| A71- | 5686 | MGWSCIILFLVATATGVHSEVQLVESGGGLIQP | 5874 | MGWSCIILFLVATATGV |
| LALA | | GGSLRLSCAASGFTVSSNYMSWVRQAPGKGLEW | | HSQSALTQPASVSGSPG |
| PGhL | | VSVIYSGGSTYYADSVKGRFTISRDNSKNTLYL | | QSITISCTGTSSDVGGY |
| 2 | | QMNSLRAEDTAVYYCARDGAAAGDFQHWGQGTL | | NYVSWYQQHPGKAPKLM |
| | | VTVSSASTKGPSVFPLAPSSKSTSGGTAALGCL | | IYDVSKRPSGVSNRFSG |
| | | VKDYFPEPVTVSWNSGALTSGVHTFPAVLQSSG | | SKSGNTASLTISGLQAE |
| | | LYSLSSVVTVPSSSLGTQTYICNVNHKPSNTKV | | DEADYYCCSYAGSSTWV |
| | | DKKVEPKSCDKTHTCPPCPAPEAAGGPSVFLFP | | FGGGTKLTVLGQPKAAP |
| | | PKPKDTLMISRTPEVTCVVVDVSHEDPEVKENW | | SVTLFPPSSEELQANKA |
| | | YVDGVEVHNAKTKPREEQYNSTYRVVSVLTVLH | | TLVCLISDFYPGAVTVA |
| | | QDWLNGKEYKCKVSNKALGAPIEKTISKAKGQP | | WKADSSPVKAGVETTTP |
| | | REPQVYTLPPSREEMTKNQVSLTCLVKGFYPSD | | SKQSNNKYAASSYLSLT |
| | | IAVEWESNGQPENNYKTTPPVLDSDGSFFLYSK | | PEQWKSHRSYSCQVTHE |
| | | LTVDKSRWQQGNVFSCSVMHEALHNHYTQKSLS | | GSTVEKTVAPTECS |
| | | LSPGK | | |
| A72- | 5687 | MGWSCIILFLVATATGVHSEVQLVESGGGLVQP | 5875 | MGWSCIILFLVATATGV |
| LALA | | GRSLRLSCAASGFTEDDYAMHWVRQAPGKGLEW | | HSQSALTQPPSASGSPG |
| PGhL | | VSGISWNSGSIGYADSVKGRFTISRDNAKNSLY | | QSVTISCTGTSSDVGGY |
| 2 | | LQMNSLRAEDTALYYCAKDSGSYYFDYWGQGTL | | NYVSWYQQHPGKAPKLM |
| | | VTVSSASTKGPSVFPLAPSSKSTSGGTAALGCL | | IYEVSKRPSGVPDRFSG |
| | | VKDYFPEPVTVSWNSGALTSGVHTFPAVLQSSG | | SKSGNTASLTVSGLQAE |
| | | LYSLSSVVTVPSSSLGTQTYICNVNHKPSNTKV | | DEADYYCSSYAGSNNFV |
| | | DKKVEPKSCDKTHTCPPCPAPEAAGGPSVFLFP | | VFGGGTKLTVLGQPKAA |
| | | PKPKDTLMISRTPEVTCVVVDVSHEDPEVKENW | | PSVTLFPPSSEELQANK |
| | | YVDGVEVHNAKTKPREEQYNSTYRVVSVLTVLH | | ATLVCLISDFYPGAVTV |
| | | QDWLNGKEYKCKVSNKALGAPIEKTISKAKGQP | | AWKADSSPVKAGVETTT |
| | | REPQVYTLPPSREEMTKNQVSLTCLVKGFYPSD | | PSKQSNNKYAASSYLSL |
| | | IAVEWESNGQPENNYKTTPPVLDSDGSFFLYSK | | TPEQWKSHRSYSCQVTH |
| | | LTVDKSRWQQGNVFSCSVMHEALHNHYTQKSLS | | EGSTVEKTVAPTECS |
| | | LSPGK | | |
| A73- | 5688 | MGWSCIILFLVATATGVHSQVQLVESGGGLVKP | 5876 | MGWSCIILFLVATATGV |
| LALA | | GGSLRLSCAASGFTFSDYYMSWIRQAPGKGLEW | | HSQSVLTQPPSVSGAPG |
| PGhL | | VSYISSSGSTIYYADSVKGRFTISRDNAKNSLY | | QRVTISCTGSSSNIGAG |
| 2 | | LQMNSLRAEDTAVYYCARDGQLWSFDYWGQGTL | | YDVHWYQQLPGTAPKLL |
| | | VTVSSASTKGPSVFPLAPSSKSTSGGTAALGCL | | IYGNSNRPSGVPDRFSG |
| | | VKDYFPEPVTVSWNSGALTSGVHTFPAVLQSSG | | SKSGTSASLAITGLQAE |
| | | LYSLSSVVTVPSSSLGTQTYICNVNHKPSNTKV | | DEADYYCQSYDSSLSDV |
| | | DKKVEPKSCDKTHTCPPCPAPEAAGGPSVFLFP | | VFGGGTKLTVLGQPKAA |
| | | PKPKDTLMISRTPEVTCVVVDVSHEDPEVKENW | | PSVTLFPPSSEELQANK |
| | | YVDGVEVHNAKTKPREEQYNSTYRVVSVLTVLH | | ATLVCLISDFYPGAVTV |
| | | QDWLNGKEYKCKVSNKALGAPIEKTISKAKGQP | | AWKADSSPVKAGVETTT |
| | | REPQVYTLPPSREEMTKNQVSLTCLVKGFYPSD | | PSKQSNNKYAASSYLSL |
| | | IAVEWESNGQPENNYKTTPPVLDSDGSFFLYSK | | TPEQWKSHRSYSCQVTH |
| | | LTVDKSRWQQGNVFSCSVMHEALHNHYTQKSLS | | EGSTVEKTVAPTECS |
| | | LSPGK | | |
| A74- | 5689 | MGWSCIILFLVATATGVHSEVQLVESGGGLIQP | 5877 | MGWSCIILFLVATATGV |
| LALA | | GGSLRLSCAASGFTVSRNYMSWVRQAPGKGLEW | | HSQSVLTQPPSVSGAPG |
| PGhL | | VSIIYAGGNTYYADSVKGRFTISRDNSKNTLYL | | QRVTISCTGSSSNIGAG |
| 2 | | QMNSLRAEDTGVYYCGRVVPIGNWEDPWGQGTL | | YDVHWYQQLPGTAPKLL |
| | | VTVSSASTKGPSVFPLAPSSKSTSGGTAALGCL | | IYGNNNRPSGVPDRESG |
| | | VKDYFPEPVTVSWNSGALTSGVHTFPAVLQSSG | | SKSGTSASLAITGLQAE |
| | | LYSLSSVVTVPSSSLGTQTYICNVNHKPSNTKV | | DEADYYCQSYDSSLSGW |
| | | DKKVEPKSCDKTHTCPPCPAPEAAGGPSVFLFP | | VFGGGTKLTVLGQPKAA |
| | | PKPKDTLMISRTPEVTCVVVDVSHEDPEVKENW | | PSVTLFPPSSEELQANK |
| | | YVDGVEVHNAKTKPREEQYNSTYRVVSVLTVLH | | ATLVCLISDFYPGAVTV |
| | | QDWLNGKEYKCKVSNKALGAPIEKTISKAKGQP | | AWKADSSPVKAGVETTT |
| | | REPQVYTLPPSREEMTKNQVSLTCLVKGFYPSD | | PSKQSNNKYAASSYLSL |
| | | IAVEWESNGQPENNYKTTPPVLDSDGSFFLYSK | | TPEQWKSHRSYSCQVTH |
| | | LTVDKSRWQQGNVFSCSVMHEALHNHYTQKSLS | | EGSTVEKTVAPTECS |
| | | LSPGK | | |
| A75- | 5690 | MGWSCIILFLVATATGVHSEVQLVESGGGLVQP | 5878 | MGWSCIILFLVATATGV |
| LALA | | GGSLRLSCAASGFTFSSYWMSWVRQAPGKGLEW | | HSQAVLTQPASLSASPG |
| PGhL | | VANIKQDGSEKYYVDSVKGRFTISRDNAKNSLY | | ASASLTCTLRSGINVGT |
| 2 | | LQMNSLRAEDTAVYYCARDSNWGVFDYWGQGTL | | YRIYWYQQKPGSPPQYL |
| | | VTVSSASTKGPSVFPLAPSSKSTSGGTAALGCL | | LRYKSDSDKQQGSGVPS |
| | | VKDYFPEPVTVSWNSGALTSGVHTFPAVLQSSG | | RFSGSKDASANAGILLI |
| | | LYSLSSVVTVPSSSLGTQTYICNVNHKPSNTKV | | SGLQSEDEADYYCMIWH |
| | | DKKVEPKSCDKTHTCPPCPAPEAAGGPSVFLFP | | SSAWVFGGGTKLTVLGQ |
| | | PKPKDTLMISRTPEVTCVVVDVSHEDPEVKENW | | PKAAPSVTLFPPSSEEL |
| | | YVDGVEVHNAKTKPREEQYNSTYRVVSVLTVLH | | QANKATLVCLISDFYPG |
| | | QDWLNGKEYKCKVSNKALGAPIEKTISKAKGQP | | AVTVAWKADSSPVKAGV |
| | | REPQVYTLPPSREEMTKNQVSLTCLVKGFYPSD | | ETTTPSKQSNNKYAASS |
| | | IAVEWESNGQPENNYKTTPPVLDSDGSFFLYSK | | YLSLTPEQWKSHRSYSC |
| | | LTVDKSRWQQGNVFSCSVMHEALHNHYTQKSLS | | QVTHEGSTVEKTVAPTE |
| | | LSPGK | | CS |
| A76- | 5691 | MGWSCIILFLVATATGVHSEVQLVESGGGLVQP | 5879 | MGWSCIILFLVATATGV |
| LALA | | GGSLRLSCAASGFTFSSYWMSWVRQAPGKGLEW | | HSQAVLTQPASLSASPG |
| PGhL | | VANIKQDGSEKYYVDSVKGRFTISRDNAKNSLY | | ASASLTCTLRSGINVGT |
| 2 | | LQMNSLRAEDTAVYYCARDRLTGDLDYWGQGTL | | YRIYWYQQKPGSPPQYL |
| | | VTVSSASTKGPSVFPLAPSSKSTSGGTAALGCL | | LRYKSDSDKQQGSGVPS |
| | | VKDYFPEPVTVSWNSGALTSGVHTFPAVLQSSG | | RFSGSKDASANAGILLI |
| | | LYSLSSVVTVPSSSLGTQTYICNVNHKPSNTKV | | SGLQSEDEADYYCMIWH |
| | | DKKVEPKSCDKTHTCPPCPAPEAAGGPSVFLFP | | SSAWVFGGGTKLTVLGQ |
| | | PKPKDTLMISRTPEVTCVVVDVSHEDPEVKENW | | PKAAPSVTLFPPSSEEL |
| | | YVDGVEVHNAKTKPREEQYNSTYRVVSVLTVLH | | QANKATLVCLISDFYPG |
| | | QDWLNGKEYKCKVSNKALGAPIEKTISKAKGQP | | AVTVAWKADSSPVKAGV |
| | | REPQVYTLPPSREEMTKNQVSLTCLVKGFYPSD | | ETTTPSKQSNNKYAASS |
| | | IAVEWESNGQPENNYKTTPPVLDSDGSFFLYSK | | YLSLTPEQWKSHRSYSC |
| | | LTVDKSRWQQGNVFSCSVMHEALHNHYTQKSLS | | QVTHEGSTVEKTVAPTE |
| | | LSPGK | | CS |
| A77- | 5692 | MGWSCIILFLVATATGVHSEVQLVESGGGLVQP | 5880 | MGWSCIILFLVATATGV |
| LALA | | GGSLRLSCEASGFTFSRYWMHWVRQVPGKGLVW | | HSSYELTQPLSVSVALG |
| PGhL | | VSRINIVGSTIDYADSVKGRFTISRDNAKNTLY | | QSAMITCRGNNIGSQNV |
| 2 | | LQMDSLTAEDTAVYYCAREGDRSDAFAIWGQGT | | HWYHQKPGQAPVLVIYR |
| | | MVTVSSASTKGPSVFPLAPSSKSTSGGTAALGC | | NINRPSGIPERFSGSTS |
| | | LVKDYFPEPVTVSWNSGALTSGVHTFPAVLQSS | | GTTATLTISRAQAGDEA |
| | | GLYSLSSVVTVPSSSLGTQTYICNVNHKPSNTK | | DYYCQVWDSSGSWVFGG |
| | | VDKKVEPKSCDKTHTCPPCPAPEAAGGPSVFLF | | GTKLTVLGQPKAAPSVT |
| | | PPKPKDTLMISRTPEVTCVVVDVSHEDPEVKEN | | LFPPSSEELQANKATLV |
| | | WYVDGVEVHNAKTKPREEQYNSTYRVVSVLTVL | | CLISDFYPGAVTVAWKA |
| | | HQDWLNGKEYKCKVSNKALGAPIEKTISKAKGQ | | DSSPVKAGVETTTPSKQ |
| | | PREPQVYTLPPSREEMTKNQVSLTCLVKGFYPS | | SNNKYAASSYLSLTPEQ |
| | | DIAVEWESNGQPENNYKTTPPVLDSDGSFFLYS | | WKSHRSYSCQVTHEGST |
| | | KLTVDKSRWQQGNVFSCSVMHEALHNHYTQKSL | | VEKTVAPTECS |
| | | SLSPGK | | |
| A78- | 5693 | MGWSCIILFLVATATGVHSEVQLVESGGGLVKP | 5881 | MGWSCIILFLVATATGV |
| LALA | | GGSLRLSCAASGFTFSSYSMNWVRQAPGKGLEW | | HSSYELTQPPSVSVSPG |
| PGhL | | VSSISSSSSYIYYADSVKGRFTISRDNAKNSLY | | QTARITCSGDALPKKYA |
| 2 | | LQMNSLRAEDTAVYYCARQQWLGYYFDYWGQGT | | YWYQQKSGQAPVLVIYE |
| | | LVTVSSASTKGPSVFPLAPSSKSTSGGTAALGC | | DSKRPSGIPERFSGSSS |
| | | LVKDYFPEPVTVSWNSGALTSGVHTFPAVLQSS | | GTMATLTISGAQVEDEA |
| | | GLYSLSSVVTVPSSSLGTQTYICNVNHKPSNTK | | DYYCYSTDSSGNHRVFG |
| | | VDKKVEPKSCDKTHTCPPCPAPEAAGGPSVELF | | GGTKLTVLGQPKAAPSV |
| | | PPKPKDTLMISRTPEVTCVVVDVSHEDPEVKEN | | TLFPPSSEELQANKATL |
| | | WYVDGVEVHNAKTKPREEQYNSTYRVVSVLTVL | | VCLISDFYPGAVTVAWK |
| | | HQDWLNGKEYKCKVSNKALGAPIEKTISKAKGQ | | ADSSPVKAGVETTTPSK |
| | | PREPQVYTLPPSREEMTKNQVSLTCLVKGFYPS | | QSNNKYAASSYLSLTPE |
| | | DIAVEWESNGQPENNYKTTPPVLDSDGSFFLYS | | QWKSHRSYSCQVTHEGS |
| | | KLTVDKSRWQQGNVFSCSVMHEALHNHYTQKSL | | TVEKTVAPTECS |
| | | SLSPGK | | |
| A79- | 5694 | MGWSCIILFLVATATGVHSEVQLVESGGGLVQP | 5882 | MGWSCIILFLVATATGV |
| LALA | | GGSLRLSCAASGFTFSIYWMSWVRQAPGKGLEW | | HSSSELTQDPALSVALG |
| PGhL | | VATIKEDGSEKYYVDSVKGRFTISRDNAKNSLF | | QTVRITCQGDSLRSFYA |
| 2 | | LQMNSLRADDTAVYYCARDSNFLYYFDYWGQGT | | SWYQQKPGQAPVLVIYG |
| | | LVTVSSASTKGPSVFPLAPSSKSTSGGTAALGC | | KSNRPSGIPDRFSGSGS |
| | | LVKDYFPEPVTVSWNSGALTSGVHTFPAVLQSS | | GNTASLTITGAQAEDEA |
| | | GLYSLSSVVTVPSSSLGTQTYICNVNHKPSNTK | | DFYCNSRDTSGNYLVFG |
| | | VDKKVEPKSCDKTHTCPPCPAPEAAGGPSVFLF | | GGTKLTVLGQPKAAPSV |
| | | PPKPKDTLMISRTPEVTCVVVDVSHEDPEVKEN | | TLFPPSSEELQANKATL |
| | | WYVDGVEVHNAKTKPREEQYNSTYRVVSVLTVL | | VCLISDFYPGAVTVAWK |
| | | HQDWLNGKEYKCKVSNKALGAPIEKTISKAKGQ | | ADSSPVKAGVETTTPSK |
| | | PREPQVYTLPPSREEMTKNQVSLTCLVKGFYPS | | QSNNKYAASSYLSLTPE |
| | | DIAVEWESNGQPENNYKTTPPVLDSDGSFFLYS | | QWKSHRSYSCQVTHEGS |
| | | KLTVDKSRWQQGNVFSCSVMHEALHNHYTQKSL | | TVEKTVAPTECS |
| | | SLSPGK | | |
| A80- | 5695 | MGWSCIILFLVATATGVHSQVQLVQSGAEVKKP | 5883 | MGWSCIILFLVATATGV |
| LALA | | GASVKVSCKASGYTFTSYGISWVRQAPGQGLEW | | HSSSELTQDPAVSVALG |
| PGhL | | MGWISAYNGNTNYAQKLQGRVTMTTDTSTNTAY | | QTVRITCQGDSLRSYYA |
| 2 | | MELRSLRSDDTAVYYCARQITGTRGFDYWGQGT | | SWYQQKPGQAPVLVIYG |
| | | LVTVSSASTKGPSVFPLAPSSKSTSGGTAALGC | | KNNRPSGIPDRFSGSSS |
| | | LVKDYFPEPVTVSWNSGALTSGVHTFPAVLQSS | | GNTASLTITGAQAEDEA |
| | | GLYSLSSVVTVPSSSLGTQTYICNVNHKPSNTK | | DYYCNSRDSSGNHWVFG |
| | | VDKKVEPKSCDKTHTCPPCPAPEAAGGPSVELF | | GGTKLTVLGQPKAAPSV |
| | | PPKPKDTLMISRTPEVTCVVVDVSHEDPEVKEN | | TLFPPSSEELQANKATL |
| | | WYVDGVEVHNAKTKPREEQYNSTYRVVSVLTVL | | VCLISDFYPGAVTVAWK |
| | | HQDWLNGKEYKCKVSNKALGAPIEKTISKAKGQ | | ADSSPVKAGVETTTPSK |
| | | PREPQVYTLPPSREEMTKNQVSLTCLVKGFYPS | | QSNNKYAASSYLSLTPE |
| | | DIAVEWESNGQPENNYKTTPPVLDSDGSFFLYS | | QWKSHRSYSCQVTHEGS |
| | | KLTVDKSRWQQGNVFSCSVMHEALHNHYTQKSL | | TVEKTVAPTECS |
| | | SLSPGK | | |
| A81- | 5696 | MGWSCIILFLVATATGVHSQVQLVQSGAEVKKP | 5884 | MGWSCIILFLVATATGV |
| LALA | | GASVKVSCKASGYTFTSYDINWVRQATGQGLEW | | HSSYELTQPPSVSVSPG |
| PGhL | | MGWMNPNSGNTGYAQKFQGRVTMTRNTSISTAY | | QTARITCSGDALPKKYA |
| 2 | | MELSSLRSEDTAVYYCARMGYSNYPFDYWGQGT | | YWYQQKSGQAPVLVIYE |
| | | LVTVSSASTKGPSVFPLAPSSKSTSGGTAALGC | | DSKRPSGIPERFSGSSS |
| | | LVKDYFPEPVTVSWNSGALTSGVHTFPAVLQSS | | GTMATLTISGAQVEDEA |
| | | GLYSLSSVVTVPSSSLGTQTYICNVNHKPSNTK | | DYYCYSTDSSGNHVVFG |
| | | VDKKVEPKSCDKTHTCPPCPAPEAAGGPSVFLF | | GGTKLTVLGQPKAAPSV |
| | | PPKPKDTLMISRTPEVTCVVVDVSHEDPEVKEN | | TLFPPSSEELQANKATL |
| | | WYVDGVEVHNAKTKPREEQYNSTYRVVSVLTVL | | VCLISDFYPGAVTVAWK |
| | | HQDWLNGKEYKCKVSNKALGAPIEKTISKAKGQ | | ADSSPVKAGVETTTPSK |
| | | PREPQVYTLPPSREEMTKNQVSLTCLVKGFYPS | | QSNNKYAASSYLSLTPE |
| | | DIAVEWESNGQPENNYKTTPPVLDSDGSFFLYS | | QWKSHRSYSCQVTHEGS |
| | | KLTVDKSRWQQGNVFSCSVMHEALHNHYTQKSL | | TVEKTVAPTECS |
| | | SLSPGK | | |
| A82- | 5697 | MGWSCIILFLVATATGVHSQVQLVQSGSEVKKP | 5885 | MGWSCIILFLVATATGV |
| LALA | | GASVKVSCKASGYTFTSYYMHWVRQAPGQGLEW | | HSSSELTQDPAVSVALG |
| PGhL | | MGIINPSGGSTSYAQKFQGRVTMTRDTSTSTVY | | QTVRITCQGDSLRSYYA |
| 2 | | MELSSLRSEDTAVYYCARGIPTTVTPDYWGQGT | | SWYQQKPGQAPVLVIYG |
| | | LVTVSSASTKGPSVFPLAPSSKSTSGGTAALGC | | KNNRPSGIPDRFSGSSS |
| | | LVKDYFPEPVTVSWNSGALTSGVHTFPAVLQSS | | GNTASLTITGAQAEDEA |
| | | GLYSLSSVVTVPSSSLGTQTYICNVNHKPSNTK | | DYYCNSRDSSGNHLVFG |
| | | VDKKVEPKSCDKTHTCPPCPAPEAAGGPSVFLF | | GGTKLTVLGQPKAAPSV |
| | | PPKPKDTLMISRTPEVTCVVVDVSHEDPEVKEN | | TLFPPSSEELQANKATL |
| | | WYVDGVEVHNAKTKPREEQYNSTYRVVSVLTVL | | VCLISDFYPGAVTVAWK |
| | | HQDWLNGKEYKCKVSNKALGAPIEKTISKAKGQ | | ADSSPVKAGVETTTPSK |
| | | PREPQVYTLPPSREEMTKNQVSLTCLVKGFYPS | | QSNNKYAASSYLSLTPE |
| | | DIAVEWESNGQPENNYKTTPPVLDSDGSFFLYS | | QWKSHRSYSCQVTHEGS |
| | | KLTVDKSRWQQGNVFSCSVMHEALHNHYTQKSL | | TVEKTVAPTECS |
| | | SLSPGK | | |
| A83- | 5698 | MGWSCIILFLVATATGVHSEVQLVESGGGLVQP | 5886 | MGWSCIILFLVATATGV |
| LALA | | GGSLRLSCAASGFTFSSYDMHWVRQATGKGLEW | | HSSYELTQPPSVSVSPG |
| PGhL | | VSAIGTAGDTYYPGSVKGRFTISRENAKNSLYL | | QTARITCSGDALPKKYA |
| 2 | | QMNSLRAGDTAVYYCARAGLLTGDAFDIWGQGT | | YWYQQKSGQAPVLVIYE |
| | | MVTVSSASTKGPSVFPLAPSSKSTSGGTAALGC | | DSKRPSGIPERFSGSSS |
| | | LVKDYFPEPVTVSWNSGALTSGVHTFPAVLQSS | | GTMATLTISGAQVEDEA |
| | | GLYSLSSVVTVPSSSLGTQTYICNVNHKPSNTK | | DYYCYSTDSSGNHRVFG |
| | | VDKKVEPKSCDKTHTCPPCPAPEAAGGPSVFLF | | GGTKLTVLGQPKAAPSV |
| | | PPKPKDTLMISRTPEVTCVVVDVSHEDPEVKEN | | TLFPPSSEELQANKATL |
| | | WYVDGVEVHNAKTKPREEQYNSTYRVVSVLTVL | | VCLISDFYPGAVTVAWK |
| | | HQDWLNGKEYKCKVSNKALGAPIEKTISKAKGQ | | ADSSPVKAGVETTTPSK |
| | | PREPQVYTLPPSREEMTKNQVSLTCLVKGFYPS | | QSNNKYAASSYLSLTPE |
| | | DIAVEWESNGQPENNYKTTPPVLDSDGSFFLYS | | QWKSHRSYSCQVTHEGS |
| | | KLTVDKSRWQQGNVFSCSVMHEALHNHYTQKSL | | TVEKTVAPTECS |
| | | SLSPGK | | |
| A84- | 5699 | MGWSCIILFLVATATGVHSEVQLVESGGGLVKP | 5887 | MGWSCIILFLVATATGV |
| LALA | | GGSLRLSCAASGFTFSNAWMSWVRQAPGKGLEW | | HSQTVVTQEPSLTVSPG |
| PGhL | | VGRIKSKTDGGTTDYAAPVKGRFTISRDDSKNT | | GTVTLTCASSTGAVTSG |
| 2 | | LYLQMNSLKTEDTAVYYCITGTTFPFDYWGQGT | | YYPNWFQQKPGQTPRAL |
| | | LVTVSSASTKGPSVFPLAPSSKSTSGGTAALGC | | IYSTSNKHSWTPARFSG |
| | | LVKDYFPEPVTVSWNSGALTSGVHTFPAVLQSS | | SLLGGKAALTLSGVQPE |
| | | GLYSLSSVVTVPSSSLGTQTYICNVNHKPSNTK | | DEAEYYCLLYYGGAWVF |
| | | VDKKVEPKSCDKTHTCPPCPAPEAAGGPSVFLF | | GGGTKLTVLGQPKAAPS |
| | | PPKPKDTLMISRTPEVTCVVVDVSHEDPEVKEN | | VTLFPPSSEELQANKAT |
| | | WYVDGVEVHNAKTKPREEQYNSTYRVVSVLTVL | | LVCLISDFYPGAVTVAW |
| | | HQDWLNGKEYKCKVSNKALGAPIEKTISKAKGQ | | KADSSPVKAGVETTTPS |
| | | PREPQVYTLPPSREEMTKNQVSLTCLVKGFYPS | | KQSNNKYAASSYLSLTP |
| | | DIAVEWESNGQPENNYKTTPPVLDSDGSFFLYS | | EQWKSHRSYSCQVTHEG |
| | | KLTVDKSRWQQGNVFSCSVMHEALHNHYTQKSL | | STVEKTVAPTECS |
| | | SLSPGK | | |
| A85- | 5700 | MGWSCIILFLVATATGVHSEVQLVESGEGLVQP | 5888 | MGWSCIILFLVATATGV |
| LALA | | GGSLRLSCAASGFTFSSHAMHWVRQAPGKGLEY | | HSQSALTQPASVSGSPG |
| PGhL | | VSAISSNGGNTYYADSVKGRFTISRDNSKNTLY | | QSITISCTGTSSDVGGY |
| 2 | | LQVGSLRPEDMAIYYCTKGGVGASFDYWGQGTL | | NYVSWYQHHPGKAPKLM |
| | | VTVSSASTKGPSVFPLAPSSKSTSGGTAALGCL | | IYEVSNRPSGVSNRFSG |
| | | VKDYFPEPVTVSWNSGALTSGVHTFPAVLQSSG | | SKSGNTASLTISGLQAE |
| | | LYSLSSVVTVPSSSLGTQTYICNVNHKPSNTKV | | DEADYYCSSYTSSSTWV |
| | | DKKVEPKSCDKTHTCPPCPAPEAAGGPSVFLFP | | FGGGTKLTVLGQPKAAP |
| | | PKPKDTLMISRTPEVTCVVVDVSHEDPEVKENW | | SVTLFPPSSEELQANKA |
| | | YVDGVEVHNAKTKPREEQYNSTYRVVSVLTVLH | | TLVCLISDFYPGAVTVA |
| | | QDWLNGKEYKCKVSNKALGAPIEKTISKAKGQP | | WKADSSPVKAGVETTTP |
| | | REPQVYTLPPSREEMTKNQVSLTCLVKGFYPSD | | SKQSNNKYAASSYLSLT |
| | | IAVEWESNGQPENNYKTTPPVLDSDGSFFLYSK | | PEQWKSHRSYSCQVTHE |
| | | LTVDKSRWQQGNVFSCSVMHEALHNHYTQKSLS | | GSTVEKTVAPTECS |
| | | LSPGK | | |
| A86- | 5701 | MGWSCIILFLVATATGVHSEVQLVESGGGLVKP | 5889 | MGWSCIILFLVATATGV |
| LALA | | GGSLRLSCAASGFTFSSYSMNWVRQAPGKGLEW | | HSQSVLTQPPSASGTPG |
| PGhL | | VSSISSSSSYIYYADSVKGRFTISRDNAKNSLY | | QRVTISCSGSSSNIGSN |
| 2 | | LQMNSLRAEDTAVYYCARGDYSNYYFDYWGQGT | | TVNWYQQLPGTAPKLLI |
| | | LVTVSSASTKGPSVFPLAPSSKSTSGGTAALGC | | YSNNQRPSGVPDRFSGS |
| | | LVKDYFPEPVTVSWNSGALTSGVHTFPAVLQSS | | KSGTSASLAISGLQSED |
| | | GLYSLSSVVTVPSSSLGTQTYICNVNHKPSNTK | | EADYYCAAWDDSLNGWV |
| | | VDKKVEPKSCDKTHTCPPCPAPEAAGGPSVFLF | | FGGGTKLTVLGQPKAAP |
| | | PPKPKDTLMISRTPEVTCVVVDVSHEDPEVKEN | | SVTLFPPSSEELQANKA |
| | | WYVDGVEVHNAKTKPREEQYNSTYRVVSVLTVL | | TLVCLISDFYPGAVTVA |
| | | HQDWLNGKEYKCKVSNKALGAPIEKTISKAKGQ | | WKADSSPVKAGVETTTP |
| | | PREPQVYTLPPSREEMTKNQVSLTCLVKGFYPS | | SKQSNNKYAASSYLSLT |
| | | DIAVEWESNGQPENNYKTTPPVLDSDGSFFLYS | | PEQWKSHRSYSCQVTHE |
| | | KLTVDKSRWQQGNVFSCSVMHEALHNHYTQKSL | | GSTVEKTVAPTECS |
| | | SLSPGK | | |
| A87- | 5702 | MGWSCIILFLVATATGVHSQVQLQQWGAGLLKP | 5890 | MGWSCIILFLVATATGV |
| LALA | | SETLSLTCAVYGGSFSGYYWSWIRQPPGKGLEW | | HSQSALTQPPSASGSPG |
| PGhL | | IGEINHSGSTNYNPSLKSRVTISVDTSKNQFSL | | QSVTISCTGTSSDVGGY |
| 2 | | KLSSVTAADTAVYYCARWEQPWGQGTLVTVSSA | | NYVSWYQQHPGKAPKLM |
| | | STKGPSVFPLAPSSKSTSGGTAALGCLVKDYFP | | IYEVSKRPSGVPDRFSG |
| | | EPVTVSWNSGALTSGVHTFPAVLQSSGLYSLSS | | SKSGNTASLTVSGLQAE |
| | | VVTVPSSSLGTQTYICNVNHKPSNTKVDKKVEP | | DEADYYCSSYAGSNNWV |
| | | KSCDKTHTCPPCPAPEAAGGPSVFLFPPKPKDT | | FGGGTKLTVLGQPKAAP |
| | | LMISRTPEVTCVVVDVSHEDPEVKFNWYVDGVE | | SVTLFPPSSEELQANKA |
| | | VHNAKTKPREEQYNSTYRVVSVLTVLHQDWING | | TLVCLISDFYPGAVTVA |
| | | KEYKCKVSNKALGAPIEKTISKAKGQPREPQVY | | WKADSSPVKAGVETTTP |
| | | TLPPSREEMTKNQVSLTCLVKGFYPSDIAVEWE | | SKQSNNKYAASSYLSLT |
| | | SNGQPENNYKTTPPVLDSDGSFFLYSKLTVDKS | | PEQWKSHRSYSCQVTHE |
| | | RWQQGNVFSCSVMHEALHNHYTQKSLSLSPGK | | GSTVEKTVAPTECS |
| A88- | 5703 | MGWSCIILFLVATATGVHSQVQLVQSGAEVKKP | 5891 | MGWSCIILFLVATATGV |
| LALA | | GASVKVSCKASGYTFTSYYMHWVRQAPGQGLEW | | HSQSALTQPASVSGSPG |
| PGhL | | MGIINPSGGSTSYAQKFQGRVTMTRDTSTSTVY | | QSITISCTGTSSDVGGY |
| 2 | | MELSSLRSEDTAVYYCARRTGTTHYFDYWGQGT | | NYVSWYQQHPGKAPKLM |
| | | LVTVSSASTKGPSVFPLAPSSKSTSGGTAALGC | | IYDVSKRPSGVSNRFSG |
| | | LVKDYFPEPVTVSWNSGALTSGVHTFPAVLQSS | | SKSGNTASLTISGLQAE |
| | | GLYSLSSVVTVPSSSLGTQTYICNVNHKPSNTK | | DEADYYCCSYAGSSTLV |
| | | VDKKVEPKSCDKTHTCPPCPAPEAAGGPSVFLF | | FGGGTKLTVLGQPKAAP |
| | | PPKPKDTLMISRTPEVTCVVVDVSHEDPEVKEN | | SVTLFPPSSEELQANKA |
| | | WYVDGVEVHNAKTKPREEQYNSTYRVVSVLTVL | | TLVCLISDFYPGAVTVA |
| | | HQDWLNGKEYKCKVSNKALGAPIEKTISKAKGQ | | WKADSSPVKAGVETTTP |
| | | PREPQVYTLPPSREEMTKNQVSLTCLVKGFYPS | | SKQSNNKYAASSYLSLT |
| | | DIAVEWESNGQPENNYKTTPPVLDSDGSFFLYS | | PEQWKSHRSYSCQVTHE |
| | | KLTVDKSRWQQGNVFSCSVMHEALHNHYTQKSL | | GSTVEKTVAPTECS |
| | | SLSPGK | | |
| A89- | 5704 | MGWSCIILFLVATATGVHSQVQLVESGGGLVKP | 5892 | MGWSCIILFLVATATGV |
| LALA | | GGSLRLSCAASGFTFSDYYMSWIRQAPGKGLEW | | HSQSALTQPASVSGSPG |
| PGhL | | VSYISSSGSTIYYADSVKGRFTISRDNAKNSLY | | QSITISCTGTSSDVGGY |
| 2 | | LQMNSLRAEDTAVYYCARGLWLGLYFDYWGQGT | | NYVSWYQQHPGKAPKLM |
| | | LVTVSSASTKGPSVFPLAPSSKSTSGGTAALGC | | IYDVSKRPSGVSNRFSG |
| | | LVKDYFPEPVTVSWNSGALTSGVHTFPAVLQSS | | SKSGNTASLTISGLQAE |
| | | GLYSLSSVVTVPSSSLGTQTYICNVNHKPSNTK | | DEADYYCCSYAGSSTWV |
| | | VDKKVEPKSCDKTHTCPPCPAPEAAGGPSVFLF | | FGGGTKLTVLGQPKAAP |
| | | PPKPKDTLMISRTPEVTCVVVDVSHEDPEVKEN | | SVTLFPPSSEELQANKA |
| | | WYVDGVEVHNAKTKPREEQYNSTYRVVSVLTVL | | TLVCLISDFYPGAVTVA |
| | | HQDWLNGKEYKCKVSNKALGAPIEKTISKAKGQ | | WKADSSPVKAGVETTTP |
| | | PREPQVYTLPPSREEMTKNQVSLTCLVKGFYPS | | SKQSNNKYAASSYLSLT |
| | | DIAVEWESNGQPENNYKTTPPVLDSDGSFFLYS | | PEQWKSHRSYSCQVTHE |
| | | KLTVDKSRWQQGNVFSCSVMHEALHNHYTQKSL | | GSTVEKTVAPTECS |
| | | SLSPGK | | |
| A90- | 5705 | MGWSCIILFLVATATGVHSEVQLVQSGAEVKKP | 5893 | MGWSCIILFLVATATGV |
| LALA | | GESLKISCKGSGYSFTSYWIGWVRQMPGKGLEW | | HSQSALTQPPSASGSPG |
| PGhL | | MGIIYPGDSDTRYSPSFQGQVTISADKSISTAY | | QSVTISCTGTSSDVGGY |
| 2 | | LQWSSLKASDTAMYYCARFLGSSYYFDYWGQGT | | NYVSWYQQHPGKAPKLM |
| | | LVTVSSASTKGPSVFPLAPSSKSTSGGTAALGC | | IYEVSKRPSGVPDRFSG |
| | | LVKDYFPEPVTVSWNSGALTSGVHTFPAVLQSS | | SKSGNTASLTVSGLQAE |
| | | GLYSLSSVVTVPSSSLGTQTYICNVNHKPSNTK | | DEADYYCSSYAGSNNFE |
| | | VDKKVEPKSCDKTHTCPPCPAPEAAGGPSVELF | | VFGGGTKLTVLGQPKAA |
| | | PPKPKDTLMISRTPEVTCVVVDVSHEDPEVKEN | | PSVTLFPPSSEELQANK |
| | | WYVDGVEVHNAKTKPREEQYNSTYRVVSVLTVL | | ATLVCLISDFYPGAVTV |
| | | HQDWLNGKEYKCKVSNKALGAPIEKTISKAKGQ | | AWKADSSPVKAGVETTT |
| | | PREPQVYTLPPSREEMTKNQVSLTCLVKGFYPS | | PSKQSNNKYAASSYLSL |
| | | DIAVEWESNGQPENNYKTTPPVLDSDGSFFLYS | | TPEQWKSHRSYSCQVTH |
| | | KLTVDKSRWQQGNVFSCSVMHEALHNHYTQKSL | | EGSTVEKTVAPTECS |
| | | SLSPGK | | |
| A91- | 5706 | MGWSCIILFLVATATGVHSEVQLVESGGGLVQP | 5894 | MGWSCIILFLVATATGV |
| LALA | | GGSLRLSCAASGFTFSSYSMNWVRQAPGKGLEW | | HSQAVLTQPASLSASPG |
| PGhL | | VSYISSSSSTIYYADSVKGRFTISRDNAKNSLY | | ASASLTCTLRSGINVGT |
| 2 | | LQMNSLRDEDTAVYYCARGGAAAGAFDIWGQGT | | YRIYWYQQKPGSPPQYL |
| | | MVTVSSASTKGPSVFPLAPSSKSTSGGTAALGC | | LRYKSDSDKQQGSGVPS |
| | | LVKDYFPEPVTVSWNSGALTSGVHTFPAVLQSS | | RFSGSKDASANAGILLI |
| | | GLYSLSSVVTVPSSSLGTQTYICNVNHKPSNTK | | SGLQSEDEADYYCMIWH |
| | | VDKKVEPKSCDKTHTCPPCPAPEAAGGPSVFLF | | SSAWVFGGGTKLTVLGQ |
| | | PPKPKDTLMISRTPEVTCVVVDVSHEDPEVKEN | | PKAAPSVTLFPPSSEEL |
| | | WYVDGVEVHNAKTKPREEQYNSTYRVVSVLTVL | | QANKATLVCLISDFYPG |
| | | HQDWLNGKEYKCKVSNKALGAPIEKTISKAKGQ | | AVTVAWKADSSPVKAGV |
| | | PREPQVYTLPPSREEMTKNQVSLTCLVKGFYPS | | ETTTPSKQSNNKYAASS |
| | | DIAVEWESNGQPENNYKTTPPVLDSDGSFFLYS | | YLSLTPEQWKSHRSYSC |
| | | KLTVDKSRWQQGNVFSCSVMHEALHNHYTQKSL | | QVTHEGSTVEKTVAPTE |
| | | SLSPGK | | CS |
| A92- | 5707 | MGWSCIILFLVATATGVHSQVQLQESGPGLVKP | 5895 | MGWSCIILFLVATATGV |
| LALA | | SQTLSLTCTVSGGSISSGGYYWSWIRQHPGKGL | | HSSYELTQPPSVSVSPG |
| PGhL | | EWIGYIFYSGSTYYNPSLKSRVTISVDTSKKQY | | QTASITCSGDKLGDKYA |
| 2 | | SLKLRSVTAADTAVYYCARAEWELLWEDPWGQG | | CWYQQKPGQSPVVVIYQ |
| | | TLVTVSSASTKGPSVFPLAPSSKSTSGGTAALG | | DSKRPSGIPERFSGSNS |
| | | CLVKDYFPEPVTVSWNSGALTSGVHTFPAVLQS | | GNTATLTISGTQAMDEA |
| | | SGLYSLSSVVTVPSSSLGTQTYICNVNHKPSNT | | DYYCQAWDSSTVVFGGG |
| | | KVDKKVEPKSCDKTHTCPPCPAPEAAGGPSVFL | | TKLTVLGQPKAAPSVTL |
| | | FPPKPKDTLMISRTPEVTCVVVDVSHEDPEVKF | | FPPSSEELQANKATLVC |
| | | NWYVDGVEVHNAKTKPREEQYNSTYRVVSVLTV | | LISDFYPGAVTVAWKAD |
| | | LHQDWLNGKEYKCKVSNKALGAPIEKTISKAKG | | SSPVKAGVETTTPSKQS |
| | | QPREPQVYTLPPSREEMTKNQVSLTCLVKGFYP | | NNKYAASSYLSLTPEQW |
| | | SDIAVEWESNGQPENNYKTTPPVLDSDGSFFLY | | KSHRSYSCQVTHEGSTV |
| | | SKLTVDKSRWQQGNVFSCSVMHEALHNHYTQKS | | EKTVAPTECS |
| | | LSLSPGK | | |
| A93- | 5708 | MGWSCIILFLVATATGVHSQITLKESGPTLVKP | 5896 | MGWSCIILFLVATATGV |
| LALA | | TQTLTLTCTFSGFSLSTSGVGVGWIRQPPGKAL | | HSSYELTQPPSVSVSPG |
| PGhL | | EWLALIYWNDDKRYSPSLKSRLTITKDTSKNQV | | QTARITCSADALPKQYA |
| 2 | | VLTMTNMDPVDTATYFCAHNYFYISGYFYWGQG | | YWYQQKPGQAPVLVIYK |
| | | TLVTVSSASTKGPSVFPLAPSSKSTSGGTAALG | | DSERPSGIPERFSGSSS |
| | | CLVKDYFPEPVTVSWNSGALTSGVHTFPAVLQS | | GTSVTLTISGVQAEDEA |
| | | SGLYSLSSVVTVPSSSLGTQTYICNVNHKPSNT | | DYYCQSANSGTWVFGGG |
| | | KVDKKVEPKSCDKTHTCPPCPAPEAAGGPSVEL | | TKLTVLGQPKAAPSVTL |
| | | FPPKPKDTLMISRTPEVTCVVVDVSHEDPEVKF | | FPPSSEELQANKATLVC |
| | | NWYVDGVEVHNAKTKPREEQYNSTYRVVSVLTV | | LISDFYPGAVTVAWKAD |
| | | LHQDWLNGKEYKCKVSNKALGAPIEKTISKAKG | | SSPVKAGVETTTPSKQS |
| | | QPREPQVYTLPPSREEMTKNQVSLTCLVKGFYP | | NNKYAASSYLSLTPEQW |
| | | SDIAVEWESNGQPENNYKTTPPVLDSDGSFFLY | | KSHRSYSCQVTHEGSTV |
| | | SKLTVDKSRWQQGNVFSCSVMHEALHNHYTQKS | | EKTVAPTECS |
| | | LSLSPGK | | |
| A94- | 5709 | MGWSCIILFLVATATGVHSQVQLVESGGGVVQP | 5897 | MGWSCIILFLVATATGV |
| LALA | | GRSLRLSCAASGFTFSSYGMHWVRQAPGKGLEW | | HSSYELTQPPSVSVSPG |
| PGhL | | VAVISYDGSNKYYADSVKGRFTISRDNSKNTLY | | QTASITCSGDKLGDKYA |
| 2 | | LQMNSLRAEDTAVYYCAKDPLRVVNYMDVWGKG | | CWYQQKPGQSPVLVIYQ |
| | | TTVTVSSASTKGPSVFPLAPSSKSTSGGTAALG | | DTKRPSGIPERFSGSNS |
| | | CLVKDYFPEPVTVSWNSGALTSGVHTFPAVLQS | | GNTATLTISGTQAMDEA |
| | | SGLYSLSSVVTVPSSSLGTQTYICNVNHKPSNT | | DYYCQAWDSSTVVFGGG |
| | | KVDKKVEPKSCDKTHTCPPCPAPEAAGGPSVFL | | TKLTVLGQPKAAPSVTL |
| | | FPPKPKDTLMISRTPEVTCVVVDVSHEDPEVKE | | FPPSSEELQANKATLVC |
| | | NWYVDGVEVHNAKTKPREEQYNSTYRVVSVLTV | | LISDFYPGAVTVAWKAD |
| | | LHQDWLNGKEYKCKVSNKALGAPIEKTISKAKG | | SSPVKAGVETTTPSKQS |
| | | QPREPQVYTLPPSREEMTKNQVSLTCLVKGFYP | | NNKYAASSYLSLTPEQW |
| | | SDIAVEWESNGQPENNYKTTPPVLDSDGSFFLY | | KSHRSYSCQVTHEGSTV |
| | | SKLTVDKSRWQQGNVFSCSVMHEALHNHYTQKS | | EKTVAPTECS |
| | | LSLSPGK | | |
| A95- | 5710 | MGWSCIILFLVATATGVHSQITLKESGPTLVKP | 5898 | MGWSCIILFLVATATGV |
| LALA | | TQTLTLTCTFSGFSLSTSGVGVGWIRQPPGKAL | | HSSYELTQPPSVSVSPG |
| PGhL | | EWLALIYWSDDKRYSPSLKNRLTITKDTSKNQV | | QTARITCSADALPNQYA |
| 2 | | VLTMTNMDPLATATYYCAQTGYNSWSFDYWGQG | | YWYQQKPGQAPVLVIYK |
| | | TLVTVSSASTKGPSVFPLAPSSKSTSGGTAALG | | DSERPSGIPERFSGSSS |
| | | CLVKDYFPEPVTVSWNSGALTSGVHTFPAVLQS | | GTTVTLTISGVQAEDEA |
| | | SGLYSLSSVVTVPSSSLGTQTYICNVNHKPSNT | | DYYCQSADSSGTWVFGG |
| | | KVDKKVEPKSCDKTHTCPPCPAPEAAGGPSVFL | | GTKLTVLGQPKAAPSVT |
| | | FPPKPKDTLMISRTPEVTCVVVDVSHEDPEVKF | | LFPPSSEELQANKATLV |
| | | NWYVDGVEVHNAKTKPREEQYNSTYRVVSVLTV | | CLISDFYPGAVTVAWKA |
| | | LHQDWLNGKEYKCKVSNKALGAPIEKTISKAKG | | DSSPVKAGVETTTPSKQ |
| | | QPREPQVYTLPPSREEMTKNQVSLTCLVKGFYP | | SNNKYAASSYLSLTPEQ |
| | | SDIAVEWESNGQPENNYKTTPPVLDSDGSFFLY | | WKSHRSYSCQVTHEGST |
| | | SKLTVDKSRWQQGNVFSCSVMHEALHNHYTQKS | | VEKTVAPTECS |
| | | LSLSPGK | | |
| A96- | 5711 | MGWSCIILFLVATATGVHSQVQLVQSGAEVKKP | 5899 | MGWSCIILFLVATATGV |
| LALA | | GASVKVSCKASGYPFTSYGINWVRQAPGQGLEW | | HSSSELTQDPAVSVALG |
| PGhL | | MGWISAYNSNTNYAEKFQGRVTMTTDTSTTTAY | | QTVRITCQGDSLRSYYA |
| 2 | | MDLRSLRSDDTAVYYCAREDAWNYGWFDPWGQG | | SWYQQKPGQAPVLVIYG |
| | | TLVTVSSASTKGPSVFPLAPSSKSTSGGTAALG | | KNNRPSGIPDRFSGSSS |
| | | CLVKDYFPEPVTVSWNSGALTSGVHTFPAVLQS | | GNTASLTITGAQAEDEA |
| | | SGLYSLSSVVTVPSSSLGTQTYICNVNHKPSNT | | DYYCNSRDSSGNHVVFG |
| | | KVDKKVEPKSCDKTHTCPPCPAPEAAGGPSVFL | | GGTKLTVLGQPKAAPSV |
| | | FPPKPKDTLMISRTPEVTCVVVDVSHEDPEVKE | | TLFPPSSEELQANKATL |
| | | NWYVDGVEVHNAKTKPREEQYNSTYRVVSVLTV | | VCLISDFYPGAVTVAWK |
| | | LHQDWLNGKEYKCKVSNKALGAPIEKTISKAKG | | ADSSPVKAGVETTTPSK |
| | | QPREPQVYTLPPSREEMTKNQVSLTCLVKGFYP | | QSNNKYAASSYLSLTPE |
| | | SDIAVEWESNGQPENNYKTTPPVLDSDGSFFLY | | QWKSHRSYSCQVTHEGS |
| | | SKLTVDKSRWQQGNVFSCSVMHEALHNHYTQKS | | TVEKTVAPTECS |
| | | LSLSPGK | | |
| A97- | 5712 | MGWSCIILFLVATATGVHSQVQLVQSGAEVKKP | 5900 | MGWSCIILFLVATATGV |
| LALA | | GASVKVSCKASGYSFTGYDISWVRQAPRQGLEW | | HSSYELTQPPSVSVSPG |
| PGhL | | MGWISAYNGNTNYAQKFQARVTMTTDTSTSTAY | | QTARITCSGDALPKKYA |
| 2 | | MELRSLRSDDTAVYYCAREILWLGGYFDYWGQG | | YWYQQKSGQAPVLVIYE |
| | | TLVTVSSASTKGPSVFPLAPSSKSTSGGTAALG | | DSKRPSGIPERFSGSSS |
| | | CLVKDYFPEPVTVSWNSGALTSGVHTFPAVLQS | | GTMATLTISGAQVEDEA |
| | | SGLYSLSSVVTVPSSSLGTQTYICNVNHKPSNT | | DYYCYSTDSSGNHRVFG |
| | | KVDKKVEPKSCDKTHTCPPCPAPEAAGGPSVEL | | GGTKLTVLGQPKAAPSV |
| | | FPPKPKDTLMISRTPEVTCVVVDVSHEDPEVKF | | TLFPPSSEELQANKATL |
| | | NWYVDGVEVHNAKTKPREEQYNSTYRVVSVLTV | | VCLISDFYPGAVTVAWK |
| | | LHQDWLNGKEYKCKVSNKALGAPIEKTISKAKG | | ADSSPVKAGVETTTPSK |
| | | QPREPQVYTLPPSREEMTKNQVSLTCLVKGFYP | | QSNNKYAASSYLSLTPE |
| | | SDIAVEWESNGQPENNYKTTPPVLDSDGSFFLY | | QWKSHRSYSCQVTHEGS |
| | | SKLTVDKSRWQQGNVFSCSVMHEALHNHYTQKS | | TVEKTVAPTECS |
| | | LSLSPGK | | |
| A98- | 5713 | MGWSCIILFLVATATGVHSQVQLVQSGSELKKP | 5901 | MGWSCIILFLVATATGV |
| LALA | | GASVKVSCKASGYTFSSYDMNWIRQAPGQGLEW | | HSSSELTQDPAVSVALG |
| PGhL | | MGWINTNTGNPTYAQGFTGRFVFSLDTSVSTAY | | QTVRITCQGDSLRSYYA |
| 2 | | LQISSLKAEDTAVYYCAREYSSGWYYFDYWGQG | | SWYQQKPGQAPVLVIYG |
| | | TLVTVSSASTKGPSVFPLAPSSKSTSGGTAALG | | KNNRPSGIPDRFSGSSS |
| | | CLVKDYFPEPVTVSWNSGALTSGVHTFPAVLQS | | GNTASLTITGAQAEDEA |
| | | SGLYSLSSVVTVPSSSLGTQTYICNVNHKPSNT | | DYYCNSRDSSGNHLVFG |
| | | KVDKKVEPKSCDKTHTCPPCPAPEAAGGPSVEL | | GGTKLTVLGQPKAAPSV |
| | | FPPKPKDTLMISRTPEVTCVVVDVSHEDPEVKF | | TLFPPSSEELQANKATL |
| | | NWYVDGVEVHNAKTKPREEQYNSTYRVVSVLTV | | VCLISDFYPGAVTVAWK |
| | | LHQDWLNGKEYKCKVSNKALGAPIEKTISKAKG | | ADSSPVKAGVETTTPSK |
| | | QPREPQVYTLPPSREEMTKNQVSLTCLVKGFYP | | QSNNKYAASSYLSLTPE |
| | | SDIAVEWESNGQPENNYKTTPPVLDSDGSFFLY | | QWKSHRSYSCQVTHEGS |
| | | SKLTVDKSRWQQGNVFSCSVMHEALHNHYTQKS | | TVEKTVAPTECS |
| | | LSLSPGK | | |
| A99- | 5714 | MGWSCIILFLVATATGVHSQVQLVQSGAEVKKP | 5902 | MGWSCIILFLVATATGV |
| LALA | | GASVKVSCKASGYTFTGYYMHWVRQAPGQGLEW | | HSSYELTQPPSVSVSPG |
| PGhL | | MGWINPNSGGTNYAQKFQGRVTMTRDTSISTAY | | QTARITCSGDALPKKYA |
| 2 | | MELSRLRSDDTAVYYCARERIAVAPPFDYWGQG | | YWYQQKSGQAPVLVIYE |
| | | TLVTVSSASTKGPSVFPLAPSSKSTSGGTAALG | | DSKRPSGIPERFSGSSS |
| | | CLVKDYFPEPVTVSWNSGALTSGVHTFPAVLQS | | GTMATLTISGAQVEDEA |
| | | SGLYSLSSVVTVPSSSLGTQTYICNVNHKPSNT | | DYYCYSTDSSGNHRVFG |
| | | KVDKKVEPKSCDKTHTCPPCPAPEAAGGPSVEL | | GGTKLTVLGQPKAAPSV |
| | | FPPKPKDTLMISRTPEVTCVVVDVSHEDPEVKE | | TLFPPSSEELQANKATL |
| | | NWYVDGVEVHNAKTKPREEQYNSTYRVVSVLTV | | VCLISDFYPGAVTVAWK |
| | | LHQDWLNGKEYKCKVSNKALGAPIEKTISKAKG | | ADSSPVKAGVETTTPSK |
| | | QPREPQVYTLPPSREEMTKNQVSLTCLVKGFYP | | QSNNKYAASSYLSLTPE |
| | | SDIAVEWESNGQPENNYKTTPPVLDSDGSFFLY | | QWKSHRSYSCQVTHEGS |
| | | SKLTVDKSRWQQGNVFSCSVMHEALHNHYTQKS | | TVEKTVAPTECS |
| | | LSLSPGK | | |
| A100- | 5715 | MGWSCIILFLVATATGVHSQVQLVQSGAEVKKP | 5903 | MGWSCIILFLVATATGV |
| LALA | | GASVKVSCKASGYTFTSYDINWVRQATGQGLEW | | HSSYELTQPPSVSVSPG |
| PGhL | | MGWMNPNSGNTGYAQKFQGRVTMTRNTSISTAY | | QTARITCSGDALPKKYA |
| 2 | | MELSSLRSEDTAVYYCARAGWELPEYFQHWGQG | | YWYQQKSGQAPVLVIYE |
| | | TLVTVSSASTKGPSVFPLAPSSKSTSGGTAALG | | DSKRPSGIPERFSGSSS |
| | | CLVKDYFPEPVTVSWNSGALTSGVHTFPAVLQS | | GTMATLTISGAQVEDEA |
| | | SGLYSLSSVVTVPSSSLGTQTYICNVNHKPSNT | | DYYCYSTDSSGNHRVFG |
| | | KVDKKVEPKSCDKTHTCPPCPAPEAAGGPSVEL | | GGTKLTVLGQPKAAPSV |
| | | FPPKPKDTLMISRTPEVTCVVVDVSHEDPEVKF | | TLFPPSSEELQANKATL |
| | | NWYVDGVEVHNAKTKPREEQYNSTYRVVSVLTV | | VCLISDFYPGAVTVAWK |
| | | LHQDWLNGKEYKCKVSNKALGAPIEKTISKAKG | | ADSSPVKAGVETTTPSK |
| | | QPREPQVYTLPPSREEMTKNQVSLTCLVKGFYP | | QSNNKYAASSYLSLTPE |
| | | SDIAVEWESNGQPENNYKTTPPVLDSDGSFFLY | | QWKSHRSYSCQVTHEGS |
| | | SKLTVDKSRWQQGNVFSCSVMHEALHNHYTQKS | | TVEKTVAPTECS |
| | | LSLSPGK | | |
| A101- | 5716 | MGWSCIILFLVATATGVHSQVQLVQSGAEVKKP | 5904 | MGWSCIILFLVATATGV |
| LALA | | GASVKVSCKASGYTFTSYDINWVRQATGQGLEW | | HSSYELTQPPSVSVSPG |
| PGhL | | MGWMNPNSGNTGYAQKFQGRVTMTRNTSISTAY | | QTARITCSGDALPKKYA |
| 2 | | MELSSLRSEDTAVYYCARGGDDYSNLFDYWGQG | | YWYQQKSGQAPVLVIYE |
| | | TLVTVSSASTKGPSVFPLAPSSKSTSGGTAALG | | DSKRPSGIPERFSGSSS |
| | | CLVKDYFPEPVTVSWNSGALTSGVHTFPAVLQS | | GTMATLTISGAQVEDEA |
| | | SGLYSLSSVVTVPSSSLGTQTYICNVNHKPSNT | | DYYCYSTDSSGNHRVFG |
| | | KVDKKVEPKSCDKTHTCPPCPAPEAAGGPSVEL | | GGTKLTVLGQPKAAPSV |
| | | FPPKPKDTLMISRTPEVTCVVVDVSHEDPEVKF | | TLFPPSSEELQANKATL |
| | | NWYVDGVEVHNAKTKPREEQYNSTYRVVSVLTV | | VCLISDFYPGAVTVAWK |
| | | LHQDWLNGKEYKCKVSNKALGAPIEKTISKAKG | | ADSSPVKAGVETTTPSK |
| | | QPREPQVYTLPPSREEMTKNQVSLTCLVKGFYP | | QSNNKYAASSYLSLTPE |
| | | SDIAVEWESNGQPENNYKTTPPVLDSDGSFFLY | | QWKSHRSYSCQVTHEGS |
| | | SKLTVDKSRWQQGNVFSCSVMHEALHNHYTQKS | | TVEKTVAPTECS |
| | | LSLSPGK | | |
| A102- | 5717 | MGWSCIILFLVATATGVHSQVQLVQSGAEVKKP | 5905 | MGWSCIILFLVATATGV |
| LALA | | GSSVKVPCKASGDTFSNFAINWVRQAPGQGLEW | | HSSYVLTQPPSVSVAPG |
| PGhL | | MGGIIPIFATANYAQNFQGRVTITADESTSAAY | | KTARITCGGNNIGSKSV |
| 2 | | MEVSSLRFEDTAVYYCARTPLGIGRSFDLWGQG | | HWYQQTPGQAPVLVIYY |
| | | TMVTVSSASTKGPSVFPLAPSSKSTSGGTAALG | | DSDRPSGIPDRFSGSNS |
| | | CLVKDYFPEPVTVSWNSGALTSGVHTEPAVLQS | | GNTATLTISRVEAGDEA |
| | | SGLYSLSSVVTVPSSSLGTQTYICNVNHKPSNT | | DYYCQVWDSNSDHWVFG |
| | | KVDKKVEPKSCDKTHTCPPCPAPEAAGGPSVFL | | GGTKLTVLGQPKAAPSV |
| | | FPPKPKDTLMISRTPEVTCVVVDVSHEDPEVKE | | TLFPPSSEELQANKATL |
| | | NWYVDGVEVHNAKTKPREEQYNSTYRVVSVLTV | | VCLISDFYPGAVTVAWK |
| | | LHQDWLNGKEYKCKVSNKALGAPIEKTISKAKG | | ADSSPVKAGVETTTPSK |
| | | QPREPQVYTLPPSREEMTKNQVSLTCLVKGFYP | | QSNNKYAASSYLSLTPE |
| | | SDIAVEWESNGQPENNYKTTPPVLDSDGSFFLY | | QWKSHRSYSCQVTHEGS |
| | | SKLTVDKSRWQQGNVFSCSVMHEALHNHYTQKS | | TVEKTVAPTECS |
| | | LSLSPGK | | |
| A103- | 5718 | MGWSCIILFLVATATGVHSEVQLVESGGGLVKP | 5906 | MGWSCIILFLVATATGV |
| LALA | | GGSLRLSCAASGFTFSNAWMSWVRQAPGKGLEW | | HSSYELTQPPSVSVSPG |
| PGhL | | VGRIKRKTDGGTTDFASPVKGRFTISRDDSNNT | | QTARITCSADALPKQYA |
| 2 | | LYLQMNSLKTEDTAVYYCTTASTVTTGDYWGQG | | YWYQQKPGQAPVLVIYK |
| | | TLVTVSSASTKGPSVFPLAPSSKSTSGGTAALG | | DSERPSGIPERFSGSSS |
| | | CLVKDYFPEPVTVSWNSGALTSGVHTFPAVLQS | | GTTVTLTISGVQAEDEA |
| | | SGLYSLSSVVTVPSSSLGTQTYICNVNHKPSNT | | DYYCQSADSSGTYPVFG |
| | | KVDKKVEPKSCDKTHTCPPCPAPEAAGGPSVEL | | GGTKLTVLGQPKAAPSV |
| | | FPPKPKDTLMISRTPEVTCVVVDVSHEDPEVKF | | TLFPPSSEELQANKATL |
| | | NWYVDGVEVHNAKTKPREEQYNSTYRVVSVLTV | | VCLISDFYPGAVTVAWK |
| | | LHQDWLNGKEYKCKVSNKALGAPIEKTISKAKG | | ADSSPVKAGVETTTPSK |
| | | QPREPQVYTLPPSREEMTKNQVSLTCLVKGFYP | | QSNNKYAASSYLSLTPE |
| | | SDIAVEWESNGQPENNYKTTPPVLDSDGSFFLY | | QWKSHRSYSCQVTHEGS |
| | | SKLTVDKSRWQQGNVFSCSVMHEALHNHYTQKS | | TVEKTVAPTECS |
| | | LSLSPGK | | |
| A104- | 5719 | MGWSCIILFLVATATGVHSQVQLQQSGPGLVKP | 5907 | MGWSCIILFLVATATGV |
| LALA | | SQTLSLTCAISGDSVSSNSAAWNWIRQSPSRGL | | HSSSELTQDPAVSVALG |
| PGhL | | EWLGRTYYRSKWYNDYAVSVKSRITINPDTSKN | | QTVRITCQGDSLRSYYA |
| 2 | | QFSLQLNSVTPEDTAVYYCARERTEIDYWGQGT | | SWYQQKPGQAPVLVIYG |
| | | LVTVSSASTKGPSVFPLAPSSKSTSGGTAALGC | | KNNRPSGIPDRFSGSSS |
| | | LVKDYFPEPVTVSWNSGALTSGVHTFPAVLQSS | | GNTASLTITGAQAEDEA |
| | | GLYSLSSVVTVPSSSLGTQTYICNVNHKPSNTK | | DYYCNSRDSSGNHWVFG |
| | | VDKKVEPKSCDKTHTCPPCPAPEAAGGPSVFLF | | GGTKLTVLGQPKAAPSV |
| | | PPKPKDTLMISRTPEVTCVVVDVSHEDPEVKEN | | TLFPPSSEELQANKATL |
| | | WYVDGVEVHNAKTKPREEQYNSTYRVVSVLTVL | | VCLISDFYPGAVTVAWK |
| | | HQDWLNGKEYKCKVSNKALGAPIEKTISKAKGQ | | ADSSPVKAGVETTTPSK |
| | | PREPQVYTLPPSREEMTKNQVSLTCLVKGFYPS | | QSNNKYAASSYLSLTPE |
| | | DIAVEWESNGQPENNYKTTPPVLDSDGSFFLYS | | QWKSHRSYSCQVTHEGS |
| | | KLTVDKSRWQQGNVFSCSVMHEALHNHYTQKSL | | TVEKTVAPTECS |
| | | SLSPGK | | |
| A105- | 5720 | MGWSCIILFLVATATGVHSEVQLVESGGGLVKP | 5908 | MGWSCIILFLVATATGV |
| LALA | | GGSLRLSCAASGFTFNNAWMIWVRQAPGKGLEW | | HSQTVVTQEPSLTVSPG |
| PGhL | | VGRIKSKTDGGTTDYGAPVKGRFTISRDDSKNT | | GTVTLTCGSSTGAVTSG |
| 2 | | LYLQMNSLKTEDTAVYYCTTGRYFDWFDYWGQG | | HYPYWFQQKPGQAPRTL |
| | | TLVTVSSASTKGPSVFPLAPSSKSTSGGTAALG | | IYDTSNKHSWTPARFSG |
| | | CLVKDYFPEPVTVSWNSGALTSGVHTFPAVLQS | | SLLGGKAALTLSGAQPE |
| | | SGLYSLSSVVTVPSSSLGTQTYICNVNHKPSNT | | DEAEYYCLLSYSGARVF |
| | | KVDKKVEPKSCDKTHTCPPCPAPEAAGGPSVEL | | GGGTKLTVLGQPKAAPS |
| | | FPPKPKDTLMISRTPEVTCVVVDVSHEDPEVKF | | VTLFPPSSEELQANKAT |
| | | NWYVDGVEVHNAKTKPREEQYNSTYRVVSVLTV | | LVCLISDFYPGAVTVAW |
| | | LHQDWLNGKEYKCKVSNKALGAPIEKTISKAKG | | KADSSPVKAGVETTTPS |
| | | QPREPQVYTLPPSREEMTKNQVSLTCLVKGFYP | | KQSNNKYAASSYLSLTP |
| | | SDIAVEWESNGQPENNYKTTPPVLDSDGSFFLY | | EQWKSHRSYSCQVTHEG |
| | | SKLTVDKSRWQQGNVFSCSVMHEALHNHYTQKS | | STVEKTVAPTECS |
| | | LSLSPGK | | |
| A106- | 5721 | MGWSCIILFLVATATGVHSEVQLVESGGGLVKP | 5909 | MGWSCIILFLVATATGV |
| LALA | | GGSLRLSCAASGFTFSNAWMSWVRQAPGKGLEW | | HSQSALTQPPSASGSPG |
| PGhL | | VGRIKSKTDGGTTDYAAPVKGRFTISRDDSKNT | | QSVTISCTGTSSDVGGY |
| 2 | | LYLQMNSLKTEDTAVYYCTTASGSYWEDPWGQG | | NYVSWYQQHPGKAPKLM |
| | | TLVTVSSASTKGPSVFPLAPSSKSTSGGTAALG | | IYEVSKRPSGVPDRFSG |
| | | CLVKDYFPEPVTVSWNSGALTSGVHTFPAVLQS | | SKSGNTASLTVSGLQAE |
| | | SGLYSLSSVVTVPSSSLGTQTYICNVNHKPSNT | | DEADYYCSSYAGSNNLV |
| | | KVDKKVEPKSCDKTHTCPPCPAPEAAGGPSVEL | | FGGGTKLTVLGQPKAAP |
| | | FPPKPKDTLMISRTPEVTCVVVDVSHEDPEVKF | | SVTLFPPSSEELQANKA |
| | | NWYVDGVEVHNAKTKPREEQYNSTYRVVSVLTV | | TLVCLISDFYPGAVTVA |
| | | LHQDWLNGKEYKCKVSNKALGAPIEKTISKAKG | | WKADSSPVKAGVETTTP |
| | | QPREPQVYTLPPSREEMTKNQVSLTCLVKGFYP | | SKQSNNKYAASSYLSLT |
| | | SDIAVEWESNGQPENNYKTTPPVLDSDGSFFLY | | PEQWKSHRSYSCQVTHE |
| | | SKLTVDKSRWQQGNVFSCSVMHEALHNHYTQKS | | GSTVEKTVAPTECS |
| | | LSLSPGK | | |
| A107- | 5722 | MGWSCIILFLVATATGVHSQVQLVESGGGVVQP | 5910 | MGWSCIILFLVATATGV |
| LALA | | GRSLRLSCAASGFTFSSYGMHWVRQAPGKGLEW | | HSQSALTQPASVSGSPG |
| PGhL | | VAVISYDGSNKYYADSVKGRFTISRDNSKNTLY | | QSITISCTGTSSDVGGY |
| 2 | | LQMNSLRAEDTAVYYCAKGNWNYGDAFDIWGQG | | NYVSWYQQHPGKAPKLM |
| | | TMVTVSSASTKGPSVFPLAPSSKSTSGGTAALG | | IYDVSKRPSGVSNRFSG |
| | | CLVKDYFPEPVTVSWNSGALTSGVHTFPAVLQS | | SKSGNTASLTISGLQAE |
| | | SGLYSLSSVVTVPSSSLGTQTYICNVNHKPSNT | | DEADYYCCSYAGSSTYV |
| | | KVDKKVEPKSCDKTHTCPPCPAPEAAGGPSVFL | | FGTGTKVTVLGQPKAAP |
| | | FPPKPKDTLMISRTPEVTCVVVDVSHEDPEVKF | | SVTLFPPSSEELQANKA |
| | | NWYVDGVEVHNAKTKPREEQYNSTYRVVSVLTV | | TLVCLISDFYPGAVTVA |
| | | LHQDWLNGKEYKCKVSNKALGAPIEKTISKAKG | | WKADSSPVKAGVETTTP |
| | | QPREPQVYTLPPSREEMTKNQVSLTCLVKGFYP | | SKQSNNKYAASSYLSLT |
| | | SDIAVEWESNGQPENNYKTTPPVLDSDGSFFLY | | PEQWKSHRSYSCQVTHE |
| | | SKLTVDKSRWQQGNVFSCSVMHEALHNHYTQKS | | GSTVEKTVAPTECS |
| | | LSLSPGK | | |
| A108- | 5723 | MGWSCIILFLVATATGVHSQVQLVESGGGVVRP | 5911 | MGWSCIILFLVATATGV |
| LALA | | GGSLRLSCAASGFTFDDYGMSWVRQAPGKGLEW | | HSQSALTQPASVSGSPG |
| PGhL | | VSGINWNGGSTGYADSVKGRFTISRDNAKNSLY | | QSITISCTGTSSDVGGY |
| 2 | | LQMNSLRAEDTALYYCARENYDFWSGFDPWGQG | | NYVSWYQQHPGKAPKLM |
| | | TLVTVSSASTKGPSVFPLAPSSKSTSGGTAALG | | IYEVSNRPSGVSNRFSG |
| | | CLVKDYFPEPVTVSWNSGALTSGVHTFPAVLQS | | SKSGNTASLTISGLQAE |
| | | SGLYSLSSVVTVPSSSLGTQTYICNVNHKPSNT | | DEADYYCSSYTSSSTVV |
| | | KVDKKVEPKSCDKTHTCPPCPAPEAAGGPSVEL | | FGGGTKLTVLGQPKAAP |
| | | FPPKPKDTLMISRTPEVTCVVVDVSHEDPEVKF | | SVTLFPPSSEELQANKA |
| | | NWYVDGVEVHNAKTKPREEQYNSTYRVVSVLTV | | TLVCLISDFYPGAVTVA |
| | | LHQDWLNGKEYKCKVSNKALGAPIEKTISKAKG | | WKADSSPVKAGVETTTP |
| | | QPREPQVYTLPPSREEMTKNQVSLTCLVKGFYP | | SKQSNNKYAASSYLSLT |
| | | SDIAVEWESNGQPENNYKTTPPVLDSDGSFFLY | | PEQWKSHRSYSCQVTHE |
| | | SKLTVDKSRWQQGNVFSCSVMHEALHNHYTQKS | | GSTVEKTVAPTECS |
| | | LSLSPGK | | |
| A109- | 5724 | MGWSCIILFLVATATGVHSQVQLQQSGPGLVKP | 5912 | MGWSCIILFLVATATGV |
| LALA | | SQTLSLTCAISGDSVSSNSAAWNWIRQSPSRGL | | HSQSALTQPASVSGSPG |
| PGhL | | EWLGRTYYRSKWYNGYAESVKSRITINPDTSKN | | QSITISCTGTSSDVGGY |
| 2 | | QFSLQLNSVTPEDTAVYYCAREDRGEDYWGQGT | | NYVSWYQQHPGKAPKLM |
| | | LVTVSSASTKGPSVFPLAPSSKSTSGGTAALGC | | IYDVSKRPSGVSNRFSG |
| | | LVKDYFPEPVTVSWNSGALTSGVHTFPAVLQSS | | SKSGNTASLTISGLQAE |
| | | GLYSLSSVVTVPSSSLGTQTYICNVNHKPSNTK | | DEADYYCCSYAGSSNVV |
| | | VDKKVEPKSCDKTHTCPPCPAPEAAGGPSVFLF | | FGGGTQLTVLGQPKAAP |
| | | PPKPKDTLMISRTPEVTCVVVDVSHEDPEVKEN | | SVTLFPPSSEELQANKA |
| | | WYVDGVEVHNAKTKPREEQYNSTYRVVSVLTVL | | TLVCLISDFYPGAVTVA |
| | | HQDWLNGKEYKCKVSNKALGAPIEKTISKAKGQ | | WKADSSPVKAGVETTTP |
| | | PREPQVYTLPPSREEMTKNQVSLTCLVKGFYPS | | SKQSNNKYAASSYLSLT |
| | | DIAVEWESNGQPENNYKTTPPVLDSDGSFFLYS | | PEQWKSHRSYSCQVTHE |
| | | KLTVDKSRWQQGNVFSCSVMHEALHNHYTQKSL | | GSTVEKTVAPTECS |
| | | SLSPGK | | |
| A110- | 5725 | MGWSCIILFLVATATGVHSEVQLVESGGGLVQP | 5913 | MGWSCIILFLVATATGV |
| LALA | | GRSLRLSCAASGFTFDDYAMHWVRQAPGKGLEW | | HSQSALTQPASVSGSPG |
| PGhL | | VSGISWNSGSIGYADSVKGRFTISRDNAKNSLY | | QSITISCTGTSSDVGGY |
| 2 | | LQMNSLRAEDTALYYCAKRAVVTDYYMDVWGKG | | NYVSWYQQHPGKAPKLM |
| | | TTVTVSSASTKGPSVFPLAPSSKSTSGGTAALG | | IYDVSKRPSGVSNRFSG |
| | | CLVKDYFPEPVTVSWNSGALTSGVHTFPAVLQS | | SKSGNTASLTISGLQAE |
| | | SGLYSLSSVVTVPSSSLGTQTYICNVNHKPSNT | | DEADYYCCSYAGSSTFW |
| | | KVDKKVEPKSCDKTHTCPPCPAPEAAGGPSVFL | | VFGGGTKLTVLGQPKAA |
| | | FPPKPKDTLMISRTPEVTCVVVDVSHEDPEVKF | | PSVTLFPPSSEELQANK |
| | | NWYVDGVEVHNAKTKPREEQYNSTYRVVSVLTV | | ATLVCLISDFYPGAVTV |
| | | LHQDWLNGKEYKCKVSNKALGAPIEKTISKAKG | | AWKADSSPVKAGVETTT |
| | | QPREPQVYTLPPSREEMTKNQVSLTCLVKGFYP | | PSKQSNNKYAASSYLSL |
| | | SDIAVEWESNGQPENNYKTTPPVLDSDGSFFLY | | TPEQWKSHRSYSCQVTH |
| | | SKLTVDKSRWQQGNVFSCSVMHEALHNHYTQKS | | EGSTVEKTVAPTECS |
| | | LSLSPGK | | |
| A111- | 5726 | MGWSCIILFLVATATGVHSEVQLVESGGGLVQP | 5914 | MGWSCIILFLVATATGV |
| LALA | | GGSLRLSCAASGFTFSSYSMNWVRQAPGKGLEW | | HSQSALTQPPSASGSPG |
| PGhL | | VSYISSSSNTIYYADSVKGRFTISRDNAKNSLY | | QSVTISCTGTSSDVGGY |
| 2 | | LQMSSLRDEDTAVYYCARTSSWSYDAFDIWGQG | | NYVSWYQQHPGKAPKLM |
| | | TMVTVSSASTKGPSVFPLAPSSKSTSGGTAALG | | IYEVSKRPSGVPDRFSG |
| | | CLVKDYFPEPVTVSWNSGALTSGVHTFPAVLQS | | SKSGNTASLTVSGLQAE |
| | | SGLYSLSSVVTVPSSSLGTQTYICNVNHKPSNT | | DEADYYCSSYAGSNNFV |
| | | KVDKKVEPKSCDKTHTCPPCPAPEAAGGPSVEL | | VFGGGTKLTVLGQPKAA |
| | | FPPKPKDTLMISRTPEVTCVVVDVSHEDPEVKF | | PSVTLFPPSSEELQANK |
| | | NWYVDGVEVHNAKTKPREEQYNSTYRVVSVLTV | | ATLVCLISDFYPGAVTV |
| | | LHQDWLNGKEYKCKVSNKALGAPIEKTISKAKG | | AWKADSSPVKAGVETTT |
| | | QPREPQVYTLPPSREEMTKNQVSLTCLVKGFYP | | PSKQSNNKYAASSYLSL |
| | | SDIAVEWESNGQPENNYKTTPPVLDSDGSFFLY | | TPEQWKSHRSYSCQVTH |
| | | SKLTVDKSRWQQGNVFSCSVMHEALHNHYTQKS | | EGSTVEKTVAPTECS |
| | | LSLSPGK | | |
| A112- | 5727 | MGWSCIILFLVATATGVHSQVQLVQSGAEVKKP | 5915 | MGWSCIILFLVATATGV |
| LALA | | GASVKVSCKASGYTFTSYYMHWVRQAPGQGLEW | | HSSYELTQPPSVSVSPG |
| PGhL | | MGIINPSGGSTSYAQKFQGRVTMTRDTSTSTVY | | QTASITCSGDKLGDKYA |
| 2 | | MELSSLRSEDTAVYYCARERGHTVTPYFDYWGQ | | CWYQQKPGQSPVLVIYQ |
| | | GTLVTVSSASTKGPSVFPLAPSSKSTSGGTAAL | | DSKRPSGIPERFSGSNS |
| | | GCLVKDYFPEPVTVSWNSGALTSGVHTFPAVLQ | | GNTATLTISGTQAMDEA |
| | | SSGLYSLSSVVTVPSSSLGTQTYICNVNHKPSN | | DYYCQATEVFGGGTKLT |
| | | TKVDKKVEPKSCDKTHTCPPCPAPEAAGGPSVF | | VLGQPKAAPSVTLFPPS |
| | | LFPPKPKDTLMISRTPEVTCVVVDVSHEDPEVK | | SEELQANKATLVCLISD |
| | | FNWYVDGVEVHNAKTKPREEQYNSTYRVVSVLT | | FYPGAVTVAWKADSSPV |
| | | VLHQDWLNGKEYKCKVSNKALGAPIEKTISKAK | | KAGVETTTPSKQSNNKY |
| | | GQPREPQVYTLPPSREEMTKNQVSLTCLVKGFY | | AASSYLSLTPEQWKSHR |
| | | PSDIAVEWESNGQPENNYKTTPPVLDSDGSFFL | | SYSCQVTHEGSTVEKTV |
| | | YSKLTVDKSRWQQGNVFSCSVMHEALHNHYTQK | | APTECS |
| | | SLSLSPGK | | |
| A113- | 5728 | MGWSCIILFLVATATGVHSEVQLVESGGGLVQP | 5916 | MGWSCIILFLVATATGV |
| LALA | | GGSLRLSCAASGFNFSSYNMNWVRQTPGKGLEW | | HSSYELTQPSSVSVSPG |
| PGhL | | VSYISNTGNTIYYVDSVKGRFTISRDNAKNSLY | | QTAKITCSGDVLAKKYA |
| 2 | | LQLNSLRDEDTAVYFCARDGPQVGATDFDYWGQ | | RWFQQKPGQVPVLVIYK |
| | | GTLVTVSSASTKGPSVFPLAPSSKSTSGGTAAL | | DSERPSGIPERFSGSSS |
| | | GCLVKDYFPEPVTVSWNSGALTSGVHTFPAVLQ | | GATVTLTISGAQVEDEA |
| | | SSGLYSLSSVVTVPSSSLGTQTYICNVNHKPSN | | DYYCYSAADNKVFGGGT |
| | | TKVDKKVEPKSCDKTHTCPPCPAPEAAGGPSVE | | KLTVLGQPKAAPSVTLF |
| | | LFPPKPKDTLMISRTPEVTCVVVDVSHEDPEVK | | PPSSEELQANKATLVCL |
| | | FNWYVDGVEVHNAKTKPREEQYNSTYRVVSVLT | | ISDFYPGAVTVAWKADS |
| | | VLHQDWLNGKEYKCKVSNKALGAPIEKTISKAK | | SPVKAGVETTTPSKQSN |
| | | GQPREPQVYTLPPSREEMTKNQVSLTCLVKGFY | | NKYAASSYLSLTPEQWK |
| | | PSDIAVEWESNGQPENNYKTTPPVLDSDGSFFL | | SHRSYSCQVTHEGSTVE |
| | | YSKLTVDKSRWQQGNVFSCSVMHEALHNHYTQK | | KTVAPTECS |
| | | SLSLSPGK | | |
| A114- | 5729 | MGWSCIILFLVATATGVHSEVQLVESGGGLVKP | 5917 | MGWSCIILFLVATATGV |
| LALA | | GGSLRLSCAASGFTFSNAWMSWVRQAPGKGLEW | | HSSYELTQPPSVSVSPG |
| PGhL | | VGRIKRKTDGGTTDYAAPVKGRFTISRDDSKNT | | QTASITCSGDKLGDKYA |
| 2 | | LYLQMNSLKTEDTAVYYCTTEYSSSENFDYWGQ | | CWYQQKPGQSPVVVIYQ |
| | | GTLVTVSSASTKGPSVFPLAPSSKSTSGGTAAL | | DSKRPSGIPERFSGSNS |
| | | GCLVKDYFPEPVTVSWNSGALTSGVHTFPAVLQ | | GNTATLTISGTQAMDEA |
| | | SSGLYSLSSVVTVPSSSLGTQTYICNVNHKPSN | | DYYCQAWDSSTAVFGGG |
| | | TKVDKKVEPKSCDKTHTCPPCPAPEAAGGPSVF | | TKLTVLGQPKAAPSVTL |
| | | LFPPKPKDTLMISRTPEVTCVVVDVSHEDPEVK | | FPPSSEELQANKATLVC |
| | | FNWYVDGVEVHNAKTKPREEQYNSTYRVVSVLT | | LISDFYPGAVTVAWKAD |
| | | VLHQDWLNGKEYKCKVSNKALGAPIEKTISKAK | | SSPVKAGVETTTPSKQS |
| | | GQPREPQVYTLPPSREEMTKNQVSLTCLVKGFY | | NNKYAASSYLSLTPEQW |
| | | PSDIAVEWESNGQPENNYKTTPPVLDSDGSFFL | | KSHRSYSCQVTHEGSTV |
| | | YSKLTVDKSRWQQGNVFSCSVMHEALHNHYTQK | | EKTVAPTECS |
| | | SLSLSPGK | | |
| A115- | 5730 | MGWSCIILFLVATATGVHSEVQLVESGGGLVQP | 5918 | MGWSCIILFLVATATGV |
| LALA | | GGSLRLSCAASGFTFSSYWMHWVRQAPGKGLVW | | HSSYELTQPPSVSVSPG |
| PGhL | | VSRINSDGSSTSYADSVKGRFTISRDNAKNTLY | | QTASITCSGDKLGDKYA |
| 2 | | LQMNSLRAEDTAVYYCARDLGAARPRGFDYWGQ | | CWYQQKPGQSPVLVIYQ |
| | | GTLVTVSSASTKGPSVFPLAPSSKSTSGGTAAL | | DSKRPSGIPERFSGSNS |
| | | GCLVKDYFPEPVTVSWNSGALTSGVHTFPAVLQ | | GNTATLTISGTQAMDEA |
| | | SSGLYSLSSVVTVPSSSLGTQTYICNVNHKPSN | | DYYCQAWDSSTVVFGGG |
| | | TKVDKKVEPKSCDKTHTCPPCPAPEAAGGPSVF | | TKLTVLGQPKAAPSVTL |
| | | LFPPKPKDTLMISRTPEVTCVVVDVSHEDPEVK | | FPPSSEELQANKATLVC |
| | | FNWYVDGVEVHNAKTKPREEQYNSTYRVVSVLT | | LISDFYPGAVTVAWKAD |
| | | VLHQDWLNGKEYKCKVSNKALGAPIEKTISKAK | | SSPVKAGVETTTPSKQS |
| | | GQPREPQVYTLPPSREEMTKNQVSLTCLVKGFY | | NNKYAASSYLSLTPEQW |
| | | PSDIAVEWESNGQPENNYKTTPPVLDSDGSFFL | | KSHRSYSCQVTHEGSTV |
| | | YSKLTVDKSRWQQGNVFSCSVMHEALHNHYTQK | | EKTVAPTECS |
| | | SLSLSPGK | | |
| A116- | 5731 | MGWSCIILFLVATATGVHSEVQLVESGGGLVQP | 5919 | MGWSCIILFLVATATGV |
| LALA | | GGSLRLSCAASGFTFSSYAMSWVRQAPGKGLEW | | HSSYELTQPPSVSVSPG |
| PGhL | | VSAISGSGGSTYYADSVKGRFTISRDNSKNTLY | | QTARITCSGDALPKKYA |
| 2 | | LQMNSLRAEDTAVYYCAKEGDSGYDSAFDIWGQ | | YWYQQKSGQAPVLVIYE |
| | | GTMVTVSSASTKGPSVFPLAPSSKSTSGGTAAL | | DSKRPSGIPERFSGSSS |
| | | GCLVKDYFPEPVTVSWNSGALTSGVHTFPAVLQ | | GTMATLTISGAQVEDEA |
| | | SSGLYSLSSVVTVPSSSLGTQTYICNVNHKPSN | | DYYCYSTDSSGNRVEGG |
| | | TKVDKKVEPKSCDKTHTCPPCPAPEAAGGPSVF | | GTKLTVLGQPKAAPSVT |
| | | LFPPKPKDTLMISRTPEVTCVVVDVSHEDPEVK | | LFPPSSEELQANKATLV |
| | | FNWYVDGVEVHNAKTKPREEQYNSTYRVVSVLT | | CLISDFYPGAVTVAWKA |
| | | VLHQDWLNGKEYKCKVSNKALGAPIEKTISKAK | | DSSPVKAGVETTTPSKQ |
| | | GQPREPQVYTLPPSREEMTKNQVSLTCLVKGFY | | SNNKYAASSYLSLTPEQ |
| | | PSDIAVEWESNGQPENNYKTTPPVLDSDGSFFL | | WKSHRSYSCQVTHEGST |
| | | YSKLTVDKSRWQQGNVFSCSVMHEALHNHYTQK | | VEKTVAPTECS |
| | | SLSLSPGK | | |
| A117- | 5732 | MGWSCIILFLVATATGVHSQVQLQESGPGLVKP | 5920 | MGWSCIILFLVATATGV |
| LALA | | SGTLSLTCAVSGGSISSNNWWSWVRQPPGKGLE | | HSSYELTQPPSVSVSPG |
| PGhL | | WIGEIYHSGSTNYNPSLKSRVTISVDKSKNQFS | | QTARITCSGDALPKKYA |
| 2 | | LKLSSVTAADTAVYYCARVLNWNYGDAFDIWGQ | | YWYQQKSGQAPVLVIYE |
| | | GTMVTVSSASTKGPSVFPLAPSSKSTSGGTAAL | | DSKRPSGIPERFSGSSS |
| | | GCLVKDYFPEPVTVSWNSGALTSGVHTFPAVLQ | | GTMATLTISGAQVEDEA |
| | | SSGLYSLSSVVTVPSSSLGTQTYICNVNHKPSN | | DYYCYSTDSSGNHRGFG |
| | | TKVDKKVEPKSCDKTHTCPPCPAPEAAGGPSVF | | GGTKLTVLGQPKAAPSV |
| | | LFPPKPKDTLMISRTPEVTCVVVDVSHEDPEVK | | TLFPPSSEELQANKATL |
| | | FNWYVDGVEVHNAKTKPREEQYNSTYRVVSVLT | | VCLISDFYPGAVTVAWK |
| | | VLHQDWLNGKEYKCKVSNKALGAPIEKTISKAK | | ADSSPVKAGVETTTPSK |
| | | GQPREPQVYTLPPSREEMTKNQVSLTCLVKGFY | | QSNNKYAASSYLSLTPE |
| | | PSDIAVEWESNGQPENNYKTTPPVLDSDGSFFL | | QWKSHRSYSCQVTHEGS |
| | | YSKLTVDKSRWQQGNVFSCSVMHEALHNHYTQK | | TVEKTVAPTECS |
| | | SLSLSPGK | | |
| A118- | 5733 | MGWSCIILFLVATATGVHSQVQLQESGPGLVKP | 5921 | MGWSCIILFLVATATGV |
| LALA | | SGTLSLTCAVSGGSISSSNWWSWVRQPPGKGLE | | HSQSALTQPRSVSGSPG |
| PGhL | | WIGEIYHSGSTNYNPSLKSRVTISVDKSKNQFS | | QSVTISCTGTSSDVGGY |
| 2 | | LKLSSVTAADTAVYYCARDPSIVGATAFDIWGQ | | NYVSWYQQHPGKAPKLM |
| | | GTMVTVSSASTKGPSVFPLAPSSKSTSGGTAAL | | IYDVSKRPSGVPDRFSG |
| | | GCLVKDYFPEPVTVSWNSGALTSGVHTFPAVLQ | | SKSGNTASLTISGLQAE |
| | | SSGLYSLSSVVTVPSSSLGTQTYICNVNHKPSN | | DEADYYCCSYAQGVVFG |
| | | TKVDKKVEPKSCDKTHTCPPCPAPEAAGGPSVF | | GGTKLTVLGQPKAAPSV |
| | | LFPPKPKDTLMISRTPEVTCVVVDVSHEDPEVK | | TLFPPSSEELQANKATL |
| | | FNWYVDGVEVHNAKTKPREEQYNSTYRVVSVLT | | VCLISDFYPGAVTVAWK |
| | | VLHQDWLNGKEYKCKVSNKALGAPIEKTISKAK | | ADSSPVKAGVETTTPSK |
| | | GQPREPQVYTLPPSREEMTKNQVSLTCLVKGFY | | QSNNKYAASSYLSLTPE |
| | | PSDIAVEWESNGQPENNYKTTPPVLDSDGSFFL | | QWKSHRSYSCQVTHEGS |
| | | YSKLTVDKSRWQQGNVFSCSVMHEALHNHYTQK | | TVEKTVAPTECS |
| | | SLSLSPGK | | |
| A119- | 5734 | MGWSCIILFLVATATGVHSQVQLQESGPGLVKP | 5922 | MGWSCIILFLVATATGV |
| LALA | | SGTLSLTCAVSGGSIISSNWWSWVRQSPGKGLG | | HSSSELTQDPAVSVALG |
| PGhL | | WIGEIYHSGSTTYNPSLKSRVTISVDKSKNQFS | | QTVRITCQGDSLRSYYA |
| 2 | | LKLSSVTAADTALYYCARSHIVGVNGGFDYWGQ | | SWYQQKPGQAPVLLIYG |
| | | GTLVTVSSASTKGPSVFPLAPSSKSTSGGTAAL | | KNNRPSGIPDRFSGSSS |
| | | GCLVKDYFPEPVTVSWNSGALTSGVHTFPAVLQ | | GNTASLTITGAQAEDEA |
| | | SSGLYSLSSVVTVPSSSLGTQTYICNVNHKPSN | | DYYCNSRDSSGNHWVFG |
| | | TKVDKKVEPKSCDKTHTCPPCPAPEAAGGPSVF | | GGTKLTVLGQPKAAPSV |
| | | LFPPKPKDTLMISRTPEVTCVVVDVSHEDPEVK | | TLFPPSSEELQANKATL |
| | | FNWYVDGVEVHNAKTKPREEQYNSTYRVVSVLT | | VCLISDFYPGAVTVAWK |
| | | VLHQDWLNGKEYKCKVSNKALGAPIEKTISKAK | | ADSSPVKAGVETTTPSK |
| | | GQPREPQVYTLPPSREEMTKNQVSLTCLVKGFY | | QSNNKYAASSYLSLTPE |
| | | PSDIAVEWESNGQPENNYKTTPPVLDSDGSFFL | | QWKSHRSYSCQVTHEGS |
| | | YSKLTVDKSRWQQGNVFSCSVMHEALHNHYTQK | | TVEKTVAPTECS |
| | | SLSLSPGK | | |
| A120- | 5735 | MGWSCIILFLVATATGVHSEVQLVESGGGLVKP | 5923 | MGWSCIILFLVATATGV |
| LALA | | GGSLRLSCAASGFTFSSYSMNWVRQAPGKGLEW | | HSSSELTQDPAVSVALG |
| PGhL | | VSSISSSSSYIYYADSVKGRFTISRDNAKNSLY | | QTVRITCQGDSLRSYYA |
| 2 | | LQMNSLRAEDTAVYYCARDRYNWNYRAFDIWGQ | | SWYQQKPGQAPVLVIYG |
| | | GTMVTVSSASTKGPSVFPLAPSSKSTSGGTAAL | | KNNRPSGIPDRFSGSSS |
| | | GCLVKDYFPEPVTVSWNSGALTSGVHTFPAVLQ | | GNTASLTITGAQAEDEA |
| | | SSGLYSLSSVVTVPSSSLGTQTYICNVNHKPSN | | DYYCNSRDSSGNHLVFG |
| | | TKVDKKVEPKSCDKTHTCPPCPAPEAAGGPSVF | | TGTKVTVLGQPKAAPSV |
| | | LFPPKPKDTLMISRTPEVTCVVVDVSHEDPEVK | | TLFPPSSEELQANKATL |
| | | FNWYVDGVEVHNAKTKPREEQYNSTYRVVSVLT | | VCLISDFYPGAVTVAWK |
| | | VLHQDWLNGKEYKCKVSNKALGAPIEKTISKAK | | ADSSPVKAGVETTTPSK |
| | | GQPREPQVYTLPPSREEMTKNQVSLTCLVKGFY | | QSNNKYAASSYLSLTPE |
| | | PSDIAVEWESNGQPENNYKTTPPVLDSDGSFFL | | QWKSHRSYSCQVTHEGS |
| | | YSKLTVDKSRWQQGNVFSCSVMHEALHNHYTQK | | TVEKTVAPTECS |
| | | SLSLSPGK | | |
| A121- | 5736 | MGWSCIILFLVATATGVHSEVQLVESGGGLVQP | 5924 | MGWSCIILFLVATATGV |
| LALA | | GGSLRLSCAASGFTFSSYWMSWVRQAPGKGLEW | | HSSSELTQDPAVSVALG |
| PGhL | | VANIKQDGSEKYYVDSVKGRFTISRDNAKNSLY | | QTVRITCQGDSLRSYYA |
| 2 | | LQMNSLRAEDTAVYYCARDLGRGTISWFDPWGQ | | SWYQQKPGQAPVLVIYG |
| | | GTLVTVSSASTKGPSVFPLAPSSKSTSGGTAAL | | KNNRPSGIPDRFSGSSS |
| | | GCLVKDYFPEPVTVSWNSGALTSGVHTFPAVLQ | | GNTASLTITGAQAEDEA |
| | | SSGLYSLSSVVTVPSSSLGTQTYICNVNHKPSN | | DYYCNSRDSSGNHWVFG |
| | | TKVDKKVEPKSCDKTHTCPPCPAPEAAGGPSVE | | GGTKLTVLGQPKAAPSV |
| | | LFPPKPKDTLMISRTPEVTCVVVDVSHEDPEVK | | TLFPPSSEELQANKATL |
| | | FNWYVDGVEVHNAKTKPREEQYNSTYRVVSVLT | | VCLISDFYPGAVTVAWK |
| | | VLHQDWLNGKEYKCKVSNKALGAPIEKTISKAK | | ADSSPVKAGVETTTPSK |
| | | GQPREPQVYTLPPSREEMTKNQVSLTCLVKGFY | | QSNNKYAASSYLSLTPE |
| | | PSDIAVEWESNGQPENNYKTTPPVLDSDGSFFL | | QWKSHRSYSCQVTHEGS |
| | | YSKLTVDKSRWQQGNVFSCSVMHEALHNHYTQK | | TVEKTVAPTECS |
| | | SLSLSPGK | | |
| A122- | 5737 | MGWSCIILFLVATATGVHSQITLKESGPTLVKA | 5925 | MGWSCIILFLVATATGV |
| LALA | | TQTLTLTCTFSGFSLNSSGVGVVWIRQPPGKAL | | HSSYVLTQPPSVSVAPG |
| PGhL | | EWLALIYWNGDKRYSQSLKNRLTITEDTSKNQV | | KTARITCGGNNIGSKSV |
| 2 | | VLAMTNMDPVDTATYYCTQTGYDSRWSFAYWGQ | | HWYQQKPGQAPVLVIYY |
| | | GTLVTVSSASTKGPSVFPLAPSSKSTSGGTAAL | | DSDRPSGIPERFSGSNS |
| | | GCLVKDYFPEPVTVSWNSGALTSGVHTFPAVLQ | | GNTATLTISRVEAGDEA |
| | | SSGLYSLSSVVTVPSSSLGTQTYICNVNHKPSN | | DYYCQVWDSSSDHWVFG |
| | | TKVDKKVEPKSCDKTHTCPPCPAPEAAGGPSVF | | GGTKLTVLGQPKAAPSV |
| | | LFPPKPKDTLMISRTPEVTCVVVDVSHEDPEVK | | TLFPPSSEELQANKATL |
| | | FNWYVDGVEVHNAKTKPREEQYNSTYRVVSVLT | | VCLISDFYPGAVTVAWK |
| | | VLHQDWLNGKEYKCKVSNKALGAPIEKTISKAK | | ADSSPVKAGVETTTPSK |
| | | GQPREPQVYTLPPSREEMTKNQVSLTCLVKGFY | | QSNNKYAASSYLSLTPE |
| | | PSDIAVEWESNGQPENNYKTTPPVLDSDGSFFL | | QWKSHRSYSCQVTHEGS |
| | | YSKLTVDKSRWQQGNVFSCSVMHEALHNHYTQK | | TVEKTVAPTECS |
| | | SLSLSPGK | | |
| A123- | 5738 | MGWSCIILFLVATATGVHSQVQLVQSGAEVKKP | 5926 | MGWSCIILFLVATATGV |
| LALA | | GASVKVSCKTSGYIFMNYGITWVRHAPGQGLEW | | HSSSELTQDPAVSVALG |
| PGhL | | MGWISAYNGNTNYAQKVQGRVTMTTDTSTSTAN | | QTVRITCQGDSLRSYYA |
| 2 | | MELRSLRSDDTAVYYCAREGQWRGRGWFALWGQ | | SWYQQKPGQAPVLVIYG |
| | | GTTVTVSSASTKGPSVFPLAPSSKSTSGGTAAL | | KNNRPSGIPDRFSGSSS |
| | | GCLVKDYFPEPVTVSWNSGALTSGVHTFPAVLQ | | GNTASLTITGAQAEDEA |
| | | SSGLYSLSSVVTVPSSSLGTQTYICNVNHKPSN | | DYYCNSRDSSGNHLVFG |
| | | TKVDKKVEPKSCDKTHTCPPCPAPEAAGGPSVF | | GGTKLTVLGQPKAAPSV |
| | | LFPPKPKDTLMISRTPEVTCVVVDVSHEDPEVK | | TLFPPSSEELQANKATL |
| | | FNWYVDGVEVHNAKTKPREEQYNSTYRVVSVLT | | VCLISDFYPGAVTVAWK |
| | | VLHQDWLNGKEYKCKVSNKALGAPIEKTISKAK | | ADSSPVKAGVETTTPSK |
| | | GQPREPQVYTLPPSREEMTKNQVSLTCLVKGFY | | QSNNKYAASSYLSLTPE |
| | | PSDIAVEWESNGQPENNYKTTPPVLDSDGSFFL | | QWKSHRSYSCQVTHEGS |
| | | YSKLTVDKSRWQQGNVFSCSVMHEALHNHYTQK | | TVEKTVAPTECS |
| | | SLSLSPGK | | |
| A124- | 5739 | MGWSCIILFLVATATGVHSQVQLVQSGSELKKP | 5927 | MGWSCIILFLVATATGV |
| LALA | | GASVKVSCKASGYTFTNYAMNWVRQAPGQGLEW | | HSSSELTQDPAVSVALG |
| PGhL | | MGWINTNTGKPTYAQGFTGRFVFSLDTSVSTAH | | QTVRITCQGDSLRSYYA |
| 2 | | LQISGLKAEDTAVYYCARERYFEDFHYMDVWGK | | SWYQQKPGQAPVLVIYG |
| | | GTTVTVSSASTKGPSVFPLAPSSKSTSGGTAAL | | KNNRPSGIPDRFSGSSS |
| | | GCLVKDYFPEPVTVSWNSGALTSGVHTFPAVLQ | | GNTASLTITGAQAEDEA |
| | | SSGLYSLSSVVTVPSSSLGTQTYICNVNHKPSN | | DYYCNSRDSSGNHLVFG |
| | | TKVDKKVEPKSCDKTHTCPPCPAPEAAGGPSVE | | GGTKLTVLGQPKAAPSV |
| | | LFPPKPKDTLMISRTPEVTCVVVDVSHEDPEVK | | TLFPPSSEELQANKATL |
| | | FNWYVDGVEVHNAKTKPREEQYNSTYRVVSVLT | | VCLISDFYPGAVTVAWK |
| | | VLHQDWLNGKEYKCKVSNKALGAPIEKTISKAK | | ADSSPVKAGVETTTPSK |
| | | GQPREPQVYTLPPSREEMTKNQVSLTCLVKGFY | | QSNNKYAASSYLSLTPE |
| | | PSDIAVEWESNGQPENNYKTTPPVLDSDGSFFL | | QWKSHRSYSCQVTHEGS |
| | | YSKLTVDKSRWQQGNVFSCSVMHEALHNHYTQK | | TVEKTVAPTECS |
| | | SLSLSPGK | | |
| A125- | 5740 | MGWSCIILFLVATATGVHSQVQLVQSGAEVKKP | 5928 | MGWSCIILFLVATATGV |
| LALA | | GASVKVSCKASGYTFTDNYIHWVRRAPGQGLEW | | HSSSELTQDPAVSVALG |
| PGhL | | MGWLNPNSGGTNFAQKFQGRVTMTRDTSISSVY | | QTVRITCQGDSLRSYYA |
| 2 | | MILSSLRSDDTAVYYCARSSWLQLTYYFDYWGQ | | SWYQQKPGQAPVLVIYG |
| | | GTLVTVSSASTKGPSVFPLAPSSKSTSGGTAAL | | KNNRPSGIPDRFSGSNS |
| | | GCLVKDYFPEPVTVSWNSGALTSGVHTFPAVLQ | | GNTASLTITGAQAEDEA |
| | | SSGLYSLSSVVTVPSSSLGTQTYICNVNHKPSN | | DYYCNSRDSSGNHLLFG |
| | | TKVDKKVEPKSCDKTHTCPPCPAPEAAGGPSVF | | GGTKLTVLGQPKAAPSV |
| | | LFPPKPKDTLMISRTPEVTCVVVDVSHEDPEVK | | TLFPPSSEELQANKATL |
| | | FNWYVDGVEVHNAKTKPREEQYNSTYRVVSVLT | | VCLISDFYPGAVTVAWK |
| | | VLHQDWLNGKEYKCKVSNKALGAPIEKTISKAK | | ADSSPVKAGVETTTPSK |
| | | GQPREPQVYTLPPSREEMTKNQVSLTCLVKGFY | | QSNNKYAASSYLSLTPE |
| | | PSDIAVEWESNGQPENNYKTTPPVLDSDGSFFL | | QWKSHRSYSCQVTHEGS |
| | | YSKLTVDKSRWQQGNVFSCSVMHEALHNHYTQK | | TVEKTVAPTECS |
| | | SLSLSPGK | | |
| A126- | 5741 | MGWSCIILFLVATATGVHSQVQLVQSGAEVKKP | 5929 | MGWSCIILFLVATATGV |
| LALA | | GASVKVSCKASGYTFTSYYIHWVRQAPGQGLEW | | HSSSELTQDPGVSVALG |
| PGhL | | MGIINPSGGSTSYAQKFQGRVTMTRDTSTSTVY | | QTVRITCQGDSLRSYYA |
| 2 | | MEVSSLRSEDTAVYYCAREGLQLGSNWFDPWGQ | | SWYQQKPGQAPVLVMYG |
| | | GTLVTVSSASTKGPSVFPLAPSSKSTSGGTAAL | | KKNRPSGIPDRESGSSS |
| | | GCLVKDYFPEPVTVSWNSGALTSGVHTFPAVLQ | | GNTASLTITGAQAEDEA |
| | | SSGLYSLSSVVTVPSSSLGTQTYICNVNHKPSN | | DYYCKSRDSSGNHVVFG |
| | | TKVDKKVEPKSCDKTHTCPPCPAPEAAGGPSVE | | GGTKLTVLGQPKAAPSV |
| | | LFPPKPKDTLMISRTPEVTCVVVDVSHEDPEVK | | TLFPPSSEELQANKATL |
| | | FNWYVDGVEVHNAKTKPREEQYNSTYRVVSVLT | | VCLISDFYPGAVTVAWK |
| | | VLHQDWLNGKEYKCKVSNKALGAPIEKTISKAK | | ADSSPVKAGVETTTPSK |
| | | GQPREPQVYTLPPSREEMTKNQVSLTCLVKGFY | | QSNNKYAASSYLSLTPE |
| | | PSDIAVEWESNGQPENNYKTTPPVLDSDGSFEL | | QWKSHRSYSCQVTHEGS |
| | | YSKLTVDKSRWQQGNVFSCSVMHEALHNHYTQK | | TVEKTVAPTECS |
| | | SLSLSPGK | | |
| A127- | 5742 | MGWSCIILFLVATATGVHSEVQLVESGGGLVQP | 5930 | MGWSCIILFLVATATGV |
| LALA | | GGSLRLSCAASGFTFSSYSMNWVRQAPGKGLEW | | HSSYELTQPPSVSVSPG |
| PGhL | | VSYISSSSSTIYYADSVKGRFTISRDNAKNSLY | | QTARITCSGDALPKKYA |
| 2 | | LQMNSLRDEDTAVYYCARNDILTGEDAFDIWGQ | | YWYQQKSGQAPVLVIYE |
| | | GTMVTVSSASTKGPSVFPLAPSSKSTSGGTAAL | | DSKRPSGIPERFSGSSS |
| | | GCLVKDYFPEPVTVSWNSGALTSGVHTFPAVLQ | | GTMATLTISGAQVEDEA |
| | | SSGLYSLSSVVTVPSSSLGTQTYICNVNHKPSN | | DYYCYSTDSSGNHRVFG |
| | | TKVDKKVEPKSCDKTHTCPPCPAPEAAGGPSVF | | GGTKLTVLGQPKAAPSV |
| | | LFPPKPKDTLMISRTPEVTCVVVDVSHEDPEVK | | TLFPPSSEELQANKATL |
| | | FNWYVDGVEVHNAKTKPREEQYNSTYRVVSVLT | | VCLISDFYPGAVTVAWK |
| | | VLHQDWLNGKEYKCKVSNKALGAPIEKTISKAK | | ADSSPVKAGVETTTPSK |
| | | GQPREPQVYTLPPSREEMTKNQVSLTCLVKGFY | | QSNNKYAASSYLSLTPE |
| | | PSDIAVEWESNGQPENNYKTTPPVLDSDGSFFL | | QWKSHRSYSCQVTHEGS |
| | | YSKLTVDKSRWQQGNVFSCSVMHEALHNHYTQK | | TVEKTVAPTECS |
| | | SLSLSPGK | | |
| A128- | 5743 | MGWSCIILFLVATATGVHSEVQLVESGGGLVQP | 5931 | MGWSCIILFLVATATGV |
| LALA | | GGSLRLSCAASGFTFSNYAMSWVRQAPGKGLGW | | HSSSELTQDPAVSVALG |
| PGhL | | VSGISGSGGRTYYADSVKGRFTISRDNSKNTLY | | QTVRITCQGDSFRNYYA |
| 2 | | LQMNSLRAEDTAVYYCAKESIIVGATMFDYWGQ | | SWYQQMPGQAPVLVIYG |
| | | GTLVTVSSASTKGPSVFPLAPSSKSTSGGTAAL | | KNNRPSGIPDRFSGSSS |
| | | GCLVKDYFPEPVTVSWNSGALTSGVHTFPAVLQ | | GNTASLTITGAQAEDEA |
| | | SSGLYSLSSVVTVPSSSLGTQTYICNVNHKPSN | | DYYCNSRDSSGNHWVFG |
| | | TKVDKKVEPKSCDKTHTCPPCPAPEAAGGPSVF | | GGTKLTVLGQPKAAPSV |
| | | LFPPKPKDTLMISRTPEVTCVVVDVSHEDPEVK | | TLFPPSSEELQANKATL |
| | | FNWYVDGVEVHNAKTKPREEQYNSTYRVVSVLT | | VCLISDFYPGAVTVAWK |
| | | VLHQDWLNGKEYKCKVSNKALGAPIEKTISKAK | | ADSSPVKAGVETTTPSK |
| | | GQPREPQVYTLPPSREEMTKNQVSLTCLVKGFY | | QSNNKYAASSYLSLTPE |
| | | PSDIAVEWESNGQPENNYKTTPPVLDSDGSFFL | | QWKSHRSYSCQVTHEGS |
| | | YSKLTVDKSRWQQGNVFSCSVMHEALHNHYTQK | | TVEKTVAPTECS |
| | | SLSLSPGK | | |
| A129- | 5744 | MGWSCIILFLVATATGVHSQVQLVESGGGVVQP | 5932 | MGWSCIILFLVATATGV |
| LALA | | GRSLRLSCAASGFTFSSYGMHWVRQAPGKGLEW | | HSSSELTQDPAVSVALG |
| PGhL | | VAVISYDGSNKYYADSVKGRFTISRDNSKNTLY | | QTVRITCQGDSLRSYYA |
| 2 | | LQMNSLRAEDTAVYYCAKGIAALGYYYMDVWGK | | SWYQQKPGQAPVLVIYG |
| | | GTTVTVSSASTKGPSVFPLAPSSKSTSGGTAAL | | KNNRPSGIPDRFSGSSS |
| | | GCLVKDYFPEPVTVSWNSGALTSGVHTFPAVLQ | | GNTASLTITGAQAEDEA |
| | | SSGLYSLSSVVTVPSSSLGTQTYICNVNHKPSN | | DYYCNSRDSSGNHLVFG |
| | | TKVDKKVEPKSCDKTHTCPPCPAPEAAGGPSVE | | GGTKLTVLGQPKAAPSV |
| | | LFPPKPKDTLMISRTPEVTCVVVDVSHEDPEVK | | TLFPPSSEELQANKATL |
| | | FNWYVDGVEVHNAKTKPREEQYNSTYRVVSVLT | | VCLISDFYPGAVTVAWK |
| | | VLHQDWLNGKEYKCKVSNKALGAPIEKTISKAK | | ADSSPVKAGVETTTPSK |
| | | GQPREPQVYTLPPSREEMTKNQVSLTCLVKGFY | | QSNNKYAASSYLSLTPE |
| | | PSDIAVEWESNGQPENNYKTTPPVLDSDGSFFL | | QWKSHRSYSCQVTHEGS |
| | | YSKLTVDKSRWQQGNVFSCSVMHEALHNHYTQK | | TVEKTVAPTECS |
| | | SLSLSPGK | | |
| A130- | 5745 | MGWSCIILFLVATATGVHSEVQLVQSGAEMKKP | 5933 | MGWSCIILFLVATATGV |
| LALA | | GESLKISCKDSGYRFSNYWIGWVRQLPGKGLEW | | HSSSELTQDPAVSVALG |
| PGhL | | MGIIYPGDSDTRYSPSFQGQVTISADKSINTAY | | QTVRITCQGDSLRSYYA |
| 2 | | LQWNSLKASDTAIYYCAKRRITGSHNWFDPWGQ | | SWYQQKPGQAPVLVIYG |
| | | GTLVTVSSASTKGPSVFPLAPSSKSTSGGTAAL | | KNNRPSGIPDRFSGSSS |
| | | GCLVKDYFPEPVTVSWNSGALTSGVHTFPAVLQ | | GNTASLTITGAQAEDEA |
| | | SSGLYSLSSVVTVPSSSLGTQTYICNVNHKPSN | | DYYCNSRDSSGNHLVFG |
| | | TKVDKKVEPKSCDKTHTCPPCPAPEAAGGPSVF | | GGTKLTVLGQPKAAPSV |
| | | LFPPKPKDTLMISRTPEVTCVVVDVSHEDPEVK | | TLFPPSSEELQANKATL |
| | | FNWYVDGVEVHNAKTKPREEQYNSTYRVVSVLT | | VCLISDFYPGAVTVAWK |
| | | VLHQDWLNGKEYKCKVSNKALGAPIEKTISKAK | | ADSSPVKAGVETTTPSK |
| | | GQPREPQVYTLPPSREEMTKNQVSLTCLVKGFY | | QSNNKYAASSYLSLTPE |
| | | PSDIAVEWESNGQPENNYKTTPPVLDSDGSFFL | | QWKSHRSYSCQVTHEGS |
| | | YSKLTVDKSRWQQGNVFSCSVMHEALHNHYTQK | | TVEKTVAPTECS |
| | | SLSLSPGK | | |
| A131- | 5746 | MGWSCIILFLVATATGVHSQVQLVQSGSELKKP | 5934 | MGWSCIILFLVATATGV |
| LALA | | GASVKVSCKASGYTFTSYAMNWVRQAPGQGLEW | | HSQTVVTQEPSLTVSPG |
| PGhL | | MGWINTNTGNPTYAQGFTGRFVFSLDTSVSTAY | | GTVTLTCASSTGAVTSG |
| 2 | | LQISSLKAEDTAVYYCARGGTIFGVVNFDYWGQ | | YYPNWFQQKPGQAPRAL |
| | | GTLVTVSSASTKGPSVFPLAPSSKSTSGGTAAL | | IYSTSNKHSWTPARFSG |
| | | GCLVKDYFPEPVTVSWNSGALTSGVHTFPAVLQ | | SLLGGKAALTLSGVQPE |
| | | SSGLYSLSSVVTVPSSSLGTQTYICNVNHKPSN | | DEAEYYCLLYYGGARVE |
| | | TKVDKKVEPKSCDKTHTCPPCPAPEAAGGPSVF | | GGGTKLTVLGQPKAAPS |
| | | LFPPKPKDTLMISRTPEVTCVVVDVSHEDPEVK | | VTLFPPSSEELQANKAT |
| | | FNWYVDGVEVHNAKTKPREEQYNSTYRVVSVLT | | LVCLISDFYPGAVTVAW |
| | | VLHQDWLNGKEYKCKVSNKALGAPIEKTISKAK | | KADSSPVKAGVETTTPS |
| | | GQPREPQVYTLPPSREEMTKNQVSLTCLVKGFY | | KQSNNKYAASSYLSLTP |
| | | PSDIAVEWESNGQPENNYKTTPPVLDSDGSFFL | | EQWKSHRSYSCQVTHEG |
| | | YSKLTVDKSRWQQGNVFSCSVMHEALHNHYTQK | | STVEKTVAPTECS |
| | | SLSLSPGK | | |
| A132- | 5747 | MGWSCIILFLVATATGVHSQVQLVESGGGVVQP | 5935 | MGWSCIILFLVATATGV |
| LALA | | GRSLRLSCAASGFTFSSYVMHWVRQAPGKGLEW | | HSQSALTQPASVSGSPG |
| PGhL | | VAVIWYDGSNKYFADSVKGRFTISRDNSNNTLY | | QSITISCTGTSSDVGVY |
| 2 | | LQMNSLRAEDTGVYYCLSRSGYSAHNDGDYWGQ | | NFVSWYQQHPGKAPKLM |
| | | GTLVTVSSASTKGPSVFPLAPSSKSTSGGTAAL | | IYDVTKRPSGASERFSG |
| | | GCLVKDYFPEPVTVSWNSGALTSGVHTFPAVLQ | | SKSGSTASLTISGLQAE |
| | | SSGLYSLSSVVTVPSSSLGTQTYICNVNHKPSN | | DEADYYCCSYAGSSTWV |
| | | TKVDKKVEPKSCDKTHTCPPCPAPEAAGGPSVF | | FGGGTKLTVLGQPKAAP |
| | | LFPPKPKDTLMISRTPEVTCVVVDVSHEDPEVK | | SVTLFPPSSEELQANKA |
| | | FNWYVDGVEVHNAKTKPREEQYNSTYRVVSVLT | | TLVCLISDFYPGAVTVA |
| | | VLHQDWLNGKEYKCKVSNKALGAPIEKTISKAK | | WKADSSPVKAGVETTTP |
| | | GQPREPQVYTLPPSREEMTKNQVSLTCLVKGFY | | SKQSNNKYAASSYLSLT |
| | | PSDIAVEWESNGQPENNYKTTPPVLDSDGSFFL | | PEQWKSHRSYSCQVTHE |
| | | YSKLTVDKSRWQQGNVFSCSVMHEALHNHYTQK | | GSTVEKTVAPTECS |
| | | SLSLSPGK | | |
| A133- | 5748 | MGWSCIILFLVATATGVHSQVQLVQSGAEVKKP | 5936 | MGWSCIILFLVATATGV |
| LALA | | GASVKVSCKASGYTFTGYYIFWVRQAPGQGLEW | | HSQSVLTQPPSVSGAPG |
| PGhL | | MGWINPNSGGTNYAQKFQGRVTMTRDTSITTAY | | QRVTISCTGSSSNIGAG |
| 2 | | MELSRLRHDDTAVYYCTKEGLVVRPDWEDPWGQ | | YDVHWYQQLPGTAPKIL |
| | | GTLVTVSSASTKGPSVFPLAPSSKSTSGGTAAL | | IYVNNNRPSGVPDRESG |
| | | GCLVKDYFPEPVTVSWNSGALTSGVHTFPAVLQ | | SKSGTSASLAITGLQAE |
| | | SSGLYSLSSVVTVPSSSLGTQTYICNVNHKPSN | | DEADYYCQSYDSSLSGP |
| | | TKVDKKVEPKSCDKTHTCPPCPAPEAAGGPSVF | | VFGGGTKLTVLGQPKAA |
| | | LFPPKPKDTLMISRTPEVTCVVVDVSHEDPEVK | | PSVTLFPPSSEELQANK |
| | | FNWYVDGVEVHNAKTKPREEQYNSTYRVVSVLT | | ATLVCLISDFYPGAVTV |
| | | VLHQDWLNGKEYKCKVSNKALGAPIEKTISKAK | | AWKADSSPVKAGVETTT |
| | | GQPREPQVYTLPPSREEMTKNQVSLTCLVKGFY | | PSKQSNNKYAASSYLSL |
| | | PSDIAVEWESNGQPENNYKTTPPVLDSDGSFFL | | TPEQWKSHRSYSCQVTH |
| | | YSKLTVDKSRWQQGNVFSCSVMHEALHNHYTQK | | EGSTVEKTVAPTECS |
| | | SLSLSPGK | | |
| A134- | 5749 | MGWSCIILFLVATATGVHSQVQLQQSGPGLVKP | 5937 | MGWSCIILFLVATATGV |
| LALA | | TQTLSLTCAISGDSVSSNSAAWNWIRQSPSRGL | | HSQSALTQPASVSGSPG |
| PGhL | | EWLGRTYYRSKWYNDYAVSVRSRITINPDTSKN | | QSITISCTGTSSDVGGY |
| 2 | | QFSLHLNSVTPEDTAVYYCARKGRDVFDIWGQG | | NYVSWYQQHPGKAPKLM |
| | | TMVTVSSASTKGPSVFPLAPSSKSTSGGTAALG | | IYDVSKRPSGVSNRFSG |
| | | CLVKDYFPEPVTVSWNSGALTSGVHTFPAVLQS | | SKSGNTASLTISGLQAE |
| | | SGLYSLSSVVTVPSSSLGTQTYICNVNHKPSNT | | DETDYYCCSYAGSSTYW |
| | | KVDKKVEPKSCDKTHTCPPCPAPEAAGGPSVFL | | VFGGGTKLTVLGQPKAA |
| | | FPPKPKDTLMISRTPEVTCVVVDVSHEDPEVKF | | PSVTLFPPSSEELQANK |
| | | NWYVDGVEVHNAKTKPREEQYNSTYRVVSVLTV | | ATLVCLISDFYPGAVTV |
| | | LHQDWLNGKEYKCKVSNKALGAPIEKTISKAKG | | AWKADSSPVKAGVETTT |
| | | QPREPQVYTLPPSREEMTKNQVSLTCLVKGFYP | | PSKQSNNKYAASSYLSL |
| | | SDIAVEWESNGQPENNYKTTPPVLDSDGSFFLY | | TPEQWKSHRSYSCQVTH |
| | | SKLTVDKSRWQQGNVFSCSVMHEALHNHYTQKS | | EGSTVEKTVAPTECS |
| | | LSLSPGK | | |
| A135- | 5750 | MGWSCIILFLVATATGVHSQVQLVQSGAEVKKP | 5938 | MGWSCIILFLVATATGV |
| LALA | | GASVKVSCKASGYTFTSYGISWVRQAPGQGLEW | | HSQSVLTQPPSVSGAPG |
| PGhL | | MGWISAYNGNTNYAQKLQGRVTMTTDTSTSTAY | | QRVTISCTGSSSNIGAG |
| 2 | | MELRSLRSDDTAVYYCAREGSGSYSDAFDIWGQ | | YDVHWYQQLPGTAPKLL |
| | | GTMVTVSSASTKGPSVFPLAPSSKSTSGGTAAL | | IYGNSNRPSGVPDRESG |
| | | GCLVKDYFPEPVTVSWNSGALTSGVHTFPAVLQ | | SKSGTSASLAITGLQAE |
| | | SSGLYSLSSVVTVPSSSLGTQTYICNVNHKPSN | | DEADYYCQSYDSSLSGS |
| | | TKVDKKVEPKSCDKTHTCPPCPAPEAAGGPSVF | | YVFGTGTKVTVLGQPKA |
| | | LFPPKPKDTLMISRTPEVTCVVVDVSHEDPEVK | | APSVTLFPPSSEELQAN |
| | | FNWYVDGVEVHNAKTKPREEQYNSTYRVVSVLT | | KATLVCLISDFYPGAVT |
| | | VLHQDWLNGKEYKCKVSNKALGAPIEKTISKAK | | VAWKADSSPVKAGVETT |
| | | GQPREPQVYTLPPSREEMTKNQVSLTCLVKGFY | | TPSKQSNNKYAASSYLS |
| | | PSDIAVEWESNGQPENNYKTTPPVLDSDGSFFL | | LTPEQWKSHRSYSCQVT |
| | | YSKLTVDKSRWQQGNVFSCSVMHEALHNHYTQK | | HEGSTVEKTVAPTECS |
| | | SLSLSPGK | | |
| A136- | 5751 | MGWSCIILFLVATATGVHSQITLKESGPTLVKP | 5939 | MGWSCIILFLVATATGV |
| LALA | | TQTLTLTCTFSGFSITTSGVGVGWIRQPSGKAL | | HSQAVLTQPASLSASPG |
| PGhL | | EWLALIYWNDDKRYSPSLKSRLTITKDTSKNQV | | ASASLTCTLRSGIHVDT |
| 2 | | VLTMTNMDPVDTATYYCTHTEYRNTWCVDYWGQ | | SRIYWYQQKPGSPPQYL |
| | | GTLVTVSSASTKGPSVFPLAPSSKSTSGGTAAL | | LRYKSDSDKHQDSGVPS |
| | | GCLVKDYFPEPVTVSWNSGALTSGVHTFPAVLQ | | RFSGSKDASTNAGILLI |
| | | SSGLYSLSSVVTVPSSSLGTQTYICNVNHKPSN | | SGLQSEDEADYYCMIWH |
| | | TKVDKKVEPKSCDKTHTCPPCPAPEAAGGPSVF | | SSAIVFGGGTKLTVLGQ |
| | | LFPPKPKDTLMISRTPEVTCVVVDVSHEDPEVK | | PKAAPSVTLFPPSSEEL |
| | | FNWYVDGVEVHNAKTKPREEQYNSTYRVVSVLT | | QANKATLVCLISDFYPG |
| | | VLHQDWLNGKEYKCKVSNKALGAPIEKTISKAK | | AVTVAWKADSSPVKAGV |
| | | GQPREPQVYTLPPSREEMTKNQVSLTCLVKGFY | | ETTTPSKQSNNKYAASS |
| | | PSDIAVEWESNGQPENNYKTTPPVLDSDGSFFL | | YLSLTPEQWKSHRSYSC |
| | | YSKLTVDKSRWQQGNVFSCSVMHEALHNHYTQK | | QVTHEGSTVEKTVAPTE |
| | | SLSLSPGK | | CS |
| A137- | 5752 | MGWSCIILFLVATATGVHSQITLKESGPTLVKP | 5940 | MGWSCIILFLVATATGV |
| LALA | | TQTLTLTCTFSGFSLSTSGVGVGWIRQPPGKAL | | HSQAVLTQPASLSASPG |
| PGhL | | EWLALIYWNDDKRYSPSLKSRLTITKDTSKNQV | | ASASLTCTLRSGINVGS |
| 2 | | VLTMTNMDPVDTATYYCAHSPYTSGWPFDYWGQ | | YRIYWFQQRPGSPPQYL |
| | | GTLVTVSSASTKGPSVFPLAPSSKSTSGGTAAL | | LRYKSDSDKQQGSGVPS |
| | | GCLVKDYFPEPVTVSWNSGALTSGVHTFPAVLQ | | RFSGSKDASANAGILLI |
| | | SSGLYSLSSVVTVPSSSLGTQTYICNVNHKPSN | | SGLQSEDEADYYCMIWH |
| | | TKVDKKVEPKSCDKTHTCPPCPAPEAAGGPSVF | | SSASVFGGGTKVTVLGQ |
| | | LFPPKPKDTLMISRTPEVTCVVVDVSHEDPEVK | | PKAAPSVTLFPPSSEEL |
| | | FNWYVDGVEVHNAKTKPREEQYNSTYRVVSVLT | | QANKATLVCLISDFYPG |
| | | VLHQDWLNGKEYKCKVSNKALGAPIEKTISKAK | | AVTVAWKADSSPVKAGV |
| | | GQPREPQVYTLPPSREEMTKNQVSLTCLVKGFY | | ETTTPSKQSNNKYAASS |
| | | PSDIAVEWESNGQPENNYKTTPPVLDSDGSFFL | | YLSLTPEQWKSHRSYSC |
| | | YSKLTVDKSRWQQGNVFSCSVMHEALHNHYTQK | | QVTHEGSTVEKTVAPTE |
| | | SLSLSPGK | | CS |
| A138- | 5753 | MGWSCIILFLVATATGVHSQVQLVQSGAEVKKP | 5941 | MGWSCIILFLVATATGV |
| LALA | | GASVKVSCKASGYTFTSYDINWVRQATGQGLEW | | HSQAVLTQPASLSASPG |
| PGhL | | MGWMNPNSGNTGYAQKFQGRVTMTRNTSISTAY | | ASASLTCTLRSGINVGT |
| 2 | | MELSSLRSEDTAVYYCARVSYSSSWSLFDYWGQ | | YRIYWYQQKPGSPPQYL |
| | | GTLVTVSSASTKGPSVFPLAPSSKSTSGGTAAL | | LRYKSDSDKQQGSGVPS |
| | | GCLVKDYFPEPVTVSWNSGALTSGVHTFPAVLQ | | RFSGSKDASANAGILLI |
| | | SSGLYSLSSVVTVPSSSLGTQTYICNVNHKPSN | | SGLQSEDEADYYCMIWH |
| | | TKVDKKVEPKSCDKTHTCPPCPAPEAAGGPSVE | | SSAWVFGGGTKLTVLGQ |
| | | LFPPKPKDTLMISRTPEVTCVVVDVSHEDPEVK | | PKAAPSVTLFPPSSEEL |
| | | FNWYVDGVEVHNAKTKPREEQYNSTYRVVSVLT | | QANKATLVCLISDFYPG |
| | | VLHQDWLNGKEYKCKVSNKALGAPIEKTISKAK | | AVTVAWKADSSPVKAGV |
| | | GQPREPQVYTLPPSREEMTKNQVSLTCLVKGFY | | ETTTPSKQSNNKYAASS |
| | | PSDIAVEWESNGQPENNYKTTPPVLDSDGSFFL | | YLSLTPEQWKSHRSYSC |
| | | YSKLTVDKSRWQQGNVFSCSVMHEALHNHYTQK | | QVTHEGSTVEKTVAPTE |
| | | SLSLSPGK | | CS |
| A139- | 5754 | MGWSCIILFLVATATGVHSQVTLRESGPALVKP | 5942 | MGWSCIILFLVATATGV |
| LALA | | TQTLTLTCTFSGFSLSTSGMSLSWIRQPPGKAL | | HSSSELTQDPAVSVALG |
| PGhL | | EWLALIDWDDDQYYSTSLKTRLTISKDTSKNQV | | QTVRITCQGDSLRSYYA |
| 2 | | VLSMTNMDPVDTATYYCARIRGVGALDGFDFWG | | SWYQQKPGQAPVLVIYG |
| | | QGTMVTVSSASTKGPSVFPLAPSSKSTSGGTAA | | KNNRPSGIPDRFSGSSS |
| | | LGCLVKDYFPEPVTVSWNSGALTSGVHTFPAVL | | GNTASLTITGAQAEDEA |
| | | QSSGLYSLSSVVTVPSSSLGTQTYICNVNHKPS | | DYYCNSRDSSGNHLVFG |
| | | NTKVDKKVEPKSCDKTHTCPPCPAPEAAGGPSV | | GGTKLTVLGQPKAAPSV |
| | | FLFPPKPKDTLMISRTPEVTCVVVDVSHEDPEV | | TLFPPSSEELQANKATL |
| | | KFNWYVDGVEVHNAKTKPREEQYNSTYRVVSVL | | VCLISDFYPGAVTVAWK |
| | | TVLHQDWLNGKEYKCKVSNKALGAPIEKTISKA | | ADSSPVKAGVETTTPSK |
| | | KGQPREPQVYTLPPSREEMTKNQVSLTCLVKGF | | QSNNKYAASSYLSLTPE |
| | | YPSDIAVEWESNGQPENNYKTTPPVLDSDGSFF | | QWKSHRSYSCQVTHEGS |
| | | LYSKLTVDKSRWQQGNVFSCSVMHEALHNHYTQ | | TVEKTVAPTECS |
| | | KSLSLSPGK | | |
| A140- | 5755 | MGWSCIILFLVATATGVHSQVQLVQSGAEVKKP | 5943 | MGWSCIILFLVATATGV |
| LALA | | GASVKVSCKASGYTFTSYGISWVRQAPGQGLEW | | HSSSELTQDPAVSVALG |
| PGhL | | MGWISGYKGNTNCAQELQGRVTITSDTSTSTAY | | QTVRITCQGDSLRSYYA |
| 2 | | MELRSLRSDDTAVYYCARPLDYGDYEGWFDPWG | | SWYQQKPGQAPVLVIYG |
| | | QGTLVTVSSASTKGPSVFPLAPSSKSTSGGTAA | | KNNRPSGIPDRFSGSSS |
| | | LGCLVKDYFPEPVTVSWNSGALTSGVHTFPAVL | | GNTASLTITGAQAEDEA |
| | | QSSGLYSLSSVVTVPSSSLGTQTYICNVNHKPS | | DYYCNSRDSSGNHLVFG |
| | | NTKVDKKVEPKSCDKTHTCPPCPAPEAAGGPSV | | GGTKLTVLGQPKAAPSV |
| | | FLFPPKPKDTLMISRTPEVTCVVVDVSHEDPEV | | TLFPPSSEELQANKATL |
| | | KFNWYVDGVEVHNAKTKPREEQYNSTYRVVSVL | | VCLISDFYPGAVTVAWK |
| | | TVLHQDWLNGKEYKCKVSNKALGAPIEKTISKA | | ADSSPVKAGVETTTPSK |
| | | KGQPREPQVYTLPPSREEMTKNQVSLTCLVKGF | | QSNNKYAASSYLSLTPE |
| | | YPSDIAVEWESNGQPENNYKTTPPVLDSDGSFF | | QWKSHRSYSCQVTHEGS |
| | | LYSKLTVDKSRWQQGNVFSCSVMHEALHNHYTQ | | TVEKTVAPTECS |
| | | KSLSLSPGK | | |
| A141- | 5756 | MGWSCIILFLVATATGVHSQVQLVQSGIEMRKP | 5944 | MGWSCIILFLVATATGV |
| LALA | | GASVKVSCRASGDTFTNCGFGWVRQAPGQGLEW | | HSSSELTQDPTVSVALG |
| PGhL | | MGWISAYNGNTNYAQKFQGRVTMTTDTSTSTAY | | QTVRITCQGDSLRSYYA |
| 2 | | MELRSLRSDDTAVYYCAREGRTNYFYYYMDVWG | | SWYQQKPGQAPVLVIYG |
| | | QGTTVTVSSASTKGPSVFPLAPSSKSTSGGTAA | | KNNRPSGIPDRESGSSS |
| | | LGCLVKDYFPEPVTVSWNSGALTSGVHTFPAVL | | GNTASLTITGAQAEDEA |
| | | QSSGLYSLSSVVTVPSSSLGTQTYICNVNHKPS | | DYYCNSRDSSGNHWVFG |
| | | NTKVDKKVEPKSCDKTHTCPPCPAPEAAGGPSV | | GGTKLTVLGQPKAAPSV |
| | | FLFPPKPKDTLMISRTPEVTCVVVDVSHEDPEV | | TLFPPSSEELQANKATL |
| | | KFNWYVDGVEVHNAKTKPREEQYNSTYRVVSVL | | VCLISDFYPGAVTVAWK |
| | | TVLHQDWLNGKEYKCKVSNKALGAPIEKTISKA | | ADSSPVKAGVETTTPSK |
| | | KGQPREPQVYTLPPSREEMTKNQVSLTCLVKGF | | QSNNKYAASSYLSLTPE |
| | | YPSDIAVEWESNGQPENNYKTTPPVLDSDGSFF | | QWKSHRSYSCQVTHEGS |
| | | LYSKLTVDKSRWQQGNVFSCSVMHEALHNHYTQ | | TVEKTVAPTECS |
| | | KSLSLSPGK | | |
| A142- | 5757 | MGWSCIILFLVATATGVHSEVQLVESGGGLVQP | 5945 | MGWSCIILFLVATATGV |
| LALA | | GRSLRLSCAASGFTEDDYAMHWVRQAPGKGLEW | | HSSSELTQDPAVSVALG |
| PGhL | | VSGISKNSGSIGYADSVKGRFTISRDNAKKSLY | | QTVRITCQGDSLRSYYA |
| 2 | | LQMNSLRVEDTALYYCAKDITASGDYYYMDVWG | | SWYQQKPGQAPVLVIYG |
| | | QGTTVTVSSASTKGPSVFPLAPSSKSTSGGTAA | | KNNRPSGIPDRESGSSS |
| | | LGCLVKDYFPEPVTVSWNSGALTSGVHTFPAVL | | GNTASLTITGAQAEDEA |
| | | QSSGLYSLSSVVTVPSSSLGTQTYICNVNHKPS | | DYYCNSRDSSGNHLVFG |
| | | NTKVDKKVEPKSCDKTHTCPPCPAPEAAGGPSV | | GGTKLTVLGQPKAAPSV |
| | | FLFPPKPKDTLMISRTPEVTCVVVDVSHEDPEV | | TLFPPSSEELQANKATL |
| | | KFNWYVDGVEVHNAKTKPREEQYNSTYRVVSVL | | VCLISDFYPGAVTVAWK |
| | | TVLHQDWLNGKEYKCKVSNKALGAPIEKTISKA | | ADSSPVKAGVETTTPSK |
| | | KGQPREPQVYTLPPSREEMTKNQVSLTCLVKGF | | QSNNKYAASSYLSLTPE |
| | | YPSDIAVEWESNGQPENNYKTTPPVLDSDGSFF | | QWKSHRSYSCQVTHEGS |
| | | LYSKLTVDKSRWQQGNVFSCSVMHEALHNHYTQ | | TVEKTVAPTECS |
| | | KSLSLSPGK | | |
| A143- | 5758 | MGWSCIILFLVATATGVHSEVQLVESGGGLVQP | 5946 | MGWSCIILFLVATATGV |
| LALA | | GGSLRLSCAASGFTFSSYSMNWVRQAPGKGLEW | | HSSSELTPDPAVSVALG |
| PGhL | | VSYISSSSSTIYFADSVKGRFTISRDNAKNSLY | | QTVRITCQGDSLRSYYA |
| 2 | | LQMNSLRDEDTAVYYCARDRVYNWNDGAFDIWG | | SWYQQKPGQAPILVIYH |
| | | QGTMVTVSSASTKGPSVFPLAPSSKSTSGGTAA | | KNNRPSGIPDRFSGSSS |
| | | LGCLVKDYFPEPVTVSWNSGALTSGVHTFPAVL | | GNTASLTITGAQAEDEA |
| | | QSSGLYSLSSVVTVPSSSLGTQTYICNVNHKPS | | DYYCNSRDSSGNHVVFG |
| | | NTKVDKKVEPKSCDKTHTCPPCPAPEAAGGPSV | | GGTKLTVLGQPKAAPSV |
| | | FLFPPKPKDTLMISRTPEVTCVVVDVSHEDPEV | | TLFPPSSEELQANKATL |
| | | KFNWYVDGVEVHNAKTKPREEQYNSTYRVVSVL | | VCLISDFYPGAVTVAWK |
| | | TVLHQDWLNGKEYKCKVSNKALGAPIEKTISKA | | ADSSPVKAGVETTTPSK |
| | | KGQPREPQVYTLPPSREEMTKNQVSLTCLVKGF | | QSNNKYAASSYLSLTPE |
| | | YPSDIAVEWESNGQPENNYKTTPPVLDSDGSFF | | QWKSHRSYSCQVTHEGS |
| | | LYSKLTVDKSRWQQGNVFSCSVMHEALHNHYTQ | | TVEKTVAPTECS |
| | | KSLSLSPGK | | |
| A144- | 5759 | MGWSCIILFLVATATGVHSEVQLVESGGGLVQP | 5947 | MGWSCIILFLVATATGV |
| LALA | | GGSLRLSCAASGFTFSSYAMSWVRQAPGKGLEW | | HSSSELTQDPAVSVALG |
| PGhL | | VSAISGSGGSTYYADSVKGRFTISRDNSKNTMY | | QTVRITCQGDSLRSYYA |
| 2 | | LQMNSLRAEDTAVYYCAKDQRYNWNSWYFDLWG | | SWYQQKPGQAPVLVIYG |
| | | RGTLVTVSSASTKGPSVFPLAPSSKSTSGGTAA | | KNNRPSGIPDRFSGSSS |
| | | LGCLVKDYFPEPVTVSWNSGALTSGVHTFPAVL | | GNTASLTITGAQAEDEA |
| | | QSSGLYSLSSVVTVPSSSLGTQTYICNVNHKPS | | DYYCNSRDSSGNHLVFG |
| | | NTKVDKKVEPKSCDKTHTCPPCPAPEAAGGPSV | | GGTKLTVLGQPKAAPSV |
| | | FLFPPKPKDTLMISRTPEVTCVVVDVSHEDPEV | | TLFPPSSEELQANKATL |
| | | KFNWYVDGVEVHNAKTKPREEQYNSTYRVVSVL | | VCLISDFYPGAVTVAWK |
| | | TVLHQDWLNGKEYKCKVSNKALGAPIEKTISKA | | ADSSPVKAGVETTTPSK |
| | | KGQPREPQVYTLPPSREEMTKNQVSLTCLVKGF | | QSNNKYAASSYLSLTPE |
| | | YPSDIAVEWESNGQPENNYKTTPPVLDSDGSFF | | QWKSHRSYSCQVTHEGS |
| | | LYSKLTVDKSRWQQGNVFSCSVMHEALHNHYTQ | | TVEKTVAPTECS |
| | | KSLSLSPGK | | |
| A145- | 5760 | MGWSCIILFLVATATGVHSQVQLVESGGGVVQP | 5948 | MGWSCIILFLVATATGV |
| LALA | | GRSLRLSCAASGFTFSSYGMHWVRQAPGKGLEW | | HSSYELTQPPSVSVSPG |
| PGhL | | VAVIWYDGSNKYYADSVKGRFTISRDNSKNTLY | | QTARITCSGDALPKKYA |
| 2 | | LQMNSLRAEDTAVYYCARDHGGVTTYNWFDPWG | | YWYQQKSGQAPVLVIYE |
| | | QGTLVTVSSASTKGPSVFPLAPSSKSTSGGTAA | | DSKRPSGIPERFSGSSS |
| | | LGCLVKDYFPEPVTVSWNSGALTSGVHTFPAVL | | GTMATLTISGAQVEDEA |
| | | QSSGLYSLSSVVTVPSSSLGTQTYICNVNHKPS | | DYYCYSTDSSGNHRVFG |
| | | NTKVDKKVEPKSCDKTHTCPPCPAPEAAGGPSV | | GGTKLTVLGQPKAAPSV |
| | | FLFPPKPKDTLMISRTPEVTCVVVDVSHEDPEV | | TLFPPSSEELQANKATL |
| | | KFNWYVDGVEVHNAKTKPREEQYNSTYRVVSVL | | VCLISDFYPGAVTVAWK |
| | | TVLHQDWLNGKEYKCKVSNKALGAPIEKTISKA | | ADSSPVKAGVETTTPSK |
| | | KGQPREPQVYTLPPSREEMTKNQVSLTCLVKGF | | QSNNKYAASSYLSLTPE |
| | | YPSDIAVEWESNGQPENNYKTTPPVLDSDGSFF | | QWKSHRSYSCQVTHEGS |
| | | LYSKLTVDKSRWQQGNVFSCSVMHEALHNHYTQ | | TVEKTVAPTECS |
| | | KSLSLSPGK | | |
| A146- | 5761 | MGWSCIILFLVATATGVHSQVQLVQSGAEVKKP | 5949 | MGWSCIILFLVATATGV |
| LALA | | GASVKVSCKASGYTFTGYYMHWVRQAPGQGLEW | | HSQSALTQPPSASGSPG |
| PGhL | | MGWINPNSGGTNYAQKFQGRVTMTRDTSISTAY | | QSVTISCTGTSSDVGGY |
| 2 | | MELSRLRSDDTAVYYCARDRMVRGVLDAFDIWG | | NYVSWYQQHPGKAPKLM |
| | | QGTMVTVSSASTKGPSVFPLAPSSKSTSGGTAA | | IYEVSKRPSGVPDRFSG |
| | | LGCLVKDYFPEPVTVSWNSGALTSGVHTFPAVL | | SKSGNTASLTVSGLQAE |
| | | QSSGLYSLSSVVTVPSSSLGTQTYICNVNHKPS | | DEADYYCSSYAGSNNVV |
| | | NTKVDKKVEPKSCDKTHTCPPCPAPEAAGGPSV | | FGGGTKLTVLGQPKAAP |
| | | FLFPPKPKDTLMISRTPEVTCVVVDVSHEDPEV | | SVTLFPPSSEELQANKA |
| | | KFNWYVDGVEVHNAKTKPREEQYNSTYRVVSVL | | TLVCLISDFYPGAVTVA |
| | | TVLHQDWLNGKEYKCKVSNKALGAPIEKTISKA | | WKADSSPVKAGVETTTP |
| | | KGQPREPQVYTLPPSREEMTKNQVSLTCLVKGF | | SKQSNNKYAASSYLSLT |
| | | YPSDIAVEWESNGQPENNYKTTPPVLDSDGSFF | | PEQWKSHRSYSCQVTHE |
| | | LYSKLTVDKSRWQQGNVFSCSVMHEALHNHYTQ | | GSTVEKTVAPTECS |
| | | KSLSLSPGK | | |
| A147- | 5762 | MGWSCIILFLVATATGVHSEVQLVESGGGLVQP | 5950 | MGWSCIILFLVATATGV |
| LALA | | GGSLRLSCAASGFTFSSYSMNWVRQAPGKGLEW | | HSQSALTQPPSASGSPG |
| PGhL | | VSYISSSSSTIYYADSVKGRFTISRDNAKNSLY | | QSVTISCTGTSSDVGGY |
| 2 | | LQMNSLRDEDTAVYYCVRGYSSGWYNWYFDLWG | | NYVSWYQQHPGKAPKLM |
| | | RGTLVTVSSASTKGPSVFPLAPSSKSTSGGTAA | | IYEVSKRPSGVPDRFSG |
| | | LGCLVKDYFPEPVTVSWNSGALTSGVHTFPAVL | | SKSGNTASLTVSGLQAE |
| | | QSSGLYSLSSVVTVPSSSLGTQTYICNVNHKPS | | DEADYYCSSYAGSNNLV |
| | | NTKVDKKVEPKSCDKTHTCPPCPAPEAAGGPSV | | FGGGTKLTVLGQPKAAP |
| | | FLFPPKPKDTLMISRTPEVTCVVVDVSHEDPEV | | SVTLFPPSSEELQANKA |
| | | KENWYVDGVEVHNAKTKPREEQYNSTYRVVSVL | | TLVCLISDFYPGAVTVA |
| | | TVLHQDWLNGKEYKCKVSNKALGAPIEKTISKA | | WKADSSPVKAGVETTTP |
| | | KGQPREPQVYTLPPSREEMTKNQVSLTCLVKGF | | SKQSNNKYAASSYLSLT |
| | | YPSDIAVEWESNGQPENNYKTTPPVLDSDGSFF | | PEQWKSHRSYSCQVTHE |
| | | LYSKLTVDKSRWQQGNVFSCSVMHEALHNHYTQ | | GSTVEKTVAPTECS |
| | | KSLSLSPGK | | |
| A148- | 5763 | MGWSCIILFLVATATGVHSQVQLVESGGGLVKP | 5951 | MGWSCIILFLVATATGV |
| LALA | | GGSLRLSCAASGFTFSDYYMSWIRQAPGKGLEW | | HSQTVVTQEPSLTVSPG |
| PGhL | | VSYISSSGSTIYYADSVKGRFTISRDNAKNSLY | | GTVTLTCASSTGAVTSG |
| 2 | | LQMNSLRAEDTAVYYCARKVPGIAAAGAFDYWG | | YYPNWFQQKPGQAPRAL |
| | | QGTLVTVSSASTKGPSVFPLAPSSKSTSGGTAA | | IYSTSNKHSWTPARFSG |
| | | LGCLVKDYFPEPVTVSWNSGALTSGVHTFPAVL | | SLLGGKAALTLSGVQPE |
| | | QSSGLYSLSSVVTVPSSSLGTQTYICNVNHKPS | | DEAEYYCLLYYGGAQLV |
| | | NTKVDKKVEPKSCDKTHTCPPCPAPEAAGGPSV | | FGGGTKLTVLGQPKAAP |
| | | FLFPPKPKDTLMISRTPEVTCVVVDVSHEDPEV | | SVTLFPPSSEELQANKA |
| | | KFNWYVDGVEVHNAKTKPREEQYNSTYRVVSVL | | TLVCLISDFYPGAVTVA |
| | | TVLHQDWLNGKEYKCKVSNKALGAPIEKTISKA | | WKADSSPVKAGVETTTP |
| | | KGQPREPQVYTLPPSREEMTKNQVSLTCLVKGF | | SKQSNNKYAASSYLSLT |
| | | YPSDIAVEWESNGQPENNYKTTPPVLDSDGSFF | | PEQWKSHRSYSCQVTHE |
| | | LYSKLTVDKSRWQQGNVFSCSVMHEALHNHYTQ | | GSTVEKTVAPTECS |
| | | KSLSLSPGK | | |
| A149- | 5764 | MGWSCIILFLVATATGVHSQVQLVQSGSELKKP | 5952 | MGWSCIILFLVATATGV |
| LALA | | GASVKVSCKASGYTFNSYAMNWVRQAPGLGLEW | | HSQSVLTQPPSVSGAPG |
| PGhL | | MGWINTNTGNPTYAQGFSGRFVFSLDTSVNTAY | | QRVTISCTGSNSNIGAG |
| 2 | | LQISSLQAEDTAVYFCARGGYGYNFWIRFDPWG | | YDIHWYQQLPVTAPKLL |
| | | QGTLVTVSSASTKGPSVFPLAPSSKSTSGGTAA | | IYGNSNRPSGVPDRFSG |
| | | LGCLVKDYFPEPVTVSWNSGALTSGVHTFPAVL | | SKSGSSASLAITGLQAE |
| | | QSSGLYSLSSVVTVPSSSLGTQTYICNVNHKPS | | DEADYYCQSYDNSLSGS |
| | | NTKVDKKVEPKSCDKTHTCPPCPAPEAAGGPSV | | VFGGGTKLTVLGQPKAA |
| | | FLFPPKPKDTLMISRTPEVTCVVVDVSHEDPEV | | PSVTLFPPSSEELQANK |
| | | KFNWYVDGVEVHNAKTKPREEQYNSTYRVVSVL | | ATLVCLISDFYPGAVTV |
| | | TVLHQDWLNGKEYKCKVSNKALGAPIEKTISKA | | AWKADSSPVKAGVETTT |
| | | KGQPREPQVYTLPPSREEMTKNQVSLTCLVKGF | | PSKQSNNKYAASSYLSL |
| | | YPSDIAVEWESNGQPENNYKTTPPVLDSDGSFF | | TPEQWKSHRSYSCQVTH |
| | | LYSKLTVDKSRWQQGNVFSCSVMHEALHNHYTQ | | EGSTVEKTVAPTECS |
| | | KSLSLSPGK | | |
| A150- | 5765 | MGWSCIILFLVATATGVHSQLQLQESGPGLVKP | 5953 | MGWSCIILFLVATATGV |
| LALA | | SETLSLTCTVSGGSISRSSYYWGWIRQPPGRGL | | HSSYELTQPSSVSVSPG |
| PGhL | | EWIGSIYYSGSTYYNPSLKSRVTISVDTSKNQF | | QTARITCSGDVLAKKFA |
| 2 | | SLKLSSVTAADTGVYYCASYWNFDYWGRGTLVT | | RWFQQKPGQAPLLVIYK |
| | | VSSASTKGPSVFPLAPSSKSTSGGTAALGCLVK | | DSERPSGIPERFSGSNS |
| | | DYFPEPVTVSWNSGALTSGVHTFPAVLQSSGLY | | GTTVTLTISGAQVEDEA |
| | | SLSSVVTVPSSSLGTQTYICNVNHKPSNTKVDK | | DYYCYSAADNNLVFGGG |
| | | KVEPKSCDKTHTCPPCPAPEAAGGPSVFLFPPK | | TKLTVLGQPKAAPSVTL |
| | | PKDTLMISRTPEVTCVVVDVSHEDPEVKENWYV | | FPPSSEELQANKATLVC |
| | | DGVEVHNAKTKPREEQYNSTYRVVSVLTVLHQD | | LISDFYPGAVTVAWKAD |
| | | WLNGKEYKCKVSNKALGAPIEKTISKAKGQPRE | | SSPVKAGVETTTPSKQS |
| | | PQVYTLPPSREEMTKNQVSLTCLVKGFYPSDIA | | NNKYAASSYLSLTPEQW |
| | | VEWESNGQPENNYKTTPPVLDSDGSFFLYSKLT | | KSHRSYSCQVTHEGSTV |
| | | VDKSRWQQGNVFSCSVMHEALHNHYTQKSLSLS | | EKTVAPTECS |
| | | PGK | | |
| A151- | 5766 | MGWSCIILFLVATATGVHSQVQLQESGPGLVKP | 5954 | MGWSCIILFLVATATGV |
| LALA | | SGTLSLTCAVSGGSISSSFWLSWVRQPPGKGLE | | HSSYELTQPPSVSVSPG |
| PGhL | | WIGEIYHSGSTNYNPSLKSRVTISVDKSKNQFS | | QTARITCSGDALPKKYA |
| 2 | | LKLTSVTAADTAVYSCARVLGYSYGYRRWEDPW | | YWYQQKSGQAPVLVIYE |
| | | GQGTLVTVSSASTKGPSVFPLAPSSKSTSGGTA | | DSKRPSGIPERFSGSSS |
| | | ALGCLVKDYFPEPVTVSWNSGALTSGVHTFPAV | | GTMATLTISGAQVEDEA |
| | | LQSSGLYSLSSVVTVPSSSLGTQTYICNVNHKP | | DYYCYSTDSSGNHRVFG |
| | | SNTKVDKKVEPKSCDKTHTCPPCPAPEAAGGPS | | GGTKLTVLGQPKAAPSV |
| | | VFLFPPKPKDTLMISRTPEVTCVVVDVSHEDPE | | TLFPPSSEELQANKATL |
| | | VKFNWYVDGVEVHNAKTKPREEQYNSTYRVVSV | | VCLISDFYPGAVTVAWK |
| | | LTVLHQDWLNGKEYKCKVSNKALGAPIEKTISK | | ADSSPVKAGVETTTPSK |
| | | AKGQPREPQVYTLPPSREEMTKNQVSLTCLVKG | | QSNNKYAASSYLSLTPE |
| | | FYPSDIAVEWESNGQPENNYKTTPPVLDSDGSF | | QWKSHRSYSCQVTHEGS |
| | | FLYSKLTVDKSRWQQGNVFSCSVMHEALHNHYT | | TVEKTVAPTECS |
| | | QKSLSLSPGK | | |
| A152- | 5767 | MGWSCIILFLVATATGVHSEVQLVESGGGLVNP | 5955 | MGWSCIILFLVATATGV |
| LALA | | GGSLRLSCAASGFTFSNAWMSWVRQGPGRGLEW | | HSSYVLTQPPSVSVAPG |
| PGhL | | VGRIKSKSDGETIDYAAPVKGRFSFSRDDAENT | | TTARITCGGNNFGSKSV |
| 2 | | LYLEMNSLKTEDTAVYYCTTEGSFNFYYFMDVW | | HWYQQKPGQAPVLVIYY |
| | | GQGTMVTVSSASTKGPSVFPLAPSSKSTSGGTA | | DSDRPSGIPERFSGSNS |
| | | ALGCLVKDYFPEPVTVSWNSGALTSGVHTFPAV | | GNTATLTISRVEAGDEA |
| | | LQSSGLYSLSSVVTVPSSSLGTQTYICNVNHKP | | DYYCQVWDSTSDHYVFG |
| | | SNTKVDKKVEPKSCDKTHTCPPCPAPEAAGGPS | | TGTKVTVLGQPKAAPSV |
| | | VFLFPPKPKDTLMISRTPEVTCVVVDVSHEDPE | | TLFPPSSEELQANKATL |
| | | VKFNWYVDGVEVHNAKTKPREEQYNSTYRVVSV | | VCLISDFYPGAVTVAWK |
| | | LTVLHQDWLNGKEYKCKVSNKALGAPIEKTISK | | ADSSPVKAGVETTTPSK |
| | | AKGQPREPQVYTLPPSREEMTKNQVSLTCLVKG | | QSNNKYAASSYLSLTPE |
| | | FYPSDIAVEWESNGQPENNYKTTPPVLDSDGSF | | QWKSHRSYSCQVTHEGS |
| | | FLYSKLTVDKSRWQQGNVFSCSVMHEALHNHYT | | TVEKTVAPTECS |
| | | QKSLSLSPGK | | |
| A153- | 5768 | MGWSCIILFLVATATGVHSQVQLQESGPGLVKP | 5956 | MGWSCIILFLVATATGV |
| LALA | | SETLSLTCTVSGGSISSYYWSWIRQPPGKGLEW | | HSQSALTQPASVSGSPG |
| PGhL | | IGHIYYSGSTNYNPSLKSRVTISVDTSKNQFSL | | QSITISCTGTSSDVGAY |
| 2 | | KLSSVTAADTAVYYCARDPFYYDFSDYYYMDVW | | NYVSWYQQHPGKAPKLM |
| | | GQGTTVTVSSASTKGPSVFPLAPSSKSTSGGTA | | IYAVSKRPSGVSYRFSG |
| | | ALGCLVKDYFPEPVTVSWNSGALTSGVHTFPAV | | SKSGNTASLTISGLQAE |
| | | LQSSGLYSLSSVVTVPSSSLGTQTYICNVNHKP | | DEADYYCCSYAGTISWV |
| | | SNTKVDKKVEPKSCDKTHTCPPCPAPEAAGGPS | | FGGGTKLTVLGQPKAAP |
| | | VFLFPPKPKDTLMISRTPEVTCVVVDVSHEDPE | | SVTLFPPSSEELQANKA |
| | | VKFNWYVDGVEVHNAKTKPREEQYNSTYRVVSV | | TLVCLISDFYPGAVTVA |
| | | LTVLHQDWLNGKEYKCKVSNKALGAPIEKTISK | | WKADSSPVKAGVETTTP |
| | | AKGQPREPQVYTLPPSREEMTKNQVSLTCLVKG | | SKQSNNKYAASSYLSLT |
| | | FYPSDIAVEWESNGQPENNYKTTPPVLDSDGSF | | PEQWKSHRSYSCQVTHE |
| | | FLYSKLTVDKSRWQQGNVFSCSVMHEALHNHYT | | GSTVEKTVAPTECS |
| | | QKSLSLSPGK | | |
| A154- | 5769 | MGWSCIILFLVATATGVHSEVQLVESGGGLVQP | 5957 | MGWSCIILFLVATATGV |
| LALA | | GGSLRLSCAASGFTFSSYAMSWVRQAPGKGLEW | | HSQSALTQPASVSGSPG |
| PGhL | | VSAISGSGGSTYYADSVKGRFTISRDNSKNTLY | | QSITISCTGTSSDVGGY |
| 2 | | LQMNSLRAEDTAVYYCAKNEARDYYGSGSFDYW | | NYVSWYQQHPGKAPKLM |
| | | GQGTLVTVSSASTKGPSVFPLAPSSKSTSGGTA | | IYDVSKRPSGVSNRFSG |
| | | ALGCLVKDYFPEPVTVSWNSGALTSGVHTFPAV | | SKSGNTASLTISGLQAE |
| | | LQSSGLYSLSSVVTVPSSSLGTQTYICNVNHKP | | DEADYYCCSYAGSSTYV |
| | | SNTKVDKKVEPKSCDKTHTCPPCPAPEAAGGPS | | FGTGTKVTVLGQPKAAP |
| | | VFLFPPKPKDTLMISRTPEVTCVVVDVSHEDPE | | SVTLFPPSSEELQANKA |
| | | VKFNWYVDGVEVHNAKTKPREEQYNSTYRVVSV | | TLVCLISDFYPGAVTVA |
| | | LTVLHQDWLNGKEYKCKVSNKALGAPIEKTISK | | WKADSSPVKAGVETTTP |
| | | AKGQPREPQVYTLPPSREEMTKNQVSLTCLVKG | | SKQSNNKYAASSYLSLT |
| | | FYPSDIAVEWESNGQPENNYKTTPPVLDSDGSF | | PEQWKSHRSYSCQVTHE |
| | | FLYSKLTVDKSRWQQGNVFSCSVMHEALHNHYT | | GSTVEKTVAPTECS |
| | | QKSLSLSPGK | | |
| A155- | 5770 | MGWSCIILFLVATATGVHSEVQLVESGGGVVRP | 5958 | MGWSCIILFLVATATGV |
| LALA | | GGSLRLSCAASGFTFGDFGMSWVRQAPGKGLEW | | HSQSALTQPPSASGSPG |
| PGhL | | VSGINWNGGSTGYADSVKGRFTISRDNAKNSLY | | QSVTISCTGTSSDVGGY |
| 2 | | LQMNSLRAEDTALYYCASLVGATDYYFYYMDVW | | NYVSWYQQHPGKAPKLM |
| | | GQGTTVTVSSASTKGPSVFPLAPSSKSTSGGTA | | IYEVNKRPSGVPDRLSG |
| | | ALGCLVKDYFPEPVTVSWNSGALTSGVHTFPAV | | SKSGNTASLTVSGLQAE |
| | | LQSSGLYSLSSVVTVPSSSLGTQTYICNVNHKP | | DEADYYCSSYAGSNNWV |
| | | SNTKVDKKVEPKSCDKTHTCPPCPAPEAAGGPS | | FGGGTKLTVLGQPKAAP |
| | | VFLFPPKPKDTLMISRTPEVTCVVVDVSHEDPE | | SVTLFPPSSEELQANKA |
| | | VKFNWYVDGVEVHNAKTKPREEQYNSTYRVVSV | | TLVCLISDFYPGAVTVA |
| | | LTVLHQDWLNGKEYKCKVSNKALGAPIEKTISK | | WKADSSPVKAGVETTTP |
| | | AKGQPREPQVYTLPPSREEMTKNQVSLTCLVKG | | SKQSNNKYAASSYLSLT |
| | | FYPSDIAVEWESNGQPENNYKTTPPVLDSDGSF | | PEQWKSHRSYSCQVTHE |
| | | FLYSKLTVDKSRWQQGNVFSCSVMHEALHNHYT | | GSTVEKTVAPTECS |
| | | QKSLSLSPGK | | |
| A156- | 5771 | MGWSCIILFLVATATGVHSQVQLQQSGPGLVKP | 5959 | MGWSCIILFLVATATGV |
| LALA | | SQTLSLTCAISGDSVSSNSAAWNWIRQSPSRGL | | HSQSALTQPASVSGSPG |
| PGhL | | EWLGRTYYRSKWYNDYAVSVKSRITINPDTSKN | | QSITISCTGTSSDVGGY |
| 2 | | QFSLQLNSVTPEDTAVYYCARKWELLDAFDIWG | | NYVSWYQQHPGKAPKLM |
| | | QGTMVTVSSASTKGPSVFPLAPSSKSTSGGTAA | | IYDVSKRPSGVSNRFSG |
| | | LGCLVKDYFPEPVTVSWNSGALTSGVHTFPAVL | | SKSGNTASLTISGLQAE |
| | | QSSGLYSLSSVVTVPSSSLGTQTYICNVNHKPS | | DEADYYCCSYAGSSTWV |
| | | NTKVDKKVEPKSCDKTHTCPPCPAPEAAGGPSV | | FGGGTKLTVLGQPKAAP |
| | | FLFPPKPKDTLMISRTPEVTCVVVDVSHEDPEV | | SVTLFPPSSEELQANKA |
| | | KFNWYVDGVEVHNAKTKPREEQYNSTYRVVSVL | | TLVCLISDFYPGAVTVA |
| | | TVLHQDWLNGKEYKCKVSNKALGAPIEKTISKA | | WKADSSPVKAGVETTTP |
| | | KGQPREPQVYTLPPSREEMTKNQVSLTCLVKGF | | SKQSNNKYAASSYLSLT |
| | | YPSDIAVEWESNGQPENNYKTTPPVLDSDGSFF | | PEQWKSHRSYSCQVTHE |
| | | LYSKLTVDKSRWQQGNVFSCSVMHEALHNHYTQ | | GSTVEKTVAPTECS |
| | | KSLSLSPGK | | |
| A157- | 5772 | MGWSCIILFLVATATGVHSEVQLVESGGGLVQP | 5960 | MGWSCIILFLVATATGV |
| LALA | | GGSLRLSCAASGFTFSSYSMNWVRQAPGKGLEW | | HSQSVLTQPPSVSGAPG |
| PGhL | | VSYISSSSSTIYYADSVKGRFTISRDNAKNSLY | | QRVTISCTGSSSNIGAG |
| 2 | | LQMNSLRDEDTAVYYCAREERDDYSNYGYFQHW | | YDVHWYQQLPGTAPKLL |
| | | GQGTLVTVSSASTKGPSVFPLAPSSKSTSGGTA | | IYGNSNRPSGVPDRFSG |
| | | ALGCLVKDYFPEPVTVSWNSGALTSGVHTFPAV | | SKSGTSASLAITGLQAE |
| | | LQSSGLYSLSSVVTVPSSSLGTQTYICNVNHKP | | DEADYYCQSYDSSLSGW |
| | | SNTKVDKKVEPKSCDKTHTCPPCPAPEAAGGPS | | VFGGGTKLTVLGQPKAA |
| | | VFLFPPKPKDTLMISRTPEVTCVVVDVSHEDPE | | PSVTLFPPSSEELQANK |
| | | VKFNWYVDGVEVHNAKTKPREEQYNSTYRVVSV | | ATLVCLISDFYPGAVTV |
| | | LTVLHQDWLNGKEYKCKVSNKALGAPIEKTISK | | AWKADSSPVKAGVETTT |
| | | AKGQPREPQVYTLPPSREEMTKNQVSLTCLVKG | | PSKQSNNKYAASSYLSL |
| | | FYPSDIAVEWESNGQPENNYKTTPPVLDSDGSF | | TPEQWKSHRSYSCQVTH |
| | | FLYSKLTVDKSRWQQGNVFSCSVMHEALHNHYT | | EGSTVEKTVAPTECS |
| | | QKSLSLSPGK | | |
| A158- | 5773 | MGWSCIILFLVATATGVHSQVQLQQSGPGLVKP | 5961 | MGWSCIILFLVATATGV |
| LALA | | SQTLSLTCAISGDSVSSNSATWNWIRQSPSRGL | | HSQSALTQPPSVSGSPG |
| PGhL | | EWLGRSYYMSKWYNDYAVSVKSRITINPDTSKN | | QSVTISCTGTSSDVGSY |
| 2 | | QFSLQLNSVTPEDTAVYYCARGDWNYGVLDSWG | | NRVSWYQQPPGTAPKLM |
| | | QGTLVTVSSASTKGPSVFPLAPSSKSTSGGTAA | | IYDVSNRPSGVPDRFSG |
| | | LGCLVKDYFPEPVTVSWNSGALTSGVHTFPAVL | | SKSGNTASLTISGLQAE |
| | | QSSGLYSLSSVVTVPSSSLGTQTYICNVNHKPS | | DEADYYCSSYTSSSTYV |
| | | NTKVDKKVEPKSCDKTHTCPPCPAPEAAGGPSV | | VFGGGTKLTVLGQPKAA |
| | | FLFPPKPKDTLMISRTPEVTCVVVDVSHEDPEV | | PSVTLFPPSSEELQANK |
| | | KENWYVDGVEVHNAKTKPREEQYNSTYRVVSVL | | ATLVCLISDFYPGAVTV |
| | | TVLHQDWLNGKEYKCKVSNKALGAPIEKTISKA | | AWKADSSPVKAGVETTT |
| | | KGQPREPQVYTLPPSREEMTKNQVSLTCLVKGF | | PSKQSNNKYAASSYLSL |
| | | YPSDIAVEWESNGQPENNYKTTPPVLDSDGSFF | | TPEQWKSHRSYSCQVTH |
| | | LYSKLTVDKSRWQQGNVFSCSVMHEALHNHYTQ | | EGSTVEKTVAPTECS |
| | | KSLSLSPGK | | |
| A159- | 5774 | MGWSCIILFLVATATGVHSQVQLVQSGAEVKKP | 5962 | MGWSCIILFLVATATGV |
| LALA | | RASVKVSCEASGYTFTTYGISWVRQAPGQGLEW | | HSQSVLTQPPSVSATPG |
| PGhL | | MGWISAYNGNTKYTQKLQGRVAMTTDTSTSTAY | | QRVTISCTGSDSNIGAG |
| 2 | | MEVRSLRSDDTAVYYCARSGYNWNYDYYFMDVW | | YDVHWYQQLPGAVPRLL |
| | | GQGTTVTVSSASTKGPSVFPLAPSSKSTSGGTA | | IHDNIIRPSGVPDRFSG |
| | | ALGCLVKDYFPEPVTVSWNSGALTSGVHTFPAV | | SKSDTSASLAISGLHAE |
| | | LQSSGLYSLSSVVTVPSSSLGTQTYICNVNHKP | | DEADYYCQSYDISLSGS |
| | | SNTKVDKKVEPKSCDKTHTCPPCPAPEAAGGPS | | VVFGGGTKLTVLGQPKA |
| | | VFLFPPKPKDTLMISRTPEVTCVVVDVSHEDPE | | APSVTLFPPSSEELQAN |
| | | VKFNWYVDGVEVHNAKTKPREEQYNSTYRVVSV | | KATLVCLISDFYPGAVT |
| | | LTVLHQDWLNGKEYKCKVSNKALGAPIEKTISK | | VAWKADSSPVKAGVETT |
| | | AKGQPREPQVYTLPPSREEMTKNQVSLTCLVKG | | TPSKQSNNKYAASSYLS |
| | | FYPSDIAVEWESNGQPENNYKTTPPVLDSDGSF | | LTPEQWKSHRSYSCQVT |
| | | FLYSKLTVDKSRWQQGNVFSCSVMHEALHNHYT | | HEGSTVEKTVAPTECS |
| | | QKSLSLSPGK | | |
| A160- | 5775 | MGWSCIILFLVATATGVHSEVQLVESGGGVVRP | 5963 | MGWSCIILFLVATATGV |
| LALA | | GGSLRLSCAASGFTFDDYGMSWVRQAPGKGLEW | | HSSYELTQPPSVSVSPG |
| PGhL | | VSGINWNGGSTGYADSVKGRFTISRDNAKNSLY | | QTASITCSGDKLGDKYA |
| 2 | | LQMNSLRAEDTALYYCARDGCSSTSCYGNWFDP | | CWYQQKPGQSPVLVIYQ |
| | | WGQGTLVTVSSASTKGPSVFPLAPSSKSTSGGT | | DSKRPSGIPERFSGSNS |
| | | AALGCLVKDYFPEPVTVSWNSGALTSGVHTFPA | | GNTATLTISGTQAMDEA |
| | | VLQSSGLYSLSSVVTVPSSSLGTQTYICNVNHK | | DYYCQAWDSSTAVFGGG |
| | | PSNTKVDKKVEPKSCDKTHTCPPCPAPEAAGGP | | TKLTVLGQPKAAPSVTL |
| | | SVFLFPPKPKDTLMISRTPEVTCVVVDVSHEDP | | FPPSSEELQANKATLVC |
| | | EVKENWYVDGVEVHNAKTKPREEQYNSTYRVVS | | LISDFYPGAVTVAWKAD |
| | | VLTVLHQDWLNGKEYKCKVSNKALGAPIEKTIS | | SSPVKAGVETTTPSKQS |
| | | KAKGQPREPQVYTLPPSREEMTKNQVSLTCLVK | | NNKYAASSYLSLTPEQW |
| | | GFYPSDIAVEWESNGQPENNYKTTPPVLDSDGS | | KSHRSYSCQVTHEGSTV |
| | | FFLYSKLTVDKSRWQQGNVFSCSVMHEALHNHY | | EKTVAPTECS |
| | | TQKSLSLSPGK | | |
| A161- | 5776 | MGWSCIILFLVATATGVHSQVQLQQSGPGLVKP | 5964 | MGWSCIILFLVATATGV |
| LALA | | SQTLSLTCAISGDSVSSNSAAWNWIRQSPSRGL | | HSSYELTQPPSVSVSPG |
| PGhL | | EWLGRTYYRSKWYSDYAVSVKSRITINPDTSKN | | QTARITCSGDGLSKKYA |
| 2 | | QFSLQLNSVTPEDTAVYYCARVDFGIVGAIDYW | | YWYQQKSGQAPVLVIYE |
| | | GQGTLVTVSSASTKGPSVFPLAPSSKSTSGGTA | | DSKRPSGIPERFSGSSS |
| | | ALGCLVKDYFPEPVTVSWNSGALTSGVHTFPAV | | GTMATLTVSGAQVEDEA |
| | | LQSSGLYSLSSVVTVPSSSLGTQTYICNVNHKP | | DYYCYSTDSSGKIFGGG |
| | | SNTKVDKKVEPKSCDKTHTCPPCPAPEAAGGPS | | TKLTVLGQPKAAPSVTL |
| | | VFLFPPKPKDTLMISRTPEVTCVVVDVSHEDPE | | FPPSSEELQANKATLVC |
| | | VKFNWYVDGVEVHNAKTKPREEQYNSTYRVVSV | | LISDFYPGAVTVAWKAD |
| | | LTVLHQDWLNGKEYKCKVSNKALGAPIEKTISK | | SSPVKAGVETTTPSKQS |
| | | AKGQPREPQVYTLPPSREEMTKNQVSLTCLVKG | | NNKYAASSYLSLTPEQW |
| | | FYPSDIAVEWESNGQPENNYKTTPPVLDSDGSF | | KSHRSYSCQVTHEGSTV |
| | | FLYSKLTVDKSRWQQGNVFSCSVMHEALHNHYT | | EKTVAPTECS |
| | | QKSLSLSPGK | | |
| A162- | 5777 | MGWSCIILFLVATATGVHSEVQLVESGGGLVKP | 5965 | MGWSCIILFLVATATGV |
| LALA | | GGSLRLSCAASAFTFSNYNMNWVRQAPGKGLEW | | HSSSELTQDPAVSVALG |
| PGhL | | VSSISSSTSYIYYADSVKGRFTVSRDNAKNSLY | | QTVRITCQGDSLRSYYA |
| 2 | | LQMNSLRAEDTAVYYCARDRDDFWSGYSPYFDY | | SWYQQKPGQAPVLVIYG |
| | | WGQGTLVTVSSASTKGPSVFPLAPSSKSTSGGT | | KNNRPSGIPDRFSGSSS |
| | | AALGCLVKDYFPEPVTVSWNSGALTSGVHTFPA | | GNTASLTITGAQAEDEA |
| | | VLQSSGLYSLSSVVTVPSSSLGTQTYICNVNHK | | DYYCNSRDSSGNHWVFG |
| | | PSNTKVDKKVEPKSCDKTHTCPPCPAPEAAGGP | | GGTKLTVLGQPKAAPSV |
| | | SVFLFPPKPKDTLMISRTPEVTCVVVDVSHEDP | | TLFPPSSEELQANKATL |
| | | EVKFNWYVDGVEVHNAKTKPREEQYNSTYRVVS | | VCLISDFYPGAVTVAWK |
| | | VLTVLHQDWLNGKEYKCKVSNKALGAPIEKTIS | | ADSSPVKAGVETTTPSK |
| | | KAKGQPREPQVYTLPPSREEMTKNQVSLTCLVK | | QSNNKYAASSYLSLTPE |
| | | GFYPSDIAVEWESNGQPENNYKTTPPVLDSDGS | | QWKSHRSYSCQVTHEGS |
| | | FFLYSKLTVDKSRWQQGNVFSCSVMHEALHNHY | | TVEKTVAPTECS |
| | | TQKSLSLSPGK | | |
| A163- | 5778 | MGWSCIILFLVATATGVHSEVQLVESGGGLVKP | 5966 | MGWSCIILFLVATATGV |
| LALA | | GGSLRLSCAASGFTFSSYSMNWVRQAPGKGLEW | | HSSSELTQDPAVSVALG |
| PGhL | | VSSISSSSSYIYYADSVKGRFTISRDNAKNSLY | | QTVRITCQGDSLRSYYA |
| 2 | | LQMNSLRAEDTAVYYCAREKYDILTGYSPYFDY | | SWYQQKPGQAPVLVIYG |
| | | WGQGTLVTVSSASTKGPSVFPLAPSSKSTSGGT | | KNNRPSGIPDRESGSSS |
| | | AALGCLVKDYFPEPVTVSWNSGALTSGVHTFPA | | GNTASLTITGAQAEDEA |
| | | VLQSSGLYSLSSVVTVPSSSLGTQTYICNVNHK | | DYYCNSRDSSGNHWVFG |
| | | PSNTKVDKKVEPKSCDKTHTCPPCPAPEAAGGP | | GGTKLTVLGQPKAAPSV |
| | | SVFLFPPKPKDTLMISRTPEVTCVVVDVSHEDP | | TLFPPSSEELQANKATL |
| | | EVKFNWYVDGVEVHNAKTKPREEQYNSTYRVVS | | VCLISDFYPGAVTVAWK |
| | | VLTVLHQDWLNGKEYKCKVSNKALGAPIEKTIS | | ADSSPVKAGVETTTPSK |
| | | KAKGQPREPQVYTLPPSREEMTKNQVSLTCLVK | | QSNNKYAASSYLSLTPE |
| | | GFYPSDIAVEWESNGQPENNYKTTPPVLDSDGS | | QWKSHRSYSCQVTHEGS |
| | | FFLYSKLTVDKSRWQQGNVFSCSVMHEALHNHY | | TVEKTVAPTECS |
| | | TQKSLSLSPGK | | |
| A164- | 5779 | MGWSCIILFLVATATGVHSEVQLVESGGGLVKP | 5967 | MGWSCIILFLVATATGV |
| LALA | | GGSLRLSCAASGFTFSNAWMSWVRQAPGKGLEW | | HSSYELTQPPSVSVSPG |
| PGhL | | VGRIKSKTDGGTTDYAAPVKGRFTISRDDSKNT | | QTARITCSGDALPKKYA |
| 2 | | LYLQMNSLKTEDTAVYYCTTDQVSGSYGDAFDI | | YWYQQKSGQAPVLVIYE |
| | | WGQGTMVTVSSASTKGPSVFPLAPSSKSTSGGT | | DSKRPSGIPERFSGSSS |
| | | AALGCLVKDYFPEPVTVSWNSGALTSGVHTFPA | | GTMATLTISGAQVEDEA |
| | | VLQSSGLYSLSSVVTVPSSSLGTQTYICNVNHK | | DYYCYSTDSSGNHRVFG |
| | | PSNTKVDKKVEPKSCDKTHTCPPCPAPEAAGGP | | GGTKLTVLGQPKAAPSV |
| | | SVFLFPPKPKDTLMISRTPEVTCVVVDVSHEDP | | TLFPPSSEELQANKATL |
| | | EVKFNWYVDGVEVHNAKTKPREEQYNSTYRVVS | | VCLISDFYPGAVTVAWK |
| | | VLTVLHQDWLNGKEYKCKVSNKALGAPIEKTIS | | ADSSPVKAGVETTTPSK |
| | | KAKGQPREPQVYTLPPSREEMTKNQVSLTCLVK | | QSNNKYAASSYLSLTPE |
| | | GFYPSDIAVEWESNGQPENNYKTTPPVLDSDGS | | QWKSHRSYSCQVTHEGS |
| | | FFLYSKLTVDKSRWQQGNVFSCSVMHEALHNHY | | TVEKTVAPTECS |
| | | TQKSLSLSPGK | | |
| A165- | 5780 | MGWSCIILFLVATATGVHSEVQLVESGGGLVQP | 5968 | MGWSCIILFLVATATGV |
| LALA | | GGSLRLSCAASGFTFSSHVMSWVRQAPGKGLEW | | HSSSELTQDPAVSVALG |
| PGhL | | VSVISGSESSTYYADSVKGRFTISSDNSKNTLY | | QTVRITCQGDSLRSYYA |
| 2 | | LQMNSLRAEDTAIYYCAKRAGSGTYYRGYYFDY | | SWYQQKPGQAPVLVIYG |
| | | WGQGTLVTVSSASTKGPSVFPLAPSSKSTSGGT | | KNNRPSGIPDRFSGSSS |
| | | AALGCLVKDYFPEPVTVSWNSGALTSGVHTFPA | | GNTASLTITGAQAEDEA |
| | | VLQSSGLYSLSSVVTVPSSSLGTQTYICNVNHK | | DYYCNSRDSSGNHWVFG |
| | | PSNTKVDKKVEPKSCDKTHTCPPCPAPEAAGGP | | GGTKLTVLGQPKAAPSV |
| | | SVFLFPPKPKDTLMISRTPEVTCVVVDVSHEDP | | TLFPPSSEELQANKATL |
| | | EVKFNWYVDGVEVHNAKTKPREEQYNSTYRVVS | | VCLISDFYPGAVTVAWK |
| | | VLTVLHQDWLNGKEYKCKVSNKALGAPIEKTIS | | ADSSPVKAGVETTTPSK |
| | | KAKGQPREPQVYTLPPSREEMTKNQVSLTCLVK | | QSNNKYAASSYLSLTPE |
| | | GFYPSDIAVEWESNGQPENNYKTTPPVLDSDGS | | QWKSHRSYSCQVTHEGS |
| | | FFLYSKLTVDKSRWQQGNVFSCSVMHEALHNHY | | TVEKTVAPTECS |
| | | TQKSLSLSPGK | | |
| A166- | 5781 | MGWSCIILFLVATATGVHSEVQLVESGGGVVQP | 5969 | MGWSCIILFLVATATGV |
| LALA | | GRSLRLSCAASGFTFSSYGMHWVRQAPGKGLEW | | HSSSELTQDPAVSVALG |
| PGhL | | VTLIWYDGSNTYYAESVKGRFTISRDNSKSTLY | | QTVRITCQGDSLRSYYA |
| 2 | | LHMNSLRAEDSAVYYCAGTYYYDSSGYLNYMDV | | SWYQQKPGQAPVLVIYG |
| | | WGQGTTVTVSSASTKGPSVFPLAPSSKSTSGGT | | KNNRPSGIPDRFSGSSS |
| | | AALGCLVKDYFPEPVTVSWNSGALTSGVHTFPA | | GNTASLTITGAQAEDEA |
| | | VLQSSGLYSLSSVVTVPSSSLGTQTYICNVNHK | | DYYCNSRDSSGNHLVFG |
| | | PSNTKVDKKVEPKSCDKTHTCPPCPAPEAAGGP | | GGTKLTVLGQPKAAPSV |
| | | SVFLFPPKPKDTLMISRTPEVTCVVVDVSHEDP | | TLFPPSSEELQANKATL |
| | | EVKFNWYVDGVEVHNAKTKPREEQYNSTYRVVS | | VCLISDFYPGAVTVAWK |
| | | VLTVLHQDWLNGKEYKCKVSNKALGAPIEKTIS | | ADSSPVKAGVETTTPSK |
| | | KAKGQPREPQVYTLPPSREEMTKNQVSLTCLVK | | QSNNKYAASSYLSLTPE |
| | | GFYPSDIAVEWESNGQPENNYKTTPPVLDSDGS | | QWKSHRSYSCQVTHEGS |
| | | FFLYSKLTVDKSRWQQGNVFSCSVMHEALHNHY | | TVEKTVAPTECS |
| | | TQKSLSLSPGK | | |
| A167- | 5782 | MGWSCIILFLVATATGVHSQLQLQESGPGLVKP | 5970 | MGWSCIILFLVATATGV |
| LALA | | SETLSLTCTVSGGSISSSSYYWGWIRQPPGKGL | | HSSSELTQDPAVSVALG |
| PGhL | | EWIGSIHYSGSTYYNPSLKSRVTTSVDTSKNQF | | QTVRITCQGDSLRSYYA |
| 2 | | SLKLSSVTAADTAVYYCASEGPYFDYWGQGTLV | | SWYQQKPGQAPVLVIYG |
| | | TVSSASTKGPSVFPLAPSSKSTSGGTAALGCLV | | KNNRPSGIPDRFSGSSS |
| | | KDYFPEPVTVSWNSGALTSGVHTFPAVLQSSGL | | GNTASLTITGAQAEDEA |
| | | YSLSSVVTVPSSSLGTQTYICNVNHKPSNTKVD | | DYYCNSRDSSGNHWVFG |
| | | KKVEPKSCDKTHTCPPCPAPEAAGGPSVFLFPP | | GGTKLTVLGQPKAAPSV |
| | | KPKDTLMISRTPEVTCVVVDVSHEDPEVKENWY | | TLFPPSSEELQANKATL |
| | | VDGVEVHNAKTKPREEQYNSTYRVVSVLTVLHQ | | VCLISDFYPGAVTVAWK |
| | | DWLNGKEYKCKVSNKALGAPIEKTISKAKGQPR | | ADSSPVKAGVETTTPSK |
| | | EPQVYTLPPSREEMTKNQVSLTCLVKGFYPSDI | | QSNNKYAASSYLSLTPE |
| | | AVEWESNGQPENNYKTTPPVLDSDGSFFLYSKL | | QWKSHRSYSCQVTHEGS |
| | | TVDKSRWQQGNVFSCSVMHEALHNHYTQKSLSL | | TVEKTVAPTECS |
| | | SPGK | | |
| A168- | 5783 | MGWSCIILFLVATATGVHSQVQLVQSGAEVKKP | 5971 | MGWSCIILFLVATATGV |
| LALA | | GASVKVSCKASGYSFSSYGIGWVRQAPGQGLEW | | HSQSALTQPASVSGSPG |
| PGhL | | MGWISGYNGNTNYAQKFQGRVTMTTDTSTSTAH | | QSITISCTGTSSDVGGY |
| 2 | | MEVKSLRSDDTAAYYCARAYCGGDCYYSNAFDA | | NYVSWYQQHPGKAPKLM |
| | | WGQGTMVTVSSASTKGPSVFPLAPSSKSTSGGT | | IYEVSNRPSGVSNRFSG |
| | | AALGCLVKDYFPEPVTVSWNSGALTSGVHTFPA | | SKSGNTASLTISGLQAE |
| | | VLQSSGLYSLSSVVTVPSSSLGTQTYICNVNHK | | DEADYYCSSYTSSSTVV |
| | | PSNTKVDKKVEPKSCDKTHTCPPCPAPEAAGGP | | FGGGTKLTVLGQPKAAP |
| | | SVFLFPPKPKDTLMISRTPEVTCVVVDVSHEDP | | SVTLFPPSSEELQANKA |
| | | EVKFNWYVDGVEVHNAKTKPREEQYNSTYRVVS | | TLVCLISDFYPGAVTVA |
| | | VLTVLHQDWLNGKEYKCKVSNKALGAPIEKTIS | | WKADSSPVKAGVETTTP |
| | | KAKGQPREPQVYTLPPSREEMTKNQVSLTCLVK | | SKQSNNKYAASSYLSLT |
| | | GFYPSDIAVEWESNGQPENNYKTTPPVLDSDGS | | PEQWKSHRSYSCQVTHE |
| | | FFLYSKLTVDKSRWQQGNVFSCSVMHEALHNHY | | GSTVEKTVAPTECS |
| | | TQKSLSLSPGK | | |
| A169- | 5784 | MGWSCIILFLVATATGVHSEVQLVESGGGLVKP | 5972 | MGWSCIILFLVATATGV |
| LALA | | GGSLRLSCAASGFTFSNAWMSWVRQAPGKGLEW | | HSQSALTQPASVSGSPG |
| PGhL | | VGRIKSKTDGGTTDYAAPVKGRFTISRDDSKNT | | QSITISCTGTSSDVGGY |
| 2 | | LYLQMNSLKTEDTAVYYCTTDRVTIFGLARMDV | | NYVSWYQQHPGKAPKLM |
| | | WGQGTTVTVSSASTKGPSVFPLAPSSKSTSGGT | | IYDVSKRPSGVSNRFSG |
| | | AALGCLVKDYFPEPVTVSWNSGALTSGVHTFPA | | SKSGNTASLTISGLQAE |
| | | VLQSSGLYSLSSVVTVPSSSLGTQTYICNVNHK | | DEADYYCCSYAGSSTWV |
| | | PSNTKVDKKVEPKSCDKTHTCPPCPAPEAAGGP | | FGGGTKLTVLGQPKAAP |
| | | SVFLFPPKPKDTLMISRTPEVTCVVVDVSHEDP | | SVTLFPPSSEELQANKA |
| | | EVKFNWYVDGVEVHNAKTKPREEQYNSTYRVVS | | TLVCLISDFYPGAVTVA |
| | | VLTVLHQDWLNGKEYKCKVSNKALGAPIEKTIS | | WKADSSPVKAGVETTTP |
| | | KAKGQPREPQVYTLPPSREEMTKNQVSLTCLVK | | SKQSNNKYAASSYLSLT |
| | | GFYPSDIAVEWESNGQPENNYKTTPPVLDSDGS | | PEQWKSHRSYSCQVTHE |
| | | FFLYSKLTVDKSRWQQGNVFSCSVMHEALHNHY | | GSTVEKTVAPTECS |
| | | TQKSLSLSPGK | | |
| A170- | 5785 | MGWSCIILFLVATATGVHSEVQLVESGGGLVQP | 5973 | MGWSCIILFLVATATGV |
| LALA | | GGSLRLSCAASGFTFSTYSVKWVRQAPGKGLEW | | HSSSELTQDPAVSVALG |
| PGhL | | VSYISSGSSTIYYADSVKGRFTISRDNAKNSLY | | QTVRITCQGDSLRSYYA |
| 2 | | LQMNSLRDEDTAVYYCARDPTTIFGVVPYYYMD | | TWYQQKPGQAPVLVIYG |
| | | VWGTGTTVTVSSASTKGPSVFPLAPSSKSTSGG | | RNNRPSGIPDRFSGSSS |
| | | TAALGCLVKDYFPEPVTVSWNSGALTSGVHTFP | | GNTASLTITGAQAEDEA |
| | | AVLQSSGLYSLSSVVTVPSSSLGTQTYICNVNH | | DYYCYSRDSSGNHLVFG |
| | | KPSNTKVDKKVEPKSCDKTHTCPPCPAPEAAGG | | GGTQLTVLGQPKAAPSV |
| | | PSVFLFPPKPKDTLMISRTPEVTCVVVDVSHED | | TLFPPSSEELQANKATL |
| | | PEVKFNWYVDGVEVHNAKTKPREEQYNSTYRVV | | VCLISDFYPGAVTVAWK |
| | | SVLTVLHQDWLNGKEYKCKVSNKALGAPIEKTI | | ADSSPVKAGVETTTPSK |
| | | SKAKGQPREPQVYTLPPSREEMTKNQVSLTCLV | | QSNNKYAASSYLSLTPE |
| | | KGFYPSDIAVEWESNGQPENNYKTTPPVLDSDG | | QWKSHRSYSCQVTHEGS |
| | | SFFLYSKLTVDKSRWQQGNVFSCSVMHEALHNH | | TVEKTVAPTECS |
| | | YTQKSLSLSPGK | | |
| A171- | 5786 | MGWSCIILFLVATATGVHSEVQLVESGGGLVKP | 5974 | MGWSCIILFLVATATGV |
| LALA | | GGSLRLSCAASGFTFSNAWMSWVRQAPGKGLEW | | HSSYVLTQPPSVSVAPG |
| PGhL | | VGRIKSKTDGGTTDYAAPVKGRFTISRDDSKNT | | KTARITCGGNNIGSKSV |
| 2 | | LYLQMNSLKTEDTAVYYCTTDRDYYGSGSYYED | | HWYQQKPGQAPVLVIYY |
| | | YWGQGTLVTVSSASTKGPSVFPLAPSSKSTSGG | | DSDRPSGIPERFSGSNS |
| | | TAALGCLVKDYFPEPVTVSWNSGALTSGVHTFP | | GNTATLTISRVEAGDEA |
| | | AVLQSSGLYSLSSVVTVPSSSLGTQTYICNVNH | | DYYCQVWDSSSDHRVFG |
| | | KPSNTKVDKKVEPKSCDKTHTCPPCPAPEAAGG | | GGTKLTVLGQPKAAPSV |
| | | PSVFLFPPKPKDTLMISRTPEVTCVVVDVSHED | | TLFPPSSEELQANKATL |
| | | PEVKFNWYVDGVEVHNAKTKPREEQYNSTYRVV | | VCLISDFYPGAVTVAWK |
| | | SVLTVLHQDWLNGKEYKCKVSNKALGAPIEKTI | | ADSSPVKAGVETTTPSK |
| | | SKAKGQPREPQVYTLPPSREEMTKNQVSLTCLV | | QSNNKYAASSYLSLTPE |
| | | KGFYPSDIAVEWESNGQPENNYKTTPPVLDSDG | | QWKSHRSYSCQVTHEGS |
| | | SFFLYSKLTVDKSRWQQGNVFSCSVMHEALHNH | | TVEKTVAPTECS |
| | | YTQKSLSLSPGK | | |
| A172- | 5787 | MGWSCIILFLVATATGVHSQLQLQESGPGLVKP | 5975 | MGWSCIILFLVATATGV |
| LALA | | SETLSLTCTVSGGSITTRSYYWGWLRQPPGKGL | | HSSSELTQDPAVSVALG |
| PGhL | | EWIGTFYYSGNTYYNPSLQSRVSISVDASKNQF | | QTVRITCQGDSLRSYYA |
| 2 | | SLQLSSVTAADTAVFYCAREDLIGNDYWGQGTL | | SWYQQKPGQAPVLVIYG |
| | | VTVSSASTKGPSVFPLAPSSKSTSGGTAALGCL | | KNNRPSGIPDRFSGSSS |
| | | VKDYFPEPVTVSWNSGALTSGVHTFPAVLQSSG | | GNTASLTITGAQAEDEA |
| | | LYSLSSVVTVPSSSLGTQTYICNVNHKPSNTKV | | DYYCNSRDSSGNHLVFG |
| | | DKKVEPKSCDKTHTCPPCPAPEAAGGPSVFLFP | | GGTKLTVLGQPKAAPSV |
| | | PKPKDTLMISRTPEVTCVVVDVSHEDPEVKENW | | TLFPPSSEELQANKATL |
| | | YVDGVEVHNAKTKPREEQYNSTYRVVSVLTVLH | | VCLISDFYPGAVTVAWK |
| | | QDWLNGKEYKCKVSNKALGAPIEKTISKAKGQP | | ADSSPVKAGVETTTPSK |
| | | REPQVYTLPPSREEMTKNQVSLTCLVKGFYPSD | | QSNNKYAASSYLSLTPE |
| | | IAVEWESNGQPENNYKTTPPVLDSDGSFFLYSK | | QWKSHRSYSCQVTHEGS |
| | | LTVDKSRWQQGNVFSCSVMHEALHNHYTQKSLS | | TVEKTVAPTECS |
| | | LSPGK | | |
| A173- | 5788 | MGWSCIILFLVATATGVHSQVQLQQSGPGLVKP | 5976 | MGWSCIILFLVATATGV |
| LALA | | SQTLSLTCAISGDSVSSNSAAWNWIRQSPSRGL | | HSSSELTQDPAVSVALG |
| PGhL | | EWLGRTYYRSKWYNDYAVSVKSRITINSDTSKN | | QTVRITCQGDSLRSYYA |
| 2 | | QFSLQLNSVTPEDTAVYYCSRDRLIVGASYFDL | | SWYQQKPGQAPVLVFYG |
| | | WGRGTLVTVSSASTKGPSVFPLAPSSKSTSGGT | | KNKRPSGIPDRFSGSSS |
| | | AALGCLVKDYFPEPVTVSWNSGALTSGVHTFPA | | GNTASLTITGAQAEDEA |
| | | VLQSSGLYSLSSVVTVPSSSLGTQTYICNVNHK | | DYYCNSRDSNGNHWVFG |
| | | PSNTKVDKKVEPKSCDKTHTCPPCPAPEAAGGP | | GGTKLTVLGQPKAAPSV |
| | | SVFLFPPKPKDTLMISRTPEVTCVVVDVSHEDP | | TLFPPSSEELQANKATL |
| | | EVKFNWYVDGVEVHNAKTKPREEQYNSTYRVVS | | VCLISDFYPGAVTVAWK |
| | | VLTVLHQDWLNGKEYKCKVSNKALGAPIEKTIS | | ADSSPVKAGVETTTPSK |
| | | KAKGQPREPQVYTLPPSREEMTKNQVSLTCLVK | | QSNNKYAASSYLSLTPE |
| | | GFYPSDIAVEWESNGQPENNYKTTPPVLDSDGS | | QWKSHRSYSCQVTHEGS |
| | | FFLYSKLTVDKSRWQQGNVFSCSVMHEALHNHY | | TVEKTVAPTECS |
| | | TQKSLSLSPGK | | |
| A174- | 5789 | MGWSCIILFLVATATGVHSEVQLVESGGGVVRP | 5977 | MGWSCIILFLVATATGV |
| LALA | | GGSLRLSCAASGFTFDDYGMSWVRQAPGKGLEW | | HSQSALTQPASVSGSPG |
| PGhL | | VSGINWNGGSTGYADSVKGRFTISRDNAKNSLY | | QSITISCTGTSSDVGGY |
| 2 | | LQMNSLRAEDTALYYCAREKAPAHRSSWSWYED | | NYVSWYQQHPGKAPKLM |
| | | LWGRGTLVTVSSASTKGPSVFPLAPSSKSTSGG | | IYDVSKRPSGVSNRFSG |
| | | TAALGCLVKDYFPEPVTVSWNSGALTSGVHTFP | | SKSGNTASLTISGLQAE |
| | | AVLQSSGLYSLSSVVTVPSSSLGTQTYICNVNH | | DEADYYCCSYAGSSWVF |
| | | KPSNTKVDKKVEPKSCDKTHTCPPCPAPEAAGG | | GGGTKLTVLGQPKAAPS |
| | | PSVFLFPPKPKDTLMISRTPEVTCVVVDVSHED | | VTLFPPSSEELQANKAT |
| | | PEVKFNWYVDGVEVHNAKTKPREEQYNSTYRVV | | LVCLISDFYPGAVTVAW |
| | | SVLTVLHQDWLNGKEYKCKVSNKALGAPIEKTI | | KADSSPVKAGVETTTPS |
| | | SKAKGQPREPQVYTLPPSREEMTKNQVSLTCLV | | KQSNNKYAASSYLSLTP |
| | | KGFYPSDIAVEWESNGQPENNYKTTPPVLDSDG | | EQWKSHRSYSCQVTHEG |
| | | SFFLYSKLTVDKSRWQQGNVFSCSVMHEALHNH | | STVEKTVAPTECS |
| | | YTQKSLSLSPGK | | |
| A175- | 5790 | MGWSCIILFLVATATGVHSQVQLVQSGAEVKKP | 5978 | MGWSCIILFLVATATGV |
| LALA | | GASVKVSCKASGYTFTGNYIHWVRQAPGQGLEW | | HSQSVLTQPPSVSGVPG |
| PGhL | | MGWINPTSGVTNYAQKFQGRVTLTRDTSISTAY | | QRVTISCTGSSSNIGAR |
| 2 | | MELSRLRSDDTAVYYCTREGIAAANPGYFYYMD | | YDVHWYQQLPGTAPKLL |
| | | VWGKGTTVTVSSASTKGPSVFPLAPSSKSTSGG | | IYGNSNRPSGVPDRFSG |
| | | TAALGCLVKDYFPEPVTVSWNSGALTSGVHTFP | | SKSGTSASLAITGLQAE |
| | | AVLQSSGLYSLSSVVTVPSSSLGTQTYICNVNH | | DEADYYCQSYDGTLGGW |
| | | KPSNTKVDKKVEPKSCDKTHTCPPCPAPEAAGG | | IFGGGTQLTVLGQPKAA |
| | | PSVFLFPPKPKDTLMISRTPEVTCVVVDVSHED | | PSVTLFPPSSEELQANK |
| | | PEVKFNWYVDGVEVHNAKTKPREEQYNSTYRVV | | ATLVCLISDFYPGAVTV |
| | | SVLTVLHQDWLNGKEYKCKVSNKALGAPIEKTI | | AWKADSSPVKAGVETTT |
| | | SKAKGQPREPQVYTLPPSREEMTKNQVSLTCLV | | PSKQSNNKYAASSYLSL |
| | | KGFYPSDIAVEWESNGQPENNYKTTPPVLDSDG | | TPEQWKSHRSYSCQVTH |
| | | SFFLYSKLTVDKSRWQQGNVFSCSVMHEALHNH | | EGSTVEKTVAPTECS |
| | | YTQKSLSLSPGK | | |
| A176- | 5791 | MGWSCIILFLVATATGVHSQVQLVQSGAEVKKP | 5979 | MGWSCIILFLVATATGV |
| LALA | | GASVKVSCKASGYTFTDYYMHWVRQAPGQGLEW | | HSSSELTQDPAVSVALG |
| PGhL | | MGWINPNSGGTNYAQKFQGRVTMTRDTSISTAY | | QTVRITCQGDSLRSYYA |
| 2 | | MELSRLRSDDTAVYFCARCDMVRGVIDHYYNYM | | SWYQQKPGQAPVLVIYG |
| | | DVWGQGTTVTVSSASTKGPSVFPLAPSSKSTSG | | KNNRPSGIPDRFSGSSS |
| | | GTAALGCLVKDYFPEPVTVSWNSGALTSGVHTF | | GNTASLTITGAQAEDEA |
| | | PAVLQSSGLYSLSSVVTVPSSSLGTQTYICNVN | | DYYCNSRDSSGNHWVFG |
| | | HKPSNTKVDKKVEPKSCDKTHTCPPCPAPEAAG | | GGTKLTVLGQPKAAPSV |
| | | GPSVFLFPPKPKDTLMISRTPEVTCVVVDVSHE | | TLFPPSSEELQANKATL |
| | | DPEVKFNWYVDGVEVHNAKTKPREEQYNSTYRV | | VCLISDFYPGAVTVAWK |
| | | VSVLTVLHQDWLNGKEYKCKVSNKALGAPIEKT | | ADSSPVKAGVETTTPSK |
| | | ISKAKGQPREPQVYTLPPSREEMTKNQVSLTCL | | QSNNKYAASSYLSLTPE |
| | | VKGFYPSDIAVEWESNGQPENNYKTTPPVLDSD | | QWKSHRSYSCQVTHEGS |
| | | GSFFLYSKLTVDKSRWQQGNVFSCSVMHEALHN | | TVEKTVAPTECS |
| | | HYTQKSLSLSPGK | | |
| A177- | 5792 | MGWSCIILFLVATATGVHSQLQLQESGPGLMKP | 5980 | MGWSCIILFLVATATGV |
| LALA | | SETLSLTCTVSGGSISSRSYYWGWIRQPPGKGL | | HSQSALTQPASVSGSPG |
| PGhL | | EWIGSVFYSGSTYYNPSLKSRVTISVDTSKNQF | | QSITISCTGTSSDVGGY |
| 2 | | SLKVISVTAADTAVYYCVRQTYDSWTGYSFFYF | | NYVSWYQQHPGKAPKLM |
| | | DYWGQGTLVTVSSASTKGPSVFPLAPSSKSTSG | | IYDVSKRPSGVSNRFSG |
| | | GTAALGCLVKDYFPEPVTVSWNSGALTSGVHTF | | SKSGNTASLTISGLQAE |
| | | PAVLQSSGLYSLSSVVTVPSSSLGTQTYICNVN | | DEADYYCCSYAGSSWVF |
| | | HKPSNTKVDKKVEPKSCDKTHTCPPCPAPEAAG | | GGGTKLTVLGQPKAAPS |
| | | GPSVFLFPPKPKDTLMISRTPEVTCVVVDVSHE | | VTLFPPSSEELQANKAT |
| | | DPEVKFNWYVDGVEVHNAKTKPREEQYNSTYRV | | LVCLISDFYPGAVTVAW |
| | | VSVLTVLHQDWLNGKEYKCKVSNKALGAPIEKT | | KADSSPVKAGVETTTPS |
| | | ISKAKGQPREPQVYTLPPSREEMTKNQVSLTCL | | KQSNNKYAASSYLSLTP |
| | | VKGFYPSDIAVEWESNGQPENNYKTTPPVLDSD | | EQWKSHRSYSCQVTHEG |
| | | GSFFLYSKLTVDKSRWQQGNVFSCSVMHEALHN | | STVEKTVAPTECS |
| | | HYTQKSLSLSPGK | | |
| A178- | 5793 | MGWSCIILFLVATATGVHSEVQLVESGGGLVQP | 5981 | MGWSCIILFLVATATGV |
| LALA | | GGSLKLSCAASGFTFSGSAMHWVRQASGKGLEW | | HSSYELTQPPSVSVSPG |
| PGhL | | VGRIRSKANSYATAYAASVKGRFTISRDDSKNT | | QTASITCSGDKLGDKYA |
| 2 | | AYLQMNSLKTEDTAVYYCTRPMITFGGVIVYDA | | CWYQQKPGQSPVLVIYQ |
| | | FDIWGQGTMVTVSSASTKGPSVFPLAPSSKSTS | | DSKRPSGIPERFSGSNS |
| | | GGTAALGCLVKDYFPEPVTVSWNSGALTSGVHT | | GNTATLTISGTQAMDEA |
| | | FPAVLQSSGLYSLSSVVTVPSSSLGTQTYICNV | | DYYCQAWDSSTVIFGGG |
| | | NHKPSNTKVDKKVEPKSCDKTHTCPPCPAPEAA | | TKLTVLGQPKAAPSVTL |
| | | GGPSVFLFPPKPKDTLMISRTPEVTCVVVDVSH | | FPPSSEELQANKATLVC |
| | | EDPEVKFNWYVDGVEVHNAKTKPREEQYNSTYR | | LISDFYPGAVTVAWKAD |
| | | VVSVLTVLHQDWLNGKEYKCKVSNKALGAPIEK | | SSPVKAGVETTTPSKQS |
| | | TISKAKGQPREPQVYTLPPSREEMTKNQVSLTC | | NNKYAASSYLSLTPEQW |
| | | LVKGFYPSDIAVEWESNGQPENNYKTTPPVLDS | | KSHRSYSCQVTHEGSTV |
| | | DGSFFLYSKLTVDKSRWQQGNVFSCSVMHEALH | | EKTVAPTECS |
| | | NHYTQKSLSLSPGK | | |
| A179- | 5794 | MGWSCIILFLVATATGVHSQVQLQQWGAGLLKP | 5982 | MGWSCIILFLVATATGV |
| LALA | | SETLSLTCAVYGGSFSGYYWSWIRQPPGKGLEW | | HSSYELTQPPSVSVSPG |
| PGhL | | IGEINHSGSTNYNPSLKSRVTISVDTSKNQFSL | | QTARITCSGDALPKKYA |
| 2 | | KLSSVTAADTAVYYCARGWGSSSWYYFDYWGQG | | YWYQQKSGQAPVLVIYE |
| | | TLVTVSSASTKGPSVFPLAPSSKSTSGGTAALG | | DNKRPSGIPERFSGSSS |
| | | CLVKDYFPEPVTVSWNSGALTSGVHTFPAVLQS | | GTMATLTISGAQVEDEA |
| | | SGLYSLSSVVTVPSSSLGTQTYICNVNHKPSNT | | DYYCYSTDSSGNHRVFG |
| | | KVDKKVEPKSCDKTHTCPPCPAPEAAGGPSVEL | | GGTKLTVLGQPKAAPSV |
| | | FPPKPKDTLMISRTPEVTCVVVDVSHEDPEVKF | | TLFPPSSEELQANKATL |
| | | NWYVDGVEVHNAKTKPREEQYNSTYRVVSVLTV | | VCLISDFYPGAVTVAWK |
| | | LHQDWLNGKEYKCKVSNKALGAPIEKTISKAKG | | ADSSPVKAGVETTTPSK |
| | | QPREPQVYTLPPSREEMTKNQVSLTCLVKGFYP | | QSNNKYAASSYLSLTPE |
| | | SDIAVEWESNGQPENNYKTTPPVLDSDGSFFLY | | QWKSHRSYSCQVTHEGS |
| | | SKLTVDKSRWQQGNVFSCSVMHEALHNHYTQKS | | TVEKTVAPTECS |
| | | LSLSPGK | | |
| A180- | 5795 | MGWSCIILFLVATATGVHSQVQLQQWGAGLLKP | 5983 | MGWSCIILFLVATATGV |
| LALA | | SETLSLTCAVYGGSFSGYYWSWIRQPPGKGLEW | | HSSSELTQDPAVSVALG |
| PGhL | | IGEINRSGSTNYNPSLKTRVTISVDTSKNQFSL | | QTVRITCQGDSLRNYYA |
| 2 | | QLSSVTAADTAVYYCARGIFGVGGNWEDPWGQG | | SWYQQKPGQAPVIVIYG |
| | | TLVTVSSASTKGPSVFPLAPSSKSTSGGTAALG | | KNNRPSGIPDRFSGSSS |
| | | CLVKDYFPEPVTVSWNSGALTSGVHTEPAVLQS | | GNTASLTITGAQAEDEA |
| | | SGLYSLSSVVTVPSSSLGTQTYICNVNHKPSNT | | DYYCNSRDSSGNHLVFG |
| | | KVDKKVEPKSCDKTHTCPPCPAPEAAGGPSVFL | | GGTKLTVLGQPKAAPSV |
| | | FPPKPKDTLMISRTPEVTCVVVDVSHEDPEVKF | | TLFPPSSEELQANKATL |
| | | NWYVDGVEVHNAKTKPREEQYNSTYRVVSVLTV | | VCLISDFYPGAVTVAWK |
| | | LHQDWLNGKEYKCKVSNKALGAPIEKTISKAKG | | ADSSPVKAGVETTTPSK |
| | | QPREPQVYTLPPSREEMTKNQVSLTCLVKGFYP | | QSNNKYAASSYLSLTPE |
| | | SDIAVEWESNGQPENNYKTTPPVLDSDGSFFLY | | QWKSHRSYSCQVTHEGS |
| | | SKLTVDKSRWQQGNVFSCSVMHEALHNHYTQKS | | TVEKTVAPTECS |
| | | LSLSPGK | | |
| A181- | 5796 | MGWSCIILFLVATATGVHSQLQLQESGPGLVKP | 5984 | MGWSCIILFLVATATGV |
| LALA | | SETLSLTCTVSGGSISSSSYYWGWIRQPPGKGL | | HSQSALTQPPSASGSPG |
| PGhL | | EWIGSIYYSGSTYYNPSLKSRVTISVDTSKNQF | | QSVTISCTGTSSDVGGY |
| 2 | | SLKLSSVTAADTAVYYCARYSSSWSGFDYWGQG | | NYVSWYQQHPGKAPKLM |
| | | TLVTVSSASTKGPSVFPLAPSSKSTSGGTAALG | | IYEVSKRPSGVPDRFSG |
| | | CLVKDYFPEPVTVSWNSGALTSGVHTFPAVLQS | | SKSGNTASLTVSGLQAE |
| | | SGLYSLSSVVTVPSSSLGTQTYICNVNHKPSNT | | DEADYYCSSYAGSNNFG |
| | | KVDKKVEPKSCDKTHTCPPCPAPEAAGGPSVEL | | GGTKLTVLGQPKAAPSV |
| | | FPPKPKDTLMISRTPEVTCVVVDVSHEDPEVKF | | TLFPPSSEELQANKATL |
| | | NWYVDGVEVHNAKTKPREEQYNSTYRVVSVLTV | | VCLISDFYPGAVTVAWK |
| | | LHQDWLNGKEYKCKVSNKALGAPIEKTISKAKG | | ADSSPVKAGVETTTPSK |
| | | QPREPQVYTLPPSREEMTKNQVSLTCLVKGFYP | | QSNNKYAASSYLSLTPE |
| | | SDIAVEWESNGQPENNYKTTPPVLDSDGSFFLY | | QWKSHRSYSCQVTHEGS |
| | | SKLTVDKSRWQQGNVFSCSVMHEALHNHYTQKS | | TVEKTVAPTECS |
| | | LSLSPGK | | |
| A182- | 5797 | MGWSCIILFLVATATGVHSQLQLQESGPGLVKP | 5985 | MGWSCIILFLVATATGV |
| LALA | | SETLSLTCTVSGGSISSSSYYWGWIRQPPGKGL | | HSSSELTQDPAVSVALG |
| PGhL | | EWIGSIYYSGSTYYNPSLKSRVTISVDTSKNQF | | QTVRITCQGDSLRTYYA |
| 2 | | SLKLSSVTAADTAVYYCARGGSYYVYFDYWGQG | | SWYQQKPGQAPVLVIYG |
| | | TLVTVSSASTKGPSVFPLAPSSKSTSGGTAALG | | KNKRPSGIPDRFSGSSS |
| | | CLVKDYFPEPVTVSWNSGALTSGVHTFPAVLQS | | GNTASLTITGAQAEDEA |
| | | SGLYSLSSVVTVPSSSLGTQTYICNVNHKPSNT | | DYYCNSRDSSGNHWVFG |
| | | KVDKKVEPKSCDKTHTCPPCPAPEAAGGPSVEL | | GGTKLTVLGQPKAAPSV |
| | | FPPKPKDTLMISRTPEVTCVVVDVSHEDPEVKE | | TLFPPSSEELQANKATL |
| | | NWYVDGVEVHNAKTKPREEQYNSTYRVVSVLTV | | VCLISDFYPGAVTVAWK |
| | | LHQDWLNGKEYKCKVSNKALGAPIEKTISKAKG | | ADSSPVKAGVETTTPSK |
| | | QPREPQVYTLPPSREEMTKNQVSLTCLVKGFYP | | QSNNKYAASSYLSLTPE |
| | | SDIAVEWESNGQPENNYKTTPPVLDSDGSFFLY | | QWKSHRSYSCQVTHEGS |
| | | SKLTVDKSRWQQGNVFSCSVMHEALHNHYTQKS | | TVEKTVAPTECS |
| | | LSLSPGK | | |
| A183- | 5798 | MGWSCIILFLVATATGVHSEVQLVESGGGLVQP | 5986 | MGWSCIILFLVATATGV |
| LALA | | GGSLRLSCAASGFTFRSYWMSWVRQAPGKGLEW | | HSSYVLTQPPSVSVAPG |
| PGhL | | VANINQDGSEKYYVDSVKGRFTISRDNAKNSLY | | QTARIICGGDNIGIKNV |
| 2 | | LHMNSLRAEDTAVYYCSRDTDCSSTSCYFNWNP | | HWYQQKPGQAPVLVIYD |
| | | FFDYWGQGTLVTVSSASTKGPSVFPLAPSSKST | | DSDRPSGIPERFSGSNS |
| | | SGGTAALGCLVKDYFPEPVTVSWNSGALTSGVH | | GNTATLTISRVEAGDEA |
| | | TFPAVLQSSGLYSLSSVVTVPSSSLGTQTYICN | | DYCCQVWDSSSDHVVFG |
| | | VNHKPSNTKVDKKVEPKSCDKTHTCPPCPAPEA | | GGTKLTVLGQPKAAPSV |
| | | AGGPSVFLFPPKPKDTLMISRTPEVTCVVVDVS | | TLFPPSSEELQANKATL |
| | | HEDPEVKENWYVDGVEVHNAKTKPREEQYNSTY | | VCLISDFYPGAVTVAWK |
| | | RVVSVLTVLHQDWLNGKEYKCKVSNKALGAPIE | | ADSSPVKAGVETTTPSK |
| | | KTISKAKGQPREPQVYTLPPSREEMTKNQVSLT | | QSNNKYAASSYLSLTPE |
| | | CLVKGFYPSDIAVEWESNGQPENNYKTTPPVLD | | QWKSHRSYSCQVTHEGS |
| | | SDGSFFLYSKLTVDKSRWQQGNVFSCSVMHEAL | | TVEKTVAPTECS |
| | | HNHYTQKSLSLSPGK | | |
| A184- | 5799 | MGWSCIILFLVATATGVHSQLQLQESGPGLVKP | 5987 | MGWSCIILFLVATATGV |
| LALA | | SETLSLTCTVSGGSINSSNFYWGWIRQPPGKGL | | HSSYELTQPPSVSVSPG |
| PGhL | | EWFGSIFYSGFTYYNPSLKSRVTISVDTSKNQF | | QTASITCSGDKLGDKYT |
| 2 | | SLKLTSVTAADTAVYYCARGGYSYGLNWFDPWG | | CWYQQKPGQSPVLVIYQ |
| | | QGTLVTVSSASTKGPSVFPLAPSSKSTSGGTAA | | DIKRPSGIPDRFSGSNS |
| | | LGCLVKDYFPEPVTVSWNSGALTSGVHTFPAVL | | GNTATLTISGTQAMDEA |
| | | QSSGLYSLSSVVTVPSSSLGTQTYICNVNHKPS | | DYYCQAWDSSTVVFGGG |
| | | NTKVDKKVEPKSCDKTHTCPPCPAPEAAGGPSV | | TKLTVLGQPKAAPSVTL |
| | | FLFPPKPKDTLMISRTPEVTCVVVDVSHEDPEV | | FPPSSEELQANKATLVC |
| | | KFNWYVDGVEVHNAKTKPREEQYNSTYRVVSVL | | LISDFYPGAVTVAWKAD |
| | | TVLHQDWLNGKEYKCKVSNKALGAPIEKTISKA | | SSPVKAGVETTTPSKQS |
| | | KGQPREPQVYTLPPSREEMTKNQVSLTCLVKGF | | NNKYAASSYLSLTPEQW |
| | | YPSDIAVEWESNGQPENNYKTTPPVLDSDGSFF | | KSHRSYSCQVTHEGSTV |
| | | LYSKLTVDKSRWQQGNVFSCSVMHEALHNHYTQ | | EKTVAPTECS |
| | | KSLSLSPGK | | |
| A185- | 5800 | MGWSCIILFLVATATGVHSQLQLQESGPGLVKP | 5988 | MGWSCIILFLVATATGV |
| LALA | | SETLSLTCTVSGGSISSSSYYWGWIRQPPGKGL | | HSSSELTQDPAVSVALG |
| PGhL | | EWIGSIYYSGSTYYNPSLKSRVTISVDTSKNQF | | QTVRITCQGDSLRSYYA |
| 2 | | SLKLSSVTAADTAVYYCARTYYDFWSGYLNWED | | SWYQQKPGQAPVLVIYG |
| | | PWGQGTLVTVSSASTKGPSVFPLAPSSKSTSGG | | KNNRPSGIPDRFSGSSS |
| | | TAALGCLVKDYFPEPVTVSWNSGALTSGVHTFP | | GNTASLTITGAQAEDEA |
| | | AVLQSSGLYSLSSVVTVPSSSLGTQTYICNVNH | | DYYCNSRDSSGNHVVFG |
| | | KPSNTKVDKKVEPKSCDKTHTCPPCPAPEAAGG | | GGTKLTVLGQPKAAPSV |
| | | PSVFLFPPKPKDTLMISRTPEVTCVVVDVSHED | | TLFPPSSEELQANKATL |
| | | PEVKFNWYVDGVEVHNAKTKPREEQYNSTYRVV | | VCLISDFYPGAVTVAWK |
| | | SVLTVLHQDWLNGKEYKCKVSNKALGAPIEKTI | | ADSSPVKAGVETTTPSK |
| | | SKAKGQPREPQVYTLPPSREEMTKNQVSLTCLV | | QSNNKYAASSYLSLTPE |
| | | KGFYPSDIAVEWESNGQPENNYKTTPPVLDSDG | | QWKSHRSYSCQVTHEGS |
| | | SFFLYSKLTVDKSRWQQGNVFSCSVMHEALHNH | | TVEKTVAPTECS |
| | | YTQKSLSLSPGK | | |
| A186- | 5801 | MGWSCIILFLVATATGVHSQVQLQQWGAGLLKP | 5989 | MGWSCIILFLVATATGV |
| LALA | | SETLSLTCAVYGGSFSGYYWSWIRQPPGKGLEW | | HSQSALTQPPSASGSPG |
| PGhL | | IGEINRGGSTNYNPSLKSRVTISVDTSKNQFSL | | QSVTISCTGTSSDVGGY |
| 2 | | KLSSVTAADTAVYYCARWRNYYDSSGSPYWYED | | NYVSWYQQHPGKAPKLM |
| | | LWGRGTLVTVSSASTKGPSVFPLAPSSKSTSGG | | IYEVSKRPSGVPDRFSG |
| | | TAALGCLVKDYFPEPVTVSWNSGALTSGVHTFP | | SKSGNTASLTVSGLQAE |
| | | AVLQSSGLYSLSSVVTVPSSSLGTQTYICNVNH | | DEADYYCSSYAGSNNWV |
| | | KPSNTKVDKKVEPKSCDKTHTCPPCPAPEAAGG | | FGGGTKLTVLGQPKAAP |
| | | PSVFLFPPKPKDTLMISRTPEVTCVVVDVSHED | | SVTLFPPSSEELQANKA |
| | | PEVKFNWYVDGVEVHNAKTKPREEQYNSTYRVV | | TLVCLISDFYPGAVTVA |
| | | SVLTVLHQDWLNGKEYKCKVSNKALGAPIEKTI | | WKADSSPVKAGVETTTP |
| | | SKAKGQPREPQVYTLPPSREEMTKNQVSLTCLV | | SKQSNNKYAASSYLSLT |
| | | KGFYPSDIAVEWESNGQPENNYKTTPPVLDSDG | | PEQWKSHRSYSCQVTHE |
| | | SFFLYSKLTVDKSRWQQGNVFSCSVMHEALHNH | | GSTVEKTVAPTECS |
| | | YTQKSLSLSPGK | | |
| A187- | 5802 | MGWSCIILFLVATATGVHSQLQLQESGPGLVKP | 5990 | MGWSCIILFLVATATGV |
| LALA | | SETLSLTCTVSGGSISSSGYYWGWIRQSPGKGL | | HSQSALTQPASVSGSPG |
| PGhL | | EWIGSFYYSGSTYYNPSLKSRVTISVDTSKNQF | | QSITISCTGTSSDVGGY |
| 2 | | SLKLSSVTAADTAVYYCARQGRITMVRGVIPFD | | NYVSWYQQHPGKAPKLM |
| | | YWGQGTLVTVSSASTKGPSVFPLAPSSKSTSGG | | IYEVSNRPSGVSNRFSG |
| | | TAALGCLVKDYFPEPVTVSWNSGALTSGVHTFP | | SKSGNTASLTISGLQAE |
| | | AVLQSSGLYSLSSVVTVPSSSLGTQTYICNVNH | | DEADYYCSSYTSSSTLV |
| | | KPSNTKVDKKVEPKSCDKTHTCPPCPAPEAAGG | | FGGGTKLTVLGQPKAAP |
| | | PSVFLFPPKPKDTLMISRTPEVTCVVVDVSHED | | SVTLFPPSSEELQANKA |
| | | PEVKFNWYVDGVEVHNAKTKPREEQYNSTYRVV | | TLVCLISDFYPGAVTVA |
| | | SVLTVLHQDWLNGKEYKCKVSNKALGAPIEKTI | | WKADSSPVKAGVETTTP |
| | | SKAKGQPREPQVYTLPPSREEMTKNQVSLTCLV | | SKQSNNKYAASSYLSLT |
| | | KGFYPSDIAVEWESNGQPENNYKTTPPVLDSDG | | PEQWKSHRSYSCQVTHE |
| | | SFFLYSKLTVDKSRWQQGNVFSCSVMHEALHNH | | GSTVEKTVAPTECS |
| | | YTQKSLSLSPGK | | |
| A188- | 5803 | MGWSCIILFLVATATGVHSQVQLQQWGAGLLKP | 5991 | MGWSCIILFLVATATGV |
| LALA | | SETLSLTCAVYGGSFSGYYWSWIRQPPGKGLEW | | HSQTVVTQEPSLTVSPG |
| PGhL | | IGEINHSGSTNYNPSLKSRVTISVDTSKNQFSL | | GTVTLTCGSSTGAVTSG |
| 2 | | KLSSVTAADTAVYYCAGGYCSSTSCRYNWNYGG | | HYPYWFQQKPGQAPRTL |
| | | WFDPWGQGTLVTVSSASTKGPSVFPLAPSSKST | | IYDTSNKHSWTPARFSG |
| | | SGGTAALGCLVKDYFPEPVTVSWNSGALTSGVH | | SLLGGKAALTLSGAQPE |
| | | TFPAVLQSSGLYSLSSVVTVPSSSLGTQTYICN | | DEAEYYCLLSYSGARVE |
| | | VNHKPSNTKVDKKVEPKSCDKTHTCPPCPAPEA | | GGGTKLTVLGQPKAAPS |
| | | AGGPSVFLFPPKPKDTLMISRTPEVTCVVVDVS | | VTLFPPSSEELQANKAT |
| | | HEDPEVKFNWYVDGVEVHNAKTKPREEQYNSTY | | LVCLISDFYPGAVTVAW |
| | | RVVSVLTVLHQDWLNGKEYKCKVSNKALGAPIE | | KADSSPVKAGVETTTPS |
| | | KTISKAKGQPREPQVYTLPPSREEMTKNQVSLT | | KQSNNKYAASSYLSLTP |
| | | CLVKGFYPSDIAVEWESNGQPENNYKTTPPVLD | | EQWKSHRSYSCQVTHEG |
| | | SDGSFFLYSKLTVDKSRWQQGNVFSCSVMHEAL | | STVEKTVAPTECS |
| | | HNHYTQKSLSLSPGK | | |

It is understood that, while particular embodiments have been illustrated and described, various modifications may be made thereto and are contemplated herein. It is also understood that the disclosure is not limited by the specific examples provided herein. The description and illustration of embodiments and examples of the disclosure herein are not intended to be construed in a limiting sense. It is further understood that all aspects of the disclosure are not limited to the specific depictions, configurations or relative proportions set forth herein, which may depend upon a variety of conditions and variables. Various modifications and variations in form and detail of the embodiments and examples of the disclosure will be apparent to a person skilled in the art. It is therefore contemplated that the disclosure also covers any and all such modifications, variations and equivalents.

1-46. (canceled)

47. A method for treating or inhibiting cancer metastasis in a subject, the method comprising:
administering a pharmaceutically effective amount of a composition that:
i) inhibits or reduces binding of an erythropoietin (EPO) protein to an EPO receptor (EPOR) complex, wherein said EPOR complex comprises (a) at least two EPOR subunits or (b) at least one EPOR subunit and at least one CD131 subunit;
ii) inhibits or reduces formation of an EPO receptor (EPOR) complex, wherein said EPOR complex comprises (a) at least two EPOR subunits or (b) at least one EPOR subunit and at least one CD131 subunit;
iii) inhibits or reduces activation of an EPO receptor (EPOR) complex on a plurality of macrophages, wherein said EPOR complex comprises (a) at least two EPOR subunits or (b) at least one EPOR subunit and at least one CD131 subunit;
iv) reduces an amount of circulating EPO protein in said subject; or
v) reduces an expression level of an EPO receptor (EPOR) complex on a plurality of macrophages, wherein said EPOR complex comprises (a) at least two EPOR subunits or (b) at least one EPOR subunit and at least one CD131 subunit;
thereby:
a) treating the cancer metastasis;
b) inhibiting formation of a pre-metastatic niche in a distant site that is different from a location of a primary tumor; or
c) inhibiting a metastatic nodule growth.

48. The method of claim 47, wherein said EPOR complex is a homo-EPOR complex that comprises at least two EPOR subunits.

49. The method of claim 48, wherein said homo-EPOR complex comprises or consists of two EPOR subunits.

50. The method of claim 47, wherein said EPOR complex is a hetero-EPOR complex that comprises at least one EPOR subunit and at least one CD131 subunit.

51. The method of claim 50, wherein said hetero-EPOR complex comprises or consists of an EPOR subunit and a CD131 subunit.

52. The method of claim 47, wherein said subject has an existing metastatic cancer.

53. The method of claim 47, wherein said subject does not have an existing metastatic cancer.

54. The method of claim 47, further comprising inhibiting or reducing exacerbating said primary tumor.

55. The method of claim 47, further comprising increasing CD8+ T cell/regulatory T cell ratio.

56. The method of claim 47, further comprising inhibiting development of regulatory T cells (Tregs).

57. The method of claim 55, wherein CD8+ T cells expresses Cluster of Differentiation 45 (CD45), CD3, CD8, Perforin, Interferon gamma (IFNγ), Granzyme B, or tumor necrosis factor alpha (TNFα).

58. The method of claim 56, wherein Tregs express Cluster of Differentiation 4 (CD4), CD25, CD127, Forkhead Box P3 (FoxP3), CD39, protein tyrosine phosphatase receptor type C (CD45RA), Interleukin-2 (IL-2), or a Cytotoxic T-Lymphocyte Associated Protein 4 (CTLA-4).

59. The method of claim 47, further comprising rendering cancer cells in said cancer metastasis sensitive to an immune checkpoint inhibitor.

60. The method of claim 59, wherein said immune checkpoint inhibitor comprises a Cytotoxic T-Lymphocyte Associated Protein 4 (CTLA-4) inhibitor, a Programmed Death 1 (PD-1) inhibitor, or a Programmed Death Ligand 1 (PD-L1) inhibitor.

61. The method of claim 47, wherein said distant site is different from a location of said primary tumor or said distant site is a tissue that is distinct from a tissue where said primary tumor is located.

62. The method of claim 47, wherein said composition comprises an antibody or a functional fragment thereof.

63. The method of claim 62, wherein said antibody or said functional fragment thereof selectively binds to an EPOR subunit or said EPOR complex.

64. The method of claim 47, wherein said composition comprises a compound or a salt thereof, wherein said compound is an inhibitor of hypoxia-inducible factor (HIF), IL-1α, IL-1β, TNF-α, IL-6, estrogen receptors, phospholipase C-γ1, or Cb1/p85/Episin-1 pathway.

65. The method of claim 47, wherein said composition comprises a plurality of lipid nanoparticles (LNPs) comprising siRNA directed to hetero-EPOR on macrophages.

66. The method of claim 47, wherein said composition comprises an engineered EPOR comprising an extracellular domain (ECD) of said EPOR subunit.

67. The method of claim 62, wherein said composition further comprises a second antibody or a functional fragment thereof that selectively binds to an immune checkpoint protein.

68. The method of claim 67, wherein said immune checkpoint protein comprises PD-1, PD-L1, or CTLA-4.

69. The method of claim 68, wherein said immune checkpoint protein is PD-1.

70. A method for treating or inhibiting cancer metastasis in a subject, the method comprising:
administering a pharmaceutically effective amount of a composition that comprises (i) an antibody or a functional fragment thereof, or (ii) an engineered fusion protein or a functional fragment thereof,
wherein said antibody or said functional fragment thereof selectively binds to a first target and a second target,
wherein the first target comprises an erythropoietin (EPO) protein, an EPOR subunit, an EPOR complex, or a combination thereof,
wherein the second target comprises an immune checkpoint protein, and
wherein said antibody or said functional fragment thereof comprises a first antigen-binding domain and a second antigen-binding domain.
